(19) United States
(12) Patent Application Publication
(10) Pub. No.: US 2024/0342292 A1
(43) Pub. Date: Oct. 17, 2024

(54) Compounds for the Degradation of EGFR Kinase

(71) Applicant: BeiGene Switzerland GmbH, Basel (CH)

(72) Inventors: Bailin Lei, Beijing (CN); Huaqing Liu, Beijing (CN); Songzhe Han, Beijing (CN); Yizhou Zhao, Beijing (CN); Zhiwei Wang, Beijing (CN); Guanjun Liu, Beijing (CN); Haijun Liu, Beijing (CN)

(73) Assignee: BeiGene Switzerland GmbH, Basel (CH)

(21) Appl. No.: 18/676,648

(22) Filed: May 29, 2024

Related U.S. Application Data
(63) Continuation of application No. PCT/CN2022/135011, filed on Nov. 29, 2022.

(30) Foreign Application Priority Data
Nov. 30, 2021 (WO) PCT/CN2021/134437
Mar. 31, 2022 (WO) PCT/CN2022/084630
Sep. 8, 2022 (WO) PCT/CN2022/117922
Oct. 25, 2022 (WO) PCT/CN2022/127441

(51) Int. Cl. A61K 47/54

(52) U.S. Cl. CPC A61K 47/545

(57) ABSTRACT

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad Sci. 2001, 98: 8554-9; Sakamoto K. M. et al., Methods Enzymol. 2005; 399:833-847.). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF 114) (Spradlin, J. N. et al. Nat. Chem. Biol. 2019, 15, 747-755) and DDB1 And CUL4 Associated Factor 16 (DCAF16)(Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Sientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347.; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018: 61: 6685-6704. Winter G E. et al., Science 2015:348:1376-1381.) In the recent years, PROTACs as useful modulators promote the selective degradation of a wide range of target proteins have been reported in antitumor studies. (Lu J. et al., (Chem Biol. 2015; 22(6):755-763; Ottis P. et al., Chem Biol. 2017: 12(4):892-898.; Crews C. M. et al., J Med Chem. 2018: 61(2):403-404: Neklesa T. K. et al., Pharmacol Ther 2017, 174:138-144.; Cermakova K. et al., Molecules. 2018, 23(8).; An S. et al., EBioMedicine, 2018.; Lebraud H. et al., Essays Biochem. 2017; 61(5): 517-527.: Sun Y H. et al., Cell Res. 2018; 28:779-81; Toure M. et al., Angew Chem Int Ed Engl. 2016; 55(6):1966-1973: Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, WO2018071606, WO2021178920, WO2021127283, WO2021127190, WO202111871 and WO202111913.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137.). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707.). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611.). The first-generation EGFR tyrosine kinase inhibitors (EGFR-TKIs) gefitinib and erlotinib have approved for NSCLC patients with EGFR activation mutations (M. Maemondo, N. Engl. J. Med 362 (2010) 2380-2388.). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582.). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562.). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562, US20190106417, WO202157882, WO2021123087, WO2021133809. WO2021168074, WO2021208918 and WO2021216440.

Although, a number of EGFR-targeting PROTACs which were designed to degrade EGFR mutant proteins have been published (Zhang X., et al. Eur. J. Med. Chem. 2020, 192, 112199.: Zhang H. et al. Eur. J. Med Chem. 2020, 189, 112061.; Lu X, Med. Res. Rev. 2018, 38(5):1550-1581. He K., et al. Bioorg. Med Chem. Lett. 2020, 15, 127167.). Most of the published molecules are based on first, second, and third generation of EGFR inhibitors (WO2021023233, WO2019121562 and WO2018119441) or allosteric EGFR inhibitors (WO2021127561). However, there were no data which showed those EGFR-Targeting PROTACs degrading all the main EGFR mutations, Such as Del19, L858R, Del19/T790M, L858R/T790M, Dell9/T790M, C797S. L858R-T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

The compounds described herein or salts thereof are useful in the treatment of a disease that can be affected by EGFR modulation. The present invention provides the use of the compounds described herein or a pharmaceutically acceptable salt thereof in the manufacture of a medicament for the treatment of a disease that can be affected by EGFR modulation. The present invention further provides a compound described herein or a pharmaceutically acceptable salt thereof, for use in the treatment of a disease that can be affected by EGFR modulation. The present application further provides a method of treating a proliferative disorder, comprising administering to a subject in need thereof a therapeutically effective amount of the compounds described herein or a pharmaceutically acceptable salt thereof.

Aspect 1. A compound of Formula (I):

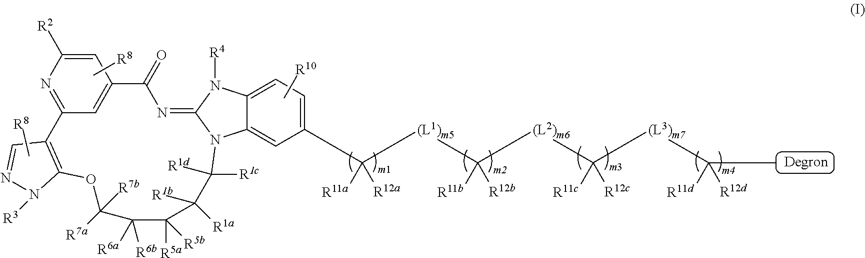

or a N-oxide thereof, or a pharmaceutically acceptable salt thereof, or a stereoisomer thereof, or a deuterated analog thereof, or a prodrug thereof.
wherein:
R³ and R⁴ are each independently absence, hydrogen, —C₁₋₈alkyl, —C₂₋₈alkenyl, —C₂₋₈alkynyl or —C₃₋₈cycloalkyl: each said —C₁₋₈alkyl, —C₂₋₈alkenyl, —C₂₋₈alkynyl or —C₃₋₈cycloalkyl is optionally substituted with at least one substituent selected from hydrogen, halogen, —C₁₋₈alkoxy, —C₃₋₈cycloalkyl or —CN;
R^{1a}, R^{1b}, R^{1c}, R^{1d}, R^{5a}, R^{3a}, R^{3b}, R^{6b}, R^{7a}, R^{7b}, R², R⁸, R⁹ and R¹⁰ are each independently absence, hydrogen, halogen, —C₁₋₈alkyl, —C₂₋₈-alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or

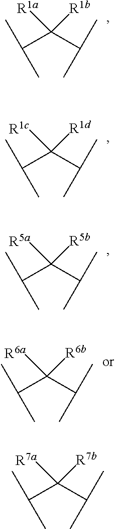

can be optionally replaced with O or NR^{a};
two geminal or adjacent substituent from R^{1a}, R^{1b}, R^{1c}, R^{1d}, R^{5a}, R^{5b}, R^{6a}, R^{6b}, R^{7a} and R^{7b} with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
   R^{11a}, R^{11b}, R^{11c}, R^{11d}, R^{12a}, R^{12b}, R^{12c} and R^{12d} are each independently absence, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN; or
R^{11a} and R^{11b} together with the carbon atoms to which they are attached, form a 3- to 12-membered ring, said ring comprising 0-3 heteroatoms independently selected from nitrogen, oxygen or sulfur: said ring is optionally substituted with at least one substituent selected from hydrogen, halogen, —C₁₋₈alkyl, —C₂₋₈alkenyl, —C₂₋₈alkynyl, —C₂₋₈alkoxy or —CN;
R^{11a} and R^{12a} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
R^{11c} and R^{12c} together with the carbon atoms to which they are attached, form a 3- to 12-membered ring, said ring comprising 0-3 heteroatoms independently selected from nitrogen, oxygen or sulfur; said ring is optionally substituted with at least one substituent selected from hydrogen, halogen, —C₁₋₈alkyl, —C₂₋₈alkenyl, —C₂₋₈alkynyl, —C₁₋₈alkoxy or —CN:
R^{11d} and R^{12d} together with the carbon atoms to which they are attached, form a 3- to 12-membered ring, said ring comprising 0-3 heteroatoms independently selected from nitrogen, oxygen or sulfur, said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
L¹ is independently selected from —O—, —NR—, —C(O)—, *^{L1}—C(O)NR—**^{L1}, *^{L1}—C(O)O—**^{L1},

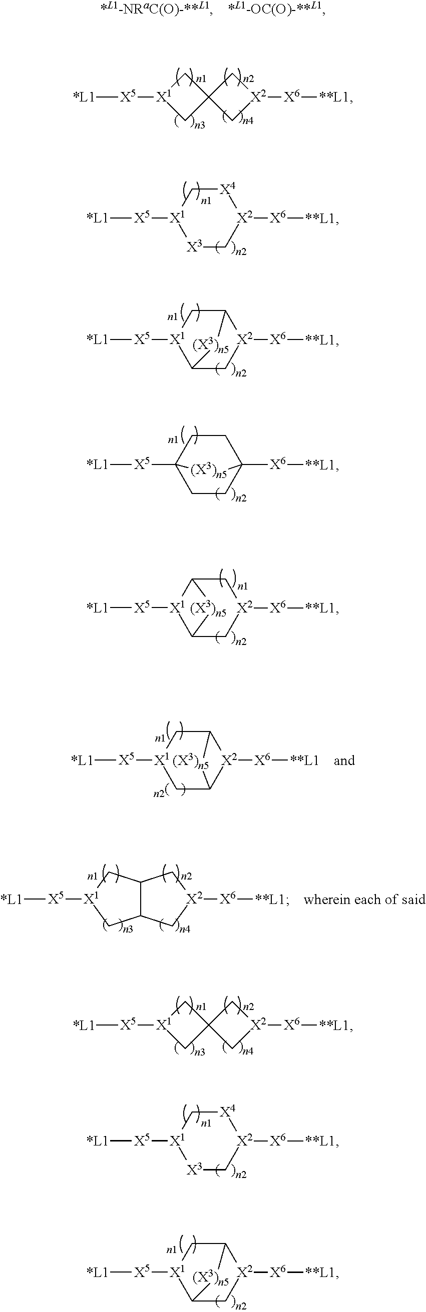

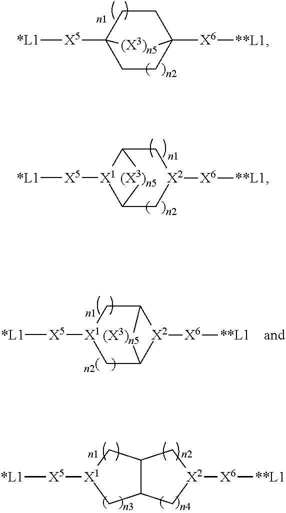

is optionally substituted with at least one R^{L1c};
wherein *^{L1} refers to the position attached to the

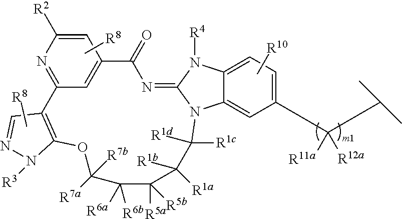

moiety, and **^{L1} refers to the position attached to the

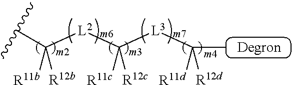

moiety;

L² is independently selected from —O—, —NR^{a}—, —C(O), *^{L2}—C(O)NR^{a}—**^{L2}, *^{L2}—C(O)O—**^{L2},

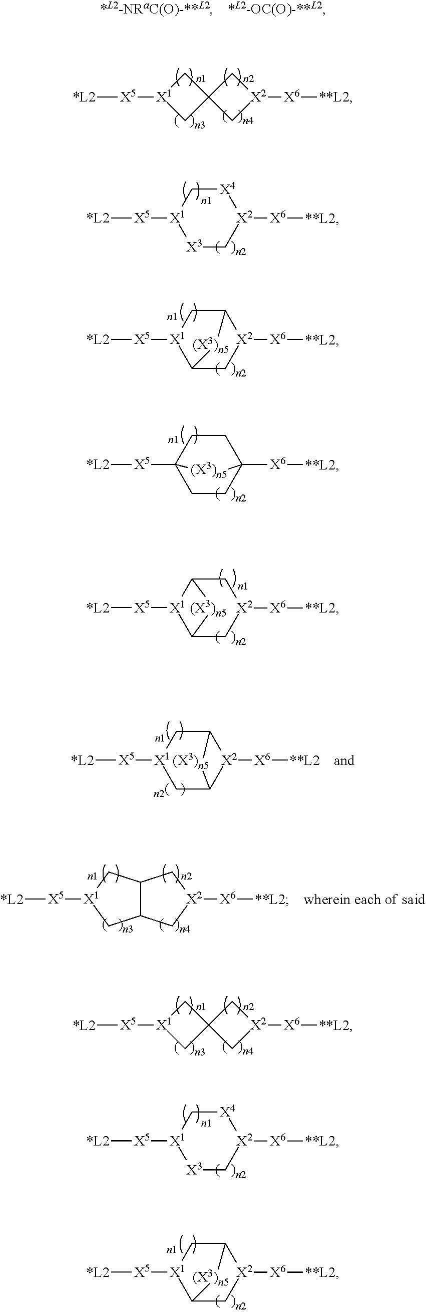

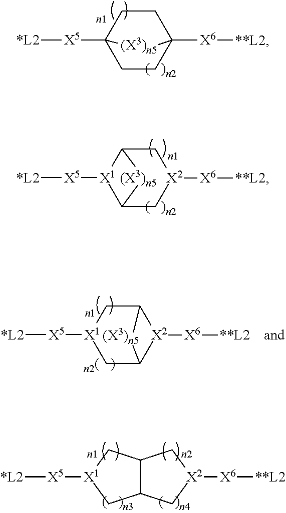

is optionally substituted with at least one R^{L2c};
wherein *^{L2} refers to the position attached to the

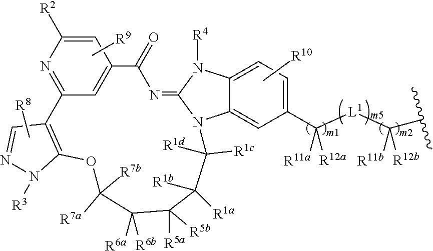

moiety, and **^{L2} refers to the position attached to the

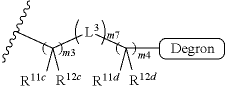

moiety;
L³ is independently selected from —O—, —NR^{a}—, —C(O)—, *^{L3}—C(O)NR^{a}—**^{L3}, *^{L3}—C(O)O—**^{L3}, *^{L3}—NR^{a}C(O)—**^{L3}, *^{L3}—OC(O)—**^{L3},

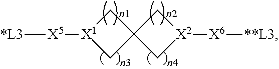

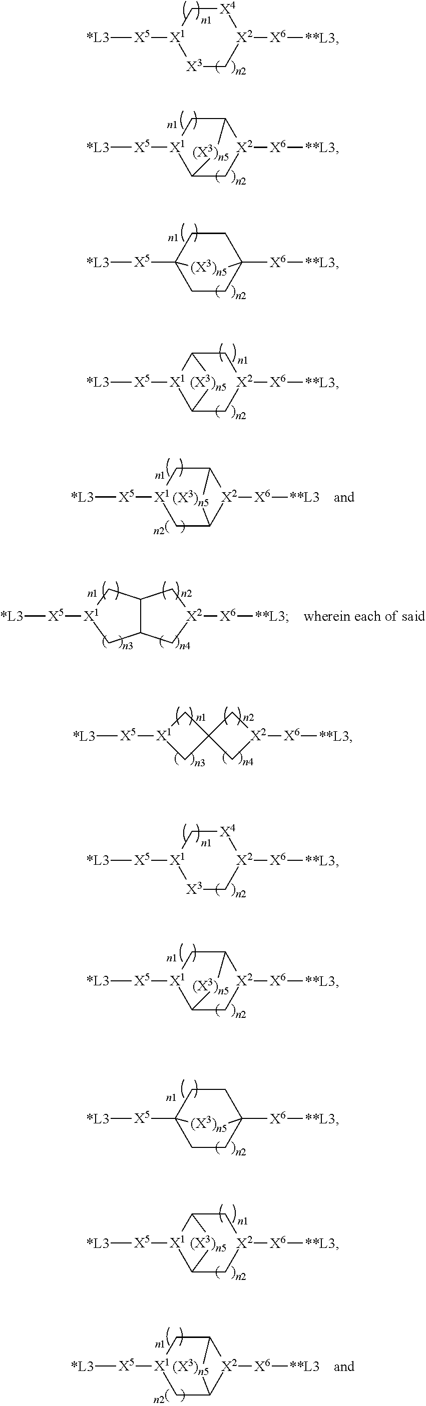

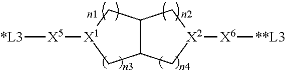

is optionally substituted with at least one R^{L3c};
wherein *^{L3} refers to the position attached to the

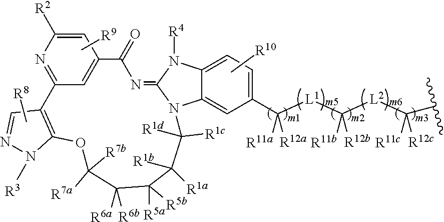

moiety, and **^{L3} refers to the position attached to the

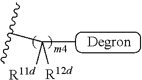

moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

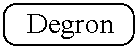

is selected from

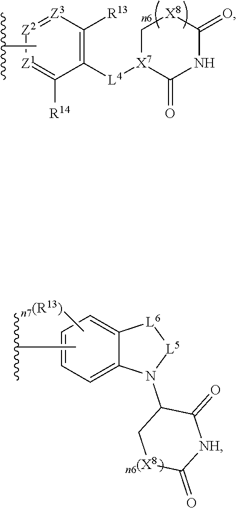

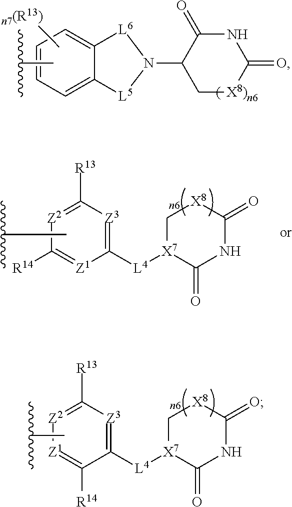

Z¹, Z² and Z³ are each independently N or CR^{z}, provided that Z¹, Z² and Z³ are not N at the same time;
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

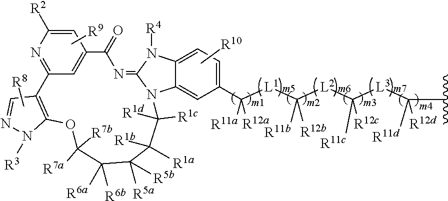

moiety is linked to the

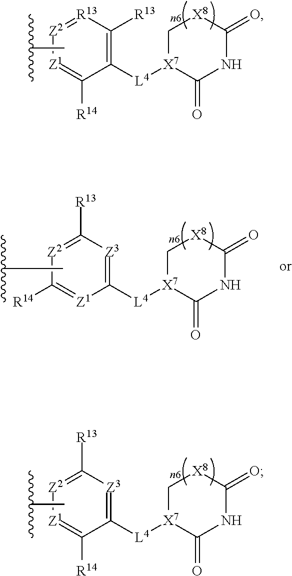

moiety via any one of Z¹, Z² or Z³ which is CR^{z} and R^{z} is absence;

R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl:
R¹³ and R¹⁴ are each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, CONR^{13a}R^{13b}, —NR^{13a}R^{13b}, —NR^{13a}COR^{13b}, —NR^{13a}CO₂R^{13b}, or —NR^{13a}SO₂R^{13b}: each of —C₁₋₈alkyl, —C₂₋₈alkenyl, —C₂₋₈alkynyl, —C₂₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl. —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d}.
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl:
L⁴, L⁵ and L⁶ are each independently selected from a absence, single bond, —O—, —NR*—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR, or N;
at each occurrence, X³, X⁴ and X⁵ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur, said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m1, m2, m3 and m4 are each independently 0, 1 or 2; provided that m1+m2+m3+m4≤4;
m5, m6 and m7 are each independently 0 or 1; provided that m5+m6+m7≥1;
n1, n2, n3, n4 and n5 are each independently 0, 1, 2 or 3:
n6, n7 and n8 are each independently 0, 1, 2, 3 or 4;
provided that:
for any one of L¹, L² or L³, when X¹ is N, X⁵ is single bond, absence, —C(O)—; and/or when X² is N, X⁶ is single bond, absence, —C(O)—;
when L¹ is

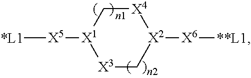

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L² is

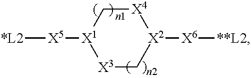

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is

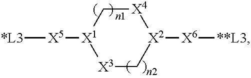

when X¹ is N, X⁵ is single bond, absence, —C(O)—; and/or X³ is —CR^{b}—; when X² is N, X⁶ is single bond, absence, —C(O)—, and/or X⁴ is —CR^{a}R^{b}.

Aspect 2. The compound of aspect 1, wherein the compound is selected from formula (II)

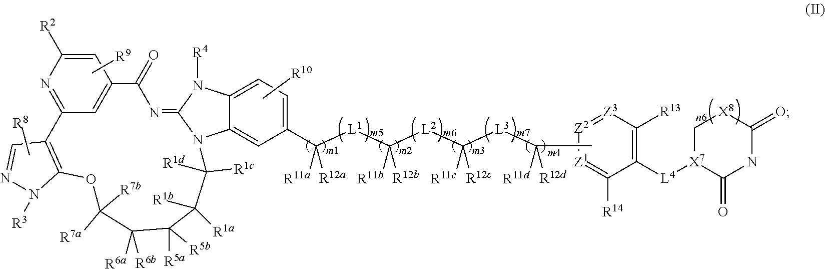

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, L⁴, Z¹, Z², Z³, X⁷, X⁸, m1, m2, m3, m4, m5, m6, m7 and n6 are defined as Aspect 1.

Aspect 3. The compound of any one of the preceding aspects, wherein the compound is selected from formula (111)

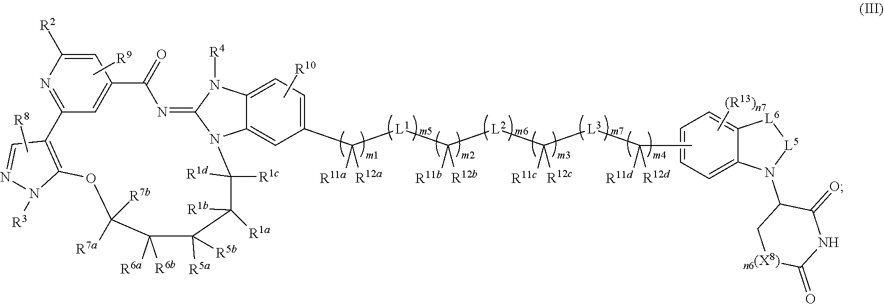

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, L¹, L², L³, L⁴, L⁵, L⁶, X⁸, m1, m2, m3, m4, m5, m6, m7, n6 and n7 are as defined in any one of the preceding aspects.

Aspect 4. The compound of any one of the preceding aspects, wherein the compound is selected from formula (IV)

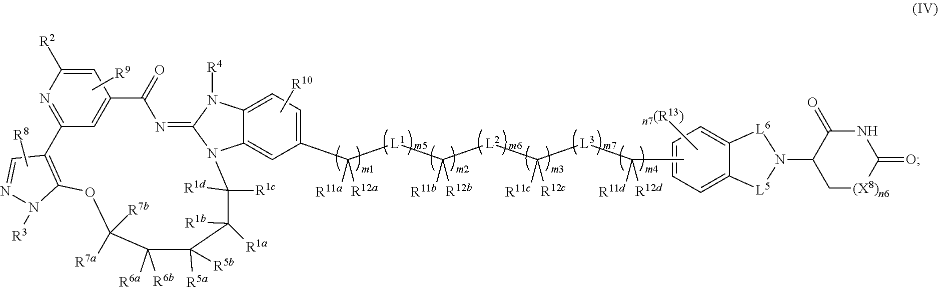

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, L¹, L², L³, L⁵, L⁶, X, m1, m2, m3, m4, m5, m6, m7, n6 and n7 are as defined in any one of the preceding aspects.

Aspect 5. The compound of any one of the preceding aspects, wherein the compound is selected from formula (Va), (Vb), (Vc) or (Vd),

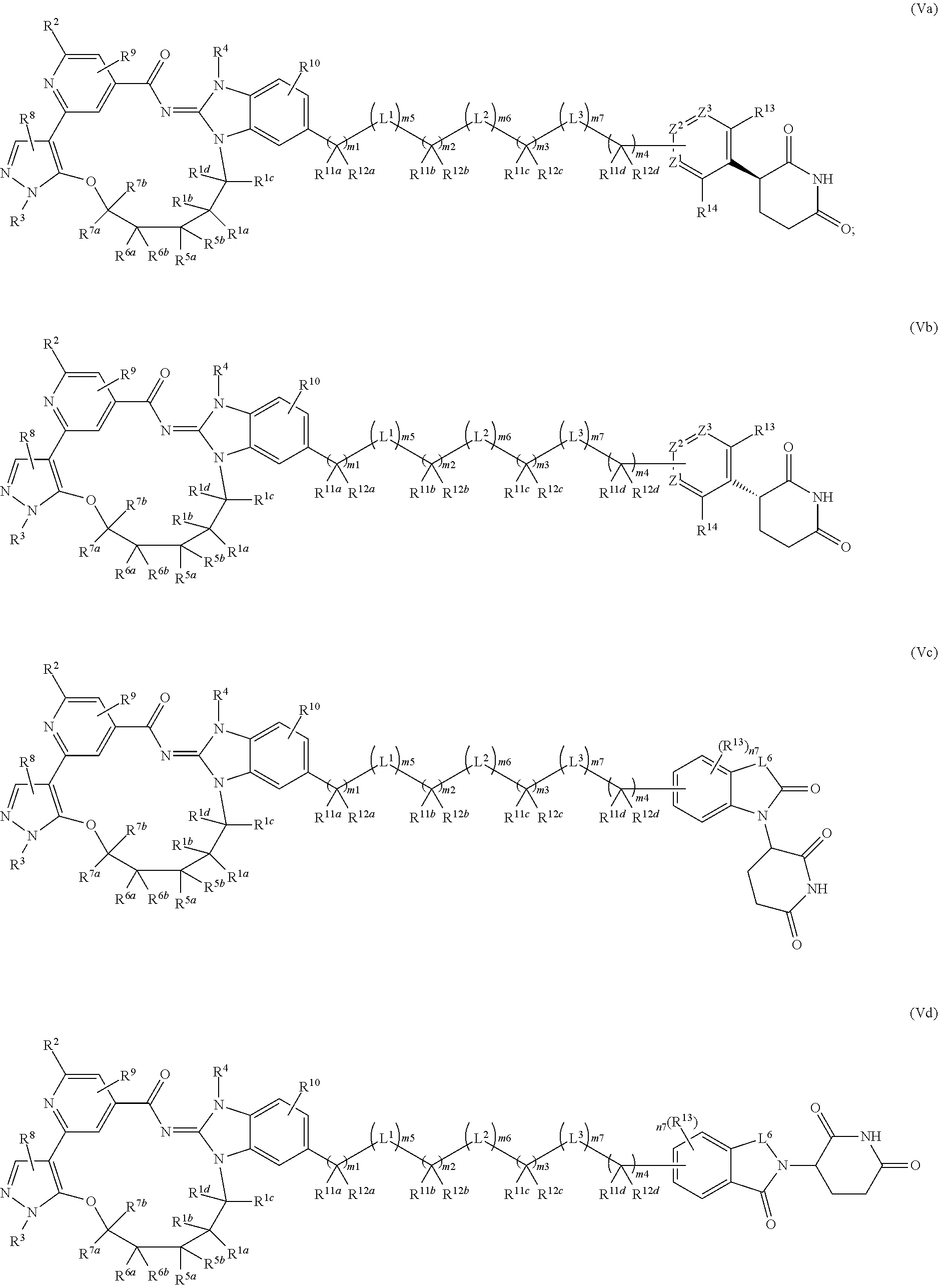

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, L⁶, Z¹, Z², Z³, m1, m2, m3, m4, m5, m6, m7 and n7 are as defined in any one of the preceding aspects.

Aspect 6. The compound of any one of the preceding aspects, wherein the compound is selected from formula (VIa), (VIb), (VIc), (Vid) or (VIe),

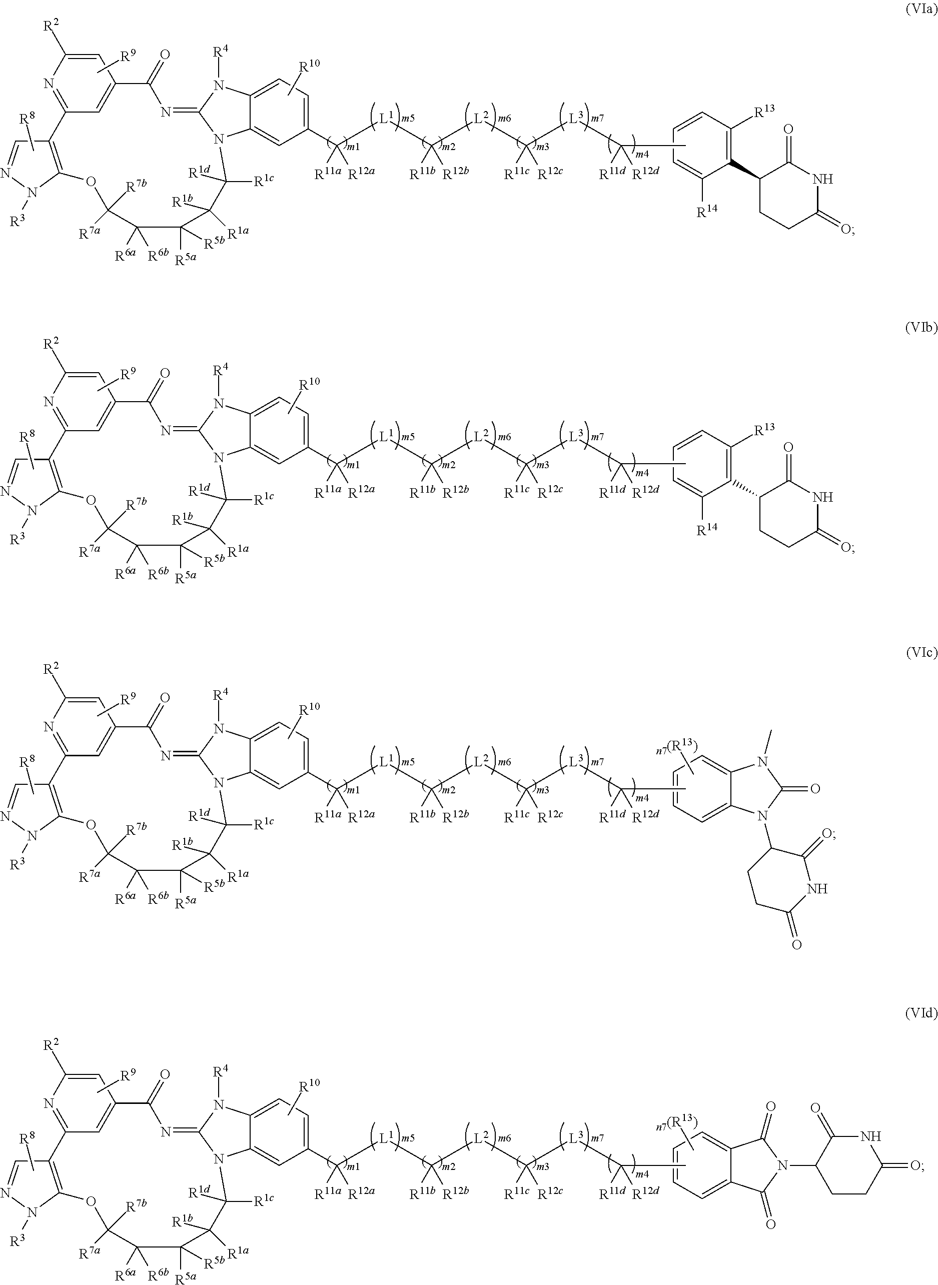

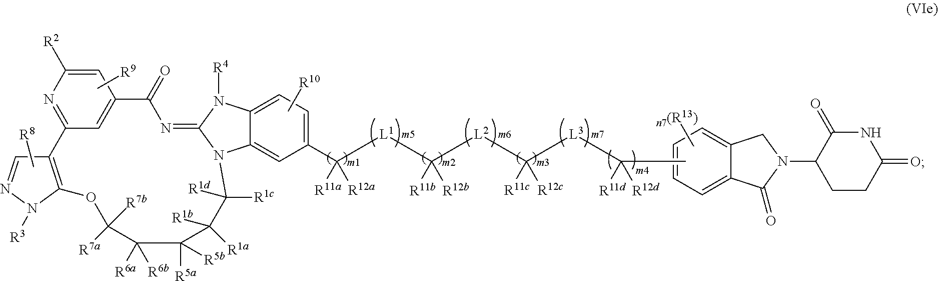

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, m1, m2, m3, m4, m5, m6, m7 and n7 are as defined in any one of the preceding aspects.

Aspect 7. The compound of any one of the preceding aspects, wherein the compound is selected from formula (VIIa), (VIIb), (VIIc), (VIId) or (VIIe).

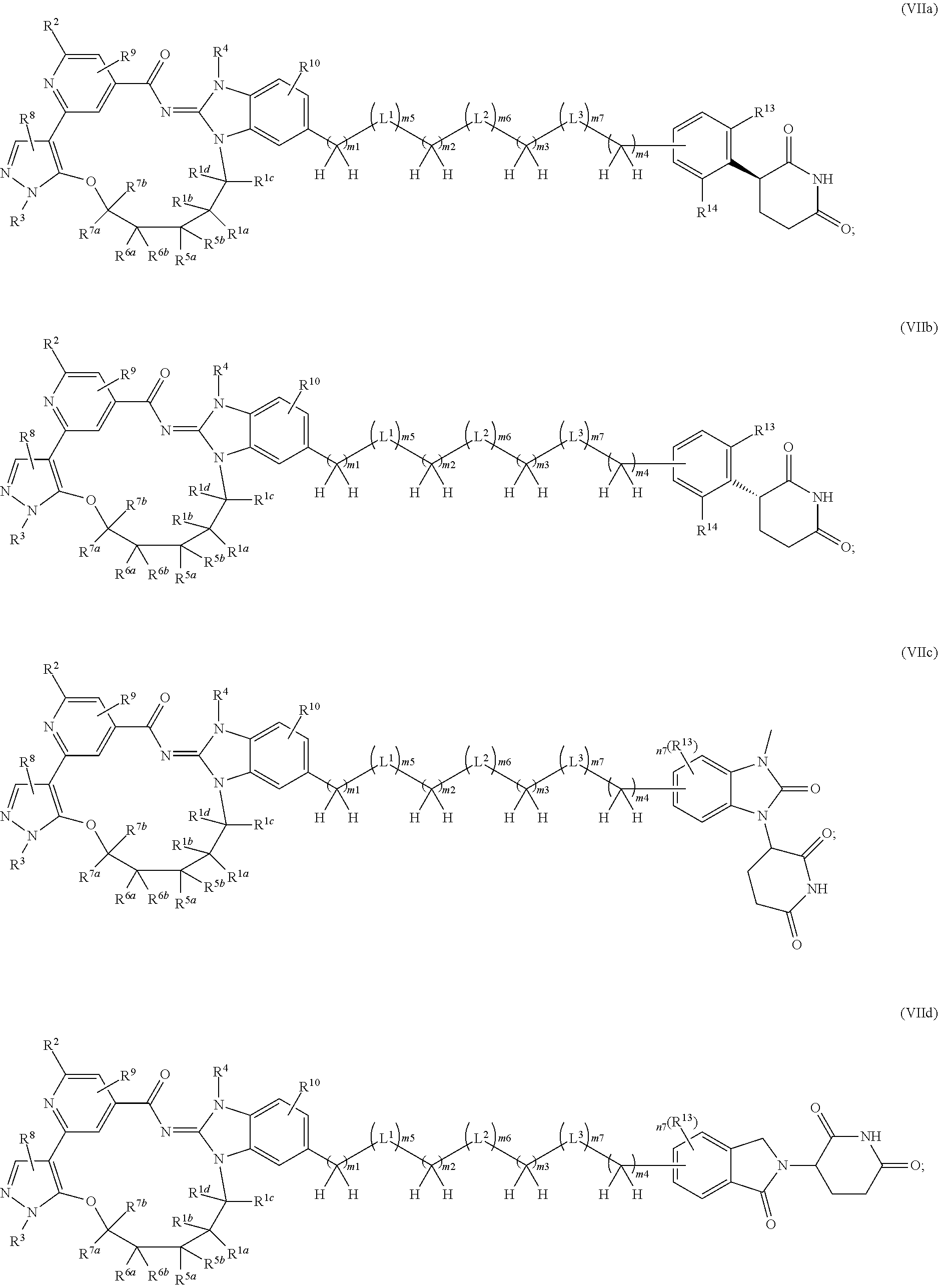

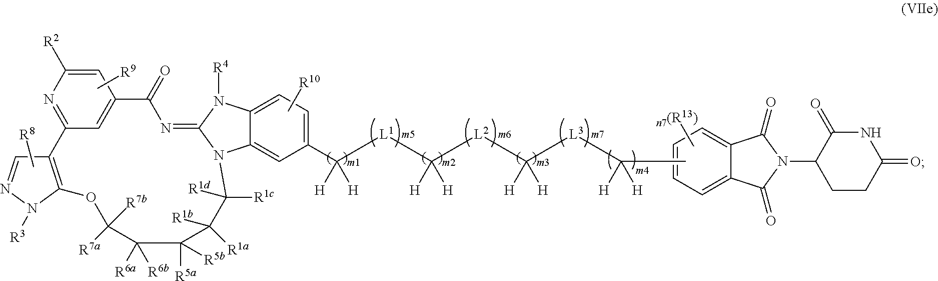

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R¹³, R¹⁴, L¹, L², L³, m1, m2, m3, m4, m5, m6, m7 and n7 are as defined in any one of the preceding aspects.

Aspect 8. The compound of any one of the preceding aspects, wherein the compound is selected from formula (VIIIa), (VIIIb), (VIIIc) or (VIIId),

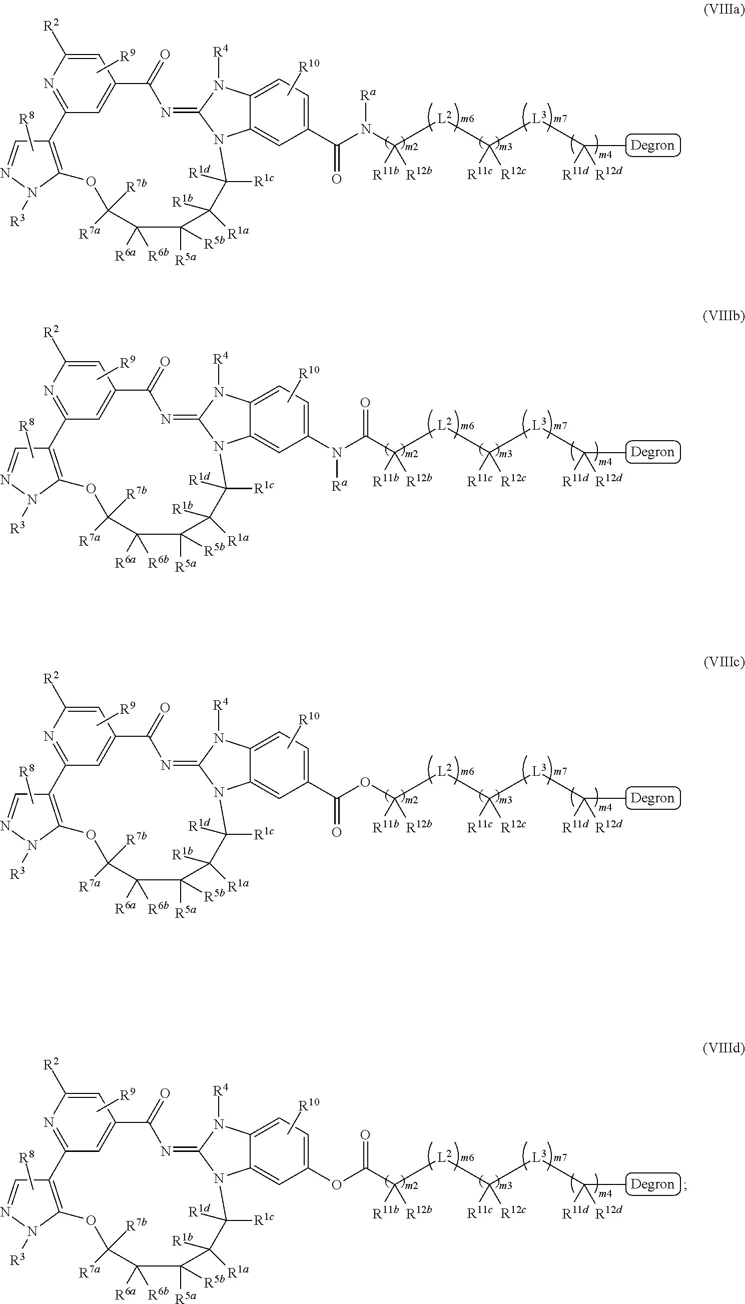

wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11b}, R^{11c}, R^{11d}, R^{12b}, R^{12c}, R^{12d}, R¹³, R¹⁴, L², L³, m2, m3, m4, m6, m7 and Degron are as defined in any one of the preceding aspects.

Aspect 9. The compound of any one of the preceding aspects, wherein m1+m2+m3+m4≤3.

Aspect 10. The compound of any one of the preceding aspects, wherein m1+m2+m3+m4=1, 2 or 3, preferably, m1+m2+m3+m4=1 or 2.

Aspect 11. The compound of any one of the preceding aspects, wherein the number of —CH₂— in

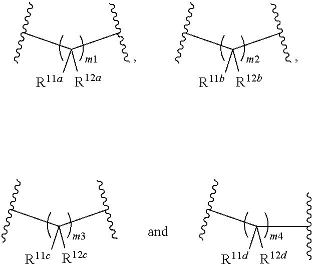

moieties is no more than 4, preferably is no more than 3, even more preferably is no more than 2.

Aspect 12. The compound of any one of the preceding aspects, wherein R¹ and R⁴ are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl or cyclooctyl; said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl or cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN;

Aspect 13. The compound of any one of the preceding aspects, wherein R³ and R⁴ are each independently hydrogen, methyl, ethyl, propyl, butyl, pentyl, hexyl, heptyl, octyl, cyclopropyl, cyclobutyl, cyclopentyl, cyclohexyl, cycloheptyl or cyclooctyl; preferably R³ is independently methyl or cyclopropyl, and R⁴ is hydrogen.

Aspect 14. The compound of any one of the preceding aspects, wherein R^{1a}, R^{1b}, R^{1c}, R^{13a}, R^{5a}, R^{5b}, R^{6a}, R^{6b}, R^{7a}, R^{7b}, R², R⁸, R⁹ and R¹⁰ are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN; wherein each said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl and cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

Aspect 15. The compound of any one of the preceding aspects, wherein R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₃; preferably R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently hydrogen, F, Cl, methyl, methoxy, cyclopropyl, —CF₃ or —CHF₂, —CH₂OCH₃; more preferably, R² is hydrogen, methyl, methoxy, cyclopropyl or —CF₃; R⁸ is hydrogen, F, methyl, —CF₃ or —CHF₂; R⁹ is hydrogen or F; R¹⁰ is hydrogen, F, Cl, methyl or —CH₂OCH₃; R^{1a}, R^{1b}, R^{1c}, R^{1d}, R^{5a}, R^{5b}, R^{6a}, R^{6b}, R^{7a} and R^{7b} are each independently hydrogen, F or methyl.

Aspect 16. The compound of any one of the preceding aspects, wherein (R^{1a} and R^{1b}), (R^{1c} and R^{1d}), (R^{5a} and R^{5b}), (R^{6a} and R^{6b}), (R^{7a} and R^{7b}), (R^{1a} and R^{1b}), (R^{1a} and R^{1d}), (R^{1b} and R^{1c}), (R^{1b} and R^{1d}), (R^{1a} and R^{5a}), (R^{1a} and R^{5b}), (R^{1b} and R^{5a}), (R^{1b} and R^{5b}), (R^{5a} and R^{6a}), (R^{5a} and R^{6b}), (R^{5b} and R^{6a}), (R^{5b} and R^{6b}), (R^{6a} and R^{7a}), (R^{6a} and R^{7b}), (R^{6b} and R^{7a}) or (R^{6b} and R^{7b}) with the carbon atoms to which they are attached, form a 3-, 4-, 5-, 6-, 7- or 8-membered ring, said ring comprising 0-3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl.

Aspect 17. The compound of any one of the preceding aspects, wherein the

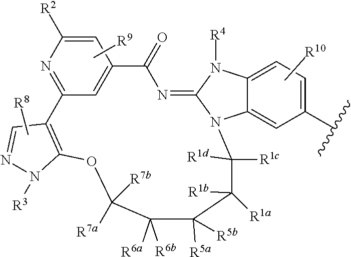

moiety is selected from

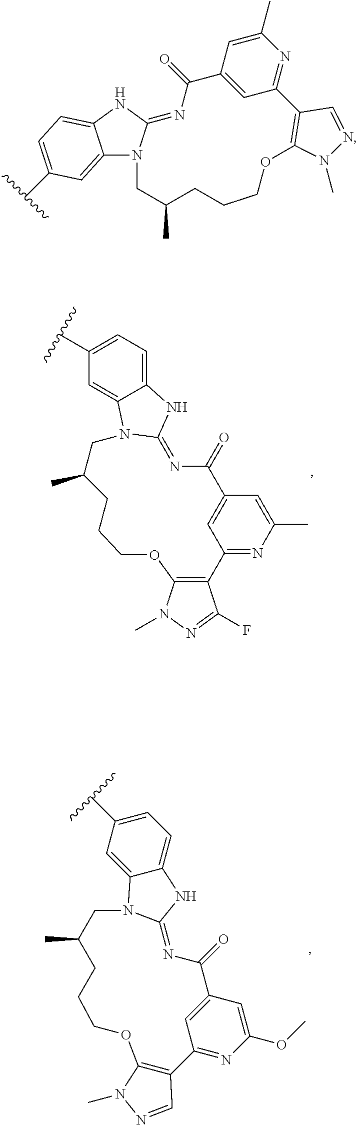

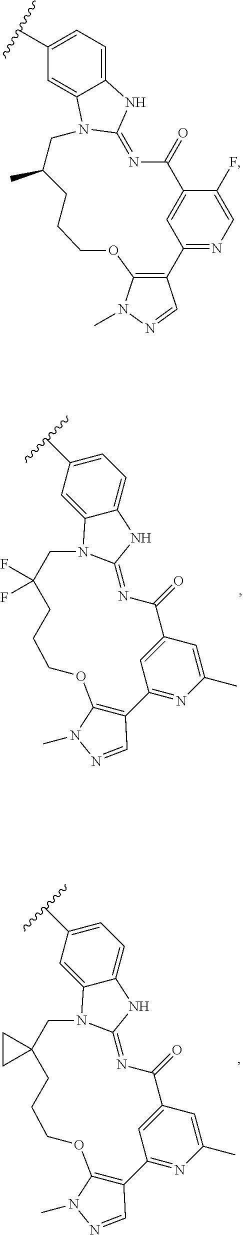 [SMILES: *c1ccc2c(c1)N1C[C@H](C)CCCOc3c(cnn3C)-c3cc(c(F)cn3)C(=O)/N=C/1N2 ; *c1ccc2c(c1)N1CC(F)(F)CCCOc3c(cnn3C)-c3cc(cc(C)n3)C(=O)/N=C/1N2 ; *c1ccc2c(c1)N1CC3(CCCOc4c(cnn4C)-c4cc(cc(C)n4)C(=O)/N=C/1N2)CC3]

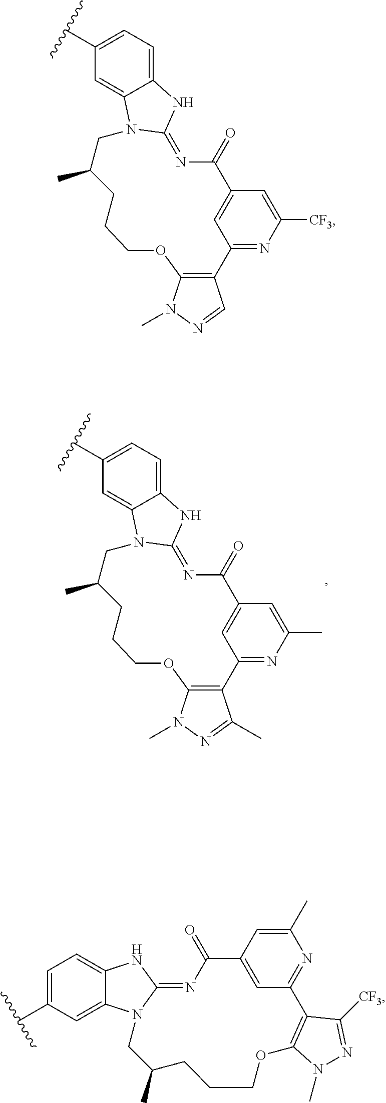

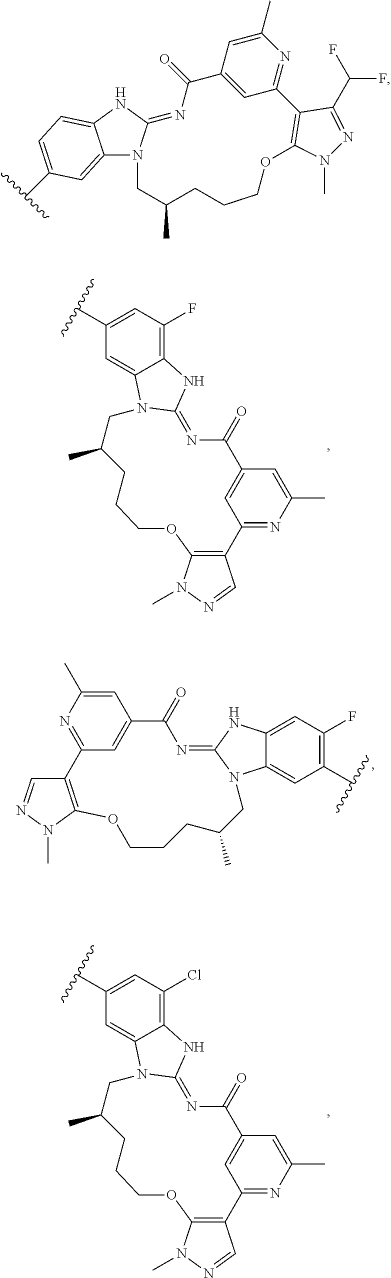

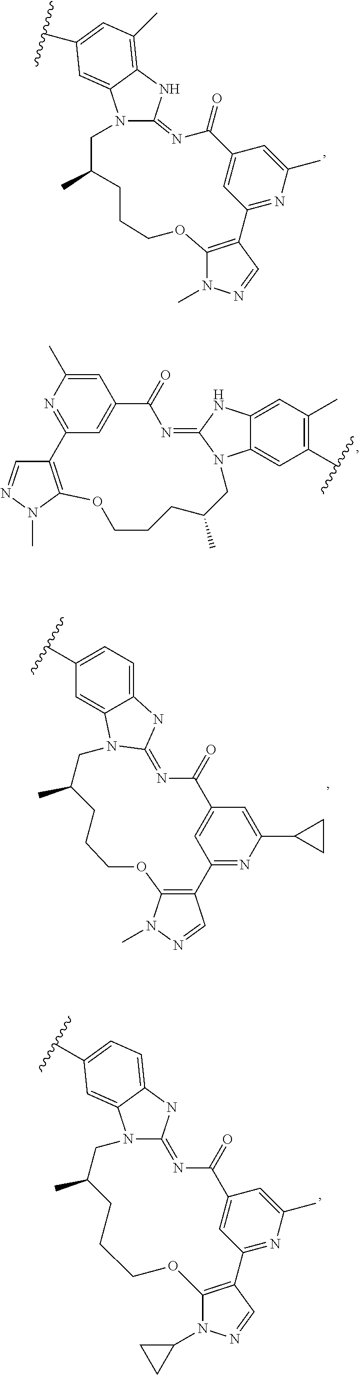

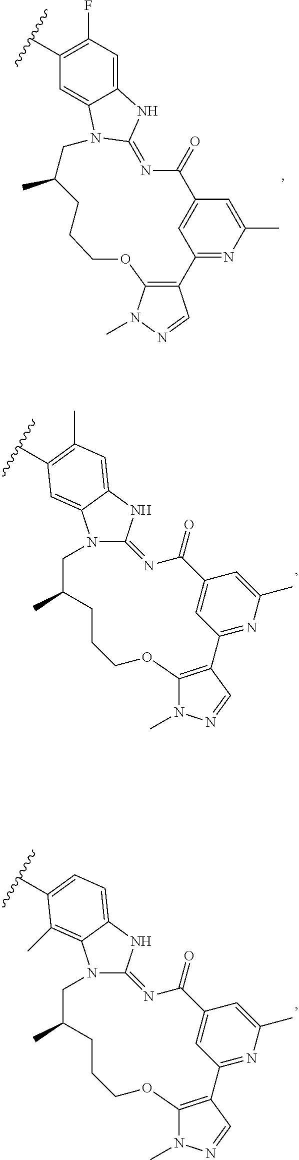

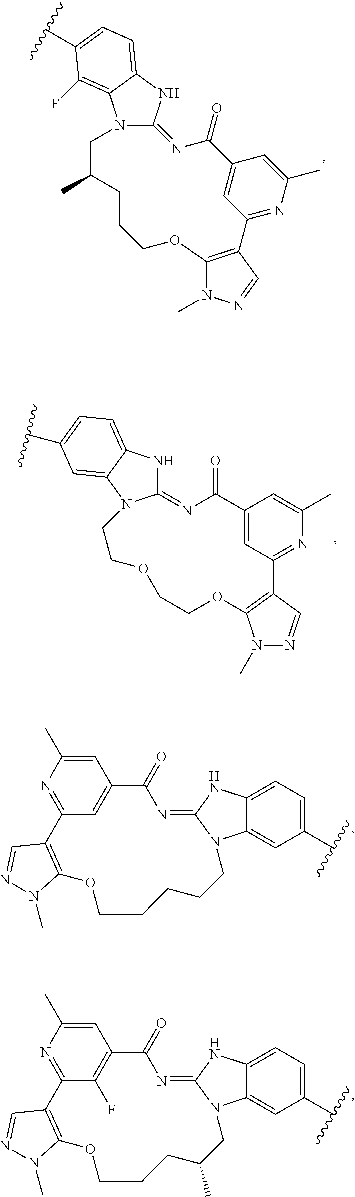 [SMILES: *c1ccc2c(c1F)N1C[C@H](C)CCCOc3c(cnn3C)-c3cc(cc(C)n3)C(=O)/N=C/1N2 ; *c1ccc2c(c1)N1CCOCCOc3c(cnn3C)-c3cc(cc(C)n3)C(=O)/N=C/1N2 ; *c1ccc2c(c1)N1CCCCCOc3c(cnn3C)-c3cc(cc(C)n3)C(=O)/N=C/1N2 ; *c1ccc2c(c1)N1C[C@H](C)CCCOc3c(cnn3C)-c3nc(C)cc(c3F)C(=O)/N=C/1N2]

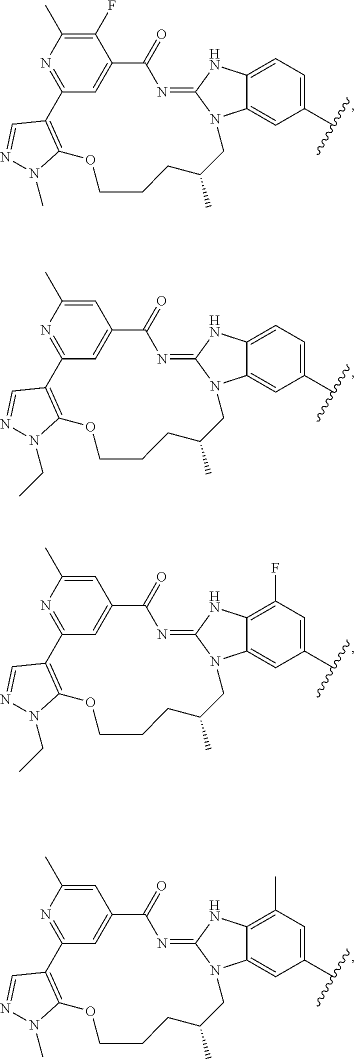 [SMILES: *c1ccc2c(c1)N1C[C@H](C)CCCOc3c(cnn3C)-c3cc(c(F)c(C)n3)C(=O)/N=C/1N2 ; *c1ccc2c(c1)N1C[C@H](C)CCCOc3c(cnn3CC)-c3cc(cc(C)n3)C(=O)/N=C/1N2 ; *c1cc(F)c2c(c1)N1C[C@H](C)CCCOc3c(cnn3CC)-c3cc(cc(C)n3)C(=O)/N=C/1N2 ; *c1cc(C)c2c(c1)N1C[C@H](C)CCCOc3c(cnn3C)-c3cc(cc(C)n3)C(=O)/N=C/1N2]

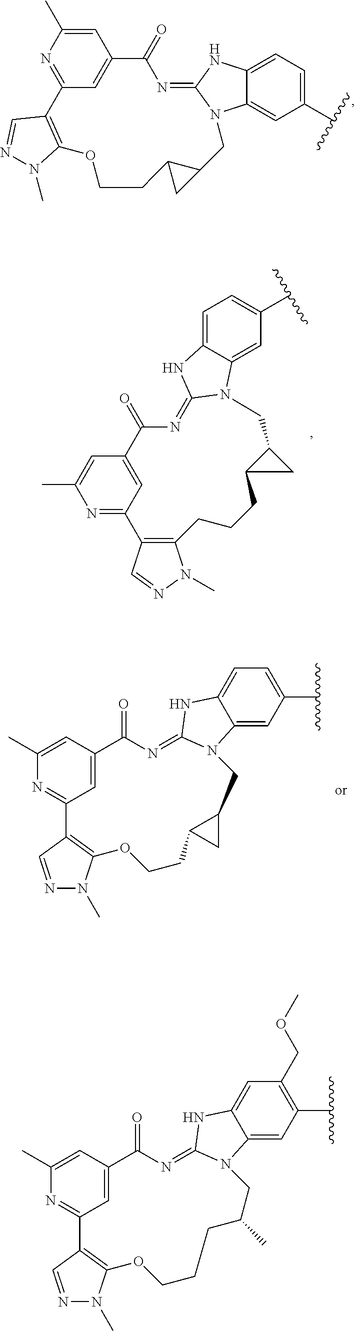 [SMILES: *c1ccc2c(c1)N1CC3CC3CCOc3c(cnn3C)-c3cc(cc(C)n3)C(=O)/N=C/1N2 ; *c1ccc2c(c1)N1C[C@@H]3C[C@H]3CCCc3c(cnn3C)-c3cc(cc(C)n3)C(=O)/N=C/1N2 ; *c1ccc2c(c1)N1C[C@H]3C[C@@H]3CCOc3c(cnn3C)-c3cc(cc(C)n3)C(=O)/N=C/1N2 ; *c1cc2c(cc1COC)N/C1=N\C(=O)c3cc(C)nc(c3)-c3cnn(C)c3OCCC[C@@H](C)CN12]

Aspect 18. The compound of any one of the preceding aspects, wherein R^{11a}, R^{11b}, R^{11c}, R^{11d}, R^{12a}, R^{12b}, R^{12c} and R^{12d} are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl: each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl is optionally substituted with at least one substituent selected from hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN; or

(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur; said ring is optionally substituted with at least one substituent selected from hydrogen, halogen, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN.

Aspect 19. The compound of any one of the preceding aspects, wherein R^{11a}, R^{11b}, R^{11c}, R^{11d}, R^{12a}, R^{12b}, R^{12c} and R^{12d} are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl; preferably, R^{11a}, R^{11b}, R^{11c}, R^{11d}, R^{12a}, R^{12b}, R^{12c} and R^{12d} are each independently hydrogen, F, Cl, Br, I, methyl, ethyl or propyl: more preferably, R^{11a}, R^{11b}, R^{11c}, R^{11d}, R^{12a}, R^{12b}, R^{12c} and R^{12d} are each independently hydrogen or methyl; or
(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur: preferably (R^{11a} and R^{12a}), (R^{11b} and R^{12b}), (R^{11c} and R^{12c}) or (R^{11d} and R^{12d}) together with the carbon atoms to which they are attached, form a 3-, 4- or 5-membered cycloalkyl ring.

Aspect 20. The compound of any one of the preceding aspects, wherein L¹ is selected from —O—, —C(O)—, —N(R^{a})—, *^{L1}—C(O)N(R^{a})—**^{L1}, *^{L1}—C(O)O—**^{L1}, *^{L1}—N(R^{a})C(O)—**^{L1}, *^{L1}—OC(O)—**^{L1},

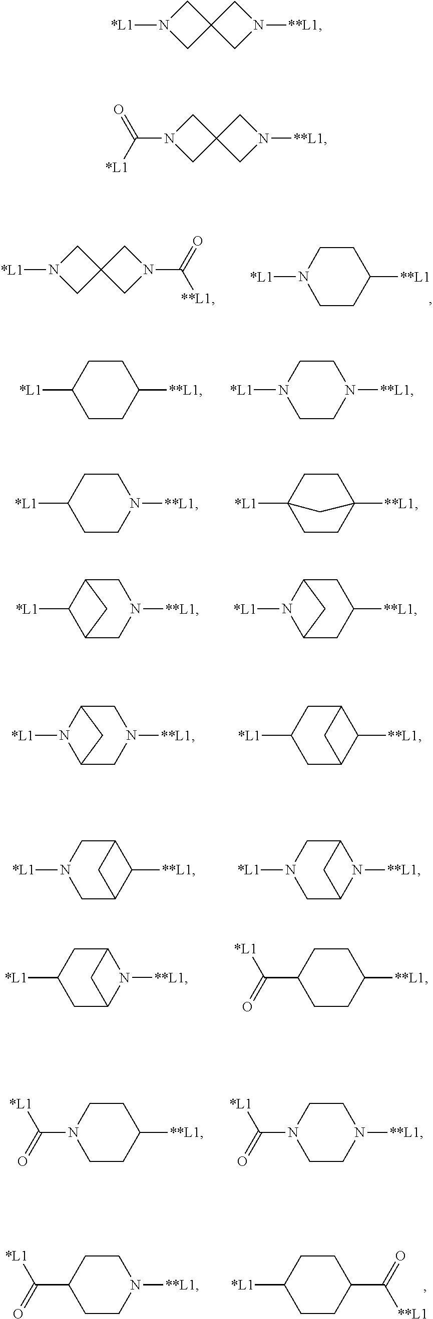

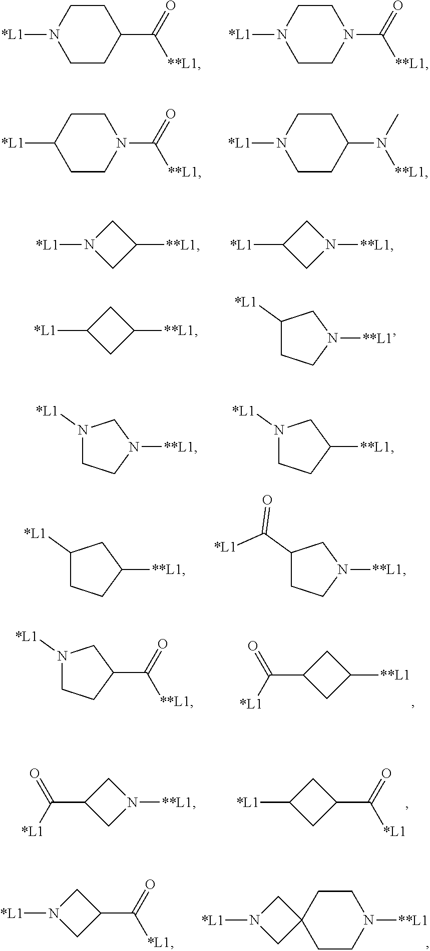

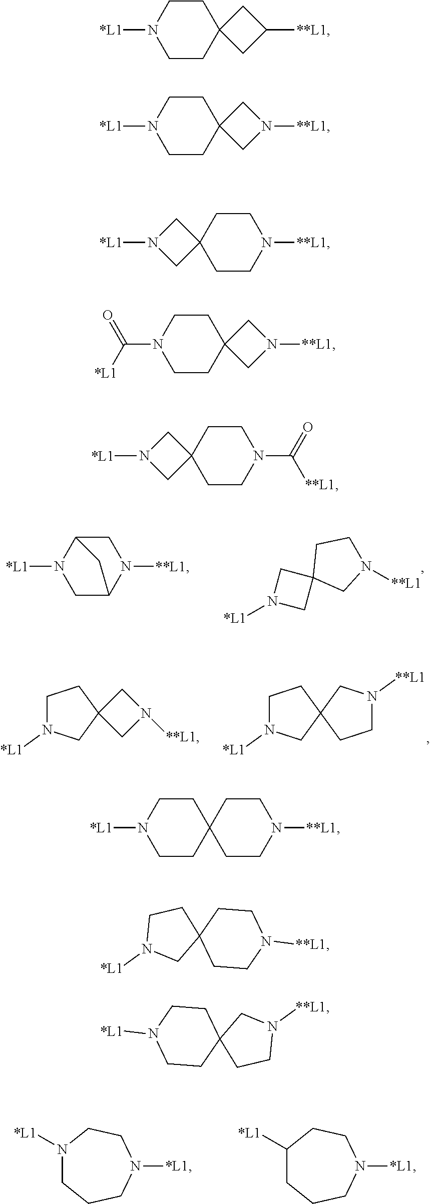

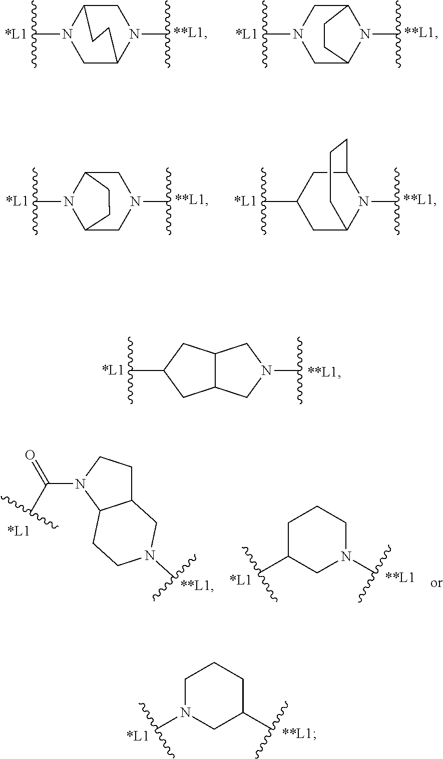

wherein each of said

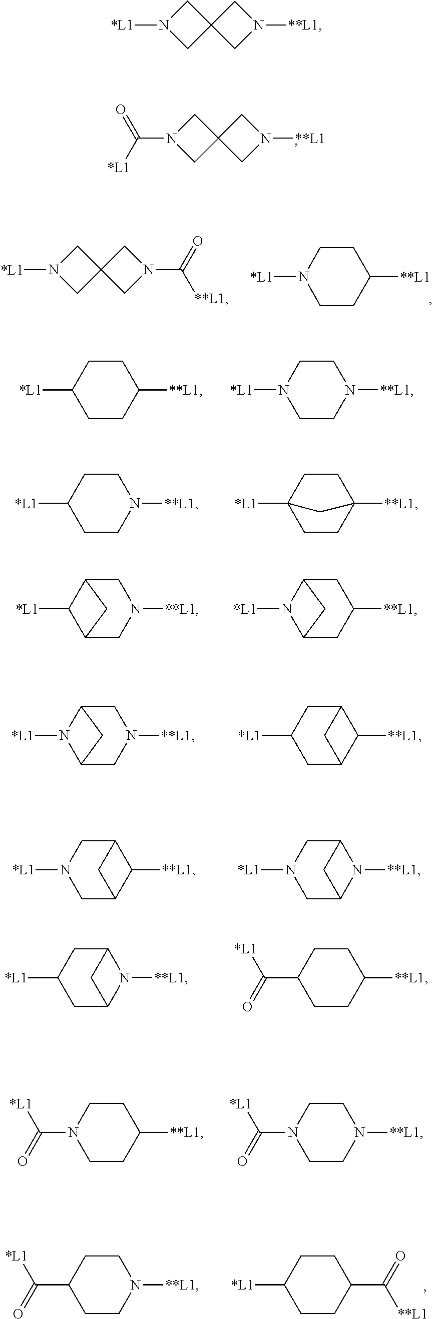

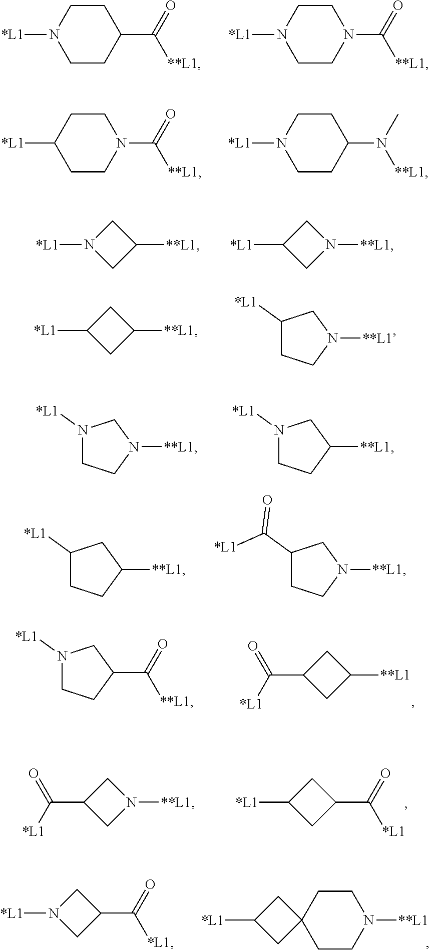

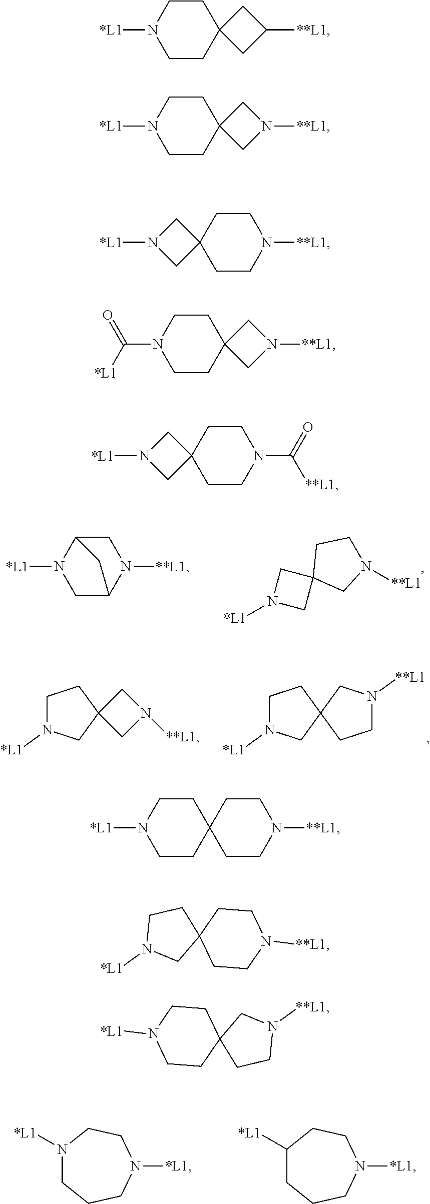

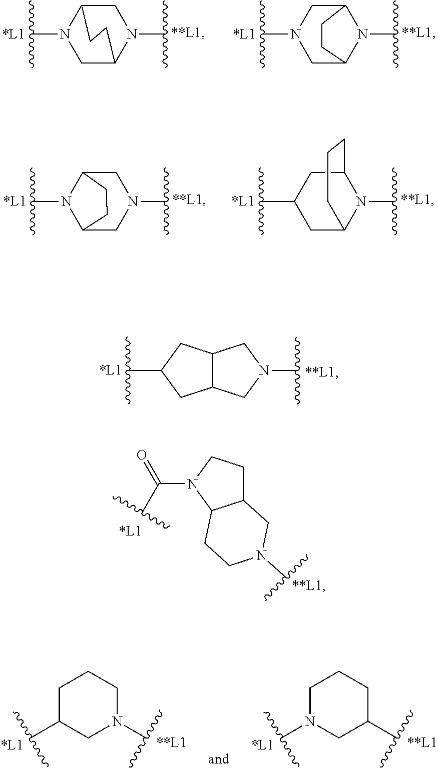

is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 21. The compound of any one of the preceding aspects, wherein L¹ is selected from —O—, —N(CH₃)—, —C(O)—, —NH—, *^{L1}—C(O)N(CH₃)—**^{L1}, *^{L1}—C(O)NH—**^{L1}, *^{L1}—C(O)O—**^{L1}, *^{L1}—C(O)N(C₂H₈)—**^{L1}, *^{L1}—C(O)N(C₃H₇)—**^{L1}, *^{L1}—N(CH₃)C(O)—**^{L1}, *^{L1}—NHC(O)—**^{L1}, *^{L1}—OC(O)—**^{L1},

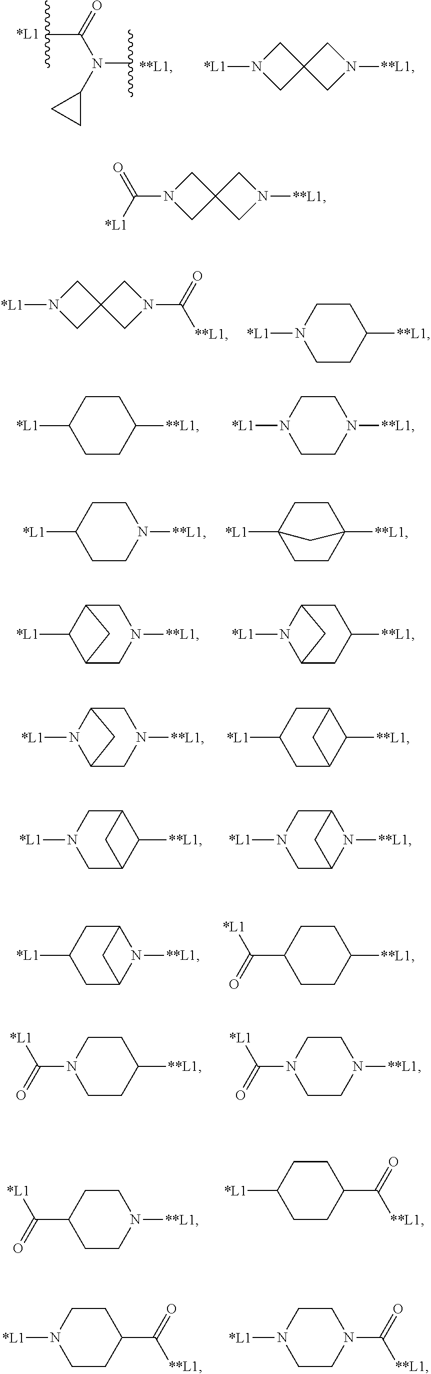

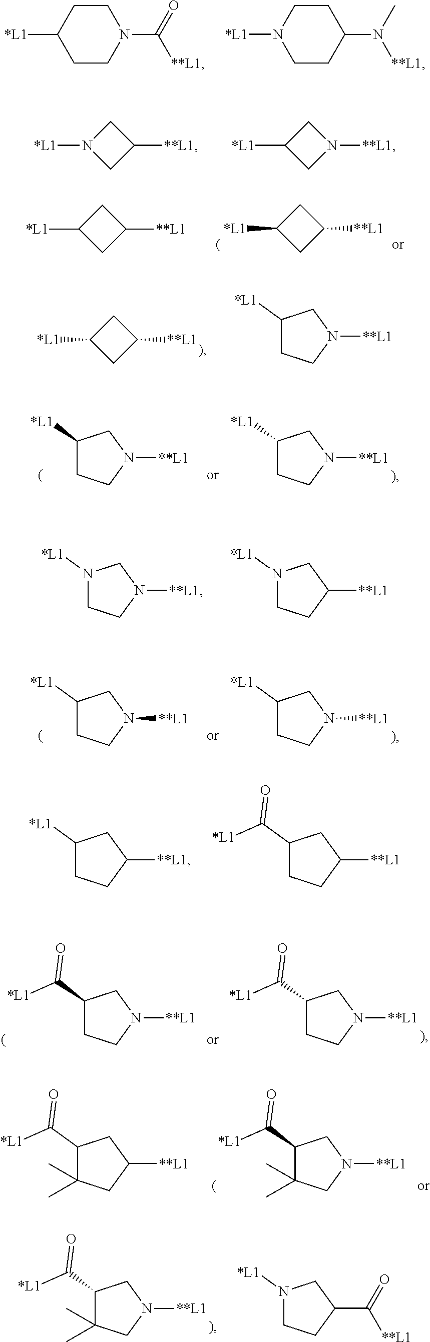

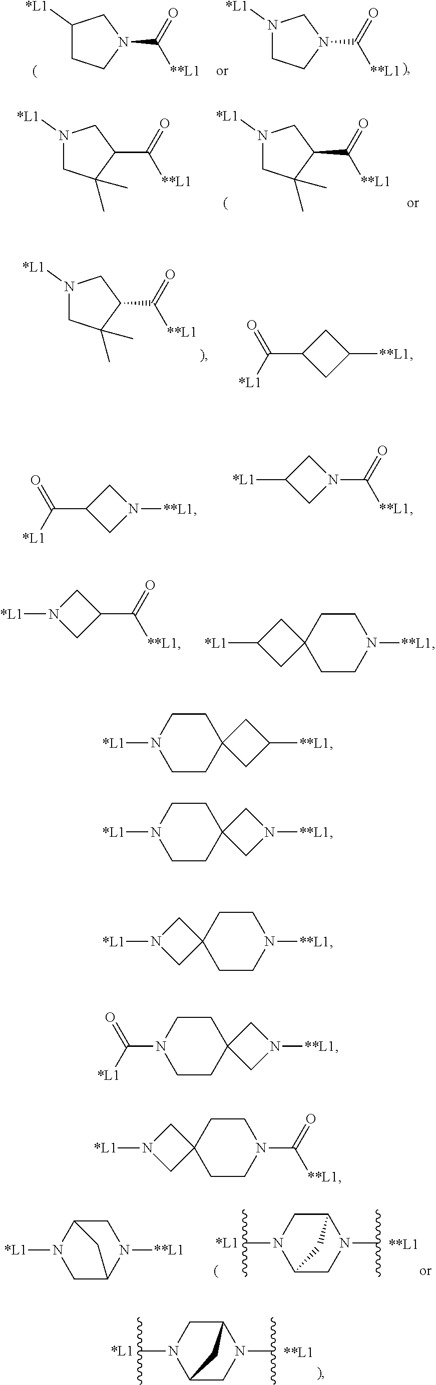

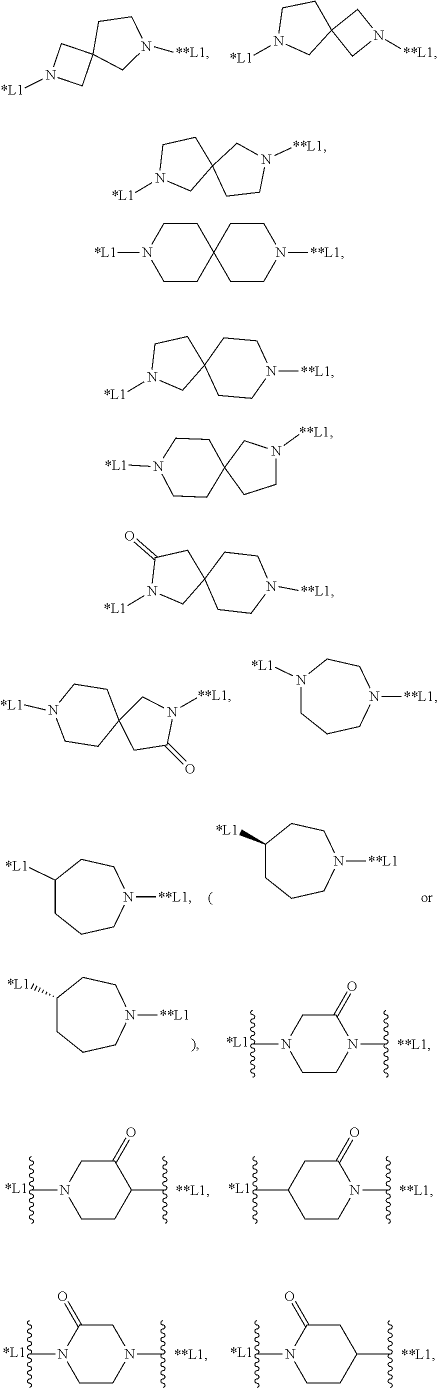

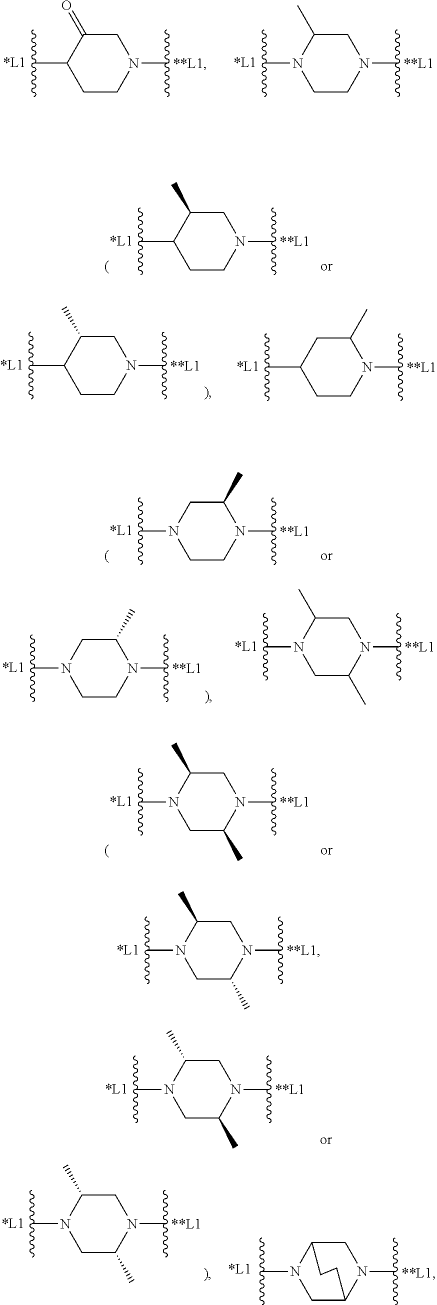

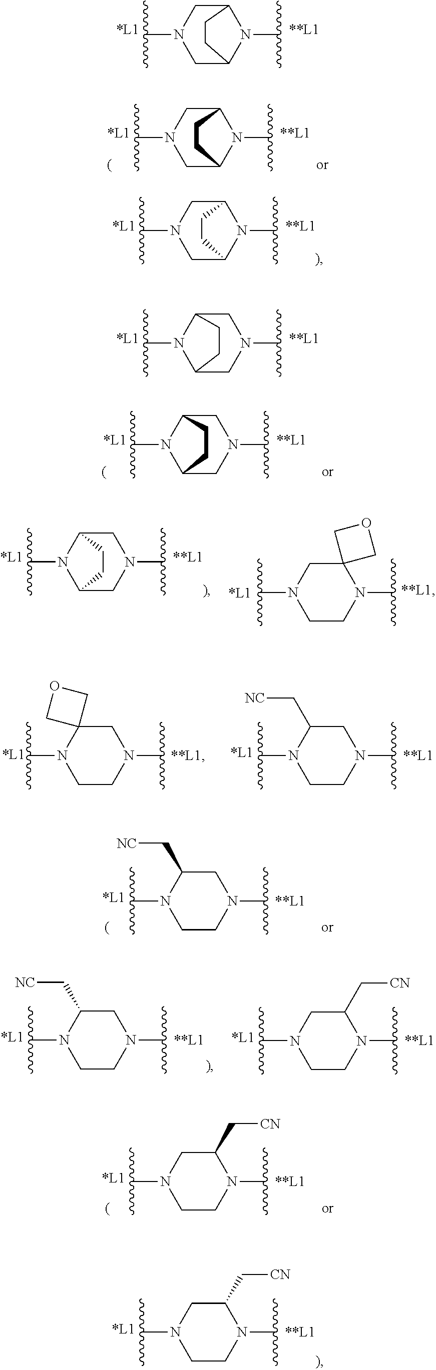

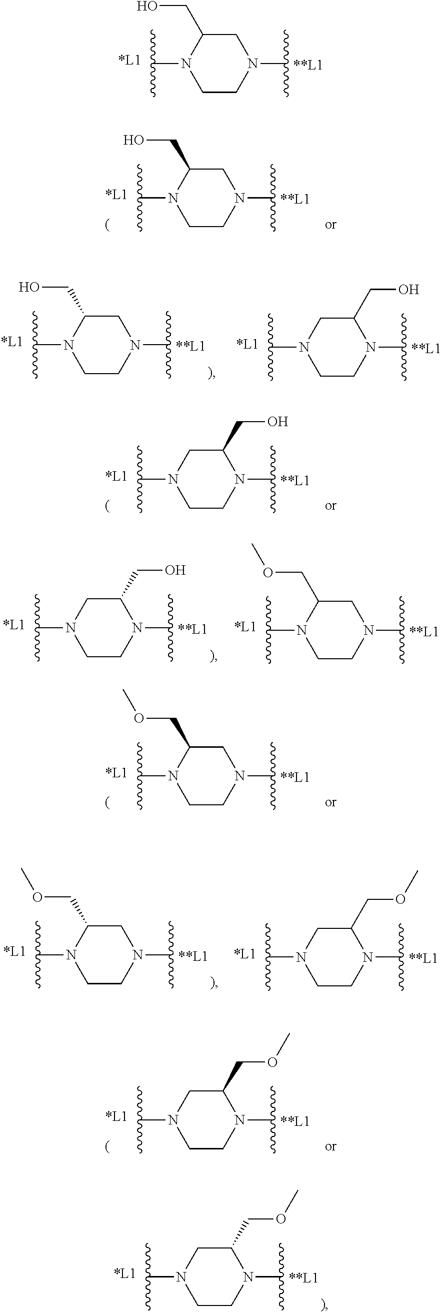

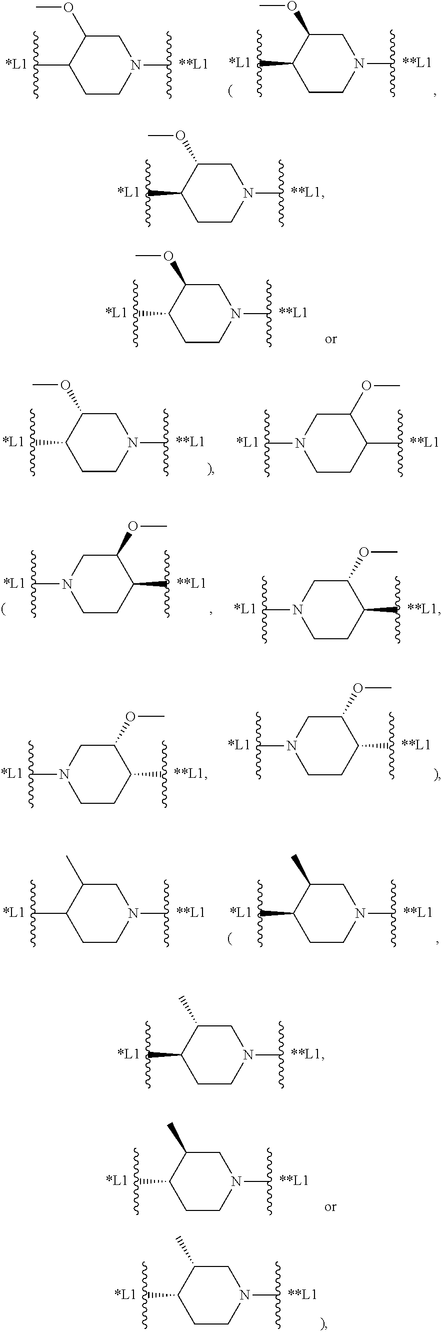

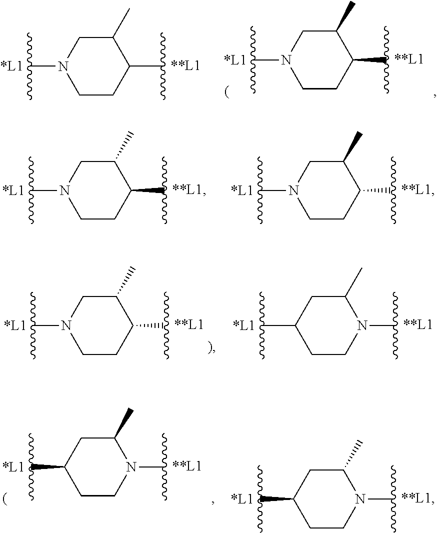

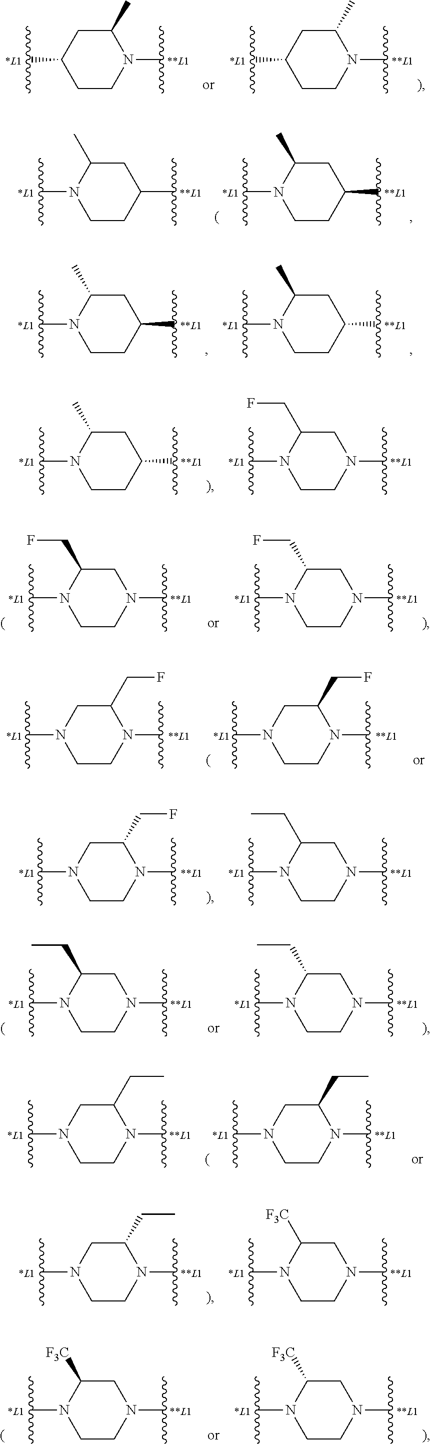

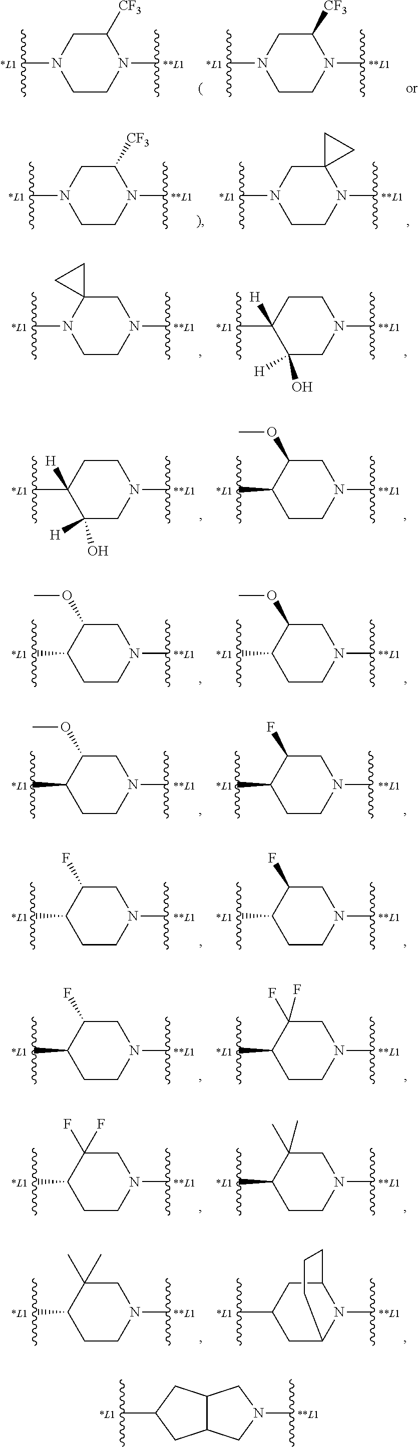

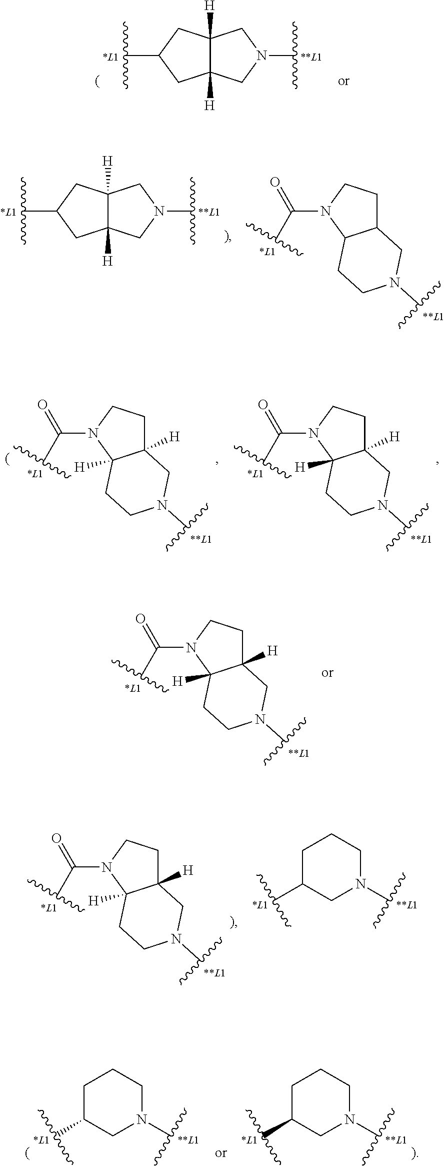

The compound of any one of the preceding aspects, wherein L² is selected from —O—, —C(O)—, —N(R^{a})—, *^{L2}—C(O)N(R^{a})—**^{L2}, *^{L2}C(O)O—**^{L2}, *^{L2}—N(R^{a})C(O)—**^{L2}, *^{L2}—OC(O)—**^{L2},

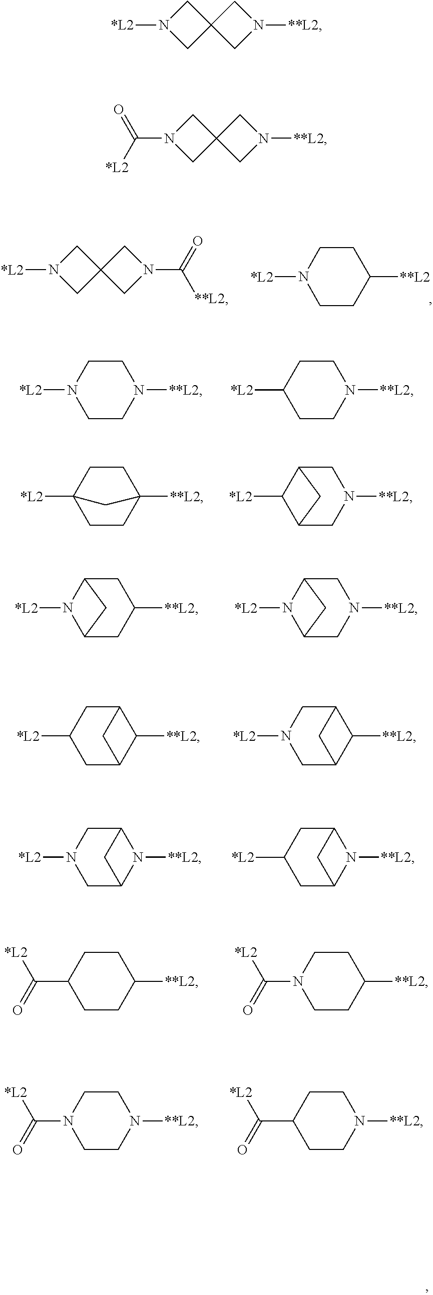

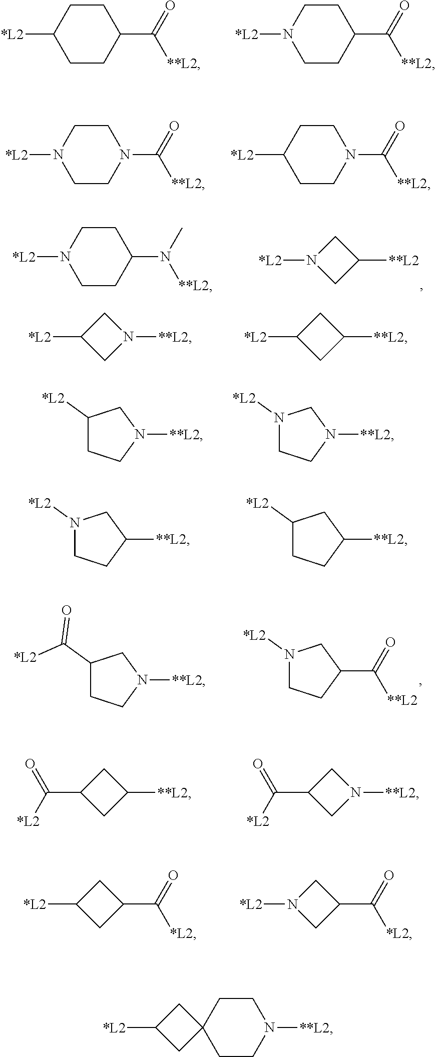

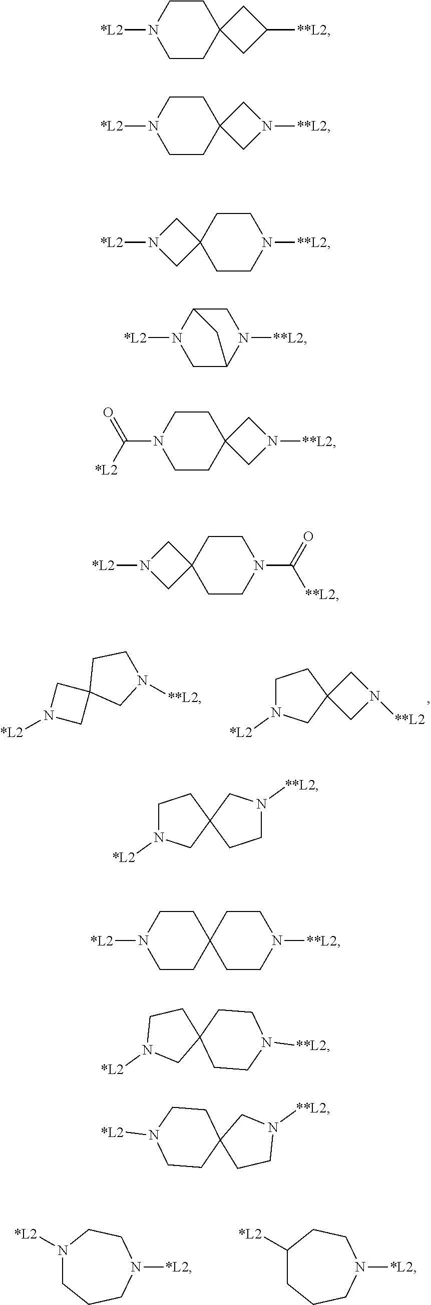

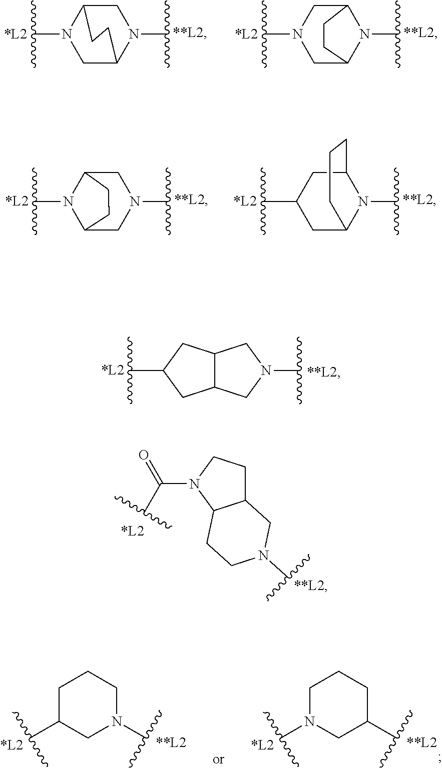

wherein each of said

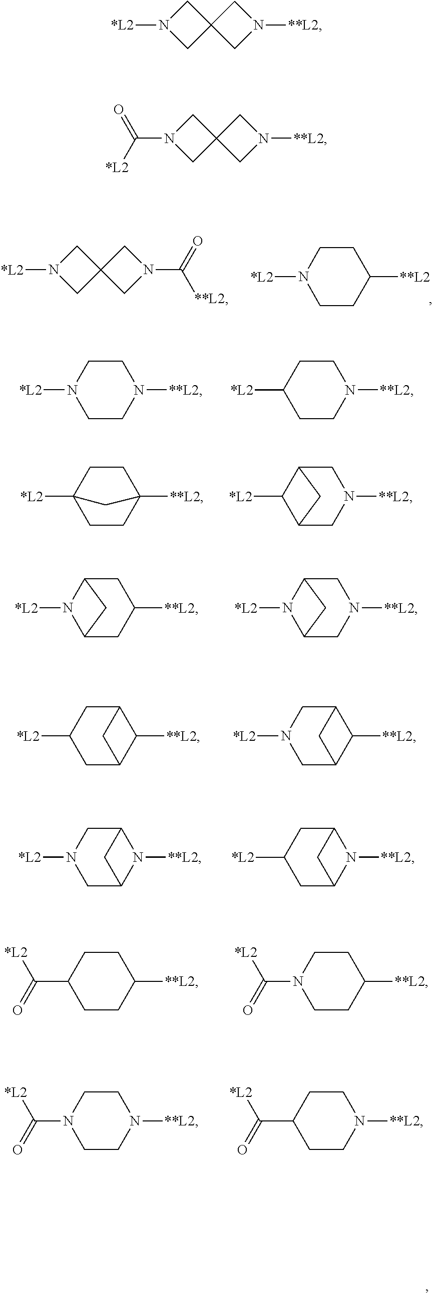

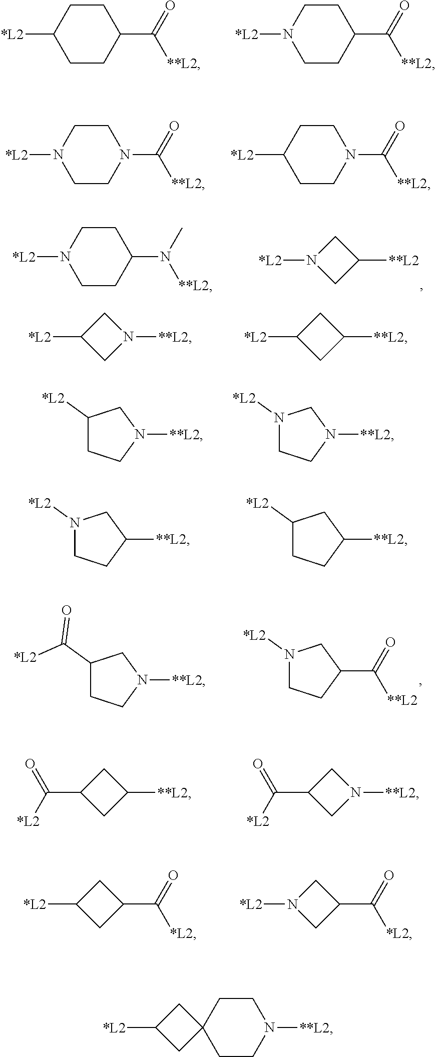

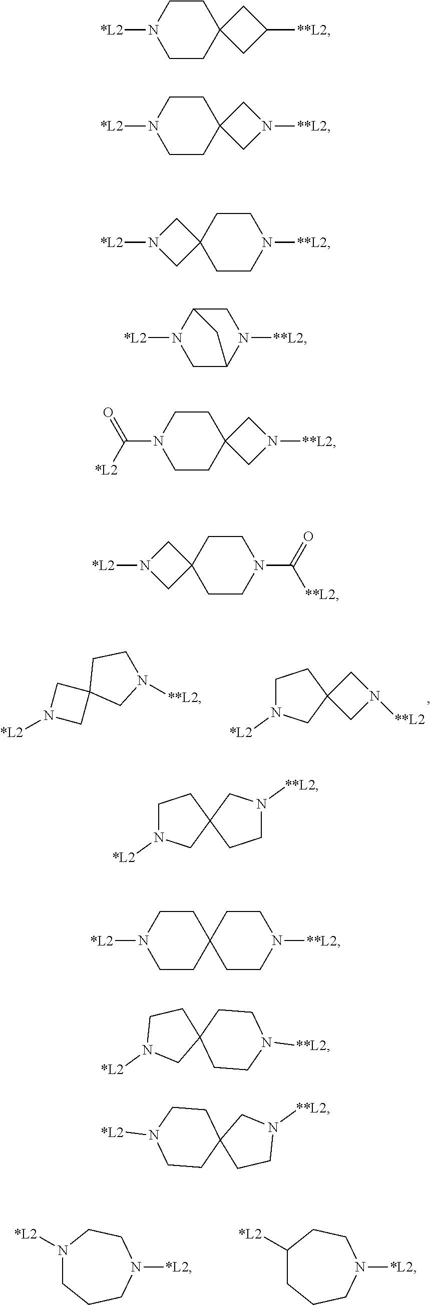

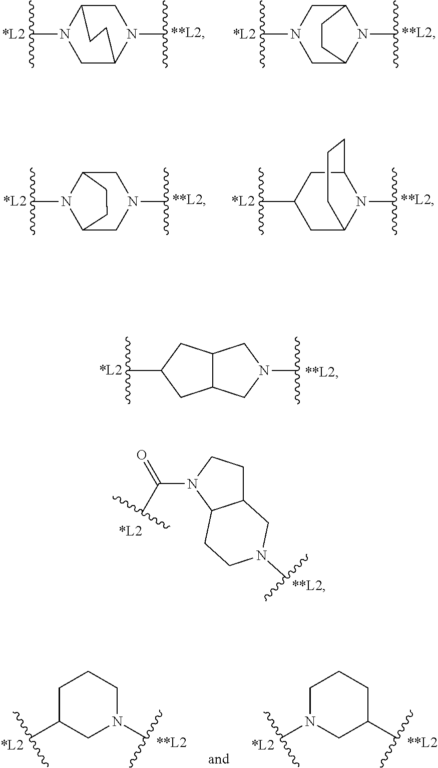

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 23. The compound of any one of the preceding aspects, wherein L² is selected from —O—, —N(CH₃)—, —C(O)—, —NH—, *^{L2}—C(O)N(CH₃)—**^{L2}, *^{L2}—C(O)NH—**^{L2}, *^{L2}—C(O)O—**^{L2}, *^{L2}—C(O)N(C₂H₅)—**^{L2}, *^{L2}—C(O)N(C₃H₇)—**^{L2}, *^{L2}—N(CH₃)C(O)—**^{L2}, *^{L2}—NHC(O)—**^{L2}, *^{L2}—OC(O)—**^{L2}, *^{L2}—N(C₂H₅)C(O)—**^{L2}, *^{L2}—N(C₃H₇)C(O)—**^{L2},

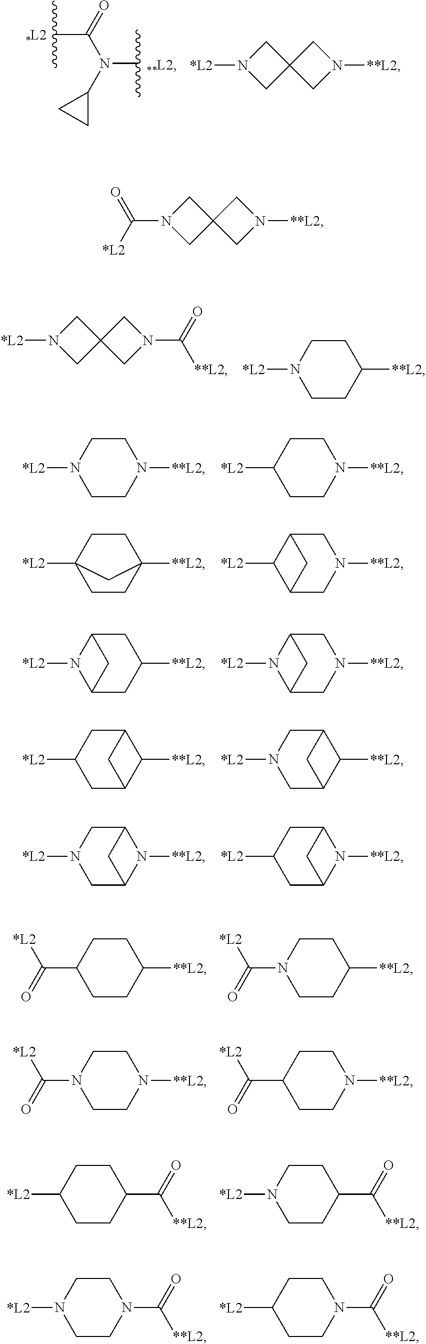

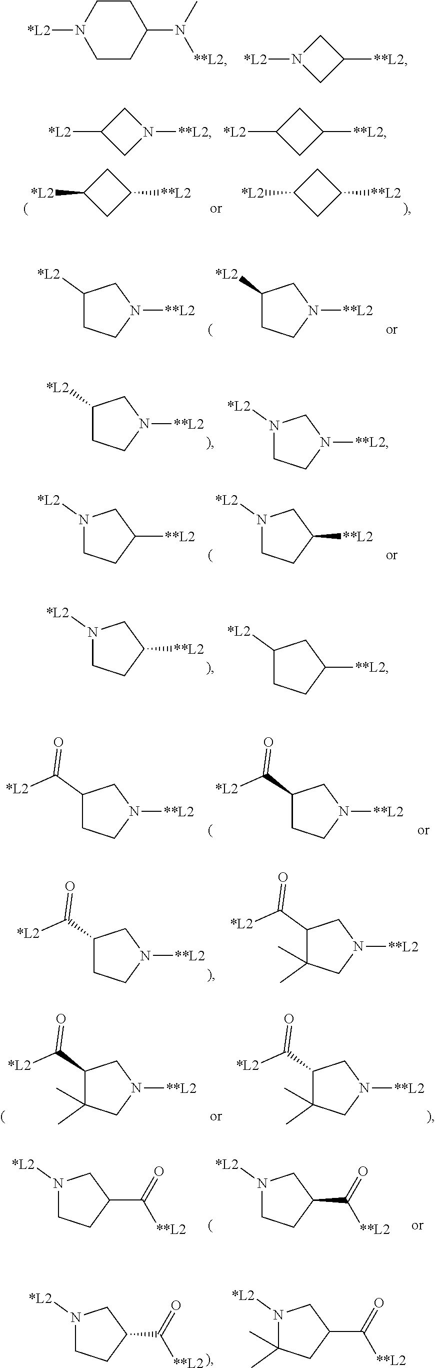

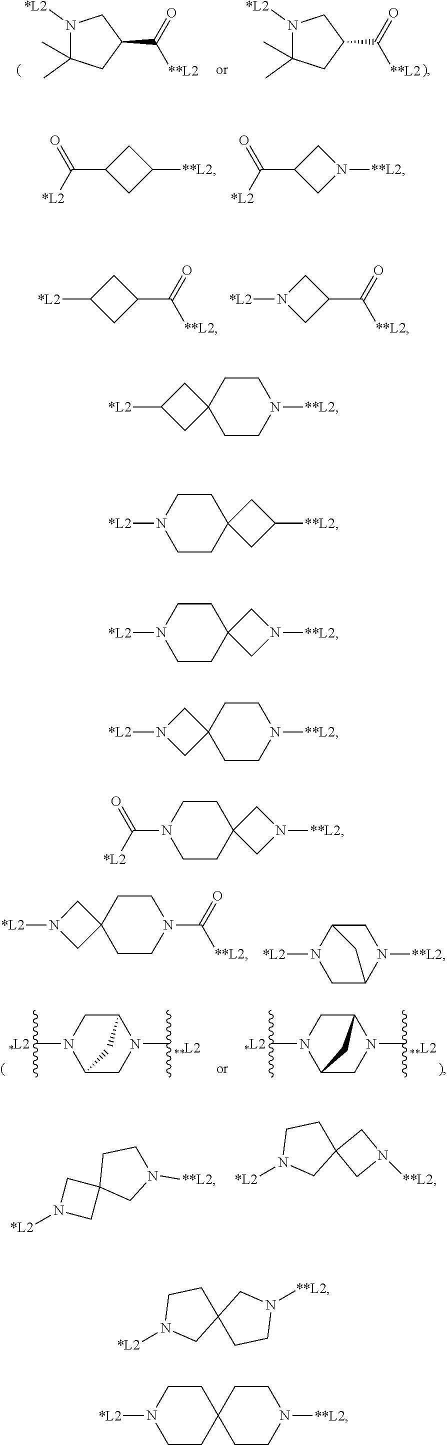

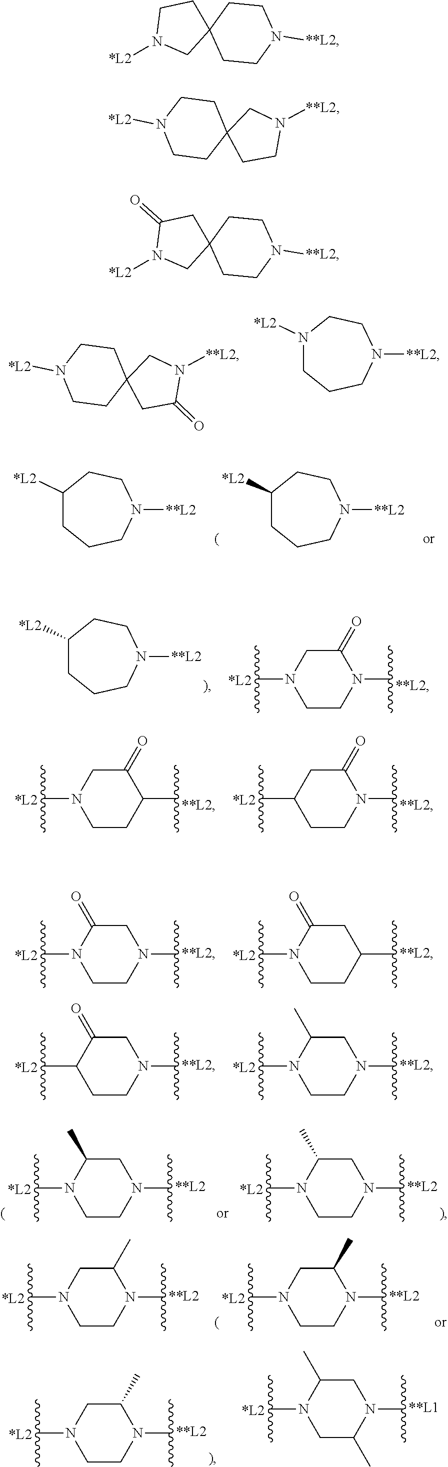

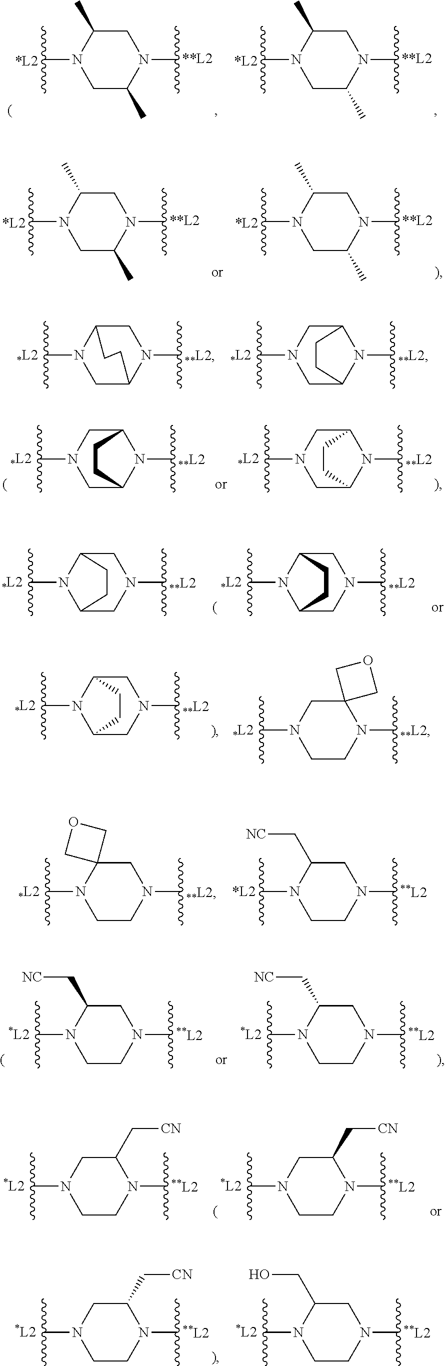

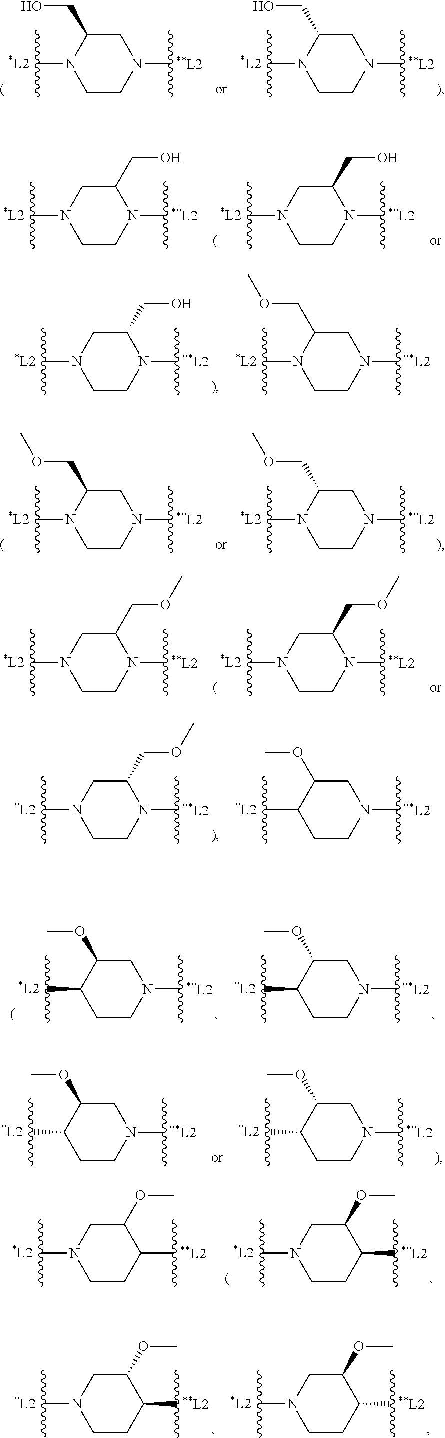

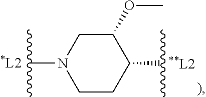 [SMILES: *[C@@H]1CCN(*)C[C@@H]1OC]

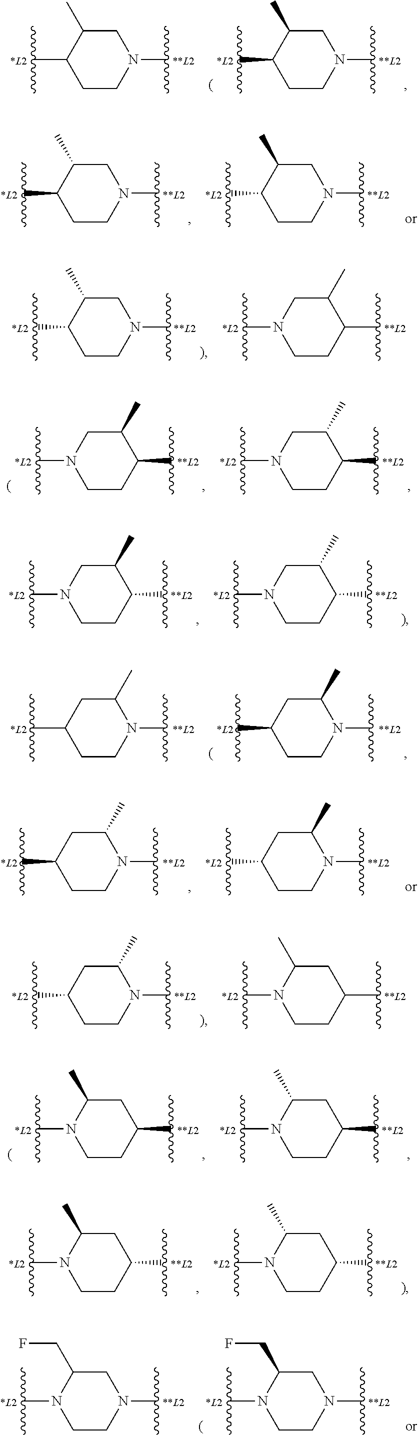

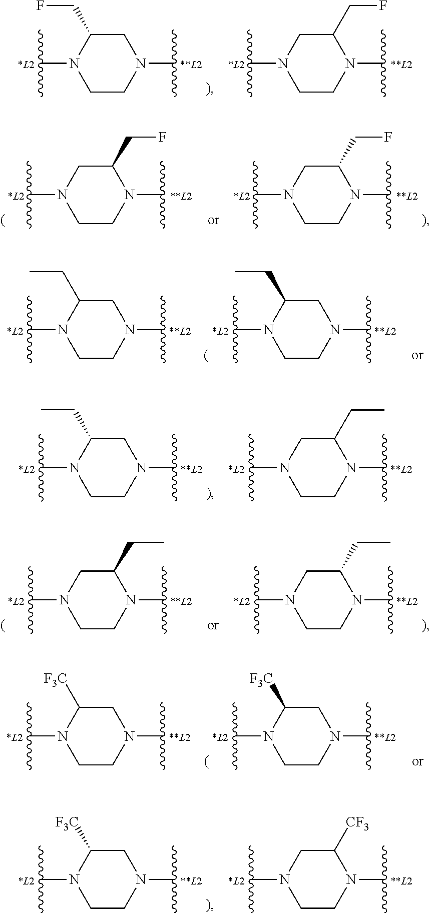

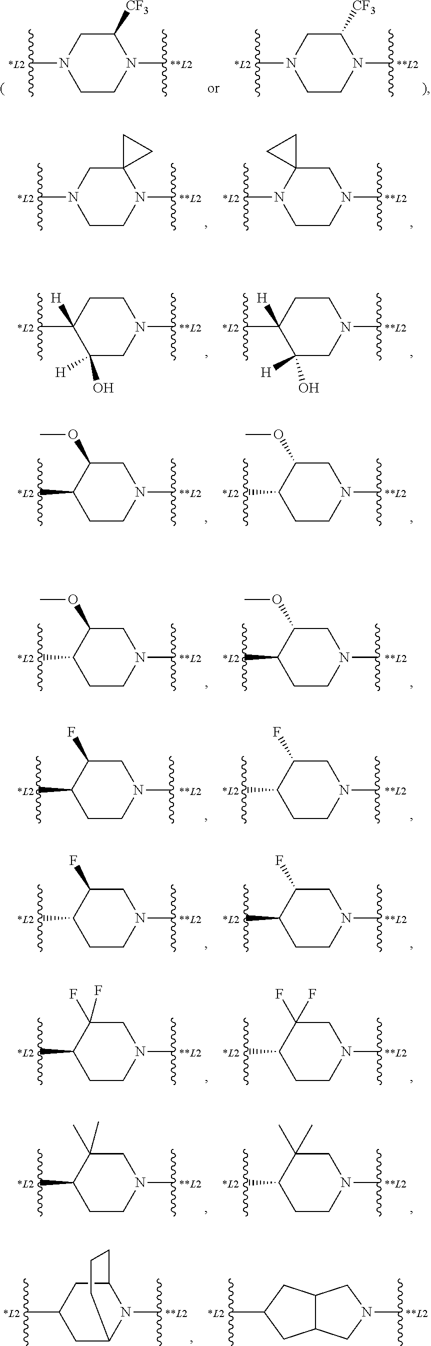

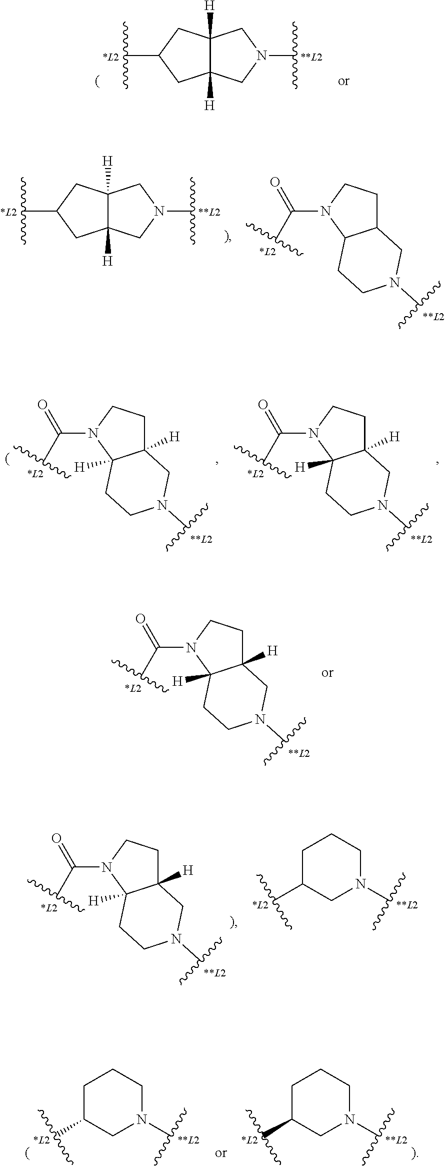

Aspect 24. The compound of any one of the preceding aspects, wherein L³ is selected from —O—, —N(R^{a})—, —C(O)—, *^{L3}—C(O)N(R^{a})—**^{L3}, *^{L3}—C(O)O—**^{L3}, *^{L3}—N(R^{a})C(O)—**^{L3}, *^{L3}—OC(O)**^{L3},

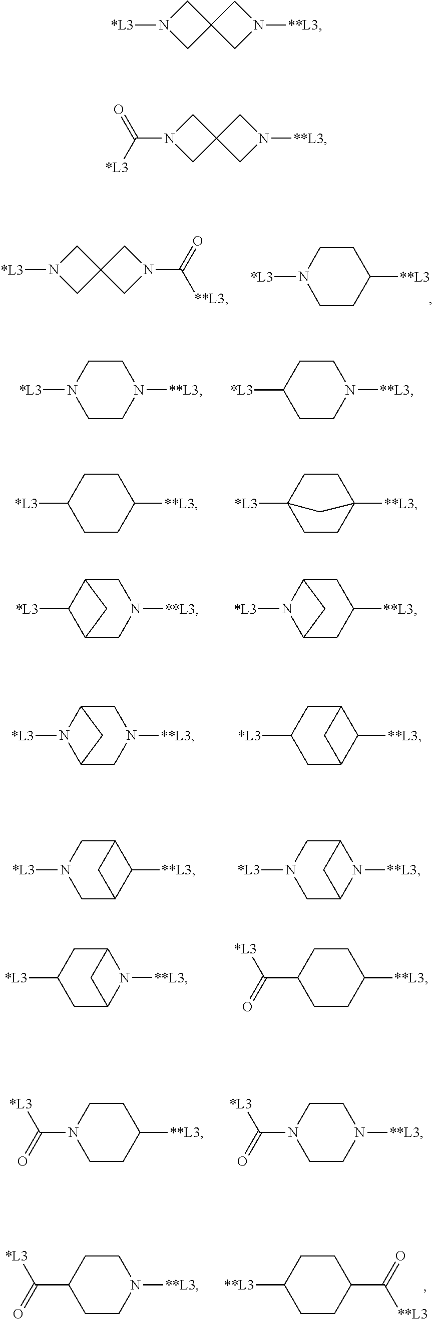

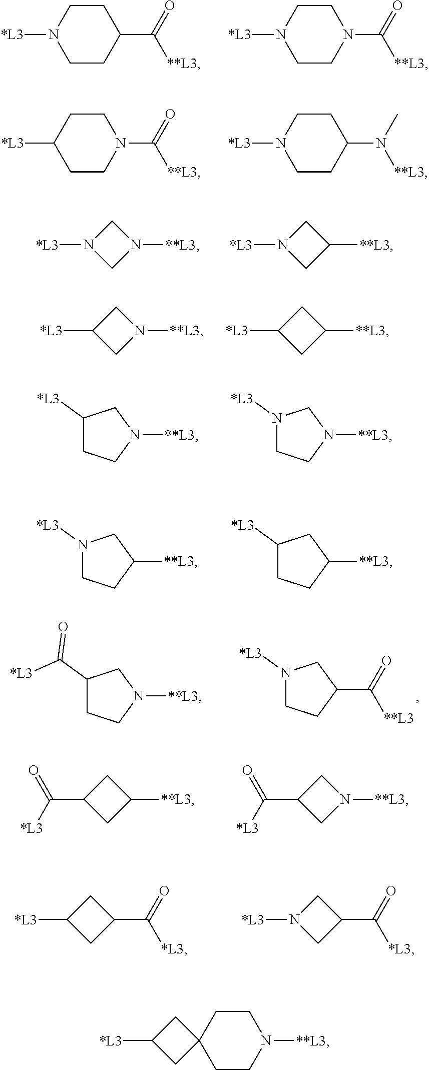

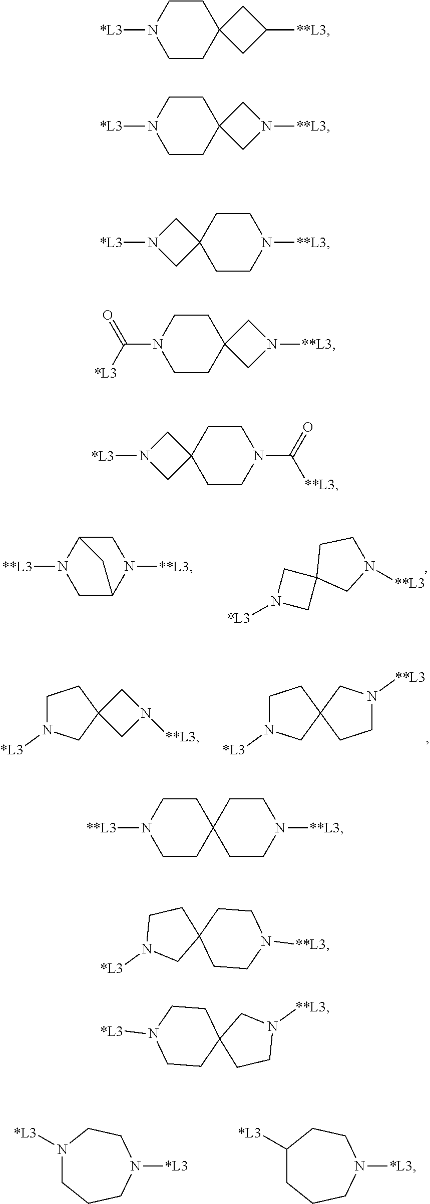

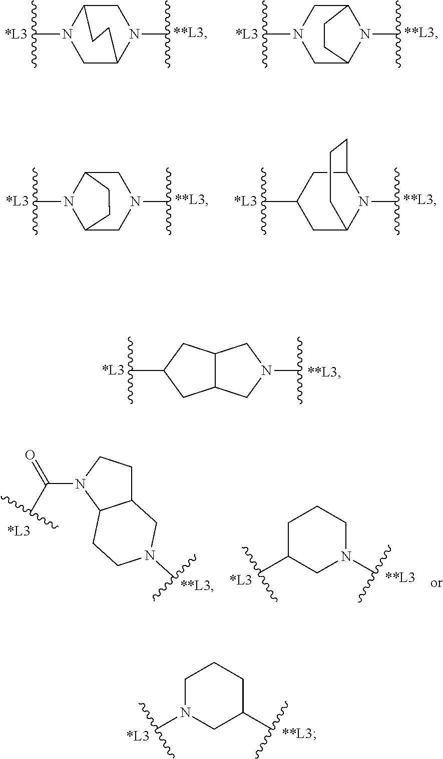

wherein each of said

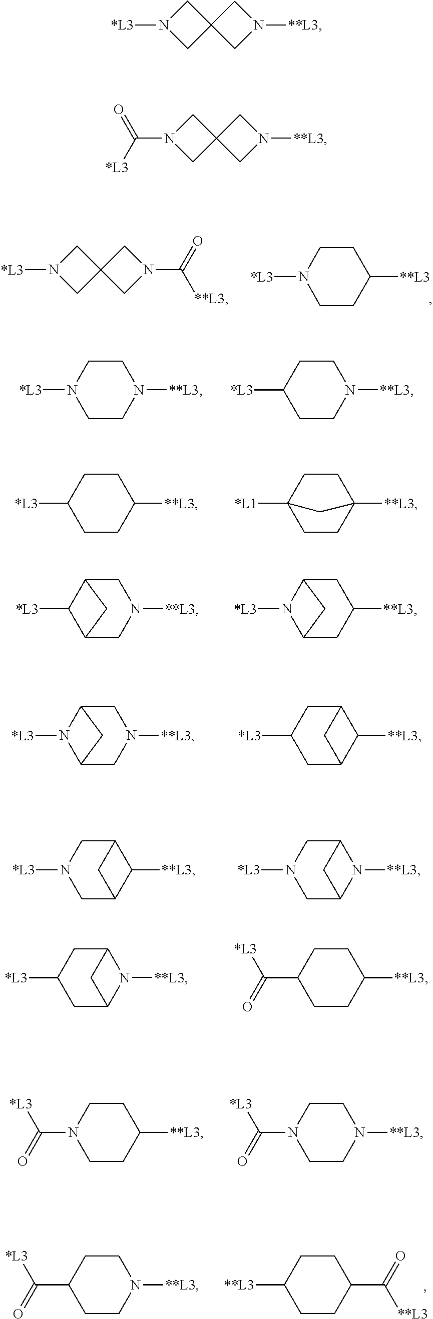

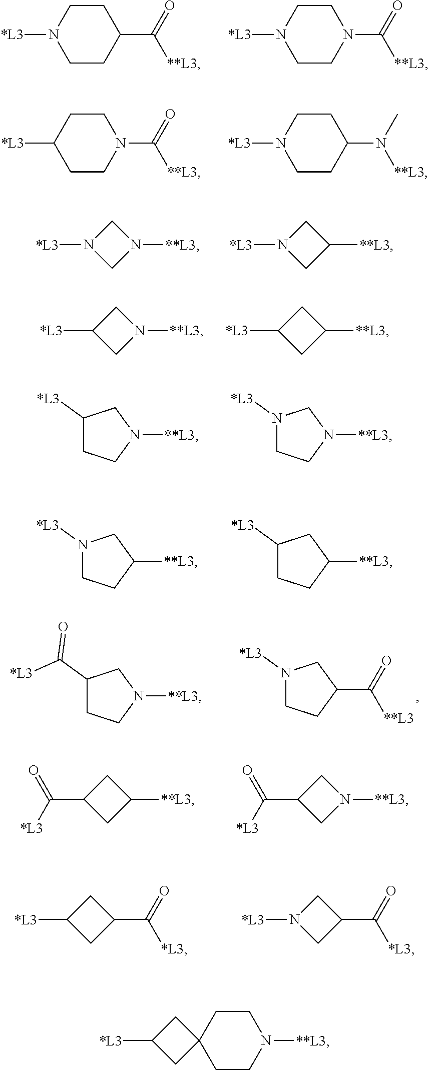

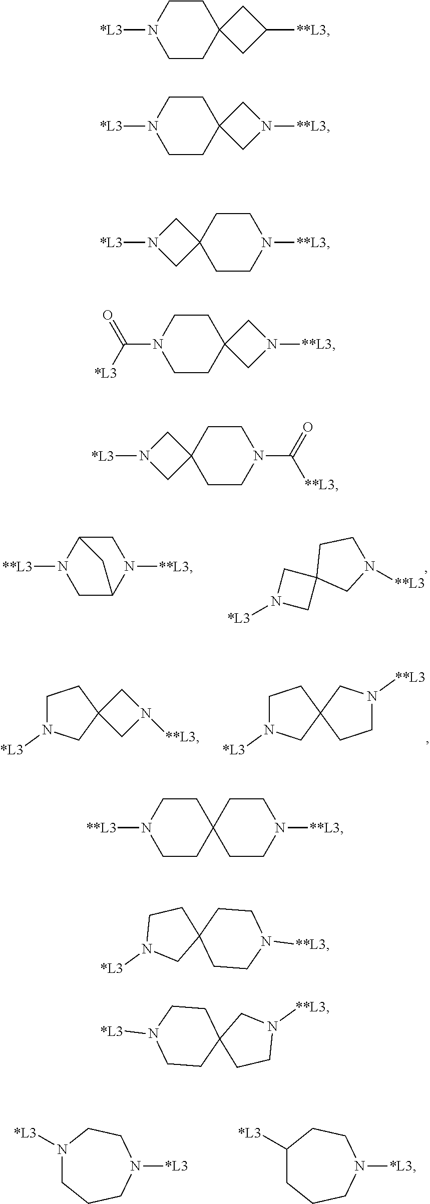

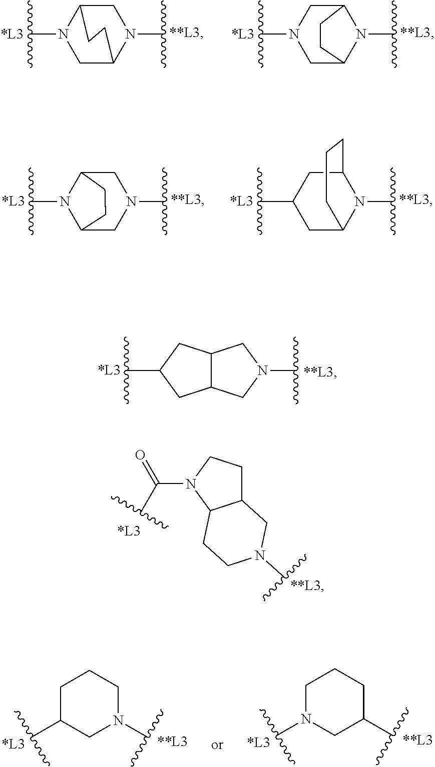

is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur: said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 25. The compound of any one of the preceding aspects, wherein L³ is selected from —O—, —N(CH₃)—, —C(O)—, —NH—, *^{L3}—C(O)N(CH₃)—**^{L1}, *^{L3}—C(O)NH—**^{L3}, *^{L3}—C(O)O—**^{L3}, *^{L3}—C(O)N(C₂H₈)—**^{L3}, *^{L3}—C(O)N(C₃H₇)—**^{L3}, *^{L3}—N(CH₃)C(O)—**^{L3}, *^{L3}—NHC(O)—**^{L3}, *^{L3}—OC(O)—**^{L3},

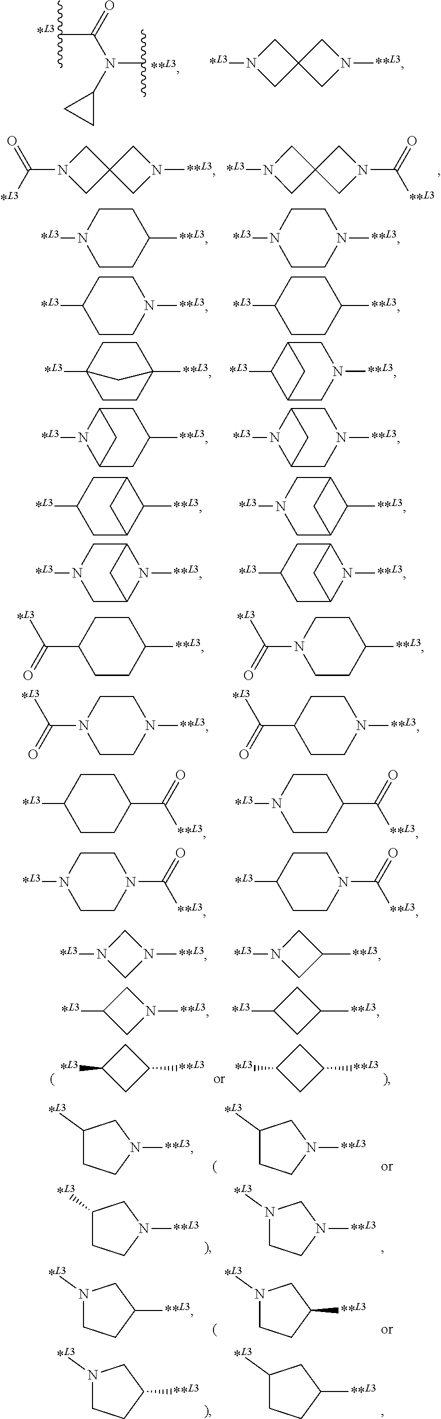

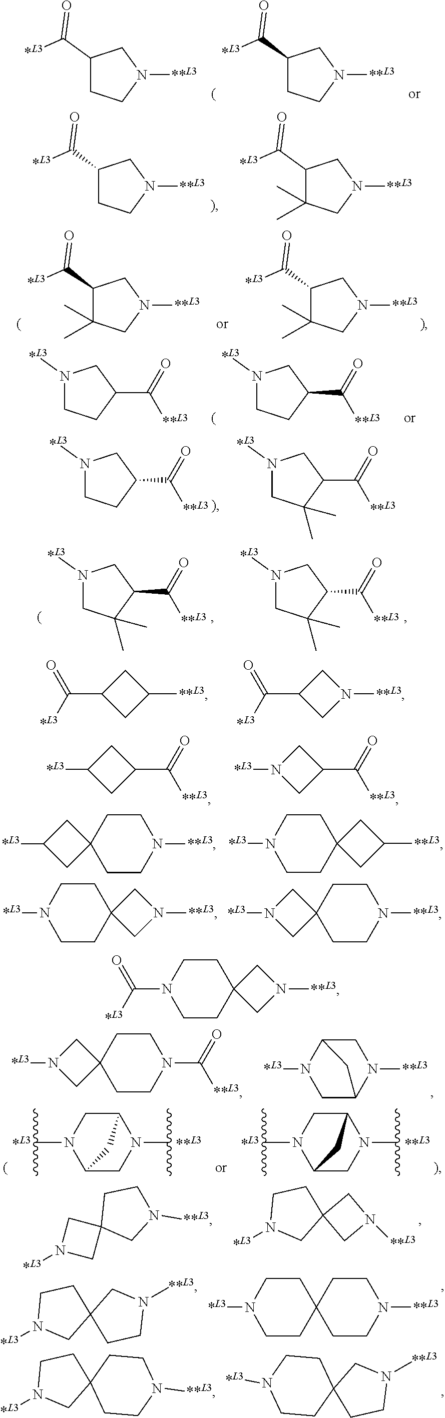

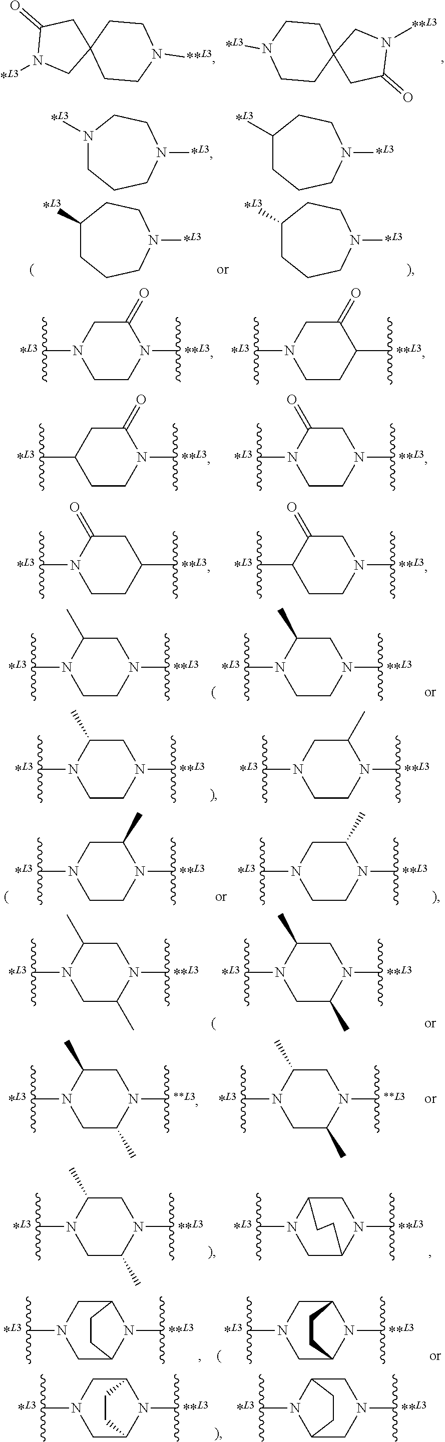

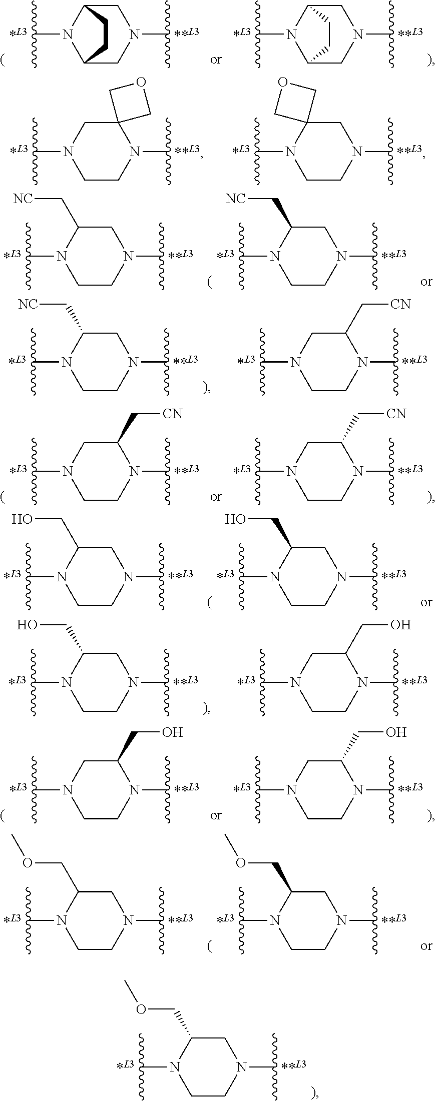

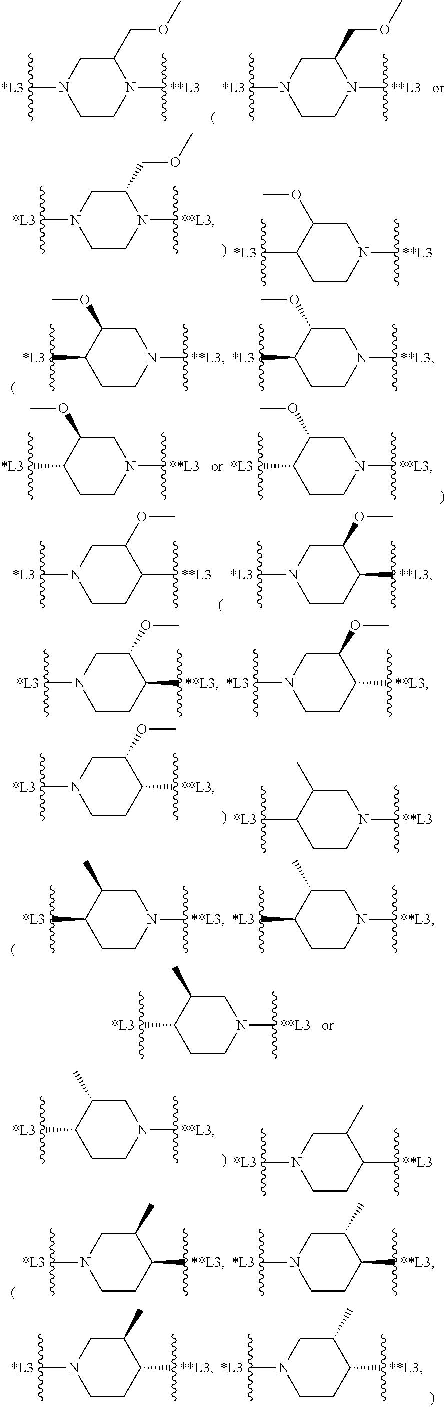

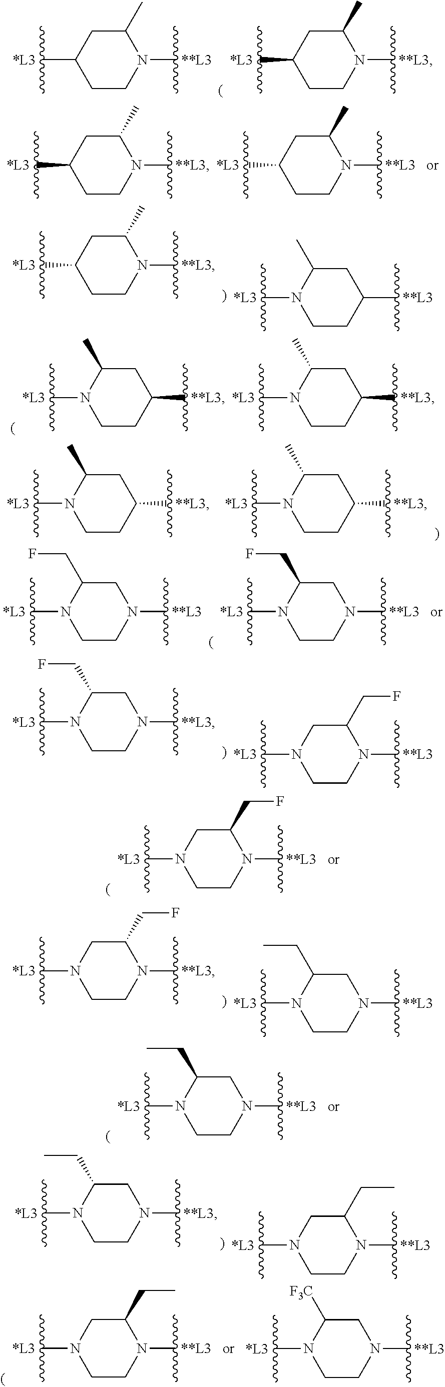

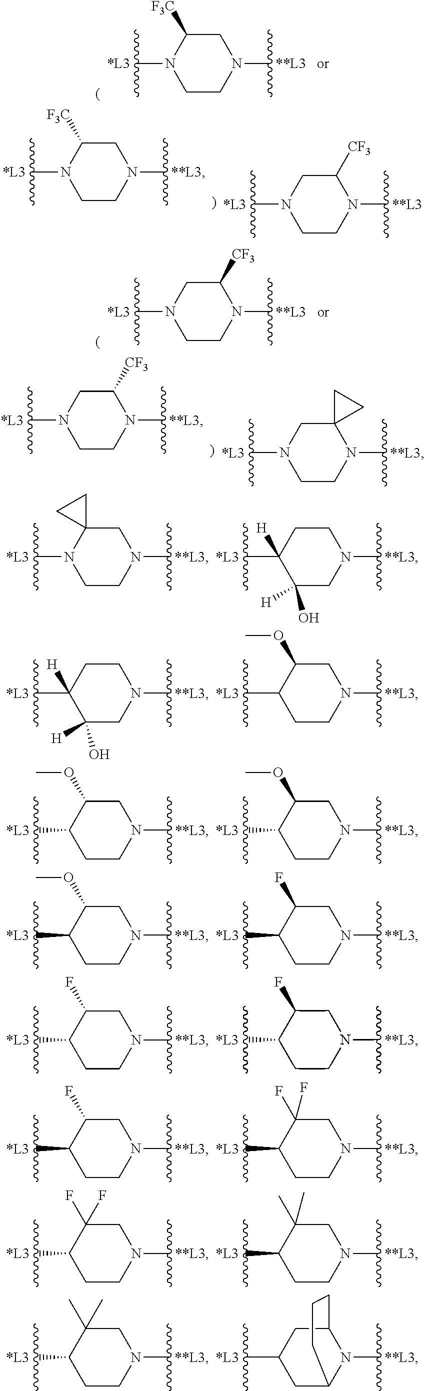

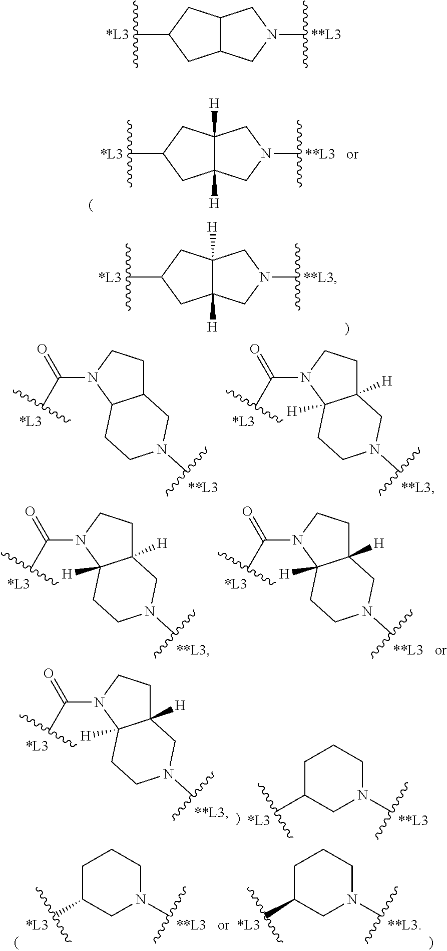

Aspect 26. The compound of any one of the preceding aspects, wherein

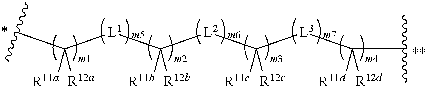

moiety is selected from

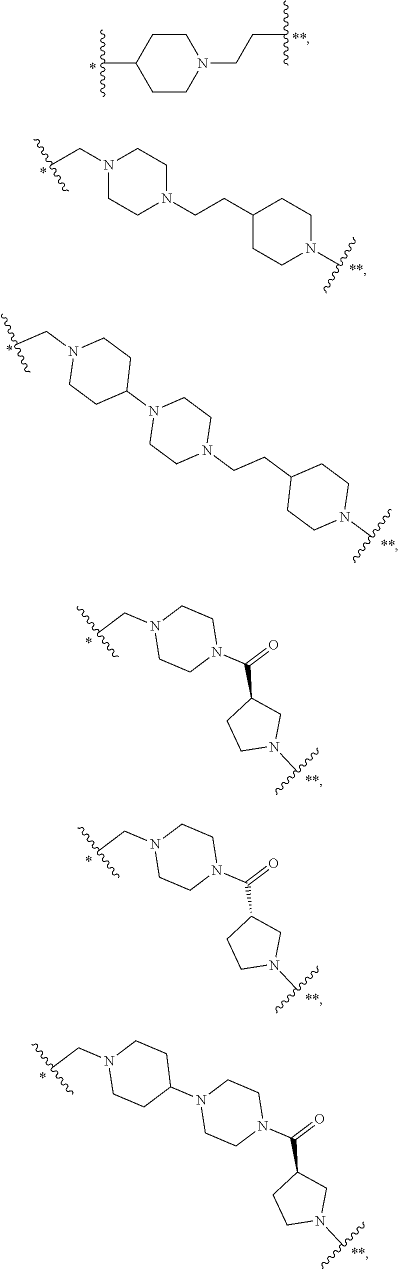 [SMILES: *CCN1CCC(*)CC1 ; *CN1CCN(CCC2CCN(*)CC2)CC1 ; *CN1CCC(N2CCN(CCC3CCN(*)CC3)CC2)CC1 ; *CN1CCN(C(=O)[C@@H]2CCN(*)C2)CC1 ; *CN1CCN(C(=O)[C@H]2CCN(*)C2)CC1 ; *CN1CCC(N2CCN(C(=O)[C@@H]3CCN(*)C3)CC2)CC1]

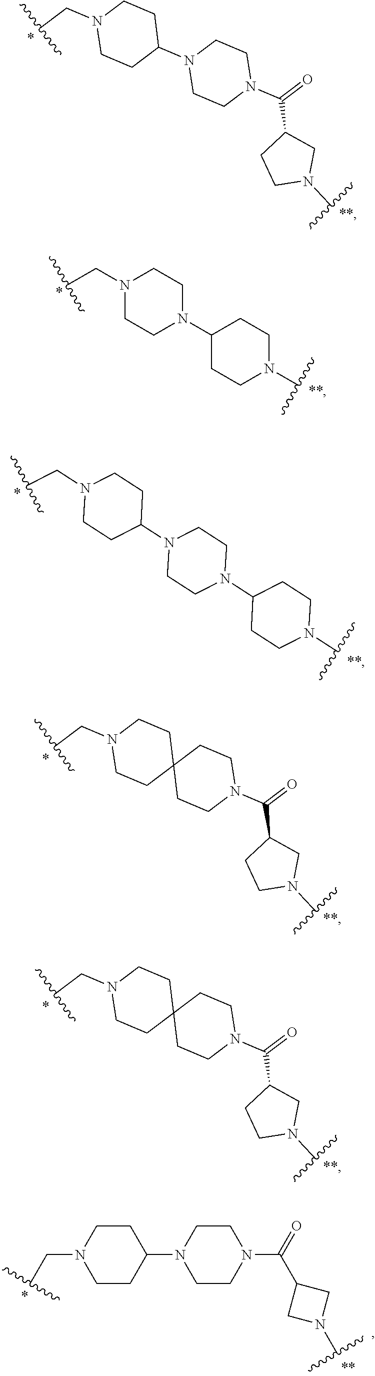 [SMILES: *CN1CCC(N2CCN(C(=O)[C@H]3CCN(*)C3)CC2)CC1 ; *CN1CCN(C2CCN(*)CC2)CC1 ; *CN1CCC(N2CCN(C3CCN(*)CC3)CC2)CC1 ; *CN1CCC2(CC1)CCN(C(=O)[C@@H]1CCN(*)C1)CC2 ; *CN1CCC2(CC1)CCN(C(=O)[C@H]1CCN(*)C1)CC2 ; *CN1CCC(N2CCN(C(=O)C3CN(*)C3)CC2)CC1]

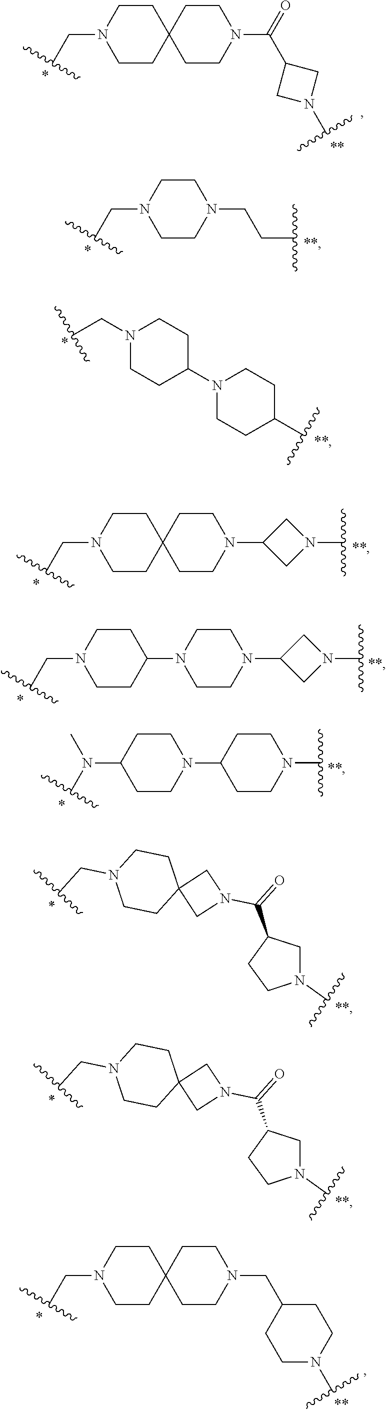

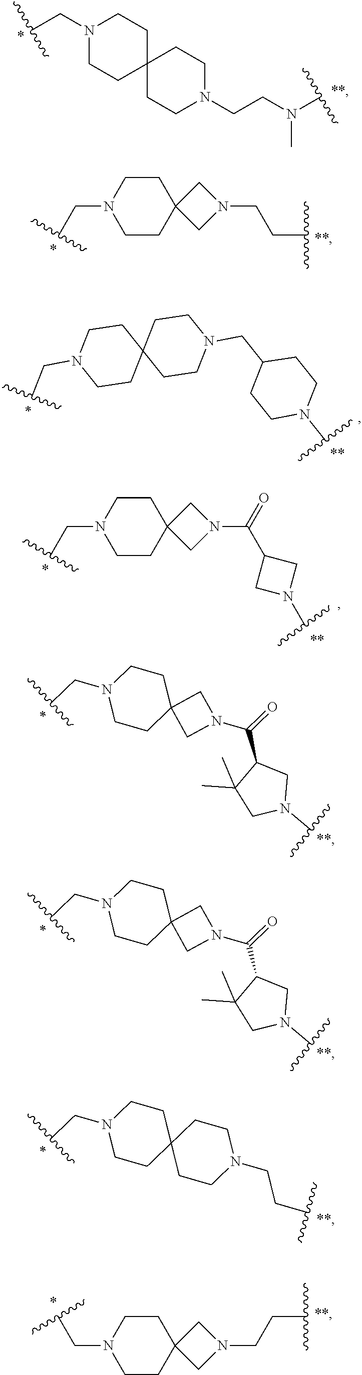

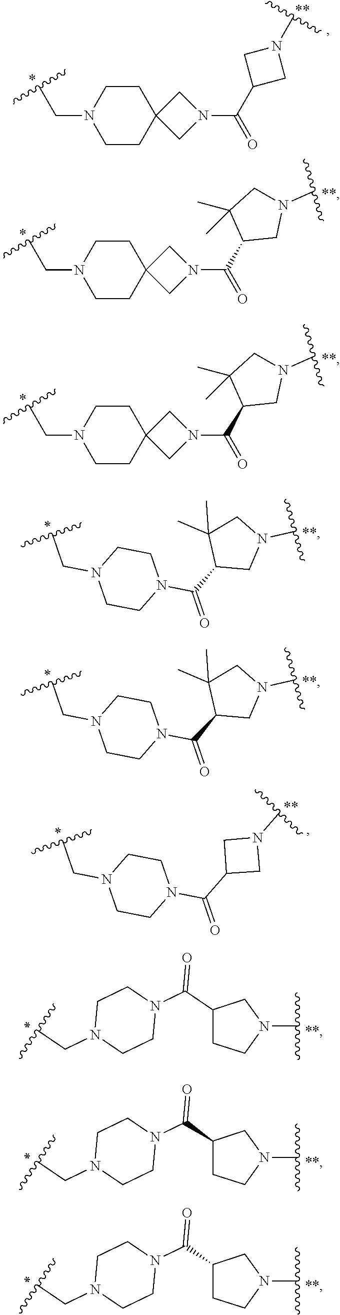

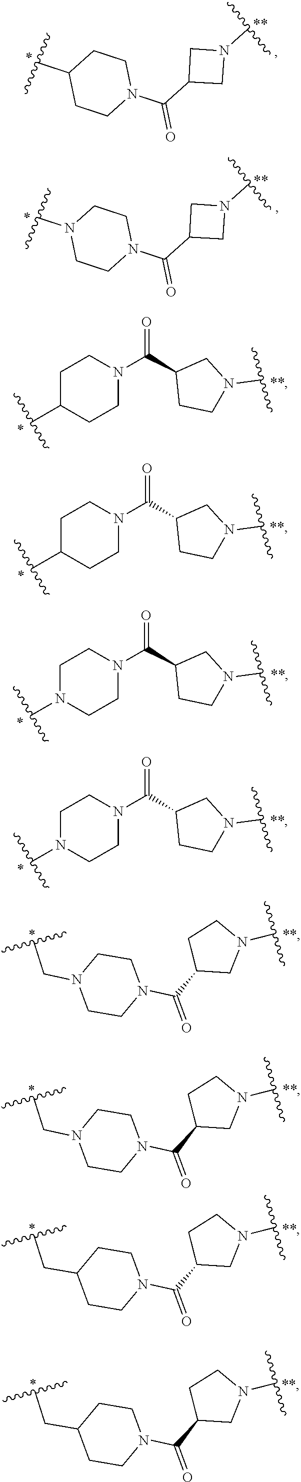

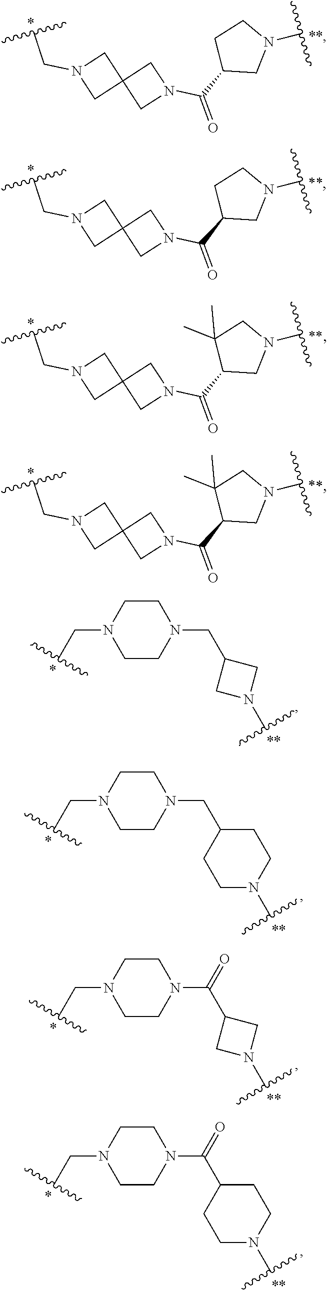

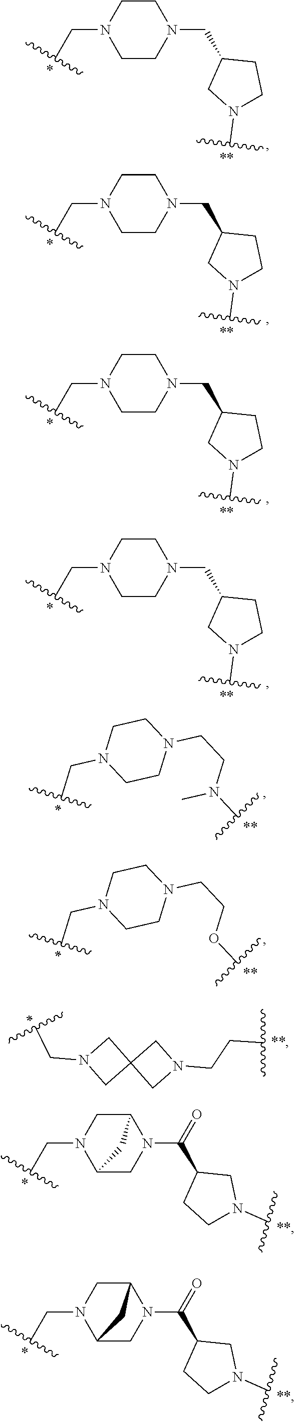

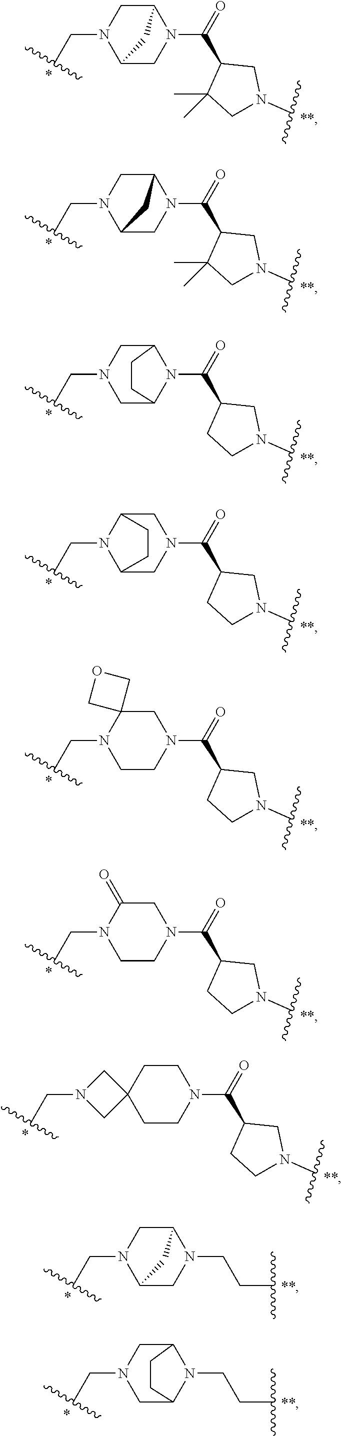

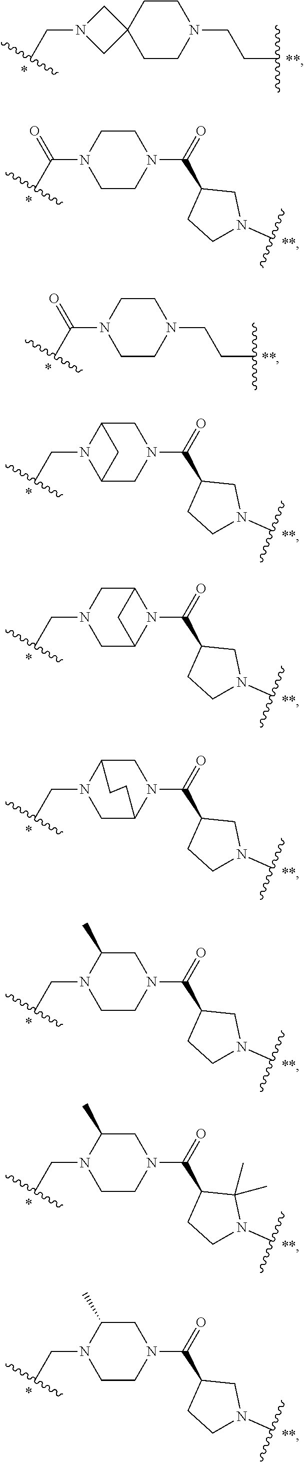

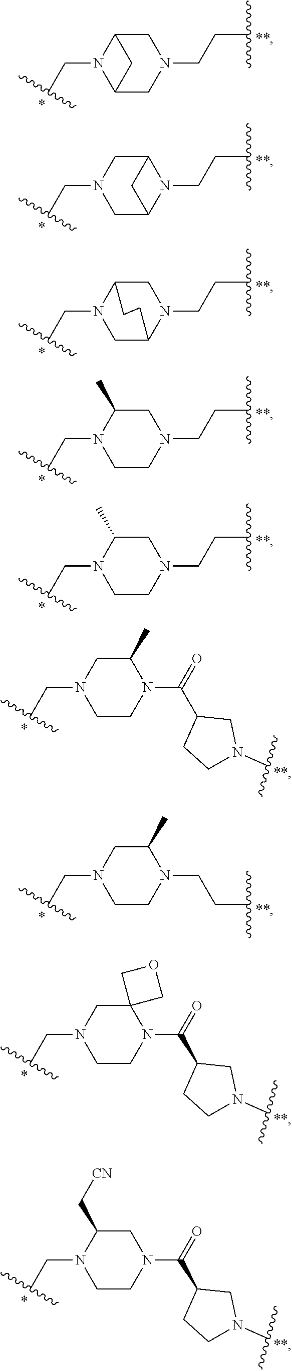

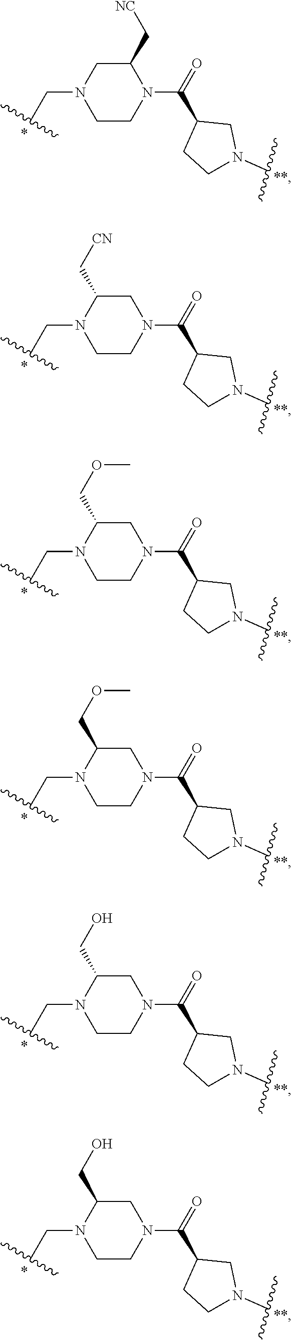 [SMILES: *CN1CCN(C(=O)[C@@H]2CCN(*)C2)[C@H](CC#N)C1 ; *CN1CCN(C(=O)[C@@H]2CCN(*)C2)C[C@H]1CC#N ; *CN1CCN(C(=O)[C@@H]2CCN(*)C2)C[C@H]1COC ; *CN1CCN(C(=O)[C@@H]2CCN(*)C2)C[C@@H]1COC ; *CN1CCN(C(=O)[C@@H]2CCN(*)C2)C[C@H]1CO ; *CN1CCN(C(=O)[C@@H]2CCN(*)C2)C[C@@H]1CO]

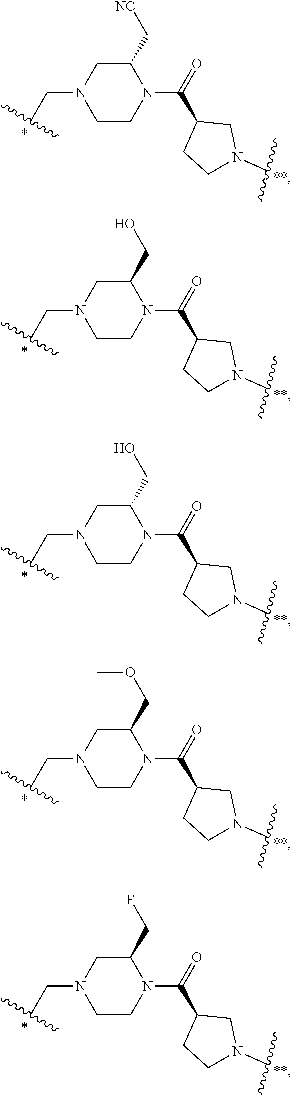 [SMILES: *CN1CCN(C(=O)[C@@H]2CCN(*)C2)[C@@H](CC#N)C1 ; *CN1CCN(C(=O)[C@@H]2CCN(*)C2)[C@H](CO)C1 ; *CN1CCN(C(=O)[C@@H]2CCN(*)C2)[C@@H](CO)C1 ; *CN1CCN(C(=O)[C@@H]2CCN(*)C2)[C@H](COC)C1 ; *CN1CCN(C(=O)[C@@H]2CCN(*)C2)[C@H](CF)C1]

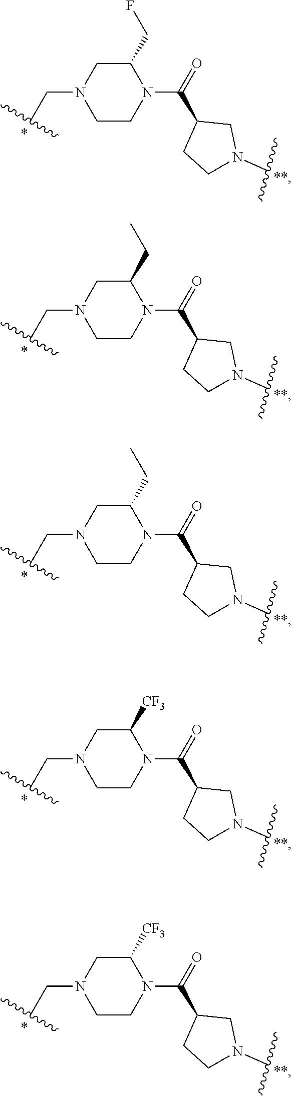 [SMILES: *CN1CCN(C(=O)[C@@H]2CCN(*)C2)[C@@H](CF)C1 ; *CN1CCN(C(=O)[C@@H]2CCN(*)C2)[C@H](CC)C1 ; *CN1CCN(C(=O)[C@@H]2CCN(*)C2)[C@@H](CC)C1 ; *CN1CCN(C(=O)[C@@H]2CCN(*)C2)[C@H](C(F)(F)F)C1 ; *CN1CCN(C(=O)[C@@H]2CCN(*)C2)[C@@H](C(F)(F)F)C1]

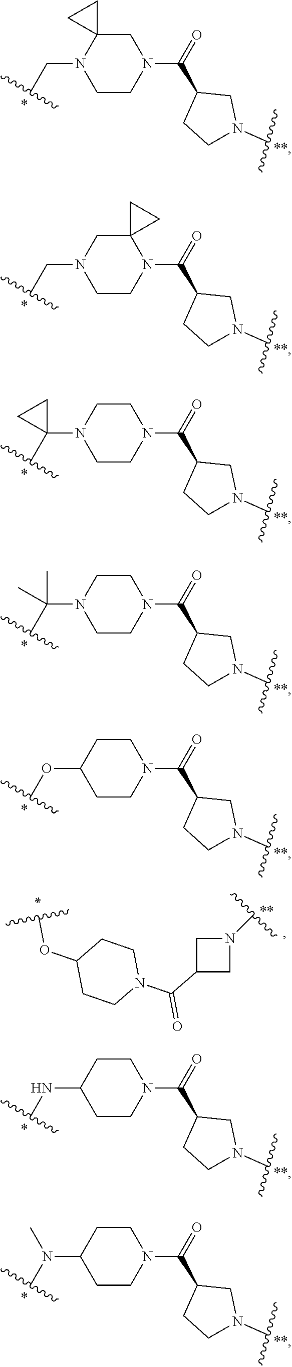

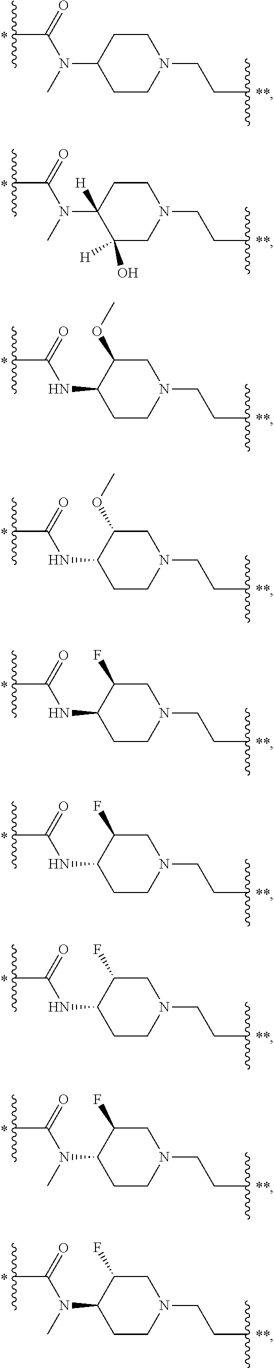

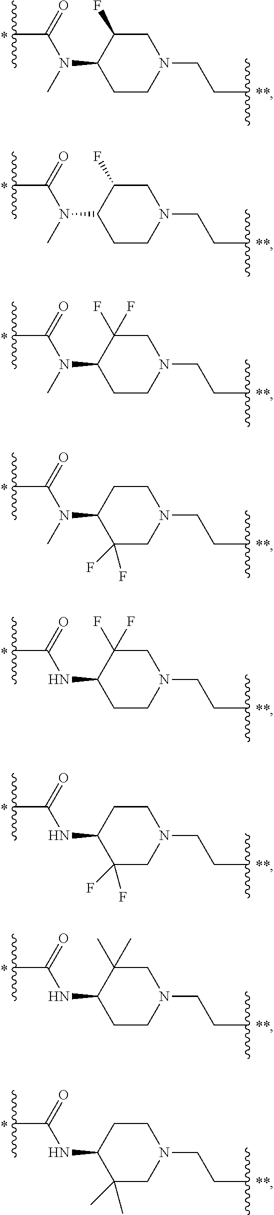

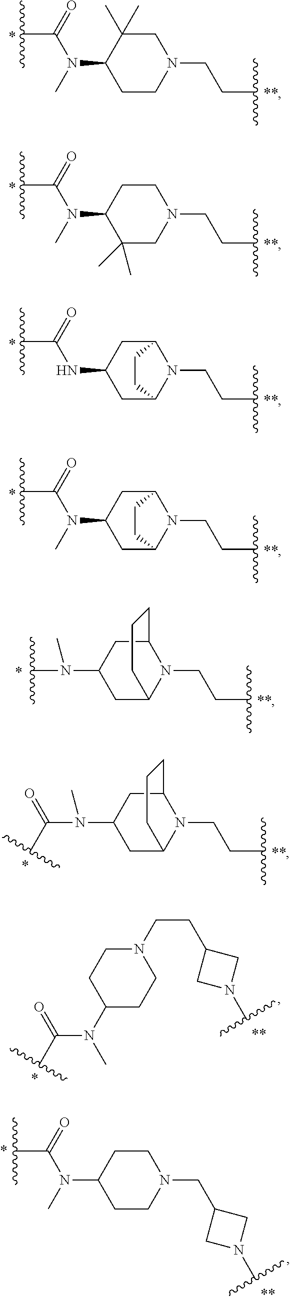

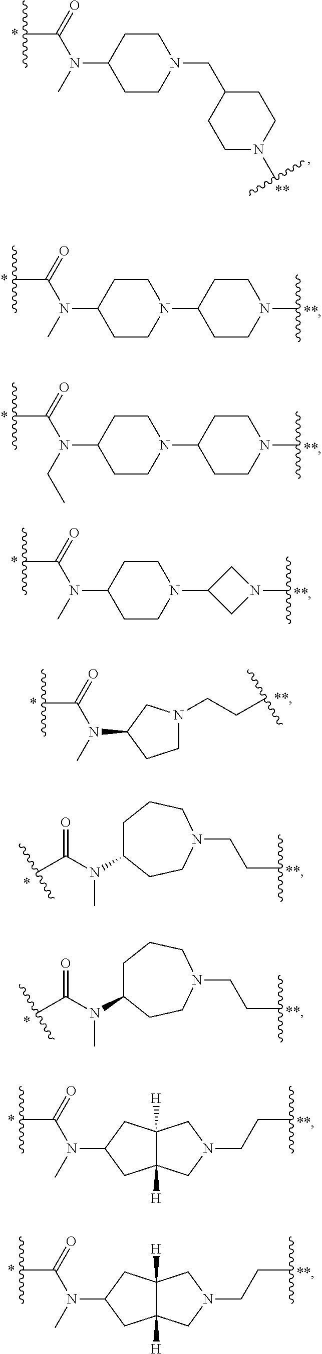

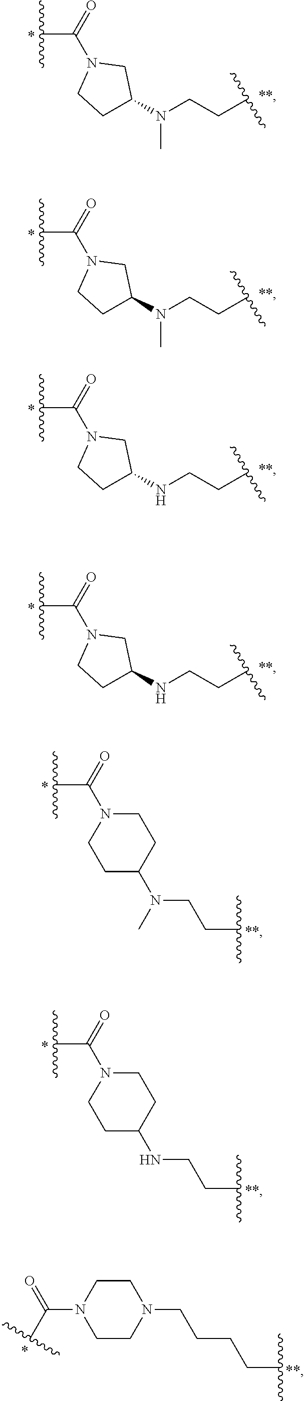

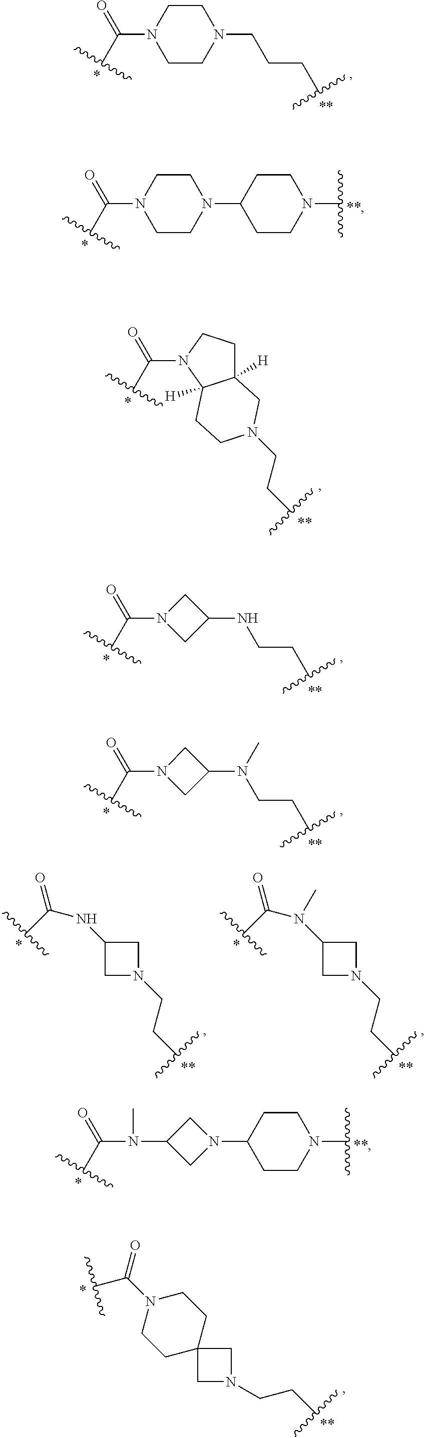

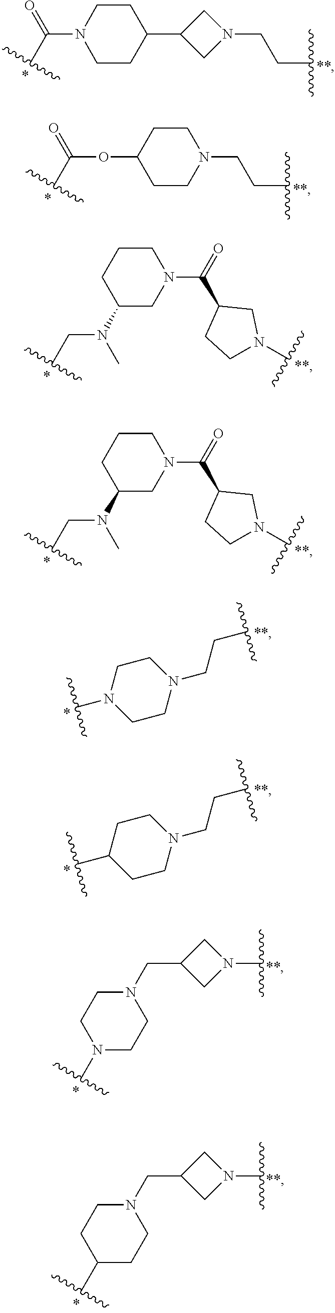

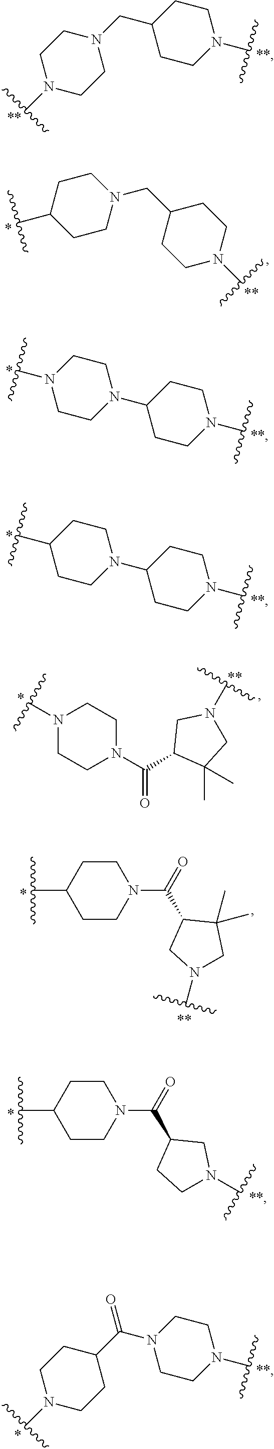

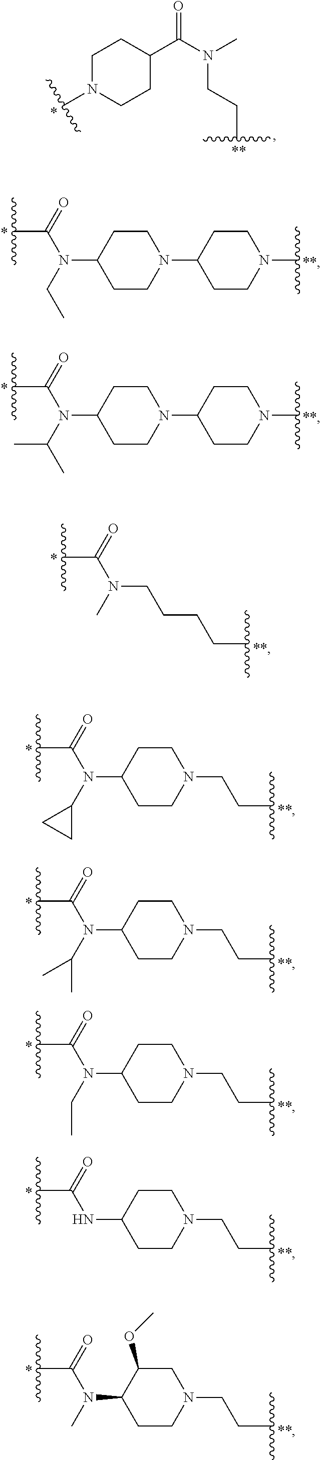

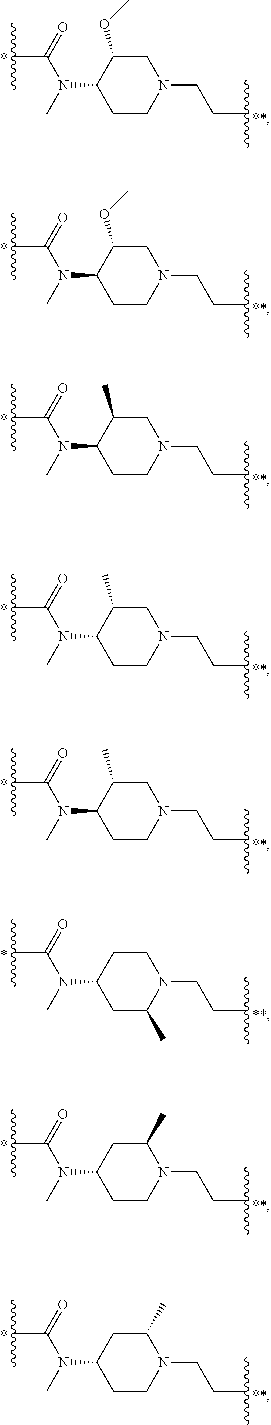

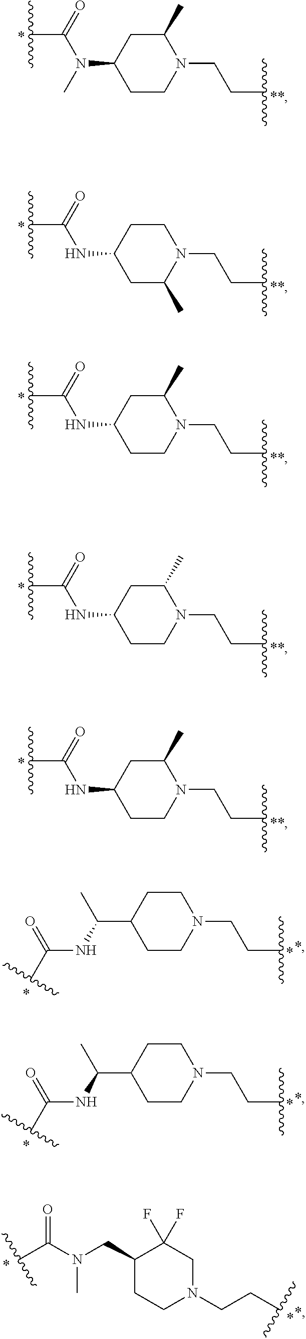

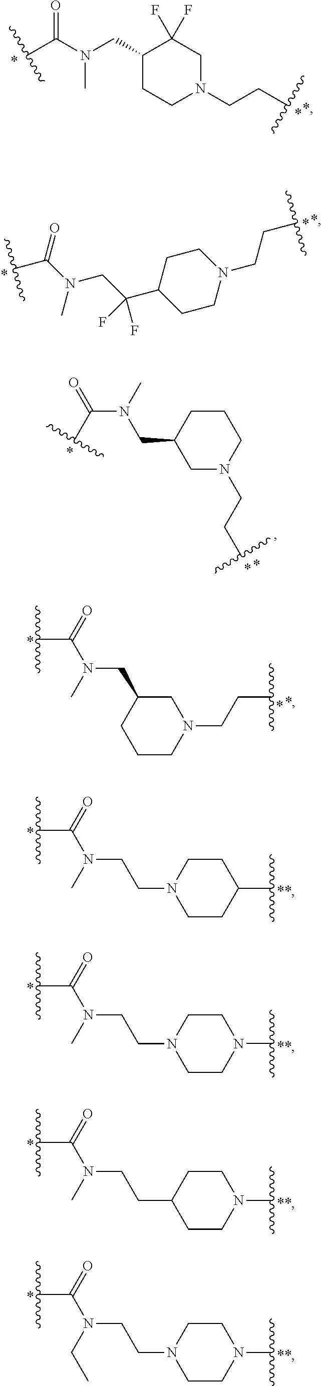

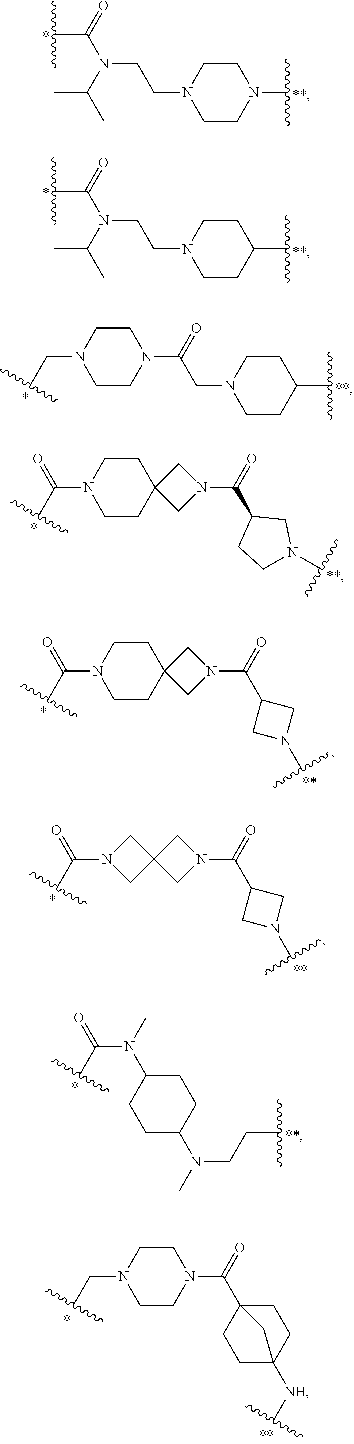

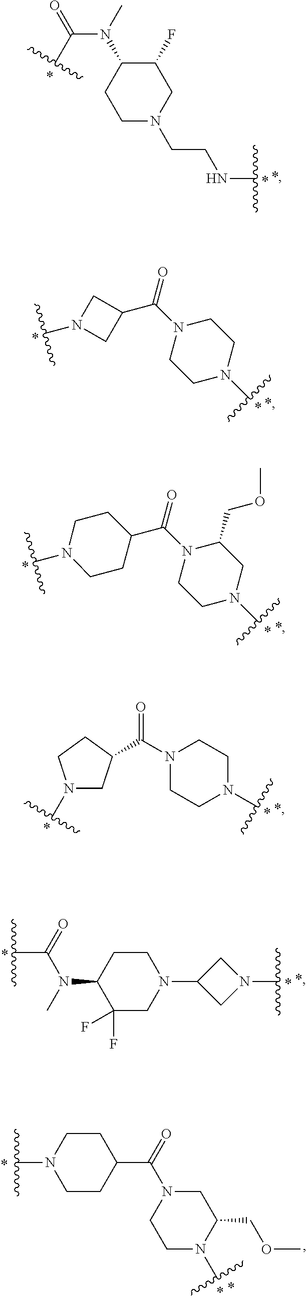 [SMILES: *NCCN1CC[C@H](N(C)C(*)=O)[C@H](F)C1 ; *N1CCN(C(=O)C2CN(*)C2)CC1 ; *N1CCC(C(=O)N2CCN(*)C[C@H]2COC)CC1 ; *N1CCN(C(=O)[C@H]2CCN(*)C2)CC1 ; *C(=O)N(C)[C@H]1CCN(C2CN(*)C2)CC1(F)F ; *N1CCC(C(=O)N2CCN(*)[C@@H](COC)C2)CC1]

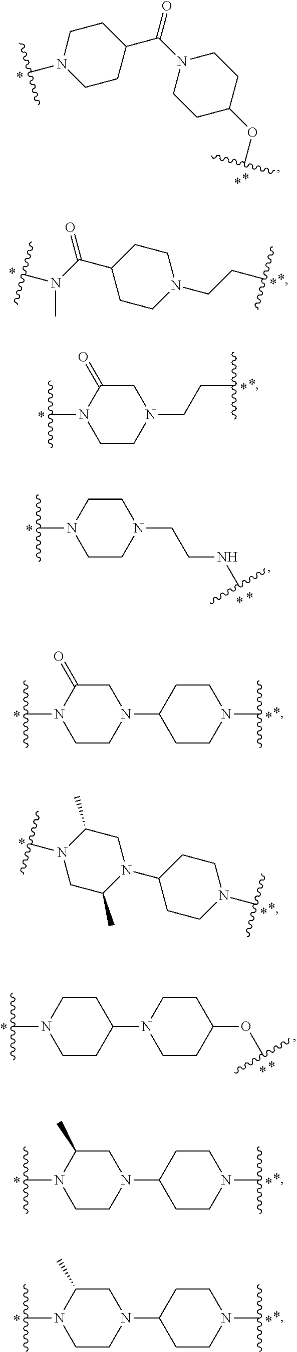

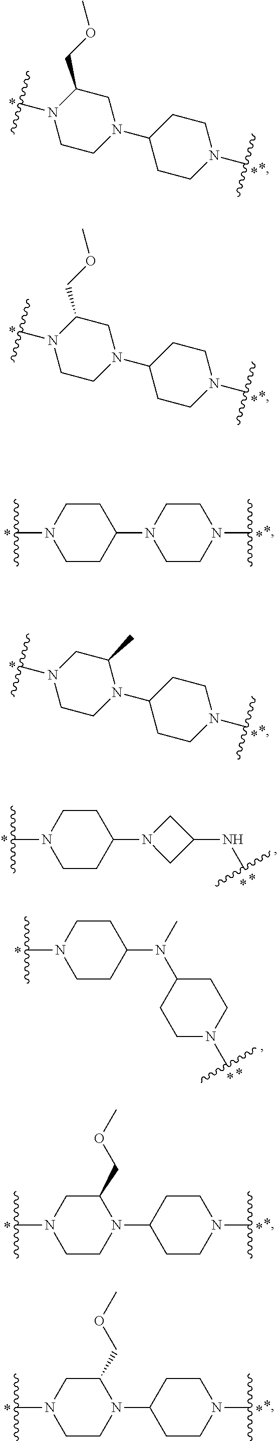

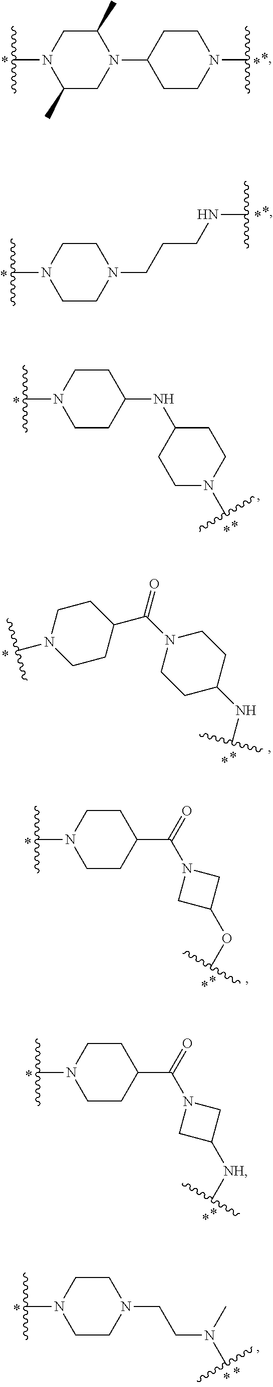

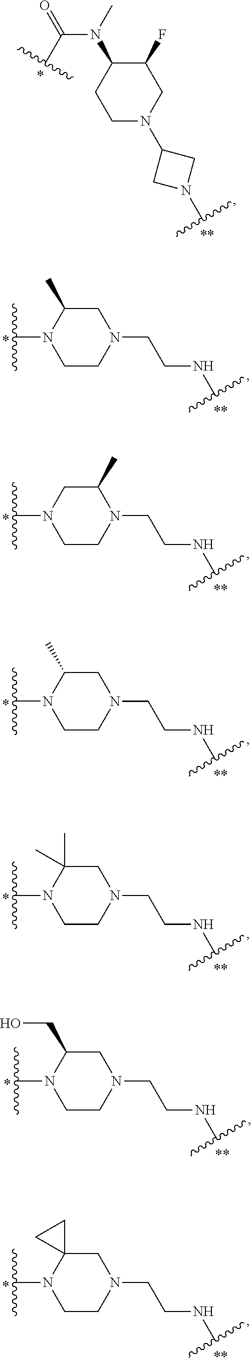

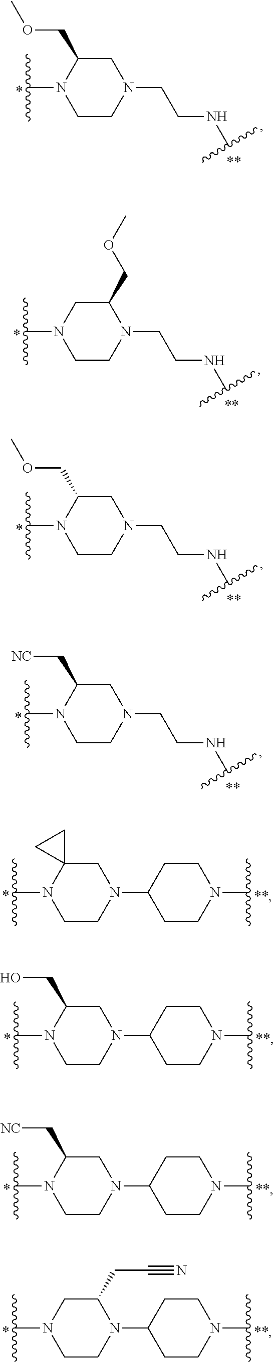

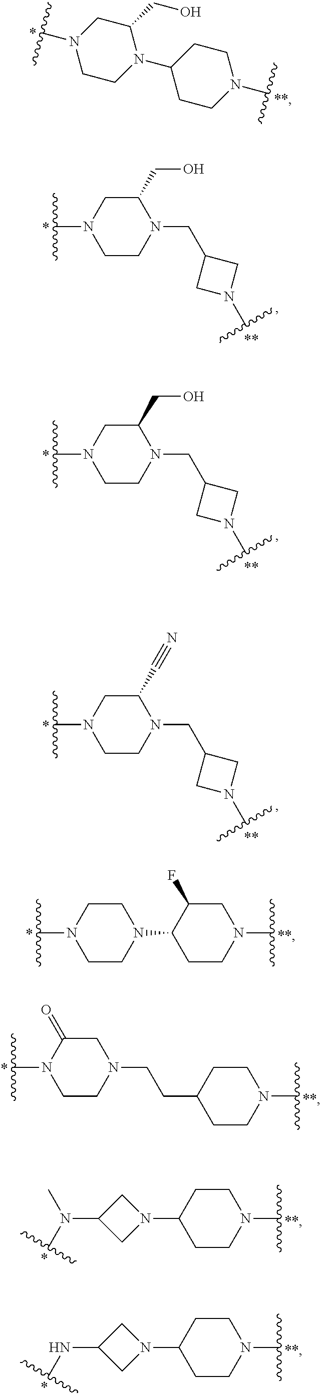

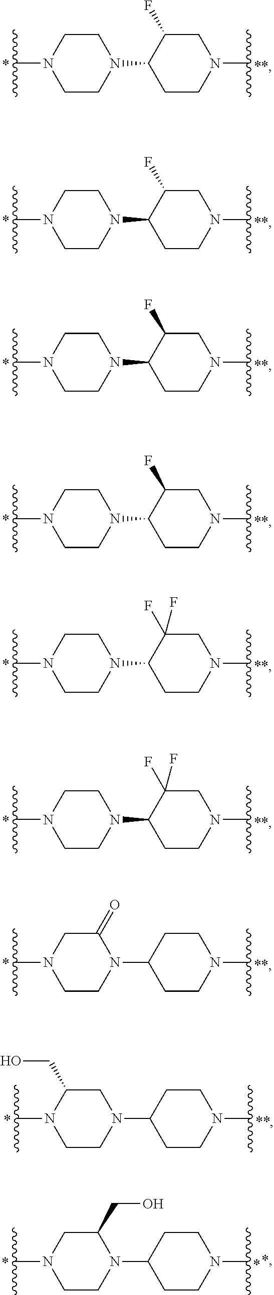

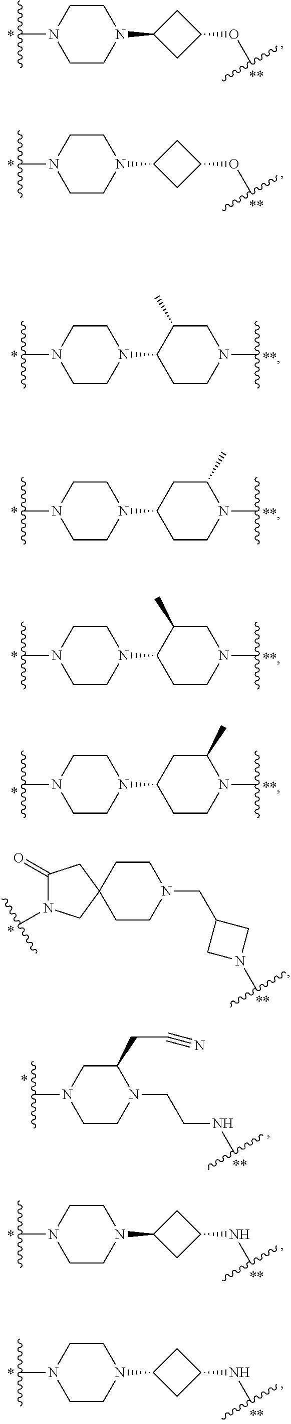

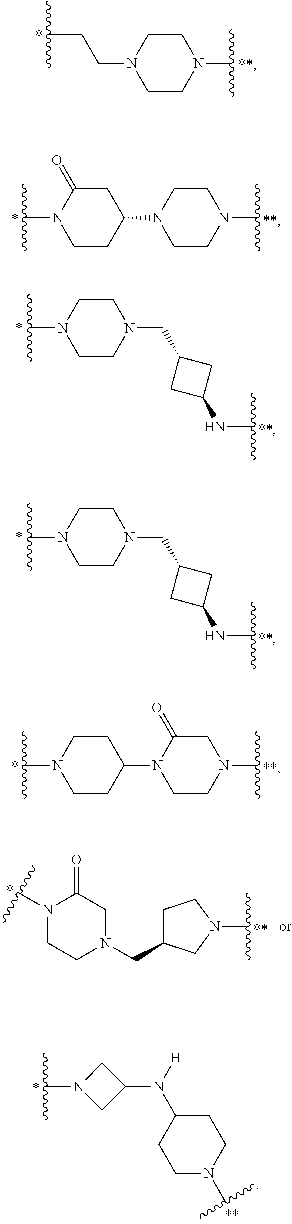

Aspect 27. The compound of any one of the preceding aspects, wherein L⁴ is independently selected from a single bond, —O—, —NR—, —(CR^{a}R^{b})ₙ₈—, —O(CR^{a}R^{b})ₙ₈—, —NR^{a}(CRₐR^{b})ₙ₈— or —C(O)—,
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 28. The compound of any one of the preceding aspects, wherein L⁴ is independently selected from a single bond.

Aspect 29. The compound of any one of the preceding aspects, wherein X⁷ is independently selected from —CR, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 30. The compound of any one of the preceding aspects, wherein X⁷ is independently selected from —CH, —C(CH₃), or N; preferably X⁷ is independently selected from —CH.

Aspect 31. The compound of any one of the preceding aspects, wherein X⁸ is independently selected from —NR^{a}—, —O—, —S— and —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy. —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 32. The compound of any one of the preceding aspects, wherein X⁵ is independently selected from —NH— and —CH₂—; preferably X⁸ is independently selected from —CH₂—.

Aspect 33. The compound of any one of the preceding aspects, wherein

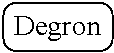

is selected from

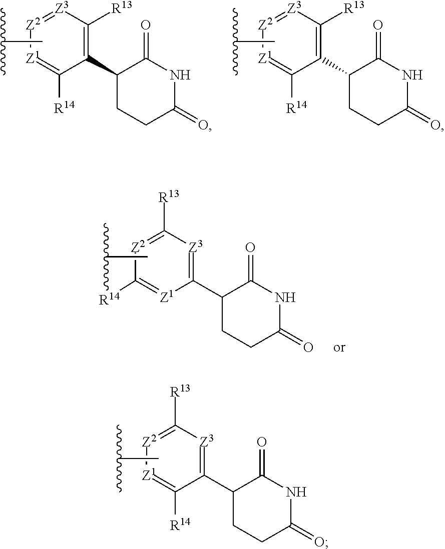

preferably,

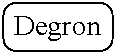

is selected from

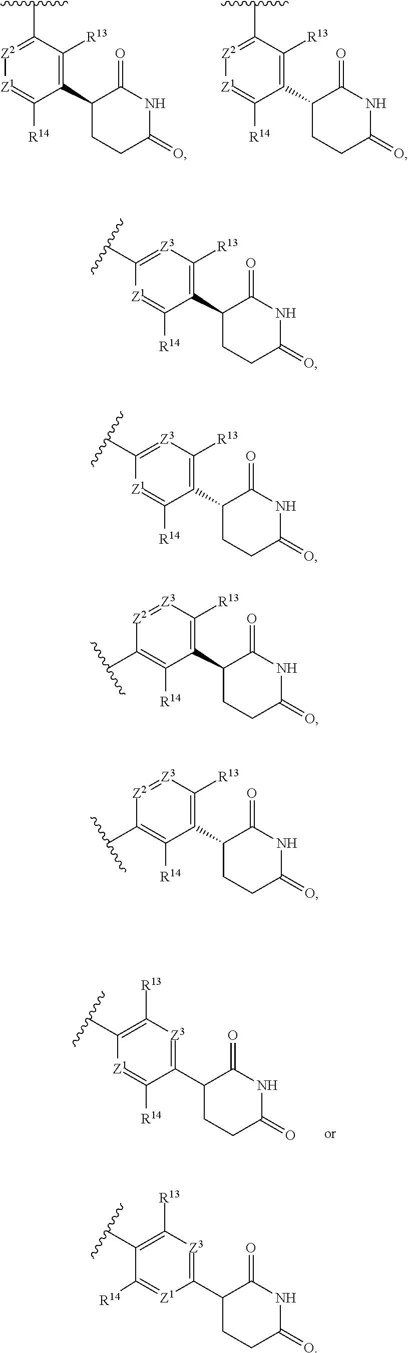

Aspect 34. The compound of any one of the preceding aspects, wherein at most one of Z¹, Z² and Z³ is N.

Aspect 35. The compound of any one of the preceding aspects, wherein Z¹, Z² and Z³ are each independently CR.

Aspect 36. The compound of any one of the preceding aspects, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Ze};

R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};

R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 37. The compound of any one of the preceding aspects, wherein R^{Z} is selected from H, —CH₃, —C₂H₅, F, —CH₂F, —CHF₂, —CF₃, —OCH₃, —OCH₂H₅, —C₃H₇, —OCH₂F, —OCHF₂, —OCH₂CF₃, —OCF₃, —SCF₃, —CF₃ or —CH(OH)CH₃.

Aspect 38. The compound of any one of the preceding aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}. —COR^{13a}, —CO₂R^{13a}, —CONR^{13a}R^{13b}, —NR^{13a}R^{13b}, —NR^{13a}COR^{13b}, —NR^{13c}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}R^{13d}, COR^{13d}, —NR^{13c}CO₂R^{13d}, or —NR^{13c}SO₂R^{13d}.

R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

Aspect 39. The compound of any one of the preceding aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

Aspect 40. The compound of any one of the preceding aspects, wherein

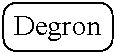

is

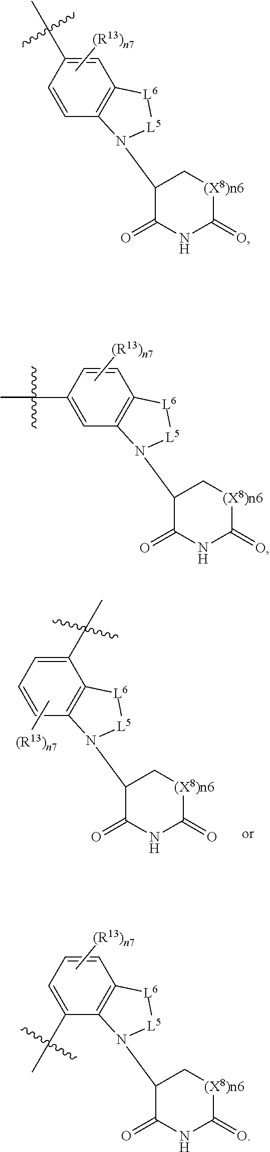

Aspect 41. The compound of any one of the preceding aspects, wherein L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 42. The compound of any one of the preceding aspects, wherein L¹ and L⁶ are each independently a single bond,

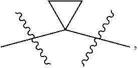 [SMILES: *C1(*)CC1]

—O—, —NH—, —NMe-, —N(CH₂CH₃)—, —CH₂—, —CHF—, —CF₂—, —C(CH₃)₂— or —CO— (preferably L⁵ is —CO— or —CH₂—, and L⁶ is,

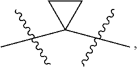 [SMILES: *C1(*)CC1]

—O—, —NH—, —NMe-, —N(CH₂CH₃)—, —CH₂—, —CHF—, —CF₂—, —C(CH₃)₂— or —CO—);
X⁸ is CH₂; and
n6 is 0 or 1.

Aspect 43. The compound of any one of the preceding aspects, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13a}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}—, —SO₂R^{13c}, —SO₂NR^{13c}R^{13d}, —COR^{13c}, —CO₂R^{13c}, —CONR^{13c}R^{13d}, —NR^{13c}OR^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

Aspect 44. The compound of any one of the preceding aspects, wherein R¹³ is independently selected from hydrogen, F, Cl, Br, I, CN, —C₁-C₈alkyl, or —C₁-C₈alkoxy; preferably R¹³ is independently selected from hydrogen, F, Cl, Br, I, CN, -Me, -Et, —C₃H₇, —C₄H₉, —OMe, -OEt, —OC₃H₇ or —OC₄H₉;
n₇ is 0, 1 or 2.

Aspect 45. The compound of any one of the preceding aspects, wherein

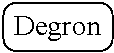

is

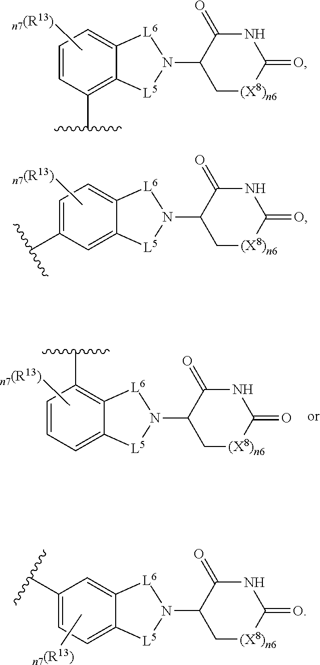

Aspect 46. The compound of any one of the preceding aspects, wherein L¹ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 47. The compound of any one of the preceding aspects, wherein L¹ and L⁶ are each independently a single bond,

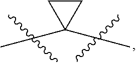 [SMILES: *C1(*)CC1]

—O—, —NH—, —NMe-, —N(CH₂CH₃)—, —CH₂—, —CHF—, —CF₂—, —C(CH₃)₂— or —CO— (preferably L¹ and L⁶ are each independently —CO— or —CH₂—);
X is CH₂; and
n6 is 0 or 1.

Aspect 48. The compound of any one of the preceding aspects, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}; —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR¹³OR^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d}.
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5-to 12-membered heteroaryl.

Aspect 49. The compound of any one of the preceding aspects, wherein R¹³ is independently selected from hydrogen, F, Cl, Br, I, CN, —C₁-C₈alkyl, or —C₁-C₈alkoxy; preferably R¹³ is independently selected from hydrogen, F, Cl, Br, I, CN, -Me, -Et, —C₃H₇, —C₄H₉, —OMe, -OEt, —OC₃H₇ or —OC₄H₉;
n7 is 0, 1 or 2.

Aspect 50. The compound of any one of the preceding aspects, wherein

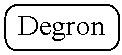

is

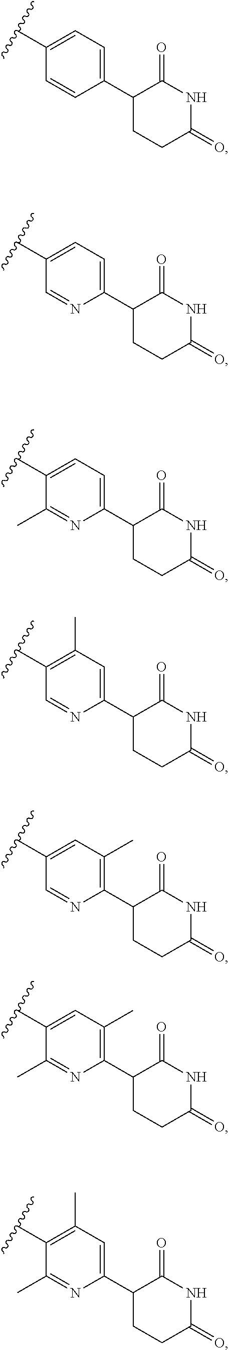

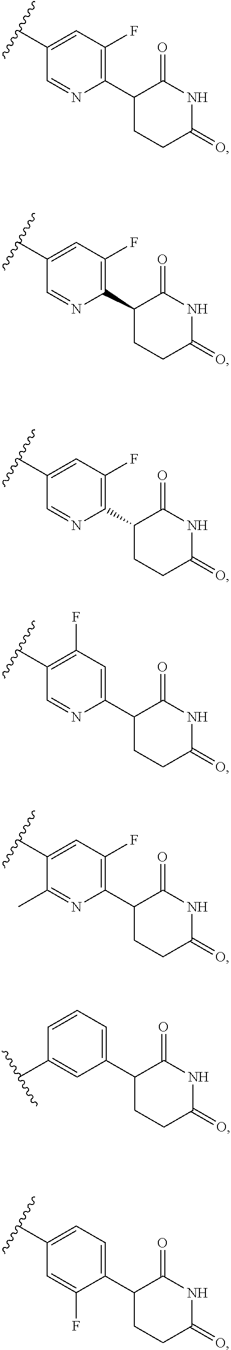

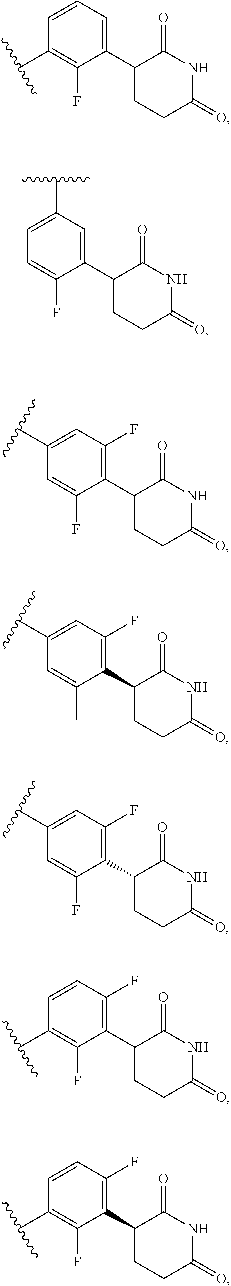

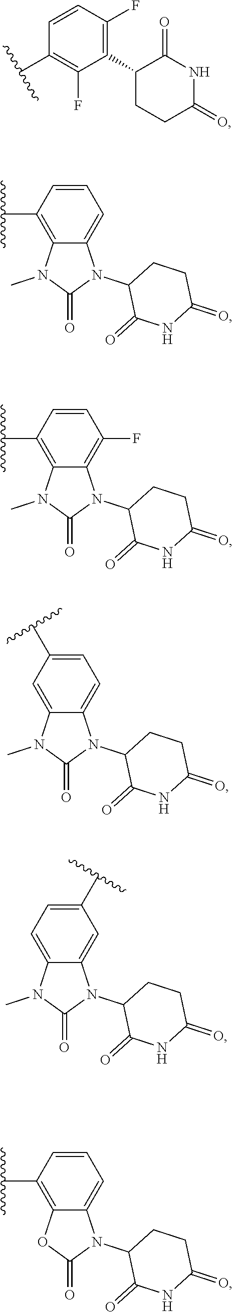 [SMILES: *c1ccc(F)c([C@H]2CCC(=O)NC2=O)c1F ; *c1cccc2c1n(C)c(=O)n2C1CCC(=O)NC1=O ; *c1ccc(F)c2c1n(C)c(=O)n2C1CCC(=O)NC1=O ; *c1ccc2c(c1)n(C)c(=O)n2C1CCC(=O)NC1=O ; *c1ccc2c(c1)n(C1CCC(=O)NC1=O)c(=O)n2C ; *c1cccc2c1oc(=O)n2C1CCC(=O)NC1=O]

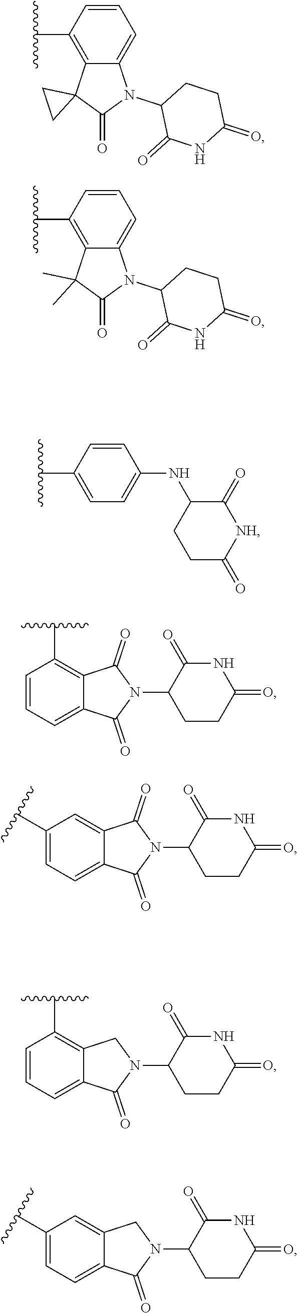

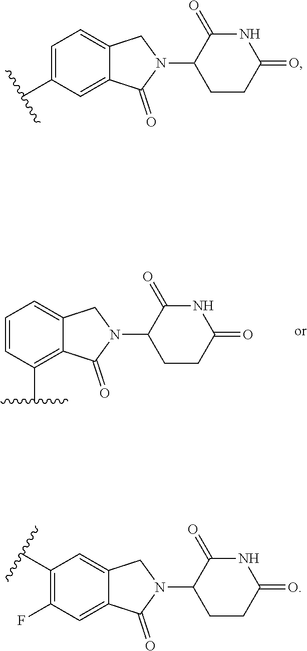 [SMILES: *c1ccc2c(c1)C(=O)N(C1CCC(=O)NC1=O)C2 ; *c1cccc2c1C(=O)N(C1CCC(=O)NC1=O)C2 ; *c1cc2c(cc1F)C(=O)N(C1CCC(=O)NC1=O)C2]

Aspect 51. The compound of any one of the preceding aspects, wherein the

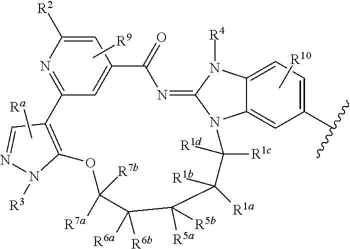

moiety is

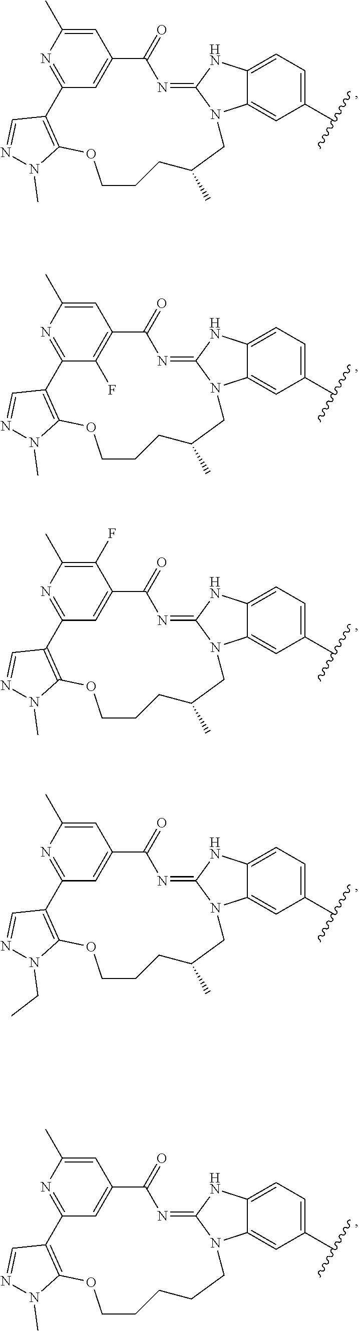 [SMILES: *c1ccc2c(c1)N1C[C@H](C)CCCOc3c(cnn3C)-c3cc(cc(C)n3)C(=O)/N=C/1N2 ; *c1ccc2c(c1)N1C[C@H](C)CCCOc3c(cnn3C)-c3nc(C)cc(c3F)C(=O)/N=C/1N2 ; *c1ccc2c(c1)N1C[C@H](C)CCCOc3c(cnn3C)-c3cc(c(F)c(C)n3)C(=O)/N=C/1N2 ; *c1ccc2c(c1)N1C[C@H](C)CCCOc3c(cnn3CC)-c3cc(cc(C)n3)C(=O)/N=C/1N2 ; *c1ccc2c(c1)N1CCCCCOc3c(cnn3C)-c3cc(cc(C)n3)C(=O)/N=C/1N2]

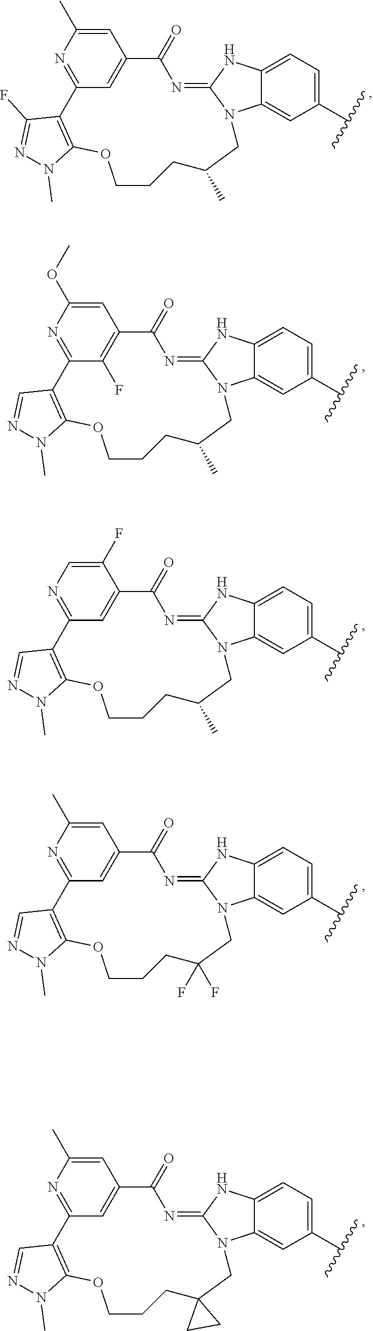

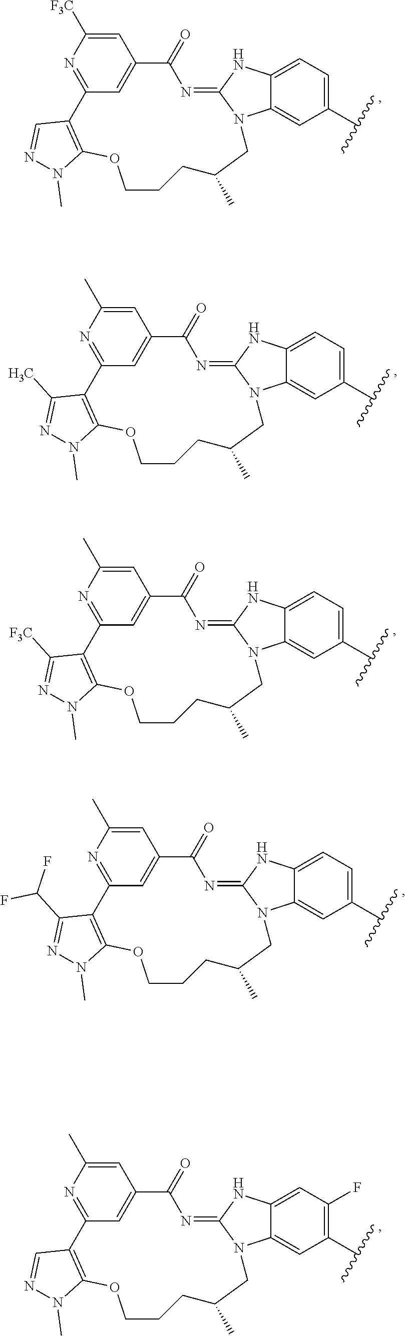 [SMILES: *c1ccc2c(c1)N1C[C@H](C)CCCOc3c(cnn3C)-c3cc(cc(C(F)(F)F)n3)C(=O)/N=C/1N2 ; *c1ccc2c(c1)N1C[C@H](C)CCCOc3c(c(C)nn3C)-c3cc(cc(C)n3)C(=O)/N=C/1N2 ; *c1ccc2c(c1)N1C[C@H](C)CCCOc3c(c(C(F)(F)F)nn3C)-c3cc(cc(C)n3)C(=O)/N=C/1N2 ; *c1ccc2c(c1)N1C[C@H](C)CCCOc3c(c(C(F)F)nn3C)-c3cc(cc(C)n3)C(=O)/N=C/1N2 ; *c1cc2c(cc1F)N/C1=N\C(=O)c3cc(C)nc(c3)-c3cnn(C)c3OCCC[C@@H](C)CN12]

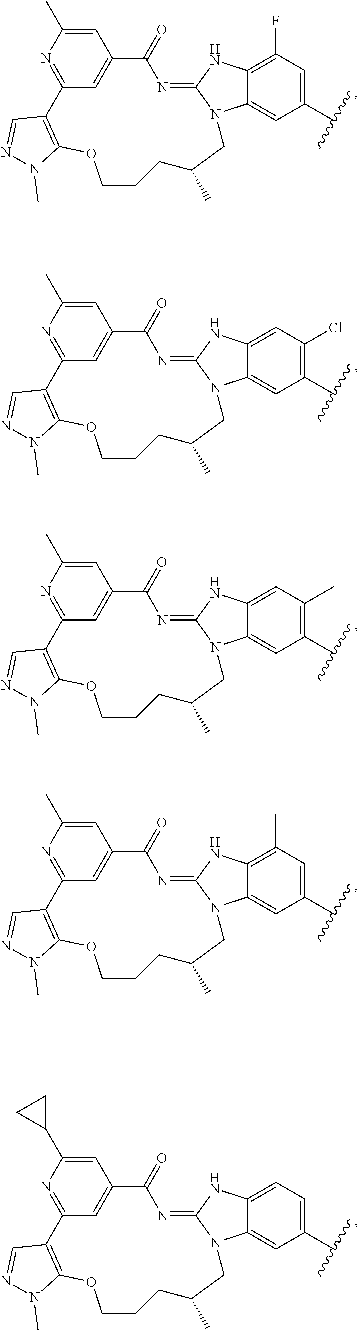 [SMILES: *c1cc(F)c2c(c1)N1C[C@H](C)CCCOc3c(cnn3C)-c3cc(cc(C)n3)C(=O)/N=C/1N2 ; *c1cc2c(cc1Cl)N/C1=N\C(=O)c3cc(C)nc(c3)-c3cnn(C)c3OCCC[C@@H](C)CN12 ; *c1cc2c(cc1C)N/C1=N\C(=O)c3cc(C)nc(c3)-c3cnn(C)c3OCCC[C@@H](C)CN12 ; *c1cc(C)c2c(c1)N1C[C@H](C)CCCOc3c(cnn3C)-c3cc(cc(C)n3)C(=O)/N=C/1N2 ; *c1ccc2c(c1)N1C[C@H](C)CCCOc3c(cnn3C)-c3cc(cc(C4CC4)n3)C(=O)/N=C/1N2]

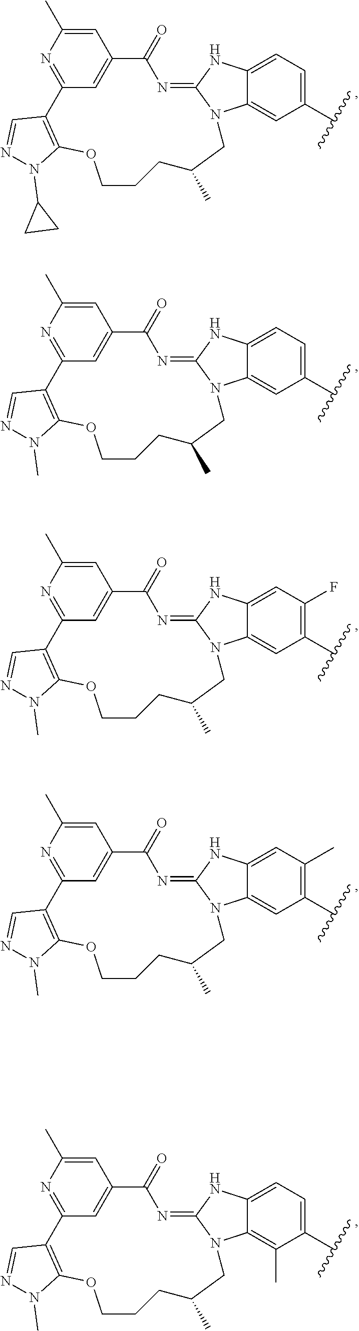

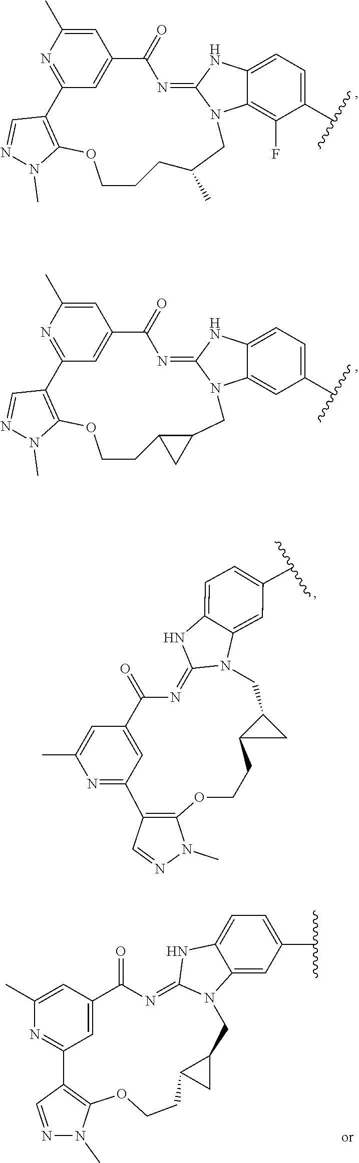 [SMILES: *c1ccc2c(c1F)N1C[C@H](C)CCCOc3c(cnn3C)-c3cc(cc(C)n3)C(=O)/N=C/1N2 ; *c1ccc2c(c1)N1CC3CC3CCOc3c(cnn3C)-c3cc(cc(C)n3)C(=O)/N=C/1N2 ; *c1ccc2c(c1)N1C[C@@H]3C[C@H]3CCOc3c(cnn3C)-c3cc(cc(C)n3)C(=O)/N=C/1N2 ; *c1ccc2c(c1)N1C[C@H]3C[C@@H]3CCOc3c(cnn3C)-c3cc(cc(C)n3)C(=O)/N=C/1N2]

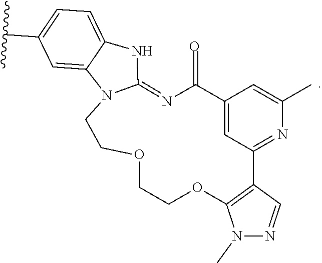

Aspect 52. The compound of any one of the preceding aspects is selected from

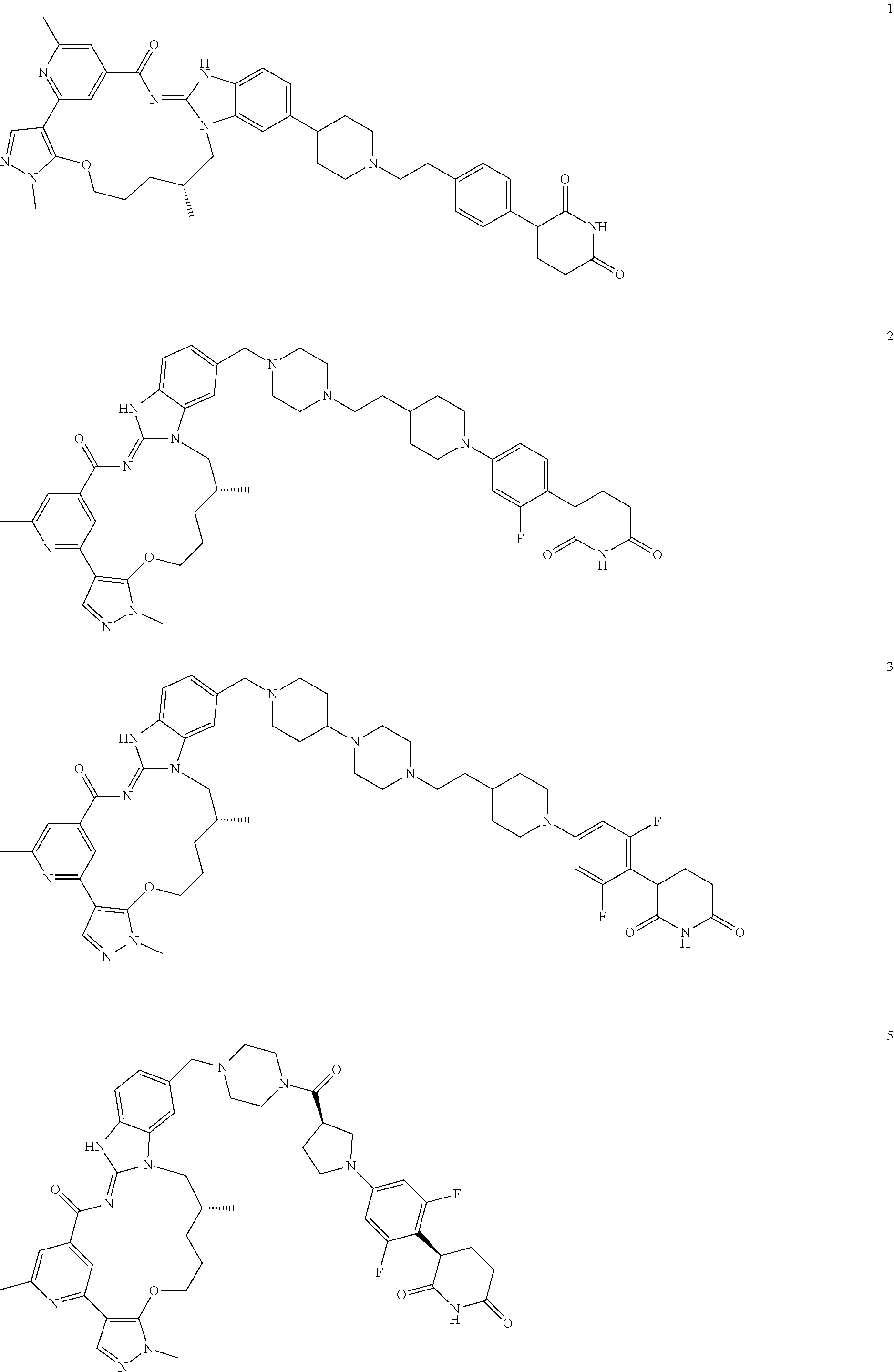 [SMILES: Cc1cc2cc(n1)-c1cnn(C)c1OCCC[C@@H](C)CN1/C(=N/C2=O)Nc2ccc(C3CCN(CCc4ccc(C5CCC(=O)NC5=O)cc4)CC3)cc21 ; Cc1cc2cc(n1)-c1cnn(C)c1OCCC[C@@H](C)CN1/C(=N/C2=O)Nc2ccc(CN3CCN(CCC4CCN(c5ccc(C6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(CN3CCC(N4CCN(CCC5CCN(c6cc(F)c(C7CCC(=O)NC7=O)c(F)c6)CC5)CC4)CC3)cc21 ; Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21]

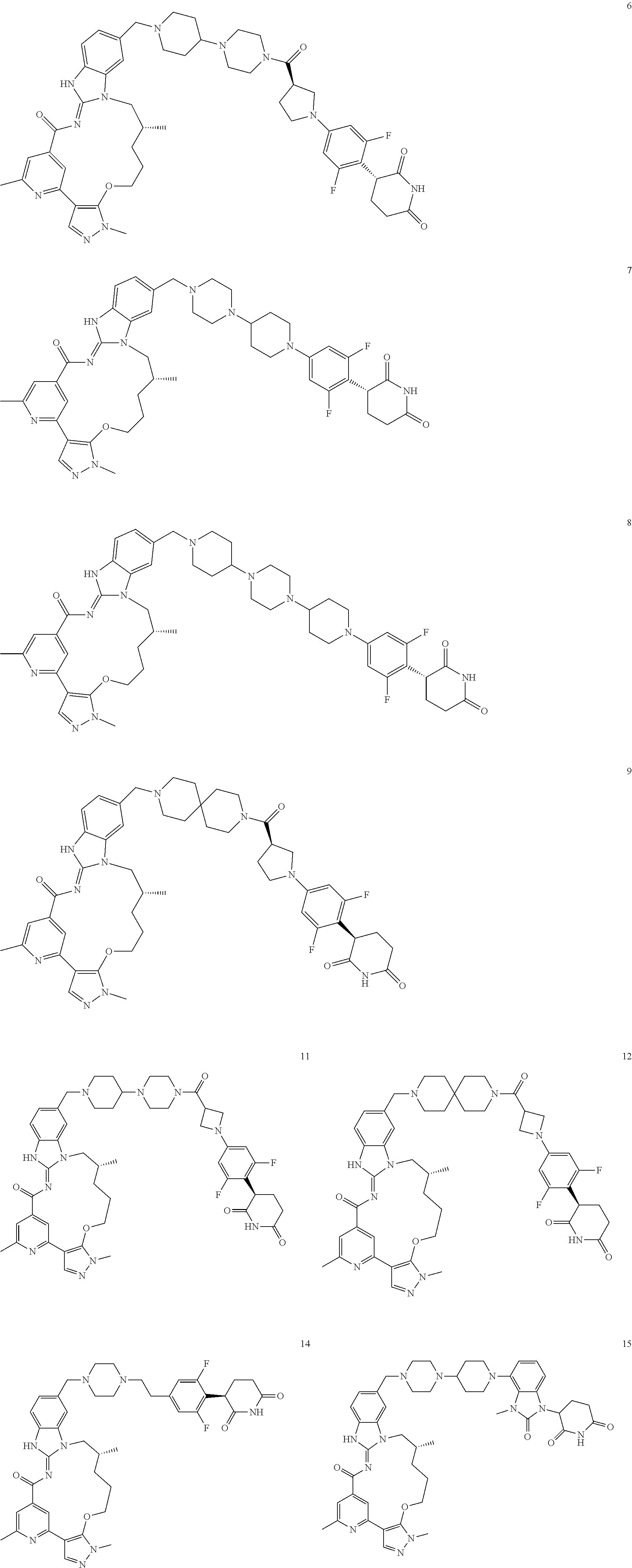

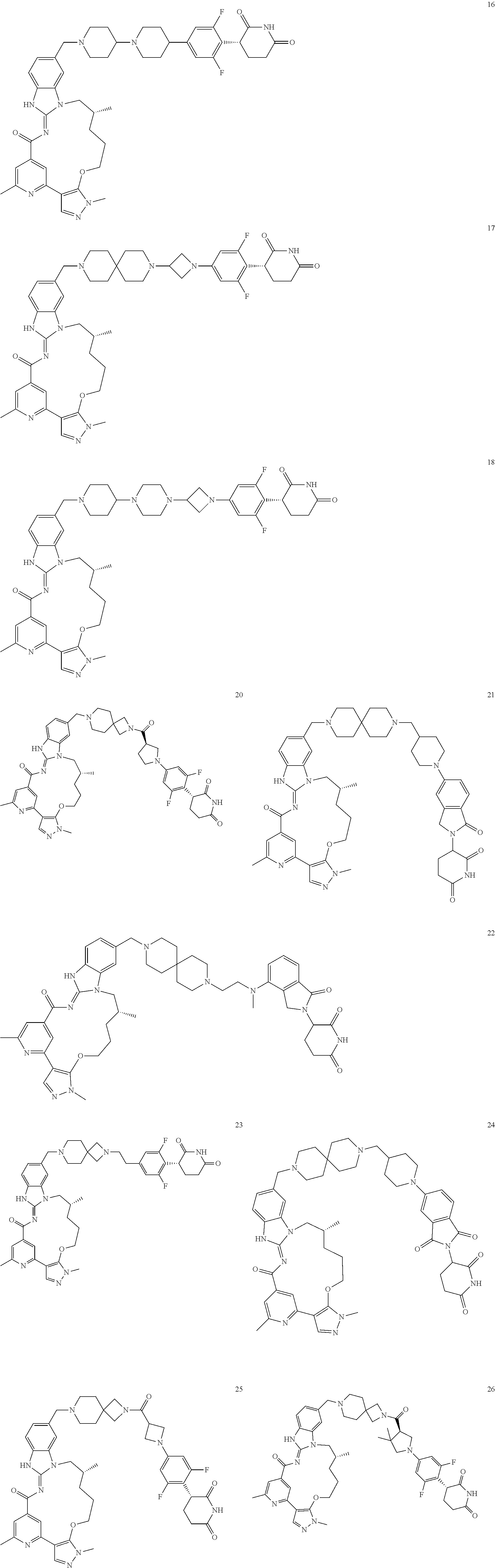

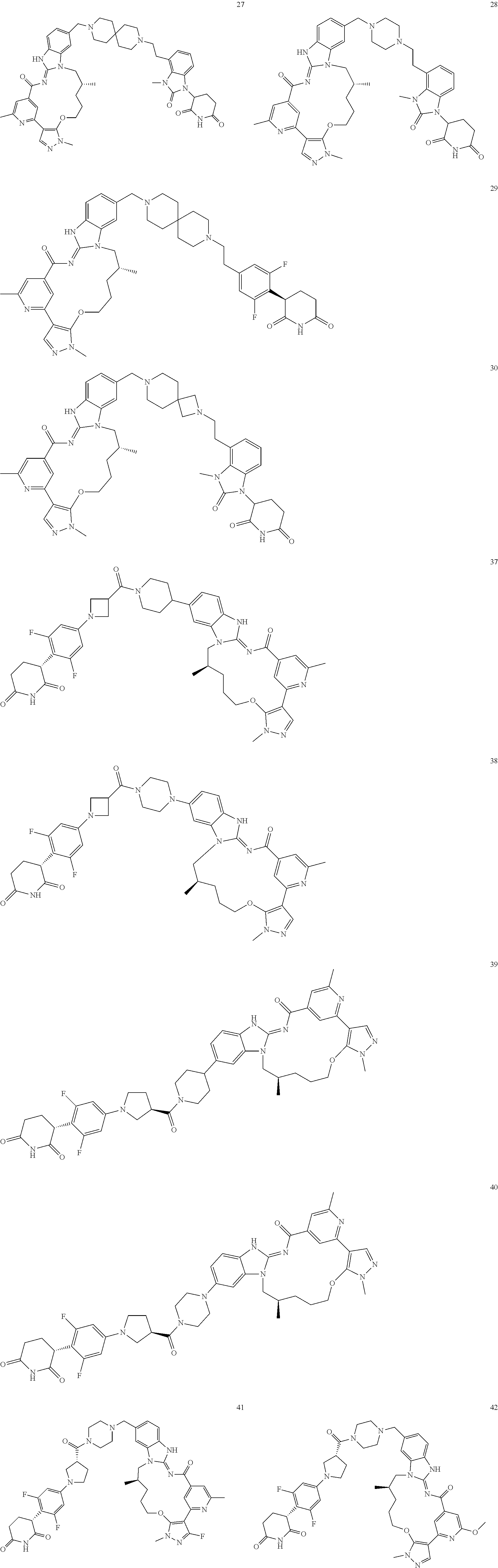

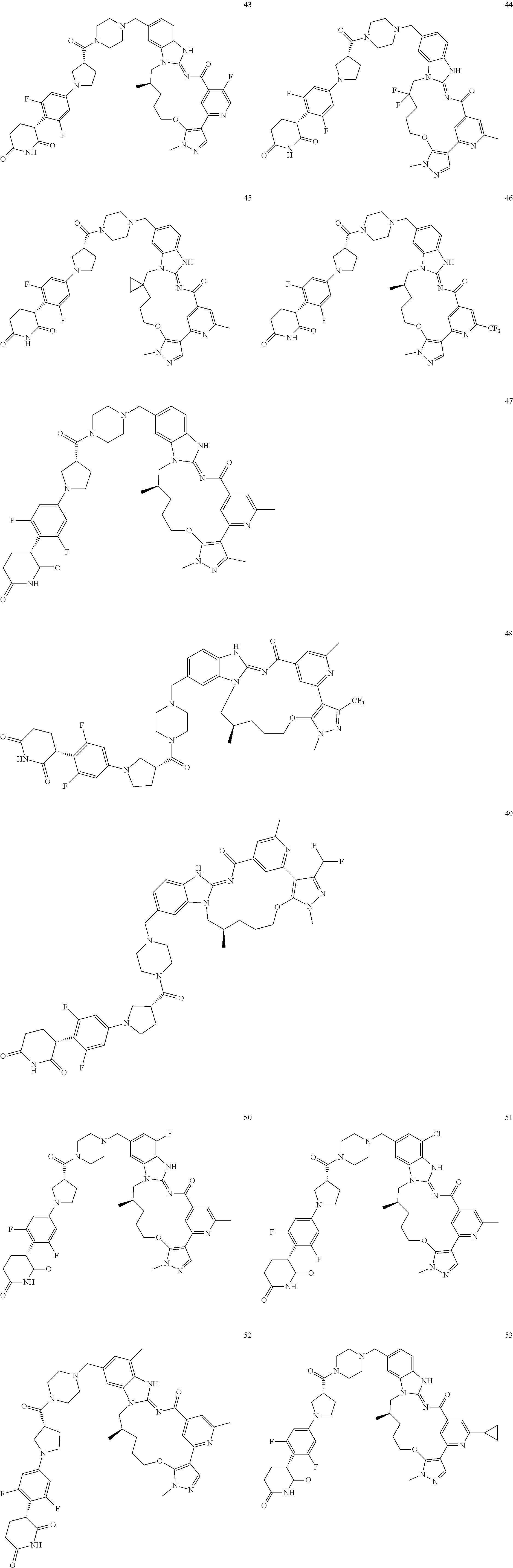

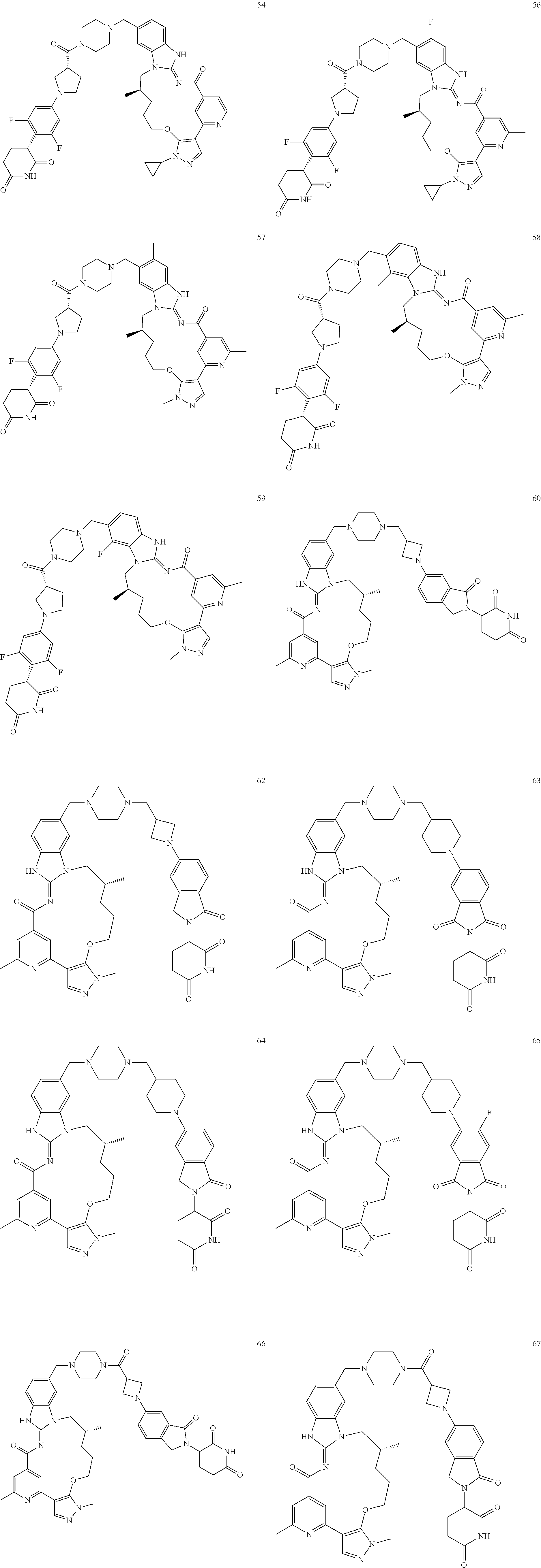

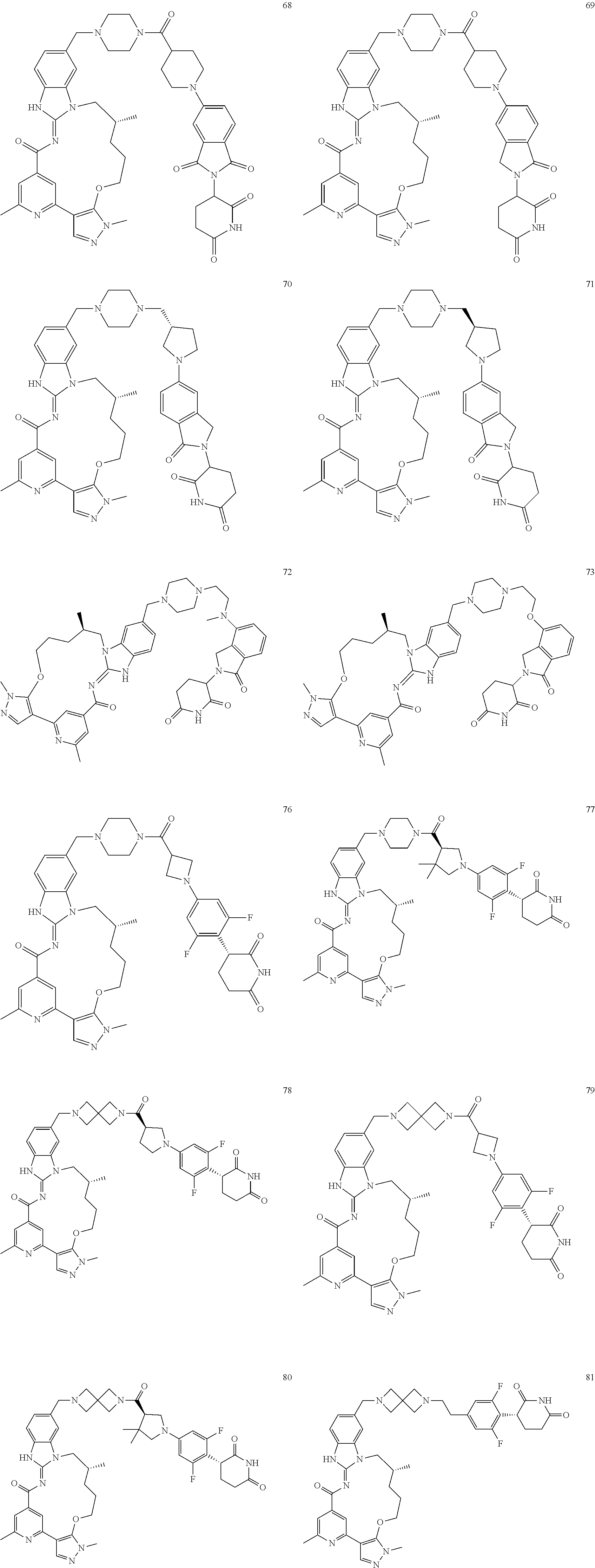

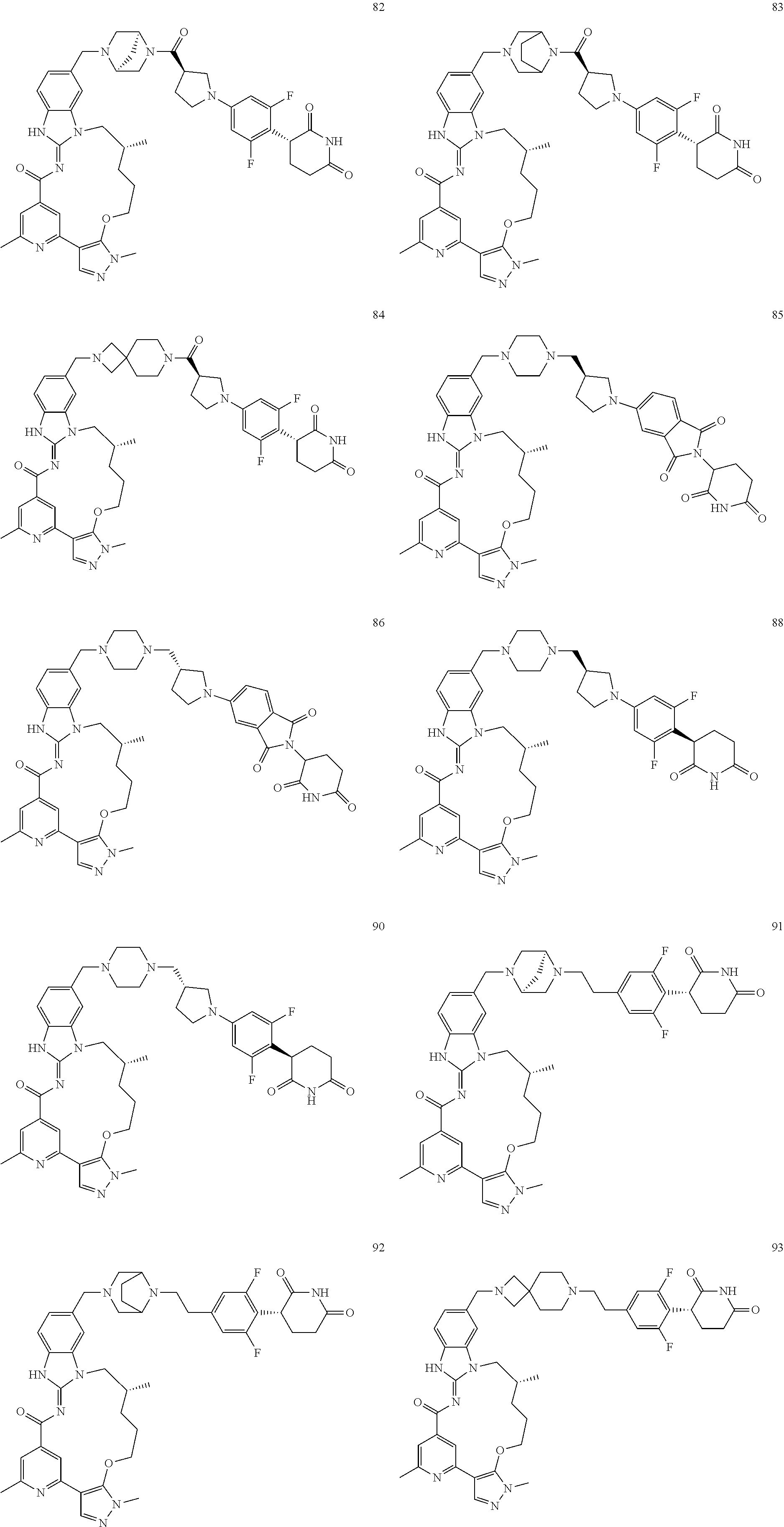

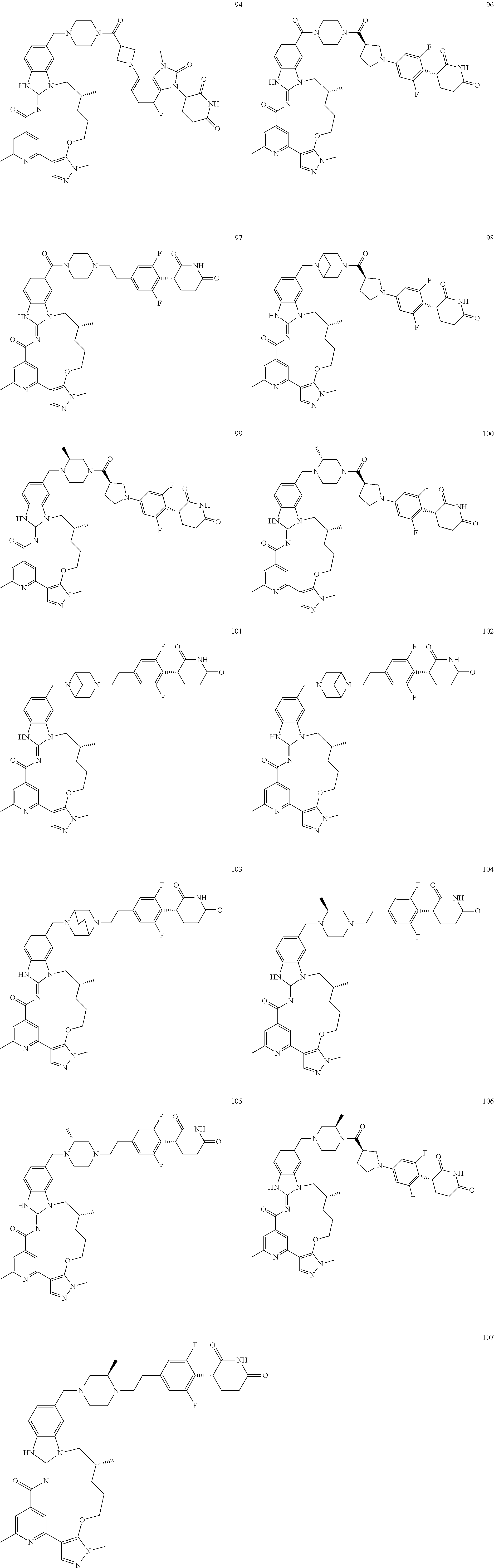

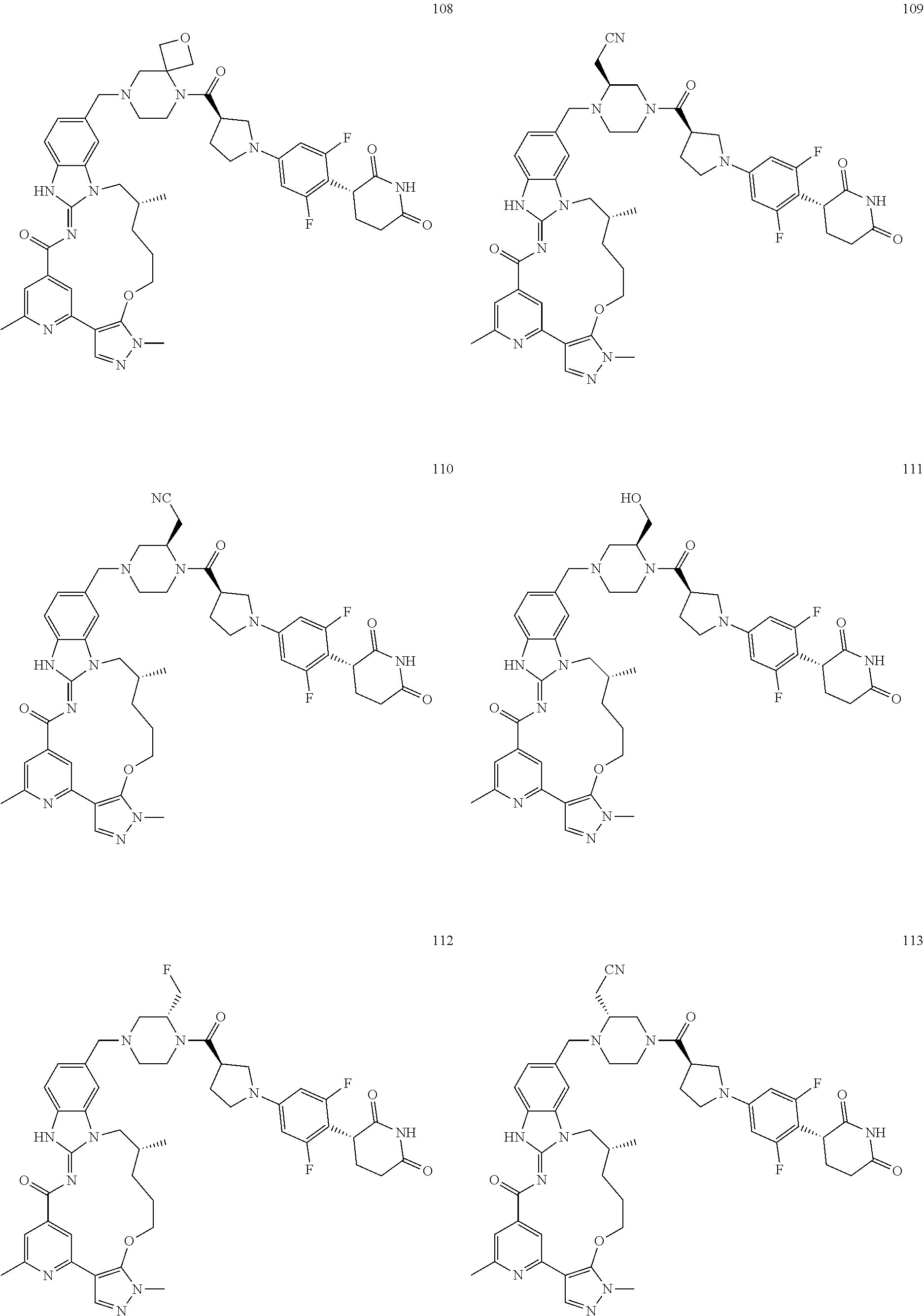

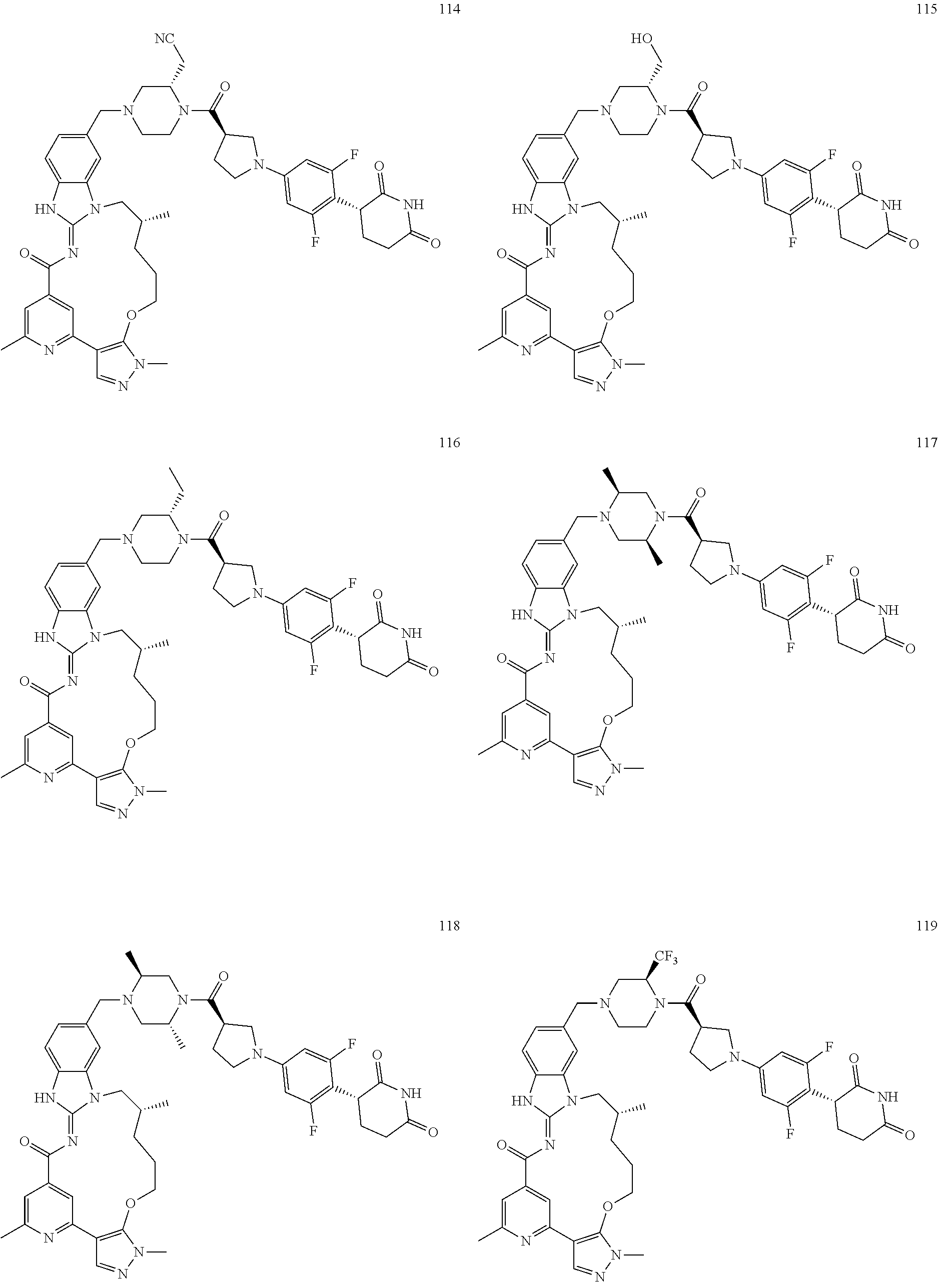

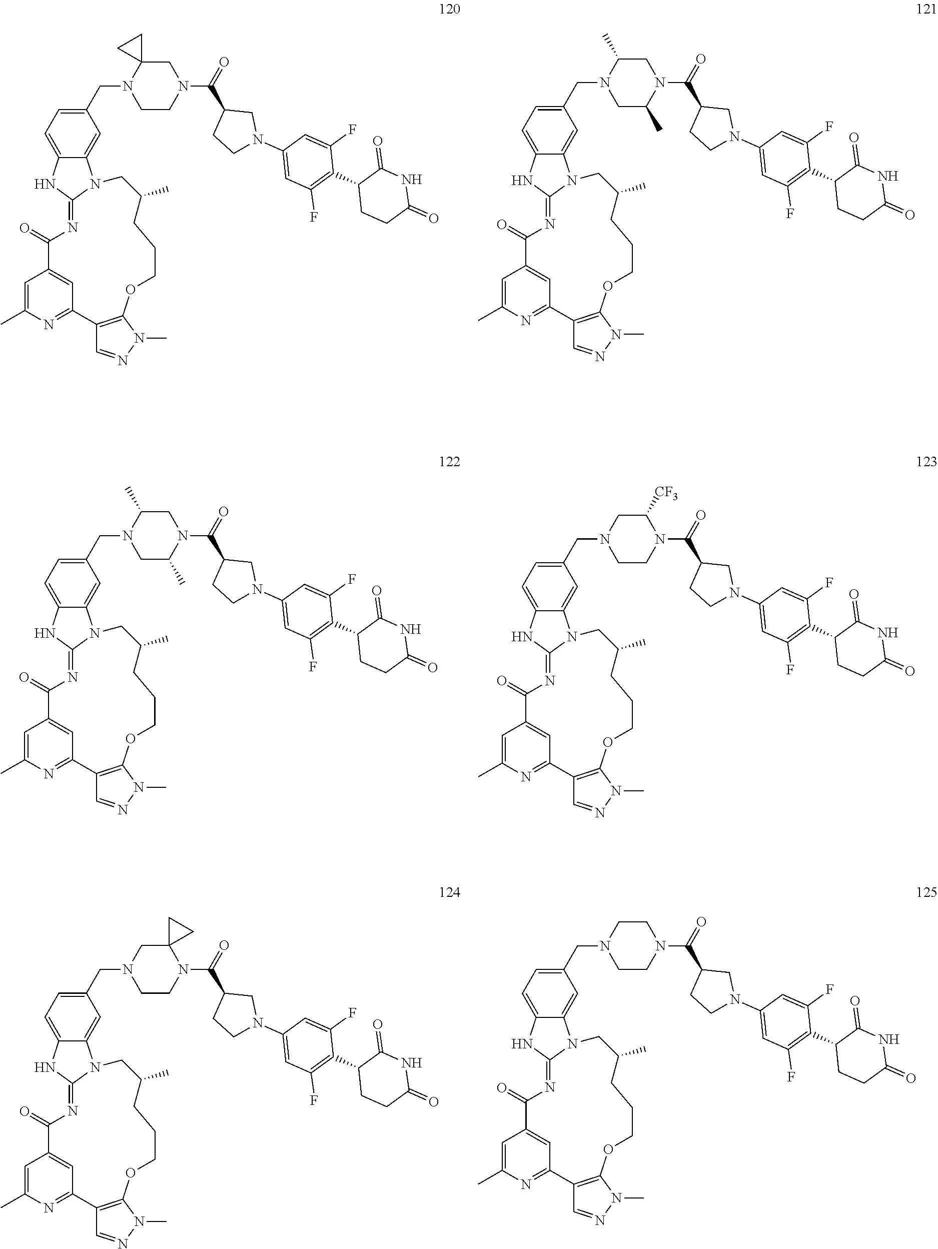

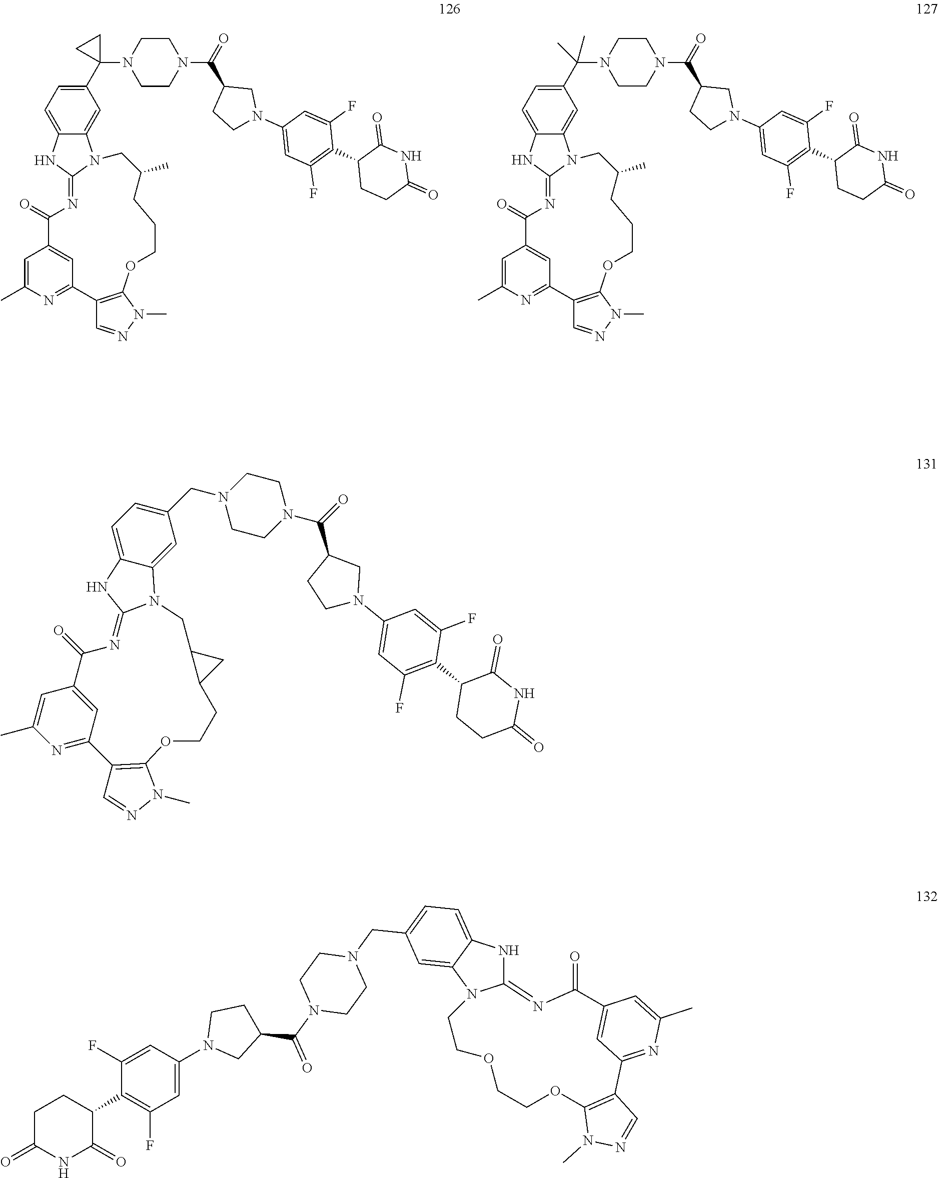

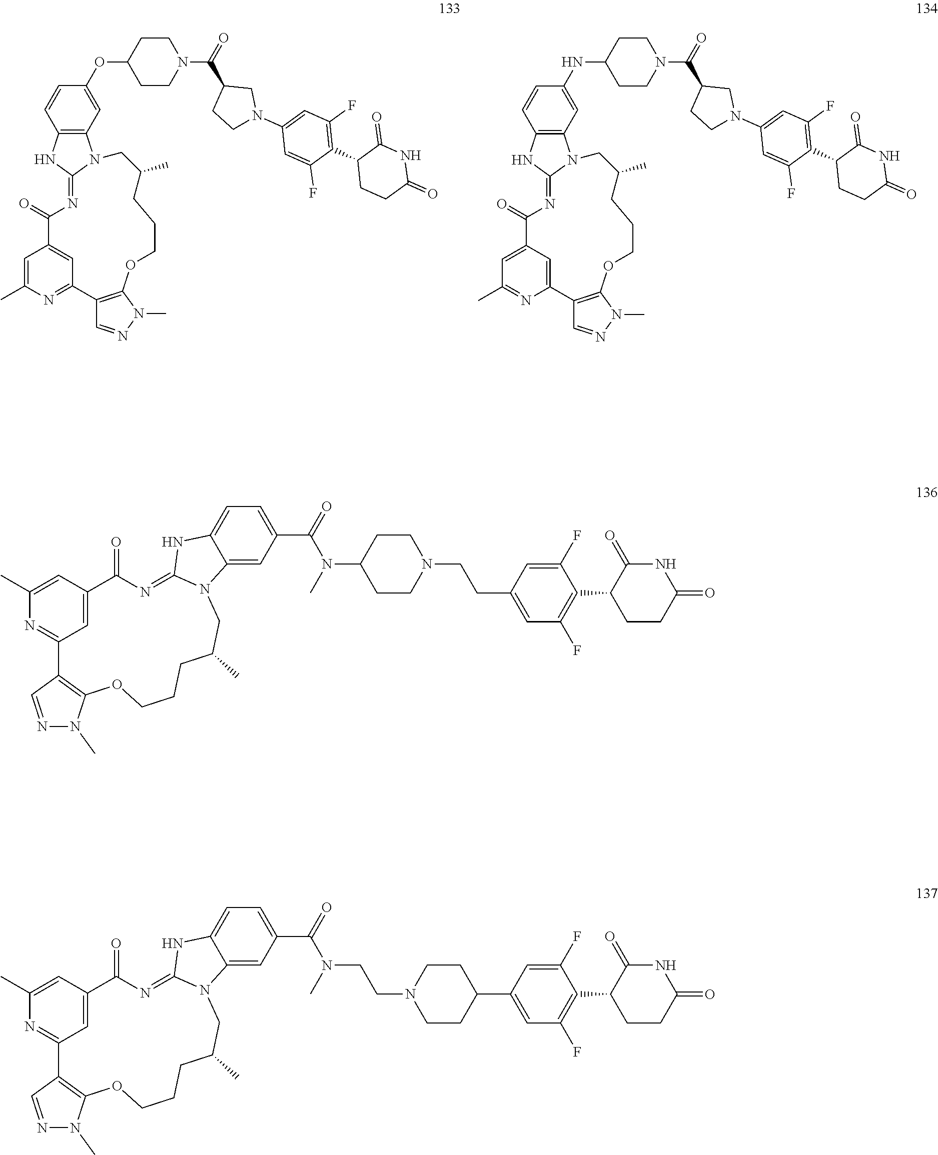 [SMILES: Cc1cc2cc(n1)-c1cnn(C)c1OCCC[C@@H](C)CN1/C(=N/C2=O)Nc2ccc(OC3CCN(C(=O)[C@@H]4CCN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(NC3CCN(C(=O)[C@@H]4CCN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C(=O)N(C)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)CCN3CCC(c4cc(F)c([C@H]5CCC(=O)NC5=O)c(F)c4)CC3)cc21]

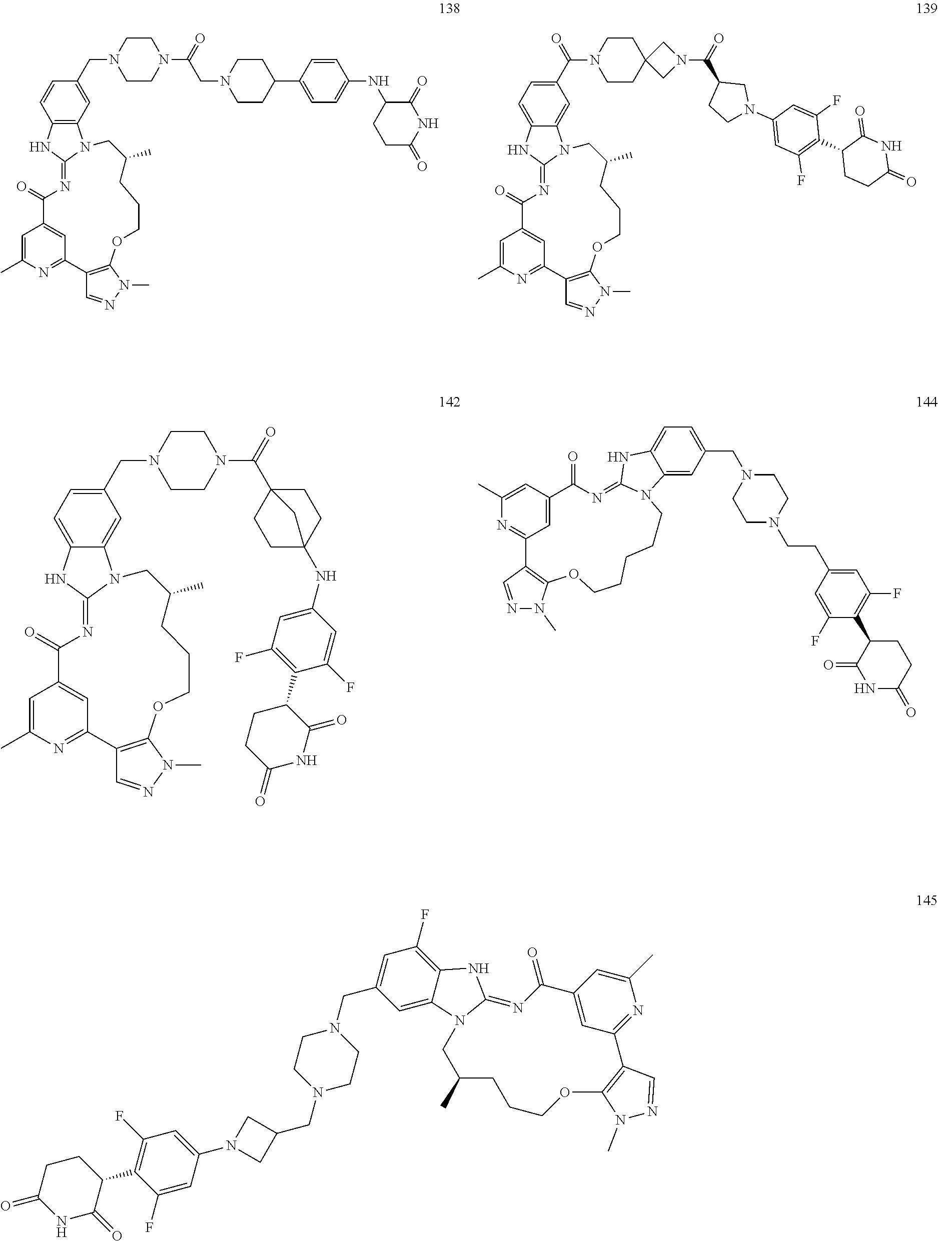

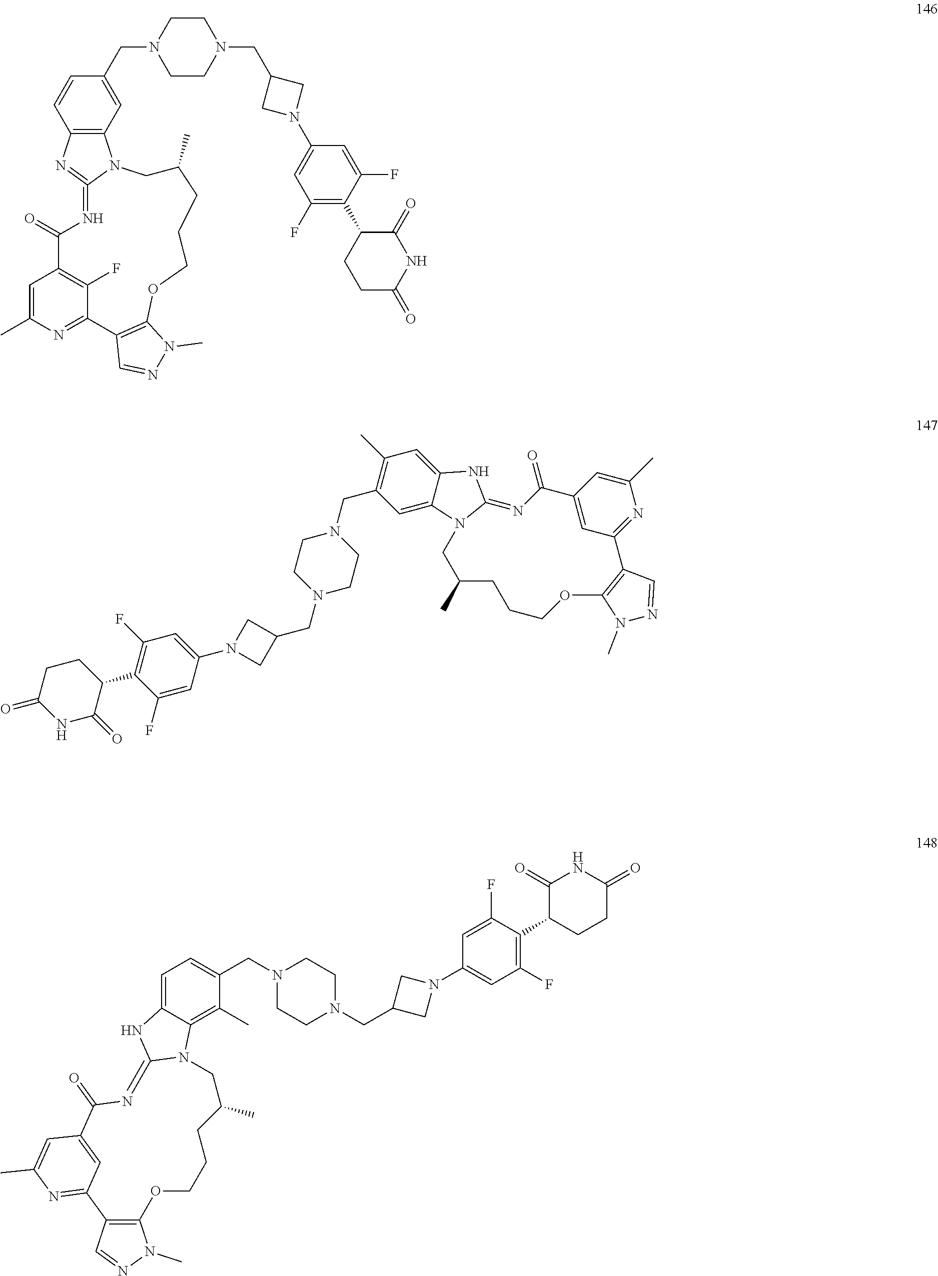

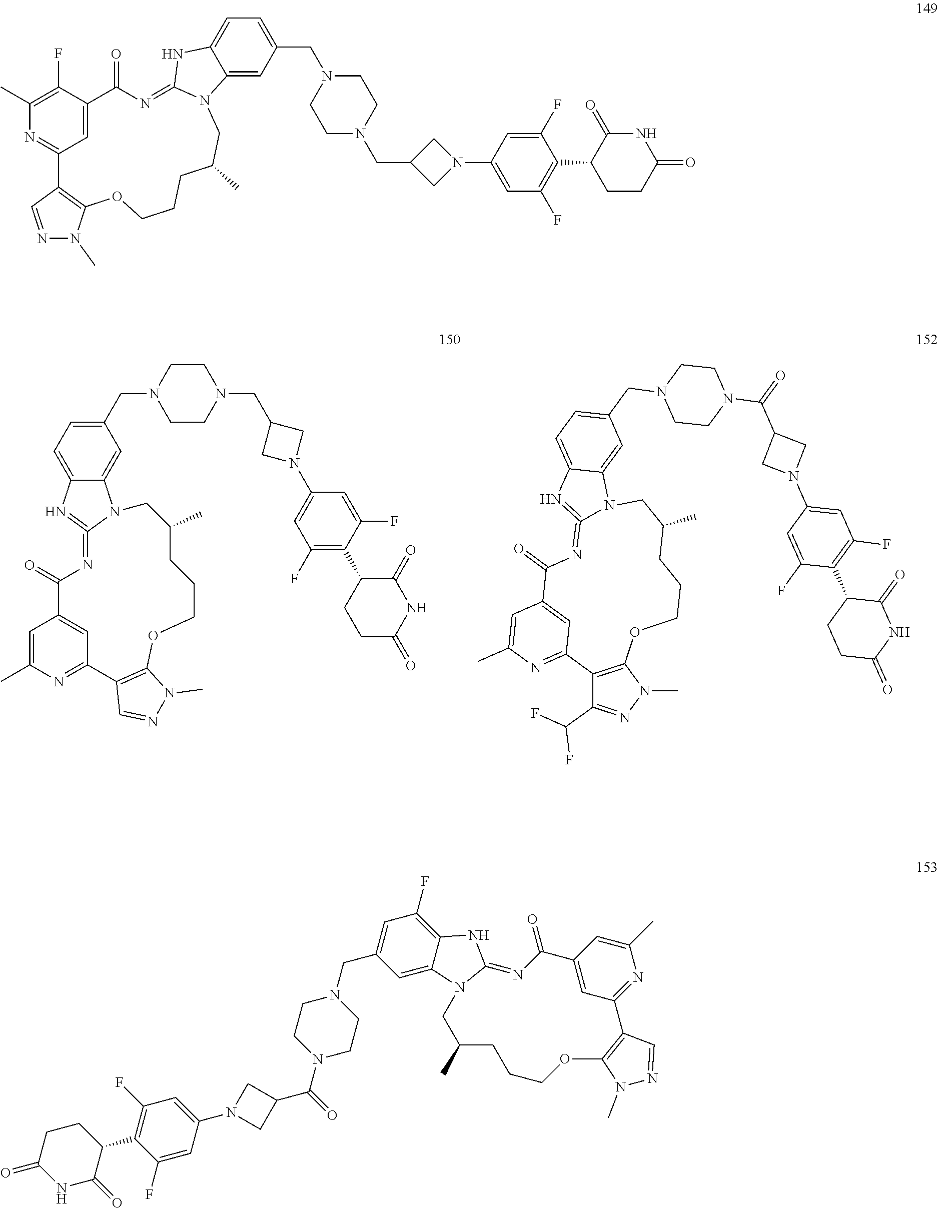

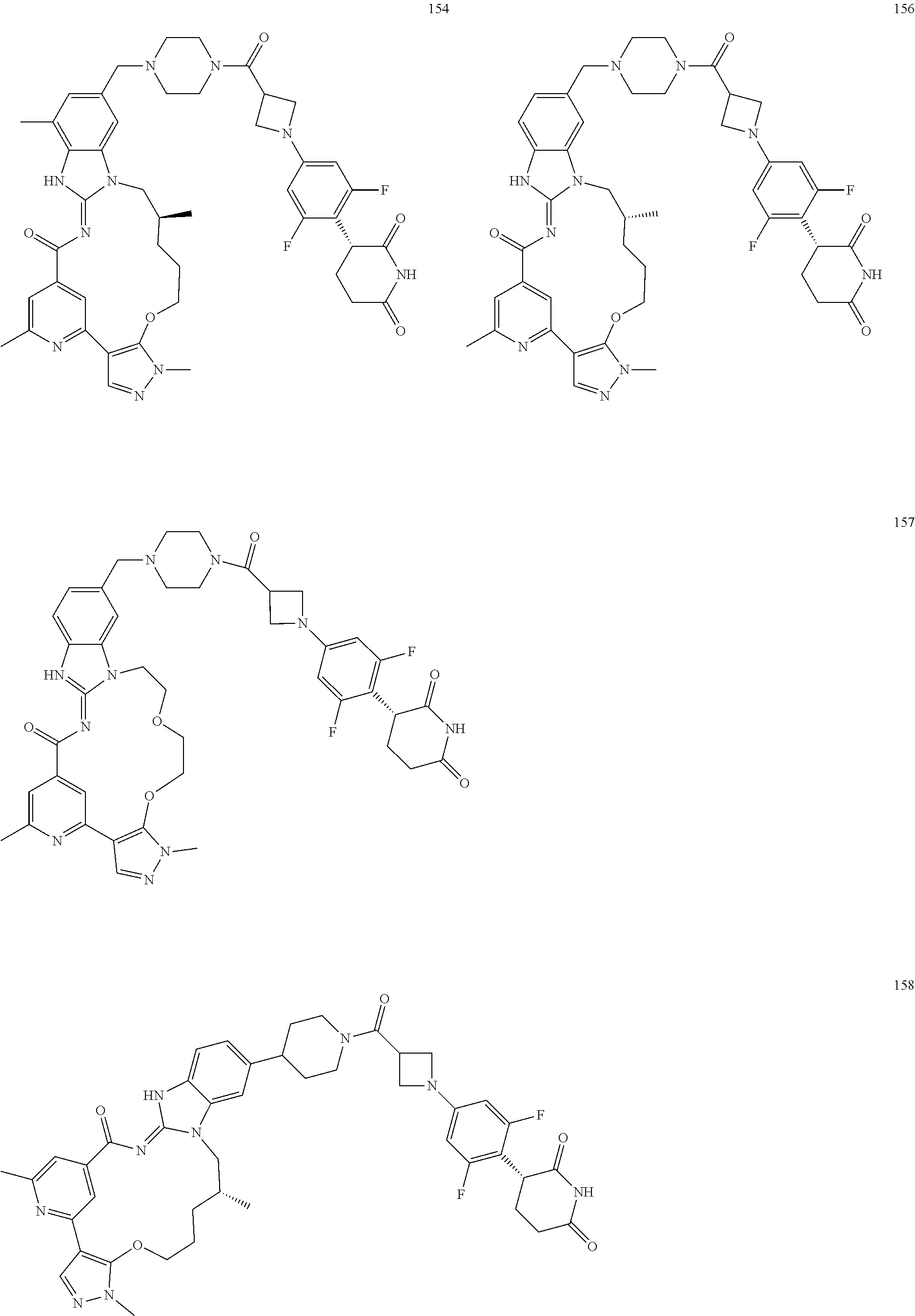

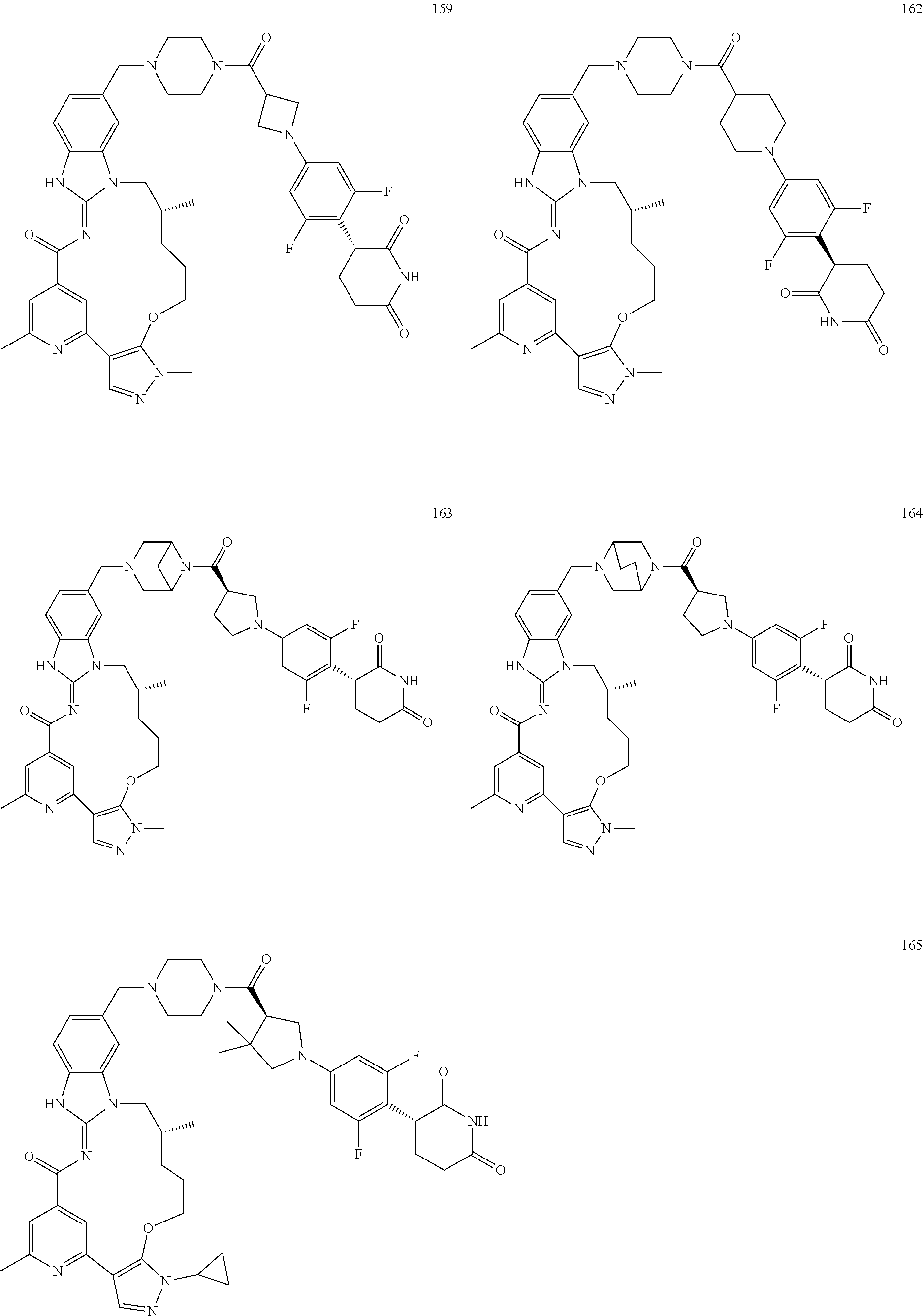

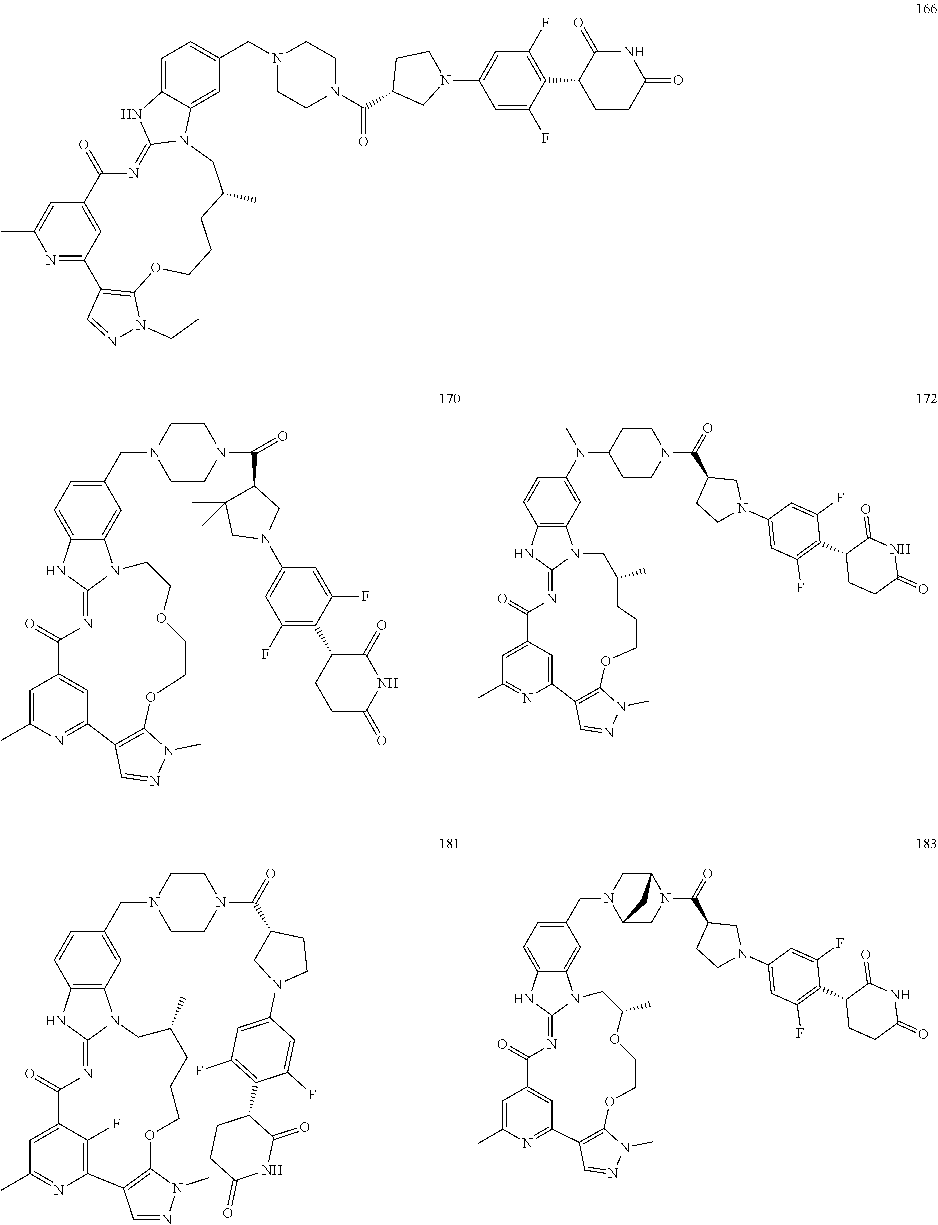

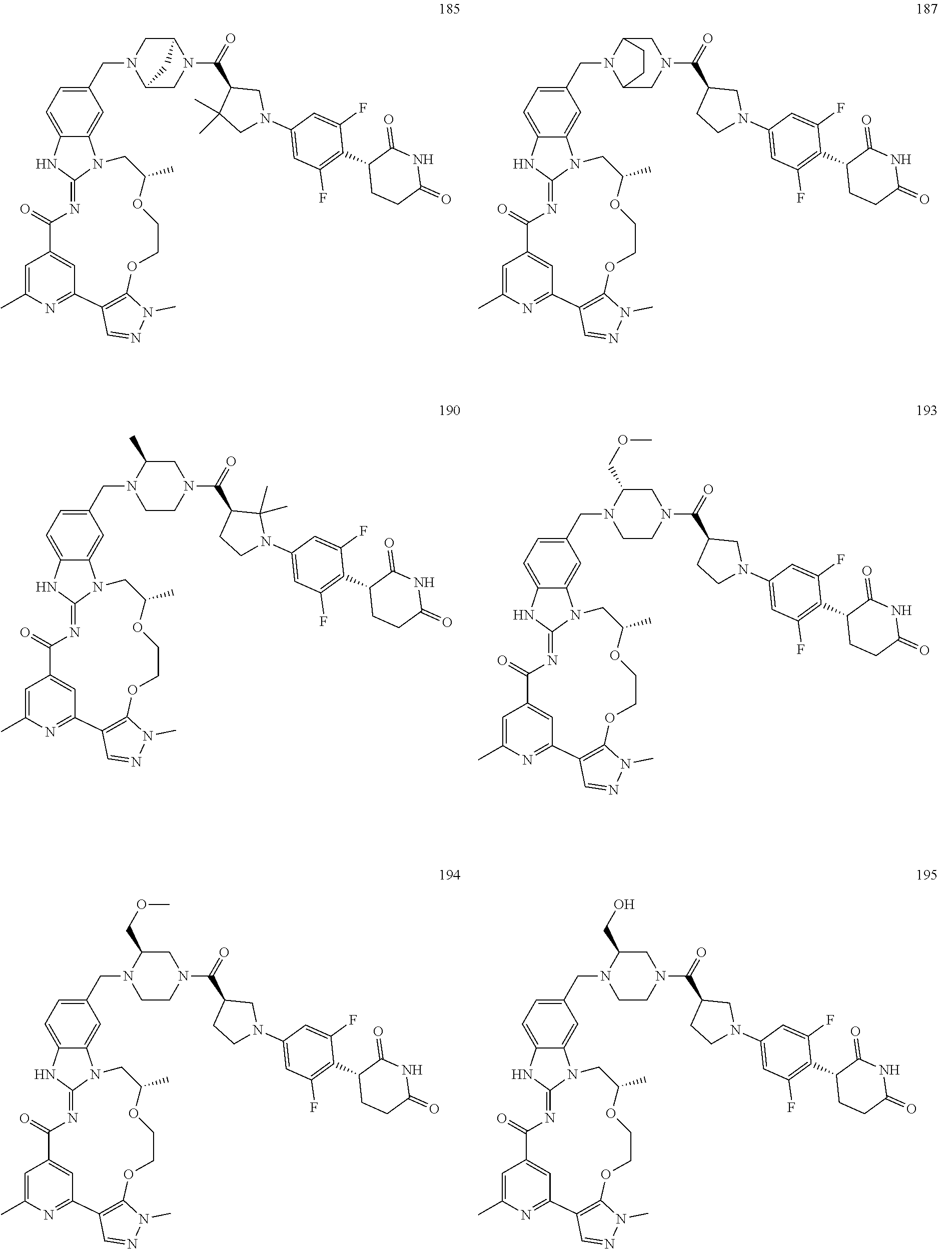

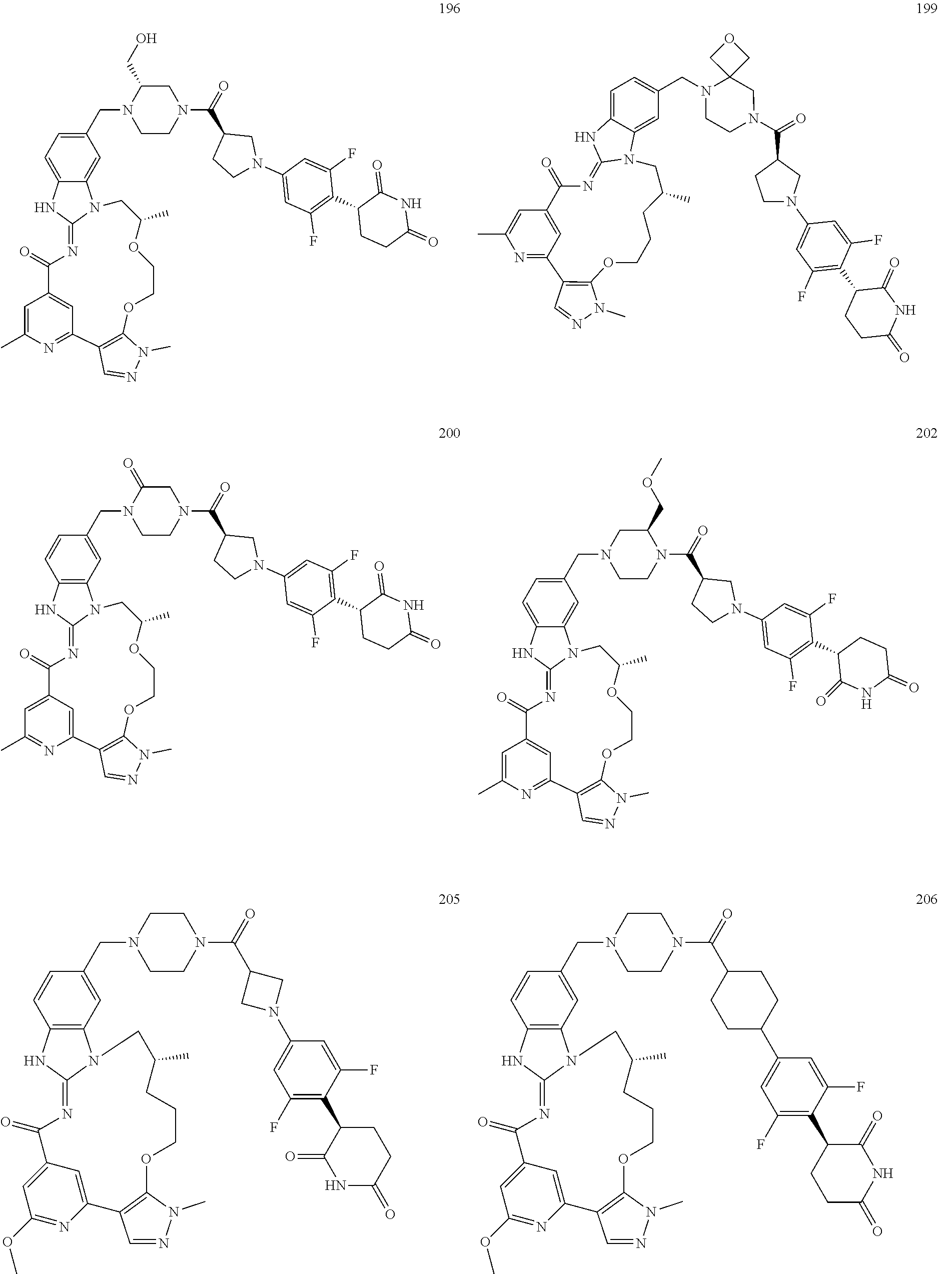

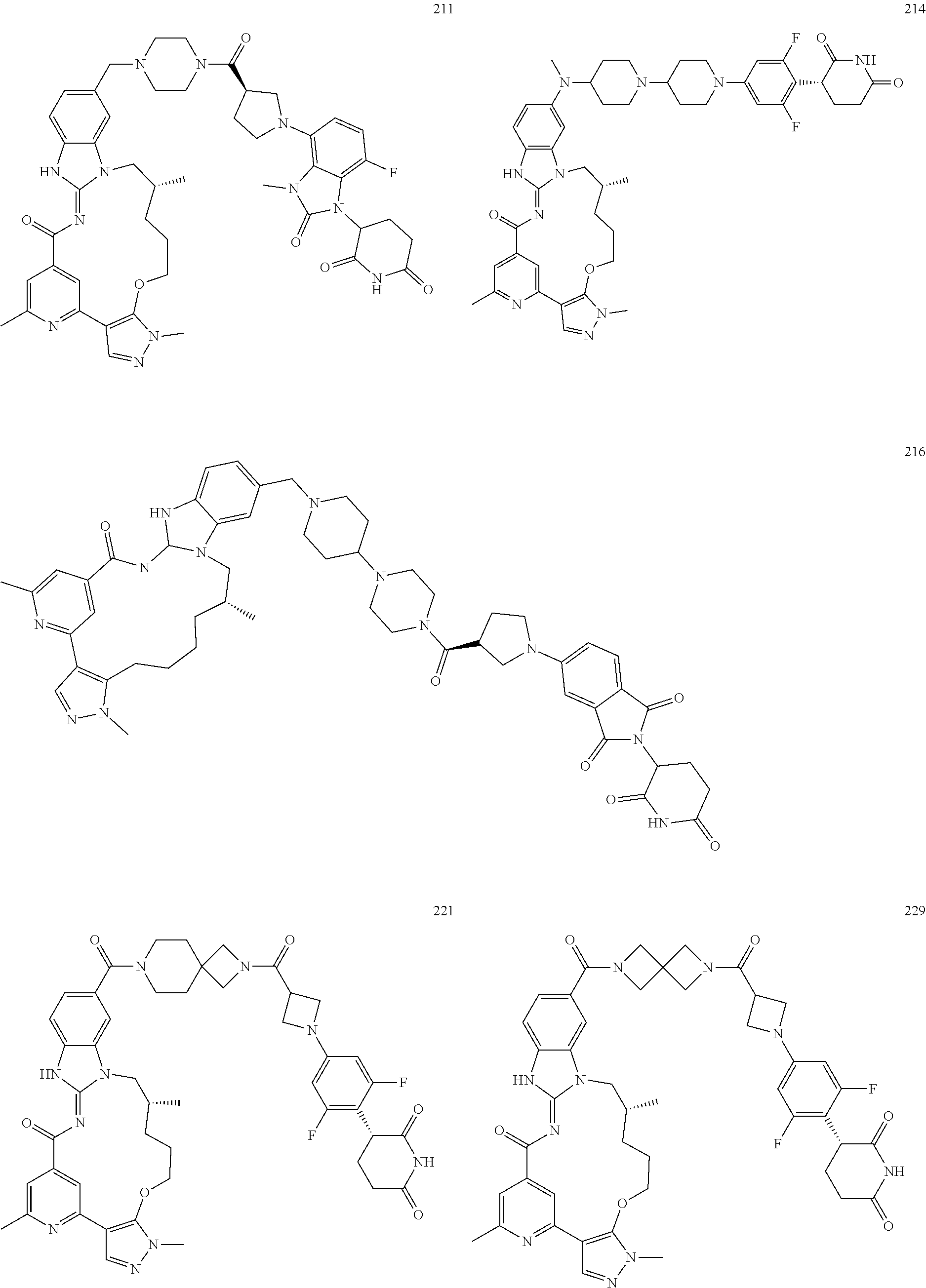

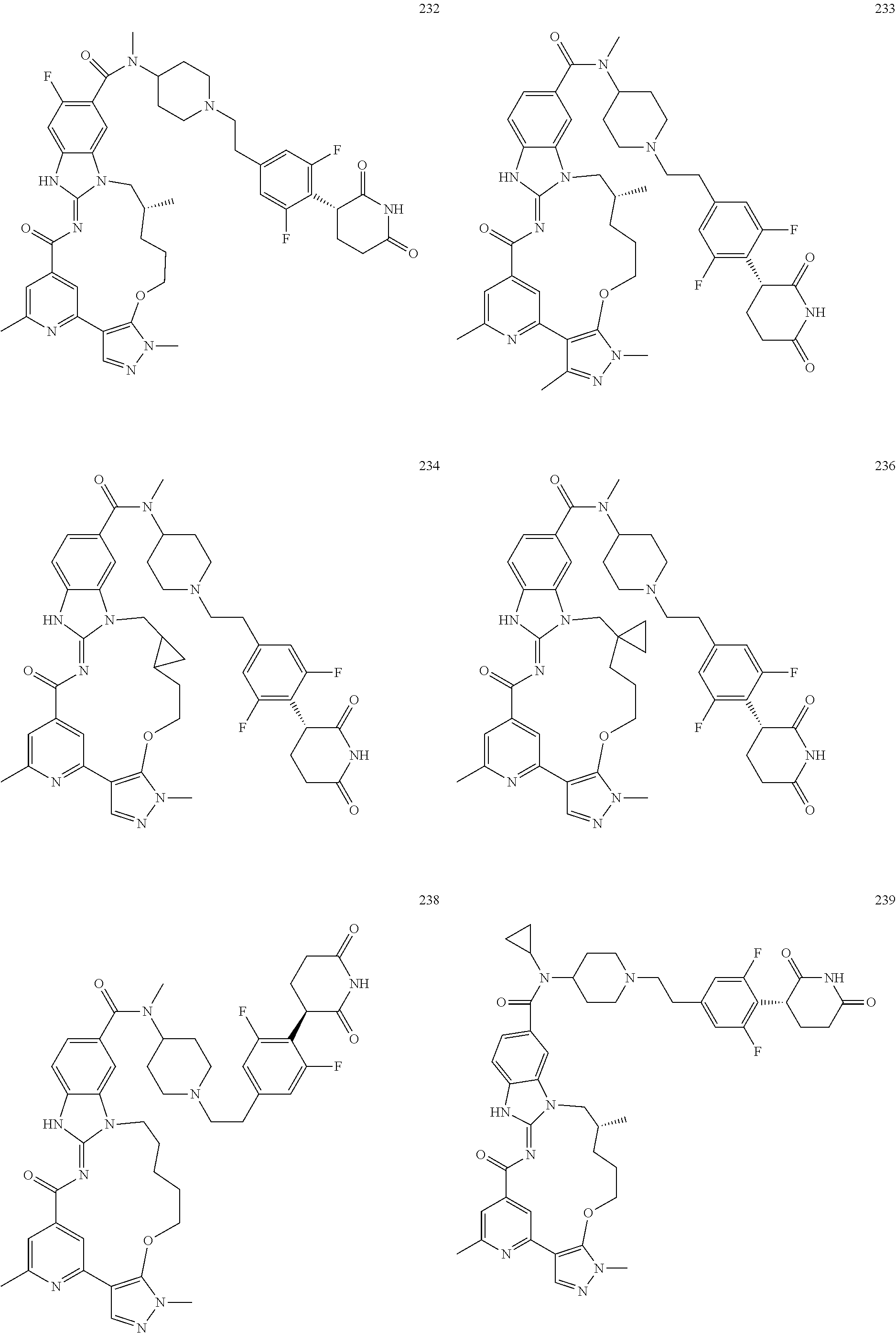

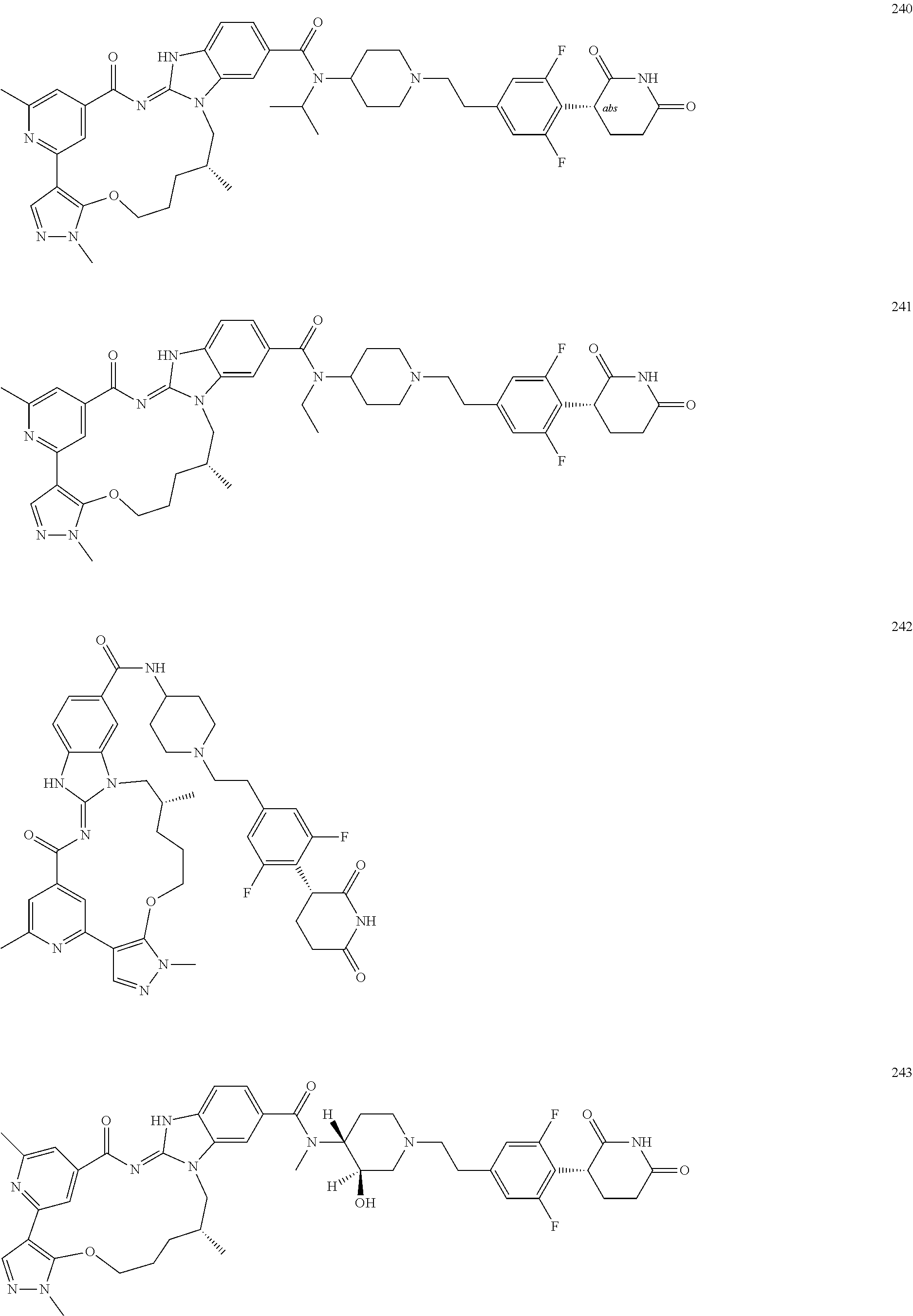 [SMILES: Cc1cc2cc(n1)-c1cnn(C)c1OCCC[C@@H](C)CN1/C(=N/C2=O)Nc2ccc(C(=O)N(C(C)C)C3CCN(CCc4cc(F)c([C@H]5CCC(=O)NC5=O)c(F)c4)CC3)cc21 ; CCN(C(=O)c1ccc2c(c1)N1C[C@H](C)CCCOc3c(cnn3C)-c3cc(cc(C)n3)C(=O)/N=C/1N2)C1CCN(CCc2cc(F)c([C@H]3CCC(=O)NC3=O)c(F)c2)CC1 ; Cc1cc2cc(n1)-c1cnn(C)c1OCCC[C@@H](C)CN1/C(=N/C2=O)Nc2ccc(C(=O)NC3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3O)cc21]

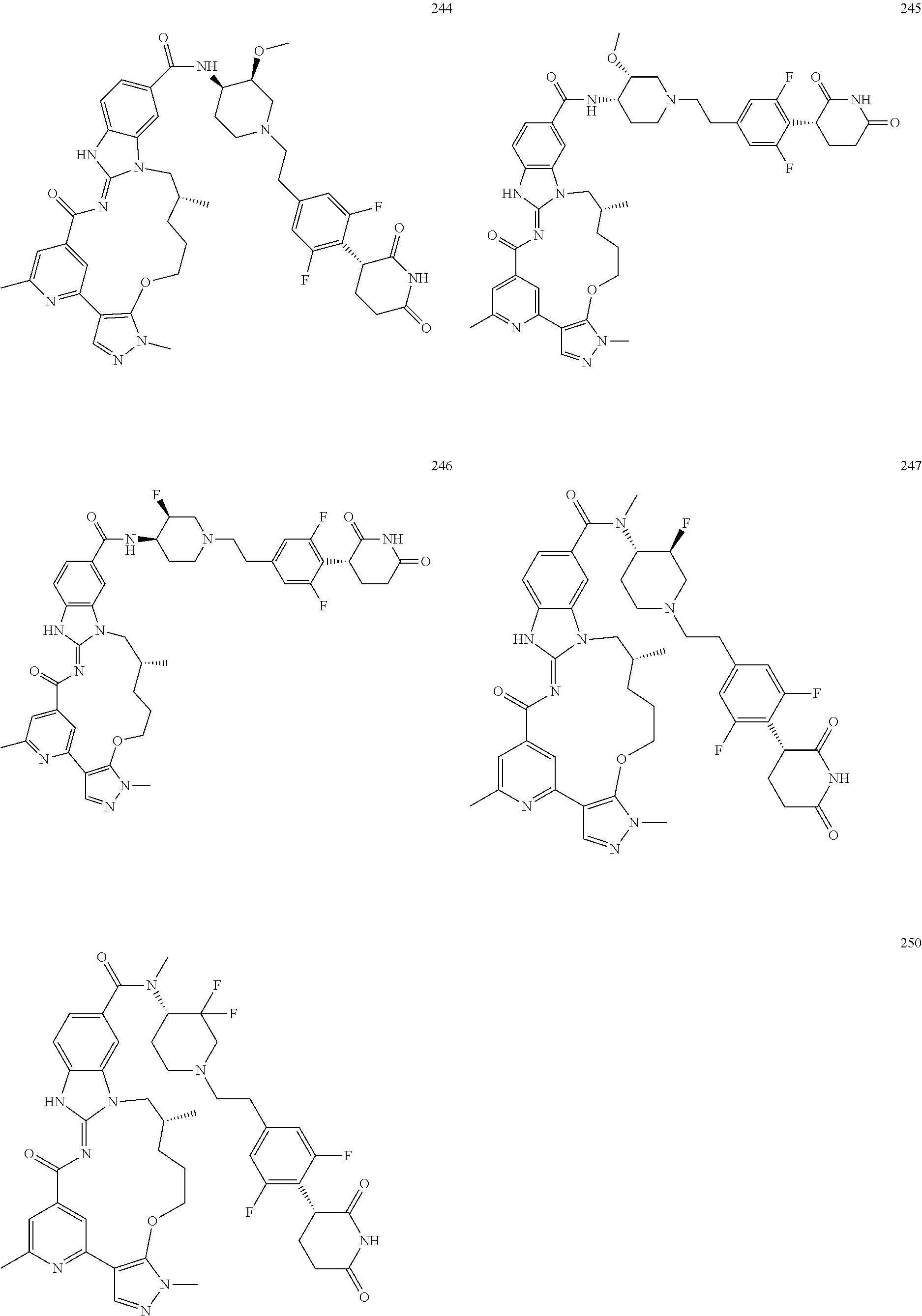

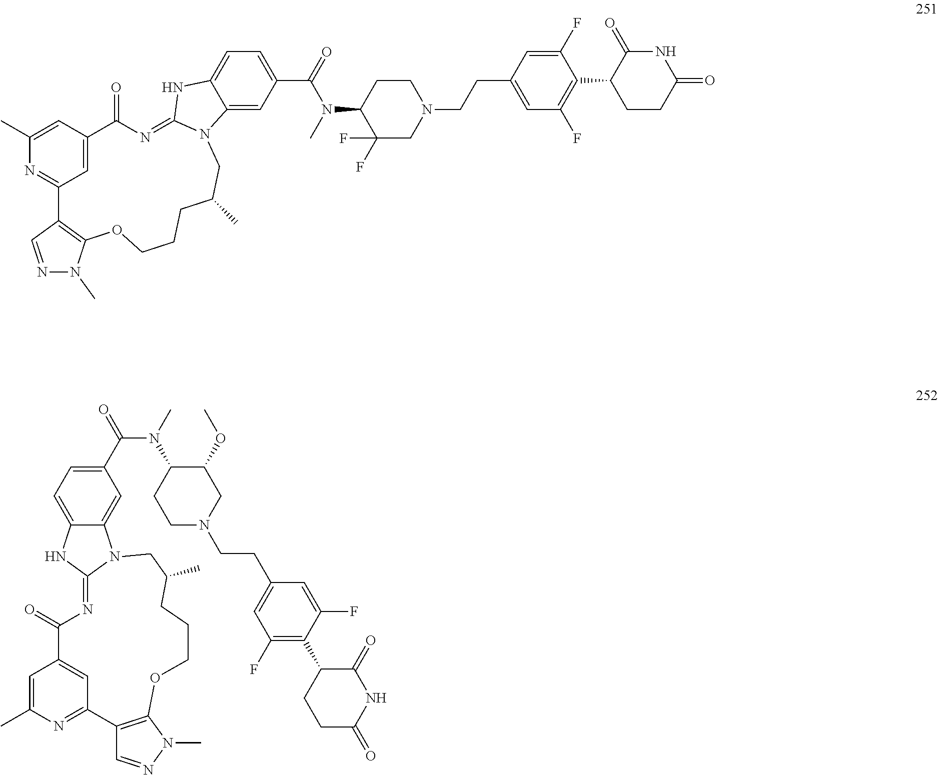 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)CC3(F)F)cc21 ; CO[C@@H]1CN(CCc2cc(F)c([C@H]3CCC(=O)NC3=O)c(F)c2)CC[C@@H]1N(C)C(=O)c1ccc2c(c1)N1C[C@H](C)CCCOc3c(cnn3C)-c3cc(cc(C)n3)C(=O)/N=C/1N2]

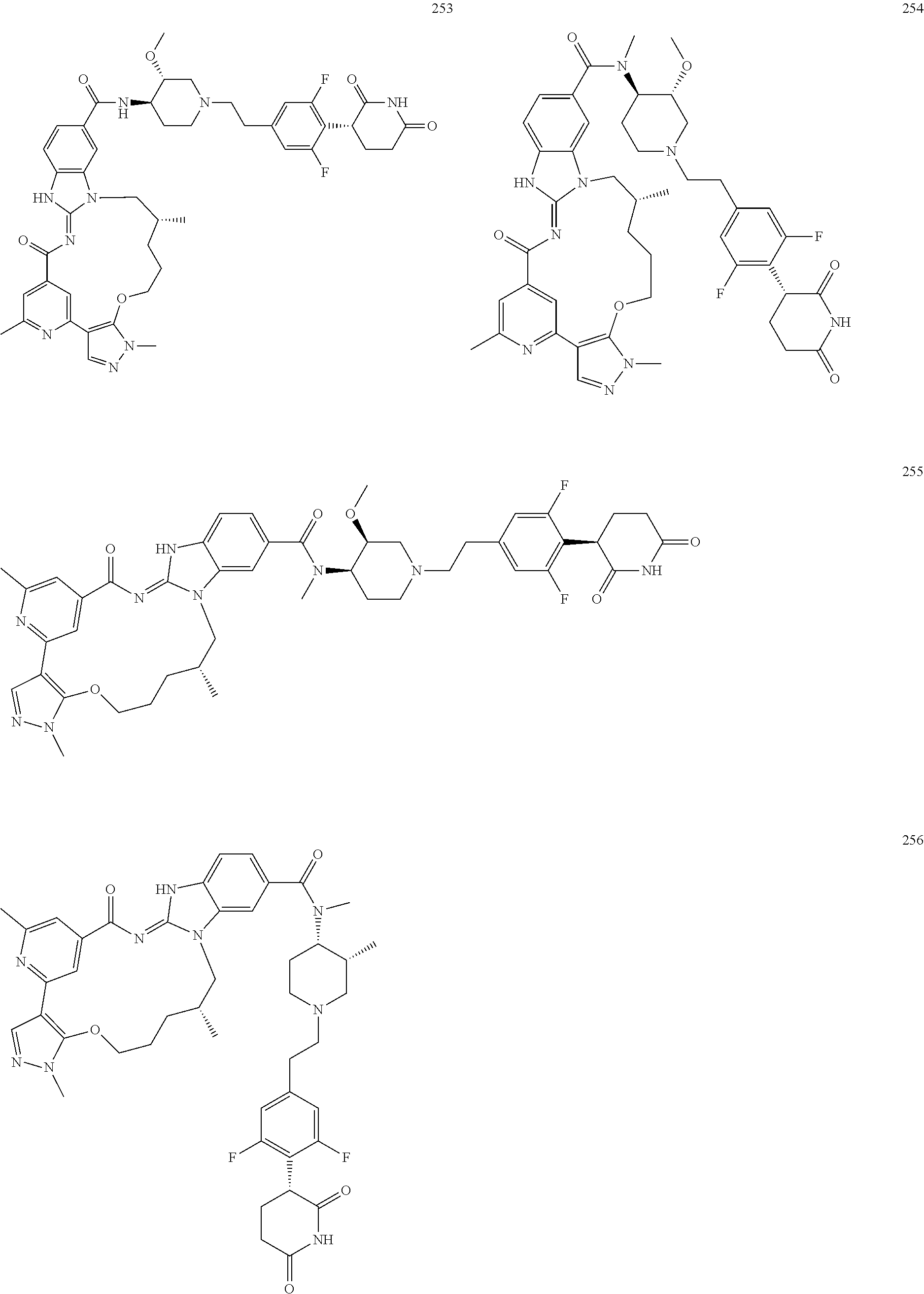

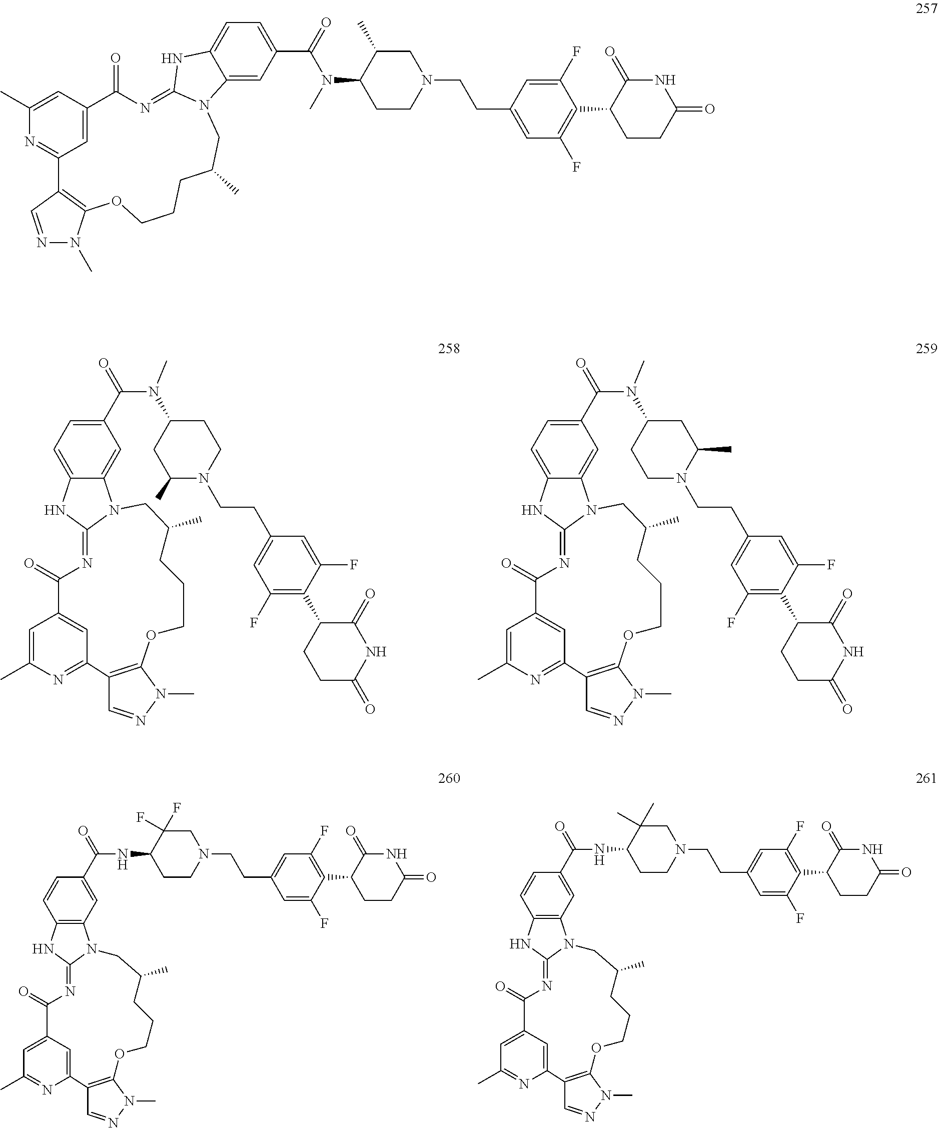

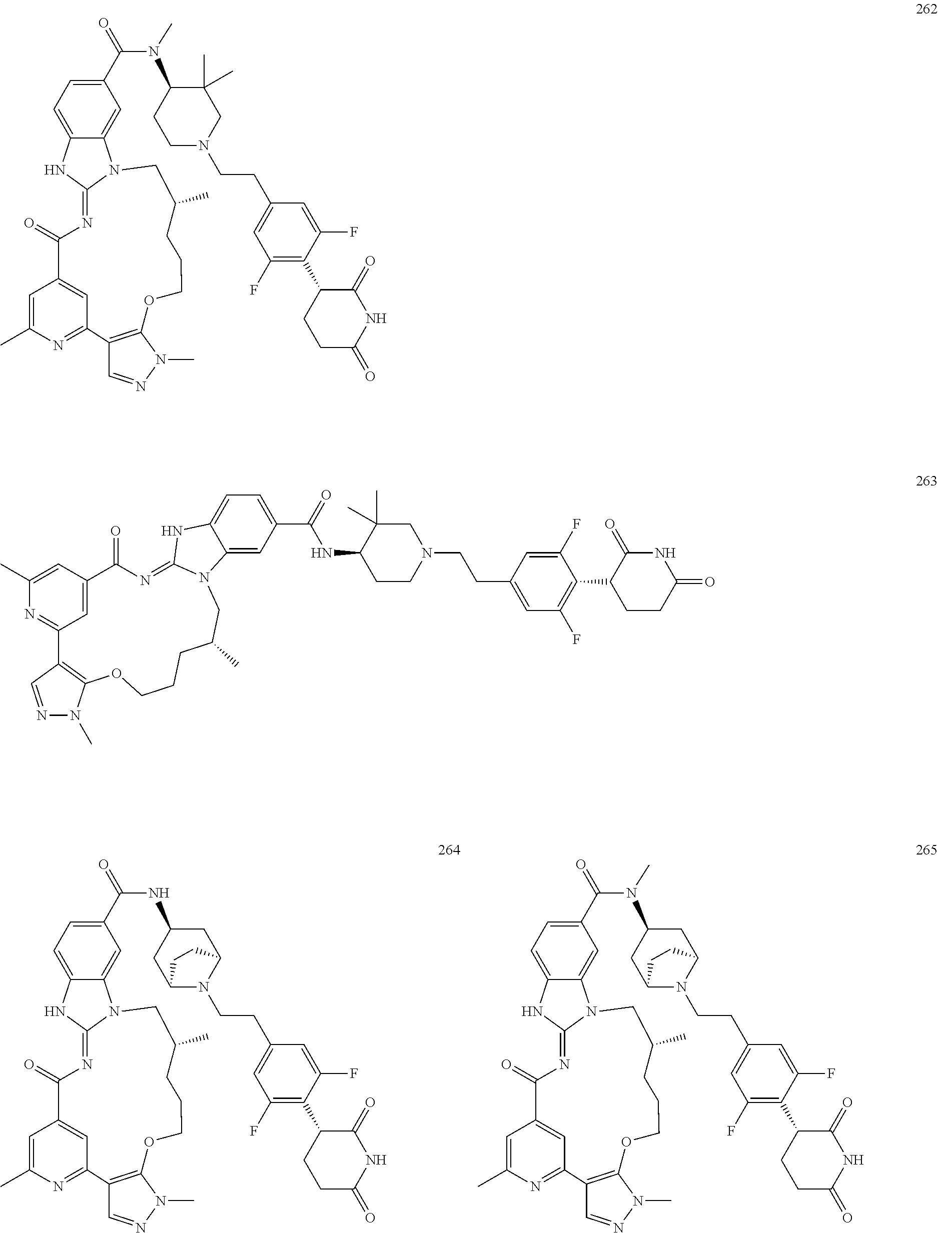

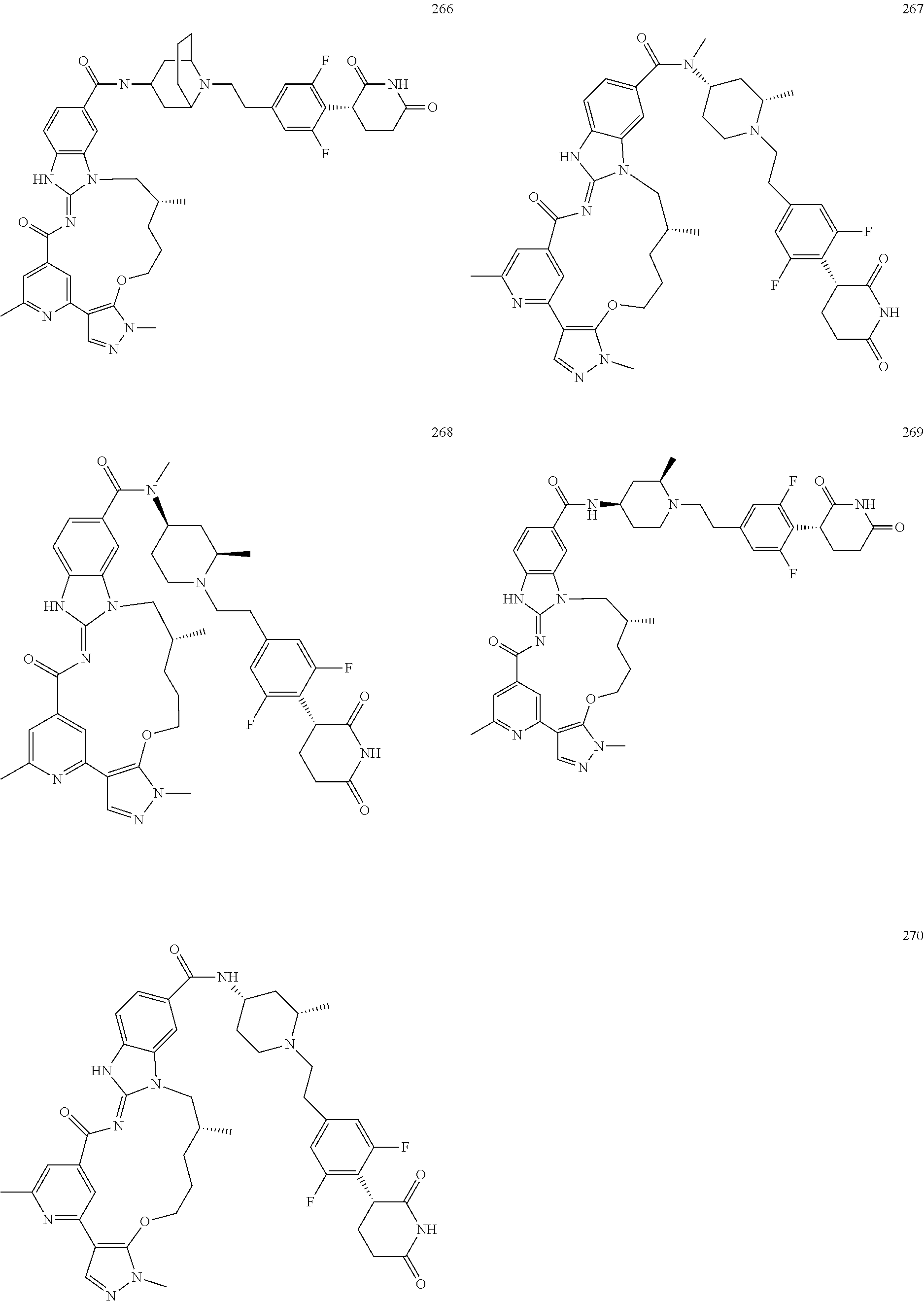

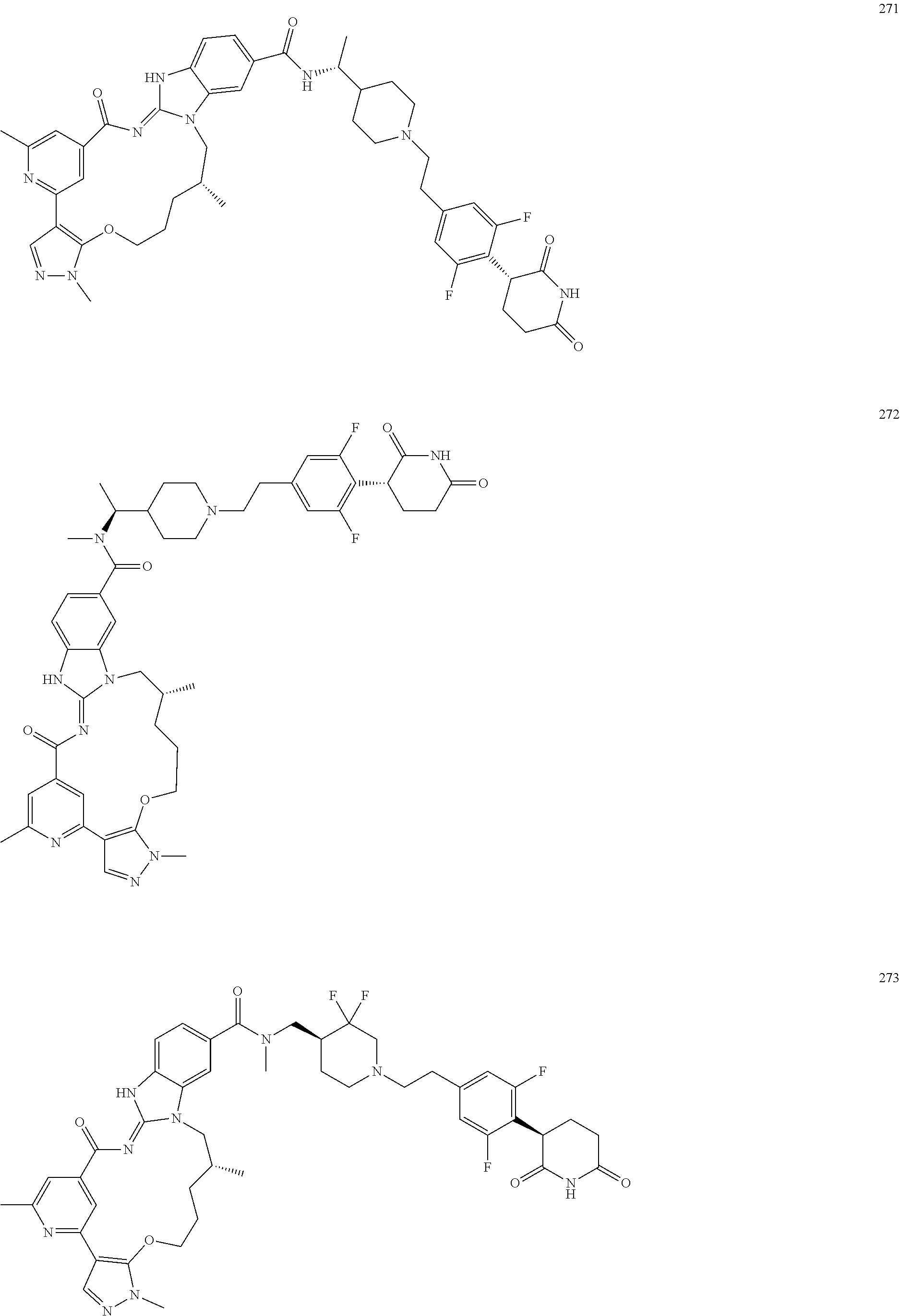 [SMILES: Cc1cc2cc(n1)-c1cnn(C)c1OCCC[C@@H](C)CN1/C(=N/C2=O)Nc2ccc(C(=O)N[C@H](C)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)[C@@H](C)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)C[C@@H]3CCN(CCc4cc(F)c([C@H]5CCC(=O)NC5=O)c(F)c4)CC3(F)F)cc21]

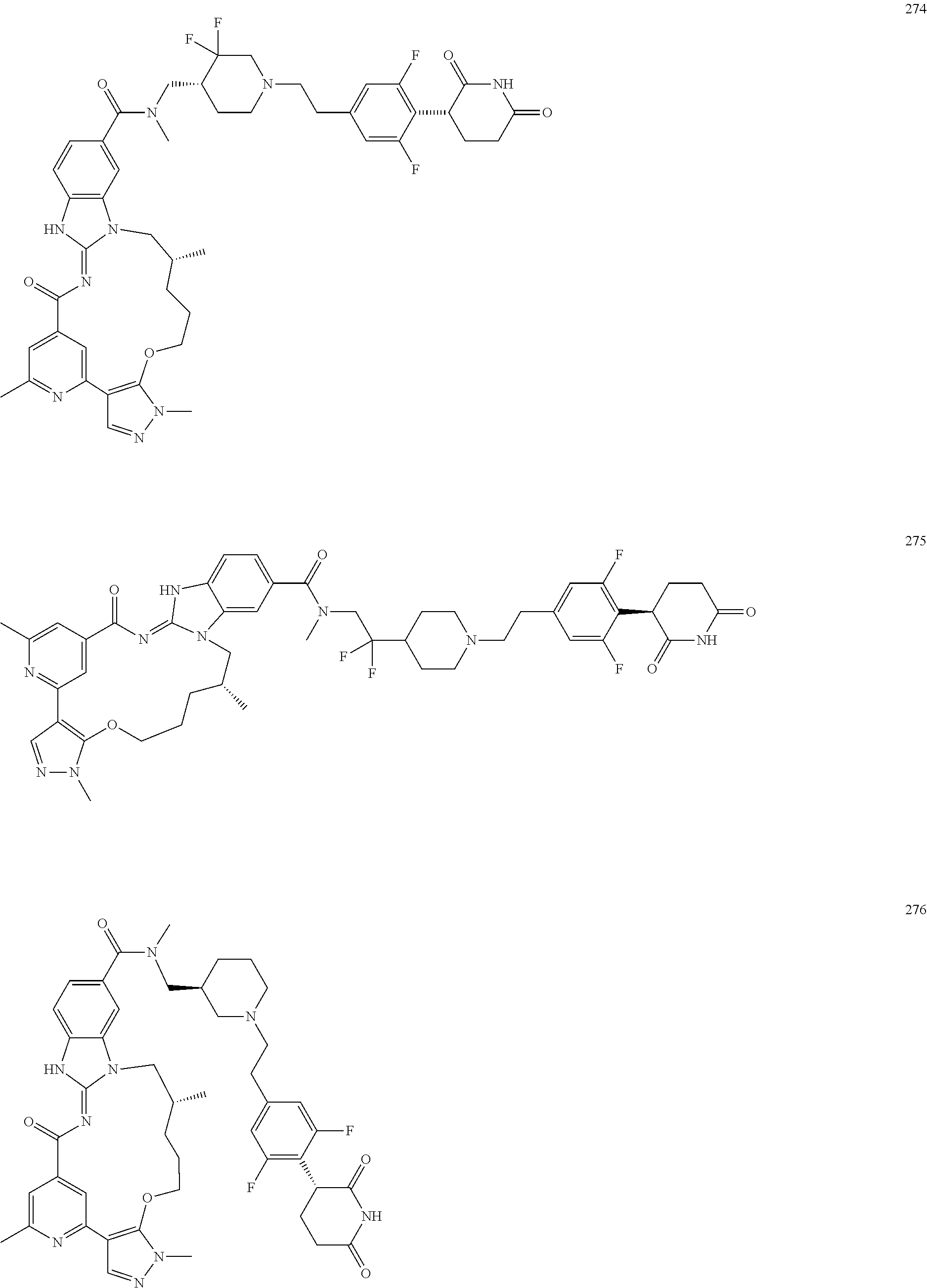

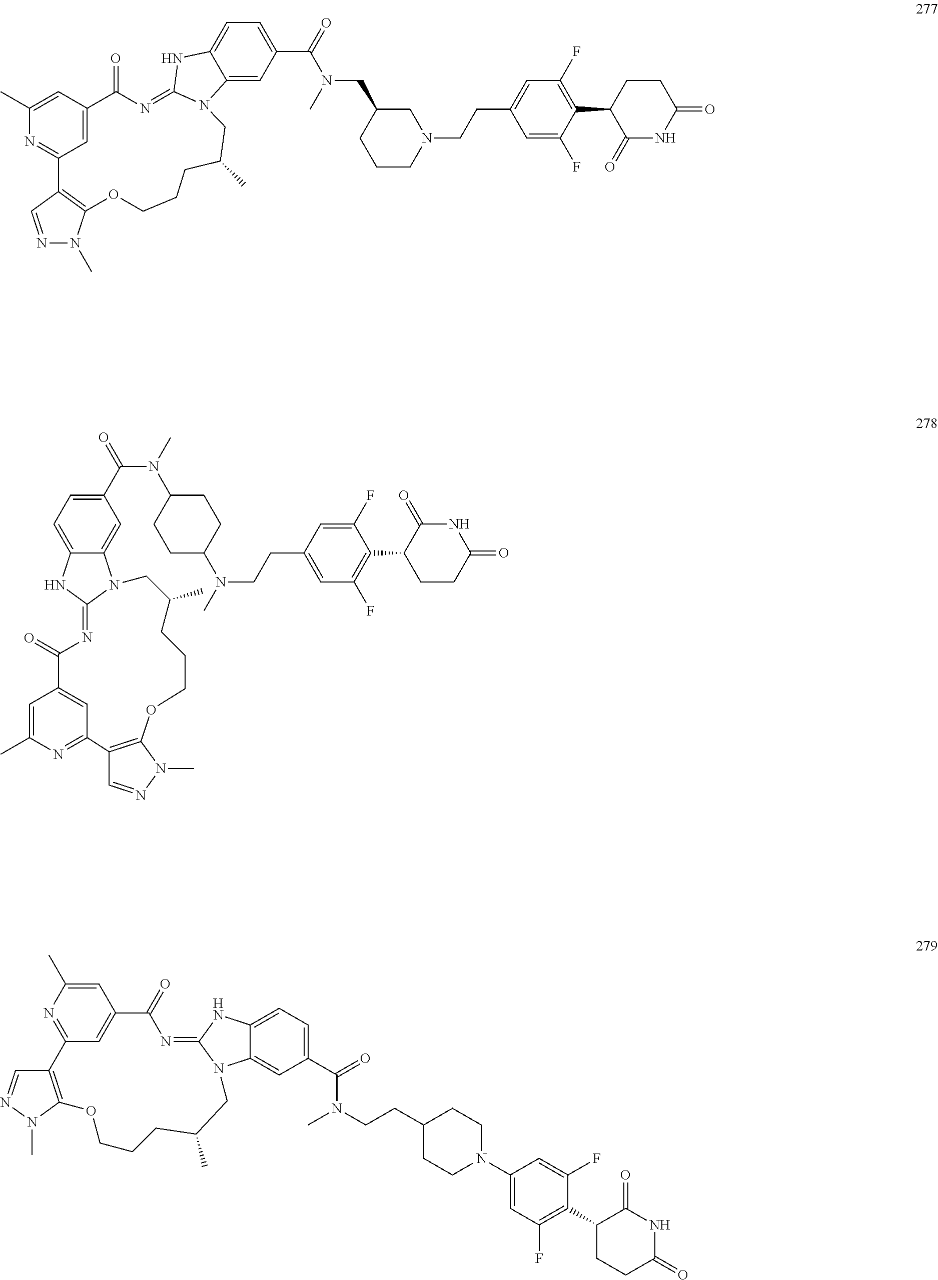 [SMILES: Cc1cc2cc(n1)-c1cnn(C)c1OCCC[C@@H](C)CN1/C(=N/C2=O)Nc2ccc(C(=O)N(C)C[C@@H]3CCCN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)C3CCC(N(C)CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)CCC3CCN(c4cc(F)c([C@H]5CCC(=O)NC5=O)c(F)c4)CC3)cc21]

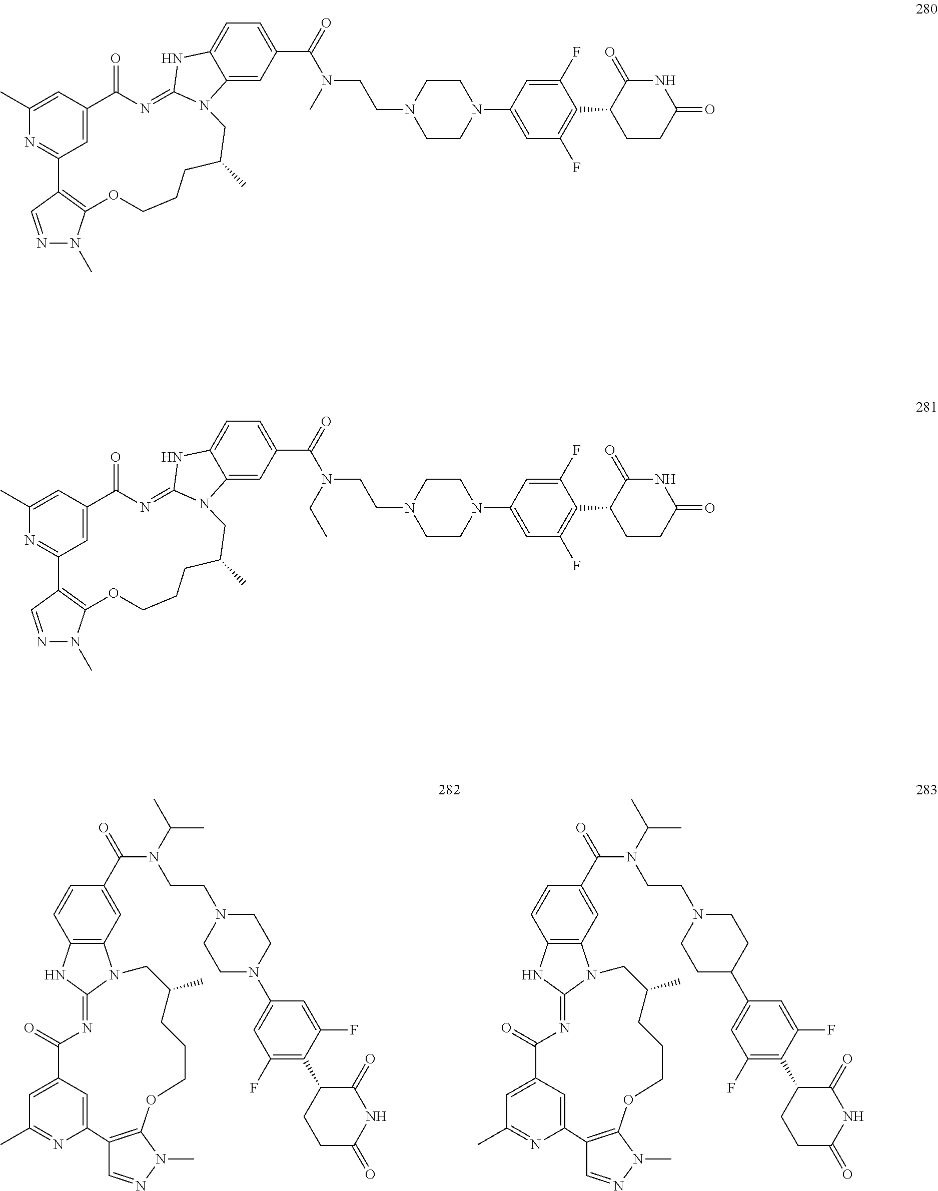 [SMILES: Cc1cc2cc(n1)-c1cnn(C)c1OCCC[C@@H](C)CN1/C(=N/C2=O)Nc2ccc(C(=O)N(C)CCN3CCN(c4cc(F)c([C@H]5CCC(=O)NC5=O)c(F)c4)CC3)cc21 ; CCN(CCN1CCN(c2cc(F)c([C@H]3CCC(=O)NC3=O)c(F)c2)CC1)C(=O)c1ccc2c(c1)N1C[C@H](C)CCCOc3c(cnn3C)-c3cc(cc(C)n3)C(=O)/N=C/1N2 ; Cc1cc2cc(n1)-c1cnn(C)c1OCCC[C@@H](C)CN1/C(=N/C2=O)Nc2ccc(C(=O)N(CCN3CCN(c4cc(F)c([C@H]5CCC(=O)NC5=O)c(F)c4)CC3)C(C)C)cc21 ; Cc1cc2cc(n1)-c1cnn(C)c1OCCC[C@@H](C)CN1/C(=N/C2=O)Nc2ccc(C(=O)N(CCN3CCC(c4cc(F)c([C@H]5CCC(=O)NC5=O)c(F)c4)CC3)C(C)C)cc21]

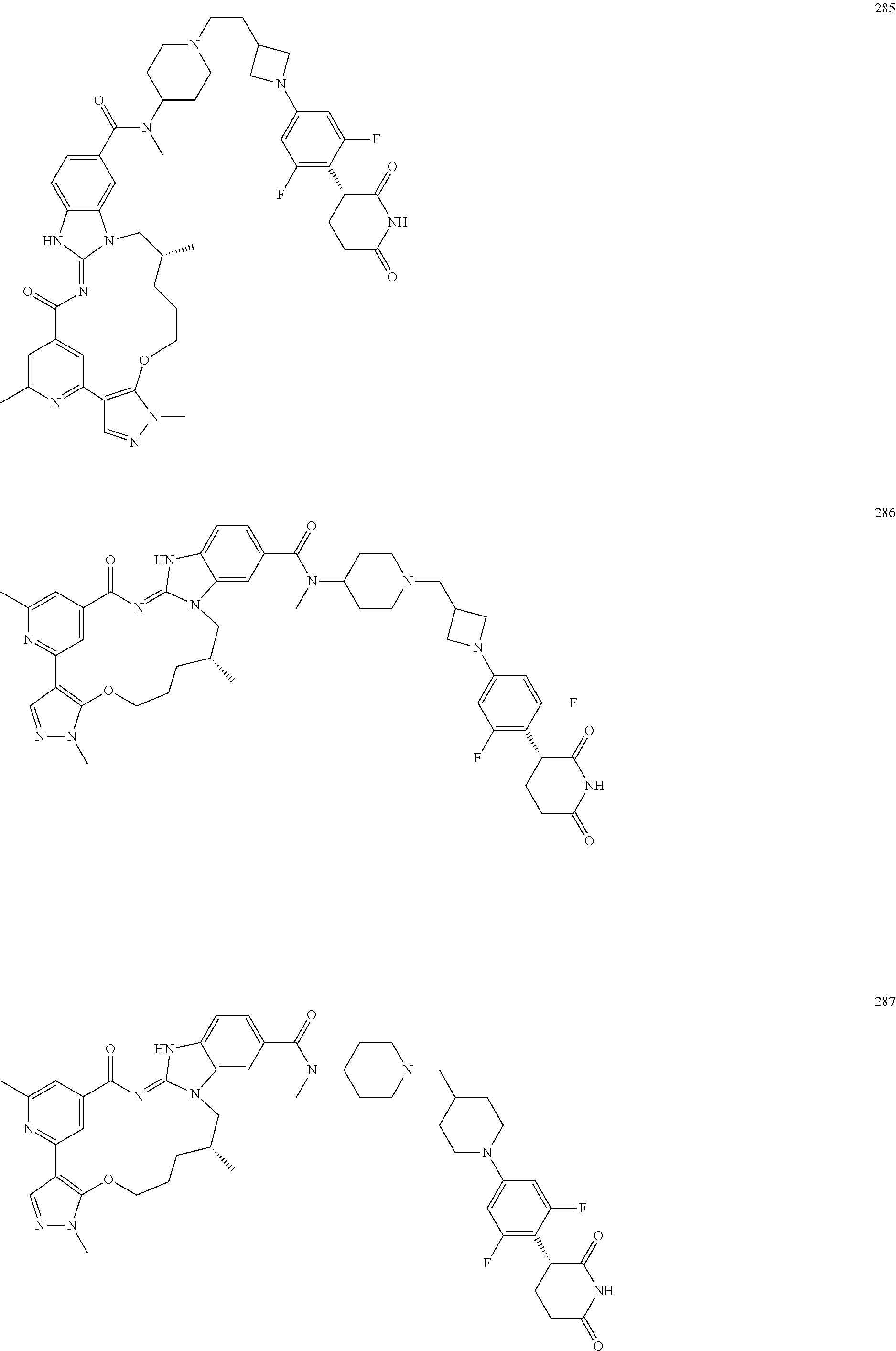 [SMILES: Cc1cc2cc(n1)-c1cnn(C)c1OCCC[C@@H](C)CN1/C(=N/C2=O)Nc2ccc(C(=O)N(C)C3CCN(CCC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C(=O)N(C)C3CCN(CC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C(=O)N(C)C3CCN(CC4CCN(c5cc(F)c([C@H]6CCC(=O)NC6=O)c(F)c5)CC4)CC3)cc21]

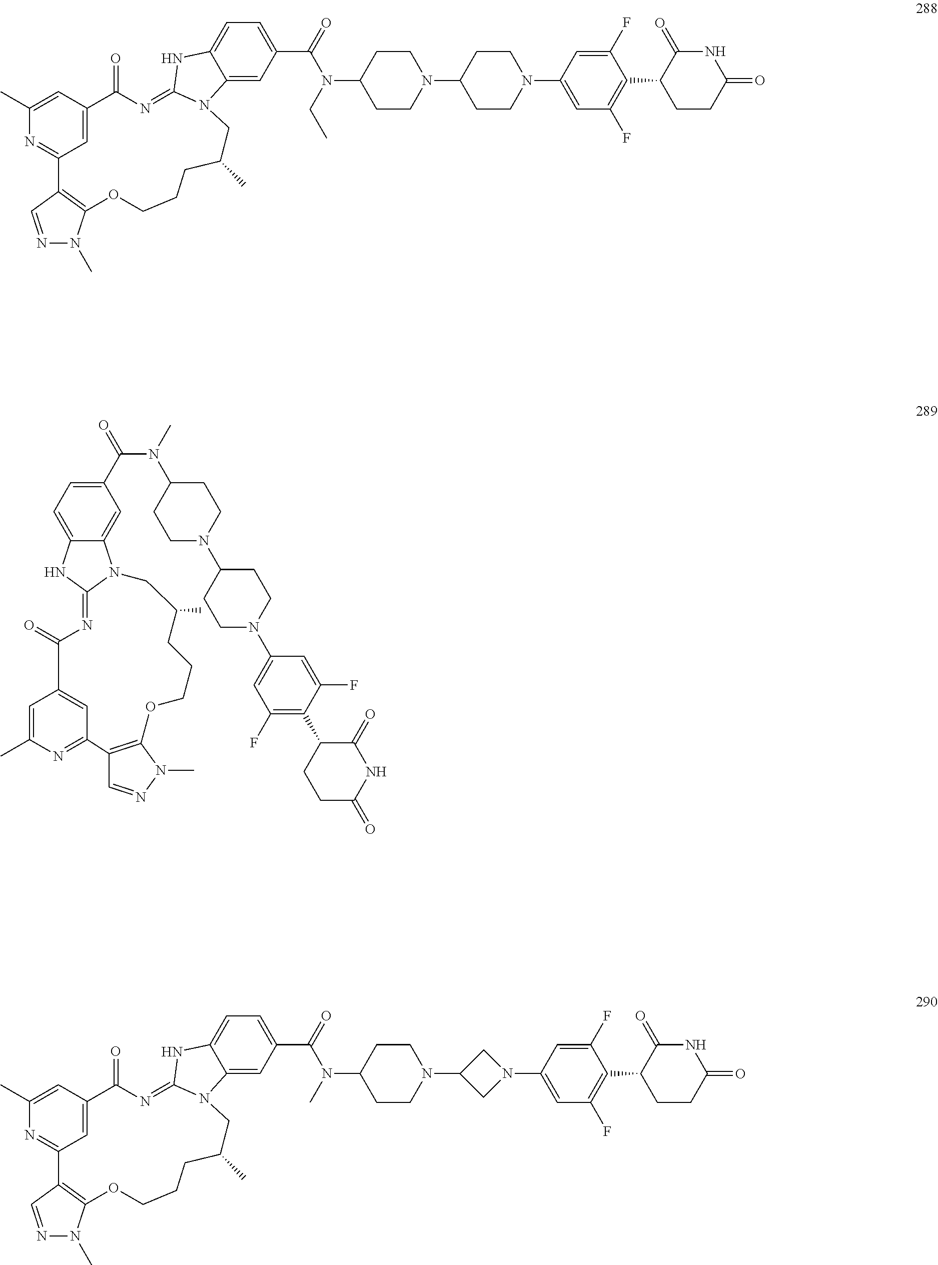 [SMILES: CCN(C(=O)c1ccc2c(c1)N1C[C@H](C)CCCOc3c(cnn3C)-c3cc(cc(C)n3)C(=O)/N=C/1N2)C1CCN(C2CCN(c3cc(F)c([C@H]4CCC(=O)NC4=O)c(F)c3)CC2)CC1 ; Cc1cc2cc(n1)-c1cnn(C)c1OCCC[C@@H](C)CN1/C(=N/C2=O)Nc2ccc(C(=O)N(C)C3CCN(C4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(C(=O)N(C)C3CCN(C4CN(c5cc(F)c([C@H]6CCC(=O)NC6=O)c(F)c5)C4)CC3)cc21]

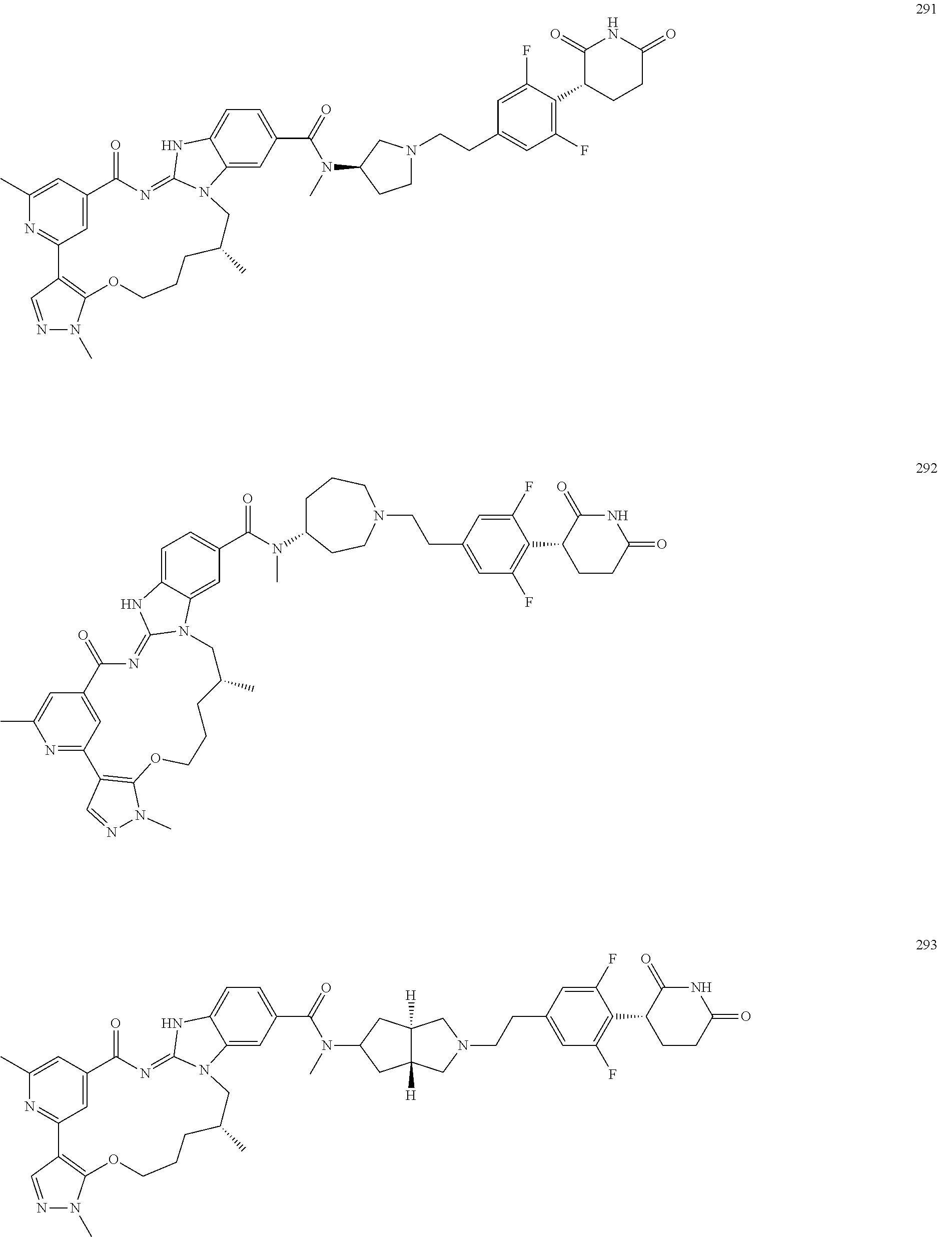 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)[C@@H]3CCCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)C3C[C@H]4CN(CCc5cc(F)c([C@H]6CCC(=O)NC6=O)c(F)c5)C[C@@H]4C3)cc21]

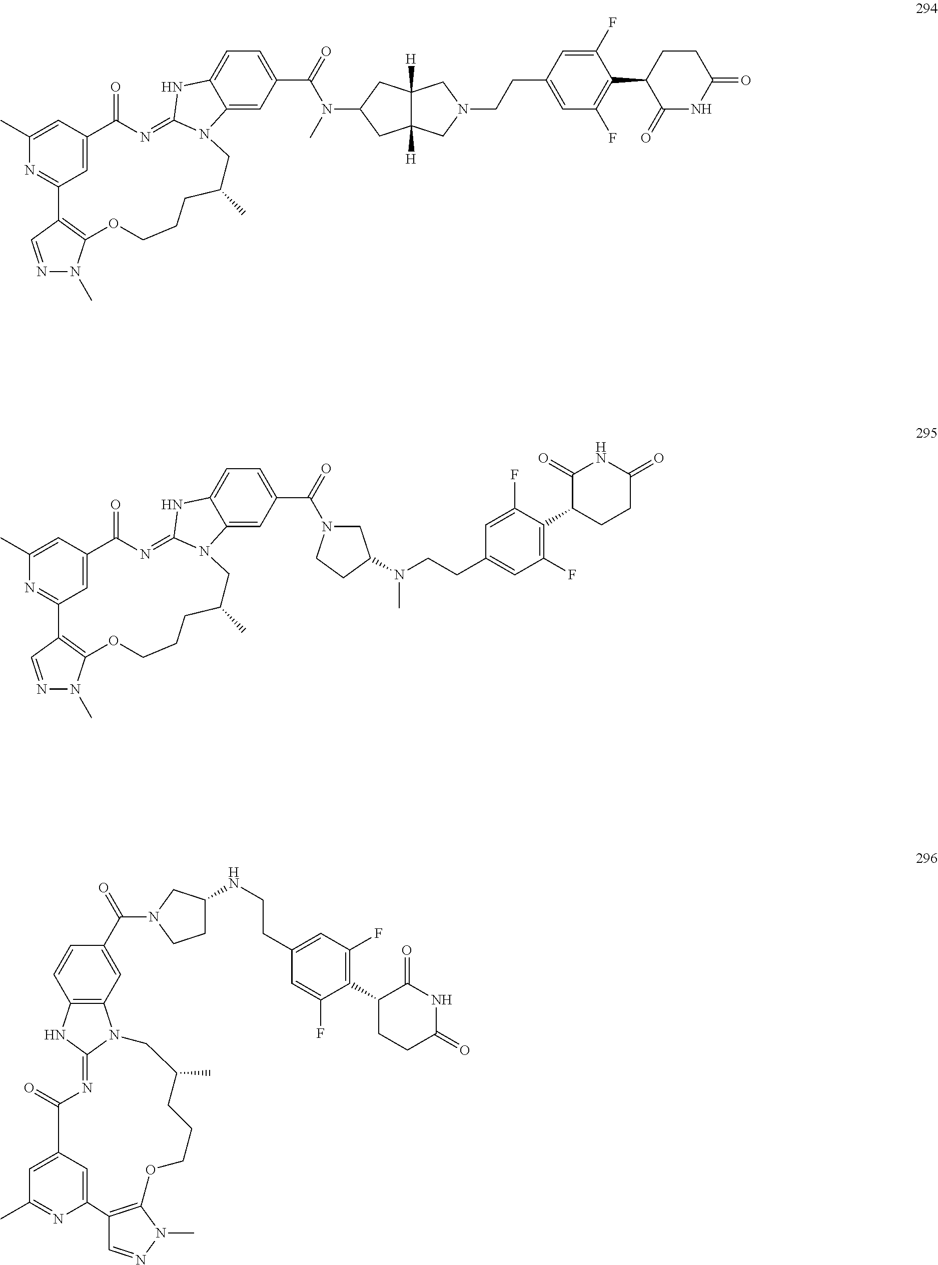

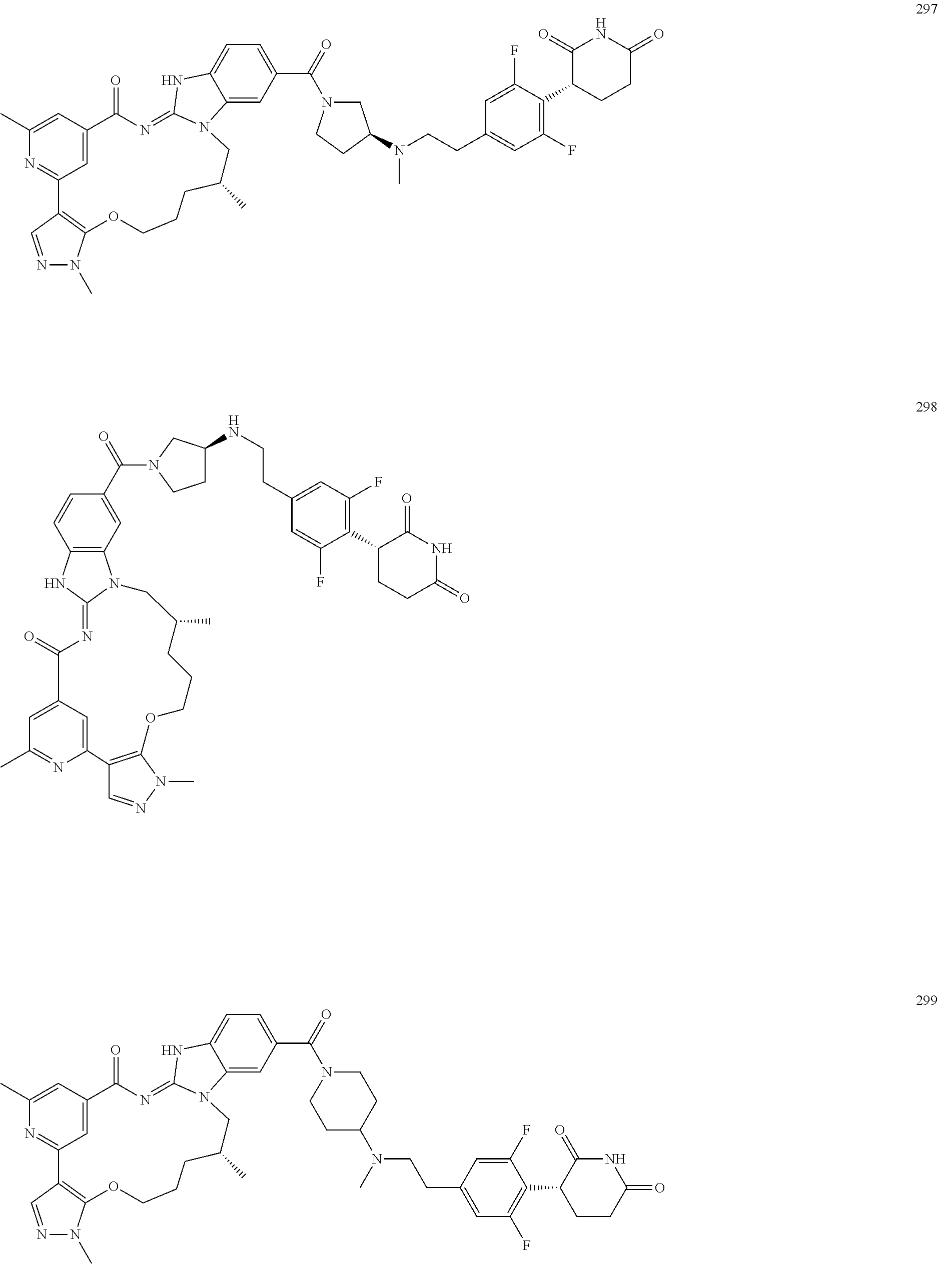 [SMILES: Cc1cc2cc(n1)-c1cnn(C)c1OCCC[C@@H](C)CN1/C(=N/C2=O)Nc2ccc(C(=O)N3CC[C@H](N(C)CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N3CC[C@H](NCCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N3CCC(N(C)CCc4cc(F)c([C@H]5CCC(=O)NC5=O)c(F)c4)CC3)cc21]

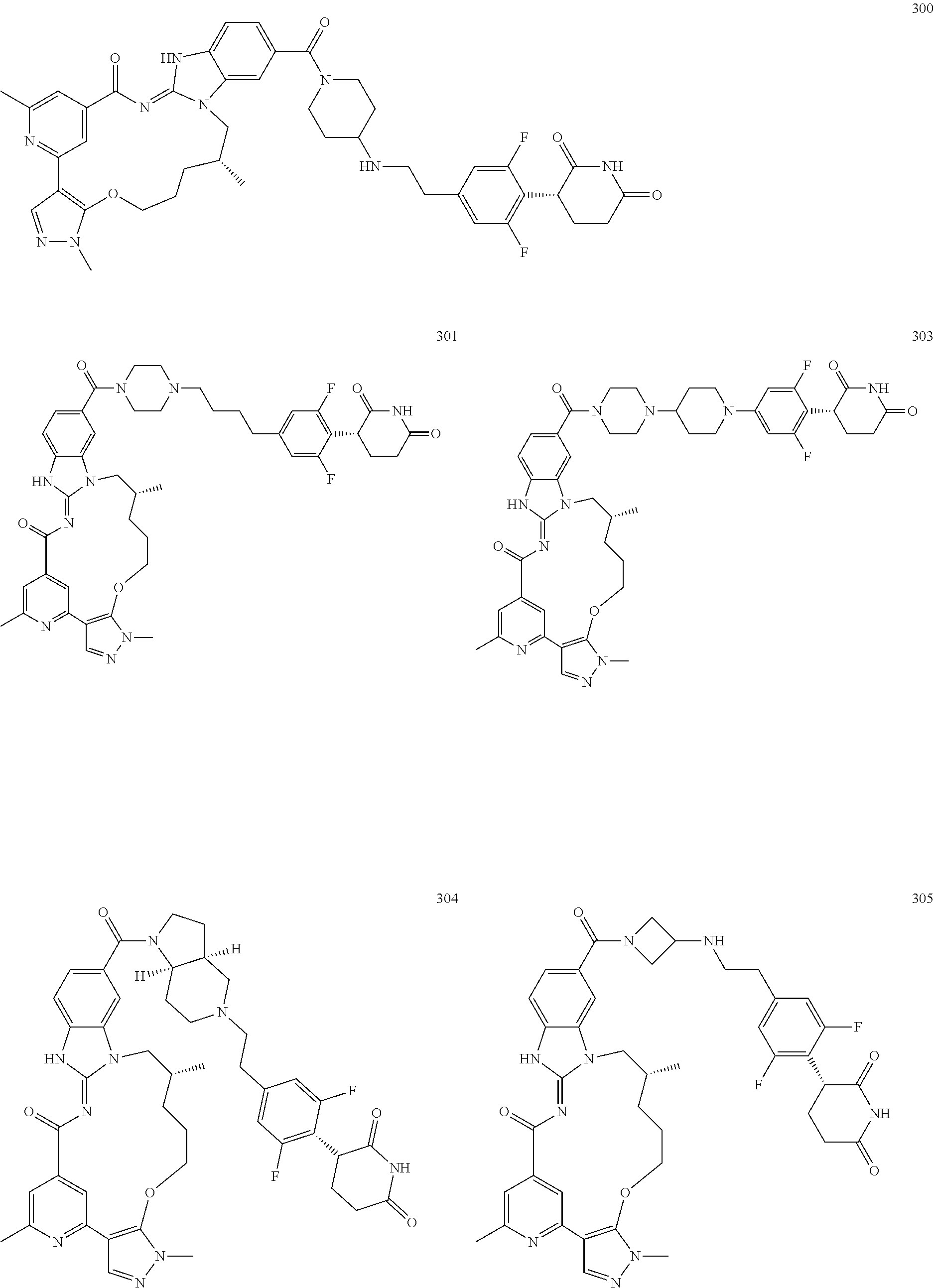

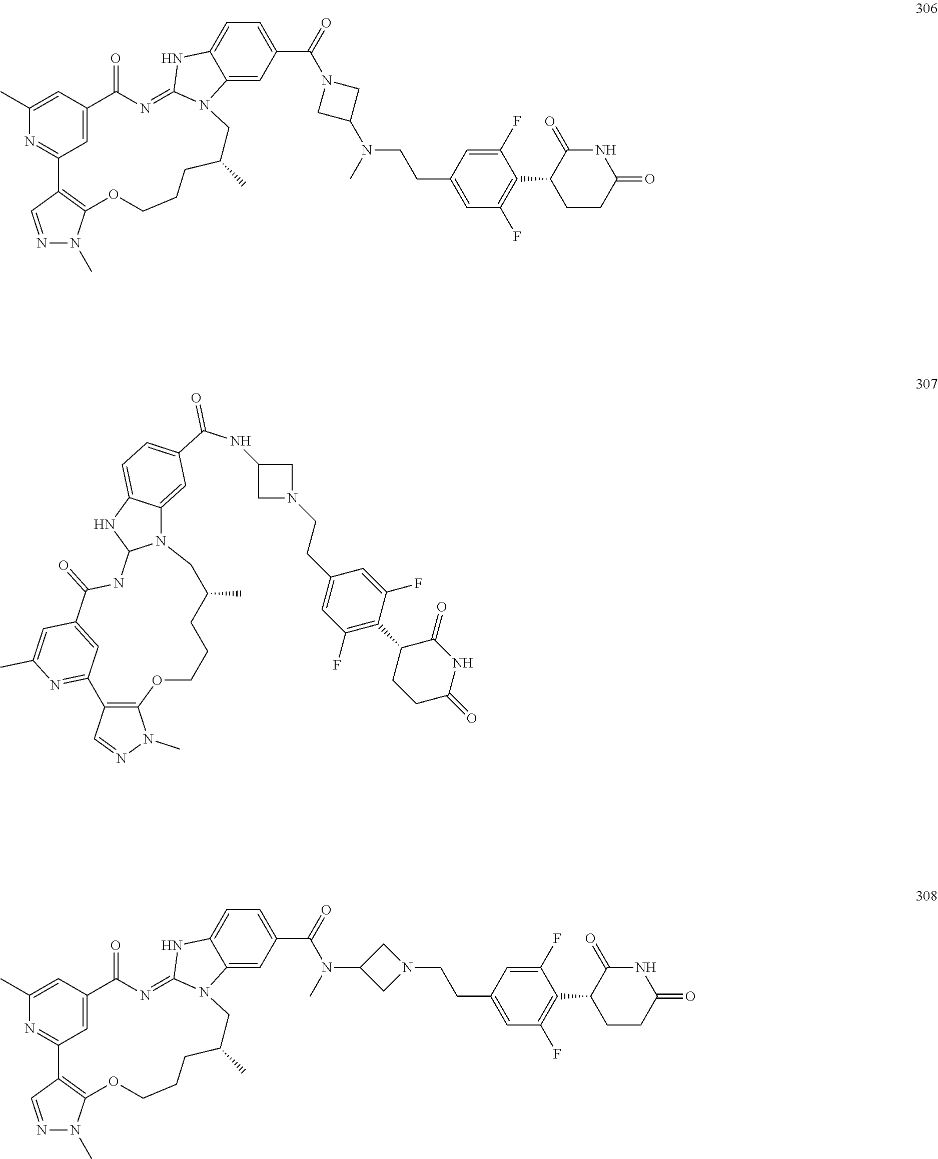 [SMILES: Cc1cc2cc(n1)-c1cnn(C)c1OCCC[C@@H](C)CN1/C(=N/C2=O)Nc2ccc(C(=O)N3CC(N(C)CCc4cc(F)c([C@H]5CCC(=O)NC5=O)c(F)c4)C3)cc21 ; Cc1cc2cc(n1)-c1cnn(C)c1OCCC[C@@H](C)CN1c3cc(C(=O)NC4CN(CCc5cc(F)c([C@H]6CCC(=O)NC6=O)c(F)c5)C4)ccc3NC1NC2=O ; Cc1cc2cc(n1)-c1cnn(C)c1OCCC[C@@H](C)CN1/C(=N/C2=O)Nc2ccc(C(=O)N(C)C3CN(CCc4cc(F)c([C@H]5CCC(=O)NC5=O)c(F)c4)C3)cc21]

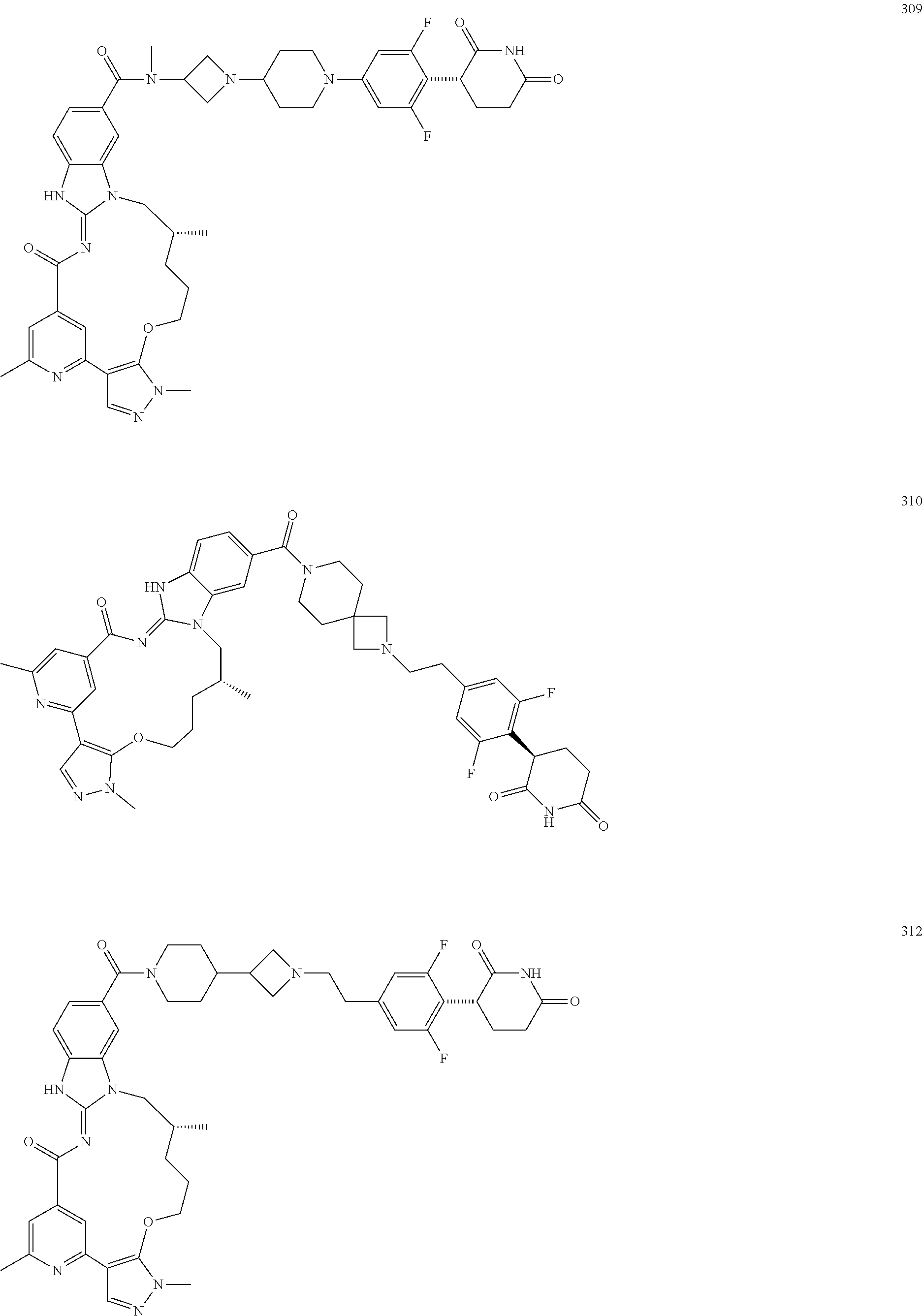

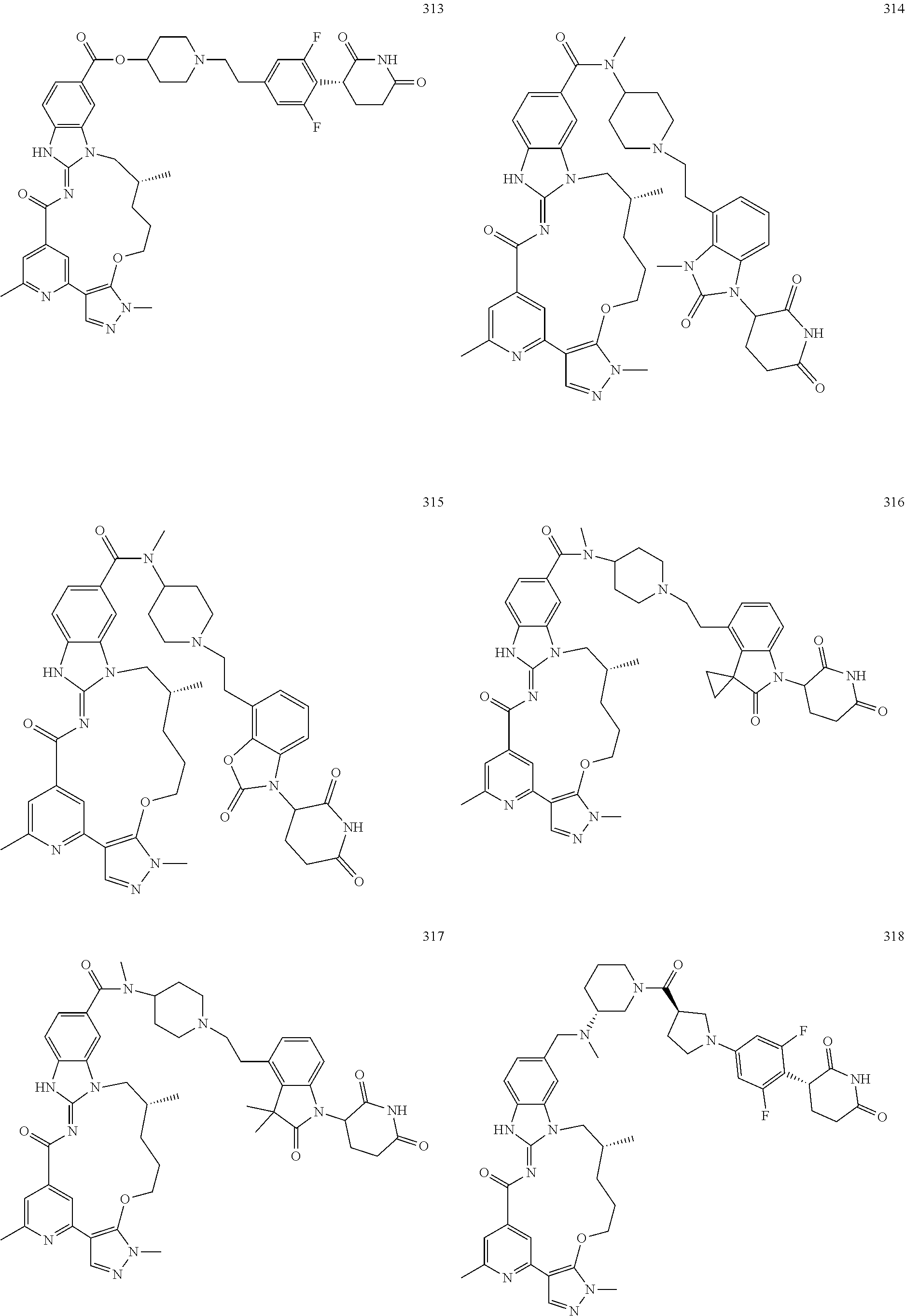

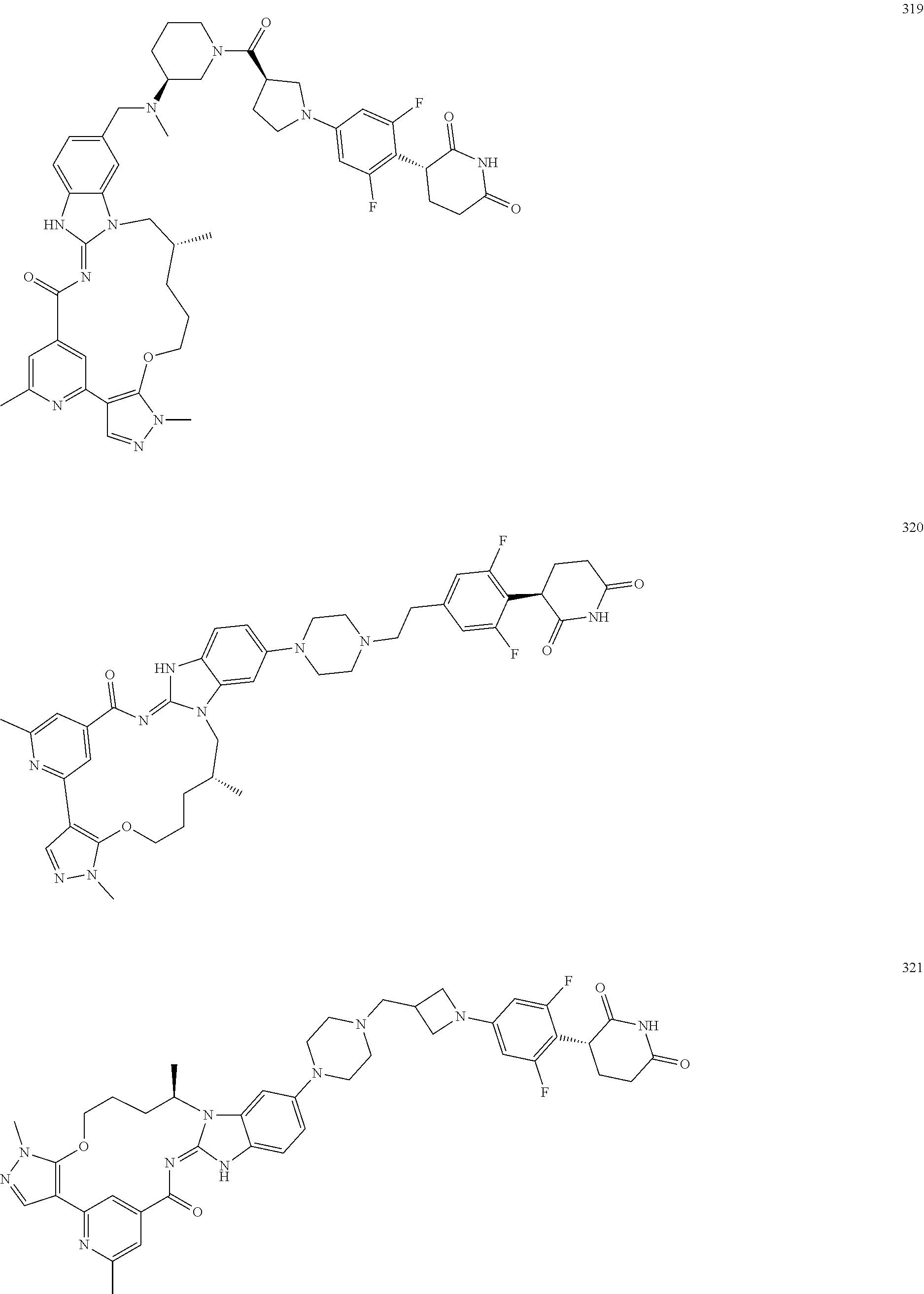 [SMILES: Cc1cc2cc(n1)-c1cnn(C)c1OCCC[C@@H](C)CN1/C(=N/C2=O)Nc2ccc(CN(C)[C@H]3CCCN(C(=O)[C@@H]4CCN(c5cc(F)c([C@H]6CCC(=O)NC6=O)c(F)c5)C4)C3)cc21 ; Cc1cc2cc(n1)-c1cnn(C)c1OCCC[C@@H](C)CN1/C(=N/C2=O)Nc2ccc(N3CCN(CCc4cc(F)c([C@H]5CCC(=O)NC5=O)c(F)c4)CC3)cc21 ; Cc1cc2cc(n1)-c1cnn(C)c1OCCC[C@@H](C)N1/C(=N/C2=O)Nc2ccc(N3CCN(CC4CN(c5cc(F)c([C@H]6CCC(=O)NC6=O)c(F)c5)C4)CC3)cc21]

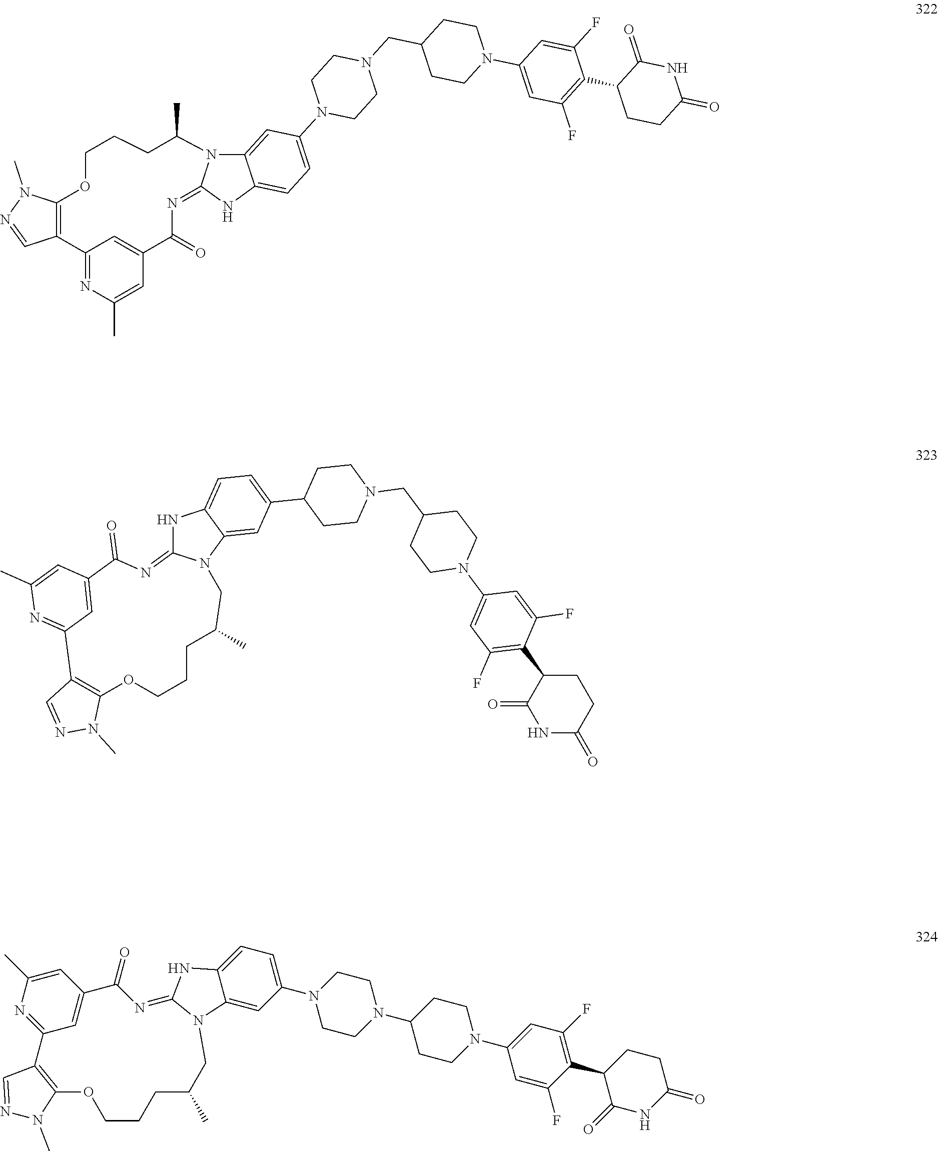 [SMILES: Cc1cc2cc(n1)-c1cnn(C)c1OCCC[C@@H](C)N1/C(=N/C2=O)Nc2ccc(N3CCN(CC4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(C3CCN(CC4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21]

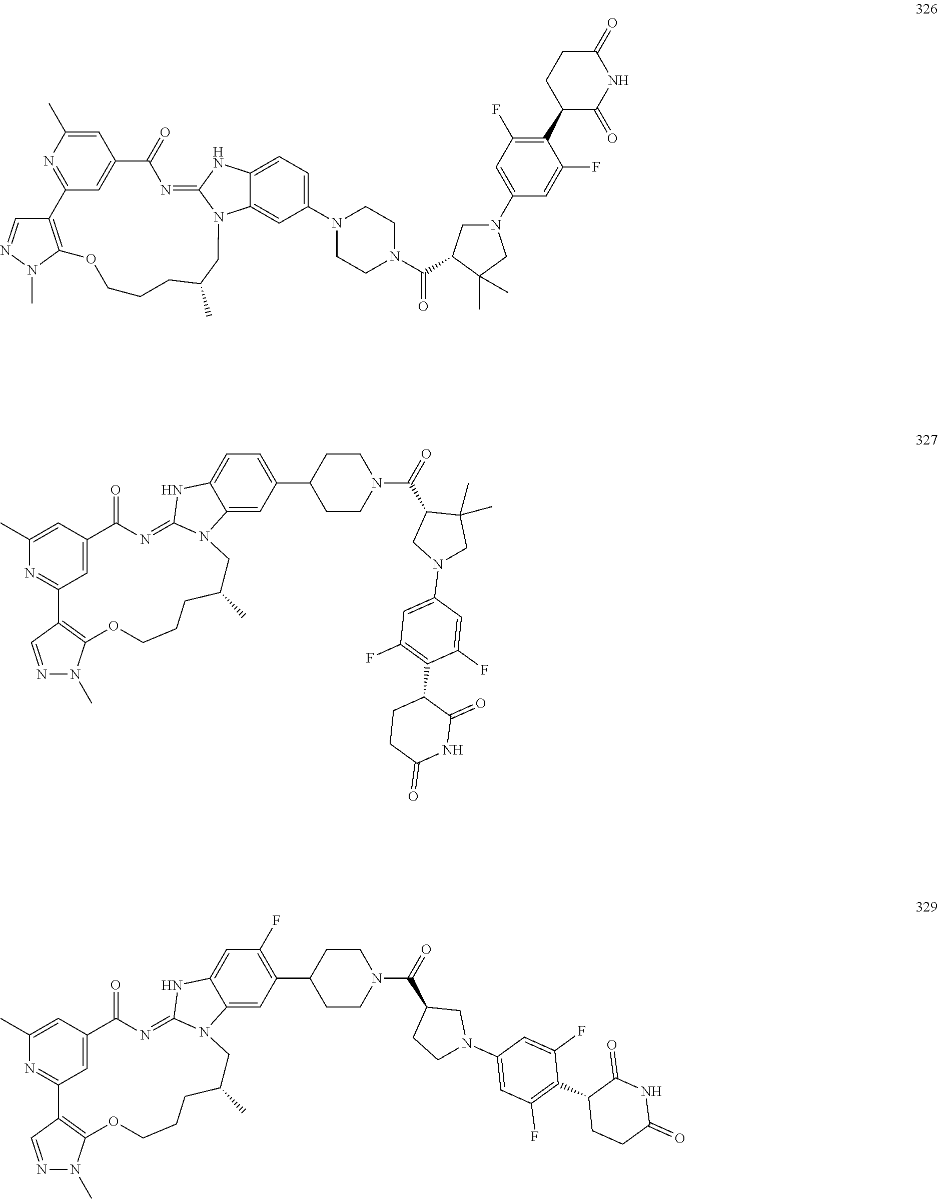

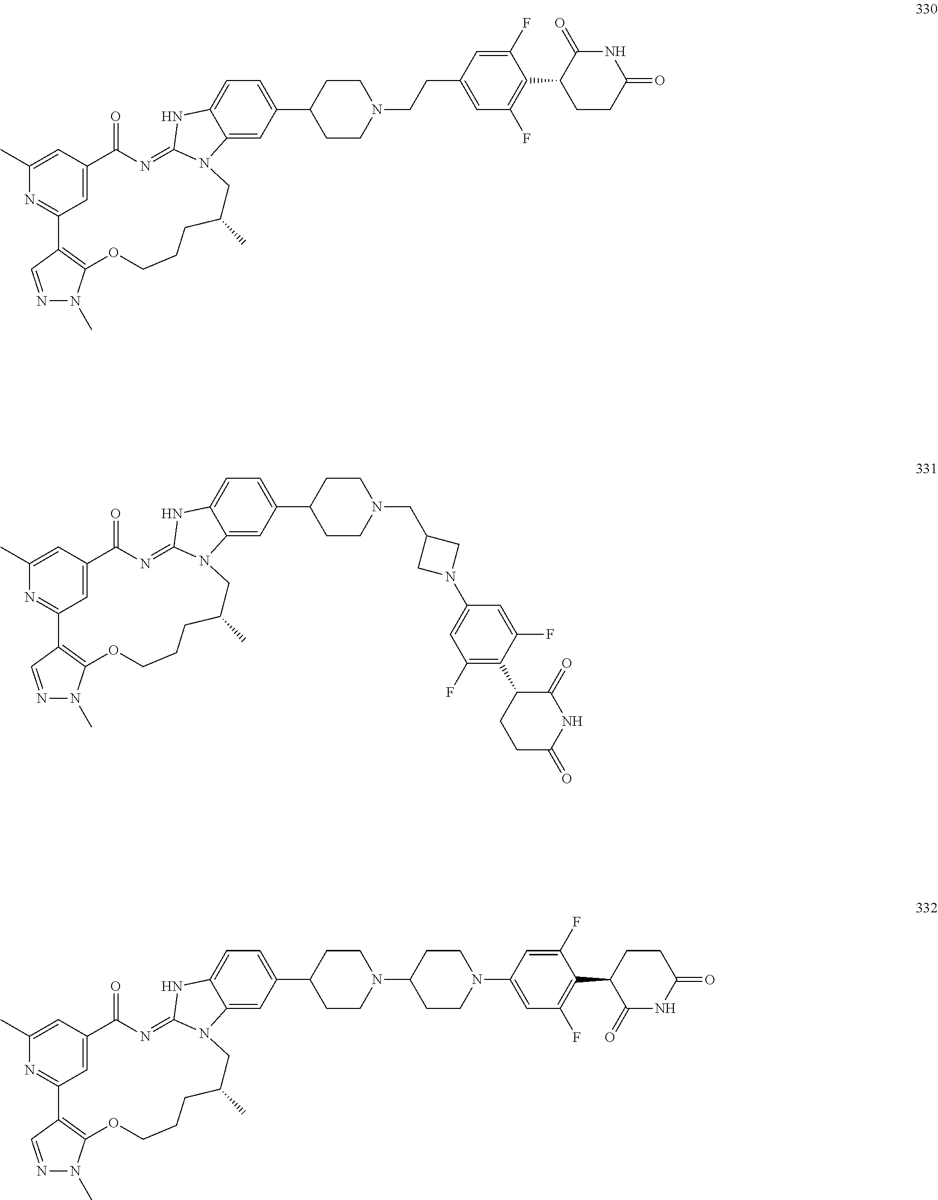 [SMILES: Cc1cc2cc(n1)-c1cnn(C)c1OCCC[C@@H](C)CN1/C(=N/C2=O)Nc2ccc(C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C3CCN(CC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C3CCN(C4CCN(c5cc(F)c([C@H]6CCC(=O)NC6=O)c(F)c5)CC4)CC3)cc21]

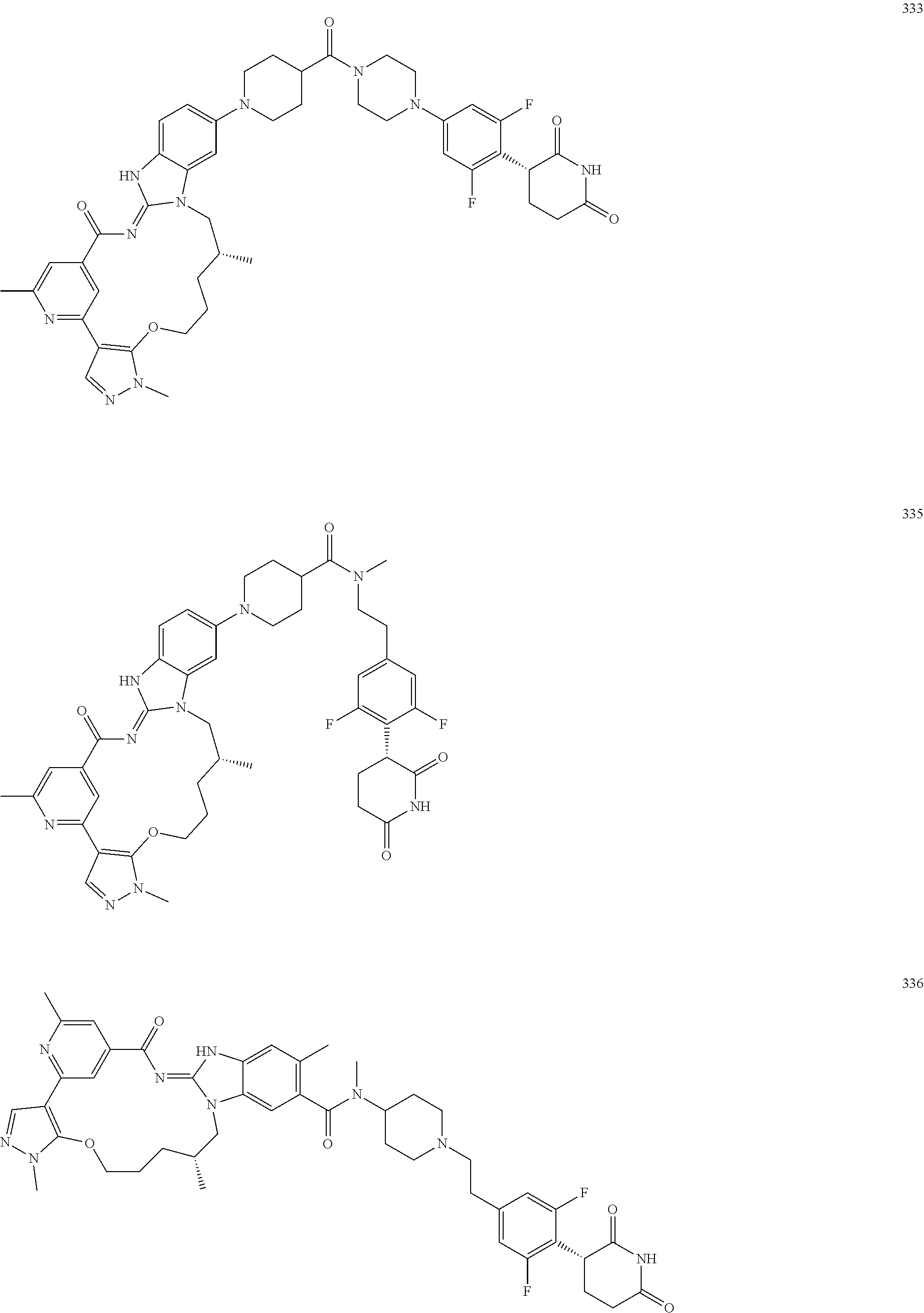 [SMILES: Cc1cc2cc(n1)-c1cnn(C)c1OCCC[C@@H](C)CN1/C(=N/C2=O)Nc2ccc(N3CCC(C(=O)N4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCC(C(=O)N(C)CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2cc(C)c(C(=O)N(C)C3CCN(CCc4cc(F)c([C@H]5CCC(=O)NC5=O)c(F)c4)CC3)cc21]

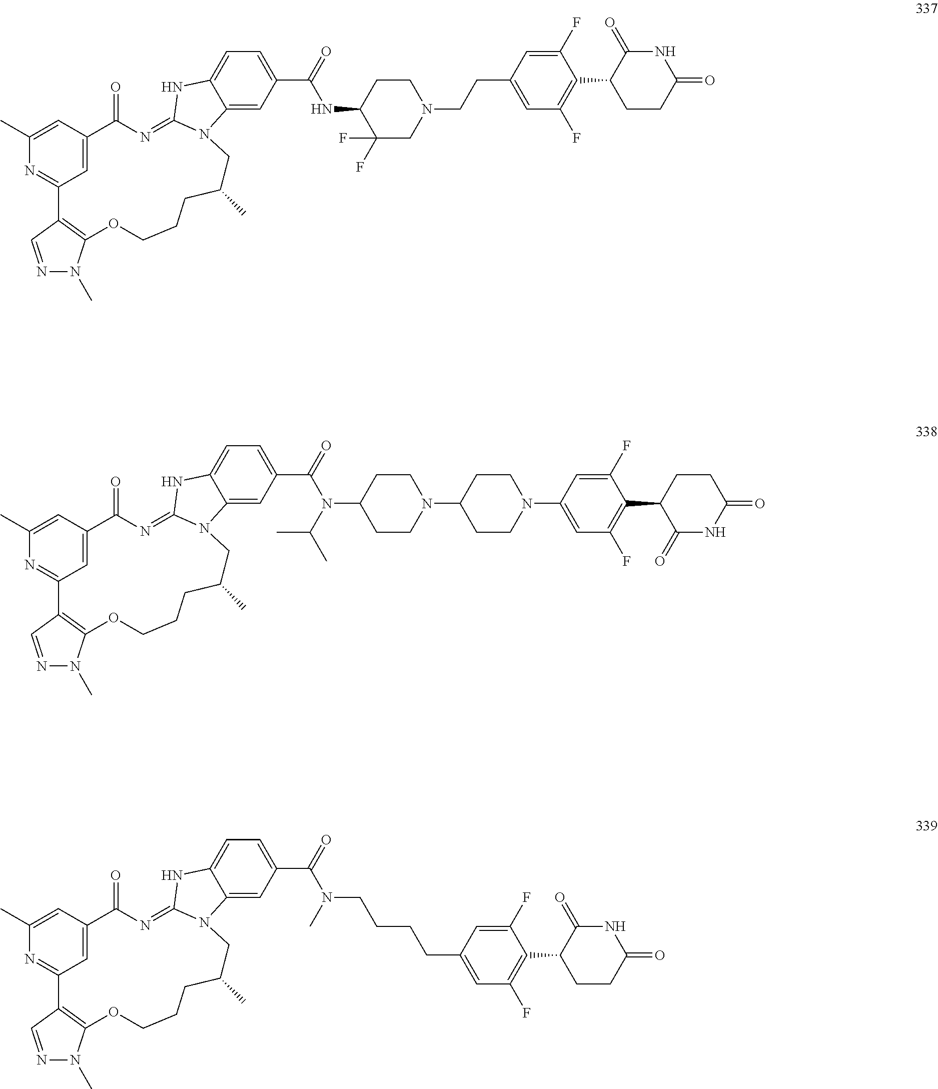 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)CC3(F)F)cc21 ; Cc1cc2cc(n1)-c1cnn(C)c1OCCC[C@@H](C)CN1/C(=N/C2=O)Nc2ccc(C(=O)N(C(C)C)C3CCN(C4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(C(=O)N(C)CCCCc3cc(F)c([C@H]4CCC(=O)NC4=O)c(F)c3)cc21]

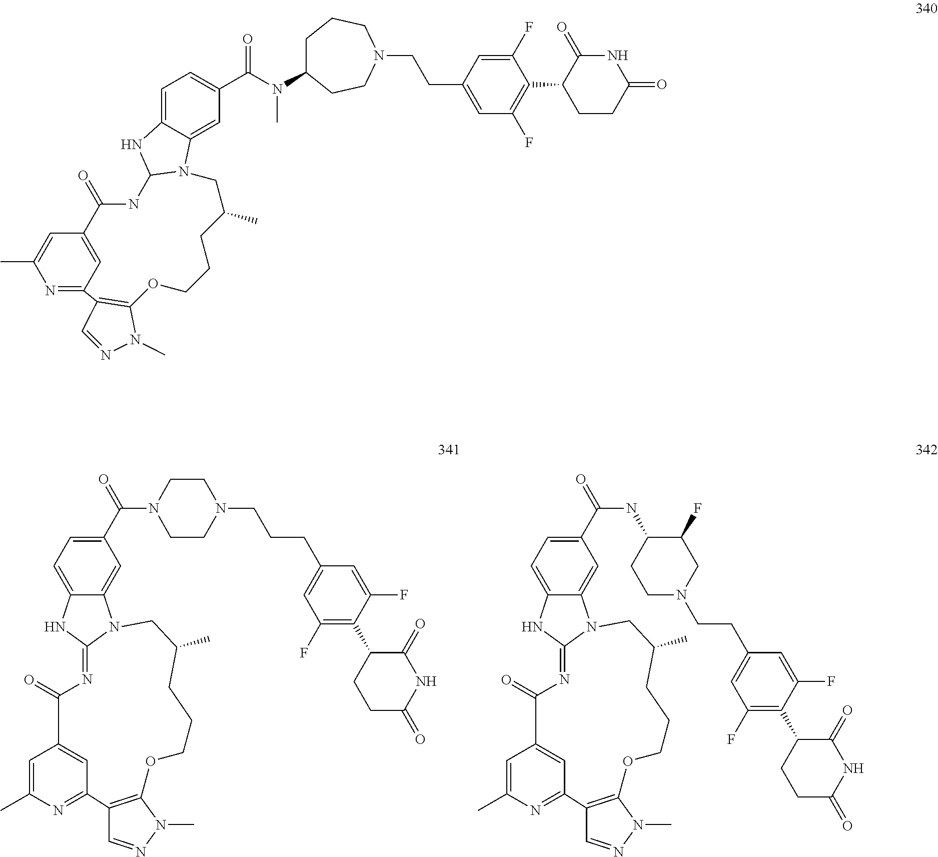 [SMILES: Cc1cc2cc(n1)-c1cnn(C)c1OCCC[C@@H](C)CN1c3cc(C(=O)N(C)[C@H]4CCCN(CCc5cc(F)c([C@H]6CCC(=O)NC6=O)c(F)c5)CC4)ccc3NC1NC2=O ; Cc1cc2cc(n1)-c1cnn(C)c1OCCC[C@@H](C)CN1/C(=N/C2=O)Nc2ccc(C(=O)N3CCN(CCCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)C[C@@H]3F)cc21]

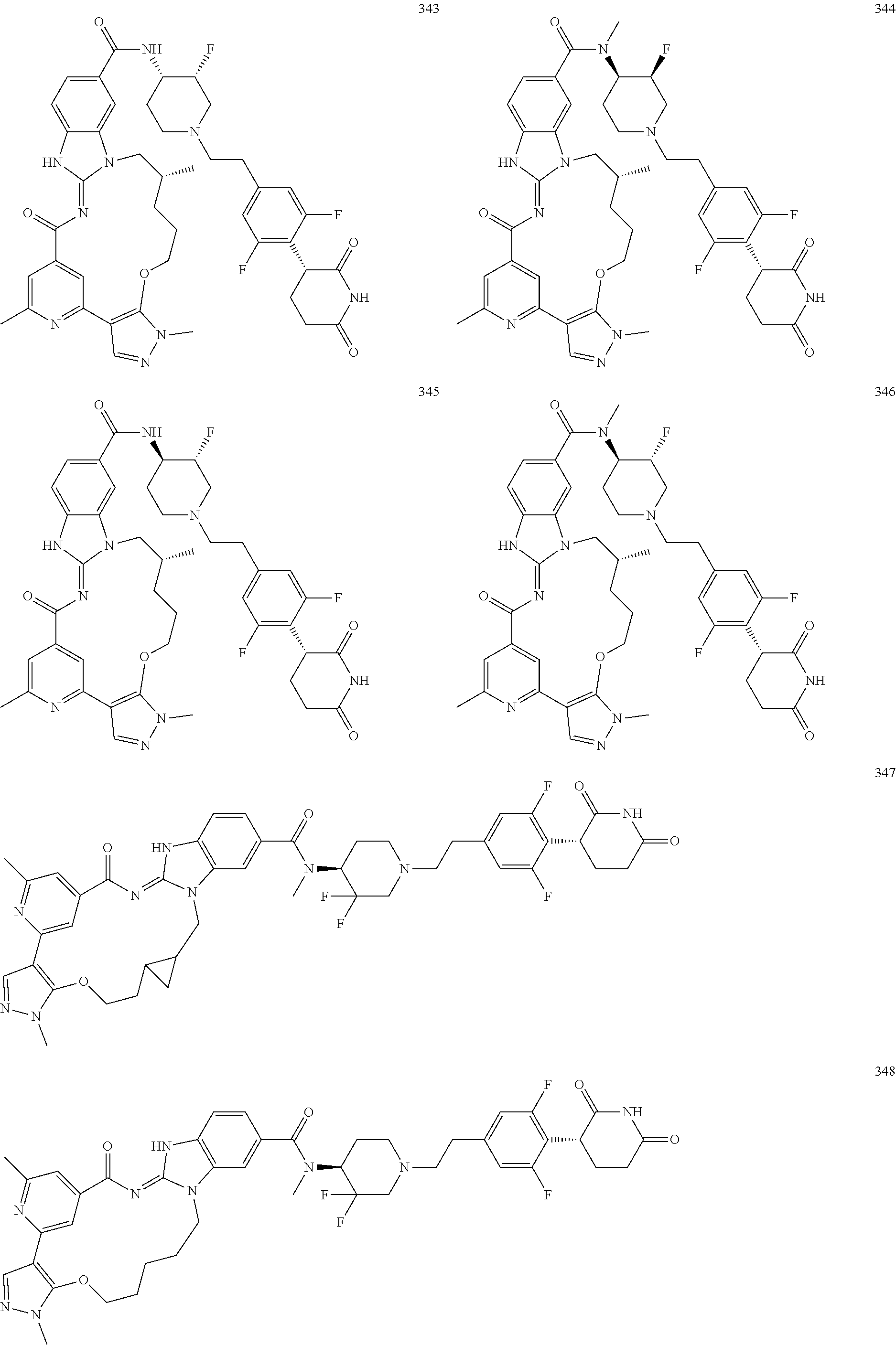

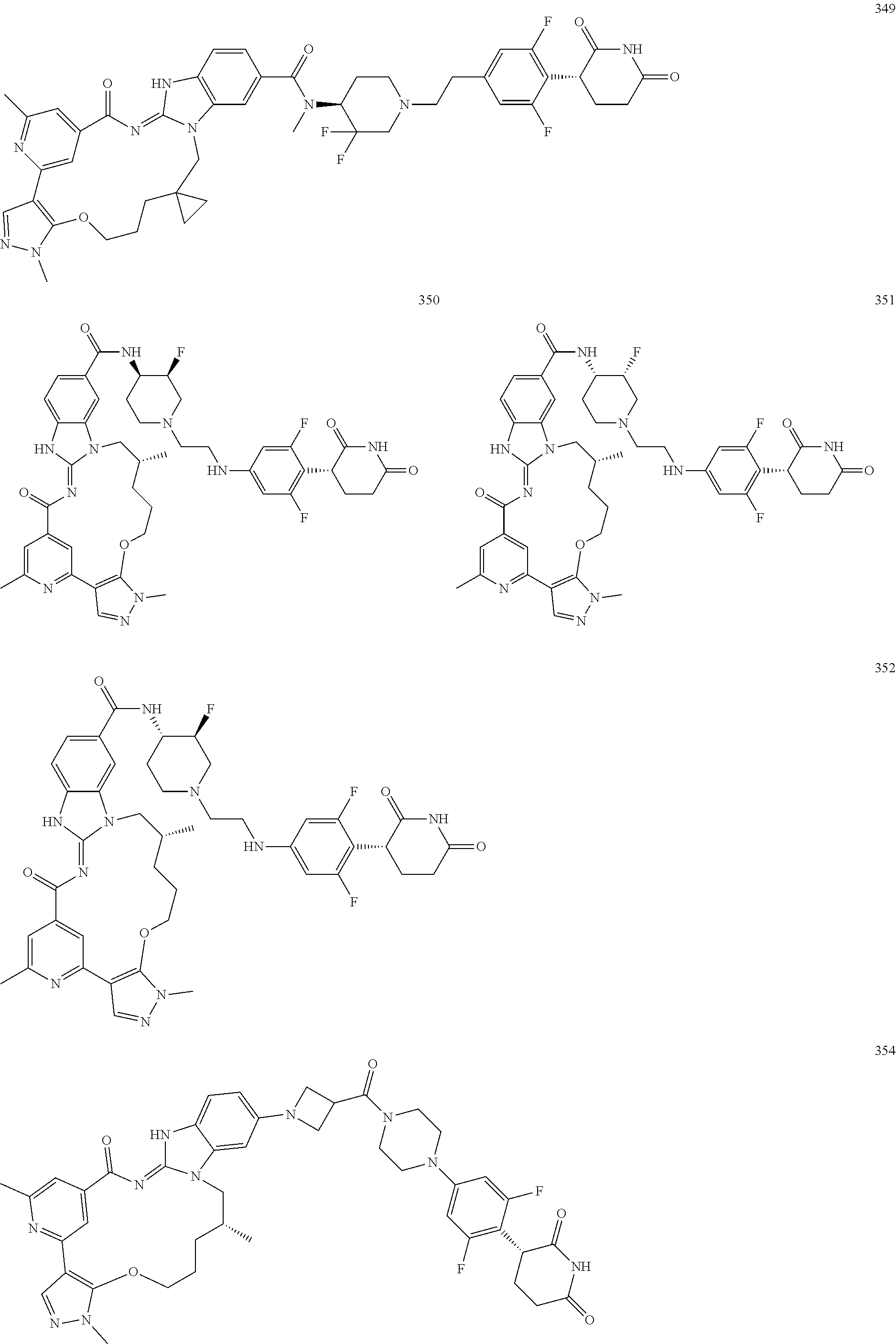

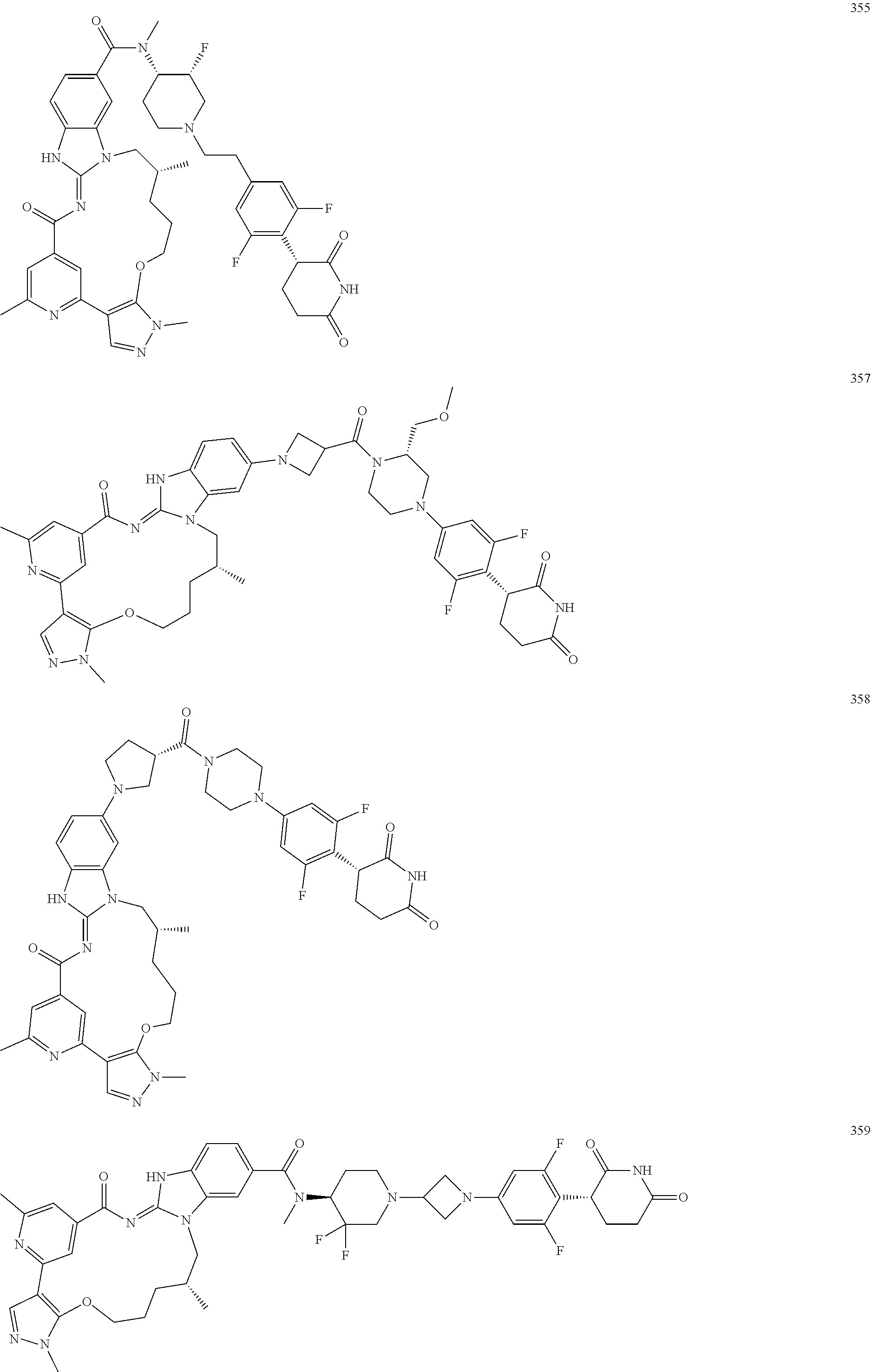

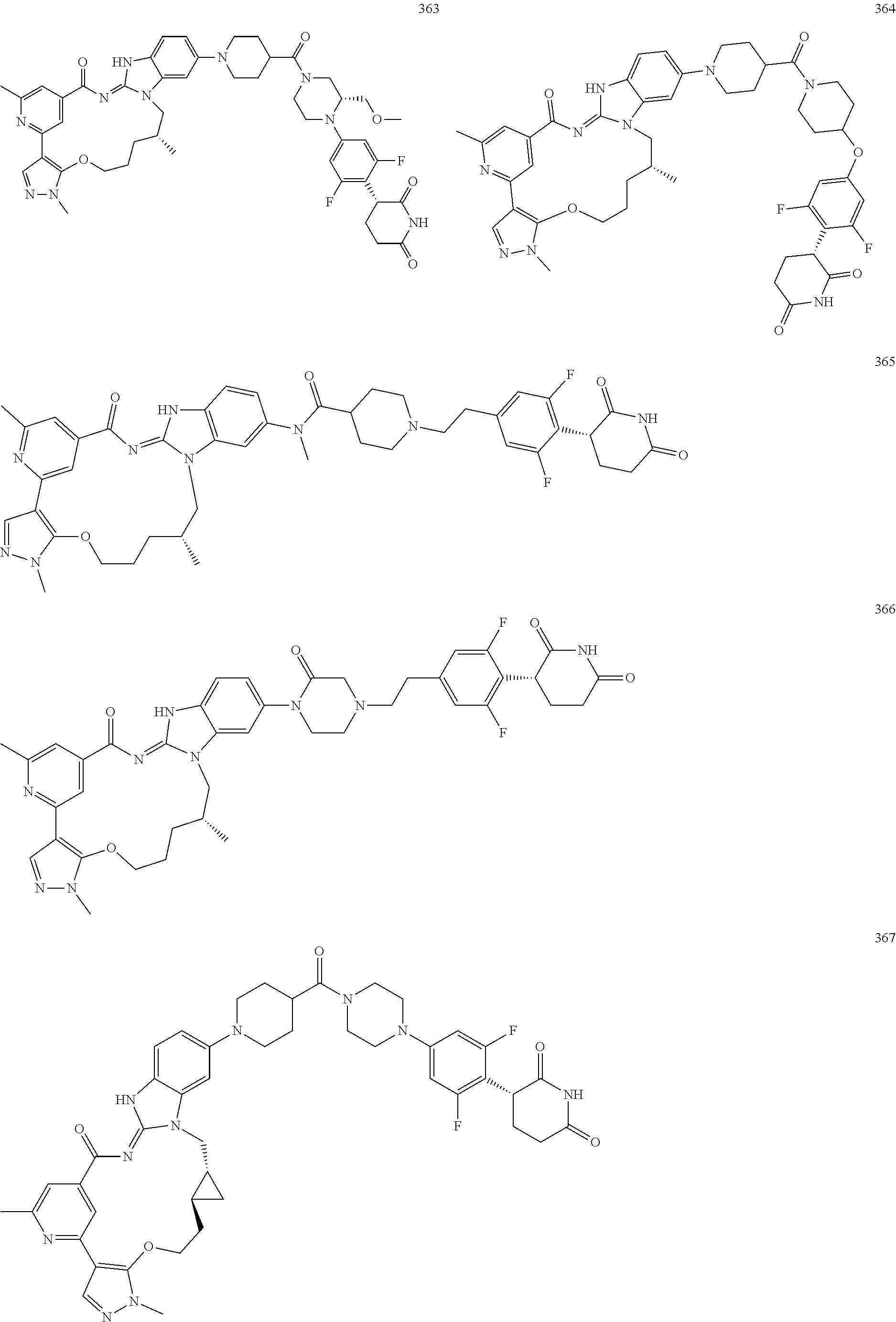

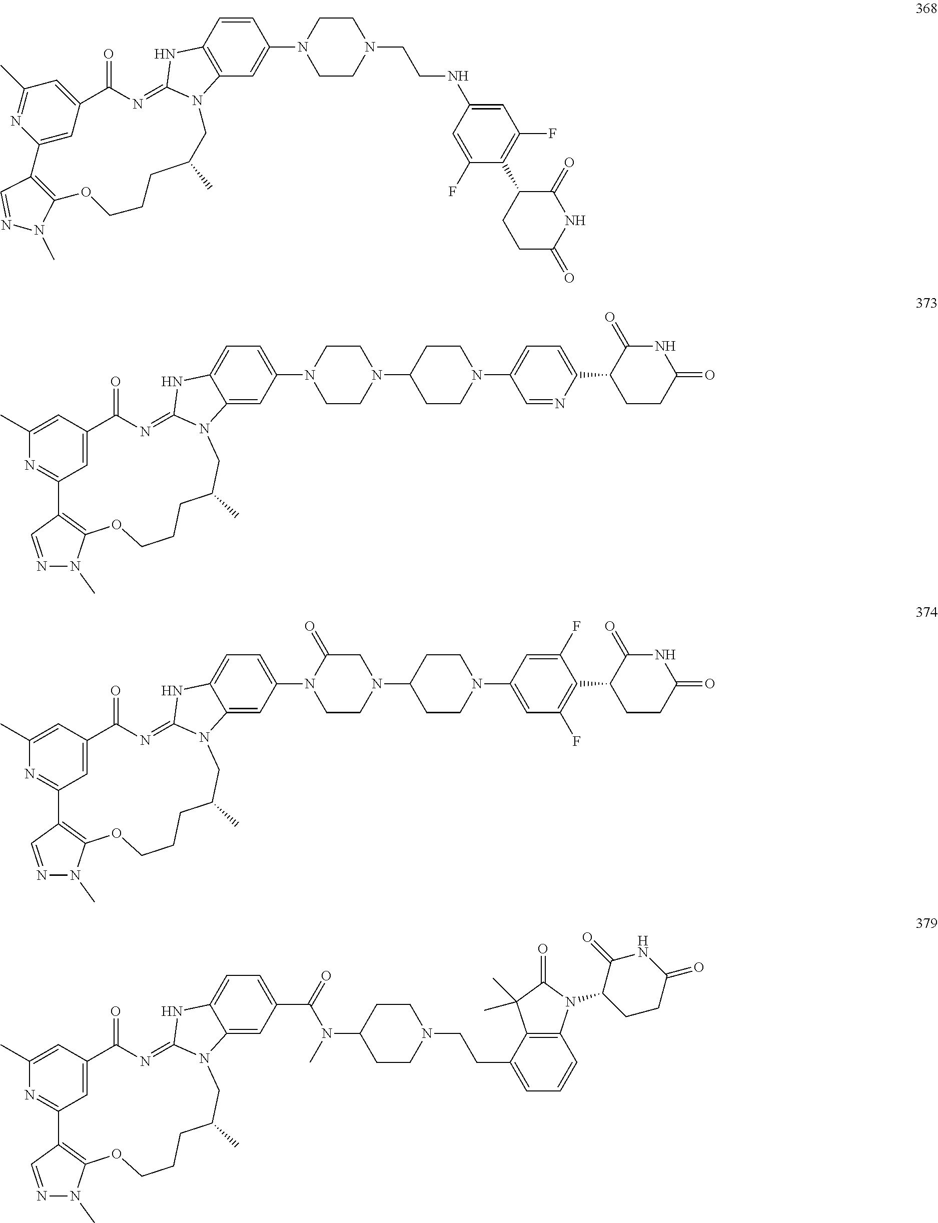 [SMILES: Cc1cc2cc(n1)-c1cnn(C)c1OCCC[C@@H](C)CN1/C(=N/C2=O)Nc2ccc(N3CCN(CCNc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(N3CCN(C4CCN(c5ccc([C@H]6CCC(=O)NC6=O)nc5)CC4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3=O)cc21 ; Cc1cc2cc(n1)-c1cnn(C)c1OCCC[C@@H](C)CN1/C(=N/C2=O)Nc2ccc(C(=O)N(C)C3CCN(CCc4cccc5c4C(C)(C)C(=O)N5[C@H]4CCC(=O)NC4=O)CC3)cc21]

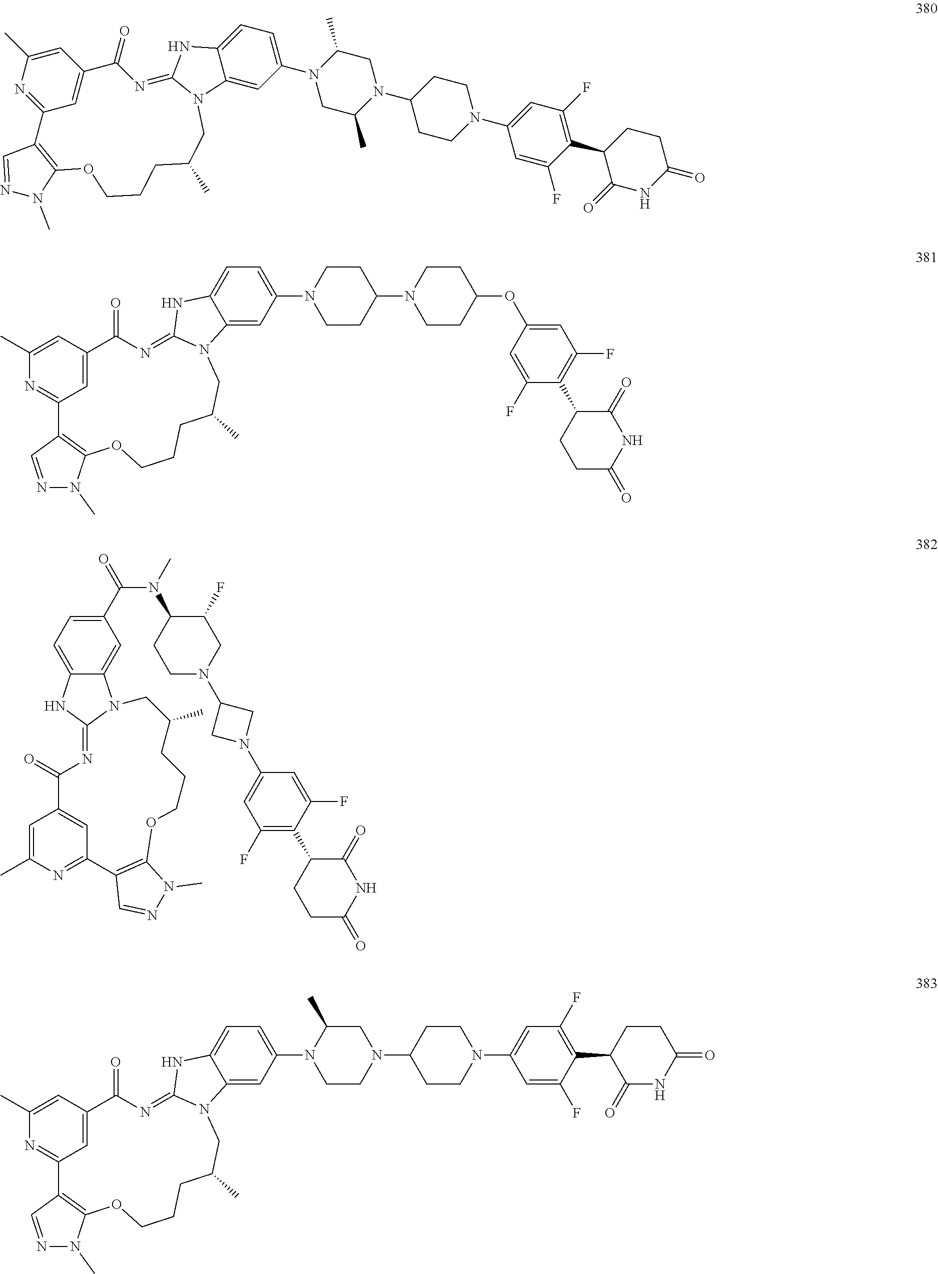

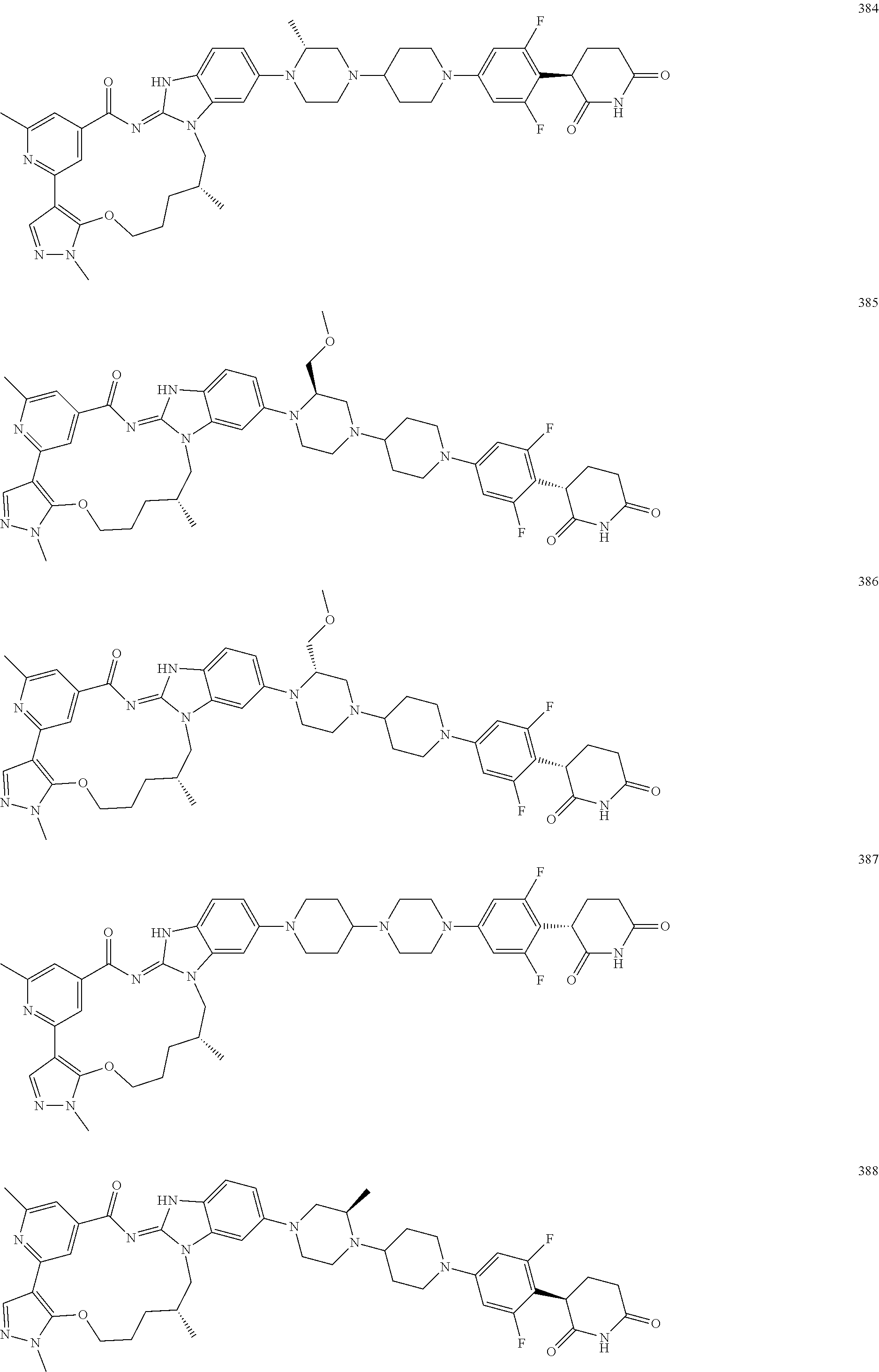

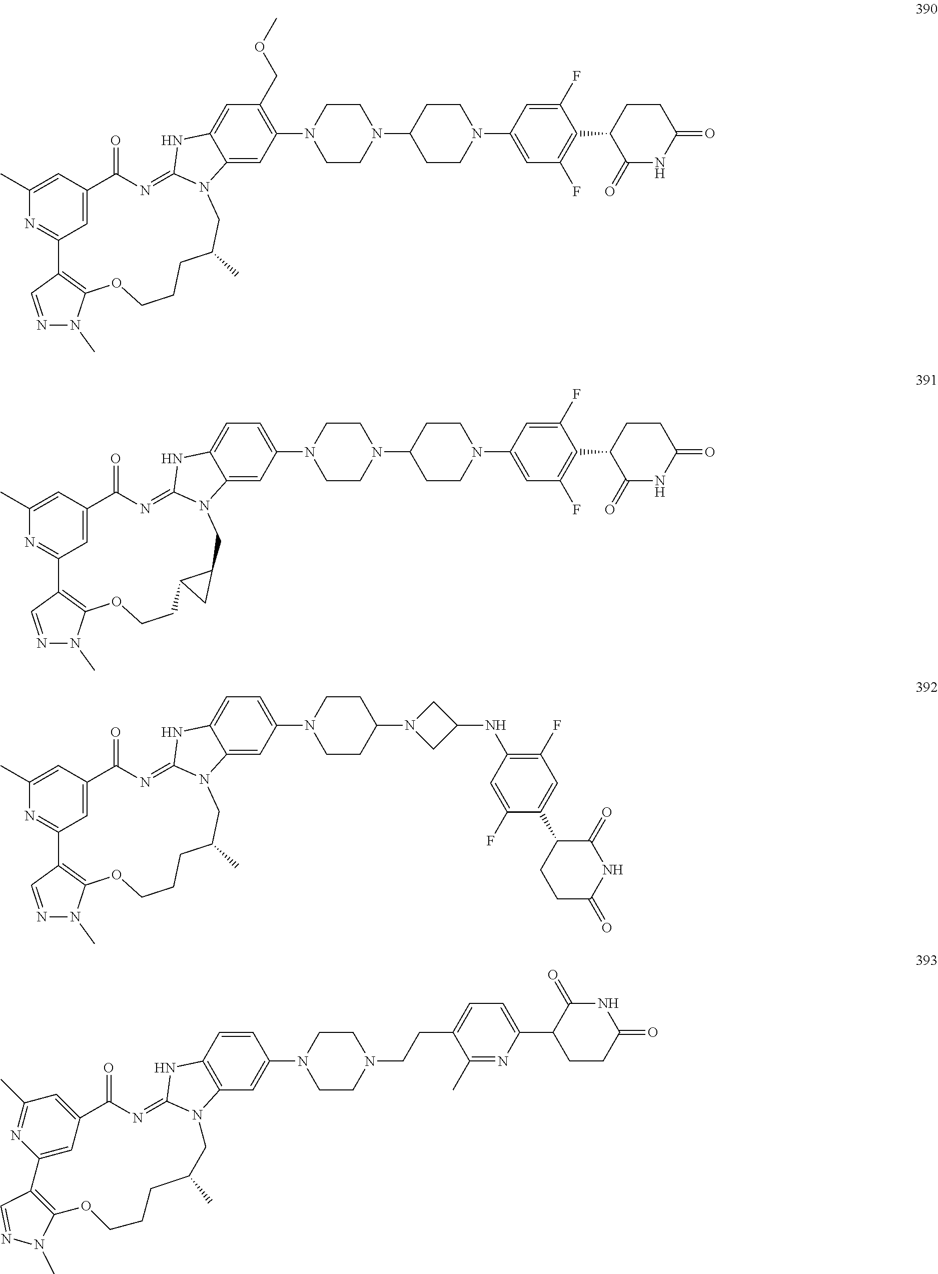 [SMILES: COCc1cc2c(cc1N1CCN(C3CCN(c4cc(F)c([C@@H]5CCC(=O)NC5=O)c(F)c4)CC3)CC1)N1C[C@H](C)CCCOc3c(cnn3C)-c3cc(cc(C)n3)C(=O)/N=C/1N2 ; Cc1cc2cc(n1)-c1cnn(C)c1OCC[C@H]1C[C@@H]1CN1/C(=N/C2=O)Nc2ccc(N3CCN(C4CCN(c5cc(F)c([C@@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCC(N4CC(Nc5cc(F)c([C@H]6CCC(=O)NC6=O)cc5F)C4)CC3)cc21 ; Cc1cc2cc(n1)-c1cnn(C)c1OCCC[C@@H](C)CN1/C(=N/C2=O)Nc2ccc(N3CCN(CCc4ccc(C5CCC(=O)NC5=O)nc4C)CC3)cc21]

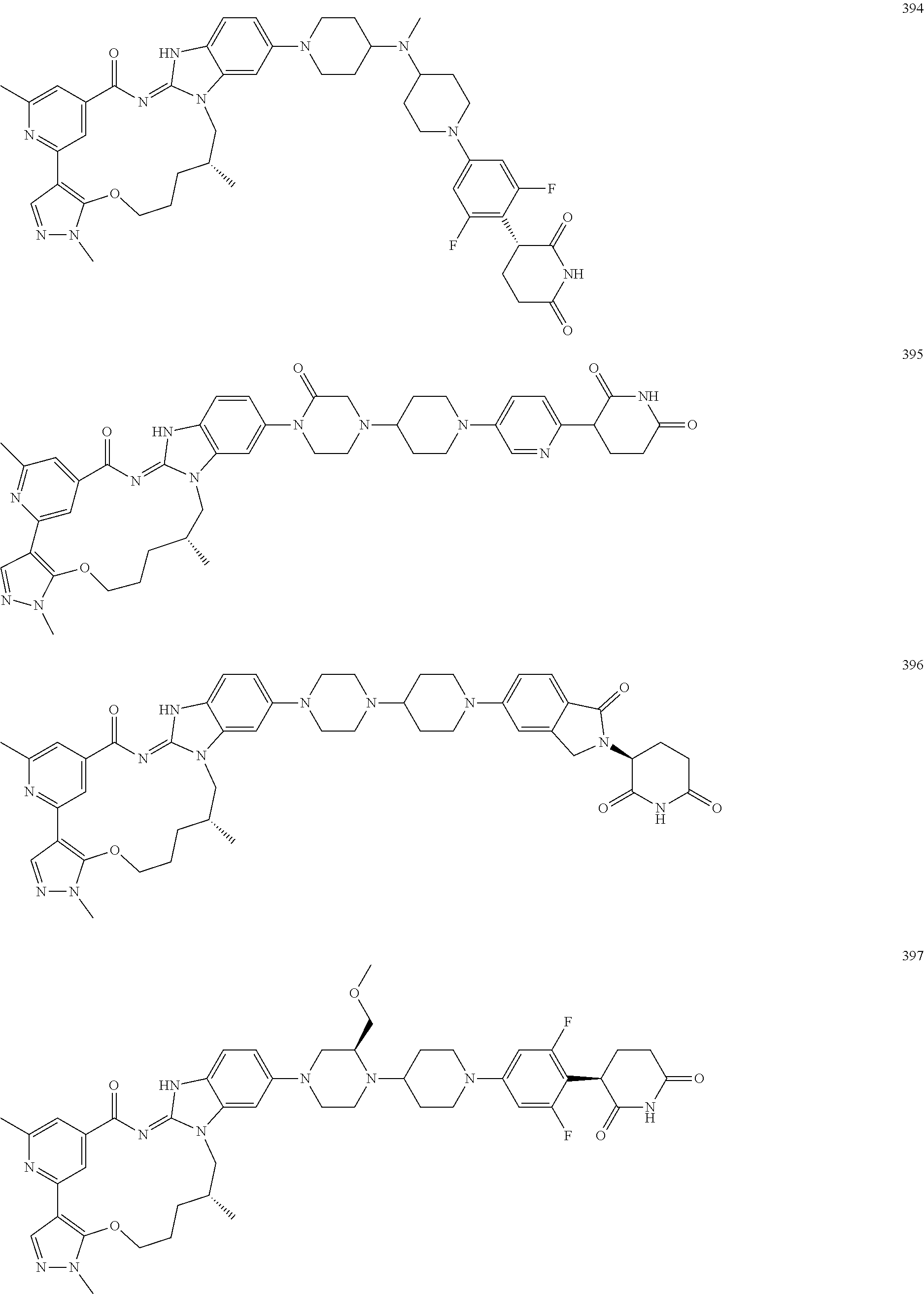

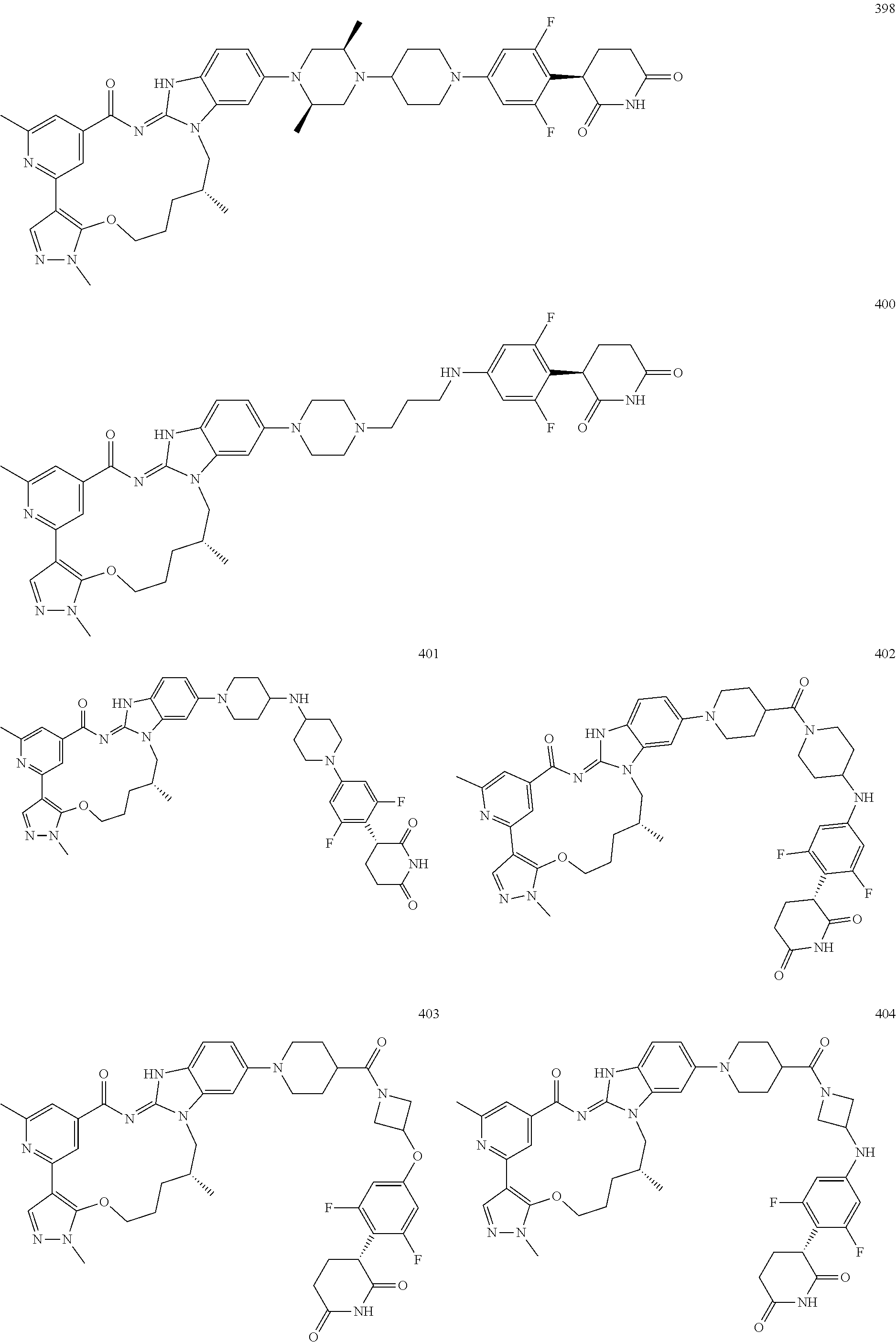

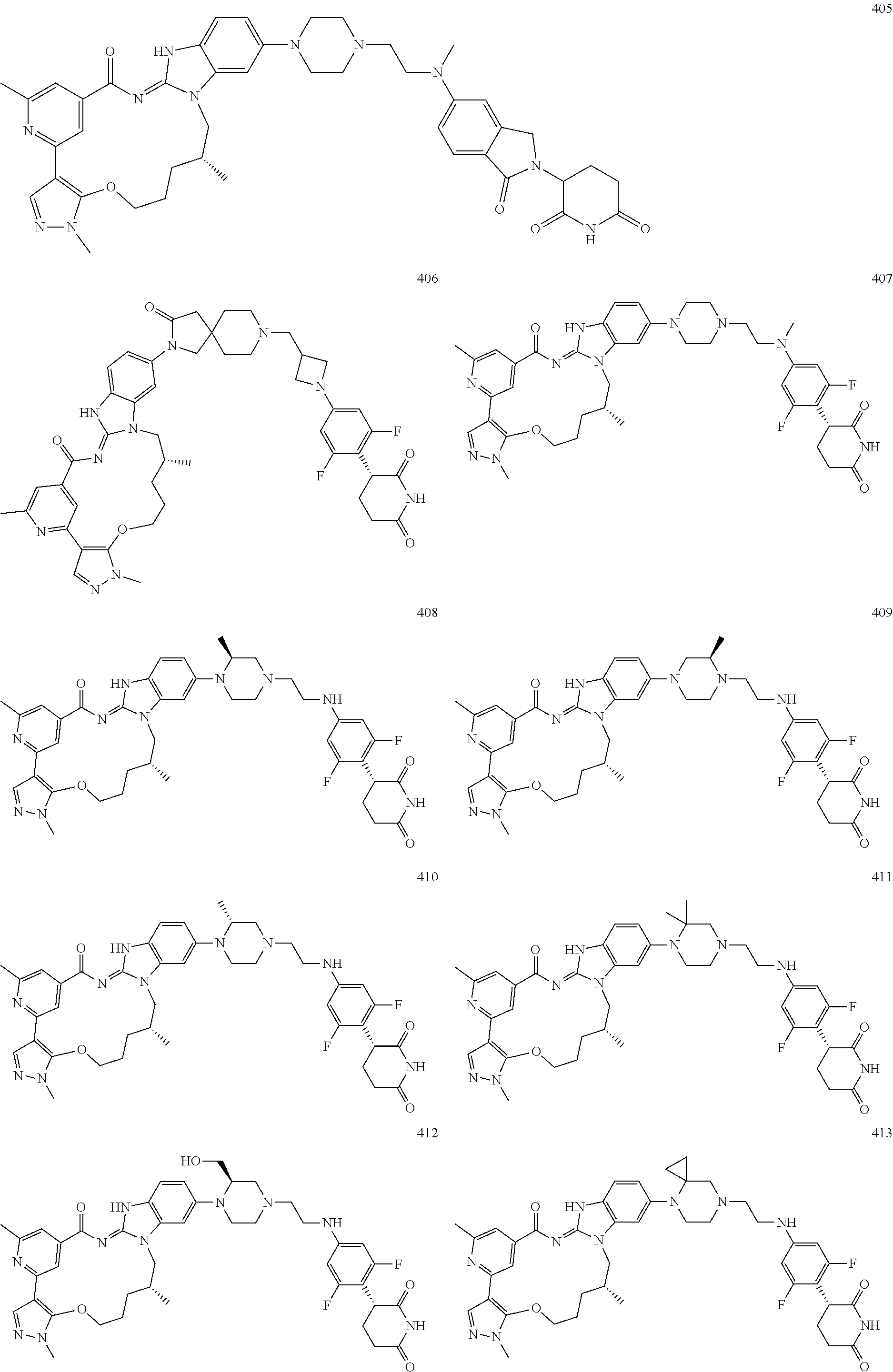

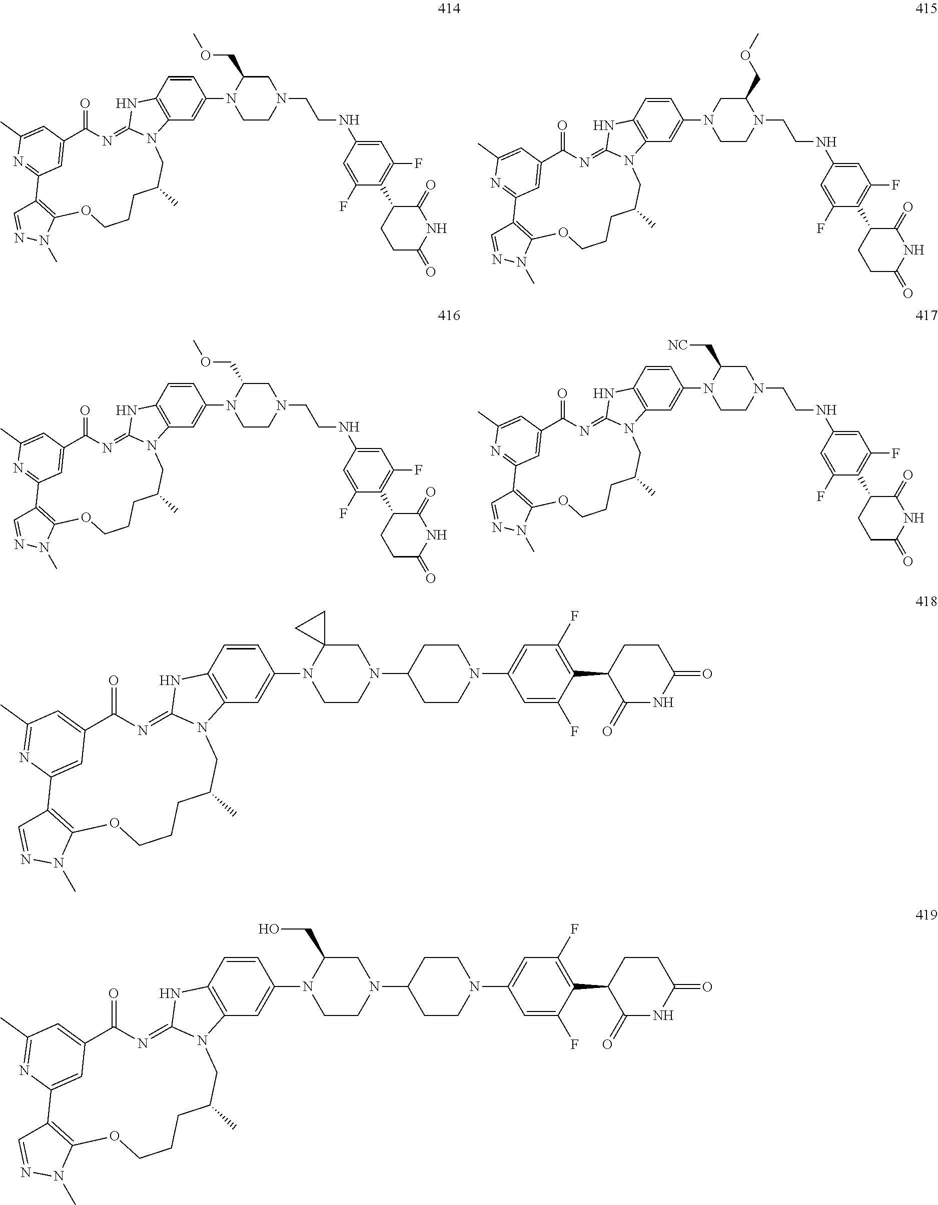

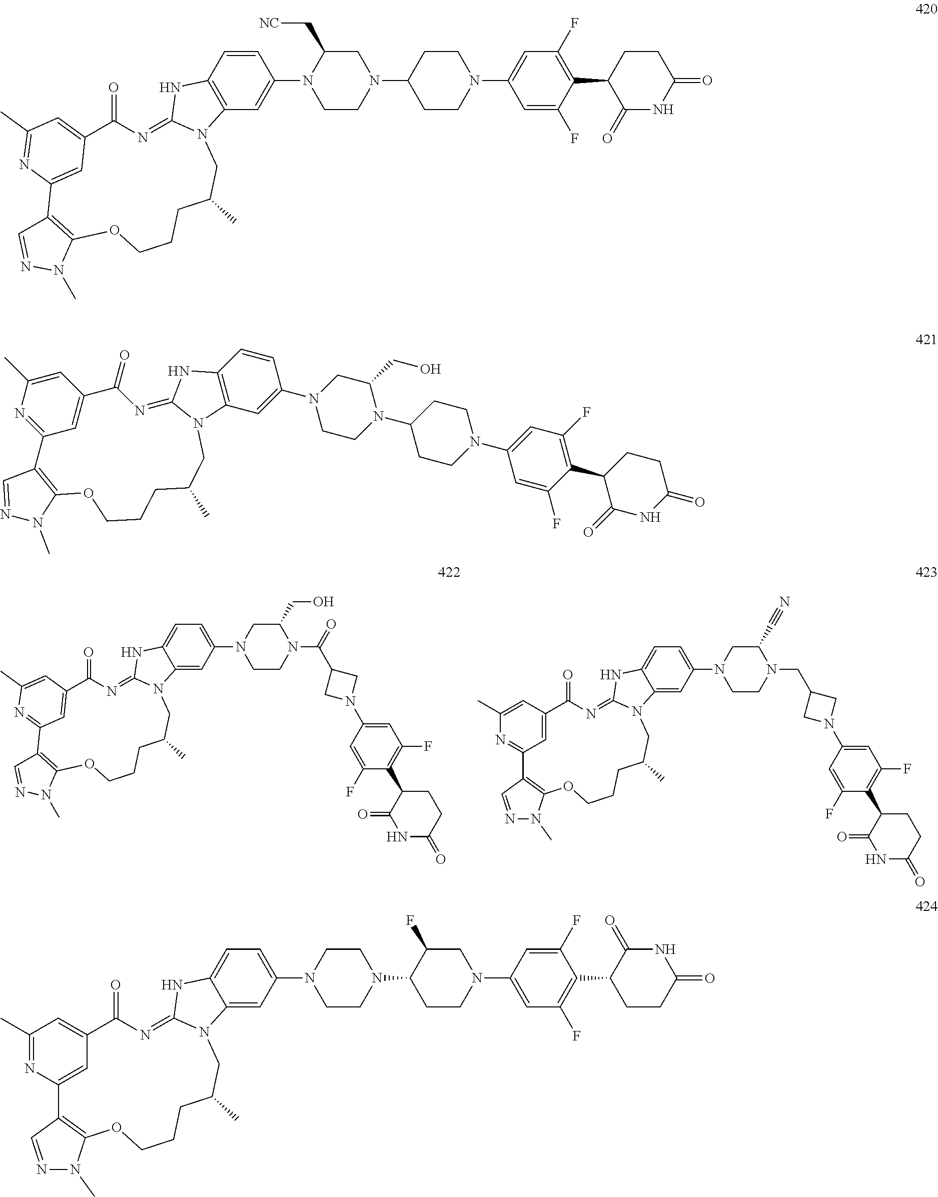

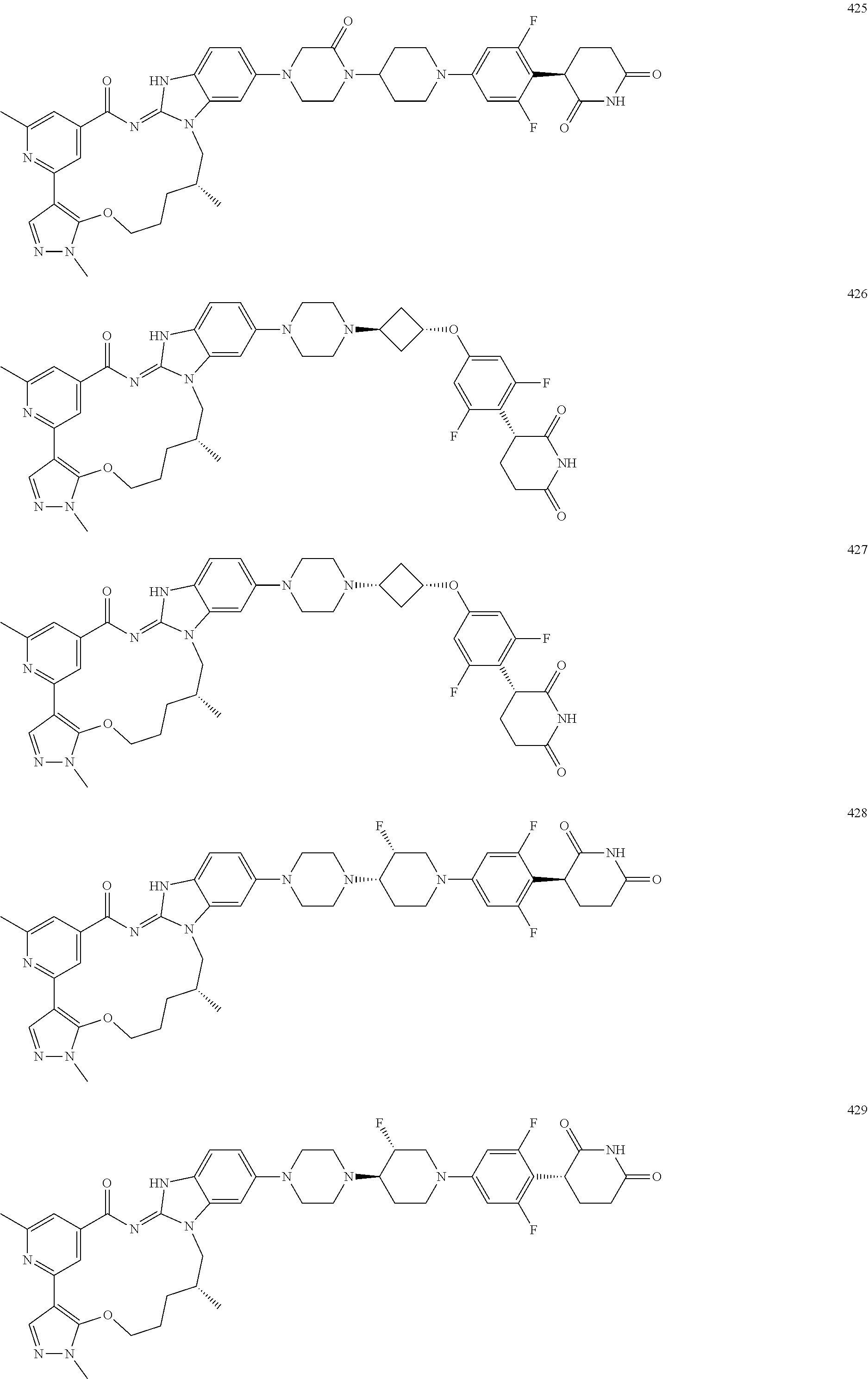

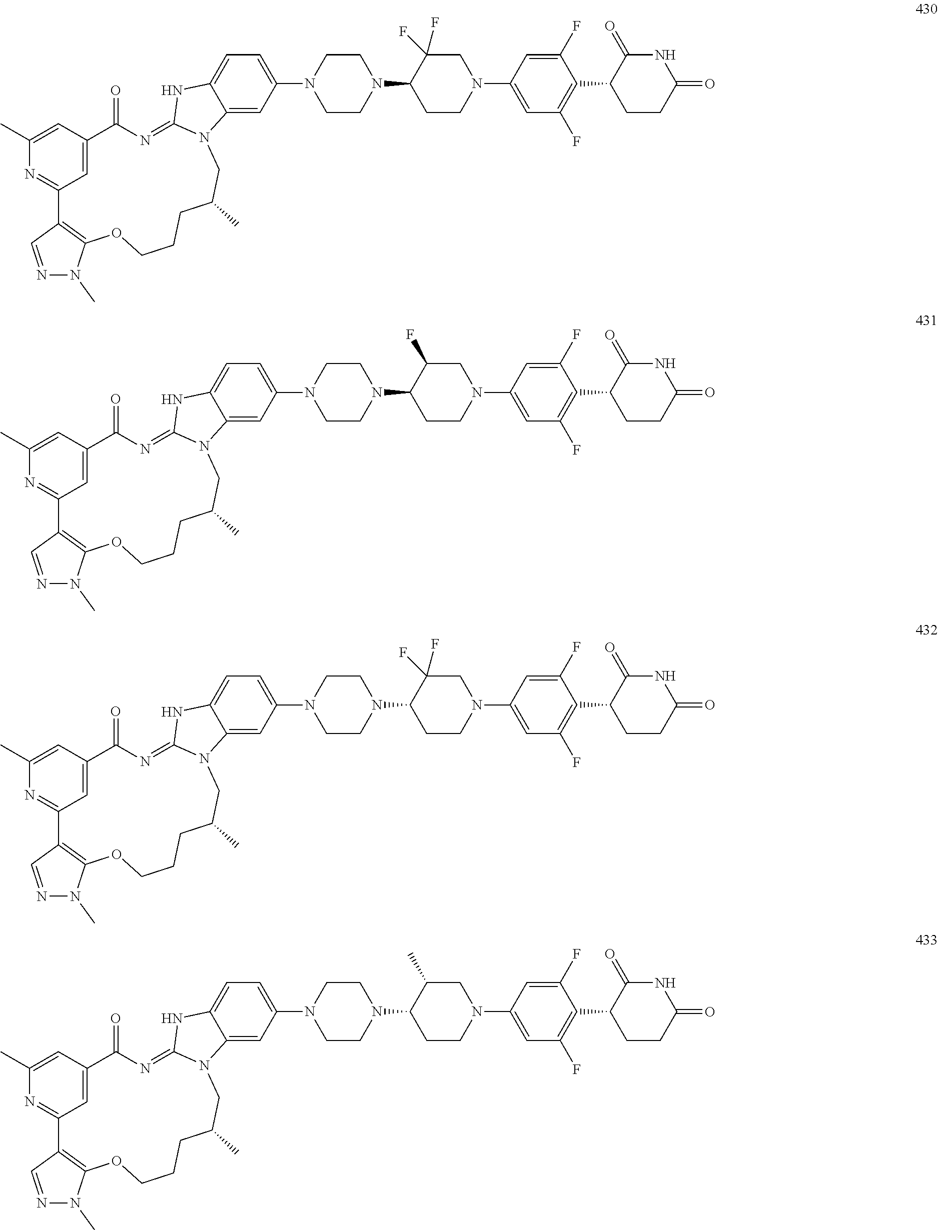

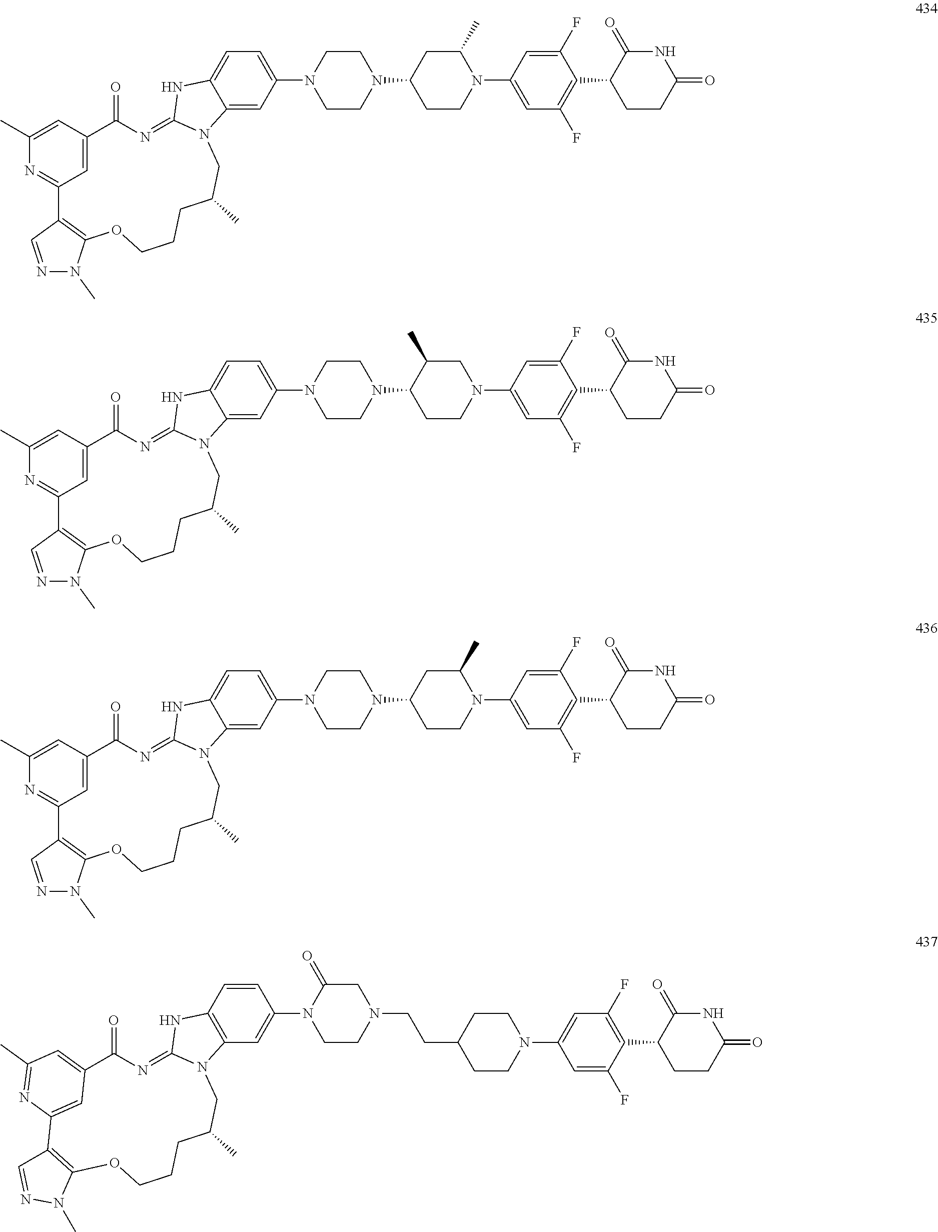

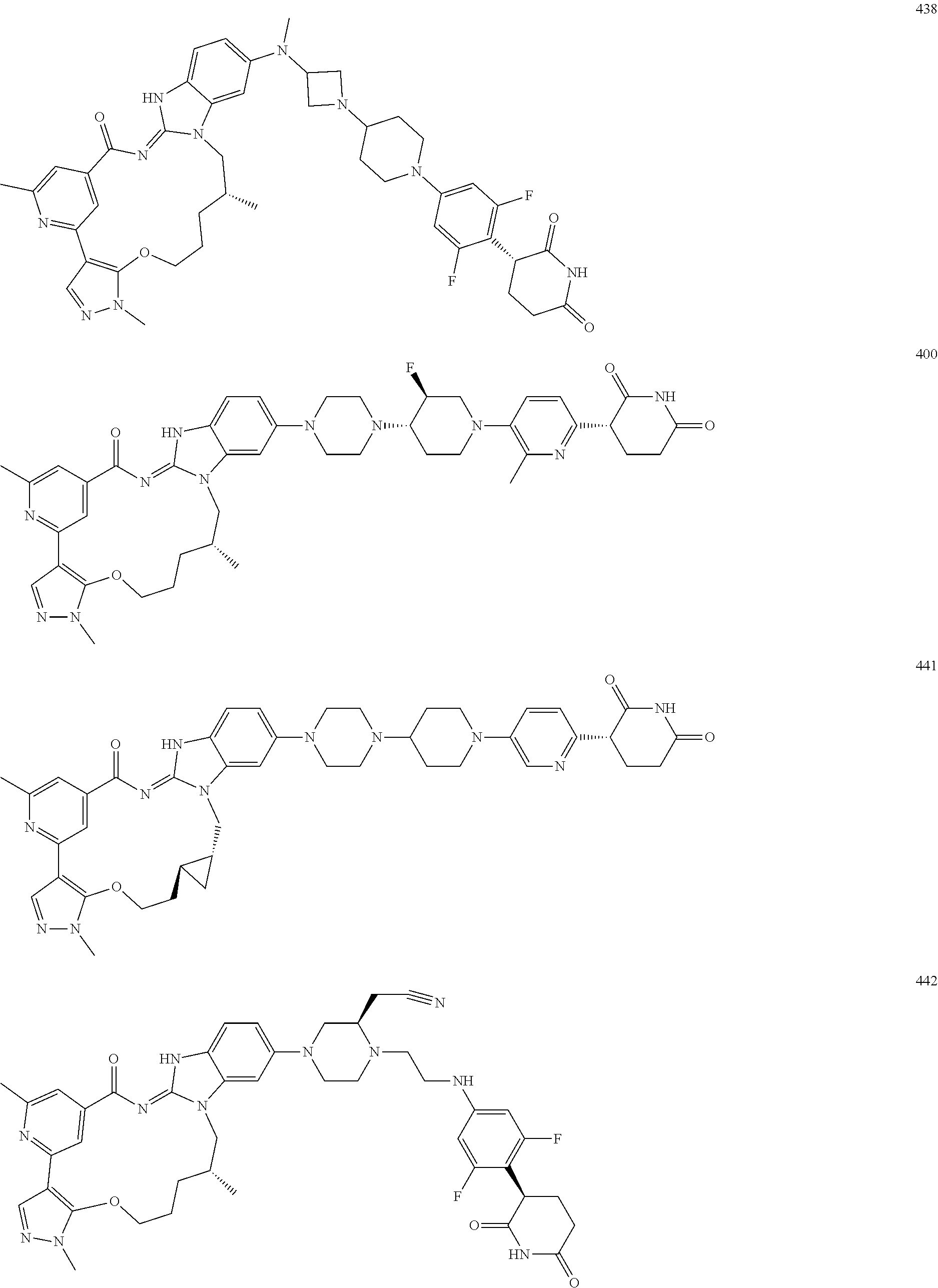

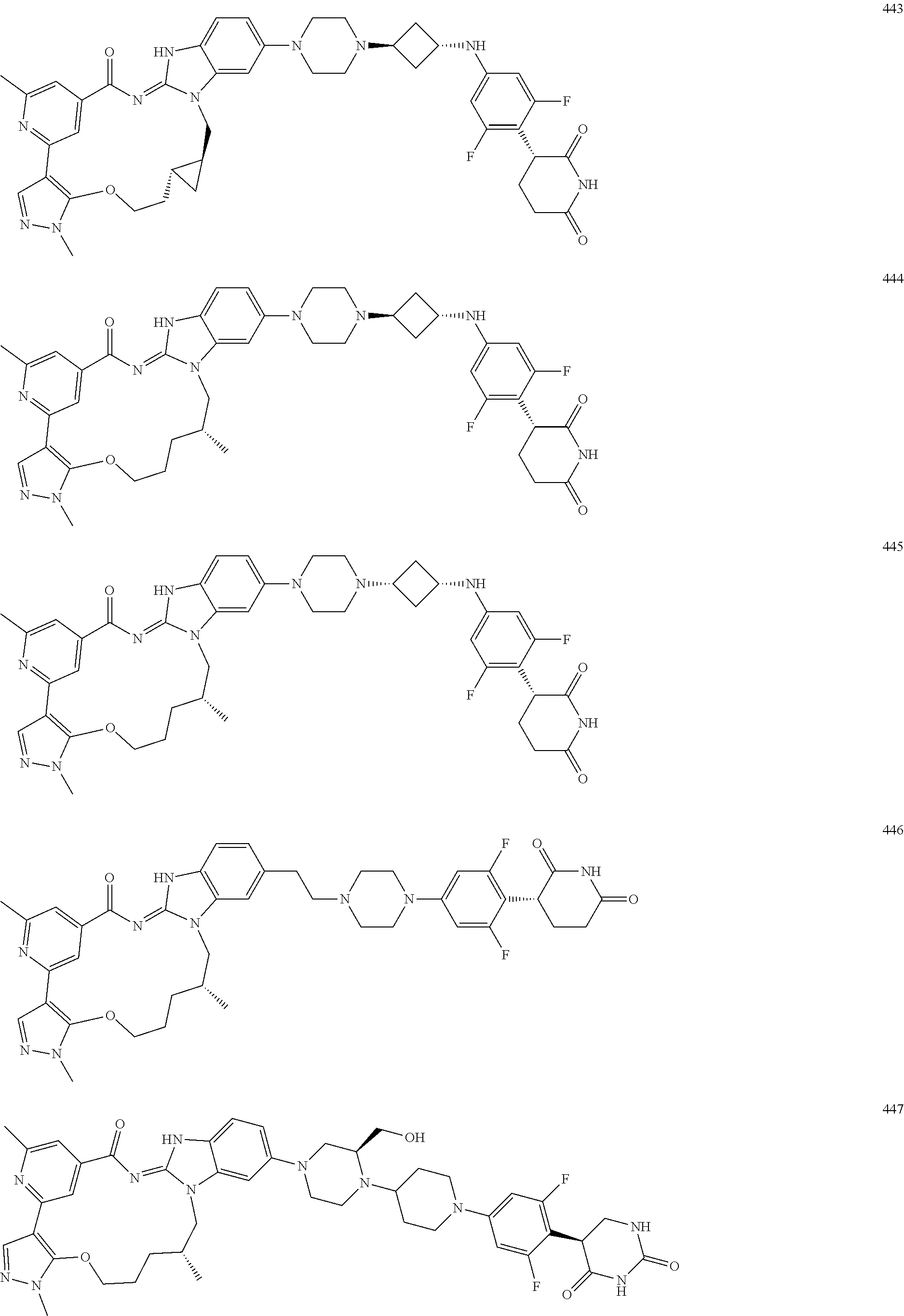

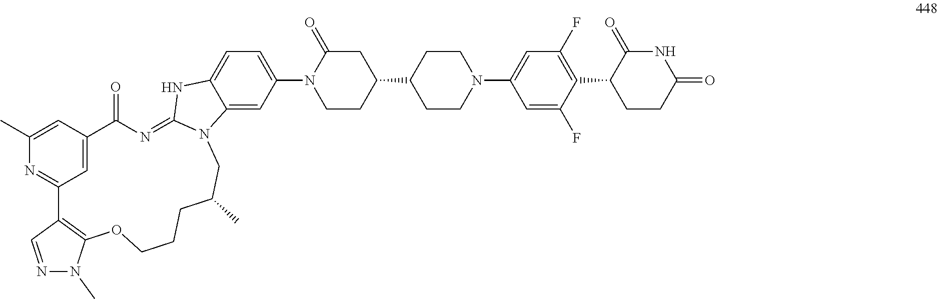 [SMILES: Cc1cc2cc(n1)-c1cnn(C)c1OCCC[C@@H](C)CN1/C(=N/C2=O)Nc2ccc(N3CC[C@@H](C4CCN(c5cc(F)c([C@H]6CCC(=O)NC6=O)c(F)c5)CC4)CC3=O)cc21]

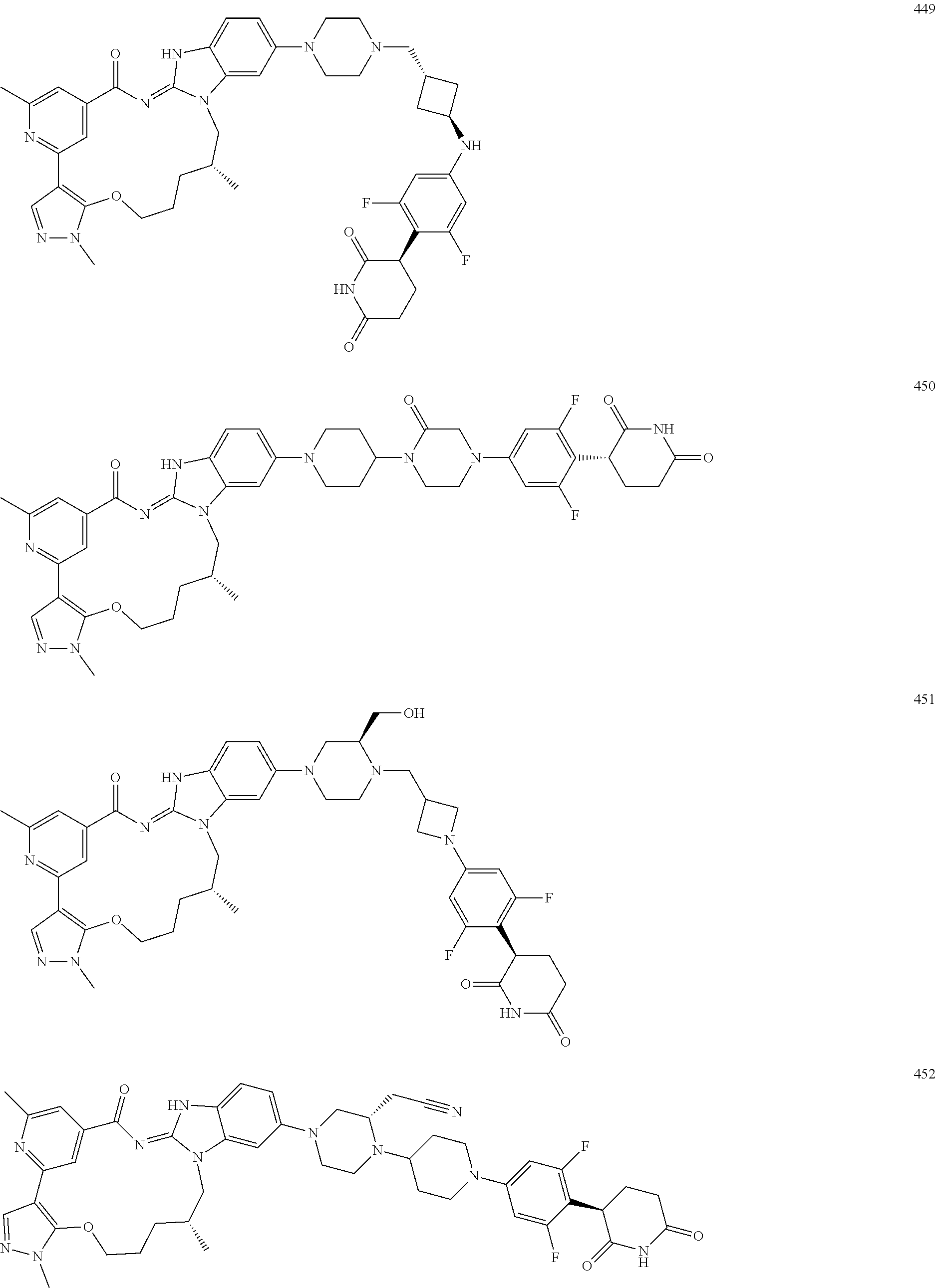

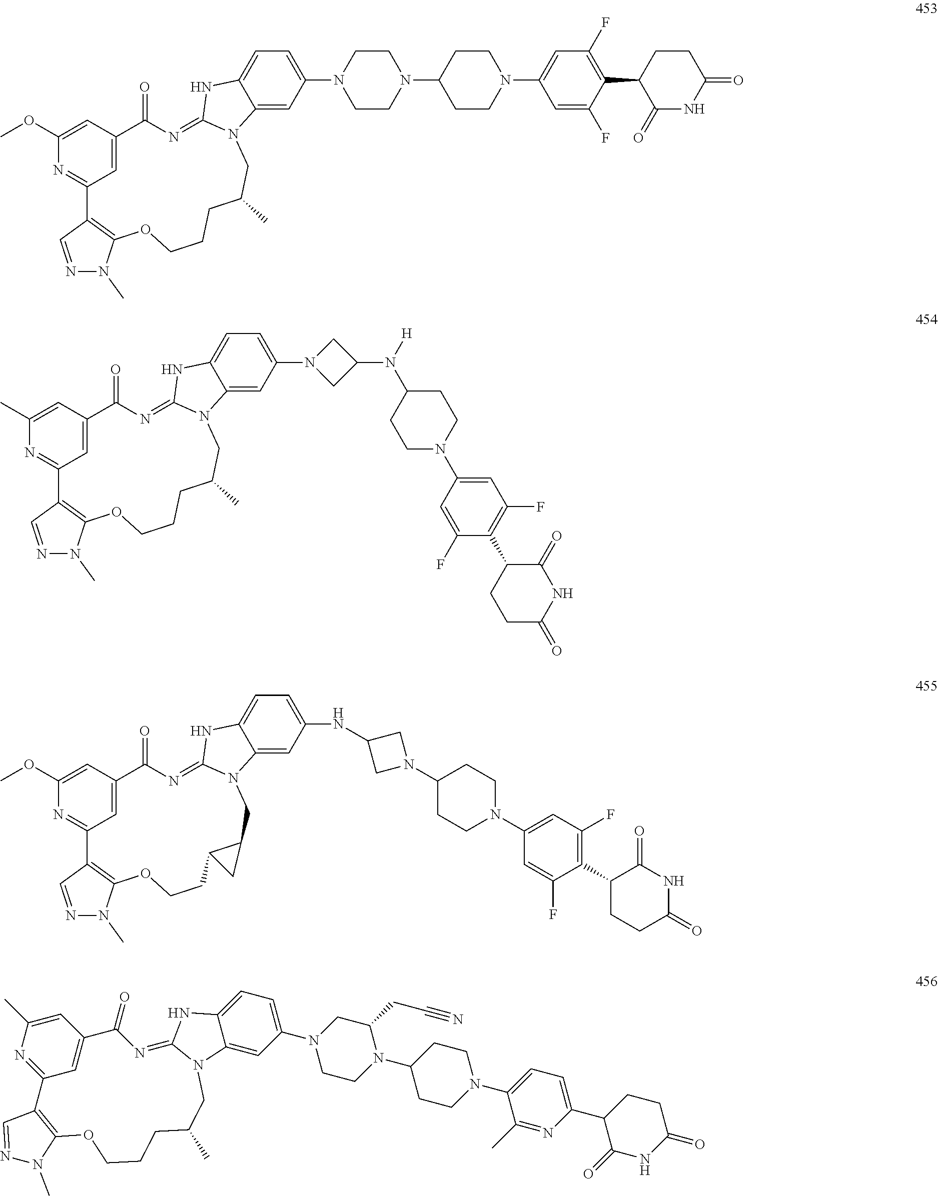 [SMILES: COc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CC(NC4CCN(c5cc(F)c([C@H]6CCC(=O)NC6=O)c(F)c5)CC4)C3)cc21 ; COc1cc2cc(n1)-c1cnn(C)c1OCC[C@H]1C[C@@H]1CN1/C(=N/C2=O)Nc2ccc(NC3CN(C4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5C)CC4)[C@@H](CC#N)C3)cc21]

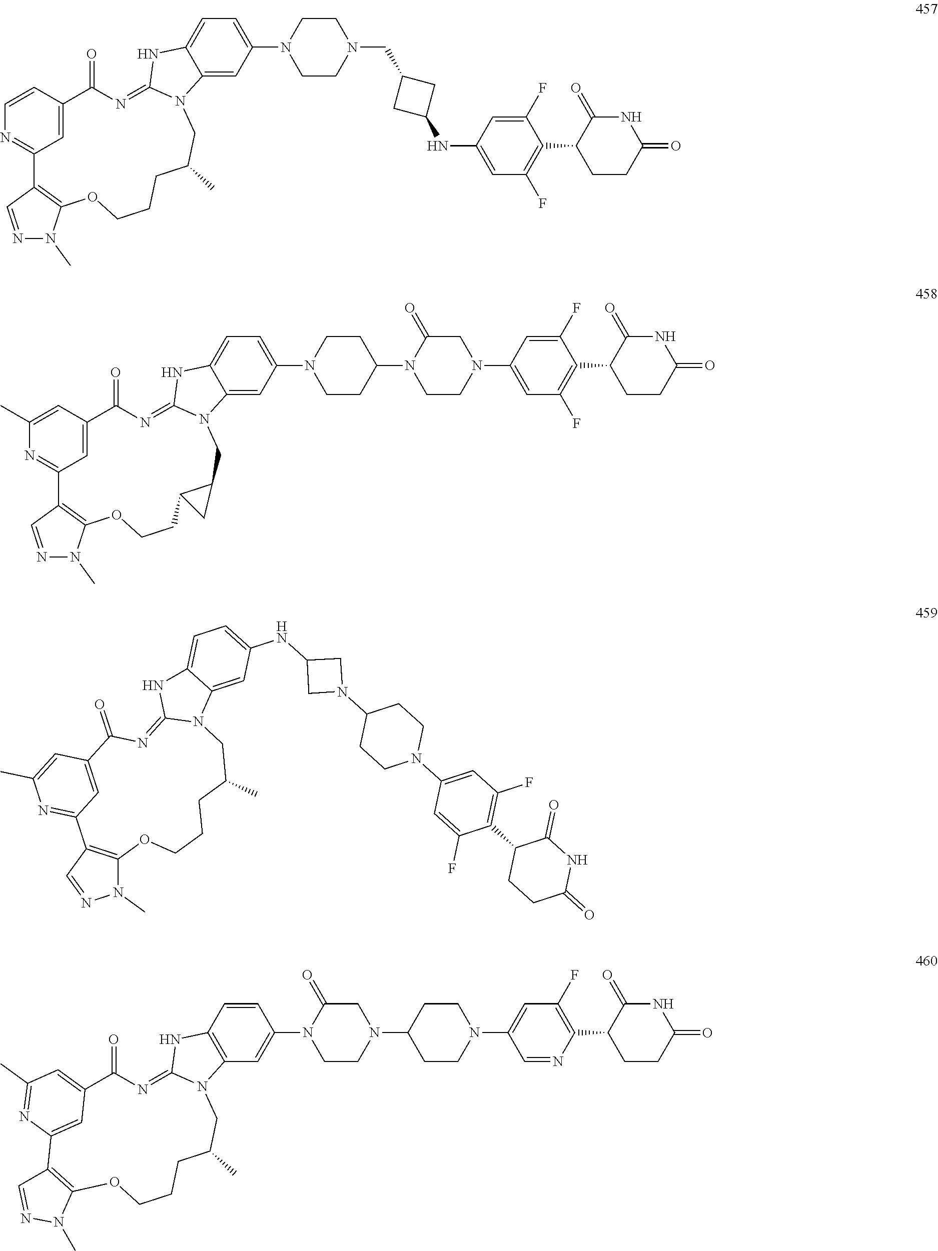 [SMILES: C[C@@H]1CCCOc2c(cnn2C)-c2cc(ccn2)C(=O)/N=C2\Nc3ccc(N4CCN(C[C@H]5C[C@H](Nc6cc(F)c([C@H]7CCC(=O)NC7=O)c(F)c6)C5)CC4)cc3N2C1 ; Cc1cc2cc(n1)-c1cnn(C)c1OCC[C@H]1C[C@@H]1CN1/C(=N/C2=O)Nc2ccc(N3CCC(N4CCN(c5cc(F)c([C@H]6CCC(=O)NC6=O)c(F)c5)CC4=O)CC3)cc21 ; Cc1cc2cc(n1)-c1cnn(C)c1OCCC[C@@H](C)CN1/C(=N/C2=O)Nc2ccc(NC3CN(C4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(N3CCN(C4CCN(c5cnc([C@H]6CCC(=O)NC6=O)c(F)c5)CC4)CC3=O)cc21]

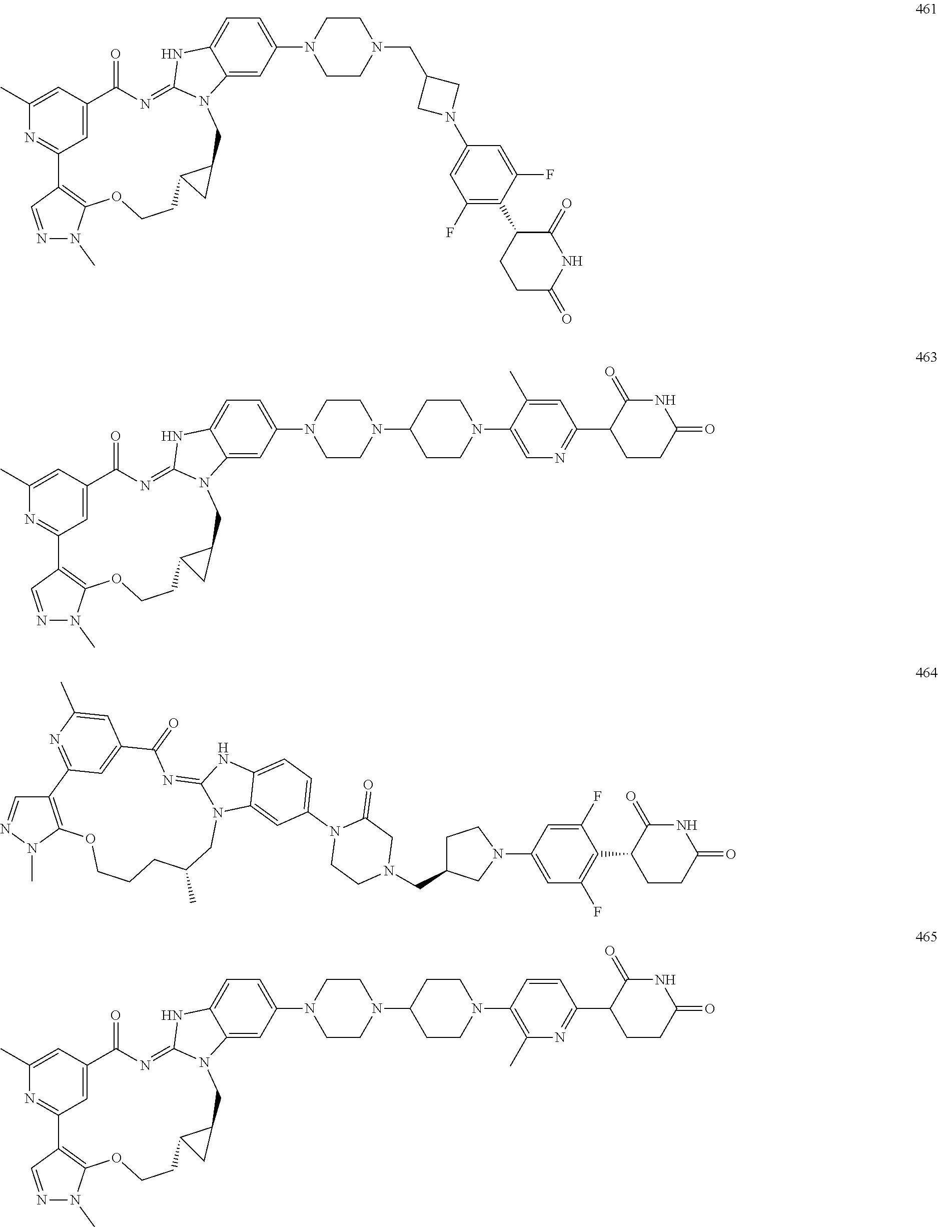

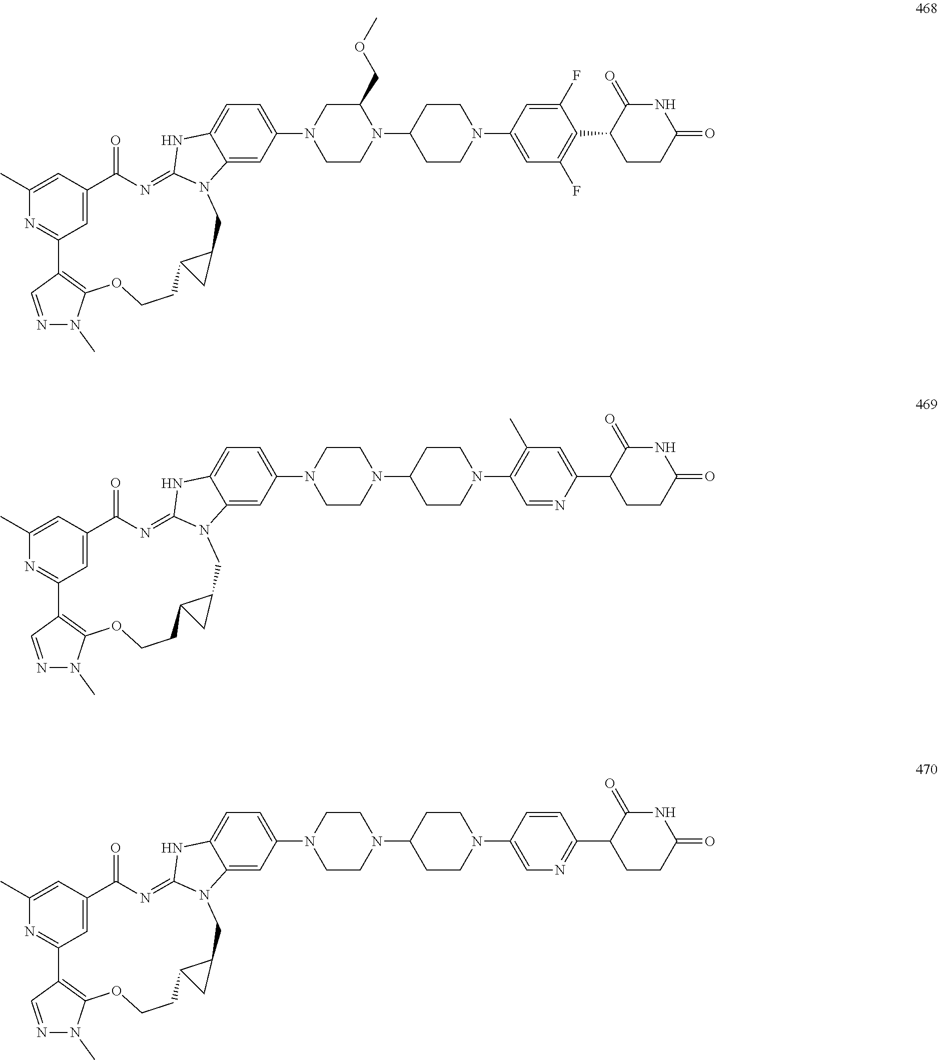 [SMILES: COC[C@@H]1CN(c2ccc3c(c2)N2C[C@H]4C[C@@H]4CCOc4c(cnn4C)-c4cc(cc(C)n4)C(=O)/N=C/2N3)CCN1C1CCN(c2cc(F)c([C@H]3CCC(=O)NC3=O)c(F)c2)CC1 ; Cc1cc2cc(n1)-c1cnn(C)c1OCC[C@@H]1C[C@H]1CN1/C(=N/C2=O)Nc2ccc(N3CCN(C4CCN(c5cnc(C6CCC(=O)NC6=O)cc5C)CC4)CC3)cc21 ; Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5)CC4)CC3)cc21]

Aspect 53. A pharmaceutical composition comprising a compound of any one of Aspects 1-52 or a pharmaceutically acceptable salt, stereoisomer, tautomer or prodrug thereof, together with a pharmaceutically acceptable excipient.

Aspect 54. A method of treating a disease that can be affected by EGFR modulation, comprises administrating a subject in need thereof an effective amount of a compound of any one of Aspects 1-52 or a pharmaceutically acceptable salt, stereoisomer, tautomer or prodrug thereof.

Aspect 55. The method of Aspect 52, wherein the disease is selected from cancer, preferred pancreatic cancer, breast cancer, glioblastoma multiforme, head and neck cancer, or non-small cell lung cancer.

Aspect 56. Use of a compound of any one of Aspects 1-52 or a pharmaceutically acceptable salt, stereoisomer, tautomer or prodrug thereof in the preparation of a medicament for treating a disease that can be affected by EGFR modulation.

Aspect 57. The use of Aspect 56, wherein the disease is cancer, preferred pancreatic cancer, breast cancer, glioblastoma multiforme, head and neck cancer, or non-small cell lung cancer.

We surprisingly found that the compounds with linker like

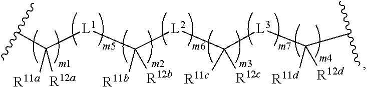

moiety between EGFR binder moieties and E3 ligase Ligand moieties have better activity than other compounds. Hereby the better activity means the compounds have better Degradation activity, further means the compounds could degrade the target protein (hereby means the EGFR protein) with high efficiency. The DC₅₀ is lower than 20 nm, preferably lower than 10 nm, more preferably lower than 5 nm, 3 nm, 2 nm or 1 nm. The Dmax is higher than 60%, preferably higher than 70% or 80%. Preferably m1, m2, m3 and m4 are each independently 0, 1 or 2 and provided that m1+m2+m3+m4≤4, preferably m1+m2+m3+m4=1, 2 or 3, more preferably m1+m2+m3+m4=1 or 2.

In some particular embodiments, the linker is

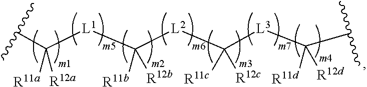

L¹ is independently selected from —O—, —NR^{a}—,

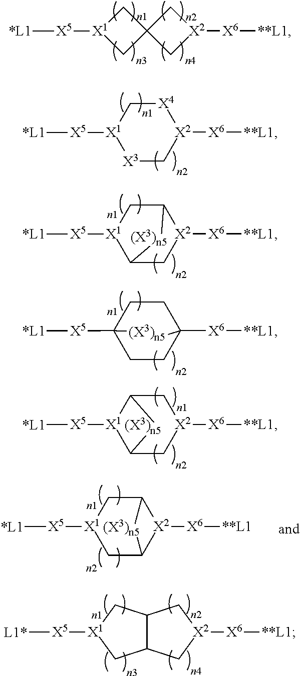

wherein each of said

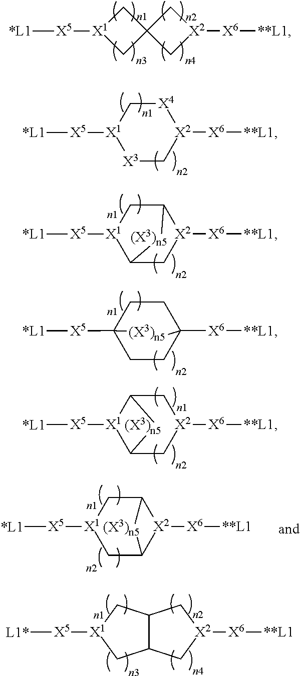

is optionally substituted with at least one R^{L1c};
wherein *^{L1} refers to the position attached to the

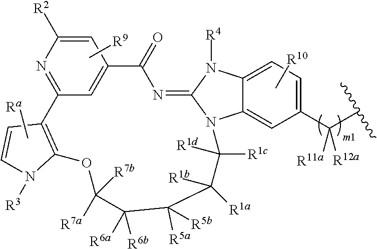

moiety, and **^{L1} refers to the position attached to the

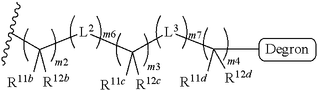

moiety;
L² is independently selected from —O—, —NR^{a}—,

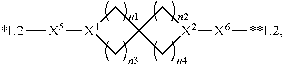

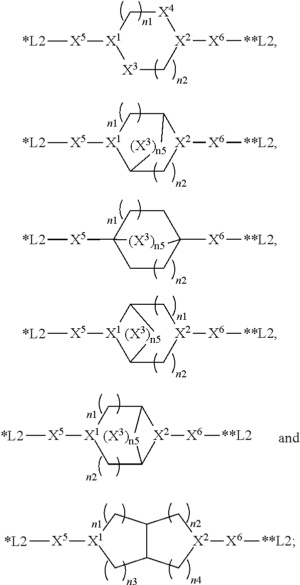

wherein each of said

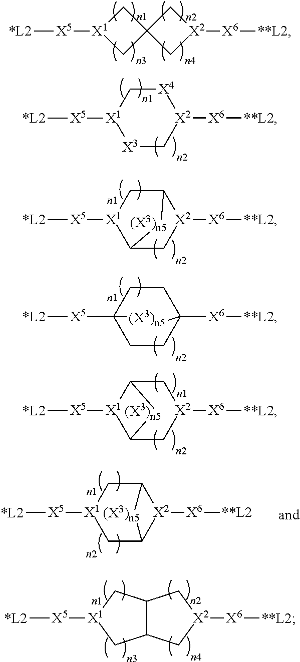

is optionally substituted with at least one R^{L2c};
wherein *^{L2} refers to the position attached to the

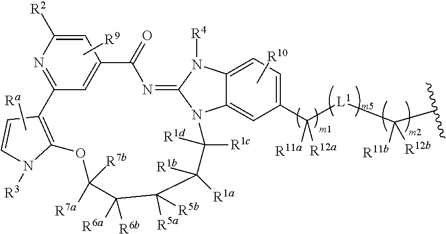

moiety, and
*^{L2} refers to the position attached to the

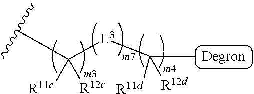

moiety;
L³ is independently selected from —O—, —NR^{a}—,

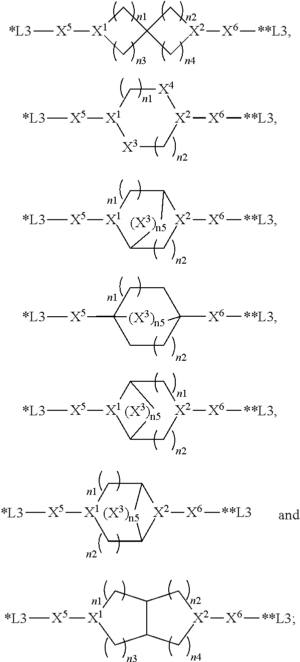

wherein each of said

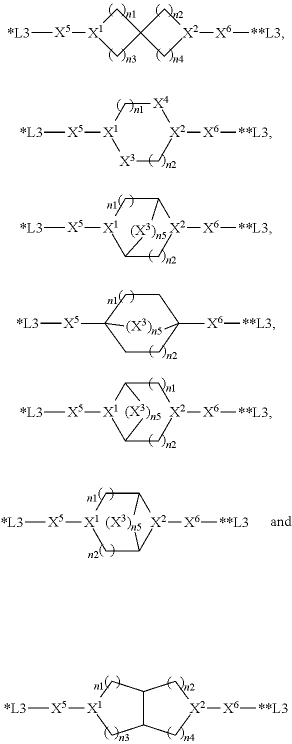

is optionally substituted with at least one R^{L3c};
R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, L⁴, Z¹, Z², Z³, X⁷, X⁸, m1, m2, m3, m4, m5, m6, m7 and n6 are defined as the previous aspects.

In some particular embodiments, for any one of L¹, L² or L¹, when X¹ is N, X⁵ is single bond, absence, —C(O)—; and/or when X² is N, X⁶ is single bond, absence, —C(O)—;
when L¹ is

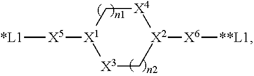

when X¹ is N, X⁵ is single bond, absence, —C(O)— and/or X³ is —CR^{a7}R^{b7}; when X² is N, X⁶ is single bond, absence; —C(O)—, and/or X⁴ is —CR^{a7}R^{b7};
when L² is

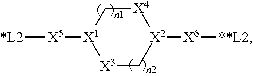

when X¹ is N, X⁵ is single bond, absence, —C(O)—; and/or X³ is —CR^{a7}R^{b7}; when X² is N, X⁶ is single bond, absence: —C(O)—, and/or X⁴ is —CR^{a7}R^{b7};
when L³ is

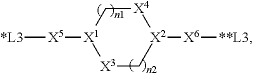

when X¹ is N, X⁵ is single bond, absence, —C(O)—, and/or X³ is —CR^{a7}R^{b7}; when X² is N, X¹ is single bond, absence: —C(O)—, and/or X⁴ is —CR^{a7}R^{b7};
when L² is

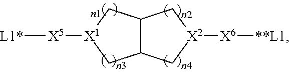

n1, n2, n3, and n4 are each independently 1, 2 or 3;
when L¹ is

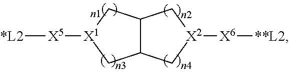

n1, n2, n3, and n4 are each independently 1, 2 or 3;
when L¹ is

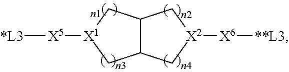

n1, n2, n3, and n4 are each independently 1, 2 or 3.

In some particular embodiments, L¹ is selected from —O—, —N(R^{a})—,

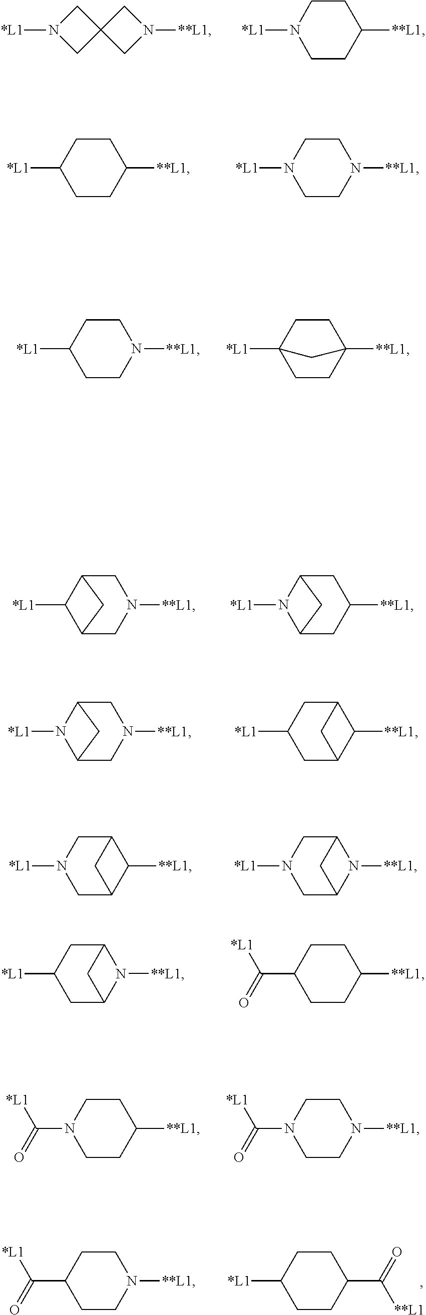

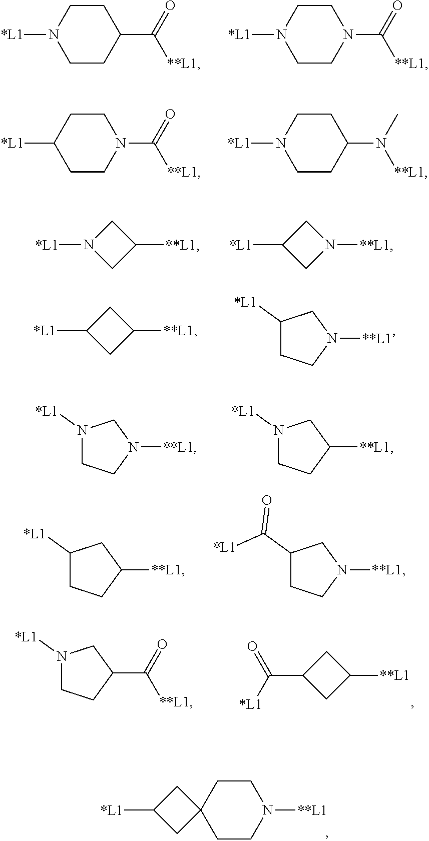

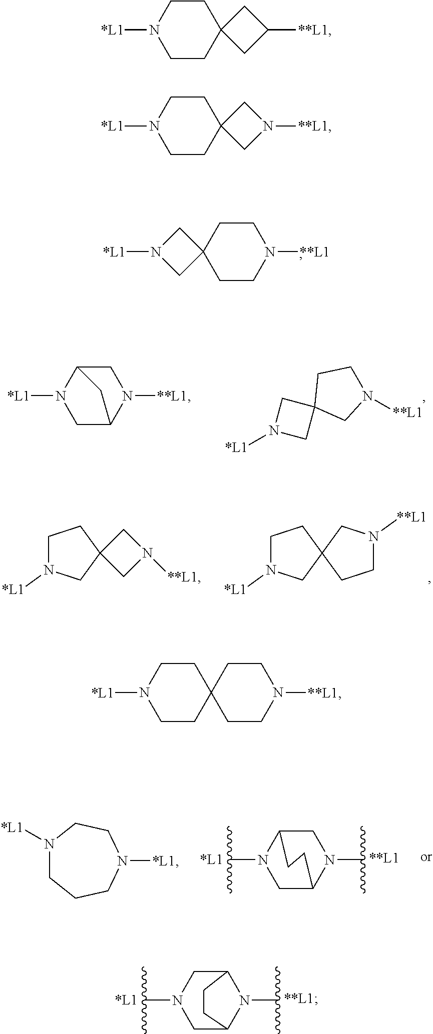

wherein each of said

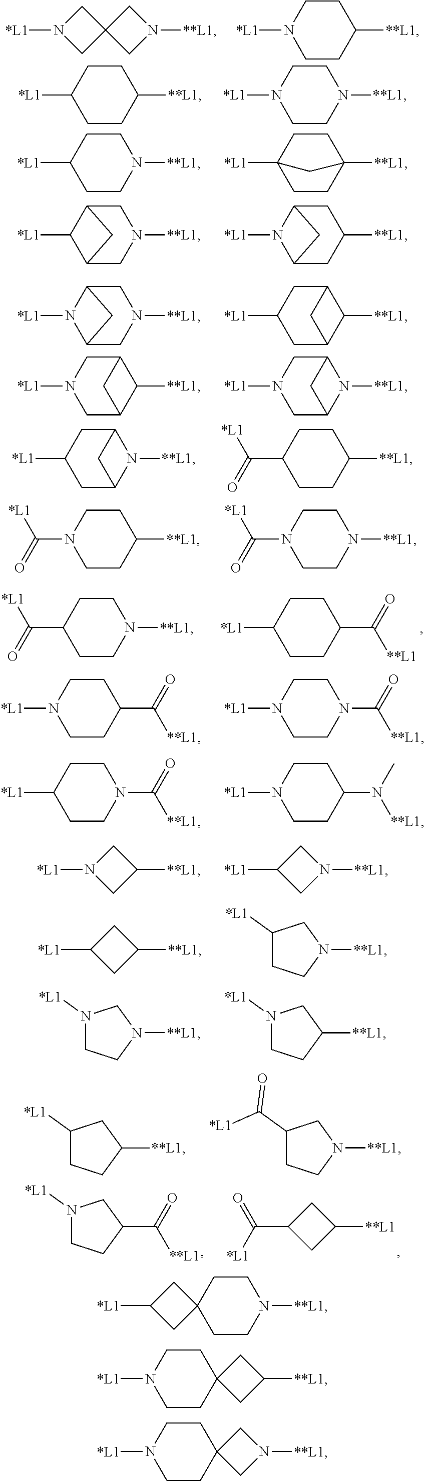

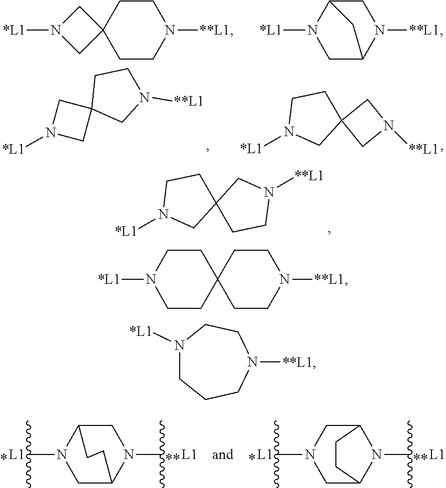

is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl:
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

In some particular embodiments, L¹ is selected from —O—, —N(CH₃)—,

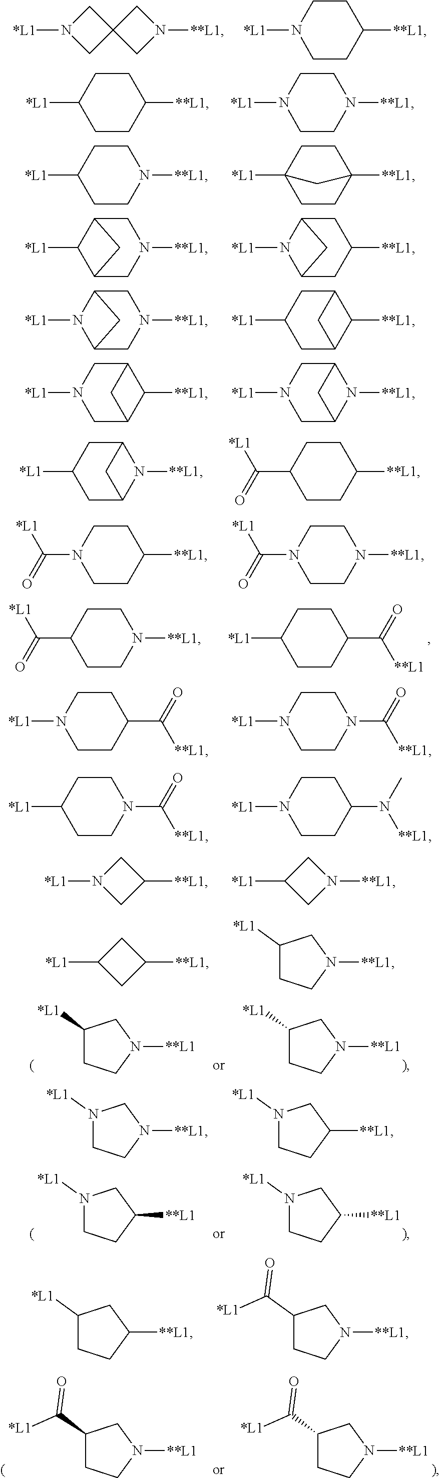

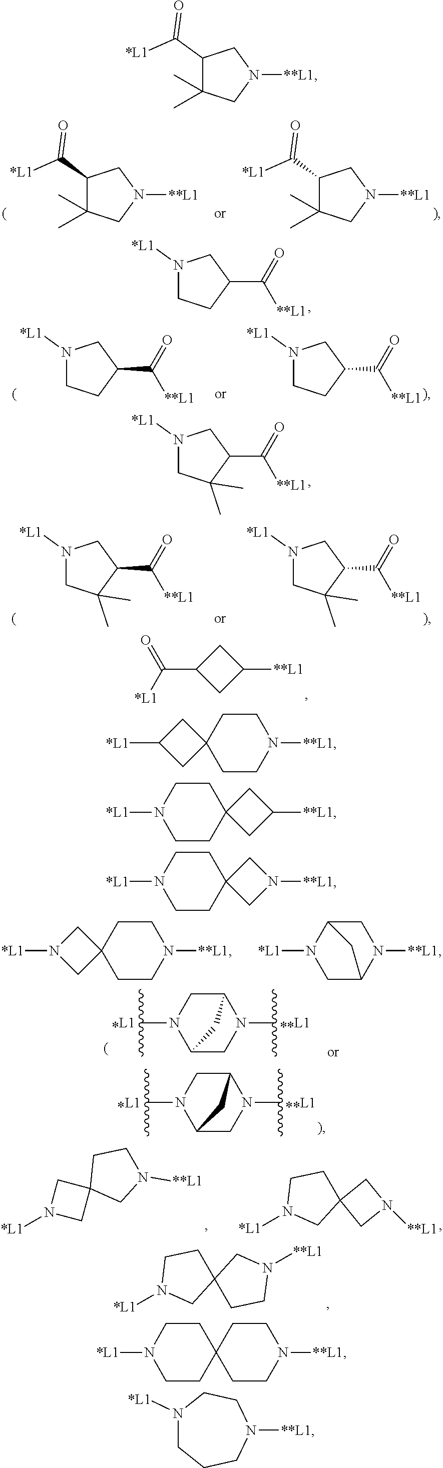

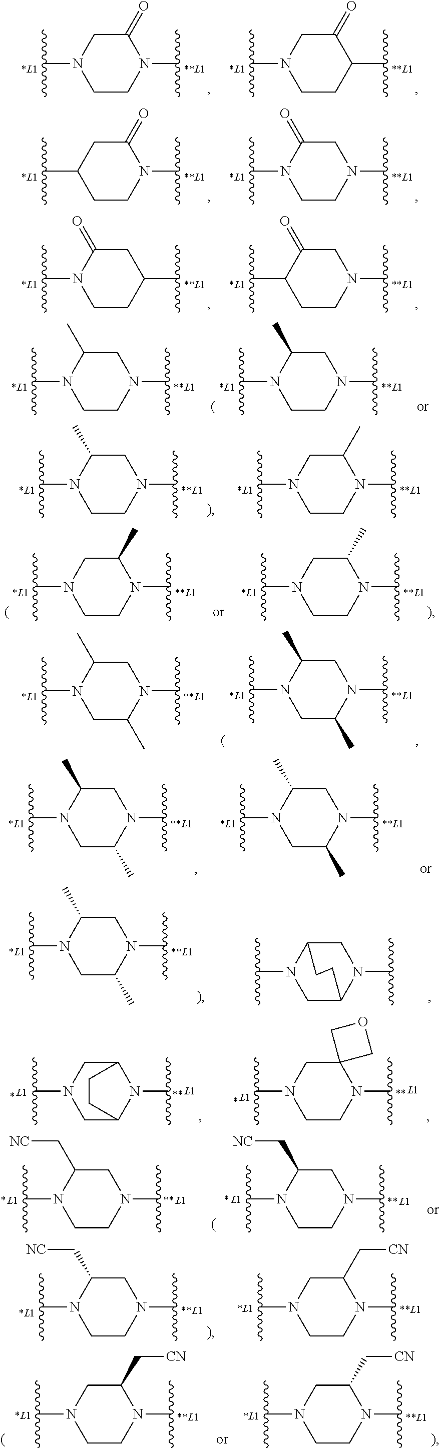

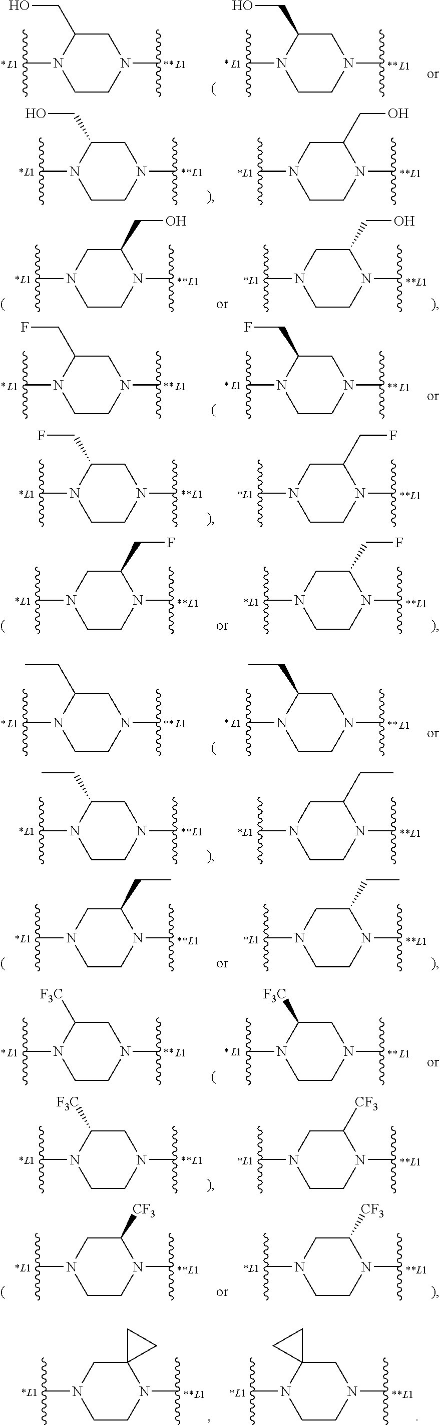

In some particular embodiments, L² is selected from —O—, —N(R^{a})—,

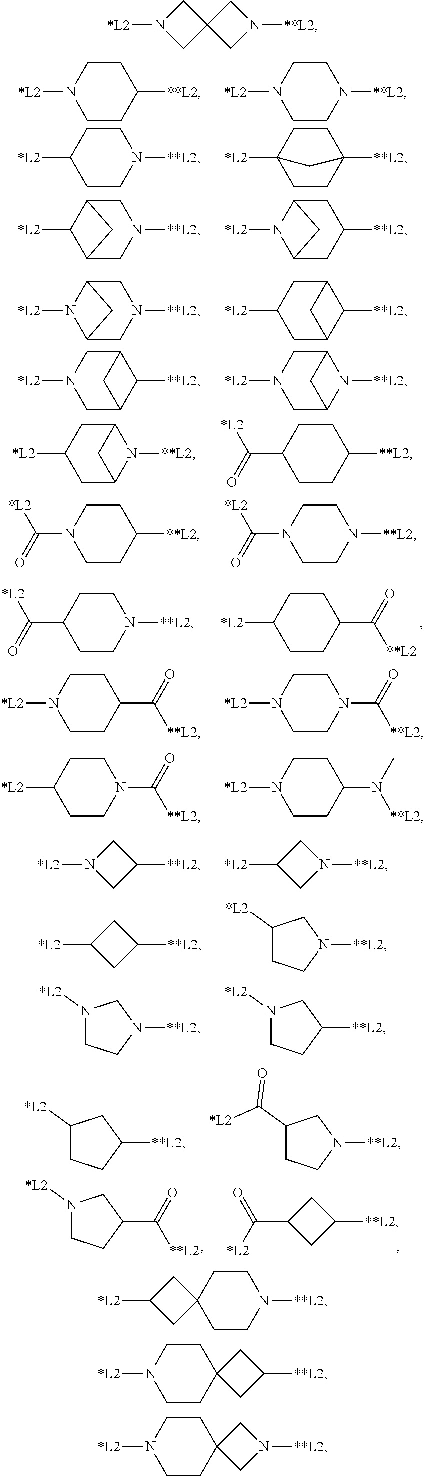

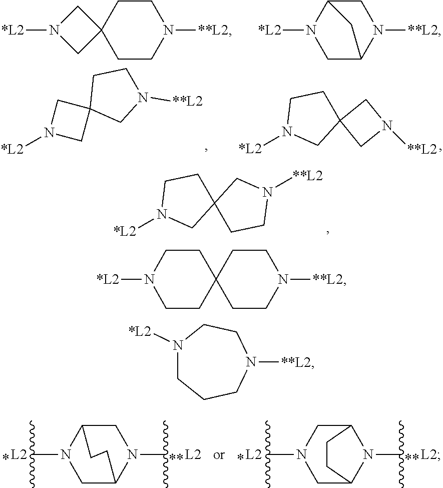

wherein each of said

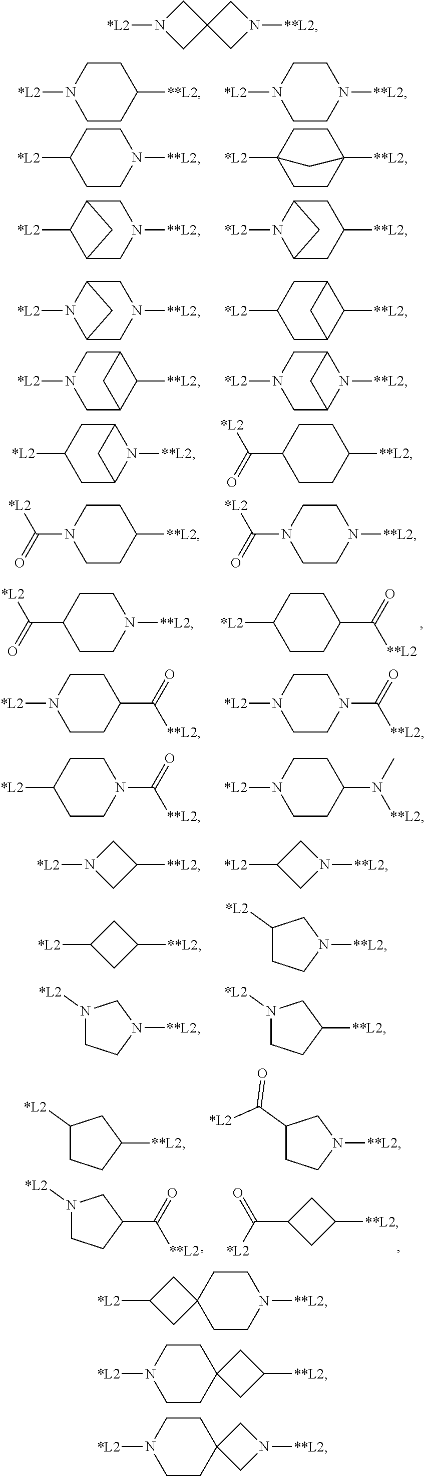

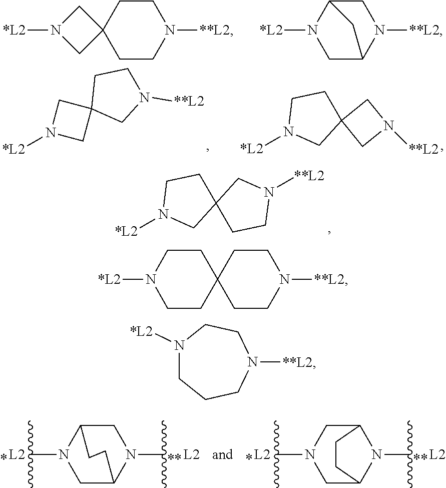

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

In some particular embodiments, L² is selected from —O—, —N(CH₃)—,

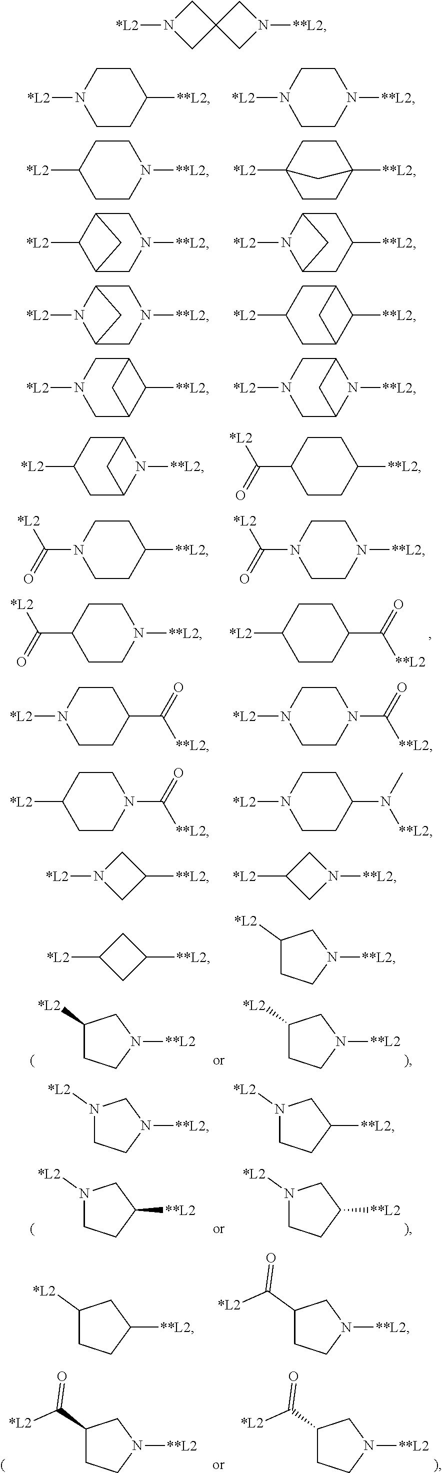

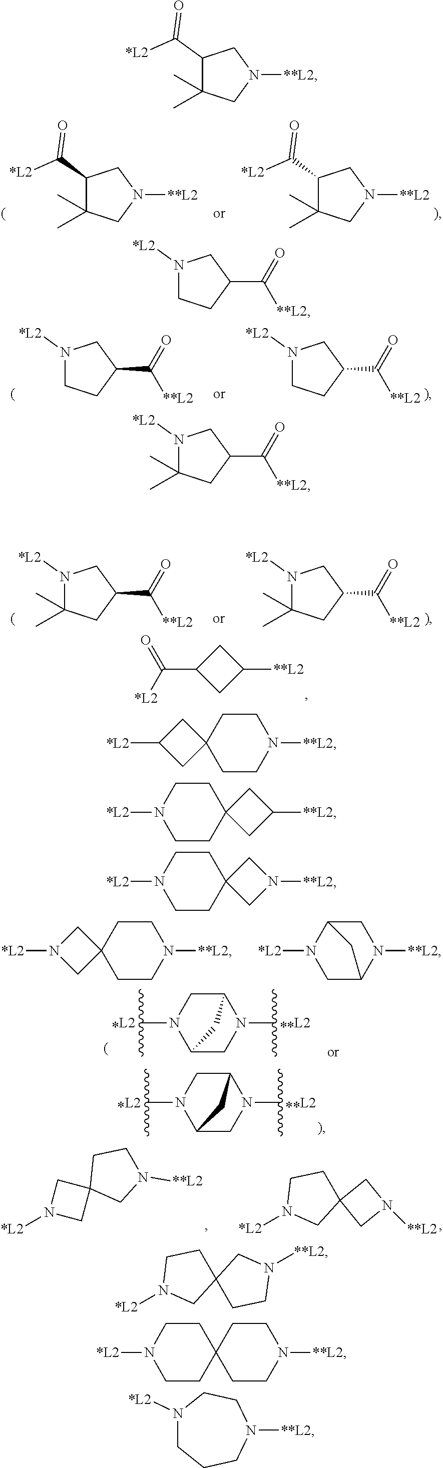

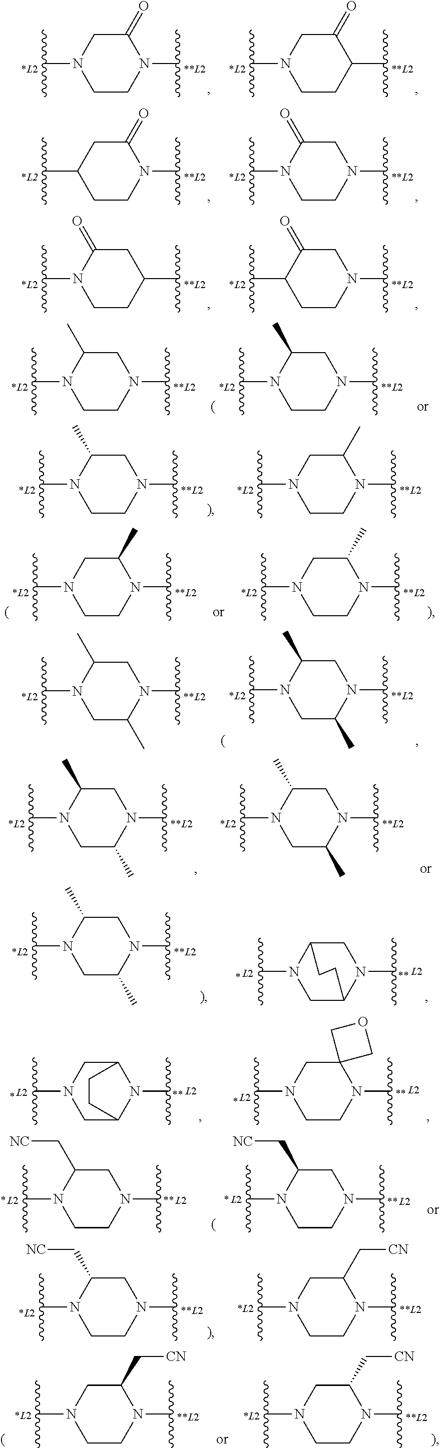

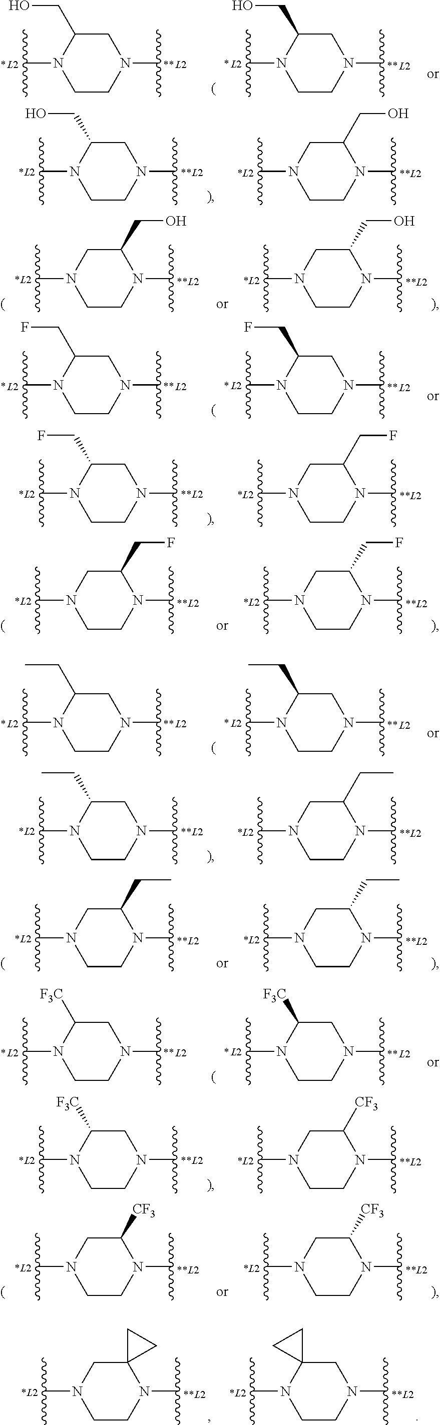

In some particular embodiments, L³ is selected from —O—, —N(R^{a})—,

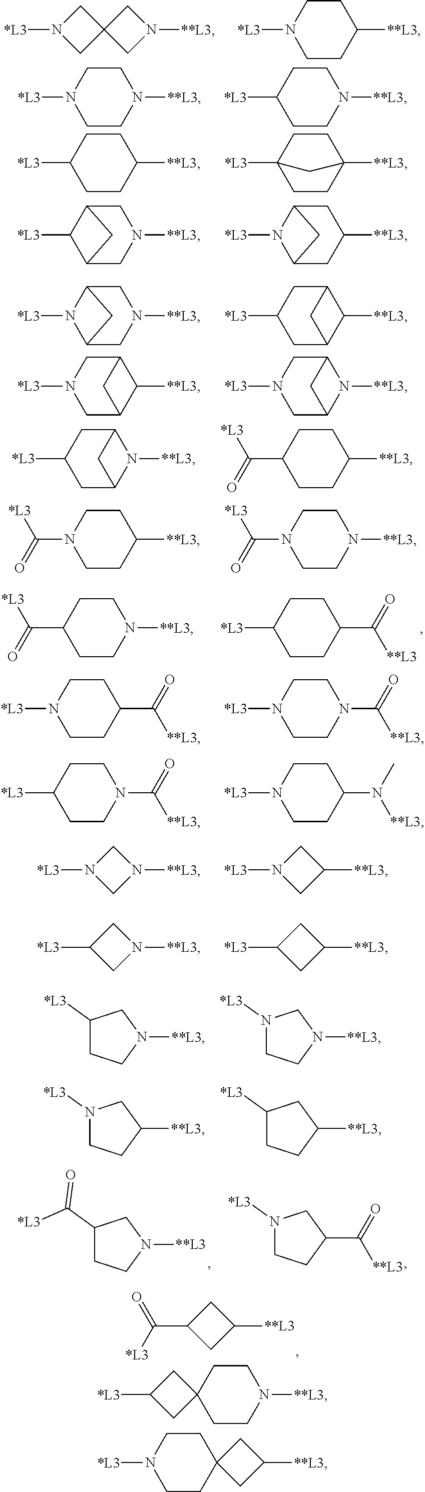

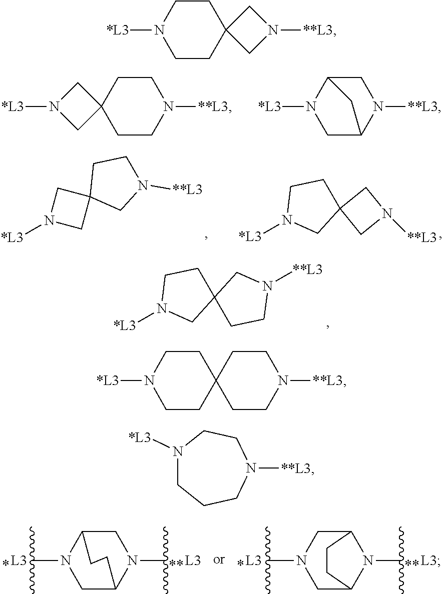

wherein each of said

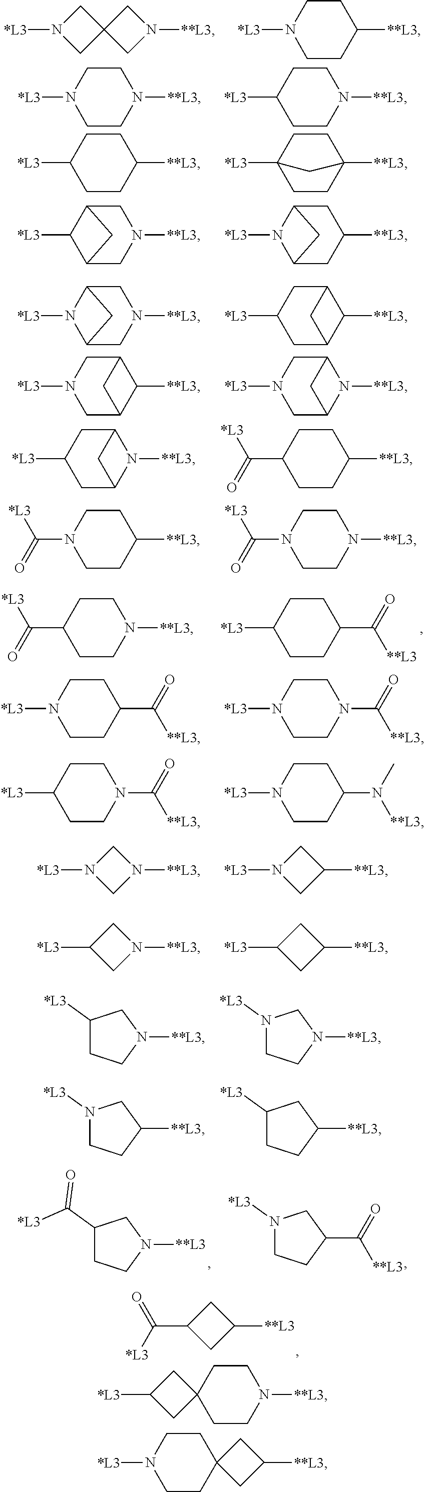

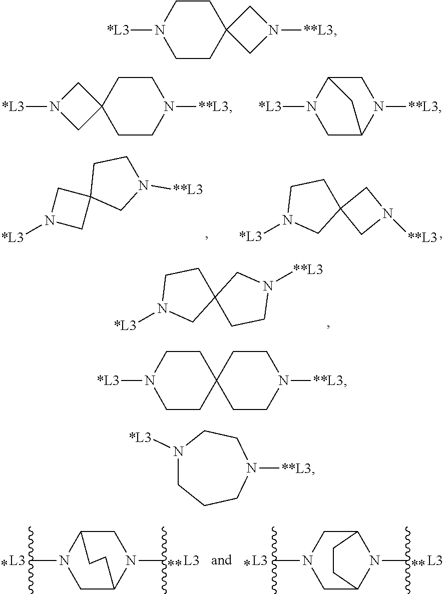

is optionally substituted with at least one R^{L3c}; each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

In some particular embodiments, L¹ is selected from —O—, —N(CH₃)—,

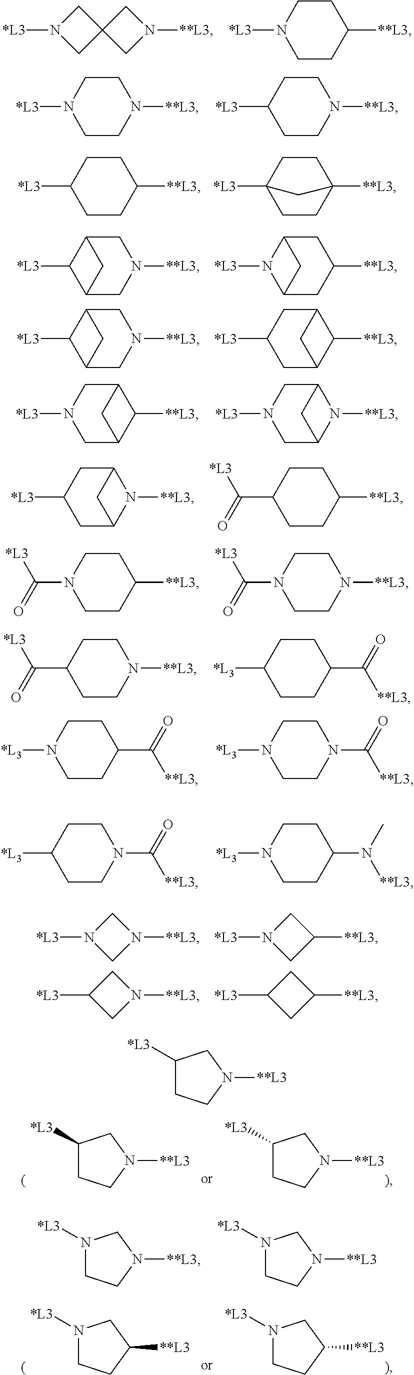

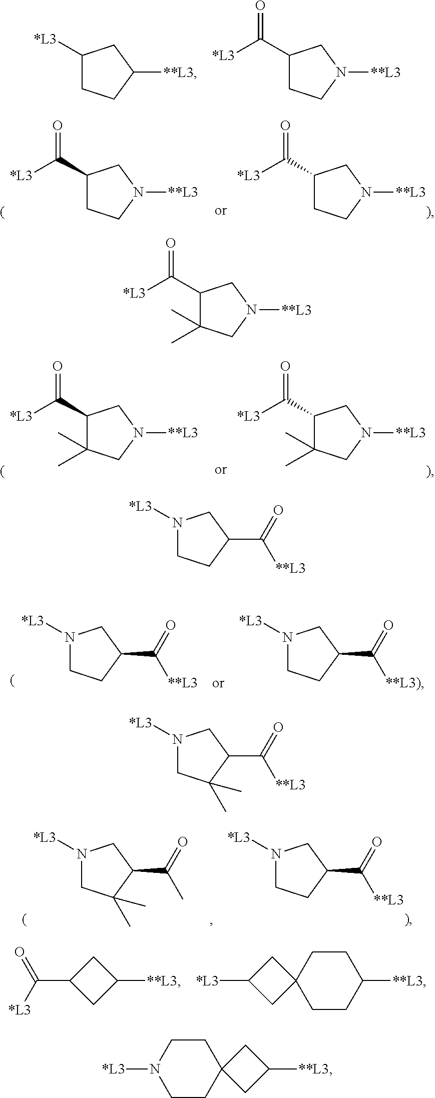

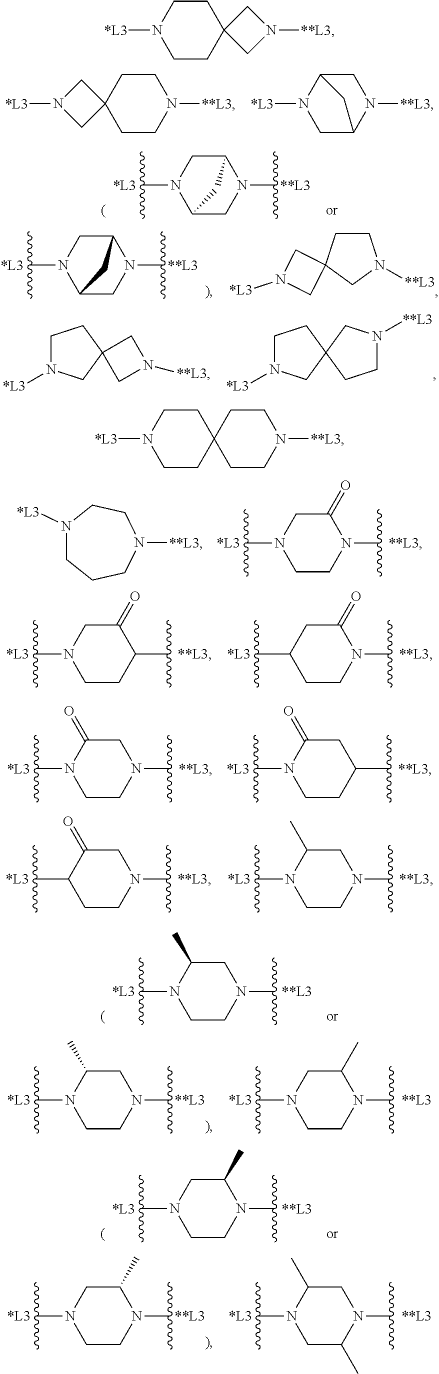

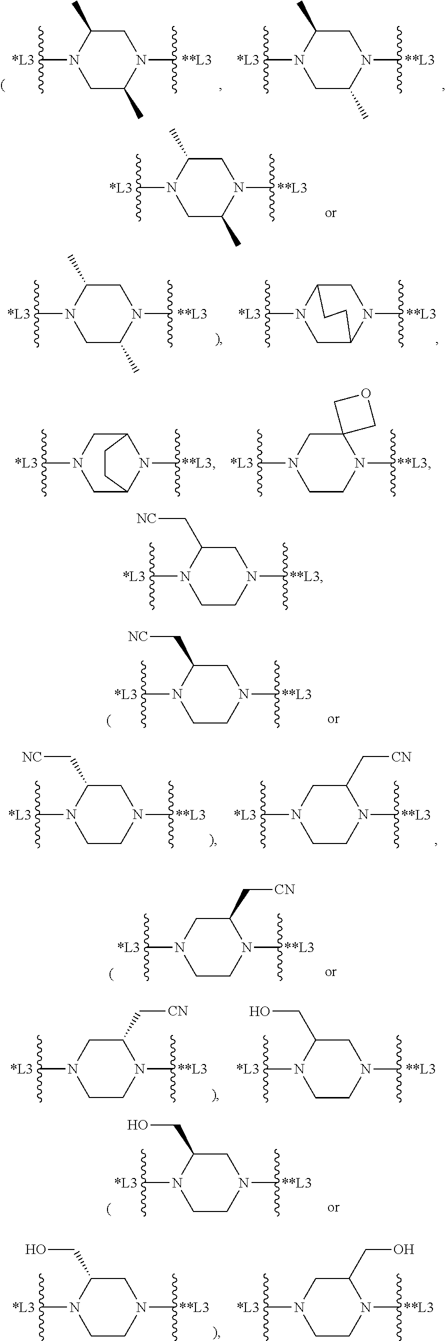

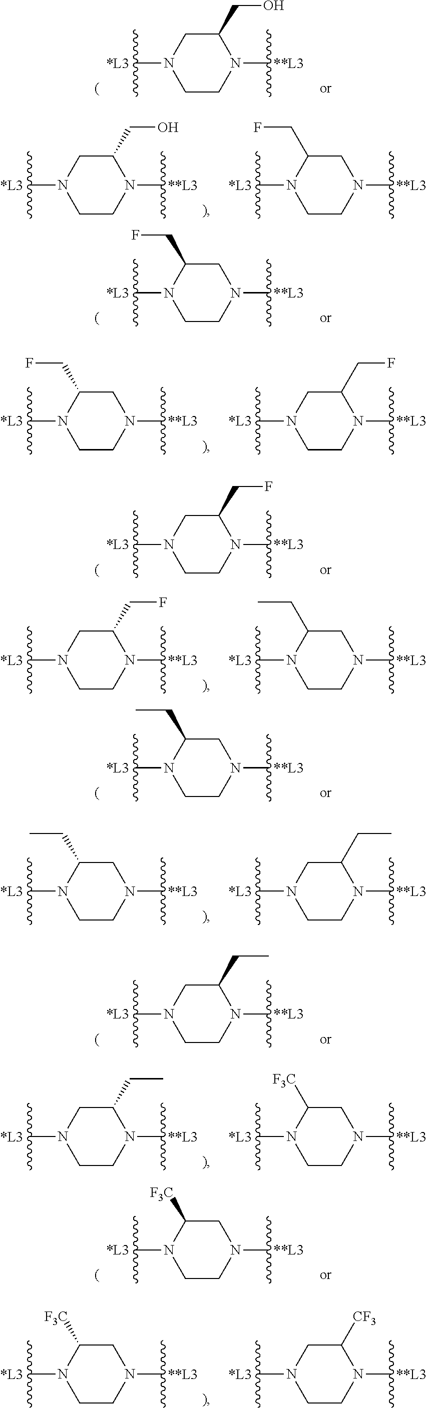

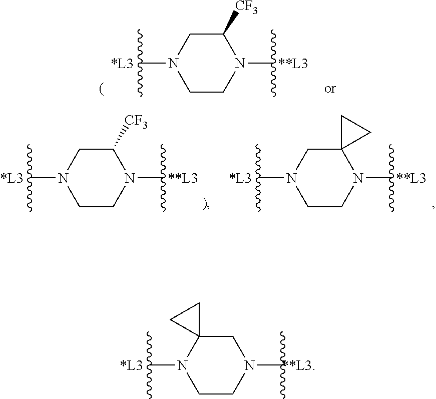

In some particular embodiments,

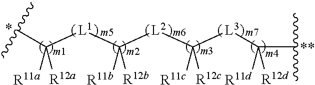

moiety is selected from

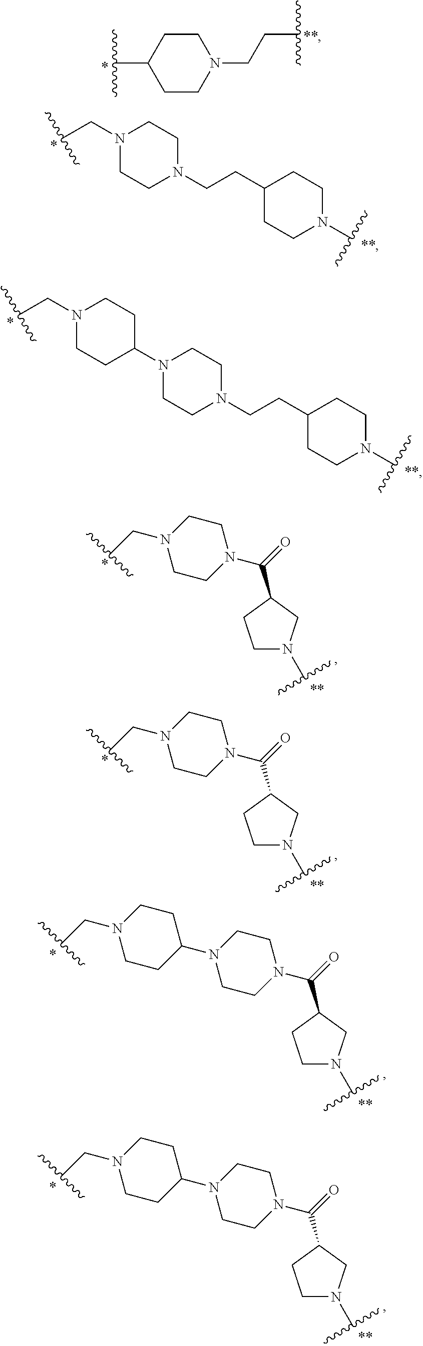

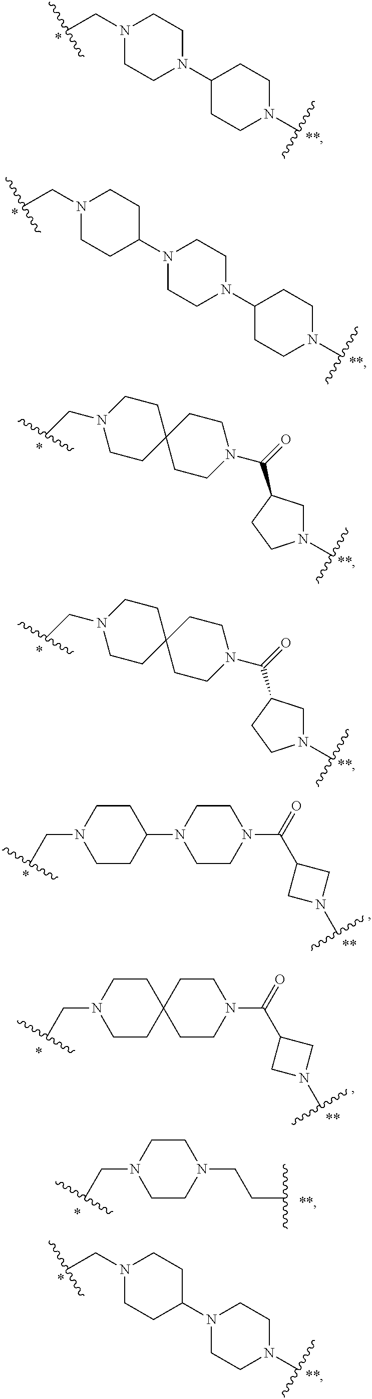

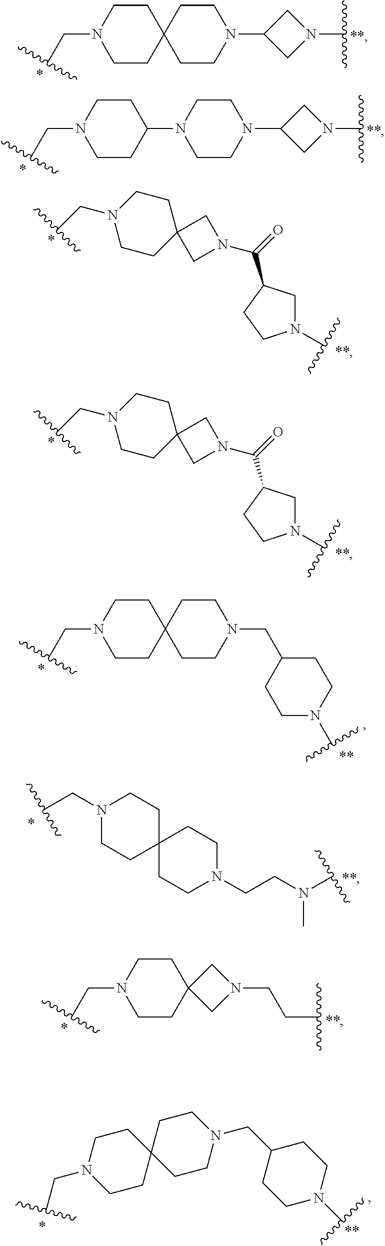

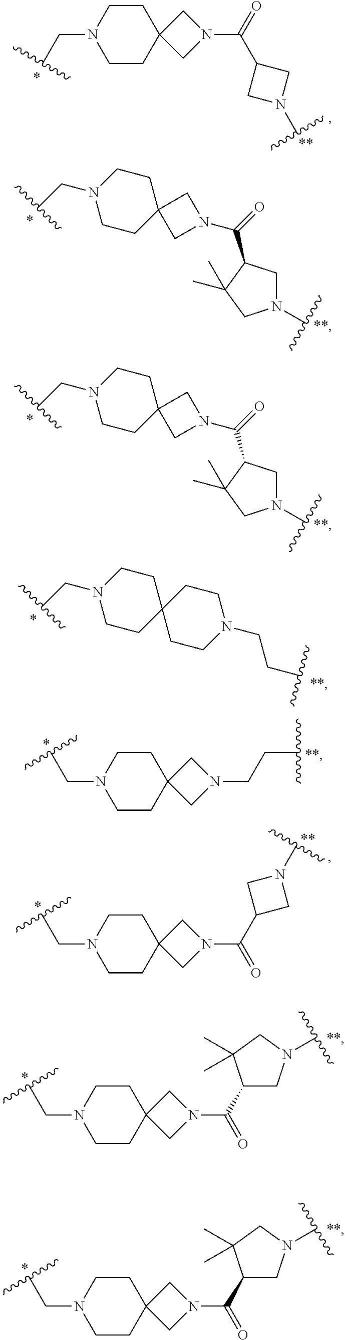

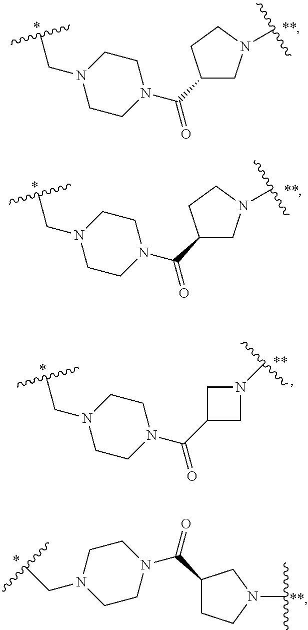 [SMILES: *CN1CCN(C(=O)[C@@H]2CCN(*)C2)CC1 ; *CN1CCN(C(=O)[C@H]2CCN(*)C2)CC1 ; *CN1CCN(C(=O)C2CN(*)C2)CC1 ; *CN1CCN(C(=O)[C@@H]2CCN(*)C2)CC1]

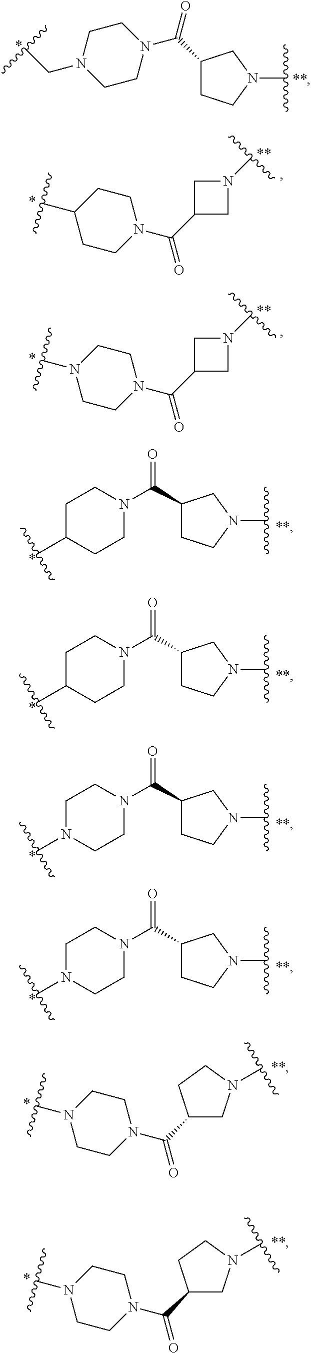

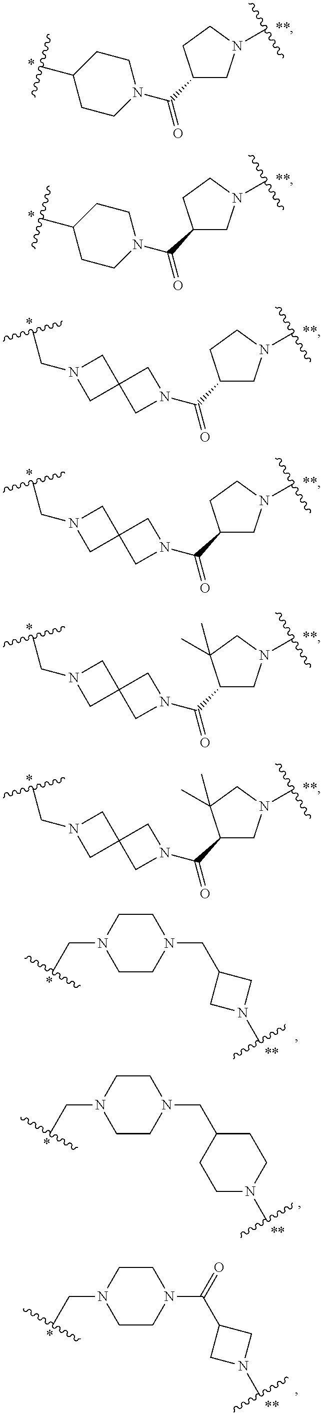

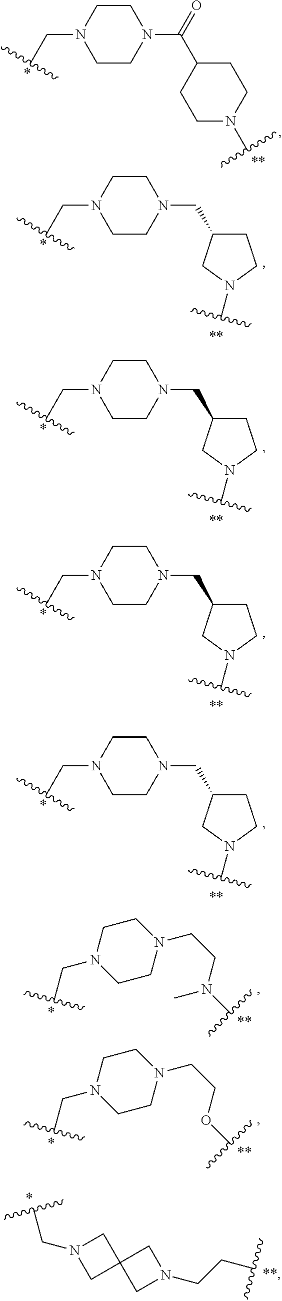

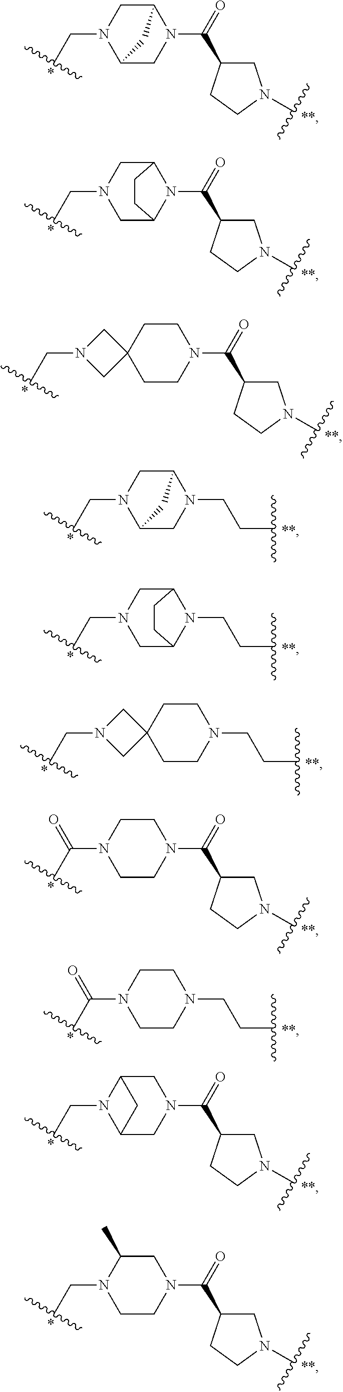

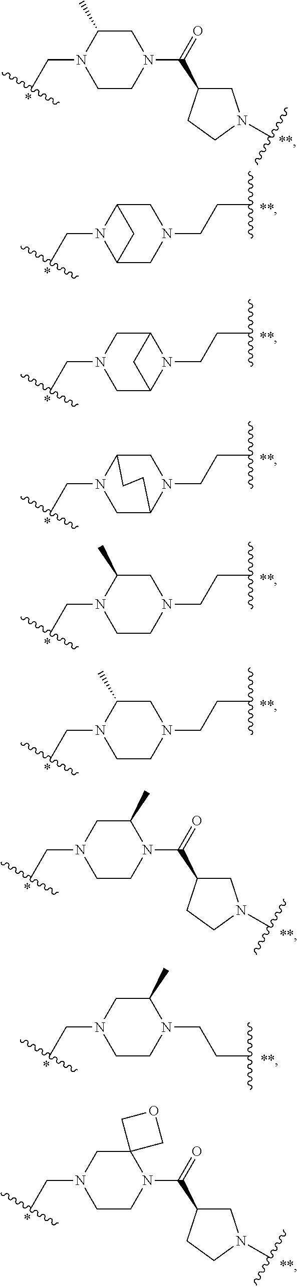

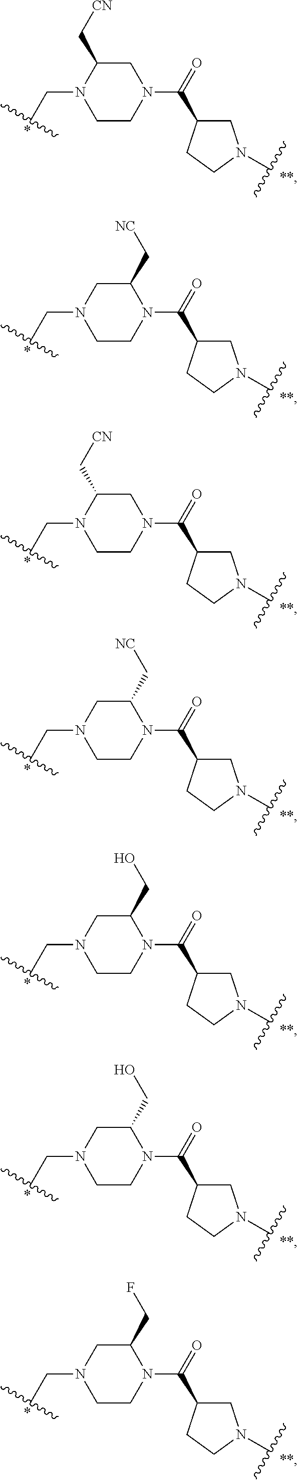

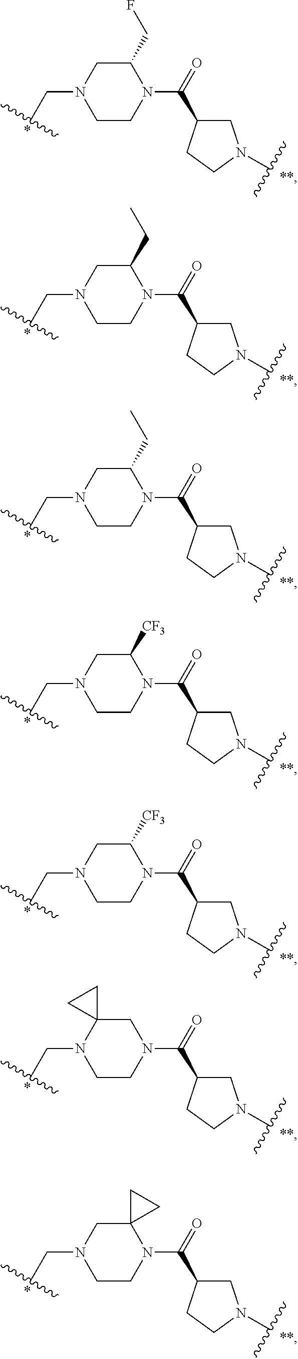

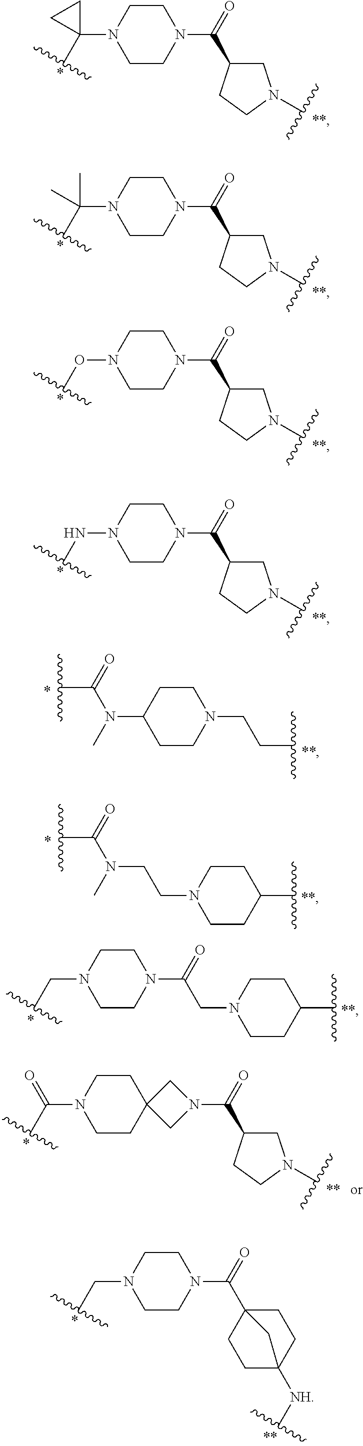

We also surprisingly found that the compounds with

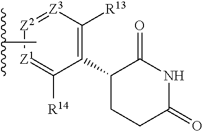

E3 ligase Ligand moieties have excellent binding activity to the E3 ligase; further the compounds with

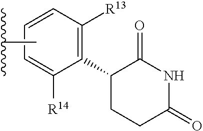

E3 ligase Ligand moieties have excellent binding activity to the E3 ligase.

For anyone of the parameters, including but no limiting to m1, m2, m3, m4, m5, m6, m7, n1, n2, n3, n4, n5, n6, n7 and n8, when anyone of the parameters is 0, it means the group that parameter restricts is absence, hydrogen or a single bond.

In some particular embodiments, for any one of L¹, L² or L, when X¹ is N, X⁵ is single bond, absence, —C(O)—; and/or when X² is N, X⁶ is single bond, absence, —C(O)—;
when L¹ is

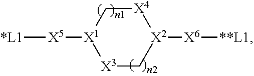

when X¹ is N, X⁵ is single bond, absence, —C(O)—; and/or X³ is —CR^{a7}R^{b7}; when X² is N, X⁶ is single bond, absence; —C(O)—, and/or X⁴ is —CR^{a7}R^{b7};
when L² is

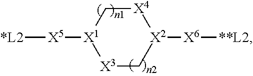

when X¹ is N, X¹ is single bond, absence, —C(O)—; and/or X³ is —CR^{a7}R^{b7}; when X² is N, X⁶ is single bond, absence; —C(O)—, and/or X⁴ is —CR^{a7}R^{b7};
when L¹ is

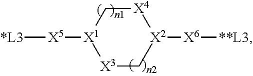

when X¹ is N, X⁵ is single bond, absence, —C(O)—; and/or X³ is —CR^{a7}R^{b7}; when X² is N, X⁶ is single bond, absence: —C(O)—, and/or X⁴ is —CR^{a7}R^{b7};
when L¹ is

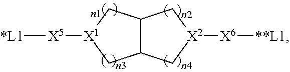

n1, n2, n3, and n4 are each independently 1, 2 or 3;
when L² is

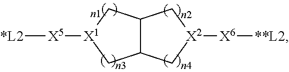

n1, n2, n3, and n4 are each independently 1, 2 or 3;
when L¹ is

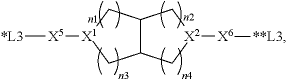

n1, n2, n3, and n4 are each independently 1, 2 or 3.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C═C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl:
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from.
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N. O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group, the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase: size exclusion; ion exchange; high, medium and low pressure liquid chromatography methods and apparatus: small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel. E and Wilen. S. Stereochemistry of Organic Compounds. New York: John Wiley & Sons. Inc., 1994; Lochmuller. C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W. Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “treated”, “treating” or “treatment” as used herein also generally refers to the acquisition of the desired pharmacological and/or physiological effect. The effect may be prophylactic according to the prevention of the disease or its symptoms in whole or in part; and/or may be therapeutic according to the partial or complete stabilization or cure of the disease and/or the side effect due to the disease. As used herein, “treated”, “treating” or “treatment” encompasses any treatment for the disease of a patient, including: (a) prevention of the disease or condition in the patient that may be predisposed to the disease or condition but has not yet been diagnosed; (b) inhibition of the symptoms of the disease, i.e., preventing its development: or (c) remission of the symptoms of the disease, i.e., causing regression of the disease or symptoms in whole or in part.

The term “effective amount” or “therapeutically effective amount” refers to an amount of the active ingredient, such as compound that, w % ben administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless otherwise specified, the percentages, proportions, ratios or parts used in the present application are by weight or volume. The amount used in the present application is a weight or volume amount. It can be determined easily by those skilled in the art.

Hereinafter, the present application will demonstrate the beneficial effects of the present application by way of examples. Those skilled in the art will recognize that these examples are illustrative and not restrictive. These examples will do not limit the scope of the present application in any way. The experimental methods described in the following examples, unless otherwise specified, are conventional methods; the reagents and materials, unless otherwise specified, are commercially available.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column; Poroshell 120 EC-C18, 4.6×50 mm, 2.7 pm Gradient method; Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2.2′-Bis(diphenylphosphino)-1,1′-BINAPhthyl |
| Bn | benzyl |
| BnBr | Benzyl Bromide |
| Boc | t-Butyloxy carbonyl |
| BTEAC | Benzyltriethylammonium chloride |
| C:40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| TCFH | N,N,N′,N′-Tetramethylchloroformamidinium hexafluorophosphate |
| CDI | 1,1′-Carbonyldiimidazole |

### Example 5

### (R)-3-(2,6-difluoro-4-((R)-3-(4-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)piperazine-1-carbonyl)pyrrolidin-1-yl)phenyl)piperidine-2,6-dione

### Step 1: methyl (R)-1-(4-bromo-3,5-difluorophenyl)pyrrolidine-3-carboxylate

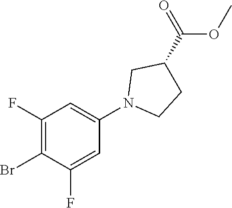 [SMILES: COC(=O)[C@@H]1CCN(c2cc(F)c(Br)c(F)c2)C1]

To the solution of 2-bromo-1,3-difluoro-5-iodobenzene (15 g, 47 mmol), methyl (R)-pyrrolidine-3-carboxylate hydrochloride (8.56 g, 51.7 mmol) and K₃PO₄ (20 g, 94 mmol) in 250 mL DMSO, were added CuI (893 mg, 4.7 mmol) and L-proline (1 g, 9.4 mmol). The mixture was stirred at 80° C. for 16 hours. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (PE:EA=50:1-30:1) to afford the product (4.9 g, 32.5%). [M+H]⁺=320.1.

### Step 2: methyl (R)-1-(4-(2,6-bis(benzyloxy)pyridin-3-yl)-3,5-difluorophenyl)pyrrolidine-3-carboxylate

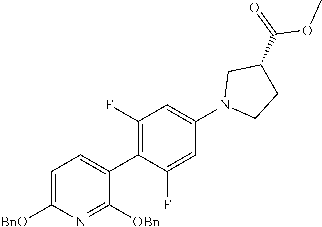 [SMILES: COC(=O)[C@@H]1CCN(c2cc(F)c(-c3ccc(OCc4ccccc4)nc3OCc3ccccc3)c(F)c2)C1]

To the solution of methyl (R)-1-(4-bromo-3,5-difluorophenyl)pyrrolidine-3-carboxylate (4.9 g, 15.3 mmol), 2,6-bis(benzyloxy)-3-(4,4,5,5-tetramethyl-1,3,2-dioxaborolan-2-yl)pyridine (6.7 g, 16 mmol) and CsF (4.6 g, 30.6 mmol) in 150 mL DMF and 15 mL water, was added Pd(dtbpf)Cl₂ (498 mg, 0.8 mmol). The mixture was stirred at 80° C. for 4 hours. After LCMS showed the reaction was completed, the mixture was diluted with water and extracted with EtOAc. The organic layer was washed with brine, dried over anhydrous Na₂SO₄, filtered and concentrated under reduced pressure. The resulting mixture was purified by combi-flash (EA:PE=0-12%) to afford the product (7.9 g, 97.4%). [M+H]⁺=531.30.

### Step 3: (R)-1-(4-(2,6-bis(benzyloxy)pyridin-3-yl)-3,5-difluorophenyl)pyrrolidine-3-carboxylic acid

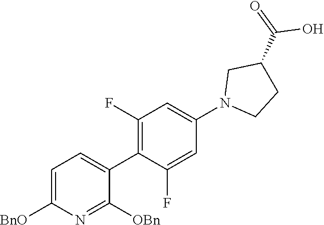 [SMILES: O=C(O)[C@@H]1CCN(c2cc(F)c(-c3ccc(OCc4ccccc4)nc3OCc3ccccc3)c(F)c2)C1]

To the solution of methyl (R)-1-(4-(2,6-bis(benzyloxy)pyridin-3-yl)-3,5-difluorophenyl)pyrrolidine-3-carboxylate (7.9 g, 14.9 mmol) in 100 mL THF and 20 mL water, LiOH (394 mg, 16.4 mmol) in 10 mL water was added dropwise at room temperature. The mixture was stirred at room temperature for 15 minutes. After TLC showed the reaction was completed, the mixture was concentrated in vacuum at room temperature. The residue was diluted with water and adjust pH<5 with 1 N HCl. The liquid was extracted with EtOAc. The organic phase was dried over anhydrous Na₂SO₄, filtered and concentrated in vacuum to afford the product (7.4 g, 96.0%). [M+H]⁺=517.1.

### Step 4: (R)-1-(4-((R)-2,6-dioxopiperidin-3-yl)-3,5-difluorophenyl)pyrrolidine-3-carboxylic

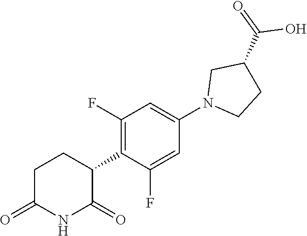 [SMILES: O=C1CC[C@H](c2c(F)cc(N3CC[C@@H](C(=O)O)C3)cc2F)C(=O)N1]

To the solution of (R)-1-(4-(2,6-bis(benzyloxy)pyridin-3-yl)-3,5-difluorophenyl)pyrrolidine-3-carboxylic acid (7.4 g, 14.3 mmol) in 50 mL DCM and 250 mL iPrOH, Pd/C (7.4 g, 10 wt. %, wet) was added. The mixture was stirred at 40° C. for 16 hours under hydrogen atmosphere (balloon). After LCMS showed the reaction was completed, the mixture was cooled to room temperature and filtered by celite directly. The filtrate was concentrated in vacuum to afford the crude product which was purified by SFC (IH (3*25 cm, Sum), 13% EtOH/87% CO₂, 100 bar, 100 ml/min) and the title compound corresponded to peak A @ 0.655 min/254 nm (1.6 g, 34%). [M+H]⁺=339.2.

### Step 5: tert-butyl (R,E)-4-((1¹,2⁶,7-trimethyl-3-oxo-5²,5³-dihydro-1¹H,5¹H-11-oxa-4-aza-5(2,1)-benzo[d]imidazola-2(2,4-pyridina-1(4,5)-pyrazolacycloundecaphane-5⁶-yl)methyl)piperazine-1-carboxylate

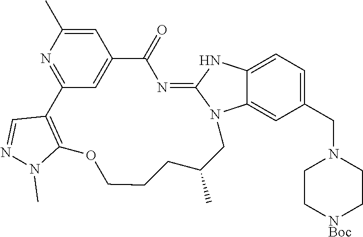 [SMILES: Cc1cc2cc(n1)-c1cnn(C)c1OCCC[C@@H](C)CN1/C(=N/C2=O)Nc2ccc(CN3CCN(C(=O)OC(C)(C)C)CC3)cc21]

To a 250-mL round-bottomed flask equipped with a magnetic stir bar was added (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (2.6 g, 5.5 mmol), tert-butyl piperazine-1-carboxylate (8.2 g, 44.0 mmol), NaBH(OAc)₃ (5.8 g, 27.5 mmol) and DCM (100 mL) at room temperature. To the stirred reaction mixture was added AcOH (1.9 mL, 33.0 mmol). After stirring for 12 h at room temperature, the reaction mixture was diluted with sat. aq. NaHCO₃ (150 mL) and the layers were separated. The aqueous layer was extracted with DCM (3×70 mL). The combined organic layers were washed with brine (150 mL), dried over anhydrous Na₂SO₄, filtered and concentrated under reduced pressure. Purification by flash column chromatography (0-10% MeOH in DCM) afforded the product (3.1 g, 9(W/o). [M+H]⁺=629.6.

### Step 6: (R,E)-1¹,2⁶,7-trimethyl-5⁶(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

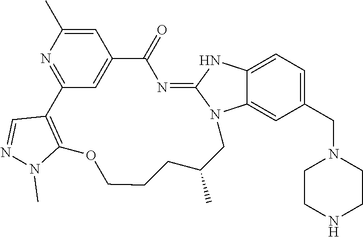 [SMILES: Cc1cc2cc(n1)-c1cnn(C)c1OCCC[C@@H](C)CN1/C(=N/C2=O)Nc2ccc(CN3CCNCC3)cc21]

To a 100-mL round-bottomed flask equipped with a magnetic stir bar was added tert-butyl (R,E)-4-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carboxylate (3.1 g, 5.0 mmol), DCM (40 mL) and TFA (10 mL). After stirring for 1 h at room temperature, the reaction mixture was concentrated under reduced pressure to afford the product (6.0 g), which was used without further purification. [M+H]⁺=529.4.

### Step 7: (R)-3-(2,6-difluoro-4-((R)-3-(4-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(45)-pyrazolacycloundecaphane-5⁶-yl)methyl)piperazine-1-carbonyl)pyrrolidin-1-yl)phenyl)piperidine-2,6-dione

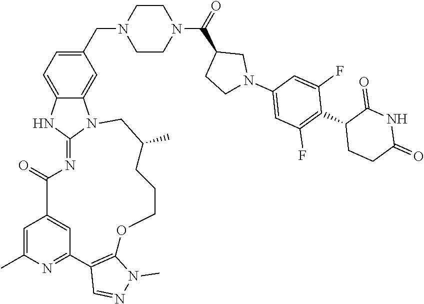 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21]

To a 250-mL round-bottomed flask equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (5.0 g), (R)-1-(4-((R)-2,6-dioxopiperidin-3-yl)-3,5-difluorophenyl)pyrrolidine-3-carboxylic acid (1.7 g, 5.0 mmol) and DCM (100 mL). To the reaction mixture was added N,N-diethylpropylethylamine (8.8 mL, 50 mmol) and T3P (50 wt %, 6.4 g, 10 mmol). After stirring for 1.5 h at room temperature, the reaction mixture was diluted with brine (150 mL) and the layers were separated. The aqueous layer was extracted with DCM (3×70 mL). The combined organic layers were washed with brine (150 mL), dried over anhydrous Na₂SO₄, filtered and concentrated under reduced pressure. Purification by flash column chromatography (0-6% MeOH in DCM) afforded the title compound (3.3 g, 78%). ¹H NMR (500 MHz, DMSO) δ 12.66 (s, 1H), 10.79 (s, 1H), 8.36 (s, 1H), 7.86 (s, 1H), 7.50 (s, 1H), 7.47 (s, 1H), 7.43 (d, J=8.0 Hz, 1H), 7.13 (d, J=8.5 Hz, 1H), 6.15 (d, J=12.0 Hz, 2H), 4.33-4.26 (m, 1H), 4.15-4.12 (m, 1H), 4.00-3.85 (m, 1H), 3.66 (s, 3H), 3.55-3.41 (m, 9H), 3.23-3.14 (m, 5H), 2.74-2.67 (m, 2H), 2.49 (s, 3H), 2.36-2.29 (m, 4H), 2.19-2.03 (m, 2H), 2.03-1.82 (m, 5H), 1.43-1.37 (m, 1H), 0.75 (d, J=6.5 Hz, 3H); [M+H]⁺=849.8.

### Example 6

### (R)-3-(2,6-difluoro-4-((R)-3-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carbonyl)pyrrolidin-1-yl)phenyl)piperidine-2,6-dione

### Step 1: tert-butyl (R,E)-4-(1-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carboxylate

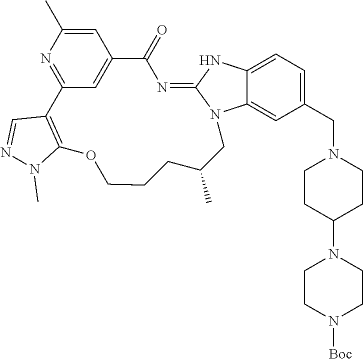 [SMILES: Cc1cc2cc(n1)-c1cnn(C)c1OCCC[C@@H](C)CN1/C(=N/C2=O)Nc2ccc(CN3CCC(N4CCN(C(=O)OC(C)(C)C)CC4)CC3)cc21]

To a 100-mL round-bottomed flask equipped with a magnetic stir bar was added (R,E)-1¹,2′,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (916 mg, 2.0 mmol), tert-butyl 4-(piperidin-4-yl)piperazine-1-carboxylate (5.4 g, 20.0 mmol), NaBH(OAc)₃ (1.3 g, 12.0 mmol) and DCM (40 mL) at room temperature. To the stirred reaction mixture was added AcOH (1.2 mL, 20.0 mmol). After stirring for 12 h at room temperature, the reaction mixture was diluted with sat. aq. NaHCO₃(50 mL) and the layers were separated. The aqueous layer was extracted with DCM (3×40 mL). The combined organic layers were washed with brine (50 mL), dried over anhydrous Na₂SO₄, filtered and concentrated under reduced pressure. Purification by flash column chromatography (0-10% MeOH in DCM) afforded the product (780 mg, 55%). [M+H]⁺=712.8.

### Step 2: (R,E)-1¹,2⁶,7-trimethyl-5⁶-((4-(piperazin-1-yl)piperidin-1-yl)methyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

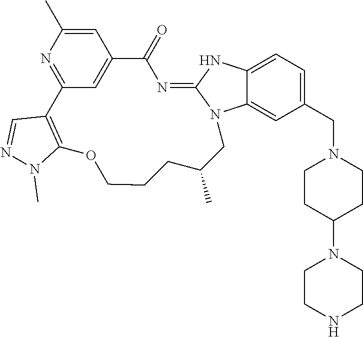 [SMILES: Cc1cc2cc(n1)-c1cnn(C)c1OCCC[C@@H](C)CN1/C(=N/C2=O)Nc2ccc(CN3CCC(N4CCNCC4)CC3)cc21]

To a 100-mL round-bottomed flask equipped with a magnetic stir bar was added tert-butyl (R,E)-4-(0-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carboxylate (780 mg, 1.1 mmol), DCM (30 mL) and TFA (7 mL). After stirring for 1 h at room temperature, the reaction mixture was concentrated under reduced pressure to afford the product (1.4 g), which was used without further purification. [M+H]⁺=612.5.

### Step 3: (R)-3-(2,6-difluoro-4-((R)-3-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carbonyl)pyrrolidin-1-yl)phenyl)piperidine-2,6-dione

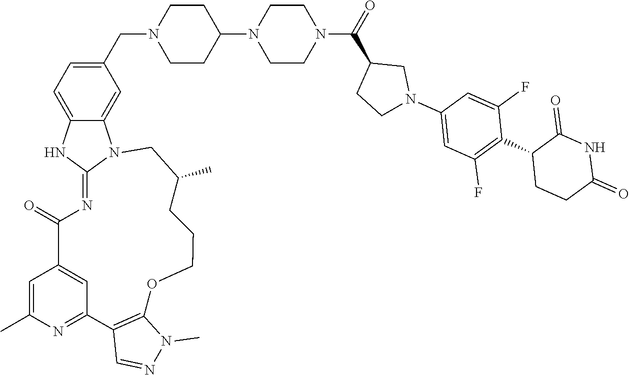 [SMILES: Cc1cc2cc(n1)-c1cnn(C)c1OCCC[C@@H](C)CN1/C(=N/C2=O)Nc2ccc(CN3CCC(N4CCN(C(=O)[C@@H]5CCN(c6cc(F)c([C@H]7CCC(=O)NC7=O)c(F)c6)C5)CC4)CC3)cc21]

To a 40-mL microwave vial equipped with a magnetic stir bar was added (R,E)-1¹,2⁶,7-trimethyl-5⁶-(4-(piperazin-1-yl)piperidin-1-yl)methyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (61.1 mg, 0.1 mmol), (R)-1-(4-((R)-2,6-dioxopiperidin-3-yl)-3,5-difluorophenyl)pyrrolidine-3-carboxylic acid (67.7 mg, 0.2 mmol) and DCM (8 mL). To the reaction mixture was added N,N-diethylpropylethylamine (340 mL, 2.0 mmol) and T3P (50 wt %, 380 mg, 0.6 mmol).

After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (52 mg, 56%). ¹H NMR (500 MHz, DMSO) δ 12.74 (s, 1H), 10.84 (s, 1H), 8.43 (s, 1H), 7.93 (s, 1H), 7.57 (s, 1H), 7.55-7.50 (m, 2H), 7.23-7.18 (m, 1H), 6.21 (d, J=12.0 Hz, 2H), 4.39-4.34 (m, 1H), 4.23-4.20 (m, 1H), 4.03-4.00 (m, 2H), 3.94-3.86 (m, 1H), 3.73 (s, 3H), 3.57-3.19 (m, 18H), 2.84-2.72 (m, 2H), 2.56 (s, 3H), 2.54-2.44 (m, 3H), 2.26-1.89 (m, 8H), 1.82-1.71 (m, 2H), 1.54-1.43 (m, 2H), 0.82 (d, J=6.5 Hz, 3H); [M+H]⁺=932.8.

### Example 7 (R)-3-(2,6-difluoro-4-(4-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)piperidin-1-yl)phenyl)piperidine-2,6-dione

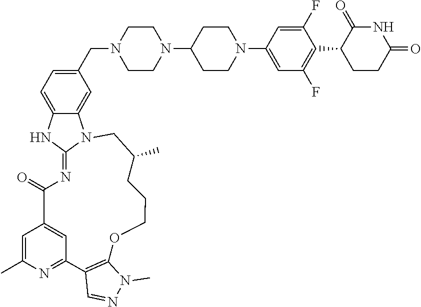 [SMILES: Cc1cc2cc(n1)-c1cnn(C)c1OCCC[C@@H](C)CN1/C(=N/C2=O)Nc2ccc(CN3CCN(C4CCN(c5cc(F)c([C@H]6CCC(=O)NC6=O)c(F)c5)CC4)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (158.6 mg, 0.3 mmol), (R)-3-(2,6-difluoro-4-(4-oxopiperidin-1-yl)phenyl)piperidine-2,6-dione (the compound was obtained through the similar way of example 5) (32.3 mg, 0.1 mmol) and DCE (8 mL). To the reaction mixture was added N,N-diethylpropylethylamine (0.5 mL, 3.0 mmol) and Ti(OiPr)₄ (0.3 mL, 1.0 mmol). After stirring for 12 h at 70° C., the reaction mixture was cooled down to room temperature and NaBH(OAc)₃ (84 mg, 0.4 mmol) was added. After stirring for 2 h at room temperature, the reaction mixture was concentrated under reduced pressure. Purification by flash column chromatography (0-70% MeOH in DCM) followed by prep-HPLC chromatography (0.1% FA in water:acetonitrile=90:10-50:50 gradient elution) afforded the title compound (38 mg, 45%). ¹H NMR (500 MHz, DMSO) δ 12.70 (s, 1H), 10.86 (s, 1H), 8.43 (s, 1H), 7.92 (s, 1H), 7.57 (s, 1H), 7.49-7.47 (m, 2H), 7.17 (d, J=7.5 Hz, 1H), 6.61 (d, J=13.0 Hz, 2H), 4.39-4.34 (m, 1H), 4.23-4.16 (m, 1H), 4.07-3.98 (m, 2H), 3.96-3.91 (m, 1H), 3.79-3.73 (m, 2H), 3.36-3.28 (m, 4H), 3.73 (s, 3H), 2.83-2.70 (m, 4H), 2.56 (s, 3H), 2.54-2.49 (m, 4H), 2.43-2.32 (m, 4H), 2.26-2.17 (m, 1H), 2.11-1.89 (m, 4H), 1.81-1.78 (m, 2H), 1.51-1.38 (m, 3H), 0.81 (d, J=6.5 Hz, 3H); [M+H]⁺=835.8.

### Example 8 (R)-3-(2,6-difluoro-4-(4-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazin-1-yl)piperidin-1-yl)phenyl)piperidine-2,6-dione

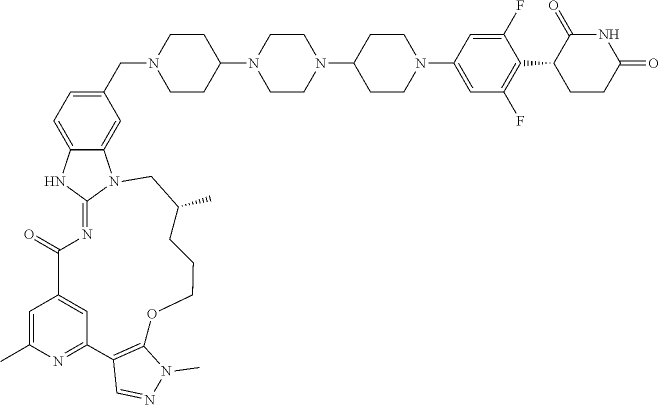 [SMILES: Cc1cc2cc(n1)-c1cnn(C)c1OCCC[C@@H](C)CN1/C(=N/C2=O)Nc2ccc(CN3CCC(N4CCN(C5CCN(c6cc(F)c([C@H]7CCC(=O)NC7=O)c(F)c6)CC5)CC4)CC3)cc21]

To a 40-mL microwave vial equipped with a magnetic stir bar was added (R,E)-11,26,7-trimethyl-56-((4-(piperazin-1-yl)piperidin-1-yl)methyl)-52,53-dihydro-11H,51H-11-oxa-4-aza-5(2,1)-benzo[d]imidazola-2(2,4)-pyridina-1(4,5)-pyrazolacycloundecaphan-3-one (183.3 mg, 0.3 mmol), (R)-3-(2,6-difluoro-4-(4-oxopiperidin-1-yl)phenyl)piperidine-2,6-dione (32.3 mg, 0.1 mmol) and DCE (8 mL). To the reaction mixture was added N,N-diethylpropylethylamine (0.5 mL, 3.0 mmol) and Ti(OiPr)₄ (0.3 mL, 1.0 mmol). After stirring for 12 h at 70° C., the reaction mixture was cooled down to room temperature and NaBH(OAc)₃ (84 mg, 0.4 mmol) was added. After stirring for 2 h at room temperature, the reaction mixture was concentrated under reduced pressure. Purification by flash column chromatography (0-70% MeOH in DCM) followed by prep-HPLC chromatography (0.1% FA in water:acetonitrile=90:10-50:50 gradient elution) afforded the title compound (44 mg, 48%).

¹H NMR (500 MHz, DMSO) δ 12.70 (s, 1H), 10.86 (s, 1H), 8.43 (s, 1H), 7.92 (s, 1H), 7.57 (s, 1H), 7.49-7.47 (m, 2H), 7.16 (d, J=8.5 Hz, 1H), 6.61 (d, J=13.0 Hz, 2H), 4.40-4.32 (m, 1H), 4.23-4.16 (m, 1H), 4.08-3.97 (m, 2H), 3.97-3.90 (m, 1H), 3.81-3.73 (m, 2H), 3.73 (s, 3H), 2.92-2.60 (m, 8H), 2.56 (s, 3H), 2.54-2.45 (m, 5H), 2.38-2.29 (m, 2H), 2.25-2.05 (m, 4H), 2.05-1.89 (m, 6H), 1.84-1.69 (m, 4H), 1.48-1.37 (m, 6H), 0.81 (d, J=6.5 Hz, 3H); [M+H]⁺=918.8.

### Example 9: (R)-3-(2,6-difluoro-4-((R)-3-(9-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-3,9-diazaspiro[5.5]undecane-3-carbonyl)pyrrolidin-1-yl)phenyl)piperidine-2,6-dione

### Step 1; methyl 2-(5-hydroxy-1-methyl-1H-pyrazol-4-yl)-6-methylisonicotinate

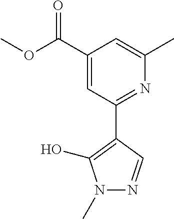

To a solution of compound methyl 2-chloro-6-methyl-pyridine-4-carboxylate (200 g, 1.08 mol) and 2-methylpyrazol-3-ol (211.42 g, 2.16 mol) in ANISOLE (3.5 L) was added Na₂CO₃ (285.52 g, 2.69 mol) and Pd(dppf)C₁₂·CH₂Cl₂ (26.40 g, 32.33 mmol). The mixture was stirred at 130° C. under N₂ for 12 hours and turned to brown suspension. The reaction mixture was then cooled to 20° C. and was filtered through a 2 cm pad of celite. The celite pad was washed with toluene (2 L). The dark filtrate was treated with MeOH (2 L) follow by dropwise addition of 4 M HCl in dioxane (1.2 L). The resultant slurry was stirred at 20° C. for 1 hour and then was filtered. The filter cake was washed with MTBE (2 L) and was then dried under vacuum at 50° C. to give a yellow solid. Compound methyl 2-(5-hydroxy-1-methyl-pyrazol-4-yl)-6-methyl-pyridine-4-carboxylate (334 g, 1.35 mol, 41.79% yield) was obtained. ¹H NMR (400 MHz, CHLOROFORM-d) δ 7.72 (d, J=0.6 Hz, 1H), 7.70 (s, 1H), 7.24 (s, 1H), 3.98 (s, 3H), 3.64 (s, 3H), 2.59 (s, 3H).

### Step 2: (R)-5-((5-bromo-2-nitrophenyl)amino)₄-methylpentyl methanesulfonate

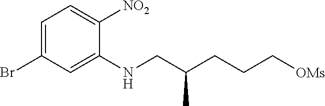 [SMILES: C[C@H](CCCOS(C)(=O)=O)CNc1cc(Br)ccc1[N+](=O)[O-]]

To a solution of (4R)-5-(5-bromo-2-nitro-anilino)-4-methyl-pentan-1-ol (200 g, 630.56 mmol) in DCM (1.6 L) was added TEA (95.71 g, 945.84 mmol, 131.65 mL) and MsCl (79.07 g, 690.26 mmol, 53.43 mL) at 0° C. The mixture was stirred at 15° C. for 12 hours. Then the reaction mixture was washed with saturated aqueous of NaHCO₃ (700 mL*3), and then the aqueous layers were combined and extracted with DCM (600 mL*3). The organic layers were combined and dried over Na₂SO₄, filtered and concentrated under reduced pressure to give a product (240 g, 582.29 mmol, 92.34% yield). The product was used next step without purification. [M+H]⁺=395.1.

### Step 3: methyl (R)-2-(5-((5-((5-bromo-2-nitrophenyl)amino)-4-methylpentyl)oxy)-1-methyl-1H-pyrazol-4-yl)-6-methylisonicotinate

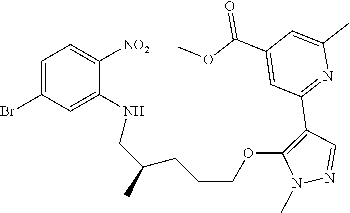 [SMILES: COC(=O)c1cc(C)nc(-c2cnn(C)c2OCCC[C@@H](C)CNc2cc(Br)ccc2[N+](=O)[O-])c1]

To a solution of (R)-5-((5-bromo-2-nitrophenyl)amino)-4-methylpentyl methanesulfonate (215.95 g, 546.33 mmol) and methyl 2-(5-hydroxy-1-methyl-pyrazol-4-yl)-6-methyl-pyridine-4-carboxylate (155 g, 546.33 mmol) in DMF (2 L) was added K₂CO₃ (151.01 g, 1.09 mol) at 15° C. The mixture was stirred at 60° C. for 12 hours and turned to yellow suspension. The reaction mixture was added water (500 mL), and then extracted with EtOAc (300 mL*3). The combined organic layers were washed with brine (500 mL), dried over Na₂SO₄, filtered and concentrated under reduced pressure to give a residue. The residue was purified by column chromatography (SiO₂, Petroleum ether/Ethyl acetate=20/1 to 0/1) to give product (185 g, 321.64 mmol, 58.87% yield, 95% purity). [M+H]⁺=546.2.

### Step 4: methyl (R)-2-(5-((5-((2-amino-5-bromophenyl)amino)-4-methylpentyl)oxy)-1-methyl-1H-pyrazol-4-yl)-6-methylisonicotinate

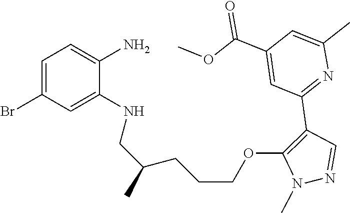 [SMILES: COC(=O)c1cc(C)nc(-c2cnn(C)c2OCCC[C@@H](C)CNc2cc(Br)ccc2N)c1]

A mixture of methyl (R)-2-(5-((5-((5-bromo-2-nitrophenyl)amino)-4-methylpentyl)oxy)-1-methyl-1H-pyrazol-4-yl)-6-methylisonicotinate (15.0 g, 26.6 mmol) and Pt/C (894 mg, 212 umol), in MeOH (225 mL) was degassed and purged with H₂(40 Psi) for 3 times at 20° C. and then the mixture was stirred at 30° C. for 24 hours under H₂ atmosphere and turned black suspension. The mixture was filtered and concentrated under reduced pressure. The product (14.0 g, 24.9 mmol, 93.6% yield) was used for next step without purification. [M+H]⁺=516.1.

### Step 5: methyl (R)-2-(5-((5-(2-amino-6-bromo-1H-benzo[d]imidazol-1-yl)-4-methylpentyl)oxy)-1-methyl-1H-pyrazol-4-yl)-6-methylisonicotinate

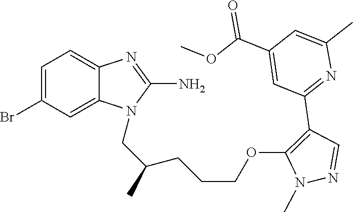 [SMILES: COC(=O)c1cc(C)nc(-c2cnn(C)c2OCCC[C@@H](C)Cn2c(N)nc3ccc(Br)cc32)c1]

To a solution of compound methyl (R)-2-(5-((5-((2-amino-5-bromophenyl)amino)-4-methylpentyl)oxy)-1-methyl-1H-pyrazol-4-yl)-6-m ethylisonicotinate (14.0 g, 24.9 mmol) in DCM (112 mL) and t-BuOH (23.8 mL) was added CNBr (5.89 g, 55.6 mmol, 4.09 mL) at 15° C. The mixture was stirred at 15° C. for 24 hours. Then the reaction mixture was quenched by aqueous NaHCO₃ solution (60 mL) for 10 minutes. The layers were separated and the organic layer was washed with saturated aqueous NaHCO₃ solution (60 mL*2). The organic layer was dried over Na₂SO₄, filtered and concentrated under reduced pressure to give a product (14.0 g, 24.4 mmol, 97.7% yield) which was used in the next step without further purification. [M+H]⁺=541.3.

### Step 6: (R)-2-(5-((5-(2-amino-6-bromo-1H-benzo[d]imidazol-1-yl)-4-methylpentyl)oxy)-1-methyl-1H-pyrazol-4-yl)-6-methylisonicotinic acid

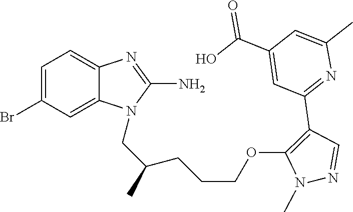 [SMILES: Cc1cc(C(=O)O)cc(-c2cnn(C)c2OCCC[C@@H](C)Cn2c(N)nc3ccc(Br)cc32)n1]

To a solution of methyl (R)-2-(5-((5-(2-amino-6-bromo-1H-benzo[d]imidazol-1-yl)-4-methylpentyl)oxy)-1-methyl-1H-pyrazol-4-yl)-6-methylisonicotinate (14.0 g, 25.8 mmol) in THF (112 mL) was added NaOH (4.14 g, 103 mmol) in H₂O (112 mL) at 15° C. The mixture was stirred at 15° C. for 1 hour. Then the reaction mixture was concentrated under reduced pressure to remove solvent. The residue was acidified with 4M aqueous HCl to pH=5, resulting in the formation of a slurry. The solid was filtered, washed with water (30 mL) and dried under reduced pressure to give a product. The product (14.0 g, crude) was used for next step without purification. [M−H]⁻=525.2.

### Step 7: (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

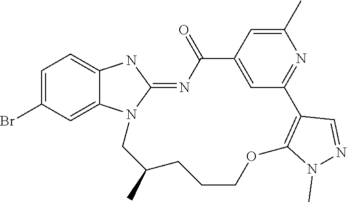 [SMILES: Cc1cc2cc(n1)-c1cnn(C)c1OCCC[C@@H](C)CN1/C(=N/C2=O)Nc2ccc(Br)cc21]

To a solution of (R)-2-(5-((5-(2-amino-6-bromo-1H-benzo[d]imidazol-1-yl)-4-methylpentyl)oxy)-1-methyl-1H-pyrazol-4-yl)-6-methylisonicotinic acid (13.0 g, 24.6 mmol) in DCM (210 mL) was added TBTU (9.50 g, 29.5 mmol) and TEA (3.74 g, 36.9 mmol, 5.15 mL) at 20° C. The mixture was stirred at 20° C. for 1 hour and turned black solution. The reaction mixture was washed with saturated NaHCO₃ solution (100 mL*3), dried over Na₂SO₄, filtered and concentrated under reduced pressure to give a residue. The residue was purified by column chromatography (SiO₂, Dichloromethane:Methanol=100/1 to 10/1) to afford the product (10.3 g, 82.0% yield). ¹H NMR (400 MHz, DMSO): δ 12.79 (br s, 1H), 8.36 (s, 1H), 7.83-7.92 (m, 2H), 7.51 (s, 1H), 7.39-7.46 (m, 1H), 7.30-7.38 (m, 1H), 4.22-4.37 (m, 1H), 4.10 (dd, J=13.59, 2.85 Hz, 1H), 3.89-3.98 (m, 2H), 3.68 (s, 3H), 2.68-2.82 (m, 1H), 2.51 (s, 3H), 2.09-2.23 (m, 1H), 1.78-1.98 (m, 2H), 1.33-1.45 (m, 1H), 0.76 (d, J=6.58 Hz, 3H).

### Step 8: (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde

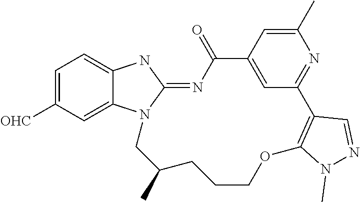 [SMILES: Cc1cc2cc(n1)-c1cnn(C)c1OCCC[C@@H](C)CN1/C(=N/C2=O)Nc2ccc(C=O)cc21]

To a solution of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)pyrazolacycloundecaphan-3-one (10.3 g, 20.2 mmol) in dioxane (16 mL) was added Pd(OAc)₂ (453 mg, 2.02 mmol) and bis(1-adamantyl)-butyl-phosphane (2.17 g, 6.07 mmol) and TMEDA (4.70 g, 40.4 mmol, 6.10 mL) at 20° C. The suspension was degassed under vacuum and purged with CO/H₂ several times. The mixture was stirred at 100° C. for 48 hours. Then the reaction mixture was filtered and concentrated under reduced pressure to give a residue. The residue was purified by column chromatography (SiO₂, Petroleum ether/Ethyl acetate=10/1 to 0/1) to afford the product (4.50 g, 48.5% yield).

¹H NMR: (400 MHz, CDCl₃): δ 10.04 (s, 1H), 8.46 (s, 1H), 8.16 (s, 1H), 7.82 (s, 1H), 7.76 (br d, J=6.91 Hz, 1H), 7.65 (s, 1H), 7.47 (br d, J=7.75 Hz, 1H), 4.30-4.52 (m, 2H), 3.86-3.97 (m, 1H), 3.80 (s, 3H), 2.86 (br s, 1H), 2.60 (s, 3H), 2.21-2.35 (m, 1H), 2.14 (ddd, J=13.83, 9.18, 4.53 Hz, 1H), 1.95 (br s, 2H), 1.45-1.73 (m, 2H), 0.93 (d, J=6.56 Hz, 3H). [M+H]⁺=459.3.

### Step 9: tert-butyl (R,E)-9-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecane-3-carboxylate

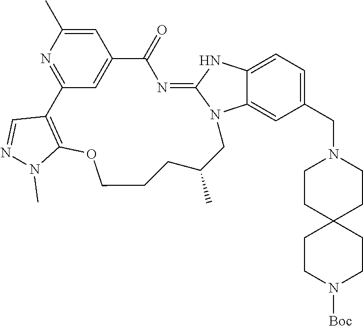 [SMILES: Cc1cc2cc(n1)-c1cnn(C)c1OCCC[C@@H](C)CN1/C(=N/C2=O)Nc2ccc(CN3CCC4(CC3)CCN(C(=O)OC(C)(C)C)CC4)cc21]

To a 100-mL round-bottomed flask equipped with a magnetic stir bar was added (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (458 mg, 1.0 mmol), tert-butyl 3,9-diazaspiro[5.5]undecane-3-carboxylate (1.01 g, 4.0 mmol), NaBH(OAc)₃ (636 mg, 3.0 mmol) and DCM (30 mL) at room temperature. To the stirred reaction mixture was added AcOH (457 mL, 8.0 mmol). After stirring for 12 h at room temperature, the reaction mixture was diluted with sat. aq. NaHCO₃(30 mL) and the layers were separated. The aqueous layer was extracted with DCM (3×30 mL). The combined organic layers were washed with brine (50 mL), dried over anhydrous Na₂SO₄, filtered and concentrated under reduced pressure. Purification by flash column chromatography (0-10% MeOH in DCM) afforded the product (368 mg, 53%). [M+H]⁺=697.7.

### Step 10: (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

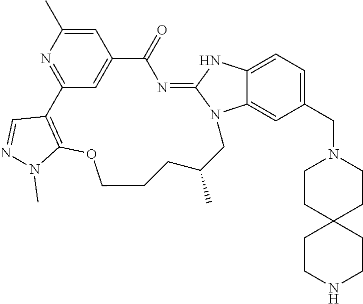 [SMILES: Cc1cc2cc(n1)-c1cnn(C)c1OCCC[C@@H](C)CN1/C(=N/C2=O)Nc2ccc(CN3CCC4(CCNCC4)CC3)cc21]

To a 100-mL round-bottomed flask equipped with a magnetic stir bar was added tert-butyl (R,E)-9-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecane-3-carboxy late (368 mg, 0.53 mmol), DCM (20 mL) and TFA (5 mL). After stirring for 1 h at room temperature, the reaction mixture was concentrated under reduced pressure to afford the product as TFA salt (720 mg), which was used without further purification. [M+H]⁺=597.5.

### Step 11: (R)-3-(2,6-difluoro-4-((R)-3-(9-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-3,9-diazaspiro[5,5]undecane-3-carbonyl)pyrrolidin-1-yl)phenyl)piperidine-2,6-dione

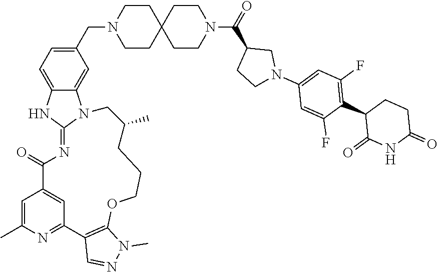 [SMILES: Cc1cc2cc(n1)-c1cnn(C)c1OCCC[C@@H](C)CN1/C(=N/C2=O)Nc2ccc(CN3CCC4(CC3)CCN(C(=O)[C@@H]3CCN(c5cc(F)c([C@H]6CCC(=O)NC6=O)c(F)c5)C3)CC4)cc21]

To a 40-mL microwave vial equipped with a magnetic stir bar was added (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (90.0 mg, 0.15 mmol), (R)-1-(4-((R)-2,6-dioxopiperidin-3-yl)-3,5-difluorophenyl)pyrrolidine-3-carboxylic acid (60.8 mg, 0.18 mmol) and DCM (8 mL). To the reaction mixture was added N,N-diethylpropylethylamine (260 mL, 1.5 mmol) and T3P (50 wt %, 286 mg, 0.45 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10˜50:50 gradient elution) afforded the title compound (42 mg, 31%). ¹H NMR (500 MHz, DMSO) δ 12.72 (s, 1H), 10.88 (s, 1H), 8.43 (s, 1H), 7.92 (s, 1H), 7.57 (s, 1H), 7.50 (s, 1H), 7.47 (d, J=8.0 Hz, 1H), 7.17 (d, J=8.0 Hz, 1H), 6.16 (d, J=11.0 Hz, 2H), 4.40-4.36 (m, 1H), 4.23-4.17 (m, 1H), 4.06-3.96 (m, 6H), 3.82-3.77 (m, 3H), 3.73 (s, 3H), 3.58-3.42 (m, 6H), 2.83-2.72 (m, 5H), 2.56 (s, 3H), 2.41-2.31 (m, 2H), 2.11-1.89 (m, 5H), 1.52-1.35 (m, 10H), 0.81 (d, J=6.5 Hz, 3H); [M+H]⁺=917.8.

### Example 10: (R)-3-(2,6-difluoro-4-(3-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carbonyl)azetidin-1-yl)phenyl)piperidine-2,6-dione

### Step 1: methyl 1-(4-(2,6-bis(benzyloxy)pyridin-3-yl)-3,5-difluorophenyl)azetidine-3-carboxylate

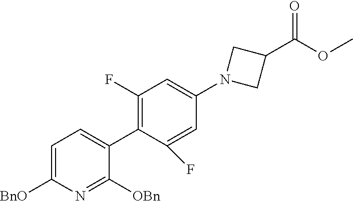 [SMILES: COC(=O)C1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (3.00 g, 6.22 mmol), methyl azetidine-3-carboxylate hydrochloride (1.41 g, 9.33 mmol), Cs₂CO₃ (6.06 g, 18.66 mmol) and RuPhos Pd G3 (520.7 mg, 0.622 mmol) in toluene (50 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+1]⁺=517.1.

### Step 2: 1-(4-(2,6-bis(benzyloxy)pyridin-3-yl)-3,5-difluorophenyl)azetidine-3-carboxylic acid

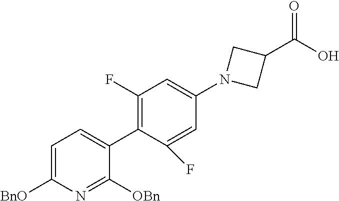 [SMILES: O=C(O)C1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiOH H₂O (168 mg, 4 mmol in 10 mL water) dropwise at room temperature. Then the mixture was stirred for 2 hours. The resulting mixture was concentrated in vacuum. The water layer was adjusted pH<5 with IN HCl and then extracted with EtOAc (3×50 mL). The combined organic layers were washed with brine, dried over anhydrous Na₂SO₄. After filtration, the filtrate was concentrated under reduced pressure to afford the product (1.4 g, 85%) [M+1]⁺=503.2.

### Step 3: (R)-1-(4-(2,6-dioxopiperidin-3-yl)-3,5-difluorophenyl)azetidine-3-carboxylic acid

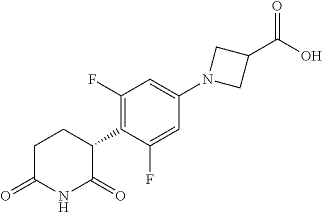 [SMILES: O=C1CC[C@H](c2c(F)cc(N3CC(C(=O)O)C3)cc2F)C(=O)N1]

To a solution of 1-(4-(2,6-bis(benzyloxy)pyridin-3-yl)-3,5-difluorophenyl)azetidine-3-carboxylic acid (1.40 g, 2.79 mmol) in iPrOH (20 mL) and DCM (20 mL) was added Pd/C (1.0 g, 10% wt) which was stirred at room temperature under hydrogen atmosphere for 48 hours. The resulting mixture was filtered, the filter cake was washed with MeOH (20 mL). The filtrate was concentrated under reduced pressure to afford the crude product. The residue was purified by SFC (IH (3*25 cm, Sum), 13% EtOH/87% CO₂, 100 bar, 100 ml/min) and the title compound corresponded to peak A @ 1.853 min/254 nm (190 mg, 21%). [M+1]⁺=325.3.

### Step 4: (R)-3-(2,6-difluoro-4-(3-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazine-1-carbonyl)azetidin-1-yl)phenyl)piperidine-2,6-dione

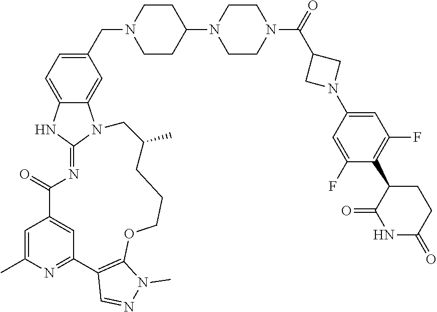 [SMILES: Cc1cc2cc(n1)-c1cnn(C)c1OCCC[C@@H](C)CN1/C(=N/C2=O)Nc2ccc(CN3CCC(N4CCN(C(=O)C5CN(c6cc(F)c([C@H]7CCC(=O)NC7=O)c(F)c6)C5)CC4)CC3)cc21]

To a 40-mL microwave vial equipped with a magnetic stir bar was added (R,E)-1¹,2⁶,7-trimethyl-5⁶-((4-(piperazin-1-yl)piperidin-1-yl)methyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (92.0 mg, 0.15 mmol), (R)-1-(4-(2,6-dioxopiperidin-3-yl)-3,5-difluorophenyl)azetidine-3-carboxylic acid (the compound was obtained through the similar way of example 5) (97.3 mg, 0.30 mmol) and DCM (8 mL). To the reaction mixture was added N,N-diethylpropylethylamine (520 mL, 3.0 mmol) and T3P (50 wt %, 570 mg, 0.90 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (102 mg, 74%). ¹H NMR (500 MHz, DMSO) δ 12.71 (s, 1H), 10.88 (s, 1H)), 8.43 (s, 1H), 7.92 (s, 1H), 7.57 (s, 1H), 7.49 (s, 1H), 7.47 (d, J=8.0 Hz, 1H), 7.16 (d, J=8.0 Hz, 1H), 6.16 (d, J=11.0 Hz, 2H), 4.39-4.34 (m, 1H), 4.23-4.15 (m, 1H), 4.05-3.89 (m, 8H), 3.82-3.77 (m, 2H), 3.73 (s, 3H), 3.59-3.42 (m, 3H), 2.91-2.70 (m, 5H), 2.56 (s, 3H), 2.50-2.40 (m, 5H), 2.27-2.16 (m, 2H), 2.08-1.86 (m, 6H), 1.71-1.69 (m, 2H), 1.51-1.35 (m, 3H), 0.80 (d, J=6.5 Hz, 3H); [M+H]⁺=918.4.

### Example 12: (R)-3-(2,6-difluoro-4-(3-(9-(((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecane-3-carbonyl)azetidin-1-yl)phenyl)piperidine-2,6-dione

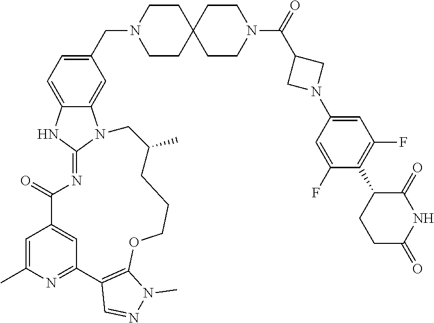 [SMILES: Cc1cc2cc(n1)-c1cnn(C)c1OCCC[C@@H](C)CN1/C(=N/C2=O)Nc2ccc(CN3CCC4(CC3)CCN(C(=O)C3CN(c5cc(F)c([C@H]6CCC(=O)NC6=O)c(F)c5)C3)CC4)cc21]

To a 40-mL microwave vial equipped with a magnetic stir bar was added (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (90.0 mg, 0.15 mmol), (R)-1-(4-(2,6-dioxopiperidin-3-yl)-3,5-difluorophenyl)azetidine-3-carboxylic acid (58.3 mg, 0.18 mmol) and DCM (8 mL). To the reaction mixture was added N,N-diethylpropylethylamine (260 mL, 1.5 mmol) and T3P (50 wt %, 286 mg, 0.45 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10˜ 50:50 gradient elution) afforded the title compound (51 mg, 38%). ¹H NMR (500 MHz, DMSO) δ 12.73 (s, 1H), 10.88 (s, 1H), 8.43 (s, 1H), 7.92 (s, 1H), 7.57 (s, 1H), 7.50 (s, 1H), 7.47 (d, J=8.5 Hz, 1H), 7.17 (d, J=8.0 Hz, 1H), 6.16 (d, J=11.0 Hz, 2H), 4.39-4.34 (m, 1H), 4.22-4.17 (m, 1H), 4.06-3.96 (m, 4H), 3.95-3.85 (m, 4H), 3.82-3.77 (m, 1H), 3.73 (s, 3H), 3.61-3.54 (m, 2H), 3.48-3.42 (m, 4H), 2.83-2.72 (m, 5H), 2.56 (s, 3H), 2.41-2.31 (m, 2H), 2.24-2.17 (m, 1H), 2.11-1.89 (m, 4H), 1.52-1.35 (m, 10H), 0.81 (d, J=6.5 Hz, 3H); [M+H]⁺=903.3.

### Example 14: (R)-3-(2,6-difluoro-4-(2-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)ethyl)phenyl)piperidine-2,6-dione

### Step 1: ethyl 4-(4-bromo-2,6-difluorophenyl)-4-cyanobutanoate

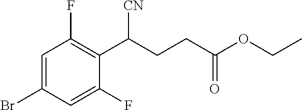 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2 M in THF, 24 mL, 48 mmol) dropwise in 20 min at −65° C., the reaction solution was stirred for 1 hour at this temperature, then to this was added ethyl 3-bromopropanoate (9.4 g, 51.7 mmol) in THF (30 mL) dropwise in 10 min. The resulting solution was stirred for 30 min at −65° C., and then allowed to warm to room temperature naturally. The reaction was quenched by the addition of sat. aq. NH₄Cl (50 mL), and extracted with EtOAc (100 mL×3). The combined organic layers were washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

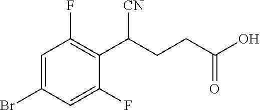 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and extracted with EtOAc (50 mL×2). The pH value of water phase was adjusted to 4-5 with 1 N HCl (10 mL), and extracted with EtOAc (50 mL×3). The combined organic layers were washed with brine (50 mL×3), and dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

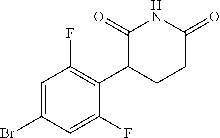 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added conc. H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃ (40 mL), and resulting solution was extracted with EtOAc (50 mL×3). The combined organic layers were washed with water and brine, dried over anhydrous Na₂SO₄, filtered and concentrated to afford the product (8.2 g, 80.4%). [M+H]⁺=304.3.

### Step 4: (R,E)-3-(4-(2-ethoxyvinyl)-2,6-difluorophenyl)piperidine-2,6-dione

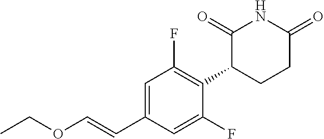 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF (8.2 g, 54.0 mmol). The resulting mixture was stirred for 2 h at 80° C. under nitrogen atmosphere. The reaction solution was diluted with water, extracted with EtOAc (100 mL×2). The combined organic layers were washed with water and brine, dried over anhydrous Na₂SO₄, filtered and concentrated under reduced pressure. The residue was purified by SFC (IH (3*25 cm, 5 um), 13% EtOH/87% CO₂, 100 bar, 100 ml/min) and the title compound corresponded to peak A @ 1.679 min/254 nm. (3.1 g, 39.0%). [M+H]⁺=296.1.

### Step 5; (R)-2-(4-(2,6-dioxopiperidin-3-yl)-3,5-difluorophenyl)acetaldehyde

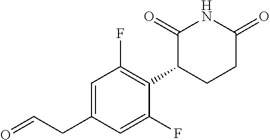 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### Step 6: (R)-3-(2,6-difluoro-4-(2-(4-(((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)ethyl)phenyl)piperidine-2,6-dione

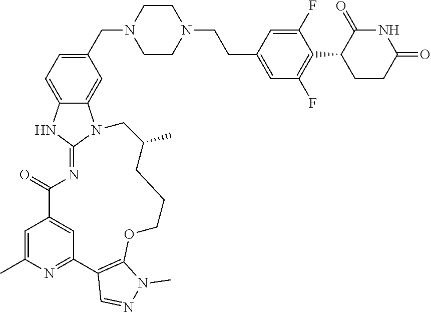 [SMILES: Cc1cc2cc(n1)-c1cnn(C)c1OCCC[C@@H](C)CN1/C(=N/C2=O)Nc2ccc(CN3CCN(CCc4cc(F)c([C@H]5CCC(=O)NC5=O)c(F)c4)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (52.9 mg, 0.1 mmol), (R)-2-(4-(2,6-dioxopiperidin-3-yl)-3,5-difluorophenyl)acetaldehyde (54.0 mg, 0.2 mmol), NaBH(OAc)₃ (63.6 mg, 0.3 mmol) and DCM (8 mL). After stirring for 12 h at 40° C., the reaction mixture was concentrated under reduced pressure. Purification by flash column chromatography (0-70% MeOH in DCM) followed by prep-HPLC chromatography (0.1% FA in water:acetonitrile=90:10˜50:50 gradient elution) afforded the title compound (43 mg, 55%). ¹H NMR (500 MHz, DMSO) δ 12.65 (s, 1H), 10.90 (s, 1H), 8.36 (s, 1H), 7.86 (s, 1H), 7.50 (s, 1H), 7.44 (s, 1H), 7.41 (d, J=8.0 Hz, 1H), 7.11 (d, J=8.0 Hz, 1H), 6.95 (d, J=10.5 Hz, 2H), 4.31-4.25 (m, 1H), 4.20-4.09 (m, 3H), 3.97-3.84 (m, 2H), 3.67 (s, 3H), 3.54-3.43 (m, 2H), 2.80-2.64 (m, 6H), 2.49 (s, 3H), 2.49-2.44 (m, 5H), 2.19-1.99 (m, 4H), 1.98-1.79 (m, 4H), 1.44-1.35 (m, 1H), 0.74 (d, J=6.0 Hz, 3H); [M+H]⁺=780.7.

### Example 1 3-(4-(2-(4-((R,E)-1′,2′,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁴-yl)piperidin-1-yl)ethyl)phenyl)piperidine-2,6-dione

### Step 1: 2-(4-(4,4,5,5-tetramethyl-1,3,2-dioxaborolan-2-yl)phenyl)ethan-1-ol

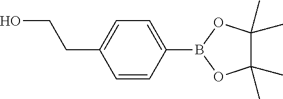 [SMILES: CC1(C)OB(c2ccc(CCO)cc2)OC1(C)C]

2-(4-bromophenyl)ethan-1-ol (20 g, 100 mmol), 4,4,4′,4′,5,5,5′,5′-Octamethyl-2,2′-bi-1,3,2-dioxaborolane (38.1 g, 150 mmol), Pd(dppf)Cl₂ (7.3 g, 10 mmol), KOAc (19.6 g, 200 mmol) were placed in dioxane (400 mL). The resulting mixture was then heated to reflux for 2 h. The mixture was cooled to room temperature, filtered off the solid and concentrated to afford crude product (28 g. crude), which was used directly without further purification. [M+H]*=249.2.

### Step 2: 2-(4-(2,6-bis(benzyloxy)pyridin-3-yl)phenyl)ethan-1-ol

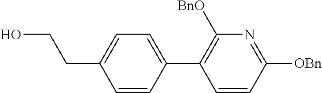 [SMILES: OCCc1ccc(-c2ccc(OCc3ccccc3)nc2OCc2ccccc2)cc1]

2-(4-(4,4,5,5-tetramethyl-1,3,2-dioxaborolan-2-yl)phenyl)ethan-1-ol (28 g, crude), Pd(dppf)Cl₂ (7.3 g, 10 mmol), 2,6-bis(benzyloxy)-3-bromopyridine (36.9 g, 10 mmol), Cs₂CO₃ (65.2 g, 200 mmol) were placed in Dioxane/water (300 mL, 10:1). The mixture was stirred at 100° C. overnight. Cooling the reaction to room temperature, filtered off the solid, the filtrate was concentrated and purified with SiO₂-gel column (eluted with EtOAc/Hexane=1:2) to give the crude product which was used directly in the next step. [M+H]⁺=412.2.

### Step 3: 3-(4(2-hydroxyethyl)phenyl)piperidine-2,6-dione

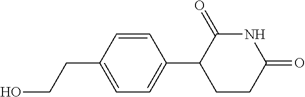 [SMILES: O=C1CCC(c2ccc(CCO)cc2)C(=O)N1]

2-(4-(2,6-bis(benzyloxy)pyridin-3-yl)phenyl)ethan-1-ol (the crude from last step) was dissolved in MeOH (500 mL), Pd/C (10%, w/w, 5 g) was added to the solution in one portion. The resulting mixture was stirred under H₂ atmosphere (1 atm) for overnight. Filtered off the solid, the filtrate concentrated to give the crude product. The crude was triturated with MTBE (50 mL) to give desire product (13.5 g, 57.9% over 3 steps). [M+H]⁺=234.1.

### Step 4: 2-(4-(2,6-dioxopiperidin-3-yl)phenyl)acetaldehyde

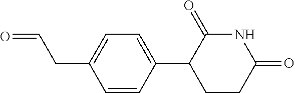 [SMILES: O=CCc1ccc(C2CCC(=O)NC2=O)cc1]

A mixture of 3-(4-(2-hydroxyethyl)phenyl)piperidine-2,6-dione (100 mg, 0.43 mmol) and IBX (132 mg, 0.47 mmol) in DMSO (10 mL) was stirred in a flask at room temperature overnight. The reaction was quenched with water and the mixture was extracted with EtOAc, washed three times with saturated aqueous NaCl and twice with saturated aqueous NaHCO₃. The organic layer was dried over anhydrous Na₂SO₄ and evaporated in vacuum to afford the product (70 mg, 70%). [M+H]⁺=232.19.

### Step 5: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-3,6-dihydropyridine-1(2H)-carboxylate

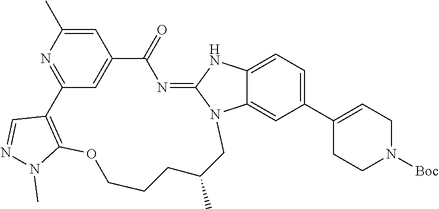 [SMILES: Cc1cc2cc(n1)-c1cnn(C)c1OCCC[C@@H](C)CN1/C(=N/C2=O)Nc2ccc(C3=CCN(C(=O)OC(C)(C)C)CC3)cc21]

To a mixture of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (200 mg, 0.39 mmol), tert-butyl 4-(4,4,5,5-tetramethyl-1,3,2-dioxaborolan-2-yl)-3,6-dihydropyridine-1(2H)-carboxylate (157 mg, 0.51 mmol), Pd(dppf)Cl₂ (29 mg, 0.04 mmol), Na₂CO₃ (83 mg, 0.78 mmol) in dioxane (10 mL) and H₂O (2 mL) was stirred at 90° C. under N₂ for 18 hrs. After cooling to rt, the reaction mixture was extracted with DCM (20 mL×3). The combined organic phase was washed with brine (20 mL), dried over Na₂SO₄, filtered and concentrated in vacuum. The residue was purified by silica gel column (DCM:CH₃OH=0 to 6:1) to afford tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-3,6-dihydropyridine-1(2H)-carboxylate (150 mg, 62.7%). [M+H]⁺=612.6.

### Step 6: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carboxylate

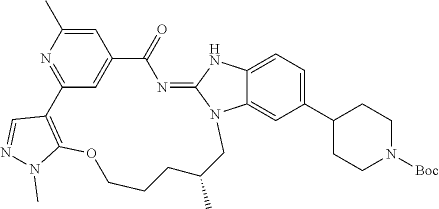 [SMILES: Cc1cc2cc(n1)-c1cnn(C)c1OCCC[C@@H](C)CN1/C(=N/C2=O)Nc2ccc(C3CCN(C(=O)OC(C)(C)C)CC3)cc21]

To a solution of tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-3,6-dihydropyridine-1(2H)-carboxylate (150 mg, 0.25 mmol) in THF (8 mL) was added Pd/C (90 mg, 10%). The reaction mixture was stirred for 4 hrs at r.t under H₂ balloon. The catalyst was filtered off, the filtrate was concentrated in vacuum to afford tert-butyl (R,E)-4-(1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carboxylate (140 mg, 93%). [M+H]⁺=614.6.

### Step 7: (R,E)-1¹,2⁶,7-trimethyl-5⁶-(piperidin-4-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

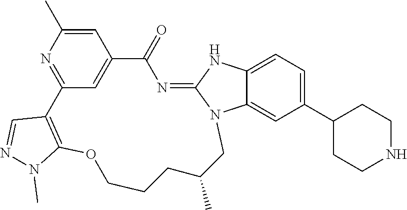 [SMILES: Cc1cc2cc(n1)-c1cnn(C)c1OCCC[C@@H](C)CN1/C(=N/C2=O)Nc2ccc(C3CCNCC3)cc21]

To a solution of tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carboxylate (140 mg, 0.23 mmol) in DCM (8 mL) was added TFA (2 mL). The reaction solution was stirred at r.t for 1 hrs and then concentrated in vacuo. The residue was dissolved in DCM and then washed with sat NaHCO₃ solution (5 mL) and brine (5 mL), dried over Na₂SO₄, filtered and concentrated in vacuum to afford (R,E)-1¹,2⁶,7-trimethyl-5⁶-(piperidin-4-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (80 mg, 68%). [M+H]⁺=514.6.

### Step 8: 3-(4-(2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-1-yl)ethyl)phenyl)piperidine-2,6-dione

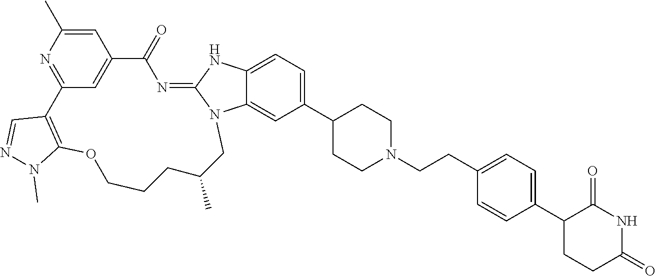 [SMILES: Cc1cc2cc(n1)-c1cnn(C)c1OCCC[C@@H](C)CN1/C(=N/C2=O)Nc2ccc(C3CCN(CCc4ccc(C5CCC(=O)NC5=O)cc4)CC3)cc21]

The title compound (12 mg, 27%) was prepared in a manner similar to that in Example 14 step 6 from (R,E)-1¹,2⁶,7-trimethyl-5⁶-(piperidin-4-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one and 2-(4-(2,6-dioxopiperidin-3-yl)phenyl)acetaldehyde. ¹H NMR (500 MHz, DMSO) δ 12.75-12.50 (m, 1H), 10.89 (s, 1H), 8.48-8.38 (m, 1H), 8.01-7.85 (m, 1H), 7.55 (d, J=10.1 Hz, 2H), 7.47-7.39 (m, 1H), 7.23-7.18 (m, 2H), 7.17-7.03 (m, 3H), 4.38-4.34 (m, 1H), 4.17 (d, J=11.2 Hz, 1H), 4.02-3.98 (m, 2H), 3.88-3.78 (m, 1H), 3.73 (s, 3H), 3.12 (d, J=10.3 Hz, 2H), 2.85-2.73 (m, 3H), 2.71-2.59 (m, 4H), 2.55 (d, J=6.5 Hz, 3H), 2.25-2.18 (m, 5H), 2.06-1.89 (m, 3H), 1.77 (d, J=27.3 Hz, 4H), 1.45 (d, J=6.2 Hz, 1H), 0.82 (d, J=6.4 Hz, 3H). [M+H]⁺=729.6.

### Example 2: 3-(2-fluoro-4-(4-(2-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)ethyl)piperidin-1-yl)phenyl)piperidine-2,6-dione

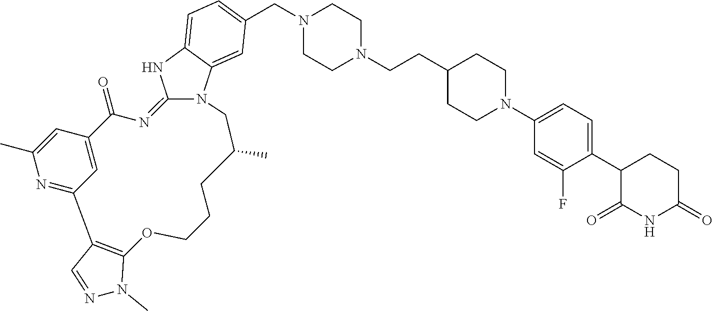 [SMILES: Cc1cc2cc(n1)-c1cnn(C)c1OCCC[C@@H](C)CN1/C(=N/C2=O)Nc2ccc(CN3CCN(CCC4CCN(c5ccc(C6CCC(=O)NC6=O)c(F)c5)CC4)CC3)cc21]

The titled compound was prepared in a manner similar to that in Example 14.

¹H NMR (500 MHz, DMSO) δ 12.71 (s, 1H), 10.80 (s, 1H), 8.43 (s, 1H), 7.92 (s, 1H), 7.57 (s, 1H), 7.55-7.39 (m, 2H), 7.17 (d, J=8.1 Hz, 1H), 7.05 (t, J=8.7 Hz, 1H), 6.82-6.51 (m, 2H), 4.36 (s, 1H), 4.20 (d, J=12.0 Hz, 1H), 3.96-3.85 (m, 3H), 3.73 (s, 3H), 3.68 (d, J=12.3 Hz, 2H), 3.57 (s, 2H), 2.79 (s, 1H), 2.72-2.59 (m, 4H), 2.55 (d, J=7.8 Hz, 4H), 2.43-2.36 (m, 8H), 2.21 (s, 1H), 2.15-2.05 (m, 1H), 2.03-1.86 (m, 4H), 1.72 (d, J=12.1 Hz, 2H), 1.54-1.34 (m, 4H), 1.27-1.14 (m, 2H), 0.81 (d, J=6.3 Hz, 3H). [M+H]⁺=845.6.

### Example 3: 3-(2,6-difluoro-4-(4-(2-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazin-1-yl)ethyl)piperidin-1-yl)phenyl)piperidine-2,6-dione

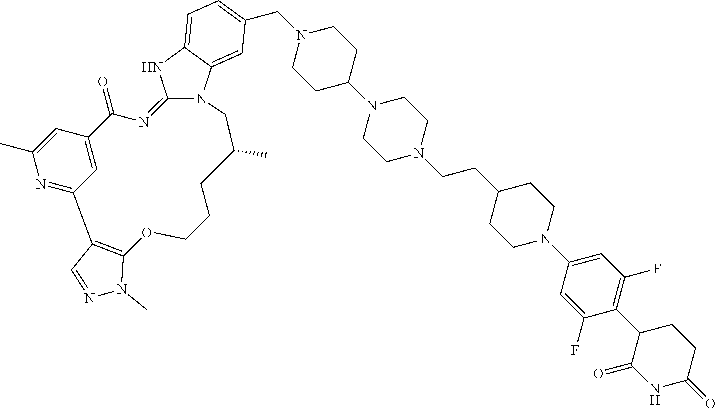 [SMILES: Cc1cc2cc(n1)-c1cnn(C)c1OCCC[C@@H](C)CN1/C(=N/C2=O)Nc2ccc(CN3CCC(N4CCN(CCC5CCN(c6cc(F)c(C7CCC(=O)NC7=O)c(F)c6)CC5)CC4)CC3)cc21]

The titled compound was prepared in a manner similar to that in Example 14. ¹H NMR (500 MHz, DMSO) δ 12.69 (s 1H), 10.86 (s, 1H), 8.43 (s, 1H), 7.92 (s, 1H), 7.57 (s, 1H), 7.51-7.40 (m, 2H), 7.15 (d, J=8.2 Hz, 1H), 6.59 (d, J=12.8 Hz, 2H), 4.39-4.32 (m, 1H), 4.19 (d, J=11.2 Hz, 1H), 4.10-3.86 (m, 3H), 3.73 (s, 3H), 3.71 (s, 2H), 3.53 (s, 2H), 2.85-2.73 (m, 4H), 2.72-2.65 (m, 3H), 2.56 (s, 5H), 2.48-2.40 (m, 3H), 2.38-2.25 (m, 5H), 2.22 (s, 1H), 2.15-2.05 (m, 2H), 1.96-1.90 (m, 5H), 1.74-1.68 (m, 4H), 1.53-1.30 (m, 6H), 1.16 (s, 2H), 0.81 (d, J=6.5 Hz, 3H). [M+H]⁺=946.8.

### Example 15: 3-(3-methyl-2-oxo-4-(4-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-yl)piperidin-1-yl)-2,3-dihydro-1H-benzo[d]imidazol-1-yl)piperidine-2,6-dione

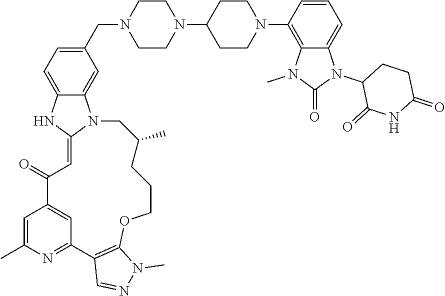 [SMILES: Cc1cc2cc(n1)-c1cnn(C)c1OCCC[C@@H](C)CN1/C(=C/C2=O)Nc2ccc(CN3CCN(C4CCN(c5cccc6c5n(C)c(=O)n6C5CCC(=O)NC5=O)CC4)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (238 mg, 0.45 mmol), 3-(3-methyl-2-oxo-4-(4-oxopiperidin-1-yl)-2,3-dihydro-1H-benzo[d]imidazol-1-yl)piperidine-2,6-di one (45.2 mg, 0.15 mmol) and DCE (8 mL). To the reaction mixture was added N,N-diethylpropylethylamine (0.5 mL, 3.0 mmol) and Ti(OiPr)₄ (0.3 mL, 1.0 mmol). After stirring for 12 h at 70° C., the reaction mixture was cooled down to room temperature and NaBH(OAc)₃ (127 mg, 0.6 mmol) was added. After stirring for 2 h at 70° C., the reaction mixture was concentrated under reduced pressure. Purification by flash column chromatography (0-70% MeOH in DCM) followed by prep-HPLC chromatography (0.1% FA in water:acetonitrile=90:10˜ 50:50 gradient elution) afforded the title compound (38 mg, 45%). ¹H NMR (500 MHz, DMSO) δ 12.67 (s, 1H), 11.04 (s, 1H), 8.36 (s, 1H), 7.86 (s, 1H), 7.50 (s, 1H), 7.46 (s, 1H), 7.43 (d, J=8.0 Hz, 1H), 7.13 (d, J=8.0 Hz, 1H), 6.90 (t, J=8.0 Hz, 1H), 6.82 (d, J=8.0 Hz, 1H), 6.81 (d, J=8.0 Hz, 1H), 4.28 (dd, J=12.5, 4.5 Hz, 1H), 4.32-4.27 (m, 1H), 4.14 (d, J=12.5 Hz, 1H), 3.94-3.90 (m, 1H), 3.87 (t, J=12.5 Hz, 1H), 3.67 (s, 3H), 3.55 (s, 3H), 3.12-3.05 (m, 2H), 2.86-2.70 (m, 3H), 2.65-2.53 (m, 7H), 2.49 (s, 3H), 2.45-2.30 (m, 7H), 2.21-2.10 (m, 1H), 1.95-1.85 (m, 5H), 1.67-1.51 (m, 2H), 1.45-1.32 (m, 1H), 0.75 (d, J=6.5 Hz, 3H); [M+H]⁺=869.8.

### Example 16: (R)-3-(2,6-difluoro-4-(1′-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-[1,4′-bipiperidin]-4-yl)phenyl)piperidine-2,6-dione

### Step 1: (R,E)-5⁶-((1,4-dioxa-8-azaspiro[4.5]decan-8-yl)methyl)-1¹,2⁶,7-trimethyl-5²,5³-dihydro-1¹H,5¹H-11-oxa-4-aza-5(2,1)-benzo[d]imidazola-2(2,4)pyridina-1(4,5)-pyrazolacycloundecaphan-3-one

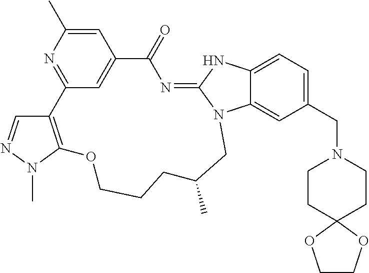 [SMILES: Cc1cc2cc(n1)-c1cnn(C)c1OCCC[C@@H](C)CN1/C(=N/C2=O)Nc2ccc(CN3CCC4(CC3)OCCO4)cc21]

To a 100-mL round-bottomed flask equipped with a magnetic stir bar was added (R,E)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (916 mg, 2.0 mmol), 1,4-dioxa-8-azaspiro[4.5]decane (2.3 g, 16.0 mmol), NaBH(OAc)₃ (2.1 g, 10.0 mmol) and DCM (50 mL) at room temperature. To the stirred reaction mixture was added AcOH (0.69 mL, 12.0 mmol). After stirring for 12 h at room temperature, the reaction mixture was diluted with sat. aq. NaHCO₃(50 mL) and the layers were separated. The aqueous layer was extracted with DCM (3×40 mL). The combined organic layers were washed with brine (100 mL), dried over anhydrous Na₂SO₄, filtered and concentrated under reduced pressure. Purification by flash column chromatography (0-10% MeOH in DCM) afforded the product (1.0 g, 85%). [M+H]⁺=586.5.

### Step 2: (R,E)-1¹,2⁶,7-trimethyl-5⁶-((4-oxopiperidin-1-v,)methyl)-5²,5³-dihydro-1¹H,5¹H-11-oxa-4-aza-5(2,1) -benzo[d]imidazola-2(2,4)-pyridina-1(4,5)-pyrazolacycloundecaphan-3-one

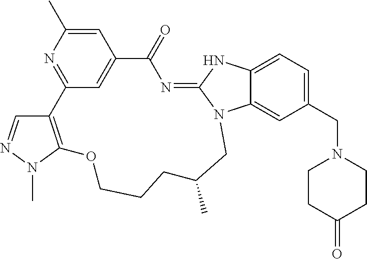 [SMILES: Cc1cc2cc(n1)-c1cnn(C)c1OCCC[C@@H](C)CN1/C(=N/C2=O)Nc2ccc(CN3CCC(=O)CC3)cc21]

To a 100-mL round-bottomed flask equipped with a magnetic stir bar was added (R,E)-5⁶-((1,4-dioxa-8-azaspiro[4.5]decan-8-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (1.0 g, 1.7 mmol) and 6 N HCl (40 mL). After stirring for 3 h at room temperature, the reaction mixture was concentrated under reduced pressure to afford the product (670 mg, 70%), which was used without further purification. [M+H₂O+H]⁺=560.4.

### Step 3: (R)-3-(2,6-difluoro-4-(1′-(((R,E)-1¹,2⁶,7-trimethyl-3-oxo-5²,5³-dihydro-1¹H,5¹H-11-oxa-4-aza-5(2,1) -benzo[d]imidazola-2(2,4)-pyridina-1(4,5)-pyrazolacycloundecaphane-5⁶-yl)methyl)-[1,4′-bipiperidin]-4-yl)phenyl)piperidine-2,6-dione

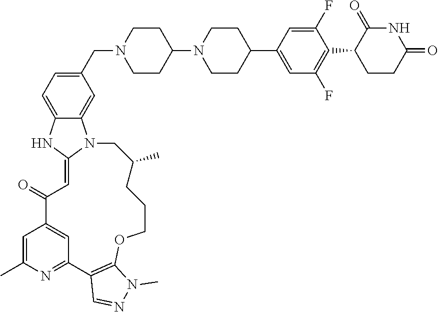 [SMILES: Cc1cc2cc(n1)-c1cnn(C)c1OCCC[C@@H](C)CN1/C(=C/C2=O)Nc2ccc(CN3CCC(N4CCC(c5cc(F)c([C@H]6CCC(=O)NC6=O)c(F)c5)CC4)CC3)cc21]

To a 40-mL microwave vial equipped with a magnetic stir bar was added (R,E)-1¹,2⁶,7-trimethyl-5⁶-((4-oxopiperidin-1-yl)methyl)-5²,5³-dihydro-1¹H,5¹H-11-oxa-4-aza-5(2,1) -benzo[d]imidazola-2(2,4)-pyridina-1(4,5)-pyrazolacycloundecaphan-3-one (167 mg, 0.30 mmol), (R)-3-(2,6-difluoro-4-(piperidin-4-yl)phenyl)piperidine-2,6-dione (the compound was obtained through the similar way in example 14) (30.8 mg, 0.10 mmol) and DCE (8 mL). To the reaction mixture was added N,N-diethylpropylethylamine (0.5 mL, 3.0 mmol) and Ti(OiPr)₄ (0.3 mL, 1.0 mmol). After stirring for 12 h at 70° C., the reaction mixture was cooled down to room temperature and NaBH(OAc)₃ (106 mg, 0.5 mmol) was added. After stirring for 2 h at 70° C., the reaction mixture was concentrated under reduced pressure. Purification by flash column chromatography (0-70% MeOH in DCM) followed by prep-HPLC chromatography (0.1% FA in water; acetonitrile=90:10˜ 50:50 gradient elution) afforded the title compound (25 mg, 30%). ¹H NMR (500 MHz, DMSO) δ 12.74 (s, 1H), 10.98 (s, 1H), 8.44 (s, 1H), 7.93 (s, 1H), 7.63-7.49 (m, 3H), 7.23-7.13 (m, 1H), 7.02 (d, J=10.0 Hz, 2H), 4.44-4.27 (m, 2H), 4.24-4.16 (m, 1H), 4.03-3.89 (m, 2H), 3.74 (s, 3H), 3.09-2.87 (m, 7H), 2.85-2.76 (m, 3H), 2.56 (s, 3H), 2.27-2.05 (m, 5H), 2.03-1.86 (m, 8H), 1.77-1.55 (m, 4H), 0.88-0.77 (m, 2H), 0.82 (d, J=5.0 Hz, 3H); [M+H]⁺=834.5.

### Example 17: (R)-3-(2,6-difluoro-4-(3-(9-(((R,E)-1¹,2⁶,7-trimethyl-3-oxo-5²,5³-dihydro-1¹H,5¹H-11-oxa-4-aza-5(2, 1)-benzo[d]imidazola-2(2,4)-pyridina-1(4,5)-pyrazolacycloundecaphane-5⁶-yl)methyl)-3,9-diazaspiro[5.5]undecan-3-yl)azetidin-1-yl)phenyl)piperdine-2,6-dione

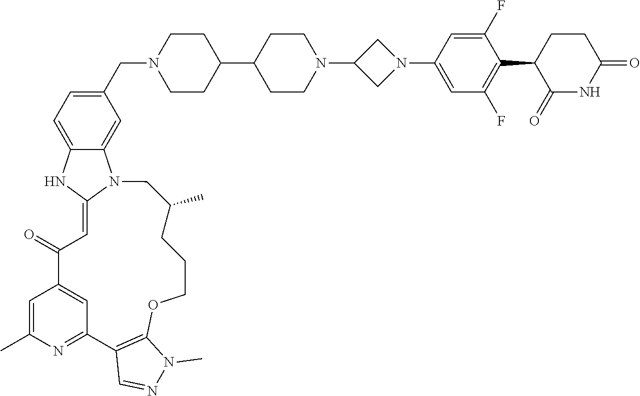 [SMILES: Cc1cc2cc(n1)-c1cnn(C)c1OCCC[C@@H](C)CN1/C(=C/C2=O)Nc2ccc(CN3CCC(C4CCN(C5CN(c6cc(F)c([C@H]7CCC(=O)NC7=O)c(F)c6)C5)CC4)CC3)cc21]

To a 40-mL microwave vial equipped with a magnetic stir bar was added (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (143 mg, 0.24 mmol), (R)-3-(2,6-difluoro-4-(3-oxoazetidin-1-yl)phenyl)piperidine-2,6-dione (the compound was obtained through the similar way of example 5) (58.8 mg, 0.2 mmol) and DCE (8 mL). To the reaction mixture was added NaBH(OAc)z (84.8 mg, 0.4 mmol) and 3 drops of AcOH. After stirring for 4 h at 70° C., the reaction mixture was concentrated under reduced pressure. Purification by flash column chromatography (0-70% MeOH in DCM) followed by prep-HPLC chromatography (0.1% FA in water:acetonitrile=90:10-50:50 gradient elution) afforded the title compound (22 mg, 13%). ¹H NMR (500 MHz, DMSO) δ 12.79 (s, 1H), 10.88 (s, 1H), 8.43 (s, 1H), 8.22 (s, 2H), 7.94 (s, 1H), 7.62-7.54 (m, 2H), 6.11 (d, J=11.5 Hz, 2H), 4.44-4.32 (m, 2H), 4.26-4.18 (m, 1H), 4.06-3.97 (m, 2H), 3.94-3.84 (m, 3H), 3.74 (s, 3H), 3.64-3.53 (m, 3H), 2.98-2.86 (m, 5H), 2.84-2.73 (m, 2H), 2.56 (s, 3H), 2.34-2.17 (m, 5H), 2.11-1.89 (m, 6H), 1.53-1.35 (m, 8H), 0.84 (d, J=6.5 Hz, 3H); [M+H]⁺=875.5.

### Example 18: (R)-3-(2,6-difluoro-4-(3-(4-(1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)piperazin-1-yl)azetidin-1-yl)phenyl)piperidine-2,6-dione

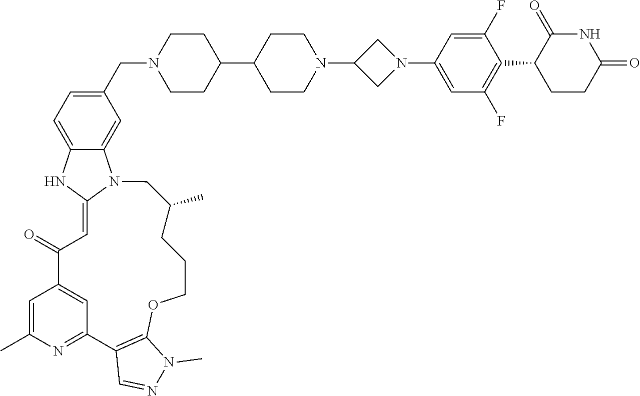 [SMILES: Cc1cc2cc(n1)-c1cnn(C)c1OCCC[C@@H](C)CN1/C(=C/C2=O)Nc2ccc(CN3CCC(C4CCN(C5CN(c6cc(F)c([C@H]7CCC(=O)NC7=O)c(F)c6)C5)CC4)CC3)cc21]

To a 40-mL microwave vial equipped with a magnetic stir bar was added (R,E)-1¹,2⁶,7-trimethyl-5⁶-((4-(piperazin-1-yl)piperidin-1-yl)methyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (146 mg, 0.24 mmol), (R)-3-(2,6-difluoro-4-(3-oxoazetidin-1-yl)phenyl)piperidine-2,6-dione (58.8 mg, 0.2 mmol) and DCE (8 mL). To the reaction mixture was added NaBH(OAc)₃ (84.8 mg, 0.4 mmol) and 3 drops of AcOH. After stirring for 4 h at 70° C., the reaction mixture was concentrated under reduced pressure. Purification by flash column chromatography (0-70% MeOH in DCM) followed by prep-HPLC chromatography (0.1% FA in water:acetonitrile=90:10-50:50 gradient elution) afforded the title compound (34 mg, 19%). ¹H NMR (500 MHz, DMSO) δ 12.73 (s, 1H), 10.87 (s, 1H), 8.43 (s, 1H), 7.92 (s, 1H), 7.57 (s, 1H), 7.51-7.47 (m, 2H), 7.22-7.10 (m, 1H), 6.11 (d, J=11.0 Hz, 2H), 4.43-4.28 (m, 1H), 4.22-4.16 (m, 1H), 4.05-3.98 (m, 2H), 3.95-3.84 (m, 3H), 3.73 (s, 3H), 3.65-3.55 (m, 3H), 3.01-2.86 (m, 4H), 2.83-2.71 (m, 2H), 2.56 (s, 3H), 2.50-2.43 (m, 5H), 2.34-2.16 (m, 5H), 2.10-1.85 (m, 7H), 1.31-1.20 (m, 5H), 0.81 (d, J=6.5 Hz, 3H); [M+H]⁺=890.6.

### Example 20: (R)-3-(2,6-difluoro-4-((R)-3-(7-(((R,E)-1¹,2⁶,7-trimethyl-3-oxo-5²,5³-dihydro-1¹H,5¹H-li-oxa-4-aza -5(2,1)-benzo[d]imidazola-2(2,4)-pyridina-1(4,5)-pyrazolacycloundecaphane-5⁶-yl)methyl)-2,7-diazaspiro[3.5]nonane-2-carbonyl)pyrrolidin-1-yl)phenyl)piperidine-2,6-dione

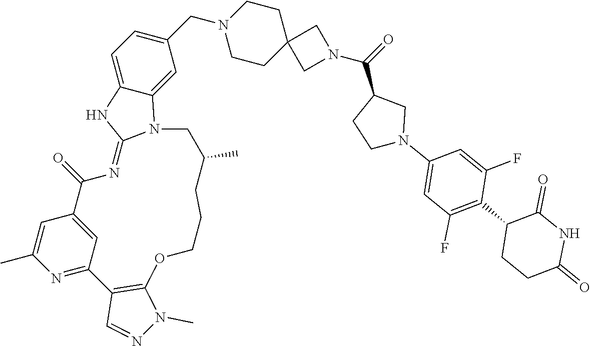 [SMILES: Cc1cc2cc(n1)-c1cnn(C)c1OCCC[C@@H](C)CN1/C(=N/C2=O)Nc2ccc(CN3CCC4(CC3)CN(C(=O)[C@@H]3CCN(c5cc(F)c([C@H]6CCC(=O)NC6=O)c(F)c5)C3)C4)cc21]

The titled compound was prepared in a manner similar to that in Example 5. ¹H NMR (500 MHz, DMSO) δ 12.74 (s, 1H), 10.86 (s, 1H), 8.43 (s, 1H), 7.92 (s, 1H), 7.54-7.51 (m, 3H), 7.20 (s, 1H), 6.22 (d, J=12.3 Hz, 2H), 4.36 (d, J=4.6 Hz, 1H), 4.22 (d, J=11.9 Hz, 1H), 4.03-3.98 (m, 2H), 3.92 (s, 3H), 3.73 (s, 3H), 3.57 (s, 3H), 3.44-3.40 (m, 2H), 3.31-3.27 (m, 2H), 3.25-3.20 (m, 2H), 3.17-3.12 (m, 1H), 2.86-2.68 (m, 2H), 2.55 (d, J=8.3 Hz, 3H), 2.50-2.46 (m, 3H), 2.22 (s, 2H), 2.17-1.97 (m, 5H), 1.96-1.85 (m, 2H), 1.74 (s, 3H), 1.46 (s, 1H), 0.82 (d, J=5.9 Hz, 3H). [M+H]⁺=889.6.

### Example 21: 3-(1-oxo-5-(4-((9-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecan-3-yl)methyl)piperidin-1-yl)isoindolin-2-yl)piperidine-2,6-dione

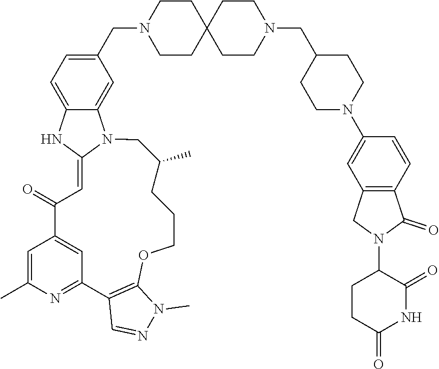 [SMILES: Cc1cc2cc(n1)-c1cnn(C)c1OCCC[C@@H](C)CN1/C(=C/C2=O)Nc2ccc(CN3CCC4(CC3)CCN(CC3CCN(c5ccc6c(c5)CN(C5CCC(=O)NC5=O)C6=O)CC3)CC4)cc21]

The titled compound was prepared in a manner similar to that in Example 14. ¹H NMR (500 MHz, DMSO) δ 12.69 (s, 1H), 10.93 (s, 1H), 8.43 (s, 1H), 7.92 (s, 1H), 7.57 (s, 1H), 7.48 (t, J=8.9 Hz, 3H), 7.16 (d, J=8.0 Hz, 1H), 7.02 (d, J=8.5 Hz, 2H), 5.03 (dd, J=13.5, 5.1 Hz, 1H), 4.40-4.27 (m, 2H), 4.23-4.13 (m, 2H), 4.00 (s, 1H), 3.97-3.90 (m, 1H), 3.85 (d, J=11.0 Hz, 2H), 3.73 (s, 3H), 3.55 (s, 2H), 2.96-2.85 (m, 1H), 2.80 (t, J=11.9 Hz, 3H), 2.65-2.54 (m, 5H), 2.42-2.26 (m, 9H), 2.25-2.18 (m, 1H), 2.16-2.09 (m, 2H), 2.03-1.87 (m, 3H), 1.75 (t, J=11.6 Hz, 3H), 1.44-1.41 (m, 8H), 1.15 (d, J=11.6 Hz, 2H), 0.81 (d, J=6.4 Hz, 3H); [M+H]⁺=936.8.

### Example 22: 3-(4-(methyl(2-(9-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecan-3-yl)ethyl)amino)-1-oxoisoindolin-2-yl)piperidine-2,6-dione

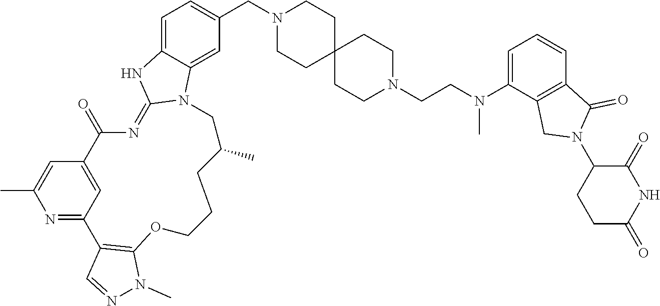 [SMILES: Cc1cc2cc(n1)-c1cnn(C)c1OCCC[C@@H](C)CN1/C(=N/C2=O)Nc2ccc(CN3CCC4(CCN(CCN(C)c5cccc6c5CN(C5CCC(=O)NC5=O)C6=O)CC4)CC3)cc21]

The titled compound was prepared in a manner similar to that in Example 14.

¹H NMR (500 MHz, DMSO) δ 12.69 (s, 1H), 10.98 (s, 1H), 8.42 (s, 1H), 7.92 (s, 1H), 7.56 (s, 1H), 7.46 (d, J=8.5 Hz, 2H), 7.35 (t, J=7.7 Hz, 1H), 7.14 (t, J=7.2 Hz, 2H), 6.98 (d, J=8.0 Hz, 1H), 5.11 (dd, J=13.3, 5.3 Hz, 1H), 4.50 (d, J=17.0 Hz, 1H), 4.40-4.30 (m, 2H), 4.19 (d, J=11.0 Hz, 1H), 4.00 (s, 1H), 3.91 (d, J=12.9 Hz, 1H), 3.73 (s, 3H), 3.55 (s, 2H), 3.49-3.36 (m, 4H), 2.92 (s, 4H), 2.78 (s, 1H), 2.65-2.60 (m, 1H), 2.59-2.54 (m, 4H), 2.44-2.39 (m, 2H), 2.37-2.29 (m, 7H), 2.24-2.16 (m, 1H), 2.03-1.87 (m, 3H), 1.52-1.43 (m, 1H), 1.36-1.32 (m, 7H), 0.80 (d, J=6.5 Hz, 3H); [M+H]⁺=896.8.

### Example 23: (R)-3-(2,6-difluoro-4-(2-(7-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,7-diazaspiro[3.5]nonan-2-yl)ethyl)phenyl)piperdine-2,6-dione

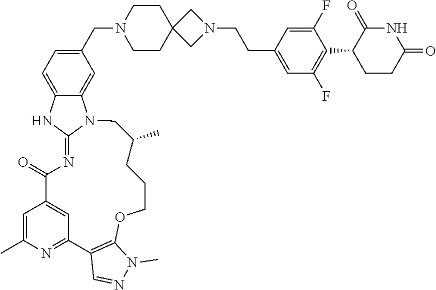 [SMILES: Cc1cc2cc(n1)-c1cnn(C)c1OCCC[C@@H](C)CN1/C(=N/C2=O)Nc2ccc(CN3CCC4(CC3)CN(CCc3cc(F)c([C@H]5CCC(=O)NC5=O)c(F)c3)C4)cc21]

The titled compound was prepared in a manner similar to that in Example 14.

¹H NMR (500 MHz, DMSO) δ 12.71 (s, 1H), 10.96 (s, 1H), 8.43 (s, 1H), 7.91 (d, J=7.2 Hz, 1H), 7.57 (s, 1H), 7.53-7.39 (m, 2H), 7.15 (d, J=13.6 Hz, 1H), 6.98 (d, J=10.1 Hz, 2H), 4.38-4.34 (m, 1H), 4.19 (d, J=12.6 Hz, 2H), 4.03-3.92 (m, 2H), 3.73 (s, 3H), 3.52 (s, 2H), 2.93 (s, 4H), 2.85-2.72 (m, 2H), 2.70-2.59 (m, 2H), 2.59-2.54 (m, 6H), 2.39-2.18 (m, 5H), 2.16-1.82 (m, 4H), 1.68-1.64 (m, 4H), 1.45 (s, 1H), 0.80 (d, J=6.4 Hz, 3H). [M+H]⁺=820.8.

### Example 24: 2-(2,6-dioxopiperidin-3-yl)-5-(4-((9-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecan-3-yl)methyl)piperidin-1-yl)isoindoline-1,3-dione

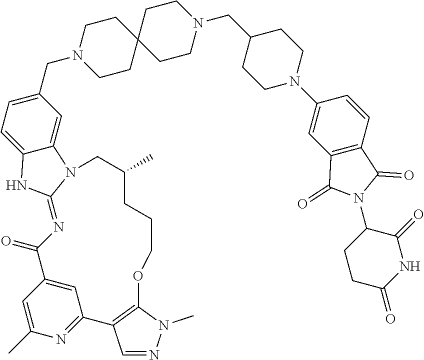 [SMILES: Cc1cc2cc(n1)-c1cnn(C)c1OCCC[C@@H](C)CN1/C(=N/C2=O)Nc2ccc(CN3CCC4(CC3)CCN(CC3CCN(c5ccc6c(c5)C(=O)N(C5CCC(=O)NC5=O)C6=O)CC3)CC4)cc21]

The titled compound was prepared in a manner similar to that in Example 14.

¹H NMR (500 MHz, DMSO) δ 12.70 (s, 1H), 11.07 (s, 1H), 8.43 (s, 1H), 7.92 (s, 1H), 7.64 (d, J=8.3 Hz, 1H), 7.56 (s, 1H), 7.47 (d, J=11.8 Hz, 2H), 7.29 (s, 1H), 7.21 (d, J=8.5 Hz, 1H), 7.16 (d, J=7.5 Hz, 1H), 5.06 (dd, J=12.7, 5.4 Hz, 1H), 4.36 (dd, J=14.9, 6.3 Hz, 1H), 4.20 (d, J=13.4 Hz, 1H), 4.07-3.97 (m, 3H), 3.97-3.86 (m, 1H), 3.73 (s, 3H), 3.56 (s, 2H), 2.99-2.83 (m, 3H), 2.82-2.72 (m, 1H), 2.66-2.53 (m, 6H), 2.39-2.27 (m, 7H), 2.25-2.08 (m, 3H), 2.06-1.87 (m, 3H), 1.84-1.71 (m, 3H), 1.51-1.35 (m, 9H), 1.18-1.05 (m, 2H), 0.81 (d, J=6.3 Hz, 3H); [M+H]⁺=950.8.

### Example 25: (R)-3-(2,6-difluoro-4-(3-(7-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,7-diazaspiro[3.5]nonane-2-carbonyl)azetidin-1-yl)phenyl)piperidine-2,6-dione

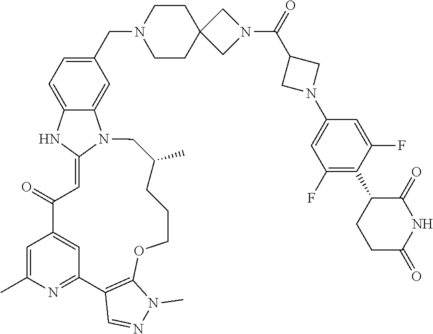 [SMILES: Cc1cc2cc(n1)-c1cnn(C)c1OCCC[C@@H](C)CN1/C(=C/C2=O)Nc2ccc(CN3CCC4(CC3)CN(C(=O)C3CN(c5cc(F)c([C@H]6CCC(=O)NC6=O)c(F)c5)C3)C4)cc21]

The titled compound was prepared in a manner similar to that in Example 5.

¹H NMR (500 MHz, DMSO) δ 12.72 (s, 1H), 10.85 (s, 1H), 8.43 (s, 1H), 7.92 (s, 1H), 7.57 (s, 1H), 7.50 (s, 2H), 7.19 (s, 1H), 6.14 (d, J=11.1 Hz, 2H), 4.36 (d, J=4.2 Hz, 1H), 4.21 (d, J=12.4 Hz, 1H), 4.09-3.88 (m, 5H), 3.83 (t, J=6.2 Hz, 2H), 3.77 (s, 2H), 3.73 (s, 3H), 3.64-3.48 (m, 4H), 3.30-3.18 (m, 3H), 2.80-2.74 (m, 2H), 2.56 (s, 3H), 2.30-2.18 (m, 3H), 2.14-1.86 (m, 5H), 1.72 (s, 4H), 1.52-1.43 (m, 1H), 0.82 (d, J=6.3 Hz, 3H). [M+H]⁺=875.6.

### Example 26: (R)-3-(4-((R)-3,3-dimethyl-4-(7-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-(4,5)-pyrazolacycloundecaphane-5⁶-yl)methyl)-2,7-diazaspiro[3.5]nonane-2-carbonyl)pyrrolidin-1-yl)-2,6-difluorophenyl)piperidine-2,6-dione

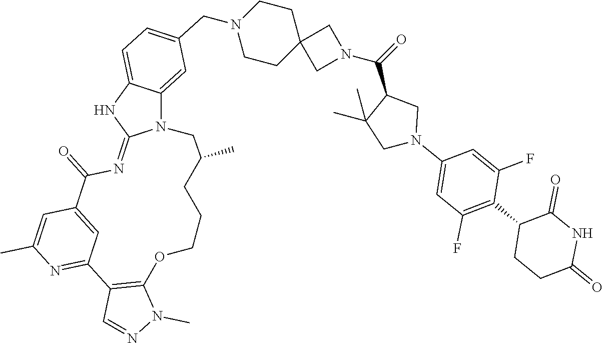 [SMILES: Cc1cc2cc(n1)-c1cnn(C)c1OCCC[C@@H](C)CN1/C(=N/C2=O)Nc2ccc(CN3CCC4(CC3)CN(C(=O)[C@H]3CN(c5cc(F)c([C@H]6CCC(=O)NC6=O)c(F)c5)CC3(C)C)C4)cc21]

The titled compound was prepared in a manner similar to that in Example 5.

¹H NMR (500 MHz, DMSO) δ 12.70 (s, 1H), 10.83 (s, 1H), 8.43 (s, 1H), 7.92 (s, 1H), 7.57 (s, 1H), 7.54-7.39 (m, 2H), 7.17 (d, J=8.0 Hz, 1H), 6.14 (d, J=12.5 Hz, 2H), 4.36 (s, 1H), 4.20 (d, J=11.9 Hz, H), 4.00 (d, J=7.1 Hz, 2H), 3.96-3.88 (m, 2H), 3.83 (d, J=8.2 Hz, 1H), 3.73 (s, 3H), 3.54 (d, J=15.6 Hz, 4H), 3.46-3.41 (m, 1H), 3.18-3.01 (m, 3H), 2.83-2.75 (m, 3H), 2.56 (s, 3H), 2.40-2.18 (m, 5H), 2.14-1.90 (m, 4H), 1.71 (s, 4H), 1.46 (s, 1H), 1.23 (s, 1H), 1.14 (s, 3H), 1.00 (s, 3H), 0.81 (d, J=6.1 Hz, 3H). [M+H]⁺=917.8.

### Example 27: 3-(3-methyl-2-oxo-4-(2-(9-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecan-3-yl)ethyl)-2,3-dihydro-1H-benzo[d]imidazol-1-yl)piperidine-2,6-dione

### Step 1: 7-bromo-1-methyl-1,3-dihydro-2H-benzo[d]imidazol-2-one

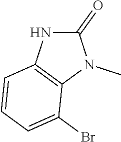

To a solution of 6-bromo-N¹-methylbenzene-1,2-diamine (4 g, 19.9 mmol) in CH₃CN (50 mL) was added CDI (6.4 g, 39.8 mmol). The resulting solution was stirred for 6 h at 90° C. under nitrogen atmosphere. The solid was collected by filtration. This was resulted in 7-bromo-1-methyl-1,3-dihydro-2H-benzo[d]imidazol-2-one (4.1 g, 90.7%). [M+H]⁺=227.0.

### Step 2: 3-(4-bromo-3-methyl-2-oxo-2,3-dihydro-1H-benzo[d]imidazol-1-yl)-1-(4-methoxybenzyl)piperidine -2,6-dione

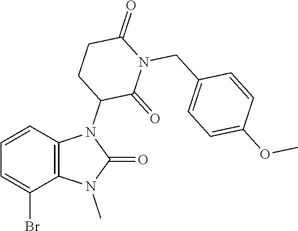 [SMILES: COc1ccc(CN2C(=O)CCC(n3c(=O)n(C)c4c(Br)cccc43)C2=O)cc1]

To a solution of 7-bromo-1-methyl-1,3-dihydro-2H-benzo[d]imidazol-2-one (600 mg, 2.6 mmol) in THF (10 mL) was added t-BuOK (1M in THF, 3.2 mL, 3.1 mmol) dropwise in 10 min at 0° C. the reaction solution was stirred for 30 min at this temperature, then to this was added 1-(4-methoxybenzyl)-2,6-dioxopiperidin-3-yl trifluoromethanesulfonate (1.1 g, 2.9 mmol) in THF (5 mL) dropwise in 10 min. The resulting solution was stirred for 2 h at 0-10 degrees C. The reaction was quenched by the addition of sat. aq. NH₄Cl solution, extracted with EtOAc (10 mL×3), combined the organic layer, and washed with brine, dried over anhydrous Na₂SO₄, after filtration, the filtrate was concentrated under reduced pressure. The residue was purified by a silica gel column, eluted with PE/EtOAc to afford product (910 mg, 75.2%). [M+H]⁺=458.1.

### Step 3: (E)-3-(4-bromo-3-methyl-2-oxo-2,3-dihydro-1H-benzo[d]imidazol-1-yl)piperidine-2,6-dione

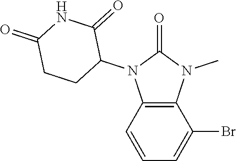 [SMILES: Cn1c(=O)n(C2CCC(=O)NC2=O)c2cccc(Br)c21]

3-(4-bromo-3-methyl-2-oxo-2,3-dihydro-1H-benzo[d]imidazol-1-yl)-1-(4-methoxybenzyl)pi peridine-2,6-dione (800 mg, 1.75 mmol) was dissolved in MeSO₂H/toluene (2 mL/6 mL). The resulting mixture was stirred for 3 h at 100° C. Solvent was removed and the residue was poured into ice/water. The solid was collected by filtration. 3-(4-bromo-3-methyl-2-oxo-2,3-dihydro-1H-benzo[d]imidazol-1-yl)piperidine-2,6-dione was obtained (510 mg, 86.4%). [M+H]⁺=338.1.

### Step 4: (E)-3-(4-(2-ethoxyvinyl)-3-methyl-2-oxo-2,3-dihydro-1H-benzo[d]imidazol-1-yl)piperidine-2,6-dione

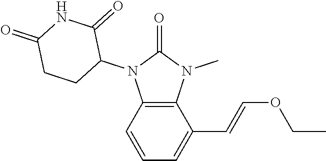 [SMILES: CCO/C=C/c1cccc2c1n(C)c(=O)n2C1CCC(=O)NC1=O]

To a stirred solution of 3-(4-bromo-3-methyl-2-oxo-2,3-dihydro-1H-benzo[d]imidazol-1-yl)piperidine-2,6-dione (250 mg, 0.74 mmol) and (E)-2-(2-ethoxyvinyl)-4,4,5,5-tetramethyl-1,3,2-dioxaborolane (176 mg, 0.89 mmol) in DMF/H₂O (8 mL/2 mL) were added Pd(dtbpf)Cl₂ (48 mg, 0.074 mmol) and CsF (225 mg, 1.48 mmol). The resulting mixture was stirred for 2 h at 80° C. under nitrogen atmosphere. The reaction solution was diluted with water, extracted with EtOAc (10 mL×3). The organic layer was washed with water and brine, dried over anhydrous Na₂SO₄ which was evaporated to dryness. The residue was purified by a silica gel column, eluted with PE/EtOAc=1:1 to afford the product. (180 mg, 73.8%), m/z [M+H]⁺=330.2.

### Step 5: 2-(1-(2,6-dioxopiperidin-3-yl-3-methyl-2-oxo-2,3-dihydro-1H-benzo[d]imidazol-4-yl)acetaldehyde

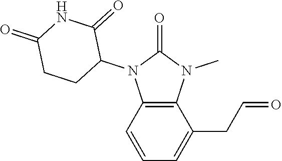 [SMILES: Cn1c(=O)n(C2CCC(=O)NC2=O)c2cccc(CC=O)c21]

(E)-3-(4-(2-ethoxyvinyl)-3-methyl-2-oxo-2,3-dihydro-1H-benzo[d]imidazol-1-yl)piperidine -2,6-dione (180 mg, 0.55 mmol) was dissolved in HCOOH (2 mL). The resulting solution was stirred for 2 h at room temperature. The reaction solution was evaporated to dryness to afford product (125 mg, 75.3%) which was used directly in the next step, m/z [M+H]⁺=302.1.

### Step 6: 3-(3-methyl-2-oxo-4-(2-(9-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁴-yl)methyl)-3,9-diazaspiro[5.5]undecan-3-yl)ethyl)-2,3-dihydro-1H-benzo[d]imidazol-1-yl)piperidine-2,6-dione

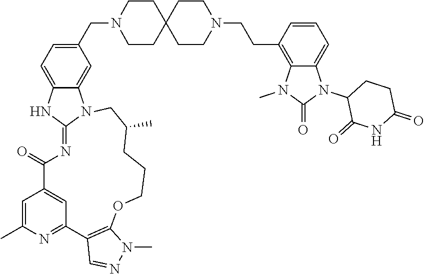 [SMILES: Cc1cc2cc(n1)-c1cnn(C)c1OCCC[C@@H](C)CN1/C(=N/C2=O)Nc2ccc(CN3CCC4(CCN(CCc5cccc6c5n(C)c(=O)n6C5CCC(=O)NC5=O)CC4)CC3)cc21]

To a 40-mL microwave vial equipped with a magnetic stir bar was added (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (59.7 mg, 0.1 mmol), 2-(1-(2,6-dioxopiperidin-3-yl)-3-methyl-2-oxo-2,3-dihydro-1H-benzo[d]imidazol-4-yl)acetaldehyde (60.2 mg, 0.2 mmol), NaOAc (32.8 mg, 0.4 mmol), DMSO (2 mL) and DCE (8 mL). To the reaction mixture was added NaBH(OAc)₃ (63.6 mg, 0.3 mmol). After stirring for 4 h at 70° C., the reaction mixture was diluted with sat. aq. NaHCO₃(20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by prep-TLC followed by prep-HPLC chromatography (0.1% FA in water:acetonitrile=90:10-50:50 gradient elution) afforded the title compound (25 mg, 28%). ¹H NMR (500 MHz, DMSO) δ 12.64 (s, 1H), 11.02 (s, 1H), 8.36 (s, 1H), 7.86 (s, 1H), 7.50 (s, 1H), 7.44 (s, 1H), 7.42 (d, J=8.5 Hz, 1H), 7.11 (d, J=8.0 Hz, 1H), 6.94-6.89 (m, 2H), 6.82 (d, J=8.5 Hz, 1H), 5.29 (dd, J=12.5, 5.0 Hz, 1H), 4.34-4.26 (m, 1H), 4.14 (d, J=12.5 Hz, 1H), 3.97-3.90 (m, 1H), 3.88-3.78 (m, 2H), 3.67 (s, 3H), 3.61-3.55 (m, 4H), 3.51 (s, 3H), 3.20-3.00 (m, 7H), 2.88-2.75 (m, 2H), 2.74-2.61 (m, 5H), 2.60-2.52 (m, 6H), 2.49 (s, 3H), 2.35-2.30 (m, 2H), 2.02-1.83 (m, 4H), 0.74 (d, J=6.0 Hz, 3H); [M+H]⁺=882.8.

### Example 28: 3-(3-methyl-2-oxo-4-(2-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)ethyl)-2,3-dihydro-1H-benzo[d]imidazol-1-yl)piperidine-2,6-dione

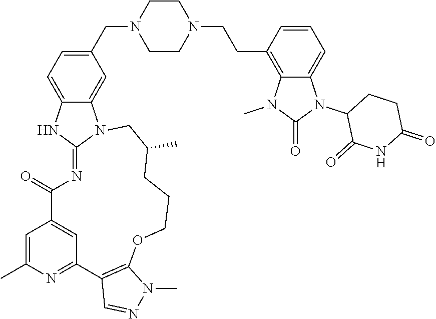 [SMILES: Cc1cc2cc(n1)-c1cnn(C)c1OCCC[C@@H](C)CN1/C(=N/C2=O)Nc2ccc(CN3CCN(CCc4cccc5c4n(C)c(=O)n5C4CCC(=O)NC4=O)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (52.9 mg, 0.1 mmol), 2-(1-(2,6-dioxopiperidin-3-yl)-3-methyl-2-oxo-2,3-dihydro-1H-benzo[d]imidazol-4-yl)acetaldehyde (60.2 mg, 0.2 mmol), NaOAc (32.8 mg, 0.4 mmol), DMSO (2 mL) and DCE (8 mL). To the reaction mixture was added NaBH(OAc)₃ (63.6 mg, 0.3 mmol). After stirring for 4 h at 70° C., the reaction mixture was diluted with sat. aq. NaHCO₃ (20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by prep-TLC followed by prep-HPLC chromatography (0.1% FA in water:acetonitrile=90:10-50:50 gradient elution) afforded the title compound (34 mg, 42%). ¹H NMR (500 MHz, DMSO) δ 12.84 (s, 1H), 11.10 (s, 1H), 8.50 (s, 1H), 8.01 (s, 1H), 7.66 (s, 1H), 7.63 (s, 1H), 7.57 (d, J=8.0 Hz, 1H), 7.28 (d, J=8.5 Hz, 1H), 7.04-7.02 (m, 2H), 6.93 (d, J=7.0 Hz, 1H), 5.38 (dd, J=12.5, 5.0 Hz, 1H), 4.41-4.36 (m, 1H), 4.24 (d, J=11.5 Hz, 1H), 4.07-4.03 (m, 2H), 3.93-3.88 (m, 2H), 3.75 (s, 3H), 3.59 (s, 3H), 3.45-3.35 (m, 9H), 2.93-2.85 (m, 2H), 2.84-2.77 (m, 1H), 2.76-2.67 (m, 2H), 2.66-2.63 (m, 2H), 2.61 (s, 3H), 2.28-2.19 (m, 1H), 2.05-1.87 (m, 3H), 1.51-1.44 (m, 1H), 0.84 (d, J=6.5 Hz, 3H); [M+H]⁺=814.8.

### Example 29: (R)-3-(2,6-difluoro-4-(2-(9-(((R,E)-1¹,2⁶,7-trimethyl-3-oxo-5², 5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,9-diazaspiro[5.5]undecan-3-yl)ethyl)phenyl)piperidine-2,6-dione

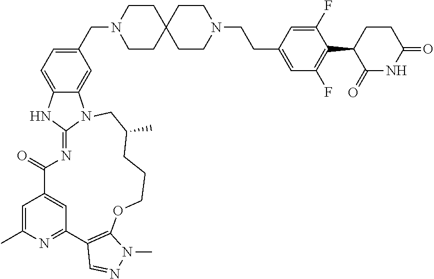 [SMILES: Cc1cc2cc(n1)-c1cnn(C)c1OCCC[C@@H](C)CN1/C(=N/C2=O)Nc2ccc(CN3CCC4(CCN(CCc5cc(F)c([C@H]6CCC(=O)NC6=O)c(F)c5)CC4)CC3)cc21]

To a 40-mL microwave vial equipped with a magnetic stir bar was added (R,E)-5⁶-((3,9-diazaspiro[5.5]undecan-3-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (59.7 mg, 0.10 mmol), (R)-2-(4-(2,6-dioxopiperidin-3-yl)-3,5-difluorophenyl)acetaldehyde (58.7 mg, 0.22 mmol), DMSO (2 mL) and DCE (8 mL). To the reaction mixture was added NaBH(OAc)₃ (63.6 mg, 0.3 mmol) and 6 drops of AcOH. After stirring for 4 h at 70° C., the reaction mixture was diluted with sat. aq. NaHCO₃(20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by prep-TLC followed by prep-HPLC chromatography (0.1% FA in water:acetonitrile=90:10-50:50 gradient elution) afforded the title compound (28 mg, 33%). ¹H NMR (500 MHz, DMSO) δ 12.70 (s, 1H), 10.94 (s, 1H), 8.43 (s, 1H), 7.92 (s, 1H), 7.56 (s, 1H), 7.49 (s, 1H), 7.47 (d, J=8.0 Hz, 1H), 7.16 (d, J=8.0 Hz, 1H), 7.00 (d, J=10.0 Hz, 2H), 4.40-4.31 (m, 1H), 4.23-4.15 (m, 2H), 4.03-3.95 (m, 1H), 3.94-3.89 (m, 1H), 3.73 (s, 3H), 3.60-3.55 (m, 3H), 2.85-2.71 (m, 4H), 2.56 (s, 3H), 2.56-2.53 (m, 3H), 2.45-2.32 (m, 8H), 2.25-2.17 (m, 1H), 2.16-2.06 (m, 1H), 2.00-1.87 (m, 3H), 1.49-1.36 (m, 8H), 0.81 (d, J=6.5 Hz, 3H); [M+H]⁺=848.7.

### Example 30: 3-(3-methyl-2-oxo-4-(2-(7-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,7-diazaspiro[3,5]nonan-2-yl)ethyl)-2,3-dihydro-1H-benzo[d]imidazol-1-yl)piperidine-2,6-dione

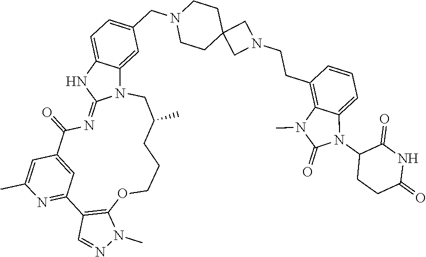 [SMILES: Cc1cc2cc(n1)-c1cnn(C)c1OCCC[C@@H](C)CN1/C(=N/C2=O)Nc2ccc(CN3CCC4(CC3)CN(CCc3cccc5c3n(C)c(=O)n5C3CCC(=O)NC3=O)C4)cc21]

The titled compound was prepared in a manner similar to that in Example 27.

¹H NMR (500 MHz, DMSO) δ 12.70 (s, 1H), 11.08 (s, 1H), 8.43 (s, 1H), 7.92 (s, 1H), 7.57 (s, 1H), 7.53-7.36 (m, 2H), 7.15 (d, J=8.1 Hz, 1H), 6.98-6.90 (m, 2H), 6.87 (d, J=6.7 Hz, 1H), 5.35 (dd, J=12.6, 5.3 Hz, 1H), 4.36 (d, J=4.5 Hz, 1H), 4.20 (d, J=11.6 Hz, 1H), 4.00-3.90 (m, 3H), 3.73 (s, 3H), 3.56 (s, 3H), 3.52 (s, 2H), 2.96 (s, 3H), 2.90-2.85 (m, 3H), 2.79 (s, 1H), 2.69 (d, J=8.0 Hz, 3H), 2.62 (d, J=18.0 Hz, 2H), 2.56 (s, 3H), 2.28 (s, 4H), 2.06-1.84 (m, 3H), 1.66 (s, 4H), 1.46 (s, 1H), 0.81 (d, J=6.4 Hz, 3H). [M+H]⁺=854.6.

### Example 76: (R)-3-(2,6-difluoro-4-(3-(4-(((R,E)-1¹,2⁶,7-trimethyl-3-oxo-5²,5-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carbonyl)azetidin-1-yl)phenyl)piperidine-2,6-dione

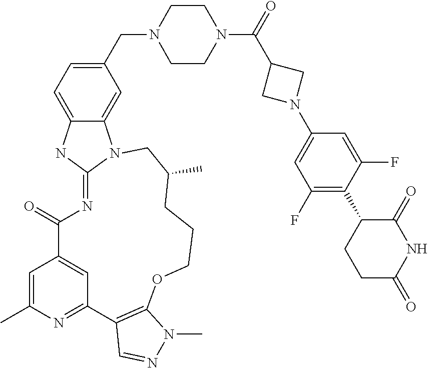 [SMILES: Cc1cc2cc(n1)-c1cnn(C)c1OCCC[C@@H](C)CN1/C(=N/C2=O)Nc2ccc(CN3CCN(C(=O)C4CN(c5cc(F)c([C@H]6CCC(=O)NC6=O)c(F)c5)C4)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (52.9 mg, 0.10 mmol), (R)-1-(4-(2,6-dioxopiperidin-3-yl)-3,5-difluorophenyl)azetidine-3-carboxylic acid (32.4 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (52 mg, 62%). ¹H NMR (500 MHz, DMSO) δ 12.65 (s, 1H), 10.79 (s, 1H), 8.36 (s, 1H), 7.86 (s, 1H), 7.50 (s, 1H), 7.45 (s, 1H), 7.43 (d, J=8.0 Hz, 1H), 7.12 (d, J=8.0 Hz, 1H), 6.08 (d, J=11.0 Hz, 2H), 4.32-4.27 (m, 1H), 4.13 (d, J=11.5 Hz, 1H), 3.98-3.93 (m, 4H), 3.89-3.78 (m, 3H), 3.78-3.72 (m, 1H), 3.66 (s, 3H), 3.54 (s, 2H), 3.43 (s, 2H), 2.74-2.67 (m, 2H), 2.49 (s, 3H), 2.44-2.41 (m, 3H), 2.36-2.25 (m, 4H), 2.20-2.10 (m, 1H), 2.06-1.80 (m, 4H), 1.45-1.35 (m, 1H), 0.75 (d, J=6.0 Hz, 3H); [M+H]⁺=835.5.

### Example 77: (R)-3-(4-((R)-3,3-dimethyl-4-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)methyl)piperazine-1-carbonyl)pyrrolidin-1-yl)-2,6-difluorophenyl)piperidine-2,6-dione

### Step 1: methyl (R)-44-dimethylpyrrolidine-3-carboxylate

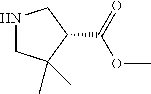

To a stirred solution of (R)-4,4-dimethylpyrrolidine-3-carboxylic acid (2 g, 13.96 mmol) in MeOH (30 mL) was added SOCl₂ (1.66 g, 13.96 mmol) dropwise at 0° C. under nitrogen atmosphere. The resulting mixture was stirred for 2 hrs at 60° C. temperature. The resulting mixture was concentrated under reduced pressure to afford the product (2.1 g, 95.8%) which was used for next step without further purification. [M+H]⁺=158.1

### Step 2: methyl (R)-1-(4-(2,6-bis(benzyloxy)pyridin-3-yl)-3,5-difluorophenyl)-4,4-dimethylpyrrolidine-3-carboxylate

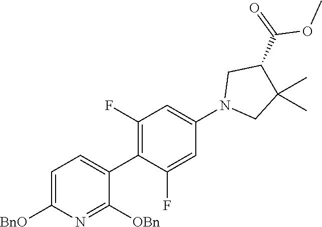 [SMILES: COC(=O)[C@H]1CN(c2cc(F)c(-c3ccc(OCc4ccccc4)nc3OCc3ccccc3)c(F)c2)CC1(C)C]

To a stirred solution of 2,6-bis(benzyloxy)-3-(4-bromo-2,6-difluorophenyl)pyridine (9.25 g, 20.25 mmol) and methyl (R)-4,4-dimethylpyrrolidine-3-carboxylate (2.1 g, 13.35 mmol) in dioxane (50 mL) were added Cs₂CO₃ (10.95 g, 33.37 mmol), Xantphos (1.54 g, 2.67 mmol) and Pd₂(dba)₃ (1.22 g, 1.35 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (500 mL), washed with water (3×200 mL) and brine (200 mL). The organic layer was dried over anhydrous Na₂SO₄, filtered and concentrated under reduced pressure. The residue was purified by silica gel column chromatography, eluted with PE/EtOAc (5:1) to afford the product (4.5 g, 60.8%), [M+H]⁺=559.6

### Step 3: (R)-1-(4-(4-(2,6-bis(benzyloxy)pyridin-3-yl)-3,5-difluorophenyl)-4,4-dimethylpyrrolidine-3-carboxylic acid

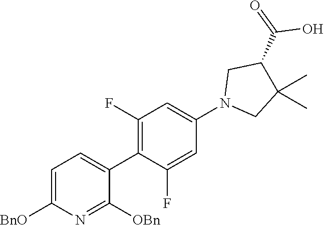 [SMILES: CC1(C)CN(c2cc(F)c(-c3ccc(OCc4ccccc4)nc3OCc3ccccc3)c(F)c2)C[C@@H]1C(=O)O]

To a solution of methyl (R)-1-(4-(2,6-bis(benzyloxy)pyridin-3-yl)-3,5-difluorophenyl)-4,4-dimethylpyrrolidine-3-carboxylate (4.5 g, 8.05 mmol) in THF (40 mL) and H₂O (10 mL) was added lithium hydroxide hydrate (337.9 mg, 8.05 mmol) at 25° C. The resulting mixture was stirred at 25° C. for 5 h. The reaction was quenched with HCl (I N) at 0° C. until pH=6 and the resulting mixture was extracted with EA (2×40 mL). The combined organic layers were washed with brine, dried over anhydrous Na₂SO₄, filtered and concentrated under vacuum to afford the crude product (4.05 g, 92.46%), which was used for next step without further purification. [M+H]⁺=545.6

### Step 4: (R)-1-(4-((R)-2,6-dioxopiperidin-3-yl)-3,5-difluorophenyl)-4,4-dimethylpyrrolidine-3-carboxylic acid

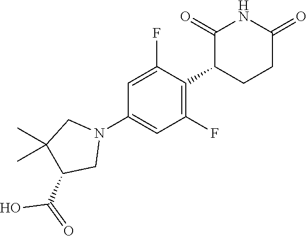 [SMILES: CC1(C)CN(c2cc(F)c([C@H]3CCC(=O)NC3=O)c(F)c2)C[C@@H]1C(=O)O]

(R)-1-(4-(2,6-bis(benzyloxy)pyridin-3-yl)-3,5-difluorophenyl)-4,4-dimethylpyrrolidine-3-carboxylic acid (4.5 g, 8.25 mmol) was dissolved in DCM (30 mL) and iPr-OH (30 mL). Pd/C (1 g, 10 wt. %, wet) was added to the solution in one portion. The resulting mixture was stirred under hydrogen atmosphere (1 atm) at room temperature overnight. The solid was filtered off and the filtrate was concentrated to give the crude product. The crude was triturated with MTBE to give the desired product which was purified by HPLC (IF (2*25 cm. Sum), 60% MtBE/40% MeOH:DCM=1:1,80 bar, 20 ml/min) and corresponded to peak A @ 1.216 min/254 nm (1.13 g, 25%). [M+H]⁺=367.4.

### Step 5: (R)-3-(4-((R)-3,3-dimethyl-4-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carbonyl)pyrrolidin-1-yl)-2,6-difluorophenyl)piperidine-2,6-dione

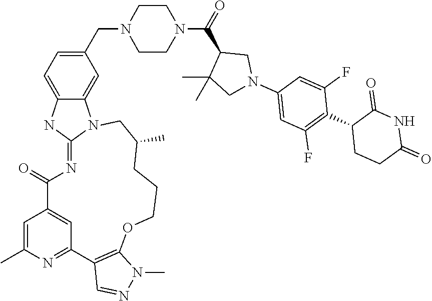 [SMILES: Cc1cc2cc(n1)-c1cnn(C)c1OCCC[C@@H](C)CN1/C(=N/C2=O)Nc2ccc(CN3CCN(C(=O)[C@H]4CN(c5cc(F)c([C@H]6CCC(=O)NC6=O)c(F)c5)CC4(C)C)CC3)cc21]

To a 40-mL microwave vial equipped with a magnetic stir bar was added (R,E)-11,26,7-trimethyl-56-(piperazin-1-ylmethyl)-52,53-dihydro-11H,51H-11-oxa-4-aza-5(2,1)-benzo[d]imidazola-2(2,4)-pyridina-1(4,5)-pyrazolacycloundecaphan-3-one (52.9 mg, 0.10 mmol), (R)-1-(4-((R)-2,6-dioxopiperidin-3-yl)-3,5-difluorophenyl)-4,4-dimethylpyrrolidine-3-carboxylic acid (36.6 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (62 mg, 71%). ¹H NMR (500 MHz, DMSO) δ 12.65 (s, 1H), 10.77 (s, 1H), 8.36 (s, 1H), 7.86 (s, 1H), 7.50 (s, 1H), 7.47 (s, 1H), 7.43 (d, J=8.0 Hz, 1H), 7.13 (d, J=8.0 Hz, 1H), 6.07 (d, J=12.5 Hz, 2H), 4.32-4.27 (m, 1H), 4.14 (d, J=11.5 Hz, 1H), 3.95-3.85 (m, 3H), 3.66 (s, 3H), 3.58-3.42 (m, 6H), 3.38-3.29 (m, 4H), 3.00 (dd, J=19.0, 9.0 Hz, 2H), 2.74-2.67 (m, 2H), 2.49 (s, 3H), 2.37-2.25 (m, 4H), 2.19-2.10 (m, 1H), 2.06-1.84 (m, 4H), 1.43-1.37 (m, 1H), 1.09 (s, 3H), 0.90 (s, 3H), 0.75 (d, J=6.5 Hz, 3H); [M+H]⁺=877.6.

### Example 78: (R)-3-(2,6-difluoro-4-((R)-3-(6-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-2,6-diazaspiro[3.3]heptane-2-carbonyl)pyrrolidin-1-yl)phenyl)piperidine-2,6-dione

### Step 1: tert-butyl (R,E)-6-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,6-diazaspiro[3.3]heptane-2-carboxylate

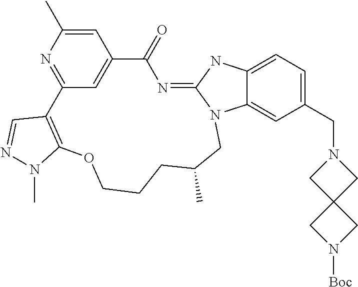 [SMILES: Cc1cc2cc(n1)-c1cnn(C)c1OCCC[C@@H](C)CN1/C(=N/C2=O)Nc2ccc(CN3CC4(C3)CN(C(=O)OC(C)(C)C)C4)cc21]

To a 40-mL microwave vial equipped with a magnetic stir bar was added (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (458 mg, 1.0 mmol), tert-butyl 2,6-diazaspiro[3.3]heptane-2-carboxylate (1.0 g, 5.0 mmol), NaBH(OAc)₃ (848 mg, 4.0 mmol) and DCM (20 mL) at room temperature. To the stirred reaction mixture was added AcOH (340 mL, 6.0 mmol). After stirring for 12 h at room temperature, the reaction mixture was diluted with sat. aq. NaHCO₃(30 mL) and the layers were separated. The aqueous layer was extracted with DCM (3×30 mL). The combined organic layers were washed with brine (50 mL), dried over anhydrous Na₂SO₄, filtered and concentrated under reduced pressure. Purification by flash column chromatography (0-10% MeOH in DCM) afforded the product (351 mg, 55%). [M+H]⁺=641.6.

### Step 2: (R,E)-5⁶-((2,6-diazaspiro[3.3]heptan-2-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

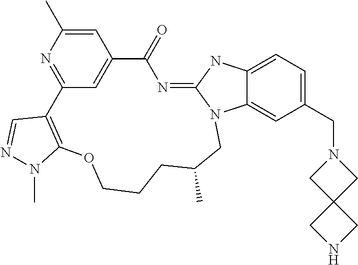 [SMILES: Cc1cc2cc(n1)-c1cnn(C)c1OCCC[C@@H](C)CN1/C(=N/C2=O)Nc2ccc(CN3CC4(CNC4)C3)cc21]

To a 100-mL round-bottomed flask equipped with a magnetic stir bar was added tert-butyl (R,E)-6-((1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,6-diazaspiro[3.3]heptane-2-carboxylate (351 mg, 0.55 mmol), DCM (20 mL) and TFA (5 mL). After stirring for 1 h at room temperature, the reaction mixture was concentrated under reduced pressure to afford the product as TFA salt (468 mg), which was used without further purification. [M+H]⁺=541.6.

### Step 3: (R)-3-(2,6-difluoro-4-((R)-3-(6-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-2,6-diazaspiro[3.3]heptane-2-carbonyl)pyrrolidin-1-yl)phenyl)piperidine-2,6-dione

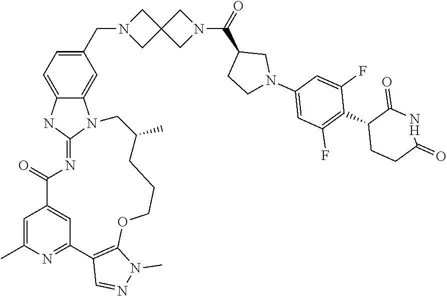 [SMILES: Cc1cc2cc(n1)-c1cnn(C)c1OCCC[C@@H](C)CN1/C(=N/C2=O)Nc2ccc(CN3CC4(C3)CN(C(=O)[C@@H]3CCN(c5cc(F)c([C@H]6CCC(=O)NC6=O)c(F)c5)C3)C4)cc21]

To a 40-mL microwave vial equipped with a magnetic stir bar was added (R,E)-56-((2,6-diazaspiro[3.3]heptan-2-yl)methyl)-11,26,7-trimethyl-52,53-dihydro-11H,51H-11-oxa-4-aza-5(2,1)-benzo[d]imidazola-2(2,4)-pyridina-1(4,5)-pyrazolacycloundecaphan-3-one (54.0 mg, 0.10 mmol), (R)-1-(4-((R)-2,6-dioxopiperidin-3-yl)-3,5-difluorophenyl)pyrrolidine-3-carboxylic acid (33.8 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water; acetonitrile=90:10-50:50 gradient elution) afforded the title compound (75 mg, 87%). ¹H NMR (500 MHz, DMSO) δ 12.63 (s, 1H), 10.77 (s, 1H), 8.36 (s, 1H), 7.86 (s, 1H), 7.50 (s, 1H), 7.41 (s, 1H), 7.40 (d, J=8.0 Hz, 1H), 7.06 (d, J=8.5 Hz, 1H), 6.14 (d, J=12.5 Hz, 2H), 4.32-4.21 (m, 3H), 4.13 (d, J=12.0 Hz, 1H), 3.96-3.93 (m, 2H), 3.90-3.85 (m, 3H), 3.66 (s, 3H), 3.58-3.53 (m, 2H), 3.37-3.30 (m, 5H), 3.16-3.12 (m, 3H), 3.07-3.01 (m, 1H), 2.78-2.66 (m, 2H), 2.49 (s, 3H), 2.21-2.10 (m, 1H), 2.08-1.82 (m, 7H), 1.43-1.35 (m, 1H), 0.75 (d, J=6.5 Hz, 3H); [M+H]⁺=861.5.

### Example 79: (R)-3-(2,6-difluoro-4-(3-(6-(((R,E)-1¹,2⁶,7-trimethyl-3-oxo-5²,5³-dihydro-1¹H,5¹H-11-oxa-4-aza-5(2, 1)-benzo[d]imidazola-2(2,4)-pyridina-1(4,5)-pyrazolacycloundecaphane-5⁶-yl)methyl)-2,6-diazaspiro[3.3]heptane-2-carbonyl)azetidin-1-yl)phenyl)piperidine-2,6-dione

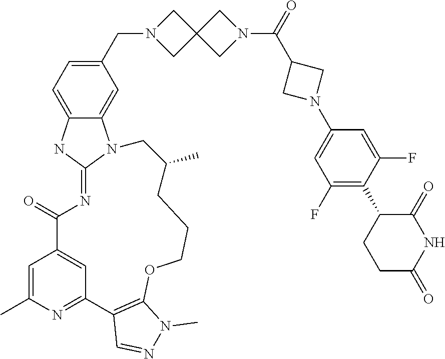 [SMILES: Cc1cc2cc(n1)-c1cnn(C)c1OCCC[C@@H](C)CN1/C(=N/C2=O)Nc2ccc(CN3CC4(C3)CN(C(=O)C3CN(c5cc(F)c([C@H]6CCC(=O)NC6=O)c(F)c5)C3)C4)cc21]

To a 40-mL microwave vial equipped with a magnetic stir bar was added (R,E)-5⁶-((2,6-diazaspiro[3.3]heptan-2-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (54.0 mg, 0.10 mmol), (R)-1-(4-(2,6-dioxopiperidin-3-yl)-3,5-difluorophenyl)azetidine-3-carboxylic acid (32.4 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous laver was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (53 mg, 63%). ¹H NMR (500 MHz, DMSO) δ 12.70 (s, 1H), 10.85 (s, 1H), 8.43 (s, 1H), 7.92 (s, 1H), 7.57 (s, 1H), 7.47 (s, 1H), 7.46 (d, J=8.0 Hz, 1H), 7.12 (d, J=8.5 Hz, 1H), 6.13 (d, J=11.5 Hz, 2H), 4.38-4.34 (m, 1H), 4.20-4.17 (m, 3H), 4.04-3.92 (m, 8H), 3.80 (t, J=7.0 Hz, 2H), 3.73 (s, 3H), 3.64-3.59 (m, 2H), 3.55-3.49 (m, 2H), 3.33-3.28 (m, 2H), 2.83-2.73 (m, 2H), 2.56 (s, 3H), 2.27-2.16 (m, 1H), 2.10-1.86 (m, 5H), 1.50-1.43 (m, 1H), 0.82 (d, J=6.5 Hz, 3H); [M+H]⁺=847.5.

### Example 80: (R)-3-(4-((R)-3,3-dimethyl-4-(6-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,6-diazaspiro[3.3]heptane-2-carbonyl)pyrrolidin-1-yl)-2,6-difluorophenyl)piperidine-2,6-dione

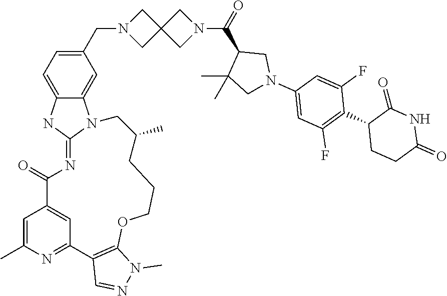 [SMILES: Cc1cc2cc(n1)-c1cnn(C)c1OCCC[C@@H](C)CN1/C(=N/C2=O)Nc2ccc(CN3CC4(C3)CN(C(=O)[C@H]3CN(c5cc(F)c([C@H]6CCC(=O)NC6=O)c(F)c5)CC3(C)C)C4)cc21]

To a 40-mL microwave vial equipped with a magnetic stir bar was added (R,E)-5⁶-((2,6-diazaspiro[3.3]heptan-2-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (54.0 mg, 0.10 mmol), (R)-1-(4-((R)-2,6-dioxopiperidin-3-yl)-3,5-difluorophenyl)-4,4-dimethylpyrrolidine-3-carboxylic acid (36.6 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10˜ 50:50 gradient elution) afforded the title compound (51 mg, 57%). ¹H NMR (500 MHz, DMSO) δ 12.63 (s, 1H), 10.76 (s, 1H), 8.36 (s, 1H), 7.86 (s, 1H), 7.50 (s, 1H), 7.40-7.39 (m, 2H), 7.06 (d, J=8.0 Hz, 1H), 6.07 (d, J=12.5 Hz, 2H), 4.31-4.25 (m, 2H), 4.17-4.11 (m, 2H), 3.95-3.85 (m, 5H), 3.67 (s, 3H), 3.55 (s, 2H), 3.35-3.21 (m, 6H), 3.04-2.96 (m, 2H), 2.77-2.67 (m, 3H), 2.49 (s, 3H), 2.21-2.12 (m, 1H), 2.06-1.85 (m, 5H), 1.45-1.35 (m, 1H), 1.07 (s, 3H), 0.91 (s, 3H), 0.75 (d, J=6.5 Hz, 3H); [M+H]⁺=889.6.

### Example 81: (R)-3-(2,6-difluoro-4-(2-(6-(((R,E)-1¹,2⁶,7-trimethyl-3-oxo-5²,53-dihydro-1¹H,5¹H-1l-oxa-4-aza-5(2,1)-benzo[d]imidazola-2(2,4)-pyridina-1(4,5)-pyrazolacycloundecaphane-5⁶-yl)methyl)-2,6-diazaspiro[3.3]heptan-2-yl)ethyl)phenyl)piperidine-2,6-dione

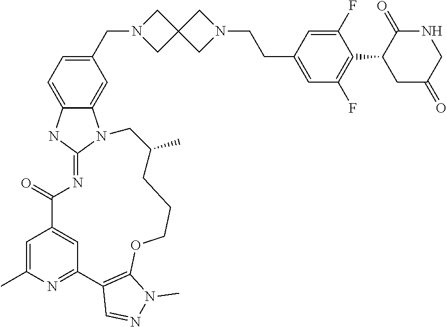 [SMILES: Cc1cc2cc(n1)-c1cnn(C)c1OCCC[C@@H](C)CN1/C(=N/C2=O)Nc2ccc(CN3CC4(CN(CCc5cc(F)c([C@H]6CC(=O)CNC6=O)c(F)c5)C4)C3)cc21]

To a 40-mL microwave vial equipped with a magnetic stir bar was added (R,E)-5⁶-((2,6-diazaspiro[3.3]heptan-2-yl)methyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (54.0 mg, 0.10 mmol), (R)-2-(4-(2,6-dioxopiperidin-3-yl)-3,5-difluorophenyl)acetaldehyde (58.7 mg, 0.22 mmol), DMSO (2 mL) and DCE (8 mL). To the reaction mixture was added NaBH(OAc)₃ (63.6 mg, 0.3 mmol) and 6 drops of AcOH. After stirring for 4 h at 70° C., the reaction mixture was diluted with sat. aq. NaHCO₃(20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by prep-TLC followed by prep-HPLC chromatography (0.1% FA in water; acetonitrile=90:10-50:50 gradient elution) afforded the title compound (30 mg, 38%). ¹H NMR (500 MHz, DMSO) δ 12.69 (s, 1H), 10.94 (s, 1H), 8.42 (s, 1H), 7.92 (s, 1H), 7.56 (s, 1H), 7.46 (s, 1H), 7.45 (d, J=8.0 Hz, 1H), 7.11 (d, J=8.5 Hz, 1H), 6.97 (d, J=10.0 Hz, 2H), 4.42-4.32 (m, 1H), 4.22-4.13 (m, 2H), 4.02-3.90 (m, 3H), 3.73 (s, 3H), 3.61 (s, 2H), 3.25-3.18 (m, 8H), 2.85-2.75 (m, 3H), 2.56 (s, 3H), 2.56-2.53 (m, 3H), 2.26-2.16 (m, 1H), 2.07-2.03 (m, 1H), 2.02-1.89 (m, 3H), 1.51-1.42 (m, 1H), 0.81 (d, J=6.5 Hz, 3H); [M+H]*=792.6.

### Example 82: (R)-3-(2,6-difluoro-4-((R)-3-((1S,4S)-5-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl )-2,5-diazabicyclo[2.2.1]heptane-2-carbonyl)pyrrolidin-1-yl)phenyl)piperidine-2,6-dione

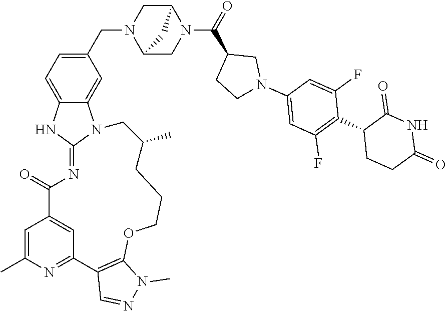 [SMILES: Cc1cc2cc(n1)-c1cnn(C)c1OCCC[C@@H](C)CN1/C(=N/C2=O)Nc2ccc(CN3C[C@@H]4C[C@H]3CN4C(=O)[C@@H]3CCN(c4cc(F)c([C@H]5CCC(=O)NC5=O)c(F)c4)C3)cc21]

The titled compound was prepared in a manner similar to that in Example 5. ¹H NMR (500 MHz, DMSO) δ_{H} 12.70 (s, 1H), 10.84 (s, 1H), 8.43 (s, 1H), 7.92 (s, 1H), 7.56 (d, J=7.2 Hz, 2H), 7.48 (d, J=8.1 Hz, 1H), 7.23 (d, J=8.2 Hz, 1H), 6.22 (dd, J=12.2, 6.4 Hz, 2H), 4.59 (d, J=28.4 Hz, 1H), 4.36 (s, 1H), 4.25-4.15 (m, 1H), 4.05-3.91 (m, 3H), 3.82 (d, J=7.9 Hz, 2H), 3.73 (s, 3H), 3.58-3.41 (m, 4H), 3.28 (d, J=6.1 Hz, 4H), 2.90-2.74 (m, 3H), 2.56 (s, 4H), 2.48 (s, 1H), 2.30-2.16 (m, 2H), 2.14-1.82 (m, 6H), 1.69 (dd, J=35.7, 8.9 Hz, 1H), 1.47 (s, 1H), 0.82 (d, J=6.4 Hz, 3H). [M+H]⁺=861.7.

### Example 83: (3R)-3-(2,6-difluoro-4-((3R)-3-(3-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,8-di azabicyclo[3.2.1]octane-8-carbonyl)pyrrolidin-1-yl)phenyl)piperidine-2,6-dione

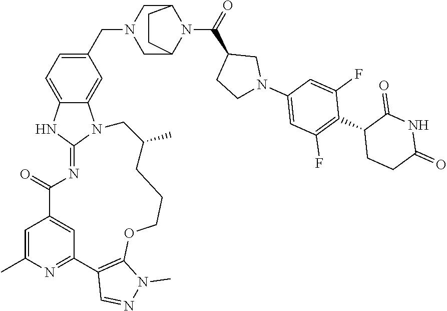 [SMILES: Cc1cc2cc(n1)-c1cnn(C)c1OCCC[C@@H](C)CN1/C(=N/C2=O)Nc2ccc(CN3CC4CCC(C3)N4C(=O)[C@@H]3CCN(c4cc(F)c([C@H]5CCC(=O)NC5=O)c(F)c4)C3)cc21]

The titled compound was prepared in a manner similar to that in Example 5. ¹H NMR (500 MHz, DMSO) δ_{H} 12.71 (s, 1H), 10.84 (d, J=5.0 Hz, 1H), 8.43 (s, 1H), 7.92 (s, 1H), 7.57 (s, 1H), 7.53-7.47 (m, 2H), 7.19 (t, J=7.6 Hz, 1H), 6.22 (dd, J=12.1, 6.9 Hz, 2H), 4.38 (s, 3H), 4.22 (d, J=12.7 Hz, 1H), 4.05-3.97 (m, 2H), 3.92 (s, 1H), 3.73 (s, 3H), 3.64 (d, J=11.4 Hz, 1H), 3.56 (d, J=11.0 Hz, 1H), 3.46-3.40 (m, 2H), 3.25 (d, J=11.8 Hz, 2H), 2.80-2.60 (m, 4H), 2.55 (d, J=8.0 Hz, 3H), 2.49-2.45 (m, 1H), 2.25-1.85 (m, 13H), 1.69 (d, J=5.2 Hz, 1H), 1.46 (s, 1H), 0.83 (d, J=6.4 Hz, 3H). [M+H]⁺=875.7.

### Example 84: (R)-3-(2,6-difluoro-4-((R)-3-(2-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-2,7-diazaspiro[3.5]nonane-7-carbonyl)pyrrolidin-1-yl)phenyl)piperidine-2,6-dione

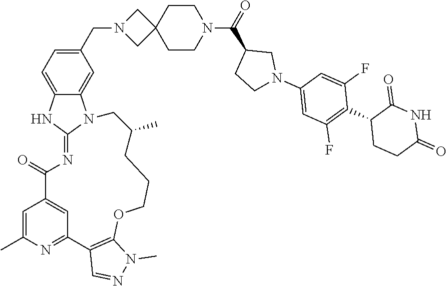 [SMILES: Cc1cc2cc(n1)-c1cnn(C)c1OCCC[C@@H](C)CN1/C(=N/C2=O)Nc2ccc(CN3CC4(CCN(C(=O)[C@@H]5CCN(c6cc(F)c([C@H]7CCC(=O)NC7=O)c(F)c6)C5)CC4)C3)cc21]

The titled compound was prepared in a manner similar to that in Example 5. ¹H NMR (500 MHz, DMSO) δ_{H} 12.70 (s, 1H), 10.84 (s, 1H), 8.43 (s, 1H), 7.92 (s, 1H), 7.57 (s, 1H), 7.51-7.45 (m, 2H), 7.16 (d, J=8.2 Hz, 1H), 6.20 (d, J=12.2 Hz, 2H), 4.36 (d, J=4.6 Hz, 1H), 4.20 (d, J=11.5 Hz, 1H), 4.05-3.89 (m, 3H), 3.72 (d, J=13.8 Hz, 5H), 3.52-3.40 (m, 7H), 3.26-3.22 (m, 2H), 3.02 (s, 4H), 2.84-2.72 (m, 2H), 2.56 (s, 3H), 2.48 (s, 1H), 2.21 (s, 1H), 2.16-1.88 (m, 6H), 1.72 (d, J=5.1 Hz, 2H), 1.62 (s, 2H), 1.47 (d, J=6.2 Hz, 1H), 0.82 (d, J=6.4 Hz, 3H). [M+H]⁺=889.7.

### Example 85: 2-(2,6-dioxopiperidin-3-yl)-5-((S)-3-((4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)methyl)pyrrolidin-1-yl)isoindoline-1,3-dione

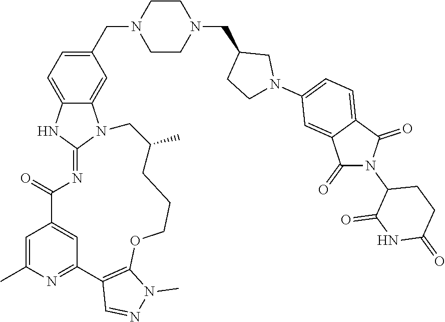 [SMILES: Cc1cc2cc(n1)-c1cnn(C)c1OCCC[C@@H](C)CN1/C(=N/C2=O)Nc2ccc(CN3CCN(C[C@@H]4CCN(c5ccc6c(c5)C(=O)N(C5CCC(=O)NC5=O)C6=O)C4)CC3)cc21]

The titled compound was prepared in a manner similar to that in Example 14.

¹H NMR (500 MHz, DMSO) δ 12.70 (s, 1H), 11.06 (s, 1H), 8.43 (s, 1H), 7.92 (s, 1H), 7.63 (d, J=8.4 Hz, 1H), 7.57 (s, 1H), 7.52-7.45 (m, 2H), 7.17 (d, J=8.3 Hz, 1H), 6.88 (s, 1H), 6.80 (d, J=8.2 Hz, 1H), 5.05 (dd, J=12.9, 5.0 Hz, 1H), 4.36 (s, 1H), 4.22-4.18 (m, 1H), 4.00 (s, 1H), 3.96-3.89 (m, 1H), 3.73 (s, 3H), 3.62-3.45 (m, 4H), 3.41-3.36 (m, 1H), 3.16-3.08 (m, 1H), 2.94-2.75 (m, 2H), 2.65-2.53 (m, 7H), 2.48-2.31 (m, 9H), 2.26-2.17 (m, 1H), 2.15-2.06 (m, 1H), 2.05-1.87 (m, 3H), 1.78-1.68 (m, 1H), 1.52-1.40 (m, 1H), 0.81 (d, J=6.3 Hz, 3H); [M+H]⁺=868.60.

### Example 86: 2-(2,6-dioxopiperidin-3-yl)-5-((R)-3-((4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)methyl)pyrrolidin-1-yl)isoindoline-1,3-dione

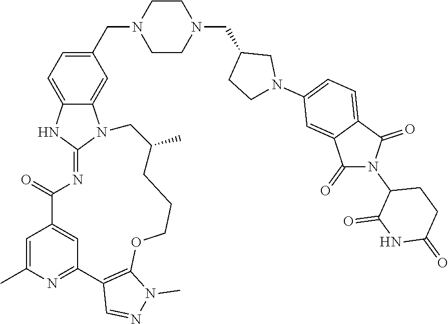 [SMILES: Cc1cc2cc(n1)-c1cnn(C)c1OCCC[C@@H](C)CN1/C(=N/C2=O)Nc2ccc(CN3CCN(C[C@H]4CCN(c5ccc6c(c5)C(=O)N(C5CCC(=O)NC5=O)C6=O)C4)CC3)cc21]

The titled compound was prepared in a manner similar to that in Example 14.

¹H NMR (500 MHz, DMSO) δ 12.70 (s, 1H), 11.06 (s, 1H), 8.43 (s, 1H), 7.92 (s, 1H), 7.63 (d, J=8.4 Hz, 1H), 7.57 (s, 1H), 7.52-7.45 (m, 2H), 7.17 (d, J=8.3 Hz, 1H), 6.88 (s, 1H), 6.80 (d, J=8.2 Hz, 1H), 5.05 (dd, J=12.9, 5.0 Hz, 1H), 4.36 (s, 1H), 4.22-4.18 (m, 1H), 4.00 (s, 1H), 3.96-3.89 (m, 1H), 3.73 (s, 3H), 3.62-3.45 (m, 4H), 3.41-3.36 (m, 1H), 3.16-3.08 (m, 1H), 2.94-2.75 (m, 2H), 2.65-2.53 (m, 7H), 2.48-2.31 (m, 9H), 2.26-2.17 (m, 1H), 2.15-2.06 (m, 1H), 2.05-1.87 (m, 3H), 1.78-1.68 (m, 1H), 1.52-1.40 (m, 1H), 0.81 (d, J=6.3 Hz, 3H); [M+H]⁺=868.60.

### Example 88: (R)-3-(2,6-difluoro-4-((S)-3-((4-(((R,E)-1¹,2⁶,7-trimethyl-3-oxo-5²,5³-dihydro-1¹H,5¹H-1l-oxa-4-aza -5(2,1)-benzo[d]imidazola-2(2,4)-pyridina-1(4,5)-pyrazolacycloundecaphane-5⁶-yl)methyl)piperazin -1-yl)methyl)pyrrolidin-1-yl)phenyl)piperidine-2,6-dione

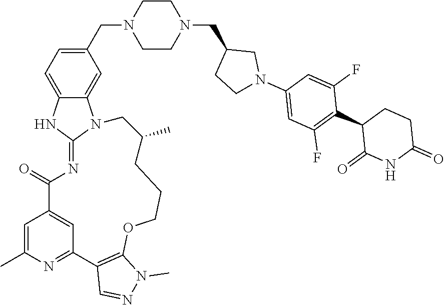 [SMILES: Cc1cc2cc(n1)-c1cnn(C)c1OCCC[C@@H](C)CN1/C(=N/C2=O)Nc2ccc(CN3CCN(C[C@@H]4CCN(c5cc(F)c([C@H]6CCC(=O)NC6=O)c(F)c5)C4)CC3)cc21]

The titled compound was prepared in a manner similar to that in Example 14.

¹H NMR (500 MHz, DMSO) δ 12.70 (s, 1H), 10.83 (s, 1H), 8.43 (s, 1H), 7.92 (s, 1H), 7.56 (s, 1H), 7.52-7.45 (m, 2H), 7.17 (d, J=8.1 Hz, 1H), 6.17 (d, J=9.6 Hz, 2H), 4.40-4.32 (m, 1H), 4.20 (d, J=11.8 Hz, 1H), 4.00 (dd, J=11.1, 4.6 Hz, 2H), 3.96-3.90 (m, 1H), 3.73 (s, 3H), 3.57 (s, 2H), 3.30-3.23 (m, 1H), 3.22-3.13 (m, 1H), 2.97-2.89 (m, 1H), 2.84-2.71 (m, 2H), 2.61-2.52 (m, 5H), 2.48-2.34 (m, 9H), 2.30 (d, J=7.4 Hz, 2H), 2.27-2.16 (m, 1H), 2.12-1.88 (m, 5H), 1.73-1.62 (m, 1H), 1.52-1.42 (m, 1H), 0.81 (d, J=6.3 Hz, 3H); [M+H]⁺=835.60.

### Example 90: (R)-3-(2,6-difluoro-4-((R)-3-((4-(((R,E)-1¹,2⁶,7-trimethyl-3-oxo-5²,5³-dihydro-¹H,5¹H-11-oxa-4-aza-5(2,1)-benzo[d]imidazola-2(2,4)-pyridina-1(4,5)-pyrazolacycloundecaphane-5⁶-yl)methyl )piperazin-1-yl)methyl)pyrrolidin-1-yl)phenyl)piperidine-2,6-dione

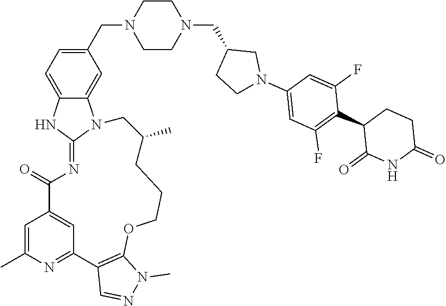 [SMILES: Cc1cc2cc(n1)-c1cnn(C)c1OCCC[C@@H](C)CN1/C(=N/C2=O)Nc2ccc(CN3CCN(C[C@H]4CCN(c5cc(F)c([C@H]6CCC(=O)NC6=O)c(F)c5)C4)CC3)cc21]

The titled compound was prepared in a manner similar to that in Example 14.

¹H NMR (500 MHz, DMSO) δ 12.70 (s, 1H), 10.83 (s, 1H), 8.43 (s, 1H), 7.92 (s, 1H), 7.56 (s, 1H), 7.52-7.45 (m, 2H), 7.17 (d, J=8.1 Hz, 1H), 6.17 (d, J=9.6 Hz, 2H), 4.40-4.32 (m, 1H), 4.20 (d, J=11.8 Hz, 1H), 4.00 (dd, J=11.1, 4.6 Hz, 2H), 3.96-3.90 (m, 1H), 3.73 (s, 3H), 3.57 (s, 2H), 3.30-3.23 (m, 1H), 3.22-3.13 (m, 1H), 2.97-2.89 (m, 1H), 2.84-2.71 (m, 2H), 2.61-2.52 (m, 5H), 2.48-2.34 (m, 9H), 2.30 (d, J=7.4 Hz, 2H), 2.27-2.16 (m, 1H), 2.12-1.88 (m, 5H), 1.73-1.62 (m, 1H), 1.52-1.42 (m, 1H), 0.81 (d, J=6.3 Hz, 3H); [M+H]⁺=835.60.

### Example 91: (R)-3-(2,6-difluoro-4-(2-((1S,4S)-5-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,5-diazabicyclo[2.2.1]heptan-2-yl)ethyl)phenyl)piperidine-2,6-dione

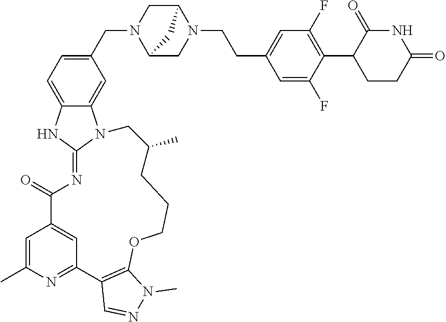 [SMILES: Cc1cc2cc(n1)-c1cnn(C)c1OCCC[C@@H](C)CN1/C(=N/C2=O)Nc2ccc(CN3C[C@@H]4C[C@H]3CN4CCc3cc(F)c(C4CCC(=O)NC4=O)c(F)c3)cc21]

The titled compound was prepared in a manner similar to that in Example 14. ¹H NMR (500 MHz, DMSO) δ_{H} 12.68 (s, 1H), 10.95 (s, 1H), 8.42 (s, 1H), 7.92 (s, 1H), 7.55 (d, J=10.9 Hz, 2H), 7.47 (d, J=8.1 Hz, 1H), 7.21 (d, J=8.1 Hz, 1H), 7.03 (d, J=10.2 Hz, 2H), 4.36 (d, J=4.4 Hz, 1H), 4.24-4.14 (m, 2H), 4.00 (s, 1H), 3.97-3.89 (m, 1H), 3.81 (d, J=13.4 Hz, 1H), 3.72 (d, J=10.1 Hz, 4H), 3.65-3.61 (m, 1H), 3.29 (s, 4H), 2.80 (t, J=13.9 Hz, 4H), 2.70-2.65 (m, 4H), 2.56 (s, 3H), 2.22-2.08 (m, 2H), 2.03-1.88 (m, 3H), 1.64 (dd, J=34.3, 8.8 Hz, 2H), 1.46 (s, 1H), 0.81 (d, J=6.3 Hz, 3H). [M+H]⁺=792.7.

### Example 92: (3R)-3-(2,6-difluoro-4-(2-(3-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,8-diazabicyclo[3.2.1]octan-8-yl)ethyl)phenyl)piperidine-2,6-dione

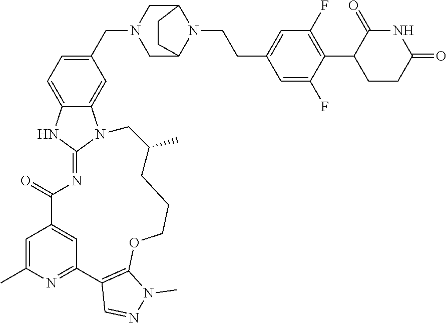 [SMILES: Cc1cc2cc(n1)-c1cnn(C)c1OCCC[C@@H](C)CN1/C(=N/C2=O)Nc2ccc(CN3CC4CCC(C3)N4CCc3cc(F)c(C4CCC(=O)NC4=O)c(F)c3)cc21]

The titled compound was prepared in a manner similar to that in Example 14. ¹H NMR (500 MHz, DMSO) δ_{H} 12.70 (s, 1H), 10.94 (s, 1H), 8.42 (s, 1H), 7.92 (s, 1H), 7.57 (s, 1H), 7.48 (d, J=8.3 Hz, 2H), 7.16 (d, J=8.1 Hz, 1H), 7.03 (d, J=10.2 Hz, 2H), 4.36 (d, J=4.5 Hz, 1H), 4.20 (d, J=3.0 Hz, 2H), 4.00 (s, 1H), 3.93-3.86 (m, 1H), 3.73 (s, 3H), 3.59 (s, 1H), 3.51 (s, 1H), 3.27-3.22 (m, 2H), 2.85-2.69 (m, 4H), 2.60-2.53 (m, 7H), 2.30 (d, J=9.7 Hz, 1H), 2.22 (s, 2H), 2.16-2.07 (m, 1H), 2.00-1.72 (m, 8H), 1.46 (s, 1H), 0.82 (d, J=6.4 Hz, 3H). [M+H]⁺=806.7.

### Example 93: (R)-3-(2,6-difluoro-4-(2-(2-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,7-diazaspiro[3.5]nonan-7-yl)ethyl)phenyl)piperidine-2,6-dione

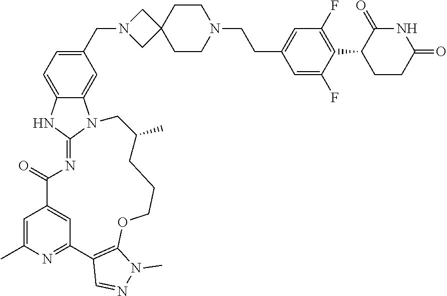 [SMILES: Cc1cc2cc(n1)-c1cnn(C)c1OCCC[C@@H](C)CN1/C(=N/C2=O)Nc2ccc(CN3CC4(CCN(CCc5cc(F)c([C@H]6CCC(=O)NC6=O)c(F)c5)CC4)C3)cc21]

The titled compound was prepared in a manner similar to that in Example 14. ¹H NMR (500 MHz, DMSO) δ_{H} 12.68 (s, 1H), 10.94 (s, 1H), 8.42 (s, 1H), 7.92 (s, 1H), 7.56 (s, 1H), 7.47 (d, J=11.2 Hz, 2H), 7.14 (d, J=8.0 Hz, 1H), 6.99 (d, J=10.2 Hz, 2H), 4.36 (d, J=4.2 Hz, 1H), 4.18 (dd, J=11.6, 6.5 Hz, 2H), 4.02-3.90 (m, 2H), 3.73 (s, 3H), 3.67 (s, 2H), 2.95 (s, 4H), 2.83-2.70 (m, 4H), 2.58-2.53 (m, 4H), 2.48-2.44 (m, 2H), 2.38-2.20 (m, 5H), 2.11 (d, J=10.3 Hz, 1H), 2.03-1.87 (m, 3H), 1.66 (s, 4H), 1.46 (s, 1H), 0.81 (d, J=6.3 Hz, 3H). [M+H]⁺=820.7.

### Example 94: 3-(7-fluoro-3-methyl-2-oxo-4-(3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piper azine-1-carbonyl)azetidin-1-yl)-2,3-dihydro-1H-benzo[d]imidazol-1-yl)piperidine-2,6-dione

### Step 1: 2-((2,6-bis(benzyloxy)pyridin-3-yl) amino)-6-bromo-3-fluorobenzoic acid

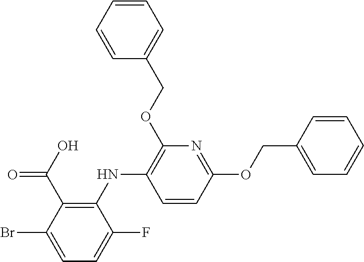 [SMILES: O=C(O)c1c(Br)ccc(F)c1Nc1ccc(OCc2ccccc2)nc1OCc1ccccc1]

To a solution of 2,6-bis(benzyloxy)pyridin-3-amine (15 g, 49.0 mmol) in THF (300 mL) was added LiHMDS (1 M in THF, 80 mL) dropwise in 30 min at 0° C., the reaction solution was stirred for 60 min at this temperature, then to this was added 6-bromo-2,3-difluorobenzoic acid (10 g, 42.4 mmol) in THF (50 mL) dropwise in 20 min. The resulting solution was stirred for 12 h at 10-20° C. The reaction was quenched by the addition of sat. aq. NH₄Cl solution. Concentrated. The residue was purified by a silica gel column, eluted with DCM/MeOH to afford product (20 g, 90.4%). [M+H]⁺=523.1.

### Step 2: 1-(2,6-bis(benzyloxy))pyridin-3-yl)-4-bromo-7-fluoro-1,3-dihydro-2H-benzo[d]imidazol-2-one

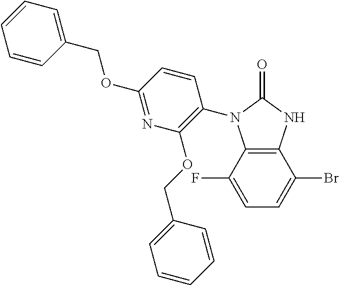 [SMILES: O=c1[nH]c2c(Br)ccc(F)c2n1-c1ccc(OCc2ccccc2)nc1OCc1ccccc1]

To a solution of 2-((2,6-bis(benzyloxy)pyridin-3-yl) amino)-6-bromo-3-fluorobenzoic acid (20 g, 38.3 mmol) in DMA (400 mL) were added TEA (11.6 g, 114.8 mmol) and DPPA (15.8 g, 57.4 mmol). The mixture was stirred for 12 h at 80° C. under nitrogen atmosphere. The mixture was quenched by water at 30° C. and was extracted with EtOAc. The combined organic phases were concentrated under reduced pressure. The residue was purified by a silica gel column, eluted with DCM/EtOAc to afford product (18 g, 90.9%). [M+H]⁺=520.2.

### Step 3: 1-(2,6-bis(benzyloxy)pyridin-3-yl)-4-bromo-7-fluoro-3-methyl-1,3-dihydro-2H-benzo[d]imidazol-2-one

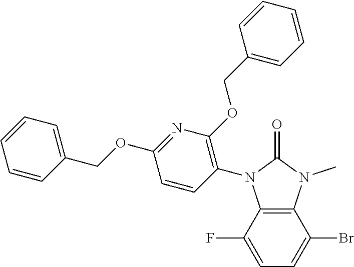 [SMILES: Cn1c(=O)n(-c2ccc(OCc3ccccc3)nc2OCc2ccccc2)c2c(F)ccc(Br)c21]

To a solution of 142,6-bis(benzyloxy)pyridin-3-yl)-4-bromo-7-fluoro-1,3-dihydro-2H-benzo[d]imidazol-2-one (10 g, 19.2 mmol) in DMF (100 mL) was added Cs₂CO₃(18.7 g, 57.5 mmol) at 0° C. for 10 min. Then to this was added CH₃I (8.2 g, 57.7 mmol). The resulting solution was stirred for 12 h at 10-20° C. The mixture was quenched by water at 30° C. and was extracted with EtOAc. The combined organic phases were concentrated under reduced pressure. The residue was purified by a silica gel column, eluted with DCM/EtOAc to afford product (8 g, 77.9%). [M+H]⁺=534.3.

### Step 4: benzyl 1-(1-(2,6-bis(benzyloxy)pyridin-3-yl)-7-fluoro-3-methyl-2-oxo-2,3-dihydro-1H-benzo[d]imidazol-4-yl) azetidine-3-carboxylate

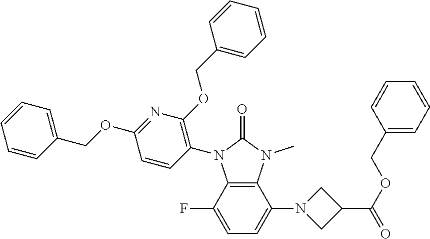 [SMILES: Cn1c(=O)n(-c2ccc(OCc3ccccc3)nc2OCc2ccccc2)c2c(F)ccc(N3CC(C(=O)OCc4ccccc4)C3)c21]

To a solution of 1-(2,6-bis(benzyloxy)pyridin-3-yl)-4-bromo-7-fluoro-3-methyl-1,3-dihydro-2H-benzo[d]imidazol-2-one (4 g, 7.4 mmol) in dioxane (40 mL) were added benzyl azetidine-3-carboxylate TFA salt (3.0 g, 10.4 mmol), Ruphos (0.6 g, 1.2 mmol), Pd₂(dba)₃ (0.6 g, 0.6 mmol) and Cs₂CO₃(9.6 g, 29.6 mmol). The resulting solution was stirred for 3 h at 90° C. under nitrogen atmosphere. The resulting mixture was concentrated under reduced pressure. The residue was purified by a silica gel column, eluted with DCM/EtOAc to afford product (2.36 g, 49.5%). [M+H]⁺=645.2.

### Step 5: 1-(1-(2,6-dioxopiperidin-3-yl)-7-fluoro-3-methyl-2-oxo-2,3-dihydro-1H-benzo[d]imidazol-4-yl) azetidine-3-carboxylic acid

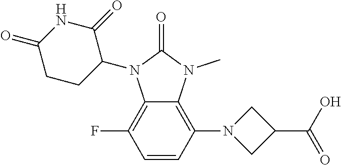 [SMILES: Cn1c(=O)n(C2CCC(=O)NC2=O)c2c(F)ccc(N3CC(C(=O)O)C3)c21]

To a solution of benzyl 1-(1-(2,6-bis(benzyloxy)pyridin-3-yl)-7-fluoro-3-methyl-2-oxo-2,3-dihydro-1H-benzo[d]imidazol-4-yl) azetidine-3-carboxylate (1.4 g, 2.2 mmol) in THF (30 mL) were added Pd/C (1.4 g, 10% wt) and CH₃COOH (0.3 mL). The resulting solution was stirred for 8 h at 30° C. under H₂ atmosphere. After filtration, the filtrate was concentrated under reduced pressure. This was resulted in 1-(1-(2,6-dioxopiperidin-3-yl)-7-fluoro-3-methyl-2-oxo-2,3-dihydro-1H-benzo[d]imidazol-4-yl) azetidine-3-carboxylic acid (0.5 g, 65.6%). ¹H NMR (300 MHz, DMSO-d₆) δ 12.48 (s, 1H), 11.10 (d, J=8.4 Hz, 1H), 7.30-7.05 (m, 1H), 6.98-6.79 (m, 1H), 5.49 (s, 1H), 4.08-3.91 (m, 1H), 3.85 (td, J=6.6, 2.7 Hz, 1H), 3.53 (s, 2H), 3.50-3.35 (m, 3H), 2.97 (d, J=14.8 Hz, 1H), 2.61 (d, J=18.1 Hz, 1H), 2.38-2.02 (m, 2H), 1.95 (d, J=23.9 Hz, 1H). [M+H]⁺=377.1.

### Step 6: 3-(7-fluoro-3-methyl-2-oxo-4-(3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piper azine-1-carbonyl)azetidin-1-yl)-2,3-dihydro-1H-benzo[d]imidazol-1-yl)piperidine-2,6-dione

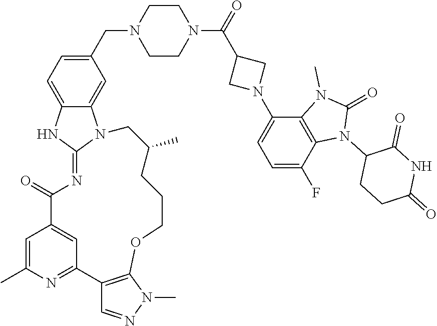 [SMILES: Cc1cc2cc(n1)-c1cnn(C)c1OCCC[C@@H](C)CN1/C(=N/C2=O)Nc2ccc(CN3CCN(C(=O)C4CN(c5ccc(F)c6c5n(C)c(=O)n6C5CCC(=O)NC5=O)C4)CC3)cc21]

To a 40-mL microwave vial equipped with a magnetic stir bar was added (R,E)-1¹,2⁶,7-trimethyl-5⁶-(piperazin-1-ylmethyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (52.9 mg, 0.10 mmol), 1-(1-(2,6-dioxopiperidin-3-yl)-7-fluoro-3-methyl-2-oxo-2,3-dihydro-1H-benzo[d]imidazol-4-yl)azetidine-3-carboxylic acid (37.6 mg, 0.10 mmol) and DCM (8 mL). To the reaction mixture was added N,N-diethylpropylethylamine (174 mL, 1.0 mmol) and T3P (50 wt %, 127 mg, 0.2 mmol). After stirring for 1.5 h at room temperature, the reaction mixture was diluted with brine (30 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10˜ 50:50 gradient elution) afforded the title compound (58 mg, 65%). ¹H NMR (500 MHz, DMSO) δ 12.71 (s, 1H), 11.89 (s, 1H), 8.43 (s, 1H), 7.92 (s, 1H), 7.57 (s, 1H), 7.52 (s, 1H), 7.49 (d, J=8.0 Hz, 1H), 7.19 (d, J=8.0 Hz, 1H), 6.87 (t, J=9.5 Hz, 1H), 6.77 (dd, J=8.5, 4.0 Hz, 1H), 5.51-5.46 (m, 1H), 4.38-4.34 (m, 1H), 4.20 (d, J=12.0 Hz, 1H), 4.02-3.91 (m, 5H), 3.88-3.86 (m, 3H), 3.73 (s, 3H), 3.65-3.61 (m, 5H), 3.47 (s, 3H), 3.05-2.92 (m, 1H), 2.56 (s, 3H), 2.42-2.34 (m, 4H), 2.28-2.16 (m, 2H), 2.06-1.80 (m, 4H), 1.52-1.41 (m, 2H), 0.81 (d, J=6.5 Hz, 3H); [M+H]⁺=887.5.

### Example 96: (R)-3-(2,6-difluoro-4-((R)-3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazine-1-carbonyl)pyrrolidin-1-yl)phenyl)piperidine-2,6-dione

### Step 1: (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxylic acid

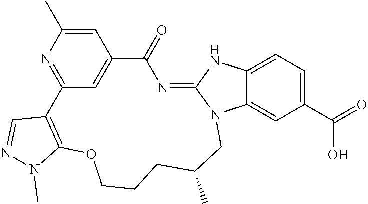 [SMILES: Cc1cc2cc(n1)-c1cnn(C)c1OCCC[C@@H](C)CN1/C(=N/C2=O)Nc2ccc(C(=O)O)cc21]

To a 40-mL microwave vial equipped with a magnetic stir bar was added (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbaldehyde (458 mg, 1.0 mmol), NaClO₂ (362 mg, 4.0 mmol), NaH₂PO₄ (960 mg, 8.0 mmol) and THF/tBuOH/H₂O (4:4:1, 18 mL) at room temperature. After stirring for 3 h at room temperature, the reaction mixture was filtered through celite and the solid was washed with MeOH (40 mL). The resulting solution was dried over anhydrous Na₂SO₄, filtered and concentrated under reduced pressure to afford the desired product (307 mg, 65%), which was used without further purification. [M+H]⁺=475.3.

### Step 2: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazine-1-carboxylate

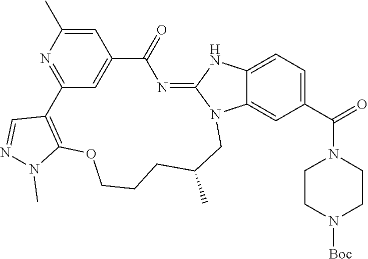 [SMILES: Cc1cc2cc(n1)-c1cnn(C)c1OCCC[C@@H](C)CN1/C(=N/C2=O)Nc2ccc(C(=O)N3CCN(C(=O)OC(C)(C)C)CC3)cc21]

To a 40-mL microwave vial equipped with a magnetic stir bar was added (R,E)-1 ,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carboxylic acid (307 mg, 0.65 mmol), tert-butyl piperazine-1-carboxylate (241 mg, 1.3 mmol) and DCM (10 mL) at room temperature. To the reaction mixture was added N,N-diethylpropylethylamine (906 mL, 5.2 mmol) and T3P (50 wt %, 827 mg, 1.3 mmol). After stirring for 2 h at room temperature, the reaction mixture was diluted with brine (30 mL) and the layers were separated. The aqueous layer was extracted with DCM (3×30 mL). The combined organic layers were washed with brine (50 mL), dried over anhydrous Na₂SO₄, filtered and concentrated under reduced pressure. Purification by flash column chromatography (0-10% MeOH in DCM) afforded the product (353 mg, 85%). [M+H]⁺=643.4.

### Step 3: (R,E)-1¹,2⁶,7-trimethyl-5⁶-(piperazine-1-carbonyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

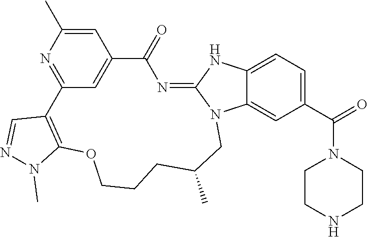 [SMILES: Cc1cc2cc(n1)-c1cnn(C)c1OCCC[C@@H](C)CN1/C(=N/C2=O)Nc2ccc(C(=O)N3CCNCC3)cc21]

To a 100-mL round-bottomed flask equipped with a magnetic stir bar was added tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazine-1-carboxylate (353 mg, 0.53 mmol), DCM (10 mL) and TFA (2 mL). After stirring for 1 h at room temperature, the reaction mixture was concentrated under reduced pressure. To the residue was added DCM (30 mL) and sat. aq. NaHCO₃ (20 mL), and the layers were separated. The aqueous layer was extracted with DCM (3×30 mL). The combined organic layers were washed with brine (50 mL), dried over anhydrous Na₂SO₄, filtered and concentrated under reduced pressure to afford the product (242 mg, 85%), which was used without further purification. [M+H]⁺=543.6.

### Step 4: (R)-3-(2,6-difluoro-4-((R)-3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazine-1-carbonyl)pyrrolidin-1-yl)phenyl)piperidine-2,6-dione

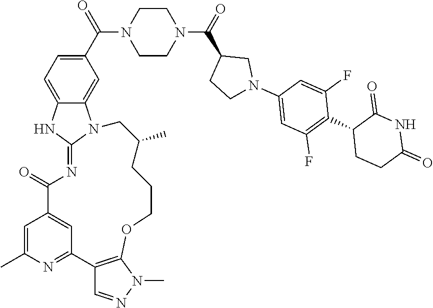 [SMILES: Cc1cc2cc(n1)-c1cnn(C)c1OCCC[C@@H](C)CN1/C(=N/C2=O)Nc2ccc(C(=O)N3CCN(C(=O)[C@@H]4CCN(c5cc(F)c([C@H]6CCC(=O)NC6=O)c(F)c5)C4)CC3)cc21]

To a 40-mL microwave vial equipped with a magnetic stir bar was added (R,E)-1¹,2⁶,7-trimethyl-5⁶-(piperazine-1-carbonyl)-5²,5¹-dihydro-1¹H,5¹H-11-oxa-4-aza-5(2,1)-benzo[d]imidazola-2(2,4)-pyridina-1(4,5)-pyrazolacycloundecaphan-3-one (54.3 mg, 0.10 mmol), (R)-1-(4-((R)-2,6-dioxopiperidin-3-yl)-3,5-difluorophenyl)pyrrolidine-3-carboxylic acid (40.6 mg, 0.12 mmol) and DCM (8 mL). To the reaction mixture was added N,N-diethylpropylethylamine (87 mL, 0.5 mmol) and T3P (50 wt %, 127 mg, 0.2 mmol). After stirring for 1.5 h at room temperature, the reaction mixture was diluted with brine (20 mL) and the layers were separated. The aqueous layer was extracted with DCM (3×15 mL). The combined organic layers were washed with brine (30 mL), dried over anhydrous Na₂SO₄, filtered and concentrated under reduced pressure. Purification by flash column chromatography (0-10% MeOH in DCM) followed by prep-HPLC chromatography (0.1% FA in water:acetonitrile=90:10-50:50 gradient elution) afforded the title compound (62 mg, 72%). ¹H NMR (500 MHz, DMSO) δ 12.88 (s, 1H), 10.84 (s, 1H), 8.43 (s, 1H), 7.93 (s, 1H), 7.72 (s, 1H), 7.60 (d, J=8.0 Hz, 1H), 7.58 (s, 1H), 7.31 (d, J=8.5 Hz, 1H), 6.23 (d, J=12.0 Hz, 2H), 4.41-4.33 (m, 1H), 4.21 (d, J=12.5 Hz, 1H), 4.04-4.00 (m, 3H), 3.73 (s, 3H), 3.67-3.50 (m, 8H), 3.48-3.41 (m, 3H), 3.28-3.21 (m, 3H), 2.83-2.74 (m, 2H), 2.56 (s, 3H), 2.25-2.17 (m, 2H), 2.15-2.04 (m, 2H), 2.02-1.89 (m, 3H), 1.51-1.42 (m, 1H), 0.83 (d, J=6.5 Hz, 3H); [M+H]⁺=863.7.

### Example 97: (R)-3-(2,6-difluoro-4-(2-(4-((R,E)-1¹,2⁶,7-trimethyl-3-oxo-5², 5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazin-1-yl) ethyl)phenyl)piperidine-2,6-dione

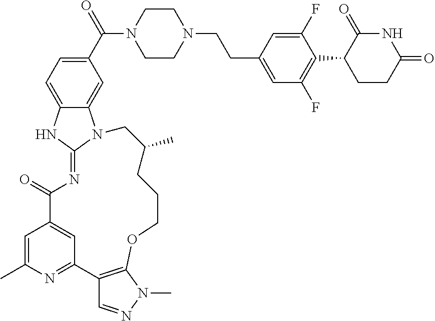 [SMILES: Cc1cc2cc(n1)-c1cnn(C)c1OCCC[C@@H](C)CN1/C(=N/C2=O)Nc2ccc(C(=O)N3CCN(CCc4cc(F)c([C@H]5CCC(=O)NC5=O)c(F)c4)CC3)cc21]

To a 40-mL microwave vial equipped with a magnetic stir bar was added (R,E)-1¹,2⁶,7-trimethyl-5⁶-(piperazine-1-carbonyl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (108.5 mg, 0.20 mmol), (R)-2-(4-(2,6-dioxopiperidin-3-yl)-3,5-difluorophenyl)acetaldehyde (106.8 mg, 0.40 mmol) and DCM (8 mL). To the reaction mixture was added 1 drop of Ti(OiPr)₄. After stirring for 1 h at 40° C., NaBH(OAc)₃ (127 mg, 0.6 mmol) was added. After stirring for another 2 h at 40° C., the reaction mixture was diluted with sat. aq. NaHCO₃(20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by prep-TLC followed by prep-HPLC chromatography (0.1% FA in water:acetonitrile=90:10-50:50 gradient elution) afforded the title compound (128 mg, 81%). ¹H NMR (500 MHz, DMSO) δ 12.86 (s, 1H), 10.95 (s, 1H), 8.42 (s, 1H), 7.93 (s, 1H), 7.68 (s, 1H), 7.62-7.57 (m, 2H), 7.26 (d, J=7.5 Hz, 1H), 7.04 (d, J=10.0 Hz, 2H), 4.40-4.31 (m, 1H), 4.21-4.18 (m, 2H), 4.04-3.99 (m, 2H), 3.73 (s, 3H), 3.64-3.39 (m, 5H), 2.84-2.73 (m, 5H), 2.61-2.59 (m, 3H), 2.56 (s, 3H), 2.49-2.42 (m, 2H), 2.26-2.06 (m, 2H), 2.00-1.91 (m, 3H), 1.52-1.40 (m, 1H), 0.81 (d, J=5.5 Hz, 3H); [M+H]⁺=794.6.

### Example 98: (3R)-3-(2,6-difluoro-4-((3R)-3-(6-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,6-di azabicyclo[3.1.1]heptane-3-carbonyl)pyrrolidin-1-yl)phenyl)piperidine-2,6-dione

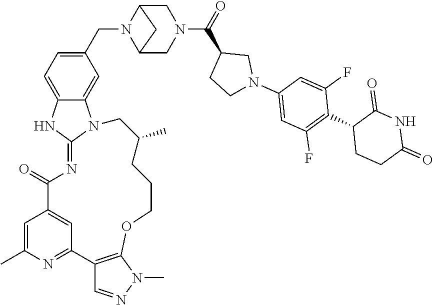 [SMILES: Cc1cc2cc(n1)-c1cnn(C)c1OCCC[C@@H](C)CN1/C(=N/C2=O)Nc2ccc(CN3C4CC3CN(C(=O)[C@@H]3CCN(c5cc(F)c([C@H]6CCC(=O)NC6=O)c(F)c5)C3)C4)cc21]

The titled compound was prepared in a manner similar to that in Example 5.

¹H NMR (500 MHz, DMSO) δ 12.68 (s, 1H), 10.85 (d, J=5.3 Hz, 1H), 8.42 (s, 1H), 7.92 (s, 1H), 7.57 (d, J=9.7 Hz, 2H), 7.48 (d, J=8.0 Hz, 1H), 7.25 (dd, J=14.8, 8.4 Hz, 1H), 6.27 (t, J=13.0 Hz, 2H), 4.34 (dd, J=8.2, 5.6 Hz, 1H), 4.24-4.14 (m, 1H), 4.07-3.87 (m, 4H), 3.73 (s, 3H), 3.68 (s, 3H), 3.62-3.52 (m, 5H), 3.47-3.33 (m, 6H), 2.86-2.73 (m, 2H), 2.55 (s, 3H), 2.33-2.05 (m, 4H), 2.02-1.82 (m, 3H), 1.53-1.40 (m, 2H), 0.81 (t, J=7.0 Hz, 3H); [M+H]⁺=861.7.

### Example 99: (R)-3-(2,6-difluoro-4-((R)-34(S)-3-methyl-4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)methyl)piperazine-1-carbonyl)pyrrolidin-1-yl)phenyl)piperidine-2,6-dione

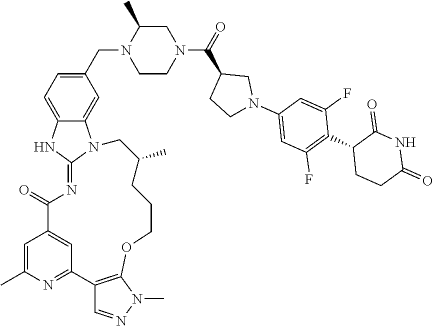 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)C[C@@H]3C)cc21]

The titled compound was prepared in a manner similar to that in Example 5.

¹H NMR (500 MHz, DMSO) δ 12.70 (s, 1H), 10.84 (s, 1H), 8.43 (s, 1H), 7.92 (s, 1H), 7.57 (s, 1H), 7.53 (d, J=6.1 Hz, 1H), 7.49 (d, J=8.0 Hz, 1H), 7.20 (t, J=6.8 Hz, 1H), 6.22 (s, 1H), 6.20 (s, 1H), 4.41-4.33 (m, 1H), 4.21 (d, J=11.7 Hz, 1H), 4.06-3.92 (m, 6H), 3.86-3.66 (m, 5H), 3.51 (dd. J=15.7, 7.5 Hz, 1H), 3.46-3.38 (m, 3H), 3.27-2.92 (m, 4H), 2.85-2.73 (m, 2H), 2.69-2.60 (m, 1H), 2.56 (s, 3H), 2.22-1.90 (m, 8H), 1.54-1.42 (m, 1H), 1.15-1.09 (m, 3H), 0.82 (d, J=6.3 Hz, 3H): [M+H]⁺=863.7.

### Example 100: (R)-3-(2,6-difluoro-4-((R)-3-((R)-3-methyl-4-(((R,E)-1¹,2⁶,7-trimethyl-3-oxo-5²,5³-dihydro-1¹H,5¹H -11-oxa-4-aza-5(2,1)-benzo[d]imidazola-2(2,4)-pyridina-(4,5)-pyrazolacycloundecaphane-5⁶-yl)methyl)piperazine-1-carbonyl)pyrrolidin-1-yl)phenyl)piperidine-2,6-dione

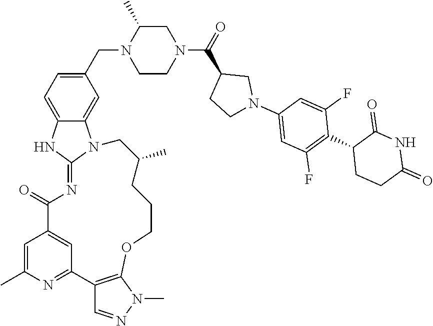 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)C[C@H]3C)cc21]

The titled compound was prepared in a manner similar to that in Example 5.

¹H NMR (500 MHz, DMSO) δ 12.70 (s, 1H), 10.84 (s, 1H), 8.43 (s, 1H), 7.92 (s, 1H), 7.57 (s, 1H), 7.53 (s, 1H), 7.49 (d, J=8.2 Hz, 1H), 7.21 (d, J=8.2 Hz, 1H), 6.22 (s, 1H), 6.20 (s, 1H), 4.42-4.32 (m, 1H), 4.21 (d, J=13.2 Hz, 1H), 4.07-3.91 (m, 4H), 3.87-3.69 (m, 5H), 3.62-3.33 (m, 6H), 3.28-2.89 (m, 4H), 2.84-2.72 (m, 2H), 2.72-2.62 (m, 1H), 2.56 (s, 3H), 2.23-1.92 (m, 8H), 1.52-1.42 (m, 1H), 1.16-1.10 (m, 3H), 0.81 (d, J=5.3 Hz, 3H); [M+H]⁺=863.7.

### Example 101: (3R)-3-(2,6-difluoro-4-(2-(6-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,6-diazabicyclo[3.1.1]heptan-3-yl)ethyl)phenyl)piperidine-2,6-dione

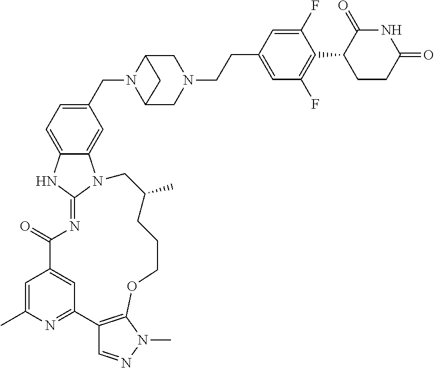 [SMILES: Cc1cc2cc(n1)-c1cnn(C)c1OCCC[C@@H](C)CN1/C(=N/C2=O)Nc2ccc(CN3C4CC3CN(CCc3cc(F)c([C@H]5CCC(=O)NC5=O)c(F)c3)C4)cc21]

The titled compound was prepared in a manner similar to that in Example 14. ¹H NMR (500 MHz, DMSO) δ 12.68 (s, 1H), 10.95 (s, 1H), 8.43 (s, 1H), 7.92 (s, 1H), 7.56 (s, 1H), 7.53 (s, 1H), 7.46 (d, J=7.9 Hz, 1H), 7.22 (d, J=7.2 Hz, 1H), 7.09 (s, 1H), 7.07 (s, 1H), 4.36 (s, 1H), 4.20 (d, J=13.3 Hz, 2H), 4.00 (s, 1H), 3.98-3.89 (m, 1H), 3.73 (s, 3H), 3.67 (s, 2H), 3.48 (s, 2H), 3.25-3.19 (m, 1H), 3.02 (s, 2H), 2.95-2.77 (m, 8H), 2.55 (s, 3H), 2.34-2.08 (m, 3H), 2.01-1.93 (m, 3H), 1.80 (s, 1H), 1.47 (s, 1H), 0.81 (d, J=6.5 Hz, 3H); [M+H]⁺=792.7.

### Example 102: (3R)-3-(2,6-difluoro-4-(2-(3-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,6-diazabicyclo[3.1.1]heptan-6-yl)ethyl)phenyl)piperidine-2,6-dione

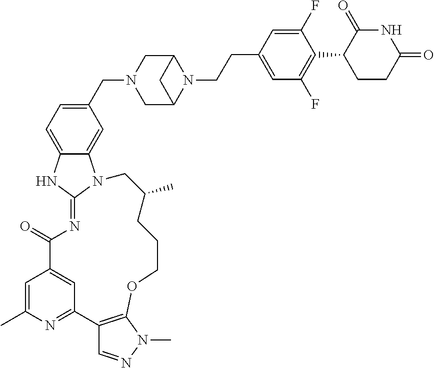 [SMILES: Cc1cc2cc(n1)-c1cnn(C)c1OCCC[C@@H](C)CN1/C(=N/C2=O)Nc2ccc(CN3CC4CC(C3)N4CCc3cc(F)c([C@H]4CCC(=O)NC4=O)c(F)c3)cc21]

The titled compound was prepared in a manner similar to that in Example 14.

¹H NMR (500 MHz, DMSO) δ 12.67 (s, 1H), 10.93 (s, 1H), 8.42 (s, 1H), 7.92 (s, 1H), 7.57 (s, 1H), 7.54 (s, 1H), 7.48 (d, J=8.1 Hz, 1H), 7.20 (d, J=8.0 Hz, 1H), 6.91 (s, 1H), 6.89 (s, 1H), 4.38-4.30 (m, 1H), 4.21-4.13 (m, 2H), 4.02-3.81 (m, 4H), 3.73 (s, 3H), 3.49 (s, 2H), 3.28-3.15 (m, 2H), 3.00 (dd, J=10.8, 5.3 Hz, 2H), 2.81-2.67 (m, 4H), 2.56 (s, 3H), 2.47-2.35 (m, 3H), 2.22 (s, 2H), 2.11-2.06 (m, 1H), 2.01-1.84 (m, 4H), 1.44 (td, J=11.5, 4.2 Hz, 1H), 0.76 (d, J=6.4 Hz, 3H): [M+H]⁺=792.7.

### Example 103: (3R)-3-(2,6-difluoro-4-(2-(5-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,5-diazabicyclo[2.2.2]octan-2-yl)ethyl)phenyl)piperidine-2,6-dione

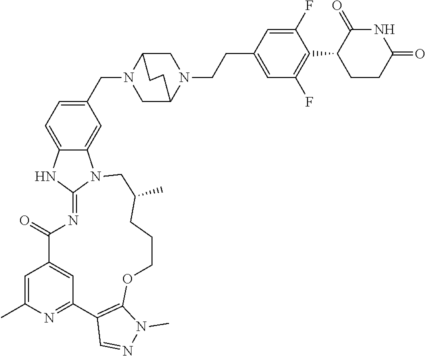 [SMILES: Cc1cc2cc(n1)-c1cnn(C)c1OCCC[C@@H](C)CN1/C(=N/C2=O)Nc2ccc(CN3CC4CCC3CN4CCc3cc(F)c([C@H]4CCC(=O)NC4=O)c(F)c3)cc21]

The titled compound was prepared in a manner similar to that in Example 14.

¹H NMR (500 MHz, DMSO) δ 12.69 (s, 1H), 10.95 (s, 1H), 8.42 (s, 1H), 7.92 (s, 1H), 7.56 (s, 1H), 7.53 (s, 1H), 7.47 (d, J=8.0 Hz, 1H), 7.21 (dd, J=7.8, 4.3 Hz, 1H), 7.06 (s, 1H), 7.04 (s, 1H), 4.39-4.32 (m, 1H), 4.20 (d, J=12.5 Hz, 2H), 4.00 (s, 1H), 3.96-3.87 (m, 1H), 3.80 (d, J=17.8 Hz, 2H), 3.73 (s, 3H), 3.25-3.20 (m, 1H), 3.05 (d, J=8.9 Hz, 1H), 2.92 (d, J=9.0 Hz, 1H), 2.86-2.60 (m, 10H), 2.56 (s, 3H), 2.27-2.07 (m, 2H), 2.04-1.80 (m, 5H), 1.65-1.40 (m, 3H), 0.80 (t, J=5.5 Hz, 3H); [M+H]⁺=806.7.

### Example 104: (R)-3-(2,6-difluoro-4-(2-((S)-3-methyl-4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl) piperazin-1-yl)ethyl)phenyl)piperidine-2,6-dione

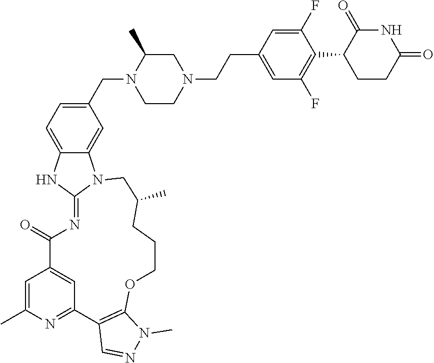 [SMILES: Cc1cc2cc(n1)-c1cnn(C)c1OCCC[C@@H](C)CN1/C(=N/C2=O)Nc2ccc(CN3CCN(CCc4cc(F)c([C@H]5CCC(=O)NC5=O)c(F)c4)C[C@@H]3C)cc21]

The titled compound was prepared in a manner similar to that in Example 14.

¹H NMR (500 MHz, DMSO) δ 12.70 (s, 1H), 10.94 (s, 1H), 8.43 (s, 1H), 7.93 (d, J=9.7 Hz, 1H), 7.58 (d, J=9.4 Hz, 1H), 7.49 (s, 1H), 7.47 (s, 1H), 7.17 (d, J=8.1 Hz, 1H), 7.02 (s, 1H), 7.00 (s, 1H), 4.36 (d, J=4.9 Hz, 1H), 4.19 (dd, J=13.7, 8.8 Hz, 2H), 4.02 (dd, J=16.1, 9.6 Hz, 2H), 3.96-3.88 (m, 1H), 3.73 (s, 3H), 3.27 (s, 2H), 2.86-2.69 (m, 5H), 2.65-2.52 (m, 8H), 2.28-2.06 (m, 4H), 2.05-1.84 (m, 4H), 1.53-1.42 (m, 1H), 1.11 (d, J=6.0 Hz, 3H), 0.81 (d, J=6.4 Hz, 3H); [M+H]*=794.7.

### Example 105: (R)-3-(2,6-difluoro-4-(2-((R)-3-methyl-4-(((R,E)-1¹,2,7-trimethyl-3-oxo-5²,5′-dihydro-1¹H,5¹H-11-oxa-4-aza-5(2,1)-benzo[d]imidazola-2(2,4)-pyridina-(4,5)-pyrazolacycloundecaphane-5⁶-yl)methyl) piperazin-1-yl)ethyl)phenyl)piperidine-2,6-dione

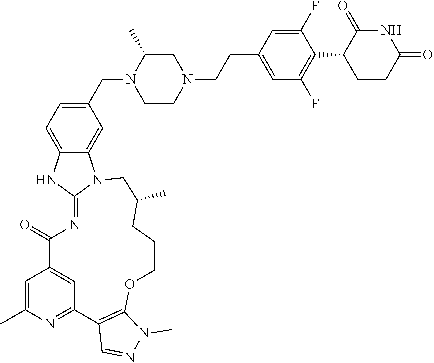 [SMILES: Cc1cc2cc(n1)-c1cnn(C)c1OCCC[C@@H](C)CN1/C(=N/C2=O)Nc2ccc(CN3CCN(CCc4cc(F)c([C@H]5CCC(=O)NC5=O)c(F)c4)C[C@H]3C)cc21]

The titled compound was prepared in a manner similar to that in Example 14.

¹H NMR (500 MHz, DMSO) δ 12.69 (s, 1H), 10.94 (s, 1H), 8.43 (s, 1H), 7.92 (s, 1H), 7.57 (s, 1H), 7.50 (s, 1H), 7.48 (d, 0.1=8.3 Hz, 1H), 7.18 (d, J=8.1 Hz, 1H), 7.02 (s, 1H), 7.00 (s, 1H), 4.40-4.31 (m, 1H), 4.25-4.15 (m, 2H), 4.08 (d, J=12.4 Hz, 1H), 4.00 (s, 1H), 3.96-3.89 (m, 1H), 3.73 (s, 3H), 3.31-3.14 (m, 3H), 2.84-2.64 (m, 5H), 2.68-2.52 (m, 7H), 2.26-1.88 (m, 8H), 1.51-1.42 (m, 1H), 1.11 (d, J=5.7 Hz, 3H), 0.80 (d, J=6.2 Hz, 3H); [M+H]⁺=794.7.

### Example 106: (R)-3-(2,6-difluoro-4-((R)-3-((R)-2-methyl-4-(((R,E)-1¹,2⁶m7-trimethyl-3-oxo-5²,5³-dihydro-1¹H,5¹H -11-oxa-4-aza-5(2,1)-benzo[d]imidazola-2(2,4)-pyridina-1(4,5)-pyrazolacycloundecaphane-5⁶-yl)methyl)piperazine-1-carbonyl)pyrrolidin-1-yl)phenyl)piperidine-2,6-dione

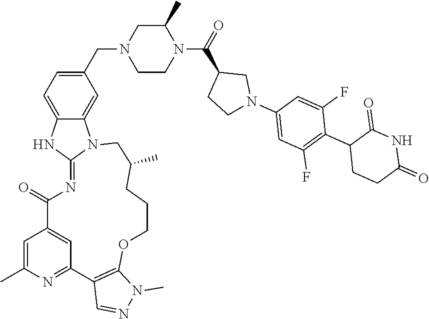 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c(C6CCC(=O)NC6=O)c(F)c5)C4)[C@H](C)C3)cc21]

The titled compound was prepared in a manner similar to that in Example 5.

¹H NMR (500 MHz, DMSO) δ 12.65 (s, 1H), 10.77 (s, 1H), 8.36 (s, 1H), 7.86 (s, 1H), 7.49 (d, J=6.8 Hz, 2H), 7.43 (d, 1H), 7.13 (s, 1H), 6.15 (d, J=7.2 Hz, 2H), 4.28 (s, 1H), 4.14 (d, J=6.1 Hz, 3H), 3.93 (s, 3H), 3.86 (s, 2H), 3.78 (d, J=6.3 Hz, 2H), 3.66 (s, 3H), 2.82 (s, 2H), 2.70 (m, 3H), 2.58-2.51 (m, 2H), 2.49 (m, 3H), 2.14 (s, 3H), 1.93 (m, 8H), 1.40 (s, 1H), 1.24 (d, J=5.5 Hz, 1H), 1.08 (d, J=6.4 Hz, 2H), 0.75 (d, J=6.1 Hz, 3H). [M+H]⁺=863.4

### Example 107: (R)-3-(2,6-difluoro-4-(2-((R)-2-methyl-4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl) piperazin-1-yl)ethyl)phenyl)piperidine-2,6-dione

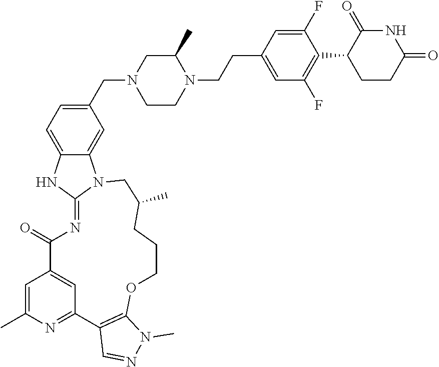 [SMILES: Cc1cc2cc(n1)-c1cnn(C)c1OCCC[C@@H](C)CN1/C(=N/C2=O)Nc2ccc(CN3CCN(CCc4cc(F)c([C@H]5CCC(=O)NC5=O)c(F)c4)[C@H](C)C3)cc21]

The titled compound was prepared in a manner similar to that in Example 14.

¹H NMR (500 MHz, DMSO) δ 12.70 (s, 1H), 10.94 (s, 1H), 8.42 (s, 1H), 7.92 (s, 1H), 7.57 (s, 1H), 7.48 (d, J=7.1 Hz, 2H), 7.17 (d, J=6.1 Hz, 1H), 7.01 (d, J=6.8 Hz, 2H), 4.36 (s, 1H), 4.19 (d, J=6.4 Hz, 2H), 4.00 (s, 1H), 3.96-3.89 (m, 1H), 3.73 (s, 3H), 3.53 (m, 4H), 2.85-2.77 (m, 4H), 2.71 (d, J=7.0 Hz, 2H), 2.61 (m, 2H), 2.56 (s, 3H), 2.37 (d, J=6.7 Hz, 1H), 2.27-2.06 (m, 4H), 1.98 (d, J=6.2 Hz, 2H), 1.91 (s, 2H), 1.47 (s, 1H), 0.95 (d, J=6.9 Hz, 3H), 0.81 (d, J=6.3 Hz, 3H). [M+H]⁺=794.4

### Example 108: (R)-3-(2,6-difluoro-4-((R)-3-(8-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-2-oxa-5,8-diazaspiro[3.5]nonane-5-carbonyl)pyrrolidin-1-yl)phenyl)piperidine-2,6-dione

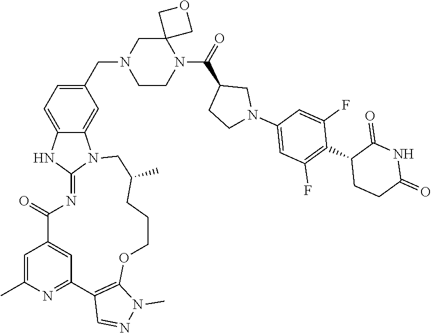 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)C4(COC4)C3)cc21]

The titled compound was prepared in a manner similar to that in Example 5.

¹H NMR (500 MHz, DMSO) δ 12.83-12.49 (m, 1H), 10.84 (s, 1H), 8.43 (s, 1H), 8.32 (s, 1H), 7.92 (s, 1H), 7.74 (d, J=7.9 Hz, 1H), 7.55 (d, J=6.9 Hz, 1H), 7.29 (d, J=6.2 Hz, 1H), 6.22 (d, J=12.7 Hz, 2H), 4.60 (s, 2H), 4.36 (s, 1H), 4.23 (d, J=8.9 Hz, 3H), 4.00 (s, 3H), 3.73 (s, 5H), 3.67-3.53 (m, 4H), 2.74 (m, 4H), 2.55 (d, J=6.1 Hz, 5H), 2.36 (s, 4H), 2.16 (s, 2H), 2.08 (d, J=6.8 Hz, 3H), 1.95 (s, 2H), 1.46 (s, 1H), 0.86-0.64 (m, 3H). [M+H]⁺=891.4.

### Example 59: (R)-3-(2,6-difluoro-4-((R)-3-(4-(((R,E)-5⁷-fluoro-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl) piperazine-1-carbonyl)pyrrolidin-1-yl)phenyl)piperidine-2,6-dione

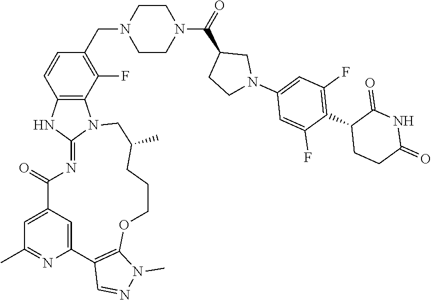 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3)c(F)c21]

The titled compound was prepared in a manner similar to that in Example 5. ¹H NMR (500 MHz, DMSO) δ 12.94 (s, 1H), 10.84 (s, 1H), 8.40 (s, 1H), 7.92 (s, 1H), 7.57 (s, 1H), 7.38 (d, J=8.0 Hz, 1H), 7.26 (s, 1H), 6.21 (d, J=12.0 Hz, 2H), 4.39-4.28 (m, 2H), 4.07-3.90 (m, 3H), 3.73 (s, 3H), 3.63-3.17 (m, 1H), 2.85-2.72 (m, 2H), 2.56 (s, 3H), 2.31-1.81 (m, 8H), 1.69-1.37 (m, 3H), 1.02-0.80 (m, 5H); [M+H]⁺=867.8.

### Example 64: (R)-3-(4-(3-(4-(((R,E)-13-(difluoromethyl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carbonyl)azetidin-1-yl)-2,6-difluorophenyl)piperidine-2,6-dione

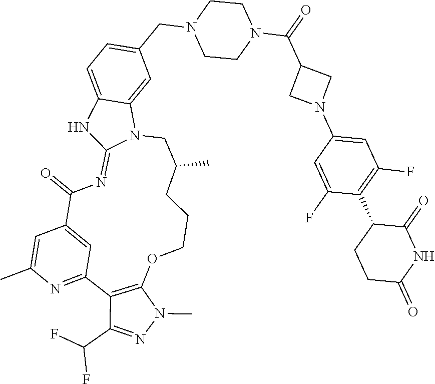 [SMILES: Cc1cc2cc(n1)-c1c(C(F)F)nn(C)c1OCCC[C@@H](C)CN1/C(=N/C2=O)Nc2ccc(CN3CCN(C(=O)C4CN(c5cc(F)c([C@H]6CCC(=O)NC6=O)c(F)c5)C4)CC3)cc21]

The titled compound was prepared in a manner similar to that in Example 5. ¹H NMR (50(0 MHz, DMSO) δ 12.74 (s, 1H), 10.85 (s, 1H), 8.51 (s, 1H), 7.80 (t, J=54.5 Hz, 1H), 7.63 (s, 1H), 7.53 (s, 1H), 7.50 (d, J=8.0 Hz. IH), 7.20 (d, J=8.0 Hz, 1H), 6.15 (d, J=11.5 Hz, 2H), 4.45-4.36 (m, 1H), 4.21 (d, J=12.6 Hz, 1H), 4.10-3.79 (m, 13H), 3.61 (s, 2H), 3.50 (s, 2H), 2.90-2.72 (m, 3H), 2.58 (s, 3H), 2.43-2.33 (m, 3H), 2.28-2.18 (m, 1H), 2.14-1.88 (m, 5H), 1.52-1.42 (m, 1H), 0.82 (d, J=6.0 Hz, 3H). [M+H]⁺=885.70.

### Example 144: (R,E)-3-(4-(2-(4-((1¹,2⁶-dimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)ethyl)-2,6-difluorophenyl)piperidine-2,6-dione

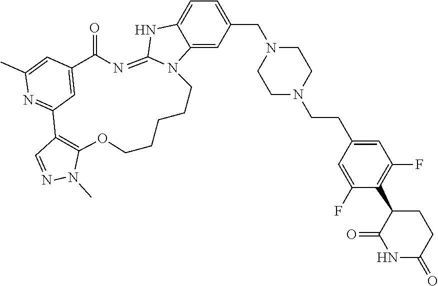 [SMILES: Cc1cc2cc(n1)-c1cnn(C)c1OCCCCCN1/C(=N/C2=O)Nc2ccc(CN3CCN(CCc4cc(F)c([C@H]5CCC(=O)NC5=O)c(F)c4)CC3)cc21]

The titled compound was prepared in a manner similar to that in Example 14. ¹H NMR (500 MHz, DMSO) δ 12.65 (s, 1H), 10.94 (s, 1H), 8.41 (s, 1H), 7.92 (s, 1H), 7.57 (s, 1H), 7.52-7.45 (m, 2H), 7.18 (d, J=8.1 Hz, 1H), 7.01 (d, J=10.2 Hz, 2H), 4.21 (s, 5H), 3.73 (s, 3H), 3.55 (s, 2H), 2.77 (dt, J=14.4, 10.1 Hz, 3H), 2.55 (d, J=12.4 Hz, 7H), 2.36 (s, 7H), 2.07 (m, 6H), 1.80 (s, 2H). [M+H]=766.7.

### Example 147: (R)-3-(2,6-difluoro-4-(3-((4-(((R,E)-1¹,2⁶,5,7-tetramethyl-3-oxo-5²,5′-dihydro-1¹H,5¹H-11-oxa-4-aza-5(2,1)-benzo[d]imidazola-2(2,4)-pyridina-(4,5)-pyrazolacycloundecaphane-5⁶-yl)methyl)piperazin-1-yl)methyl)azetidin-1-yl)phenyl)piperidine-2,6-dione

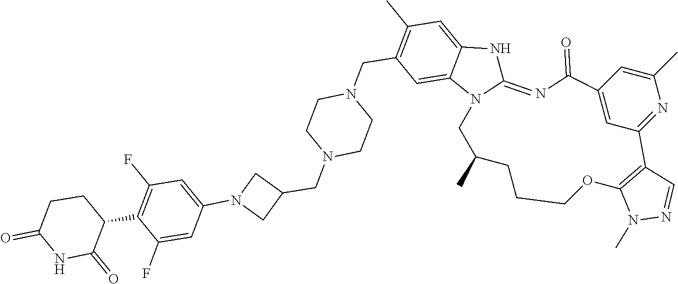 [SMILES: Cc1cc2cc(n1)-c1cnn(C)c1OCCC[C@@H](C)CN1/C(=N/C2=O)Nc2cc(C)c(CN3CCN(CC4CN(c5cc(F)c([C@H]6CCC(=O)NC6=O)c(F)c5)C4)CC3)cc21]

The titled compound was prepared in a manner similar to that in Example 14. ¹H NMR (500 MHz, DMSO) δ 12.61 (s, 1H), 10.84 (s, 1H), 8.42 (s, 1H), 7.92 (s, 1H), 7.56 (s, 1H), 7.43 (s, 1H), 7.33 (s, 1H), 6.09 (d, J=11.3 Hz, 2H), 4.35 (s, 1H), 4.20 (d, J=12.3 Hz, 1H), 4.04-3.98 (m, 2H), 3.91 (t, J=6.9 Hz, 3H), 3.73 (s, 3H), 3.53 (d, J=13.1 Hz, 1H), 3.48-3.46 (m, 3H), 2.90 (s, 1H), 2.76 (d, J=11.8 Hz, 2H), 2.55 (s, 4H), 2.54 (s, 1H), 2.49-2.45 (m, 4H), 2.39 (s, 6H), 2.37-2.34 (m, 2H), 2.21 (s, 1H), 2.06 (d, J=10.8 Hz, 1H), 2.00-1.88 (m, 3H), 1.45 (s, 1H), 0.80 (d, J=6.3 Hz, 3H); [M+H]⁺=835.7.

### Example 148: (R)-3-(2,6-difluoro-4-(3-((4-(((R,E)-1¹,2⁶,5⁷,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazin-1-yl)methyl)azetidin-1-yl)phenyl)piperidine-2,6-dione

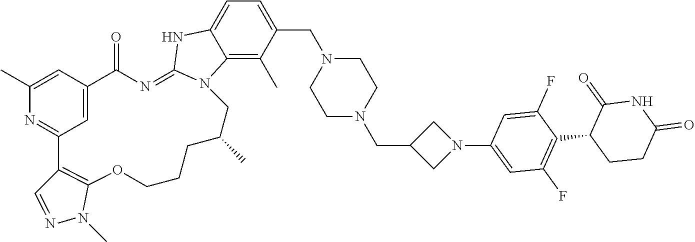 [SMILES: Cc1cc2cc(n1)-c1cnn(C)c1OCCC[C@@H](C)CN1/C(=N/C2=O)Nc2ccc(CN3CCN(CC4CN(c5cc(F)c([C@H]6CCC(=O)NC6=O)c(F)c5)C4)CC3)c(C)c21]

The titled compound was prepared in a manner similar to that in Example 14. ¹H NMR (500 MHz, DMSO) δ 12.77 (s, 1H), 10.85 (s, 1H), 8.38 (s, 1H), 7.93 (s, 1H), 7.57 (s, 1H), 7.38 (d, J=7.7 Hz, 1H), 7.11 (d, J=7.4 Hz, 1H), 6.09 (d, J=11.3 Hz, 2H), 4.36-4.27 (m, 3H), 4.07-3.96 (m, 2H), 3.92 (s, 2H), 3.73 (s, 3H), 3.64-3.53 (m, 1H), 3.47 (s, 3H), 3.30-3.24 (m, 2H), 2.90 (s, 2H), 2.83-2.71 (m, 2H), 2.67 (s, 3H), 2.55-2.51 (m, 6H), 2.36 (s, 5H), 2.22 (s, 1H), 2.11-2.03 (m, 2H), 1.98-1.90 (m, 1H), 1.84 (s, 1H), 1.51 (s, 1H), 0.89 (d, J=6.5 Hz, 3H). [M+H]⁺=835.6

### Example 150: (R)-3-(2,6-difluoro-4-(3-((4-(((R,E)-1¹,2⁶,7-trimethyl-3-oxo-5²,5¹-dihydro-1¹H,5¹H-11-oxa-4-aza-5(2,1)-benzo[d]imidazola-2(2,4)-pyridina-(4,5)-pyrazolacycloundecaphane-5⁶-yl)methyl)piperazin-1-yl)methyl)azetidin-1-yl)phenyl)piperidine-2,6-dione

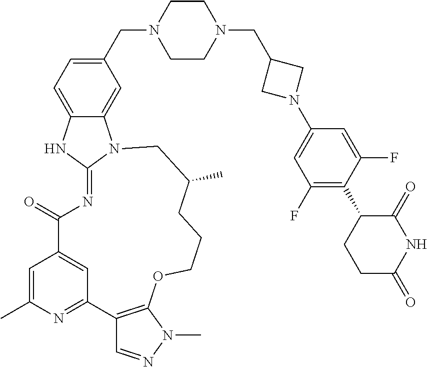 [SMILES: Cc1cc2cc(n1)-c1cnn(C)c1OCCC[C@@H](C)CN1/C(=N/C2=O)Nc2ccc(CN3CCN(CC4CN(c5cc(F)c([C@H]6CCC(=O)NC6=O)c(F)c5)C4)CC3)cc21]

The titled compound was prepared in a manner similar to that in Example 14. ¹H NMR (500 MHz, DMSO) δ 12.63 (s, 1H), 10.78 (s, 1H), 8.36 (s, 1H), 7.85 (s, 1H), 7.50 (s, 1H), 7.42 (s, 1H), 7.41 (d, J=8.5 Hz, 1H), 7.10 (d, J=8.0 Hz, 1H), 6.02 (d, J=12.0 Hz, 2H), 4.33-4.25 (m, 1H), 4.16-4.09 (m, 1H), 3.94 (dd, J=12.0.4.0 Hz, 2H), 3.88-3.82 (m, 3H), 3.66 (s, 3H), 3.49 (s, 2H), 3.38-3.35 (m, 4H), 2.86-2.79 (m, 1H), 2.74-2.67 (m, 2H), 2.44-2.39 (m, 7H), 2.38-2.24 (m, 5H), 2.20-2.10 (m, 1H), 2.03-1.81 (m, 4H), 1.45-1.34 (m, 1H), 0.74 (d, J=5.5 Hz, 3H), [M+H]⁺=821.8.

### Example 154: (R)-3-(2,6-difluoro-4-(3-(4-(((S,E)-11,26,54,7-tetramethyl-3-oxo-5²,5¹-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)piperazine-1-carbonyl)azetidin-1-yl)phenyl)piperidine-2,6-dione

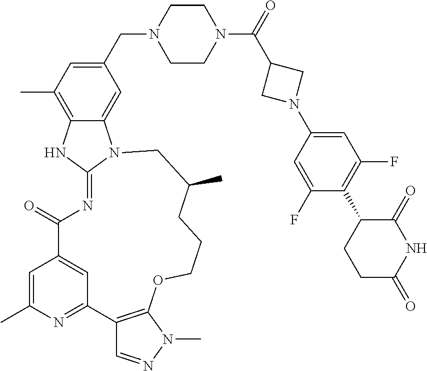 [SMILES: Cc1cc2cc(n1)-c1cnn(C)c1OCCC[C@H](C)CN1/C(=N/C2=O)Nc2c(C)cc(CN3CCN(C(=O)C4CN(c5cc(F)c([C@H]6CCC(=O)NC6=O)c(F)c5)C4)CC3)cc21]

The titled compound was prepared in a manner similar to that in Example 5. ¹H NMR (500 MHz, DMSO) δ 12.17 (s, 1H), 10.86 (s, 1H), 8.42 (s, 1H), 7.92 (s, 1H), 7.55 (s, 1H), 7.37 (s, 1H), 7.03 (s, 1H), 6.15 (d, J=11.2 Hz, 2H), 4.35 (d, J=4.5 Hz, 1H), 4.20 (d, J=12.5 Hz, 1H), 4.03 (d, J=9.8 Hz, 4H), 3.96-3.86 (m, 3H), 3.82 (d, J=6.5 Hz, 1H), 3.73 (s, 3H), 3.58 (s, 2H), 3.50 (s, 2H), 3.32 (s, 3H), 2.85-2.71 (m, 2H), 2.54 (d, J=12.4 Hz, 6H), 2.37 (s, 4H), 2.21 (s, 1H), 1.98-1.96 (m, 4H), 1.46 (s, 1H), 0.82 (d, J=6.3 Hz, 3H): [M+H]⁺=849.7.

### Example 156: (R)-3-(2,6-difluoro-4-(3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)oxy)piperidine-1-carbonyl)azetidin-1-yl)phenyl)piperidine-2,6-dione

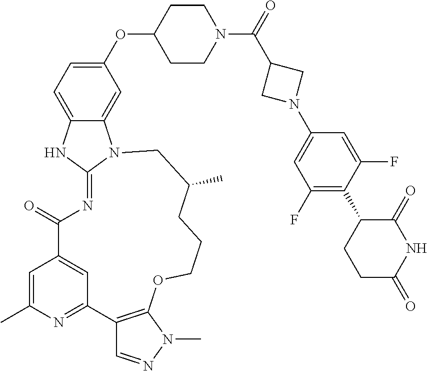 [SMILES: Cc1cc2cc(n1)-c1cnn(C)c1OCCC[C@@H](C)CN1/C(=N/C2=O)Nc2ccc(OC3CCN(C(=O)C4CN(c5cc(F)c([C@H]6CCC(=O)NC6=O)c(F)c5)C4)CC3)cc21]

The titled compound was prepared in a manner similar to that in Example 5. ¹H NMR (500 MHz, DMSO) δ 12.62 (s, 1H), 10.86 (s, 1H), 8.43 (s, 1H), 7.93 (s, 1H), 7.56 (s, 1H), 7.43 (d, J=8.5 Hz, 1H), 7.33 (s, 1H), 6.89 (d, J=8.1 Hz, 1H), 6.17 (d, J=11.2 Hz, 2H), 4.68 (s, 1H), 4.37 (s, 1H), 4.17 (d, J=13.1 Hz, 1H), 4.08-3.85 (m, 10H), 3.73 (s, 3H), 3.55 (s, 1H), 2.87-2.72 (m, 2H), 2.56 (s, 4H), 2.22 (s, 1H), 2.10-1.90 (m, 7H), 1.68-1.55 (m, 2H), 1.45 (s, 1H), 0.82 (d, J=6.1 Hz, 3H). [M+H]⁺=836.7.

### Example 159: (R)-3-(2,6-difluoro-4-(3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidine-1-carbonyl)azetidin-1-yl)phenyl)piperidine-2,6-dione

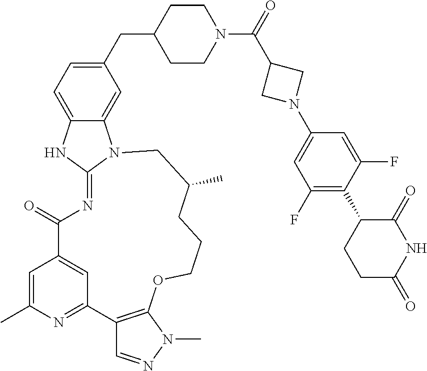 [SMILES: Cc1cc2cc(n1)-c1cnn(C)c1OCCC[C@@H](C)CN1/C(=N/C2=O)Nc2ccc(CC3CCN(C(=O)C4CN(c5cc(F)c([C@H]6CCC(=O)NC6=O)c(F)c5)C4)CC3)cc21]

The titled compound was prepared in a manner similar to that in Example 5. ¹H NMR (500 MHz, DMSO) 512.71 (s, 1H), 10.86 (s, 1H), 8.50 (s, 1H), 8.02 (s, 1H), 7.66 (s, 1H), 7.50-7.39 (m, 2H), 7.06 (d, J=8.1 Hz, 1H), 6.16 (d, J=11.2 Hz, 2H), 4.36 (d, J=8.9 Hz, 2H), 4.20 (d, J=12.8 Hz, 1H), 4.07-3.85 (m, 7H), 3.80 (s, 1H), 3.75 (s, 3H), 3.59 (d, J=11.9 Hz, 1H), 2.93 (t, J=12.6 Hz, 1H), 2.77 (t, J=12.4 Hz, 2H), 2.68-2.53 (m, 6H), 2.22 (s, 1H), 2.13-1.76 (m, 6H), 1.60 (d, J=13.8 Hz, 2H), 1.46 (s, 1H), 1.16-1.00 (m, 2H), 0.82 (d, J=5.4 Hz, 3H). [M+H]⁺=834.7.

### Example 158: (R)-3-(2,6-difluoro-4-(3-(4-((R,E)-1¹,2⁶,7-trimethyl-3-oxo-5²,5-dihydro-1¹H,5¹H-11-oxa-4-aza-5(2, 1)-benzo[d]imidazola-2(2,4)-pyridina-1(4,5)-pyrazolacycloundecaphane-5⁶-yl)piperidine-1-carbonyl)azetidin-1-yl)phenyl)piperidine-2,6-dione

### Step 1: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-3,6-dihydropyridine-1(2H)-carboxylate

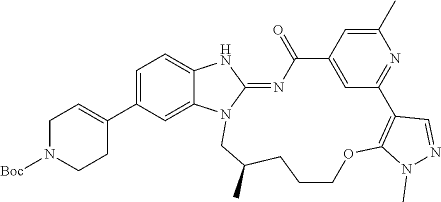 [SMILES: Cc1cc2cc(n1)-c1cnn(C)c1OCCC[C@@H](C)CN1/C(=N/C2=O)Nc2ccc(C3=CCN(C(=O)OC(C)(C)C)CC3)cc21]

To a suspension of tert-butyl 4-(4,4,5,5-tetramethyl-1,3,2-dioxaborolan-2-yl)-3,6-dihydropyridine-1(2H)-carboxylate (455.25 mg, 1.47 mmol) and (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (500 mg, 0.981 mmol) in dioxane (10 mL) and water (2 mL) was added K₂CO₃(406.7 mg, 2.943 mmol) and Pd(dppf)Cl₂(142.37 mg, 0.196 mmol) under N2. The mixture was warmed to 100° C. and stirred for 16 hrs. Then the mixture was cooled to rt and filtered. The filtrate was concentrated in vacuo. The residue was purified by Combi-Flash (silica column, 40 g, DCM:MeOH=15:1) to give product (270.2 mg, 45%). [M+H]⁺=612.3.

### Step 2: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carboxylate

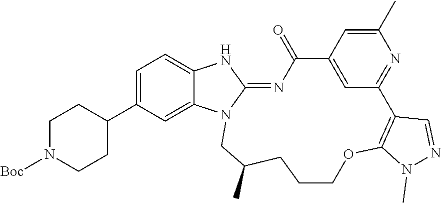 [SMILES: Cc1cc2cc(n1)-c1cnn(C)c1OCCC[C@@H](C)CN1/C(=N/C2=O)Nc2ccc(C3CCN(C(=O)OC(C)(C)C)CC3)cc21]

A mixture of tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-3,6-dihydropyridine-1(2H)-carboxylate (270 mg, 0.441 mmol) and PtO₂(10 wt %, 27 mg) in THF (10 mL) was stirred for 5 h at 40° C. under hydrogen atmosphere. The mixture was allowed to cool down to RT. The resulting mixture was filtered, and the solid was washed with THF. The filtrate was concentrated under reduced pressure to afford product (216.71 mg, 80%), which was used without further purification. [M+H]⁺=614.6.

### Step 3: (R,E)-1¹,2⁶,7-trimethyl-5⁶-(piperidin-4-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one hydrochloride

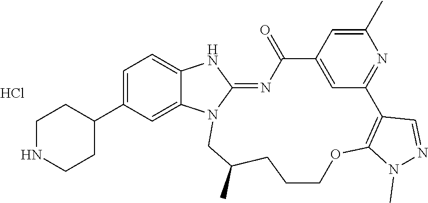 [SMILES: Cl ; Cc1cc2cc(n1)-c1cnn(C)c1OCCC[C@@H](C)CN1/C(=N/C2=O)Nc2ccc(C3CCNCC3)cc21]

To a stirred solution of tert-butyl (R,E)-4-(1¹,2⁶,7-trimethyl-3-oxo-5²,5³-dihydro-1¹,5¹H-11-oxa-4-aza-5(2,1)-benzo[d]imidazola-2(2,4)-pyridina-1(4,5)-pyrazolacycloundecaphane-5⁶-yl)piperidine-1-carboxylate (200 mg, 0.326 mmol) in DCM (6 mL) was added 4M HCl (in dioxane) (2 mL). The reaction mixture was stirred at rt for 2 h and concentrated in vacuum to afford the product (170.29 mg, 95%) [M+H]⁺=514.6.

### Step 4: (R)-3-(2,6-difluoro-4-(3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carbonyl)azetidin-1-yl)phenyl)piperidine-2,6-dione

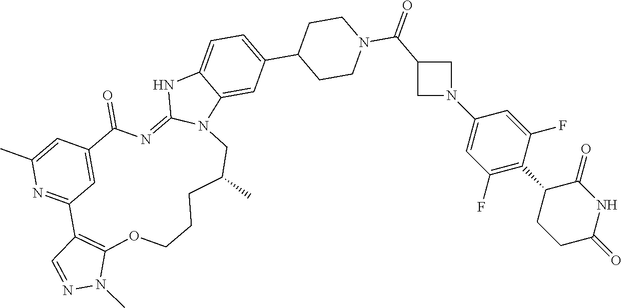 [SMILES: Cc1cc2cc(n1)-c1cnn(C)c1OCCC[C@@H](C)CN1/C(=N/C2=O)Nc2ccc(C3CCN(C(=O)C4CN(c5cc(F)c([C@H]6CCC(=O)NC6=O)c(F)c5)C4)CC3)cc21]

To a solution of (R,E)-1¹,2⁶,7-trimethyl-5⁶-(piperidin-4-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one hydrochloride (50 mg, 0.0908 mmol), (R)-1-(4-(2,6-dioxopiperidin-3-yl)-3,5-difluorophenyl)azetidine-3-carboxylic acid (33.37 mg, 0.109 mmol) and DIEA (35.24 mg, 0.272 mmol) in DCM (3 mL) was added T₃P (72.30 mg, 0.227 mmol) dropwise at room temperature. The resulting mixture was stirred at room temperature for 1 hour. The reaction was quenched with water (20 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC (C-18 column chromatography (0.1% FA in water:acetonitrile=90:10-60:40 gradient elution) to give the desired product (37.6 mg, 50.5%).

¹H NMR (500 MHz, DMSO) δ 12.65 (s, 1H), 10.85 (s, 1H), 8.45 (s, 1H), 7.94 (s, 1H), 7.65-7.53 (m, 2H), 7.45 (d, J=7.7 Hz, 1H), 7.14 (d, J=8.0 Hz, 1H), 6.18 (dd, J=11.0, 6.4 Hz, 2H), 4.57 (s, 1H), 4.36 (s, 1H), 4.18 (d, J=12.3 Hz, 1H), 4.06 (s, 3H), 4.01 (d, J=5.8 Hz, 3H), 3.97 (s, 1H), 3.88 (s, 1H), 3.74 (s, 4H), 3.14 (s, 1H), 2.90 (s, 2H), 2.80 (d, J=13.2 Hz, 2H), 2.74-2.66 (m, 2H), 2.57 (s, 3H), 2.22 (s, 1H), 2.08 (d, J=12.8 Hz, 1H), 1.97 (s, 2H), 1.84 (s, 2H), 1.64 (d, J=11.7 Hz, 2H), 1.46 (s, 1H), 0.82 (s, 3H). [M+H]⁺=820.5.

### Example 162: (R)-3-(2,6-difluoro-4-(4-(4-(((R,E)-1¹,2⁶,7-trimethyl-3-oxo-5², 5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carbonyl)piperidin-1-yl)phenyl)piperidine-2,6-dione

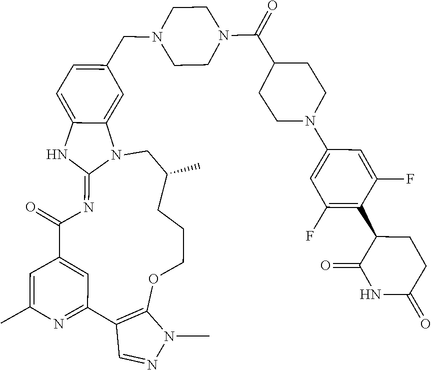 [SMILES: Cc1cc2cc(n1)-c1cnn(C)c1OCCC[C@@H](C)CN1/C(=N/C2=O)Nc2ccc(CN3CCN(C(=O)C4CCN(c5cc(F)c([C@H]6CCC(=O)NC6=O)c(F)c5)CC4)CC3)cc21]

The titled compound was prepared in a manner similar to that in Example 5. ¹H NMR (500 MHz, DMSO) δ 12.72 (s, 1H), 10.86 (s, 1H), 8.43 (s, 1H), 7.92 (s, 1H), 7.60-7.45 (m, 3H), 7.19 (d, J=8.3 Hz, 1H), 6.62 (d, J=12.9 Hz, 2H), 4.41-4.32 (m, 1H), 4.25-4.16 (m, 1H), 4.08-3.90 (m, 3H), 3.81-3.70 (m, 5H), 3.64-3.44 (m, 6H), 2.88-2.73 (m, 4H), 2.60-2.53 (m, 6H), 2.44-2.30 (m, 3H), 2.27-2.17 (m, 1H), 2.12-2.05 (m, 1H), 2.04-1.88 (m, 3H), 1.68-1.54 (m, 3H), 1.53-1.42 (m, 1H), 0.88-0.77 (m, 4H); [M+H]⁺=863.5.

### Example 163: (3R)-3-(2,6-difluoro-4-((3R)-3-(3-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,6-di azabicyclo[3.1.1]heptane-6-carbonyl)pyrrolidin-1-yl)phenyl)piperidine-2,6-dione

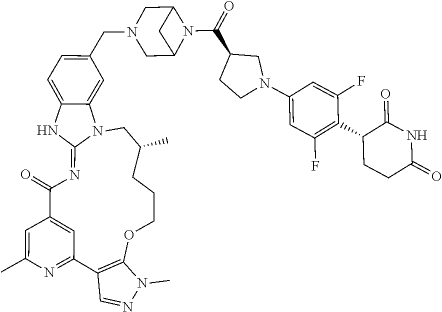 [SMILES: Cc1cc2cc(n1)-c1cnn(C)c1OCCC[C@@H](C)CN1/C(=N/C2=O)Nc2ccc(CN3CC4CC(C3)N4C(=O)[C@@H]3CCN(c4cc(F)c([C@H]5CCC(=O)NC5=O)c(F)c4)C3)cc21]

The titled compound was prepared in a manner similar to that in Example 5. ¹H NMR (500 MHz, DMSO) δ 12.70 (s, 1H), 10.84 (s, 1H), 8.42 (d, J=7.4 Hz, 1H), 7.92 (s, 1H), 7.57 (s, 1H), 7.49 (d, J=8.8 Hz, 2H), 7.18 (t, J=6.9 Hz, 1H), 6.21 (t, J=11.5 Hz, 2H), 4.52 (s, 1H), 4.35 (s, 1H), 4.21 (d, J=13.1 Hz, 2H), 4.01 (d, J=12.0 Hz, 2H), 3.95-3.88 (m, 1H), 3.88-3.76 (m, 2H), 3.73 (s, 3H), 3.47 (t, J=8.9 Hz, 1H), 3.36 (s, 2H), 3.22 (d, J=8.1 Hz, 1H), 3.14 (dd, J=14.4, 7.1 Hz, 1H), 3.08-2.89 (m, 3H), 2.82-2.62 (m, 3H), 2.56 (s, 3H), 2.48-2.42 (m, 2H), 2.22-1.84 (m, 7H), 1.78 (d, J=7.6 Hz, 1H), 1.44 (s, 1H), 0.80 (dd, J=16.5, 6.5 Hz, 3H); [M+H]⁺=861.7.

### Example 164: (3R)-3-(2,6-difluoro-4-((3R)-3-(5-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,5-di azabicyclo[2.2.2]octane-2-carbonyl)pyrrolidin-1-yl)phenyl)piperidine-2,6-dione

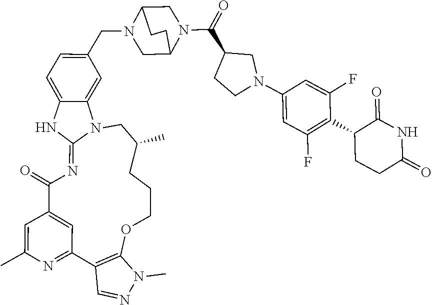 [SMILES: Cc1cc2cc(n1)-c1cnn(C)c1OCCC[C@@H](C)CN1/C(=N/C2=O)Nc2ccc(CN3CC4CCC3CN4C(=O)[C@@H]3CCN(c4cc(F)c([C@H]5CCC(=O)NC5=O)c(F)c4)C3)cc21]

The titled compound was prepared in a manner similar to that in Example 5. ¹H NMR (500 MHz, DMSO) δ 12.70 (s, 1H), 10.84 (s, 1H), 8.43 (s, 1H), 7.92 (s, 1H), 7.57 (s, 2H), 7.48 (d, J=8.1 Hz, 1H), 7.23 (d, J=8.1 Hz, 1H), 6.23 (t, J=12.9 Hz, 2H), 4.35 (s, 1H), 4.20 (d, J=12.1 Hz, 1H), 4.10-3.90 (m, 4H), 3.89-3.74 (m, 2H), 3.73 (s, 3H), 3.66 (d, J=11.5 Hz, 1H), 3.56-3.48 (m, 1H), 3.46-3.35 (m, 2H), 3.26 (d, J=13.2 Hz, 2H), 2.89-2.72 (m, 5H), 2.56 (s, 3H), 2.50-2.41 (m, 2H), 2.27-1.69 (m, 10H), 1.58 (s, 1H), 1.46 (s, 1H), 0.81 (d, J=6.1 Hz, 3H); [M+H]⁺=875.7.

### Example 165: (R)-3-(4-((R)-4-(4-(((R,E)-11-cyclopropyl-2⁶,7-dimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-)n-methyl )piperazine -1-carbonyl)-3,3-dimethylpyrrolidin-1-yl)-2,6-difluorophenyl)piperidine-2,6-dione

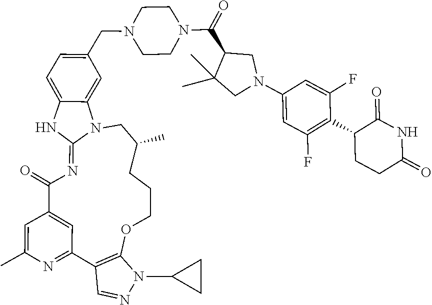 [SMILES: Cc1cc2cc(n1)-c1cnn(C3CC3)c1OCCC[C@@H](C)CN1/C(=N/C2=O)Nc2ccc(CN3CCN(C(=O)[C@H]4CN(c5cc(F)c([C@H]6CCC(=O)NC6=O)c(F)c5)CC4(C)C)CC3)cc21]

The titled compound was prepared in a manner similar to that in Example 5. ¹H NMR (500 MHz, DMSO) δ 12.72 (s, 1H), 10.84 (d, J=9.4 Hz, 1H), 8.48 (d, J=10.3 Hz, 1H), 7.89 (d, J=11.1 Hz, 1H), 7.55 (d, J=14.0 Hz, 2H), 7.50 (d, J=8.1 Hz, 1H), 7.20 (d, J=8.3 Hz, 1H), 6.14 (d, J=12.4 Hz, 2H), 4.41 (s, 1H), 4.21 (d, J=12.8 Hz, 1H), 4.12 (s, 1H), 4.06-3.90 (m, 2H), 3.77-3.59 (m, 5H), 3.53 (d, J=15.5 Hz, 2H), 3.43 (d, J=5.9 Hz, 5H), 3.07 (q, J=9.1 Hz, 2H), 2.90-2.74 (m, 2H), 2.57 (d, J=11.6 Hz, 5H), 2.39 (s, 3H), 2.26 (s, 1H), 2.16-1.99 (m, 2H), 1.95 (s, 2H), 1.49 (s, 1H), 1.17 (d, J=10.2 Hz, 4H), 1.11-1.02 (m, 3H), 0.97 (s, 2H), 0.82 (d, J=6.6 Hz, 2H). [M+H]⁺=903.4.

### Example 172: (R)-3-(2,6-difluoro-4-((R)-3-(4-(methyl((R,E)-1¹,2⁶,7-trimethyl-3-oxo-5²,5³-dihydro-1¹H,5¹H-11-oxa -4-aza-5(2,1)-benzo[d]imidazola-2(2,4)-pyridina-1(4,5)-pyrazolacycloundecaphane-5⁶-yl)amino)piperidine-1-carbonyl)pyrrolidin-1-yl)phenyl)piperidine-2,6-dione

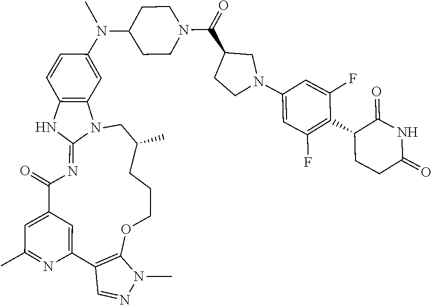 [SMILES: Cc1cc2cc(n1)-c1cnn(C)c1OCCC[C@@H](C)CN1/C(=N/C2=O)Nc2ccc(N(C)C3CCN(C(=O)[C@@H]4CCN(c5cc(F)c([C@H]6CCC(=O)NC6=O)c(F)c5)C4)CC3)cc21]

The titled compound was prepared in a manner similar to that in Example 5. ¹H NMR (500 MHz, DMSO) δ 12.46 (s, 1H), 10.84 (s, 1H), 8.43 (s, 1H), 7.92 (d, J=3.5 Hz, 1H), 7.56 (s, 1H), 7.37 (d, J=8.8 Hz, 1H), 7.00 (s, 1H), 6.86 (s, 1H), 6.23 (t, J=10.8 Hz, 2H), 4.55 (d, J=13.9 Hz, 1H), 4.37 (s, 1H), 4.16 (d, J=13.4 Hz, 2H), 3.99 (d, J=10.2 Hz, 5H), 3.73 (s, 3H), 3.57 (s, 1H), 3.01 (s, 1H), 2.75 (s, 8H), 2.55 (s, 6H), 2.19 (s, 3H), 1.95 (s, 3H), 1.73 (s, 3H), 1.47 (s, 2H), 0.83 (d, J=6.1 Hz, 3H). [M+H]⁺=863.8.

### Example 185: (R)-3-(4-((R)-3,3-dimethyl-4-((1S,4S)-5-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-2,5-diazabicyclo[2.2.1]heptane-2-carbonyl)pyrrolidin-1-yl)-2,6-difluorophenyl)piperidine-2,6-dione

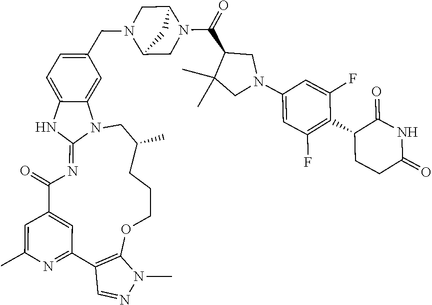 [SMILES: Cc1cc2cc(n1)-c1cnn(C)c1OCCC[C@@H](C)CN1/C(=N/C2=O)Nc2ccc(CN3C[C@@H]4C[C@H]3CN4C(=O)[C@H]3CN(c4cc(F)c([C@H]5CCC(=O)NC5=O)c(F)c4)CC3(C)C)cc21]

The titled compound was prepared in a manner similar to that in Example 5. ¹H NMR (500 MHz, DMSO) δ 12.70 (s, 1H), 10.84 (s, 1H), 8.42 (s, 1H), 7.92 (s, 1H), 7.58-7.46 (m, 3H), 7.20 (dd, J=21.6, 8.2 Hz, 1H), 6.15 (d, J=12.4 Hz, 2H), 4.62 (s, 1H), 4.36 (s, 1H), 4.20 (t, J=13.0 Hz, 1H), 4.05-3.78 (m, 6H), 3.73 (s, 3H), 3.70-3.44 (m, 5H), 3.16 (d, J=8.4 Hz, 3H), 2.95-2.73 (m, 4H), 2.56 (s, 3H), 2.22 (s, 1H), 2.10-1.82 (m, 5H), 1.74-1.60 (m, 1H), 1.47 (s, 1H), 1.26-0.97 (m, 6H), 0.81 (s, 3H). [M+H]⁺=889.7.

### Example 187: (3R)-3-(2,6-difluoro-4-((3R)-3-(8-(((R,E)-1¹,2,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)-3,8-di azabicyclo[3.2.1]octane-3-carbonyl)pyrrolidin-1-yl)phenyl)piperidine-2,6-dione

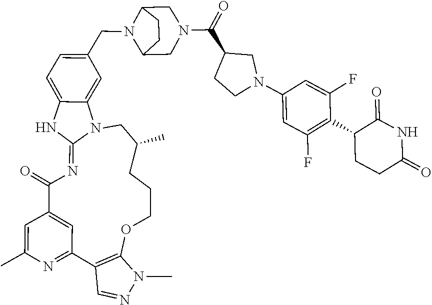 [SMILES: Cc1cc2cc(n1)-c1cnn(C)c1OCCC[C@@H](C)CN1/C(=N/C2=O)Nc2ccc(CN3C4CCC3CN(C(=O)[C@@H]3CCN(c5cc(F)c([C@H]6CCC(=O)NC6=O)c(F)c5)C3)C4)cc21]

The titled compound was prepared in a manner similar to that in Example 5. ¹H NMR (500 MHz, DMSO) δ 12.70 (s, 1H), 10.84 (s, 1H), 8.43 (s, 1H), 7.92 (s, 1H), 7.62 (s, 1H), 7.57 (s, 1H), 7.50 (d, J=8.0 Hz, 1H), 7.26 (d, J=7.3 Hz, 1H), 6.22 (dd, J=12.3, 7.0 Hz, 2H), 4.36 (s, 1H), 4.21 (d, J=12.6 Hz, 1H), 4.05-3.90 (m, 4H), 3.73 (s, 3H), 3.69-3.61 (m, 3H), 3.53-3.41 (m, 4H), 3.28-3.13 (m, 6H), 2.86-2.74 (m, 3H), 2.56 (s, 3H), 2.29-1.87 (m, 8H), 1.69-1.55 (m, 1H), 1.46 (d, J=6.8 Hz, 2H), 0.82 (d, J=4.5 Hz, 3H).; [M+H]⁺=875.7.

### Example 190: (R)-3-(4-((R)-2,2-dimethyl-3-((S)-3-methyl-4-(((R,E)-1¹,2⁶,7-trimethyl-3-oxo-5²,5³-dihydro-1¹H,5¹H -11-oxa-4-aza-5(2,1)-benzo[d]imidazola-2(2,4)-pyridina-(4,5)-pyrazolacycloundecaphane-5⁶-yl)methyl)piperazine-1-carbonyl)pyrrolidin-1-yl)-2,6-difluorophenyl)piperidine-2,6-dione

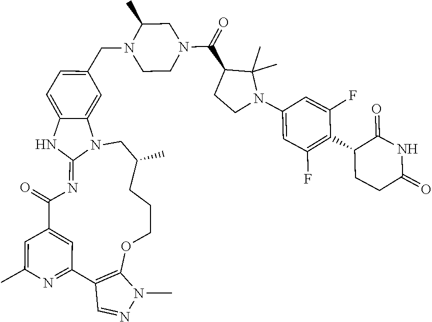 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4(C)C)C[C@@H]3C)cc21]

The titled compound was prepared in a manner similar to that in Example 5. ¹H NMR (500 MHz, DMSO) δ 12.71 (s, 1H), 10.83 (s, 1H), 8.43 (s, 1H), 7.92 (s, 1H), 7.57 (s, 1H), 7.53 (s, 1H), 7.49 (d, J=8.2 Hz, 1H), 7.20 (d, J=7.8 Hz, 1H), 6.14 (d, J=13.2 Hz, 2H), 4.37 (s, 1H), 4.21 (d, J=12.1 Hz, IH), 4.03-3.95 (m, 5H), 3.81 (d, J=13.6 Hz, 2H), 3.73 (s, 3H), 3.42 (d, J=14.1 Hz, 6H), 3.08 (dd, J=12.3, 7.1 Hz, 2H), 2.81-2.73 (m, 2H), 2.63 (s, 1H), 2.56 (s, 3H), 2.21 (s, 1H), 2.08 (d, J=11.1 Hz, 1H), 2.04-1.92 (m, 4H), 1.48 (s, 1H), 1.21-1.05 (m, 7H), 0.96 (d, J=7.8 Hz, 3H), 0.81 (d, J=5.8 Hz, 3H), [M+H]⁺=891.7.

### Example 194: (R)-3-(2,6-difluoro-4-((R)-3-((R)-3-(methoxymethyl)-4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5′-yl)methyl)piperazine-1-carbonyl)pyrrolidin-1-yl)phenyl)piperidine-2,6-dione

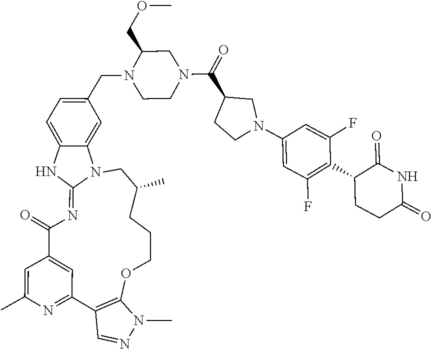 [SMILES: COC[C@H]1CN(C(=O)[C@@H]2CCN(c3cc(F)c([C@H]4CCC(=O)NC4=O)c(F)c3)C2)CCN1Cc1ccc2c(c1)N1C[C@H](C)CCCOc3c(cnn3C)-c3cc(cc(C)n3)C(=O)/N=C/1N2]

The titled compound was prepared in a manner similar to that in Example 5. ¹H NMR (500 MHz, DMSO) δ 12.71 (s, 1H), 10.84 (s, 1H), 8.43 (s, 1H), 7.92 (s, 1H), 7.56 (d, J=9.9 Hz, 2H), 7.49 (d, J=8.1 Hz, 1H), 7.22 (d, J=8.2 Hz, 1H), 6.22 (d, J=12.3 Hz, 2H), 4.36 (d, J=4.8 Hz, 1H), 4.22 (d, J=11.9 Hz, 1H), 4.00 (s, 3H), 3.91 (dd, J=21.9, 9.8 Hz, 2H), 3.73 (s, 3H), 3.72-3.70 (m, 1H), 3.65 (d, J=5.5 Hz, 1H), 3.63-3.55 (m, 2H), 3.51 (s, 2H), 3.44 (dd, J=16.0, 8.6 Hz, 2H), 3.34 (s, 2H), 3.26 (d, J=19.6 Hz, 5H), 3.20-3.08 (m, 1H), 2.84-2.74 (m, 2H), 2.71 (d, J=16.9 Hz, 1H), 2.56 (s, 3H), 2.21 (s, 2H), 2.13 (s, 1H), 2.10-2.01 (m, 2H), 2.01-1.86 (m, 3H), 1.47 (s, 1H), 0.82 (d, J=6.2 Hz, 3H). [M+H]⁺=893.4.

### Example 195: (R)-3-(2,6-difluoro-4-((R)-3-((R)-3-(hydroxymethyl)-4-(((R,E)-1¹,2⁵,7-trimethyl-3-oxo-5²,5³-dihydro-1¹H,5¹H-11-oxa-4-aza-5(2,1)-benzo[d]imidazola-2(2,4)-pyridina-(4,5)-pyrazolacycloundecaphane-5⁶-yl)methyl)piperazine-1-carbonyl)pyrrolidin-1-yl)phenyl)piperidine-2,6-dione

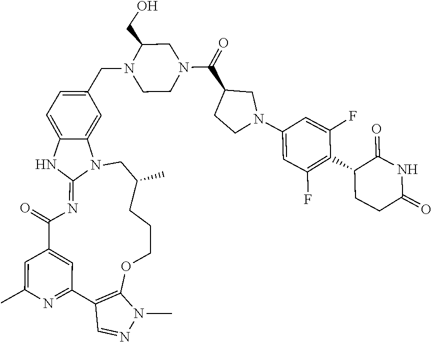 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)C[C@@H]3CO)cc21]

The titled compound was prepared in a manner similar to that in Example 5. ¹H NMR (500 MHz, DMSO) δ 12.70 (s, 1H), 10.84 (s, 1H), 8.43 (s, 1H), 7.92 (s, 1H), 7.57 (s, 1H), 7.56 (d, J=8.0 Hz, 1H), 7.49 (d, J=8.0 Hz, 1H), 7.22 (d, J=7.5 Hz, 1H), 6.21 (d, J=13.0 Hz, 2H), 4.76-4.63 (m, 1H), 4.42-4.31 (m, 1H), 4.23-3.89 (m, 8H), 3.73 (s, 3H), 3.57-3.42 (m, 8H), 3.07-2.98 (m, 1H), 2.84-2.66 (m, 5H), 2.56 (s, 3H), 2.26-1.89 (m, 7H), 1.53-1.40 (m, 1H), 0.82 (d, J=5.0 Hz, 3H); [M+H]⁺=879.7.

### Example 196: (R)-3-(2,6-difluoro-4-((R)-3-((S)-3-(hydroxymethyl)-4-(((R,E)-1¹,2⁶,7-trimethyl-3-oxo-5²,5³-dihydro -1¹H,5¹H-11-oxa-4-aza-5(2,1)-benzo[d]imidazola-2(2,4)-pyridina-1(4,5)-pyrazolacycloundecaphane-56-yl)methyl)piperazine-1-carbonyl)pyrrolidin-1-yl)phenyl)piperidine-2,6-dione

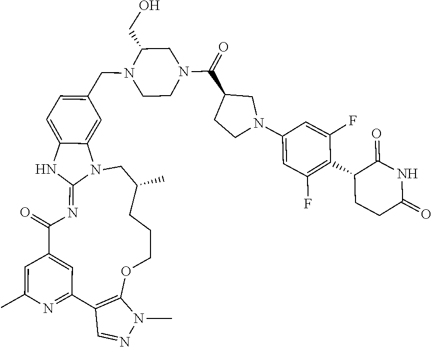 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)C[C@H]3CO)cc21]

The titled compound was prepared in a manner similar to that in Example 5. ¹H NMR (500 MHz, DMSO) δ 12.70 (s, 1H), 10.84 (s, 1H), 8.43 (s, 1H), 7.92 (s, 1H), 7.57 (s, 1H), 7.56 (d, J=8.0 Hz, 1H), 7.49 (d, J=8.0 Hz, 1H), 7.22 (d, J=7.5 Hz, 1H), 6.21 (d, J=13.0 Hz, 2H), 4.76-4.63 (m, 1H), 4.42-4.31 (m, 1H), 4.23-3.89 (m, 8H), 3.73 (s, 3H), 3.57-3.42 (m, 8H), 3.07-2.98 (m, 1H), 2.84-2.66 (m, 5H), 2.56 (s, 3H), 2.26-1.89 (m, 7H), 1.53-1.40 (m, 1H), 0.82 (d, J=5.0 Hz, 3H); [M+H]⁺=879.7.

### Example 199: (R)-3-(2,6-difluoro-4-((R)-3-(5-(((R,E)-1¹,2⁶,7-trimethyl-3-oxo-5²,5³-dihydro-1¹H,5¹H-11-oxa-4-aza -5(2,1)-benzo[d]imidazola-2(2,4)-pyridina-1(4,5)-pyrazolacycloundecaphane-5⁶-yl)methyl)-2-oxa-5,8-diazaspiro[3.5]nonane-8-carbonyl)pyrrolidin-1-yl)phenyl)piperidine-2,6-dione

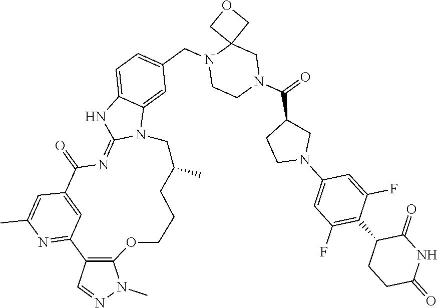 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC34COC4)cc21]

The titled compound was prepared in a manner similar to that in Example 5. ¹H NMR (500 MHz, DMSO) δ 12.72 (s, 1H), 10.84 (d, J=2.8 Hz, 1H), 8.44 (s, 1H), 8.00-7.90 (m, 1H), 7.63-7.56 (m, 2H), 7.54-7.46 (m, 1H), 7.27 (d, J=8.4 Hz, 1H), 6.23 (d, J=12.7 Hz, 2H), 4.72 (d, J=9.6 Hz, 2H), 4.37 (s, 3H), 4.22 (d, J=15.2 Hz, 3H), 4.01 (s, 5H), 3.95 (s, 2H), 3.86 (d, J=17.0 Hz, 1H), 3.77-3.70 (m, 4H), 3.53 (s, 3H), 2.80 (d, J=14.4 Hz, 4H), 2.37 (s, 1H), 2.23 (s, 2H), 2.07 (s, 3H), 1.98 (d. J=30.2 Hz, 4H), 1.48 (s, 1H), 0.83 (d, J=6.4 Hz, 4H). [M+H]⁺=891.4

### Example 200: (R)-3-(2,6-difluoro-4-((R)-3-(3-oxo-4-(((R,E)-1¹,2⁶,7-trimethyl-3-oxo-5²,5³-dihydro-1¹H,5¹H-11-oxa -4-aza-5(2,1)-benzo[d]imidazola-2(2,4)-pyridina-1(4,5)-pyrazolacycloundecaphane-5⁶-yl)methyl)piperazine-1-carbonyl)pyrrolidin-1-yl)phenyl)piperidine-2,6-dione

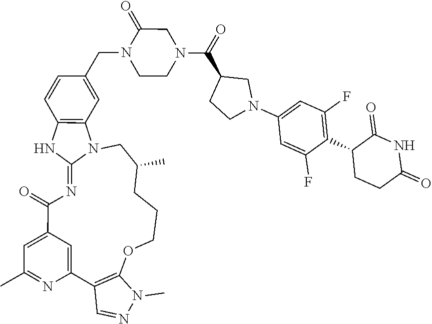 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3=O)cc21]

The titled compound was prepared in a manner similar to that in Example 5. ¹H NMR (500 MHz, DMSO) δ 12.74 (s, 1H), 10.84 (s, 1H), 8.43 (s, 1H), 7.92 (s, 1H), 7.57 (s, 1H), 7.52 (d, J=8.0 Hz 1H), 7.48 (s, 1H), 7.47 (s, 1H), 7.16 (d, J=8.0 Hz, 1H), 6.26-6.12 (m, 2H), 4.80-4.56 (m, 2H), 4.39-4.34 (m, 2H), 4.23-3.81 (m, 6H), 3.73 (s, 3H), 3.71-3.21 (m, 9H), 2.82-2.73 (m, 2H), 2.56 (s, 3H), 2.26-1.87 (m, 7H), 1.50-1.43 (m, 1H), 0.82 (s, 1H), 0.80 (s, 1H); [M+H]⁺=863.7.

### Example 202: (R)-3-(2,6-difluoro-4-((R)-3-((S)-2-(methoxymethyl)-4-(((R,E)-1¹,2⁶,7-trimethyl-3-oxo-5²,5³-dihydro-1¹H,5¹H-li-oxa-4-aza-5(2,1)-benzo[d]imidazola-2(2,4)-pyridina-(4,5)-pyrazolacycloundecaphane-5⁶-yl)methyl)piperazine-1-carbonyl)pyrrolidin-1-yl)phenyl)piperidine-2,6-dione

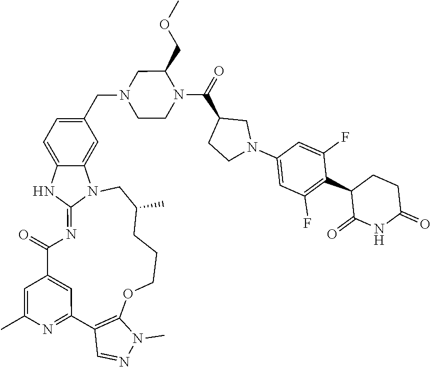 [SMILES: COC[C@@H]1CN(Cc2ccc3c(c2)N2C[C@H](C)CCCOc4c(cnn4C)-c4cc(cc(C)n4)C(=O)/N=C/2N3)CCN1C(=O)[C@@H]1CCN(c2cc(F)c([C@H]3CCC(=O)NC3=O)c(F)c2)C1]

The titled compound was prepared in a manner similar to that in Example 5. ¹H NMR (500 MHz, DMSO) δ 12.73 (s, 1H), 10.83 (s, 1H), 8.43 (s, 1H), 7.92 (s, 1H), 7.57 (s, 1H), 7.54-7.47 (m, 2H), 7.21-7.15 (m, 1H), 6.26-6.14 (m, 2H), 4.40-4.32 (m, 1H), 4.30-4.16 (m, 2H), 4.05-3.91 (m, 3H), 3.90-3.80 (m, 1H), 3.74-3.67 (m, 4H), 3.58-3.48 (m, 2H), 3.44-3.34 (m, 3H), 3.30-3.19 (m, 6H), 2.96-2.69 (m, 5H), 2.56 (s, 3H), 2.27-1.86 (m, 1 OH), 1.46 (s, 1H), 0.84 (t, J=7.2 Hz, 3H): [M+H]⁺=893.7.

### Example 256: (R,E)-N-((3R,4S)-1-(4-((R)-2,6-dioxopiperidin-3-yl)-3,5-difluorophenethyl)-3-methylpiperidin-4-yl) -N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

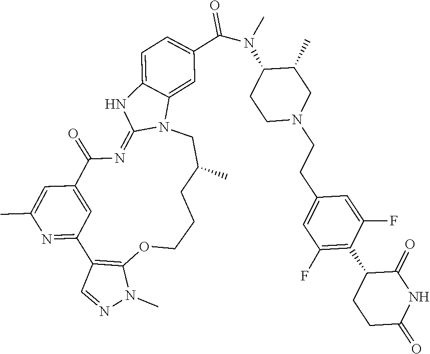 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)C[C@H]3C)cc21]

The titled compound was prepared in a manner similar to that in Example 260.

¹H NMR (500 MHz, DMSO) δ 12.83 (s, 1H), 10.93 (s, 1H), 8.43 (s, 1H), 7.93 (s, 1H), 7.67 (s, 1H), 7.57 (s, 2H), 7.22 (d, J=8.2 Hz, 1H), 7.01 (d, J=10.0 Hz, 2H), 4.36 (d, J=4.3 Hz, 1H), 4.20 (d, J=10.7 Hz, 2H), 4.06-4.00 (m, 2H), 3.73 (s, 3H), 2.98 (s, 1H), 2.89 (s, 3H), 2.82-2.77 (m, 3H), 2.70 (s, 3H), 2.56 (s, 4H), 2.52 (s, 2H), 2.36 (s, 2H), 2.21 (s, 1H), 2.15-2.04 (m, 3H), 2.03-1.87 (m, 3H), 1.60 (d, J=9.3 Hz, 1H), 1.45 (s, 1H), 1.06 (d, J=6.7 Hz, 3H), 0.82 (d, J=6.2 Hz, 3H), [M+H]⁺=836.6.

### Example 211: 3-(7-fluoro-3-methyl-2-oxo-4-((R)-3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)p iperazine-1-carbonyl)pyrrolidin-1-yl)-2,3-dihydro-1H-benzo[d]imidazol-1-yl)piperidine-2,6-dione

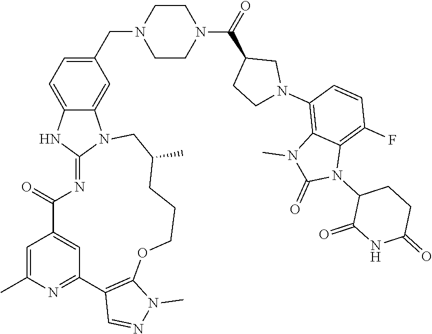 [SMILES: Cc1cc2cc(n1)-c1cnn(C)c1OCCC[C@@H](C)CN1/C(=N/C2=O)Nc2ccc(CN3CCN(C(=O)[C@@H]4CCN(c5ccc(F)c6c5n(C)c(=O)n6C5CCC(=O)NC5=O)C4)CC3)cc21]

The titled compound was prepared in a manner similar to that in Example 5. ¹H NMR (500 MHz, DMSO) 5=12.71 (s, 1H), 11.10 (s, 1H), 8.43 (s, 1H), 7.92 (s, 1H), 7.57 (s, 1H), 7.53 (s, 1H), 7.49 (d, J=8.1, 1H), 7.19 (d, J=7.8, 1H), 7.00 (m, 1H), 6.88 (t, J=10.0, 1H), 5.50 (d, J=8.3, 1H), 4.36 (m, 1H), 4.20 (d, J=12.4, 1H), 4.00 (s, 1H), 3.98-3.89 (m, 1H), 3.73 (s, 3H), 3.62 (d, J=12.7, 5H), 3.52 (s, 4H), 3.44 (m, 1H), 3.18 (d, J=6.0, 2H), 3.03 (m, 3H), 2.81 (s, 1H), 2.63 (d, J=20, 2H), 2.38 (m, 6H), 2.25-1.87 (m, 7H), 1.46 (s, 1H), 0.81 (d, J=6.2, 3H); [M+H]⁺=901.8.

### Example 214: (R)-3-(2,6-difluoro-4-(4-(methyl((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)amino)-[1,4′-bipiperidin]-1′-yl)phenyl)piperidine-2,6-dione

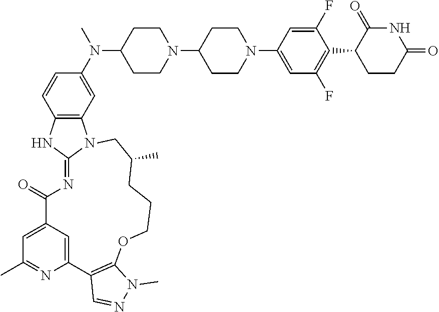 [SMILES: Cc1cc2cc(n1)-c1cnn(C)c1OCCC[C@@H](C)CN1/C(=N/C2=O)Nc2ccc(N(C)C3CCN(C4CCN(c5cc(F)c([C@H]6CCC(=O)NC6=O)c(F)c5)CC4)CC3)cc21]

The titled compound was prepared in a manner similar to that in Example 14. ¹H NMR (500 MHz, DMSO) δ 12.44 (s, 1H), 10.86 (s, 1H), 8.42 (s, 1H), 8.24 (s, 1H), 7.92 (d, J=5.3 Hz, 1H), 7.55 (s, 1H), 6.93 (s, 1H), 6.78 (d, J=8.3 Hz, 1H), 6.63 (d, J=12.7 Hz, 2H), 4.37 (s, 1H), 4.15 (d, J=15.2 Hz, 1H), 4.00 (m, 4H), 3.81 (d, J=12.9 Hz, 3H), 3.73 (s, 3H), 3.60 (s, 1H), 2.95 (s, 2H), 2.75 (s, 4H), 2.55 (s, 6H), 2.29 (s, 3H), 2.07 (s, 1H), 1.97 (s, 2H), 1.80 (d, J=10.8 Hz, 2H), 1.65 (s, 5H), 1.49 (s, 3H), 0.82 (d, J=6.1 Hz, 3H). [M+H]⁺=849.7.

### Example 216: 2-(2,6-dioxopiperidin-3-yl)-5-((R)-3-(4-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperazine-1-carbonyl)pyrrolidin-1-yl)isoindoline-1,3-dione

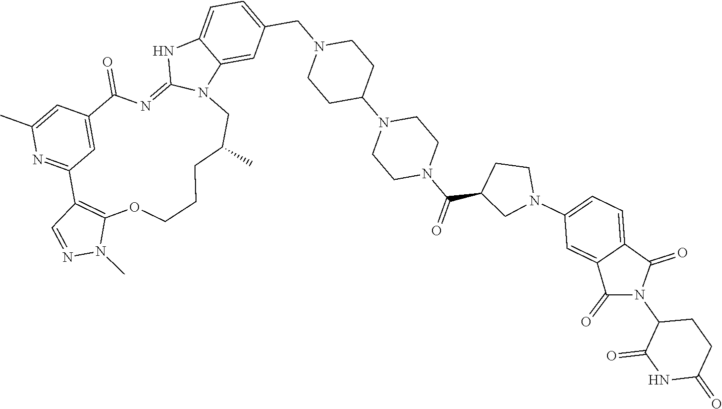 [SMILES: Cc1cc2cc(n1)-c1cnn(C)c1OCCC[C@@H](C)CN1/C(=N/C2=O)Nc2ccc(CN3CCC(N4CCN(C(=O)[C@H]5CCN(c6ccc7c(c6)C(=O)N(C6CCC(=O)NC6=O)C7=O)C5)CC4)CC3)cc21]

The titled compound was prepared in a manner similar to that in Example 5. ¹H NMR (500 MHz, DMSO) δ 12.73 (s, 1H), 11.06 (s, 1H), 8.43 (s, 1H), 7.92 (s, 1H), 7.64 (d, J=8.4 Hz, 1H), 7.57 (s, 1H), 7.50 (s, 2H), 7.19 (s, 1H), 6.92 (s, 1H), 6.83 (d, J=8.6 Hz, 1H), 5.05 (dd, J=12.9, 5.4 Hz, 1H), 4.36 (d, J=5.1 Hz, 1H), 4.21 (d, J=12.2 Hz, 1H), 4.00 (s, 1H), 3.94 (d, J=11.3 Hz, 1H), 3.73 (s, 3H), 3.65-3.53 (m, 5H), 3.51-3.43 (m, 5H), 2.96-2.84 (m, 2H), 2.83-2.81 (m, 2H), 2.67-2.53 (m, 6H), 2.49-2.42 (m, 2H), 2.22 (d, J=6.3 Hz, 3H), 2.12 (d, J=7.4 Hz, 1H), 2.04-1.84 (m, 4H), 1.75 (s, 2H), 1.46 (s, 3H), 0.82 (d, J=6.4 Hz, 3H), 0.82 (d, J=6.4 Hz, 3H). [M+H]⁺=965.6

### Example 221: (R)-3-(2,6-difluoro-4-(3-(7-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-carbonyl)-2,7-diazaspiro nonane-2-carbonyl)azetidin-1-yl)phenyl)piperidine-2,6-dione

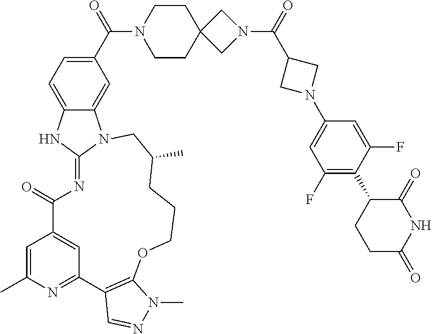 [SMILES: Cc1cc2cc(n1)-c1cnn(C)c1OCCC[C@@H](C)CN1/C(=N/C2=O)Nc2ccc(C(=O)N3CCC4(CC3)CN(C(=O)C3CN(c5cc(F)c([C@H]6CCC(=O)NC6=O)c(F)c5)C3)C4)cc21]

The titled compound was prepared in a manner similar to that in Example 96. ¹H NMR (500 MHz, DMSO) δ 12.86 (s, 1H), 10.86 (s, 1H), 8.43 (s, 1H), 7.93 (s, 1H), 7.67 (s, 1H), 7.58-7.56 (m, 2H), 7.25 (d, J=8.0 Hz, 1H), 6.14 (d, J=11.5 Hz, 2H), 4.38-4.34 (m, 1H), 4.21-4.17 (m, 1H), 4.05-3.97 (m, 5H), 3.85-3.83 (m, 4H), 3.73 (s, 3H), 3.66 (s, 2H), 3.59-3.53 (m, 2H), 2.88-2.70 (m, 2H), 2.56 (s, 3H), 2.50-2.44 (m, 4H), 2.28-2.18 (m, 1H), 2.10-1.89 (m, 4H), 1.82-1.68 (m, 4H), 1.53-1.39 (m, 1H), 0.82 (d, J=6.5 Hz, 3H); [M+H]⁺=889.8.

### Example 229: (R)-3-(2,6-difluoro-4-(3-(6-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)-2,6-diazaspiro heptane-2-carbonyl)azetidin-1-yl)phenyl)piperidine-2,6-dione

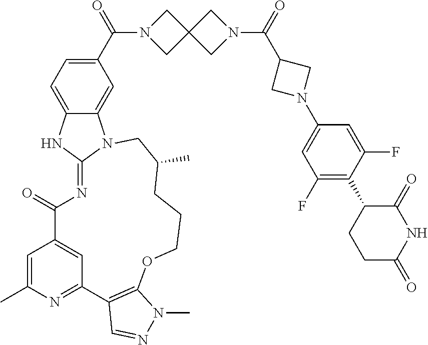 [SMILES: Cc1cc2cc(n1)-c1cnn(C)c1OCCC[C@@H](C)CN1/C(=N/C2=O)Nc2ccc(C(=O)N3CC4(C3)CN(C(=O)C3CN(c5cc(F)c([C@H]6CCC(=O)NC6=O)c(F)c5)C3)C4)cc21]

The titled compound was prepared in a manner similar to that in Example 96. ¹H NMR (500 MHz, DMSO) δ 12.91 (s, 1H), 10.85 (s, 1H), 8.44 (s, 1H), 7.93 (s, 1H), 7.82 (s, 1H), 7.58-7.57 (m, 2H), 7.52 (d. .=8.0 Hz, 1H), 6.14 (d, J=16.0 Hz, 2H), 4.56-4.50 (m, 2H), 4.40-4.33 (m, 1H), 4.28-4.19 (m, 5H), 4.08-3.97 (m, 7H), 3.86-3.79 (m, 2H), 3.73 (s, 3H), 3.57-3.50 (m, 1H), 2.85-2.72 (m, 2H), 2.57 (s, 3H), 2.27-2.17 (m, 1H), 2.12-1.89 (m, 5H), 1.53-1.39 (m, 1H), 0.83 (d, J=6.5 Hz, 3H), [M+H]⁺=861.7.

### Example 232: (R,E)-N-(1-(4-((R)-2,6-dioxopiperidin-3-yl)-3,5-difluorophenethyl)piperidin-4-yl)-5⁵-fluoro-N,1¹,2⁶, 7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

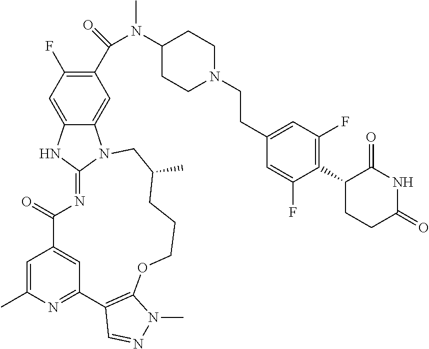 [SMILES: Cc1cc2cc(n1)-c1cnn(C)c1OCCC[C@@H](C)CN1/C(=N/C2=O)Nc2cc(F)c(C(=O)N(C)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.90 (s, 1H), 10.93 (d, J=12.3 Hz, 1H), 8.42 (s, 1H), 7.92 (s, 1H), 7.68 (d, J=5.5 Hz, 1H), 7.57 (s, 1H), 7.42-7.32 (m, 1H), 7.04 (d, J=9.9 Hz, 1H), 6.95 (s, 1H), 4.34-4.32 (m, 1H), 4.24-4.11 (m, 2H), 4.05-3.94 (m, 2H), 3.73 (s, 3H), 3.29 (s, 1H), 3.11-3.02 (m, 1H), 2.92 (s, 2H), 2.79-2.77 (m, 3H), 2.69 (s, 2H), 2.65-2.63 (m, 1H), 2.61-2.52 (m, 5H), 2.45-2.34 (m, 2H), 2.27-2.16 (m, 1H), 2.15-2.04 (m, 2H), 2.03-1.87 (m, 3H), 1.87-1.73 (m, 2H), 1.69-1.60 (m, 1H), 1.59-1.51 (m, 1H), 1.44-1.42 (m, H), 0.85-0.75 (m, 3H); [M+H]⁺=840.7.

### Example 233: (R,E)-N-(1-(4-((R)-2,6-dioxopiperidin-3-yl)-3,5-difluorophenethyl)piperidin-4-yl)-N,1¹,1³,2⁶,7-penta methyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

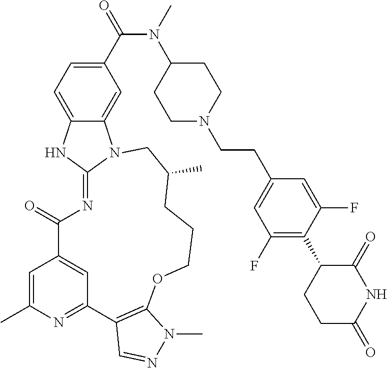 [SMILES: Cc1cc2cc(n1)-c1cnn(C)c1OCCC[C@@H](C)CN1/C(=N/C2=O)Nc2ccc(C(=O)N(C)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.85 (s, 1H), 10.93 (s, 1H), 8.43 (s, 1H), 7.66 (s, 1H), 7.57 (d, J=8.1 Hz, 1H), 7.54 (s, 1H), 7.22 (s, 1H), 6.98 (s, 2H), 4.34 (d, J=2.9 Hz, 1H), 4.23-4.13 (m, 2H), 4.05-3.94 (m, 2H), 3.66 (s, 3H), 3.28-3.21 (m, 2H), 3.09-2.62 (m, 10H), 2.56 (s, 3H), 2.53 (d, J=6.4 Hz, 2H), 2.49-2.35 (m, 2H), 2.27-2.05 (m, 3H), 2.02-1.88 (m, 3H), 1.79 (dd, J=19.6, 11.0 Hz, 2H), 1.72-1.51 (m, 3H), 1.43 (d, J=6.4 Hz, 1H), 0.82 (d, J=6.4 Hz, 3H). [M+H]⁺=836.7

### Example 234: (E)-N-(1-(4-((R)-2,6-dioxopiperidin-3-yl)-3,5-difluorophenethyl)piperidin-4-yl)-N,1¹,2⁶-trimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-carboxamide

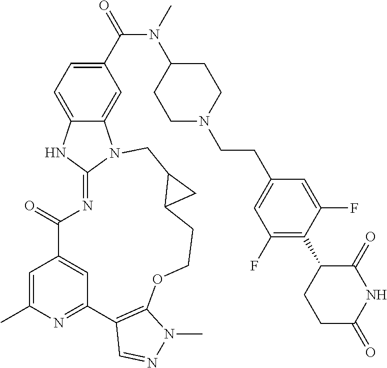 [SMILES: Cc1cc2cc(n1)-c1cnn(C)c1OCCC1CC1CN1/C(=N/C2=O)Nc2ccc(C(=O)N(C)C3CCN(CCc4cc(F)c([C@H]5CCC(=O)NC5=O)c(F)c4)CC3)cc21]

The titled compound was prepared in a manner similar to that in Example 260.

[M+H]⁺=820.7.

### Example 236: (R,E)-N-(1-(4-(2,6-dioxopiperidin-3-yl)-3,5-difluorophenethyl)piperidin-4-yl)-N,1′,6′-trimethyl-3′-oxospiro[cyclopropane-1,7′-11-oxa-4-aza-5(2,1)-benzo[d]imidazola-2(2,4)-pyridina-1(4,5)-pyrazolacycloundecaphane]-6′-carboxamide

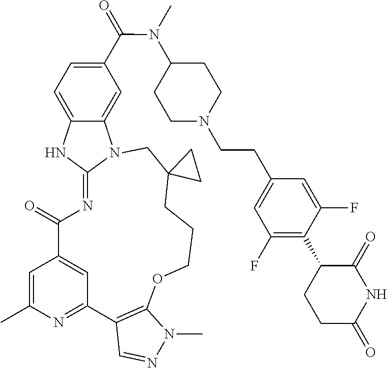 [SMILES: Cc1cc2cc(n1)-c1cnn(C)c1OCCCC1(CC1)CN1/C(=N/C2=O)Nc2ccc(C(=O)N(C)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 13.01 (s, 1H), 10.94 (s, 1H), 8.34 (s, 1H), 7.91 (s, 1H), 7.58 (s, 2H), 7.53 (s, 1H), 7.23 (s, 1H), 6.98 (s, 2H), 4.29-4.17 (m, 6H), 3.73 (s, 3H), 3.10-2.61 (m, 10H), 2.56 (s, 3H), 2.24 (s, 2H), 2.11 (d, J=10.7 Hz, 2H), 1.99 (s, 1H), 1.90-1.77 (m, 4H), 1.76-1.63 (m, 4H), 0.70 (s, 2H), 0.40 (s, 2H); [M+H]⁺=834.4.

### Example 238: (R,E)-N-(1-(4-(2,6-dioxopiperidin-3-yl)-3,5-difluorophenethyl)piperidin-4-yl)-N,1¹,2⁶-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

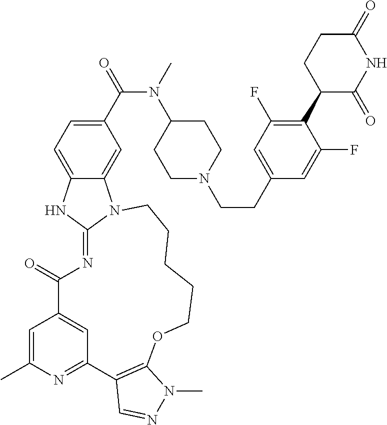 [SMILES: Cc1cc2cc(n1)-c1cnn(C)c1OCCCCCN1/C(=N/C2=O)Nc2ccc(C(=O)N(C)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) h 12.80 (s, 1H), 10.94 (s, 1H), 8.41 (s, 1H), 7.92 (s, 1H), 7.67 (s, 1H), 7.60-7.49 (m, 2H), 7.24 (d, J=7.4 Hz, 1H), 7.00 (s, 2H), 4.20 (t, J=11.0 Hz, 5H), 3.73 (s, 3H), 3.11-2.61 (m, 9H), 2.55 (d, J=13.5 Hz, 5H), 2.08 (d, J=30.9 Hz, 8H), 1.72 (d, J=65.5 Hz, 7H). [M+H]⁺=808.9.

### Example 239: (R,E)-N-cyclopropyl-N-(1-(4-((R)-2,6-dioxopiperidin-3-yl)-3,5-difluorophenethyl)piperidin-4-yl)-1¹, 2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

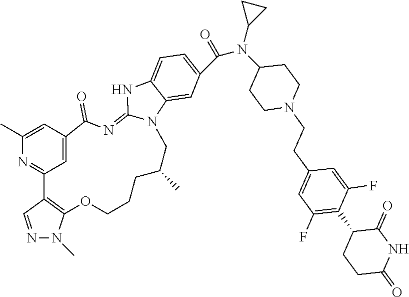 [SMILES: Cc1cc2cc(n1)-c1cnn(C)c1OCCC[C@@H](C)CN1/C(=N/C2=O)Nc2ccc(C(=O)N(C3CC3)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.80 (s, 1H), 10.95 (s, 1H), 8.43 (s, 1H), 7.93 (s, 1H), 7.75 (s, 1H), 7.57 (s, 1H), 7.53 (d, J=8.1 Hz, 1H), 7.34 (d, J=8.1 Hz, 1H), 7.03 (d, J=10.1 Hz, 2H), 4.36 (s, 1H), 4.20 (d. .=12.2 Hz, 2H), 4.04-4.01 (m, 3H), 3.73 (s, 3H), 3.04 (s, 2H), 2.78 (d, J=6.6 Hz, 5H), 2.56-2.53 (m, 6H), 2.21 (s, 1H), 2.16-1.90 (m, 8H), 1.86 (s, 2H), 1.44 (s, 1H), 0.80 (d, J=6.2 Hz, 3H), 0.53-0.46 (m, 2H), 0.34-0.32 (m, 2H); [M+H]⁺=848.5.

### Example 241: (R,E)-N-(1-(4-((R)-2,6-dioxopiperidin-3-yl)-3,5-difluorophenethyl)piperidin-4-yl)-N-ethyl-1¹,2⁶, 7-trimethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁴-carboxamide

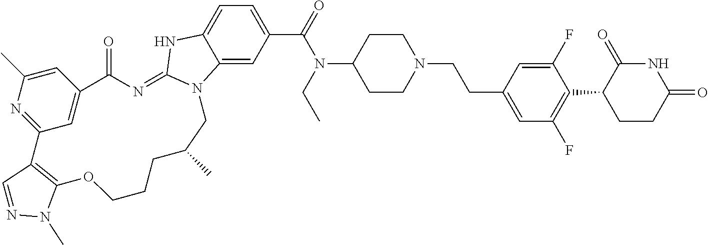 [SMILES: CCN(C(=O)c1ccc2c(c1)N1C[C@H](C)CCCOc3c(cnn3C)-c3cc(cc(C)n3)C(=O)/N=C/1N2)C1CCN(CCc2cc(F)c([C@H]3CCC(=O)NC3=O)c(F)c2)CC1]

The titled compound was prepared in a manner similar to that in Example 260.

¹H NMR (500 MHz, DMSO) δ 12.85 (s, 1H), 10.93 (s, 1H), 8.43 (s, 1H), 7.93 (s, 1H), 7.59 (t, J=18.7 Hz, 3H), 7.17 (d, J=7.9 Hz, 1H), 6.97 (s, 2H), 4.37 (s, 1H), 4.19 (d, J=11.0 Hz, 2H), 4.03 (dd, J=17.0, 12.4 Hz, 2H), 3.73 (s, 3H), 3.37 (s, 2H), 2.90 (s, 2H), 2.78 (d, J=12.2 Hz, 2H), 2.66 (s, 2H), 2.56 (s, 3H), 2.53 (s, 2H), 2.42 (s, 3H), 2.21 (s, 1H), 2.10 (d, J=11.5 Hz, 1H), 2.03-1.89 (m, 3H), 1.63 (dd, J=11.0, 3.0 Hz, 7H), 1.18 (s, 2H), 0.82 (d, J=6.1 Hz, 3H), [M+H]⁺=837.0.

### Example 242: (R,E)-N-(1-(4-((R)-2,6-dioxopiperidn-3-yl)-3,5-difluorophenethyl)piperidin-4-yl)-1¹,2⁶,7-trimethyl-3-oxo-5²,5′-dihydro-1¹H,5¹H-li-oxa-4-aza-5(2,1)-benzo[d]imidazola-2(2,4)-pyridina-1(4,5)-pyrazolacycloundecaphane-5⁶-carboxamide

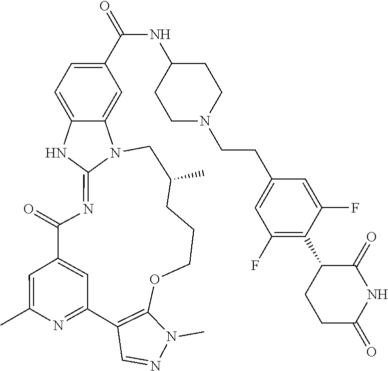 [SMILES: Cc1cc2cc(n1)-c1cnn(C)c1OCCC[C@@H](C)CN1/C(=N/C2=O)Nc2ccc(C(=O)NC3CCN(CCc4cc(F)c([C@H]5CCC(=O)NC5=O)c(F)c4)CC3)cc21]

The titled compound was prepared in a manner similar to that in Example 260. 1H NMR (500 MHz, DMSO) δ 12.89 (s, 1H), 10.95 (s, 1H), 8.43 (s, 1H), 8.26-8.15 (m, 1H), 8.06 (s, 1H), 7.93 (s, 1H), 7.78 (d, J=7.7 Hz, 1H), 7.62-7.52 (m, 2H), 7.04 (d, J=10.1 Hz, 2H), 4.37 (s, 1H), 4.22 (d, J=11.9 Hz, 2H), 4.03-3.92 (m, 2H), 3.83 (s, 2H), 3.74 (s, 3H), 2.98 (d, J=10.2 Hz, 2H), 2.90-2.75 (m, 5H), 2.64 (s, 1H), 2.56 (s, 3H), 2.22 (s, 1H), 2.14 (d, J=13.5 Hz, 1H), 2.10-1.97 (m, 4H), 1.92 (s, 1H), 1.83 (d, J=11.1 Hz, 2H), 1.60 (s, 2H), 1.49 (s, 1H), 0.83 (d, J=6.1 Hz, 3H). [M+H]⁺=808.7

### Example 243: (R,E)-N-((3R,4R)-1-(4-((R)-2,6-dioxopiperidin-3-yl)-3,5-difluorophenethyl)-3-hydroxypiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4) -pyridina-1(4,5)-pyrazolacycloundecaphane-5⁶-carboxamide

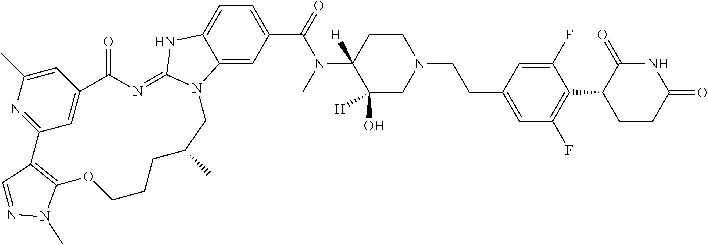 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3O)cc21]

The titled compound was prepared in a manner similar to that in Example 260.

¹H NMR (500 MHz, DMSO) δ 12.84 (s, 1H), 10.93 (s, 1H), 8.43 (s, 1H), 7.93 (s, 1H), 7.66 (s, 1H), 7.56 (d, J=12.2 Hz, 2H), 7.25 (dd, J=19.2, 8.0 Hz, 1H), 7.00 (dd, J=18.0, 10.2 Hz, 2H), 5.09 (s, 1H), 4.36 (s, 1H), 4.19 (t, J=14.6 Hz, 2H), 3.98 (d, J=17.4 Hz, 2H), 3.73 (s, 3H), 3.18 (d, J=12.4 Hz, 1H), 2.99 (s, 1H), 2.90 (s, 2H), 2.81 (d, J=19.0 Hz, 4H), 2.64 (d, J=17.0 Hz, 2H), 2.55 (d, J=9.9 Hz, 4H), 2.47 (s, 2H), 2.21 (s, 1H), 2.17-1.88 (m, 5H), 1.85-1.36 (m, 5H), 0.83 (d, J=5.5 Hz, 3H): [M+H]⁺=838.7.

### Example 244: (R,E)-N-((3S,4R)-1-(4-((R)-2,6-dioxopiperidin-3-yl)-3,5-difluorophenethyl)-3-methoxypiperidin-4-yl)-1¹,2⁶,7-trimethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁶-carboxamide

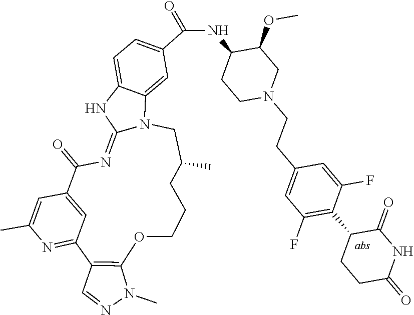 [SMILES: CO[C@H]1CN(CCc2cc(F)c([C@H]3CCC(=O)NC3=O)c(F)c2)CC[C@H]1NC(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.89 (s, 1H), 10.94 (s, 1H), 8.44 (s, 1H), 8.10 (s, 1H), 8.03 (d, J=7.6 Hz, 1H), 7.93 (s, 1H), 7.81 (d, J=8.4 Hz, 1H), 7.59-7.54 (m, 2H), 7.08 (d, J=10.2 Hz, 2H), 4.37 (d, J=4.7 Hz, 1H), 4.21 (t, J=11.7 Hz, 2H), 4.08-3.98 (m, 4H), 3.74 (s, 3H), 3.27 (s, 3H), 3.17 (s, 1H), 2.82 (d, J=12.4 Hz, 2H), 2.77 (d, J=7.1 Hz, 2H), 2.60 (d, J=6.3 Hz, 3H), 2.56 (s, 3H), 2.54 (s, 1H), 2.21-2.10 (m, 4H), 2.00 (d, J=10.8 Hz, 3H), 1.93 (s, 1H), 1.58-1.50 (m, 2H), 0.84 (d, J=6.4 Hz, 3H); [M+H]⁺=838.6.

### Example 245: (R,E)-N-((3R,4S)-1-(4-((R)-2,6-dioxopiperidin-3-yl)-3,5-difluorophenethyl)-3-methoxypiperidin-4-yl)-1¹,2⁵,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

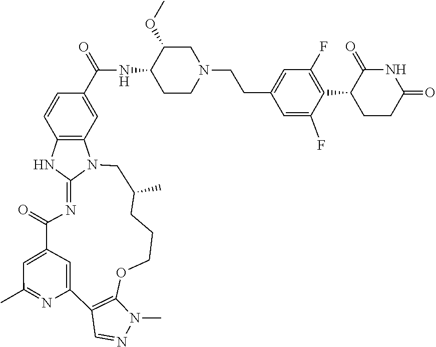 [SMILES: CO[C@@H]1CN(CCc2cc(F)c([C@H]3CCC(=O)NC3=O)c(F)c2)CC[C@@H]1NC(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.89 (s, 1H), 10.94 (s, 1H), 8.44 (s, 1H), 8.16 (s, 1H), 8.04 (d, J=7.4 Hz, 1H), 7.93 (s, 1H), 7.82 (d, J=8.4 Hz, 1H), 7.60-7.52 (m, 2H), 7.08 (d, J=10.3 Hz, 2H), 4.37 (d, J=3.9 Hz, 1H), 4.28-4.14 (m, 2H), 4.12-3.91 (m, 3H), 3.74 (s, 3H), 3.50 (s, 1H), 3.24-3.21 (m, 3H), 3.19-3.15 (m, 1H), 2.83-2.81 (m, 5H), 2.66-2.53 (m, 6H), 2.30-2.08 (m, 4H), 2.07-1.96 (m, 3H), 1.93-1.91 (m, 1H), 1.63-1.54 (m, 1H), 1.54-1.41 (m, 1H), 0.83 (d, J=6.4 Hz, 3H); [M+H]⁺=838.5.

### Example 246: (R,E)-N-((3S,4R)-1-(4-((R)-2,6-dioxopiperidin-3-yl)-3,5-difluorophenethyl)-3-fluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

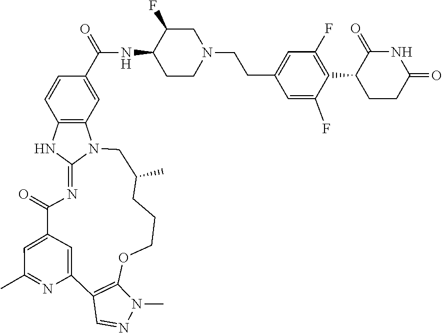 [SMILES: Cc1cc2cc(n1)-c1cnn(C)c1OCCC[C@@H](C)CN1/C(=N/C2=O)Nc2ccc(C(=O)N[C@@H]3CCN(CCc4cc(F)c([C@H]5CCC(=O)NC5=O)c(F)c4)C[C@@H]3F)cc21]

The titled compound was prepared in a manner similar to that in Example 260.

¹H NMR (500 MHz, DMSO) δ 12.91 (s, 1H), 10.95 (s, 1H), 8.44 (s, 1H), 8.36 (d, J=4.5 Hz, 1H), 8.16 (d, J=16.2 Hz, 1H), 7.93 (s, 1H), 7.84 (d, J=4.9 Hz, 1H), 7.61-7.45 (m, 2H), 7.06 (d, J=10.2 Hz, 2H), 4.81-4.75 (m, 1H), 4.37 (d, J=4.3 Hz, 1H), 4.21-4.18 (m, 2H), 4.04-4.01 (m, 3H), 3.74 (s, 3H), 3.28 (d, J=13.9 Hz, 1H), 3.02-2.98 (m, 1H), 2.86-2.83 (m, 1H), 2.83-2.73 (m, 3H), 2.61-2.58 (m, 2H), 2.55-2.52 (m, 4H), 2.21-2.16 (m, 4H), 2.07-1.97 (m, 3H), 1.96-1.87 (m, 1H), 1.74-1.62 (m, 1H), 1.54-1.42 (m, 1H), 0.90-0.78 (m, 3H); [M+H]⁺=826.7.

### Example 247: (R,E)-N-((3S,4S)-1-(4-((R)-2,6-dioxopiperidin-3-yl)-3,5-difluorophenethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

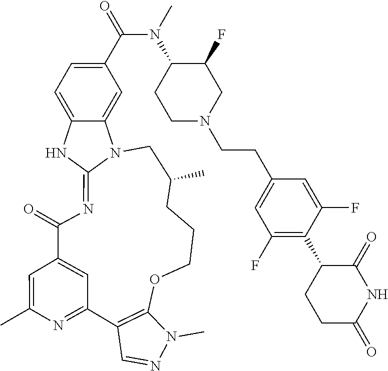 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)C[C@@H]3F)cc21]

The titled compound was prepared in a manner similar to that in Example 251.

¹H NMR (500 MHz, DMSO) δ 12.69 (s, 1H), 11.04-10.79 (m, 1H), 8.43 (s, 1H), 7.93 (s, 1H), 7.65 (s, 1H), 7.58 (t, J=3.8 Hz, 2H), 7.20 (s, 1H), 7.10-6.90 (m, 2H), 4.82-4.44 (m, 1H), 4.36 (d, J=4.1 Hz, 1H), 4.19 (d, J=11.8 Hz, 2H), 4.00 (s, 2H), 3.73 (s, 3H), 3.43 (d, J=21.0 Hz, 2H), 3.26-3.19 (m, 1H), 3.00-2.61 (m, 9H), 2.56 (s, 3H), 2.54 (s, 1H), 2.26-1.69 (m, 9H), 1.44 (s, 1H), 0.83 (d, J=6.2 Hz, 3H). [M+H]⁺=840.7.

### Example 250: (R,E)-N—((R)-1-(4-((R)-2,6-dioxopiperidin-3-yl)-3,5-difluorophenethyl)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

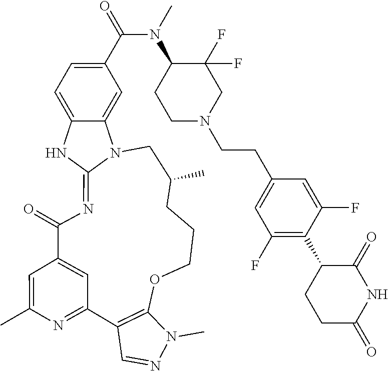 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)CC3(F)F)cc21]

The titled compound was prepared in a manner similar to that in Example 251.

¹H NMR (500 MHz, DMSO) δ 12.88 (s, 1H), 10.94 (s, 1H), 8.43 (s, 1H), 7.93 (s, 1H), 7.67 (s, 1H), 7.58 (d, J=7.3 Hz, 2H), 7.23 (s, 1H), 7.02 (s, 2H), 4.36 (d, J=4.5 Hz, 1H), 4.19 (d, J=11.2 Hz, 2H), 4.10-3.94 (m, 2H), 3.73 (s, 3H), 3.25-2.87 (m, 6H), 2.84-2.63 (m, 5H), 2.60-2.52 (m, 5H), 2.28-1.77 (m, 9H), 1.45 (d, J=6.7 Hz, 1H), 0.81 (d, J=6.1 Hz, 3H). [M+H]⁺=858.7.

### Example 251: (R,E)-N—((S)-1-(4-((R)-2,6-dioxopiperidin-3-yl)-3,5-difluorophenethyl)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

### Step 1: Tert-butyl (S)-4-(((benzyloxy)carbonyl)amino)-3,3-difluoropiperidine-1-carboxylate

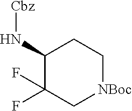 [SMILES: CC(C)(C)OC(=O)N1CC[C@H](NC(=O)OCc2ccccc2)C(F)(F)C1]

To a solution of tert-butyl (S)-4-amino-3,3-difluoropiperidine-1-carboxylate (5.3 g, 22.46 mmol) in DCM (80 mL) and DIEA (5.8 g, 44.92 mmol) was added CbzCl (4.96 g, 29.2 mmol) at 5-10° C. The reaction mixture was stirred at r.t for 3 hrs. The reaction was quenched with water (40 mL), and the resulting mixture was extracted with DCM (40 mL×3). The combined organic phase was washed with brine (20 mL), dried over Na₂SO₄, filtered and concentrated in vacuum. The residue was purified by silica gel column (PE:EA=2:1) to afford tert-butyl (S)-4-(((benzyloxy)carbonyl)amino)-3,3-difluoropiperidine-1-carboxylate (7.8 g, 93.6%). [M+H]⁺=371.2.

### Step 2: Tert-butyl (S)-4-(((benzyloxy)carbonyl)(methyl)amino)-3,3-difluoropiperidine-1-carboxylate

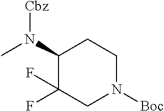 [SMILES: CN(C(=O)OCc1ccccc1)[C@H]1CCN(C(=O)OC(C)(C)C)CC1(F)F]

To a solution of tert-butyl (S)-4-(((benzyloxy)carbonyl)amino)-3,3-difluoropiperidine-1-carboxylate (7.8 g, 21.02 mmol) and iodomethane (5.9 g, 42.0 mmol) in THF (80 mL) was added NaH (1.26 g, 31.53 mmol, 60%). The reaction was stirred at r.t for overnight. The reaction was quenched with sat. NH₄Cl solution (50 mL) at 0-10° C., and the resulting mixture was extracted with EA (60 mL×3). The combined organic phase was washed with brine (20 mL), dried over Na₂SO₄, filtered and concentrated in vacuum. The residue was purified by silica gel column (PE:EA=3:1) to afford tert-butyl (S)-4-(((benzyloxy)carbonyl)(methyl)amino)-3,3-difluoropiperidine-1-carboxylate (7.2 g, 89%). [M+H]⁺=385.3.

### Step 3: Tert-butyl (S)-3,3-difluoro-4-(methylamino)piperidine-1-carboxylate

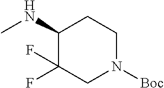 [SMILES: CN[C@H]1CCN(C(=O)OC(C)(C)C)CC1(F)F]

To a solution of tert-butyl (S)-4-(((benzyloxy)carbonyl)(methyl)amino)-3,3-difluoropiperidine-1-carboxylate (7.2 g, 18.7 mmol) in THF (100 mL) was added Pd/C (2.5 g, 10%). The mixture was stirred at r.t for 8 hr under hydrogen atmosphere. The reaction mixture was filtered through celite, and the filtrate was concentrated in vacuo to afford tert-butyl (S)-3,3-difluoro-4-(methylamino)piperidine-1-carboxylate (4.3 g, 91.7%). [M+H]⁺=251.1.

### Step 4: tert-butyl (S)-3,3-difluoro-4-((R,E)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate

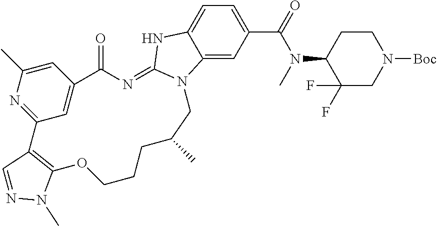 [SMILES: Cc1cc2cc(n1)-c1cnn(C)c1OCCC[C@@H](C)CN1/C(=N/C2=O)Nc2ccc(C(=O)N(C)[C@H]3CCN(C(=O)OC(C)(C)C)CC3(F)F)cc21]

To a mixture of (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxylic acid (2 g, 4.22 mmol) and tert-butyl (S)-3,3-difluoro-4-(methylamino)piperidine-1-carboxylate (1.37 g, 5.49 mmol) in CH₃CN (30 mL) was added TCFH (1.77 g, 6.33 mmol) and 1-methylimidazole (1.73 g, 21.1 mmol). The reaction was stirred at r.t. for 2 hrs. The reaction was quenched with H₂O (20 mL) and the resulting mixture was extracted with DCM (30 mL×3). The combined organic phase was washed with brine (30 mL), dried over Na₂SO₄, filtered and concentrated in vacuum. The residue was purified by silica gel column (DCM:CH₃OH=20:1) to afford the product (2.7 g, 90.6%). [M+H]⁺=707.2.

### Step 5: (R,E)-N—((S)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

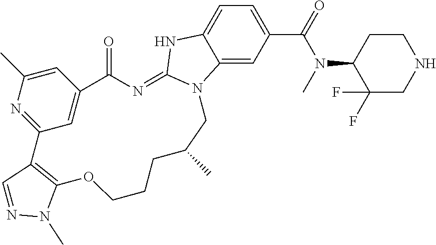 [SMILES: Cc1cc2cc(n1)-c1cnn(C)c1OCCC[C@@H](C)CN1/C(=N/C2=O)Nc2ccc(C(=O)N(C)[C@H]3CCNCC3(F)F)cc21]

To a solution of tert-butyl (S)-3,3-difluoro-4-((R,E)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate (2.7 g, 3.82 mmol) in DCM (30 mL) was added TFA (6 mL). The reaction was stirred at r.t. for 2 hrs. The reaction was concentrated in vacuo, the residue was dissolved in DCM (80 mL) and then washed with sat. aq. NaHCO₃ solution (30 mL) and brine (30 mL), dried over Na₂SO₄, filtered and concentrated in vacuum to afford the desired product (2.1 g, 90.5%). [M+H]⁺=607.2.

### Step 6: (R,E)-N—((S)-1-(4-((R)-2,6-dioxopiperidin-3-yl)-3,5-difluorophenethyl)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

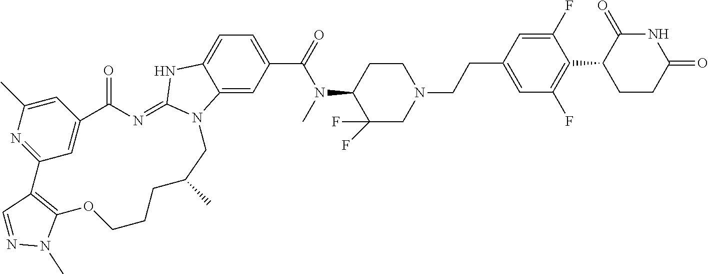 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)CC3(F)F)cc21]

To a mixture of (R,E)-N—((S)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide (2.1 g, 3.46 mmol) and (R)-2-(4-(2,6-dioxopiperidin-3-yl)-3,5-difluorophenyl)acetaldehyde (1.2 g, 4.5 mmol) in DCE (40 mL) was added STAB (1.83 g, 8.65 mmol). The reaction was stirred at 50° C. for 2 hrs. After cooling to r.t., the reaction was quenched with H₂O (20 mL), and the resulting mixture was extracted with DCM (30 mL×3). The combined organic phase was washed with brine (30 mL), dried over Na₂SO₄, filtered and concentrated in vacuum. The residue was purified by silica gel column (DCM:CH₃OH=20:1) to afford the desired product (1.3 g, 43.8%). ¹H NMR (500 MHz, DMSO) δ 12.88 (s, 1H), 10.94 (s, 1H), 8.43 (s, 1H), 7.93 (s, 1H), 7.69 (s, 1H), 7.59 (d, J=8.6 Hz, 2H), 7.24 (s, 1H), 7.01 (d, J=13.6 Hz, 2H), 4.36 (d, J=4.0 Hz, 1H), 4.20 (d, J=11.3 Hz, 2H), 4.00 (s, 2H), 3.73 (s, 3H), 2.99 (s, 5H), 2.78 (d, J=11.0 Hz, 5H), 2.56 (s, 4H), 2.53 (s, 2H), 2.30-2.06 (m, 4H), 1.97 (d, J=13.7 Hz, 5H), 1.45 (s, 1H), 0.84 (d, J=5.2 Hz, 3H). [M+H]⁺=858.6.

### Example 252: (R,E)-N—((3R,4S)-1-(4-((R)-2,6-dioxopiperidin-3-yl)-3,5-difluorophenethyl)-3-methoxypiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4) -pyridina-1(4,5)-pyrazolacycloundecaphane-5⁴-carboxamide

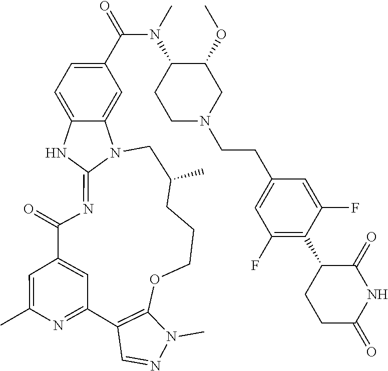 [SMILES: CO[C@@H]1CN(CCc2cc(F)c([C@H]3CCC(=O)NC3=O)c(F)c2)CC[C@@H]1N(C)C(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.86 (s, 1H), 10.94 (s, 1H), 8.43 (s, 1H), 7.93 (s, 1H), 7.68 (s, 1H), 7.57 (d, J=5.4 Hz, 2H), 7.24 (s, 1H), 7.06 (s, 2H), 4.36 (d, J=4.6 Hz, 1H), 4.20 (d, J=11.4 Hz, 2H), 4.01 (s, 2H), 3.73 (s, 3H), 3.30-3.28 (m, 4H), 2.99-2.96 (m, 4H), 2.82-2.79 (m, 4H), 2.57-2.55 (m, 7H), 2.27-2.07 (m, 4H), 1.97-1.94 (m, 4H), 1.54-1.49 (m, 3H), 0.82 (d, J=6.1 Hz, 3H); [M+H]⁺=852.4.

### Example 253: (R,E)-N—((3R,4R)-1-(4-((R)-2,6-dioxopiperidin-3-yl)-3,5-difluorophenethyl)-3-methoxypiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

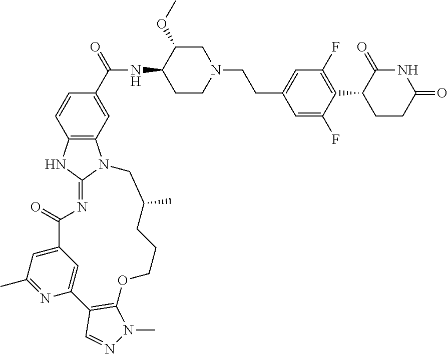 [SMILES: CO[C@@H]1CN(CCc2cc(F)c([C@H]3CCC(=O)NC3=O)c(F)c2)CC[C@H]1NC(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.89 (s, 1H), 10.95 (s, 1H), 8.44 (s, 1H), 8.25 (d, J=8.4 Hz, 1H), 8.05 (s, 1H), 7.92 (d, J=3.6 Hz, 1H), 7.78 (d, J=8.0 Hz, 1H), 7.56 (d, J=8.3 Hz, 2H), 7.05 (d. J=10.1 Hz, 2H), 4.34-4.32 (m, 1H), 4.24-4.21 (m, 2H), 3.98-3.95 (m, 2H), 3.80-3.77 (m, 1H), 3.74-3.71 (m, 3H), 2.92-2.89 (m, 2H), 2.81-2.78 (m, 5H), 2.69-2.60 (m, 4H), 2.58 (s, 5H), 2.26-2.07 (m, 2H), 2.07-1.96 (m, 3H), 1.91-1.87 (m, 1H), 1.80-1.76 (m, 2H), 1.62-1.44 (m, 2H), 0.84-0.81 (m, 3H), [M+H]⁺=838.7.

### Example 254: (R,E)-N—((3R,4R)-1-(4-((R)-2,6-dioxopiperidin-3-yl)-3,5-difluorophenethyl)-3-methoxypiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4) -pyridina-1(4,5)-pyrazolacycloundecaphane-5⁶-carboxamide

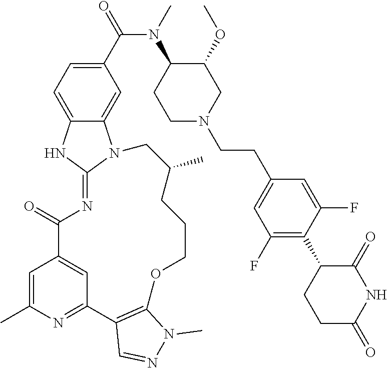 [SMILES: CO[C@@H]1CN(CCc2cc(F)c([C@H]3CCC(=O)NC3=O)c(F)c2)CC[C@H]1N(C)C(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.86 (s, 1H), 10.94 (s, 1H), 8.43 (s, 1H), 7.93 (s, 1H), 7.68 (s, 1H), 7.57 (d, J=5.4 Hz, 2H), 7.24 (s, 1H), 7.06 (s, 2H), 4.36 (d, J=4.6 Hz, 1H), 4.20 (d, J=11.4 Hz, 2H), 4.01 (s, 2H), 3.73 (s, 3H), 3.30-3.28 (m, 4H), 2.99-2.96 (m, 4H), 2.82-2.79 (m, 4H), 2.57-2.55 (m, 7H), 2.27-2.07 (m, 4H), 1.97-1.94 (m, 4H), 1.54-1.49 (m, 3H), 0.82 (d, J=6.1 Hz, 3H), [M+H]⁺=852.4.

### Example 255: (R,E)-N—((3S,4R)-1-(4-((R)-2,6-dioxopiperidin-3-yl)-3,5-difluorophenethyl)-3-methoxypiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4) -pyridina-1(4,5)-pyrazolacycloundecaphane-5⁶-carboxamide

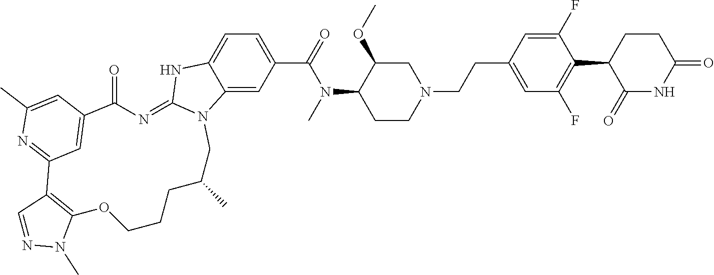 [SMILES: CO[C@H]1CN(CCc2cc(F)c([C@H]3CCC(=O)NC3=O)c(F)c2)CC[C@H]1N(C)C(=O)c1ccc2c(c1)N1C[C@H](C)CCCOc3c(cnn3C)-c3cc(cc(C)n3)C(=O)/N=C/1N2]

The titled compound was prepared in a manner similar to that in Example 260.

¹H NMR (500 MHz, DMSO) δ 12.85 (s, 1H), 10.93 (s, 1H), 8.43 (s, 1H), 7.93 (s, 1H), 7.68 (s, 1H), 7.57 (d, J=7.7 Hz, 2H), 7.23 (s, 1H), 7.06 (s, 2H), 4.36 (d, J=4.4 Hz, 2H), 4.19 (d, J=11.4 Hz, 2H), 4.11-3.93 (m, 2H), 3.73 (s, 4H), 3.28 (s, 4H), 2.96 (s, 4H), 2.78 (d, J=10.6 Hz, 3H), 2.62 (d, J=18.0 Hz, 1H), 2.55 (d, J=10.0 Hz, 5H), 2.36 (s, 1H), 2.29-1.84 (m, 7H), 1.83-1.28 (m, 3H), 0.82 (d, J=6.4 Hz, 3H).: [M+H]⁺=852.6.

### Example 257: (R,E)-N—((3R,4R)-1-(4-((R)-2,6-dioxopiperidin-3-yl)-3,5-difluorophenethyl)-3-methylpiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

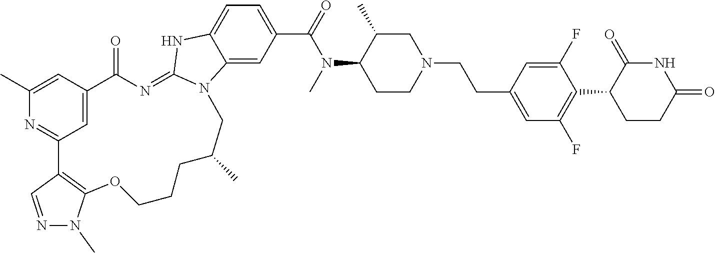 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3C)cc21]

The titled compound was prepared in a manner similar to that in Example 260. ¹H NMR (500 MHz, DMSO) δ_{H} 12.87 (s, 1H), 10.94 (d, J=11.4 Hz, 1H), 8.43 (s, 1H), 7.93 (s, 1H), 7.68-7.50 (m, 3H), 7.16 (d, J=7.9 Hz, 1H), 7.08-6.93 (m, 2H), 4.36 (s, 1H), 4.22-3.96 (m, 5H), 3.73 (s, 3H), 3.15-2.65 (m, 1H), 2.55 (d, J=10.3 Hz, 4H), 2.27-1.58 (m, 10H), 1.45 (s, 1H), 0.93-0.64 (m, 6H). [M+H]⁺=836.6.

### Example 258: (R,E)-N—((2S,4R)-1-(4-((R)-2,6-dioxopiperidin-3-yl)-3,5-difluorophenethyl)-2-methylpiperidin-4-yl) -N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

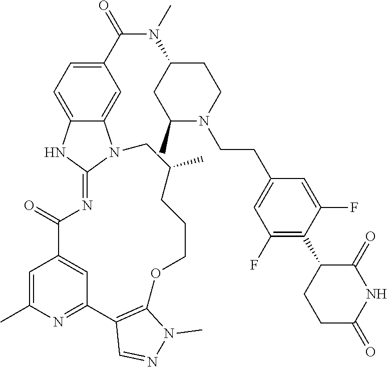 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)[C@@H](C)C3)cc21]

The titled compound was prepared in a manner similar to that in Example 260.

¹H NMR (500 MHz, DMSO) δ 12.85 (s, 1H), 10.93 (s, 1H), 8.43 (s, 1H), 7.92 (s, 1H), 7.66 (s, 1H), 7.56 (d, J=9.9 Hz, 2H), 7.21 (s, 1H), 7.00 (s, 2H), 4.36 (d, J=4.5 Hz, 1H), 4.19 (d, J=11.3 Hz, 2H), 4.08-3.95 (m, 2H), 3.73 (s, 3H), 3.32 (s, 2H), 2.90-2.76 (m, 6H), 2.64 (s, 4H), 2.56 (s, 3H), 2.17-2.04 (m, 2H), 2.01-1.92 (m, 4H), 1.91 (s, 2H), 1.76 (d, J=8.8 Hz, 2H), 1.50-1.47 (m, 4H), 1.06 (s, 1H), 0.82 (d, J=6.3 Hz, 3H); [M+H]⁺=836.4.

### Example 259: (R,E)-N—((2R,4S)-1-(4-((R)-2,6-dioxopiperidin-3-yl)-3,5-difluorophenethyl)-2-methylpiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

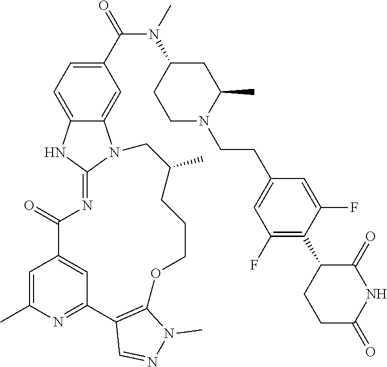 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)[C@H](C)C3)cc21]

The titled compound was prepared in a manner similar to that in Example 260.

¹H NMR (500 MHz, DMSO) δ 12.87 (s, 1H), 10.94 (s, 1H), 8.43 (s, 1H), 7.93 (s, 1H), 7.68 (s, 1H), 7.57 (s, 2H), 7.23 (s, 1H), 7.11 (s, 1H), 6.99 (s, 1H), 4.36 (d, J=4.7 Hz, 1H), 4.19 (s, 2H), 4.00 (s, 2H), 3.73 (s, 3H), 3.29 (s, 4H), 2.92-2.71 (m, 6H), 2.69-2.52 (m, 7H), 2.36 (s, 2H), 2.28-2.12 (m, 3H), 2.02-1.86 (m, 4H), 1.76 (s, 1H), 1.60 (s, 1H), 1.44 (s, 2H), 0.82 (d, J=6.3 Hz, 3H). [M+H]⁺=836.7

### Example 260: (R,E)-N—((R)-1-(4-((R)-2,6-dioxopiperidin-3-yl)-3,5-difluorophenethyl)-3,3-difluoropiperidin-4-yl)-1,2⁶,7-trimethyl-3-oxo-5²,5³-dihydro-1¹H,5′ H-11-oxa-4-aza-5(2,1)-benzo[d]imidazola-2(2,4)-pyridina-1(4,5)-pyrazolacycloundecaphane-5⁶-carboxamide

### Step 1 tert-butyl (R)-3,3-difluoro-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate

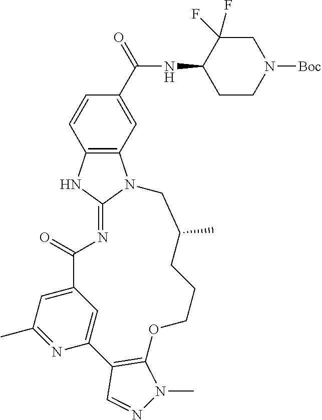 [SMILES: Cc1cc2cc(n1)-c1cnn(C)c1OCCC[C@@H](C)CN1/C(=N/C2=O)Nc2ccc(C(=O)N[C@@H]3CCN(C(=O)OC(C)(C)C)CC3(F)F)cc21]

A mixture of (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxylic acid (800 mg, 1.68 mmol), tert-butyl (R)-4-amino-3,3-difluoropiperidine-1-carboxylate (477 mg, 2.0 mmol), T₃P (1.6 g, 2.52 mmol) and DIEA (650 mg, 5.0 mmol) in DCM (20 mL) was stirred in a round bottom flask at 25° C. for 3 hours. The mixture was evaporated in vacuum to afford the crude product, which was purified with silica gel column chromatography (PE:EA=100:0˜2:1 gradient elution) to give the title product (900 mg, 78%). [M+H]⁺=693.4.

### Step 2: (R,E)-N—((R)-3,3-difluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

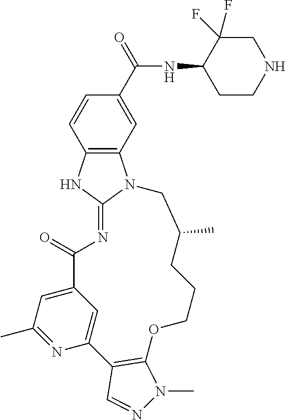 [SMILES: Cc1cc2cc(n1)-c1cnn(C)c1OCCC[C@@H](C)CN1/C(=N/C2=O)Nc2ccc(C(=O)N[C@@H]3CCNCC3(F)F)cc21]

A solution of tert-butyl (R)-3,3-difluoro-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate (695 mg, 1.0 mmol) in TFA/DCM=1/4 (5 mL) was stirred in a round bottom flask at room temperature for 2 h. The mixture was evaporated in vacuum to afford the crude product (590 mg, 99%), which was used for next step without further purification. [M+H]⁺=593.2.

### Step 3: (R,E)-N—((R)-1-(4-((R)-2,6-dioxopiperidin-3-yl)-3,5-difluorophenethyl)-3,3-difluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

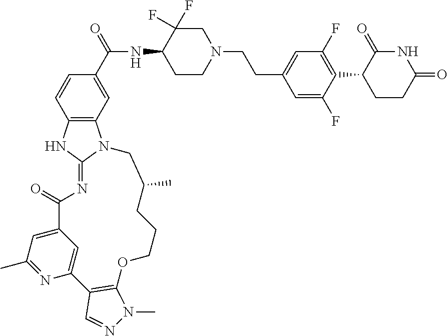 [SMILES: Cc1cc2cc(n1)-c1cnn(C)c1OCCC[C@@H](C)CN1/C(=N/C2=O)Nc2ccc(C(=O)N[C@@H]3CCN(CCc4cc(F)c([C@H]5CCC(=O)NC5=O)c(F)c4)CC3(F)F)cc21]

A mixture of (R,E)-N—((R)-3,3-difluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide (592 mg, 1 mmol) and (R)-2-(4-(2,6-dioxopiperidin-3-yl)-3,5-difluorophenyl)acetaldehyde (296 mg, 1.1 mmol) in 1,2-dichloromethane (15 mL)) was stirred in a round bottom flask at room temperature for 1 hour. To the mixture was added NaBH(OAc)₃ (464 mg, 2.2 mmol) and the reaction was stirred in a round bottom flask at room temperature for 2 hrs. Then the mixture was evaporated in vacuum to afford the crude product, which was purified with silica gel column chromatography (DCM:MeOH=100:0˜ 80:20 gradient elution) to give the product (420 mg, 50%). ¹H NMR (500 MHz, DMSO) δ 12.92 (s, 1H), 10.95 (s, 1H), 8.43 (d, J=8.2 Hz, 2H), 8.14 (s, 1H), 7.93 (s, 1H), 7.85 (d, J=8.4 Hz, 1H), 7.57 (d, J=7.9 Hz, 2H), 7.06 (d, J=10.1 Hz, 2H), 4.46 (s, 1H), 4.37 (d, J=4.2 Hz, 1H), 4.27-4.15 (m, 2H), 4.05-3.93 (m, 2H), 3.74 (s, 3H), 3.27 (s, 1H), 3.01-2.97 (m, 1H), 2.86-2.84 (m, 1H), 2.80-2.75 (m, 3H), 2.73-2.65 (m, 2H), 2.60-2.54 (m, 4H), 2.49-2.42 (m, 1H), 2.38-2.06 (m, 3H), 2.06-1.97 (m, 2H), 1.92-1.89 (m, 2H), 1.85-1.78 (m, 1H), 1.54-1.43 (m, 1H), 0.84 (d, J=6.4 Hz, 3H), [M+H]⁺=844.7.

### Example 261: (R,E)-N—((S)-1-(4-((R)-2,6-dioxopiperidin-3-yl)-3,5-difluorophenethyl)-3,3-dimethylpiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

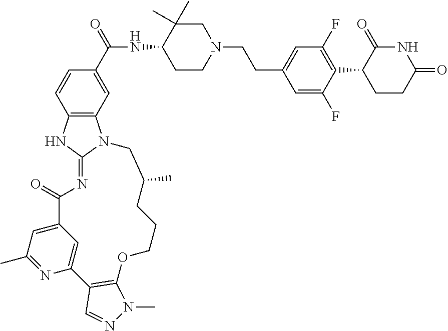 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)CC3(C)C)cc21]

The titled compound was prepared in a manner similar to that in Example 260.

¹H NMR (500 MHz, DMSO) δ 12.88 (s, 1H), 10.94 (s, 1H), 8.44 (s, 1H), 8.04 (s, 1H), 7.87 (dd, J=21, 8 Hz, 3H), 7.58 (s, 2H), 7.05 (d, J=6.2 Hz, 2H), 4.36 (s, 1H), 4.21 (s, 2H), 4.01 (s, 2H), 3.88-3.63 (m, 4H), 3.06-2.71 (m, 5H), 2.55-2.51 (m, 7H), 2.33-1.75 (m, 8H), 1.65-1.41 (m, 2H), 1.09-0.93 (m, 3H), 0.92-0.75 (m, 6H); [M+H]⁺=836.7.

### Example 262: (R,E)-N—((R)-1-(4-((R)-2,6-dioxopiperidin-3-yl)-3,5-difluorophenethyl)-3,3-dimethylpiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

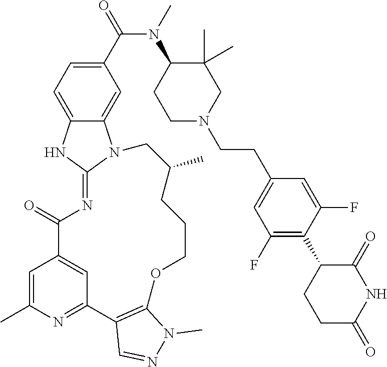 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)CC3(C)C)cc21]

The titled compound was prepared in a manner similar to that in Example 260.

¹H NMR (500 MHz, DMSO) δ 12.85 (s, 1H), 10.93 (s, 1H), 8.43 (s, 1H), 7.92 (s, 1H), 7.66 (s, 1H), 7.56 (d, J=9.9 Hz, 2H), 7.21 (s, 1H), 7.00 (s, 2H), 4.36 (d, J=4.5 Hz, 1H), 4.19 (d, J=11.3 Hz, 2H), 4.08-3.95 (m, 2H), 3.73 (s, 3H), 3.32 (s, 2H), 2.90-2.76 (m, 4H), 2.64 (s, 4H), 2.56 (s, 3H), 2.17-2.04 (m, 2H), 2.01-1.92 (m, 4H), 1.91 (s, 2H), 1.76 (d, J=8.8 Hz, 2H), 1.50-1.47 (m, 3H), 1.06 (s, 6H), 0.82 (d, J=6.3 Hz, 3H); [M+H]⁺=850.6.

### Example 263: (R,E)-N—((R)-1-(4-((R)-2,6-dioxopiperidin-3-yl)-3,5-difluorophenethyl)-3,3-dimethylpiperidin-4-yl)-1¹,2⁶,7-trimethyl-3-oxo-5²,5³-dihydro-1H,5¹H-11-oxa-4-aza-5(2,1)-benzo[d]imidazola-2(2,4)-pyridina-1(4,5)-pyrazolacycloundecaphane-5⁶-carboxamide

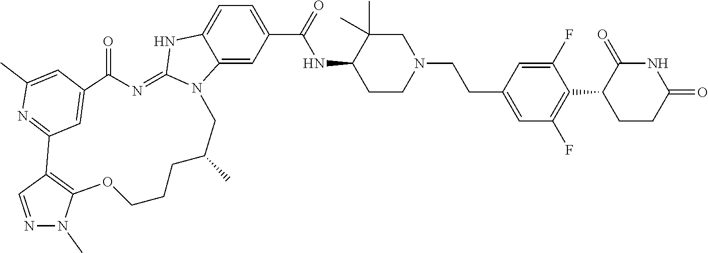 [SMILES: Cc1cc2cc(n1)-c1cnn(C)c1OCCC[C@@H](C)CN1/C(=N/C2=O)Nc2ccc(C(=O)N[C@@H]3CCN(CCc4cc(F)c([C@H]5CCC(=O)NC5=O)c(F)c4)CC3(C)C)cc21]

The titled compound was prepared in a manner similar to that in Example 260.

¹H NMR (500 MHz, DMSO) δ 12.88 (s, 1H), 10.95 (s, 1H), 8.44 (s, 1H), 8.05 (s, 1H), 7.93 (s, 1H), 7.87 (d, J=9.1 Hz, 1H), 7.79 (d, J=8.4 Hz, 1H), 7.63-7.47 (m, 2H), 7.05 (d, J=10.2 Hz, 2H), 4.37 (d, J=4.3 Hz, 1H), 4.30-4.15 (m, 2H), 4.01 (d, J=9.2 Hz, 2H), 3.84 (s, 1H), 3.74 (s, 3H), 2.96 (d, J=9.6 Hz, 1H), 2.87-2.69 (m, 4H), 2.62 (d, J=11.2 Hz, 1H), 2.56 (s, 5H), 2.48-2.38 (m, 1H), 2.12 (d, J=3.3 Hz, 2H), 2.07-1.72 (m, 6H), 1.51 (d, J=12.4 Hz, 2H), 1.01 (s, 3H), 0.83 (d, J=6.5 Hz, 6H); [M+H]⁺=836.9.

### Example 264: (R,E)-N—((1R,3S,5S)-8-(4-((R)-2,6-dioxopiperidin-3-yl)-3,5-difluorophenethyl)-8-azabicyclo[3.2.1]octan-3-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

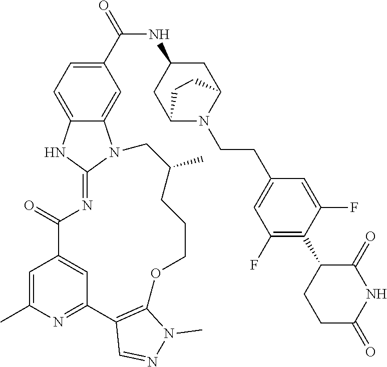 [SMILES: Cc1cc2cc(n1)-c1cnn(C)c1OCCC[C@@H](C)CN1/C(=N/C2=O)Nc2ccc(C(=O)N[C@@H]3C[C@H]4CC[C@@H](C3)N4CCc3cc(F)c([C@H]4CCC(=O)NC4=O)c(F)c3)cc21]

The titled compound was prepared in a manner similar to that in Example 260.

¹H NMR (500 MHz, DMSO) δ 12.89 (s, 1H), 10.95 (s, 1H), 8.43 (s, 1H), 7.96 (s, 1H), 7.93 (s, 1H), 7.89 (d, J=4.3 Hz, 1H), 7.68 (d, J=8.2 Hz, 1H), 7.58 (d, J=8.0 Hz, 2H), 7.05 (d, J=10.0 Hz, 2H), 4.36 (d, J=4.6 Hz, 1H), 4.27-4.17 (m, 2H), 4.05-3.94 (m, 3H), 3.74 (s, 3H), 3.26 (s, 2H), 2.89-2.72 (m, 4H), 2.56 (s, 3H), 2.54 (s, 3H), 2.28-1.88 (m, 1H), 1.81 (t, J=11.8 Hz, 2H), 1.48 (s, 1H), 0.84 (d, J=6.4 Hz, 3H). [M+H]⁺=834.7

### Example 265: (R,E)-N—((1R,3S,5S)-8-(4-((R)-2,6-dioxopiperidin-3-yl)-3,5-difluorophenethyl)-8-azabicyclo[3.2.1]octan-3-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

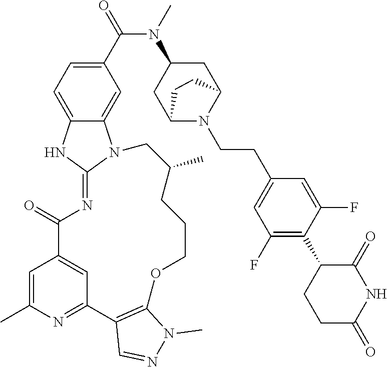 [SMILES: Cc1cc2cc(n1)-c1cnn(C)c1OCCC[C@@H](C)CN1/C(=N/C2=O)Nc2ccc(C(=O)N(C)[C@@H]3C[C@H]4CC[C@@H](C3)N4CCc3cc(F)c([C@H]4CCC(=O)NC4=O)c(F)c3)cc21]

The titled compound was prepared in a manner similar to that in Example 260.

¹H NMR (500 MHz, DMSO) δ 12.87 (s, 1H), 10.93 (s, 1H), 8.43 (s, 1H), 7.93 (s, 1H), 7.66 (s, 1H), 7.58 (s, 2H), 7.20 (s, 1H), 7.03 (s, 2H), 4.36 (d, J=4.3 Hz, 1H), 4.19 (d, J=12.8 Hz, 2H), 4.10-3.96 (m, 2H), 3.73 (s, 3H), 3.59 (s, 1H), 3.27-3.12 (m, 3H), 2.85-2.63 (m, 7H), 2.56 (s, 3H), 2.49-2.40 (m, 2H), 2.21 (s, 1H), 2.17-2.06 (m, 1H), 2.02-1.82 (m, 6H), 1.64 (s, 2H), 1.57-1.32 (m, 3H), 0.82 (d, J=6.4 Hz, 3H). [M+H]⁺=848.7

### Example 266: (7R,E)-N-(9-(4-((R)-2,6-dioxopiperidin-3-yl)-3,5-difluorophenethyl)-9-azabicyclo[3.3.1]nonan-3-yl) -N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

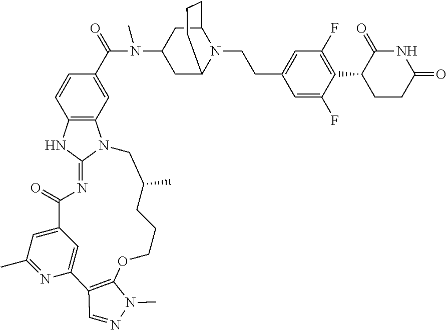 [SMILES: Cc1cc2cc(n1)-c1cnn(C)c1OCCC[C@@H](C)CN1/C(=N/C2=O)Nc2ccc(C(=O)N(C)C3CC4CCCC(C3)N4CCc3cc(F)c([C@H]4CCC(=O)NC4=O)c(F)c3)cc21]

The titled compound was prepared in a manner similar to that in Example 260.

¹H NMR (500 MHz, DMSO) δ 12.85 (s, 1H), 10.93 (s, 1H), 8.43 (s, 1H), 7.92 (d, J=3.2 Hz, 1H), 7.67 (s, 1H), 7.57 (s, 2H), 7.22 (s, 1H), 7.04 (s, 2H), 4.39-4.34 (m, 1H), 4.27-4.12 (m, 2H), 4.00-3.98 (, 2H), 3.73 (s, 3H), 3.01-2.89 (m, 3H), 2.87-2.74 (m, 8H), 2.72-2.64 (m, 6H), 2.29-2.16 (m, 2H), 2.15-2.03 (m, 3H), 2.01-1.89 (m, 3H), 1.75-1.58 (m, 2H), 1.57-1.34 (m, 3H), 1.26-1.01 (m, 2H), 0.81 (d, J=5.4 Hz, 3H):[M+H]⁺=862.7.

### Example 267: (R,E)-N—((2S,4S)-1-(4-((R)-2,6-dioxopiperidin-3-yl)-3,5-difluorophenethyl)-2-methylpiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

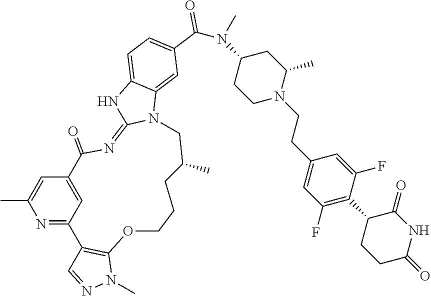 [SMILES: Cc1cc2cc(n1)-c1cnn(C)c1OCCC[C@@H](C)CN1/C(=N/C2=O)Nc2ccc(C(=O)N(C)[C@H]3CCN(CCc4cc(F)c([C@H]5CCC(=O)NC5=O)c(F)c4)[C@@H](C)C3)cc21]

The titled compound was prepared in a manner similar to that in Example 260. ¹H NMR (500 MHz, DMSO) δ 12.86 (s, 1H), 10.93 (s, 1H), 8.43 (s, 1H), 7.92 (s, 1H), 7.67 (s, 1H), 7.57 (d, J=7.3 Hz, 2H), 7.23 (s, 1H), 6.95 (s, 2H), 4.36 (d, J=4.4 Hz, 1H), 4.20 (d, J=11.8 Hz, 2H), 4.06-3.98 (m, 2H), 3.73 (s, 3H), 2.86-2.79 (m, 8H), 2.56 (s, 4H), 2.54 (s, 2H), 2.48-2.43 (m, 2H), 2.21 (s, 1H), 2.09 (s, 11H), 2.01-1.89 (m, 4H), 1.78 (s, 1H), 1.70 (s, 1H), 1.58-1.39 (m, 3H), 1.11-1.01 (m, 3H), 0.83 (d, J=6.2 Hz, 3H); [M+H]⁺=836.7.

### Example 268: (R,E)-N—((2R,4R)-1-(4-((R)-2,6-dioxopiperidin-3-yl)-3,5-difluorophenethyl)-2-methylpiperidin-4-yl) -N,1¹,2⁶,7-tetramethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁶-carboxamide

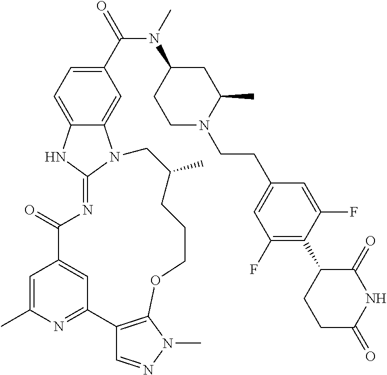 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)[C@H](C)C3)cc21]

The titled compound was prepared in a manner similar to that in Example 260. ¹H NMR (500 MHz, DMSO) δ 12.86 (s, 1H), 10.93 (s, 1H), 8.43 (s, 1H), 7.92 (s, 1H), 7.67 (s, 1H), 7.57 (d, J=6.7 Hz, 2H), 7.22 (s, 1H), 6.95 (s, 2H), 4.42-4.32 (m, 1H), 4.19 (t, J=9.4 Hz, 2H), 4.08-3.97 (m, 2H), 3.73 (s, 3H), 3.00-2.70 (m, 8H), 2.66-2.53 (m, 6H), 2.49-2.40 (m, 2H), 2.27-2.17 (m, 1H), 2.16-2.05 (m, 1H), 2.03-1.87 (m, 4H), 1.78 (d, J=11.6 Hz, 1H), 1.68 (d, J=11.3 Hz, 1H), 1.62-1.40 (m, 3H), 1.17-0.96 (m, 3H), 0.83 (d, J=6.1 Hz, 3H). [M+H]⁺=836.7

### Example 269: (R,E)-N—((2R,4R)-1-(4-((R)-2,6-dioxopiperidin-3-yl)-3,5-difluorophenethyl)-2-methylpiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

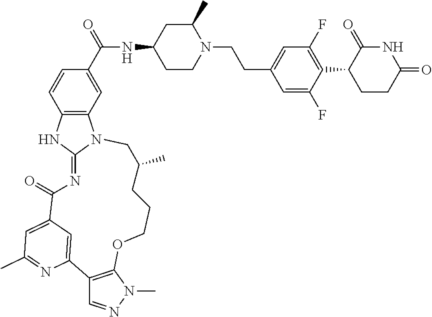 [SMILES: Cc1cc2cc(n1)-c1cnn(C)c1OCCC[C@@H](C)CN1/C(=N/C2=O)Nc2ccc(C(=O)N[C@@H]3CCN(CCc4cc(F)c([C@H]5CCC(=O)NC5=O)c(F)c4)[C@H](C)C3)cc21]

The titled compound was prepared in a manner similar to that in Example 260.

¹H NMR (500 MHz, DMSO) δ 12.89 (s, 1H), 10.95 (s, 1H), 8.44 (s, 1H), 8.18 (s, 1H), 8.06 (s, 1H), 7.93 (s, 1H), 7.78 (d, J=8.9 Hz, 1H), 7.57 (d, J=6.9 Hz, 1H), 7.54 (s, 1H), 7.04 (d, J=10.1 Hz, 2H), 4.36-4.34 (m, 1H), 4.22-4.20 (m, 2H), 3.98-3.95 (m, 2H), 3.88-3.85 (m, 1H), 3.74-3.71 (m, 3H), 3.03-3.01 (m, 1H), 2.94-2.91 (m, 1H), 2.89-2.79 (m, 2H), 2.76-2.74 (m, 2H), 2.55-2.53 (m, 5H), 2.42-2.29 (m, 2H), 2.22-2.20 (m, 1H), 2.13-2.11 (m, 1H), 2.01-1.98 (m, 2H), 1.93-1.90 (m, 1H), 1.85-1.82 (m, 2H), 1.64-1.43 (m, 2H), 1.33-1.31(m, 1H), 1.09 (s, 3H), 0.83 (d, J=6.5 Hz, 3H); [M+H]⁺=822.7.

### Example 270: (R,E)-N—((2S,4S)-1-(4-((R)-2,6-dioxopiperidin-3-yl)-3,5-difluorophenethyl)-2-methylpiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

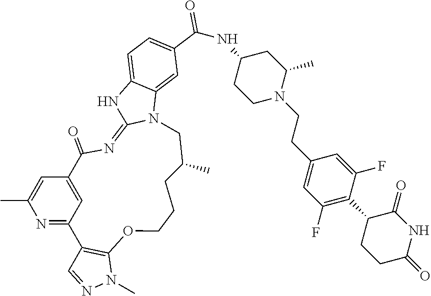 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)[C@@H](C)C3)cc21]

The titled compound was prepared in a manner similar to that in Example 260. ¹H NMR (50) MHz, DMSO) δ 12.88 (s, 1H), 10.95 (s, 1H), 8.44 (s, 1H), 8.19 (d, J=7.8 Hz, 1H), 8.05 (s, 1H), 7.93 (s, 1H), 7.80-7.75 (m, 1H), 7.59-7.52 (m, 2H), 7.03 (d, J=10.0 Hz, 2H), 4.37 (d, J=4.7 Hz, 1H), 4.23-4.18 (m, 2H), 4.03-3.93 (m, 2H), 3.89-3.83 (m, 1H), 3.74 (s, 3H), 3.04 (d, J=11.6 Hz, 1H), 2.97-2.91 (m, 1H), 2.85-2.81 (m, 2H), 2.74 (t, .=7.4 Hz, 2H), 2.56 (s, 5H), 2.36 (s, 1H), 2.29 (d, J=11.8 Hz, 1H), 2.22 (s, 1H), 2.16-2.10 (m, 1H), 2.04-1.98 (m, 2H), 1.93 (d, J=10.5 Hz, 1H), 1.83 (t, J=13.9 Hz, 2H), 1.61-1.54 (m, 1H), 1.49 (s, 1H), 1.34-1.27 (m, 1H), 1.07 (d, J=5.2 Hz, 3H), 0.84 (d, J=6.5 Hz, 3H); [M+H]⁺=822.5.

### Example 271: (R,E)-N—((R)-1-(1-(4-((R)-2,6-dioxopiperidin-3-yl)-3,5-difluorophenethyl)piperidin-4-yl)ethyl)-1¹,2⁶, 7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

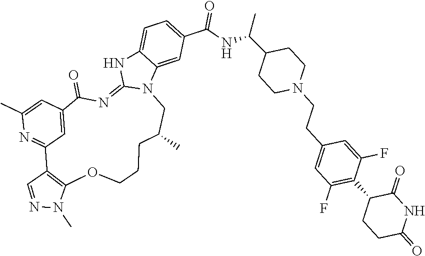 [SMILES: Cc1cc2cc(n1)-c1cnn(C)c1OCCC[C@@H](C)CN1/C(=N/C2=O)Nc2ccc(C(=O)N[C@H](C)C3CCN(CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.88 (s, 1H), 10.94 (s, 1H), 8.44 (s, 1H), 8.13 (d, J=8.6 Hz, 1H), 8.06 (s, 1H), 7.93 (s, 1H), 7.78 (d, J=8.3 Hz, 1H), 7.59-7.53 (m, 2H), 7.01 (d, J=10.0 Hz, 2H), 4.36 (d, J=4.5 Hz, 1H), 4.25-4.16 (m, 2H), 4.04-3.95 (m, 2H), 3.91 (dd, J=15.0, 7.4 Hz, 1H), 3.74 (s, 3H), 2.97 (s, 2H), 2.82 (dd, J=12.8, 4.8 Hz, 2H), 2.78-2.73 (m, 2H), 2.56 (s, 3H), 2.54 (d, J=7.0 Hz, 3H), 2.23 (s, 1H), 2.15-2.08 (m, 1H), 2.04-1.88 (m, 5H), 1.72 (d, J=11.4 Hz, 2H), 1.52-1.40 (m, 2H), 1.24 (s, 2H), 1.16 (d, J=6.7 Hz, 3H), 0.83 (d, J=6.5 Hz, 3H); [M+H]⁺=836.6.

### Example 272: (R,E)-N—((S)-1-(1-(4-((R)-2,6-dioxopiperidin-3-yl)-3,5-difluorophenethyl)piperidin-4-yl)ethyl)-N,1,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

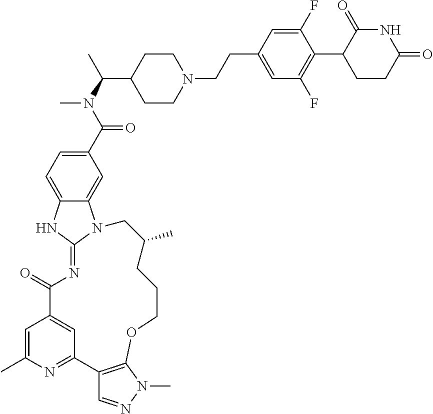 [SMILES: Cc1cc2cc(n1)-c1cnn(C)c1OCCC[C@@H](C)CN1/C(=N/C2=O)Nc2ccc(C(=O)N(C)[C@@H](C)C3CCN(CCc4cc(F)c(C5CCC(=O)NC5=O)c(F)c4)CC3)cc21]

The titled compound was prepared in a manner similar to that in Example 260. ¹H NMR (500 MHz, DMSO) δ 12.84 (d, J=15.2 Hz, 1H), 10.92 (s, 1H), 8.43 (s, 1H), 7.93 (d, J=5.4 Hz, 1H), 7.65 (d, J=15.7 Hz, 1H), 7.58 (s, 2H), 7.22 (s, 1H), 7.04 (s, 1H), 6.95 (s, 1H), 4.44-4.38 (m, 1H), 4.18 (s, 2H), 4.00 (s, 2H), 3.73 (s, 3H), 3.32-3.25 (m, 2H), 3.10-2.71 (m, 7H), 2.57-2.46 (m, 8H), 2.31-2.09 (m, 8H), 1.73 (s, 1H), 1.45 (s, 1H), 1.20 (m, 1H), 1.12-0.91 (m, 4H), 0.83 (s, 3H), 0.60 (s, 1H). [M+H]⁺=850.7

### Example 273: (R,E)-N—(((S)-1-(4-((R)-2,6-dioxopiperidin-3-yl)-3,5-difluorophenethyl)-3,3-difluoropiperidin-4-yl) methyl)-N,1′,2,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

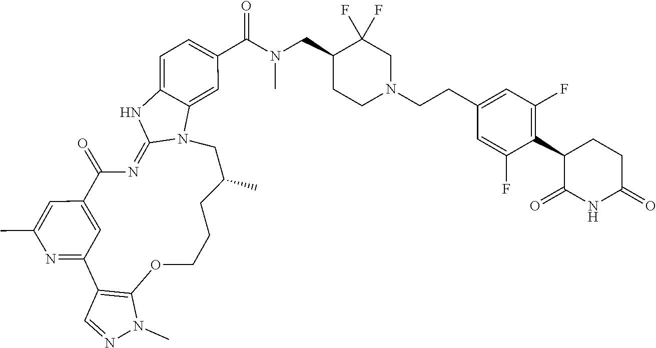 [SMILES: Cc1cc2cc(n1)-c1cnn(C)c1OCCC[C@@H](C)CN1/C(=N/C2=O)Nc2ccc(C(=O)N(C)C[C@@H]3CCN(CCc4cc(F)c([C@H]5CCC(=O)NC5=O)c(F)c4)CC3(F)F)cc21]

The titled compound was prepared in a manner similar to that in Example 260. ¹H NMR (500 MHz, DMSO) δ 12.86 (s, 1H), 10.93 (s, 1H), 8.43 (s, 1H), 7.92 (s, 1H), 7.67 (s, 1H), 7.57-7.56 (m, 2H), 7.25 (s, 1H), 7.04-6.97 (m, 2H), 4.40-4.31 (m, 1H), 4.21-4.19 (m, 2H), 4.04-4.00 (m, 2H), 3.73 (s, 3H), 3.59-3.50 (m, 1H), 3.03-2.91 (m, 4H), 2.85-2.73 (m, 5H), 2.69-2.59 (m, 5H), 2.56 (s, 3H), 2.43-2.34 (m, 1H), 2.24-2.06 (m, 4H), 2.03-1.82 (m, 4H), 1.55-1.40 (m, 1H), 0.81 (d, J=5.0 Hz, 3H); [M+H]⁺=872.7.

### Example 274: (R,E)-N—(((R)-1-(4-((R)-2,6-dioxopiperidin-3-yl)-3,5-difluorophenethyl)-3,3-difluoropiperidin-4-yl) methyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

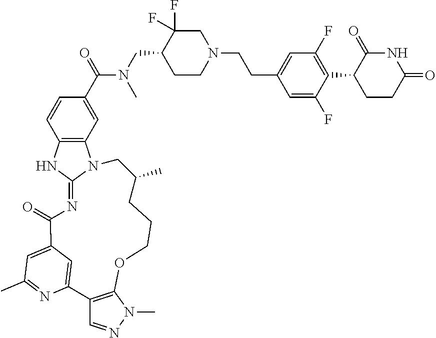 [SMILES: Cc1cc2cc(n1)-c1cnn(C)c1OCCC[C@@H](C)CN1/C(=N/C2=O)Nc2ccc(C(=O)N(C)C[C@H]3CCN(CCc4cc(F)c([C@H]5CCC(=O)NC5=O)c(F)c4)CC3(F)F)cc21]

The titled compound was prepared in a manner similar to that in Example 260. ¹H NMR (500 MHz, DMSO) δ 12.86 (s, 1H), 10.94 (s, 1H), 8.43 (s, 1H), 7.93 (s, 1H), 7.68 (s, 1H), 7.57 (d, J=8.0 Hz, 2H), 7.25 (s, 1H), 7.01-6.99 (d, 2H), 4.36-4.33 (m, 1H), 4.18-4.16 (m, 2H), 4.01-3.99 (m, 2H), 3.73 (s, 4H), 3.53-3.50 (m, 1H), 3.20 (s, 1H), 3.05-2.90 (m, 4H), 2.87-2.72 (m, 4H), 2.65-2.63 (m, 2H), 2.55-2.53 (m, 4H), 2.43-2.30 (m, 1H), 2.29-1.79 (m, 8H), 1.54-1.40 (m, 2H), 0.81 (d, J=6.4 Hz, 3H); [M+H]⁺=872.7.

### Example 275: (R,E)-N-(2-(1-(4-((R)-2,6-dioxopiperidin-3-yl)-3,5-difluorophenethyl)piperidin-4-yl)-2,2-difluoroethyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

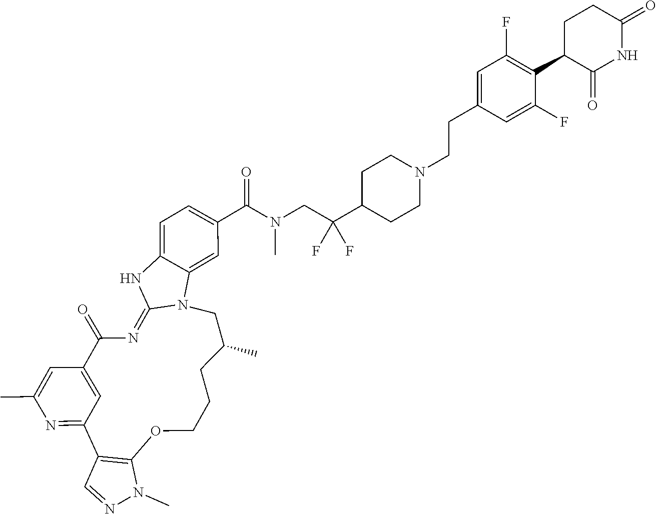 [SMILES: Cc1cc2cc(n1)-c1cnn(C)c1OCCC[C@@H](C)CN1/C(=N/C2=O)Nc2ccc(C(=O)N(C)CC(F)(F)C3CCN(CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.88 (s, 1H), 10.93 (s, 1H), 8.43 (s, 1H), 7.92 (s, 1H), 7.68 (s, 1H), 7.59-7.57 (m, 2H), 7.29 (s, 1H), 7.00-6.95 (m, 2H), 4.39-4.32 (m, 1H), 4.21-4.18 (m, 2H), 4.05-4.00 (m, 4H), 3.73 (s, 3H), 3.14-3.00 (m, 5H), 2.79-2.64 (m, 7H), 2.56 (s, 3H), 2.25-2.15 (m, 2H), 2.14-2.06 (m, 2H), 1.98-1.91 (m, 7H), 1.49-1.42 (m, 2H), 0.81 (d, J=5.5 Hz, 3H); [M+H]⁺=886.7.

### Example 276: (R,E)-N—(((S)-1-(4-((R)-2,6-dioxopiperidin-3-yl)-3,5-difluorophenethyl)piperidin-3-yl)methyl)-N,1¹, 2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

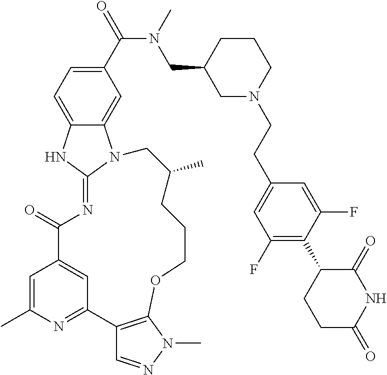 [SMILES: Cc1cc2cc(n1)-c1cnn(C)c1OCCC[C@@H](C)CN1/C(=N/C2=O)Nc2ccc(C(=O)N(C)C[C@H]3CCCN(CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.83 (s, 1H), 10.94 (s, 1H), 7.92 (s, 1H), 7.68 (s, 1H), 7.57 (s, 1H), 7.55 (s, 1H), 7.24 (s, 1H), 7.01 (s, 2H), 6.53 (s, 1H), 4.35 (s, 1H), 4.19 (d, J=11.8 Hz, 2H), 4.03-4.00 (m, 2H), 3.73 (s, 3H), 2.95 (s, 3H), 2.87-2.62 (m, 7H), 2.56 (s, 3H), 2.15-2.12 (m, 4H), 2.04-1.94 (m, 4H), 1.90 (s, 3H), 1.77 (s, 3H), 1.45-1.42 (m, 2H), 1.28-1.14 (m, 1H), 0.79 (d, J=6.4 Hz, 3H); [M+H]⁺=836.7.

### Example 278: (R,E)-N-(4-((4-((R)-2,6-dioxopiperidin-3-yl)-3,5-difluorophenethyl)(methyl)amino)cyclohexyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

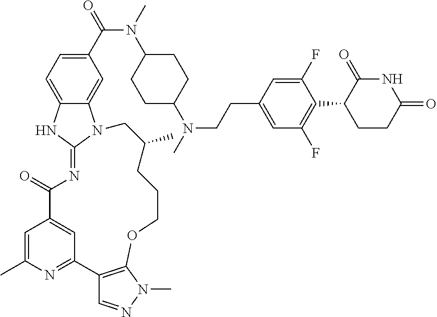 [SMILES: Cc1cc2cc(n1)-c1cnn(C)c1OCCC[C@@H](C)CN1/C(=N/C2=O)Nc2ccc(C(=O)N(C)C3CCC(N(C)CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.84 (s, 1H), 10.93 (s, 1H), 8.43 (s, 1H), 7.93 (s, 1H), 7.63 (s, 1H), 7.57 (d, J=5.9 Hz, 2H), 7.21 (s, 1H), 7.01 (d, J=12.0 Hz, 1H), 6.96 (s, 1H), 4.35 (d, J=4.4 Hz, 1H), 4.19 (d, J=11.4 Hz, 2H), 4.07-3.94 (m, 2H), 3.73 (s, 3H), 2.96-2.61 (m, 8H), 2.56 (s, 6H), 2.42-2.05 (m, 6H), 2.04-1.87 (m, 3H), 1.77 (s, 2H), 1.64 (s, 4H), 1.44 (d, J=5.2 Hz, 2H), 0.96 (s, 1H), 0.82 (d, J=5.5 Hz, 3H). [M+H]⁺=850.7

### Example 279: (R,E)-N-(2-(1-(4-((R)-2,6-dioxopiperidin-3-yl)-3,5-difluorophenyl)piperidin-4-yl)ethyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

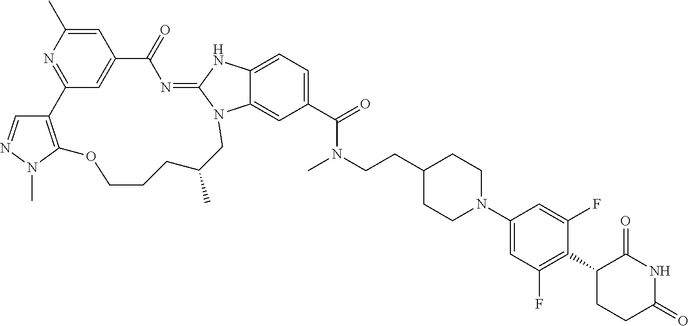 [SMILES: Cc1cc2cc(n1)-c1cnn(C)c1OCCC[C@@H](C)CN1/C(=N/C2=O)Nc2ccc(C(=O)N(C)CCC3CCN(c4cc(F)c([C@H]5CCC(=O)NC5=O)c(F)c4)CC3)cc21]

The titled compound was prepared in a manner similar to that in Example 260. ¹H NMR (500 MHz, DMSO) δ 12.86 (s, 1H), 10.85 (s, 1H), 8.43 (s, 1H), 7.92 (s, 1H), 7.68 (s, 1H), 7.58-7.56 (m, 2H), 7.26 (s, 1H), 6.62-6.54 (m, 2H), 4.40-4.32 (m, 1H), 4.21-4.15 (m, 1H), 4.05-4.00 (m, 3H), 3.73 (s, 3H), 3.64-3.49 (m, 3H), 3.05-2.91 (m, 4H), 2.87-2.72 (m, 5H), 2.56 (s, 3H), 2.26-2.16 (m, 1H), 2.10-1.81 (m, 5H), 1.61-1.36 (m, 5H), 1.29-1.20 (m, 2H), 0.82 (d, J=6.0 Hz, 3H); [M+H]⁺=822.8.

### Example 280: (R,E)-N-(2-(4-(4-((R)-2,6-dioxopiperidin-3-yl)-3,5-difluorophenyl)piperazin-1-yl)ethyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxyamide

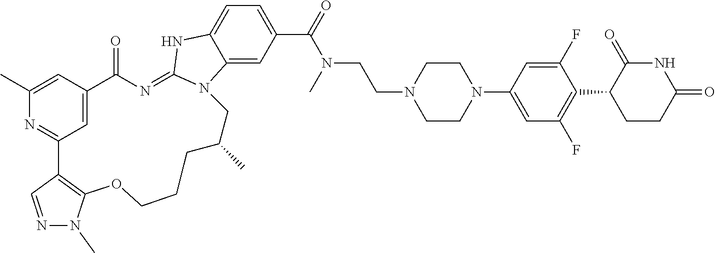 [SMILES: Cc1cc2cc(n1)-c1cnn(C)c1OCCC[C@@H](C)CN1/C(=N/C2=O)Nc2ccc(C(=O)N(C)CCN3CCN(c4cc(F)c([C@H]5CCC(=O)NC5=O)c(F)c4)CC3)cc21]

The titled compound was prepared in a manner similar to that in Example 260. ¹H NMR (500 MHz, DMSO) δ 12.85 (s, 1H), 10.86 (s, 1H), 8.42 (s, 1H), 7.92 (s, 1H), 7.67 (s, 1H), 7.57 (s, 1H), 7.56 (d, J=8.0 Hz, 1H), 7.27 (d, J=8.0 Hz, 1H), 6.63 (d, J=11.5 Hz, 2H), 4.39-4.31 (m, 1H), 4.21-4.15 (m, 1H), 4.05-3.95 (m, 3H), 3.73 (s, 3H), 3.24-3.15 (m, 2H), 3.12-3.07 (m, 2H), 3.05-3.03 (m, 3H), 2.85-2.71 (m, 3H), 2.67-2.56 (m, 4H), 2.34-2.17 (m, 3H), 2.13-1.89 (m, 5H), 1.49-1.39 (m, 1H), 1.28-1.23 (m, 4H), 0.80 (d, J=5.0 Hz, 3H); [M+H]⁺=823.7.

### Example 281: (R,E)-N-(2-(4-(4-((R)-2,6-dioxopiperidin-3-yl)-3,5-difluorophenyl)piperazin-1-yl)ethyl)-N-ethyl-1′,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

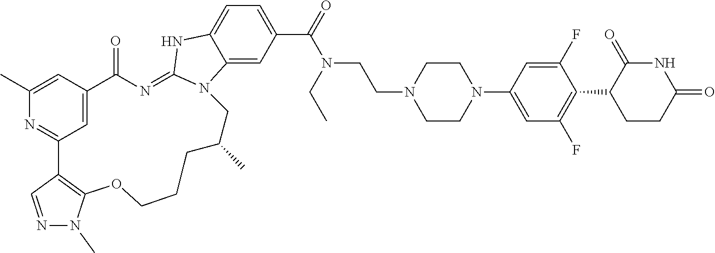

The titled compound was prepared in a manner similar to that in Example 260. ¹H NMR (500 MHz, DMSO) δ 12.85 (s, 1H), 10.86 (s, 1H), 8.43 (s, 1H), 7.92 (s, 1H), 7.65 (s, 1H), 7.58 (s, 1H), 7.57 (d, J=8.0 Hz, 1H), 7.23 (d, J=8.0 Hz, 1H), 6.63 (s, 2H), 4.39-4.32 (m, 1H), 4.20-4.17 (m, 1H), 4.06-3.95 (m, 3H), 3.73 (s, 3H), 3.58-3.44 (m, 4H), 3.24-3.03 (m, 6H), 2.84-2.72 (m, 3H), 2.56 (s, 3H), 2.32-2.18 (m, 3H), 2.14-2.03 (m, 1H), 1.99-1.88 (m, 3H), 1.48-1.39 (m, 1H), 1.23-1.04 (m, 5H), 0.81 (d, J=5.5 Hz, 3H); [M+H]⁺=837.9.

### Example 282: (R,E)-N-(2-(4-(4-((R)-2,6-dioxopiperidin-3-yl)-3,5-difluorophenyl)piperazin-1-yl)ethyl)-N-isopropyl -1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

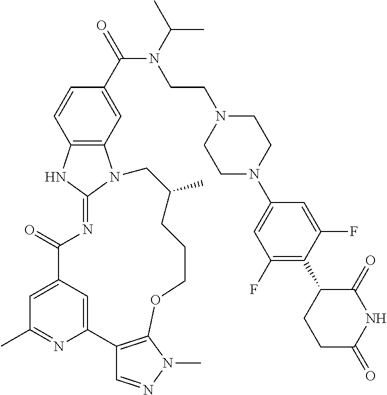 [SMILES: Cc1cc2cc(n1)-c1cnn(C)c1OCCC[C@@H](C)CN1/C(=N/C2=O)Nc2ccc(C(=O)N(CCN3CCN(c4cc(F)c([C@H]5CCC(=O)NC5=O)c(F)c4)CC3)C(C)C)cc21]

The titled compound was prepared in a manner similar to that in Example 260. ¹H NMR (500 MHz, DMSO) δ 12.84 (s, 1H), 10.87 (s, 1H), 8.43 (s, 1H), 7.92 (s, 1H), 7.63 (s, 1H), 7.57 (d, J=6.8 Hz, 2H), 7.19 (d, J=8.7 Hz, 1H), 6.64 (s, 2H), 4.37-4.31 (m, 1H), 4.19 (d, J=11.7 Hz, 1H), 4.02 (s, 3H), 3.73 (s, 3H), 3.45 (s, 1H), 3.29 (s, 2H), 3.22 (s, 3H), 2.78 (s, 2H), 2.64 (s, 3H), 2.56 (s, 3H), 2.48-2.40 (m, 2H), 2.22 (s, 1H), 2.07 (s, 1H), 2.01-1.88 (m, 3H), 1.44 (s, 1H), 1.31-1.07 (m, 9H), 0.82 (d, J=6.4 Hz, 3H). [M+H]⁺=851.7

### Example 283: (R,E)-N-(2-(4-(4-((R)-2,6-dioxopiperidin-3-yl)-3,5-difluorophenyl)piperidin-1-yl)ethyl)-N-isopropyl -1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

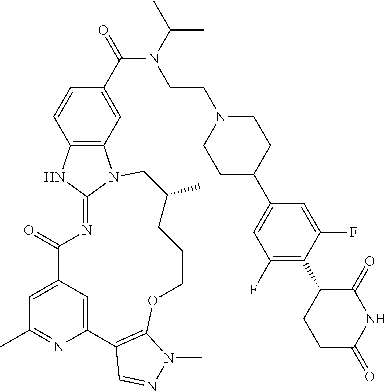 [SMILES: Cc1cc2cc(n1)-c1cnn(C)c1OCCC[C@@H](C)CN1/C(=N/C2=O)Nc2ccc(C(=O)N(CCN3CCC(c4cc(F)c([C@H]5CCC(=O)NC5=O)c(F)c4)CC3)C(C)C)cc21]

The titled compound was prepared in a manner similar to that in Example 260.

¹H NMR (500 MHz, DMSO) δ 12.84 (s, 1H), 10.94 (s, 1H), 8.43 (s, 1H), 7.92 (s, 1H), 7.64 (s, 1H), 7.57 (d, J=6.3 Hz, 2H), 7.19 (d, J=8.1 Hz, 1H), 7.03 (s, 2H), 4.36 (d, J=3.9 Hz, 1H), 4.19 (d, J=10.7 Hz, 2H), 4.09-3.97 (m, 2H), 3.73 (s, 3H), 3.42 (s, 1H), 3.29 (s, 3H), 3.09 (s, 1H), 2.80 (s, 2H), 2.65-2.54 (m, 5H), 2.49-2.37 (m, 3H), 2.25-1.98 (m, 3H), 2.03-1.89 (m, 3H), 1.83-1.56 (m, 4H), 1.44 (s, 1H), 1.29-1.07 (m, 6H), 0.82 (d, J=6.4 Hz, 3H). [M+H]⁺=850.7

### Example 285: (R,E)-N-(1-(2-(1-(4-((R)-2,6-dioxopiperidin-3-yl)-3,5-difluorophenyl)azetidin-3-yl)ethyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

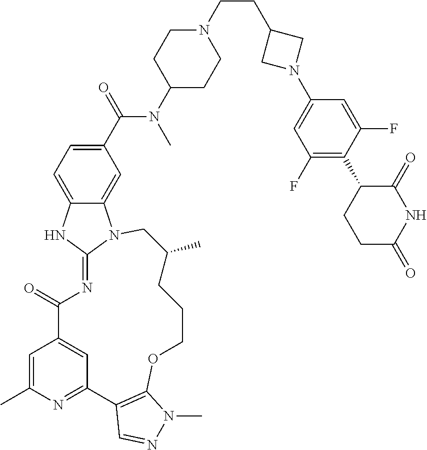 [SMILES: Cc1cc2cc(n1)-c1cnn(C)c1OCCC[C@@H](C)CN1/C(=N/C2=O)Nc2ccc(C(=O)N(C)C3CCN(CCC4CN(c5cc(F)c([C@H]6CCC(=O)NC6=O)c(F)c5)C4)CC3)cc21]

The titled compound was prepared in a manner similar to that in Example 260.

¹H NMR (500 MHz, DMSO) δ=12.85 (s, 1H), 10.84 (s, 1H), 8.43 (s, 1H), 7.92 (s, 1H), 7.66 (s, 1H), 7.57 (d, J=9.4 Hz, 2H), 7.21 (s, 1H), 6.07 (d, J=11.0 Hz, 2H), 4.36 (m, 1H), 4.19 (d, J=11.6 Hz, 1H), 4.08-3.97 (m, 3H), 3.91 (s, 2H), 3.73 (s, 3H), 3.41 (s, 2H), 3.01-2.63 (m, 9H), 2.56 (s, 3H), 2.21 (s, 2H), 2.14-2.00 (m, 3H), 1.94 (m, 4H), 1.78 (m, 3H), 1.67 (m, 2H), 1.49 (m, 3H), 0.81 (d, J=6.4 Hz, 3H).; [M+H]⁺=877.7.

### Example 286: (R,E)-N-(1-((1-(4-((R)-2,6-dioxopiperidin-3-yl)-3,5-difluorophenyl)azetidin-3-yl)methyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

### Step 1: methyl 1-(4-(2,6-bis(benzyloxy)pyridin-3-yl)-3,5-difluorophenyl)azetidine-3-carboxylate

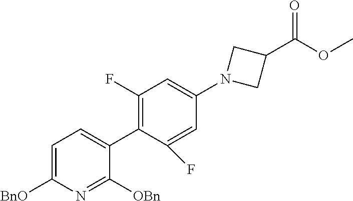 [SMILES: COC(=O)C1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (3.00 g, 6.22 mmol), methyl azetidine-3-carboxylate hydrochloride (1.41 g, 9.33 mmol), Cs₂CO₃ (6.06 g, 18.66 mmol) and RuPhos Pd G3 (520.7 mg, 0.622 mmol) in toluene (50 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+1]⁺=517.1.

### Step 2: (1-(4-(2,6-bis(benzyloxy)pyridin-3-yl)-3,5-difluorophenyl)azetidin-3-yl)methanol

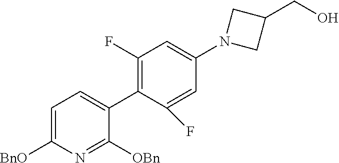 [SMILES: OCC1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiAlH₄ (1 M in THF, 4.27 mL, 4.27 mmol) dropwise at 0° C. Then the mixture was stirred for 2 hours, the reaction was quenched with water (10 mL) at 0° C. The resulting mixture was extracted with EtOAc (3×50 mL). The combined organic layers were washed with brine, dried over anhydrous Na₂SO₄. After filtration, the filtrate was concentrated under reduced pressure to afford the product (1.4 g, 87%) [M+1]⁺=489.2.

### Step 3: (R)-3-(2,6-difluoro-4-(3-(hydroxymethyl)azetidin-1-yl)phenyl)piperidine-2,6-dione

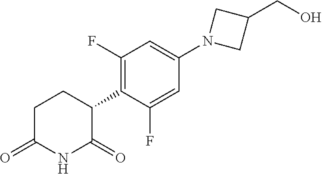 [SMILES: O=C1CC[C@H](c2c(F)cc(N3CC(CO)C3)cc2F)C(=O)N1]

To a solution of (1-(4-(2,6-bis(benzyloxy)pyridin-3-yl)-3,5-difluorophenyl)azetidin-3-yl)methanol (1.40 g, 2.87 mmol) in iPrOH (20 mL) and DCM (20 mL) was added Pd/C (1.0 g, 10% wt) which was stirred at room temperature under hydrogen atmosphere for 48 hours. The resulting mixture was filtered, the filter cake was washed with MeOH (20 mL). The residue was purified by SFC (IH (3*25 cm, Sum), 13% EtOH/87% CO₂, 100 bar, 2 ml/min) and the title compound corresponded to peak A @1.943 min/254 nm (450 mg, 51%). [M+1]⁺=311.3.

### Step 4: (R)-1-(4-(2,6-dioxopiperidin-3-yl)-3,5-difluorophenyl)azetidine-3-carbaldehyde

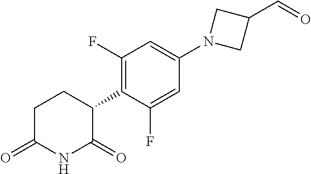 [SMILES: O=CC1CN(c2cc(F)c([C@H]3CCC(=O)NC3=O)c(F)c2)C1]

The titled compound (258 mg, 48%) was prepared in a manner similar to that in Example 1 step 4 from 3-(R)-3-(2,6-difluoro-4-(3-(hydroxymethyl)azetidin-1-yl)phenyl)piperidine-2,6-dione and IBX.

### Step 5: (R,E)-N-(1-((1-(4-((R)-2,6-dioxopiperidin-3-yl)-3,5-difluorophenyl)azetidin-3-yl)methyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

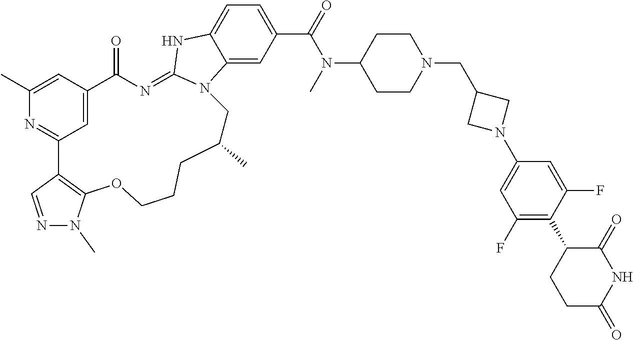 [SMILES: Cc1cc2cc(n1)-c1cnn(C)c1OCCC[C@@H](C)CN1/C(=N/C2=O)Nc2ccc(C(=O)N(C)C3CCN(CC4CN(c5cc(F)c([C@H]6CCC(=O)NC6=O)c(F)c5)C4)CC3)cc21]

The titled compound was prepared in a manner similar to that in Example 260. ¹H NMR (5(0) MHz, DMSO) δ 12.79 (s, 1H), 10.77 (s, 1H), 8.36 (s, 1H), 7.86 (s, 1H), 7.60 (s, 1H), 7.51 (s, 1H), 7.50 (d, J=8.5 Hz, 1H), 7.16 (s, 1H), 6.02 (s, 2H), 4.34-4.27 (m, 1H), 4.17-4.09 (m, 1H), 3.98-3.89 (m, 3H), 3.86-3.77 (m, 3H), 3.66 (s, 3H), 3.49-3.32 (m, 3H), 2.90-2.64 (m, 9H), 2.49 (s, 3H), 2.40-2.28 (m, 2H), 2.18-2.07 (m, 1H), 2.04-1.81 (m, 5H), 1.77-1.67 (m, 2H), 1.63-1.52 (m, 2H), 1.43-1.33 (m, 1H), 0.75 (d, J=5.5 Hz, 3H); [M+H]⁺=863.9.

### Example 287: (R,E)-N-(1-((1-(4-((R)-2,6-dioxopiperidin-3-yl)-3,5-difluorophenyl)piperidin-4-yl)methyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2.4)-pyridina-1(4,5)-pyrazolacycloundecaphane-5⁶-carboxamide

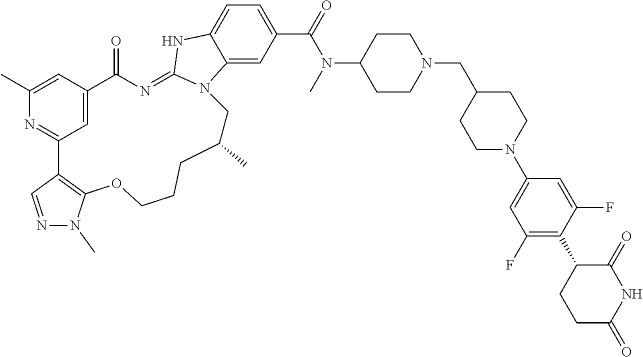 [SMILES: Cc1cc2cc(n1)-c1cnn(C)c1OCCC[C@@H](C)CN1/C(=N/C2=O)Nc2ccc(C(=O)N(C)C3CCN(CC4CCN(c5cc(F)c([C@H]6CCC(=O)NC6=O)c(F)c5)CC4)CC3)cc21]

The titled compound was prepared in a manner similar to that in Example 260. ¹H NMR (500 MHz, DMSO) δ 12.86 (s, 1H), 10.86 (s, 1H), 8.43 (s, 1H), 7.93 (s, 1H), 7.66 (s, 1H), 7.58 (s, 1H), 7.57 (d, J=8.5 Hz, 1H), 7.22 (s, 1H), 6.69 (d, J=13.0 Hz, 2H), 4.42-4.32 (m, 1H), 4.22-4.18 (m, 1H), 4.05-3.95 (m, 3H), 3.73 (s, 3H), 2.94-2.69 (m, 10H), 2.56 (s, 3H), 2.23-2.16 (m, 2H), 2.10-1.89 (m, 8H), 1.85-1.76 (m, 3H), 1.73-1.58 (m, 4H), 1.56-1.42 (m, 3H), 1.17-1.00 (m, 2H), 0.82 (d, J=6.5 Hz, 3H); [M+H]⁺=891.7.

### Example 288: (R,E)-N-(1′-(4-((R)-2,6-dioxopiperidin-3-yl)-3,5-difluorophenyl)-[1,4′-bipiperidin]-4-yl)-N-ethyl-1¹, 2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

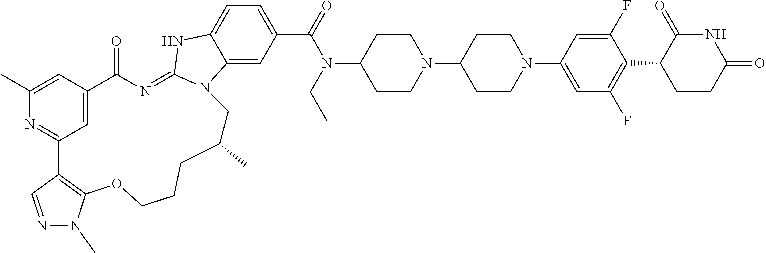 [SMILES: CCN(C(=O)c1ccc2c(c1)N1C[C@H](C)CCCOc3c(cnn3C)-c3cc(cc(C)n3)C(=O)/N=C/1N2)C1CCN(C2CCN(c3cc(F)c([C@H]4CCC(=O)NC4=O)c(F)c3)CC2)CC1]

The titled compound was prepared in a manner similar to that in Example 260. ¹H NMR (500 MHz, DMSO) δ 12.85 (s, 1H), 10.85 (s, 1H), 8.43 (s, 1H), 7.93 (s, 1H), 7.62 (s, 1H), 7.58 (s, 1H), 7.57 (d, J=7.0 Hz, 1H), 7.16 (d, J=7.0 Hz, 1H), 6.37 (d, J=12.0 Hz, 2H), 4.39-4.33 (m, 1H), 4.22-4.15 (m, 2H), 4.08-3.98 (m, 4H), 3.73 (s, 3H), 2.90-2.68 (m, 10H), 2.56 (s, 3H), 2.27-2.15 (m, 2H), 2.11-2.03 (m, 1H), 1.99-1.86 (m, 4H), 1.83-1.70 (m, 5H), 1.68-1.51 (m, 3H), 1.47-1.26 (m, 3H), 1.21-1.10 (m, 2H), 0.81 (d, J=6.0 Hz, 3H); [M+H]⁺=891.7.

### Example 289: (R,E)-N-(1′-(4-((R)-2,6-dioxopiperidin-3-yl)-3,5-difluorophenyl)-[1,4′-bipiperidin]-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

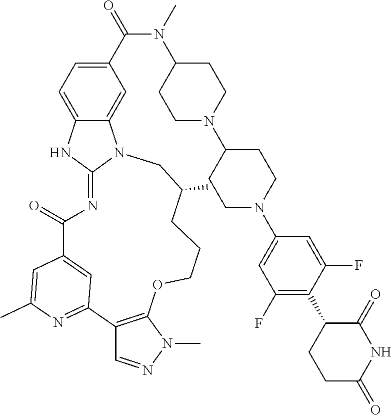 [SMILES: Cc1cc2cc(n1)-c1cnn(C)c1OCCC[C@@H](C)CN1/C(=N/C2=O)Nc2ccc(C(=O)N(C)C3CCN(C4CCN(c5cc(F)c([C@H]6CCC(=O)NC6=O)c(F)c5)CC4)CC3)cc21]

The titled compound was prepared in a manner similar to that in Example 260. ¹H NMR (500 MHz, DMSO) δ 12.83 (s, 1H), 10.85 (s, 1H), 8.43 (s, 1H), 7.92 (d, J=4.1 Hz, 1H), 7.65 (s, 1H), 7.56 (d, J=9.7 Hz, 2H), 7.21 (s, 1H), 6.58 (s, 2H), 4.36 (s, 1H), 4.19 (d, J=13.1 Hz, 2H), 4.03-4.00 (m, 4H), 3.73 (s, 3H), 2.87-2.77 (m, 10H), 2.56 (s, 3H), 2.21 (s, 2H), 2.07 (s, 1H), 1.95 (s, 3H), 1.78-1.68 (m, 8H), 1.47-1.45 (m, 4H), 0.82 (d, J=6.4 Hz, 3H); [M+H]⁺=877.7.

### Example 290: (R,E)-N-(1-(1-(4-((R)-2,6-dioxopiperidin-3-yl)-3,5-difluorophenyl)azetidin-3-yl)piperidin-4-yl)-N,1′,2⁶,7-tetramethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carboxamide

### Step 1: 1-(4-(2,6-bis(benzyloxy)pyridin-3-yl)-3,5-difluorophenyl)azetidin-3-ol

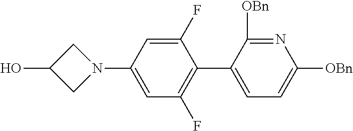 [SMILES: OC1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (2 g, 4.16 mmol), azetidin-3-ol hydrochloride (1.36 g, 12.47 mmol), Pd₂(dba)₃ (380 mg, 0.42 mmol), 9,9-dimethyl-4,5-bis(diphenylphosphino)xanthene (486 mg, 0.84 mmol), Cs₂CO₃ (4.07 g, 12.47 mmol) in 1,4-dioxane (40 mL) was stirred in a 100 ml flask at 100° C. under atmosphere of N2 for 16 h. After being cooled to room temperature, the mixture was filtrated through a pad of celite. The filtrate was concentrated under reduced pressure, the residue was purified by silica gel column eluting with PE/EA (2:1) to give the target product (1.8 g, 91%). [M+H]⁺=475.3.

### Step 2: 3-(2,6-difluoro-4-(3-hydroxyazetidine-1-yl)phenyl)piperidine-2,6-dione

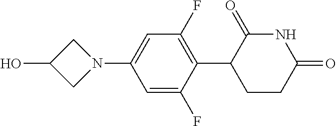 [SMILES: O=C1CCC(c2c(F)cc(N3CC(O)C3)cc2F)C(=O)N1]

A mixture of 1-(4-(2,6-bis(benzyloxy)pyridin-3-yl)-3,5-difluorophenyl)azetidin-3-ol (1.8 g, 3.79 mmol) in iPrOH (30 mL) and DMF (30 mL) was added Pd/C (10%, w/w, 1 g) under N2(g). The resulting mixture was stirred under H₂ atmosphere for overnight until LC-MS indicated all the starting material was consumed. The resulting solution was filtered and the filtrate was concentrated to give the desire product (0.9 g, 80%). [M+H]⁺=297.2

### Step 3: 3-(2,6-difluoro-4-(3-oxoazetidin-1-yl)phenyl)piperidine-2,6-dione

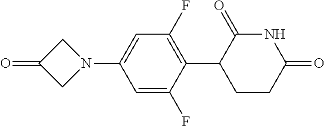 [SMILES: O=C1CN(c2cc(F)c(C3CCC(=O)NC3=O)c(F)c2)C1]

A mixture of 3-(2,6-difluoro-4-(3-hydroxyazetidin-1-yl)phenyl)piperidine-2,6-dione (900 mg, 3.03 mmol) and DMP (1.93 g, 4.55 mmol) in DCM (10 mL) was stirred in a flask at room temperature for 2 hours. The reaction was quenched with con aq Na₂S₂O₃ and the mixture was extracted with DCM, washed three times with saturated aqueous NaCl and twice with saturated aqueous NaHCO₃. The organic layer was dried over anhydrous Na₂SO₄ and evaporated in vacuum to afford the crude product (1.1 g). [M+H]⁺=295.2.

### Step 4: (R,E)-N-(1-(1-(4-((R)-2,6-dioxopiperidin-3-yl)-3,5-difluorophenyl)azetidin-3-yl)piperidin-4-yl)-N,1¹,2⁶,7-tetramethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-carboxamide

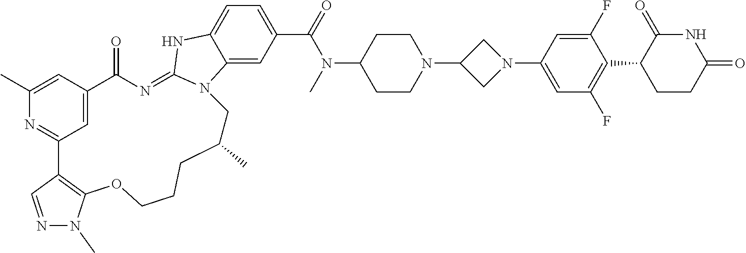 [SMILES: Cc1cc2cc(n1)-c1cnn(C)c1OCCC[C@@H](C)CN1/C(=N/C2=O)Nc2ccc(C(=O)N(C)C3CCN(C4CN(c5cc(F)c([C@H]6CCC(=O)NC6=O)c(F)c5)C4)CC3)cc21]

The titled compound was prepared in a manner similar to that in Example 260. Chiral separation by HPLC afforded the title compound. HPLC conditions: Column: CHIRALPAK IE-3; Column Size: 2 cm×25 cm, Sum; Mobile Phase: MtBE (0.1% TFA):(MeOH:DCM=1:1)=50:50; Flow Rate: 1.0 mL/min; Retention Time: 2.051 min. ¹H NMR (500 MHz, DMSO) δ 12.84 (s, 1H), 10.85 (s, 1H), 8.43 (s, 1H), 7.92 (s, 1H), 7.66 (s, 1H), 7.57 (s, 1H), 7.56 (d, J=8.5 Hz, 1H), 7.22 (s, 1H), 6.08 (s, 2H), 4.39-4.31 (m, 1H), 4.22-4.17 (m, 1H), 4.05-3.97 (m, 4H), 3.73 (s, 3H), 3.60-3.48 (m, 5H), 2.87-2.75 (m, 8H), 2.56 (s, 3H), 2.25-2.15 (m, 1H), 2.08-1.88 (m, 6H), 1.84-1.77 (m, 2H), 1.72-1.67 (m, 1H), 1.60-1.50 (m, 1H), 1.48-1.42 (m, 1H), 0.80 (d, J=5.5 Hz, 3H); [M+H]⁺=849.7.

### Example 291: (R,E)-N—((R)-1-(4-((R)-2,6-dioxopiperidin-3-yl)-3,5-difluorophenethyl)pyrrolidin-3-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

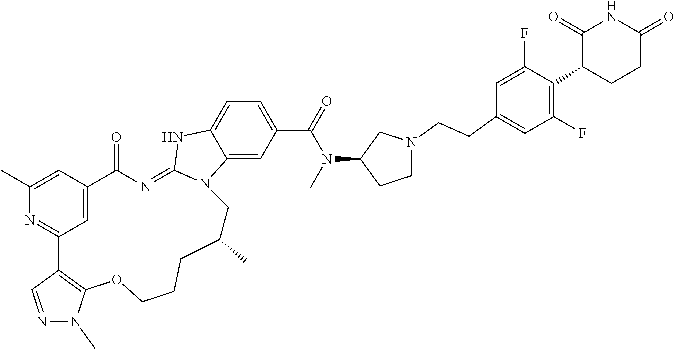 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.85 (s, 1H), 10.93 (s, 1H), 8.43 (s, 1H), 7.93 (s, 1H), 7.67-7.57 (m, 3H), 7.22 (s, 1H), 7.03 (d, J=10.5 Hz, 2H), 4.40-4.31 (m, 1H), 4.20-4.18 (m, 2H), 4.05-3.96 (m, 2H), 3.73 (s, 3H), 2.88-2.76 (m, 10H), 2.69-2.61 (m, 2H), 2.56 (s, 3H), 2.45-2.34 (m, 1H), 2.28-2.15 (m, 2H), 2.14-2.05 (m, 2H), 1.98-1.88 (m, 4H), 1.84-1.80 (m, 1H), 1.50-1.40 (m, 1H), 0.82 (d, J=6.0 Hz, 3H); [M+H]⁺=808.7.

### Example 292: (R,E)-N—((R)-1-(4-((R)-2,6-dioxopiperidin-3-yl)-3,5-difluorophenethyl)azepan-4-yl)-N,1¹,2⁶,7-tetra methyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

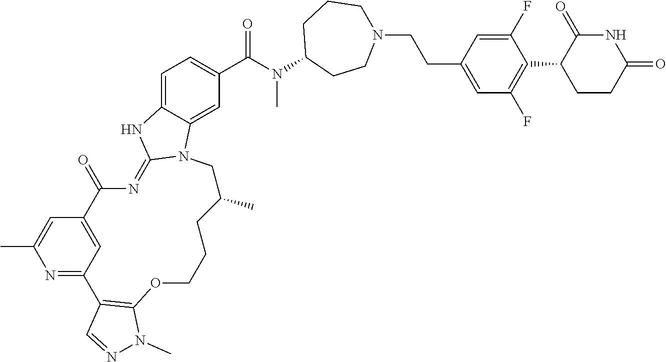 [SMILES: Cc1cc2cc(n1)-c1cnn(C)c1OCCC[C@@H](C)CN1/C(=N/C2=O)Nc2ccc(C(=O)N(C)[C@@H]3CCCN(CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.84 (s, 1H), 10.91 (s, 1H), 8.43 (s, 1H), 7.92 (s, 1H), 7.67-7.63 (m, 1H), 7.57 (s, 1H), 7.56 (d, J=10.5 Hz, 1H), 7.23-7.19 (m, 1H), 7.06-6.89 (m, 2H), 4.40-4.32 (m, 1H), 4.20-4.10 (m, 2H), 4.03-3.95 (m, 2H), 3.73 (s, 3H), 2.92-2.68 (m, 9H), 2.56 (s, 3H), 2.50-2.36 (m, 7H), 2.27-2.16 (m, 1H), 2.13-1.90 (m, 4H), 1.84-1.70 (m, 4H), 1.66-1.56 (m, 1H), 1.50-1.40 (m, 1H), 0.82 (d, J=6.0 Hz, 3H); [M+H]⁺=836.4.

### Example 293: (R,E)-N—((3aR,6aR)-2-(4-((R)-2,6-dioxopiperidin-3-yl)-3,5-difluorophenethyl)octahydrocyclopenta[c]pyrrol-5-yl)-N,1¹,2⁶,7-tetramethyl-3-oxo-5²,5¹-dihydro-1¹H,5¹H-11-oxa-4-aza-5(2,1)-benzo[d]imidazola-2(2,4)-pyridina-1(4,5)-pyrazolacycloundecaphane-5⁶-carboxamide

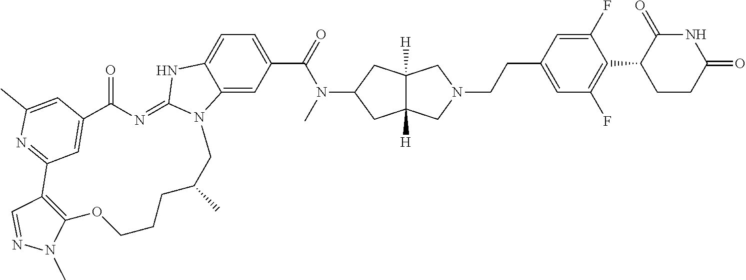 [SMILES: Cc1cc2cc(n1)-c1cnn(C)c1OCCC[C@@H](C)CN1/C(=N/C2=O)Nc2ccc(C(=O)N(C)C3C[C@H]4CN(CCc5cc(F)c([C@H]6CCC(=O)NC6=O)c(F)c5)C[C@@H]4C3)cc21]

The titled compound was prepared in a manner similar to that in Example 260.

¹H NMR (500 MHz, DMSO) δ 12.90 (s, 1H), 10.96 (s, 1H), 9.73 (s, 1H), 8.49 (s, 1H), 8.01 (s, 1H), 7.71 (s, 1H), 7.65 (s, 1H), 7.58 (d, J=8.2 Hz, 1H), 7.28 (d, J=8.8 Hz, 1H), 7.10 (d, J=9.6 Hz, 2H), 4.37 (d, J=4.2 Hz, 2H), 4.27-4.18 (m, 3H), 4.11-4.02 (m, 3H), 3.83 (s, 2H), 3.45 (s, 2H), 3.32 (s, 1H), 3.08 (s, 1H), 2.95 (s, 2H), 2.85-2.62 (m, 7H), 2.61 (s, 3H), 2.54 (s, 1H), 2.19 (d, J=11.0 Hz, 1H), 2.16-2.04 (in, 1H), 2.01-1.90 (m, 5H), 1.73-1.54 (m, 2H), 1.4.6 (s, 1H), 0.83 (d, J=6.3 Hz, 3H). [M+H]⁺=848.8.

### Example 295: (R)-3-(2,6-difluoro-4-(2-(menthyl((R)-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)pyrrolidin-3-yl)amino)ethyl)phenyl)piperidine-2,6-dione

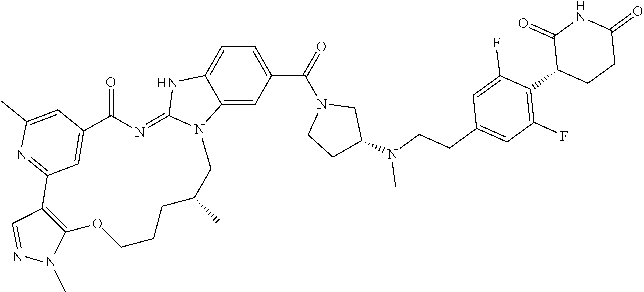 [SMILES: Cc1cc2cc(n1)-c1cnn(C)c1OCCC[C@@H](C)CN1/C(=N/C2=O)Nc2ccc(C(=O)N3CC[C@@H](N(C)CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.86 (s, 1H), 10.93 (d, J=10.6 Hz, 1H), 8.43 (s, 1H), 7.93 (s, 1H), 7.79 (d, J=12.3 Hz, 1H), 7.56 (d, J=9.5 Hz, 2H), 7.39 (t, J=7.7 Hz, 1H), 7.06 (d, J=10.3 Hz, 1H), 6.98 (t, J=10.9 Hz, 1H), 4.36 (s, 1H), 4.18 (d, J=10.9 Hz, 2H), 4.02 (d, J=24.3 Hz, 2H), 3.83-3.71 (m, 3H), 3.69-3.52 (m, 2H), 3.47 (s, 1H), 3.40-3.17 (m, 3H), 3.04 (d, J=11 Hz, 1H), 2.79 (d, J=12.7 Hz, 3H), 2.71 (dd. J=15.4, 7.0 Hz, 2H), 2.56 (s, 3H), 2.29 (s, 1H), 2.25-1.84 (m, 8H), 1.74 (d, J=10.2 Hz, 1H), 1.45 (s, 1H), 0.80 (t, J=6.7 Hz, 3H).

[M+H]⁺=808.7.

### Example 296: (R)-3-(2,6-difluoro-4-(2-(((R)-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5′-carbonyl)pyrrolidin-3-yl)amino)ethyl)phenyl)piperidine-2,6-dione

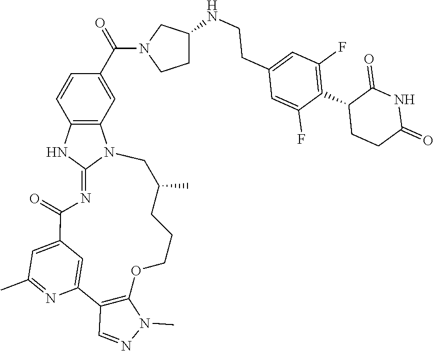 [SMILES: Cc1cc2cc(n1)-c1cnn(C)c1OCCC[C@@H](C)CN1/C(=N/C2=O)Nc2ccc(C(=O)N3CC[C@@H](NCCc4cc(F)c([C@H]5CCC(=O)NC5=O)c(F)c4)C3)cc21]

The titled compound was prepared in a manner similar to that in Example 260. ¹H NMR (500 MHz, DMSO) δ 13.17-12.47 (m, 1H), 10.93 (d, J=14.1 Hz, 1H), 8.43 (s, 1H), 7.92 (s, 1H), 7.78 (s, 1H), 7.56 (d, J=11.3 Hz, 2H), 7.39 (d, J=8.0 Hz, 1H), 7.04 (d, J=9.9 Hz, 1H), 6.94 (d, J=10.2 Hz, 1H), 4.35 (s, 1H), 4.27-4.14 (m, 2H), 4.10-3.90 (m, 2H), 3.73 (s, 3H), 3.67-3.42 (m, 4H), 2.78-2.75 (m, 5H), 2.71-2.55 (m, 6H), 2.22-1.81 (m, 6H), 1.80-1.60 (m, 1H), 1.51-1.36 (m, 1H), 0.91-0.74 (m, 3H); [M+H]⁺=794.7.

### Example 297: (R)-3-(2,6-difluoro-4-(2-(methyl((S)-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)pyrrolidin-3-yl)amino)ethyl)phenyl)piperidine-2,6-dione

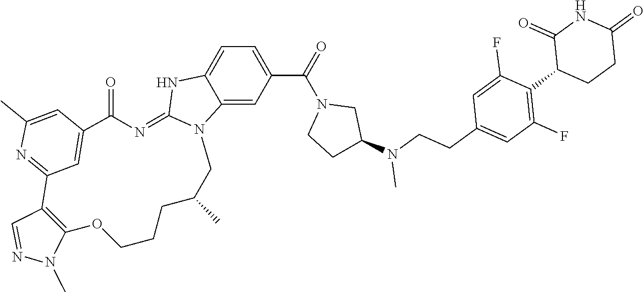 [SMILES: Cc1cc2cc(n1)-c1cnn(C)c1OCCC[C@@H](C)CN1/C(=N/C2=O)Nc2ccc(C(=O)N3CC[C@H](N(C)CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.86 (s, 1H), 10.93 (d, J=10.6 Hz, 1H), 8.43 (s, 1H), 7.93 (s, 1H), 7.79 (d, J=12.3 Hz, 1H), 7.56 (d, J=9.5 Hz, 2H), 7.39 (t, J=7.7 Hz, 1H), 7.06 (d, J=10.3 Hz, 1H), 6.98 (t, J 10.9 Hz, 1H), 4.36 (s, 1H), 4.18 (d, J=10.9 Hz, 2H), 4.02 (d, J=24.3 Hz, 2H), 3.83-3.71 (m, 3H), 3.69-3.52 (m, 2H), 3.47 (s, 1H), 3.40-3.17 (m, 3H), 3.04 (d, J=11 Hz, 1H), 2.79 (d, J=12.7 Hz, 3H), 2.71 (dd. J=15.4, 7.0 Hz, 2H), 2.56 (s, 3H), 2.29 (s, 1H), 2.25-1.84 (m, 8H), 1.74 (d, J=10.2 Hz, 1H), 1.45 (s, 1H), 0.80 (t, J=6.7 Hz, 3H).

[M+H]⁺=808.7.

### Example 298: (R)-3-(2,6-difluoro-4-(2-(((S)-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)pyrrolidin-3-yl)amino)ethyl)phenyl)piperidine-2,6-dione

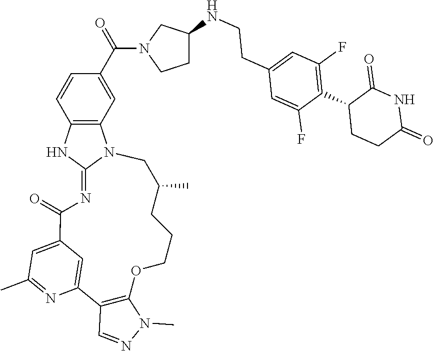 [SMILES: Cc1cc2cc(n1)-c1cnn(C)c1OCCC[C@@H](C)CN1/C(=N/C2=O)Nc2ccc(C(=O)N3CC[C@H](NCCc4cc(F)c([C@H]5CCC(=O)NC5=O)c(F)c4)C3)cc21]

The titled compound was prepared in a manner similar to that in Example 260. ¹H NMR (500 MHz, DMSO) δ 13.17-12.47 (m, 1H), 10.93 (d, J=14.1 Hz, 1H), 8.43 (s, 1H), 7.92 (s, 1H), 7.78 (s, 1H), 7.56 (d, J=11.3 Hz, 2H), 7.39 (d, J=8.0 Hz, 1H), 7.04 (d, J=9.9 Hz, 1H), 6.94 (d, J=10.2 Hz, 1H), 4.35 (s, 1H), 4.27-4.14 (m, 2H), 4.10-3.90 (m, 2H), 3.73 (s, 3H), 3.67-3.42 (m, 4H), 2.78-2.75 (m, 5H), 2.71-2.55 (m, 6H), 2.22-1.81 (m, 6H), 1.80-1.60 (m, 1H), 1.51-1.36 (m, 1H), 0.91-0.74 (m, 3H); [M+H]⁺=794.7.

### Example 299: (R)-3-(2,6-difluoro-4-(2-(methyl(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperidin-4-yl)amino)ethyl)phenyl)piperidine-2,6-dione

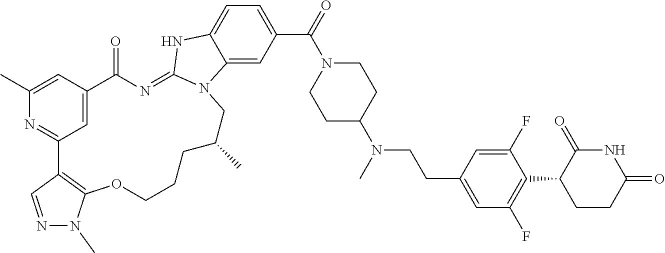 [SMILES: Cc1cc2cc(n1)-c1cnn(C)c1OCCC[C@@H](C)CN1/C(=N/C2=O)Nc2ccc(C(=O)N3CCC(N(C)CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.84 (s, 1H), 10.94 (s, 1H), 8.43 (s, 1H), 7.92 (s, 1H), 7.68 (s, 1H), 7.57 (s, 1H), 7.56 (d, J=8.0 Hz, 1H), 7.25 (d, J=8.5 Hz, 1H), 7.03 (d, J=10.0 Hz, 2H), 4.40-4.32 (m, 1H), 4.21-4.18 (m, 2H), 4.07-4.00 (m, 2H), 3.73 (s, 3H), 3.05-2.91 (m, 2H), 2.85-2.64 (m, 10H), 2.56 (s, 3H), 2.27 (s, 3H), 2.23-2.17 (m, 1H), 2.13-2.08 (m, 1H), 2.03-1.89 (m, 3H), 1.82-1.54 (m, 2H), 1.50-1.38 (m, 3H), 0.82 (d, J=6.0 Hz, 3H); [M+H]⁺=822.9.

### Example 300: (R)-3-(2,6-difluoro-4-(2-((1-((R,E)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperidin-4-yl) amino)ethyl)phenyl)piperidine-2,6-dione

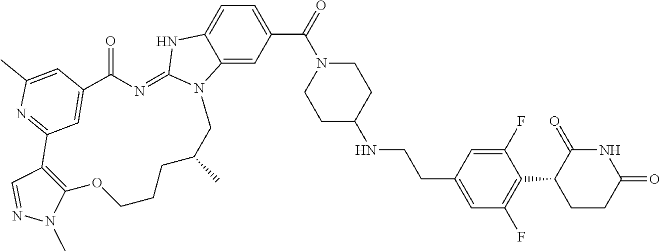 [SMILES: Cc1cc2cc(n1)-c1cnn(C)c1OCCC[C@@H](C)CN1/C(=N/C2=O)Nc2ccc(C(=O)N3CCC(NCCc4cc(F)c([C@H]5CCC(=O)NC5=O)c(F)c4)CC3)cc21]

The titled compound was prepared in a manner similar to that in Example 260. ¹H NMR (500 MHz, DMSO) δ 10.94 (s, 1H), 8.43 (s, 1H), 7.92 (s, 1H), 7.66 (s, 1H), 7.56 (d, J=8.2 Hz, 2H), 7.24 (d, J=8.2 Hz, 1H), 7.02 (d, J=10.0 Hz, 2H), 4.36 (d, J=4.6 Hz, 1H), 4.19 (d, J=12.3 Hz, 2H), 4.05-3.98 (m, 2H), 3.73 (s, 3H), 3.56 (s, 1H), 3.03 (s, 2H), 2.86-2.71 (m, 8H), 2.56 (s, 3H), 2.54 (s, 1H), 2.48-2.43 (m, 1H), 2.21 (s, 1H), 2.12 (dd, J=13.2, 3.7 Hz, 1H), 2.02-1.88 (m, 4H), 1.83-1.71 (m, 1H), 1.44 (s, 1H), 1.27 (s, 2H), 0.82 (d, J=6.4 Hz, 3H), [M+H]⁺=808.7.

### Example 301: (R)-3-(2,6-difluoro-4-(4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazin-1-yl) butyl)phenyl)piperidine-2,6-dione

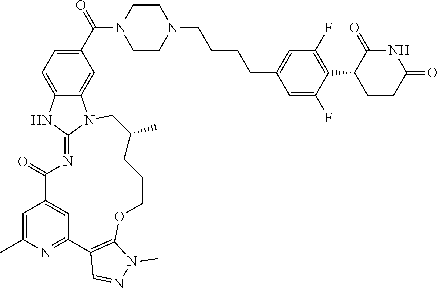 [SMILES: Cc1cc2cc(n1)-c1cnn(C)c1OCCC[C@@H](C)CN1/C(=N/C2=O)Nc2ccc(C(=O)N3CCN(CCCCc4cc(F)c([C@H]5CCC(=O)NC5=O)c(F)c4)CC3)cc21]

The titled compound was prepared in a manner similar to that in Example 260. ¹H NMR (500 MHz, DMSO) δ 12.86 (s, 1H), 10.94 (s, 1H), 8.43 (s, 1H), 7.92 (d, J=1.7 Hz, 1H), 7.67 (s, 1H), 7.57 (d, J=6.3 Hz, 2H), 7.25 (d, J=8.0 Hz, 1H), 6.98 (d, J=10.5 Hz, 2H), 4.35 (s, 1H), 4.19 (d, J=12.2 Hz, 2H), 4.06-3.97 (m, 2H), 3.73 (s, 3H), 3.59 (s, 2H), 2.80 (t, J=12.4 Hz, 2H), 2.60 (t, J=7.4 Hz, 3H), 2.56 (s, 3H), 2.40-2.32 (m, 7H), 2.25-2.05 (m, 2H), 1.98-1.91 (m, 4H), 1.59 (d, J=7.1 Hz, 2H), 1.45 (s, 3H), 0.81 (d, J=5.8 Hz, 3H); [M+H]⁺=822.5.

### Example 303: (R)-3-(2,6-difluoro-4-(4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazin-1-yl) piperidin-1-yl)phenyl)piperidine-2,6-dione

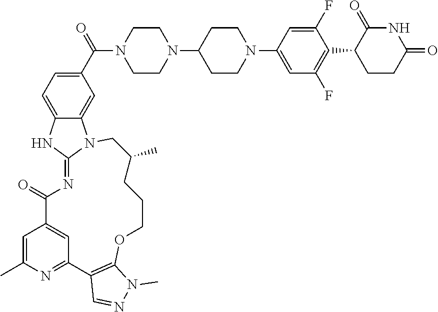 [SMILES: Cc1cc2cc(n1)-c1cnn(C)c1OCCC[C@@H](C)CN1/C(=N/C2=O)Nc2ccc(C(=O)N3CCN(C4CCN(c5cc(F)c([C@H]6CCC(=O)NC6=O)c(F)c5)CC4)CC3)cc21]

The titled compound was prepared in a manner similar to that in Example 260. ¹H NMR (500 MHz, DMSO) δ 12.86 (s, 1H), 10.86 (s, 1H), 8.43 (s, 1H), 7.92 (s, 1H), 7.67 (s, 1H), 7.57 (d, J=6.2 Hz, 2H), 7.26 (d, J=8.1 Hz, 1H), 6.59-6.54 (m, 2H), 4.38 (s, 1H), 4.19 (d, J=11.8 Hz, 1H), 4.08-3.96 (m, 3H), 3.80-3.76 (m, 2H), 3.70 (s, 3H), 3.69-3.56 (m, 1H), 2.77-2.73 (m, 4H), 2.67-2.53 (m, 6H), 2.49-2.38 (m, 6H), 2.29-2.15 (m, 1H), 2.15-2.02 (m, 1H), 2.02-1.85 (m, 3H), 1.81-1.79 (m, 2H), 1.53-1.38 (m, 3H), 0.82 (d, J=6.3 Hz, 3H); [M+H]⁺=849.7.

### Example 304: (R)-3-(2,6-difluoro-4-(2-((3aS,7aR)-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)octahydro-5H-pyrrolo[3,2-c]pyridin-5-yl)ethyl)phenyl)piperidine-2,6-dione

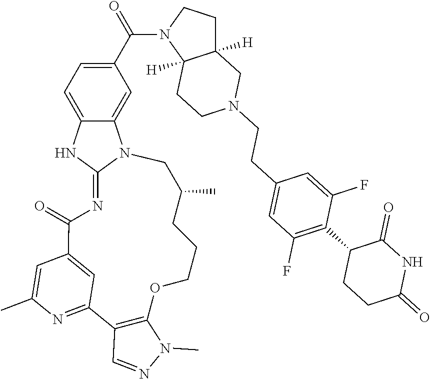 [SMILES: Cc1cc2cc(n1)-c1cnn(C)c1OCCC[C@@H](C)CN1/C(=N/C2=O)Nc2ccc(C(=O)N3CC[C@H]4CN(CCc5cc(F)c([C@H]6CCC(=O)NC6=O)c(F)c5)CC[C@H]43)cc21]

The titled compound was prepared in a manner similar to that in Example 260. ¹H NMR (500 MHz, DMSO) δ 12.85 (s, 1H), 10.94 (s, 1H), 8.43 (s, 1H), 7.92 (s, 1H), 7.77 (s, 1H), 7.63-7.49 (m, 2H), 7.38-7.29 (m-, 1H), 7.05-6.91 (m, 2H), 4.36 (d, J=4.0 Hz, 1H), 4.19 (d, J=12.3 Hz, 2H), 4.00 (s, 2H), 3.73 (s, 3H), 3.66-3.56 (m, 2H), 2.94-2.63 (m, 8H), 2.56 (s, 3H), 2.54-2.50 (m, 4H), 2.12 (d, J=12.9 Hz, 4H), 1.99-1.79 (m, 6H), 1.45 (s, 1H), 0.82 (t, J=7.1 Hz, 3H); [M+H]⁺=834.5.

### Example 305: (R)-3-(2,6-difluoro-4-(2-((1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)azetidin-3-yl)amino)ethyl)phenyl)piperidine-2,6-dione

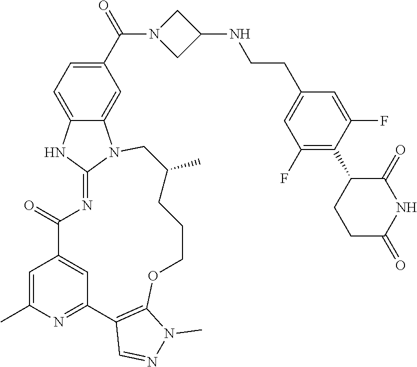 [SMILES: Cc1cc2cc(n1)-c1cnn(C)c1OCCC[C@@H](C)CN1/C(=N/C2=O)Nc2ccc(C(=O)N3CC(NCCc4cc(F)c([C@H]5CCC(=O)NC5=O)c(F)c4)C3)cc21]

The titled compound was prepared in a manner similar to that in Example 260. 1H NMR (500 MHz, DMSO) δ 12.85 (s, 1H), 10.94 (s, 1H), 8.43 (s, 1H), 7.92 (s, 1H), 7.81 (s, 1H), 7.57 (d, J=4.4 Hz, 2H), 7.51 (d, J=7.8 Hz, 1H), 7.00 (d, J=10.3 Hz, 2H), 4.46 (s, 1H), 4.35 (s, 1H), 4.19 (d, J=11.9 Hz, 3H), 4.09-3.97 (m, 3H), 3.77 (s, 1H), 3.73 (s, 3H), 3.63 (s, 1H), 3.44 (s, 1H), 2.84-2.71 (m, 6H), 2.56 (s, 3H), 2.21 (s, 1H), 2.17-2.06 (m, 1H), 2.02-1.91 (m, 3H), 1.47 (s, 1H), 0.82 (d, J=6.3 Hz, 3H). [M+H]⁺=780.7

### Example 306: (R)-3-(2,6-difluoro-4-(2-(methyl(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)azetidin-3-yl)amino)ethyl)phenyl)piperidine-2,6-dione

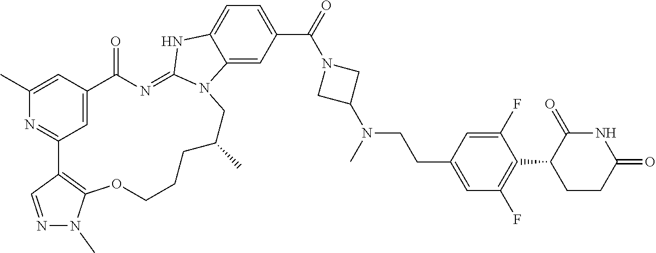 [SMILES: Cc1cc2cc(n1)-c1cnn(C)c1OCCC[C@@H](C)CN1/C(=N/C2=O)Nc2ccc(C(=O)N3CC(N(C)CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.89 (s, 1H), 10.94 (s, 1H), 8.43 (s, 1H), 7.92 (s, 1H), 7.83 (s, 1H), 7.57 (s, 2H), 7.52 (s, 1H), 7.05 (d, J=10.0 Hz, 2H), 4.38-4.30 (m, 2H), 4.20-4.07 (m, 6H), 4.04-3.97 (m, 1H), 3.87-3.82 (m, 1H), 3.73 (s, 3H), 2.85-2.70 (m, 5H), 2.56 (s, 3H), 2.55-2.50 (m, 3H), 2.19 (s, 3H), 2.14-2.04 (m, 1H), 2.01-1.87 (m, 3H), 1.50-1.42 (m, 1H), 0.82 (d, J=5.5 Hz, 3H); [M+H]⁺=794.9.

### Example 307: (R,E)-N-(1-(4-((R)-2,6-dioxopiperidin-3-yl)-3,5-difluorophenethyl)azetidin-3-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazola cycloundecaphane-5⁶-carboxamide

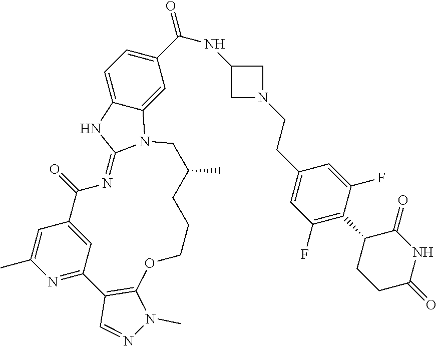 [SMILES: Cc1cc2cc(n1)-c1cnn(C)c1OCCC[C@@H](C)CN1/C(=N/C2=O)Nc2ccc(C(=O)NC3CN(CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.90 (s, 1H), 10.95 (s, 1H), 8.76 (d, J=6.6 Hz, 1H), 8.43 (s, 1H), 8.07 (s, 1H), 7.93 (s, 1H), 7.79 (d, J=8.3 Hz, 1H), 7.56 (d, J=10.5 Hz, 2H), 7.03 (d, J=10.0 Hz, 2H), 4.49 (dd, J=13.7, 6.9 Hz, 1H), 4.36 (d, J=4.6 Hz, 1H), 4.25-4.18 (m, 2H), 4.02-3.94 (m, 2H), 3.74 (s, 3H), 3.63 (t, J=6.7 Hz, 2H), 3.11-3.05 (m, 2H), 2.85-2.78 (m, 2H), 2.72 (d, J=6.7 Hz, 2H), 2.65-2.61 (m, 3H), 2.56 (s, 2H), 2.55 (s, 1H), 2.23 (s, 1H), 2.17-2.11 (m, 1H), 2.05-1.98 (m, 2H), 1.93 (d, J=10.7 Hz, 1H), 1.49 (s, 1H), 0.83 (d, J=6.5 Hz, 3H); [M+H]⁺=780.5.

### Example 308: (R,E)-N-(1-(1-(4-((R)-2,6-dioxopiperidin-3-yl)-3,5-difluorophenethyl)azetidin-3-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

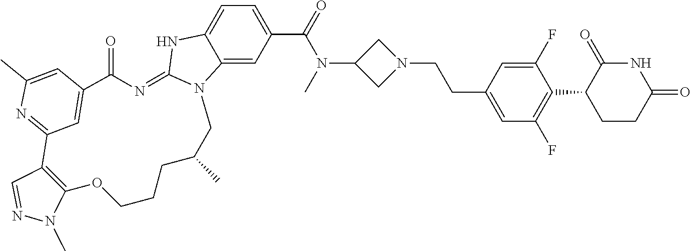 [SMILES: Cc1cc2cc(n1)-c1cnn(C)c1OCCC[C@@H](C)CN1/C(=N/C2=O)Nc2ccc(C(=O)N(C)C3CN(CCc4cc(F)c([C@H]5CCC(=O)NC5=O)c(F)c4)C3)cc21]

The titled compound was prepared in a manner similar to that in Example 260. ¹H NMR (500 MHz, DMSO) δ 12.86 (s, 1H), 10.93 (s, 1H), 8.43 (s, 1H), 7.93 (s, 1H), 7.75-7.45 (m, 3H), 7.23 (s, 1H), 6.99 (s, 2H), 4.36 (s, 1H), 4.18 (s, 2H), 4.01 (s, 2H), 3.73 (s, 3H), 3.08 (d, J=12 Hz, 4H), 2.80 (s, 3H), 2.64 (s, 3H), 2.55 (d, J=9.0 Hz, 6H), 2.36 (s, 2H), 2.22 (s, 1H), 2.11 (d, J=12.5 Hz, 1H), 1.99 (s, 3H), 1.45 (s, 1H), 0.82 (d, J=6.2 Hz, 3H). [M+H]⁺=794.6.

### Example 309: (R,E)-N-(1-(1-(4-((R)-2,6-dioxopiperidin-3-yl)-3,5-difluorophenyl)piperidin-4-yl)azetidin-3-yl)-N,1′,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

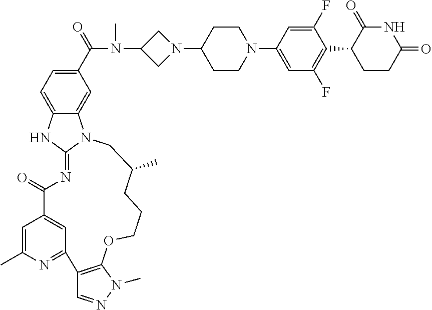 [SMILES: Cc1cc2cc(n1)-c1cnn(C)c1OCCC[C@@H](C)CN1/C(=N/C2=O)Nc2ccc(C(=O)N(C)C3CN(C4CCN(c5cc(F)c([C@H]6CCC(=O)NC6=O)c(F)c5)CC4)C3)cc21]

The titled compound was prepared in a manner similar to that in Example 260. ¹H NMR (500 MHz, DMSO) δ 10.85 (s, 1H), 8.43 (s, 1H), 7.93 (s, 1H), 7.66 (s, 1H), 7.57 (d, J=6.6 Hz, 2H), 7.23 (s, 1H), 6.57-6.55 (m, 2H), 4.41-4.30 (m, 1H), 4.20-4.18 (m, 1H), 4.07-3.94 (m, 3H), 3.73 (s, 3H), 3.53-3.51 (m, 2H), 3.12 (t, J=6.5 Hz, 2H), 3.04 (s, 2H), 2.92-2.71 (m, 5H), 2.56 (s, 3H), 2.29-2.15 (m, 2H), 2.11-1.87 (m, 4H), 1.68-1.57 (m, 1H), 1.51-1.40 (m, 1H), 1.32-1.12 (m, 7H), 0.87-0.77 (m, 4H); [M+H]⁺=849.7.

### Example 310: (R)-3-(2,6-difluoro-4-(2-(7-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)-2,7-diazaspiro nonan-2-yl)ethyl)phenyl)piperidine-2,6-dione

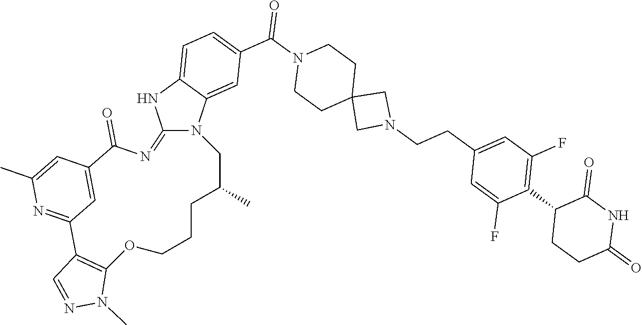 [SMILES: Cc1cc2cc(n1)-c1cnn(C)c1OCCC[C@@H](C)CN1/C(=N/C2=O)Nc2ccc(C(=O)N3CCC4(CC3)CN(CCc3cc(F)c([C@H]5CCC(=O)NC5=O)c(F)c3)C4)cc21]

The titled compound was prepared in a manner similar to that in Example 260. ¹H NMR (500 MHz, DMSO) δ 12.89 (s, 1H), 10.94 (s, 1H), 8.43 (s, 1H), 7.93 (s, 1H), 7.83 (s, 1H), 7.61-7.49 (m, 3H), 7.01 (d, J=10.2 Hz, 2H), 4.40-4.33 (m, 1H), 4.19 (dd, J=11.5, 6.8 Hz, 2H), 4.11-3.97 (m, 4H), 3.76 (s, 2H), 3.73 (s, 3H), 2.87-2.70 (m, 5H), 2.56 (s, 3H), 2.40-2.35 (m, 5H), 2.23 (d, J=6.8 Hz, 2H), 2.17-2.07 (m, 1H), 2.00-1.92 (m, 3H), 1.79-1.65 (m, 4H), 1.54-1.41 (m, 1H), 0.84 (t. J=12.6 Hz, 3H); [M+H]⁺=834.5.

### Example 312: (R)-3-(2,6-difluoro-4-(2-(3-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperidin-4-yl)azetidin-1-yl)ethyl)phenyl)piperidine-2,6-dione

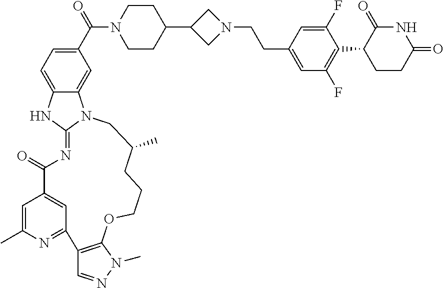 [SMILES: Cc1cc2cc(n1)-c1cnn(C)c1OCCC[C@@H](C)CN1/C(=N/C2=O)Nc2ccc(C(=O)N3CCC(C4CN(CCc5cc(F)c([C@H]6CCC(=O)NC6=O)c(F)c5)C4)CC3)cc21]

The titled compound was prepared in a manner similar to that in Example 260. ¹H NMR (500 MHz, DMSO) δ 12.85 (s, 1H), 10.94 (s, 1H), 8.42 (s, 1H), 7.92 (s, 1H), 7.65 (s, 1H), 7.56 (d, J=7.7 Hz, 2H), 7.23 (s, 1H), 6.98 (d, J=9.6 Hz, 2H), 4.39-4.31 (m, 1H), 4.19 (d, J=12.3 Hz, 2H), 4.09-3.97 (m, 2H), 3.73 (s, 3H), 2.89-2.75 (m, 6H), 2.66-2.60 (m, 4H), 2.56 (s, 6H), 2.31-2.08 (m, 4H), 2.03-1.88 (m, 4H), 1.73-1.59 (m, 2H), 1.52-1.37 (m, 2H), 1.08-0.97 (m, 2H), 0.82 (d, J=6.1 Hz, 3H); [M+H]⁺=848.6.

### Example 313: 1-(4-((R)-2,6-dioxopiperidin-3-yl)-3,5-difluorophenethyl)piperidin-4-yl (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxylate

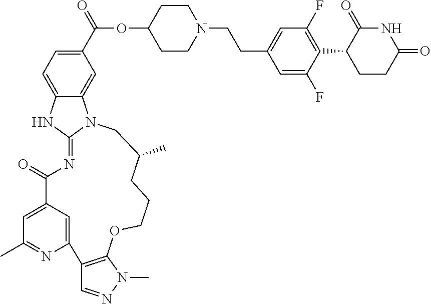 [SMILES: Cc1cc2cc(n1)-c1cnn(C)c1OCCC[C@@H](C)CN1/C(=N/C2=O)Nc2ccc(C(=O)OC3CCN(CCc4cc(F)c([C@H]5CCC(=O)NC5=O)c(F)c4)CC3)cc21]

The titled compound was prepared in a manner similar to that in Example 260. ¹H NMR (500 MHz, DMSO) δ 13.00 (s, 1H), 10.95 (s, 1H), 8.43 (s, 1H), 8.13 (d, J=9.8 Hz, 1H), 7.91 (d, J=10.7 Hz, 1H), 7.90 (s, 1H), 7.63 (d, J=8.3 Hz, 1H), 7.58 (s, 1H), 7.05 (d, J=10.1 Hz, 2H), 4.99 (s, 1H), 4.40-4.28 (m, 1H), 4.25-4.15 (m, 2H), 4.04 (s, 1H), 4.03-3.96 (m, 1H), 3.73 (s, 3H), 2.87-2.70 (m, 6H), 2.66-2.59 (m, 2H), 2.56 (s, 4H), 2.43-2.33 (m, 2H), 2.26-2.16 (m, 1H), 2.15-2.07 (m, 1H), 2.06-1.87 (m, 5H), 1.82-1.71 (m, 2H), 1.54-1.45 (m, 1H), 0.83 (d, J=6.4 Hz, 3H); [M+H]⁺=809.7.

### Example 314: (7R,E)-N-(1-(2-(1-(2,6-dioxopiperidin-3-yl)-3-methyl-2-oxo-2,3-dihydro-1H-benzo[d]imidazol-4-yl) ethyl)piperidin-4-yl)-N,1¹,2⁶,7-tetramethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carboxamide

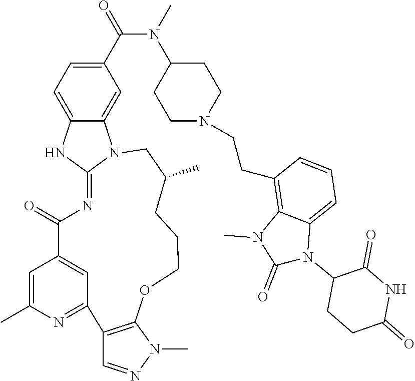 [SMILES: Cc1cc2cc(n1)-c1cnn(C)c1OCCC[C@@H](C)CN1/C(=N/C2=O)Nc2ccc(C(=O)N(C)C3CCN(CCc4cccc5c4n(C)c(=O)n5C4CCC(=O)NC4=O)CC3)cc21]

The titled compound was prepared in a manner similar to that in Example 260. ¹H NMR (500 MHz, DMSO) δ 12.82 (s, 1H), 11.08 (s, 1H), 8.43 (s, 1H), 7.95-7.89 (m, 1H), 7.67 (s, 1H), 7.60-7.53 (m, 2H), 7.23 (s, 1H), 6.95 (s, 2H), 5.34 (d, J=10.0 Hz, 1H), 4.36 (dd, J=11.0, 7.4 Hz, 1H), 4.20 (d, J=13.5 Hz, 2H), 4.05-4.02 (m, 4H), 3.73 (s, 3H), 3.64-3.49 (m, 6H), 2.93-2.87 (m, 6H), 2.63 (s, 4H), 2.56 (s, 3H), 2.21 (s, 2H), 2.02-1.91 (m, 3H), 1.83 (d, J=9.6 Hz, 2H), 1.69 (d, J=7.1 Hz, 2H), 1.44 (s, 1H), 0.82 (d, J=5.5 Hz, 3H); [M+H]⁺=856.5.

### Example 315: (7R,E)-N-(1-(2-(3-(2,6-dioxopiperidin-3-yl)-2-oxo-2,3-dihydrobenzo[d]oxazol-7-yl)ethyl)piperidin-4-yl)-N,1¹,2,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

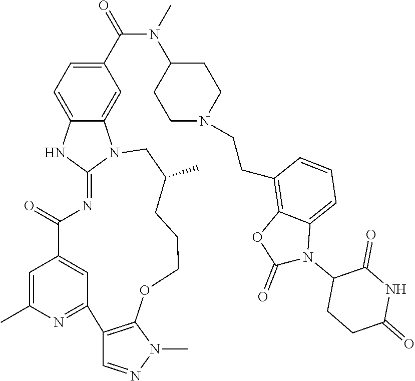 [SMILES: Cc1cc2cc(n1)-c1cnn(C)c1OCCC[C@@H](C)CN1/C(=N/C2=O)Nc2ccc(C(=O)N(C)C3CCN(CCc4cccc5c4oc(=O)n5C4CCC(=O)NC4=O)CC3)cc21]

The titled compound was prepared in a manner similar to that in Example 260. ¹H NMR (500 MHz, DMSO) δ 12.83 (s, 1H), 11.19 (d, J=1.4 Hz, 1H), 8.43 (s, 1H), 7.93 (s, 1H), 7.67 (s, 1H), 7.57 (d, J=7.3 Hz, 2H), 7.09-7.01 (m, 3H), 5.34 (d, J=7.7 Hz, 1H), 4.36 (d, J=4.7 Hz, 1H), 4.19 (d, J=11.8 Hz, 1H), 4.06-3.94 (m, 2H), 3.73 (s, 3H), 2.94-2.75 (m, 9H), 2.71-2.59 (m, 4H), 2.56 (s, 3H), 2.26-2.12 (m, 4H), 2.03-1.87 (m, 4H), 1.80 (d, J=9.3 Hz, 2H), 1.68 (d, J=10.1 Hz, 2H), 1.45 (d, J=6.7 Hz, 1H), 0.82 (d, J=6.2 Hz, 3H), [M+H]⁺=843.4.

### Example 316

(7R,E)-N-(1-(2-(1′-(2,6-dioxopiperidin-3-yl)-2′-oxospiro[cyclopropane-1,3′-indolin]-4′-yl)ethyl)piperidin-4-yl)-N,1¹,2⁶,7-tetramethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-carboxamide

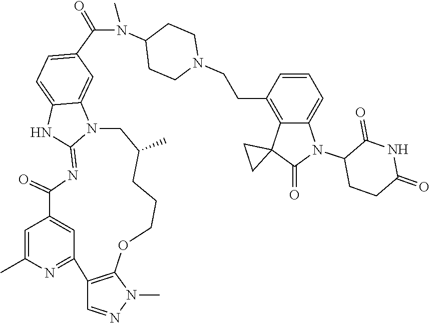 [SMILES: Cc1cc2cc(n1)-c1cnn(C)c1OCCC[C@@H](C)CN1/C(=N/C2=O)Nc2ccc(C(=O)N(C)C3CCN(CCc4cccc5c4C4(CC4)C(=O)N5C4CCC(=O)NC4=O)CC3)cc21]

The titled compound was prepared in a manner similar to that in Example 260.

¹H NMR (500 MHz, DMSO) δ 12.86 (s, 1H), 11.06 (s, 1H), 8.43 (s, 1H), 7.92 (d, J=2.3 Hz, 1H), 7.57 (d, J=7.2 Hz, 2H), 7.17 (d, J=51.8 Hz, 2H), 6.85 (s, 2H), 5.30 (s, 1H), 4.36 (s, 1H), 4.19 (d, J=13.2 Hz, 1H), 4.02 (d, J=15.4 Hz, 2H), 3.73 (d, J=2.4 Hz, 3H), 2.75-3.12 (m, 9H), 2.68-2.55 (m, 4H), 2.36 (s, 4H), 2.21 (s, 4H), 1.82 (d, J=11.1 Hz, 8H), 1.69 (s, 1H), 1.45 (s, 3H), 0.82 (d, J=6.4 Hz, 3H). [M+H]⁺=867.4

### Example 317: (7R,E)-N-(1-(2-(1-(2,6-dioxopiperidin-3-yl)-3,3-dimethyl-2-oxoindolin-4-yl)ethyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

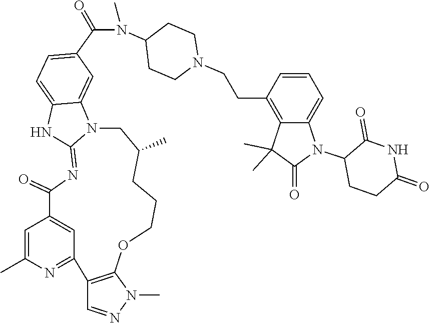 [SMILES: Cc1cc2cc(n1)-c1cnn(C)c1OCCC[C@@H](C)CN1/C(=N/C2=O)Nc2ccc(C(=O)N(C)C3CCN(CCc4cccc5c4C(C)(C)C(=O)N5C4CCC(=O)NC4=O)CC3)cc21]

The titled compound was prepared in a manner similar to that in Example 260.

¹H NMR (500 MHz, DMSO) δ 12.86 (s, 1H), 11.05 (s, 1H), 8.43 (s, 1H), 7.92 (s, 1H), 7.67 (s, 1H), 7.57 (d, J=6.2 Hz, 2H), 7.19 (d, J=41.5 Hz, 2H), 6.82 (s, 2H), 5.20 (s, 1H), 4.36 (q, J=8.2 Hz, 1H), 4.19 (d, J=13.3 Hz, 1H), 4.10-3.97 (m, 3H), 3.73 (s, 3H), 3.05-2.72 (m, 10H), 2.58 (d, J=15.1 Hz, 8H), 2.21 (s, 1H), 2.06-1.80 (m, 5H), 1.71 (s, 1H), 1.35 (d, J=20.9 Hz, 8H), 0.82 (d, J=6.5 Hz, 3H). [M+H]⁺=869.4

### Example 318: (R)-3-(2,6-difluoro-4-((R)-3-((R)-3-(methyl(((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)meth yl)amino)piperidine-1-carbonyl)pyrrolidin-1-yl)phenyl)piperidine-2,6-dione

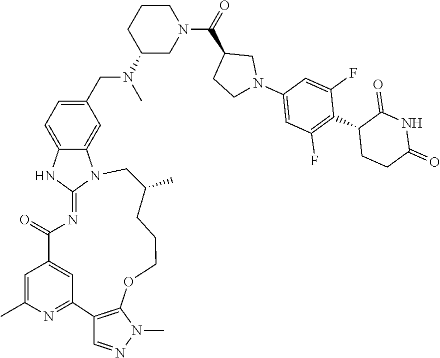 [SMILES: Cc1cc2cc(n1)-c1cnn(C)c1OCCC[C@@H](C)CN1/C(=N/C2=O)Nc2ccc(CN(C)[C@@H]3CCCN(C(=O)[C@@H]4CCN(c5cc(F)c([C@H]6CCC(=O)NC6=O)c(F)c5)C4)C3)cc21]

The titled compound was prepared in a manner similar to that in Example 5.

¹H NMR (500 MHz, DMSO) δ 12.63 (s, 1H), 10.77 (s, 1H), 8.34 (s, 1H), 7.85 (s, 1H), 7.50 (s, 1H), 7.44-7.39 (m, 2H), 7.17-7.09 (m, 1H), 6.16-6.10 (m, 2H), 4.36-4.28 (m, 2H), 4.13-4.10 (m, 1H), 3.96-3.79 (m, 5H), 3.65 (s, 3H), 3.47-3.34 (m, 4H), 3.20-3.11 (m, 2H), 3.06-2.98 (m, 1H), 2.80-2.64 (m, 3H), 2.48 (s, 3H), 2.41-2.31 (m, 1H), 2.23-2.10 (m, 5H), 2.02-1.87 (m, 7H), 1.73-1.65 (m, 1H), 1.60-1.47 (m, 1H), 1.39-1.31 (m, 2H), 0.73 (d, J=5.5 Hz, 3H): [M+H]⁺=877.7.

### Example 319: (R)-3-(2,6-difluoro-4-((R)-3-((S)-3-(methyl(((R,E)-1¹,2⁶,7-trimethyl-3-oxo-5²,5³-dihydro-1¹H,5′ H-11-oxa-4-aza-5(2,1)-benzo[d]imidazola-2(2,4)-pyridina-1(4,5)-pyrazolacycloundecaphane-5⁶-yl) methyl)amino)piperidine-1-carbonyl)pyrrolidin-1-yl)phenyl)piperidine-2,6-dione

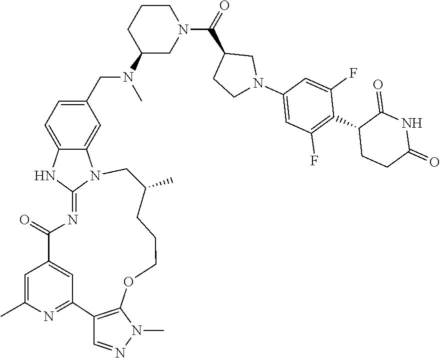 [SMILES: Cc1cc2cc(n1)-c1cnn(C)c1OCCC[C@@H](C)CN1/C(=N/C2=O)Nc2ccc(CN(C)[C@H]3CCCN(C(=O)[C@@H]4CCN(c5cc(F)c([C@H]6CCC(=O)NC6=O)c(F)c5)C4)C3)cc21]

The titled compound was prepared in a manner similar to that in Example 5.

¹H NMR (500 MHz, DMSO) δ 12.68 (s, 1H), 10.82 (s, 1H), 8.41 (s, 1H), 7.92 (s, 1H), 7.54-7.46 (m, 3H), 7.23-7.16 (m, 1H), 6.23-6.11 (m, 2H), 4.39-4.29 (m, 1H), 4.18-4.10 (m, 1H), 4.06-3.88 (m, 4H), 3.80-3.72 (m, 2H), 3.72 (s, 3H), 3.58-3.43 (m, 2H), 3.27-3.22 (m, 3H), 3.10-2.97 (m, 1H), 2.80-2.61 (m, 3H), 2.55 (s, 3H), 2.50-2.43 (m, 4H), 2.40-2.34 (m, 1H), 2.27-2.23 (m, 1H), 2.20-2.11 (m, 3H), 2.09-2.02 (m, 2H), 1.94-1.87 (m, 4H), 1.80-1.69 (m, 1H), 1.62-1.50 (m, 1H), 1.44-1.35 (m, 1H), 0.81 (d, J=5.5 Hz, 3H); [M+H]⁺=877.8.

### Example 320: (R)-3-(2,6-difluoro-4-(2-(4-((R,E)-1¹,26,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)phenyl)piperidine-2,6-dione

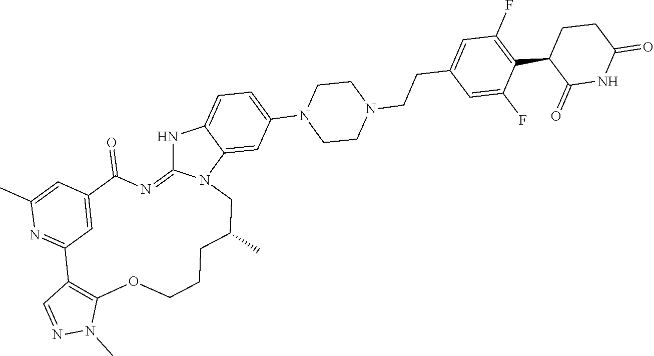 [SMILES: Cc1cc2cc(n1)-c1cnn(C)c1OCCC[C@@H](C)CN1/C(=N/C2=O)Nc2ccc(N3CCN(CCc4cc(F)c([C@H]5CCC(=O)NC5=O)c(F)c4)CC3)cc21]

The titled compound was prepared in a manner similar to that in Example 324.

¹H NMR (500 MHz, DMSO) δ 12.51 (s, 1H), 10.95 (s, 1H), 8.42 (s, 1H), 7.92 (s, 1H), 7.55 (s, 1H), 7.37 (d, J=8.6 Hz, 1H), 7.16 (s, 1H), 7.07 (d, J=10.1 Hz, 2H), 6.90 (d, J=8.9 Hz, 1H), 4.36-4.25 (m, 1H), 4.26-4.08 (m, 2H), 4.02-3.95 (m, 2H), 3.73 (s, 3H), 3.17 (s, 4H), 2.90-2.74 (m, 4H), 2.63 (s, 6H), 2.55 (s, 4H), 2.21 (s, 1H), 2.18-2.05 (m, 1H), 2.04-1.84 (m, 3H), 1.44 (s, 1H), 0.82 (d, J=6.4 Hz, 3H). [M+H]⁺=766.7

### Example 321: (R)-3-(2,6-difluoro-4-(3-((4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)methyl)azetidin-1-yl)phenyl)piperidine-2,6-dione

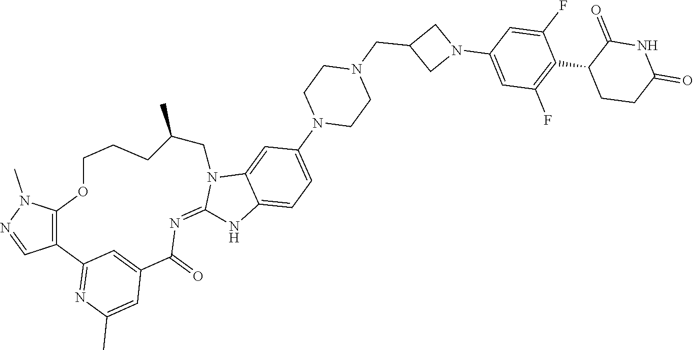 [SMILES: Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)CC3)cc21]

The titled compound was prepared in a manner similar to that in Example 324.

¹H NMR (500 MHz, DMSO) δ 12.51 (s, 1H), 10.85 (s, 1H), 8.42 (s, 1H), 7.92 (s, 1H), 7.56 (s, 1H), 7.37 (d, J=8.7 Hz, 1H), 7.15 (s, 1H), 6.89 (d, J=8.6 Hz, 1H), 6.12 (d, J=11.2 Hz, 2H), 4.42-3.38 (m, 1H), 4.14 (d, J=11.7 Hz, 1H), 4.07-3.89 (m, 5H), 3.73 (s, 3H), 3.52 (t, J=6.0 Hz, 2H), 3.29 (s, 1H), 3.16 (s, 4H), 3.04-2.90 (m, 1H), 2.87-2.72 (m, 2H), 2.64 (d, J=7.2 Hz, 2H), 2.59-2.52 (m, 7H), 2.22 (s, 1H), 2.08-1.83 (m, 1H), 1.94 (d, J=5.4 Hz, 3H), 1.44 (s, 1H), 0.82 (d, J=6.4 Hz, 3H). [M+H]⁺=807.6

### Example 322: (R)-3-(2,6-difluoro-4-(4-((4-((R,E)-1¹,2⁶,7-trimethyl-3-oxo-5²,5³-dihydro-1¹H,5¹H-11-oxa-4-aza-5(2, 1)-benzo[d]imidazola-2(2,4)-pyridina-1(4,5)-pyrazolacycloundecaphane-5⁶-yl)piperazin-1-yl)methyl)piperidin-1-yl)phenyl)piperidine-2,6-dione

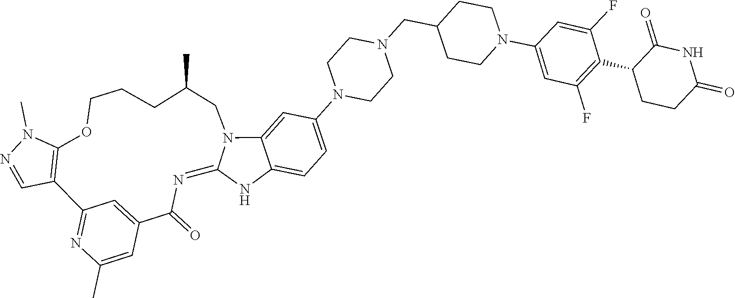 [SMILES: Cc1cc2cc(n1)-c1cnn(C)c1OCCC[C@@H](C)CN1/C(=N/C2=O)Nc2ccc(N3CCN(CC4CCN(c5cc(F)c([C@H]6CCC(=O)NC6=O)c(F)c5)CC4)CC3)cc21]

The titled compound was prepared in a manner similar to that in Example 324.

¹H NMR (500 MHz, DMSO) δ 12.51 (s, 1H), 10.87 (s, 1H), 8.42 (s, 1H), 7.92 (s, 1H), 7.56 (s, 1H), 7.37 (d, J=8.7 Hz, 1H), 7.15 (s, 1H), 6.90 (d, J=8.8 Hz, 1H), 6.62 (d, J=12.9 Hz, 2H), 4.36 (d, J=4.7 Hz, 1H), 4.14 (d, J=11.1 Hz, 1H), 3.99 (s, 3H), 3.85-3.66 (m, 5H), 3.17 (s, 4H), 2.79-2.72 (m, 4H), 2.57-2.52 (m, 7H), 2.23 (d, J=5.6 Hz, 3H), 2.14-2.03 (m, 1H), 1.97 (s, 3H), 1.79 (d, J=10.6 Hz, 3H), 1.43 (d, J=7.5 Hz, 1H), 1.18 (s, 3H), 0.82 (d, J=6.5 Hz, 3H). [M+H]⁺=835.8.

### Example 323: (R)-3-(2,6-difluoro-4-(4-((4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-1-yl)methyl) piperidin-1-yl)phenyl)piperidine-2,6-dione

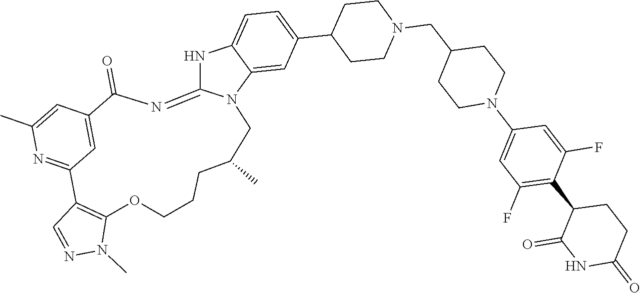 [SMILES: Cc1cc2cc(n1)-c1cnn(C)c1OCCC[C@@H](C)CN1/C(=N/C2=O)Nc2ccc(C3CCN(CC4CCN(c5cc(F)c([C@H]6CCC(=O)NC6=O)c(F)c5)CC4)CC3)cc21]

The titled compound was prepared in a manner similar to that in Example 1.

¹H NMR (500 MHz, DMSO) δ 12.64 (s, 1H), 10.87 (s, 1H), 8.43 (s, 1H), 7.92 (s, 1H), 7.55 (d, J=13.5 Hz, 2H), 7.44 (d, J=8.1 Hz, 1H), 7.13 (d, J=8.2 Hz, 1H), 6.62 (d, J=13.3 Hz, 2H), 4.36 (s, 1H), 4.17 (d, J=13.5 Hz, 1H), 4.09-3.93 (m, 3H), 3.73 (s, 3H), 2.98 (s, 2H), 2.77 (dt, J=24.2, 9.0 Hz, 4H), 2.56 (s, 3H), 2.20 (s, 3H), 1.98 (s, 6H), 1.78 (s, 8H), 1.46 (s, 1H), 1.24 (s, 3H), 1.16 (d, J=12.1 Hz, 1H), 0.82 (d, J=6.5 Hz, 4H). [M+H]⁺=834.4

### Example 324: (R)-3-(2,6-difluoro-4-(4-(4-((R,E)-1¹,26,7-trimethyl-3-oxo-5²,5³-dihydro-1¹H,5¹H-11-oxa-4-aza-5(2, 1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

### Step 1: tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylate

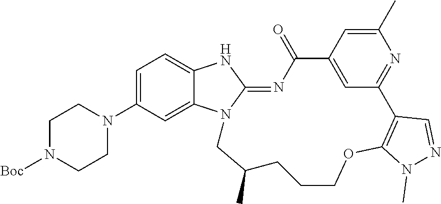 [SMILES: Cc1cc2cc(n1)-c1cnn(C)c1OCCC[C@@H](C)CN1/C(=N/C2=O)Nc2ccc(N3CCN(C(=O)OC(C)(C)C)CC3)cc21]

A mixture of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (1 g, 1.97 mmol), tert-butyl piperazine-1-carboxylate (549 mg, 2.95 mmol), Pd₂(dba)₃ (183 mg, 0.2 mmol), Ruphos (187 mg, 0.4 mmol), NaOBu (568 mg, 5.91 mmol) in DMA (20 mL) was stirred at 100° C. for 15 hrs. After cooling to rt, the reaction was quenched with sat. NH₄Cl solution (15 mL), and the resulting mixture was extracted with DCM (20 mL×3). The combined organic phase was washed with brine (20 mL), dried over Na₂SO₄, filtered and concentrated in vacuum. The residue was purified by silica gel column (DCM:CH₃OH=20:1) to afford tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylate (1.1 g, 90.9%). [M+H]⁺=615.5.

### Step 2: (R,E)-1¹,2⁶,7-trimethyl-5⁶-(piperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

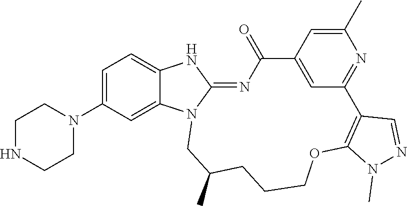 [SMILES: Cc1cc2cc(n1)-c1cnn(C)c1OCCC[C@@H](C)CN1/C(=N/C2=O)Nc2ccc(N3CCNCC3)cc21]

To a solution of tert-butyl (R,E)-4-(1¹,2⁶,7-trimethyl-3-oxo-5²,5³-dihydro-1¹H,5¹H-11-oxa-4-aza-5(2,1)-benzo[d]imidazola-2(2, 4)-pyridina-1(4,5)-pyrazolacycloundecaphane-5⁶-yl)piperazine-1-carboxylate (1 g, 1.63 mmol) in DCM (15 mL) was added TFA (3 mL). The reaction was stirred at rt for 2 hrs and then concentrated in vacuo. The residue was dissolved in DCM (30 mL), washed with sat. NaHCO₃ solution (20 mL) and brine (20 mL), dried over Na₂SO₄, filtered and concentrated in vacuum to afford (R,E)-1¹,2⁶,7-trimethyl-5⁶-(piperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (750 mg, 89.4%). [M+H]⁺=515.5.

### Step 3: (R)-3-(2,6-difluoro-4-(4-(4-((R,E)-1¹,2⁶,7-trimethyl-3-oxo-5²,5-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

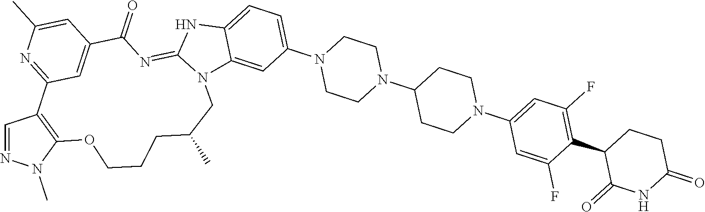 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21]

To a solution of (R,E)-1¹,2⁶,7-trimethyl-5⁶-(piperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 0.19 mmol), (R)-3-(2,6-difluoro-4-(4-oxopiperidin-1-yl)phenyl)piperidine-2,6-dione (81.3 mg, 0.25 mmol) in DCE (6 mL) was added STAB (121 mg, 0.57 mmol). Then the mixture was stirred at 50° C. for 1 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=94:6) to afford (R)-3-(2,6-difluoro-4-(4-(4-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione (31 mg, 19.9%). [M+H]⁺=821.7. ¹H NMR (500 MHz, DMSO) δ 12.51 (s, 1H), 10.87 (s, 1H), 8.42 (s, 1H), 7.92 (s, 1H), 7.55 (s, 1H), 7.37 (d, J=8.7 Hz, 1H), 7.14 (s, 1H), 6.89 (d, J=8.0 Hz, 1H), 6.63-6.61 (m, 2H), 4.36 (s, 1H), 4.14 (d, J=11.1 Hz, 1H), 4.07-4.03 (m, 1H), 4.01-3.90 (m, 2H), 3.82 (d, J=11.8 Hz, 2H), 3.73 (s, 3H), 3.16 (s, 4H), 2.87-2.73 (m, 4H), 2.69 (s, 4H), 2.55 (s, 5H), 2.21 (s, 1H), 2.09 (d, J=12.9 Hz, 1H), 2.01-1.81 (m, 5H), 1.61-1.39 (m, 3H), 0.82 (d, J=6.4 Hz, 3H). [M+H]⁺=821.6.

### Example 326: (R)-3-(4-((S)-3,3-dimethyl-4-(4-((R,E)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carbonyl)pyrrolidin-1-yl)-2,6-difluorophenyl)piperidine-2,6-dione

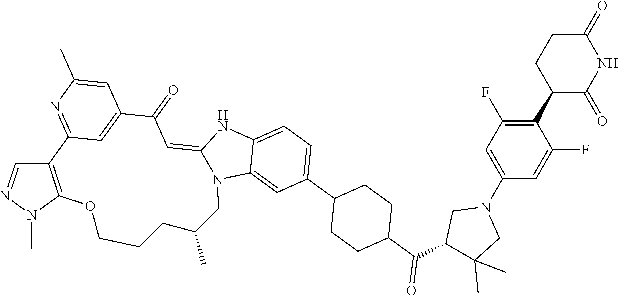 [SMILES: Cc1cc2cc(n1)-c1cnn(C)c1OCCC[C@@H](C)CN1/C(=C/C2=O)Nc2ccc(C3CCC(C(=O)[C@@H]4CN(c5cc(F)c([C@H]6CCC(=O)NC6=O)c(F)c5)CC4(C)C)CC3)cc21]

To a solution of (R,E)-1¹,2⁶,7-trimethyl-5⁶-(piperazin-1-yl)-5²,53-dihydro-1¹H,5¹H-11-oxa-4-aza-5(2,1)-benzo[d]imidazola-2(2,4)-pyridina-1(4,5)-pyrazolacycloundecaphan-3-one (50 mg, 0.097 mmol), (S)-1-(4-((R)-2,6-dioxopiperidin-3-yl)-3,5-difluorophenyl)-4,4-dimethylpyrrolidine-3-carboxylic acid (43 mg, 0.12 mmol) in DCM (5 mL) was added DIEA (25 mg, 0.19 mmol) and T3P (50% in EA, w/w) (120 mg, 0.19 mmol). Then the mixture was stirred at rt for 1 hr. H₂O (5 mL) was added and the resulting mixture was extracted with DCM (8 mL×3). The combined organic phase was washed with brine (8 mL), dried over Na₂SO₄, filtered and concentrated in vacuum. The residue was purified by silica gel column (DCM:CH₃OH=95:5) to afford (R)-3-(4-((S)-3,3-dimethyl-4-(4-((R,E)-1,2,7-trimethyl-3-oxo-5,5-dihydro-1H,5H-11-oxa-4-aza-5(2,1)-benzo[d]imidazola-2(2,4)-pyridina-1(4,5)-pyrazolacycloundecaphane-5-yl)piperazine-1-carbonyl) pyrrolidin-1-yl)-2,6-difluorophenyl)piperidine-2,6-dione (17 mg, 20.3%). [M+H]⁺=863.7. ¹H NMR (500 MHz, DMSO) δ 12.54 (s, 1H), 10.84 (s, 1H), 8.42 (s, 1H), 7.92 (s, 1H), 7.56 (s, 1H), 7.40 (d, J=8.6 Hz, 1H), 7.22 (s, 1H), 6.93 (d, J=9.0 Hz, 1H), 6.17 (d, J=12.3 Hz, 2H), 4.42-4.32 (m, 1H), 4.15 (d, J=12.1 Hz, 1H), 4.05-3.97 (m, 3H), 3.80 (s, 2H), 3.72 (d, J=11.0 Hz, 5H), 3.48 (s, 3H), 3.21 (s, 2H), 3.18-3.01 (m, 4H), 2.93-2.71 (m, 2H), 2.57-2.51 (m, 4H), 2.22 (s, 1H), 2.07 (s, 1H), 2.03-1.80 (m, 3H), 1.45 (s, 1H), 1.22 (s, 3H), 1.02 (s, 3H), 0.82 (d, J=6.3 Hz, 3H).

### Example 327: (R)-3-(4-((R)-3,3-dimethyl-4-(4-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carbonyl)pyrrolidin-1-yl)-2,6-difluorophenyl)piperidine-2,6-dione

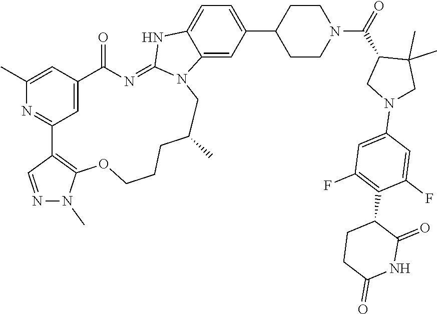

The titled compound was prepared in a manner similar to that in Example 158.

¹H NMR (500 MHz, DMSO-d₆) δ 12.65 (s, 1H), 10.84 (s, 1H), 8.43 (s, 1H), 7.92 (d, J=1.4 Hz, 1H), 7.58-7.49 (m, 2H), 7.46 (t, J=8.9 Hz, 1H), 7.22-7.05 (m, 1H), 6.17 (dd, J=12.5, 7.8 Hz, 2H), 4.67 (d, J=11.2 Hz, 1H), 4.36 (s, 1H), 4.1-4.32 (m, 3H),4.01 (dt, J=17.5, 8.1 Hz, 3H), 3.73 (s, 3H), 3.48 (dd, J=17.4, 7.1 Hz, 3H), 3.18 (dd, J=13.9, 8.7 Hz, 2H), 3.10 (d, J=3.6 Hz, 2H), 2.90 (d. J=11.2 Hz, 1H), 2.78 (dd, J=22.4, 9.4 Hz, 2H), 2.74-2.63 (m, 1H), 2.55 (s, 3H), 2.22 (s, 1H), 2.09 (dt, J=14.8, 11.2 Hz, 1H), 1.84 (d, J=15.1 Hz, 4H), 1.59 (s, 2H), 1.46 (s, 1H), 1.21 (d, J=9.4 Hz, 3H), 1.08 (s, 1H), 1.00 (s, 2H), 0.81 (d, J=6.4 Hz, 3H). [M+H]⁺=862.4

### Example 329: (R)-3-(2,6-difluoro-4-((R)-3-(4-((R,E)-5⁵-fluoro-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-1-carbonyl)pyrrolidin-1-yl)phenyl)piperidine-2,6-dione

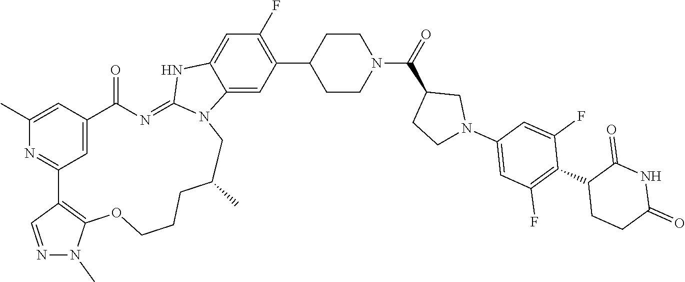

The titled compound was prepared in a manner similar to that in Example 158. ¹H NMR (500 MHz, DMSO) δ_{H} 12.72 (s, 1H), 10.84 (s, 1H), 8.42 (s, 1H), 7.92 (s, 1H), 7.69-7.58 (m, 1H), 7.56 (s, 1H), 7.30 (d, J=9.3 Hz, 1H), 6.24 (d, J=12.2 Hz, 2H), 4.63 (d, J=11.5 Hz, 1H), 4.35 (s, 1H), 4.16 (d. J=11.0 Hz, 2H), 4.02 (d, J=13.4 Hz, 3H), 3.73 (s, 3H), 3.59 (s, 1H), 3.47 (s, 2H), 3.24-3.15 (m, 2H), 2.87-2.66 (m, 4H), 2.57-2.53 (m, 5H), 2.26-2.05 (m, 4H), 2.02-1.63 (m, 7H), 1.45 (s, 1H), 0.81 (d, J=6.2 Hz, 3H). [M+H]⁺=852.6.

### Example 330: (R)-3-(2,6-difluoro-4-(2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-1-yl)ethyl)phenyl)piperidine-2,6-dione

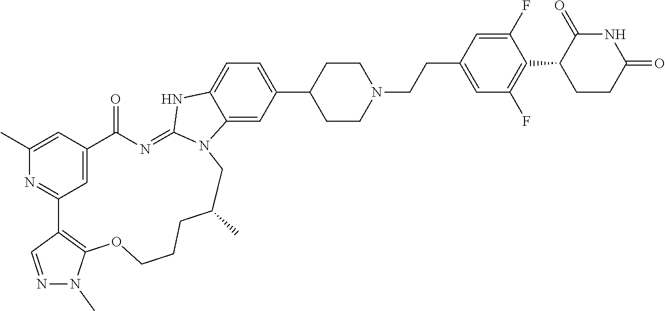 [SMILES: Cc1cc2cc(n1)-c1cnn(C)c1OCCC[C@@H](C)CN1/C(=N/C2=O)Nc2ccc(C3CCN(CCc4cc(F)c([C@H]5CCC(=O)NC5=O)c(F)c4)CC3)cc21]

The titled compound was prepared in a manner similar to that in Example 1.

¹H NMR (500 MHz, DMSO-d₆) δ 12.65 (s, 1H), 10.96 (s, 1H), 8.43 (s, 1H), 7.92 (s, 1H), 7.55 (d, J=15.8 Hz, 2H), 7.45 (d, J=8.1 Hz, 1H), 7.13 (d, J=8.2 Hz, 1H), 7.07 (d, J=10.4 Hz, 2H), 4.36 (d, J=4.8 Hz, 1H), 4.29-4.12 (m, 2H), 4.00 (t, J=6.8 Hz, 2H), 3.73 (s, 3H), 3.16 (s, 2H), 2.89-2.76 (m, 6H), 2.56 (m, 5H), 2.27-2.07 (m, 3H), 2.07-1.90 (m, 4H), 1.83 (s, 4H), 1.45 (s, 1H), 0.82 (d, J=6.5 Hz, 3H). [M+H]⁺=765.4

### Example 331: (R)-3-(2,6-difluoro-4-(3-((4-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5-yl)piperidin-1-yl)methyl) azetidin-1-yl)phenyl)piperidine-2,6-dione

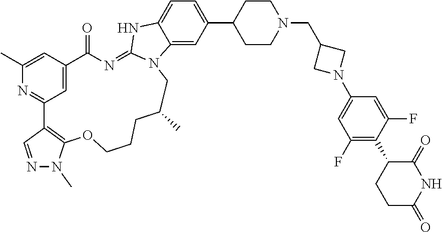 [SMILES: Cc1cc2cc(n1)-c1cnn(C)c1OCCC[C@@H](C)CN1/C(=N/C2=O)Nc2ccc(C3CCN(CC4CN(c5cc(F)c([C@H]6CCC(=O)NC6=O)c(F)c5)C4)CC3)cc21]

The titled compound was prepared in a manner similar to that in Example 1.

¹H NMR (500 MHz, DMSO) δ 12.64 (s, 1H), 10.86 (s, 1H), 8.43 (s, 1H), 7.92 (s, 1H), 7.55 (d, J=13.9 Hz, 2H), 7.44 (d, J=8.1 Hz, 1H), 7.12 (d, J=8.2 Hz, 1H), 6.12 (d, J=11.6 Hz, 2H), 4.36 (dq, J=9.5, 4.5 Hz, 1H), 4.17 (d, J=13.1 Hz, 1H), 4.10-3.99 (m, 3H), 3.95 (t, J=7.9 Hz, 3H), 3.73 (s, 3H), 3.50 (t, J=6.6 Hz, 2H), 2.98 (d, J=10.7 Hz, 3H), 2.78 (ddd, J=17.4, 13.3, 5.5 Hz, 3H), 2.62 (d, J=7.5 Hz, 2H), 2.55 (s, 3H), 2.22 (s, 1H), 2.08 (h, J=9.0, 8.4 Hz, 3H), 2.02-1.88 (m, 3H), 1.84-1.69 (m, 4H), 1.45 (s, 1H), 0.82 (d, J=6.4 Hz, 3H). [M+H]⁺=806.4

### Example 332: (R)-3-(2,6-difluoro-4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-[1,4′-bipiperidin]-1′-yl)phenyl)piperidine-2,6-dione

### Step 1: 8-(4-(2,6-bis(benzyloxy)pyridin-3-yl)-3,5-difluorophenyl)-1,4-dioxa-8-azaspiro[4.5]decane

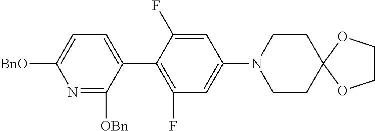 [SMILES: Fc1cc(N2CCC3(CC2)OCCO3)cc(F)c1-c1ccc(OCc2ccccc2)nc1OCc1ccccc1]

To the solution of 2,6-bis(benzyloxy)-3-(4-bromo-2,6-difluorophenyl)pyridine (30 g, 62.24 mmol), 1,4-dioxa-8-azaspiro[4.5]decane (10.68 g, 74.69 mmol) and Cs₂CO₃ (40.58 g, 124.48 mmol) in 500 mL dioxane, Pd₂(dba)₃ (2.85 g, 3.11 mmol) and Xantphos (3.6 g, 6.22 mmol) was added at N₂ atmosphere. The mixture was stirred at 80° C. for 16 hours under N₂ protected. The mixture was diluted with EtOAc and filtered. The filtrated was concentrated in vacuum and purified by silica column chromatography (EA:PE=0-80%) to afford the crude product. The crude was recrystallized with MeOH and filtered. The filter cake was dried to afford the title compound (26.8 g, 79% yield); [M+H]⁺=544.9.

### Step 2: 3-(2,6-difluoro-4-(1,4-dioxa-8-azaspiro[4.5]decan-8-yl)phenyl)piperidine-2,6-dione

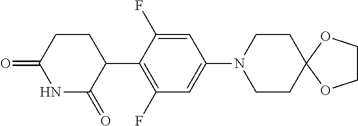 [SMILES: O=C1CCC(c2c(F)cc(N3CCC4(CC3)OCCO4)cc2F)C(=O)N1]

To the solution of 8-(4-(2,6-bis(benzyloxy)pyridin-3-yl)-3,5-difluorophenyl)-1,4-dioxa-8-azaspiro[4.5]decane (26.8 g, 49.26 mmol) in 400 mL DMF and 80 mL iPrOH, Pd/C (27 g, 10 wt. %, wet) was added. The mixture was stirred at 45° C. for 16 hours at H₂ atmosphere (4 bar). The mixture was filtered and the filter cake was washed with DMF. The combined liquid was concentrated in vacuum to afford the title compound (15 g, 83.2% yield); [M+H]⁺=367.1.

### Step 3: (R)-3-(2,6-difluoro-4-(1,4-dioxa-8-azaspiro[4.5]decan-8-yl)phenyl)piperidine-2,6-dione

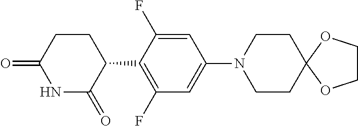 [SMILES: O=C1CC[C@H](c2c(F)cc(N3CCC4(CC3)OCCO4)cc2F)C(=O)N1]

The title compound was purified by SFC (IF (2*25 cm, 5 um), MtBE (0.1° % DEA) (MeOH DCM=:1)=50:50, 100 bar, 20 ml/min) and the title compound corresponded to peak A @ 0.990 min/254 nm (4.47 g, 37% yield from 12 g racemate); [M+H]⁺=367.1.

### Step 4: (R)-3-(2,6-difluoro-4-(4-oxopiperidin-1-yl)phenyl)piperidine-2,6-dione

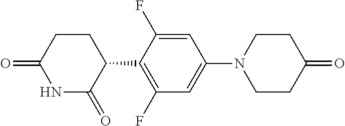 [SMILES: O=C1CCN(c2cc(F)c([C@H]3CCC(=O)NC3=O)c(F)c2)CC1]

(R)-3-(2,6-difluoro-4-(1,4-dioxa-8-azaspiro[4.5]decan-8-yl)phenyl)piperidine-2,6-dione (1 g, 2.73 mmol) was placed in 100 mL round bottom flask with a magnetic stir bar. Then, 10 mL 12N HCl aqueous was added. The mixture was stirred at room temperature for 30 minutes. The mixture was added dropwise to sat. aq. NaHCO₃ solution and finally pH=6-7. The liquid was extracted with DCM and separated. The organic phase was concentrated in vacuum and purified with combiflash (MeOH:DCM=0-5%) to afford the title compound (850 mg, 96.7% yield); [M+H]⁺=323.1.

### Step 5: (R)-3-(2,6-difluoro-4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-[1,4′-bipiperidin]-1′-yl)phenyl)piperidine-2,6-dione

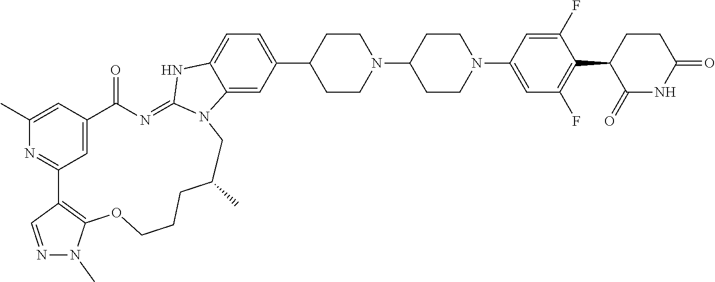 [SMILES: Cc1cc2cc(n1)-c1cnn(C)c1OCCC[C@@H](C)CN1/C(=N/C2=O)Nc2ccc(C3CCN(C4CCN(c5cc(F)c([C@H]6CCC(=O)NC6=O)c(F)c5)CC4)CC3)cc21]

The titled compound was prepared in a manner similar to that in Example 1.

¹H NMR (500 MHz, DMSO) δ 12.63 (s, 1H), 10.87 (s, 1H), 8.43 (s, 1H), 7.92 (s, 1H), 7.55 (d, J=15.3 Hz, 2H), 7.43 (d, J=8.1 Hz, 1H), 7.11 (d, J=8.2 Hz, 1H), 6.64 (d, J=13.0 Hz, 2H), 4.36 (dd, J=10.2, 5.7 Hz, 1H), 4.17 (d, J=13.4 Hz, 1H), 4.11-3.95 (m, 3H), 3.82 (d, J=12.2 Hz, 2H), 3.73 (s, 3H), 3.02 (d, J=10.4 Hz, 2H), 2.77 (q, J=12.7, 11.7 Hz, 4H), 2.52-2.55 (m, 6H), 2.26 (t, J=11.6 Hz, 3H), 2.09 (td, J=13.9, 9.9 Hz, 1H), 2.03-1.88 (m, 3H), 1.77 (dq, J=30.2, 13.5 Hz, 6H), 1.50 (dd, J=19.4, 10.1 Hz, 3H), 0.81 (d, J=6.5 Hz, 3H). [M+H]⁺=820.4

### Example 333: (R)-3-(2,6-difluoro-4-(4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperazin-1-yl)phenyl)piperidine-2,6-dione

### Step 1: tert-butyl (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carboxylate

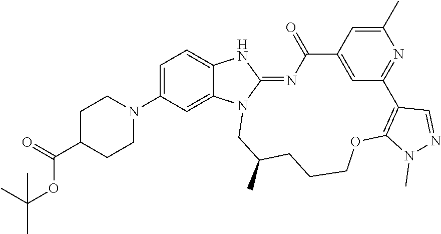 [SMILES: Cc1cc2cc(n1)-c1cnn(C)c1OCCC[C@@H](C)CN1/C(=N/C2=O)Nc2ccc(N3CCC(C(=O)OC(C)(C)C)CC3)cc21]

A mixture of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (3.5 g, 6.87 mmol), tert-butyl piperidine-4-carboxylate (2.54 g, 13.74 mmol), Pd(OAc)₂ (308.5 mg, 1.37 mmol), XPhos (1.31 g, 2.74 mmol), tBuONa (42.64 g, 27.4 mmol) in DMA (5 mL) was stirred at 110° C. under nitrogen atmosphere for 3 hrs. After cooling to rt. The reaction was quenched with NH₄Cl (aq.) (200 mL) and extracted with DCM (3×80 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to afford product (2.1 g, 50%) [M+H]⁺=614.5

### Step 2: (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carboxylic acid

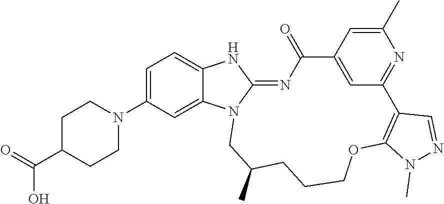 [SMILES: Cc1cc2cc(n1)-c1cnn(C)c1OCCC[C@@H](C)CN1/C(=N/C2=O)Nc2ccc(N3CCC(C(=O)O)CC3)cc21]

Tert-butyl (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carboxylate (2.1 g, 3.42 mmol) was dissolved in DCM:TFA=1:1(50 mL). The resulting solution was stirred for 2 h at room temperature. The reaction solution was evaporated to dryness. The crude residue was suspended with NaHCO₃(aq.) (50 mL) and extracted with DCM:MeOH=10:1 (3×80 mL). The combined organic layers were dried over anhydrous Na₂SO₄, then evaporated to dryness to afford the product (1.8 g, 94.5%). [M+H]⁺=558.5

### Step 3: benzyl 4-(4-(2,6-bis(benzyloxy)pyridin-3-yl)-3,5-difluorophenyl)piperazine-1-carboxylate

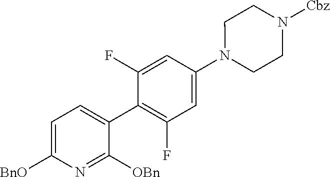 [SMILES: O=C(OCc1ccccc1)N1CCN(c2cc(F)c(-c3ccc(OCc4ccccc4)nc3OCc3ccccc3)c(F)c2)CC1]

To the solution of 2,6-bis(benzyloxy)-3-(4-bromo-2,6-difluorophenyl)pyridine (4.8 g, 10 mmol) (made by the same way described in WO2022012623 A1), benzyl piperazine-1-carboxylate (2.4 g, 11 mmol) and CS₂CO₃ (6.5 g, 20 mmol) in 100 mL dioxane was added Pd₂(dba)₃ (457 mg, 0.5 mmol) and Xantphos (578 mg, 1 mmol). The mixture was stirred at 90° C. for 18 hours under N₂ atmosphere. After LCMS showed the reaction was completed, the mixture was diluted with EtOAc and filtered. The filtrate was concentrated in vacuum and the crude product was purified by silica column chromatography (EA:PE=0-50%) to afford the desired product (5.9 g, 95%). [M+H]⁺=622.7.

### Step 4: (R)-3-(2,6-difluoro-4-(piperazin-1-yl)phenyl)piperidine-2,6-dione

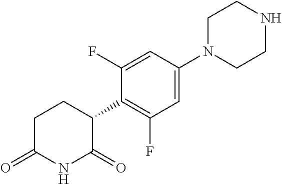 [SMILES: O=C1CC[C@H](c2c(F)cc(N3CCNCC3)cc2F)C(=O)N1]

To the solution of benzyl 4-(4-(2,6-bis(benzyloxy)pyridin-3-yl)-3,5-difluorophenyl)piperazine-1-carboxylate (5.9 g, 9.5 mmol) in 5 mL DCM and 100 mL IPA was added Pd/C (5.9 g, 10% w.t.). The mixture was stirred at 45° C. for 18 hours under H₂ atmosphere. After LCMS showed the reaction was completed, the mixture was filtered through celite. The filter cake was suspended in 50 mL DMF and filtered. The filtrate was combined and concentrated in vacuum. The crude product was washed with MeOH and purified by HPLC (IF (2*25 cm, Sum), 60% MtBE/40% MeOH:DCM=1:1, 80 bar, 20 ml/min) and corresponded to peak A @3.196 min/254 nm (2.5 g, 85%). [M+H]⁺=310.7.

### Step 5: (R)-3-(2,6-difluoro-4-(4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperazin-1-yl)phenyl)piperidine-2,6-dione

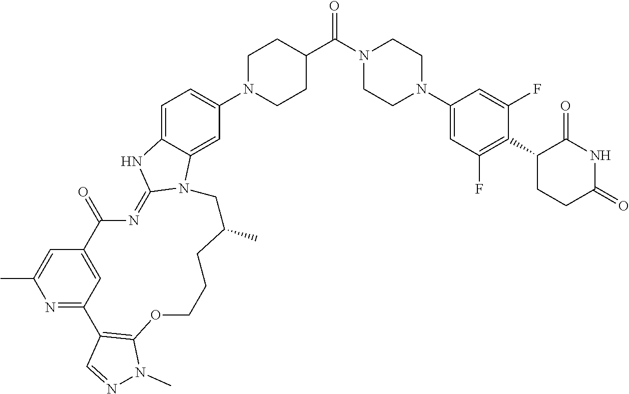 [SMILES: Cc1cc2cc(n1)-c1cnn(C)c1OCCC[C@@H](C)CN1/C(=N/C2=O)Nc2ccc(N3CCC(C(=O)N4CCN(c5cc(F)c([C@H]6CCC(=O)NC6=O)c(F)c5)CC4)CC3)cc21]

To a solution of (R)-3-(2,6-difluoro-4-(piperazin-1-yl)phenyl)piperidine-2,6-dione (1.23 g, 3.55 mmol), (R,E)-1-(1¹,2⁶,7-trimethyl-3-oxo-5²,5^{t}-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carboxylic acid (1.8 g, 3.23 mmol), and DIEA (1.25 g, 9.68 mmol) in DCM (30 mL) was added T3P (2.57 g, 8.06 mmol, 50 wt %) dropwise at room temperature. The resulting mixture was stirred at room temperature for 0.5 hour. The reaction was quenched with water (80 mL) and extracted with DCM (2×50 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC (C-18 column chromatography (0.1% FA in water:acetonitrile=90: 10-60:40 gradient elution) to give the desired product (2 g, 72.9%). ¹H NMR (500 MHz, DMSO) δ 12.51 (s, 1H), 10.88 (s, 1H), 8.42 (s, 1H), 7.92 (s, 1H), 7.56 (s, 1H), 7.37 (d, J=8.7 Hz, 1H), 7.16 (s, 1H), 6.95-6.86 (m, 1H), 6.69 (d, J=13.1 Hz, 2H), 4.46-4.30 (m, 1H), 4.14 (d, J=13.2 Hz, 1H), 4.08 (dd, J=12.6, 5.0 Hz, 1H), 4.00 (d, J=10.5 Hz, 2H), 3.71 (d, J=23.4 Hz, 6H), 3.59 (s, 2H), 3.24 (d, J=34.3 Hz, 5H), 2.91-2.74 (m, 5H), 2.55 (s, 3H), 2.22 (s, 1H), 2.08 (dd, J=14.4, 10.6 Hz, 1H), 2.04-1.88 (m, 3H), 1.76 (d, J=8.1 Hz, 4H), 1.45 (s, 1H), 1.23 (s, 1H), 0.82 (d, J=6.5 Hz, 3H). [M+H]⁺=849.5.

### Example 335: N-(4-((R)-2,6-dioxopiperidin-3-yl)-3,5-difluorophenethyl)-N-methyl-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carboxamide

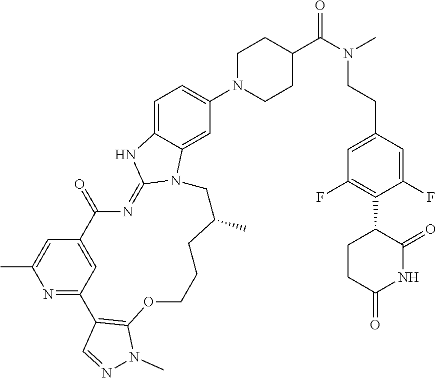 [SMILES: Cc1cc2cc(n1)-c1cnn(C)c1OCCC[C@@H](C)CN1/C(=N/C2=O)Nc2ccc(N3CCC(C(=O)N(C)CCc4cc(F)c([C@H]5CCC(=O)NC5=O)c(F)c4)CC3)cc21]

The titled compound was prepared in a manner similar to that in Example 333.

¹H NMR (500 MHz, DMSO) δ 12.86 (s, 1H) δ 7.73 (s, 1H), 7.27 (s, 1H), 6.86 (s, 1H), 6.57 (s, 1H), 6.19 (m, 5H), 3.69 (s, 1H), 3.47 (d, J=11.2 Hz, 2H), 3.21 (s, 1H), 2.98 (d, J=2.8 Hz, 4H), 2.85 (s, 2H), 2.30 (s, 1H), 2.22 (s, 1H), 2.18 (s, 1H), 2.07 (d, J=21.2 Hz, 2H), 2.02 (s, 2H), 1.81 (s, 4H), 1.50 (s, 3H), 1.25 (d, J=12.1 Hz, 4H), 1.01 (s, 3H), 0.74 (s, 1H), 0.64 (s, 1H), 0.50 (s, 2H), 0.09 (s, 4H). [M+H]⁺=822.5

### Example 336: (R,E)-N-(1-(4-((R)-2,6-dioxopiperidin-3-yl)-3,5-difluorophenethyl)piperidin-4-yl)-N,1¹,2⁶,5⁵,7-penta methyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

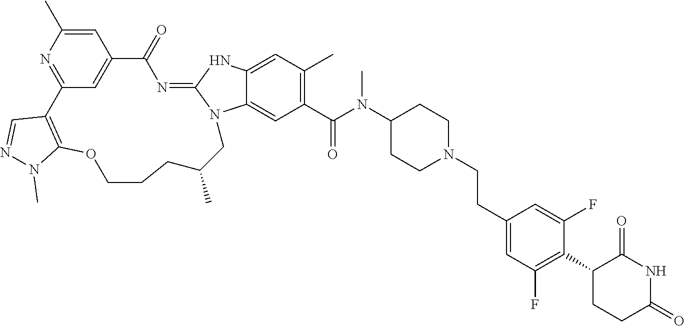 [SMILES: Cc1cc2cc(n1)-c1cnn(C)c1OCCC[C@@H](C)CN1/C(=N/C2=O)Nc2cc(C)c(C(=O)N(C)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.74 (d, J=7.4 Hz, 1H), 10.93 (d, J=15.2 Hz 1¹H), 8.42 (s, 1H), 7.92 (s, 1H), 7.60-7.34 (m, 3H), 7.05 (d, J=10.4 Hz, 1H), 6.96-6.92 (m, 1H), 4.36 (s, 2H), 4.23-4.18 (m, 2H), 4.06-3.96 (m, 2H), 3.73 (s, 3H), 3.07 (s, 2H), 2.92 (s, 3H), 2.83-2.75 (m, 4H), 2.65-2.61 (m, 3H), 2.56 (s, 3H), 2.38-2.34 (m, 1H), 2.27-2.23 (m, 3H), 2.17-2.13 (m, 2H), 1.99-1.84 (m, 3H), 1.83-1.78 (m, 2H), 1.72-1.63 (m, 2H), 1.51-1.38 (m, 2H), 0.82-0.73 (m, 3H). [M+H]⁺=836.6.

### Example 337: (R,E)-N—((S)-1-(4-((R)-2,6-dioxopiperidin-3-yl)-3,5-difluorophenethyl)-3,3-difluoropiperidin-4-yl)-1¹2⁶, 7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

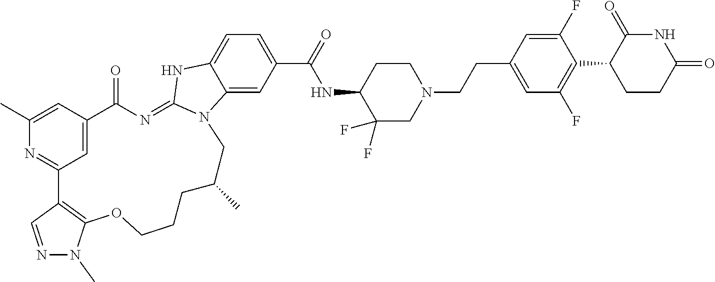 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)CC3(F)F)cc21]

The titled compound was prepared in a manner similar to that in Example 260.

¹H NMR (500 MHz, DMSO) δ 12.92 (s, 1H), 10.95 (s, 1H), 8.43 (d, J=7.6 Hz, 2H), 8.17 (s, 1H), 7.93 (s, 1H), 7.86 (d, J=8.2 Hz, 1H), 7.57 (d, J=8.0 Hz, 2H), 7.06 (d, J=10.2 Hz, 2H), 4.54-4.42 (m, 1H), 4.39-4.32 (m, 1H), 4.26-4.17(m, 2H), 4.02-3.92 (m, 2H), 3.74 (s, 3H), 3.03-2.96 (m, 1H), 2.91-2.84 (m, 1H), 2.83-2.75 (m, 3H), 2.71-2.66 (m, 2H), 2.60-2.52 (m, 4H), 2.48-2.40 (m, 2H), 2.32-2.26 (m, 1H), 2.26-2.07 (m, 2H), 2.02 (s, 2H), 1.96-1.76 (m, 3H), 1.48 (s, 1H), 0.84 (d, J=6.3 Hz, 3H). [M+H]⁺=844.8

### Example 339: (R,E)-N-(4-(4-((R)-2,6-dioxopiperidin-3-yl)-3,5-difluorophenyl)butyl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

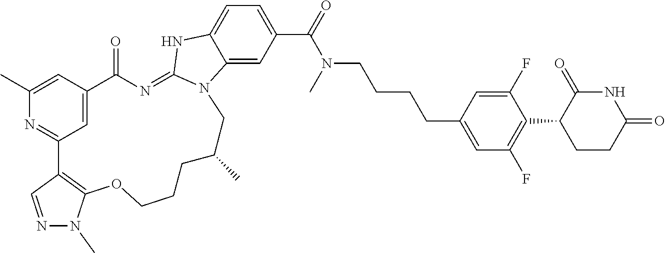 [SMILES: Cc1cc2cc(n1)-c1cnn(C)c1OCCC[C@@H](C)CN1/C(=N/C2=O)Nc2ccc(C(=O)N(C)CCCCc3cc(F)c([C@H]4CCC(=O)NC4=O)c(F)c3)cc21]

The titled compound was prepared in a manner similar to that in Example 96.

¹H NMR (500 MHz, DMSO) δ 12.85 (s, 1H), 10.93 (s, 1H), 8.43 (s, 1H), 7.92 (s, 1H), 7.66 (s, 1H), 7.57 (s, 1H), 7.56 (d, J=9.5 Hz, 1H), 7.26-7.20 (m, 1H), 7.02-6.86 (m, 2H), 4.40-4.32 (m, 1H), 4.19-4.17 (m, 2H), 4.06-3.94 (m, 2H), 3.73 (s, 3H), 3.55-3.44 (m, 1H), 3.01-2.88 (m, 4H), 2.82-2.77 (m, 3H), 2.56 (s, 3H), 2.26-2.07 (m, 3H), 2.00-1.89 (m, 3H), 1.71-1.34 (m, 6H), 0.80 (d, J=5.5 Hz, 3H); [M+H]⁺=767.8.

### Example 340: (R,E)-N—((S)-1-(4-((R)-2,6-dioxopiperidin-3-yl)-3,5-difluorophenethyl)azepan-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

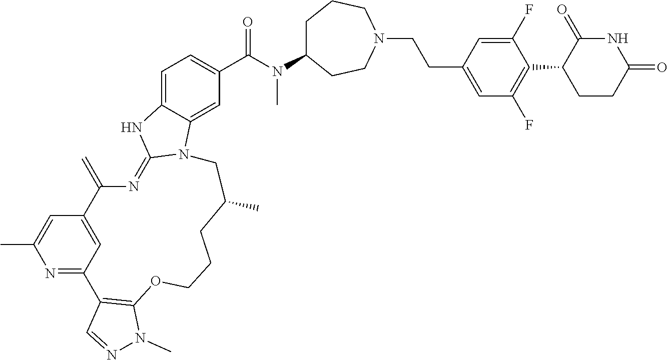 [SMILES: C=C1/N=C2\Nc3ccc(C(=O)N(C)[C@H]4CCCN(CCc5cc(F)c([C@H]6CCC(=O)NC6=O)c(F)c5)CC4)cc3N2C[C@H](C)CCCOc2c(cnn2C)-c2cc1cc(C)n2]

The titled compound was prepared in a manner similar to that in Example 260.

¹H NMR (500 MHz, DMSO) δ 12.84 (s, 1H), 10.91 (s, 1H), 8.43 (s, 1H), 7.92 (s, 1¹H), 7.67-7.63 (m, 1H), 7.57 (s, 1H), 7.56 (d, J=10.5 Hz, 1H), 7.23-7.19 (m, 1H), 7.06-6.89 (m, 2H), 4.40-4.32 (m, 1H), 4.20-4.10 (m, 2H), 4.03-3.95 (m, 2H), 3.73 (s, 3H), 2.92-2.68 (m, 9H), 2.56 (s, 3H), 2.50-2.36 (m, 7H), 2.27-2.16 (m, 1H), 2.13-1.90 (m, 4H), 1.84-1.70 (m, 4H), 1.66-1.56 (m, 1H), 1.50-1.40 (m, 1H), 0.82 (d, J=6.0 Hz, 3H); [M+H]⁺=836.9.

### Example 341: (R)-3-(2,6-difluoro-4-(3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carbonyl)piperazin-1-yl)propyl)phenyl)piperidine-2,6-dione

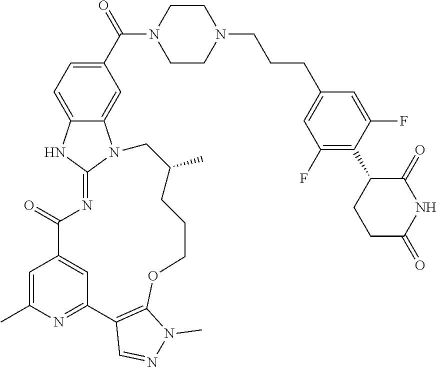 [SMILES: Cc1cc2cc(n1)-c1cnn(C)c1OCCC[C@@H](C)CN1/C(=N/C2=O)Nc2ccc(C(=O)N3CCN(CCCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.85 (s, 1H), 10.94 (s, 1H), 8.43 (s, 1H), 7.92 (s, 1H), 7.67 (s, 1H), 7.57 (d, J=6.4 Hz, 2H), 7.25 (d, J=8.0 Hz, 1H), 7.00 (d, J=10.0 Hz, 2H), 4.36 (d, J=4.5 Hz, 1H), 4.19 (d, J=13.3 Hz, 2H), 4.07-3.94 (m, 2H), 3.73 (s, 3H), 3.62 (s, 2H), 3.35 (s, 2H), 2.86-2.76 (m, 2H), 2.65-2.60 (m, 2H), 2.56 (s, 3H), 2.54 (s, 1H), 2.46-2.29 (m, 6H), 2.21 (s, 1H), 2.17-2.06 (m, 1H), 2.08-1.91 (m, 3H), 1.81-1.70 (m, 2H), 1.45 (s, 1H), 0.82 (d, J=6.4 Hz, 3H). [M+H]⁺=808.7

### Example 342: (R,E)-N—((3S,4S)-1-(4-((R)-2,6-dioxopiperidin-3-yl)-3,5-difluorophenethyl)-3-fluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

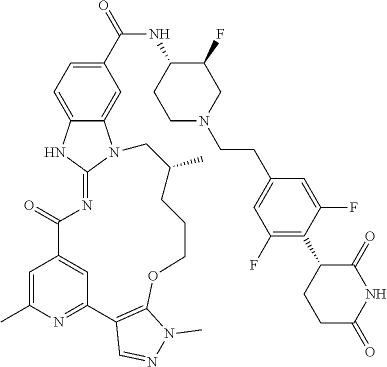 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)C[C@@H]3F)cc21]

The titled compound was prepared in a manner similar to that in Example 260.

¹H NMR (500 MHz, DMSO) δ 12.91 (s, 1H), 10.95 (s, 1H), 8.44 (d, J=6.8 Hz, 2H), 8.05 (s, 1H), 7.93 (d, J=6.4 Hz, 1H), 7.78 (d, J=7.6 Hz, 1H), 7.57 (t, .=7.1 Hz, 2H), 7.14-6.95 (m, 2H), 4.73-4.48 (m, 1H), 4.36 (d, J=3.8 Hz, 1H), 4.22 (d, J=3.9 Hz, 2H), 4.10-3.94 (m, 3H), 3.74 (d, J=6.4 Hz, 3H), 2.94-2.78 (m, 5H), 2.68 (s, 2H), 2.56 (s, 4H), 2.26-1.85 (m, 9H), 1.68-1.41 (m, 2H), 0.84 (d, J=6.1 Hz, 3H). [M+H]⁺=826.7

### Example 343: (R,E)-N—((3R,4S)-1-(4-((R)-2,6-dioxopiperidin-3-yl)-3,5-difluorophenethyl)-3-fluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

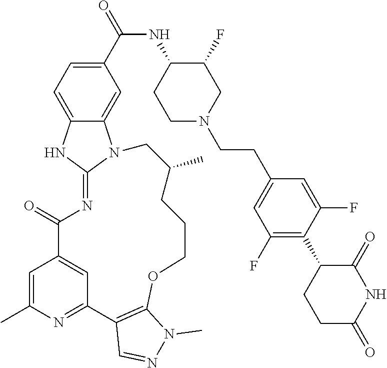 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)C[C@H]3F)cc21]

The titled compound was prepared in a manner similar to that in Example 260.

¹H NMR (500 MHz, DMSO) δ 12.90 (s, 1H), 10.95 (s, 1H), 8.44 (s, 1H), 8.37 (d, J=7.6 Hz, 1H), 8.17 (s, 1H), 7.93 (s, 1H), 7.85 (d, J=8.5 Hz, 1H), 7.61-7.51 (m, 2H), 7.06 (d, J=10.2 Hz, 2H), 4.90-4.74 (m, 1H), 4.37 (d, J=4.4 Hz, 1H), 4.27-4.15 (m, 2H), 4.14-3.92 (m, 3H), 3.74 (s, 3H), 3.02 (d, J=10.2 Hz, 1H), 2.90-2.76 (m, 4H), 2.61 (t, J=7.2 Hz, 2H), 2.58-2.53 (m, 4H), 2.40-1.87 (m, 9H), 1.69 (d, J=8.7 Hz, 1H), 1.48 (d, J=4.6 Hz, 1H), 0.83 (d, J=6.4 Hz, 3H). [M+H]⁺=826.7

### Example 344: (R,E)-N—((3S,4R)-1-(4-((R)-2,6-dioxopiperidin-3-yl)-3,5-difluorophenethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

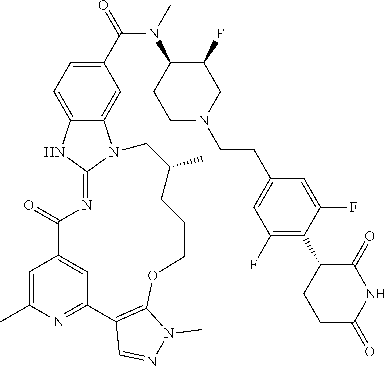 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@@H]3F)cc21]

The titled compound was prepared in a manner similar to that in Example 260.

¹H NMR (500 MHz, DMSO) δ 12.87 (s, 1¹H), 10.93 (s, 1H), 8.43 (s, 1¹H), 7.92 (s, 1¹H), 7.57 (d, J=5.3 Hz, 3H), 7.19 (s, 1H), 7.04 (s, 1H), 6.98 (s, 1H), 4.93-4.62 (m, 1H), 4.41-4.33 (m, 1H), 4.19 (d, J=12.2 Hz, 2H), 4.00 (d, J=3.8 Hz, 2H), 3.73 (s, 3H), 3.54-3.36 (m, 2H), 2.97-2.63 (m, 9H), 2.56 (s, 3H), 2.54-2.51 (m, 2H), 2.24-1.68 (m, 9H), 1.45 (s, 1H), 0.81 (d, J=6.3 Hz, 3H). [M+H]⁺=840.7

### Example 345: (R,E)-N—((3R,4R)-1-(4-((R)-2,6-dioxopiperidin-3-yl)-3,5-difluorophenethyl)-3-fluoropiperidin-4-yl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

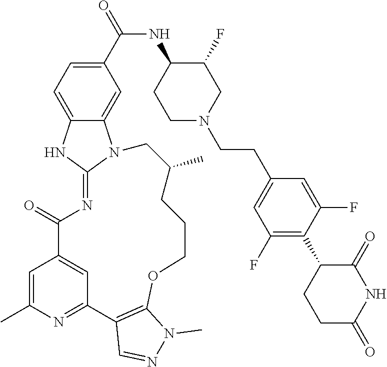 [SMILES: Cc1cc2cc(n1)-c1cnn(C)c1OCCC[C@@H](C)CN1/C(=N/C2=O)Nc2ccc(C(=O)N[C@@H]3CCN(CCc4cc(F)c([C@H]5CCC(=O)NC5=O)c(F)c4)C[C@H]3F)cc21]

The titled compound was prepared in a manner similar to that in Example 260.

¹H NMR (500 MHz, DMSO) δ 12.91 (s, 1H), 10.95 (s, 1H), 8.44 (s, 2H), 8.05 (s, 1H), 7.93 (s, 1H), 7.79 (d, J=8.5 Hz, 1H), 7.57 (d, J=8.1 Hz, 2H), 7.05 (d, J=10.1 Hz, 2H), 4.69-4.52 (m, 1H), 4.37 (d, J=4.7 Hz, 1H), 4.21 (dd, J=17.7, 9.5 Hz, 2H), 4.09-3.91 (m, 3H), 3.74 (s, 3H), 2.92 (d, J=11.1 Hz, 1H), 2.89-2.76 (m, 4H), 2.71-2.63 (m, 2H), 2.56 (s, 3H), 2.55 (d, J=3.8 Hz, 1H), 2.29-1.83 (m, 9H), 1.65-1.55 (m, 1H), 1.49 (d, J=7.8 Hz, 1H), 0.84 (d, J=6.4 Hz, 3H). [M+H]⁺=826.7

### Example 346: (R,E)-N—((3R,4R)-1-(4-((R)-2,6-dioxopiperidin-3-yl)-3,5-difluorophenethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

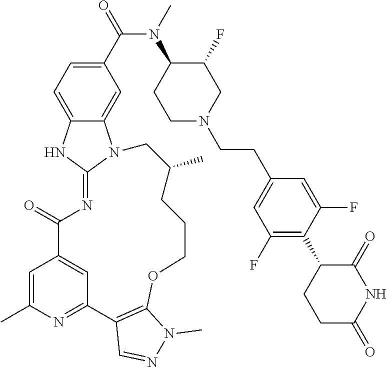 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3F)cc21]

### Step 1: tert-butyl (3R,4R)-4-(((benzyloxy)carbonyl)amino)-3-fluoropiperidine-1-carboxylate

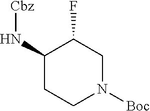 [SMILES: CC(C)(C)OC(=O)N1CC[C@@H](NC(=O)OCc2ccccc2)[C@H](F)C1]

To a solution of tert-butyl (3R,4R)-4-amino-3-fluoropiperidine-1-carboxylate (480 mg, 2.24 mmol) in DCM (10 mL) and DIEA (580 mg, 4.49 mmol) was added CbzCl (500 mg, 2.92 mmol) at 5-10° C. The reaction mixture was stirred at r.t for 3 hrs. The reaction was quenched with water (40 mL), the resulting mixture was extracted with DCM (10 mL×3). The combined organic phase was washed with brine (20 mL), dried over Na₂SO₄, filtered and concentrated in vacuum. The residue was purified by silica gel column (PE:EA=2:1) to afford tert-butyl (3R,4R)-4-(((benzyloxy)carbonyl)amino)-3-fluoropiperidine-1-carboxylate (720 mg, 91.1%). [M+H]⁺=353.2.

### Step 2: tert-butyl (3R,4R)-4-(((benzyloxy)carbonyl)(methyl)amino)-3-fluoropiperidine-1-carboxylate

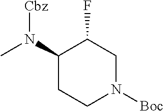 [SMILES: CN(C(=O)OCc1ccccc1)[C@@H]1CCN(C(=O)OC(C)(C)C)C[C@H]1F]

To a solution of tert-butyl (3R,4R)-4-(((benzyloxy)carbonyl)amino)-3-fluoropiperidine-1-carboxylate (720 mg, 2.05 mmol) in THF (10 mL) was added NaH (125 mg, 3.1 mmol, 60%). The reaction was stirred at r.t for 1h, then CH₃I (450 mg, 3.1 mmol) was added, and the resulting mixture was stirred at r.t for overnight. The reaction was quenched with sat. NH₄Cl solution (20 mL) at 0-10° C., and the resulting mixture was extracted with EA (20 mL×3). The combined organic phase was washed with brine (20 mL), dried over Na₂SO₄, filtered and concentrated in vacuum. The residue was purified by silica gel column (PE:EA=3:1) to afford tert-butyl (3R,4R)-4-(((benzyloxy)carbonyl)(methyl)amino)-3-fluoropiperidine-1-carboxylate (720 mg, 96.2%). [M+H]⁺=367.2.

### Step 3: tert-butyl (3R,4R)-3-fluoro-4-(methylamino)piperidine-1-carboxylate

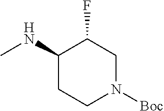 [SMILES: CN[C@@H]1CCN(C(=O)OC(C)(C)C)C[C@H]1F]

To a solution of tert-butyl (3R,4R)-4-(((benzyloxy)carbonyl)(methyl)amino)-3-fluoropiperidine-1-carboxylate (720 mg, 1.97 mmol) in THF (10 mL) was added Pd/C (250 mg, 10%). The mixture was stirred at r.t for 8 hrs under hydrogen atmosphere. The reaction mixture was filtered through celite, and the filtrate was concentrated in vacuo to afford tert-butyl (3R,4R)-3-fluoro-4-(methylamino)piperidine-1-carboxylate (430 mg, 93.7%). [M+H]⁺=233.2.

### Step 4: tert-butyl (3R,4R)-3-fluoro-4-((R,E)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate

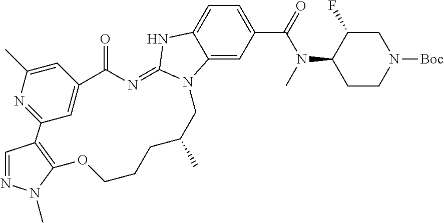 [SMILES: Cc1cc2cc(n1)-c1cnn(C)c1OCCC[C@@H](C)CN1/C(=N/C2=O)Nc2ccc(C(=O)N(C)[C@@H]3CCN(C(=O)OC(C)(C)C)C[C@H]3F)cc21]

To a mixture of (R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxylic acid (200 mg, 0.42 mmol) and tert-butyl (3R,4R)-3-fluoro-4-(methylamino)piperidine-1-carboxylate (132 mg, 0.55 mmol) in CH₃CN (10 mL) was added TCFH (177 mg, 0.63 mmol) and 1-methylimidazole (173 mg, 2.11 mmol). The reaction was stirred at r.t for 2 hrs. The reaction was quenched with H₂O (20 mL), the resulting mixture was extracted with DCM (10 mL×3). The combined organic phase was washed with brine (30 mL), dried over Na₂SO₄, filtered and concentrated in vacuum. The residue was purified by silica gel column (DCM:CH₃OH=20:1) to give the title compound (262 mg, 90.6%). [M+H]⁺=689.5.

### Step 5: (R,E)-N—((3R,4R)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

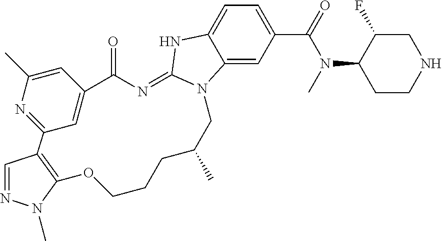 [SMILES: Cc1cc2cc(n1)-c1cnn(C)c1OCCC[C@@H](C)CN1/C(=N/C2=O)Nc2ccc(C(=O)N(C)[C@@H]3CCNC[C@H]3F)cc21]

To a solution of tert-butyl (3R,4R)-3-fluoro-4-((R,E)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamido)piperidine-1-carboxylate (262 mg, 0.38 mmol) in DCM (10 mL) was added TFA (1 mL). The reaction was stirred at r.t for 2 hrs. The reaction was concentrated in vacuo, the residue was dissolved in DCM (20 mL) and then washed with sat. NaHCO₃ solution (10 mL) and brine (10 mL), dried over Na₂SO₄, filtered and concentrated in vacuum to afford the title compound (205 mg, 90.5%). [M+H]⁺=589.3.

### Step 6: (R,E)-N—((3R,4R)-1-(4-((R)-2,6-dioxopiperidin-3-yl)-3,5-difluorophenethyl)-3-fluoropiperidin-4-yl)-N,1¹,2⁶,7-tetramethyl-3-oxo-5²,5³-dihydro-11H,5¹H-11-oxa-4-aza-5(2,1)-benzo[d]imidazola-2(2,4)-pyridina-1(4,5)-pyrazolacycloundecaphane-5⁶-carboxamide

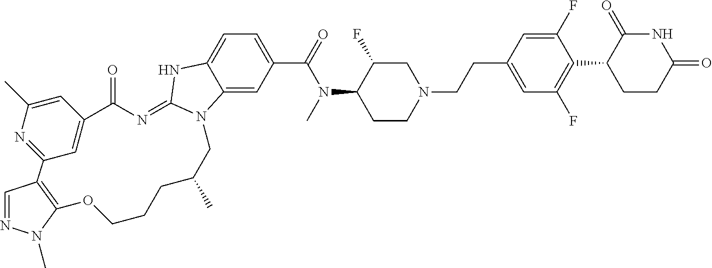 [SMILES: Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3F)cc21]

To a mixture of (R,E)-N—((3R,4R)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide (205 mg, 0.35 mmol) and (R)-2-(4-(2,6-dioxopiperidin-3-yl)-3,5-difluorophenyl)acetaldehyde (120 mg, 0.45 mmol) in DCE (10 mL) was added STAB (183 mg, 0.87 mmol). The reaction was stirred at rt for 2 hrs. The reaction was quenched with H₂O (20 mL), the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=20:1) to afford the title compound (125 mg, 43.0%). [M+H]⁺=840.4.

¹H NMR (500 MHz, DMSO) δ 12.87 (s, 1H), 10.93 (s, 1H), 8.43 (s, 1H), 7.92 (s, 1H), 7.57 (d, J=5.3 Hz, 3H), 7.19 (s, 1H), 7.04 (s, 1H), 6.98 (s, 1H), 4.93-4.62 (m, 1H), 4.35 (s, 1H), 4.19 (d, J=12.2 Hz, 2H), 4.00 (d, J=3.8 Hz, 2H), 3.73 (s, 3H), 3.54-3.36 (m, 2H), 2.97-2.63 (m, 9H), 2.56 (s, 3H), 2.53 (s, 2H), 2.24-1.68 (m, 9H), 1.45 (s, 1H), 0.81 (d, J=6.3 Hz, 3H). [M+H]⁺=840.7

### Example 350: (R,E)-N—((3S,4R)-1-(2-((4-((R)-2,6-dioxopiperidin-3-yl)-3,5-difluorophenyl)amino)ethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

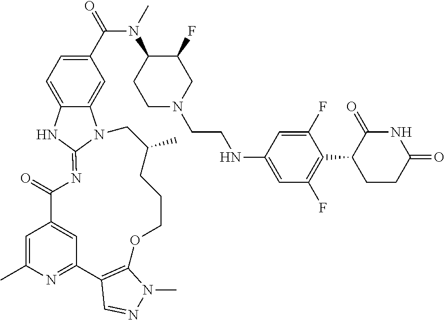 [SMILES: Cc1cc2cc(n1)-c1cnn(C)c1OCCC[C@@H](C)CN1/C(=N/C2=O)Nc2ccc(C(=O)N(C)[C@@H]3CCN(CCNc4cc(F)c([C@H]5CCC(=O)NC5=O)c(F)c4)C[C@@H]3F)cc21]

The titled compound was prepared in a manner similar to that in Example 260.

¹H NMR (500 MHz, DMSO) δ 12.87 (s, 1H), 10.82 (s, 1H), 8.43 (s, 1H), 7.92 (s, 1H), 7.73 (s, 1H), 7.57 (d, J=4.2 Hz, 2H), 7.27 (s, 1H), 6.25 (s, 2H), 6.06 (s, 1H), 4.96 (m, 1H), 4.41-4.31 (m, 2H), 4.19 (d, J=11.8 Hz, 1H), 4.08-3.93 (m, 3H), 3.73 (s, 3H), 3.48-3.41 (m, 2H), 3.19-3.05 (m, 3H), 3.02-2.86 (m, 4H), 2.85-2.72 (m, 2H), 2.66-2.54 (m, 6H), 2.29-2.18 (m, 2H), 2.12-1.87 (m, 5H), 1.45 (d, J=4.6 Hz, 1H), 0.82 (d, J=6.0 Hz, 3H); [M+H]⁺=855.7.

### Example 351: (R,E)-N—((3R,4S)-1-(2-((4-((R)-2,6-dioxopiperidin-3-yl)-3,5-difluorophenyl)amino)ethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

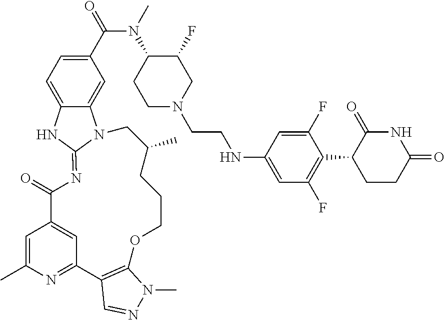 [SMILES: Cc1cc2cc(n1)-c1cnn(C)c1OCCC[C@@H](C)CN1/C(=N/C2=O)Nc2ccc(C(=O)N(C)[C@H]3CCN(CCNc4cc(F)c([C@H]5CCC(=O)NC5=O)c(F)c4)C[C@H]3F)cc21]

The titled compound was prepared in a manner similar to that in Example 260.

¹H NMR (500 MHz, DMSO) δ 12.90 (s, 1H), 10.84 (s, 1H), 8.44 (s, 1H), 7.94 (s, 1H), 7.73 (s, 1H), 7.59 (d, J=7.7 Hz, 2H), 7.31 (s, 1H), 6.44 (s, 1H), 6.33 (s, 2H), 5.38-5.29 (m, 1H), 4.36 (s, 1H), 4.20 (d, J=12.3 Hz, 1H), 4.02 (s, 3H), 3.74 (s, 3H), 3.50-3.45 (m, 4H), 2.96 (s, 3H), 2.78 (s, 2H), 2.64-2.47 (m, 6H), 2.46-2.39 (m, 3H), 2.22 (s, 1H), 2.08-1.92 (m, 6H), 1.45 (s, 1H), 0.83 (d, J=6.2 Hz, 3H). [M+H]⁺=855.7.

### Example 352: (R,E)-N—((3S,4S)-1-(2-((4-((R)-2,6-dioxopiperidin-3-yl)-3,5-difluorophenyl)amino)ethyl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

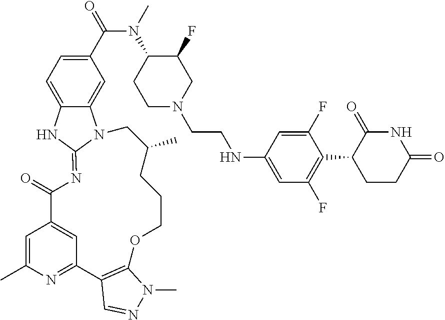 [SMILES: Cc1cc2cc(n1)-c1cnn(C)c1OCCC[C@@H](C)CN1/C(=N/C2=O)Nc2ccc(C(=O)N(C)[C@H]3CCN(CCNc4cc(F)c([C@H]5CCC(=O)NC5=O)c(F)c4)C[C@@H]3F)cc21]

The titled compound was prepared in a manner similar to that in Example 260.

¹H NMR (500 MHz, DMSO) δ 12.87 (s, 1H), 10.81 (s, 1H), 8.43 (s, 1H), 7.93 (s, 1H), 7.64 (s, 1H), 7.57 (d, J=4.1 Hz, 2H), 7.19 (s, 1H), 6.34-6.15 (m, 2H), 6.04 (s, 1H), 4.64-4.54 (m, 1H), 4.42-4.30 (m, 1H), 4.19 (d, J=12.9 Hz, 1H), 4.10-3.92 (m, 3H), 3.73 (s, 3H), 3.52-3.42 (m, 1H), 3.18 (s, 1H), 3.05 (s, 1H), 2.99-2.87 (m, 3H), 2.86-2.70 (m, 3H), 2.65-2.52 (m, 6H), 2.47-2.44 (m, 2H), 2.21 (s, 1H), 2.07-1.73 (m, 7H), 1.45 (s, 1H), 0.83 (d, J=6.2 Hz, 3H). [M+H]⁺=855.7.

### Example 354: (R)-3-(2,6-difluoro-4-(4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)azetidine-3-carbonyl) piperazin-1-yl)phenyl)piperidine-2,6-dione

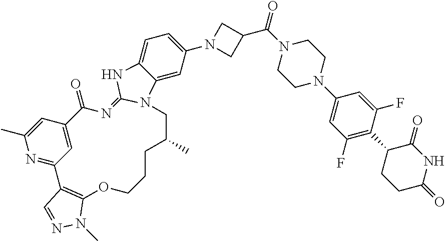 [SMILES: Cc1cc2cc(n1)-c1cnn(C)c1OCCC[C@@H](C)CN1/C(=N/C2=O)Nc2ccc(N3CC(C(=O)N4CCN(c5cc(F)c([C@H]6CCC(=O)NC6=O)c(F)c5)CC4)C3)cc21]

The titled compound was prepared in a manner similar to that in Example 333.

¹H NMR (500 MHz, DMSO) δ 12.59 (s, 1H), 10.89 (s, 1H), 8.58 (s, 1H), 8.13 (s, 1H), 7.78 (s, 1H), 7.38 (d, J=8.5 Hz, 1H), 6.70 (d, J=12.5 Hz, 3H), 6.41 (d, J=8.5 Hz, 1H), 4.45-4.35 (m, 1H), 4.18-4.02 (m, 5H), 3.94 (dd, J=13.7, 9.1 Hz, 4H), 3.76 (s, 3H), 3.60 (s, 3H), 3.28-3.23 (m, 6H), 2.82-2.77 (m, 2H), 2.68-2.64 (m, 3H), 2.25-2.17 (m, 1H), 2.17-2.04 (m, 1H), 2.02-1.88 (m, 3H), 1.50-1.45 (m, 1H), 0.82 (d, J=6.3 Hz, 3H); [M+H]⁺=821.8.

### Example 357: (R)-3-(2,6-difluoro-4-((S)-3-(methoxymethyl)-4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperazin-1-yl)phenyl)piperidine-2,6-dione

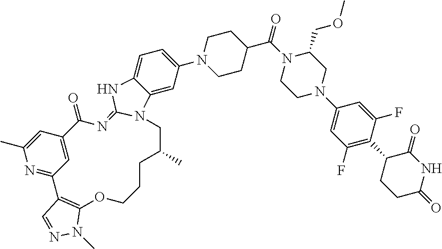 [SMILES: COC[C@@H]1CN(c2cc(F)c([C@H]3CCC(=O)NC3=O)c(F)c2)CCN1C(=O)C1CCN(c2ccc3c(c2)N2C[C@H](C)CCCOc4c(cnn4C)-c4cc(cc(C)n4)C(=O)/N=C/2N3)CC1]

The titled compound was prepared in a manner similar to that in Example 333.

¹H NMR (500 MHz, DMSO) δ 12.50 (s, 1H), 10.88 (s, 1H), 8.42 (s, 1H), 7.92 (s, 1H), 7.56 (s, 1H), 7.37 (d, J=8.7 Hz, 1H), 7.15 (s, 1H), 6.91 (d, J=8.8 Hz, 1H), 6.70-6.55 (m, 2H), 4.67 (m, 1H), 4.41-4.35 (m, 1H), 4.34-4.27 (m, 1H), 4.14 (d, J=12.8 Hz, 1H), 4.07 (dd, J=12.6, 4.3 Hz, 1H), 3.98 (m, 3H), 3.76 (s, 2H), 3.71 (s, 2H), 3.63-3.54 (m, 1H), 3.51-3.43 (m, 1H), 3.29 (s, 3H), 3.25 (s, 3H), 2.93 (dd, J=15.8, 9.3 Hz, 1H), 2.89-2.73 (m, 6H), 2.71-2.62 (m, 1H), 2.55 (s, 3H), 2.26 (s, 1H), 2.10 (s, 1H), 2.03-1.88 (m, 3H), 1.83-1.62 (m, 4H), 1.50 (s, 1H), 0.82 (d, J=6.3 Hz, 3H). [M+H]⁺=893.4

### Example 358: (R)-3-(2,6-difluoro-4-(4-((S)-1-((R,E)-1¹,2⁴,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)pyrrolidine-3-carbonyl)piperazin-1-yl)phenyl)piperidine-2,6-dione

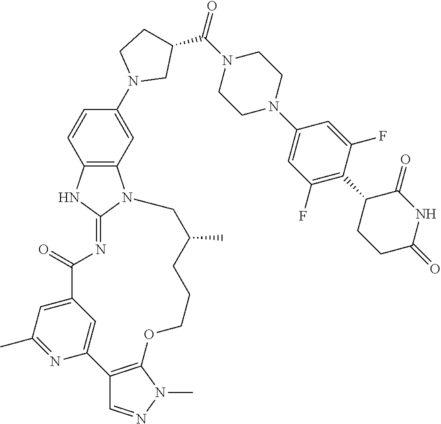 [SMILES: Cc1cc2cc(n1)-c1cnn(C)c1OCCC[C@@H](C)CN1/C(=N/C2=O)Nc2ccc(N3CC[C@H](C(=O)N4CCN(c5cc(F)c([C@H]6CCC(=O)NC6=O)c(F)c5)CC4)C3)cc21]

The titled compound was prepared in a manner similar to that in Example 333.

¹H NMR (500 MHz, DMSO) δ 12.43 (s, 1H), 10.89 (s, 1H), 8.42 (s, 1H), 7.92 (s, 1H), 7.55 (s, 1H), 7.35 (d, J=8.6 Hz, 1H), 6.71 (d, J=12.2 Hz, 3H), 6.52 (d, J=4.9 Hz, 1H), 4.36-4.33 (m, 1H), 4.15-4.12 (d, 1H), 4.08-4.05 (m, 1H), 3.96-3.93 (m, 2H), 3.73-3.69 (m, 5H), 3.67-3.59 (m, 3H), 3.51-3.48 (m, 2H), 3.47-3.41 (m, 2H), 3.21-3.19 (m, 2H), 2.87-2.74 (m, 2H), 2.55-2.53 (m, 3H), 2.28-2.05 (m, 4H), 1.97-1.95 (m, 3H), 1.52-1.40 (m, 1H), 1.24-1.21 (m, 2H), 0.88-0.79 (m, 4H); [M+H]⁺=835.7.

### Example 359: (R,E)-N—((S)-1-(1-(4-((R)-2,6-dioxopiperidin-3-yl)-3,5-difluorophenyl)azetidin-3-yl)-3,3-difluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

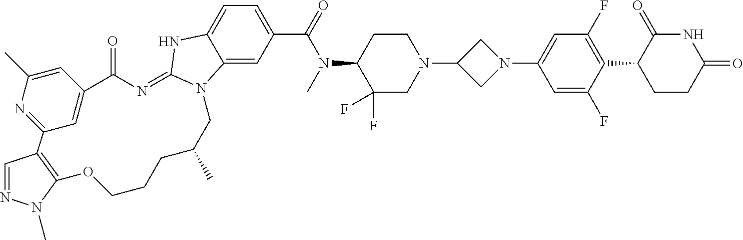 [SMILES: Cc1cc2cc(n1)-c1cnn(C)c1OCCC[C@@H](C)CN1/C(=N/C2=O)Nc2ccc(C(=O)N(C)[C@H]3CCN(C4CN(c5cc(F)c([C@H]6CCC(=O)NC6=O)c(F)c5)C4)CC3(F)F)cc21]

The titled compound was prepared in a manner similar to that in Example 260.

¹H NMR (500 MHz, DMSO) δ 12.88 (s, 1H), 10.85 (s, 1H), 8.43 (s, 1H), 7.93 (s, 1H), 7.69 (s, 1H), 7.58 (d, J=7.8 Hz, 2H), 7.24 (s, 1H), 6.09 (s, 2H), 4.38-4.34 (m, 1H), 4.23-4.18 (m, 1H), 4.11-3.96 (m, 4H), 3.95-3.75 (m, 3H), 3.73 (s, 3H), 3.70-3.54 (m, 2H), 3.06-2.80 (m, 5H), 2.77-2.60 (m, 2H), 2.56 (s, 3H), 2.48-2.31 (m, 2H), 2.20-2.15 (m, 2H), 2.11-1.73 (m, 6H), 1.47-1.43 (m, 1H), 0.84-0.83 (m, 3H); [M+H]⁺=885.6.

### Example 363: (R)-3-(2,6-difluoro-4-((R)-2-(methoxymethyl)-4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperazin-1-yl)phenyl)piperidine-2,6-Dione

### Step 1: tert-butyl (R)-4-(4-(2,6-bis(benzyloxy)pyridin-3-yl)-3,5-difluorophenyl-3-(methoxymethyl)piperazine-1-carboxylate

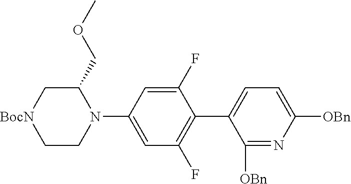 [SMILES: COC[C@H]1CN(C(=O)OC(C)(C)C)CCN1c1cc(F)c(-c2ccc(OCc3ccccc3)nc2OCc2ccccc2)c(F)c1]

To a solution of 2,6-bis(benzyloxy)-3-(4-bromo-2,6-difluorophenyl)pyridine (3 g, 6.22 mmol) in dioxane (30 mL) were added tert-butyl (R)-3-(methoxymethyl)piperazine-1-carboxylate (1.71 g, 7.47 mmol), Pd₂(dba)₃ (567.3 mg, 0.62 mmol), Ruphos (581 mg, 1.25 mmol), Cs₂CO₃(6.1 g, 18.67 mmol), The resulting solution was stirred for 2 h at 100° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (10:1) to afford tert-butyl (R)-4-(4-(2,6-bis(benzyloxy)pyridin-3-yl)-3,5-difluorophenyl)-3-(methoxymethyl)piperazine-1-carboxylate (2.5 g, 64.1%). [M+H]⁺=632.3.

### Step 2: tert-butyl (3R)-4-(4-(2,6-dioxopiperidin-3-yl)-3,5-difluorophenyl)-3-(methoxymethyl)piperazine-1-carboxylate

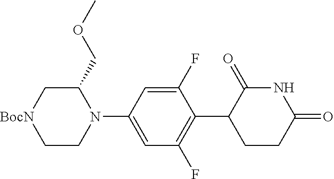 [SMILES: COC[C@H]1CN(C(=O)OC(C)(C)C)CCN1c1cc(F)c(C2CCC(=O)NC2=O)c(F)c1]

A round bottom flask equipped with a magnetic stirrer, were charged with tert-butyl (R)-4-(4-(2,6-bis(benzyloxy)pyridin-3-yl)-3,5-difluorophenyl)-3-(methoxymethyl)piperazine-1-carboxylate (2.5 g, 3.96 mmol), dry THF (30 m1), and Pd/C (10 wt %, 2.5 g). The resulting mixture was degassed under reduced pressure and purged with H₂ for five times, then stirred overnight at 40° C. The mixture was diluted with THF, then sonicated in an ultrasonic washer for 10 minutes, followed by filtration through a pad of celite. The filtrate was concentrated under vacuum, The residue was purified by silica column chromatography (EA:PE=0-40%) to afford tert-butyl (3R)-4-(4-(2,6-dioxopiperidin-3-yl)-3,5-difluorophenyl)-3-(methoxymethyl)piperazine-1-carboxylate (1.5 g, 83.8%). [M+H]⁺=454.2.

### Step 3: tert-butyl (R)-4-(4-((R)-2,6-dioxopiperidin-3-yl)-3,5-difluorophenyl)-3-(methoxymethyl)piperazine-1-carboxylate

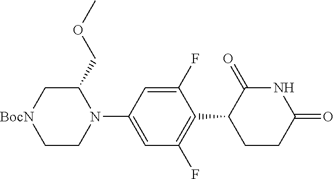 [SMILES: COC[C@H]1CN(C(=O)OC(C)(C)C)CCN1c1cc(F)c([C@H]2CCC(=O)NC2=O)c(F)c1]

The tert-butyl (3R)-4-(4-(2,6-dioxopiperidin-3-yl)-3,5-difluorophenyl)-3-(methoxymethyl)piperazine-1-carboxylate (1.8 g) was separated by Prer-chiral-SFC with following condition: (Column: Chiral ND (2) 3.0*100 mm, 3 μm; Flow rate: 2 mL/min; Gradient: 10% to 50% in 2.0 min, hold 1.0 min at 50%, Injection Volume:0.2 ul L) to afford tert-butyl (R)-4-(4-((R)-2,6-dioxopiperidin-3-yl)-3,5-difluorophenyl)-3-(methoxymethyl)piperazine-1-carboxylate (509.4 mg, 56.6%, ee=98.88%). [M+H]⁺=454.2.

1H NMR (300 MHz, Chloroform-d) δ 8.20 (s, 1H), 6.41 (d, J=15 Hz, 2H), 4.11-3.91 (m, 3H), 3.77 (s, 1H), 3.36 (d, J=9 Hz, 6H), 3.12 (t, J=18 Hz, 3H), 2.72 (m, 2H), 2.41-2.01 (m, 2H), 1.49 (s, 9H).

### Step 4: tert-butyl (R)-4-(4-((S)-2,6-dioxopiperidin-3-yl)-3,5-difluorophenyl)-3-(methoxymethyl)piperazine-1-carboxylate

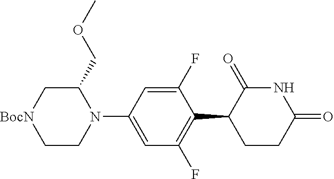 [SMILES: COC[C@H]1CN(C(=O)OC(C)(C)C)CCN1c1cc(F)c([C@@H]2CCC(=O)NC2=O)c(F)c1]

The tert-butyl (3R)-4-(4-(2,6-dioxopiperidin-3-yl)-3,5-difluorophenyl)-3-(methoxymethyl)piperazine-1-carboxylate (1.8 g) was separated by Prer-chiral-SFC with following condition: (Column: Chiral ND (2) 3.0*100 m m, 3 μm; Flow rate: 2 mL/min; Gradient: 10% to 50% in 2.0 min, hold 1.0 min at 50%, Injection Volume:0.2 ul L) to afford tert-butyl (R)-4-(4-((S)-2,6-dioxopiperidin-3-yl)-3,5-difluorophenyl)-3-(methoxymethyl)piperazine-1-carboxylate (404.8 mg, 44.9% ee=97.06%). [M+H]⁺=454.2. ¹H NMR (300 MHz. Chloroform-d) S 8.20 (s, 1H), 6.41 (d, J=15 Hz, 2H), 4.11-3.91 (m, 3H), 3.77 (s, 1H), 3.36 (d, J=9 Hz, 6H), 3.12 (t, J=18 Hz, 3H), 2.72 (m, 2H), 2.41-2.01 (m, 2H), 1.49 (s, 9H).

### Step 5: (R)-3-(2,6-difluoro-4-((R)-2-(methoxymethyl)piperazin-1-yl)phenyl)piperidine-2,6-dione

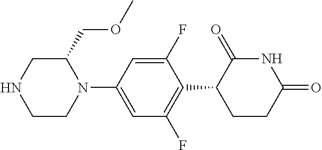 [SMILES: COC[C@H]1CNCCN1c1cc(F)c([C@H]2CCC(=O)NC2=O)c(F)c1]

A solution of tert-butyl (R)-4-(4-((R)-2,6-dioxopiperidin-3-yl)-3,5-difluorophenyl)-3-(methoxymethyl)piperazine-1-carboxylate (500 mg, 1.10 mmol) in DCM (10 mL) was added HCl (4M in dioxane) (3 mL). The mixture was stirred in a flask at room temperature for 2 h. The mixture was evaporated in vacuum to afford the crude product (360 mg, 92.4%), which was used for next step without further purification.

### Step 6: (R)-3-(2,6-difluoro-4-((R)-2-(methoxymethyl)-4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperazin-1-yl)phenyl)piperidine-2,6-Dione

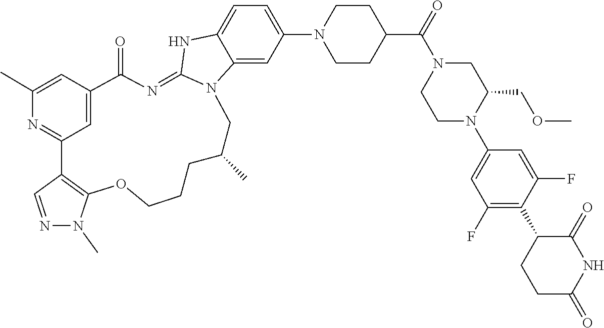 [SMILES: COC[C@H]1CN(C(=O)C2CCN(c3ccc4c(c3)N3C[C@H](C)CCCOc5c(cnn5C)-c5cc(cc(C)n5)C(=O)/N=C/3N4)CC2)CCN1c1cc(F)c([C@H]2CCC(=O)NC2=O)c(F)c1]

The titled compound was prepared in a manner similar to that in Example 333.

¹H NMR (500 MHz, DMSO-d₆) δ 12.50 (s, 1H), 10.88 (s, 1H), 8.43 (s, 1H), 7.92 (s, 1H), 7.56 (s, 1H), 7.37 (d, J=8.7 Hz, 1H), 7.17 (s, 1H), 6.92 (d, J=6.5 Hz, 1H), 6.62 (t, J=11.7 Hz, 2H), 4.52-4.41 (m, 1H), 4.42-4.32 (m, 1H), 4.30-4.22 (m, 1H), 4.19-4.09 (m, 2H), 4.09-4.02 (m, 3H), 4.03-3.93 (m, 3H), 3.82-3.74 (m, 2H), 3.73 (s, 3H), 3.20 (s, 3H), 3.05-2.89 (m, 3H), 2.86-2.70 (m, 6H), 2.55 (s, 3H), 2.22 (d, J=9.2 Hz, 1H), 2.16-2.05 (m, 1H), 2.03-1.89 (m, 3H), 1.75 (s, 4H), 1.47 (s, 1H), 0.82 (d, J=6.3 Hz, 3H). [M+H]⁺=893.4

### Example 364: (R)-3-(2,6-difluoro-4-((1-(1-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperidin-4-yl)oxy)phenyl)piperidine-2,6-dione

### Step 1: 2,6-bis(benzyloxy)-3-(2,6-difluoro-4-(4,4,5,5-tetramethyl-1,3,2-dioxaborolan-2-yl)phenyl)pyridine

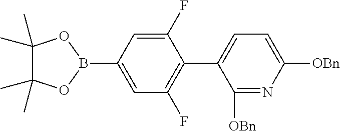 [SMILES: CC1(C)OB(c2cc(F)c(-c3ccc(OCc4ccccc4)nc3OCc3ccccc3)c(F)c2)OC1(C)C]

To a solution of 2,6-bis(benzyloxy)-3-(4-bromo-2,6-difluorophenyl)pyridine (35 g, 72.76 mmol) in dioxane (300 mL) were added B(pin)₂ (36.95 g, 145.50 mmol), Pd(dppf)Cl₂ (5.28 g, 7.30 mmol), K₂CO₃ (30.12 g, 218.29 mmol). The resulting solution was stirred overnight at 100° C. under N₂ atmosphere. After cooled to room temperature and filtered, the filter cake was washed with EA. The filtrate was concentrated under reduced pressure. The crude product purified by silica column chromatography (EA:PE=0-10%) to afford 2,6-bis(benzyloxy)-3-(2,6-difluoro-4-(4,4,5,5-tetramethyl-1,3,2-dioxaborolan-2-yl)phenyl)pyridine (30 g, 77.94%). [M+H]⁺=530.39

### Step 2: 4-(2,6-bis(benzyloxy)pyridin-3-yl)-3,5-difluorophenol

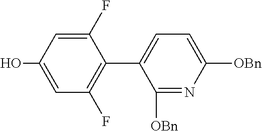 [SMILES: Oc1cc(F)c(-c2ccc(OCc3ccccc3)nc2OCc2ccccc2)c(F)c1]

To a stirred mixture of 2,6-bis(benzyloxy)-3-(2,6-difluoro-4-(4,4,5,5-tetramethyl-1,3,2-dioxaborolan-2-yl)phenyl)pyridine (30 g, 56.71 mmol) in AcOH (100 mL) and THF (100 mL) were added H₂O₂ (100 mL) in portions at 0° C. The mixture was stirred overnight at rt. Then saturated Na₂S₂O₃ aqueous solution was added and then the mixture was extracted with EA. The organic layer was washed with brine, dried over anhydrous Na₂SO₄. After filtration, the filtrate was concentrated under reduced pressure and purified by silica column chromatography (EA:PE=0-30%) to afford 4-(2,6-bis(benzyloxy)pyridin-3-yl)-3,5-difluorophenol (21 g, 88.38%). [M+H]⁺=420.43.

### Step 3: tert-butyl 4-(4-(2,6-bis(benzyloxy)pyridin-3-yl)-3,5-difluorophenoxy)piperidine-1-carboxylate

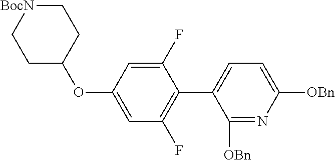 [SMILES: CC(C)(C)OC(=O)N1CCC(Oc2cc(F)c(-c3ccc(OCc4ccccc4)nc3OCc3ccccc3)c(F)c2)CC1]

A 500 mL RBF equipped with a magnetic stirrer, were charged with 4-(2,6-bis(benzyloxy)pyridin-3-yl)-3,5-difluorophenol (20.5 g, 48.9 mmol), tert-butyl 4-((methylsulfonyl)oxy)piperidine-1-carboxylate (20.5 g, 73.4 mmol), Cs₂CO₃ (47.8 g, 146.6 mmol), and DMF (210 mL). The resulting mixture was degassed under reduced pressure and purged with N2 for three times, then stirred at 110° C. for 2 hours. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by column chromatography (PE/EA=10:1 to 5:1) to afford tert-butyl 4-(4-(2,6-bis(benzyloxy)pyridin-3-yl)-3,5-difluorophenoxy)piperidine-1-carboxylate (14 g, 23.3 mmol, 47.65%). [M+H]⁺=603.1.

### Step 4: tert-butyl 4-(4-(2,6-dioxopiperidin-3-yl)-3,5-difluorophenoxy)piperidine-1-carboxylate

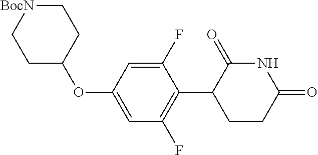 [SMILES: CC(C)(C)OC(=O)N1CCC(Oc2cc(F)c(C3CCC(=O)NC3=O)c(F)c2)CC1]

A 500 mL RBF equipped with a magnetic stirrer, were charged with tert-butyl 4-(4-(2,6-bis(benzyloxy)pyridin-3-yl)-3,5-difluorophenoxy)piperidine-1-carboxylate (14 g, 23.3 mmol), dry THF (240 ml), and Pd/C (10 wt %, 27 g). The resulting mixture was degassed under reduced pressure and purged with H₂ for five times, then stirred overnight at 50° C. The mixture was diluted with THF/DCM/MEOH (200 mL/200 mL/200 mL), then sonicated in an ultrasonic washer for 5 minutes, followed by filtration through a pad of celite. The filtrate was concentrated under vacuum. The residue was purified by column chromatography (PE/EA, 40-50%) to afford tert-butyl 4-(4-(2,6-dioxopiperidin-3-yl)-3,5-difluorophenoxy)piperidine-1-carboxylate (6.9 g, 69.96%). [M+H]⁺=425.

### Step 5: tert-butyl (R)-4-(4-(2,6-dioxopiperidin-3-yl)-3,5-difluorophenoxy)piperidine-1-carboxylate

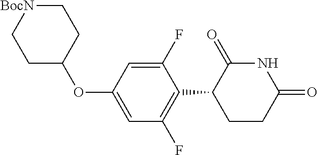 [SMILES: CC(C)(C)OC(=O)N1CCC(Oc2cc(F)c([C@H]3CCC(=O)NC3=O)c(F)c2)CC1]

The crude product (6.7 g) was purified by Prep-SFC with the following conditions (10% to 50% in 2.0 min, hold 1.0 min at 50%, MeOH (0.1% DEA)) to afford tert-butyl (R)-4-(4-(2,6-dioxopiperidin-3-yl)-3,5-difluorophenoxy)piperidine-1-carboxylate. [M+H]⁺=425.

### Step 6: tert-butyl (S)-4-(4-(2,6-dioxopiperidin-3-yl)-3,5-difluorophenoxy)piperidine-carboxylate

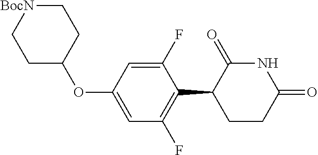 [SMILES: CC(C)(C)OC(=O)N1CCC(Oc2cc(F)c([C@@H]3CCC(=O)NC3=O)c(F)c2)CC1]

The crude product (obtained from step 5, 6.4 g) was purified by Prep-SFC with the following conditions (10% to 50% in 2.0 min, hold 1.0 min at 50%, MeOH (0.1% DEA)) to afford tert-butyl (S)-4-(4-(2,6-dioxopiperidin-3-yl)-3,5-difluorophenoxy)piperidine-1-carboxylate.[M+H]⁺=425

### Step 7: (R)-3-(2,6-difluoro-4-(piperidin-4-yloxy)phenyl)piperidine-2,6-dione

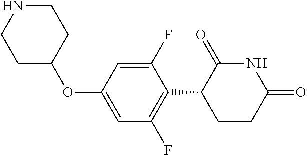 [SMILES: O=C1CC[C@H](c2c(F)cc(OC3CCNCC3)cc2F)C(=O)N1]

A solution of tert-butyl (R)-4-(4-(2,6-dioxopiperidin-3-yl)-3,5-difluorophenoxy)piperidine-1-carboxylate (500 mg, 1.18 mmol) in DCM (10 mL) was added HCl (4M in dioxane) (3 mL). The mixture was stirred in a flask at room temperature for 2 h. The mixture was evaporated in vacuum to afford the crude product (350 mg, 91.6%), which was used for next step without further purification. [M+H]⁺=325.

### Step 8: (R)-3-(2,6-difluoro-4-((1-(14(R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)piperidine-4-carbonyl)piperidin-4-yl)oxy)phenyl)piperidine-2,6-dione

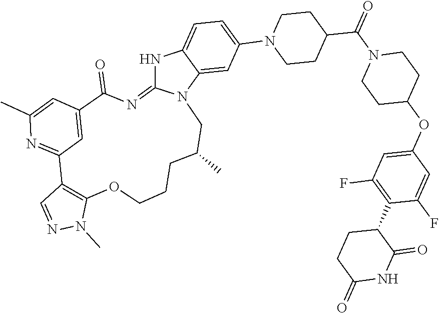 [SMILES: Cc1cc2cc(n1)-c1cnn(C)c1OCCC[C@@H](C)CN1/C(=N/C2=O)Nc2ccc(N3CCC(C(=O)N4CCC(Oc5cc(F)c([C@H]6CCC(=O)NC6=O)c(F)c5)CC4)CC3)cc21]

The titled compound was prepared in a manner similar to that in Example 333.

¹H NMR (500 MHz, DMSO-d₆) δ 12.50 (s, 1H), 10.93 (s, 1H), 8.42 (s, 1H), 7.92 (s, 1H), 7.56 (s, 1H), 7.37 (d, J=8.7 Hz, 1H), 7.15 (s, 1H), 6.90 (d, J=8.1 Hz, 1H), 6.84 (d, J=10.9 Hz, 2H), 4.75-4.65 (m, 1H), 4.42-4.31 (m, 1H), 4.14 (dd, J=12.9, 2.7 Hz, 2H), 4.04-3.96 (m, 2H), 3.94-3.88 (m, 1H), 3.84 (d, J=12.6 Hz, 2H), 3.75 (s, 1H), 3.73 (s, 3H), 3.69 (d, J=12.9 Hz, 1H), 2.89-2.70 (m, 6H), 2.55 (s, 3H), 2.26-2.17 (m, 1H), 2.17-2.06 (m, 2H), 2.05-1.96 (m, 3H), 1.93 (d, J=11.4 Hz, 2H), 1.70 (s, 4H), 1.57 (s, 1H), 1.44 (s, 2H), 0.82 (d, J=6.3 Hz, 3H). [M+H]⁺=864.4

### Example 365: 1-(4-((R)-2,6-dioxopiperidin-3-yl)-3,5-difluorophenethyl)-N-methyl-N—((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-(4,5)-pyrazolacycloundecaphane-5⁶-yl)piperidine-4-carboxamide

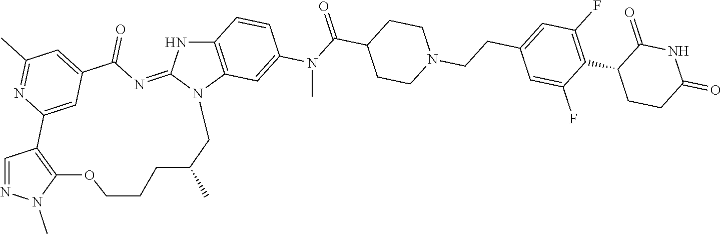 [SMILES: Cc1cc2cc(n1)-c1cnn(C)c1OCCC[C@@H](C)CN1/C(=N/C2=O)Nc2ccc(N(C)C(=O)C3CCN(CCc4cc(F)c([C@H]5CCC(=O)NC5=O)c(F)c4)CC3)cc21]

The titled compound was prepared in a manner similar to that in Example 260.

¹H NMR (500 MHz, DMSO) δ 12.85 (s, 1H), 10.92 (s, 1H), 8.43 (s, 1H), 7.92 (s, 1H), 7.74 (s, 1H), 7.58 (d, J=4.6 Hz, 2H), 7.19 (d, J=8.4 Hz, 1H), 6.94 (d, J=10.2 Hz, 2H), 4.36-4.33 (m, 1H), 4.26-4.13 (m, 2H), 3.99-3.96 (m, 2H), 3.73-3.68 (m, 3H), 3.19-3.16 (m, 3H), 2.79-2.76 (m, 4H), 2.64-2.61 (m, 2H), 2.56-2.51 (m, 3H), 2.36-2.32 (m, 3H), 2.20-2.18 (m, 1H), 2.09-2.06 (m, 2H), 2.02-1.86 (m, 3H), 1.67-1.38 (m, 7H), 0.80 (d, J=6.3 Hz, 3H); [M+H]⁺=822.6.

### Example 366: (R)-3-(2,6-difluoro-4-(2-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)phenyl)piperidine-2,6-dione

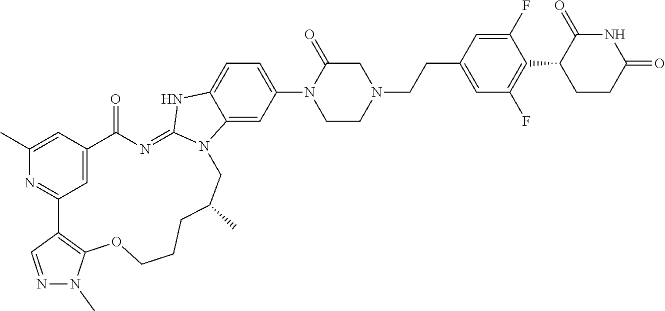 [SMILES: Cc1cc2cc(n1)-c1cnn(C)c1OCCC[C@@H](C)CN1/C(=N/C2=O)Nc2ccc(N3CCN(CCc4cc(F)c([C@H]5CCC(=O)NC5=O)c(F)c4)CC3=O)cc21]

The titled compound was prepared in a manner similar to that in Example 374.

¹H NMR (500 MHz, DMSO) δ 12.75 (s, 1H), 10.96 (s, 1H), 8.43 (s, 1H), 7.92 (s, 1H), 7.66 (s, 1H), 7.57 (s, 1H), 7.52 (d, J=8.4 Hz, 1H), 7.17 (d, J=8.4 Hz, 1H), 7.09 (d, J=10.1 Hz, 2H), 4.36 (d, J=4.5 Hz, 1H), 4.27-4.14 (m, 2H), 4.01 (d, 0.1=4.6 Hz, 1H), 3.98-3.91 (m, 1H), 3.73 (s, 3H), 3.73-3.70 (m, 1H), 3.69-3.62 (m, 1H), 3.28 (s, 2H), 2.89 (s, 2H), 2.84 (d, J=7.5 Hz, 4H), 2.73 (s, 2H), 2.56 (s, 3H), 2.52 (s, 1H), 2.26-2.09 (m, 2H), 2.05-1.87 (m, 3H), 1.43 (s, 1H), 0.82 (d, J=6.4 Hz, 3H). [M+H]⁺=780.7.

### Example 367: (R)-3-(4-(4-(1-((71R,72S,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperidine-4-carbonyl)piperazin-1-yl)-2,6-difluorophenyl)piperidine-2,6-dione

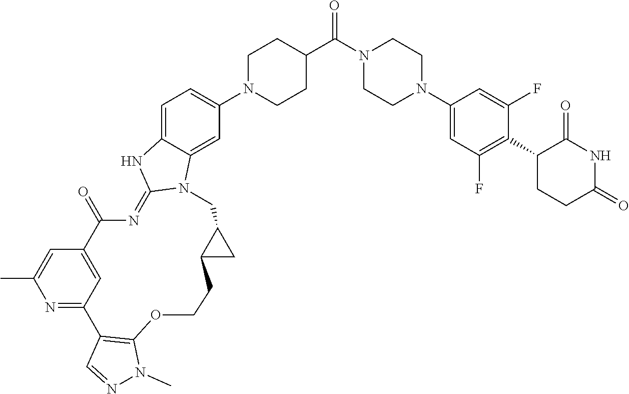 [SMILES: Cc1cc2cc(n1)-c1cnn(C)c1OCC[C@@H]1C[C@H]1CN1/C(=N/C2=O)Nc2ccc(N3CCC(C(=O)N4CCN(c5cc(F)c([C@H]6CCC(=O)NC6=O)c(F)c5)CC4)CC3)cc21]

The titled compound was prepared in a manner similar to that in Example 333.

¹H NMR (500 MHz, DMSO) δ 12.43 (s, 1H), 10.88 (s, 1H), 8.71 (s, 1H), 7.88 (s, 1H), 7.50 (s, 1H), 7.34 (d, J=8.6 Hz, 1H), 7.06 (s, 1H), 6.91 (d, J=8.6 Hz, 1H), 6.69 (d, J=12.6 Hz, 2H), 4.29 (q, J=12.7 Hz, 3H), 4.08 (dd, J=18.7, 6.4 Hz, 2H), 3.75 (s, 3H), 3.70 (d, J=17.9 Hz, 2H), 3.59 (s, 2H), 3.20 (s, 2H), 3.04-2.97 (m, 2H), 2.89-2.75 (m, 4H), 2.55 (s, 3H), 2.15-2.04 (m, 2H), 1.97 (d, J=5.3 Hz, 1H), 1.76 (s, 4H), 1.64 (d, J=5.6 Hz, 2H), 1.55 (d, J=5.2 Hz, 1H), 1.44 (s, 1H), 1.33 (s, 1H), 1.15 (d, J=4.0 Hz, 1H), 0.53 (s, 1H), 0.37 (d, J=4.4 Hz, 1H). [M+H]⁺=847.7

### Example 368: (R)-3-(2,6-difluoro-4-((2-(4-((R,E)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)amino)phenyl)piperidine-2,6-dione

### Step 1: 2-((4-(2,6-bis(benzyloxy)pyridin-3-yl)-3,5-difluorophenyl)amino)ethan-1-ol

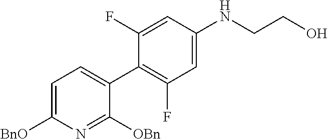 [SMILES: OCCNc1cc(F)c(-c2ccc(OCc3ccccc3)nc2OCc2ccccc2)c(F)c1]

To the solution of 2,6-bis(benzyloxy)-3-(4-bromo-2,6-difluorophenyl)pyridine (180 g, 374 mmol), 2-aminoethan-1-ol (45.6 g, 748 mmol) and K₃PO₄ (158.6 g, 748 mmol) in 1 L DMSO, CuI (7.1 g, 37.4 mmol) and L-Proline (4.3 g, 37.4 mmol) was added under N₂ protected. The mixture was stirred at 90° C. for 16 hours. The mixture was diluted with EtOAc and filtered. The filtrate was washed with saturated NaCl aqueous and concentrated in vacuum. The crude was purified with silica column chromatography (EA:PE=0-70%) to afford the title compound (109 g, 63% yield). [M+H]⁺=463.6.

### Step 2: 3-(2,6-difluoro-4-((2-hydroxyethyl)amino)phenyl)piperidine-2,6-dione

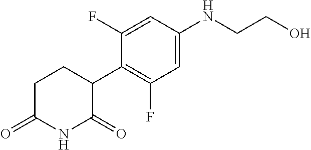 [SMILES: O=C1CCC(c2c(F)cc(NCCO)cc2F)C(=O)N1]

To the solution of 2-((4-(2,6-bis(benzyloxy)pyridin-3-yl)-3,5-difluorophenyl)amino)ethan-1-ol (108 g, 234 mmol) in 200 mL DCM and 1600 mL IPA, Pd/C (108 g, 10% w.t.) was added. The mixture was stirred at 45° C. for 16 hours at H₂ atmosphere. The mixture was filtered by celite. The filter cake was washed with DMF 2 times and the filtrate was combined and concentrated in vacuum to afford the title compound (45.6 g, 68% yield). [M+H]⁺=285.6.

### Step 3: 2-((4-(2,6-dioxopiperidin-3-yl)-3,5-difluorophenyl)amino)ethyl methanesulfonate

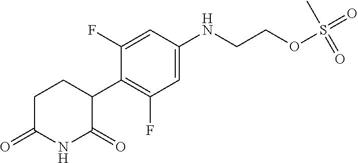 [SMILES: CS(=O)(=O)OCCNc1cc(F)c(C2CCC(=O)NC2=O)c(F)c1]

To the solution of 3-(2,6-difluoro-4-((2-hydroxyethyl)amino)phenyl)piperidine-2,6-dione (5.1 g, 18 mmol) and Et₃N (5.4 g, 54 mmol) in 100 mL DCM, MsCl (4.1 g, 36 mmol) was added. The mixture was stirred at room temperature for 1 hour. After LCMS shown the reaction was completed. The mixture was concentrated in vacuum and purified with silica column chromatography (MeOH:DCM=0-3%) to afford the title compound (4.2 g, 64% yield). [M+H]¹=363.6.

### Step 4: (R)-3-(2,6-difluoro-4-((2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-ylpiperazin-1-yl)ethyl)amino)phenyl)piperidine-2,6-dione

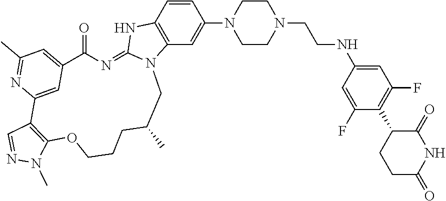 [SMILES: Cc1cc2cc(n1)-c1cnn(C)c1OCCC[C@@H](C)CN1/C(=N/C2=O)Nc2ccc(N3CCN(CCNc4cc(F)c([C@H]5CCC(=O)NC5=O)c(F)c4)CC3)cc21]

To the solution of (R,E)-1¹,2⁶,7-trimethyl-5⁶-(piperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (4 g, 7.77 mmol), 2-((4-(2,6-dioxopiperidin-3-yl)-3,5-difluorophenyl)amino)ethyl methanesulfonate (3.38 g, 9.33 mmol), KI (6.45 g, 38.86 mmol) and DIEA (8.04 g, 62.18 mmol) in 200 mL ACN and 15 mL DMSO. The mixture was stirred at 85° C. for 16 hours. After LCMS shown the reaction was completed. The mixture was concentrated in vacuum and purified with silica column chromatography (MeOH:DCM=0-5%) to afford the racemic (4.8 g, 79.2% yield). The racemic was separated with SFC (IF (2*25 cm, 5 um), 50% MtBE/50% (MeOH:DCM=1:1), 100 bar, 20 ml/min) and the title compound corresponded to peak A (@ 2.524 min/254 nm (1.931 g).

1H NMR (500 MHz, DMSO) δ 12.51 (s, 1H), 10.83 (s, 1H), 8.42 (s, 1H), 7.92 (s, 1H), 7.56 (s, 1H), 7.38 (d, J=8.7 Hz, 1H), 7.16 (s, 1H), 6.90 (d, J=8.6 Hz, 1H), 6.30 (d, J=12.0 Hz, 2H), 6.12 (s, 1H), 4.40-4.32 (m, 1H), 4.22-4.19 (m, 1H), 4.02-3.94 (m, 3H), 3.73 (s, 3H), 3.24-3.13 (m, 6H), 2.87-2.72 (m, 3H), 2.68-2.53 (m, 8H), 2.27-2.17 (m, 1H), 2.12-1.86 (m, 5H), 1.50-1.40 (m, 1H), 0.82 (d, J=6.4 Hz, 3H); [M+H]⁺=781.5.

### Example 373: 3-(5-(4-(4-((R,E)-1¹,2⁶,7-trimethyl-3-oxo-5²,5¹-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)pyridin-2-yl)piperidine-2,6-dione

### Step 1: 2′,6′-bis(benzyloxy)-5-bromo-2,3′-bipyridine

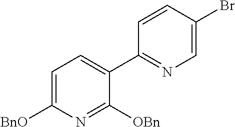 [SMILES: Brc1ccc(-c2ccc(OCc3ccccc3)nc2OCc2ccccc2)nc1]

To the solution of 5-bromo-2-iodopyridine (10 g, 35.2 mmol), 2,6-bis(benzyloxy)-3-(4,4,5,5-tetramethyl-1,3,2-dioxaborolan-2-yl)pyridine (14.7 g, 35.2 mmol) and K₂CO₃ (9.6 g, 70.4 mmol) in 1,4-dioxane (200 m1) and water (20 m1), were added Pd(PPh₃)₄ (3.52 mmol, 4.05 g). The mixture was stirred at 90° C. for 24 hours. The mixture was diluted with EA, and washed with water and brine. The organic layer was concentrated under reduced pressure to give the crude residue, which was purified by silica column chromatography (PE:EA=1:1) to afford the product (1¹ g, 70.1%). [M+H]⁺=447.1.

### Step 2: 8-(2′,6′-bis(benzyloxy)-[2,3′-bipyridin]-5-yl)-1,4-dioxa-8-azaspiro[4.5]decane

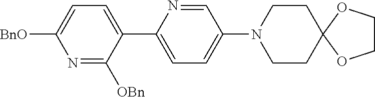 [SMILES: c1ccc(COc2ccc(-c3ccc(N4CCC5(CC4)OCCO5)cn3)c(OCc3ccccc3)n2)cc1]

To the solution of 2′,6′-bis(benzyloxy)-5-bromo-2,3′-bipyridine (2.5 g, 5.6 mmol), 1,4-dioxa-8-azaspiro[4.5]decane (1.6 g, 11.2 mmol) and CS₂CO₃ (3.6 g, 11.2 mmol) in 80 mL 1,4-dioxane, were added Pd₂(dba)₃ (1.0 g, 1.12 mmol) and XantPhos (1.0 g, 2.24 mmol). The mixture was stirred at 100° C. for 16 hours. After LCMS showed the reaction was completed, the mixture was filtered through a celite pad and washed with DCM. The filtrate was concentrated under reduced pressure to give the crude residue, which was purified by silica column chromatography (PE:EA=50:1-2:1) to afford the product (1.9 g, 66.6%). [M+H]⁺=510.5.

### Step 3: 3-(5-(1,4-dioxa-8-azaspiro[4.5]decan-8-yl)pyridin-2-yl)piperidine-2,6-dione

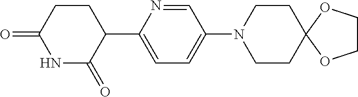 [SMILES: O=C1CCC(c2ccc(N3CCC4(CC3)OCCO4)cn2)C(=O)N1]

To the solution of 8-(2′,6′-bis(benzyloxy)-[2,3′-bipyridin]-5-yl)-1,4-dioxa-8-azaspiro[4.5]decane (1.9 g, 3.7 mmol) in 25 mL DMF and 25 mL iPrOH, Pd/C (1.2 g, 10 wt. %, wet) was added. The mixture was stirred at 50° C. for 16 hours under hydrogen atmosphere (balloon). After LCMS showed the reaction was completed, the mixture was cooled to room temperature and filtered by celite directly. The filtrate was concentrated in vacuum to afford the desired product (1.2 g, 96.7%). [M+H]⁺=332.6.

### Step 4: 3-(5-(4-oxopiperidin-1-yl)pyridin-2-yl)piperidine-2,6-dione

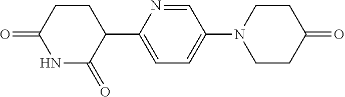 [SMILES: O=C1CCN(c2ccc(C3CCC(=O)NC3=O)nc2)CC1]

3-(5-(1,4-dioxa-8-azaspiro[4.5]decan-8-yl)pyridin-2-yl)piperidine-2,6-dione (1.2 g, 3.6 mmol) was placed in 100 mL round bottom flask with a magnetic stir bar. Then, 15 mL 8N HCl aqueous was added. The mixture was stirred at room temperature for 60 minutes. The mixture was added dropwise to sat. aq. NaHCO₃ solution and finally pH=6-7. The liquid was extracted with DCM and separated. The organic phase was concentrated in vacuum and purified with combiflash (DCM:MeOH=25:1) to afford the title compound (850 mg, 81.7% yield). [M+H]⁺=288.5.

### Step 5: 3-(5-(4-(4-(R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)piperazin-1-yl)piperidin-1-yl)pyridin-2-yl)piperidine-2,6-dione

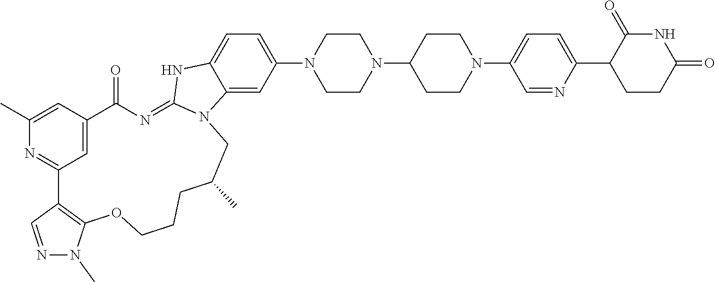 [SMILES: Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5)CC4)CC3)cc21]

The title compound was prepared in a procedure similar to that in Example 324 step 3. ¹H NMR (500 MHz, DMSO) δ_{H} 12.51 (s, 1H), 10.78 (s, 1H), 8.42 (s, 1H), 8.22 (s, 1H), 7.92 (s, 1H), 7.56 (s, 1H), 7.37 (d, J=8.7 Hz, 1H), 7.34 (d, J=8.3 Hz, 1H), 7.19-7.11 (m, 2H), 6.89 (d, J=8.8 Hz, 1H), 4.36 (d, J=4.3 Hz, 1H), 4.14 (d, J=12.0 Hz, 1H), 3.99 (d, J=8.9 Hz, 2H), 3.88 (dd, J=8.4, 5.4 Hz, 1H), 3.79 (d, J=11.8 Hz, 2H), 3.73 (s, 3H), 3.20-3.14 (m, 4H), 2.83-2.66 (m, 7H), 2.58-2.52 (m, 5H), 2.43 (s, 1H), 2.24-2.06 (m, 4H), 2.00-1.89 (m, 4H), 1.57-1.46 (m, 3H), 0.82 (d, J=6.3 Hz, 3H). [M+H]⁺=786.6.

### Example 374: (R)-3-(2,6-difluoro-4-(4-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

### Step 1: tert-butyl (R)-4-(3-((5-((4-(4-(methoxycarbonyl)-6-methylpyridin-2-yl)-1-methyl-1H-pyrazol-5-yl)oxy)-2-methylpentyl)amino)-4-nitrophenyl)-3-oxopiperazine-1-carboxylate

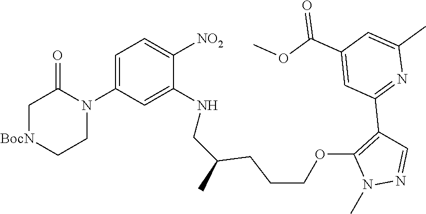 [SMILES: COC(=O)c1cc(C)nc(-c2cnn(C)c2OCCC[C@@H](C)CNc2cc(N3CCN(C(=O)OC(C)(C)C)CC3=O)ccc2[N+](=O)[O-])c1]

To a stirred solution of methyl (R)-2-(5-((5-((5-bromo-2-nitrophenyl)amino)-4-methylpentyl)oxy)-1-methyl-1H-pyrazol-4-yl)-6-methylisonicotinate (102.0 g, 186.7 mmol) and tert-butyl 3-oxopiperazine-1-carboxylate (112.1 g, 560.0 mmol) in DMSO (800 mL) were added N1,N2-dimethylethane-1,2-diamine (8.23 g, 93.3 mmol), CuI (17.8 g, 93.3 mmol) and K₃PO₄ (138.7 g, 653.3 mmol). The resulting mixture was stirred for 2 h at 100° C. under nitrogen atmosphere. The reaction solution was diluted with water, extracted with EtOAc (1000 mL×2). The combined organic layers were washed with water and brine, dried over anhydrous Na₂SO₄, filtered and concentrated under reduced pressure. The residue was purified by column chromatography (DCM:MeOH=100/0 to 100/4) to afford the product (100.0 g, 80.4% yield). [M+H]⁺=666.7.

### Step 2: tert-butyl (R)-4-(4-amino-3-((5-((4-(4-(methoxycarbonyl)-6-methylpyridin-2-yl)-1-methyl-1H-pyrazol-5-yl)oxy-2-methylpentyl)amino)phenyl)-3-oxopiperazine-1-carboxylate

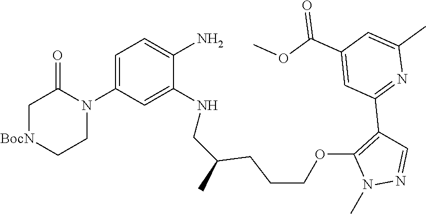 [SMILES: COC(=O)c1cc(C)nc(-c2cnn(C)c2OCCC[C@@H](C)CNc2cc(N3CCN(C(=O)OC(C)(C)C)CC3=O)ccc2N)c1]

A mixture of tert-butyl (R)-4-(3-((5-((4-(4-(methoxycarbonyl)-6-methylpyridin-2-yl)-1-methyl-1H-pyrazol-5-yl)oxy)-2-methylpentyl)amino)-4-nitrophenyl)-3-oxopiperazine-1-carboxylate (45.0 g, 67.6 mmol) and Raney-Ni (15.0 g) in THF (450 mL) was degassed and purged with H₂(40 Psi) for 3 times at 30° C., and then the mixture was stirred at 30° C. for 3 hours under H₂ atmosphere. The mixture was filtered and concentrated under reduced pressure. The product (41.0 g, 64.5 mmol, 95.4% yield) was used for next step without purification.[M+H]⁺=636.7.

### Step 3: tert-butyl (R)-4-(2-amino-1-(5 (4-(4-(methoxycarbonyl)-6-methylpyridin-2-yl)-1-methyl-1H-pyrazol-5-yl)oxy)-2-methylpentyl)-1H-benzo[d]imidazol-6-yl)-3-oxopiperazine-1-carboxylate

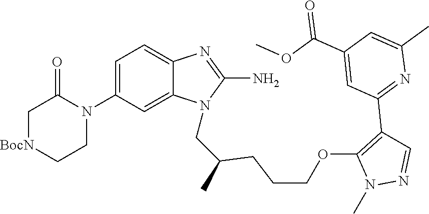 [SMILES: COC(=O)c1cc(C)nc(-c2cnn(C)c2OCCC[C@@H](C)Cn2c(N)nc3ccc(N4CCN(C(=O)OC(C)(C)C)CC4=O)cc32)c1]

To a solution of compound tert-butyl (R)-4-(4-amino-3-((5-((4-(4-(methoxycarbonyl)-6-methylpyridin-2-yl)-1-methyl-1H-pyrazol-5-yl)ox y)-2-methylpentyl)amino)phenyl)-3-oxopiperazine-1-carboxylate (41.0 g, 64.5 mmol) in DCM (250 mL) and t-BuOH (50 mL) was added CNBr (10.25 g, 96.7 mmol) at 25° C. The mixture was stirred at 25° C. for 24 hours. Then the reaction mixture was quenched by aqueous NaHCO₃ solution (60 mL) for 10 minutes. The layers were separated and the organic layer was washed with saturated aqueous NaHCO₃ solution (200 mL*2). The organic layer was dried over Na₂SO₄, filtered and concentrated under reduced pressure to give a product (42.0 g, 63.6 mmol, 98.6% yield) which was used in the next step without further purification. [M+H]⁺=661.7

### Step 4: (R)-2-(5-((5-(2-amino-6-(4-(tert-butoxycarbonyl)-2-oxopiperazin-1-yl)-1H-benzo[d]imidazol-1-yl)-4-methylpentyloxy)-1-methyl-1H-pyrazol-4-yl)-6-methylisonicotinic acid

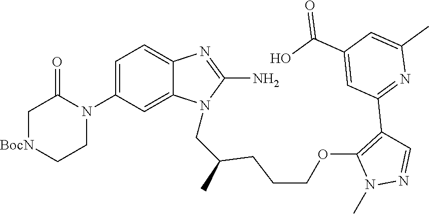 [SMILES: Cc1cc(C(=O)O)cc(-c2cnn(C)c2OCCC[C@@H](C)Cn2c(N)nc3ccc(N4CCN(C(=O)OC(C)(C)C)CC4=O)cc32)n1]

To a solution of tert-butyl (R)-4-(2-amino-1-(5-((4-(4-(methoxycarbonyl)-6-methylpyridin-2-yl)-1-methyl-1H-pyrazol-5-yl)oxy)-2-methylpentyl)-1H-benzo[d]imidazol-6-yl)-3-oxopiperazine-1-carboxylate (42.0 g, 63.6 mmol) in THF (200 mL) was added NaOH (12.7 g, 317.8 mmol) in H₂O (200 mL) at 25° C. The mixture was stirred at 25° C. for 2 hours. Then the reaction mixture was concentrated under reduced pressure to remove solvent. The residue was acidified with 4M aqueous HCl to pH=6, resulting in the formation of a slurry. The solid was filtered, washed with water (30 mL) and dried under reduced pressure to give a product. The product (38.0 g, crude) was used for next step without purification. [M+H]⁺=647.7.

### Step 5: tert-butyl (R,E)-3-oxo-4-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5′-yl)piperazine-1-carboxylate

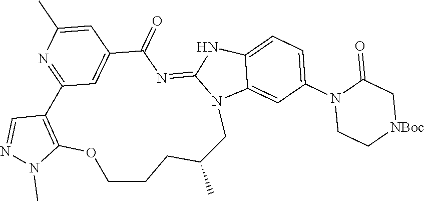 [SMILES: Cc1cc2cc(n1)-c1cnn(C)c1OCCC[C@@H](C)CN1/C(=N/C2=O)Nc2ccc(N3CCN(C(=O)OC(C)(C)C)CC3=O)cc21]

To a solution of (R)-2-(5-((5-(2-amino-6-(4-(tert-butoxycarbonyl)-2-oxopiperazin-1-yl)-IH-benzo[d]imidazol-1-yl)-4-methylpentyl)oxy)-1-methyl-1H-pyrazol-4-yl)-6-methylisonicotinic acid (38.0 g, 58.8 mmol) in DCM (380 mL) was added HATU (26.8 g, 70.5 mmol) and DIEA (15.2 g, 117.5 mmol) at 25° C. The mixture was stirred at 25° C. for 1 hour and turned black solution. The reaction mixture was washed with saturated NaHCO₃ solution (100 mL*3), dried over Na₂SO₄, filtered and concentrated under reduced pressure to give a residue. The residue was purified by column chromatography (DCM:MeOH=100/1 to 10/1) to afford the product (19.0 g, 51.4% yield). [M+H]⁺=629.7.

### Step 6: (R,E)-1¹,2⁶,7-trimethyl-5⁶42-oxopiperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

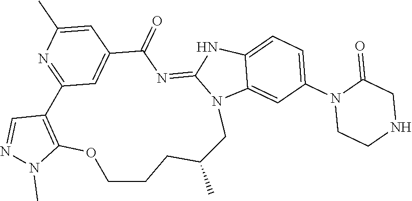 [SMILES: Cc1cc2cc(n1)-c1cnn(C)c1OCCC[C@@H](C)CN1/C(=N/C2=O)Nc2ccc(N3CCNCC3=O)cc21]

To a solution of tert-butyl (R,E)-3-oxo-4-(1¹,2⁶,7-trimethyl-3-oxo-5²,5³-dihydro-1¹H,5¹H-11-oxa-4-aza-5(2,1)-benzo[d]imidazo 1a-2(2,4)-pyridina-1(4,5)-pyrazolacycloundecaphane-5⁶-yl)piperazine-1-carboxylate (19.0 g, 30.2 mmol) in DCM (190 mL) was added TFA (40 mL). The reaction solution was stirred at r.t for 1 hrs and then concentrated in vacuo. The residue was dissolved in DCM and then washed with sat NaHCO₃ solution (100 mL) and brine (100 mL), dried over Na₂SO₄, filtered and concentrated in vacuum to afford (R,E)-1¹,2⁶,7-trimethyl-5⁶-(2-oxopiperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (13.9 g, 87.0%). [M+H]⁺=529.7.

### Step 7: (R)-3-(2,6-difluoro-4-(4-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

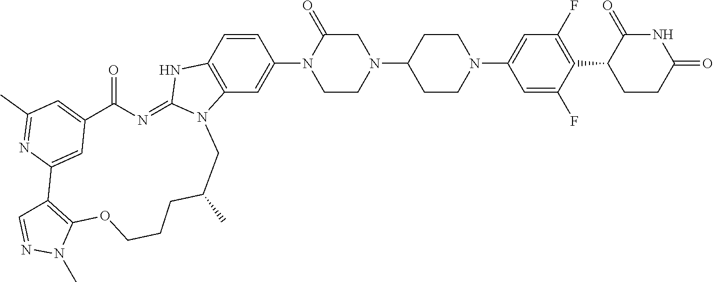 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3=O)cc21]

To a solution of (R,E)-1¹,2′,7-trimethyl-5⁶-(2-oxopiperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (10.0 g, 18.9 mmol), (R)-3-(2,6-difluoro-4-(4-oxopiperidin-1-yl)phenyl)piperidine-2,6-dione (7.9 g, 24.6 mmol), NaBH(OAc)₃ (8.0 g, 37.8 mmol) and DCM (300 mL). After stirring for 4 h at 50° C., the reaction mixture was concentrated under reduced pressure. Purification by flash column chromatography (0-70% MeOH in DCM) to afford the title compound (11.0 g, 69.6%). ¹H NMR (500 MHz, DMSO) δ 12.76 (s, 1H), 10.87 (s, 1H), 8.43 (s, 1H), 7.92 (s, 1H), 7.65 (d, J=1.2 Hz, 1H), 7.57 (s, 1H), 7.52 (d, J=8.4 Hz, 1H), 7.16 (dd, J=8.5, 1.7 Hz, 1H), 6.67 (s, 1H), 6.65 (s, 1H), 4.36 (d, J=4.8 Hz, 1H), 4.18 (d, J=10.9 Hz, 1H), 4.05 (dd, J=12.6, 5.0 Hz, 1H), 4.03-3.97 (m, 1H), 3.94 (dd, J=13.1, 10.4 Hz, 1H), 3.82 (d, J=12.4 Hz, 2H), 3.73 (s, 3H), 3.70 (dd, J=11.1, 5.7 Hz, 1H), 3.67-3.61 (m, 1H), 3.33 (s, 2H), 2.91 (t, J=5.1 Hz, 2H), 2.86-2.74 (m, 4H), 2.56 (s, 3H), 2.55-2.52 (m, 2H), 2.27-2.16 (m, 1H), 2.15-2.04 (m, 1H), 2.01-1.86 (m, 5H), 1.57-1.39 (m, 3H), 0.82 (d, J=6.5 Hz, 3H). [M+H]⁺=835.7.

### Example 379: (R,E)-N-(1-(2-(1-((S)-2,6-dioxopiperidin-3-yl)-3,3-dimethyl-2-oxoindolin-4-yl)ethyl)piperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

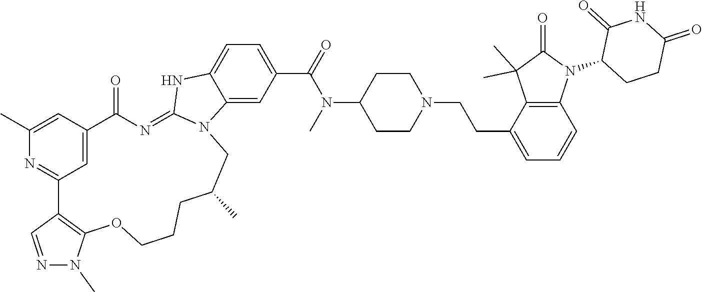 [SMILES: Cc1cc2cc(n1)-c1cnn(C)c1OCCC[C@@H](C)CN1/C(=N/C2=O)Nc2ccc(C(=O)N(C)C3CCN(CCc4cccc5c4C(C)(C)C(=O)N5[C@H]4CCC(=O)NC4=O)CC3)cc21]

The titled compound was prepared in a manner similar to that in Example 260.

¹H NMR (500 MHz, DMSO-d₆) δ 12.88 (s, 1H), 11.07 (s, 1H), 8.43 (s, 1H), 7.93 (s, 1H), 7.72 (s, IH), 7.59 (s, 2H), 7.23 (s, 2H), 6.88 (s, 2H), 5.21 (s, 1H), 4.41-4.33 (m, 1H), 4.27-4.16 (m, 1H), 4.10-3.98 (m, 2H), 3.76 (s, 3H), 2.95-2.78 (m, 8H), 2.60 (s, 3H), 2.56 (s, 3H), 2.17 (s, 2H), 2.05 (s, 1H), 2.04-1.87 (m, 5H), 1.79-1.81 (m, 1H), 1.75 (s, 1H), 1.49-1.29 (m, 8H), 1.24 (d, J=28.4 Hz, 2H), 0.85 (s, 3H). [M+H]⁺=869.4

### Example 380: (R)-3-(4-(4-((2S,5R)-2,5-dimethyl-4-((R,E)-1¹,2⁶,7-trimethyl-3-oxo-5²,5¹-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)-2,6-difluorophenyl)piperidine-2,6-dione

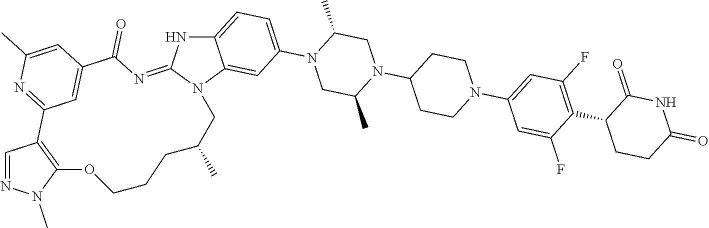 [SMILES: Cc1cc2cc(n1)-c1cnn(C)c1OCCC[C@@H](C)CN1/C(=N/C2=O)Nc2ccc(N3C[C@H](C)N(C4CCN(c5cc(F)c([C@H]6CCC(=O)NC6=O)c(F)c5)CC4)C[C@H]3C)cc21]

The titled compound was prepared in a manner similar to that in Example 324.

¹H NMR (500 MHz, DMSO) δ 12.74 (s, 1H), 10.89 (s, 1H), 8.47 (s, 1H), 7.99 (s, 1H), 7.63 (s, 1H), 7.52 (d, J=8.5 Hz, 1H), 7.35 (s, 1H), 7.06 (d, J=8.1 Hz, 1H), 6.73 (s, 1H), 6.70 (s, 1H), 4.38 (s, 2H), 4.00 (d, J=10.7 Hz, 6H), 3.74 (s, 3H), 2.98 (d, J=12.0 Hz, 4H), 2.79 (s, 6H), 2.59 (s, 3H), 2.22 (s, 2H), 2.08 (s, 2H), 2.00-1.91 (m, 6H), 1.35 (d, J=6.1 Hz, 3H), 1.09 (d, J=6.5 Hz, 1H), 0.92 (d, J=5.9 Hz, 3H), 0.81 (d, J=6.3 Hz, 3H). [M+H]⁺=849.4

### Example 381: (R)-3-(2,6-difluoro-4-((1′-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-[1,4′-bipiperidin]-4-yl) oxy)phenyl)piperidine-2,6-dione

### Step 1: (R,E) 1¹,2⁶,7-trimethyl-5⁶-(1,4-dioxa-8-azaspiro[4.5]decan-8-yl)-5²,5¹-dihydro-1¹H,5¹H-11-oxa-4-aza-5(2,1)-benzo[d]imidazola-2(2,4)-pyridina-1(4,5)-pyrazolacycloundecaphan-3-one

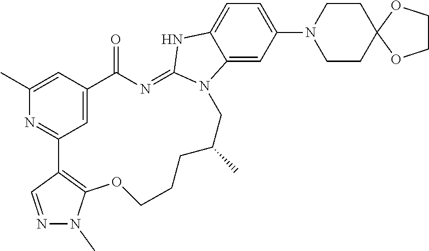 [SMILES: Cc1cc2cc(n1)-c1cnn(C)c1OCCC[C@@H](C)CN1/C(=N/C2=O)Nc2ccc(N3CCC4(CC3)OCCO4)cc21]

To the solution of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (3.0 g, 5.9 mmol), 1,4-dioxa-8-azaspiro[4.5]decane (1.7 g, 11.8 mmol) and t-BuONa (1.2 g, 11.8 mmol) in 80 mL DMA, were added Pd₂(dba)₃ (540 mg, 0.59 mmol) and RuPhos (550 mg, 1.18 mmol). The mixture was stirred at 100° C. for 3 hours under N₂. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (DCM:MeOH=100:1-15:1) to afford the product (2.9 g, 85.8%). [M+H]⁺=572.5.

### Step 2: (R,E)-1¹,2⁶,7-trimethyl-5⁶44-oxopiperidin-1-yl-5²,5³-dihydro-1¹H,5¹H-11-oxa-4-aza-5(2,1)-benzo[d]imidazola-2(2,4)-pyridina-1(4,5)-pyrazolacycloundecaphan-3-one

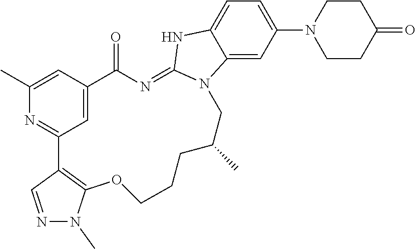 [SMILES: Cc1cc2cc(n1)-c1cnn(C)c1OCCC[C@@H](C)CN1/C(=N/C2=O)Nc2ccc(N3CCC(=O)CC3)cc21]

(R,E)-1¹,2⁶,7-trimethyl-5⁶-(1,4-dioxa-8-azaspiro[4.5]decan-8-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (2.9 g, 5.1 mmol) was placed in round bottom flask. Then, 45 mL 8N HCl aqueous was added. The mixture was stirred at room temperature for 2 hours. The mixture was added dropwise to sat. aq. NaHCO₃ solution and finally pH=6-7. The liquid was extracted with DCM and separated. The organic phase was concentrated in vacuum and purified with combiflash (DCM:MeOH=20:1) to afford the title compound (2.5 g, 93.6% yield). [M+H]⁺=528.5.

### Step 3: (R)-3-(2,6-difluoro-4-((1′-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-[1,4′-bipiperidin]-4-yl) oxy)phenyl)piperidine-2,6-dione

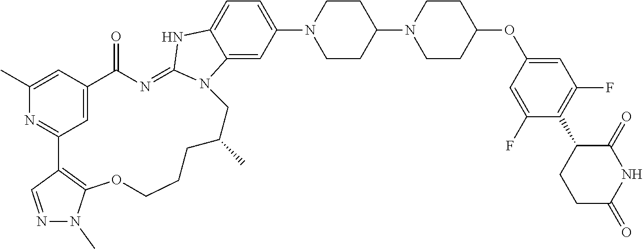 [SMILES: Cc1cc2cc(n1)-c1cnn(C)c1OCCC[C@@H](C)CN1/C(=N/C2=O)Nc2ccc(N3CCC(N4CCC(Oc5cc(F)c([C@H]6CCC(=O)NC6=O)c(F)c5)CC4)CC3)cc21]

To a solution of (R,E)-1¹,2⁶,7-trimethyl-5⁶-(4-oxopiperidin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 0.19 mmol), (R)-3-(2,6-difluoro-4-(piperidin-4-yloxy)phenyl)piperidine-2,6-dione hydrochloride (90 mg, 0.25 mmol) in DCE (8 mL) was added STAB (121 mg, 0.57 mmol). Then the mixture was stirred at 50° C. for 16 hours. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=10:1), followed by Prep-HPLC chromatography to afford the title product (48 mg, 30.4%). ¹H NMR (500 MHz, DMSO) δ_{H} 12.50 (s, 1H), 10.92 (s, 1H), 8.42 (s, 1H), 7.92 (s, 1H), 7.55 (s, 1H), 7.36 (d, J=8.8 Hz, 1H), 7.14 (s, 1H), 6.90 (d, J=8.7 Hz, 1H), 6.77 (d, J=10.9 Hz, 2H), 4.45-4.33 (m, 2H), 4.13 (d, J=12.1 Hz, 2H), 4.01-3.95 (m, 2H), 3.73 (s, 3H), 2.85-2.75 (m, 5H), 2.68 (t, J=11.6 Hz, 3H), 2.55 (s, 3H), 2.44 (d, J=9.6 Hz, 3H), 2.21 (s, 1H), 2.11 (d, J=12.6 Hz, 1H), 2.02-1.82 (m, 8H), 1.60 (d, J=9.0 Hz, 4H), 1.45 (s, 1H), 0.82 (d, J=6.3 Hz, 3H). [M+H]⁺=836.6.

### Example 382: (R,E)-N—((3S,4R)-1-(1-(4-((R)-2,6-dioxopiperidin-3-yl)-3,5-difluorophenyl)azetidin-3-yl)-3-fluoropiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁵-carboxamide

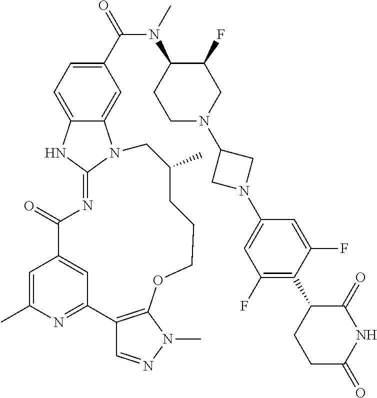 [SMILES: Cc1cc2cc(n1)-c1cnn(C)c1OCCC[C@@H](C)CN1/C(=N/C2=O)Nc2ccc(C(=O)N(C)[C@@H]3CCN(C4CN(c5cc(F)c([C@H]6CCC(=O)NC6=O)c(F)c5)C4)C[C@@H]3F)cc21]

The titled compound was prepared in a manner similar to that in Example 260.

¹H NMR (500 MHz, DMSO) δ 12.87 (s, 1H), 10.85 (s, 1H), 8.43 (s, 1H), 7.92 (s, 1H), 7.73 (s, 1H), 7.57 (t, J=4.0 Hz, 2H), 7.27 (s, 1H), 6.12 (s, 2H), 5.05-4.94 (m, 1H), 4.36 (d, J=4.2 Hz, 1H), 4.19 (d, J=12.3 Hz, 1H), 4.02 (d, J=13.1 Hz, 3H), 3.93 (s, 2H), 3.73 (s, 3H), 3.61 (s, 2H), 3.37 (s, 1H), 3.31-3.27 (m, 2H), 3.09 (s, 1H), 2.94 (s, 4H), 2.84-2.73 (m, 2H), 2.56 (s, 3H), 2.51 (s, 2H), 2.21 (s, 2H), 2.07 (d, J=13.3 Hz, 1H), 1.97 (d, J=17.0 Hz, 3H), 1.73 (s, 1H), 1.44 (s, 1H), 0.82 (d, J=6.3 Hz, 3H). [M+H]⁺=867.7

### Example 383: (R)-3-(2,6-difluoro-4-(4-((S)-3-methyl-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)piperazin-1-yl)piperidin-1-yl)phenyl)piperidine-2,6-dione

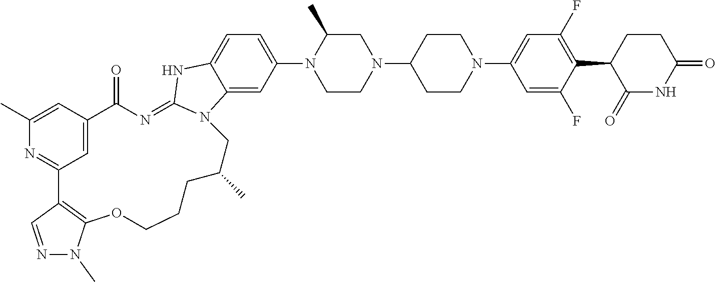 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)C[C@@H]3C)cc21]

The titled compound was prepared in a manner similar to that in Example 324.

¹H NMR (500 MHz, DMSO) δ 12.51 (s, 1H), 10.87 (s, 1H), 8.42 (s, 1H), 7.92 (s, 1H), 7.56 (s, 1H), 7.38 (d, J=8.6 Hz, 1H), 7.15 (s, 1H), 6.86 (d, J=8.4 Hz, 1H), 6.64 (d, J=12.8 Hz, 2H), 4.39-4.33 (m, 1H), 4.15-4.13 (m, 1H), 4.06-3.94 (m, 3H), 3.89 (s, 1H), 3.80 (d, J=10.9 Hz, 2H), 3.73 (s, 3H), 3.20 (d, J=11.0 Hz, 1H), 2.99 (s, 1H), 2.87-2.75 (m, 5H), 2.69-2.60 (m, 3H), 2.55 (s, 3H), 2.47-2.42 (m, 2H), 2.22 (s, 2H), 2.11-2.06 (m, 1H), 1.97 (s, 2H), 1.85 (s, 2H), 1.57-1.42 (m, 3H), 0.94 (d, J=6.1 Hz, 3H), 0.81 (d, J=6.3 Hz, 3H); [M+H]⁺=835.7.

### Example 384: (R)-3-(2,6-difluoro-4-(4-((R)-3-methyl-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

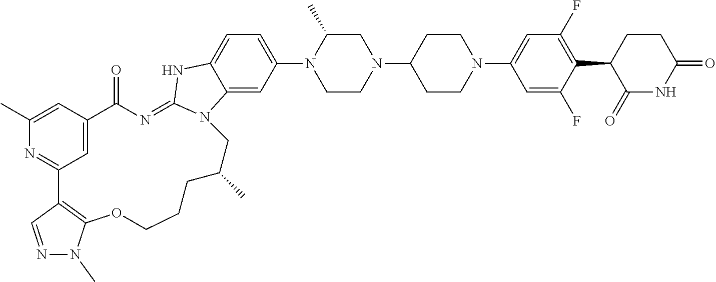 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)C[C@H]3C)cc21]

The titled compound was prepared in a manner similar to that in Example 324.

¹H NMR (500 MHz, DMSO) δ 12.50 (s, 1H), 10.87 (s, 1H), 8.42 (s, 1H), 7.92 (s, 1H), 7.55 (s, 1H), 7.37 (d, J=8.7 Hz, 1H), 7.13 (s, 1H), 6.88 (d, J=8.8 Hz, 1H), 6.64 (d, J=12.9 Hz, 2H), 4.36 (d, J=4.2 Hz, 1H), 4.15 (d, J=11.9 Hz, 1H), 4.05 (dd, J=12.5, 5.1 Hz, 1H), 4.01-3.94 (m, 2H), 3.90 (s, IH), 3.80 (d, J=12.4 Hz, 2H), 3.73 (s, 3H), 3.22 (d, J=11.2 Hz, 1H), 2.99 (s, 1H), 2.90-2.84 (m, 1H), 2.82-2.77 (m, 4H), 2.67-2.59 (m, 3H), 2.55 (s, 3H), 2.46-2.43 (m, 2H), 2.21 (s, 2H), 2.08 (s, 1H), 1.97-1.94 (m, 2H), 1.85 (s, 2H), 1.53-1.42 (m, 3H), 0.95 (d, J=6.2 Hz, 3H), 0.81 (d, J=6.3 Hz, 3H); [M+H]⁺=835.7.

### Example 385: (R)-3-(2,6-difluoro-4-(4-((R)-3-(methoxymethyl)-4-((R,E)-1¹,2⁶,7-trimethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

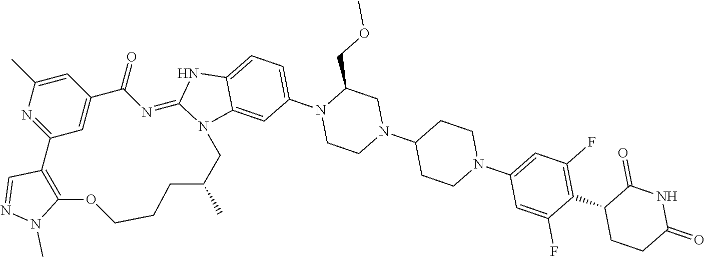 [SMILES: COC[C@H]1CN(C2CCN(c3cc(F)c([C@H]4CCC(=O)NC4=O)c(F)c3)CC2)CCN1c1ccc2c(c1)N1C[C@H](C)CCCOc3c(cnn3C)-c3cc(cc(C)n3)C(=O)/N=C/1N2]

The titled compound was prepared in a manner similar to that in Example 324.

¹H NMR (500 MHz, DMSO) δ 12.76-12.59 (m, 1H), 10.89 (s, 1H), 8.46 (s, 1H), 7.97 (s, 1H), 7.61 (s, 1H), 7.56-7.39 (m, 1H), 7.29-7.21(m, 1H), 7.10 (s, 1H), 6.92-6.74 (m, 2H), 4.46-4.38 (m, 1H), 4.24-4.13 (m, 1H), 4.11-3.95 (m, 5H), 3.74 (s, 3H), 3.71-3.54 (m, 6H), 3.34-3.25 (m, 4H), 3.22 (s, 2H), 3.14-3.11 (m, 2H), 2.92-2.74 (m, 4H), 2.58 (s, 3H), 2.40 (t, J=6.0 Hz, 1H), 2.28-2.15 (m, 2H), 2.10 (d, J=13.2 Hz, 1H), 2.04-1.89 (m, 3H), 1.81-1.62 (m, 2H), 1.50-1.41 (m, 1H), 0.82 (d, J=6.3 Hz, 3H). [M+H]⁺=865.4

### Example 386: (R)-3-(2,6-difluoro-4-(4-((S)-3-(methoxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)piperazin-1-yl)piperidin-1-yl)phenyl)piperidine-2,6-dione

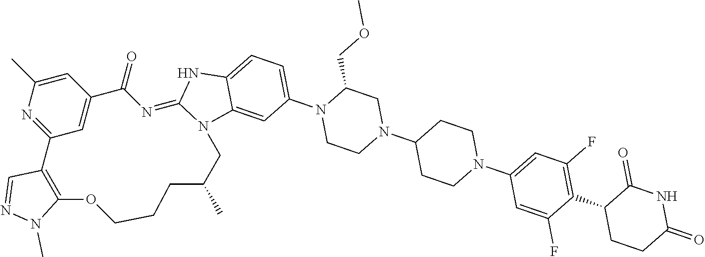 [SMILES: COC[C@@H]1CN(C2CCN(c3cc(F)c([C@H]4CCC(=O)NC4=O)c(F)c3)CC2)CCN1c1ccc2c(c1)N1C[C@H](C)CCCOc3c(cnn3C)-c3cc(cc(C)n3)C(=O)/N=C/1N2]

The titled compound was prepared in a manner similar to that in Example 324.

¹H NMR (500 MHz, DMSO) δ 12.76-12.59 (m, 1H), 10.89 (s, 1H), 8.46 (s, 1H), 7.97 (s, 1H), 7.61 (s, 1H), 7.56-7.39 (m, 1H), 7.29-7.21(m, 1H), 7.10 (s, 1H), 6.92-6.74 (m, 2H), 4.46-4.38 (m, 1H), 4.24-4.13 (m, 1H), 4.11-3.95 (m, 5H), 3.74 (s, 3H), 3.71-3.54 (m, 6H), 3.34-3.25 (m, 4H), 3.22 (s, 2H), 3.14-3.11 (m, 2H), 2.92-2.74 (m, 4H), 2.58 (s, 3H), 2.40 (t, J=6.0 Hz, 1H), 2.28-2.15 (m, 2H), 2.10 (d, J=13.2 Hz, 1H), 2.04-1.89 (m, 3H), 1.81-1.62 (m, 2H), 1.50-1.41 (m, 1H), 0.82 (d, J=6.3 Hz, 3H). [M+H]⁺=865.4

### Example 387: (R)-3-(2,6-difluoro-4-(4-(1-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)piperazin-1-yl)phenyl)piperidine-2,6-dione

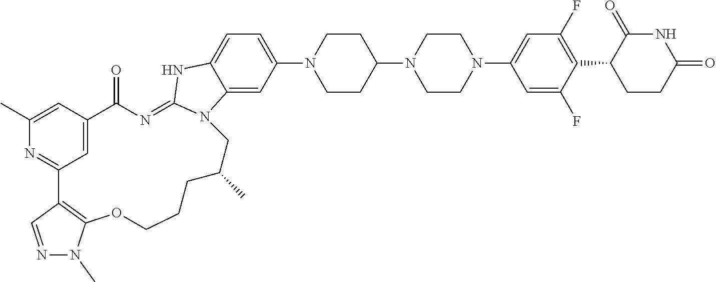 [SMILES: Cc1cc2cc(n1)-c1cnn(C)c1OCCC[C@@H](C)CN1/C(=N/C2=O)Nc2ccc(N3CCC(N4CCN(c5cc(F)c([C@H]6CCC(=O)NC6=O)c(F)c5)CC4)CC3)cc21]

The title compound was prepared in a procedure similar to that in Example 381 step 3. ¹H NMR (500 MHz, DMSO) δ_{H} 12.50 (s, 1H), 10.87 (s, 1H), 8.42 (s, 1H), 7.92(s, 1H), 7.56 (s, 1H), 7.36 (s, 1H), 7.15 (s, 1H), 6.90 (d, J=8.6 Hz, 1H), 6.64 (d, J=12.7 Hz, 2H), 4.36 (d, J=4.5 Hz, 1H),4.14 (d, J=11.7 Hz, 1H), 4.07-3.97 (m, 3H), 3.73 (s, 4H), 3.22-3.15 (m, 4H), 2.85-2.68 (m, 6H), 2.65-2.60 (m, 4H), 2.55 (s, 3H), 2.40 (s, 1H), 2.21 (s, 1H), 2.14-2.05 (m, 1H), 2.00-1.87 (m, 5H), 1.59 (d, J=11.5 Hz, 2H), 1.45 (s, 1H), 0.82 (d, J=6.4 Hz, 3H). [M+H]⁺=821.6.

### Example 388: (R)-3-(2,6-difluoro-4-(4-((R)-2-methyl-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-yl)phenyl)piperidine-2,6-dione

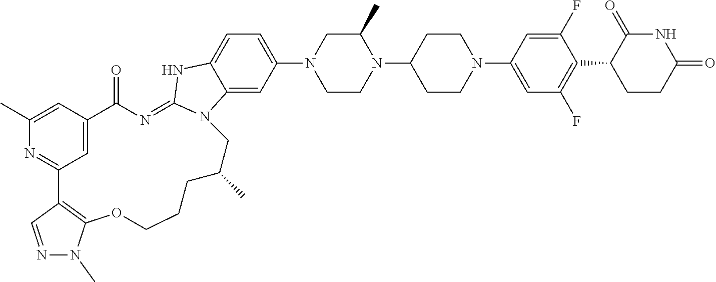 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@H](C)C3)cc21]

The titled compound was prepared in a manner similar to that in Example 324. ¹H NMR (500 MHz, DMSO) δ 12.50 (s, 1H), 10.87 (s, 1H), 8.42 (s, 1H), 7.92 (s, 1H), 7.55 (s, 1H), 7.36 (d, J=8.5 Hz, 1H), 7.12 (s, 1H), 6.87 (d, J=8.5 Hz, 1H), 6.64 (d, J=13.0 Hz, 2H), 4.40-4.34 (m, 1H), 4.15-4.13 (m, 1H), 4.07-3.95 (m, 4H), 3.86-3.83 (m, 2H), 3.73 (s, 3H), 3.47-3.43 (m, 2H), 2.97-2.89 (m, 2H), 2.87-2.73 (m, 3H), 2.64-2.59 (m, 2H), 2.54 (s, 3H), 2.27-2.18 (m, 2H), 2.17-2.12 (m, 2H), 2.01-1.87 (m, 3H), 1.80-1.62 (m, 4H), 1.51-1.37 (m, 2H), 1.13 (d, J=6.0 Hz, 3H), 0.81 (d, J=6.5 Hz, 3H), [M+H]⁺=835.6.

### Example 390: (R)-3-(2,6-difluoro-4-(4-(4-((R,E)-5⁵-(methoxymethyl)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

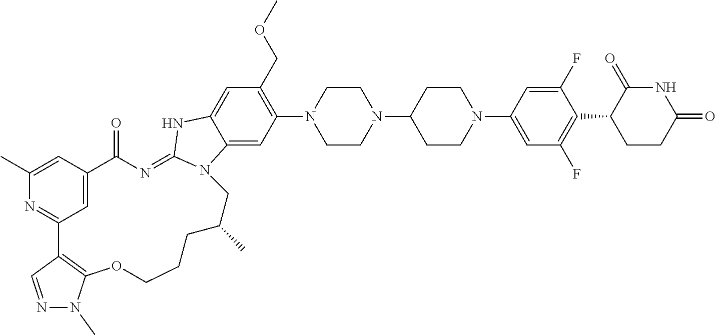 [SMILES: COCc1cc2c(cc1N1CCN(C3CCN(c4cc(F)c([C@H]5CCC(=O)NC5=O)c(F)c4)CC3)CC1)N1C[C@H](C)CCCOc3c(cnn3C)-c3cc(cc(C)n3)C(=O)/N=C/1N2]

The titled compound was prepared in a manner similar to that in Example 324.

¹H NMR (500 MHz, DMSO) δ 12.62 (s, 1H), 10.87 (s, 1H), 8.42 (s, 1H), 7.92 (s, 1H), 7.56 (s, 1H), 7.53 (s, 1H), 7.41 (s, 1H), 6.66 (s, 1H), 6.63 (s, 1H), 4.52 (s, 1H), 4.39-4.35 (m, 1H), 4.17 (d, J=11.0 Hz, 1H), 4.08-4.03 (m, 1H), 3.99-3.96 (m, 2H), 3.81 (d, J=11.1 Hz, 2H), 3.73 (s, 3H), 2.90 (s, 3H), 2.77 (d, J=12.0 Hz, 4H), 2.69 (s, 3H), 2.58-2.53 (m, 3H), 2.19-2.16 (m, 2H), 1.99-1.96 (m, 2H), 1.89-1.84 (m, 1H), 1.49-1.45 (m, 3H), 1.26-1.21 (m, 9H), 0.87-0.83 (m, 1H), 0.81 (d, J=6.5 Hz 3H); [M+H]⁺=865.7.

### Example 391: (R)-3-(4-(4-(4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-2,6-difluorophenyl)piperidine-2,6-dione

### Step 1: (but-3-en-1-yloxy)(tert-butyl)diphenylsilane

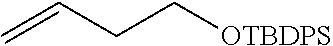

A 500 mL round bottom flask equipped with a magnetic stirrer, was charged with but-3-en-1-ol (5.0 g, 69.4 mmol), TBDPSCl (28.65 g, 104.6 mmol), imidazole (10.39 g, 152.7 mmol), DMF (80 mL). The resulting mixture was degassed under reduced pressure and purged with N₂ for three times, then stirred at rt for overnight. The mixture was diluted with EA (200 mL), washed with water (2×100 mL), brine (100 mL), dried and concentrated. The residue was purified by column chromatography (pure PE to PE/EA=10:1) to afford crude product (15 g, 48.2 mmol, 69.4%). ¹H NMR (300 MHz, DMSO-d₆) 5 ppm 7.68-7.58 (m, 4H), 7.53-7.37 (m, 6H), 5.82 (ddt, J=17.1, 10.2, 6.8 Hz, 1H), 5.13-4.97 (m, 2H), 3.69 (t, J=6.6 Hz, 2H), 2.28 (qt, J=6.6, 1.4 Hz, 2H), 0.99 (s, 9H).

### Step 2: ethyl 2-(2-((tert-butyldiphenylsilyl)oxy)ethyl)cyclopropane-1-carboxylate

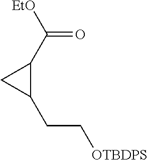

A three-neck round bottom flask equipped with a magnetic stirrer, was charged with (but-3-en-1-yloxy)(tert-butyl)diphenylsilane (10 g, 32.2 mmol), Rh₂(OAc)₄ (1.42 g, 3.22 mmol), DCM (200 ml). The mixture was degassed under reduced pressure and purged with N₂ for three times. Ethyl 2-diazoacetate (18.34 g, 160.7 mmol) in DCM (20 ml) was injected into the flask, the flask was degassed and purged with N₂ once more, then the resulting mixture was stirred at rt for overnight. The mixture was diluted with EA, washed with water and brine, dried, concentrated. The residue was purified by column chromatography (pure PE to PE/EA=20:1) to afford crude product (6 g, 15.1 mmol, 46.8%). [M+H]⁺=397.5

### Step 3: (2-424(tert-butyldiphenylsilyl)oxy)ethyl)cyclopropyl)methanol

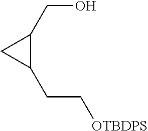

A round bottom flask equipped with a magnetic stirrer, was charged with ethyl 2-(2-((tert-butyldiphenylsilyl)oxy)ethyl)cyclopropane-1-carboxylate (4.0 g, 10.1 mmol), THF (70 mL). The temperature was lowered to 0° C. LiAlH₄ (1.15 g, 30.3 mmol) was added portionwise into the mixture while maintaining temperature at 0° C. The resulting mixture was stirred at rt for 2 hrs. The mixture was quenched with sodium sulfatedecahydrate at 0° C. for 30 minutes. Then diluted with DCM/MEOH (50 ml/50 ml), concentrated. The residue was purified by column chromatography (PE/EA=10:1 to 6:1 to afford product (730 mg, 2.05 mmol, 20.8%). [M+H]⁺=355.5.

### Step 4: 2-((2-(2-((tert-butyldiphenylsilyl)oxy)ethyl)cyclopropyl)methyl)isoindoline-1,3-dione

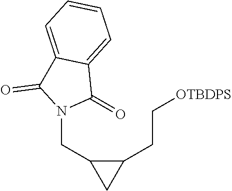

A round bottom flask equipped with a magnetic stirrer, was charged with (2-(2-((tert-butyldiphenylsilyl)oxy)ethyl)cyclopropyl)methanol (680 mg, 1.91 mmol), isoindoline-1,3-dione (564 mg, 3.82 mmol), PPh₃ (1.01 g, 3.82 mmol), and THF (10 ml). The mixture was degassed under reduced pressure and purged with N₂ for three times. DIAD (774.6 mg, 3.82 mmol) was injected into the flask, the flask was degassed and purged with N2 once more, Then the resulting mixture was stirred at rt for 2 hrs. The mixture was diluted with EA, washed with water and brine, dried, concentrated. The residue was purified by column chromatography (PE/EA=10:1 to 7:1) to afford product (850 mg, 1.76 mmol, 85.4%). [M+H]⁺=484.5.

### Step 5: 2-(((1R,2S)-2-(2-((tert-butyldiphenylsilyl)oxy)ethyl)cyclopropyl)methyl)isoindoline-1,3-dione

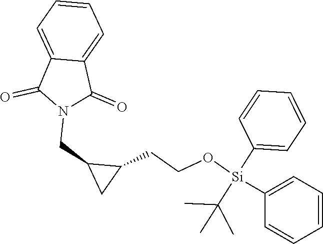 [SMILES: CC(C)(C)[Si](OCC[C@@H]1C[C@H]1CN1C(=O)c2ccccc2C1=O)(c1ccccc1)c1ccccc1]

The crude product (20 g) was purified by Prep-SFC with the following conditions (5% to 20% in 2 min, hold 1 min at 20%, IPA (0.1% DEA)) to afford 2-(((1R,2S)-2-(2-((tert-butyldiphenylsilyl)oxy)ethyl)cyclopropyl)methyl)isoindoline-1,3-dione (8.12 g, 40.6%, ee=98.58%). ¹H NMR (300 MHz, DMSO-d₆) δ ppm 7.84 (s, 4H), 7.52 (td, J=8.1, 1.6 Hz, 4H), 7.45-7.36 (m, 6H), 3.61-3.47 (m, 3H), 3.35 (dd, J=14.2, 7.5 Hz. IH), 1.52 (dq, J=12.6, 6.4 Hz, 1H), 1.32-1.19 (m, 1H), 0.98 (d, J=7.1 Hz, H), 0.92 (s, 9H), 0.84 (s, 2H), 0.49 (dt, J=8.8, 4.6 Hz, 1H), 0.30 (dt, J=9.6, 5.0 Hz, 1H). [M+H]⁺=484.5.

### Step 6: 2-(((1S,2R)-2-(2-((tert-butyldiphenylsilyl)oxy)ethyl)cyclopropyl)methyl)isoindoline-1,3-dione

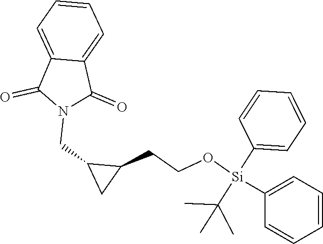 [SMILES: CC(C)(C)[Si](OCC[C@H]1C[C@@H]1CN1C(=O)c2ccccc2C1=O)(c1ccccc1)c1ccccc1]

The crude product (20 g) was purified by Prep-SFC with the following conditions (5% to 20% in 2 min, hold 1 min at 20%, IPA (0.1% DEA)) to afford 2-((1S,2R)-2-(2-((tert-butyldiphenylsilyl)oxy)ethyl)cyclopropyl)methyl)isoindoline-1,3-dione (9.98 g, 49.9%, ee=99.56%). ¹H NMR (300 MHz, DMSO-d₆) δ ppm 7.84 (s, 4H), 7.52 (td, J=8.1, 1.6 Hz, 4H), 7.45-7.36 (m, 6H), 3.61-3.47 (m, 3H), 3.35 (dd, J=14.2.7.5 Hz, 1H), 1.52 (dq, J=12.6.6.4 Hz, 1H), 1.32-1.19 (m, 1H), 0.98 (d, J=7.1 Hz, H), 0.92 (s, 9H), 0.84 (s, 2H), 0.49 (dt, J=8.8, 4.6 Hz, 1H), 0.30 (dt, J=9.6, 5.0 Hz, 1H). [M+H]⁺=484.5.

### Step 7: (1S,2R)-2-(24(tert-butldiphenylsilyl)oxy)ethyl)cyclopropyl)methanamine

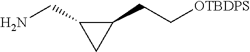

To the solution of 2-(((1S,2R)-2-(2-((tert-butyldiphenylsilyl)oxy)ethyl)cyclopropyl)methyl)isoindoline-1,3-dione (3.5 g, 7.2 mmol) in EtOH (100 mL) was added hydrazine hydrate (6 mL). The resulting mixture was stirred at 70° C. for 2 hours. After LCMS showed the reaction was completed, the mixture was filtered to remove the solids. The filtrate was concentrated under reduced pressure to give the product (2.4 g, 93.7%). [M+H]⁺=354.5.

### Step 8: 5-bromo-N—(((1S,2R)-2-(24(tert-butyldiphenylsilyl)oxy)ethyl)cyclopropyl)methyl)-2-nitroaniline

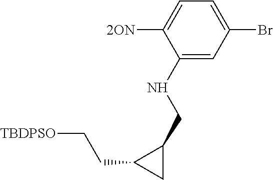

To a solution of ((1S,2R)-2-(2-((tert-butyldiphenylsilyl)oxy)ethyl)cyclopropyl)methanamine (1.0 g, 2.8 mmol) and 4-bromo-2-fluoro-1-nitrobenzene (684 mg, 3.1 mmol) in MeCN (30 mL) was added DIEA (728 mg, 5.6 mol) at 25° C. The mixture was stirred at 60° C. for 12 hours. The reaction concentrated under reduced pressure to give a residue, which was purified by column chromatography (PE:EA=10:1) to give product (1.4 g, 89.7% yield). [M+H]⁺=553.5

### Step 9: 2-((1R,2S)-2-(((5-bromo-2-nitrophenyl)amino)methyl)cyclopropyl)ethan-1-ol

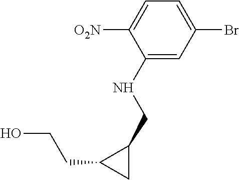 [SMILES: O=[N+]([O-])c1ccc(Br)cc1NC[C@H]1C[C@@H]1CCO]

To the solution of 5-bromo-N—(((1S,2R)-2-(2-((tert-butyldiphenylsilyl)oxy)ethyl)cyclopropyl)methyl)-2-nitroaniline (1.4 g, 2.5 mmol) in THF (10 mL) was added TBAF (1.0 mol/L in THF, 4.0 mL). The resulting mixture was stirred at RT for 3 hours. After LCMS showed the reaction was completed, the reaction was concentrated under reduced pressure to give the crude residue, which was purified by silica column chromatography (PE:EA=2:1) to afford the product (670 mg, 84.1%). [M+H]⁺=315.5.

### Step 10: methyl 2-(5-(2-((1R,2S)-2-(((5-bromo-2-nitrophenyl)amino)methyl)cyclopropyl)ethoxy)-1-methyl-1H-pyrazol-4-yl)-6-methylisonicotinate

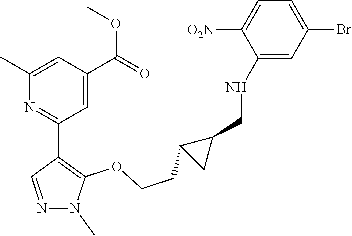 [SMILES: COC(=O)c1cc(C)nc(-c2cnn(C)c2OCC[C@H]2C[C@@H]2CNc2cc(Br)ccc2[N+](=O)[O-])c1]

To a solution of 2-((1R,2S)-2-(((5-bromo-2-nitrophenyl)amino)methyl)cyclopropyl)ethan-1-ol (665 mg, 2.1 mmol), methyl 2-(5-hydroxy-1-methyl-1H-pyrazol-4-yl)-6-methylisonicotinate (576 mg, 2.3 mmol) and PPh₃ (719 mg, 2.7 mmol) in THF (30 mL) was added DIAD (554 mg, 2.7 mmol) dropwise at 15° C. The mixture was stirred at RT for 30 minutes. The reaction mixture was concentrated under reduced pressure to give the crude residue, which was purified by column chromatography (DCM:EA=3:1) to give product (1.0 g, 86.9% yield). [M+H]⁺=544.5.

### Step 11: methyl 2-(5-(2-((1R,2S)-2-(((2-amino-5-bromophenyl)amino)methyl)cyclopropyl)ethoxy)-1-methyl-1H-pyrazol-4-yl)-6-methylisonicotinate

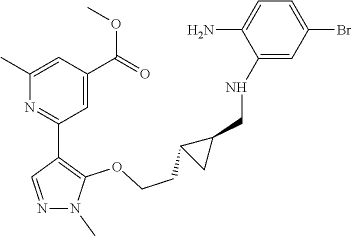 [SMILES: COC(=O)c1cc(C)nc(-c2cnn(C)c2OCC[C@H]2C[C@@H]2CNc2cc(Br)ccc2N)c1]

The title compound was prepared in a procedure similar to that in Example 9 step 4. [M+H]⁺=514.5.

### Step 12: methyl 2-(5-(2-((1R,2S)-2-((6-bromo-2-imino-2,3-dihydro-1H-benzo[d]imidazol-1-yl)methyl)cyclopropyl)ethoxy)-1-methyl-1H-pyrazol-4-yl)-6-methylisonicotinate

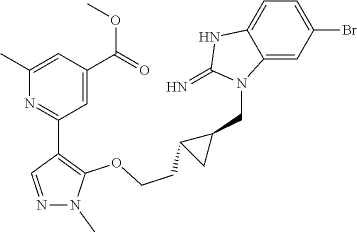 [SMILES: COC(=O)c1cc(C)nc(-c2cnn(C)c2OCC[C@H]2C[C@@H]2Cn2c(=N)[nH]c3ccc(Br)cc32)c1]

The title compound was prepared in a procedure similar to that in Example 9 step 5. [M+H]⁺=539.5.

### Step 13: (7¹S,7²R,E)-5⁶-bromo-1¹,2⁶-dimethyl-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphan-3-one

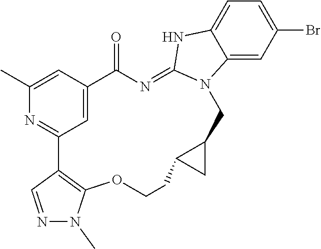 [SMILES: Cc1cc2cc(n1)-c1cnn(C)c1OCC[C@H]1C[C@@H]1CN1/C(=N/C2=O)Nc2ccc(Br)cc21]

To a solution of methyl 2-(5-(2-((1R,2S)-2-((6-bromo-2-imino-2,3-dihydro-1H-benzo[d]imidazol-1-yl)methyl)cyclopropyl)ethoxy)-1-methyl-1H-pyrazol-4-yl)-6-methylisonicotinate (900 mg, 1.7 mmol) in DMF (30 mL) was added LiHMDS (1.0 mol/L in THF, 4.0 mL) dropwise at 0° C. The mixture was stirred at RT for 10 minutes, the mixture was quenched with NH₄Cl/water and extracted by DCM. The organic layer was dried over anhydrous Na₂SO₄, filtered and concentrated under reduced pressure to give the crude residue, which was purified by silica column chromatography (DCM:MeOH=20:1) to afford the product (420 mg, 49.6%). [M+H]⁺=507.5.

### Step 14: tert-butyl 4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazine-1-carboxylate

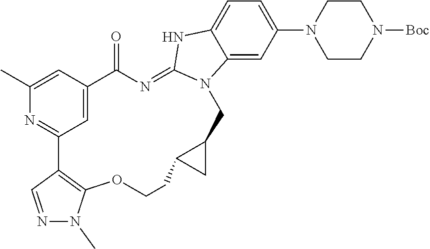 [SMILES: Cc1cc2cc(n1)-c1cnn(C)c1OCC[C@H]1C[C@@H]1CN1/C(=N/C2=O)Nc2ccc(N3CCN(C(=O)OC(C)(C)C)CC3)cc21]

The title compound was prepared in a procedure similar to that in Example 324 step 1. [M+H]⁺=613.5.

### Step 15: (7¹S,7²R,E)-1¹,2⁶-dimethyl-5⁶-(piperazin-1-yl)-5²,5³-dihydro-1¹H,5¹H-10-oxa-4-aza-5(2,1)-benzo[d]imidazola-2(2,4)-pyridina-1(4,5)-pyrazola-7(1.2)-cyclopropanacyclodecaphan-3-one

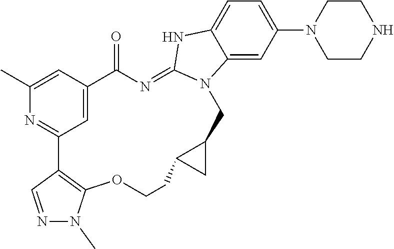 [SMILES: Cc1cc2cc(n1)-c1cnn(C)c1OCC[C@H]1C[C@@H]1CN1/C(=N/C2=O)Nc2ccc(N3CCNCC3)cc21]

The title compound was prepared in a procedure similar to that in Example 324 step 2. [M+H]⁺=513.5.

### Step 16: (R)-3-(4-(4-(4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-2,6-difluorophenyl)piperidine-2,6-dione

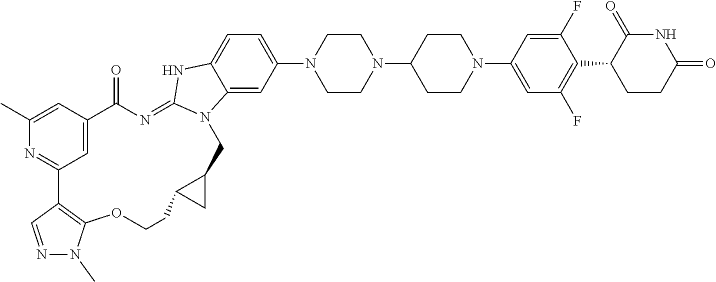 [SMILES: Cc1cc2cc(n1)-c1cnn(C)c1OCC[C@H]1C[C@@H]1CN1/C(=N/C2=O)Nc2ccc(N3CCN(C4CCN(c5cc(F)c([C@H]6CCC(=O)NC6=O)c(F)c5)CC4)CC3)cc21]

The title compound was prepared in a procedure similar to that in Example 324 step 3. ¹H NMR (5(0) MHz, DMSO) δ 12.43 (s, 1H), 10.87 (s, 1H), 8.71 (s, 1H), 7.88 (s, 1H), 7.50 (s, 1H), 7.34 (d, J=8.7 Hz, 1H), 7.04 (s, 1H), 6.89 (d, J=8.6 Hz, 1H), 6.64 (d, J=12.8 Hz, 2H), 4.33-4.23 (m, 3H), 4.12-4.03 (m, 2H), 3.82 (d, J=11.9 Hz, 2H), 3.75 (s, 3H), 3.18-3.12 (m, 4H), 2.81-2.74 (m, 3H), 2.68 (s, 4H), 2.55 (s, 3H), 2.45 (s, 2H), 2.13-2.05 (m, 2H), 1.99-1.94 (m, 1H), 1.88 (d, =11.8 Hz, 2H), 1.53-1.40 (m, 3H), 1.33 (d, J=4.1 Hz, 1H), 1.17-1.11 (m, 1H), 0.53 (s, 1H), 0.41-0.33 (m, 1H). [M+H]⁺=819.6.

### Example 392: (R)-3-(2,6-difluoro-4-((1-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)azetidin-3-yl)amino)phenyl)piperidine-2,6-dione

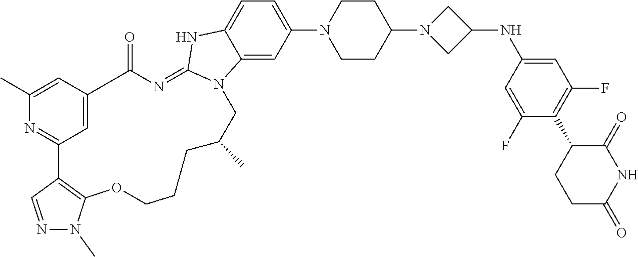 [SMILES: Cc1cc2cc(n1)-c1cnn(C)c1OCCC[C@@H](C)CN1/C(=N/C2=O)Nc2ccc(N3CCC(N4CC(Nc5cc(F)c([C@H]6CCC(=O)NC6=O)c(F)c5)C4)CC3)cc21]

The titled compound was prepared in a procedure similar to that in Example 381. ¹H NMR (500 MHz, DMSO) δ 12.50 (s, 1H), 10.84 (s, 1H), 8.42 (s, 1H), 7.92 (s, 1H), 7.55 (s, 1H), 7.36 (d, J=8.0 Hz, 1H), 7.14(s, 1H), 6.89 (d, J=8.1 Hz, 1H), 6.67 (s, 1H), 6.19 (d, J=12.2 Hz, 2H), 4.36 (s, 1H), 4.13 (d, J=12.5 Hz. IH), 4.02-3.90 (m, 4H), 3.73 (s, 3H), 3.65 (s, 2H), 3.54 (s, 2H), 2.85-2.73 (m, 6H), 2.55 (s, 3H), 2.19 (s, 2H), 2.10-1.85 (m, 5H), 1.76 (s, 2H), 1.48-1.27 (m, 3H), 0.81 (d, J=5.5 Hz, 3H). [M+H]⁺=807.6.

### Example 393: 3-(6-methyl-5-(2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)pyridin-2-yl)piperidine-2,6-dione

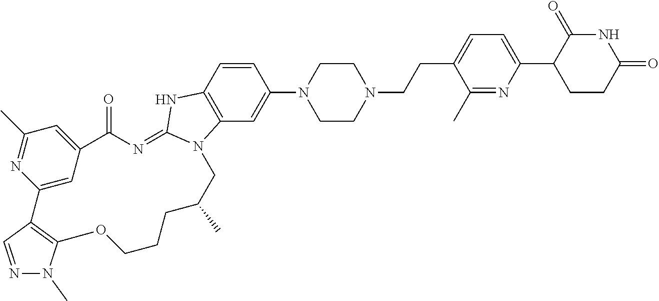 [SMILES: Cc1cc2cc(n1)-c1cnn(C)c1OCCC[C@@H](C)CN1/C(=N/C2=O)Nc2ccc(N3CCN(CCc4ccc(C5CCC(=O)NC5=O)nc4C)CC3)cc21]

The titled compound was prepared in a manner similar to that in Example 373.

¹H NMR (500 MHz, DMSO) δ 12.56 (s, 1H), 10.82 (s, 1H), 8.43 (s, 1H), 7.92 (s, 1H), 7.66-7.51 (m, 2H), 7.41 (s, 1H), 7.23-7.19 (m, 2H), 6.95 (s, 1H), 4.37-4.32 (m, 1H), 4.16-4.13 (m, 1H), 4.04-3.83 (m, 5H), 3.73-3.68 (m, 4H), 3.38-3.35 (m, 1H), 3.18-3.14 (m, 5H), 2.82-2.79 (m, 3H), 2.64-2.61 (m, 6H), 2.23-2.20 (m, 3H), 2.09-2.06 (m, 2H), 2.04-1.87 (m, 2H), 1.46-1.43 (m, 1H), 0.82 (d, J=6.3 Hz, 3H); [M+H]⁺=745.6.

### Example 394: (R)-3-(2,6-difluoro-4-(4-(methyl(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl) amino)piperidin-1-yl)phenyl)piperidine-2,6-dione

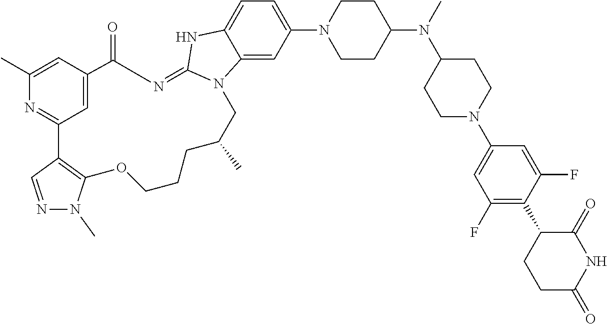 [SMILES: Cc1cc2cc(n1)-c1cnn(C)c1OCCC[C@@H](C)CN1/C(=N/C2=O)Nc2ccc(N3CCC(N(C)C4CCN(c5cc(F)c([C@H]6CCC(=O)NC6=O)c(F)c5)CC4)CC3)cc21]

The titled compound was prepared in a manner similar to that in Example 324.

¹H NMR (500 MHz, DMSO) δ 12.50 (s, 1H), 10.87 (s, 1H), 8.42 (s, 1H), 7.92 (s, 1H), 7.55 (s, 1H), 7.36 (d, J=8.0 Hz, 1H), 7.14 (s, 1H), 6.89 (s, 1H), 6.63 (d, J=13.1 Hz, 2H), 4.39-4.34 (m, 1H), 4.17-4.12 (m, 1H), 4.05-4.01 (m, 1H), 3.99-3.94 (m, 2H), 3.79-3.77 (m, 2H), 3.73 (s, 4H), 2.78-2.71 (m, 6H), 2.21-2.18 (m, 4H), 2.09-2.05 (m, 1H), 1.99-1.94 (m, 2H), 1.79-1.76 (m, 3H), 1.68-1.64 (m, 2H), 1.53-1.49 (m, 3H), 1.23 (s, 8H), 0.87-0.84 (m, 1H), 0.82 (d, J=6.6 Hz, 3H); [M+H]⁺=849.5.

### Example 395: 3-(5-(4-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-yl-)pyridin-2-yl)piperidine-2,6-dione

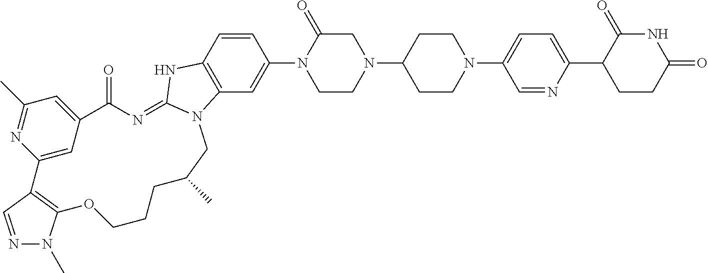 [SMILES: Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5)CC4)CC3=O)cc21]

The titled compound was prepared in a manner similar to that in Example 373.

¹H NMR (500 MHz, DMSO) δ 12.76 (s, 1H), 10.78 (s, 1H), 8.43 (s, 1H), 8.23 (d, J=2.8 Hz, 1H), 7.92 (s, 1H), 7.66 (s, 1H), 7.57 (s, 1H), 7.52 (d, J=8.5 Hz, 1H), 7.35 (dd, J=8.7, 2.9 Hz, 1H), 7.22-7.13 (m, 2H), 4.36 (d, J=4.9 Hz, 1H), 4.18 (d, J=10.9 Hz, 1H), 4.00 (s, 1H), 3.97-3.91 (m, 1H), 3.89 (dd, J=8.8, 5.3 Hz, 1H), 3.79 (d, J=12.2 Hz, 2H), 3.73 (s, 3H), 3.71 (d, J=5.6 Hz, 1H), 3.68-3.60 (m, 1H), 3.34 (s, 2H), 2.92 (t, J=5.1 Hz, 2H), 2.81 (s, 1H), 2.76 (t, J=11.4 Hz, 2H), 2.62-2.52 (m, 5H), 2.26-2.15 (m, 2H), 2.10 (dd, J=12.9, 6.4 Hz, 1H), 1.94 (t, J=15.6 Hz, 4H), 1.63-1.51 (m, 2H), 1.43 (s, 1H), 0.82 (d, J=6.5 Hz, 3H). [M+H]⁺=800.7.

### Example 396: (S)-3-(1-oxo-5-(4-(4-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)isoindolin-2-yl)piperidine-2,6-dione

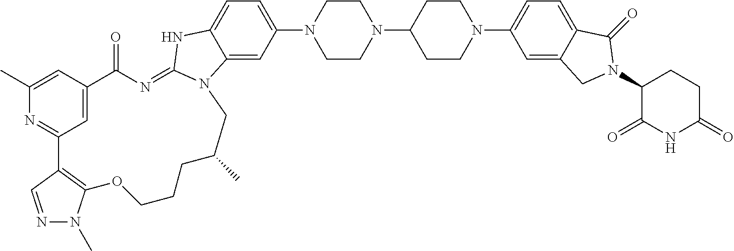 [SMILES: Cc1cc2cc(n1)-c1cnn(C)c1OCCC[C@@H](C)CN1/C(=N/C2=O)Nc2ccc(N3CCN(C4CCN(c5ccc6c(c5)CN([C@H]5CCC(=O)NC5=O)C6=O)CC4)CC3)cc21]

The titled compound was prepared in a manner similar to that in Example 324.

¹H NMR (500 MHz, DMSO) δ 12.49 (s, 1H), 10.95 (s, 1H), 8.42 (s, 1H), 7.92 (s, 1H), 7.55 (s, 1H), 7.51 (d, J=8.4 Hz, 1H), 7.37 (d, 1=8.6 Hz, 1H), 7.14 (s, 1H), 7.07 (d, J=9.1 Hz, 2H), 6.89 (d, J=9.3 Hz, 1H), 5.05 (dd, J=13.1, 4.7 Hz, 1H), 4.41-4.29 (m, 2H), 4.25-4.18 (m, 1H), 4.18-4.11 (m, 1H), 4.03-3.89 (m, 4H), 3.73 (d, J=28.1 Hz, 3H), 3.16 (s, 3H), 2.95-2.82 (m, 4H), 2.69 (s, 4H), 2.55 (s, 3H), 2.37 (d, J=18.4 Hz, 2H), 2.21 (d, J=32.9 Hz, 1H), 2.01-1.89 (m, 4H), 1.61-1.50 (m, 2H), 1.47 (d, J=13.1 Hz, 2H), 1.17 (d, J=30.8 Hz, 2H), 0.82 (d, J=6.4 Hz, 3H). [M+H]=840.4

### Example 397: (R)-3-(2,6-difluoro-4-(4-((S)-2-(methoxymethyl)-4-((R,E)-1¹,2⁶,7-trimethyl-3-oxo-5²,5³-dihydro-1¹H,5¹H-1i-oxa-4-aza-5(2,1)-benzo[d]imidazola-2(2,4)-pyridina-1(4,5)-pyrazolacycloundecaphane-5-yl)piperazin-1-yl)piperidin-1-yl)phenyl)piperidine-2,6-dione

### Step 1: tert-butyl (S)-2-(methoxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylate

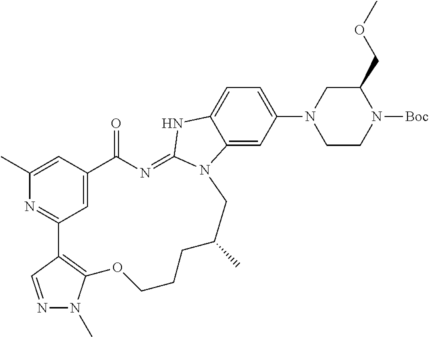 [SMILES: COC[C@@H]1CN(c2ccc3c(c2)N2C[C@H](C)CCCOc4c(cnn4C)-c4cc(cc(C)n4)C(=O)/N=C/2N3)CCN1C(=O)OC(C)(C)C]

To a mixture of (R,E)-5⁶-bromo-1¹,2⁶,7-trimethyl-5²,5-dihydro-1¹H,5¹H-11-oxa-4-aza-5(2,1)-benzo[d]imidazola-2(2,4)-pyridina-1(4,5)-pyrazolacycloundecaphan-3-one (0.5 g, 0.98 mmol), tert-butyl (S)-2-(methoxymethyl)piperazine-1-carboxylate (393 mg, 1.96 mmol), Pd₂(dba)₃ (89 mg, 0.1 mmol), Ruphos (91 mg, 0.2 mmol), NaO^{t}Bu (283 mg, 2.94 mmol) in DMA (10 mL) was stirred at 100° C. for 4 hrs. After cooling to r.t, the reaction was quenched with sat. NH₄Cl solution (15 mL), the resulting mixture was extracted with DCM (20 mL×3). The combined organic phase was washed with brine (20 mL), dried over Na₂SO₄, filtered and concentrated in vacuum. The residue was purified by silica gel column (DCM:CH₃OH=20:1) to afford tert-butyl (S)-2-(methoxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylate (380 mg, 61.6%). [M+H]⁺=659.4.

### Step 2: (R,E)-5⁶-((S)-3-(methoxymethyl)piperazin-1-yl-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

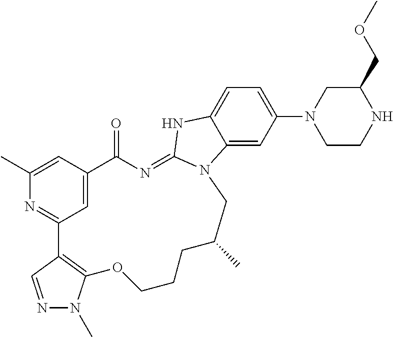

To a solution of tert-butyl (S)-2-(methoxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylat e (0.38 g, 0.57 mmol) in DCM (10 mL) was added TFA (5 mL). The reaction was stirred at r.t for 2 hrs and then concentrated in vacuo. The residue was dissolved in DCM (30 mL), washed with sat. NaHCO₃ solution (20 mL) and brine (20 mL), dried over Na₂SO₄, filtered and concentrated in vacuum to afford (R,E)-5⁶-((S)-3-(methoxymethyl)piperazin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (220 mg, 68.3%). [M+H]⁺=559.5.

### Step 3: (R)-3-(2,6-difluoro-4-(4-((S)-2-(methoxymethyl)-4 (R,E)-1¹,2⁶,7-trimethyl-3-oxo-5²,5³-dihydro-1¹H, 5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

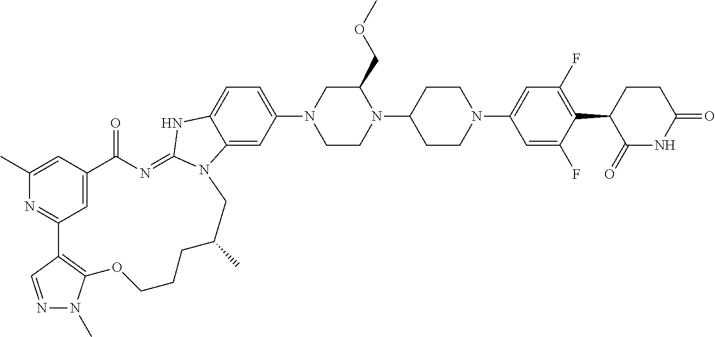 [SMILES: COC[C@@H]1CN(c2ccc3c(c2)N2C[C@H](C)CCCOc4c(cnn4C)-c4cc(cc(C)n4)C(=O)/N=C/2N3)CCN1C1CCN(c2cc(F)c([C@H]3CCC(=O)NC3=O)c(F)c2)CC1]

To a solution of (R,E)-56-((S)-3-(methoxymethyl)piperazin-1-yl)-11,26,7-trimethyl-52,53-dihydro-1¹H,5¹H-11-oxa-4-aza-5(2,1)-benzo[d]imidazola-2(2,4)-pyridina-1(4,5)-pyrazolacycloundecaphan-3-one (120 mg, 0.21 mmol), (R)-3-(2,6-difluoro-4-(4-oxopiperidin-1-yl)phenyl)piperidine-2,6-dione (103 mg, 0.32 mmol) in DCE (10 mL) was added STAB (136 mg, 0.64 mmol). Then the mixture was stirred at 50° C. for 1 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=20:1) to afford (R)-3-(2,6-difluoro-4-(4-((S)-2-(methoxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione (34.3 mg, 18.5%).

¹H NMR (500 MHz, DMSO) δ 12.51 (s, 1H), 10.87 (s, 1H), 8.42 (s, 1H), 7.92 (s, 1H), 7.56 (s, 1H), 7.37 (d, J=8.5 Hz, 1H), 7.10 (s, 1H), 6.86 (d, J=8.0 Hz, 1H), 6.64 (d, J=13.0 Hz, 2H), 4.37 (s, 1H), 4.21-4.12 (m, 1H), 4.07-3.93 (m, 4H), 3.83 (d, J=10.4 Hz, 2H), 3.73 (s, 3H), 3.58-3.43 (m, 2H), 3.43 (s, 1H), 3.30 (s, 3H), 3.20 (s, 1H), 3.03 (s, 2H), 2.94 (s, 1H), 2.81-2.73 (m, 4H), 2.55 (s, 3H), 2.28-2.06 (m, 6H), 2.08-1.86 (m, 5H), 1.75-1.57 (m, 2H), 1.47-1.36 (m, 1H), 0.82 (d, J=6.4 Hz, 2H); [M+H]⁺=865.8.

### Example 398: (R)-3-(4-(4-((2R,5R)-2,5-dimethyl-4-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)-2,6-difluorophenyl)piperidine-2,6-dione

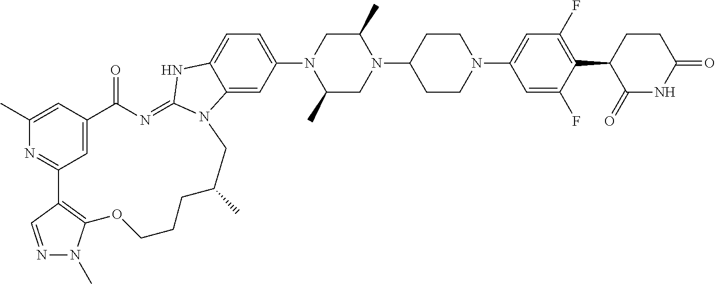 [SMILES: Cc1cc2cc(n1)-c1cnn(C)c1OCCC[C@@H](C)CN1/C(=N/C2=O)Nc2ccc(N3C[C@@H](C)N(C4CCN(c5cc(F)c([C@H]6CCC(=O)NC6=O)c(F)c5)CC4)C[C@H]3C)cc21]

The titled compound was prepared in a manner similar to that in Example 324.

¹H NMR (500 MHz, DMSO) δ 10.87 (s, 1H), 8.42 (s, 1H), 7.92 (d, J=4.1 Hz, 1H), 7.55 (s, 1H), 7.35 (d, J=8.9 Hz, 1H), 7.03 (s, 1H), 6.82 (d, J=7.9 Hz, 1H), 6.64 (d, J=12.8 Hz, 2H), 4.44-4.33 (m, 1H), 4.03-3.93 (m, 4H), 3.87-3.82 (m, 2H), 3.73 (s, 3H), 3.07-3.00 (m, 1H), 2.82-2.74 (m, 6H), 2.59-2.55 (m, 4H), 2.36 (d, J=1.9 Hz, 1H), 2.25-1.84 (m, 8H), 1.76-1.69 (m, 2H), 1.57-1.36 (m, 5H), 1.18 (d, J=6.0 Hz, 2H), 0.97 (d, J=6.2 Hz, 2H), 0.84-0.76 (m, 4H); [M+H]=849.7.

### Example 400: (R)-3-(2,6-difluoro-4-((3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)propyl) amino)phenyl)piperidine-2,6-dione

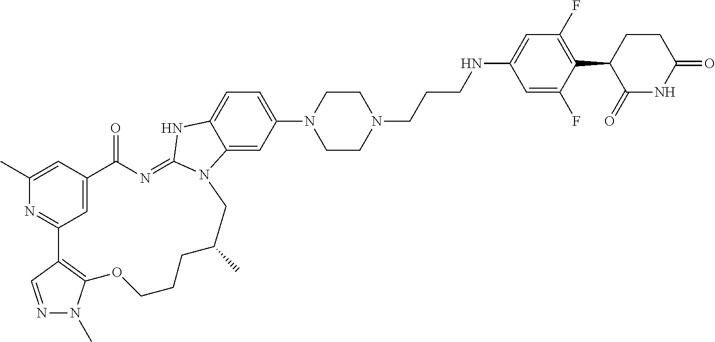 [SMILES: Cc1cc2cc(n1)-c1cnn(C)c1OCCC[C@@H](C)CN1/C(=N/C2=O)Nc2ccc(N3CCN(CCCNc4cc(F)c([C@H]5CCC(=O)NC5=O)c(F)c4)CC3)cc21]

The titled compound was prepared in a manner similar to that in Example 368.

¹H NMR (500 MHz, DMSO) δ 12.51 (s, 1H), 10.83 (s, 1H), 8.42 (s, 1H), 7.92 (s, 1H), 7.56 (s, 1H), 7.37 (d, J=8.7 Hz, 1H), 7.15 (s, 1H), 6.89 (d, J=8.5 Hz, 1H), 6.28 (t, J=4.7 Hz, 1H), 6.24 (d, J=12.1 Hz, 2H), 4.36 (m, 1H), 4.14 (d, J=11.9 Hz, 1H), 4.05-3.93 (m, 3H), 3.73 (s, 3H), 3.19 (s, 4H), 3.07 (d, J=5.8 Hz, 2H), 2.88-2.72 (m, 2H), 2.55 (s, 7H), 2.47 (s, 1H), 2.44 (s, 2H), 2.21 (m, 1H), 2.06 (m, 1H), 2.01 (s, 3H), 1.79-1.67 (m, 2H), 1.40 (s, 1H), 0.82 (d, J=6.3 Hz, 3H). [M+H]⁺=795.4

### Example 401: (R)-3-(2,6-difluoro-4-(4-((1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)amino) piperidin-1-yl)phenyl)piperidine-2,6-dione

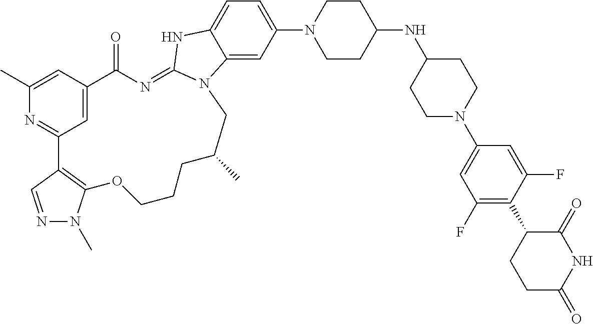 [SMILES: Cc1cc2cc(n1)-c1cnn(C)c1OCCC[C@@H](C)CN1/C(=N/C2=O)Nc2ccc(N3CCC(NC4CCN(c5cc(F)c([C@H]6CCC(=O)NC6=O)c(F)c5)CC4)CC3)cc21]

The titled compound was prepared in a manner similar to that in Example 324.

¹H NMR (500 MHz, DMSO) δ 10.87 (s, 1H), 8.42 (s, 1H), 8.24 (s, 1H), 7.92 (s, 1H), 7.55 (s, 1H), 7.36 (d, J=8.6 Hz, 1H), 7.14 (s, 1H), 6.89 (d, J=8.0 Hz, 1H), 6.63 (d, J=12.9 Hz, 2H), 4.37 (s, 1H), 4.18-4.12 (m, 1H), 4.07-3.95 (m, 4H), 3.78-3.71 (m, 6H), 3.70-3.60 (m, 3H), 2.84-2.75 (m, 8H), 2.55 (s, 3H), 2.22 (s, 1H), 2.14-2.05 (m, 1H), 1.95-1.86 (m, 6H), 1.48-1.41 (m, 3H), 1.35-1.26 (m, 2H), 0.82 (d, J=6.3 Hz, 3H); [M+H]⁺=835.6.

### Example 402: (R)-3-(2,6-difluoro-4-((1-(1-((R,E)-1¹,2⁶,7-trimethyl-3-oxo-5²,53-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)piperidin-4-yl)amino)phenyl)piperidine-2,6-dione

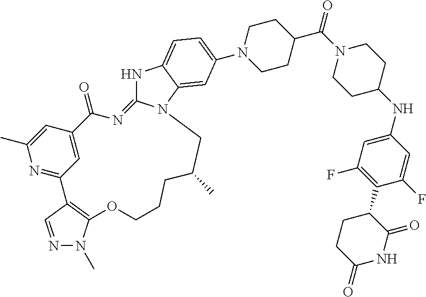 [SMILES: Cc1cc2cc(n1)-c1cnn(C)c1OCCC[C@@H](C)CN1/C(=N/C2=O)Nc2ccc(N3CCC(C(=O)N4CCC(Nc5cc(F)c([C@H]6CCC(=O)NC6=O)c(F)c5)CC4)CC3)cc21]

The title compound was prepared in a procedure similar to that in Example 333. ¹H NMR (500 MHz, DMSO) δ_{H} 12.51 (s, 1H), 10.83 (s, 1H), 8.42 (s, 1H), 7.92 (s, 1H), 7.56 (s, 1H), 7.37 (d, J=8.7 Hz, 1H), 7.15 (s, 1H), 6.91 (d, J=8.4 Hz, 1H), 6.29 (d, J=12.2 Hz, 2H), 6.19 (d, J=7.9 Hz, 1H), 4.36 (s, 1H), 4.26 (d, J=11.5 Hz, 1H), 4.14 (d, J=11.6 Hz, 1H), 4.02-3.95 (m, 4H), 3.76-3.70 (m, 5H), 3.51 (s, 1H), 3.07 (s, 2H), 2.84-2.76 (m, 6H), 2.55 (s, 3H), 2.23 (s, 2H), 2.10-1.85 (m, 5H), 1.73 (s, 4H), 1.45 (s, 1H), 1.31-1.18 (m, 2H), 0.82 (d, J=6.2 Hz, 3H). [M+H]⁺=863.6.

### Example 403: (R)-3-(2,6-difluoro-4-((1-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-4-carbonyl)azetidin-3-yl)oxy)phenyl)piperidine-2,6-dione

### Step 1: tert-butyl 3-(4-(2,6-bis(benzyloxy)pyridin-3-yl)-3,5-difluorophenoxy)azetidine-1-carboxylate

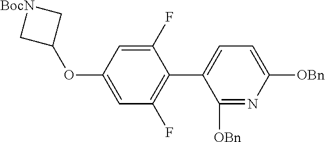 [SMILES: CC(C)(C)OC(=O)N1CC(Oc2cc(F)c(-c3ccc(OCc4ccccc4)nc3OCc3ccccc3)c(F)c2)C1]

To a solution of 4-(2,6-bis(benzyloxy)pyridin-3-yl)-3,5-difluorophenol (21 g, 50.11 mmol) in DMF (210 mL) was added tert-butyl 3-(methylsulfonyl)oxy)azetidine-1-carboxylate (18.87 g, 75.18 mmol) and Cs₂CO₃(48.87 g, 150.37 mmol). The resulting solution was stirred for 2h at 110° C. under N₂ atmosphere. After cooling to rt, the resulting solution was diluted with H₂O and extracted with EtOAc. The combined organic layer was washed with brine, dried by Na₂SO₄ and concentrated. The filtrate was concentrated under reduced pressure. The crude product purified by silica column chromatography (DCM:PE=0-40%) to afford tert-butyl 3-(4-(2,6-bis(benzyloxy)pyridin-3-yl)-3,5-difluorophenoxy)azetidine-1-carboxylate (15 g, 52.16%). [M+H]⁺=575.6

### Step 2: tert-butyl 3-(4-(2,6-dioxopiperidin-3-yl)-3,5-difluorophenoxy)azetidine-1-carboxylate

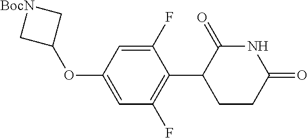 [SMILES: CC(C)(C)OC(=O)N1CC(Oc2cc(F)c(C3CCC(=O)NC3=O)c(F)c2)C1]

A round bottom flask equipped with a magnetic stirrer, was charged with tert-butyl 3-(4-(2,6-bis(benzyloxy)pyridin-3-yl)-3,5-difluorophenoxy)azetidine-1-carboxylate (15 g, 26.13 mmol), dry THF (150 ml), and Pd/C (10 wt %, 15 g). The resulting mixture was degassed under reduced pressure and purged with H₂ for five times, then stirred overnight at 50° C. The mixture was diluted with THF/EA (50 mL/50 mL), then sonicated for 10 minutes, followed by filtration through a pad of celite. The filtrate was concentrated under vacuum, The residue was purified by silica column chromatography (EA:PE=0-40%) to afford tert-butyl 3-(4-(2,6-dioxopiperidin-3-yl)-3,5-difluorophenoxy)azetidine-1-carboxylate (7 g, 17.68 mmol, 67.63%). [M+H]⁺=397.4.

### Step 3: tert-butyl (R)-3-(4-(2,6-dioxopiperidin-3-yl)-3,5-difluorophenoxy)azetidine-1-carboxylate

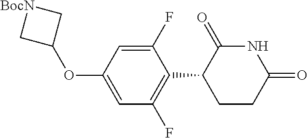 [SMILES: CC(C)(C)OC(=O)N1CC(Oc2cc(F)c([C@H]3CCC(=O)NC3=O)c(F)c2)C1]

The tert-butyl 3-(4-(2,6-dioxopiperidin-3-yl)-3,5-difluorophenoxy)azetidine-1-carboxylate (15 g) was separated by prer-chiral-sfc with following condition: (Column: CHIRALPAK IG-3, 4.6*50 mm 3 um; Flow rate: 4 mL/min; Gradient: 10% to 50% in 2.0 min, hold 1.0 min at 50%, Injection Volume: 5 ul mL) to afford tert-butyl (R)-3-(4-(2,6-dioxopiperidin-3-yl)-3,5-difluorophenoxy)azetidine-1-carboxylate (3 g, 85.7%, ee=99.94%). H NMR (400 MHz, DMSO-d₆) δ 10.95 (s, 1H), 6.74-6.65 (m, 2H), 5.02 (m, 1H), 4.38-4.28 (m, 1H), 4.16 (m, 1H), 3.84-3.76 (m, 2H), 3.34 (s, 1H), 2.81 (m, 2H), 2.19-1.93 (m, 2H), 1.39 (s, 9H). [M+H]⁺=397.4.

### Step 4: tert-butyl (S)-3-(4-(2,6-dioxopiperidin-3-yl)-3,5-difluorophenoxy)azetidine-1-carboxylate

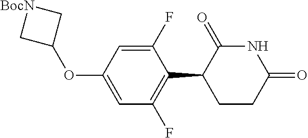 [SMILES: CC(C)(C)OC(=O)N1CC(Oc2cc(F)c([C@@H]3CCC(=O)NC3=O)c(F)c2)C1]

The tert-butyl 3-(4-(2,6-dioxopiperidin-3-yl)-3,5-difluorophenoxy)azetidine-I-carboxylate (15 g) was separated by prer-chiral-sfc with following condition: (Column: CHIRALPAK IG-3, 4.6*50 mm 3 um: Flow rate: 4 mL/min; Gradient: 10% to 50% in 2.0 min, hold 1.0 min at 50%, Injection Volume: 5 ul mL) to afford tert-butyl (S)-3-(4-(2,6-dioxopiperidin-3-yl)-3,5-difluorophenoxy)azetidine-1-carboxylate (3 g, 85.7%, ee=99.88%). ¹H NMR (400 MHz, DMSO-d₆) δ 10.95 (s, 1H), 6.74-6.65 (m, 2H), 5.02 (m, 1H), 4.38-4.28 (m, 1H), 4.16 (m, 1H), 3.84-3.76 (m, 2H), 3.34 (s, 1H), 2.81 (m, 2H), 2.19-1.93 (m, 2H), 1.39 (s, 9H). [M+H]⁺=397.4.

### Step 5: (R)-3-(4-(azetidin-3-yloxy)-2,6-difluorophenyl)piperidine-2,6-dione

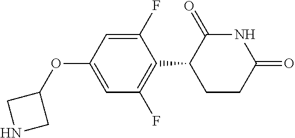 [SMILES: O=C1CC[C@H](c2c(F)cc(OC3CNC3)cc2F)C(=O)N1]

To a 50-mL round-bottomed flask was added tert-butyl (R)-3-(4-(2,6-dioxopiperidin-3-yl)-3,5-difluorophenoxy)azetidine-1-carboxylate (400 mg, 1.0 mmol), DCM (6 mL) and TFA (2 mL). After stirring for 1 h at room temperature, the reaction mixture was concentrated under reduced pressure to afford the product (350 mg), which was used without further purification. [M+H]⁺=297.5.

### Step 6: (R)-3-(2,6-difluoro-4-((1-(1-((R,E)-1¹,2⁶,7-trimethyl-3-oxo-5²,5³-dihydro-1¹H,5¹H-11-oxa-4-aza-5(2,1-benzo[d]imidazola-2(2,4)-pyridina-1(45)-pyrazolacycloundecaphane-5⁶-yl)piperidine-4-carbonyl)azetidin-3-yl)oxy)phenyl)piperidine-2,6-dione

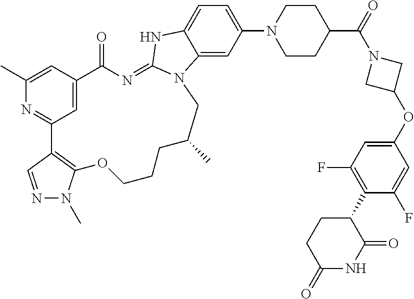 [SMILES: Cc1cc2cc(n1)-c1cnn(C)c1OCCC[C@@H](C)CN1/C(=N/C2=O)Nc2ccc(N3CCC(C(=O)N4CC(Oc5cc(F)c([C@H]6CCC(=O)NC6=O)c(F)c5)C4)CC3)cc21]

The title compound was prepared in a procedure similar to that in Example 5 step 7. ¹H NMR (500 MHz, DMSO) δ_{H} 12.50 (s, 1H), 10.94 (s, 1H), 8.42 (s, 1H), 7.92 (s, 1H), 7.55 (s, 1H), 7.36 (d, J=8.7 Hz, 1H), 7.15 (s, 1H), 6.90 (d, J=8.6 Hz, 1H), 6.73 (d, J=10.4 Hz, 2H), 5.09 (s, 1H), 4.70 (t, J=7.7 Hz, 1H), 4.39-4.31 (m, 2H), 4.22-4.10 (m, 3H), 3.98 (t, J=11.5 Hz, 2H), 3.82 (d, J=9.1 Hz, 1H), 3.75-3.67 (m, 5H), 3.16 (s, 2H), 2.85-2.70 (m, 4H), 2.55 (s, 3H), 2.43 (s, 1H), 2.21-2.10 (m, 2H), 2.02-1.94 (m, 2H), 1.78-1.64 (m, 4H), 1.44 (s, 1H), 0.82 (d, J=6.4 Hz, 3H). [M+H]⁺=836.6.

### Example 404: (R)-3-(2,6-difluoro-4-((1-(1-((R,E)-1¹,2⁶,7-trimethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁴-yl)piperidine-4-carbonyl)azetidin-3-yl)amino)phenyl)piperidine-2,6-dione

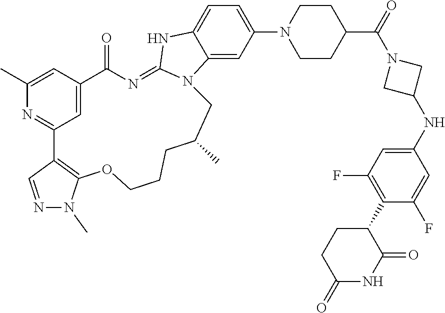 [SMILES: Cc1cc2cc(n1)-c1cnn(C)c1OCCC[C@@H](C)CN1/C(=N/C2=O)Nc2ccc(N3CCC(C(=O)N4CC(Nc5cc(F)c([C@H]6CCC(=O)NC6=O)c(F)c5)C4)CC3)cc21]

The title compound was prepared in a procedure similar to that in Example 333. ¹H NMR (500 MHz, DMSO) δ 12.50 (s, 1H), 10.86 (s, 1H), 8.42 (s, 1H), 7.92 (d, J=1.7 Hz, 1H), 7.56 (s, 1H), 7.37 (d, J=7.2 Hz, 1H), 7.15 (s, 1H), 6.95-6.86 (m, 2H), 6.22 (d, J=12.6 Hz, 2H), 4.59 (s, 1H), 4.36 (s, 1H), 4.22 (s, 2H), 4.14 (d, J=13.8 Hz, 1H), 4.05-3.90 (m, 5H), 3.75-3.65 (m, 5H), 2.83-2.70 (m, 5H), 2.55 (s, 3H), 2.42 (s, 1H), 2.21 (s, 1H), 2.10-1.90 (m, 4H), 1.77-1.65 (m, 4H), 1.45 (s, 1H), 0.81 (s, 3H). [M+H]⁺=835.6.

### Example 405: (S)-3-(5-(methyl(2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)amino)-1-oxoisoindolin-2-yl)piperidine-2,6-dione

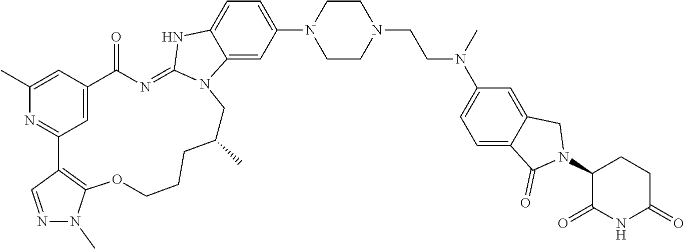 [SMILES: Cc1cc2cc(n1)-c1cnn(C)c1OCCC[C@@H](C)CN1/C(=N/C2=O)Nc2ccc(N3CCN(CCN(C)c4ccc5c(c4)CN([C@H]4CCC(=O)NC4=O)C5=O)CC3)cc21]

The titled compound was prepared in a manner similar to that in Example 260. ¹H NMR (500 MHz, DMSO) δ 12.50 (s, 1H), 10.93 (s, 1H), 8.42 (s, 1H), 7.92 (s, 1H), 7.56 (s, 1H), 7.50 (d, J=9.5 Hz, 1H), 7.37 (d, J=9.0 Hz, 1H), 7.15 (s, 1H), 6.89 (d, J=9.0 Hz, 1H), 6.83 (d, J=9.0 Hz, 1H), 6.82 (s, 1H), 5.03 (dd, J=13.5, 5.0 Hz, 1H), 4.39-4.30 (m, 2H), 4.21-4.13 (m, 2H), 4.00-3.95 (m, 2H), 3.73 (s, 3H), 3.63-3.60 (m, 2H), 3.38-3.33 (m, 4H), 3.21-3.13 (m, 4H), 3.04 (s, 3H), 2.94-2.79 (m, 2H), 2.67-2.57 (m, 6H), 2.41-2.31 (m, 1H), 2.26-2.17 (m, 1H), 2.01-1.88 (m, 3H), 1.49-1.40 (m, 1H), 0.81 (d, J=6.5 Hz, 3H). [M+H]⁺=814.7

### Example 406: (R)-3-(2,6-difluoro-4-(3-((3-oxo-2-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-2,8-diazaspiro[4.5]decan-8-yl)methyl)azetidin-1-yl)phenyl)piperidine-2,6-dione

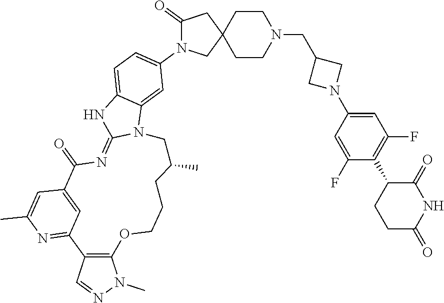 [SMILES: Cc1cc2cc(n1)-c1cnn(C)c1OCCC[C@@H](C)CN1/C(=N/C2=O)Nc2ccc(N3CC4(CCN(CC5CN(c6cc(F)c([C@H]7CCC(=O)NC7=O)c(F)c6)C5)CC4)CC3=O)cc21]

The titled compound was prepared in a manner similar to that in Example 374.

¹H NMR (500 MHz, DMSO) δ 12.80 (s, 1H), 10.86 (d, J=12.5 Hz, 1H), 8.52 (s, 1H), 8.05 (s, 1H), 7.86 (s, 1H), 7.70 (s, 1H), 7.53 (t, J=9.4 Hz, 2H), 6.15 (d, J=11.2 Hz, 2H), 4.38 (d, J=4.1 Hz, 2H), 4.21 (d, J=11.0 Hz, 2H), 4.08-4.03 (m, 4H), 3.90 (t, J=15.2 Hz, 2H), 3.75 (s, 3H), 3.64 (s, 2H), 3.46 (s, 2H), 3.28-3.01 (m, 4H), 2.88-2.75 (m, 2H), 2.68 (s, 1H), 2.63 (s, 3H), 2.56-2.51 (m, 2H), 2.15-1.76 (m, 9H), 1.47 (s, 1H), 0.86-0.80 (m, 3H). [M+H]⁺=875.7.

### Example 407: (R)-3-(2,6-difluoro-4-(methyl(2-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl) ethyl)amino)phenyl)piperidine-2,6-dione

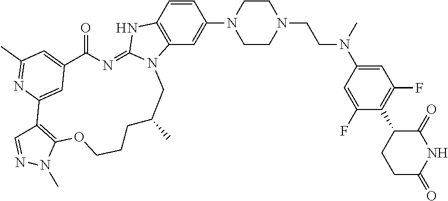 [SMILES: Cc1cc2cc(n1)-c1cnn(C)c1OCCC[C@@H](C)CN1/C(=N/C2=O)Nc2ccc(N3CCN(CCN(C)c4cc(F)c([C@H]5CCC(=O)NC5=O)c(F)c4)CC3)cc21]

The titled compound was prepared in a manner similar to that in Example 368.

¹H NMR (500 MHz, DMSO) δ 12.51 (s, 1H), 10.85 (s, 1H), 8.42 (s, 1H), 7.92 (s, 1H), 7.55 (s, 1H), 7.37 (d, J=8.7 Hz, 1H), 7.15 (s, 1H), 6.89 (d, J=8.1 Hz, 1H), 6.38 (d, J=13.3 Hz, 2H), 4.37 (s, 1H), 4.14 (d, J=12.3 Hz, 1H), 4.00 (t, J=11.9 Hz, 3H), 3.73 (s, 3H), 3.50 (s, 2H), 3.31-3.28 (m, 1H), 3.20-3.12 (m, 4H), 2.94 (s, 3H), 2.83-2.73 (m, 2H), 2.73-2.58 (m, 4H), 2.62-2.51 (m, 5H), 2.22 (s, 1H), 2.08 (d, J=12.7 Hz, 1H), 1.96 (s, 3H), 1.45 (s, 1H), 0.81 (d, J=6.3 Hz, 3H). [M+H]⁺=795.7

### Example 418: (R)-3-(2,6-difluoro-4-(4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)-4,7-diazaspiro[2.5]octan-7-yl)piperidin-1-yl)phenyl)piperidine-2,6-dione

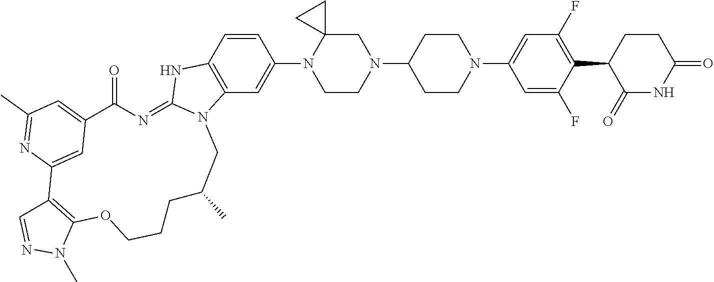 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC34CC4)cc21]

The titled compound was prepared in a manner similar to that in Example 421.

¹H NMR (500 MHz, DMSO) δ 12.46 (s, 1H), 10.86 (s, 1H), 8.42 (s, 1H), 7.92 (s, 1H), 7.55 (s, 1H), 7.34 (d, J=8.8 Hz, 1H), 7.14 (s, 1H), 6.94 (d, J=8.1 Hz, 1H), 6.60 (d, J=12.8 Hz, 2H), 4.40-4.34 (m, 1H), 4.17 (d, J=11.6 Hz, 1H), 4.06-3.97 (m, 2H), 3.95-3.87 (m, 1H), 3.75 (s, 1H), 3.73 (s, 4H), 2.81-2.70 (m, 4H), 2.55 (s, 4H), 2.54-2.51 (m, 6H), 2.43-2.36 (m, 1H), 2.33-2.29 (m, 1H), 2.25-2.17 (m, 1H), 2.12-2.03 (m, 1H), 2.01-1.86 (m, 3H), 1.75 (d, J=12.2 Hz, 2H), 1.49-1.37 (m, 3H), 0.82 (d, J=6.4 Hz, 4H), 0.68 (s, 2H); [M+H]⁺=847.7.

### Example 421: (R)-3-(2,6-difluoro-4-(4-((R)-2-(hydroxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

### Step 1: (R,E-5⁶-((R)-3-(hydroxymethyl)piperazin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

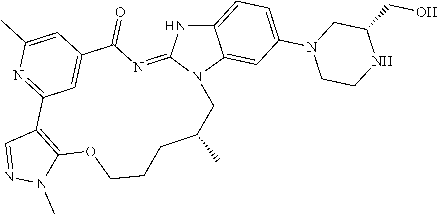 [SMILES: Cc1cc2cc(n1)-c1cnn(C)c1OCCC[C@@H](C)CN1/C(=N/C2=O)Nc2ccc(N3CCN[C@@H](CO)C3)cc21]

A mixture of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (508 mg, 1 mmol), tert-butyl (R)-2-(hydroxymethyl)piperazine-1-carboxylate (325 mg, 1.5 mmol), Pd₂dba₃(91 mg, 0.1 mmol), Ruphos (93 mg, 0.2 mmol), and ^{t}-BuONa (288 mg, 3 mmol) in DMA (10 mL) was stirred in a round bottom flask at 90° C. for 2 hours under N2. Water (20 mL) was added, and the mixture was extracted with DCM (50 mL×3). The combined organic layer was dried over Na₂SO₄. The solvent was removed by evaporation, and the residue was purified by silica gel column chromatography to afford the product (350 mg, 64%). [M+H]⁺=545.2.

### Step 2: (R)-3-(2,6-difluoro-4444(R)-2(hydroxymethyl)-4-((R,E)-1¹,2⁶,7-trimethyl-3-oxo-5²,5³-dihydro-1H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

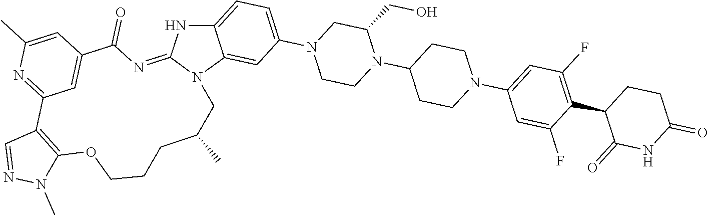 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@@H](CO)C3)cc21]

A mixture of (R,E)-5⁶-((R)-3-(hydroxymethyl)piperazin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (200 mg, 0.36 mmol) and (R)-3-(2,6-difluoro-4-(4-oxopiperidin-1-yl)phenyl)piperidine-2,6-dione (130 mg, 0.39 mmol) in 1,2-dichloromethane (15 mL)) was stirred in a round bottom flask at 70° C. for 2 hours. To the mixture was added NaBH(OAc)₃ (155 mg, 0.72 mmol) and the reaction was stirred in a round bottom flask at 70° C. for 12 hours. Then the mixture was evaporated in vacuum to afford the crude product, which was purified with silica gel column chromatography (DCM:MeOH=100:0˜80:20 gradient elution) to give the product (15 mg, 5%). ¹H NMR (500 MHz, DMSO) δ 12.48 (s, 1H), 10.87 (s, 1H), 8.42 (s, 1H), 7.92 (s, 1H), 7.55 (s, 1H), 7.37 (d, J=8.7 Hz, 1H), 7.08 (s, 1H), 6.87 (d, J=8.5 Hz, 1H), 6.61-6.59 (m, 2H), 4.63 (s, 1H), 4.36-4.32 (m, 1H), 4.15-4.12 (m, 1H), 4.05-4.02 (m, 1H), 4.02-3.90 (m, 2H), 3.83-3.81 (m, 2H), 3.73-3.69 (m, 3H), 3.66-3.62 (m, 1H), 3.57-3.54 (m, 1H), 3.29-3.22 (m, 3H), 2.98-2.96 (m, 3H), 2.78-2.75 (m, 6H), 2.64-2.61 (m, 1H), 2.55-2.51 (m, 3H), 2.29-2.14 (m, 1H), 2.14-2.03 (m, 1H), 2.02-1.85 (m, 4H), 1.78-1.68 (m, 1H), 1.68-1.57 (m, 1H), 1.51-1.39 (m, 2H), 0.82 (d, J=6.4 Hz, 3H), [M+H]⁺=851.6.

### Example 422: (R)-3-(2,6-difluoro-4-(3-(((R)-2-(hydroxymethyl)-4-((R,E)-1¹,2⁶,7-trimethyl-3-oxo-5²,5³-dihydro-1¹H,5¹H-li-oxa-4-aza-5(2,1)-benzo[d]imidazola-2(2,4)-pyridina-1(4,5)-pyrazolacycloundecaphane-5⁶-yl)piperazin-1-yl)methyl)azetidin-1-yl)phenyl)piperidine-2,6-dione

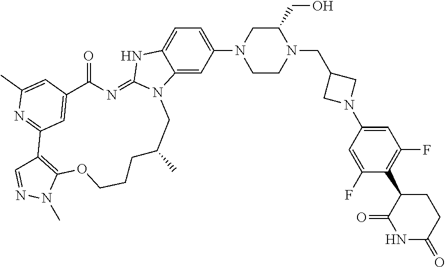 [SMILES: Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)[C@@H](CO)C3)cc21]

The titled compound was prepared in a manner similar to that in Example 421. ¹H NMR (500 MHz, DMSO) δ 12.51 (s, 1H), 10.85 (s, 1H), 8.42 (s, 1H), 7.92 (s, 1H), 7.56 (s, 1H), 7.38 (d, J=8.7 Hz, 1H), 7.12 (s, 1H), 6.89 (d, J=8.3 Hz, 1H), 6.12 (d, J=11.2 Hz, 2H), 4.63 (s, 1H), 4.37 (d, J=4.4 Hz, 1H), 4.15 (d, J=11.5 Hz, 1H), 4.07-3.88 (m, 5H), 3.73 (s, 3H), 3.69-3.65 (m, 1H), 3.49-3.43 (m, 7H), 3.09-3.05 (m, 1H), 3.01-2.85 (m, 3H), 2.86-2.72 (m, 3H), 2.62-2.54 (m, 4H), 2.41-2.39 (m, 1H), 2.27-2.14 (m, 1H), 2.08-2.06 (m, 1H), 2.02-1.89 (m, 3H), 1.52-1.40 (m, 1H), 0.82 (d, J=6.4 Hz, 3H); [M+H]⁺=837.6.

### Example 423: (R)-1-((1-(4-((R)-2,6-dioxopiperidin-3-yl)-3,5-difluorophenyl)azetidin-3-yl)methyl)-4-((R,E)-1¹,2⁶,7-trimethyl-3-oxo-5²,5³-dihydro-1¹H,5¹H-1l-oxa-4-aza-5(2,1)-benzo[d]imidazola-2(2,4)-pyridina-1(4,5)-pyrazolacycloundecaphane-5⁶-yl)piperazine-2-carbonitrile

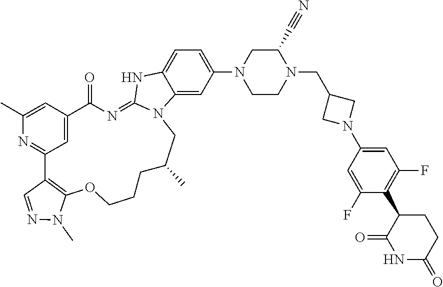 [SMILES: Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)[C@@H](C#N)C3)cc21]

The titled compound was prepared in a manner similar to that in Example 421.

¹H NMR (500 MHz, DMSO) δ 10.86 (s, 1H), 8.43 (s, 1H), 7.92 (s, 1H), 7.56 (s, 1H), 7.41 (d, J=8.7 Hz, 1H), 7.24 (s, 1H), 6.93 (d, J=8.1 Hz, 1H), 6.13 (d, J=11.0 Hz, 2H), 4.35-4.32 (m, 2H), 4.15-4.12 (m, 1H), 4.08-3.92 (m, 6H), 3.84-3.80 (m, 1H), 3.73-3.71 (m, 4H), 3.68-3.59 (m, 1H), 3.55-3.53 (m, 2H), 3.01-2.95 (m, 1H), 2.93-2.91 (m, 2H), 2.78-2.75 (m, 5H), 2.55-2.52 (m, 4H) 2.22-2.19 (m, 1H), 2.08-2.06 (m, 1H), 1.94-1.92 (m, 3H), 1.44-1.42 (m, 1H), 0.82 (d, J=6.3 Hz, 3H): [M+H]⁺=832.6.

### Example 424: (R)-3-(2,6-difluoro-4-((3S,4S)-3-fluoro-4-(4-((R,E)-1¹,2⁶,7-trimethyl-3-oxo-5²,5¹-dihydro-1¹H,5¹H-11-oxa-4-aza-5(2,1)-benzo[d]imidazola-2(2,4)-pyridina-1(4,5)-pyrazolacycloundecaphane-5-yl)piperazin-1-yl)piperidin-1-yl)phenyl)piperidine-2,6-dione

### Step 1: tert-butyl (3S,4S)-4-(4-benzylpiperazin-1-yl)-3-fluoropiperidine-1-carboxylate

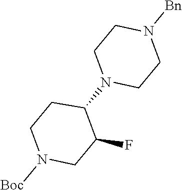 [SMILES: CC(C)(C)OC(=O)N1CC[C@H](N2CCN(Cc3ccccc3)CC2)[C@@H](F)C1]

A mixture of tert-butyl (3S,4S)-4-amino-3-fluoropiperidine-1-carboxylate (1 g, 4.58 mmol), NaHCO₃ (1.54 g, 18.3 mmol) and N-benzyl-2-chloro-N-(2-chloroethyl)ethan-1-amine (1.28 g, 5.5 mmol) in EtOH (10 mL) was stirred in a round bottom flask at 70° C. for 12 hours under N₂. Water (20 mL) was added, and the mixture was extracted with DCM (50 mL×3). The combined organic layer was dried over Na₂SO₄. The solvent was removed by evaporation, and the residue was purified by silica gel column chromatography to afford the product (1.2 g, 69%). [M+H]⁺=378.2.

### Step 2: 1-benzyl-4-((3S,4S)-3-fluoropiperidin-4-yl)piperazine

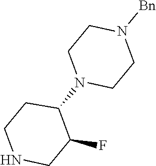 [SMILES: F[C@H]1CNCC[C@@H]1N1CCN(Cc2ccccc2)CC1]

tert-butyl (3S,4S)-4-(4-benzylpiperazin-1-yl)-3-fluoropiperidine-1-carboxylate (1.2 g, 3.18 mmol) in DCM (4 mL) and TFA (1 mL). After stirring for 1 h at room temperature, the reaction mixture was concentrated under reduced pressure. To the residue was added DCM (30 mL) and sat. aq. NaHCO₃ (20 mL), and the layers were separated. The aqueous layer was extracted with DCM (3×30 mL). The combined organic layers were washed with brine (50 mL), dried over anhydrous Na₂SO₄, filtered and concentrated under reduced pressure to afford the product (800 mg, 90%), which was used without further purification. [M+H]⁺=278.2.

### Step 3: 1-benzyl-4-((3S,4S)-1-(4-(2,6-bis(benzyloxy)pyridin-3-yl)-3,5-difluorophenyl)-3-fluoropiperidin-4-yl)piperazine

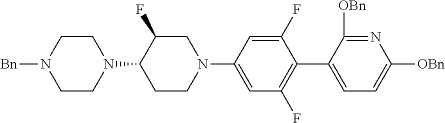 [SMILES: Fc1cc(N2CC[C@H](N3CCN(Cc4ccccc4)CC3)[C@@H](F)C2)cc(F)c1-c1ccc(OCc2ccccc2)nc1OCc1ccccc1]

To a stirred solution of 2,6-bis(benzyloxy)-3-(4-bromo-2,6-difluorophenyl)pyridine (1.13 g, 2.34 mmol) and 1-benzyl-4-((3S,4S)-3-fluoropiperidin-4-yl)piperazine (500 mg, 1.8 mmol) in dioxane (50 mL) were added Cs₂CO₃ (1.17 g, 3.6 mmol), Xantphos (210 mg, 0.36 mmol) and Pd₂(dba)₃ (164 mg, 0.18 mmol) at room temperature under nitrogen atmosphere. The resulting mixture was stirred for 16 h at 100° C. under nitrogen atmosphere. The residue was diluted with EtOAc (100 mL), washed with water (3×100 mL) and brine (100 mL). The organic layer was dried over anhydrous Na₂SO₄, filtered and concentrated under reduced pressure. The residue was purified by silica gel column chromatography, eluted with PE/EtOAc (5:1) to afford the product (300 mg, 25%); [M+H]⁺=679.6.

### Step 4: 3-(2,6-difluoro-4-((3S,4S)-3-fluoro-4-(piperazin-1-yl)piperidin-1-yl)phenyl)piperidine-2,6-dione

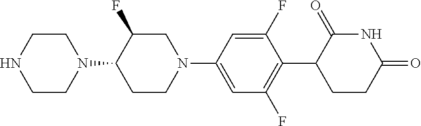 [SMILES: O=C1CCC(c2c(F)cc(N3CC[C@H](N4CCNCC4)[C@@H](F)C3)cc2F)C(=O)N1]

1-benzyl-4-((3S,4S)-1-(4-(2,6-bis(benzyloxy)pyridin-3-yl)-3,5-difluorophenyl)-3-fluoropiperidin-4-yl)piperazine (300 mg, 0.44 mmol) was dissolved in DMF (10 mL) and iPr-OH (10 mL). Pd/C (100 mg, 10 wt. %, wet) was added to the solution in one portion. The resulting mixture was stirred under hydrogen atmosphere (0 atm) at room temperature overnight. The solid was filtered off and the filtrate was concentrated to give the crude product (160 mg, 88%). [M+H]⁺=411.4.

### Step 5: (R)-3-(2,6-difluoro-4-((3S,4S)-3-fluoro-4-(4-((R,E)-1¹,2⁶,7-trimethyl-3-oxo-5²,5³-dihydro-1¹H,5¹H-11-oxa-4-aza-5(2,1)-benzo[d]imidazola-2(2,4-pyridina-1(4,5)-pyrazolacycloundecaphane-5⁶-yl)piperazin-1-yl)piperidin-1-yl)phenyl)piperidine-2,6-dione

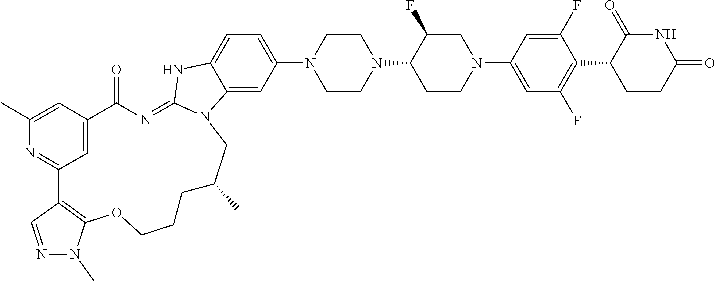 [SMILES: Cc1cc2cc(n1)-c1cnn(C)c1OCCC[C@@H](C)CN1/C(=N/C2=O)Nc2ccc(N3CCN([C@H]4CCN(c5cc(F)c([C@H]6CCC(=O)NC6=O)c(F)c5)C[C@@H]4F)CC3)cc21]

A mixture of 3-(2,6-difluoro-4-((3S,4S)-3-fluoro-4-(piperazin-1-yl)piperidin-1-yl)phenyl)piperidine-2,6-dione (160 mg, 0.39 mmol) and (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (200 mg, 0.39 mmol) Pd₂dba₃(31 mg, 0.039 mmol), Ruphos (32 mg, 0.078 mmol), and BuONa (88 mg, 0.91 mmol) in DMA (10 mL) was stirred in a round bottom flask at 90° C. for 2 hours under N₂. Water (20 mL) was added, and the mixture was extracted with DCM (50 mL×3). The combined organic layer was dried over Na₂SO₄. The solvent was removed by evaporation, and the residue was purified by silica gel column chromatography to afford the product (50 mg, 15%). [M+H]⁺=839.6. The residue was purified by SFC (IG (2*25 cm, Sum), 13% EtOH/87% CO₂, 100 bar, 2 ml/min) and the title compound corresponded to peak A @-, 3.767 min/254 nm (15 mg, 30%).

¹H NMR (500 MHz, DMSO) δ 12.54 (s, 1H), 10.86 (s, 1H), 8.43 (s, 1H), 7.92 (s, 1H), 7.56 (s, 1H), 7.41 (d, J=8.7 Hz, 1H), 7.24 (s, 1H), 6.93 (d, J=8.1 Hz, 1H), 6.13 (d, J=11.0 Hz, 2H), 4.35-4.31 (m, 2H), 4.15-4.12 (m, 1H), 4.09-3.92 (m, 6H), 3.84-3.82 (m, 1H), 3.73-3.68 (m, 3H), 3.63-3.61 (m, 1H), 3.55-3.52 (m, 2H), 3.01-2.96 (m, 1H), 2.93-2.90 (m, 2H), 2.78-2.76 (m, 6H), 2.55-2.53 (m, 4H), 2.22-2.19 (m, 1H), 2.02-1.99 (m, 5H), 1.44-1.41 (m, 1H), 0.82 (d, J=6.3 Hz, 3H); [M+H]⁺=839.6.

### Example 425: (R)-3-(2,6-difluoro-4-(4-(2-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

### Step 1: tert-butyl 3-oxo-4-(piperidin-4-yl)piperazine-1-carboxylate

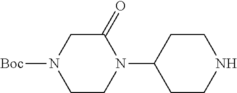 [SMILES: CC(C)(C)OC(=O)N1CCN(C2CCNCC2)C(=O)C1]

The title compound was prepared according to the same procedure as shown in WO 2012176123A1.

### Step 2: tert-butyl 4-(1-(4-(2,6-bis(benzyloxy)pyridin-3-yl)-3,5-difluorophenyl)piperidin-4-yl)-3-oxopiperazine-1-carboxylate

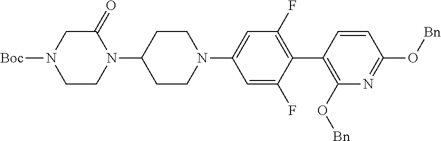 [SMILES: CC(C)(C)OC(=O)N1CCN(C2CCN(c3cc(F)c(-c4ccc(OCc5ccccc5)nc4OCc4ccccc4)c(F)c3)CC2)C(=O)C1]

To a stirred solution of tert-butyl 3-oxo-4-(piperidin-4-yl)piperazine-1-carboxylate (0.7 g, 2.47 mmol) and 2,6-bis(benzyloxy)-3-(4-bromo-2,6-difluorophenyl)pyridine (1.2 g, 2.27 mmol) in dioxane (20 mL) were added Cs₂CO₂ (1.16 g, 4.94 mmol) and Ruphos Pd G3 (0.2 g, 0.25 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (200 mL), washed with water (3×100 mL) and brine (100 mL). The organic layer was dried over anhydrous Na₂SO₄, filtered and concentrated under reduced pressure. The residue was purified by silica gel column chromatography, eluted with PE/EtOAc (4: 1) to afford the product (1.5 g, 88.7%); [M+H]⁺=685.1.

### Step 3: tert-butyl 4-(1-(4-(2,6-dioxopiperidin-3-yl)-3,5-difluorophenyl)piperidin-4-yl)-3-oxopiperazine-1-carboxylate

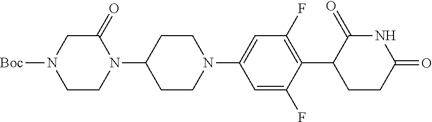 [SMILES: CC(C)(C)OC(=O)N1CCN(C2CCN(c3cc(F)c(C4CCC(=O)NC4=O)c(F)c3)CC2)C(=O)C1]

Tert-butyl 4-(1-(4-(2,6-bis(benzyloxy)pyridin-3-yl)-3,5-difluorophenyl)piperidin-4-yl)-3-oxopiperazine-1-carboxylate (1.5 g, 2.2 mmol) was dissolved in DMF (30 mL) and iPr-OH (30 mL). Pd/C (2.3 g, 10 wt. %, wet) was added to the solution in one portion. The resulting mixture was stirred under hydrogen atmosphere (1 atm) at room temperature overnight. The solid was filtered off and the filtrate was concentrated to give the crude product. The crude was purified by silica gel column chromatography, eluted with DMC/MeOH (20: 1) to afford the product (0.9 g, 81.1%); [M+H]⁺=507.5.

### Step 4: 3-(2,6-difluoro-4-(4-(2-oxopiperazin-1-yl)piperidin-1-yl)phenyl)piperidine-2,6-dione

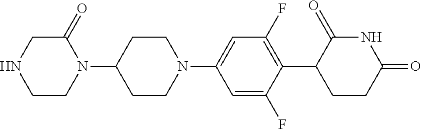 [SMILES: O=C1CCC(c2c(F)cc(N3CCC(N4CCNCC4=O)CC3)cc2F)C(=O)N1]

To a 100-mL round-bottomed flask equipped with a magnetic stir bar was added tert-butyl 4-(1-(4-(2,6-dioxopiperidin-3-yl)-3,5-difluorophenyl)piperidin-4-yl)-3-oxopiperazine-1-carboxylate (900 mg, 1.78 mmol), DCM (10 mL) and TFA (5 mL). After stirring for 1 h at room temperature, the reaction mixture was concentrated under reduced pressure to afford the product as TFA salt (920 mg), which was used without further purification. [M+H]⁺=407.4.

### Step 5: (R)-3-(2,6-difluoro-4-(4-(2-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

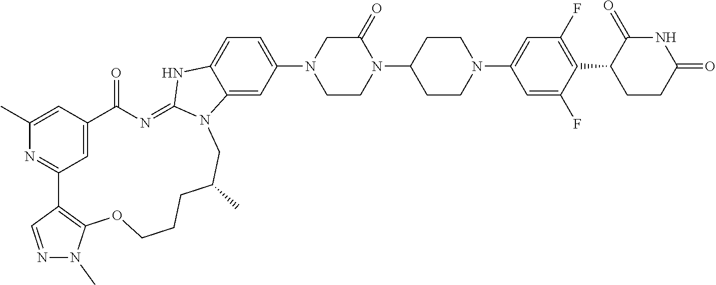 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)C(=O)C3)cc21]

To a stirred solution of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (150 mg, 0.29 mmol) and 3-(2,6-difluoro-4-(4-(2-oxopiperazin-1-yl)piperidin-1-yl)phenyl)piperidine-2,6-dione (107 mg, 0.26 mmol) in DMA (10 mL) were added t-BuONa (141 mg, 1.47 mmol), Ruphos (55 mg, 0.12 mmol) and Pd₂(dba)₃ (54 mg, 0.06 mmol) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (15: 1) to afford the crude product. Chiral separation by HPLC afforded the title compound. HPLC conditions: Column: CHIRALPAK IE-3; Column Size: 2 cm×25 cm, 5 um; Mobile Phase: MtBE (0.1% FA):(MeOH:DCM=1:1)=30:70; Flow Rate: 1.0 mL/min; Retention Time: 3.73. ¹H NMR (500 MHz, DMSO) δ 12.53 (s, 1H), 10.88 (s, 1H), 8.42 (s, 1H), 7.92 (s, 1H), 7.56 (s, 1H), 7.40 (d, J=8.7 Hz, 1H), 7.19 (s, 1H), 6.89 (d, J=8.7 Hz, 1H), 6.68 (d, J=12.8 Hz, 2H), 4.52-4.47 (m, 1H), 4.37-4.34 (m, 1H), 4.15-4.13 (m, 1H), 4.09-3.96 (m, 3H), 3.94-3.81 (m, 4H), 3.73 (s, 3H), 3.48 (s, 2H), 3.40 (s, 2H), 2.87-2.79 (m, 4H), 2.55 (s, 3H), 2.52 (s, 1H), 2.22 (s, 1H), 2.14-2.06 (m, 1H), 2.03-1.89 (m, 3H), 1.80-1.76 (m, 2H), 1.64-1.62 (m, 2H), 1.44 (s, 1H), 0.82 (d, J=6.3 Hz, 3H): [M+H]⁺=835.8.

### Example 437: (R)-3-(2,6-difluoro-4-(4-(2-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)piperidin-1-yl)phenyl)piperidine-2,6-dione

### Step 1: (R)-3-(2,6-difluoro-4-(4-(2-hydroxyethyl)piperidin-1-yl)phenyl)piperidine-2,6-dione

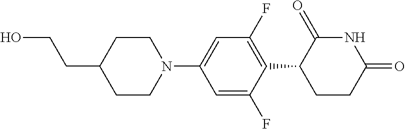 [SMILES: O=C1CC[C@H](c2c(F)cc(N3CCC(CCO)CC3)cc2F)C(=O)N1]

The title compound was obtained through Chiral separation of 3-(2,6-difluoro-4-(4-(2-hydroxyethyl)piperidin-1-yl)phenyl)piperidine-2,6-dione (obtained by the same method in WO2022012622A1) by HPLC. Column: CHIRALPAK IF-3; Column size: 0.46*5 cm; 3 um; Mobile Phase: MTBE (0.1% DEA):(MEOH:DCM=1:1)=50:50; Flow: 1.0 ml/min; The desired enantiomer corresponded to the peak (@; 0.996 min. 4.5 g of desired enantiomer was obtained from 10.0 g of racemate after SFC separation. [M+1]⁺=353.3

### Step 2: (R)-2-(1-(4-(2,6-dioxopiperidin-3-yl)-3,5-difluorophenyl)piperidin-4-yl)acetaldehyde

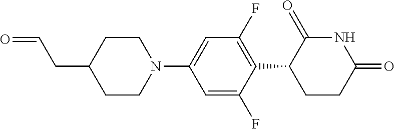 [SMILES: O=CCC1CCN(c2cc(F)c([C@H]3CCC(=O)NC3=O)c(F)c2)CC1]

A mixture of (R)-3-(2,6-difluoro-4-(4-(2-hydroxyethyl)piperidin-1-yl)phenyl)piperidine-2,6-dione (1.0 g, 2.8 mmol) and IBX (1.6 g, 5.7 mmol) in DMSO (40 mL) was stirred in a flask at room temperature overnight. The reaction was quenched with water (200 mL) and the mixture was extracted with EtOAc (60 mL×3). The combined organic layers were washed with sat. aq. NaCl (60 mL×3). sat. aq. NaHCO₃(60 mL×2), dried over anhydrous Na₂SO₄, filtered and concentrated under reduced pressure. The crude product was purified by column chromatography (2%-5% MeOH in DCM) to afford the product (800 mg, 80.4%). [M+H]⁺=351.1.

### Step 3: (R)-3-(2,6-difluoro-4-(4-(2-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)ethyl)piperidin-1-yl)phenyl)piperidine-2,6-dione

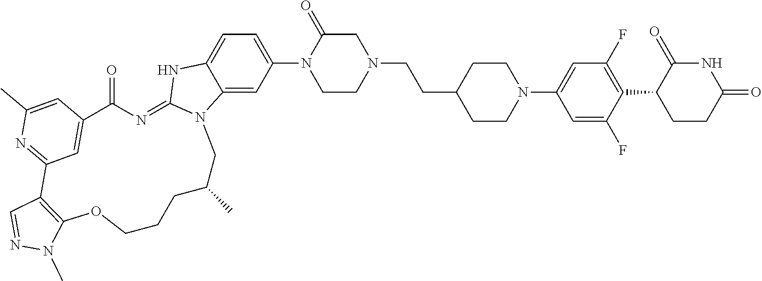 [SMILES: Cc1cc2cc(n1)-c1cnn(C)c1OCCC[C@@H](C)CN1/C(=N/C2=O)Nc2ccc(N3CCN(CCC4CCN(c5cc(F)c([C@H]6CCC(=O)NC6=O)c(F)c5)CC4)CC3=O)cc21]

A solution of (R,E)-1¹,2⁶,7-trimethyl-5⁶-(2-oxopiperazin-1-yl)-5²,5³-dihydro-¹H,5¹H-11-oxa-4-aza-5(2,1)-benzo[d]imidazola-2(2,4)-pyridina-1(4,5)-pyrazolacycloundecaphan-3-one (53.0 mg, 0.10 mmol), (R)-2-(1-(4-(2,6-dioxopiperidin-3-yl)-3,5-difluorophenyl)piperidin-4-yl)acetaldehyde (42.0 mg, 0.12 mmol), and NaBH(OAc)₃ (42.0 mg, 0.20 mmol) in DCM (5 mL) was stirred for 4 h at 30° C., and then the reaction mixture was concentrated under reduced pressure. Purification by flash column chromatography (0-20% MeOH in DCM) to afford the title compound (50.1 mg, 58.0%). 1H NMR (500 MHz, DMSO) δ 12.74 (s, 1H), 10.87 (s, 1H), 8.43 (s, 1H), 7.92 (s, 1H), 7.65 (s, 1H), 7.57 (s, 1H), 7.52 (d, J=8.4 Hz, 1H), 7.20-7.12 (n, 1H), 6.62 (d, J=12.8 Hz, 2H), 4.36 (d, J=4.2 Hz, 1H), 4.18 (d, J=10.8 Hz, 1H), 4.08-3.89 (m, 3H), 3.81-3.61 (m, 7H), 3.18 (s, 2H), 2.81-2.71 (m, 6H), 2.56 (s, 3H), 2.52 (s, 2H), 2.46 (s, 2H), 2.21 (s, 1H), 2.09 (dt, J=13.1, 9.4 Hz, 1H), 1.95 (dd. J=17.7, 12.7 Hz, 3H), 1.76 (d, J=12.3 Hz, 2H), 1.55 (s, 1H), 1.49-1.37 (m, 3H), 1.26-1.15 (m, 2H), 0.82 (d, J=6.5 Hz, 3H). [M+H]⁺=863.7.

### Example 438: (R)-3-(2,6-difluoro-4-(4-(3-(methyl((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidin-1-yl)piperidin-1-yl)phenyl)piperidine-2,6-dione

### Step 1: tert-butyl (R,E)-3-(methyl(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidine-1-carboxylate

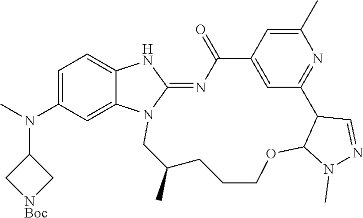 [SMILES: Cc1cc2cc(n1)C1C=NN(C)C1OCCC[C@@H](C)CN1/C(=N/C2=O)Nc2ccc(N(C)C3CN(C(=O)OC(C)(C)C)C3)cc21]

A mixture of (R,E)-5⁶-bromo-1¹,2⁶,7-trimethyl-5²,5³-dihydro-1¹H,5′ H-11-oxa-4-aza-5(2,1)-benzo[d]imidazola-2(2,4)-pyridina-1(4,5)-pyrazolacycloundecaphan-3-one (1 g, 1.97 mmol), tert-butyl 3-(methylamino)azetidine-1-carboxylate (549 mg, 2.95 mmol), Pd₂(dba)₃ (183 mg, 0.2 mmol), Ruphos (187 mg, 0.4 mmol), NaOtBu (568 mg, 5.91 mmol) in DMA (20 mL) was stirred at 100° C. for 15 hrs. After cooling to rt, the reaction was quenched with sat. NH₄Cl solution (15 mL), and the resulting mixture was extracted with DCM (20 mL×3). The combined organic phase was washed with brine (20 mL), dried over Na₂SO₄, filtered and concentrated in vacuum. The residue was purified by silica gel column (DCM:CH₃OH=20:1) to afford: tert-butyl (R,E)-3-(methyl(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidine-1-carboxylate (1.1 g, 90.9%). [M+H]⁺=615.5.

### Step 2: (R,E)-5⁶-(azetidin-3-yl(methyl)amino)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

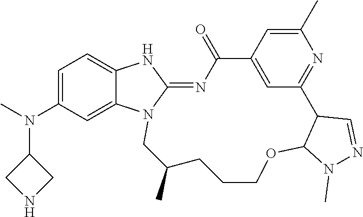 [SMILES: Cc1cc2cc(n1)C1C=NN(C)C1OCCC[C@@H](C)CN1/C(=N/C2=O)Nc2ccc(N(C)C3CNC3)cc21]

To a solution of tert-butyl (R,E)-3-(methyl(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidine-1-carboxylate (1 g, 1.63 mmol) in DCM (15 mL) was added TFA (5 mL). The reaction was stirred at rt for 2 hrs and then concentrated in vacuo. The residue was dissolved in DCM (30 mL), washed with sat. NaHCO₃ solution (20 mL) and brine (20 mL), dried over Na₂SO₄, filtered and concentrated in vacuum to afford (R,E)-5⁶-(azetidin-3-yl(methyl)amino)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (750 mg, 89.4%). [M+H]⁺=515.5.

### Step 3: (R)-3-(2,6-difluoro-4-(4-(3-(methyl((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidin-1-yl)piperidin-1-yl)phenyl)piperidine-2,6-dione

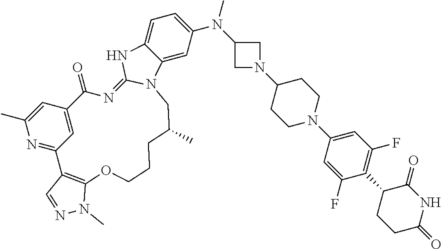 [SMILES: Cc1cc2cc(n1)-c1cnn(C)c1OCCC[C@@H](C)CN1/C(=N/C2=O)Nc2ccc(N(C)C3CN(C4CCN(c5cc(F)c([C@H]6CCC(=O)NC6=O)c(F)c5)CC4)C3)cc21]

To a solution of: (R,E)-5⁶-(azetidin-3-yl(methyl)amino)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 0.19 mmol), (R)-3-(2,6-difluoro-4-(4-oxopiperidin-1-yl)phenyl)piperidine-2,6-dione (81.3 mg, 0.25 mmol) in DCE (6 mL). Then the mixture was stirred at 50° C. for 1 hr. the resulting. The residue was dissolved in DCM (30 mL), washed with sat. NaHCO₃ solution (20 mL) and brine (20 mL), dried over Na₂SO₄ and concentrated in vacuum. The residue was purified by silica gel column (DCM:CH₃OH=94:6) to afford (R)-3-(2,6-difluoro-4-(4-(3-(methyl((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidin-1-yl)piperidin-1-yl)phenyl)piperidine-2,6-dione (31 mg, 19.9%) ¹H NMR (500 MHz, DMSO) δ 12.48 (s, 1H), 10.87 (s, 1H), 8.42 (s, 1H), 7.92 (s, 1H), 7.55 (s, 1H), 7.36 (d, J=8.7 Hz, 1H), 6.92 (d, J=1.8 Hz, 1H), 6.73 (dd, J=8.8, 2.0 Hz, 1H), 6.63 (s, 1H), 6.60 (s, 1H), 4.36 (td, J=9.0, 4.5 Hz, 1H), 4.14 (d, J=13.1 Hz, 1H), 4.09-3.92 (m, 4H), 3.73 (s, 3H), 3.69 (t, J=6.0 Hz, 2H), 3.60 (dd, J=8.4, 4.3 Hz, 2H), 2.95-2.84 (m, 4H), 2.82 (s, 3H), 2.80-2.73 (m, 2H), 2.55 (s, 3H), 2.52 (s, 1H), 2.22 (dd, J=20.7, 16.5 Hz, 2H), 2.15-2.03 (m, 1H), 2.02-1.86 (m, 3H), 1.77-1.64 (M, 2H), 1.55-1.36 (m, 1H), 1.32-1.11 (m, 2H), 0.81 (d, J=6.5 Hz, 3H). [M+H]⁺=821.6.

### Example 439: (R,E)-N—((S)-1-(4-((R)-2,6-dioxopiperidin-3-yl)-3,5-difluorophenethyl)-3,3-dimethylpiperidin-4-yl)-N,1¹,2⁶,7-tetramethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-carboxamide

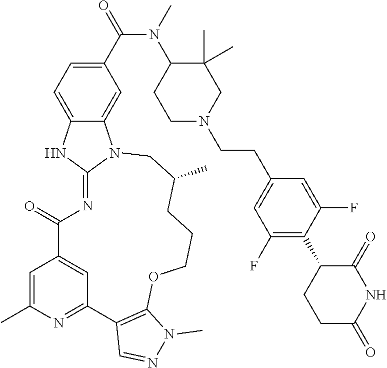 [SMILES: Cc1cc2cc(n1)-c1cnn(C)c1OCCC[C@@H](C)CN1/C(=N/C2=O)Nc2ccc(C(=O)N(C)C3CCN(CCc4cc(F)c([C@H]5CCC(=O)NC5=O)c(F)c4)CC3(C)C)cc21]

The titled compound was prepared in a manner similar to that in Example 260.

¹H NMR (500 MHz, DMSO) δ 12.85 (s, 1H), 10.93 (d, J=15.5 Hz, 1H), 8.43 (s, 1H), 7.93 (d, J=2.9 Hz, 1H), 7.62 (d, J=22.9 Hz, 1H), 7.57 (s, 2H), 7.23-7.16 (m, 1H), 7.04-6.94 (m, 2H), 4.44-4.28 (m, 1H), 4.26-4.11 (m, 2H), 4.10-3.94 (m, 2H), 3.73 (s, 3H), 2.98 (s, 1H), 2.89-2.63 (m, 8H), 2.57 (d, J=11.8 Hz, 5H), 2.48-2.45 (m, 3H), 2.28-1.64 (m, 8H), 1.56-1.35 (m, 3H), 1.24-1.13 (m, 3H), 0.82 (d, J=5.7 Hz, 4H). [M+H]⁺=850.7.

### Example 440: (R)-3-(5-((3S,4S)-3-fluoro-4-(4-((R,L)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)-6-methylpyridin-2-yl)piperidine-2,6-dione

### Step 1: 2′,6′-bis(benzyloxy)-5-bromo-6-methyl-2,3′-bipyridine

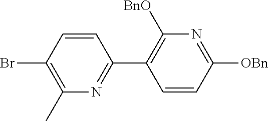 [SMILES: Cc1nc(-c2ccc(OCc3ccccc3)nc2OCc2ccccc2)ccc1Br]

To a solution of 3-bromo-6-iodo-2-methylpyridine (2 g, 6.71 mmol) in dioxane:H₂O (5:1) (30 mL) were added 2,6-bis(benzyloxy)-3-(4,4,5,5-tetramethyl-1,3,2-dioxaborolan-2-yl)pyridine (2.8 g, 6.71 mmol), Pd(dppf)Cl₂ (0.49 g, 0.67 mmol), K₂CO₃ (2.8 g, 20.1 mmol), The resulting solution was stirred for 8 h at 90° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (10:1) to afford 2′,6′-bis(benzyloxy)-5-bromo-6-methyl-2,3′-bipyridine (2.5 g, 80.7%) as a yellow oil. [M+H]⁺=461.2.

### Step 2: 2′,6′-bis(benzyloxy)-5-((3S,4S)-4-(4-benzylpiperazin-1-yl)-3-fluoropiperidin-1-yl)-6-methyl-2,3′-bipyridine

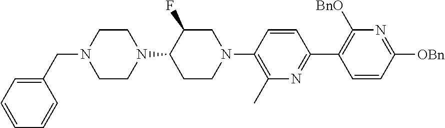 [SMILES: Cc1nc(-c2ccc(OCc3ccccc3)nc2OCc2ccccc2)ccc1N1CC[C@H](N2CCN(Cc3ccccc3)CC2)[C@@H](F)C1]

To a solution of 1-benzyl-4-((3S,4S)-3-fluoropiperidin-4-yl)piperazine (700 mg, 2.52 mmol) in 1,4-dioxane (10 mL) were added 2′,6′-bis(benzyloxy)-5-bromo-6-methyl-2,3′-bipyridine (1.16 g, 2.52 mmol), Pd₂(dba)₃ (230.53 mg, 0.25 mmol), Ruphos (233.4 mg, 0.50 mmol), Cs₂CO₃ (1.65 g, 5.04 mmol), The resulting solution was stirred for 2 h at 100° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (10:1) to afford 2′,6′-bis(benzyloxy)-5-((3S,4S)-444-benzylpiperazin-1-yl)-3-fluoropiperidin-1-yl)-6-methyl-2,3′-bipyridine (700 mg, 42.1%). [M+H]⁺=658.3

### Step 3: 3(5-((3S,4S)-3-fluoro-4-(piperazin-1-yl)piperidin-1-yl)-6-methylpyridin-2-ylpiperidine-2,6-dione

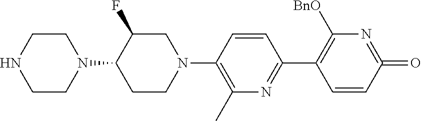

To a solution of 2′,6′-bis(benzyloxy)-5-((3S,4S)-4-(4-benzylpiperazin-1-yl)-3-fluoropiperidin-1-yl)-6-methyl-2,3′-bipyridine (700 mg, 1.06 mmol) in dry DMF:iPrOH (1:1) (30 ml), was added Pd/C (10 wt %, 70 mg). The resulting mixture was degassed under reduced pressure and purged with H₂ (g) for five times, then stirred overnight at 40° C. The mixture was diluted with DMF, then sonicated in an ultrasonic washer for 10 minutes, followed by filtration through a pad of celite. The filtrate was concentrated under vacuum, The residue was purified by silica column chromatography (EA:PE=0-40%) to afford 3-(5-((3S,4S)-3-fluoro-4-(piperazin-1-yl)piperidin-1-yl)-6-methylpyridin-2-yl)piperidine-2,6-dione (40 mg, 9.65%). [M+H]⁺=390.5.

### Step 4: (R)-3-(5-((3S,4S)-3-fluoro-4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)-6-methylpyridin-2-yl)piperidine-2,6-dione

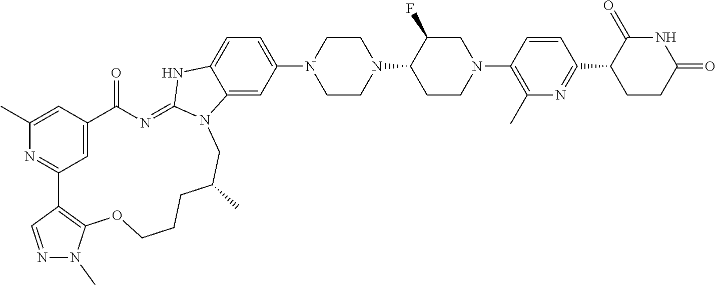 [SMILES: Cc1cc2cc(n1)-c1cnn(C)c1OCCC[C@@H](C)CN1/C(=N/C2=O)Nc2ccc(N3CCN([C@H]4CCN(c5ccc([C@H]6CCC(=O)NC6=O)nc5C)C[C@@H]4F)CC3)cc21]

To a solution of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (40 mg, 0.078 mmol) in 1,4-dioxane (4 mL) were added 3-(5-((3S,4S)-3-fluoro-4-(piperazin-1-yl)piperidin-1-yl)-6-methylpyridin-2-yl)piperidine-2,6-dione (40 mg, 0.11 mmol), Pd₂(dba)₃(7.1 mg, 0.0078 mmol), Ruphos (3.7 mg, 0.0016 mmol), Cs₂CO₃ (77.3 mg, 0.23 mmol). The resulting solution was stirred for 2 h at 100° C. under N2(g) atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with DCM/MeOH (10:1) to afford (R)-3-(5-((3S,4S)-3-fluoro-4-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)-6-methylpyridin-2-yl)piperidine-2,6-dione (10 mg, 15.7%). ¹H NMR (500 MHz, DMSO-d₆): δ 12.52 (s, 1H), 10.79 (s, 1H), 8.43 (s, 1H), 7.92 (s, 1H), 7.56 (s, 1¹H), 7.43 (d, J=8.2 Hz, 1H), 7.38 (d, J=8.7 Hz, 1H), 7.16 (s, 1H), 7.13 (d, J=8.1 Hz, 1H), 6.91 (d, J=10.3 Hz, 1H), 4.96-4.76 (m, 1H), 4.36 (tt, J=14.8, 7.5 Hz, 1H), 4.15 (d, J=13.7 Hz, 1H), 4.05-3.95 (m, 2H), 3.91 (dd, J=9.5, 5.3 Hz, 1H), 3.73 (s, 3H), 3.22-3.14 (m, 4H), 3.08 (d, J=9.6 Hz, 1H), 2.83-2.88 (m, 4H), 2.80-2.74 (m, 3H), 2.69-2.60 (m, 2H), 2.52-2.55 (m, 5H), 2.41 (s, 3H), 2.28-2.15 (m, 2H), 2.13-2.04 (m, 1H), 2.02-1.83 (m, 3H), 1.74 (d, J=16.0 Hz, 1H), 1.50-1.38 (m, 1H), 0.82 (d, J=6.5 Hz, 3H). [M+H]⁺=818.9

### Example 441: (R)-3-(4-(4-(4-((7′R,7²S,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-2,6-difluorophenyl)piperidine-2,6-dione

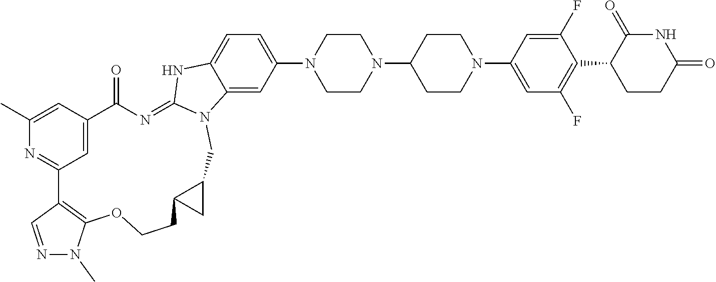 [SMILES: Cc1cc2cc(n1)-c1cnn(C)c1OCC[C@@H]1C[C@H]1CN1/C(=N/C2=O)Nc2ccc(N3CCN(C4CCN(c5cc(F)c([C@H]6CCC(=O)NC6=O)c(F)c5)CC4)CC3)cc21]

The titled compound was prepared in a manner similar to that in Example 421.

¹H NMR (500 MHz, DMSO) δ 12.44 (s, 1H), 10.87 (s, 1H), 8.71 (s, 1H), 7.88 (s, 1H), 7.50 (s, 1H), 7.35 (d, J=9.0 Hz, 1H), 7.05 (s, 1H), 6.89 (d, J=9.5 Hz, 1H), 6.65 (d, J=12.8 Hz, 2H), 4.34-4.23 (m, 3H), 4.11 (s, 1H), 4.08-4.02 (m, 1H), 3.85-3.79 (m, 2H), 3.62 (d, J=9.2 Hz, 1H), 3.29 (s, 2H), 3.16 (s, 4H), 2.77 (t, J=11.2 Hz, 3H), 2.69 (s, 4H), 2.55 (s, 3H), 2.52 (s, 2H), 2.09 (d, J=11.1 Hz, 2H), 2.00-1.93 (m, 1H), 1.88 (d, J=12.8 Hz, 2H), 1.49 (d, J=12.4 Hz, 3H), 1.34 (s, 1H), 1.15 (s, 1H), 0.53 (d, J=4.0 Hz, 1H), 0.40-0.34 (m, 1H). [M+H]⁺=819.6.

### Example 442: 2-((R)-1-(2-((4-((R)-2,6-dioxopiperidin-3-yl)-3,5-difluorophenyl)amino)ethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-2-yl)acetonitrile

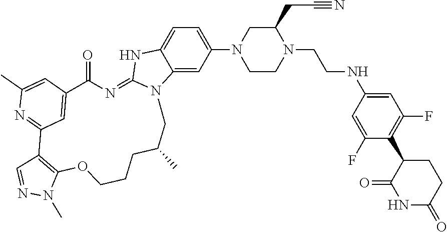 [SMILES: Cc1cc2cc(n1)-c1cnn(C)c1OCCC[C@@H](C)CN1/C(=N/C2=O)Nc2ccc(N3CCN(CCNc4cc(F)c([C@H]5CCC(=O)NC5=O)c(F)c4)[C@H](CC#N)C3)cc21]

The titled compound was prepared in a manner similar to that in Example 421.

¹H NMR (500 MHz, DMSO) δ 12.54 (s, 1H), 10.83 (s, 1H), 8.42 (s, 1H), 7.92 (s, 1H), 7.56 (s, 1H), 7.40 (d, J=8.7 Hz, 1H), 7.16 (s, 1H), 6.90 (d, J=8.4 Hz, 1H), 6.29 (d, J=11.9 Hz, 2H), 6.07 (s, 1H), 4.37-4.32 (m, 1H), 4.16-4.12 (m, 1H), 4.04-3.89 (m, 3H), 3.73 (s, 3H), 3.22-3.14 (m, 3H), 3.14-2.98 (m, 4H), 2.94-2.91 (m, 1H), 2.88-2.71 (m, 4H), 2.63-2.559 (m, 2H), 2.55 (s, 3H), 2.22-2.18 (m, 1H), 2.06-1.89 (m, 4H), 1.45 (s, 1H), 1.18-1.11 (m, 2H), 0.82-0.71 (m, 3H); [M+H]⁺=820.6.

### Example 443: (R)-3-(4-(((1S,3R)-3-(4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)cyclobutyl)amino)-2,6-difluorophenyl)piperidine-2,6-dione

### Step 1: tert-butyl 4-(3-(((benzyloxycarbonyl)amino)cyclobutyl)piperazine-1-carboxylate

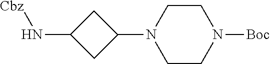 [SMILES: CC(C)(C)OC(=O)N1CCN(C2CC(NC(=O)OCc3ccccc3)C2)CC1]

To a 100-mL round-bottomed flask equipped with a magnetic stir bar was added benzyl (3-oxocyclobutyl)carbamate (2.2 g, 10.0 mmol), tert-butyl piperazine-1-carboxylate (2.2 g, 12.0 mmol), NaBH(OAc)₃ (4.3 g, 20.0 mmol) and DCE (40 mL) at room temperature. After stirring for 5 h at 50° C., the reaction mixture was diluted with sat. aq. NaHCO₃(150 mL) and the layers were separated. The aqueous layer was extracted with DCM (3×50 mL). The combined organic layers were washed with brine (100 mL), dried over anhydrous Na₂SO₄, filtered and concentrated under reduced pressure. Purification by flash column chromatography (0-10% MeOH in DCM) afforded the product (3.3 g, 85%). [M+H]⁺=390.3.

### Step 2: tert-butyl 4-(3-aminocyclobutyl)piperazine-1-carboxylate

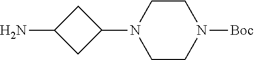 [SMILES: CC(C)(C)OC(=O)N1CCN(C2CC(N)C2)CC1]

To a 250-mL round-bottomed flask equipped with a magnetic stir bar was added tert-butyl 4-(3-(((benzyloxy)carbonyl)amino)cyclobutyl)piperazine-1-carboxylate (3.3 g, 8.5 mmol), Pd/C (wet, 10 wt %, 3.3 g) and MeOH (70 mL). The mixture was stirred at room temperature for 3 hours under hydrogen atmosphere (balloon). The mixture was then filtered by celite directly. The filtrate was concentrated in vacuum to afford the crude product (1.9 g, 88%) which was used without further purification. [M+H]⁺=256.2.

### Step 3: tert-butyl 4-((1r,3r)-3-((4-(2,6-bis(benzyloxy)pyridin-3-yl)-3,5-difluorophenyl)amino)cyclobutyl)piperazine-1-carboxylate

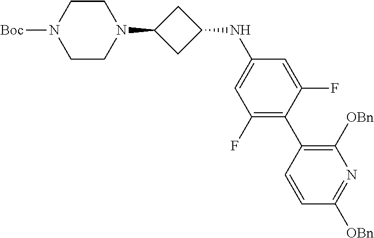 [SMILES: CC(C)(C)OC(=O)N1CCN([C@H]2C[C@H](Nc3cc(F)c(-c4ccc(OCc5ccccc5)nc4OCc4ccccc4)c(F)c3)C2)CC1]

A mixture of 2,6-bis(benzyloxy)-3-(4-bromo-2,6-difluorophenyl)pyridine (3.5 g, 7.2 mmol), tert-butyl 4-(3-aminocyclobutyl)piperazine-1-carboxylate (2.8 g, 10.8 mmol), CuI (305.6 mg, 1.6 mmol), L-proline (521.6 mg, 3.2 mmol), and K₃PO₄ (5.1 g, 24.0 mmol) in DMSO (100 mL) was heated to 100° C. for 15 hours under nitrogen atmosphere. After cooling to r.t, the reaction was diluted with EA (100 mL) and then washed with brine (300 mL×3), dried over Na₂SO₄, filtered and concentrated in vacuum. The residue was purified by silica gel column (PE:EA=1:1) to afford the mixture of cis and trans isomers (1.1 g, 22%). The trans product could be separated by chiral-HPLC (Lux Cellulose-2 4.6*50 mm 3 um, Hex (0.1% DEA):EtOH=95:5, 1.0 ml/min) and the title compound corresponded to peak B @ 4.452 min/254 nm (602 mg). [M+H]⁺=657.3.

### Step 4: tert-butyl 4-((1r,3r)-3-((4-(2,6-dioxopiperidin-3-yl)-3,5-difluorophenyl)amino)cyclobutyl)piperazine-1-carboxylate

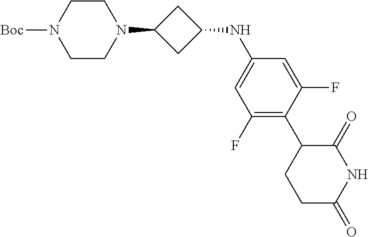 [SMILES: CC(C)(C)OC(=O)N1CCN([C@H]2C[C@H](Nc3cc(F)c(C4CCC(=O)NC4=O)c(F)c3)C2)CC1]

To a 100-mL round-bottomed flask equipped with a magnetic stir bar was added tert-butyl 4-((1r,3r)-3-((4-(2,6-bis(benzyloxy)pyridin-3-yl)-3,5-difluorophenyl)amino)cyclobutyl)piperazine-1-carboxylate (1.0 g, 1.5 mmol), Pd/C (wet, 10 wt %, 1.0 g) and DMF/iPrOH (50 mL, 1:1). The mixture was stirred at 50° C. for 15 hours under hydrogen atmosphere (balloon). The mixture was then filtered by celite directly. The filtrate was concentrated in vacuum to afford the crude product (690 mg, 96%) which was used without further purification. [M+H]⁺=479.2.

### Step 5: 3-(2,6-difluoro-4-(((1r,3r)-3-(piperazin-1-yl)cyclobutyl)amino)phenyl)piperidine-2,6-dione

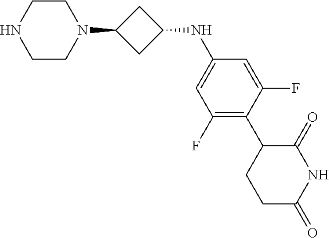 [SMILES: O=C1CCC(c2c(F)cc(N[C@H]3C[C@H](N4CCNCC4)C3)cc2F)C(=O)N1]

A solution of tert-butyl 4-((1r,3r)-3-((4-(2,6-dioxopiperidin-3-yl)-3,5-difluorophenyl)amino)cyclobutyl)piperazine-1-carboxylate (690 mg, 1.4 mmol) in TFA/DCM (30 mL, 1:4) was stirred at r.t. for 2 h. The reaction was concentrated in vacuum to afford the crude product (500 mg) which was used without further purification. [M+H]⁺=379.2.

### Step 6: (R)-3-(4-(((1S,3R)-3-(4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1·2)-cyclopropanacyclodecaphane-5⁶-yl)piperazin-1-yl)cyclobutyl)amino)-2,6-difluorophenyl)piperidine-2,6-dione

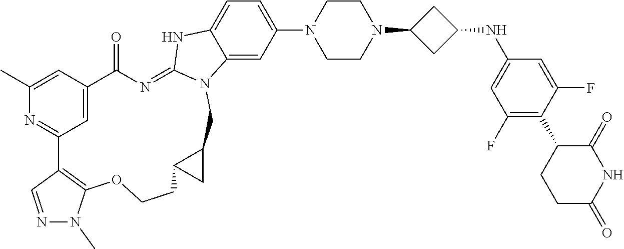 [SMILES: Cc1cc2cc(n1)-c1cnn(C)c1OCC[C@H]1C[C@@H]1CN1/C(=N/C2=O)Nc2ccc(N3CCN([C@H]4C[C@H](Nc5cc(F)c([C@H]6CCC(=O)NC6=O)c(F)c5)C4)CC3)cc21]

A mixture of 3-(2,6-difluoro-4-(((1r,3r)-3-(piperazin-1-yl)cyclobutyl)amino)phenyl)piperidine-2,6-dione (151 mg, 0.40 mmol), (7¹S,7²R,E)-5⁶-bromo-1¹,2⁶-dimethyl-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphan-3-one (167 mg, 0.33 mmol), NaOtBu (222 mg, 2.3 mmol), Pd₂(dba)₃ (60 mg, 0.07 mmol) and RuPhos (62 mg, 0.13 mmol) in DMA (10 mL) was stirred for 2 h at 100° C. under nitrogen atmosphere. The mixture was allowed to cool down to room temperature and diluted with DCM (50 mL), which was washed with brine (50 mL×3), dried over anhydrous Na₂SO₄, filtered, and concentrated under reduced pressure. Purification by silica gel column chromatography (0-10% MeOH in DCM) afforded the mixture of two isomers (120 mg, 45%). The title product could be separated by chiral-HPLC (CHIRALPAK IE-3 4.6*50 mm 3 um. MtBE (0.1% FA):(MeOH:DCM=1:1)=30:70, 1.0 ml/min) and the title compound corresponded to peak B @ 1.763 min/254 nm (35 mg). ¹H NMR (500 MHz, DMSO) δ 12.44 (s, 1H), 10.84 (s, 1H), 8.71 (s, 1H), 7.88 (s, 1H), 7.50 (s, 1H), 7.35 (d, J=8.5 Hz, 1H), 7.06 (s, 1H), 6.90 (d, J=9.0 Hz, 1H), 6.63 (d, J=5.0 Hz, 1H), 6.12 (d, J=11.5 Hz, 2H), 4.32-4.22 (m, 3H), 4.12-4.09 (m, 1H), 3.99 (dd, J=12.5, 5.0 Hz, 1H), 3.83-3.76 (m, 3H), 3.75 (s, 3H), 3.23-3.14 (m, 4H), 2.95-2.90 (m, 1H), 2.81-2.73 (m, 1H), 2.56 (s, 3H), 2.50-2.45 (m, 3H), 2.32-2.27 (m, 2H), 2.13-2.03 (m, 2H), 1.98-1.93 (m, 3H), 1.50-1.42 (m, 1H), 1.38-1.31 (m, 1H), 1.17-1.13 (m, 1H), 0.55-0.53 (m, 1H), 0.41-0.35 (m, 1H). [M+H]⁺=805.6

### Example 444: (R)-3-(2,6-difluoro-4-(((1R,3R)-3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)cyclobutyl)amino)phenyl)piperidine-2,6-dione

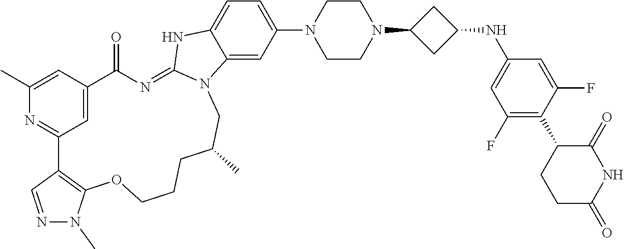 [SMILES: Cc1cc2cc(n1)-c1cnn(C)c1OCCC[C@@H](C)CN1/C(=N/C2=O)Nc2ccc(N3CCN([C@H]4C[C@H](Nc5cc(F)c([C@H]6CCC(=O)NC6=O)c(F)c5)C4)CC3)cc21]

The titled compound was prepared in a manner similar to that in Example 421. ¹H NMR (500 MHz, DMSO) δ 12.52 (s, 1H), 10.84 (s, 1H), 8.42 (s, 1H), 7.92 (s, 1H), 7.56 (s, 1H), 7.38 (d, J=8.5 Hz, 1H), 7.17 (s, 1H), 6.91 (d, J=8.0 Hz, 1H), 6.64 (s, 1H), 6.12 (d, J=12.0 Hz, 2H), 4.41-4.34 (m, 1H), 4.18-4.14 (m, 1H), 4.03-3.95 (m, 3H), 3.81-3.77 (m, 1H), 3.73 (s, 3H), 3.24-3.14 (m, 4H), 2.98-2.88 (m, 1H), 2.85-2.75 (m, 2H), 2.55 (s, 3H), 2.50-2.44 (m, 4H), 2.36-2.20 (m, 3H), 2.11-1.89 (m, 6H), 1.50-1.41 (m, 1H), 1.25-1.21 (m, 1H), 0.82 (d, J=6.5 Hz, 3H). [M+H]⁺=807.6

Example 444 also was obtained through the method below:

### (R)-3-(2,6-difluoro-4-(((1R,3R)-3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)cyclobutyl)amino)phenyl)piperidine-2,6-dione

### Step 1: tert-butyl 4-(3-(((benzyloxy)carbonyl)amino)cyclobutyl)piperazine-1-carboxylate

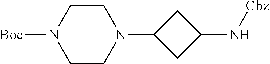 [SMILES: CC(C)(C)OC(=O)N1CCN(C2CC(NC(=O)OCc3ccccc3)C2)CC1]

To a 250-mL round-bottomed flask equipped with a magnetic stir bar was added benzyl (3-oxocyclobutyl)carbamate (2.2 g, 10.0 mmol), tert-butyl piperazine-1-carboxylate (2.2 g, 12.0 mmol), NaBH(OAc)₃ (4.2 g, 20.0 mmol) and DCE (100 mL). After stirring for 6 h at 50° C., the reaction mixture was diluted with sat. aq. NaHCO₃(150 mL) and the layers were separated. The aqueous layer was extracted with DCM (3×70 mL). The combined organic layers were washed with brine (150 mL), dried over anhydrous Na₂SO₄, filtered and concentrated under reduced pressure. Purification by flash column chromatography (0-7% MeOH in DCM) afforded the product (3.8 g, 98%). [M+H]⁺=390.0.

### Step 2: tert-butyl 4-(3-aminocyclobutyl)piperazine-1-carboxylate

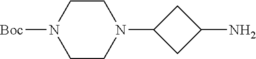 [SMILES: CC(C)(C)OC(=O)N1CCN(C2CC(N)C2)CC1]

To the solution of tert-butyl 4-(3-(((benzyloxy)carbonyl)amino)cyclobutyl)piperazine-1-carboxylate (3.8 g, 9.8 mmol) in 100 mL MeOH was added Pd/C (4.0 g, 10 wt. %, wet). The mixture was stirred at rt for 3 hours under hydrogen atmosphere (balloon). After LCMS showed the reaction was completed, the mixture was filtered by celite directly, and the solid was washed by MeOH (20 mL) and DCM (100 mL). The filtrate was concentrated in vacuum to afford the crude product (2.8 g), which was used without further purification.[M+H]-=255.9.

### Step 3: tert-butyl 4-((1r,3r)-3-((4-(2,6-bis(benzyloxy)pyridin-3-yl)-3,5-difluorophenyl)amino)cyclobutyl)piperazine-1-carboxylate

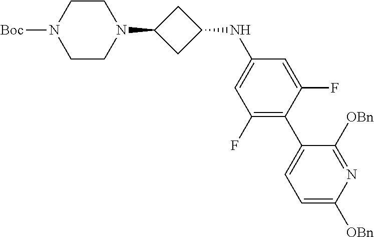 [SMILES: CC(C)(C)OC(=O)N1CCN([C@H]2C[C@H](Nc3cc(F)c(-c4ccc(OCc5ccccc5)nc4OCc4ccccc4)c(F)c3)C2)CC1]

The solution of 2,6-bis(benzyloxy)-3-(4-bromo-2,6-difluorophenyl)pyridine (3.5 g, 7.2 mmol), tert-butyl 4-(3-aminocyclobutyl)piperazine-1-carboxylate (2.8 g, 10.8 mmol), K₃PO₄ (5.1 g, 24.0 mmol), CuI (306 mg, 1.6 mmol) and L-Proline (522 mg, 3.2 mmol) in 100 mL DMSO was stirred at 100° C. for 16 hours under nitrogen atmosphere. The mixture was diluted with EtOAc and filtered. The filtrate was washed with brine (300 mL×3), dried over anhydrous Na₂SO₄, filtered and concentrated under reduced pressure. The crude was purified by flash column chromatography (0-7% MeOH in DCM) and SFC (Lux Cellulose-2 4.6*50 mm 3 um, Hex (0.1% DEA):EtOH=95:5) and the title compound corresponded to peak B @ii 4.452 min/254 nm (602 mg, 13%). [M+H]⁺=657.1.

### Step 4: tert-butyl 44(r,3r)-3-((4-(2,6-dioxopiperidin-3-yl)-3,5-difluorophenyl)amino)cyclobutyl)piperazine-1-carboxylate

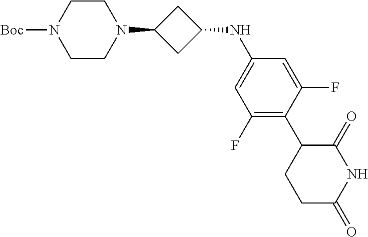 [SMILES: CC(C)(C)OC(=O)N1CCN([C@H]2C[C@H](Nc3cc(F)c(C4CCC(=O)NC4=O)c(F)c3)C2)CC1]

To the solution of tert-butyl 4-((1r,3r)-3-((4-(2,6-bis(benzyloxy)pyridin-3-yl)-3,5-difluorophenyl)amino)cyclobutyl)piperazine-1-carboxylate (1.0 g, 1.5 mmol) in 50 mL DMF/iPrOH was added Pd/C (1.0 g, 10% w.t.). The mixture was stirred at 50° C. for 16 hours under H₂ atmosphere. After LCMS showed the reaction was completed, the mixture was filtered through celite directly, and the solid was washed by MeOH (20 mL) and DCM (100 mL). The filtrate was concentrated in vacuum to afford the crude product (604 mg), which was used without further purification.[M+H]⁺=478.9.

### Step 5: 3-(2,6-difluoro-4-(((1r,3r)-3-(piperazin-1-yl)cyclobutyl)amino)phenyl)piperidine-2,6-dione

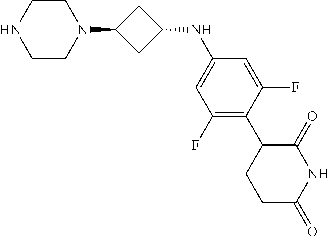 [SMILES: O=C1CCC(c2c(F)cc(N[C@H]3C[C@H](N4CCNCC4)C3)cc2F)C(=O)N1]

To a 100-mL round-bottomed flask equipped with a magnetic stir bar was added tert-butyl 4-((1r,3r)-3-((4-(2,6-dioxopiperidin-3-yl)-3,5-difluorophenyl)amino)cyclobutyl)piperazine-1-carboxylate (604 mg, 1.3 mmol), DCM (40 mL) and TFA (10 mL). After stirring for 1 h at room temperature, the reaction mixture was concentrated under reduced pressure to afford the product as TFA salt (720 mg), which was used without further purification. [M+H]⁺=378.9.

### Step 6: (R)-3-(2,6-difluoro-4-(((1R,3R)-3-(44(R,E)-1¹,2⁶,7-trimethyl-3-oxo-5²,5³-dihydro-1¹H,5¹H-1l-oxa-4-aza-5(2,1)-benzo[d]imidazola-2(2,4)-pyridina-1(4,5)-pyrazolacycloundecaphane-5⁶-piperazine-1-yl)cyclobutyl)amino)phenyl)piperidine-2,6-dione

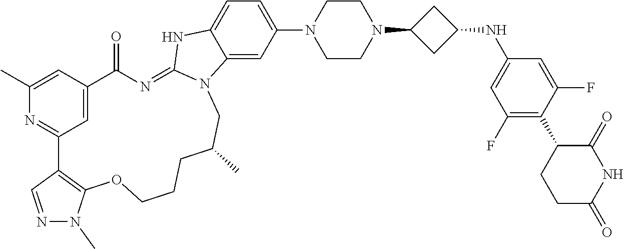 [SMILES: Cc1cc2cc(n1)-c1cnn(C)c1OCCC[C@@H](C)CN1/C(=N/C2=O)Nc2ccc(N3CCN([C@H]4C[C@H](Nc5cc(F)c([C@H]6CCC(=O)NC6=O)c(F)c5)C4)CC3)cc21]

To a mixture of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (150 mg, 0.3 mmol), 3-(2,6-difluoro-4-(((1r,3r)-3-(piperazin-1-yl)cyclobutyl)amino)phenyl)piperidine-2,6-dione (136 mg, 0.36 mmol), Pd₂(dba) (55 mg, 0.06 mmol), Ruphos (56 mg, 0.12 mmol), NaOBu (201 mg, 2.1 mmol) in DMA (10 mL) was stirred at 100° C. for 3 hrs. After cooling to r.t, the reaction was quenched with sat. NH₄Cl solution (50 mL), the resulting mixture was extracted with DCM (20 mL×3). The combined organic phase was washed with brine (20 mL), dried over Na₂SO₄, filtered and concentrated in vacuum. The residue was purified by silica gel column (DCM:CH₃OH=20:1) and SFC (CHIRALPAK IE-3, MtBE (0.1% FA): (MeOH:DCM=1:1)=30:70) and the title compound corresponded to peak A @ 1.759 min/254 nm (30 mg, 12%). ¹H NMR (500 MHz, DMSO) δ 12.52 (s, 1H), 10.84 (s, 1H), 8.42 (s, 1H), 7.92 (s, 1H), 7.56 (s, 1H), 7.38 (d, J=10.0 Hz, 1H), 7.17 (s, 1H), 6.91 (d, J=9.0 Hz, 1H), 6.64 (s, 1H), 6.12 (d, J=12.0 Hz, 2H), 4.39-4.34 (m, 1H), 4.16-4.14 (m, 1H), 4.00-3.95 (m, 3H), 3.84-3.76 (m, 1H), 3.73 (s, 3H), 3.25-3.11 (m, 3H), 3.01-2.89 (m, 1H), 2.83-2.74 (m, 2H), 2.55 (s, 3H), 2.50-2.42 (m, 5H), 2.37-2.18 (m, 3H), 2.01-1.92 (m, 6H), 1.48-1.41 (m, 1H), 1.27-1.19 (m, 1H), 0.82 (d, J=6.5 Hz, 3H), [M+H]⁺=807.6.

### Example 445: (R)-3-(2,6-difluoro-4-(((1 S,3S)-3-(4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)cyclobutyl)amino)phenyl)piperidine-2,6-dione

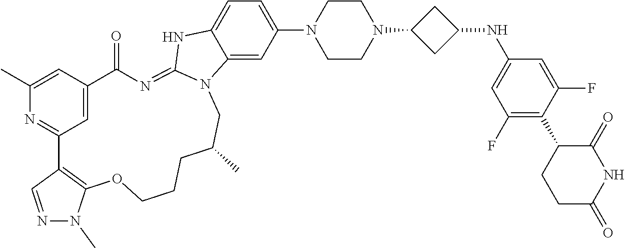 [SMILES: Cc1cc2cc(n1)-c1cnn(C)c1OCCC[C@@H](C)CN1/C(=N/C2=O)Nc2ccc(N3CCN([C@H]4C[C@@H](Nc5cc(F)c([C@H]6CCC(=O)NC6=O)c(F)c5)C4)CC3)cc21]

The titled compound was prepared in a manner similar to that in Example 421.

¹H NMR (500 MHz, DMSO) δ 12.51 (s, 1H), 10.83 (s, 1H), 8.42 (s, 1H), 7.92 (s, 1H), 7.55 (s, 1H), 7.37 (d, J=8.7 Hz, 1H), 7.15 (s, 1H), 6.89 (d, J=9.0 Hz, 1H), 6.49 (d, J=7.0 Hz, 1H), 6.18 (d, J=11.8 Hz, 2H), 4.37 (s, 1H), 4.14 (d, J=10.9 Hz, 1H), 4.01-3.95 (m, 3H), 3.73 (s, 3H), 3.58 (d, J=7.2 Hz, 1H), 3.31-3.27 (m, 2H), 3.16 (s, 4H), 2.86-2.71 (m, 2H), 2.59 (s, 1H), 2.55 (s, 3H), 2.45 (s, 4H), 2.22 (s, 1H), 2.06 (d, J=8.2 Hz, 1H), 2.02-1.89 (m, 3H), 1.66 (d, J=9.7 Hz, 2H), 1.45 (s, 1H), 0.81 (d, J=6.5 Hz, 4H). [M+H]⁺=807.2.

### Example 446: (R)-3-(2,6-difluoro-4-(4-(2-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)ethyl)piperazin-1-yl)phenyl)piperidine-2,6-dione

### Step 1: (R,E)-5⁶-((E)-2-ethoxyvinyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

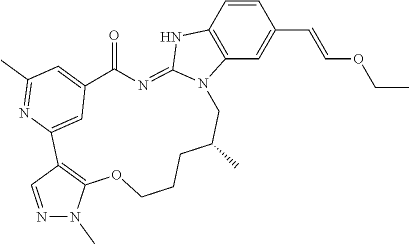 [SMILES: CCO/C=C/c1ccc2c(c1)N1C[C@H](C)CCCOc3c(cnn3C)-c3cc(cc(C)n3)C(=O)/N=C/1N2]

To a stirred solution of (R,E)-5⁶-bromo-1¹,2⁶,7-trimethyl-5²,5′-dihydro-1¹H,5¹H-11-oxa-4-aza-5(2,1)-benzo[d]imidazola-2(2,4)-pyridina-1(4,5)-pyrazolacycloundecaphan-3-one (0.3 g, 0.59 mmol) in dioxane (20 mL) and water (4 mL) were added (E)-2-(2-ethoxyvinyl)-4,4,5,5-tetramethyl-1,3,2-dioxaborolane (0.17 g, 0.88 mmol), Pd(dppf)Cl₂ (43 mg, 0.06 mmol) and K₂CO₃ (162 mg, 1.18 mmol) at room temperature under nitrogen atmosphere. The resulting mixture was stirred for 16 h at 100° C. under nitrogen atmosphere. The mixture was allowed to cool down to room temperature. The organic phase was concentrated under reduced pressure. The residue was diluted with EtOAc (10 mL), washed with water (3×4 mL) and brine (4 mL). The organic layer was dried over anhydrous Na₂SO₄, filtered and concentrated under reduced pressure. The residue was purified by silica gel column chromatography, eluted with DCM/MeOH (10:1) to afford the product (164 mg, 55.8%); [M+H]⁺=501.1.

### Step 2: (R,E)-2-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)acetaldehyde

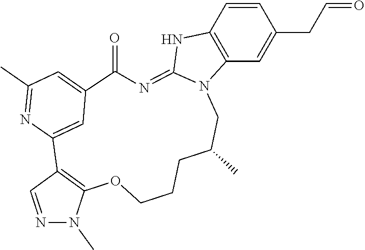 [SMILES: Cc1cc2cc(n1)-c1cnn(C)c1OCCC[C@@H](C)CN1/C(=N/C2=O)Nc2ccc(CC=O)cc21]

To a 100-mL round-bottomed flask equipped with a magnetic stir bar was added (R,E)-5⁶-((E)-2-ethoxyvinyl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (0.165 g, 0.33 mmol) and FA (10 mL) was stirred at room temperature for 2 h. Then the mixture was concentrated and DCM (100 mL) was added. The organic layer was extracted with DCM (3×5 mL). The combined organic layers were washed with brine (50 mL), dried over anhydrous Na₂SO₄, filtered and concentrated under reduced pressure. Purification by flash column chromatography (0-10% MeOH in DCM) afforded the product (90 mg, 57.9%). [M+H]⁺=473.5.

### Step 3 (R)-3-(2,6-difluoro-4-(4-(2-((R,E)-1¹,2⁶,7-trimethyl-3-oxo-5²,5¹-dihydro-1¹H,5¹H-11-oxa-4-aza-5(2,1)-benzo[d]imidazola-2(2,4)-pyridina-1(4,5)-pyrazolacycloundecaphane-5⁶-yl)ethyl)piperazin-1-yl)phenyl)piperidine-2,6-dione

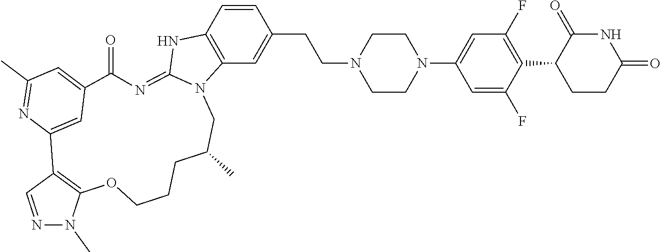 [SMILES: Cc1cc2cc(n1)-c1cnn(C)c1OCCC[C@@H](C)CN1/C(=N/C2=O)Nc2ccc(CCN3CCN(c4cc(F)c([C@H]5CCC(=O)NC5=O)c(F)c4)CC3)cc21]

To a solution of (R,E)-2-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)acetaldehyde (90 mg, 0.19 mmol), (R)-3-(2,6-difluoro-4-(piperazin-1-yl)phenyl)piperidine-2,6-dione (64 mg, 0.21 mmol) in DCM (10 mL) was added STAB (121 mg, 0.57 mmol). Then the mixture was stirred at room temperature for 1 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=10:1) to afford (R)-3-(2,6-difluoro-4-(4-(2-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)ethyl)piperazin-1-yl)phenyl)piperidine-2,6-dione (4.15 mg, 2.85%).

¹H NMR (500 MHz, DMSO) δ 12.65 (s, 1H), 10.87 (s, 1H), 8.43 (s, 1H), 7.92 (s, 1H), 7.56 (s, 1H), 7.51 (s, 1H), 7.44 (d, J=8.1 Hz, 1H), 7.12 (d, J=7.9 Hz, 1H), 6.66 (s, 1H), 6.64 (s, 1H), 4.40-4.31 (m, 1H), 4.24-4.14 (m, 1H), 4.10-4.03 (m, 1H), 4.02-3.97 (m, 1H), 3.96-3.88 (m, 1H), 3.73 (s, 3H), 3.24-3.16 (m, 4H), 2.92-2.86 (m, 2H), 2.84-2.74 (m, 2H), 2.68-2.59 (m, 3H), 2.59-2.55 (m, 6H), 2.52 (d, J=1.8 Hz, 1H), 2.27-2.15 (m, 1H), 2.14-2.03 (m, 1H), 2.03-1.86 (m, 3H), 1.50-1.41 (m, 1H), 0.88-0.77 (m, 3H); [M+H]⁺=766.6.

### Example 447: (R)-3-(2,6-difluoro-4-(4-((S)-2-(hydroxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

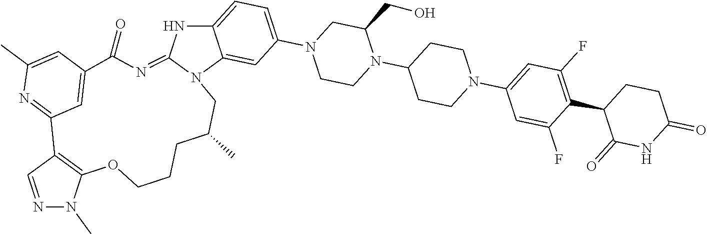 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@H](CO)C3)cc21]

The titled compound was prepared in a manner similar to that in Example 421.

¹H NMR (500 MHz, DMSO) δ 12.49 (s, 1H), 10.87 (s, 1H), 8.42 (s, 1H), 7.92 (s, 1H), 7.55 (s, 1H), 7.37 (d, J=8.7 Hz, 1H), 7.08 (s, 1H), 6.87 (d, J=8.5 Hz, 1H), 6.61-6.59 (m, 2H), 4.63 (s, 1H), 4.36-4.32 (m, 1H), 4.15-4.12 (m, 1H), 4.05-4.02 (m, 1H), 4.02-3.90 (m, 2H), 3.83-3.81 (m, 2H), 3.73-3.69 (m, 3H), 3.66-3.62 (m, 1H), 3.57-3.54 (m, 1H), 3.29-3.22 (m, 3H), 2.98-2.96 (m, 3H), 2.78-2.75 (m, 6H), 2.64-2.61 (m, 1H), 2.55-2.51 (m, 3H), 2.29-2.14 (m, 1H), 2.14-2.03 (m, 1H), 2.02-1.85 (m, 4H), 1.76-1.68 (m, 1H), 1.68-1.56 (m, 1H), 1.51-1.38 (m, 2H), 0.82 (d, J=6.4 Hz, 3H); [M+H]⁺=851.6.

### Example 448: (R)-3-(2,6-difluoro-4-(4-((R)-2-oxo-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)piperazin-1-yl)phenyl)piperidine-2,6-dione

### Step 1: methyl (R)-2-methyl-6-(1-methyl-5-((4-methyl-5-((2-nitro-5-(7-oxo-1,4-dioxa-8-azaspiro[4.5]decan-8-yl)phenyl)amino)pentyl)oxy)-1H-pyrazol-4-yl)isonicotinate

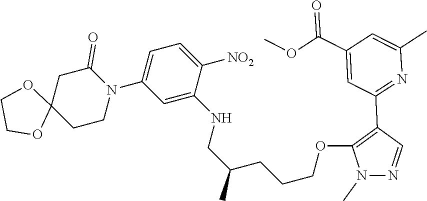 [SMILES: COC(=O)c1cc(C)nc(-c2cnn(C)c2OCCC[C@@H](C)CNc2cc(N3CCC4(CC3=O)OCCO4)ccc2[N+](=O)[O-])c1]

To the solution of methyl (R)-2-(5-((5-((5-bromo-2-nitrophenyl)amino)-4-methylpentyl)oxy)-1-methyl-1H-pyrazol-4-yl)-6-methylisonicotinate (2.0 g, 3.7 mmol), 1,4-dioxa-8-azaspiro[4.5]decan-7-one (1.2 g, 7.4 mmol), CuI (350 mg, 1.8 mmol) and K₃PO₄ (2.7 g, 12.8 mmol) in 40 mL DMSO was added N¹,N²-dimethylethane-1,2-diamine (161 mg, 1.8 mmol). The mixture was stirred at 100° C. for 5 hours under N₂. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (DCM:MeOH=100:1-20:1) to afford the product (900 mg, 39.5%). [M+H]⁺=623.5.

### Step 2: methyl (R)-2-(5-((5-((2-amino-5-(7-oxo-1,4-dioxa-8-azaspiro[4.5]decan-8-yl)phenyl)amino)-4-methylpentyl)oxy)-1-methyl-1H-pyrazol-4-yl)-6-methylisonicotinate

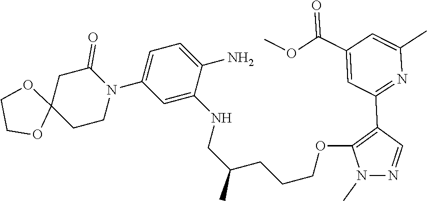 [SMILES: COC(=O)c1cc(C)nc(-c2cnn(C)c2OCCC[C@@H](C)CNc2cc(N3CCC4(CC3=O)OCCO4)ccc2N)c1]

The title compound was prepared in a procedure similar to Example 9 step 4. [M+H]⁺=593.3.

### Step 3: methyl (R)-2-(5-((5-(2-amino-6-(7-oxo-1,4-dioxa-8-azaspiro[4.5]decan-8-yl)-1H-benzo[d]imidazol-1-yl)-4-methylpentyl)oxy)-1-methyl-1H-pyrazol-4-yl)-6-methylisonicotinate

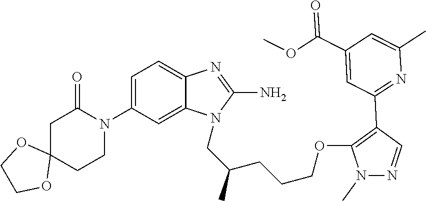 [SMILES: COC(=O)c1cc(C)nc(-c2cnn(C)c2OCCC[C@@H](C)Cn2c(N)nc3ccc(N4CCC5(CC4=O)OCCO5)cc32)c1]

The title compound was prepared in a procedure similar to Example 9 step 5. [M+H]⁺=618.5.

### Step 4: (R,E)-1¹,2⁶,7-trimethyl-5⁶-(7-oxo-1,4-dioxa-8-azaspiro[4.5]decan-8-yl)-5²,5³-dihydro-1¹H,5¹H-11-oxa-4-aza-5(2,1-benzo[d]imidazola-2(2,4)-pyridina-1(4,5)-pyrazolacycloundecaphan-3-one

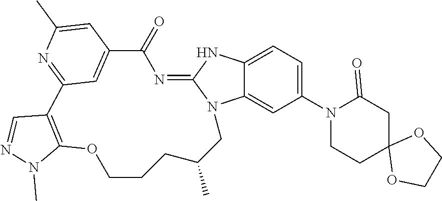 [SMILES: Cc1cc2cc(n1)-c1cnn(C)c1OCCC[C@@H](C)CN1/C(=N/C2=O)Nc2ccc(N3CCC4(CC3=O)OCCO4)cc21]

To a solution of methyl (R)-2-(5-((5-(2-amino-6-(7-oxo-1,4-dioxa-8-azaspiro[4.5]decan-8-yl)-1H-benzo[d]imidazol-1-yl)-4-methylpentyl)oxy)-1-methyl-1H-pyrazol-4-yl)-6-methylisonicotinate (600 mg, 0.96 mmol) in DMF (30 mL) was added LiHMDS (1.0 mol/L in THF, 3.0 mL) dropwise at 0° C. The mixture was stirred at 0° C. for 20 minutes. The mixture was quenched with NH₄Cl/water and extracted by DCM. The organic layer was dried over anhydrous Na₂SO₄, filtered and concentrated under reduced pressure to give the crude residue, which was purified by silica column chromatography (DCM:MeOH=20:1) to afford the product (310 mg, 54.5%). [M+H]⁺=586.5.

### Step 5: (R,E)-1-(1¹,2⁶,7-trimethyl-3-oxo-5²,5³-dihydro-1¹H,5¹H-1-oxa-4-aza-5(2,1)-benzo[d]imidazola-2(2,4)-pyridina-1(4,5)-pyrazolacycloundecaphane-5⁶-yl)piperidine-2,4-dione

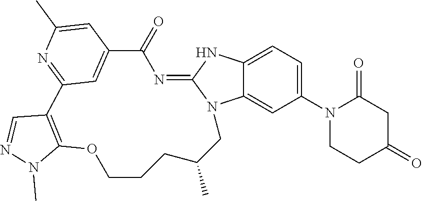 [SMILES: Cc1cc2cc(n1)-c1cnn(C)c1OCCC[C@@H](C)CN1/C(=N/C2=O)Nc2ccc(N3CCC(=O)CC3=O)cc21]

(R,E)-1¹,2⁶,7-trimethyl-5⁶-(7-oxo-1,4-dioxa-8-azaspiro[4.5]decan-8-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (310 mg, 0.53 mmol) was placed in 100 mL round bottom flask with a magnetic stir bar. Then 9 mL 8N HCl aqueous was added. The mixture was stirred at room temperature for 2 hours. The mixture was added dropwise to sat. aq. NaHCO₃ solution and finally pH=6-7. The liquid was extracted with DCM and separated. The organic phase was concentrated in vacuum and purified with combiflash (DCM:MeOH=20:1) to afford the title compound (285 mg, 99.3% yield). [M+H]⁺=542.5.

### Step 6: (R)-3-(2,6-difluoro-4-(4-((R)-2-oxo-1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)piperazin-1-yl)phenyl)piperidine-2,6-dione

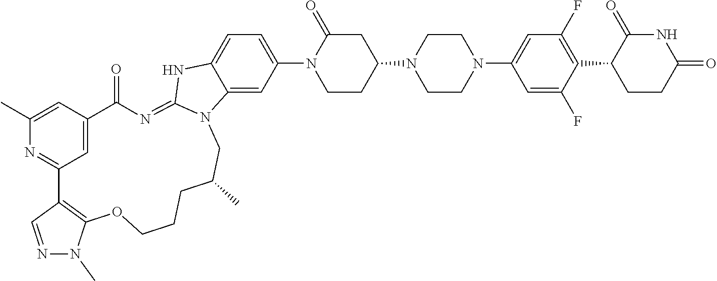 [SMILES: Cc1cc2cc(n1)-c1cnn(C)c1OCCC[C@@H](C)CN1/C(=N/C2=O)Nc2ccc(N3CC[C@@H](N4CCN(c5cc(F)c([C@H]6CCC(=O)NC6=O)c(F)c5)CC4)CC3=O)cc21]

To a solution of (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidine-2,4-dione (140 mg, 0.26 mmol), (R)-3-(2,6-difluoro-4-(piperazin-1-yl)phenyl)piperidine-2,6-dione (120 mg, 0.35 mmol) and DIEA (90 mg, 0.7 mmol) in DCE (10 mL) was added STAB (165 mg, 0.78 mmol). Then the mixture was stirred at 50° C. for 48 hours. H₂O (10 mL) was added and the resulting mixture was extracted with DCM (15 mL×3). The combined organic phase was washed with brine (10 mL), dried over Na₂SO₄, filtered and concentrated in vacuum. The residue was purified by silica gel column (DCM:CH₃OH=15:1) to give the racemate, which was separated by Prep-Chiral-HPLC with following condition:(Column: (R,R)Whelk-O1, 2 cm×25 cm, 5 um; Flow rate: 20 mL/min; Gradient: 10% to 50% in 2.0 min, hold 1.0 min at 50%, Injection Volume: 5 uL) to afford the title product (3.0 mg, 1.4%). ¹H NMR (500 MHz, DMSO) δ_{H} 12.76 (s, 1H), 10.88 (s, 1H), 8.43 (s, 1H), 7.92 (s, 1H), 7.56 (d, J=9.8 Hz 2H), 7.51 (d, J=8.5 Hz, 1H), 7.12 (d, J=8.4 Hz, 1H), 6.67 (d, J=12.7 Hz, 2H), 4.36 (s, 1H), 4.19 (d, J=10.9 Hz 1¹H), 4.07 (dd, J=12.4, 5.2 Hz, 1H), 4.00 (s, 1H), 3.90 (d, J=13.3 Hz, 1H), 3.75-3.70 (m, 4H), 3.62 (s, 1H), 3.26-3.20 (m, 4H), 2.93 (s, 1H), 2.85-2.75 (m, 2H), 2.72-2.60 (m, 6H), 2.56 (s, 3H), 2.36 (s, 1H), 2.24-2.06 (m, 3H), 2.02-1.85 (m, 4H), 1.44 (s, 1H), 0.82 (d, J=6.4 Hz, 3H). [M+H]⁺=835.6.

### Example 449: (R)-3-(2,6-difluoro-4-(((1R,3R)-3-((4-((R,E)-1¹,2⁶,7-trimethyl-3-oxo-5²,5³-dihydro-1′1H,5¹H-11-oxa-4-aza-5(2,1)-benzo[d]imidazola-2(2,4)-pyridina-1(4,5)-pyrazolacycloundecaphane-5⁶-yl)piperazin-1-yl)methyl)cyclobutyl)amino)phenyl)piperidine-2,6-dione

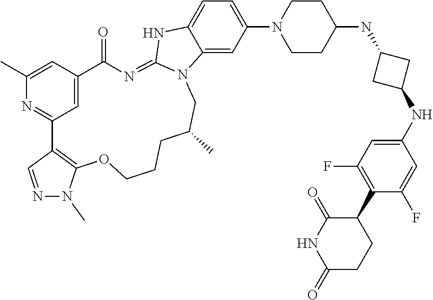 [SMILES: Cc1cc2cc(n1)-c1cnn(C)c1OCCC[C@@H](C)CN1/C(=N/C2=O)Nc2ccc(N3CCC(N[C@H]4C[C@H](Nc5cc(F)c([C@H]6CCC(=O)NC6=O)c(F)c5)C4)CC3)cc21]

The title compound was prepared in a procedure similar to that in Example 421. ¹H NMR (500 MHz, DMSO) δ_{H} 12.51 (s, 1H), 10.83 (s, 1H), 8.42 (s, 1H), 7.92 (s, 1H), 7.56 (s, 1H), 7.36 (s, 1H), 7.14 (s, 1H), 6.88 (d, J=7.6 Hz, 1H), 6.56 (d, J=6.0 Hz, 1H), 6.13 (d, J=11.9 Hz, 2H), 4.36 (d, J=4.3 Hz, 1H), 4.14 (d, J=11.4 Hz, 1H), 4.02-3.88 (m, 4H), 3.73 (s, 3H), 3.16 (s, 4H), 2.86-2.71 (m, 2H), 2.58-2.52 (m, 7H), 2.48 (s, 3H), 2.25-1.90 (m, 10H), 1.44 (d, J=6.2 Hz, 1H), 0.81 (d, J=6.4 Hz, 3H). [M+H]⁺=821.6.

### Example 450: (R)-3-(2,6-difluoro-4-(3-oxo-4-(1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)piperazin-1-yl)phenyl)piperidine-2,6-dione

### Step 1: tert-butyl 4-((2-(((benzyloxy)carbonyl)amino)ethyl)amino)piperidine-1-carboxylate

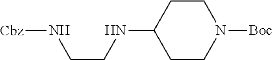 [SMILES: CC(C)(C)OC(=O)N1CCC(NCCNC(=O)OCc2ccccc2)CC1]

To a solution of tert-butyl 4-oxopiperidine-1-carboxylate (3.1 g, 15.0 mmol), benzyl (2-aminoethyl)carbamate (3.0 g, 15.0 mmol) and AcOH (1.5 g, 22.5 mmol) in DCE (100 mL) was added STAB (6.3 g, 30.0 mmol). Then the mixture was stirred at RT for 16 hours. The reaction was quenched with NaHCO₃/water (100 mL) and extracted with DCM (150 mL×2). The combined organic phase was washed with brine (100 mL), dried over Na₂SO₄, filtered and concentrated in vacuum. The residue was purified by silica gel column (DCM:CH₃OH=15:1), to afford the title product (5.5 g, 93.3%). [M+H]⁺=378.5.

### Step 2: tert-butyl 4-(N-(2-(((benzyloxy)carbonyl)amino)ethyl)-2-chloroacetanido)piperidine-1-carboxylate

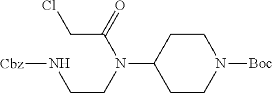 [SMILES: CC(C)(C)OC(=O)N1CCC(N(CCNC(=O)OCc2ccccc2)C(=O)CCl)CC1]

To a solution of tert-butyl 4-((2-(((benzyloxy)carbonyl)amino)ethyl)amino)piperidine-1-carboxylate (5.5 g, 14.5 mmol) and DIEA (5.6 g, 43.5 mmol) in DCM (150 mL) was added 2-chloroacetyl chloride (2.0 g, 17.4 mmol) cooled to 0° C. Then the mixture was stirred at RT for 3 hours. The reaction was quenched with water (100 mL) and extracted with DCM (150 mL×2). The combined organic phase was washed with brine (100 mL×1), dried over Na₂SO₄, filtered and concentrated in vacuum. The residue was purified by silica gel column (DCM:CH₃OH=20:1), to afford the title product (6.0 g, 90.9%). [M+H]⁺=454.5.

### Step 3: benzyl 4-(1-(tert-butoxycarbonyl)piperidin-4-yl)-3-oxopiperazine-1-carboxylate

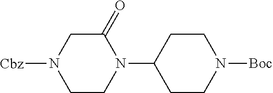 [SMILES: CC(C)(C)OC(=O)N1CCC(N2CCN(C(=O)OCc3ccccc3)CC2=O)CC1]

To a solution of tert-butyl 4-(N-(2-(((benzyloxy)carbonyl)amino)ethyl)-2-chloroacetamido)piperdine-1-carboxylate (6.0 g, 13.2 mmol) in DMF (80 mL) was added NaH (1.0 g, 26.4 mmol) at RT. Then the mixture was stirred at RT for 0.5 hour. The reaction was quenched with NH₄Cl/water (100 mL) and extracted with EA (150 mL×2). The combined organic phase was washed with brine (100 mL×2), dried over Na₂SO₄, filtered and concentrated in vacuum. The residue was purified by silica gel column (DCM:CH₃OH=20:1), to afford the title product (5.3 g, 96.3%). [M+H]⁺=418.5.

### Step 4: tert-butyl 4-(2-oxopiperazin-1-yl)piperidine-1-carboxylate

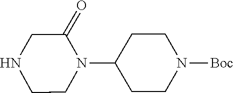 [SMILES: CC(C)(C)OC(=O)N1CCC(N2CCNCC2=O)CC1]

To the solution of benzyl 4-(I-(tert-butoxycarbonyl)piperidin-4-yl)-3-oxopiperazine-1-carboxylate (5.3 g, 12.7 mmol) in THF (100 mL) was added Pd/C (1.5 g, 10 wt. %, wet). The resulting mixture was stirred at RT for 3 hours under hydrogen atmosphere (balloon). After LCMS showed the reaction was completed, the mixture was filtered by celite and washed with DCM. The filtrate was concentrated in vacuum to afford the desired product (3.4 g, 94.4%). [M+H]⁺=284.5.

### Step 5: tert-butyl 4(4-(4-(2,6-bis(benzyloxy,)pyridin-3-yl)-3,5-difluorophenyl)-2-oxopiperazin-1-ylpiperidine-1-carboxylate

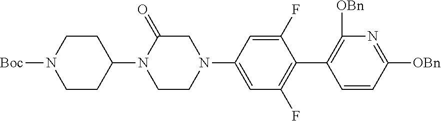 [SMILES: CC(C)(C)OC(=O)N1CCC(N2CCN(c3cc(F)c(-c4ccc(OCc5ccccc5)nc4OCc4ccccc4)c(F)c3)CC2=O)CC1]

To the solution of 2,6-bis(benzyloxy)-3-(4-bromo-2,6-difluorophenyl)pyridine (2.0 g, 4.1 mmol), tert-butyl 4-(2-oxopiperazin-1-yl)piperidine-1-carboxylate (1.8 g, 6.3 mmol) and Cs₂CO₃ (2.7 g, 8.2 mmol) in 100 mL 1,4-dioxane were added G3 RuPhos Pd (690 mg, 0.82 mmol) and RuPhos (383 mg, 0.82 mmol). The mixture was stirred at 100° C. for 16 hours. After LCMS showed the reaction was completed, the mixture was filtered through a celite pad and washed with DCM. The filtrate was concentrated under reduced pressure to give the crude residue, which was purified by silica column chromatography (PE:EA=50:1-1:1) to afford the product (2.6 g, 91.5%). [M+H]⁺=685.5.

### Step 6: tert-butyl 4-(4-(4-(2,6-dioxopiperidin-3-yl)-3,5-difluorophenyl)-2-oxopiperazin-1-yl)piperidine-1-carboxylate

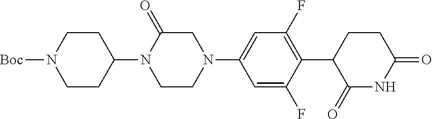 [SMILES: CC(C)(C)OC(=O)N1CCC(N2CCN(c3cc(F)c(C4CCC(=O)NC4=O)c(F)c3)CC2=O)CC1]

To the solution of tert-butyl 4-(4-(4-(2,6-bis(benzyloxy)pyridin-3-yl)-3,5-difluorophenyl)-2-oxopiperazin-1-yl)piperidine-1-carboxylate (1.1 g, 1.6 mmol) in 15 mL DMF and 15 mL i-PrOH, Pd/C (0.5 g, 10 wt. %, wet) was added. The mixture was stirred at 50° C. for 24 hours under hydrogen atmosphere (balloon). After LCMS showed the reaction was completed, the mixture was cooled to room temperature and filtered by celite directly. The filtrate was concentrated in vacuum to afford the desired product (780 mg, 95.8%). [M+H]⁺=507.5

### Step 7: 3-(2,6-difluoro-4-(3-oxo-4-(piperidin-4-yl)piperazin-1-yl)phenyl)piperidine-2,6-dione

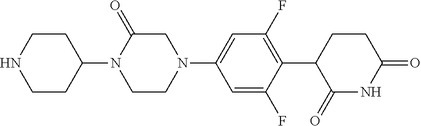 [SMILES: O=C1CCC(c2c(F)cc(N3CCN(C4CCNCC4)C(=O)C3)cc2F)C(=O)N1]

A solution of tert-butyl 4-(4-(4-(2,6-dioxopiperidin-3-yl)-3,5-difluorophenyl)-2-oxopiperazin-1-yl)piperidine-1-carboxylate (780 mg, 1.5 mmol) in HCl/1,4-dioxane (20 mL) was stirred at room temperature for 1 hour, the reaction mixture was concentrated under reduced pressure to afford the product (625 mg), which was used without further purification. [M+H]⁺=407.5

### Step 8: (R)-3-(2,6-difluoro-4-(3-oxo-441-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperidin-4-yl)piperazin-1-yl)phenyl)piperidine-2,6-dione

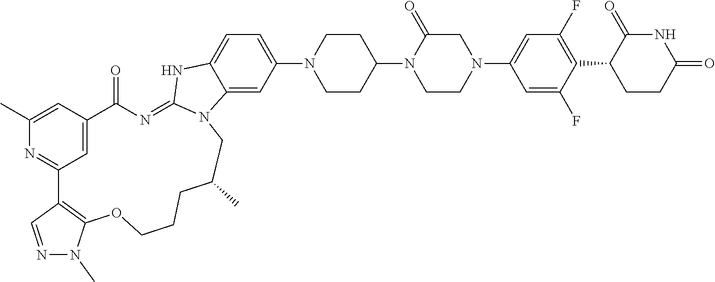 [SMILES: Cc1cc2cc(n1)-c1cnn(C)c1OCCC[C@@H](C)CN1/C(=N/C2=O)Nc2ccc(N3CCC(N4CCN(c5cc(F)c([C@H]6CCC(=O)NC6=O)c(F)c5)CC4=O)CC3)cc21]

To the solution of (R,E)-5⁶-bromo-1¹,2⁶,7-trimethyl-5²,5³-dihydro-1′1H,5¹H-li-oxa-4-aza-5(2,1)-benzo[d]imidazola-2(2,4)-pyridina-1(4,5)-pyrazolacycloundecaphan-3-one (100 mg, 0.2 mmol), 3-(2,6-difluoro-4-(3-oxo-4-(piperidin-4-yl)piperazin-1-yl)phenyl)piperidine-2,6-dione (105 mg, 0.24 mmol) and t-BuONa (56 mg, 0.6 mmol) in 6 mL DMA were added Pd₂(dba)₃ (36 mg, 0.04 mmol) and RuPhos (36 mg, 0.08 mmol). The mixture was stirred at 100° C. for 1.5 hour under N₂. After LCMS showed the reaction was completed, the reaction was quenched with NH₄Cl/H₂O (15 mL) and extracted by DCM (15 mL×3). The combined organic phase was washed with brine (15 mL), dried over Na₂SO₄, filtered and concentrated in vacuum. The residue was purified by silica gel column (DCM:CH₃OH=15:1) to give the racemate, which was further purified by Prep chiral-HPLC with following condition:(Column: CHIRALPAK IF, 2 cm×25 cm, 5 um; Flow rate: 20 mL/min; Gradient: 10% to 50% in 2.0 min, hold 1.0 min at 50%, Injection Volume: 5 uL) to afford the title product (38 mg, 23.2%). ¹H NMR (500 MHz, DMSO) δ_{H} 12.52 (s, 1H), 10.88 (s, 1H), 8.43 (s, 1H), 7.92 (s, 1H), 7.56 (s, 1H), 7.38 (d, J=8.7 Hz, 1H), 7.19 (s, 1H), 6.93 (dd, J=8.8, 1.7 Hz, 1H), 6.64 (d, J=12.4 Hz, 2H), 4.45 (t, J=12.2 Hz, 1H), 4.39-4.33 (m, 1H), 4.14 (d, J=11.1 Hz, 1H), 4.07 (dd, 1=12.6, 4.9 Hz, 1H), 3.99 (t, J=11.7 Hz, 2H), 3.88 (s, 2H), 3.81 (t, J=12.9 Hz, 2H), 3.73 (s, 3H), 3.50 (d, J=5.7 Hz, 2H), 3.42 (d, J=5.3 Hz, 2H), 2.85-2.75 (m, 4H), 2.56-2.52 (m, 4H), 2.22 (s, 1H), 2.14-2.05 (m, 1H), 2.02-1.85 (m, 5H), 1.68 (d, J=11.1 Hz, 2H), 1.45 (s, 1H), 0.83 (d, J=6.5 Hz, 3H). [M+H]⁺=835.5.

### Example 451: (R)-3-(2,6-difluoro-4-(3-(((S)-2-(hydroxymethyl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)methyl)azetidin-1-yl)phenyl)piperidine-2,6-dione

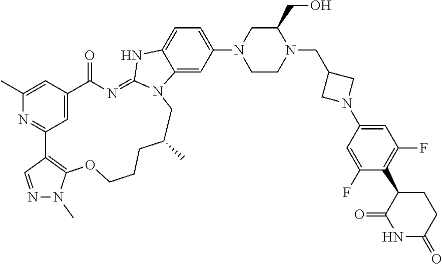 [SMILES: Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)[C@H](CO)C3)cc21]

The titled compound was prepared in a manner similar to that in Example 421.

¹H NMR (500 MHz, DMSO) δ 12.52 (s, 1H), 10.85 (s, 1H), 8.42 (s, 1H), 7.92 (s, 1H), 7.56 (s, 1H), 7.38 (d, J=8.7 Hz, 1H), 7.13 (s, 1H), 6.89 (d, J=8.6 Hz, 1H), 6.12 (d, J=11.2 Hz, 2H), 4.62 (t, J=5.2 Hz, 1H), 4.37 (d, J=4.8 Hz, 1H), 4.15-4.12 (m, 1H), 4.07-3.90 (m, 5H), 3.73 (s, 3H), 3.70-3.64 (m, 1H), 3.50-3.45 (m, 4H), 3.32-3.29 (m, 3H), 3.15-3.05 (m, 1H), 3.01-2.86 (m, 3H), 2.84-2.72 (m, 3H), 2.55-2.49 (m, 4H), 2.42-2.39 (m, 1H), 2.22-2.18 (m, 1H), 2.13-2.05 (m, 1H), 1.95-1.91 (m, 3H), 1.45 (s, 1H), 0.82 (d, J=6.5 Hz, 3H); [M+H]⁺=837.6.

### Example 452: 2-((S)-1-(1-(4-((R)-2,6-dioxopiperidin-3-yl)-3,5-difluorophenyl)piperidin-4-yl)-4-((R,E)-1¹,2⁶,7-trim ethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-2-yl)acetonitrile

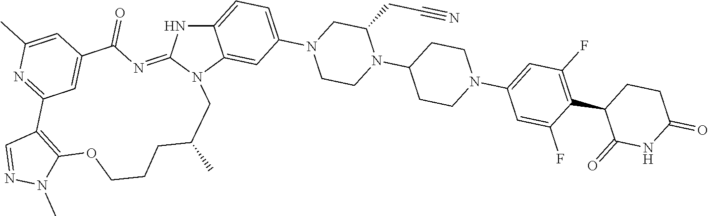 [SMILES: Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@@H](CC#N)C3)cc21]

The titled compound was prepared in a manner similar to that in Example 421.

¹H NMR (500 MHz, DMSO) δ 12.47 (s, 1H), 10.87 (s, 1H), 8.42 (s, 1H), 7.92 (s, 1H), 7.56 (s, 1H), 7.40 (d, J=8.6 Hz, 1H), 7.14 (s, 1H), 6.89 (d, J=7.1 Hz, 1H), 6.65 (d, J=12.8 Hz, 2H), 4.36-4.32 (d, 1H), 4.16-4.13 (m, 1H), 4.06-4.04 (m, 1H), 4.02-4.00 (m, 2H), 3.82-3.79 (m, 2H), 3.73 (s, 3H), 3.30-3.25 (m, 3H), 3.19-3.00 (m, 4H), 2.88-2.69 (m, 8H), 2.55-2.52 (m, 3H), 2.22-2.18 (m, 1H), 2.15-2.05 (m, 1H), 2.02-1.83 (m, 4H), 1.79-1.76 (m, 1H), 1.59-1.54 (m, 1H), 1.44-1.41 (m, 2H), 0.82 (d, J=6.5 Hz, 3H): [M+H]⁺=860.6.

### Example 453: (R)-3-(2,6-difluoro-4-(4-(4-((R,E)-2⁶-methoxy-1¹,7-dimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

### Step 1: methyl 2-(5-hydroxy-1-methyl-1H-pyrazol-4-yl-6-methoxyisonicotinate

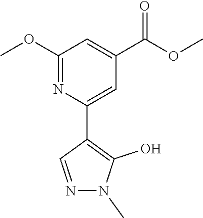

To a solution of methyl 2-chloro-6-methoxyisonicotinate (3.0 g, 15.0 mmol) and 2-methylpyrazol-3-ol (3.0 g, 30.0 mmol) in ANISOLE (50 mL) was added Na₂CO₃ (3.9 g, 37.5 mmol) and Pd(dppf)Cl₂ (1.1 g, 1.5 mmol). The mixture was stirred at 130° C. under N₂ for 12 hours and turned to brown suspension. The reaction mixture was then cooled to 20° C. and was filtered through a celite pad was washed with DCM. The filtrate was concentrated under reduced pressure to give the crude residue, which was purified by silica column chromatography (DCM:MeOH=10:1) to afford the product (1.9 g, 48.7%). [M+H]⁺=264.5.

### Step 2: (R)-5-((2-amino-5-bromophenyl)amino)-4-methylpentan-1-ol

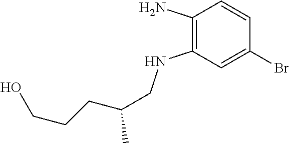 [SMILES: C[C@H](CCCO)CNc1cc(Br)ccc1N]

The title compound was prepared in a procedure similar to that in Example 9 step 4. [M+H]⁺=287.5.

### Step 3: (R)-5-(6-bromo-2-imino-2,3-dihydro-1H-benzo[d]imidazol-1-yl)-4-methylpentan-1-ol

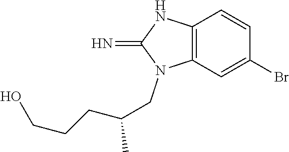 [SMILES: C[C@H](CCCO)Cn1c(=N)[nH]c2ccc(Br)cc21]

The title compound was prepared in a procedure similar to that in Example 9 step 5. [M+H]⁺=312.5.

### Step 4: methyl (R)-2-(54(5-(6-bromo-2-imino-2,3-dihydro-1H-benzo[d]imidazol-1-yl)-4-methylpentyl)oxy)-1-methyl-1H-pyrazol-4-yl)-6-methoxyisonicotinate

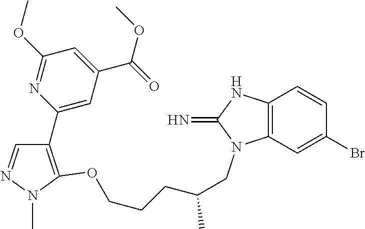 [SMILES: COC(=O)c1cc(OC)nc(-c2cnn(C)c2OCCC[C@@H](C)Cn2c(=N)[nH]c3ccc(Br)cc32)c1]

The title compound was prepared in a procedure similar to that in Example 391 step 10. [M+H]⁺=557.5.

### Step 5: (R,E)-5⁶-bromo-2⁶-methoxy-1,7-dimethyl-5²,5³-dihydro-1¹H,5¹H-11-oxa-4-aza-5(2,1)-benzo[d]imidazola-2(2,4)-pyridina-1(4,5-pyrazolacycloundecaphan-3-one

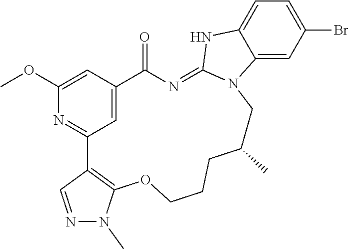 [SMILES: COc1cc2cc(n1)-c1cnn(C)c1OCCC[C@@H](C)CN1/C(=N/C2=O)Nc2ccc(Br)cc21]

The title compound was prepared in a procedure similar to that in Example 391 step 13. [M+H]⁺=525.5.

### Step 6: tert-butyl (R,E)-4-(2⁶-methoxy-1¹,7-dimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazine-1-carboxylate

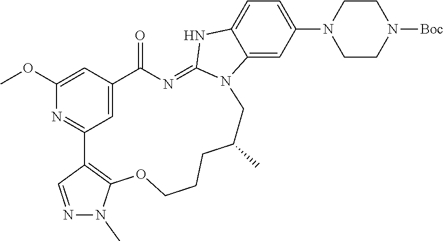 [SMILES: COc1cc2cc(n1)-c1cnn(C)c1OCCC[C@@H](C)CN1/C(=N/C2=O)Nc2ccc(N3CCN(C(=O)OC(C)(C)C)CC3)cc21]

The title compound was prepared in a procedure similar to that in Example 324 step 1. [M+H]⁺=631.5.

### Step 7: (R,E)-2⁶-methoxy-1¹,7-dimethyl-56-(pyrazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one

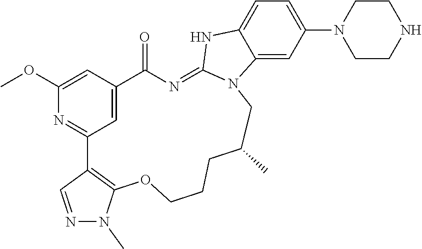 [SMILES: COc1cc2cc(n1)-c1cnn(C)c1OCCC[C@@H](C)CN1/C(=N/C2=O)Nc2ccc(N3CCNCC3)cc21]

The title compound was prepared in a procedure similar to that in Example 324 step 2. [M+H]⁺=531.5.

### Step 8: (R)-3-(2,6-difluoro-4-(4-(4-((R,E)-2⁶-methoxy-1¹,7-dimethyl-3-oxo-5²,5³-dihydro-1H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)phenyl)piperidine-2,6-dione

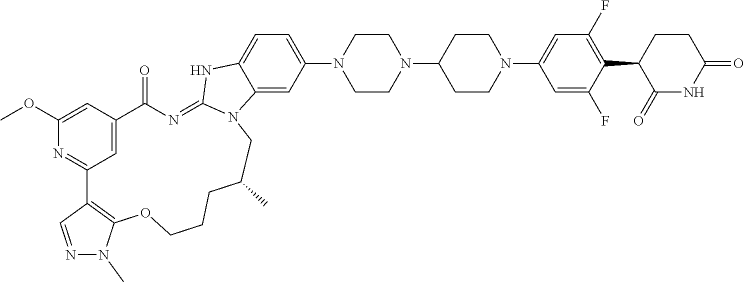 [SMILES: COc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21]

The title compound was prepared in a procedure similar to that in Example 324 step 3. ¹H NMR (500 MHz, DMSO) δ_{H} 12.53 (s, 1H), 10.87 (s, 1H), 8.21 (s, 1H), 7.96 (s, 1H), 7.36 (d, J=8.7 Hz, 1H), 7.14 (s, 1H), 7.08 (s, 1H), 6.89 (d, J=8.3 Hz, 1H), 6.64 (d, J=12.8 Hz, 2H), 4.37 (d, J=4.6 Hz, 1H), 4.13 (d, J=11.2 Hz, 1H), 4.05 (dd, J=12.5, 5.0 Hz, 1H), 4.00-3.92 (m, 5H), 3.81 (d, J=12.4 Hz, 2H), 3.73 (s, 3H), 3.16 (s, 4H), 2.82-2.74 (m, 4H), 2.68 (s, 4H), 2.45 (t, J=11.0 Hz, 2H), 2.21 (s, 1H), 2.12-2.05 (m, 1H), 2.00-1.85 (m, 5H), 1.54-1.40 (m, 3H), 0.81 (d, J=6.5 Hz, 3H). [M+H]⁺=837.6.

### Example 454: (R)-3-(2,6-difluoro-4-(4-((1-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5-yl)azetidin-3-yl)amino)piperidin-1-yl)phenyl)piperidine-2,6-dione

### Step 1:(R,E)-5⁶-(3-hydroxyazetidin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

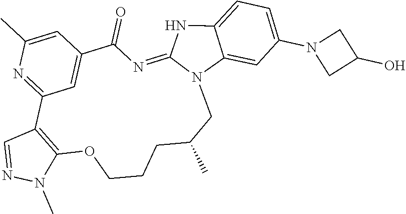 [SMILES: Cc1cc2cc(n1)-c1cnn(C)c1OCCC[C@@H](C)CN1/C(=N/C2=O)Nc2ccc(N3CC(O)C3)cc21]

To a solution of (R,E)-5⁶-bromo-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (200 mg, 0.39 mmol) in dioxane (5 mL) were added azetidin-3-ol (86.09 mg, 1.18 mmol), Pd₂(dba)₃ (35.68 mg, 0.039 mmol), Ruphos (36.11 mg, 0.078 mmol), Cs₂CO₃(383 mg, 1.17 mmol), The resulting solution was stirred for 2 h at 100° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with DCM/MeOH (10:1) to afford (R,E)-5⁶-(3-hydroxyazetidin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 50.78%) [M+H]⁺=502.5

### Step 2: (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)azetidin-3-yl methanesulfonate

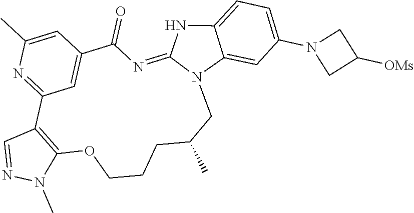 [SMILES: Cc1cc2cc(n1)-c1cnn(C)c1OCCC[C@@H](C)CN1/C(=N/C2=O)Nc2ccc(N3CC(OS(C)(=O)=O)C3)cc21]

To a solution of (R,E)-5⁶-(3-hydroxyazetidin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 0.19 mmol), TEA (40.35, 0.398) in DCM (4 mL) were added MsCl (34.2 mg, 0.29 mmol), The resulting solution was stirred for 2 h at rt, the reaction was quenched with water and extracted with DCM. The combined organic layers were washed with brine, dried over anhydrous Na₂SO₄. After filtration, the filtrate was concentrated under reduced pressure. The residue was purified by silica gel column chromatography, eluted with DCM/MeOH (10:1) to afford (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)azetidin-3-yl methanesulfonate (110 mg, 95.2%). [M+H]⁺=580.2.

### Step 3:(R,E)-5⁶-(3-aminoazetidin-1-yl)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

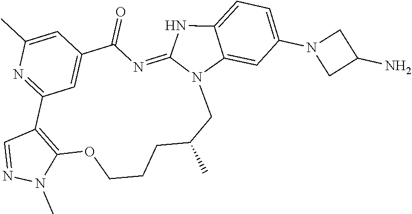 [SMILES: Cc1cc2cc(n1)-c1cnn(C)c1OCCC[C@@H](C)CN1/C(=N/C2=O)Nc2ccc(N3CC(N)C3)cc21]

To a solution of (R,E)-1-(1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)azetidin-3-yl methanesulfonate (100 mg, 0.19 mmol) in 7M NH₃(g) in MeOH solution (10 mL). The mixture was stirred in a flask at 60° C. for 5 h. The mixture was evaporated in vacuum to afford the crude product (80 mg, 92.6%), which was used for next step without further purification.

### Step 3: (R)-3-(2,6-difluoro-4-(4-((14(R,E)-1¹,2⁶,7-trimethyl-3-oxo-5²,5³-dihydro-1¹H,5¹H-11-oxa-4-aza-5(2, 1)-benzo[d]imidazola-2(2,4)-pyridina-1(4,5)-pyrazolacycloundecaphane-5⁶-yl)azetidin-3-yl)amino)piperidin-1-yl )phenyl)piperidine-2,6-dione

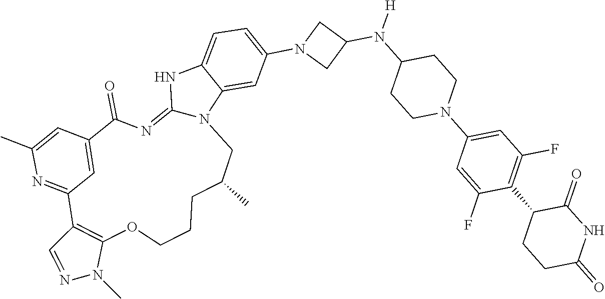 [SMILES: Cc1cc2cc(n1)-c1cnn(C)c1OCCC[C@@H](C)CN1/C(=N/C2=O)Nc2ccc(N3CC(NC4CCN(c5cc(F)c([C@H]6CCC(=O)NC6=O)c(F)c5)CC4)C3)cc21]

The titled compound was prepared in a manner similar to that in Example 421.

¹H NMR (500 MHz, DMSO-d₆): δ 12.45 (s, 1H), 10.86 (s, 1H), 8.42 (s, 1H), 7.92 (s, 1H), 7.55 (s, 1H), 7.33 (d, J=8.5 Hz, 1H), 6.62 (d, J=12.8 Hz, 3H), 6.35 (d, J=10.3 Hz, 1H), 4.40-4.33 (m, 2H), 4.17-4.08 (m, 3H), 4.04 (dd, J=12.7, 5.2 Hz, 1H), 4.01-3.95 (m, 1H), 3.89 (dd, J=14.8, 9.4 Hz, 1H), 3.82 (dt, J=9.3, 7.7 Hz, 1H), 3.73 (s, 3H), 2.81 (t, J=12.1 Hz, 4H), 2.70-2.62 (m, 2H), 2.55 (s, 3H), 2.21 (t, J=16.6 Hz, 1H), 2.06 (d, J=9.1 Hz, 1H), 1.95 (dd, J=17.1, 9.6 Hz, 4H), 1.80 (q, J=10.4 Hz, 2H), 1.47 (dd, J=33.0, 17.4 Hz, 3H), 1.30 (d, J=11.9 Hz, 3H), 0.81 (d, J=6.5 Hz, 3H). [M+H]⁺=807.4.

### Example 455: (R)-3-(4-(4-(3-(((71S,72R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)amino)azetidin-1-yl)piperidin-1-yl)-2,6-difluorophenyl)piperidine-2,6-dione

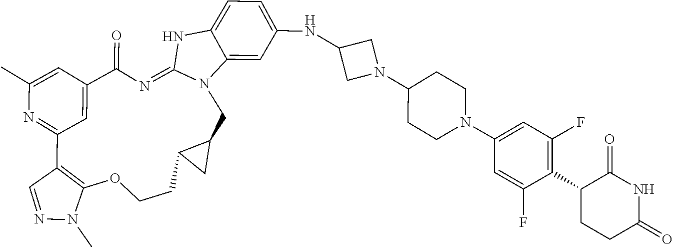 [SMILES: Cc1cc2cc(n1)-c1cnn(C)c1OCC[C@H]1C[C@@H]1CN1/C(=N/C2=O)Nc2ccc(NC3CN(C4CCN(c5cc(F)c([C@H]6CCC(=O)NC6=O)c(F)c5)CC4)C3)cc21]

The titled compound was prepared in a manner similar to that in Example 459. ¹H NMR (500 MHz, DMSO) δ 12.27 (s, 1H), 10.80 (s, 1H), 8.64 (s, 1H), 7.81 (s, 1H), 7.42 (s, 1H), 6.57 (d, J=12.9 Hz, 2H), 6.50 (s, 1H), 6.44 (d, J=8.6 Hz, 1H), 6.06 (s, 1H), 4.20-4.12 (m, 3H), 4.09-3.94 (m, 3H), 3.75-3.72 (m, 1H), 3.68 (s, 3H), 3.58 (s, 2H), 2.91-2.81 (m, 3H), 2.77-2.68 (m, 1H), 2.48-2.38 (m, 8H), 2.02-1.94 (m, 2H), 1.92-1.85 (m, 1H), 1.69 (s, 2H), 1.35 (d, J=7.0 Hz, 1H), 1.29-1.14 (m, 3H), 1.10-1.00 (m, 1H), 0.49-0.39 (m, 1H), 0.32-0.26 (m, 1H); [M+H]⁺=805.5.

### Example 456: 2-((2S)-1-(1-(6-(2,6-dioxopiperidin-3-yl)-2-methylpyridin-3-yl)piperidin-4-yl)-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-2-yl)acetonitrile

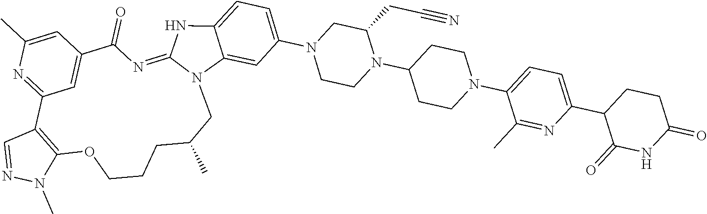 [SMILES: Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5C)CC4)[C@@H](CC#N)C3)cc21]

The titled compound was prepared in a manner similar to that in Example 324. ¹H NMR (500 MHz, DMSO) δ 12.54 (s, 1H), 10.78 (s, 1H), 8.43 (s, 1H), 7.92 (s, 1H), 7.56 (s, 1H), 7.39 (dd, J=14.2, 8.4 Hz, 2H), 7.16 (s, 1H), 7.11 (d, J=8.3 Hz, 1H), 6.90 (s, 1H), 5.76 (s, 2H), 4.37 (s, 1H), 4.17-4.11 (m, 1H), 4.05-3.93 (m, 2H), 3.91-3.86 (m, 1H), 3.73 (s, 3H), 3.21-3.04 (m, 6H), 2.89-2.62 (m, 8H), 2.59-2.55 (m, 4H), 2.41 (s, 3H), 2.28-2.16 (m, 2H), 2.16-2.04 (m, 1H), 2.03-1.83 (m, 4H), 1.74-1.71 (m, 1H), 1.60-1.56 (m, 1H), 1.51-1.41 (m, 1H), 0.83 (d, J=6.4 Hz, 3H); [M+H]⁺=839.6.

### Example 457: (R)-3-(4-(((1S,3R)-3-((4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)methyl)cyclobutyl)amino)-2,6-difluorophenyl)piperidine-2,6-dione

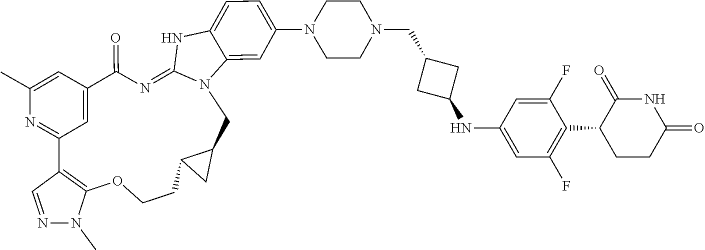 [SMILES: Cc1cc2cc(n1)-c1cnn(C)c1OCC[C@H]1C[C@@H]1CN1/C(=N/C2=O)Nc2ccc(N3CCN(C[C@H]4C[C@H](Nc5cc(F)c([C@H]6CCC(=O)NC6=O)c(F)c5)C4)CC3)cc21]

The title compound was prepared in a procedure similar to that in Example 449. ¹H NMR (500 MHz, DMSO) δ_{H} 12.43 (s, 1H), 10.83 (s, 1H), 8.71 (s, 1H), 7.88 (s, 1H), 7.50 (s, 1H), 7.34 (d, J=8.8 Hz, 1H), 7.05 (s, 1H), 6.89(d, J=8.6 Hz, 1H), 6.56(d, 1=6.1 Hz, 1H), 6.13 (d, J=11.9 Hz, 2H), 4.34-4.22 (m, 3H), 4.12 (d, J=6.9 Hz, 1H), 4.00-3.88 (m, 2H), 3.75 (s, 3H), 3.16 (s, 4H), 2.81-2.71 (m, 1H), 2.65-2.53 (m, 10H), 2.18-1.90 (m, 8H), 1.48-1.40 (m, 1H), 1.34 (s, 1H), 1.18-1.11 (m, 1H), 0.53 (s, 1H), 0.41-0.34 (m, 1H). [M+H]⁺=819.6.

### Example 458: (R)-3-(4-(4-(1-((7'S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperidin-4-yl)-3-oxopiperazin-1-yl)-2,6-difluorophenyl)piperidine-2,6-dione

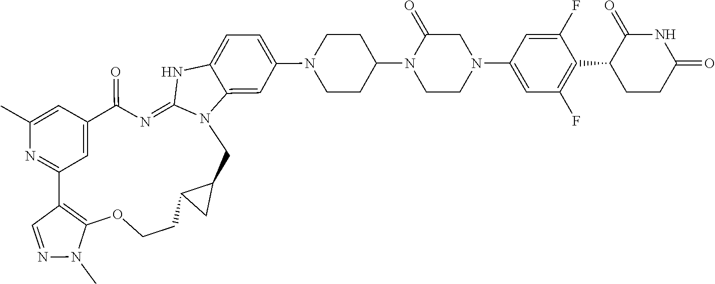 [SMILES: Cc1cc2cc(n1)-c1cnn(C)c1OCC[C@H]1C[C@@H]1CN1/C(=N/C2=O)Nc2ccc(N3CCC(N4CCN(c5cc(F)c([C@H]6CCC(=O)NC6=O)c(F)c5)CC4=O)CC3)cc21]

The title compound was prepared in a procedure similar to that in Example 450. ¹H NMR (500 MHz, DMSO) δ_{H} 12.45 (s, 1H), 10.88 (s, 1H), 8.71 (s, 1H), 7.88 (s, 1H), 7.50 (s, 1H), 7.35 (d, J=8.7 Hz, 1H), 7.09 (s, 1H), 6.95-6.90 (m, 1H), 6.65 (d, J=12.4 Hz, 2H), 4.45 (t, J=12.3 Hz, 1H), 4.36-4.23 (m, 3H), 4.15-4.03 (m, 2H), 3.89 (s, 2H), 3.81 (d, J=9.8 Hz, 2H), 3.75 (s, 3H), 3.50 (d, J=6.3 Hz, 2H), 3.42 (d, J=5.4 Hz, 2H), 2.80 (t, J=11.0 Hz, 3H), 2.65-2.54 (m, 4H), 2.10 (d, J=9.6 Hz, 2H), 2.00-1.87 (m, 3H), 1.68 (d, J=10.8 Hz, 2H), 1.50-1.40 (m, 1H), 1.34 (d, J=4.6 Hz, 1H), 1.18-1.12 (m, 1H), 0.58-0.50 (m, 1H), 0.42-0.35 (m, 1H). [M+H]⁺=833.6.

### Example 459: (R)-3-(2,6-difluoro-4-(4-(3-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidin-1-yl)piperidin-1-yl)phenyl)piperidine-2,6-dione

### Step 1: tert-buty (R,E)-3-((1¹,2⁶,7-trimethyl-3-oxo-5²,5³-dihydro-1¹H,5¹H-11-oxa-4-aza-5(2,1)-benzo[d]imidazola-2(2 ,4)-pyridina-1(4,5)-pyrazolacycloundecaphane-5⁶-yl)amino)azetidine-1-carboxylate

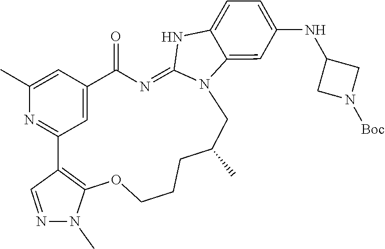 [SMILES: Cc1cc2cc(n1)-c1cnn(C)c1OCCC[C@@H](C)CN1/C(=N/C2=O)Nc2ccc(NC3CN(C(=O)OC(C)(C)C)C3)cc21]

The title compound was prepared in a procedure similar to that in Example 324 step 1. [M+H]⁺=601.5.

### Step 2: (R,E)-5⁶-(azetidin-3-ylamino)-1¹,2⁶,7-trimethyl-5²,5³-dihydro-1¹H,5¹H-11-oxa-4-aza-5(2,1)-benzo[d]imidazola-2(2,4)-pyridina-1(4,5)-pyrazolacycloundecaphan-3-one

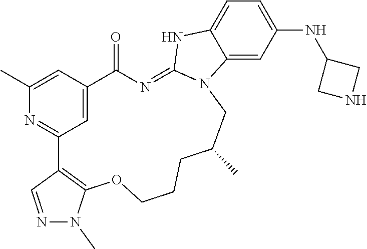 [SMILES: Cc1cc2cc(n1)-c1cnn(C)c1OCCC[C@@H](C)CN1/C(=N/C2=O)Nc2ccc(NC3CNC3)cc21]

The title compound was prepared in a procedure similar to that in Example 324 step 2. [M+H]⁺=501.5.

### Step 3: (R)-3-(2,6-difluoro-4-(4-(3-(((R,E)-1¹,2⁶,7-trimethyl-3-oxo-5²,5′-dihydro-1¹H,5¹H-11-oxa-4-aza-5(2,1)-benzo[d]imidazola-2(2,4)-pyridina-1(4,5)-pyrazolacycloundecaphane-5⁶-yl)amino)azetidin-1-yl)piperidin-1-yl)phenyl)piperidine-2,6-dione

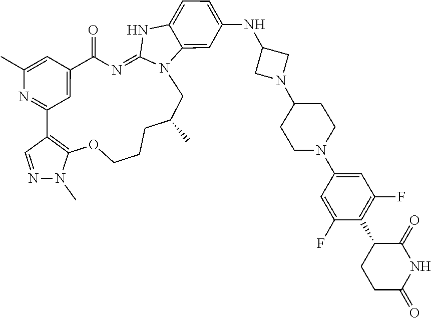 [SMILES: Cc1cc2cc(n1)-c1cnn(C)c1OCCC[C@@H](C)CN1/C(=N/C2=O)Nc2ccc(NC3CN(C4CCN(c5cc(F)c([C@H]6CCC(=O)NC6=O)c(F)c5)CC4)C3)cc21]

The title compound was prepared in a procedure similar to that in Example 324 step 3. ¹H NMR (500 MHz, DMSO) δ_{H} 12.41 (s, 1H), 10.87 (s, 1H), 8.41 (s, 1H), 7.92 (s, 1H), 7.55 (s, 1H), 7.27 (d, J=8.6 Hz, 1H), 6.62 (d, J=12.8 Hz, 3H), 6.52 (dd, J=8.6, 1.8 Hz, 1H), 6.07 (d, J=7.1 Hz, 1H), 4.36 (dd, J=9.0, 4.4 Hz, 1H), 4.14 (d, J=11.2 Hz, 1H), 4.08-3.96 (m, 3H), 3.85 (dd, J=13.3, 10.5 Hz, 1H), 3.78-3.72 (m, 5H), 3.60 (d, J=12.4 Hz, 2H), 2.92-2.73 (m, 6H), 2.56-2.53 (m, 4H), 2.30-2.20 (m, 2H), 2.09 (dd, J=13.0, 3.8 Hz, 1H), 2.02-1.87 (m, 3H), 1.72 (d, J=10.1 Hz, 2H), 1.45 (d, J=6.4 Hz, 1H), 1.30-1.21 (m, 2H), 0.82 (d, J=6.5 Hz, 3H). [M+H]⁺=807.6.

### Example 460: (R)-3-(3-fluoro-5-(4-(3-oxo-4-((R,E)-1¹,2⁶,7-trimethyl-3-oxo-5²,5³-dihydro-1¹H,5¹H-1¹-oxa-4-aza-5(2,1)-benzo[d]imidazola-2(2,4)-pyridina-1(4,5)-pyrazolacycloundecaphane-5⁶-yl)piperazin-1-yl)piperidin-1-yl)pyridin-2-yl)piperidine-2,6-dione

### Step 1: 8-(6-chloro-5-fluoropyridin-3-yl)-1,4-dioxa-8-azaspiro[4.5]decane

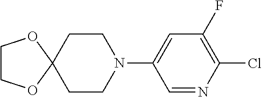 [SMILES: Fc1cc(N2CCC3(CC2)OCCO3)cnc1Cl]

To a solution of 5-bromo-2-chloro-3-fluoropyridine (2.0 g, 9.5 mmol) and 1,4-dioxa-8-azaspiro[4.5]decane (2.0 g, 14.25 mmol) in dioxane (150 mL) was added Pd₂(dba)₃ (871 mg, 0.95 mmol), Xantphos (887 mg, 1.9 mmol) and Cs₂CO₃ (9.2 g, 28.5 mmol) at room temperature. The suspension was degassed under vacuum and purged with N₂ three times. Then the mixture was stirred at 100° C. for 4 hours. The mixture was filtered and concentrated in vacuum. The residue was purified by silica gel column chromatography (PE:EA=100:0˜50: 50 gradient elution) to give the desired product (1.7 g, 65.6%). [M+H]⁺=273.4.

### Step 2: 8-(2′,6′-bis(benzyloxy)-3-fluoro-[2,3′-bipyridin]-5-yl)-1,4-dioxa-8-azaspiro[4.5]decane

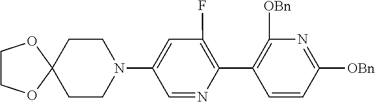 [SMILES: Fc1cc(N2CCC3(CC2)OCCO3)cnc1-c1ccc(OCc2ccccc2)nc1OCc1ccccc1]

To a solution of 8-(6-chloro-5-fluoropyridin-3-yl)-1,4-dioxa-8-azaspiro[4.5]decane (1.5 g, 5.5 mmol) and 2,6-bis(benzyloxy)-3-(4,4,5,5-tetramethyl-1,3,2-dioxaborolan-2-yl)pyridine (3.4 g, 8.25 mmol) in dioxane (50 mL) and water (6 mL) was added Pd(dppf)Cl₂ (871 mg, 0.95 mmol) and K₂CO₃ (2.27 g, 16.5 mmol) at room temperature. The suspension was degassed under vacuum and purged with N2 three times. Then the mixture was stirred at 100° C. overnight. The mixture was filtered and concentrated in vacuum. The residue was purified by silica gel column chromatography (PE:EA=100:0˜50: 50 gradient elution) to give the desired product (2.8 g, 96.5%). [M+H]⁺=528.4.

### Step 3: 3-(3-fluoro-5-(1,4-dioxa-8-azaspiro[4.5]decan-8-yl)pyridin-2-yl)piperidine-2,6-dione

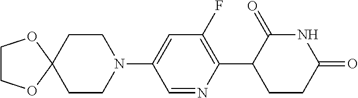 [SMILES: O=C1CCC(c2ncc(N3CCC4(CC3)OCCO4)cc2F)C(=O)N1]

To a solution of 8-(2′,6′-bis(benzyloxy)-3-fluoro-[2,3′-bipyridin]-5-yl)-1,4-dioxa-8-azaspiro[4.5]decane (2.7 g, 5.11 mmol) in DMF/i-PrOH (40 mL/20 mL) was added 10% Pd/C (2.7 g) at room temperature. And then the mixture was exchanged with H₂ three times and stirred under H₂ atmosphere at room temperature for 20 hours. Reaction was monitored by LC-MS. The mixture was filtered through a pad of Celite and washed with DMF (20 mL). The filtrate was concentrated under vacuum to obtain the title product (1.3 g, 73%). [M+H]⁺=350.5.

### Step 4: 3-(3-fluoro-5-(4-oxopiperidin-1-yl)pyridin-2-yl)piperidine-2,6-dione

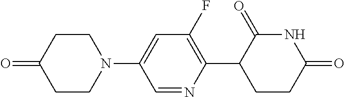 [SMILES: O=C1CCN(c2cnc(C3CCC(=O)NC3=O)c(F)c2)CC1]

A suspension of 3-(3-fluoro-5-(1,4-dioxa-8-azaspiro[4.5]decan-8-yl)pyridin-2-yl)piperidine-2,6-dione (500 mg, 1.8 mmol) in concentrated hydrochloric acid (6 mL) was stirred at room temperature for 2 hours. The reaction was quenched with saturated NaHCO₃(aq.) solution and extracted with DCM (2×30.0 mL). The combined organic layer was washed with brine (2×30.0 mL), dried over Na₂SO₄ and concentrated under vacuum to afford the crude product (350 mg, 80%). [M+H]⁺=306.3.

### Step 5: (R)-3-(3-fluoro-5-(4-(3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)piperidin-1-yl)pyridin-2-yl)piperidine-2,6-dione

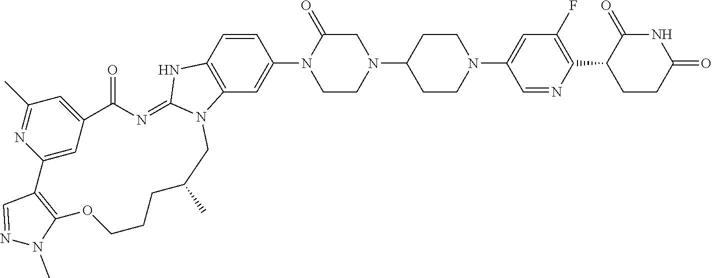 [SMILES: Cc1cc2cc(n1)-c1cnn(C)c1OCCC[C@@H](C)CN1/C(=N/C2=O)Nc2ccc(N3CCN(C4CCN(c5cnc([C@H]6CCC(=O)NC6=O)c(F)c5)CC4)CC3=O)cc21]

To a solution of (R,E)-1¹,2⁶,7-trimethyl-5⁶-(2-oxopiperazin-1-yl)-5²,5³-dihydro-1¹H,5¹H-11-oxa-4-aza-5(2,1)-benzo[d]imidazola-2(2,4)-pyridina-1(4,5)-pyrazolacycloundecaphan-3-one (100 mg, 0.189 mmol) and 3-(3-fluoro-5-(4-oxopiperidin-1-yl)pyridin-2-yl)piperidine-2,6-dione (75 mg, 0.245 mmol) in DCE (6 mL) was added sodium triacetoxyborohydride (120 mg, 0.567 mmol) at room temperature. The resulting mixture was stirred at 50° C. overnight. The reaction was quenched with saturated NaHCO₃ (aq.) solution and extracted with DCM (2×30.0 mL). The combined organic layer was washed with brine (2×30.0 mL), dried over Na₂SO₄ and concentrated under vacuum to afford the crude residue, which was purified by silica gel column chromatography (DCM:MeOH=100:0˜90: 10 gradient elution) to give the racemate, which was separated by Prep-Chiral-HPLC with following condition:(Column: CHIRALPAK IA, 2 cm×25 cm, 5 um; Flow rate: 20 mL/min:) to afford the title compound corresponded to peak A @ 254 nm (Ret. Time: 3.925 min) (5.39 mg, 3.48%). ¹H NMR (400 MHz, DMSO) δ 12.78 (s, 1H), 10.86 (s, 1H), 8.44 (s, 1H), 8.13 (s, 1H), 7.93 (s, 1H), 7.65 (s, 1H), 7.58 (s, 1H), 7.52 (s, 1H), 7.29 (s, 1H), 7.18 (d, J=9.4 Hz, 1H), 4.20-4.17 (m, 2H), 4.14-4.10 (m, 2H), 4.05-3.98 (m, 2H), 3.74 (s, 3H), 2.88-2.81 (m, 3H), 2.64 (s, 3H), 2.57 (s, 4H), 2.37 (s, 3H), 2.25-2.20 (m, 3H), 2.10-2.05 (m, 1H), 1.99-1.91 (m, 3H), 1.55-1.42 (m, 4H), 1.24 (s, 1H), 0.82 (d, J=6.5 Hz, 4H). [M+H]⁺=818.4.

### Example 461: (R)-3-(4-(3-((4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)methyl)azetidin-1-yl)-2,6-difluorophenyl)piperidine-2,6-dione

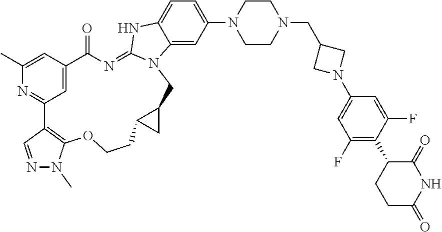 [SMILES: Cc1cc2cc(n1)-c1cnn(C)c1OCC[C@H]1C[C@@H]1CN1/C(=N/C2=O)Nc2ccc(N3CCN(CC4CN(c5cc(F)c([C@H]6CCC(=O)NC6=O)c(F)c5)C4)CC3)cc21]

The title compound was prepared in a procedure similar to that in Example 324. ¹H NMR (500 MHz, DMSO) δ_{H} 12.42 (s, 1H), 10.85 (s, 1H), 8.71 (s, 1H), 7.88 (s, 1H), 7.50 (s, 1H), 7.35 (d, J=8.7 Hz, 1H), 7.06 (s, 1H), 6.90 (dd, J=8.8, 1.9 Hz, 1H), 6.12 (d, J=11.1 Hz, 2H), 4.35-4.21 (m, 3H), 4.11 (t, J=6.7 Hz, 1H), 4.03 (dd, J=12.5, 5.0 Hz, 1H), 3.96 (t, J=7.6 Hz, 2H), 3.75 (s, 3H), 3.52 (t, J=5.9 Hz, 2H), 3.16 (s, 4H), 3.03-2.94 (m, 1H), 2.83-2.72 (m, 1H), 2.64 (d, J=7.2 Hz, 2H), 2.60-2.52 (m, 8H), 2.15-2.03 (m, 2H), 1.98-1.90 (m, 1H), 1.44 (dd, J=13.8, 5.8 Hz, 1H), 1.38-1.30 (m, 1H), 1.15 (dt, J=8.5, 4.4 Hz, 1H), 0.57-0.51 (m, 1H), 0.38 (dt, J=9.2, 4.8 Hz, 1H). [M+H]⁺=805.6.

### Example 463: 3-(5-(4-(4-((7'S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-4-methylpyridin-2-yl)piperidine-2,6-dione

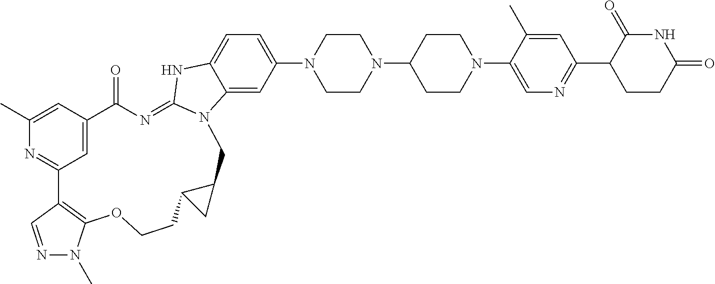 [SMILES: Cc1cc2cc(n1)-c1cnn(C)c1OCC[C@H]1C[C@@H]1CN1/C(=N/C2=O)Nc2ccc(N3CCN(C4CCN(c5cnc(C6CCC(=O)NC6=O)cc5C)CC4)CC3)cc21]

The title compound was prepared in a procedure similar to that in Example 460. ¹H NMR (500 MHz, DMSO) δ_{H} 12.44 (s, 1H), 10.80 (s, 1H), 8.71 (s, 1H), 8.15 (s, 1H), 7.88 (s, 1H), 7.50 (s, 1H), 7.35 (d, J=8.7 Hz, 1H), 7.14 (s, 1H), 7.05 (s, 1H), 6.90 (d, J=8.7 Hz, 1H), 4.36-4.21 (m, 3H), 4.11 (t, J=6.7 Hz, 1H), 3.89 (dd, J=8.7, 5.4 Hz, 1H), 3.75 (s, 3H), 3.20-3.15 (m, 6H), 2.75-2.69 (m, 6H), 2.60-2.53 (m, 5H), 2.44-2.35 (m, 1H), 2.26 (s, 3H), 2.23-2.16 (m, 1H), 2.14-2.06 (m, 2H), 1.92 (s, 2H), 1.67-1.57 (m, 2H), 1.49-1.41 (m, 1H), 1.34 (d, J=4.4 Hz, 1H), 1.15 (dd, J=8.3.4.3 Hz, 1H), 0.58-0.49 (m, 1H), 0.38 (dd, J=8.6, 4.5 Hz, 1H). [M+H]⁺=798.6.

### Example 464: (R)-3-(2,6-difluoro-4-((R)-3-((3-oxo-4-((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)piperazin-1-yl)methyl)pyrrolidin-1-yl)phenyl)piperidine-2,6-dione

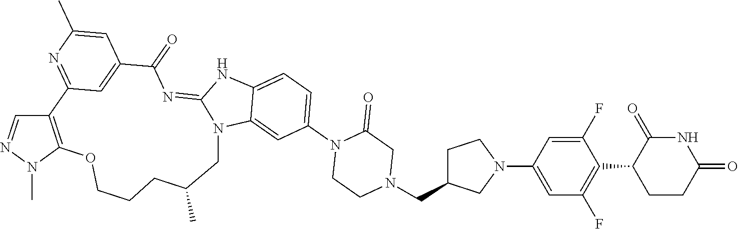 [SMILES: Cc1cc2cc(n1)-c1cnn(C)c1OCCC[C@@H](C)CN1/C(=N/C2=O)Nc2ccc(N3CCN(C[C@H]4CCN(c5cc(F)c([C@H]6CCC(=O)NC6=O)c(F)c5)C4)CC3=O)cc21]

The title compound was prepared in a procedure similar to that in Example 324. ¹H NMR (500 MHz, DMSO) δ 12.77 (s, 1H), 10.84 (s, 1H), 8.43 (s, 1H), 7.92 (s, 1H), 7.66 (s, 1H), 7.57 (s, 1H), 7.53 (d, J=8.4 Hz, 1H), 7.18 (d, J=8.3 Hz, 1H), 6.21 (d, J=12.2 Hz, 2H), 4.38-4.34 (m, 1H), 4.18 (d, J=11.6 Hz, 1H), 4.04-4.00 (m, 2H), 3.98-3.85 (m, 1H), 3.79-3.64 (m, 5H), 3.42 (t, J=8.3 Hz, 1H), 3.29 (s, 1H), 3.25-3.19 (m, 3H), 3.07-2.96 (m, 1H), 2.95-2.72 (m, 4H), 2.64-2.61 (m, 1H), 2.56 (s, 3H), 2.51 (s, 2H), 2.48 (s, 1H), 2.21 (s, 1H), 2.17-2.03 (m, 2H), 2.03-1.82 (m, 3H), 1.82-1.62 (m, 1H), 1.44 (s, 1H), 0.83 (d, J=6.4 Hz, 3H). [M+H]⁺=835.6.

### Example 465: 3-(5-(4-(4-((7¹S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-6-methylpyridin-2-yl)piperidine-2,6-dione

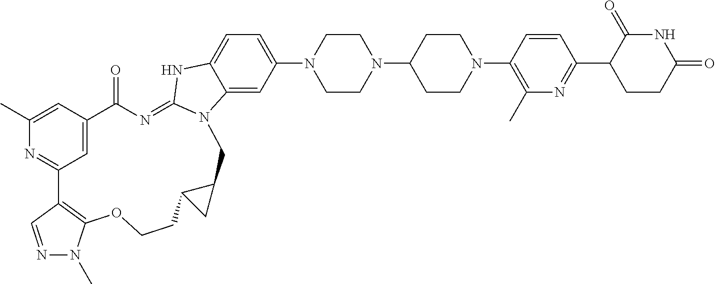 [SMILES: Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)CC3)cc21]

The title compound was prepared in a procedure similar to that in Example 460. ¹H NMR (500 MHz, DMSO) δ_{H} 12.44 (s, 1H), 10.78 (s, 1H), 8.71 (s, 1H), 7.88 (s, 1H), 7.51 (s, 1H), 7.36 (t, J=8.6 Hz, 2H), 7.10 (d, J=8.1 Hz, 1H), 7.05 (s, 1H), 6.90 (d, J=8.1 Hz, 1H), 4.37-4.22 (m, 3H), 4.11 (s, 1H), 3.89 (dd, J=9.4, 5.3 Hz, 1H), 3.75 (s, 3H), 3.20-3.12 (m, 6H), 2.72 (s, 4H), 2.65-2.54 (m, 7H), 2.42-2.36 (m, 4H), 2.24-2.16 (m, 1H), 2.09 (d, J=5.5 Hz, 2H), 1.93 (d, J=10.7 Hz, 2H), 1.63 (d, J=10.3 Hz, 2H), 1.44 (dd, J=14.3, 6.2 Hz, 1H), 1.34 (d, J=4.7 Hz, 1H), 1.15 (s, 1H), 0.57-0.50 (m, 1H), 0.38 (dd, J=8.4, 4.7 Hz, 1H). [M+H]⁺=798.6.

### Example 468: (R)-3-(4-(4-((S)-4-((7'S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)-2-(meth oxymethyl)piperazin-1-yl)piperidin-1-yl)-2,6-difluorophenyl)piperidine-2,6-dione

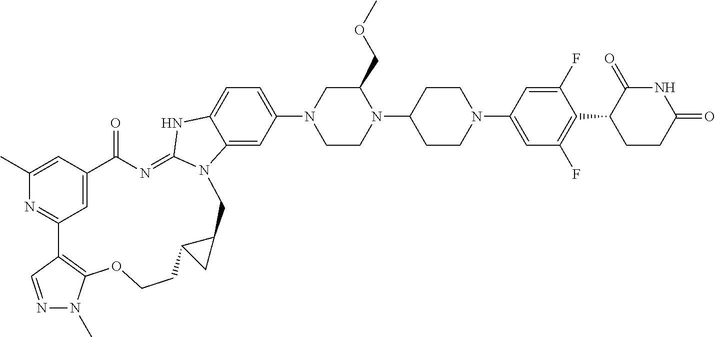 [SMILES: COC[C@@H]1CN(c2ccc3c(c2)N2C[C@H]4C[C@@H]4CCOc4c(cnn4C)-c4cc(cc(C)n4)C(=O)/N=C/2N3)CCN1C1CCN(c2cc(F)c([C@H]3CCC(=O)NC3=O)c(F)c2)CC1]

The title compound was prepared in a procedure similar to that in Example 324. ¹H NMR (500 MHz, DMSO) δ_{H} 12.44 (s, 1H), 10.87 (s, 1H), 8.71 (s, 1H), 7.88 (s, 1H), 7.50 (s, 1H), 7.35 (d, J=8.6 Hz, 1H), 7.00 (s, 1H), 6.86 (d, J=8.7 Hz, 1H), 6.64 (d, J=12.8 Hz, 2H), 4.34-4.22 (m, 3H), 4.11 (s, 1H), 4.05 (dd, J=12.5, 5.0 Hz, 1H), 3.83 (d, J=11.5 Hz, 2H), 3.75 (s, 3H), 3.51 (d, J=5.5 Hz, 2H), 3.30 (s, 3H), 3.05-2.88 (m, 6H), 2.83-2.71 (m, 6H), 2.55 (s, 3H), 2.09 (d, J=12.8 Hz, 2H), 2.00-1.93 (m, 1H), 1.88 (d, J=11.3 Hz, 1H), 1.72 (s, 1H), 1.62 (d, J=10.2 Hz, 1H), 1.45 (d, J=9.5 Hz, 2H), 1.35 (s, 1H), 1.14 (s, 1H), 0.56-0.50 (m, 1H), 0.38 (d, J=7.5 Hz, 1H). [M+H]⁺=863.6.

### Example 469: 3-(5-(4-(4-((7'S,7²R,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)pyridin-2-yl)piperidine-2,6-dione

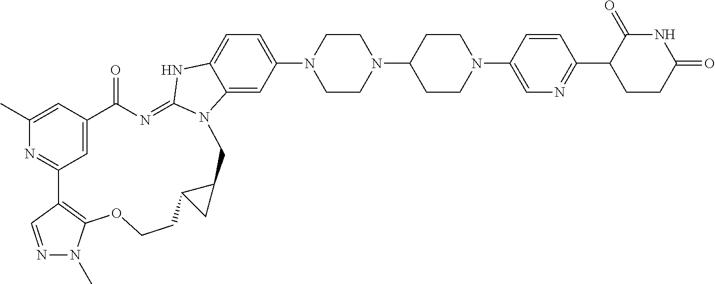 [SMILES: Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5)CC4)CC3)cc21]

The title compound was prepared in a procedure similar to that in Example 460. ¹H NMR (500 MHz, DMSO) δ_{H} 12.44 (s, 1H), 10.78 (s, 1H), 8.71 (s, 1H), 8.22 (d, J=2.8 Hz, 1H), 7.88 (s, 1H), 7.50 (s, 1H), 7.33 (dd, J=11.7, 5.8 Hz, 2H), 7.16 (d, J=8.6 Hz, 1H), 7.05 (d, J=1.4 Hz, 1H), 6.89 (dd, J=8.8, 1.8 Hz, 1H), 4.34-4.22 (m, 3H), 4.11 (s, 1H), 3.88 (dd, J=8.8, 5.3 Hz, 1H), 3.79 (d, J=12.2 Hz, 2H), 3.75 (s, 3H), 3.16 (s, 4H), 2.76-2.67 (m, 6H), 2.60-2.52 (m, 5H), 2.42 (t, J=11.0 Hz, 1H), 2.23-2.15 (m, 1H), 2.14-2.06 (m, 2H), 1.91 (d, J=11.3 Hz, 2H), 1.55 (d, J=9.0 Hz, 2H), 1.43 (dd, J=14.3, 6.3 Hz, 1H), 1.34 (dd, J=8.4, 4.4 Hz, 1H), 1.15 (dt, J=8.5, 4.3 Hz, 1H), 0.56-0.50 (m, 1H), 0.41-0.34 (m, 1H). [M+H]^{j}=784.6.

### Example 470: 3-(5-(4-(4-((7¹R,7²S,E)-1¹,2⁶-dimethyl-3-oxo-5²,5³-dihydro-1¹H,5¹H-10-oxa-4-aza-5(2,1)-benzo[d]imidazola-2(2,4)-pyridina-1(4,5)-pyrazola-7(1,2)-cyclopropanacyclodecaphane-5⁶-yl)piperazin-1-yl)piperidin-1-yl)-4-methylpyridin-2-yl)piperidine-2,6-dione

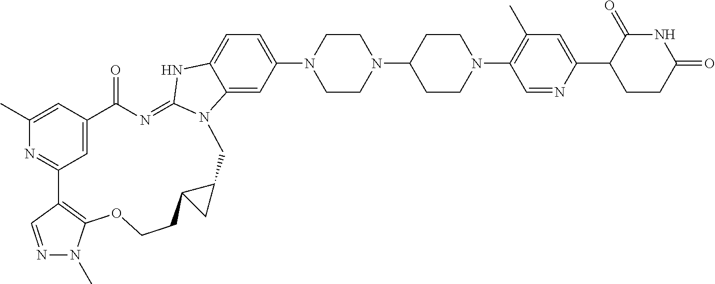 [SMILES: Cc1cc2cc(n1)-c1cnn(C)c1OCC[C@@H]1C[C@H]1CN1/C(=N/C2=O)Nc2ccc(N3CCN(C4CCN(c5cnc(C6CCC(=O)NC6=O)cc5C)CC4)CC3)cc21]

The title compound was prepared in a procedure similar to that in Example 460. ¹H NMR (500 MHz, DMSO) δ 12.45 (s, 1H), 10.80 (s, 1H), 8.71 (s, 1H), 8.16 (s, 1H), 7.88 (s, 1H), 7.51 (s, 1H), 7.36 (d, J=8.7 Hz, 1H), 7.15 (s, 1H), 7.07 (s, 1H), 6.91 (d, J=8.4 Hz, 1H), 4.38-4.21 (m, 3H), 4.12 (t, J=6.8 Hz, 1H), 3.90 (dd, J=8.7, 5.4 Hz, 1H), 3.75 (s, 3H), 3.30 (s, 3H), 3.24-3.16 (m, 5H), 2.80-2.68 (m, 5H), 2.62-2.53 (m, 6H), 2.27 (s, 2H), 2.24-2.16 (m, 1H), 2.11 (dd, J=12.4, 6.4 Hz, 2H), 1.92 (d, J=11.5 Hz, 2H), 1.63 (d, J=10.8 Hz, 2H), 1.51-1.40 (m, 1H), 1.34 (d, J=4.0 Hz, 1H), 1.15 (dd, J=8.2, 4.5 Hz, 1H), 0.58-0.50 (m, 1H), 0.39 (dd, J=8.4, 4.9 Hz, 1H). [M+H]⁺=798.7.

### Cell Line Generation

H1975-clone #28(Del19/T790M/C797S, or abbr: DTC), H1975-clone #23(Del19/C797S, or abbr: DC), H1975-clone #25(L858R/T790M/C797S, or abbr: LTC) and H1975-clone #8 (L858R/C797S, or abbr: LC). EGFR-Del19:T790M/C797S, EGFR-Del19/C797S, EGFR-L858R/T790M/C797S and EGFR-L858R/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/cell, cultured for about 2 weeks to allow single clones formation. The formed clones were screened by DNA sequencing and whole exon sequencing analysis for the desired edition. H1975-clone #28, H1975-clone #23, H1975-clone #25 and H1975-clone #8 were finally confirmed as homozygous Del19/T790M/C797S EGFR, Dell9/C797S EGFR, L858R/T790M/C797S EGFR and L858R/C797S EGFR clones, respectively.

### Cell Degradation

### Cell Treatment

On day 1, H1975-clone #28(Del19/T790M/C797S), H1975-clone #23(Del19/C797S), H1975-clone #25(L858R/T790M/C797S) and H1975-clone #8 (L858R/C797S) cells are seeded at 2000 cells/well, 30000 cells/well, 10000 cells/well or 5000 cells/well correspondingly in cell culture medium [RPMi1640(Gibco, Cat #72400-047), 10% heat-inactive FBS, 1% PS (Gibco, Cat #10378)] in Corning 96 well plate (Cat #3599).

H1975-#25, H1975-#28, H1975-#23 and H1975-#8 cells are treated with compounds diluted in 0.2% DMSO cell culture medium on day 2, incubate for 16h, 37° C., 5% CO₂. the final concentration of compounds in all assay is start with 10 uM, 5-fold dilution, total 8 doses were included.

### HTRF Assay

After 16h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group
Low control=only lysis buffer without cells, indicating that EGFR is completely degraded;
High control=Cell group with added DMSO and without compound, indicating microplate readings without EGFR degradation;
Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound: IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is: Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (H1975 # 28 DTC and H1975
# 25-LTC) result for Examples

| | |
|---|---|
| H1975 # 28 DTC | H1975 # 25-LTC |

| Example | DC₅₀(nM) | Dmax (%) | DC₅₀(nM) | Dmax (%) |
|---|---|---|---|---|
| 5 | 1.5 | 72.0 | 2 | 83 |
| 6 | 0.8 | 85.7 | 1.1 | 89.9 |
| 9 | 1.7 | 76.4 | 0.8 | 90.1 |
| 11 | 0.9 | 90.5 | 0.7 | 95.7 |
| 12 | 0.5 | 86.7 | 0.6 | 94.3 |
| 16 | | | 10.6 | 82.1 |
| 18 | 2.1 | 75.8 | 4.4 | 87.4 |
| 20 | 0.7 | 79.0 | 0.9 | 89.0 |
| 21 | | | 2.7 | 77.8 |
| 23 | 2.1 | 78.0 | 2.3 | 91.8 |
| 25 | 0.6 | 86.3 | 0.4 | 93.6 |
| 26 | 2.4 | 81.2 | 2.0 | 93.7 |
| 76 | 0.7 | 75.8 | 0.9 | 84.4 |
| 77 | 2.2 | 78.3 | 1.7 | 83.4 |
| 78 | 0.7 | 82.6 | 0.5 | 90.2 |
| 79 | 0.7 | 82.2 | 0.6 | 90.6 |
| 80 | 0.8 | 87.0 | 0.8 | 87.5 |
| 81 | 1.0 | 81.2 | 1.5 | 90.9 |
| 82 | 0.7 | 78.4 | 0.6 | 88.6 |
| 83 | 2.0 | 73.0 | 2.0 | 82.5 |
| 84 | 0.8 | 86.1 | 2.9 | 92.7 |
| 88 | | | 1.5 | 78.5 |
| 90 | | | 1.1 | 86.9 |
| 93 | | | 1.9 | 92.7 |
| 94 | 0.7 | 80.5 | 0.5 | 90.6 |
| 96 | 0.4 | 84.3 | 0.6 | 87.8 |
| 99 | 0.7 | 75.6 | 1.4 | 82.7 |
| 100 | 0.7 | 72.8 | 1.0 | 85.5 |
| 106 | 1.2 | 74.4 | 1.7 | 79.1 |
| 107 | | | 0.6 | 82.5 |
| 59 | 1.48 | 78.58 | 2.92 | 81.96 |
| 64 | 1.51 | 80.65 | 3.13 | 76.83 |
| 144 | 0.508 | 55.85 | 0.761 | 80.82 |
| 147 | 2.41 | 69.47 | 1.83 | 81.39 |
| 148 | 3.09 | 70.93 | 1.89 | 78.71 |
| 150 | 2.17 | 79.66 | 0.705 | 87.94 |
| 154 | 0.994 | 78.94 | 0.998 | 79.43 |
| 156 | 1.2 | 72.74 | 0.634 | 86.86 |
| 159 | 1.77 | 79.77 | 1.48 | 79.92 |
| 158 | 0.868 | 72.14 | 0.852 | 82.21 |
| 162 | 0.557 | 71.45 | 0.598 | 79.08 |
| 163 | 1.76 | 66.41 | 1.14 | 83.06 |
| 164 | 1.95 | 73.21 | 1.46 | 83.13 |
| 165 | 2.95 | 74.73 | 3.1 | 79.87 |
| 172 | 0.663 | 75.08 | 0.954 | 90.56 |
| 185 | 0.752 | 60.18 | 1.04 | 87.14 |
| 187 | 1.8 | 80.62 | 1.01 | 86.08 |
| 190 | 0.742 | 64.16 | 1.17 | 82.21 |
| 194 | 0.836 | 79.44 | 1.18 | 84.44 |
| 195 | 0.712 | 76.37 | 0.731 | 85.46 |
| 196 | 0.598 | 78.44 | 0.716 | 87.09 |
| 199 | 0.752 | 73.3 | 0.638 | 76.46 |
| 200 | 1.38 | 73.04 | 1.22 | 86.01 |
| 202 | 1.32 | 72.07 | 0.89 | 78.84 |
| 256 | 0.608 | 83.08 | 0.326 | 88.14 |
| 211 | 0.901 | 75.24 | 0.852 | 85.07 |
| 214 | 1.32 | 72.32 | 2.59 | 79.37 |
| 216 | 3.02 | 82.64 | 9.55 | 81.83 |
| 221 | 0.901 | 85.41 | 0.705 | 94.05 |
| 229 | 1.79 | 78.12 | 1.27 | 86.88 |
| 232 | 0.292 | 82.7 | 0.318 | 92.03 |
| 233 | 0.806 | 86.73 | 1.05 | 91.18 |
| 234 | 0.637 | 85.75 | 1.09 | 90.1 |
| 235 | 0.52 | 87.23 | 0.824 | 88.72 |
| 236 | 0.42 | 87.56 | 0.546 | 88.39 |
| 238 | 0.565 | 83.62 | 0.602 | 93.22 |
| 239 | 0.205 | 83.87 | 0.604 | 87.89 |
| 241 | 0.328 | 76.68 | 0.403 | 87.65 |
| 242 | 0.352 | 88.18 | 0.321 | 93.2 |
| 243 | 2.9 | 86.39 | 0.677 | 92.13 |
| 244 | 0.548 | 78.89 | 0.167 | 95.29 |
| 245 | 0.534 | 81.9 | 0.487 | 94.03 |
| 246 | 0.602 | 85.73 | 0.613 | 93.75 |
| 247 | 0.611 | 87.12 | 0.6 | 92.48 |
| 250 | 0.59 | 82.13 | 1.06 | 88.94 |
| 251 | 0.611 | 80.86 | 0.888 | 85.48 |
| 252 | 0.606 | 77.32 | 0.584 | 92.38 |
| 253 | 0.59 | 82.5 | 0.289 | 93.8 |
| 254 | 0.624 | 86.3 | 0.91 | 91.22 |
| 255 | 0.566 | 81.82 | 0.599 | 91.09 |
| 257 | 0.57 | 80.82 | 0.512 | 87.99 |
| 258 | 0.577 | 81.96 | 0.381 | 88.17 |
| 259 | 0.725 | 83.21 | 0.841 | 89.18 |
| 260 | 0.633 | 86.51 | 0.544 | 93.98 |
| 261 | 0.621 | 83.61 | 0.51 | 91.77 |
| 262 | 0.474 | 76.44 | 0.543 | 84.2 |
| 263 | 0.597 | 78.67 | 0.351 | 93.63 |
| 264 | 0.677 | 68.8 | 0.546 | 89.85 |
| 265 | 0.625 | 79.26 | 0.246 | 90.9 |
| 266 | 0.65 | 75.93 | 1.51 | 81.37 |
| 267 | 0.624 | 76.64 | 0.602 | 91.66 |
| 268 | 0.439 | 81.98 | 0.53 | 89.84 |
| 269 | 0.638 | 82.6 | 0.316 | 92.67 |
| 270 | 0.614 | 81.16 | 0.429 | 92.49 |
| 271 | 1.15 | 70.36 | 0.557 | 81.21 |
| 272 | 1.52 | 76.03 | 1.36 | 87.25 |
| 273 | 0.489 | 66.66 | 4.58 | 88.78 |
| 274 | 0.691 | 48.56 | 1.63 | 69.25 |
| 275 | 0.798 | 78.92 | 1.01 | 88.86 |
| 276 | 1.74 | 71.19 | 0.592 | 88.03 |
| 278 | 0.567 | 52.21 | 0.804 | 74.15 |
| 279 | 0.75 | 82.08 | 1.33 | 83.0 |
| 280 | 0.657 | 77.24 | 0.44 | 88.49 |
| 281 | 0.501 | 75.01 | 0.411 | 90.37 |
| 282 | 0.657 | 77.76 | 0.752 | 82.94 |
| 283 | 0.617 | 72.52 | 0.494 | 80.22 |
| 285 | 0.667 | 77.98 | 0.651 | 89.81 |
| 286 | 0.652 | 72.5 | 0.45 | 91.19 |
| 287 | 0.918 | 88.53 | 0.581 | 93.48 |
| 288 | 0.488 | 79.62 | 0.662 | 92.83 |
| 289 | 0.624 | 91.07 | 0.454 | 93.78 |
| 290 | 0.64 | 80.73 | 0.644 | 89.13 |
| 291 | 0.513 | 72.04 | 0.63 | 78.99 |
| 292 | 0.608 | 85.48 | 0.514 | 89.4 |
| 293 | 0.997 | 75.49 | 1.56 | 86.33 |
| 295 | 0.313 | 78.39 | 0.478 | 89.48 |
| 296 | 0.842 | 51.36 | 0.707 | 84.8 |
| 297 | 0.292 | 79.64 | 0.432 | 91.74 |
| 298 | 1.2 | 66.58 | 1.14 | 90.07 |
| 299 | 0.611 | 66.17 | 0.468 | 86.89 |
| 300 | 1.48 | 73.74 | 0.653 | 88.24 |
| 303 | 0.246 | 63.64 | 0.582 | 80.86 |
| 304 | 0.595 | 78.73 | 0.61 | 79.9 |
| 305 | 0.668 | 80.65 | 0.861 | 90.59 |
| 306 | 0.614 | 63.09 | 0.742 | 84.31 |
| 307 | 1.28 | 52.25 | 1.78 | 84.77 |
| 308 | 0.646 | 64.88 | 0.702 | 88.53 |
| 309 | 0.684 | 65.2 | 0.459 | 82.96 |
| 310 | 0.578 | 80.51 | 0.464 | 87.85 |
| 312 | 0.689 | 47.95 | 0.906 | 85.79 |
| 313 | 0.967 | 70.56 | 1.25 | 81.01 |
| 314 | 3.37 | 50.86 | 2.59 | 75.32 |
| 315 | 3.18 | 63.86 | 1.37 | 80.52 |
| 316 | 0.925 | 47.67 | 0.655 | 77.86 |
| 317 | 0.728 | 52.86 | 1.14 | 80.16 |
| 318 | 0.995 | 67.67 | 1.33 | 76.37 |
| 319 | 0.643 | 74.25 | 1.26 | 83.41 |
| 320 | 0.674 | 71.04 | 0.591 | 86.2 |
| 321 | 1.18 | 73.52 | 1.17 | 88.25 |
| 322 | 0.934 | 74.99 | 1.21 | 86.8 |
| 323 | 1.03 | 49.97 | 1.03 | 63.85 |
| 324 | 0.859 | 78.7 | 1.02 | 91.08 |
| 326 | 1.71 | 81.61 | 1.88 | 88.86 |
| 327 | 1.09 | 74.51 | 3.17 | 83.67 |
| 329 | 2.19 | 68.61 | 2.97 | 78.67 |
| 330 | 0.633 | 66.1 | 0.972 | 87.07 |
| 331 | 1.33 | 73.69 | 0.71 | 97.08 |
| 332 | 1.31 | 86.39 | 0.995 | 91.16 |
| 333 | 0.684 | 75.17 | 0.687 | 86.11 |
| 335 | 0.789 | 60.16 | 0.657 | 79.36 |
| 336 | 0.304 | 72.28 | 0.386 | 88.43 |
| 337 | 0.608 | 79.69 | 0.535 | 92.0 |
| 339 | 0.671 | 70.76 | 0.865 | 80.34 |
| 340 | 0.587 | 83.73 | 0.28 | 91.69 |
| 341 | 0.621 | 79.84 | 0.54 | 86.83 |
| 342 | 0.607 | 77.52 | 0.62 | 90.82 |
| 343 | 0.609 | 76.11 | 0.599 | 90.48 |
| 344 | 0.62 | 83.22 | 0.386 | 92.8 |
| 345 | 0.662 | 74.46 | 0.634 | 92.48 |
| 346 | 0.411 | 78.78 | 0.526 | 84.85 |
| 345 | 0.7 | 74.5 | 0.6 | 92.5 |
| 350 | 0.662 | 86.74 | 0.614 | 90.36 |
| 351 | 0.561 | 83.91 | 0.614 | 91.68 |
| 352 | 0.814 | 79.43 | 0.847 | 85.61 |
| 354 | 0.501 | 72.74 | 0.590 | 89.34 |
| 357 | 0.903 | 73.73 | 0.824 | 87.61 |
| 358 | 1.300 | 75.67 | 0.622 | 89.62 |
| 359 | 0.751 | 83.57 | 0.841 | 87.08 |
| 363 | 0.614 | 78.54 | 0.643 | 87.45 |
| 364 | 0.633 | 75.27 | 1.117 | 86.44 |
| 365 | 0.625 | 80.74 | 0.878 | 91.11 |
| 366 | 0.539 | 84.74 | 0.372 | 91.83 |
| 367 | 0.623 | 80.77 | 1.306 | 87.75 |
| 368 | 0.623 | 80.06 | 0.546 | 88.05 |
| 373 | 0.656 | 69.22 | 0.470 | 84.92 |
| 374 | 0.583 | 81.48 | 0.591 | 92.72 |
| 379 | 0.682 | 67.89 | 0.503 | 80.86 |
| 380 | 2.245 | 76.83 | 3.349 | 80.23 |
| 381 | 1.058 | 60.14 | 0.900 | 83.62 |
| 382 | 0.617 | 86.76 | 0.233 | 87.55 |
| 383 | 2.193 | 73.18 | 1.344 | 88.78 |
| 384 | 1.504 | 75.83 | 1.668 | 87.65 |
| 385 | 1.576 | 63.01 | 2.632 | 82.27 |
| 386 | 2.014 | 72.30 | 2.700 | 86.62 |
| 387 | 0.782 | 76.00 | 1.462 | 87.19 |
| 388 | 0.776 | 80.94 | 1.111 | 86.55 |
| 390 | 1.686 | 73.87 | 2.491 | 87.66 |
| 391 | 1.544 | 85.34 | 1.558 | 87.55 |
| 392 | 0.821 | 75.48 | 1.289 | 86.67 |
| 393 | 0.540 | 77.73 | 0.340 | 89.77 |
| 394 | 0.901 | 69.35 | 0.787 | 74.14 |
| 395 | 0.526 | 77.96 | 1.022 | 84.54 |
| 396 | 0.681 | 63.49 | 0.622 | 83.60 |
| 397 | 2.134 | 76.68 | 2.010 | 88.31 |
| 398 | 4.504 | 72.53 | 4.701 | 90.32 |
| 400 | 0.628 | 79.74 | 0.915 | 89.78 |
| 401 | 3.182 | 70.19 | 1.374 | 81.14 |
| 402 | 0.525 | 82.56 | 0.633 | 88.27 |
| 403 | 0.857 | 82.98 | 1.069 | 87.50 |
| 404 | 1.687 | 83.57 | 0.940 | 88.50 |
| 405 | 1.265 | 78.67 | 0.677 | 80.49 |
| 406 | 1.092 | 85.15 | 1.645 | 89.59 |
| 407 | 2.086 | 78.49 | 2.287 | 87.89 |
| 418 | 3.200 | 91.56 | 0.517 | 60.66 |
| 421 | 0.639 | 84.08 | 0.604 | 87.50 |
| 422 | 0.652 | 83.55 | 0.856 | 92.17 |
| 423 | 0.655 | 67.20 | 1.405 | 78.75 |
| 424 | 0.794 | 82.75 | 2.510 | 89.82 |
| 425 | 0.957 | 83.85 | 0.730 | 92.69 |
| 437 | 0.534 | 88.76 | 1.010 | 91.04 |
| 438 | 0.983 | 72.51 | 0.964 | 85.62 |
| 439 | 1.519 | 76.03 | 1.40 | 87.30 |
| 440 | 0.884 | 85.39 | 0.936 | 93.49 |
| 441 | 0.643 | 89.27 | 0.857 | 89.19 |
| 442 | 0.721 | 70.20 | 0.742 | 84.41 |
| 443 | 0.633 | 78.26 | 1.250 | 86.56 |
| 444 | 1.300 | 82.11 | 2.100 | 88.95 |
| 445 | 2.140 | 74.88 | 0.559 | 84.48 |
| 446 | 1.190 | 76.42 | 0.696 | 91.81 |
| 447 | 0.645 | 76.31 | 0.652 | 84.23 |
| 448 | 0.584 | 88.36 | 0.237 | 92.27 |
| 449 | 1.010 | 72.50 | 1.280 | 86.16 |
| 450 | 0.647 | 83.01 | 0.678 | 88.65 |
| 451 | 0.606 | 81.53 | 0.881 | 90.93 |
| 452 | 1.290 | 75.73 | 0.980 | 78.48 |
| 453 | 1.430 | 82.83 | 3.170 | 85.35 |
| 454 | 0.765 | 68.28 | 1.080 | 85.98 |
| 455 | 0.912 | 85.22 | 0.537 | 90.8 |
| 456 | 0.542 | 84.5 | 0.676 | 93.18 |
| 457 | 0.709 | 91.81 | 0.632 | 92.84 |
| 458 | 0.985 | 92.47 | 0.809 | 92.65 |
| 459 | 0.624 | 73.95 | 0.431 | 84.07 |
| 460 | 0.629 | 75.27 | 0.431 | 89.59 |
| 461 | 0.702 | 84.7 | 1.64 | 90.63 |
| 462 | 1.1 | 78.96 | 1.38 | 90.33 |
| 463 | 0.604 | 90.22 | 0.624 | 91.81 |
| 464 | 0.678 | 82.71 | 0.646 | 84.98 |
| 465 | 0.319 | 90.39 | 0.472 | 94.82 |
| 468 | 2.25 | 85.54 | 2.12 | 88.11 |
| 469 | 0.594 | 76.63 | 0.794 | 82.67 |
| 470 | 0.617 | 86.76 | 0.849 | 92.08 |

TABLE 3
Degradation (H1975 # 23 DC and H1975 #
8-LC) result for Example 5 to Example 9

| | |
|---|---|
| H1975 #23 DC | H1975 #8 LC |

| Example | DC₅₀(nM) | Dmax (%) | DC₅₀(nM) | Dmax |
|---|---|---|---|---|
| 5 | 0.6 | 67.6 | | |
| 6 | 1.7 | 78.2 | 3.0 | 63.1 |
| 9 | 1.0 | 82.6 | 3.2 | 64.6 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 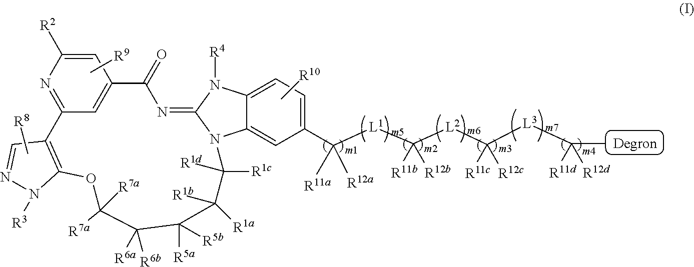
or a N-oxide thereof, or a pharmaceutically acceptable salt thereof, or a stereoisomer thereof, or a deuterated analog thereof, or a prodrug thereof,
wherein:
R³ and R⁴ are each independently absence, hydrogen, —C₁₋₈alkyl, —C₂₋₈alkenyl, —C₂₋₈alkynyl or —C₃₋₈cycloalkyl; each said —C₁₋₈alkyl, —C₂₋₈alkenyl, —C₂₋₈alkynyl or —C₃₋₈cycloalkyl is optionally substituted with at least one substituent selected from hydrogen, halogen, —C₁₋₈alkoxy, —C₃₋₈cycloalkyl or —CN;
R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or 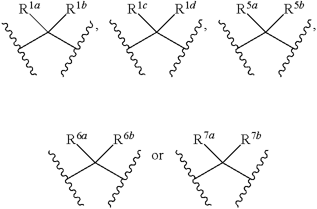
can be optionally replaced with O or NR^{a};
two geminal or adjacent substituent from R^{1a}, R^{1b}, R^{1c}, R^{1d}, R^{5a}, R^{5b}, R^{6a}, R^{6b}, R^{7a} and R^{7b} with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
R^{11a}, R^{11b}, R^{11c}, R^{11d}, R^{12a}, R^{12b}, R^{12c} and R^{12d} are each independently absence, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN; or
R^{11a} and R^{12a} together with the carbon atoms to which they are attached, form a 3- to 12-membered ring, said ring comprising 0-3 heteroatoms independently selected from nitrogen, oxygen or sulfur, said ring is optionally substituted with at least one substituent selected from hydrogen, halogen, —C₁₋₈alkyl, —C₂₋₈alkenyl, —C₂₋₈alkynyl, —C₁₋₈alkoxy or —CN:
R^{11b} and R^{12b} together with the carbon atoms to which they are attached, form a 3- to 12-membered ring, said ring comprising 0-3 heteroatoms independently selected from nitrogen, oxygen or sulfur;
said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
R^{11a} and R^{12c} together with the carbon atoms to which they are attached, form a 3- to 12-membered ring, said ring comprising 0-3 heteroatoms independently selected from nitrogen, oxygen or sulfur, said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
R^{11d} and R^{12d} together with the carbon atoms to which they are attached, form a 3- to 12-membered ring, said ring comprising 0-3 heteroatoms independently selected from nitrogen, oxygen or sulfur;
said ring is optionally substituted with at least one substituent selected from hydrogen, halogen, —C₁₋₈alkyl, —C₂₋₈alkenyl, —C₂₋₈alkynyl, —C₁₋₈alkoxy or —CN;
L¹ is independently selected from —O—, —NR^{a}—, —C(O)—, *^{L1}—C(O)NR—**^{L1}, *^{L1}—C(O)O—**^{L1}, *^{L1}—NR^{a}C(O)—**^{L1}, *^{L1}—OC(O)—**^{L1}, 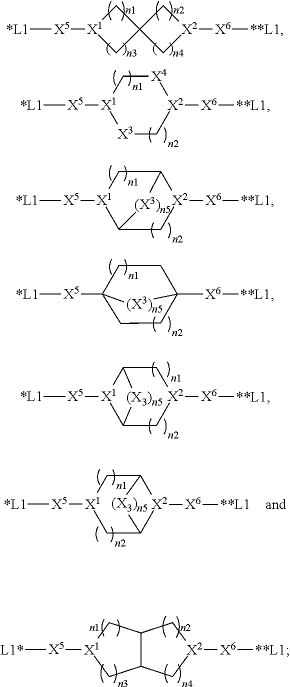
wherein each of said 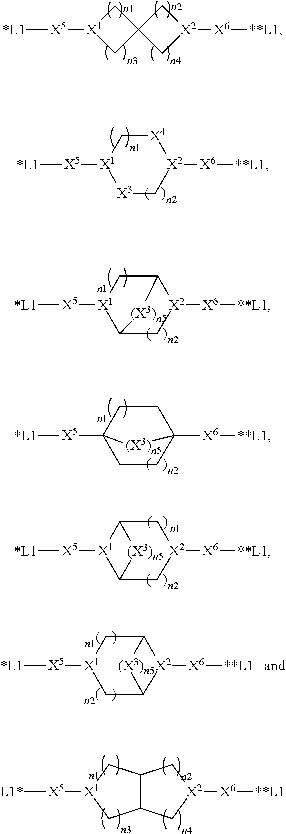
is optionally substituted with at least one R^{L1c};
wherein *^{L1} refers to the position attached to the 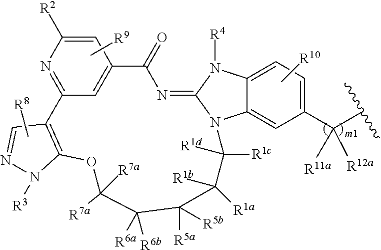
moiety, and **^{L1} refers to the position attached to the 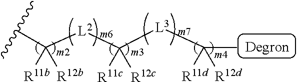
moiety;
L² is independently selected from —O—, —NR*—, —C(O)—, *^{L2}—C(O)NR—**^{L2}*^{L2}—C(O)O—**^{L2},*^{L2}—NR^{a}C(O)—**^{L2}, *^{L2}—OC(O)—**^{L2}, 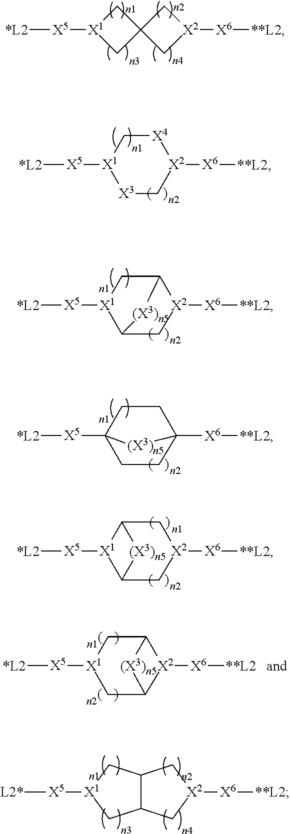
wherein each of said 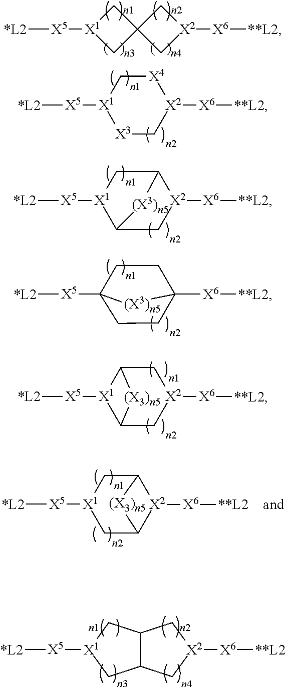
is optionally substituted with at least one R^{L2c};
wherein *^{L2} refers to the position attached to the 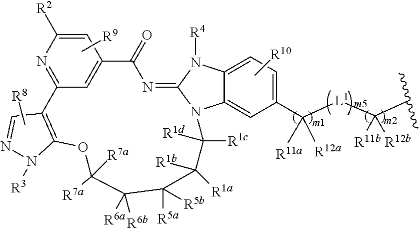
moiety,
and **^{L2} refers to the position attached to the 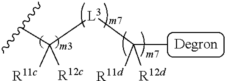
moiety:
L³ is independently selected from —O—, —NR^{a}—, —C(O)—, *^{L3}—C(O)NR^{a}—**^{L3}, *^{L3}—C(O)O—**^{L3}, *^{L3}—NR^{a}C(O)—**^{L3},*^{L3}—OC(O)—**^{L3}, 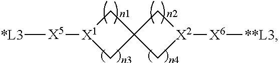
wherein each of said 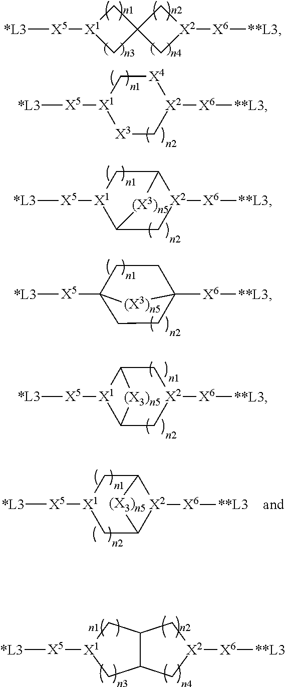
is optionally substituted with at least one R^{L3c};
wherein *^{L3} refers to the position attached to the 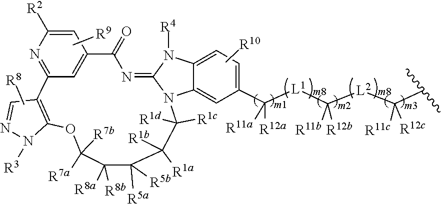
moiety, and **^{L3} refers to the position attached to the 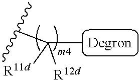
moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca};
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl; 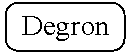
is selected from 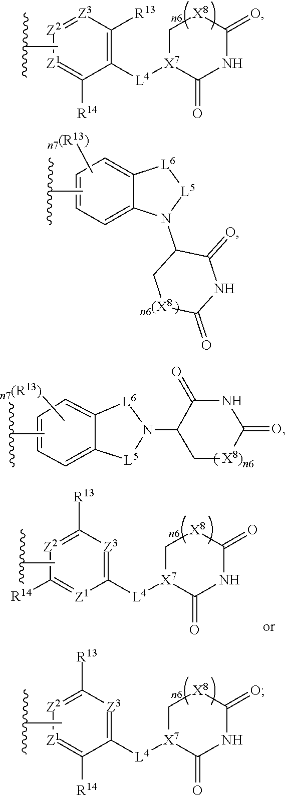
Z¹, Z² and Z³ are each independently N or CR^{z}, provided that Z¹, Z² and Z³ are not N at the same time;
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Ze};
the 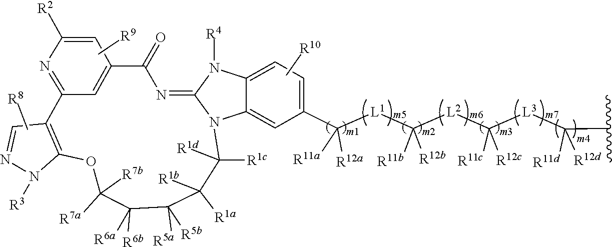
moiety is linked to the 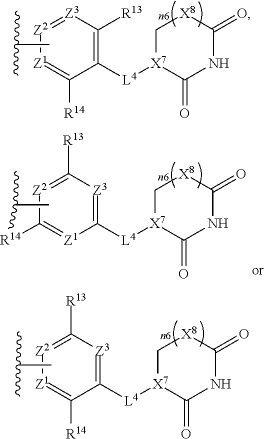
moiety via any one of Z¹, Z² or Z³ which is CR^{z} and R^{z} is absence;
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}O₂R^{13b}, or —NR^{13a}—SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{1c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl:
L⁴, L⁵ and L⁶ are each independently selected from a absence, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{z}, or N:
at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}- and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl:
m1, m2, m3 and m4 are each independently 0, 1 or 2; provided that m1+m2+m3+m4≤4;
m5, m6 and m7 are each independently 0 or 1; provided that m5+m6+m7≥1;
n1, n2, n3, n4 and n5 are each independently 0, 1, 2 or 3;
n6, n7 and n8 are each independently 0, 1, 2, 3 or 4;
provided that:
for any one of L¹, L² or L³, when X¹ is N, X¹ is single bond, absence, —C(O)—; and/or when X² is N, X⁶ is single bond, absence, —C(O)—;
when L¹ is 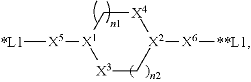
when X¹ is N, X⁵ is single bond, absence, —C(O)-; and/or X³ is —CR^{a}R^{b}— when X² is N, X⁶ is single bond, absence, —C(O)—, and/or X⁴ is —CR^{a}R^{b}—;
when L² is 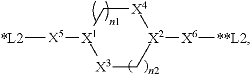
when X¹ is N, X⁵ is single bond, absence, —C(O)—; and/or X³ is —CR^{a}R^{b}— when X² is N, X⁶ is single bond, absence, —C(O)—, and/or X⁴ is —CR^{a}R^{b}—;
when L³ is 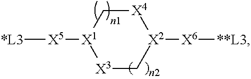
when X¹ is N, X⁵ is single bond, absence, —C(O)—; and/or X³ is —CR^{a}R^{b}— when X² is N, X⁶ is single bond, absence, —C(O)—, and/or X⁴ is —CR^{a}R^{b}—.

2. The compound of claim 1, wherein the compound is selected from formula (II) 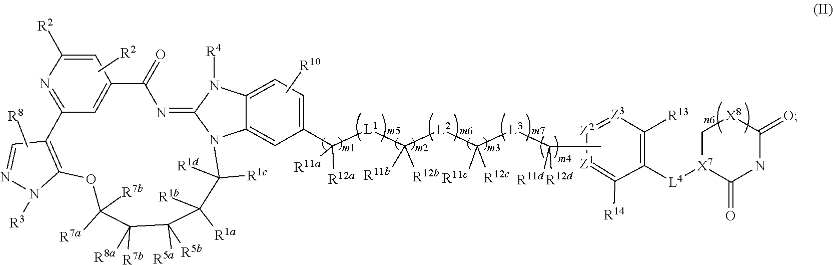
wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, L⁴, Z¹, Z², Z³, X⁷, X⁸, m1, m2, m3, m4, m5, m6, m7 and n6 are defined as claim 1.

3. The compound of any one of the preceding claims, wherein the compound is selected from formula (III) 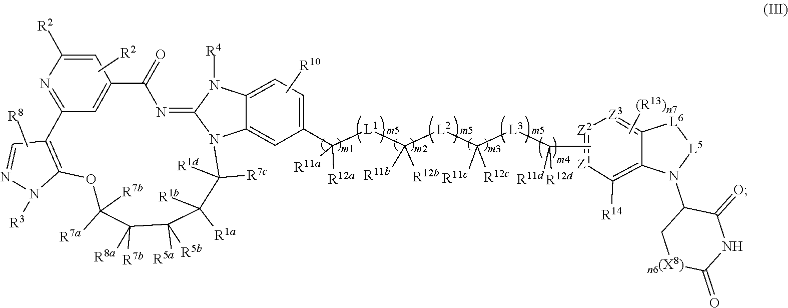
wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, L¹, L², L³, L⁵, L⁶, X¹, m1, m2, m3, m4, m5, m6, m7, n6 and n7 are as defined in any one of the preceding claims.

4. The compound of any one of the preceding claims, wherein the compound is selected from formula (IV) 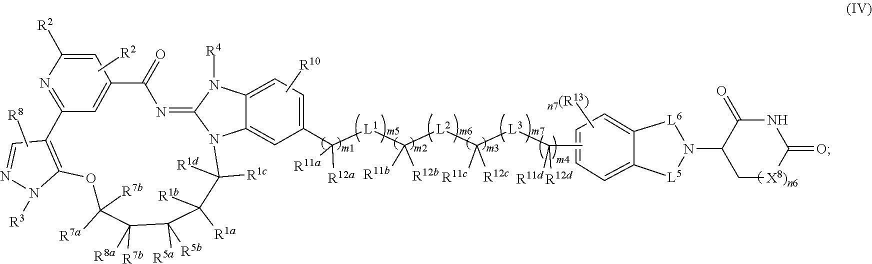
wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, L¹, L², L³, L⁵, L⁶, X⁸, m1, m2, m3, m4, m5, m6, m7, n6 and n7 are as defined in any one of the preceding claims.

5. The compound of any one of the preceding claims, wherein the compound is selected from formula (Va), (Vb), (Vc) or (Vd), 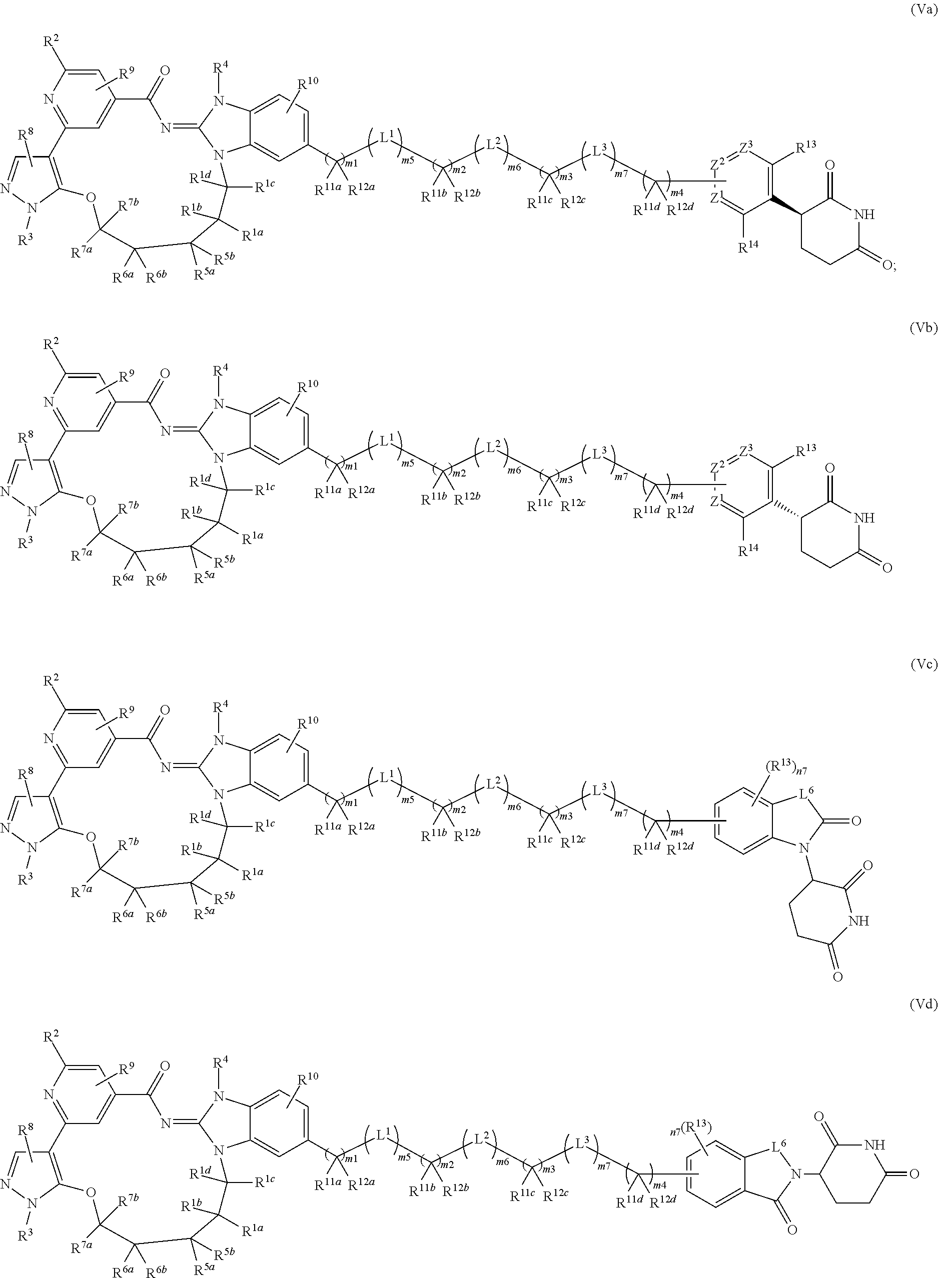
wherein, R^{1a}, R^{1b}, R^{1c}, R^{1d}, R², R³, R⁴, R^{5a}, R^{5b}, R^{6a}, R^{6b}, R^{7a}, R^{7b}, R², R⁹, R¹⁰, R^{11a}, R^{11b}, R^{11c}, R^{11d}, R^{12a}, R^{12b}, R^{12c}, R^{12d}, R¹³, R¹⁴, L¹, L², L³, L⁶, Z¹, Z², Z³, m1, m2, m3, m4, m5, m6, m7 and n7 are as defined in any one of the preceding claims.

6. The compound of any one of the preceding claims, wherein the compound is selected from formula (VIa), (VIb), (VIc), (VId) or (VIe), 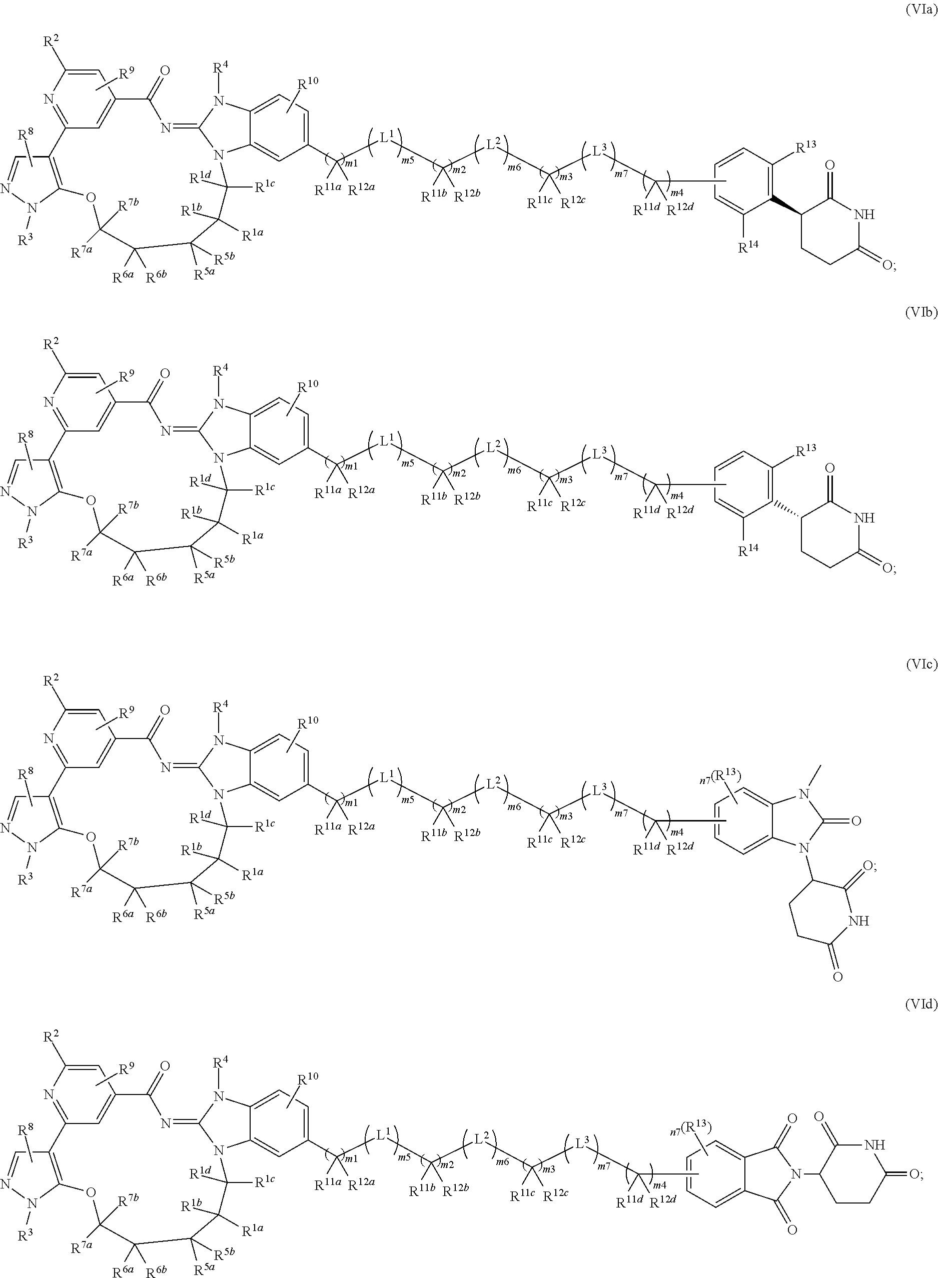 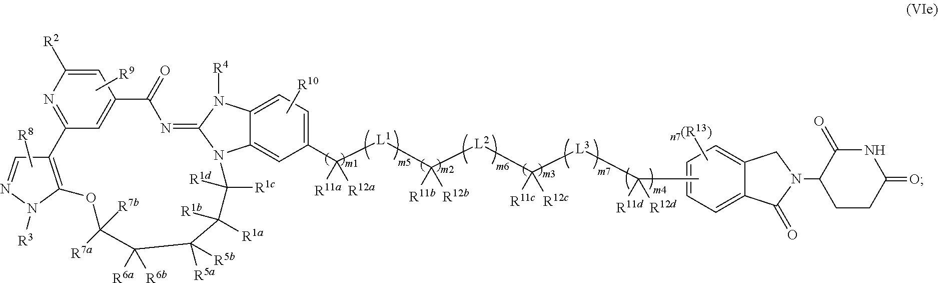
wherein, R^{1a}, R^{1b}, R^{1c}, R², R³, R⁴, R^{5a}, R^{5b}, R^{6a}, R^{6b}, R^{7a}, R^{7b}, R⁸, R⁹, R¹⁰, R^{11a}, R^{11b}, R^{11c}, R^{11d}, R^{12a}, R^{12b}, R^{12c}, R^{12d}, R¹³, R¹⁴, L¹, L², L³, m1, m2, m3, m4, m5, m6, m7 and n7 are as defined in any one of the preceding claims.

7. The compound of any one of the preceding claims, wherein the compound is selected from formula (VIIa), (VIIb), (VIIc), (VIId) or (VIIe), 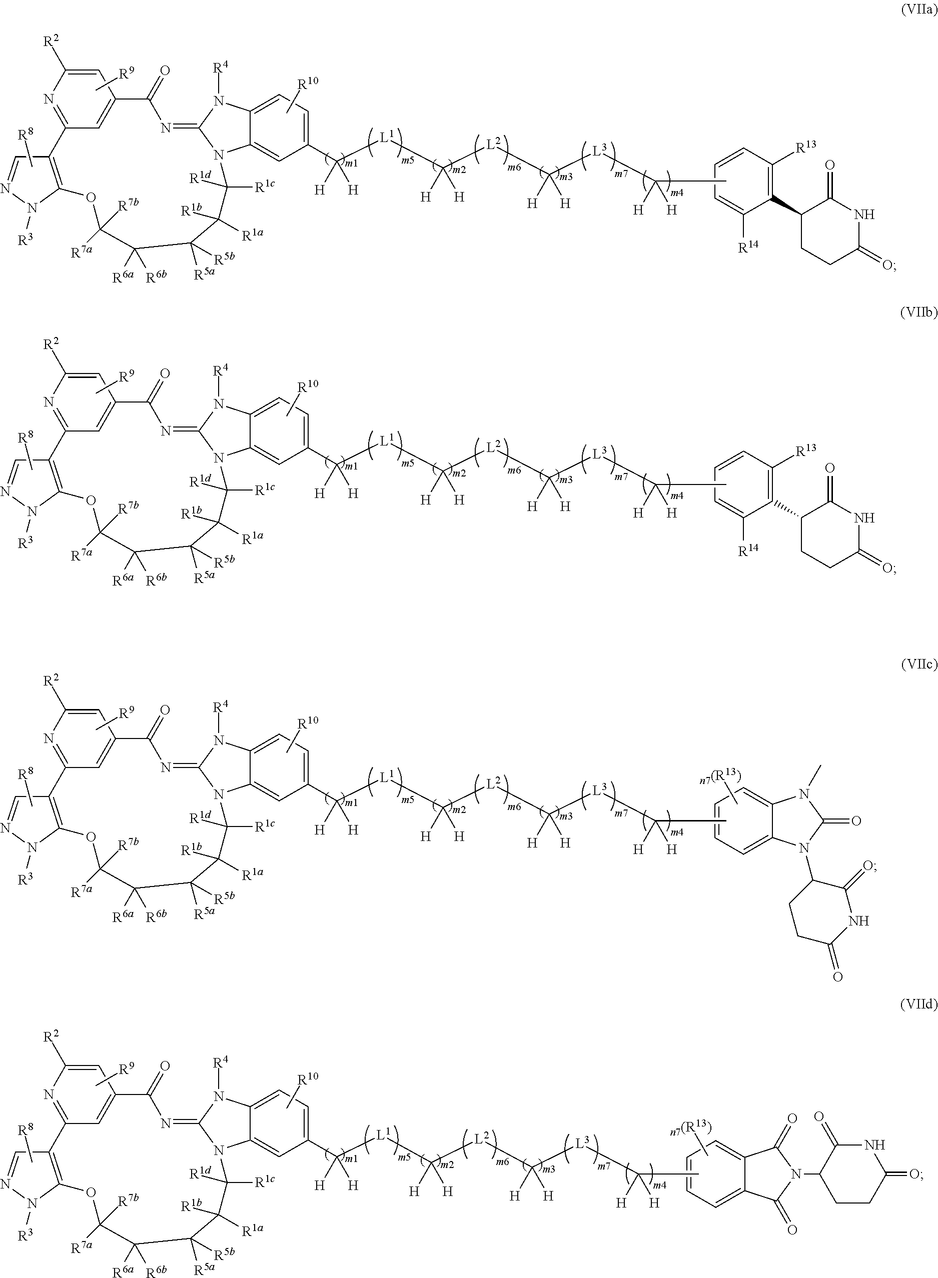 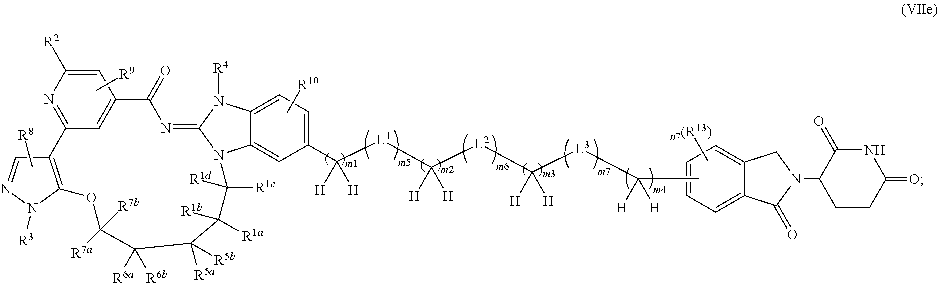
wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R¹³, R¹⁴, L¹, L², L³, m1, m2, m3, m4, m5, m6, m7 and n7 are as defined in any one of the preceding claims.

8. The compound of any one of the preceding claims, wherein the compound is selected from formula (VIIIa), (VIIIb), (VIIIc) or (VIIId), 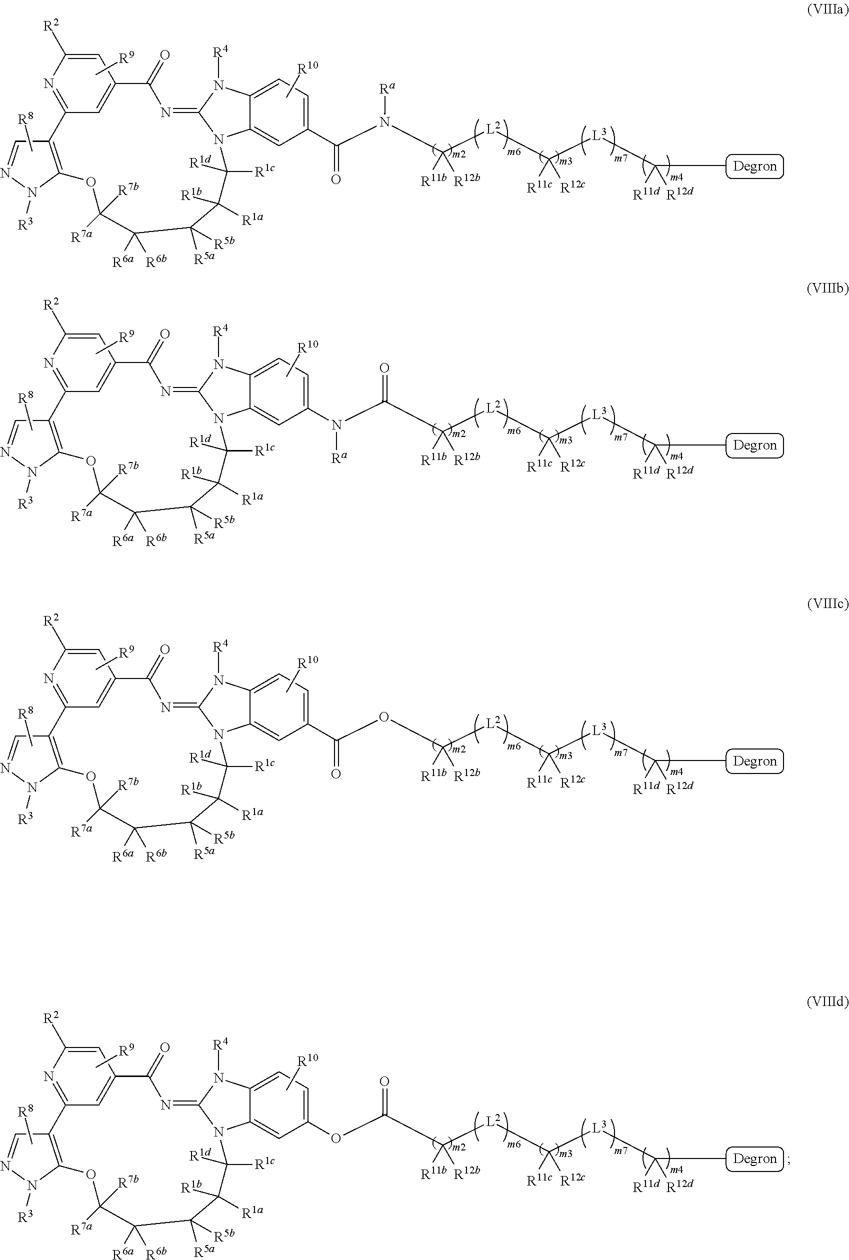
wherein, R^{1a}, R^{1b}, R^{1c}, R^{1d}, R², R³, R⁴, R^{5a}, R^{5b}, R^{6a}, R^{6b}, R^{7a}, R^{7b}, R⁸, R⁹, R¹⁰, R^{11b}, R^{11c}, R^{11d}, R^{12b}, R^{12c}, R^{12d}, R¹³, R¹⁴, L², L³, m2, m3, m4, m6, m7 and Degron are as defined in any one of the preceding claims.

9. The compound of any one of the preceding claims, wherein m1+m2+m3+m4≤3.

10. The compound of any one of the preceding claims, wherein m1+m2+m3+m4=1, 2 or 3; preferably, m1+m2+m3+m4=1 or 2.

11. The compound of any one of the preceding claims, wherein the number of —CH₂— in 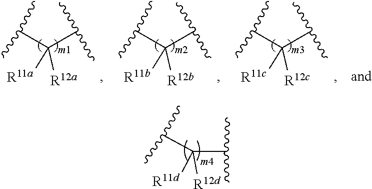
moieties is no more than 4, preferably is no more than 3, even more preferably is no more than 2.

12. The compound of any one of the preceding claims, wherein R³ and R⁴ are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl or cyclooctyl; said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl or cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN;

13. The compound of any one of the preceding claims, wherein R³ and R⁴ are each independently hydrogen, methyl, ethyl, propyl, butyl, pentyl, hexyl, heptyl, octyl, cyclopropyl, cyclobutyl, cyclopentyl, cyclohexyl, cycloheptyl or cyclooctyl: preferably R³ is independently methyl or cyclopropyl, and R⁴ is hydrogen.

14. The compound of any one of the preceding claims, wherein R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN; wherein each said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl and cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

15. The compound of any one of the preceding claims, wherein R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₃: preferably R^{1a}, R^{1b}, R^{1c}, R^{1d}, R^{5a}, R^{5b}, R^{6a}, R^{6b}, R^{7a}, R^{7b}, R², R⁸, R⁹ and R¹⁰ are each independently hydrogen, F, Cl, methyl, methoxy, cyclopropyl, —CF₃ or —CHF₂, —CH₂OCH₃: more preferably, R² is hydrogen, methyl, methoxy, cyclopropyl or —CF₃; R⁸ is hydrogen, F, methyl, —CF₃ or —CHF₂; R⁹ is hydrogen or F; R¹⁰ is hydrogen, F, Cl, methyl or —CH₂OCH₃, R^{1a}, R^{1b}, R^{1c}, R^{1d}, R^{5a}, R^{5b}, R^{6a}, R^{6b}, R^{7a} and R^{7b} are each independently hydrogen, F or methyl.

16. The compound of any one of the preceding claims, wherein (R^{1a} and R^{1b}), (R^{1c} and R^{1d}), (R^{5a} and R^{5b}), (R^{6a} and R^{6b}), (R^{7a} and R^{7b}), (R^{1a} and R^{1c}), (R^{1a} and R^{1d}), (R^{1b} and R^{1c}), (R^{1b} and R^{1d}), (R^{1a} and R^{5a}), (R^{1a} and R^{5b}), (R^{1b} and R^{5a}), (R^{1b} and R^{5b}), (R^{5a} and R^{6a}), (R^{5a} and R^{6b}), (R^{5b} and R^{6a}), (R^{5b} and R^{6b}), (R^{6a} and R^{7a}), (R^{6a} and R^{7b}), (R^{6b} and R^{7a}) or (R^{6b} and R^{7b}) with the carbon atoms to which they are attached, form a 3-, 4-, 5-, 6-, 7- or 8-membered ring, said ring comprising 0-3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl.

17. The compound of any one of the preceding claims, wherein the 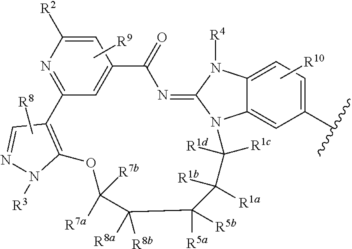
moiety is selected from

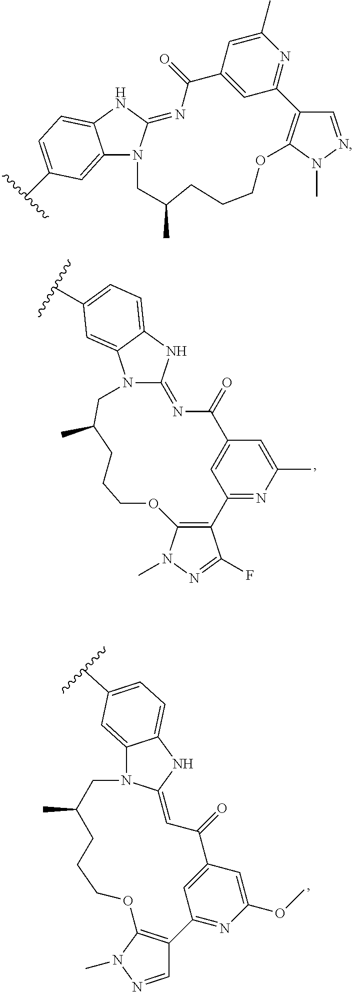

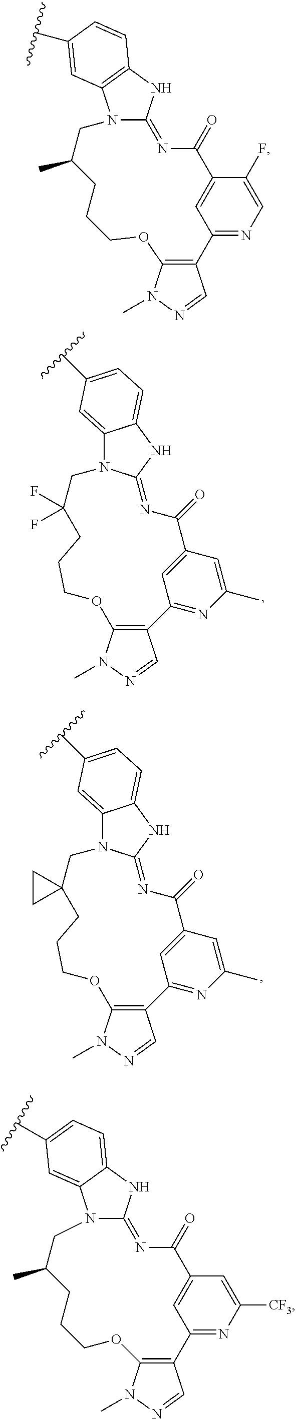

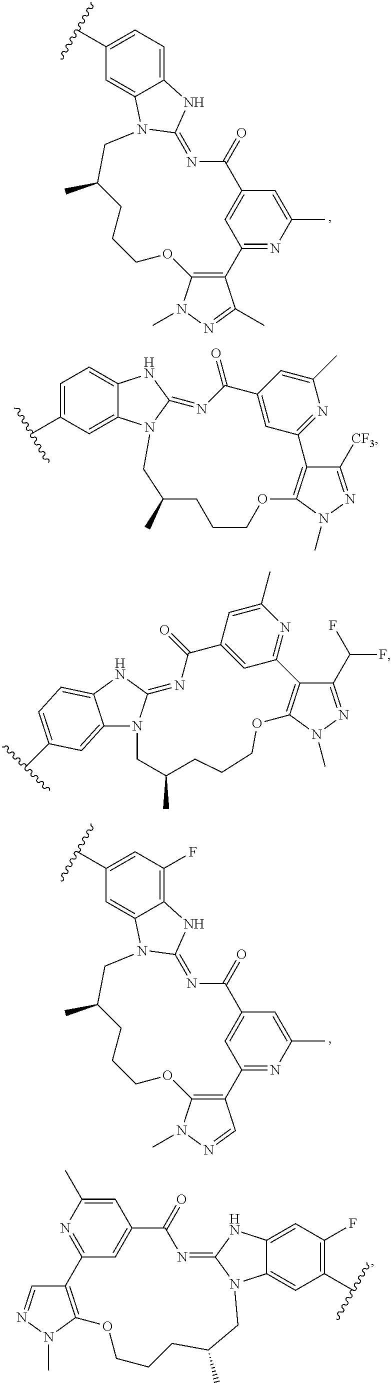

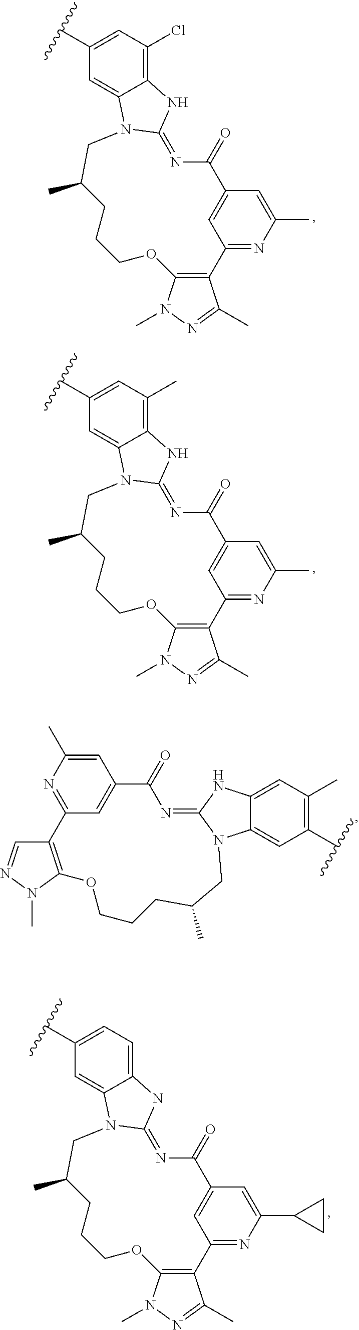

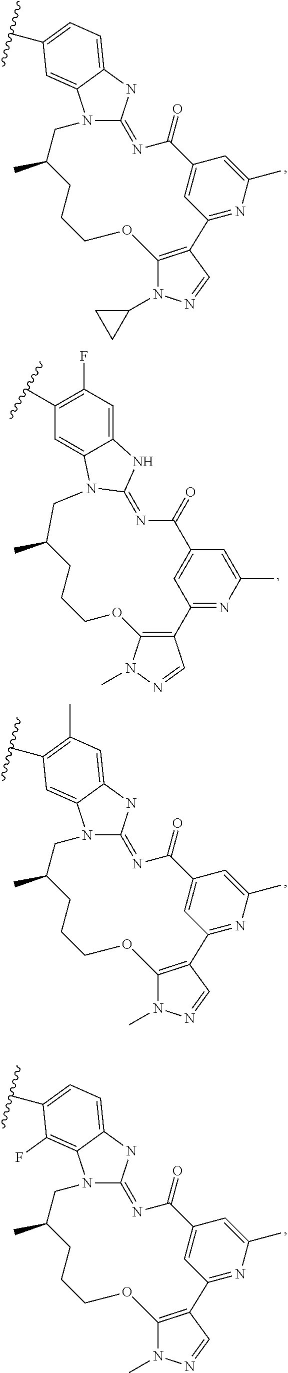

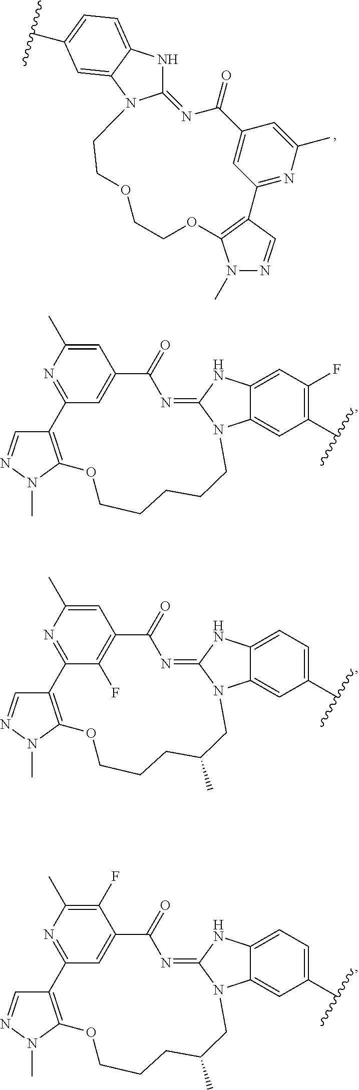 [SMILES: *c1ccc2c(c1)N1CCOCCOc3c(cnn3C)-c3cc(cc(C)n3)C(=O)/N=C/1N2 ; *c1cc2c(cc1F)N/C1=N\C(=O)c3cc(C)nc(c3)-c3cnn(C)c3OCCCCCN12 ; *c1ccc2c(c1)N1C[C@H](C)CCCOc3c(cnn3C)-c3nc(C)cc(c3F)C(=O)/N=C/1N2 ; *c1ccc2c(c1)N1C[C@H](C)CCCOc3c(cnn3C)-c3cc(c(F)c(C)n3)C(=O)/N=C/1N2]

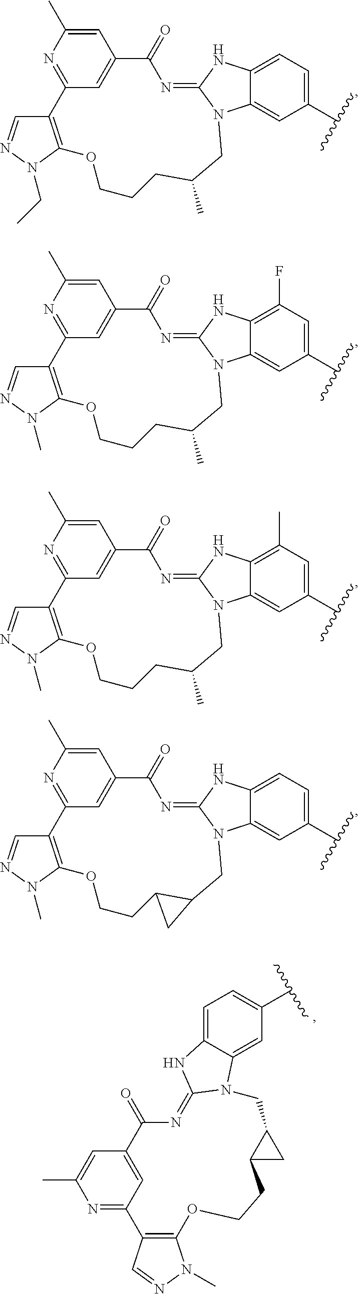

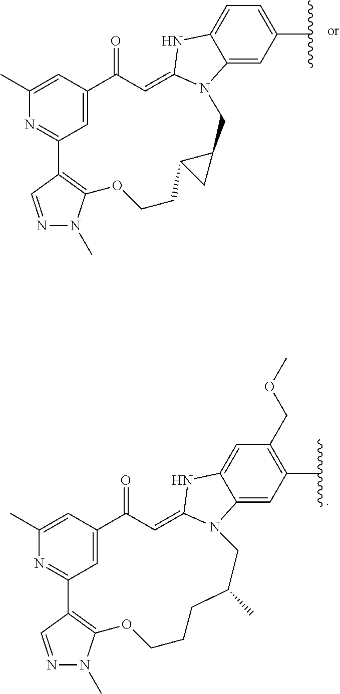 [SMILES: *c1ccc2c(c1)N1C[C@H]3C[C@@H]3CCOc3c(cnn3C)-c3cc(cc(C)n3)C(=O)/C=C/1N2 ; *c1cc2c(cc1COC)N/C1=C\C(=O)c3cc(C)nc(c3)-c3cnn(C)c3OCCC[C@@H](C)CN12]

18. The compound of any one of the preceding claims, wherein R^{11a}, R^{11b}, R^{11c}, R^{11d}, R^{12a}, R^{12b}, R^{12c}, and R^{12d} are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl, each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl is optionally substituted with at least one substituent selected from hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN: or
(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur; said ring is optionally substituted with at least one substituent selected from hydrogen, halogen, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN.

19. The compound of any one of the preceding claims, wherein R^{11a}, R^{11b}, R^{11c}, R^{11d}, R^{12a}, R^{12b}, R^{12c} and R^{12d} are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl: preferably, R^{11a}, R^{11b}, R^{11c}, R^{11d}, R^{12a}, R^{12b}, R^{12c} and R^{12d} are each independently hydrogen, F, Cl, Br, I, methyl, ethyl or propyl; more preferably, R^{11a}, R^{11b}, R^{11c}, R^{11d}, R^{12a}, R^{12b}, R^{12c} and R^{12d} are each independently hydrogen or methyl: or
(R^{11a} and R^{12a}), (R^{11b} and R^{12b}), (R^{11c} and R^{12c}) or (R^{11d} and R^{12d}) together with the carbon atoms to which they are attached, form a 3-, 4-, 5-, 6-, 7- or 8-membered ring, said ring comprising 0, 1, 2, or 3 heteroatoms independently selected from nitrogen, oxygen or sulfur; preferably (R^{11a} and R^{12a}), (R^{11b} and R^{12b}), (R^{11c} and R^{12c}) or (R^{11d} and R^{12d}) together with the carbon atoms to which they are attached, form a 3-, 4- or 5-membered cycloalkyl ring.

20. The compound of any one of the preceding claims, wherein L¹ is selected from —O—, —C(O)—, —N(R^{a})—, *^{L1}—C(O)N(R^{a})—**^{L1}, *^{L1}C(O)O—**^{L1}, *^{L1}—N(R^{a})C(O)—**^{L1}, *^{L1}-O(O)-**^{L1}, 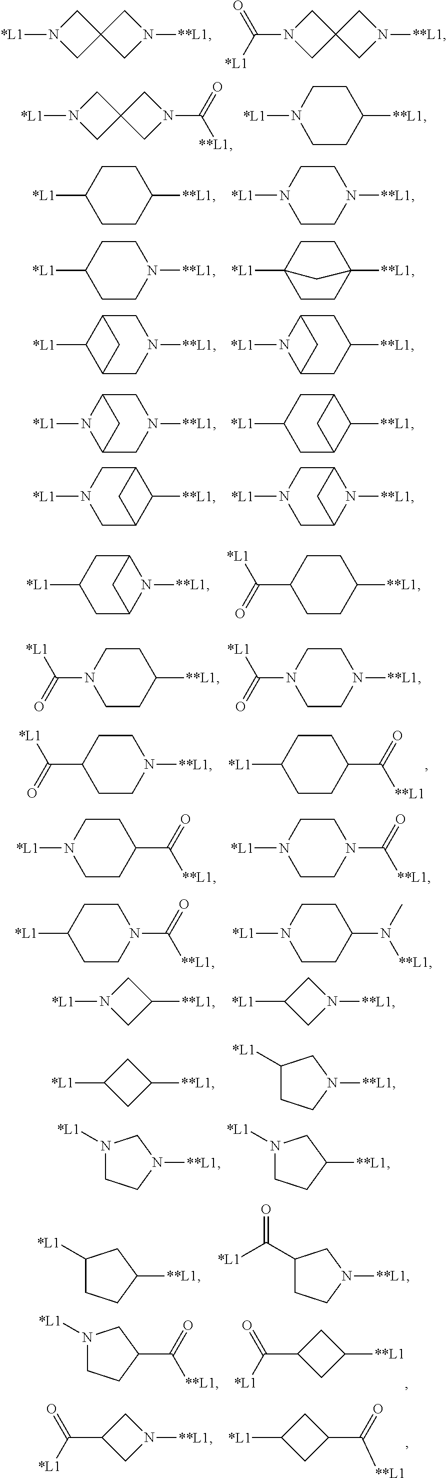 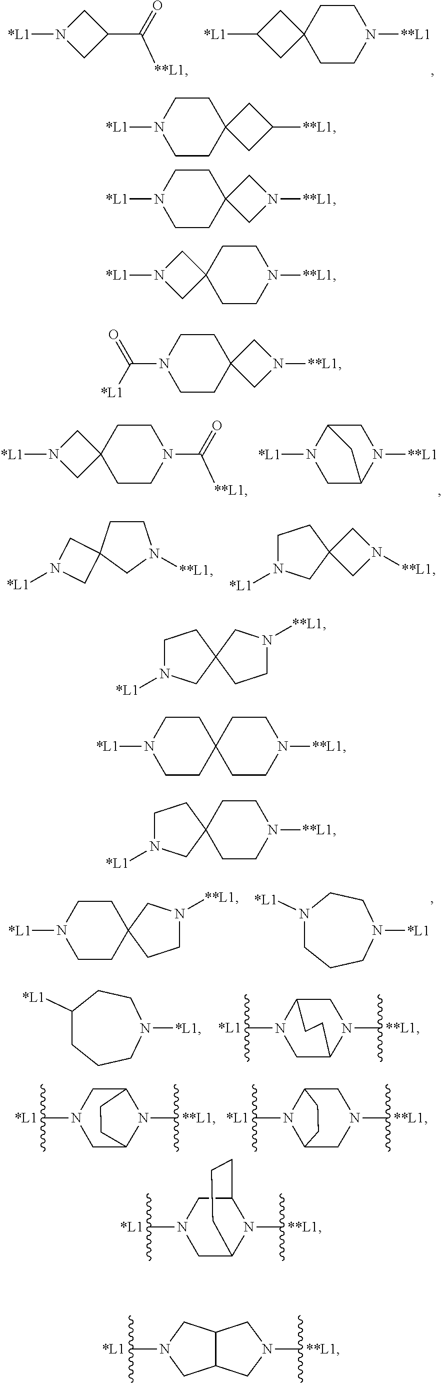 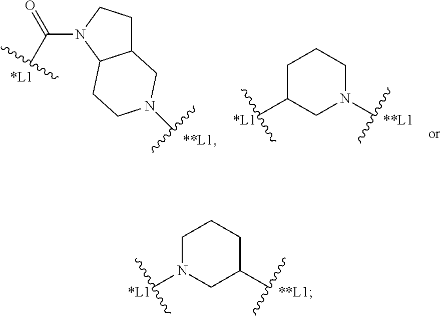 [SMILES: *C(=O)N1CCC2CN(*)CCC21 ; *C1CCCN(*)C1 ; *C1CCCN(*)C1]
wherein each of said 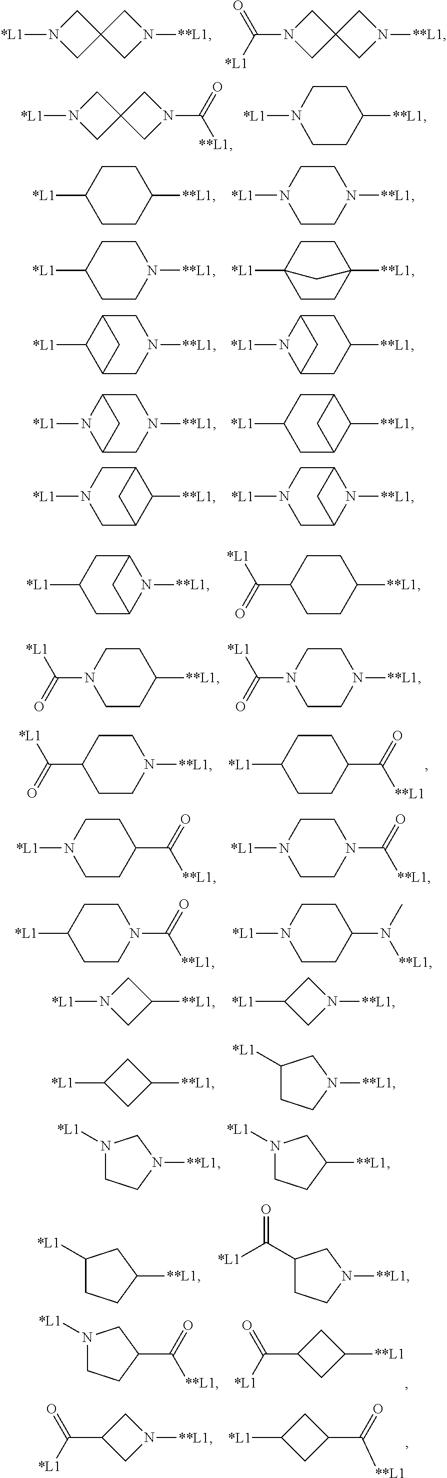 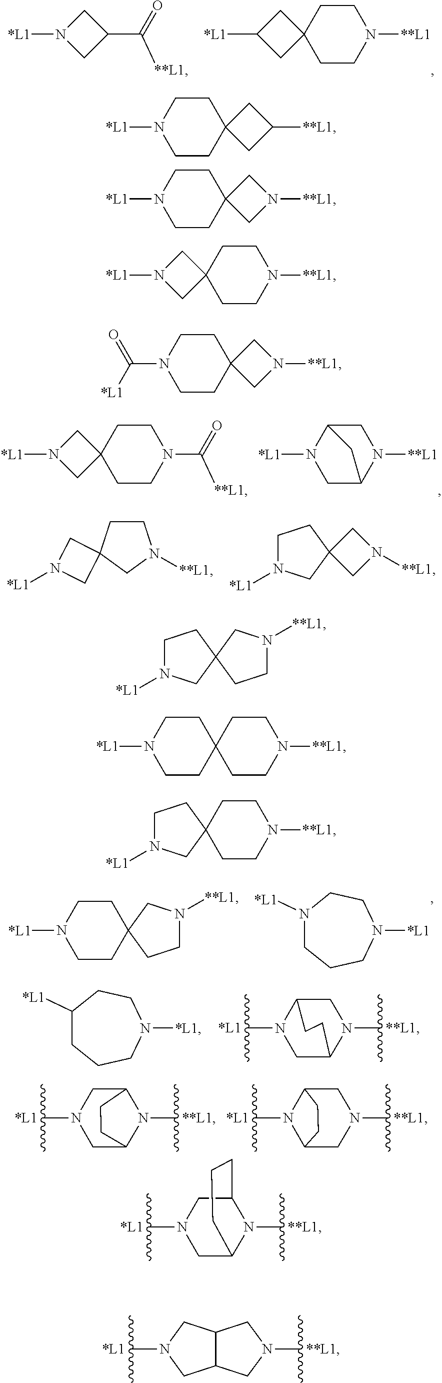 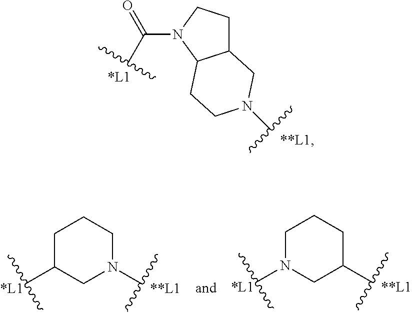 [SMILES: *C(=O)N1CCC2CN(*)CCC21 ; *C1CCCN(*)C1 ; *C1CCCN(*)C1]
is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

21. The compound of any one of the preceding claims, wherein L¹ is selected from —O—, —N(CH₃)—, —C(O)—, —NH—, *^{L1}—C(O)N(CH₃)—**^{L1}, *^{L1}—C(O)NH—**^{L1}, *^{L1}—C(O)—**^{L1}, *^{L1}—C(O)N(C₂H₈)—**^{L1}, *^{L1}—C(O)N(C₃H₇)—**^{L1}, *^{L1}—N(CH₃)C(O)—**^{L1}, *^{L1}—NHC(O)—**^{L1}, *^{L1}-(C(O)—**^{L1}, *^{L1}—N(C₂H₈)C(O)—**^{L1}, *^{L1}—N(C₃H₇)C(O)—**^{L1},

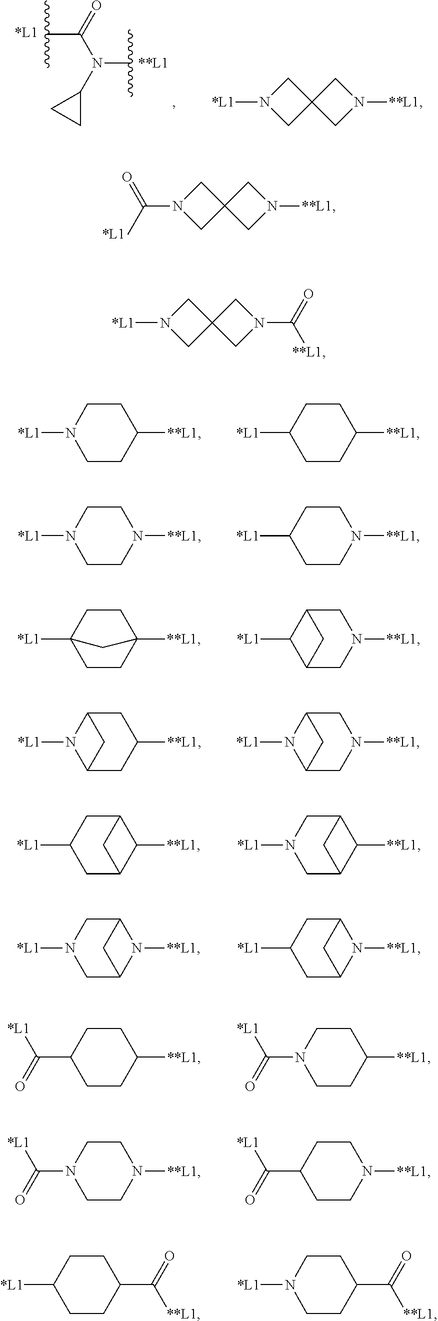

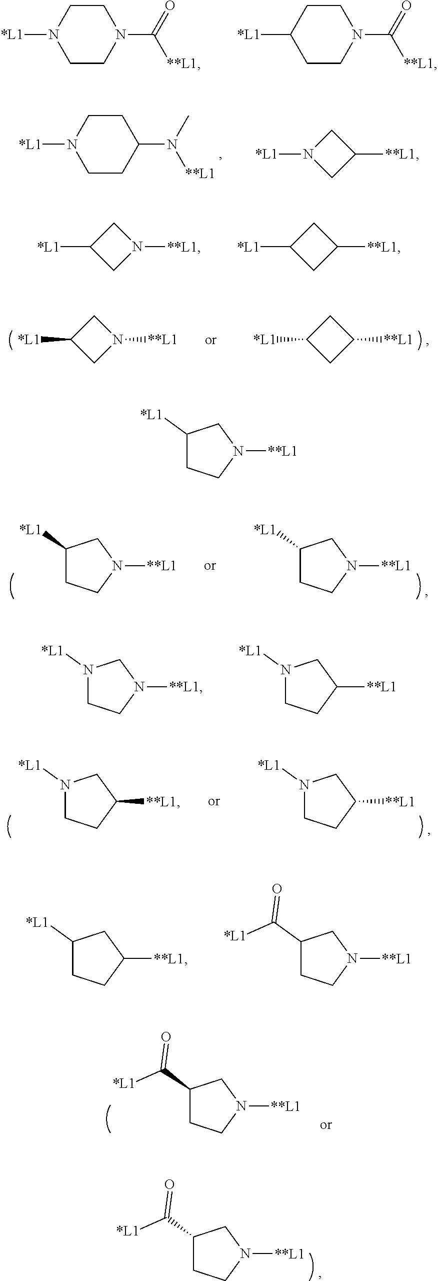

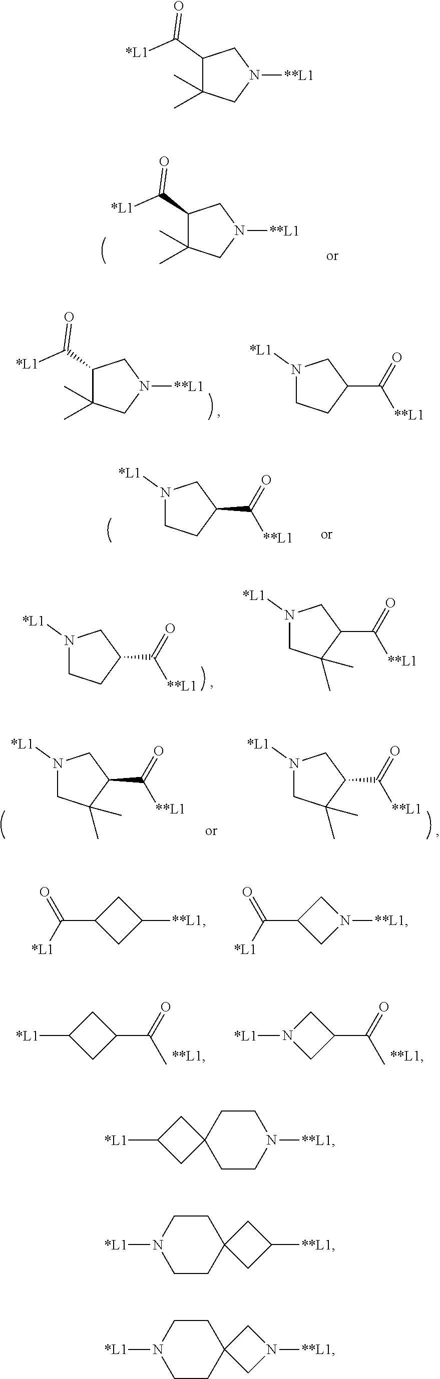

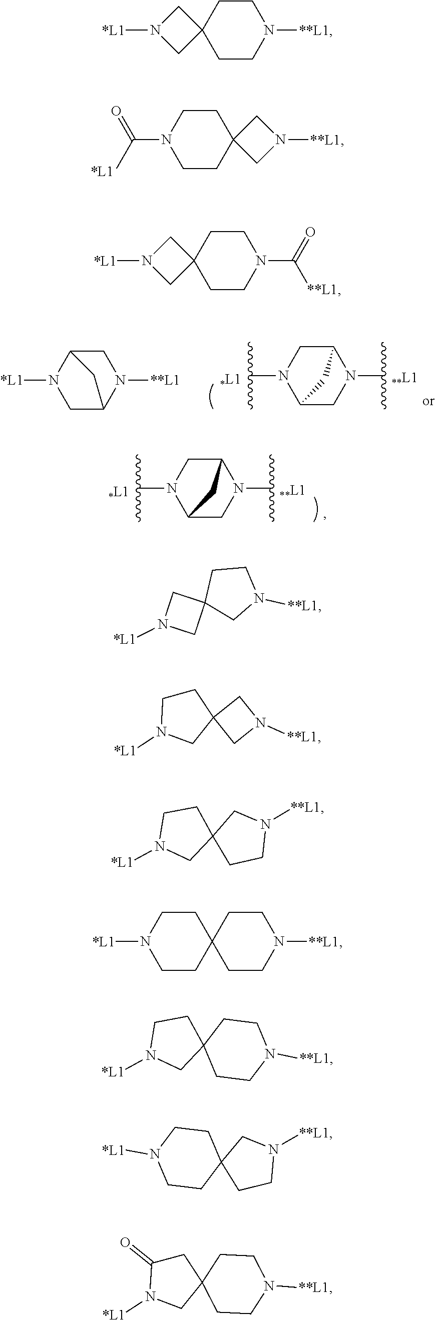

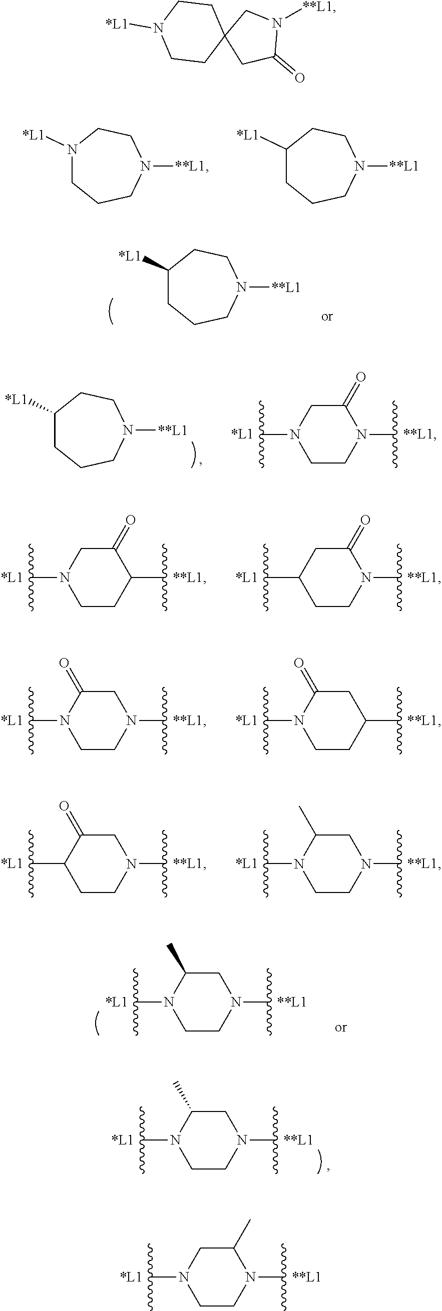

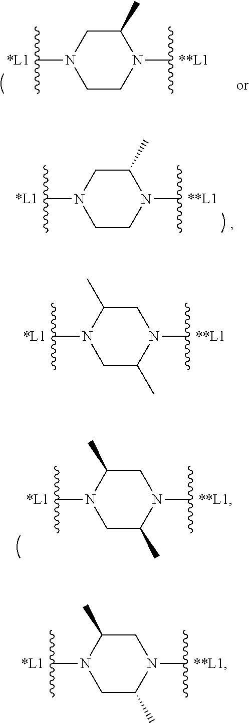

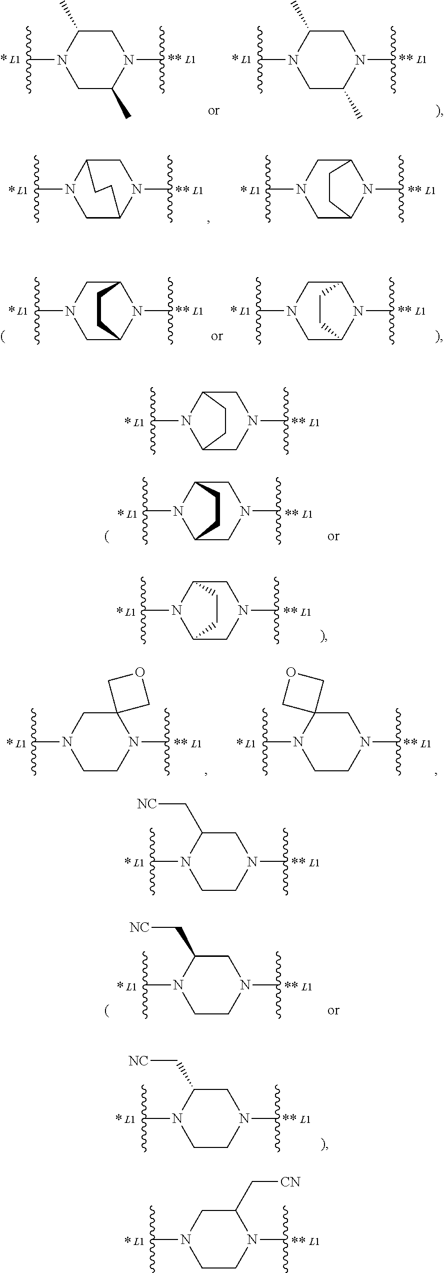

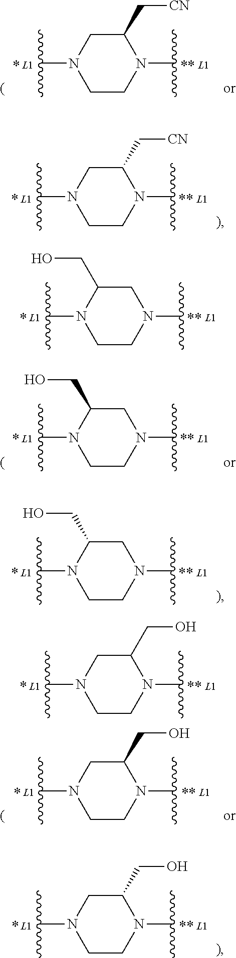

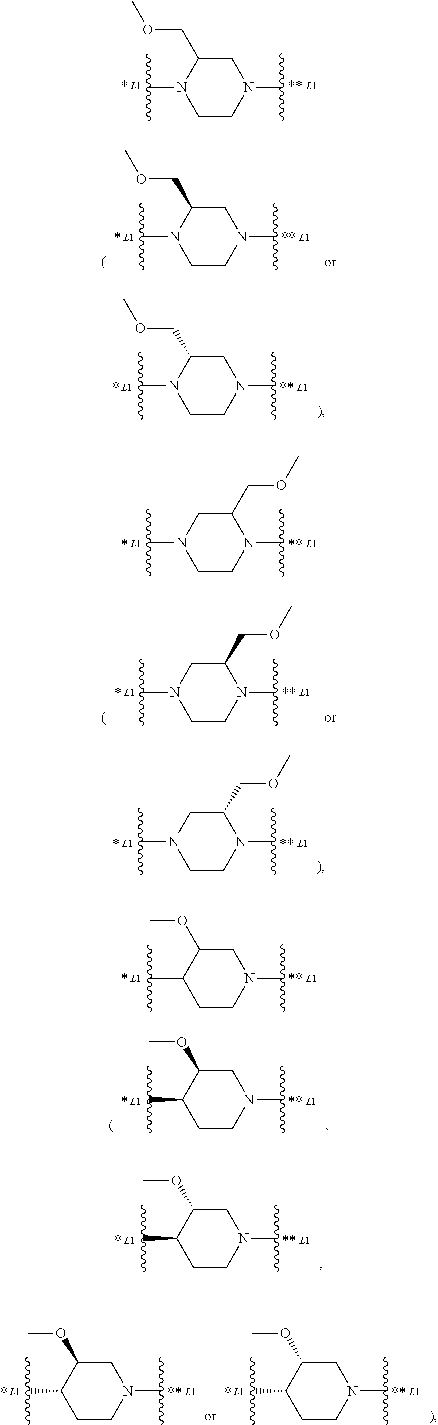

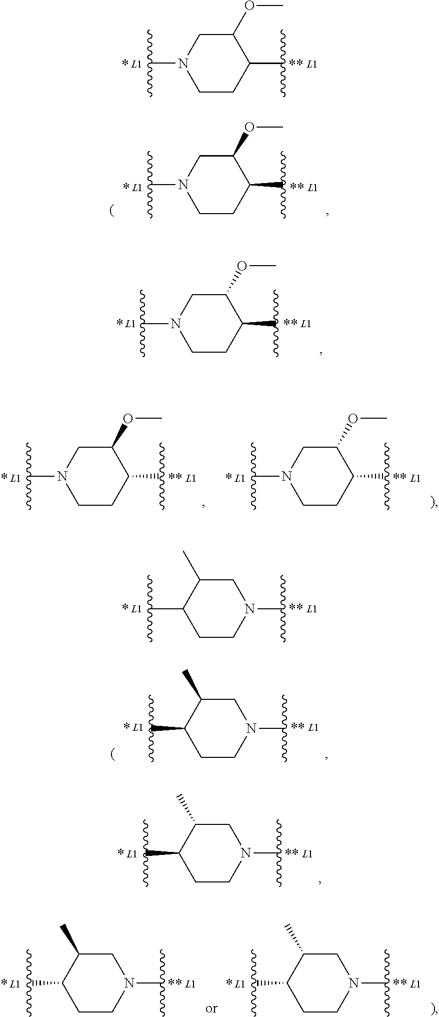

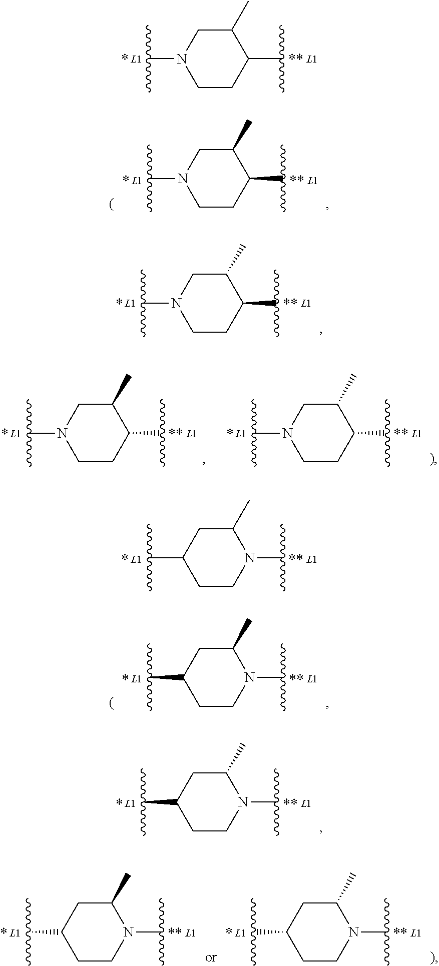

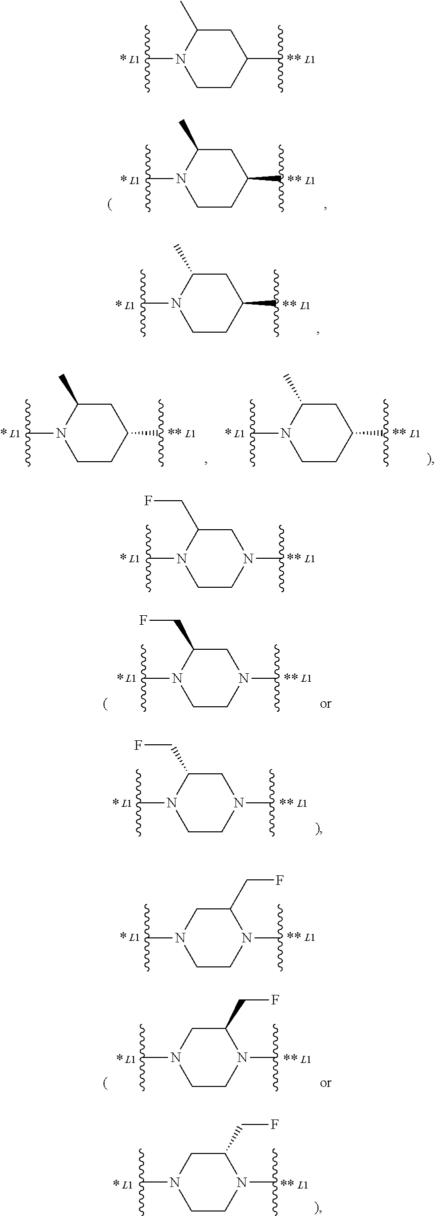

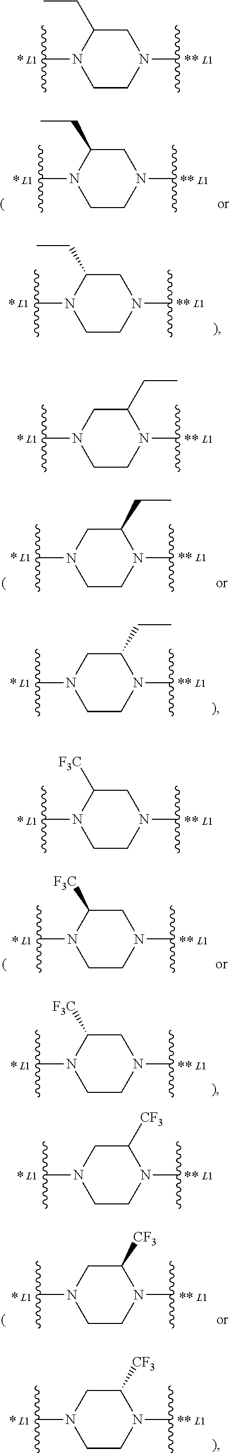

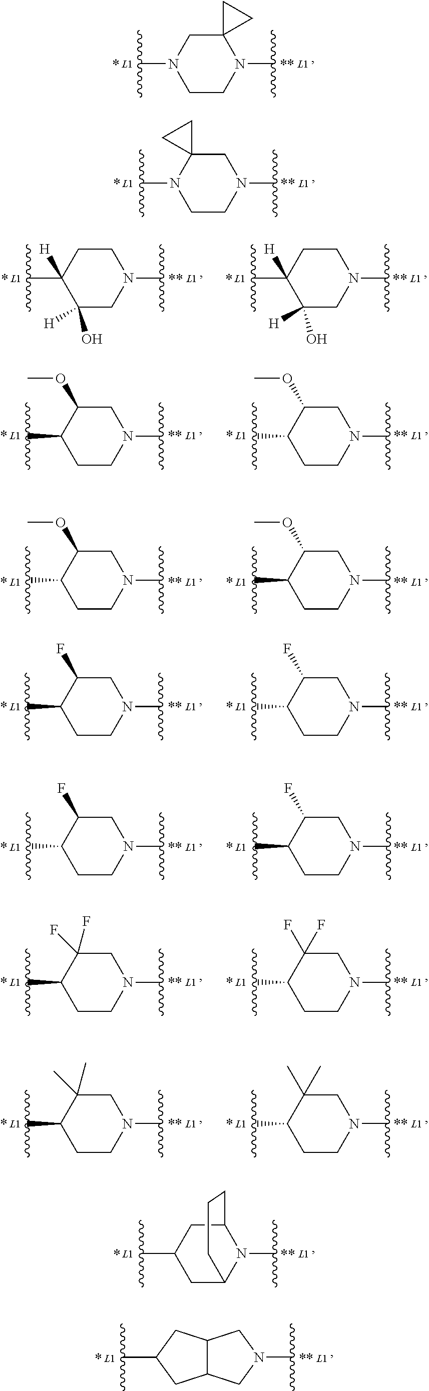

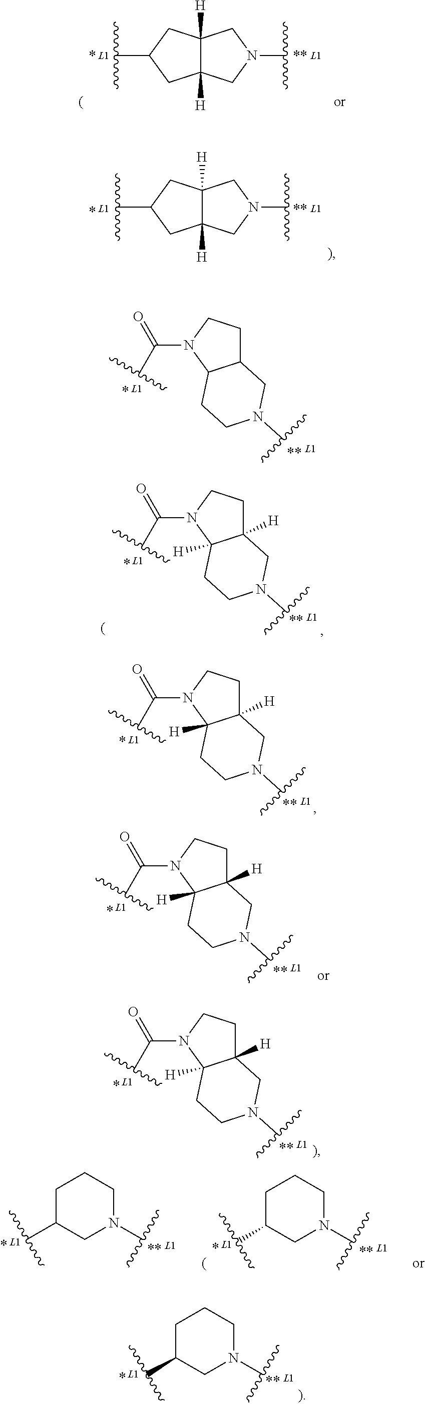

22. The compound of any one of the preceding claims, wherein L² is selected from —O—, —C(O)—, —N(R^{a})—, *^{L2}—C(O)N(R^{a})—**^{L2}, *^{L2}—C(O)O—**^{L2}, *^{L2}—N(R^{a})C(O)—**^{L2}, *^{L2}—OC(O)—**^{L2}, 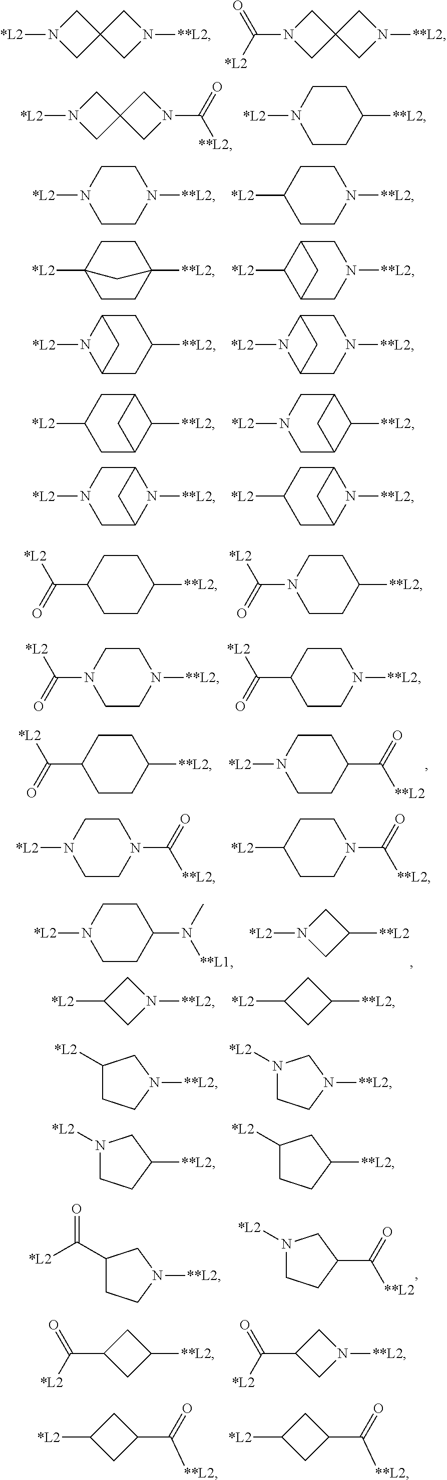 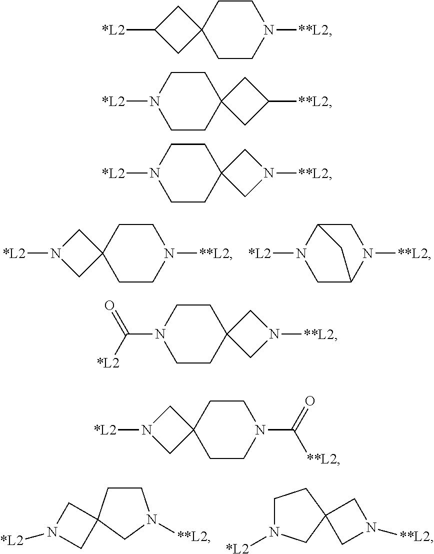 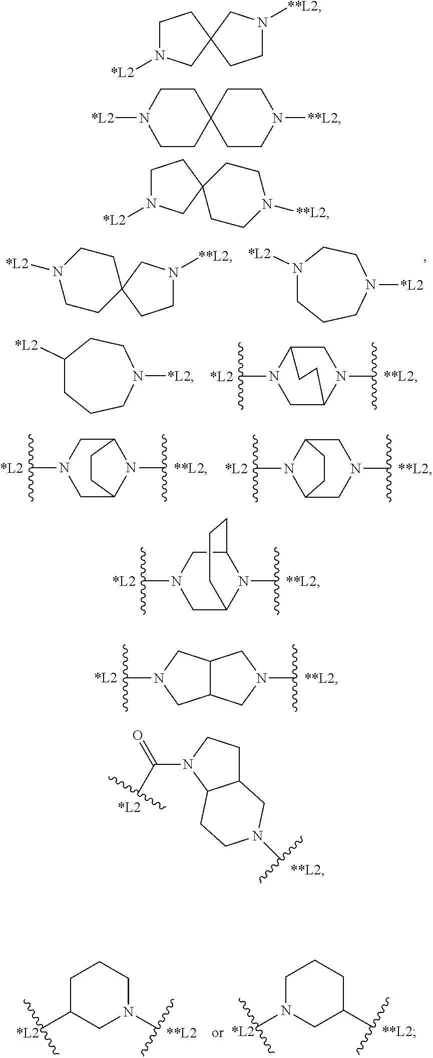
wherein each of said 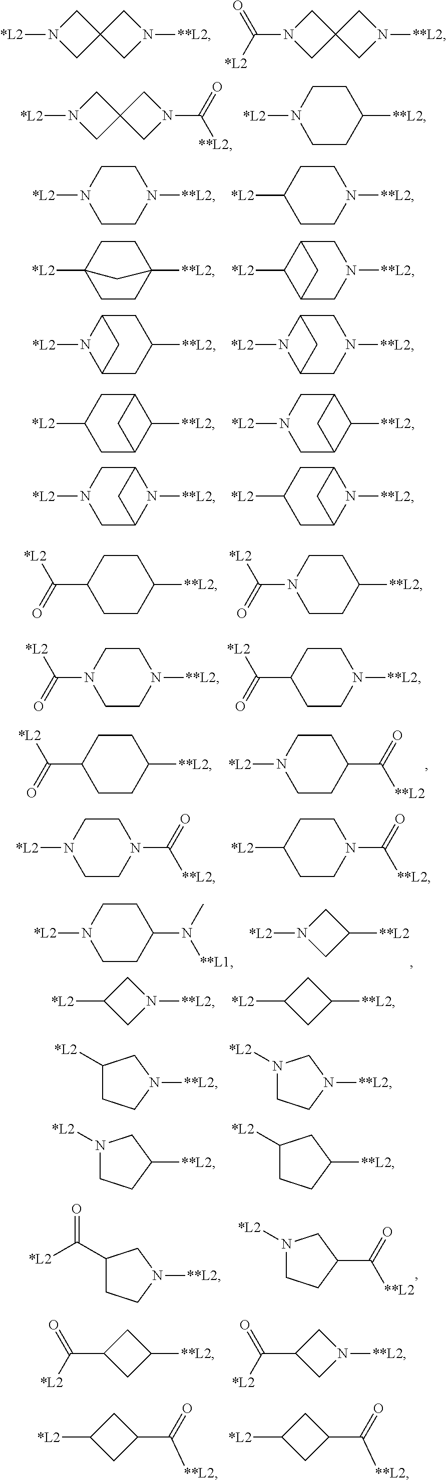 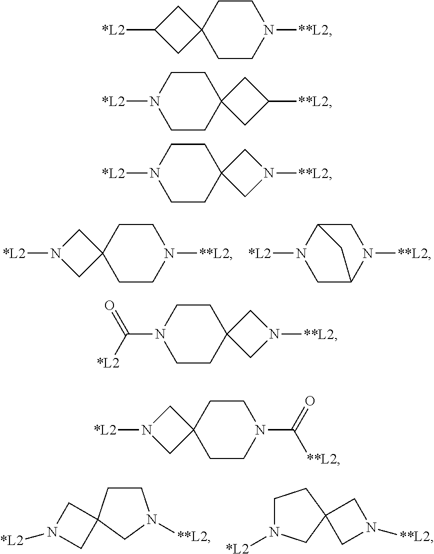 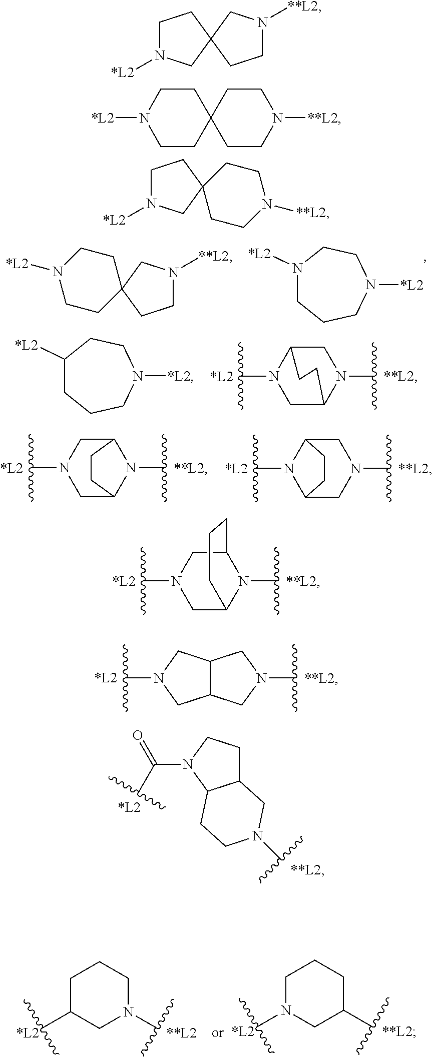
is optionally substituted with at least one R^{L2c},
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur, said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

23. The compound of any one of the preceding claims, wherein L² is selected from —O—, —N(CH₃)—, —C(O)—, —NH—, *^{L2}—C(O)N(CH₃)—**^{L2}, *^{L2}—C(O)NH—**^{L2}, *^{L2}—C(O)O—**^{L2}, *^{L2}—C(O)N(C₂H₈)—**^{L2},*^{L2}—C(O)N(C₃H₇)—**^{L2}, *^{L2}—N(CH₃)C(O)—**^{L2}, *^{L2}—NHC(O)—**^{L2}, *^{L2}—OC(O)—**^{L2}, *^{L2}—N(C₂H₅)C(O)—**^{L2}, *^{L2}N(C₃H⁷)C(O)—**^{L2},

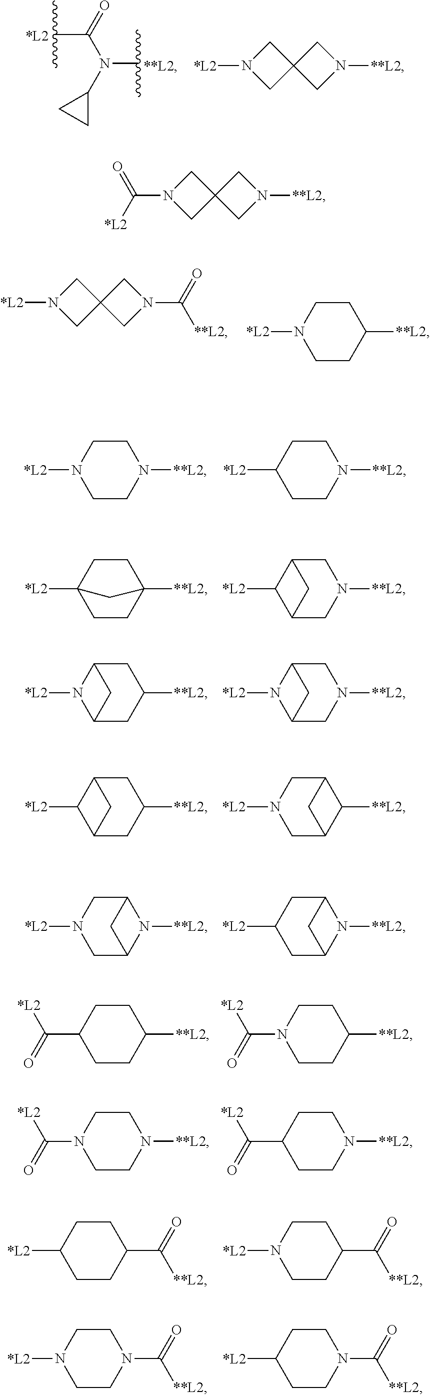

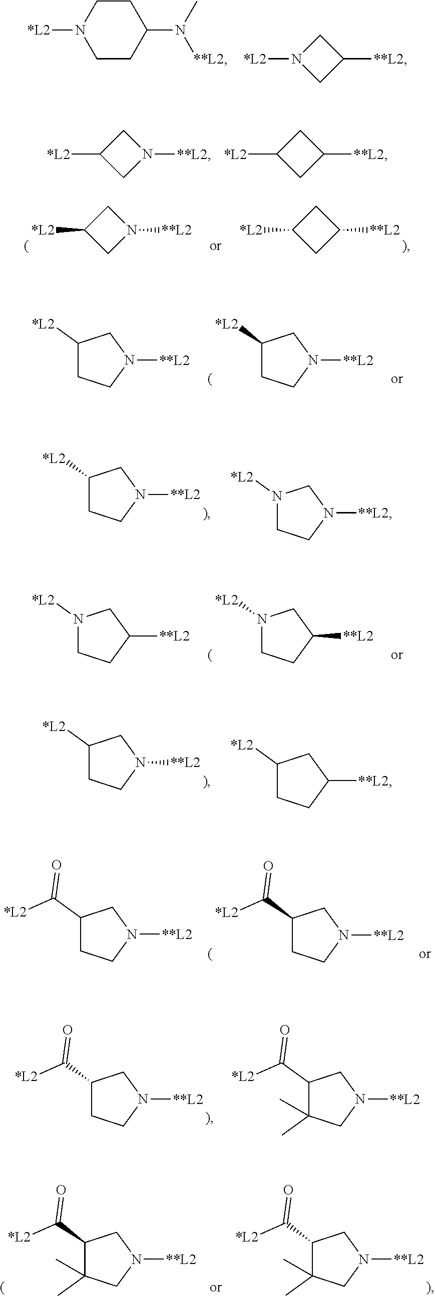

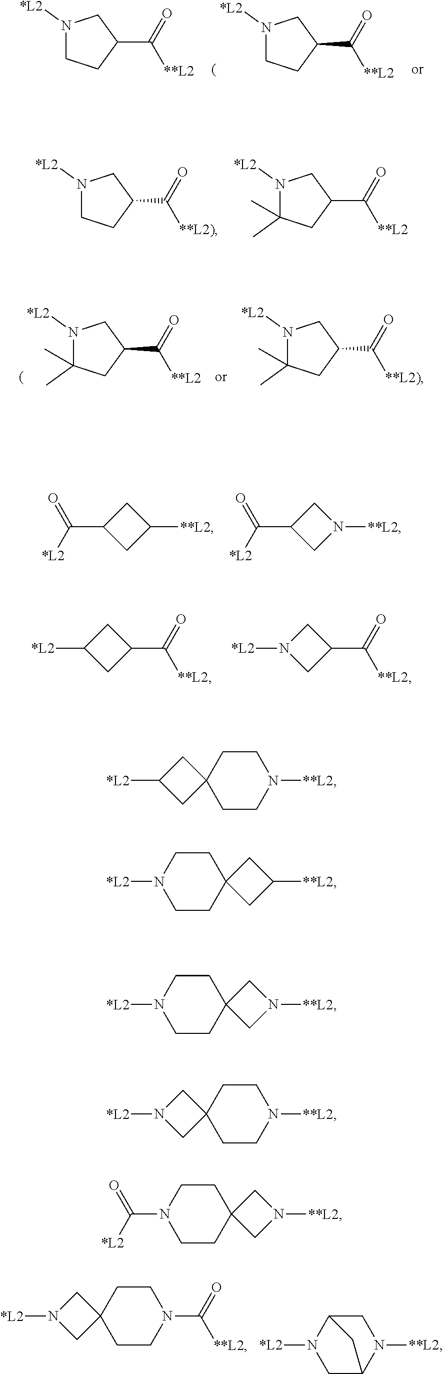

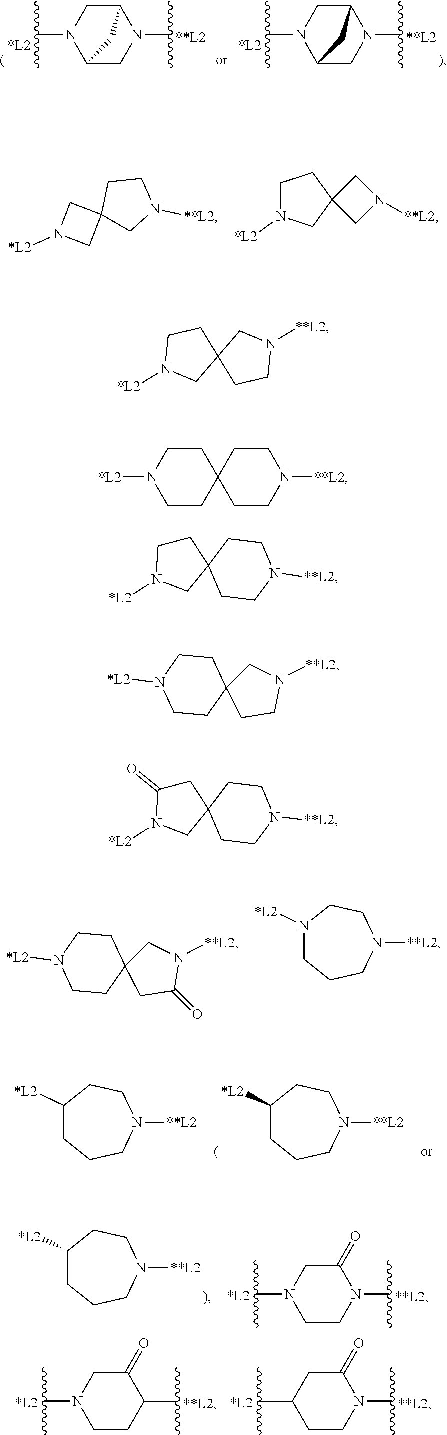

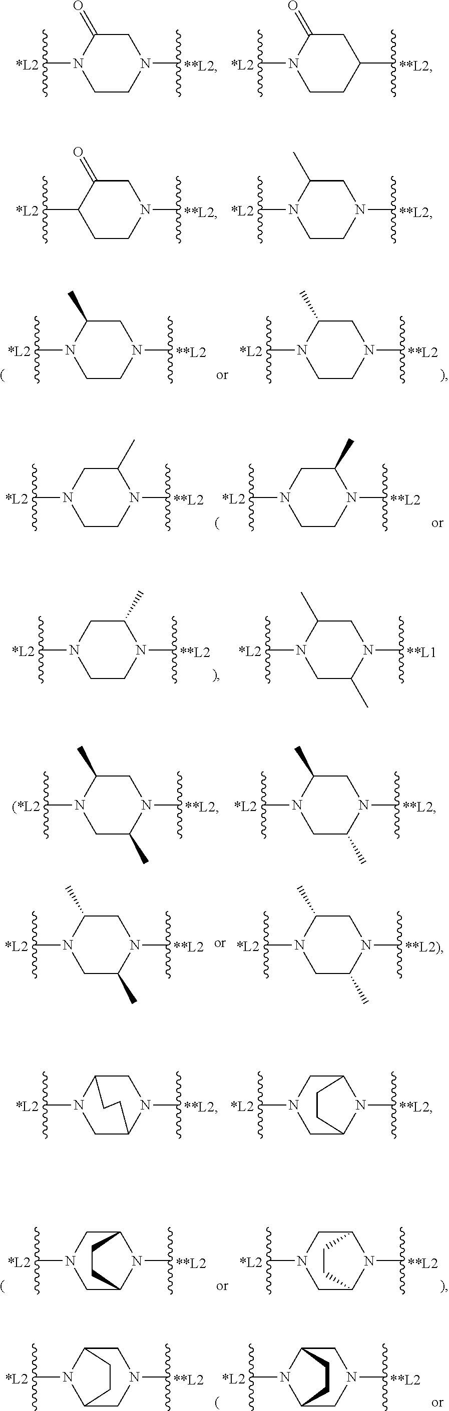

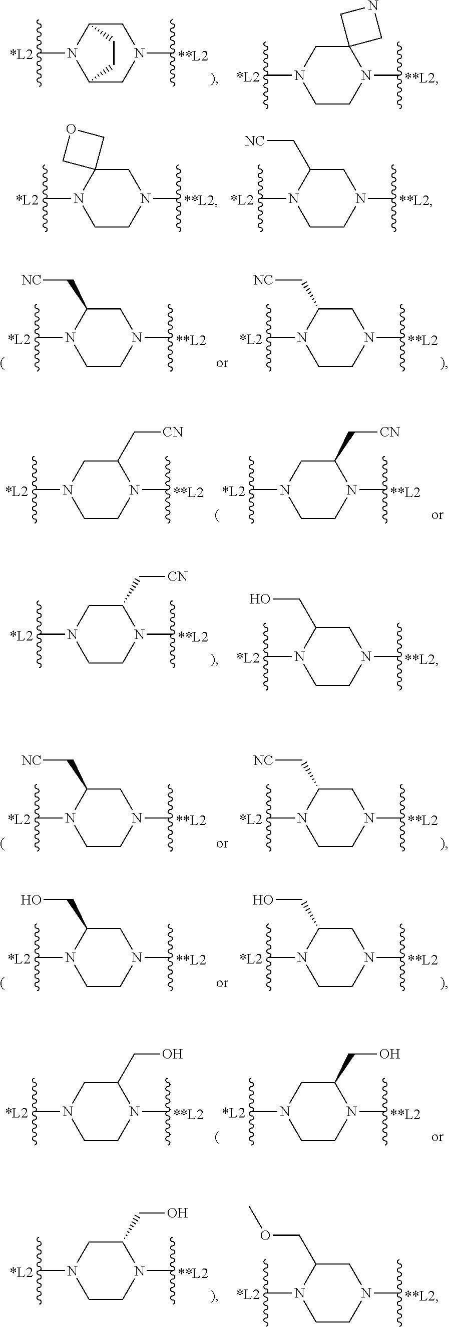

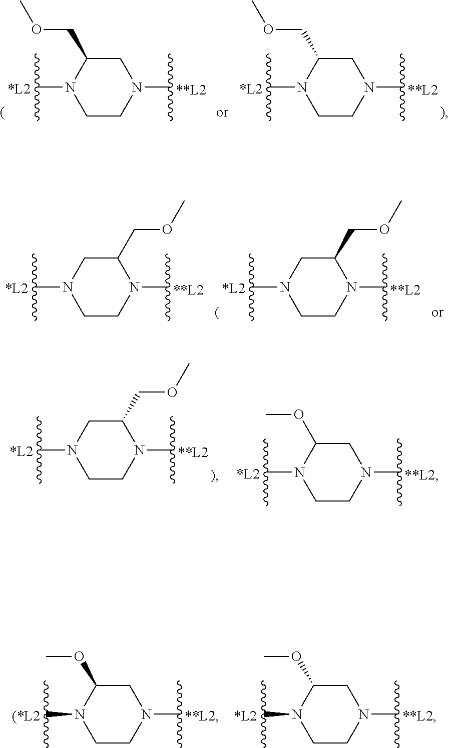

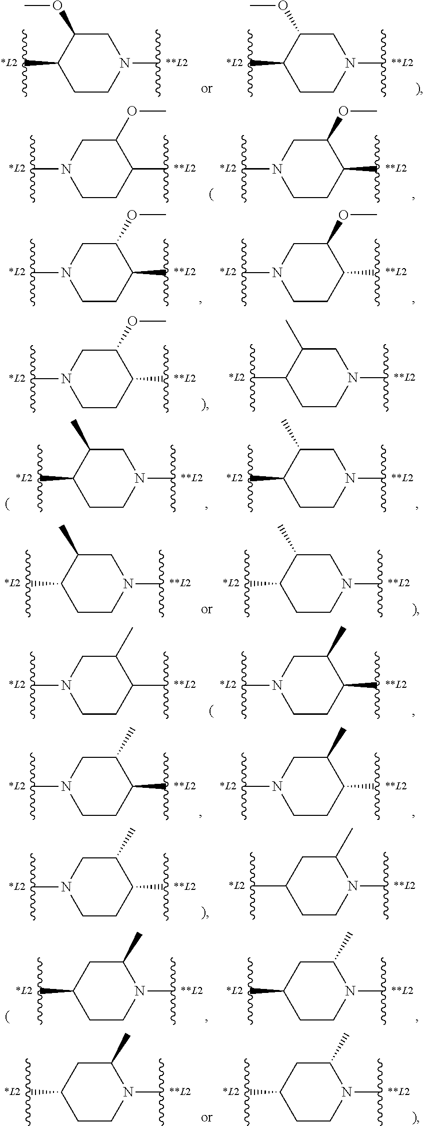

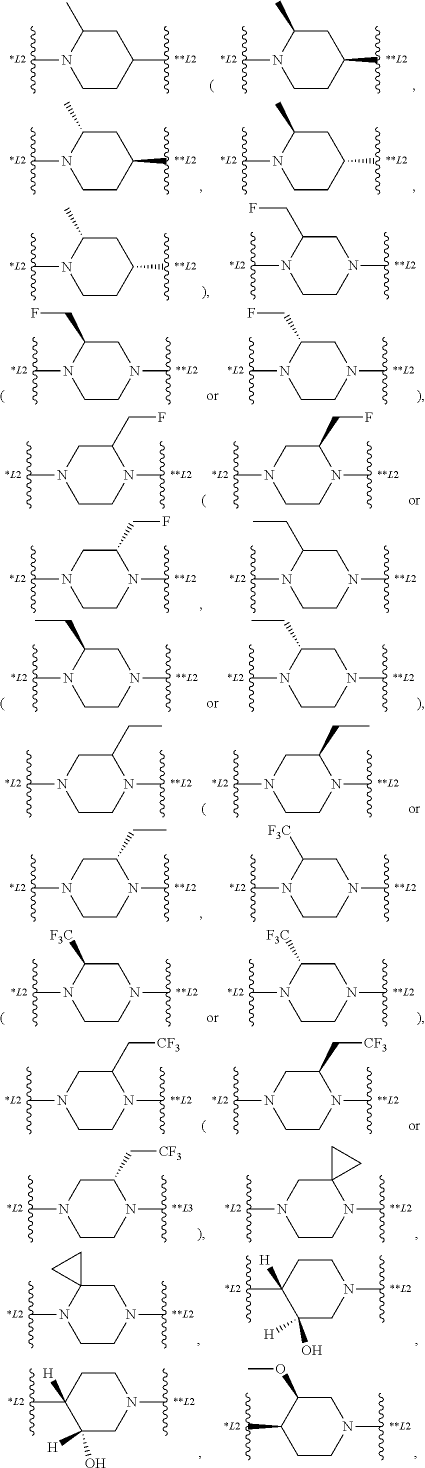

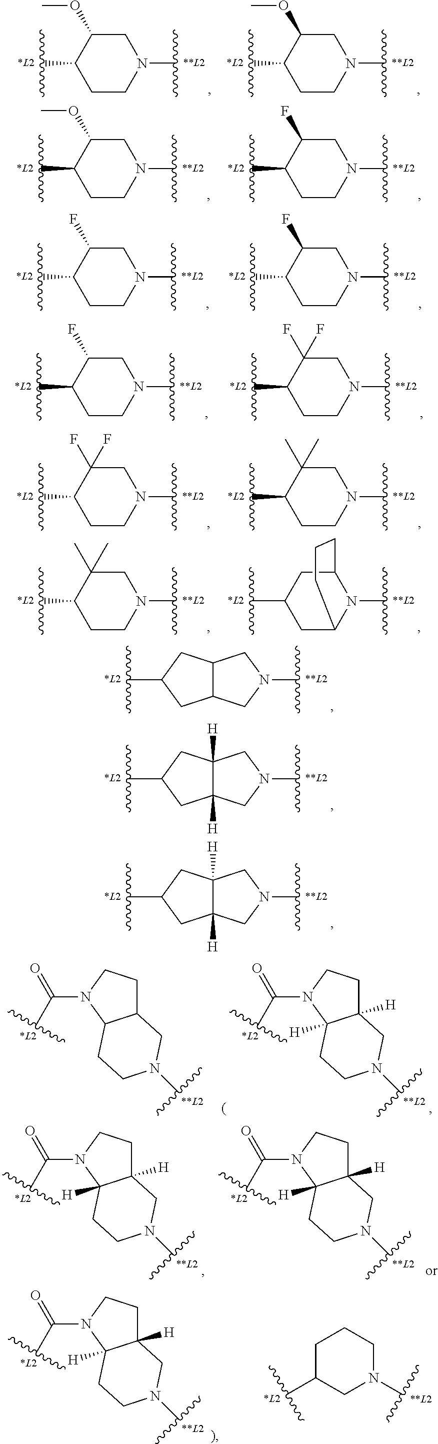

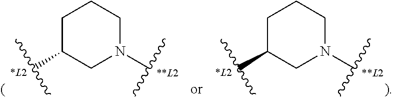 [SMILES: *[C@@H]1CCCN(*)C1 ; *[C@H]1CCCN(*)C1]

24. The compound of any one of the preceding claims, wherein L³ is selected from —O—, —N(R^{a})—, —C(O)—, *^{L3}—C(O)N(R^{a})—**^{L3}, *^{L3}—C(O)O—**^{L3}, *^{L3}—N(R^{a})C(O)—**^{L3}, *^{L3}—OC(O)—**^{L3}, 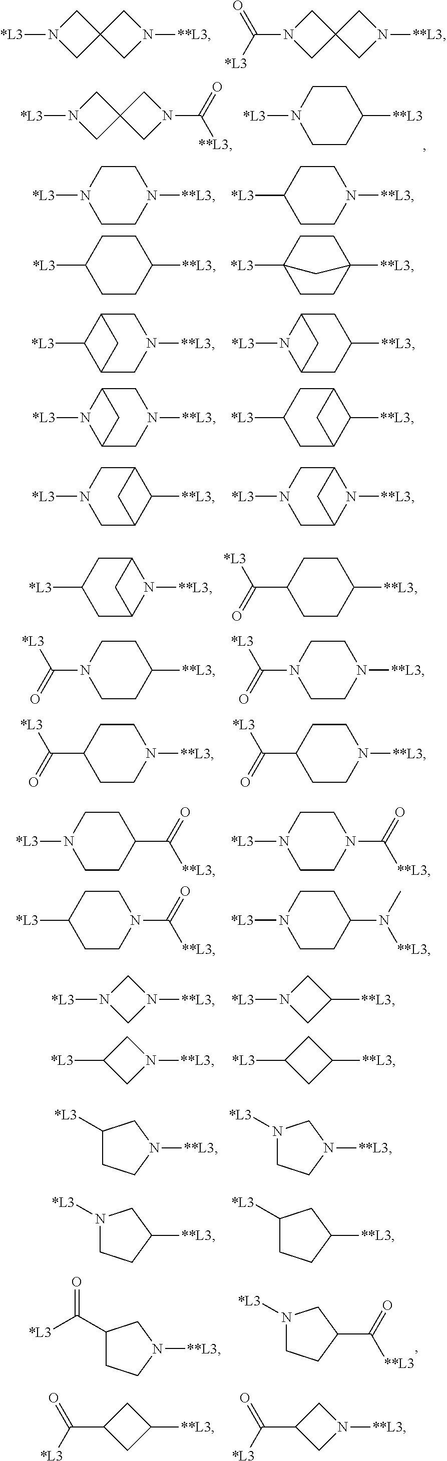 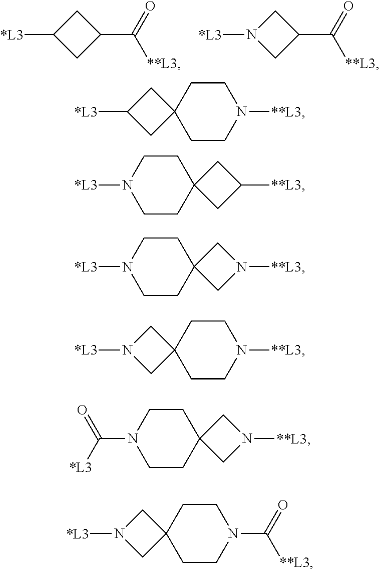 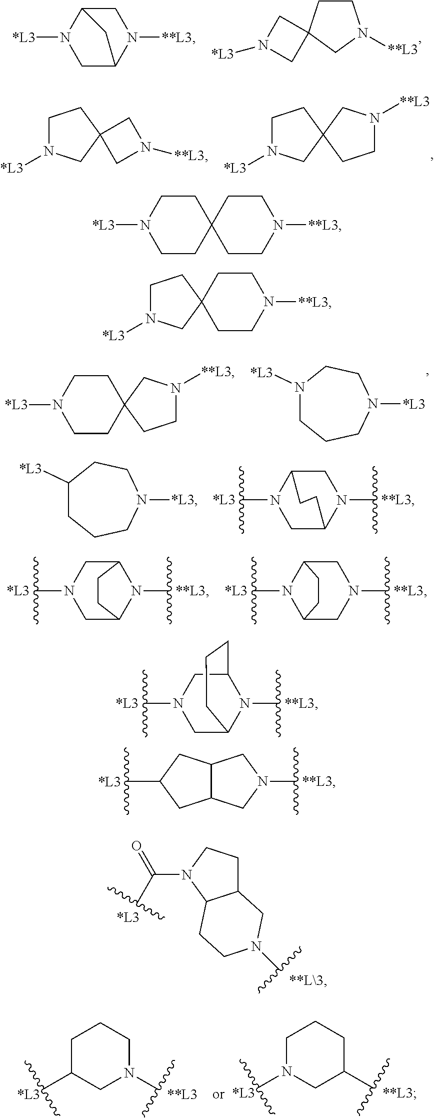
wherein each of said 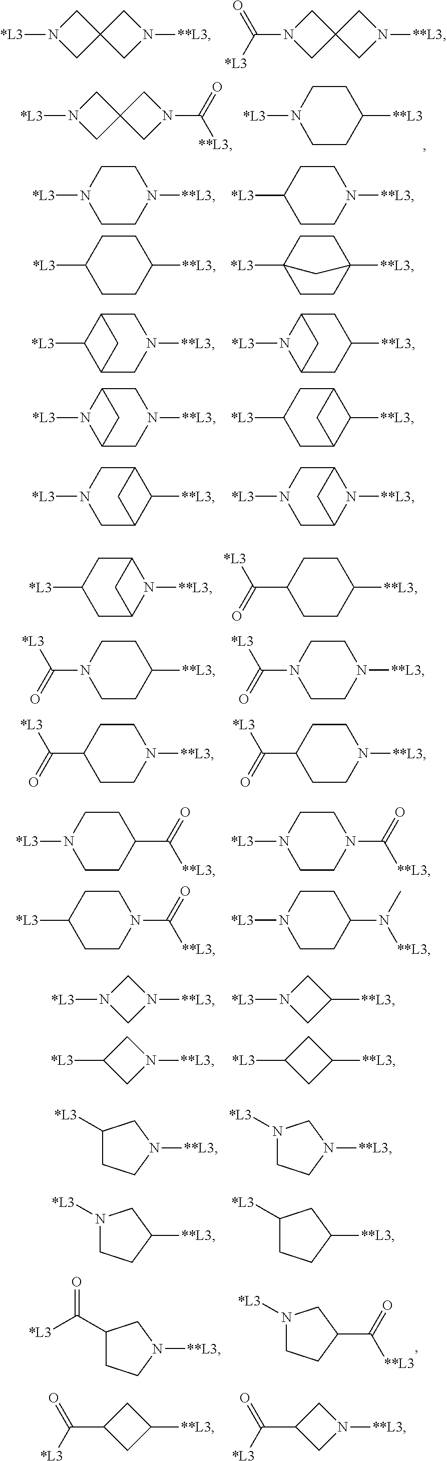 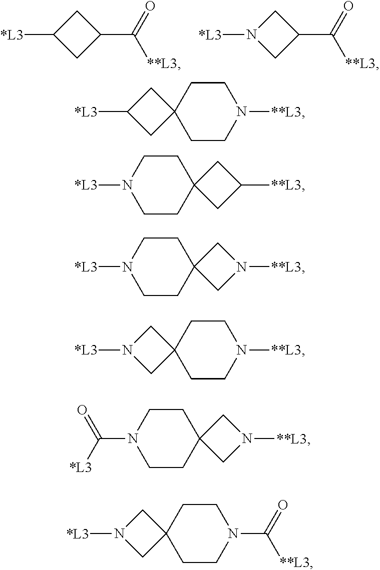 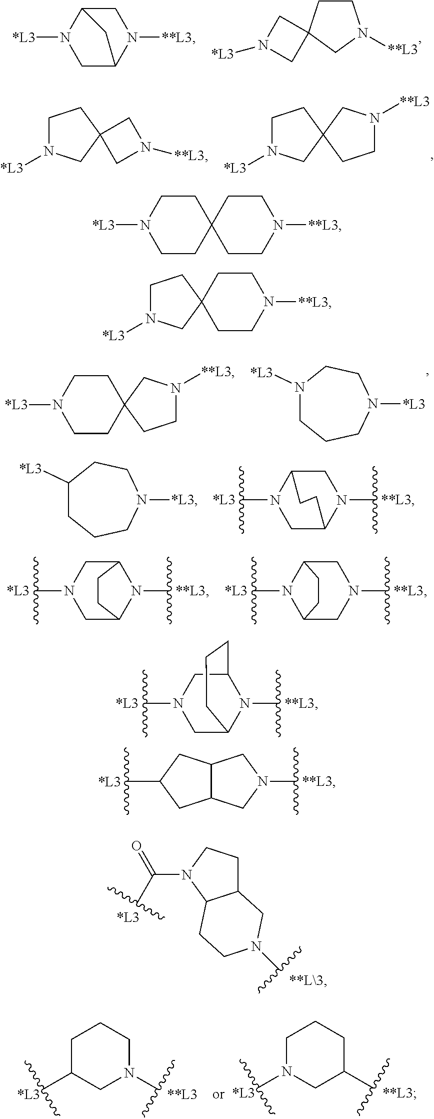
is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl:
each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}, R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

25. The compound of any one of the preceding claims, wherein L³ is selected from —O—, —N(CH₃)—, —C(O)—, —NH—, *^{L3}—C(O)N(CH₃)—**^{L3}, *^{L3}—C(O)NH—**^{L3}-; *^{L3}—C(O)O—**^{L3}, *^{L3}—C(O)N(C₂H₇)—**^{L3}, *^{L3}—C(O)N(C₃H₇)—**^{L3}, *^{L3}—N(CH₃)C(O)—**^{L3}, *^{L3}—NHC(O)—**^{L3}-; *^{L3}—OC(O)—**^{L3}, *^{L3}—N(C₂H₅)C(O)—**^{L3}, *^{L3}—N(C₃H₇)C(O)—**^{L3},

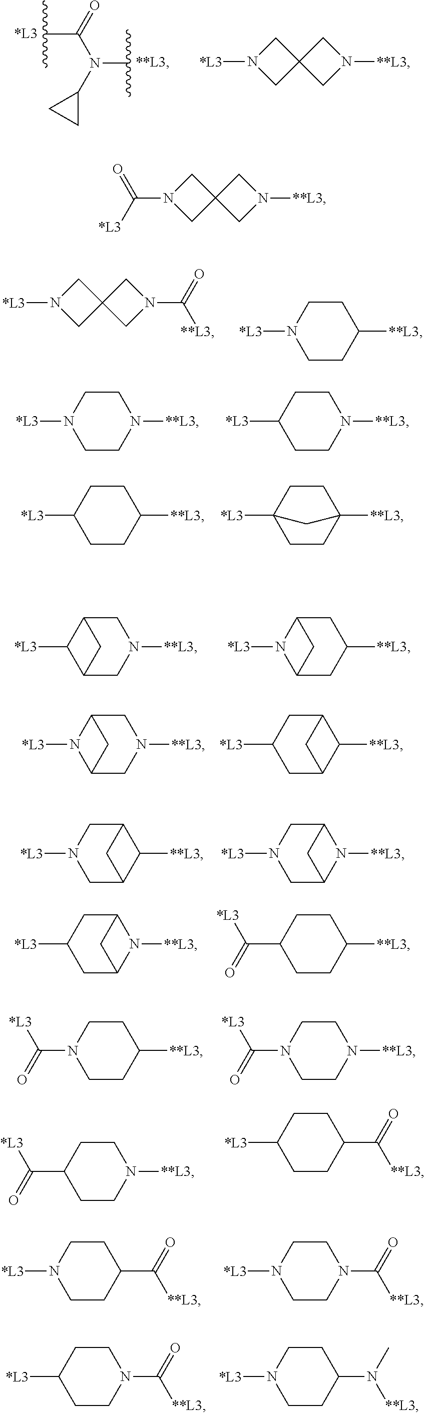

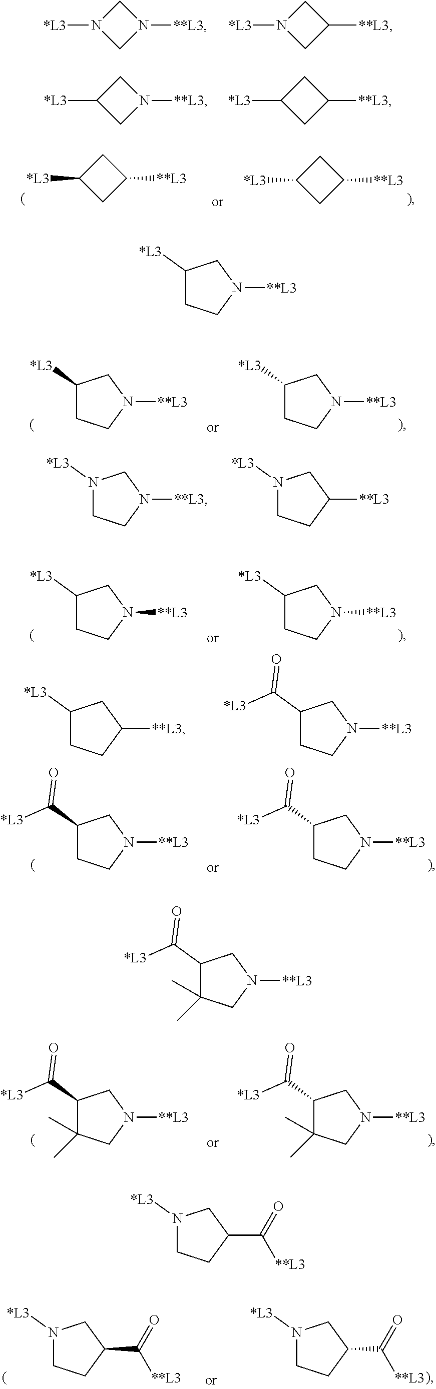

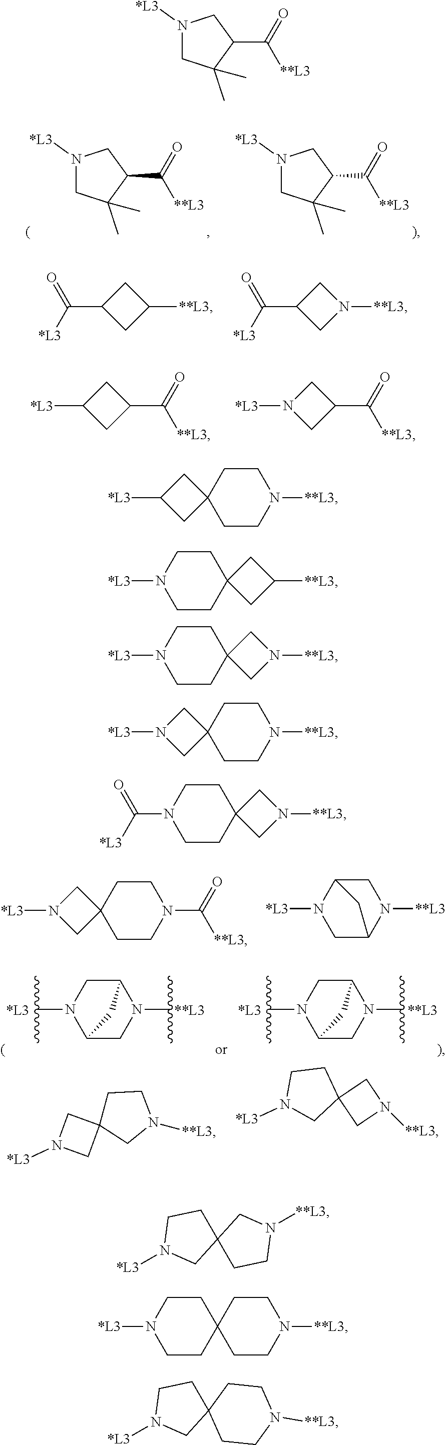

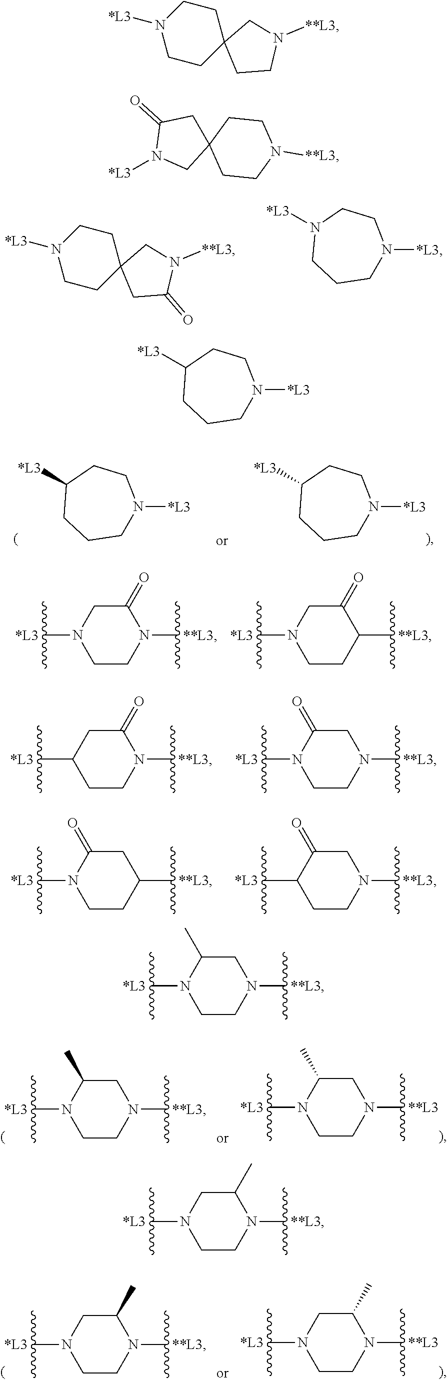

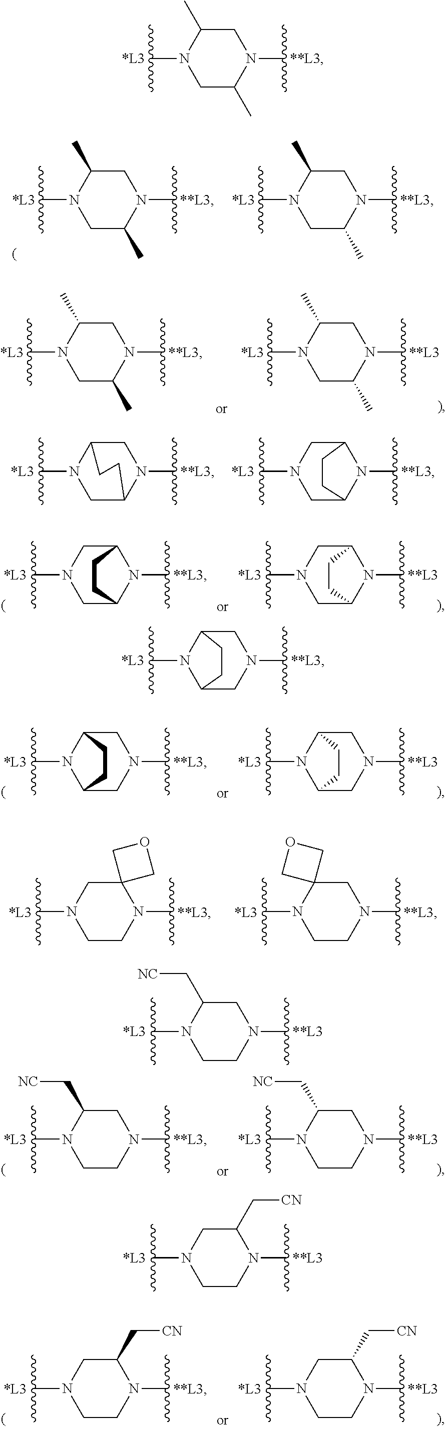

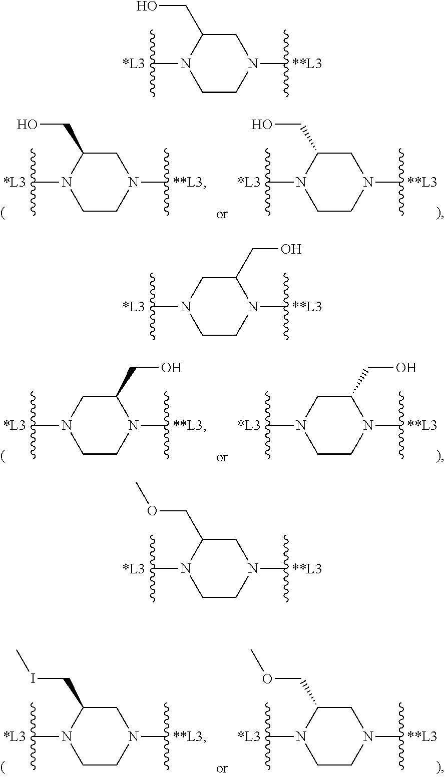

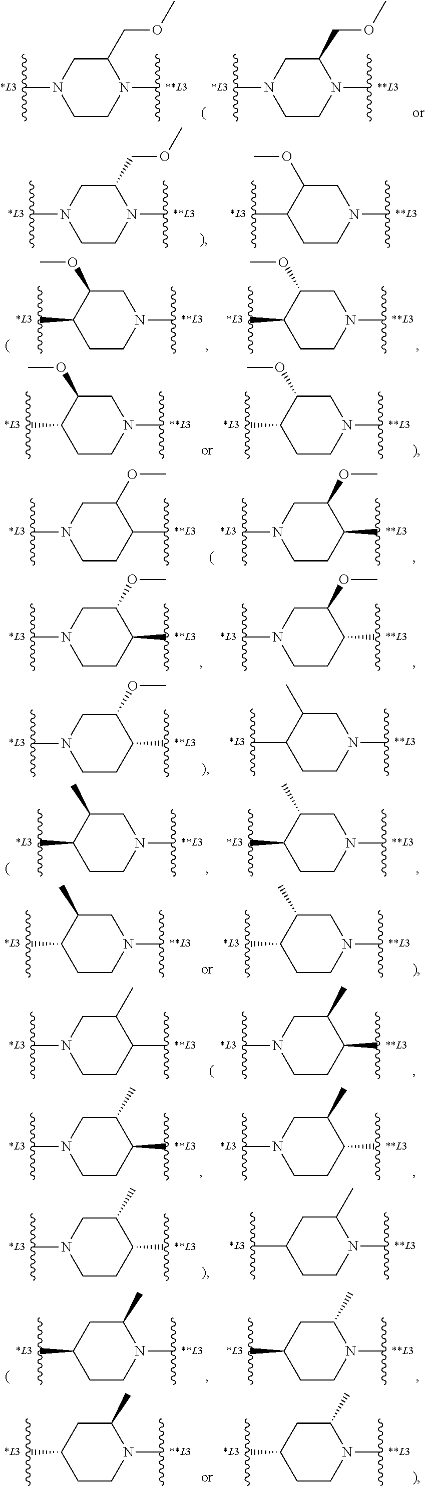

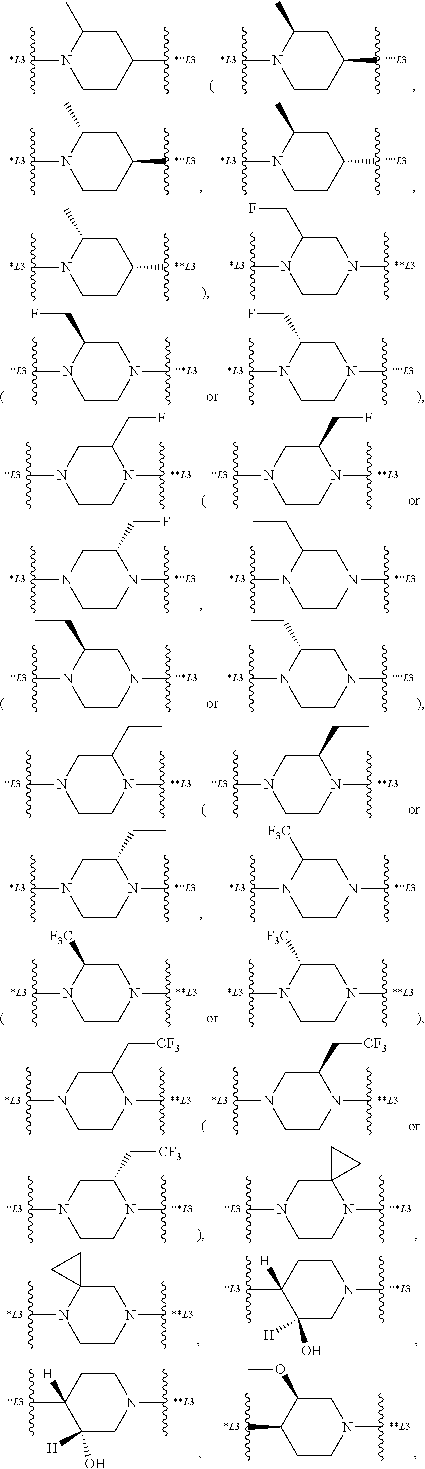

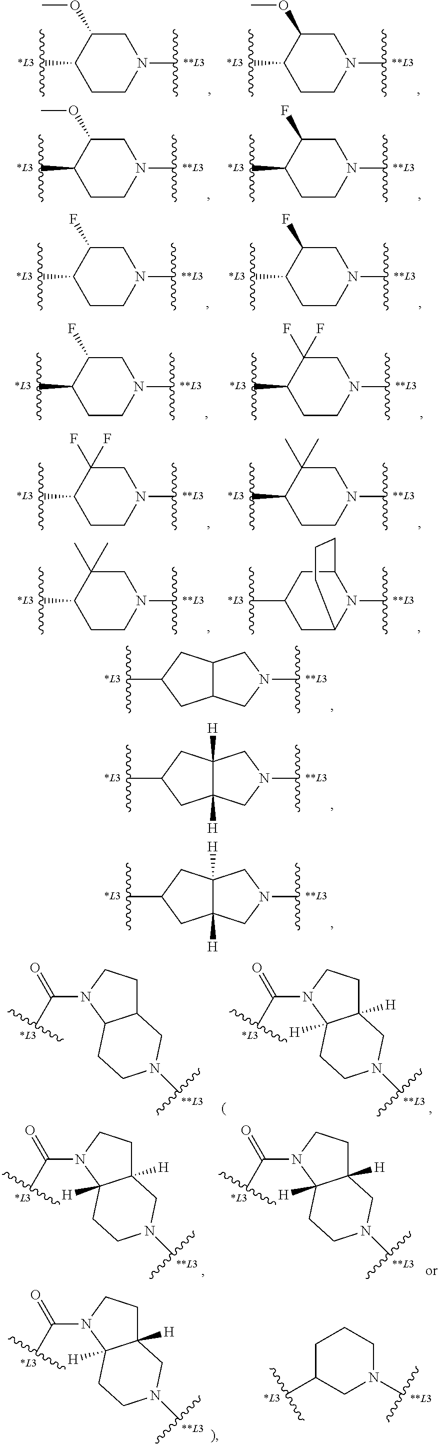

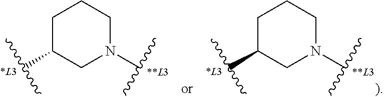 [SMILES: *[C@@H]1CCCN(*)C1 ; *[C@H]1CCCN(*)C1]

26. The compound of any one of the preceding claims, wherein 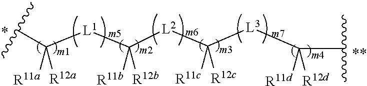
moiety is selected from

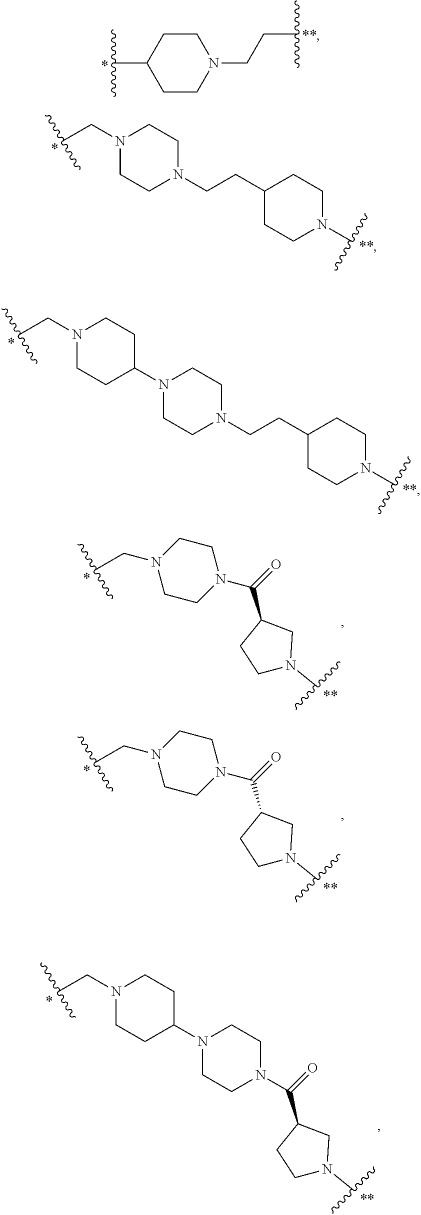

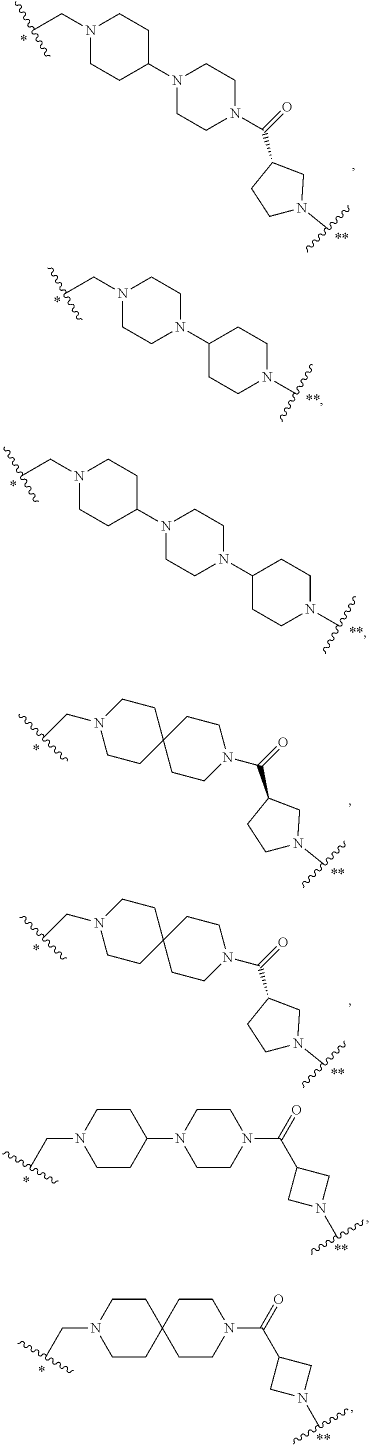

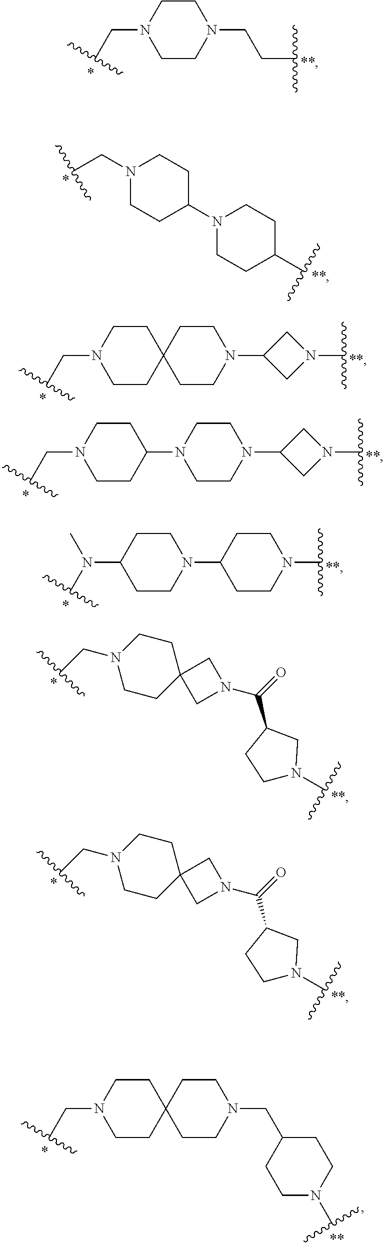

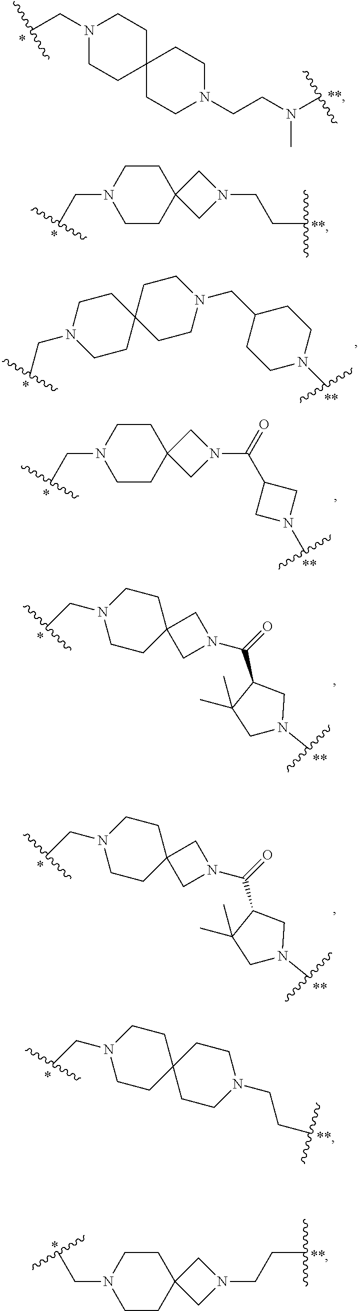

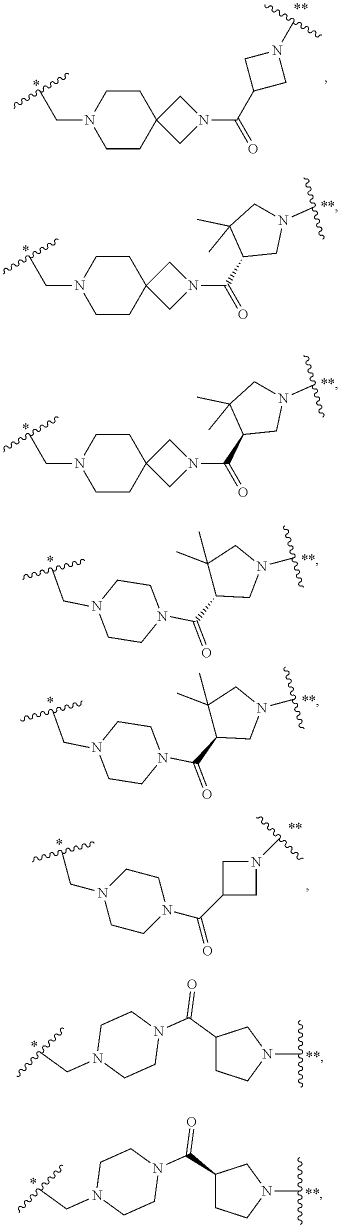

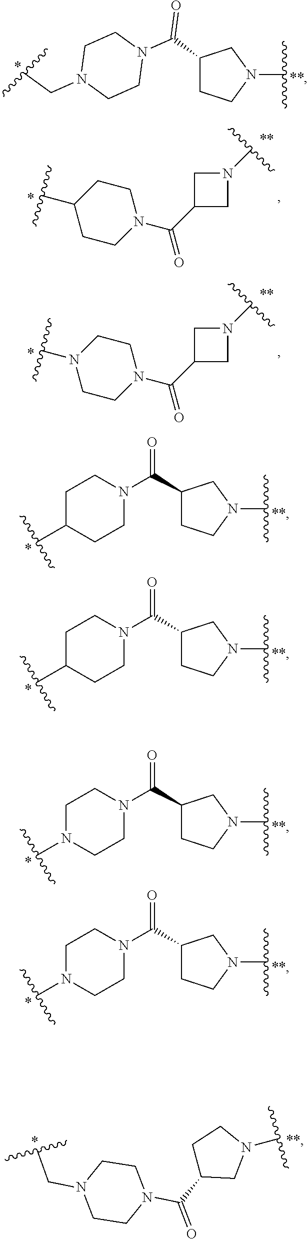

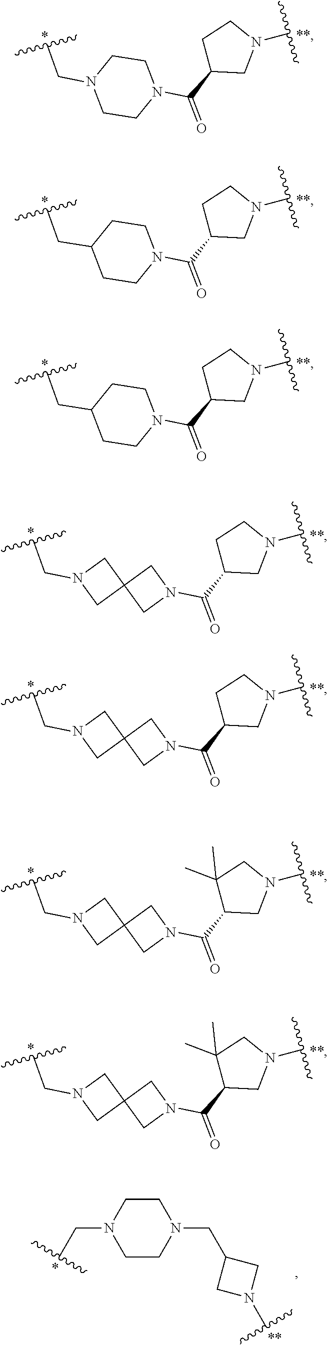

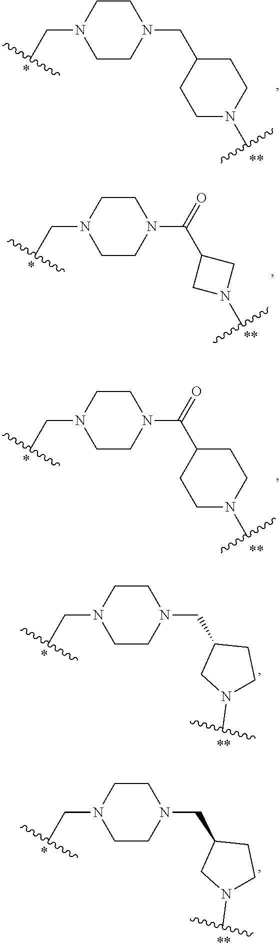 [SMILES: *CN1CCN(CC2CCN(*)CC2)CC1 ; *CN1CCN(C(=O)C2CN(*)C2)CC1 ; *CN1CCN(C(=O)C2CCN(*)CC2)CC1 ; *CN1CCN(C[C@@H]2CCN(*)C2)CC1 ; *CN1CCN(C[C@H]2CCN(*)C2)CC1]

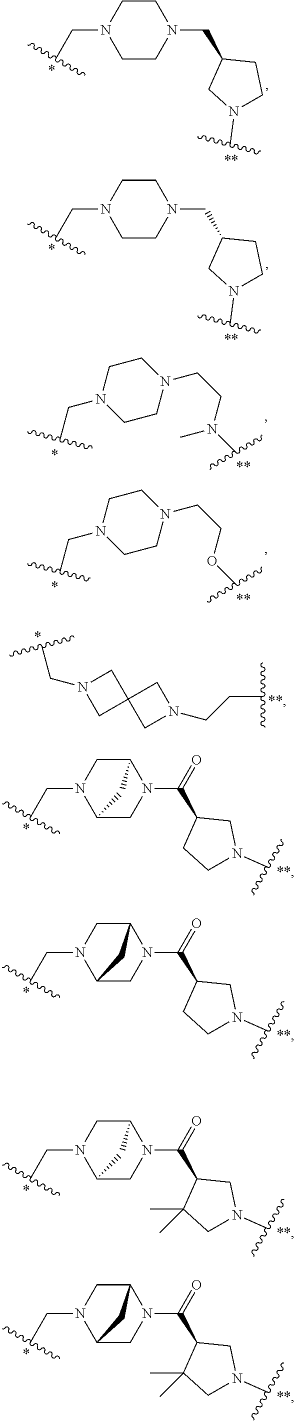

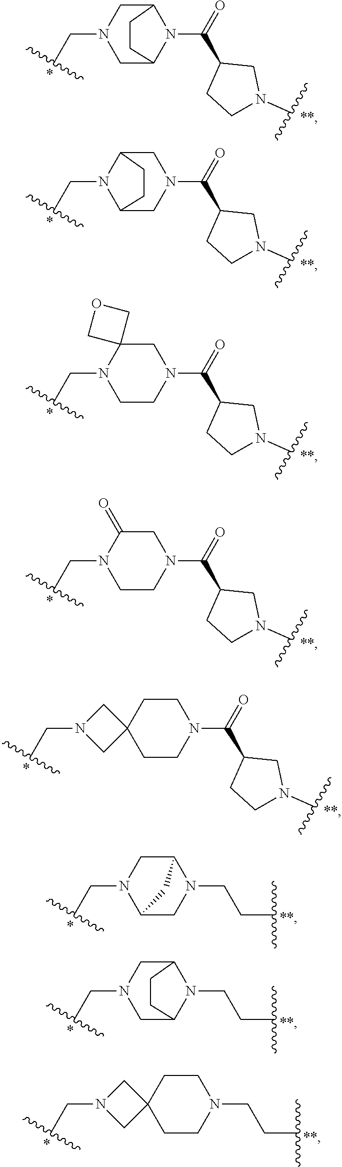

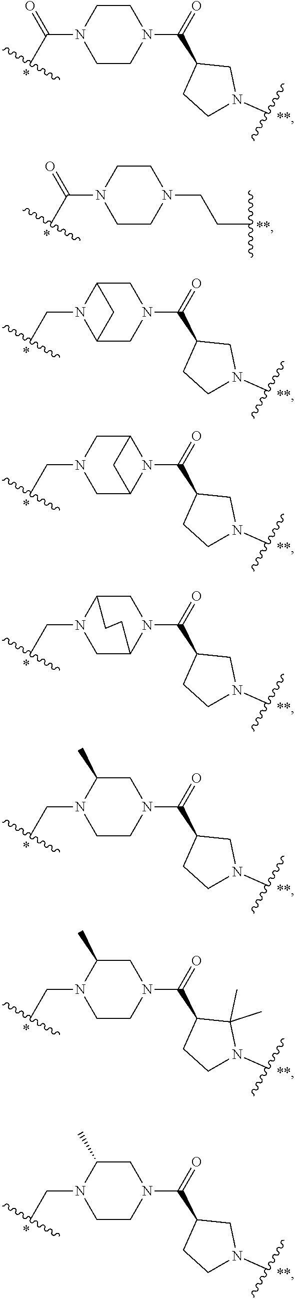

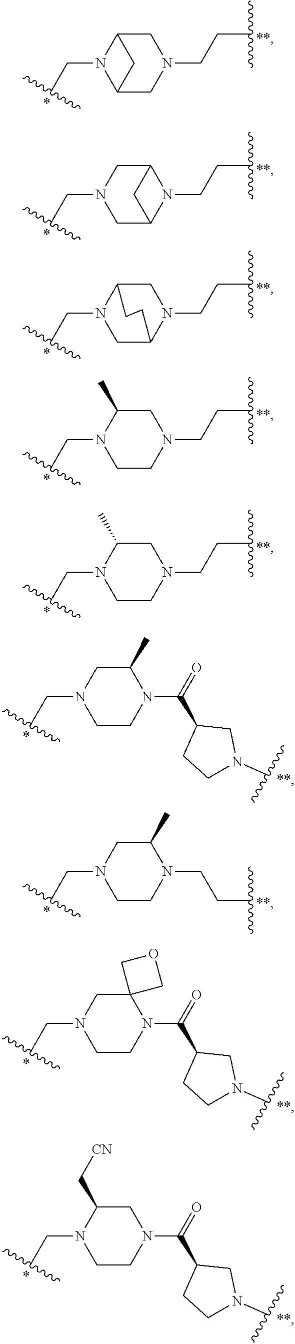

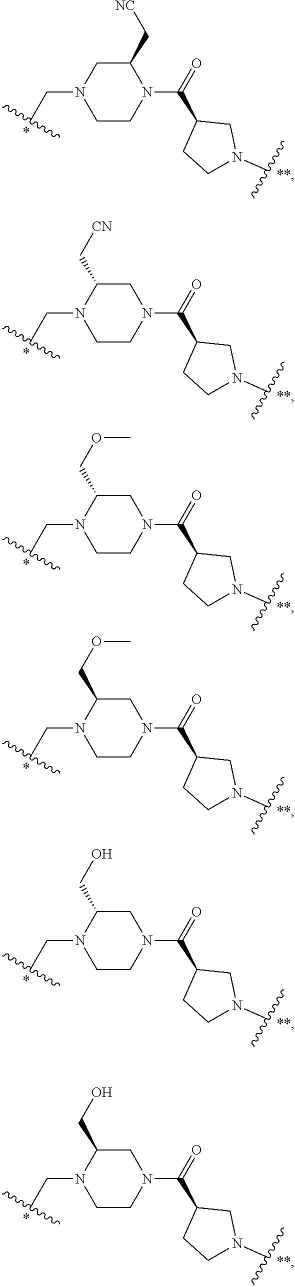 [SMILES: *CN1CCN(C(=O)[C@@H]2CCN(*)C2)[C@H](CC#N)C1 ; *CN1CCN(C(=O)[C@@H]2CCN(*)C2)C[C@H]1CC#N ; *CN1CCN(C(=O)[C@@H]2CCN(*)C2)C[C@H]1COC ; *CN1CCN(C(=O)[C@@H]2CCN(*)C2)C[C@@H]1COC ; *CN1CCN(C(=O)[C@@H]2CCN(*)C2)C[C@H]1CO ; *CN1CCN(C(=O)[C@@H]2CCN(*)C2)C[C@@H]1CO]

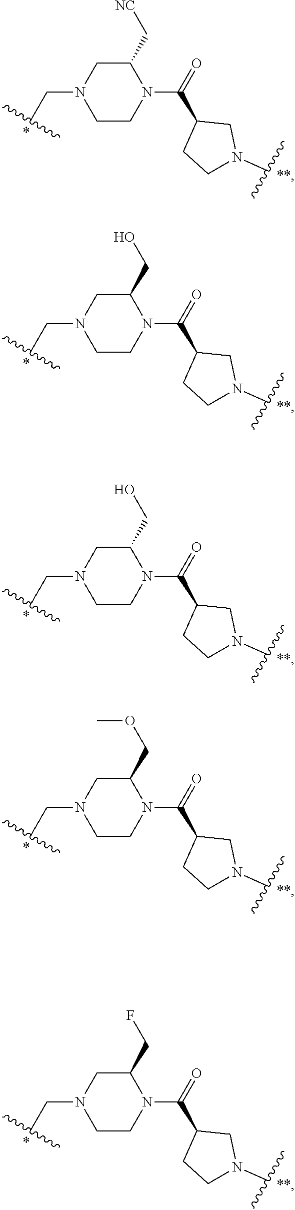 [SMILES: *CN1CCN(C(=O)[C@@H]2CCN(*)C2)[C@@H](CC#N)C1 ; *CN1CCN(C(=O)[C@@H]2CCN(*)C2)[C@H](CO)C1 ; *CN1CCN(C(=O)[C@@H]2CCN(*)C2)[C@@H](CO)C1 ; *CN1CCN(C(=O)[C@@H]2CCN(*)C2)[C@H](COC)C1 ; *CN1CCN(C(=O)[C@@H]2CCN(*)C2)[C@H](CF)C1]

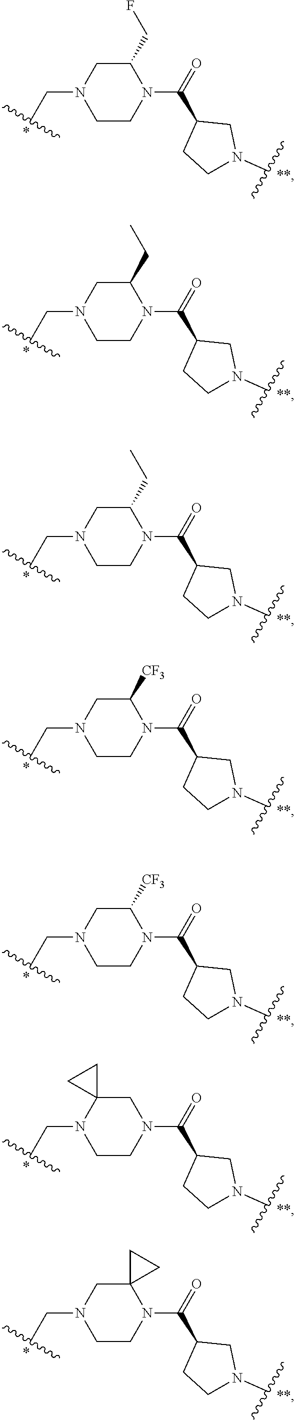

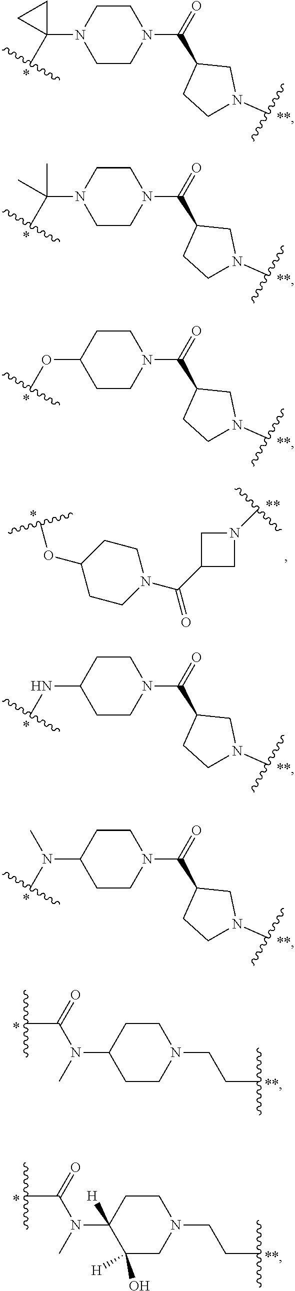

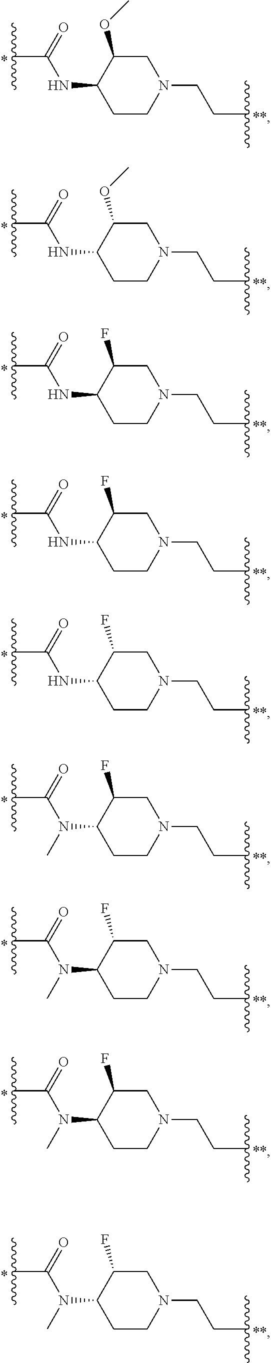

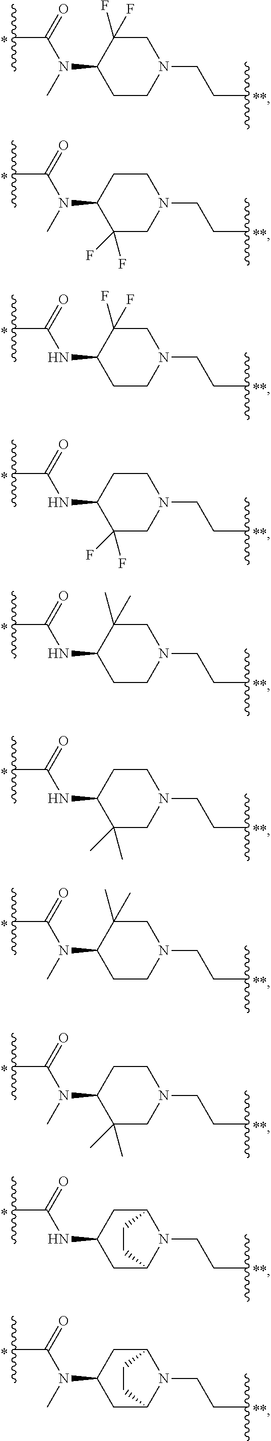

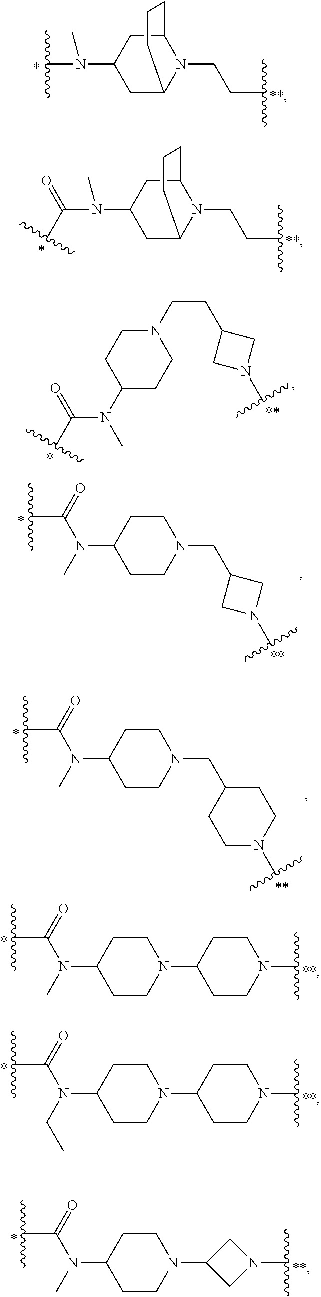

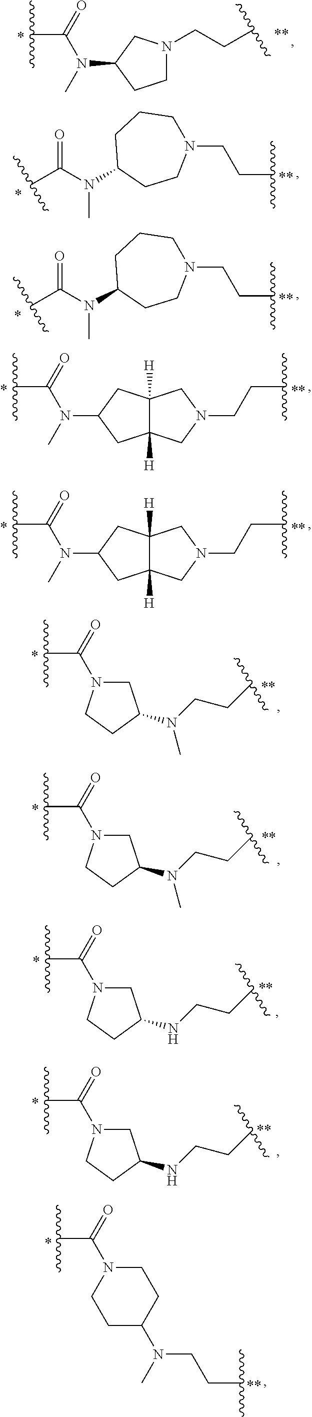

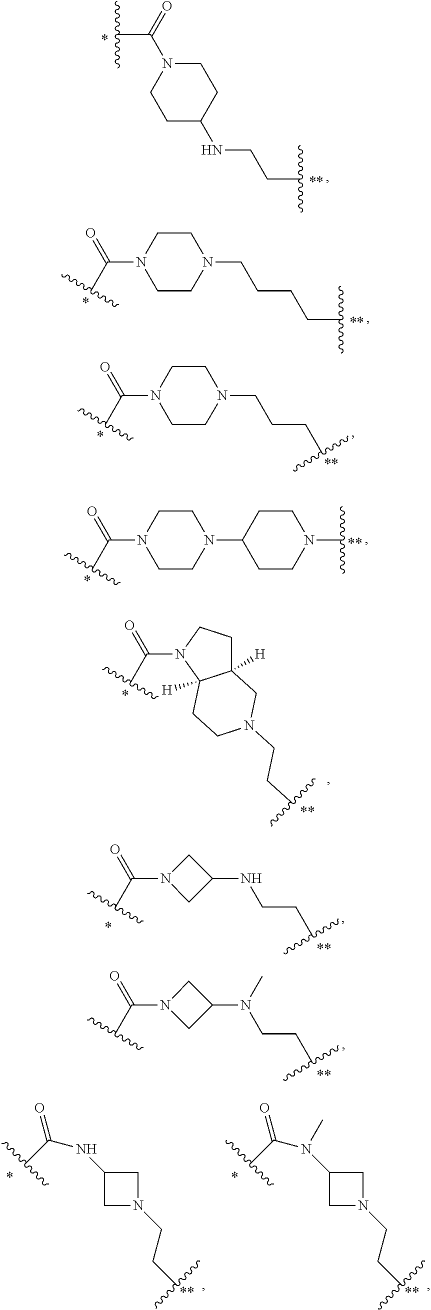

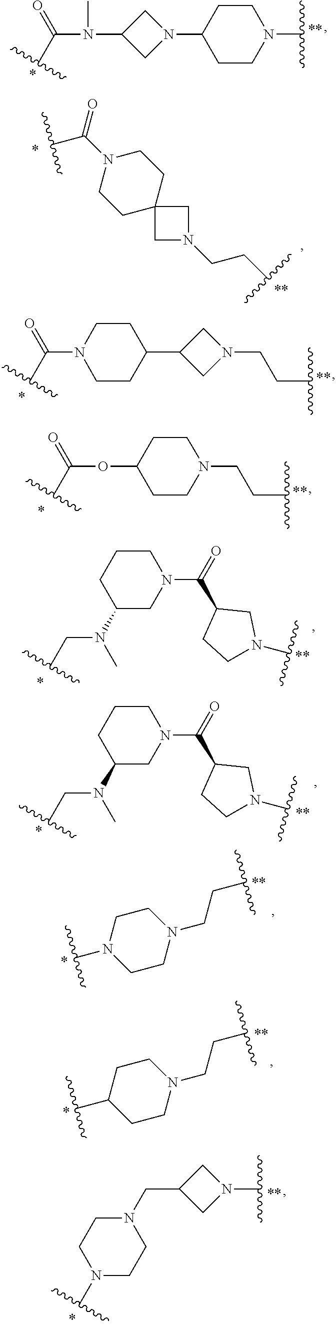

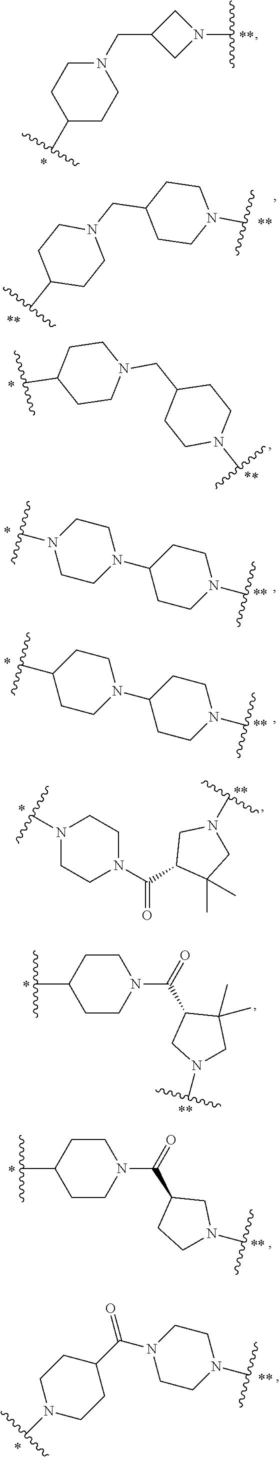

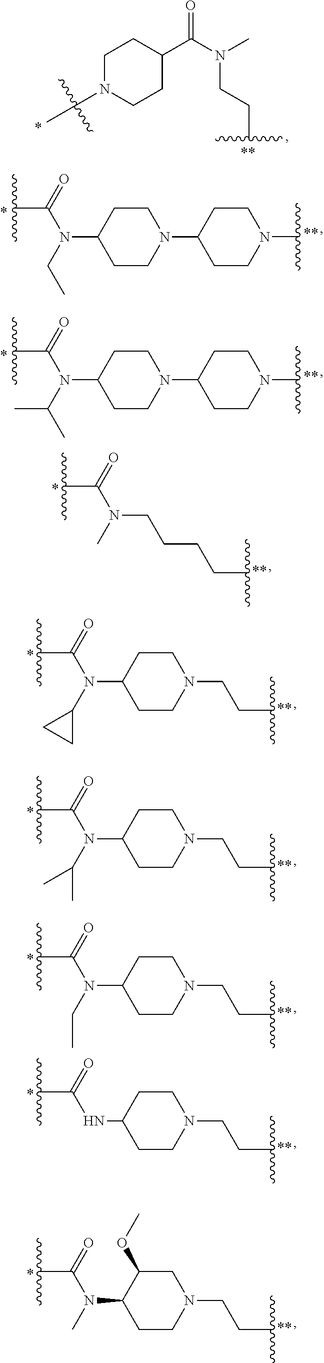

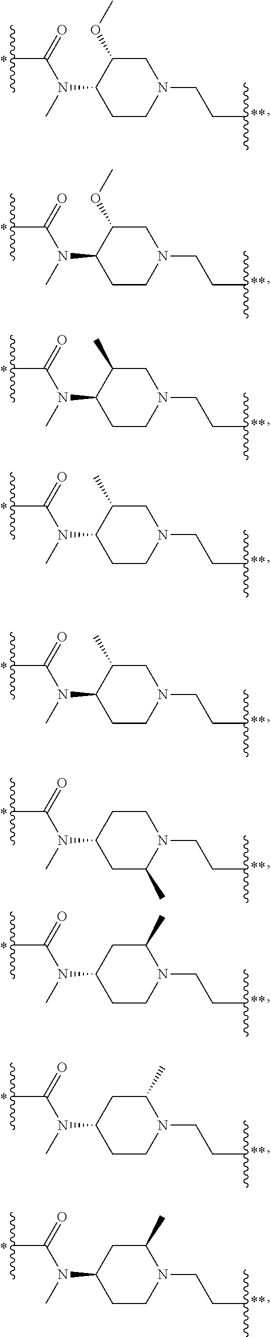

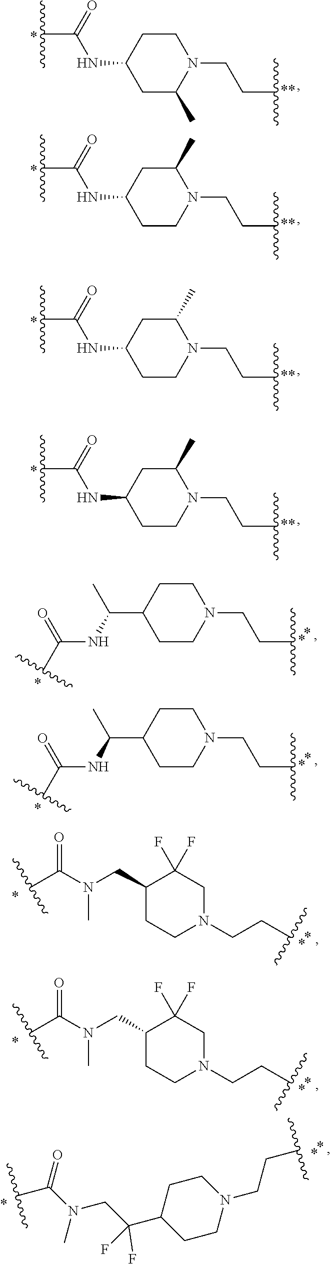

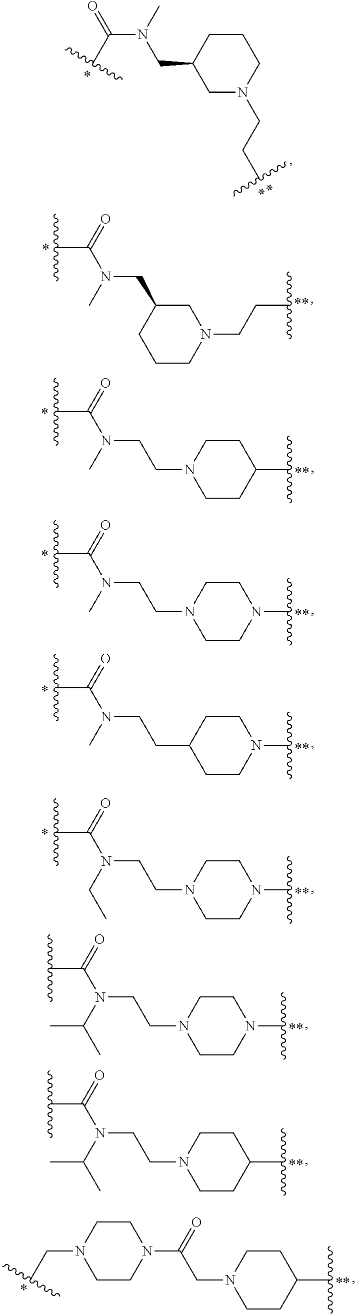

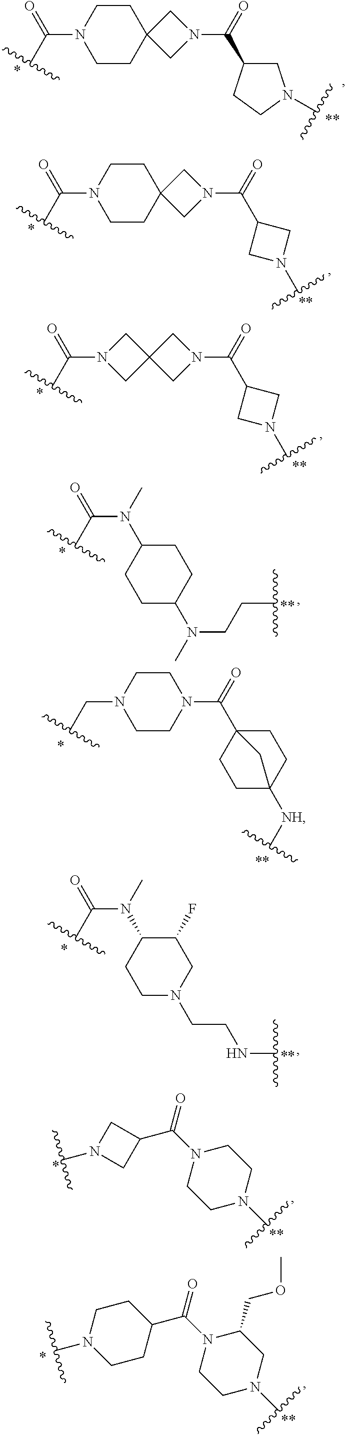

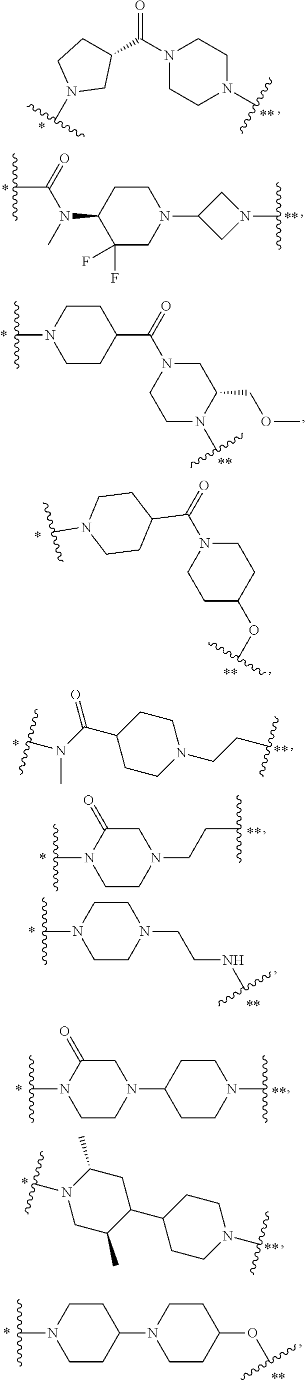

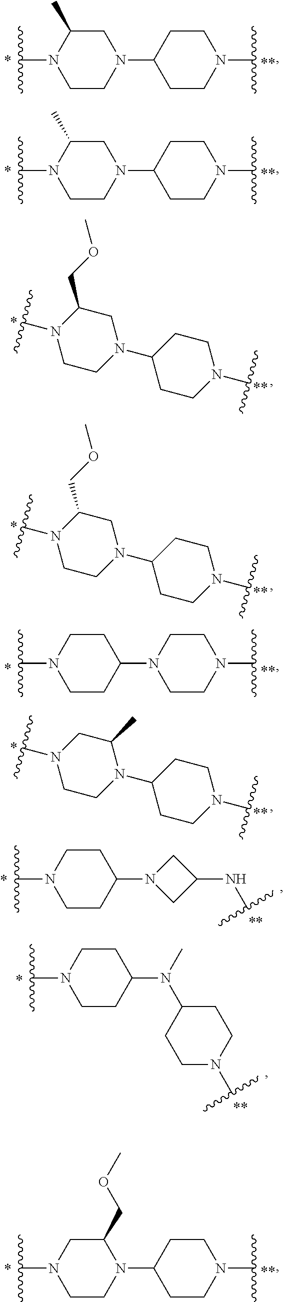

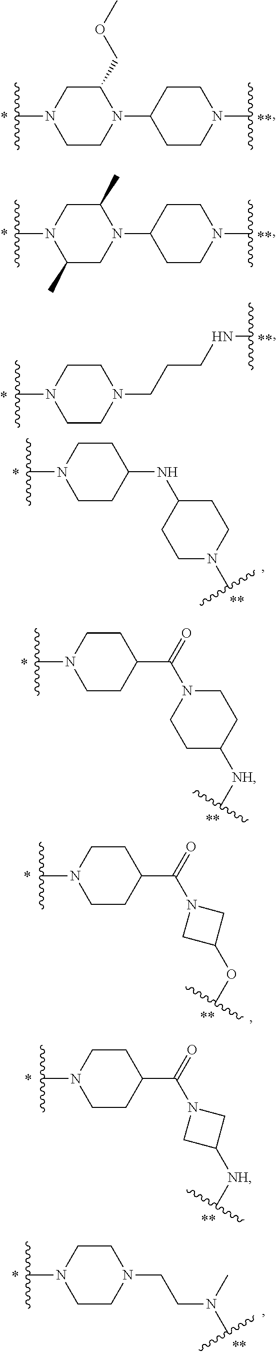

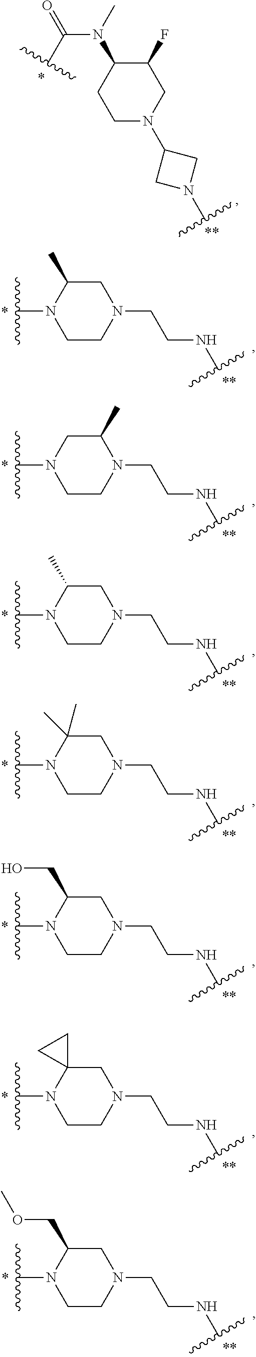

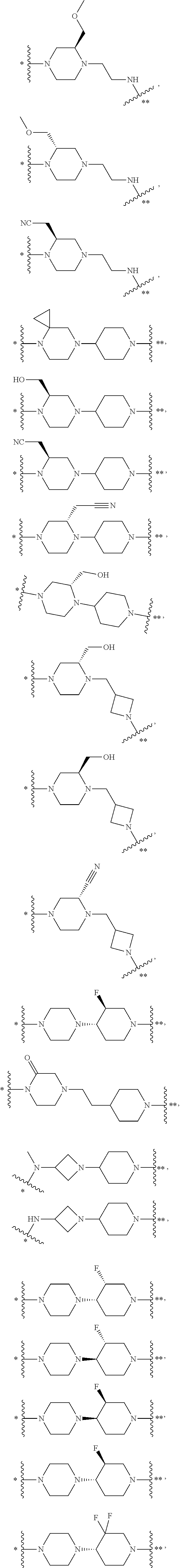

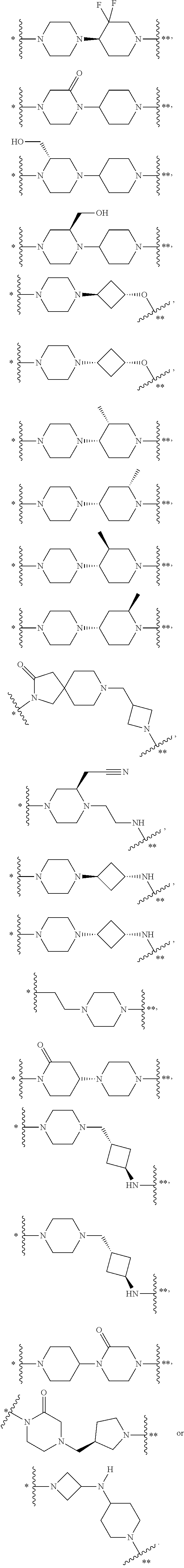

27. The compound of any one of the preceding claims, wherein L⁴ is independently selected from a single bond, —O—, —NR—, —(CR^{a}R^{b})ₙ₈—, —O(CR^{a}R^{b})ₙ₈—, —NR^{a}(CR^{a}R^{b})ₙ₈— or —C(O)—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

28. The compound of any one of the preceding claims, wherein L⁴ is independently selected from a single bond.

29. The compound of any one of the preceding claims, wherein X⁷ is independently selected from —CR^{a}, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

30. The compound of any one of the preceding claims, wherein X⁷ is independently selected from —CH, —C(CH₃), or N; preferably X⁷ is independently selected from —CH.

31. The compound of any one of the preceding claims, wherein X⁸ is independently selected from —NR^{a}—, —O—, —S— and —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

32. The compound of any one of the preceding claims, wherein X⁸ is independently selected from —NH— and —CH₂—; preferably X⁸ is independently selected from —CH₂—.

33. The compound of any one of the preceding claims, wherein 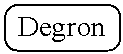
is selected from 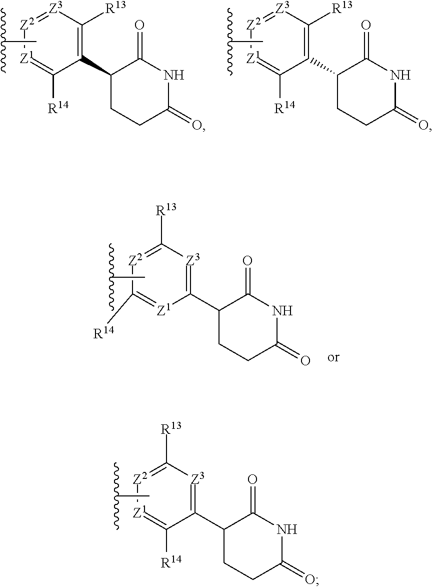
preferably, 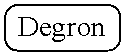
is selected from

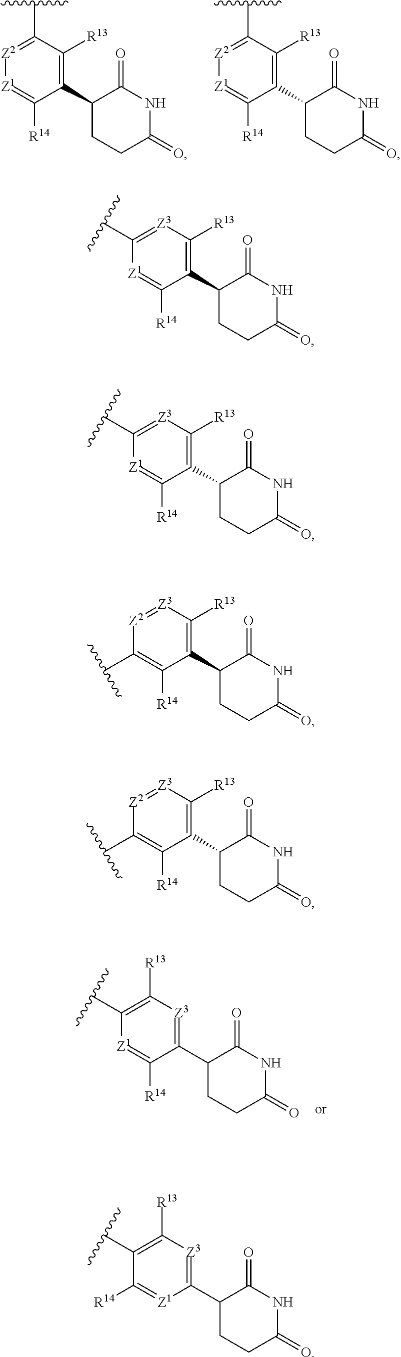

34. The compound of any one of the preceding claims, wherein at most one of Z¹, Z² and Z³ is N.

35. The compound of any one of the preceding claims, wherein Z¹, Z² and Z³ are each independently CR^{z}.

36. The compound of any one of the preceding claims, wherein R^{z}, at each occurrence, is independently selected from hydrogen, —F, —Cl, —Br, —I, methyl, ethyl, propyl, butyl, pentyl, hexyl, heptyl, octyl, —NR^{Za}R^{Zb}—, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc}; R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl.

37. The compound of any one of the preceding claims, wherein R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

38. The compound of any one of the preceding claims, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR¹³, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

39. The compound of any one of the preceding claims, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

40. The compound of any one of the preceding claims, wherein 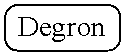
is

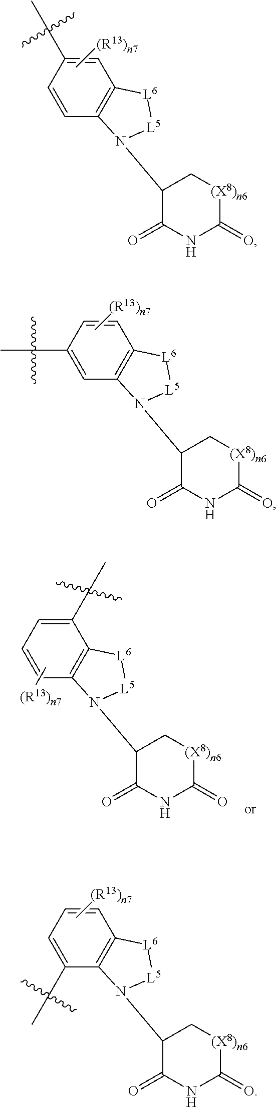

41. The compound of any one of the preceding claims, wherein L⁵ and L⁶ are each independently selected from a single bond, —O—, —NR^{a}—, —(CR^{a}R^{b})ₙ₈—, —O(CR^{a}R^{b})ⁿ⁸—, —NR^{a}(CR^{a}R^{b})ₙ₈— or —C(O)—; X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

42. The compound of any one of the preceding claims, wherein L⁵ and L⁶ are each independently a single bond, 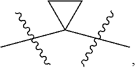 [SMILES: *C1(*)CC1]
—O—, —NH—, —NMe-, —N(CH₂CH₃)—, —CH₂—, —CHF—, —CF₂—, —C(CH₃)₂— or —CO— (preferably L⁵ is —CO— or —CH₂—, and L⁶ is 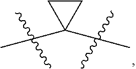 [SMILES: *C1(*)CC1]
—O—, —NH—, —NMe-, -N(CH₂CH₃)—, —CH₂—, —CHF—, —CF₂—, —C(CH₃)₂— or —CO—);
X⁸ is CH₂; and
n6 is 0 or 1.

43. The compound of any one of the preceding claims, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13c}O₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

44. The compound of any one of the preceding claims, wherein R¹³ is independently selected from hydrogen, F, Cl, Br, I, CN, —C₁-C₈alkyl, or —C₁-C₈alkoxy; preferably R¹³ is independently selected from hydrogen, F, Cl, Br, I, CN, -Me, -Et, —C₃H₇, —C₄H₉, —OMe, -OEt, —OC₃H₇ or —OC₄H₉; n7 is 0, 1 or 2.

45. The compound of any one of the preceding claims, wherein 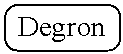
is

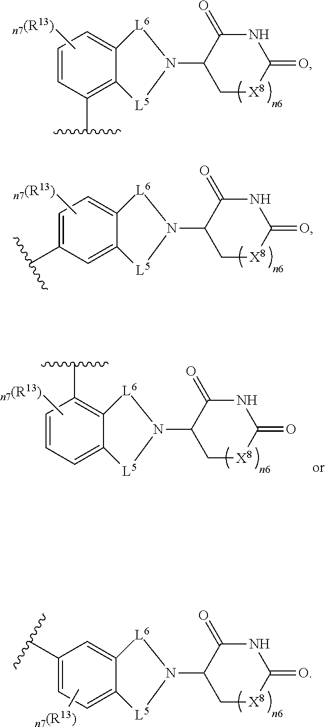

46. The compound of any one of the preceding claims, wherein L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

47. The compound of any one of the preceding claims, wherein L¹ and L⁶ are each independently a single bond, 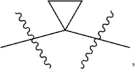 [SMILES: *C1(*)CC1]
—O—, —NH—, —NMe-, -N(CH₂CH₃)—, —CH₂—, —CHF—, —CF₂—, —C(CH₃)₂— or —CO-(preferably L⁵ and L⁶ are each independently —CO— or —CH₂-):
X⁵ is CH₂; and
n6 is 0 or 1.

48. The compound of any one of the preceding claims, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}, —SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocycyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}OR^{13d}, —NR^{13c}O₂R^{13d}, or —NR^{13c}SO₂R^{13d}; at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

49. The compound of any one of the preceding claims, wherein R¹³ is independently selected from hydrogen, F, Cl, Br, I, CN, —C₁-C₈alkyl, or —C₁-C₈alkoxy: preferably R¹³ is independently selected from hydrogen, F, Cl, Br, I, CN, -Me, -Et, —C₃H₇, —C₄H₉, —OMe, -OEt, —OC₃H₇ or —OC₄H₉; n7 is 0, 1 or 2.

50. The compound of any one of the preceding claims, wherein 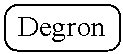
is

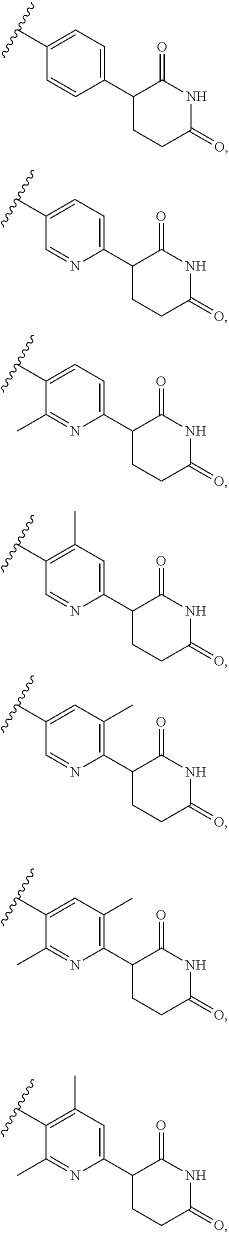

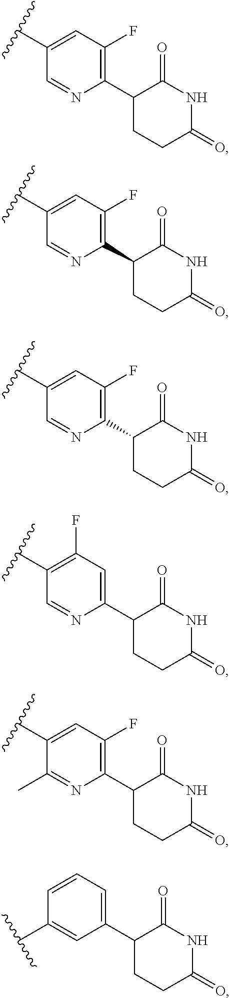 [SMILES: *c1cnc(C2CCC(=O)NC2=O)c(F)c1 ; *c1cnc([C@@H]2CCC(=O)NC2=O)c(F)c1 ; *c1cnc([C@H]2CCC(=O)NC2=O)c(F)c1 ; *c1cnc(C2CCC(=O)NC2=O)cc1F ; *c1cc(F)c(C2CCC(=O)NC2=O)nc1C ; *c1cccc(C2CCC(=O)NC2=O)c1]

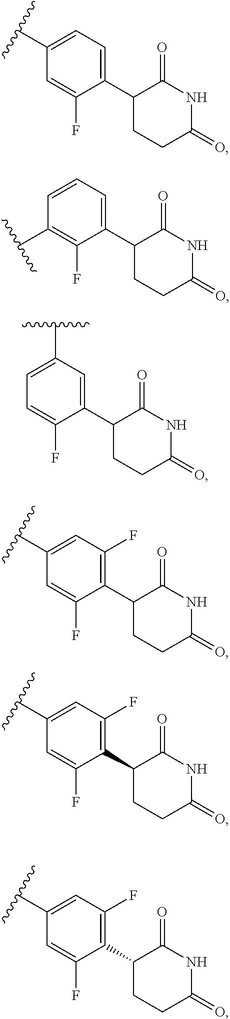 [SMILES: *c1ccc(C2CCC(=O)NC2=O)c(F)c1 ; *c1cccc(C2CCC(=O)NC2=O)c1F ; *c1ccc(F)c(C2CCC(=O)NC2=O)c1 ; *c1cc(F)c(C2CCC(=O)NC2=O)c(F)c1 ; *c1cc(F)c([C@@H]2CCC(=O)NC2=O)c(F)c1 ; *c1cc(F)c([C@H]2CCC(=O)NC2=O)c(F)c1]

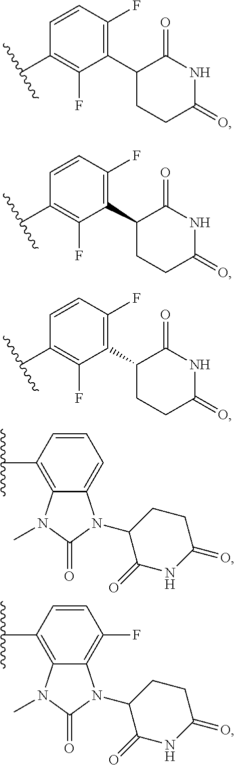

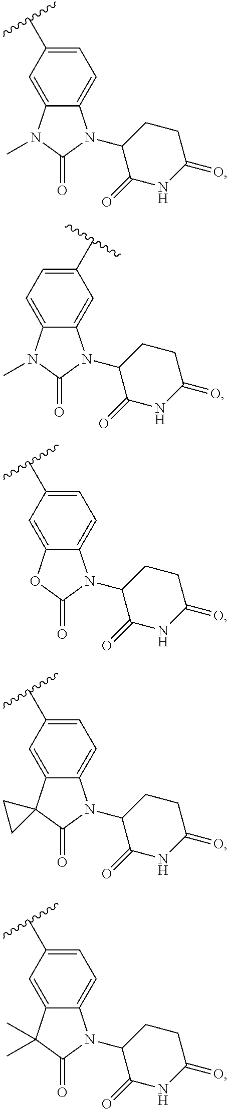

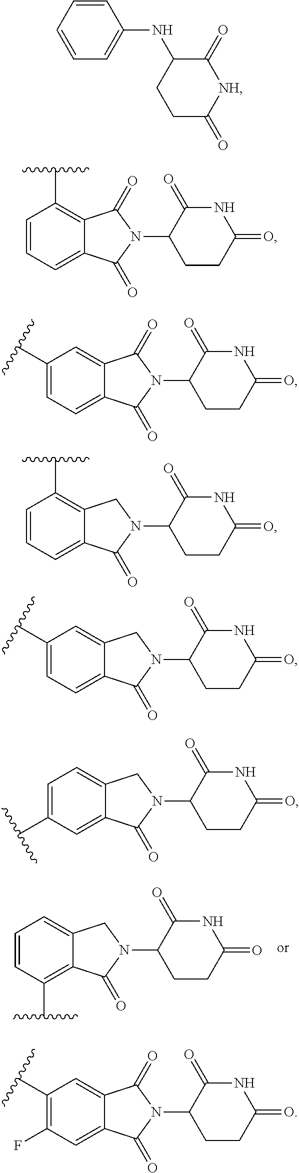

51. The compound of any one of the preceding claims, wherein the 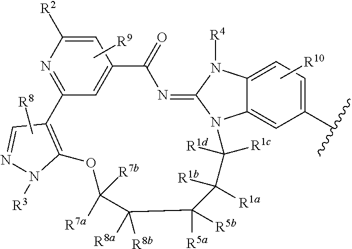
moiety is

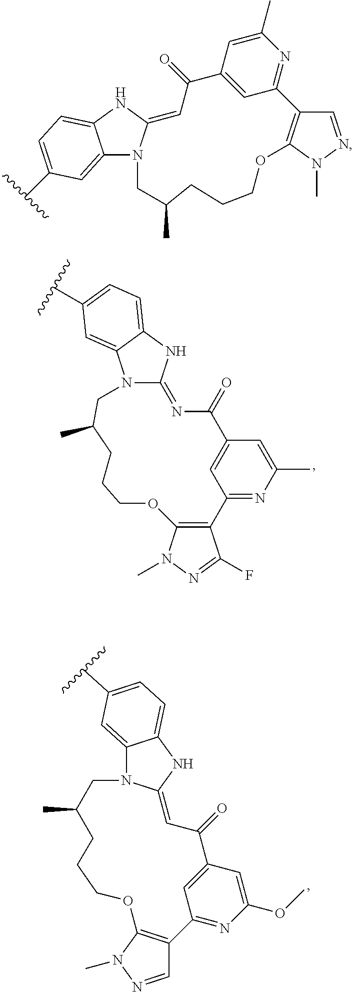

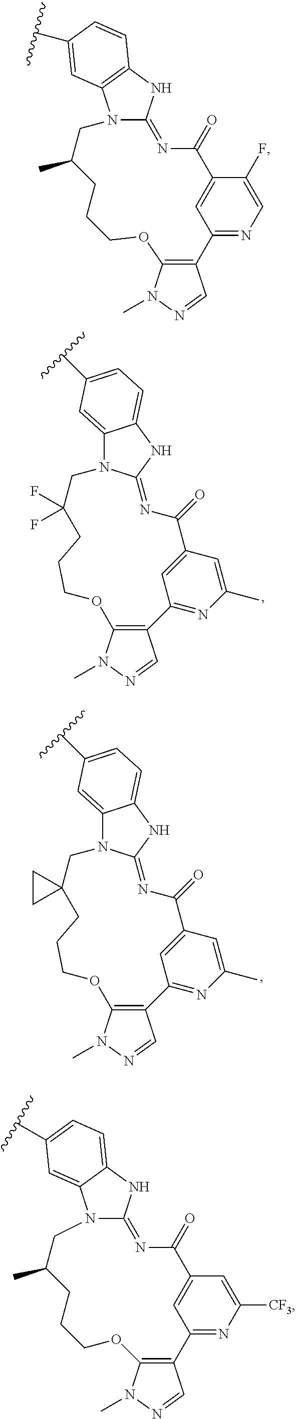

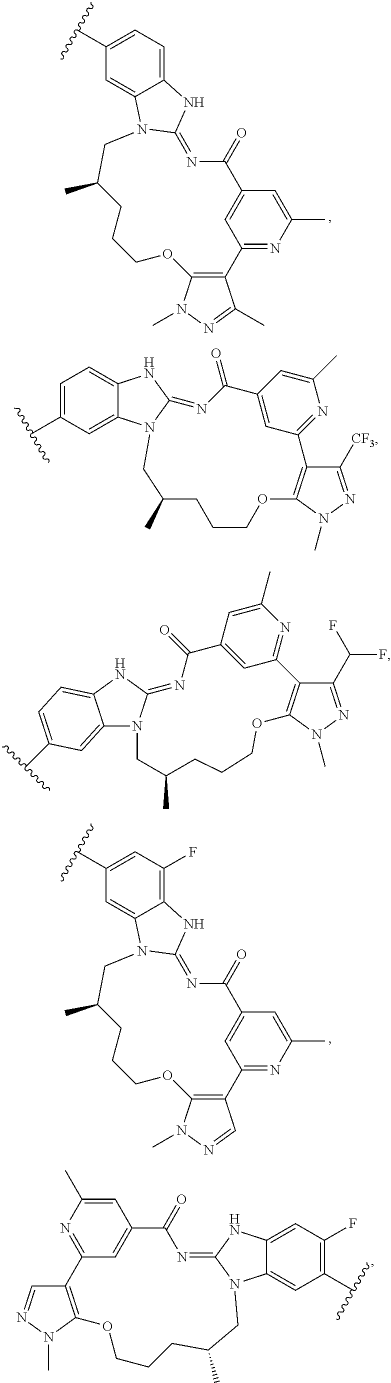

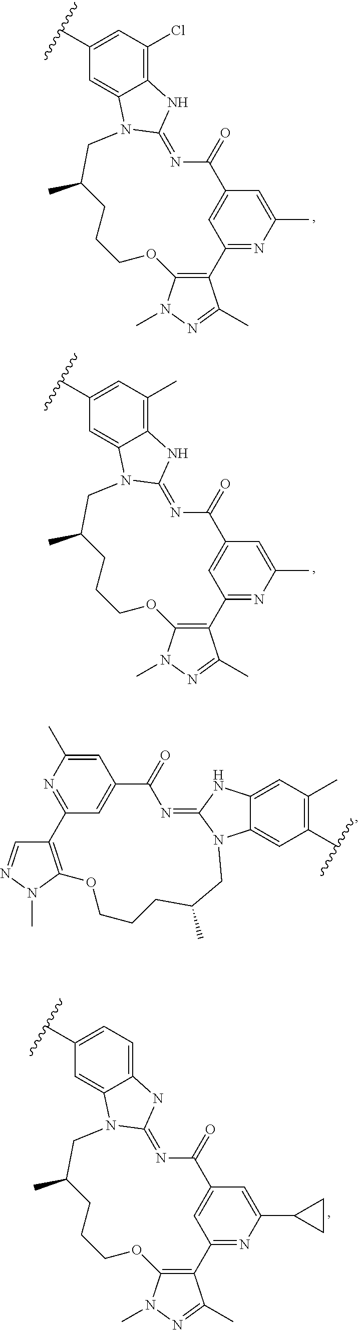

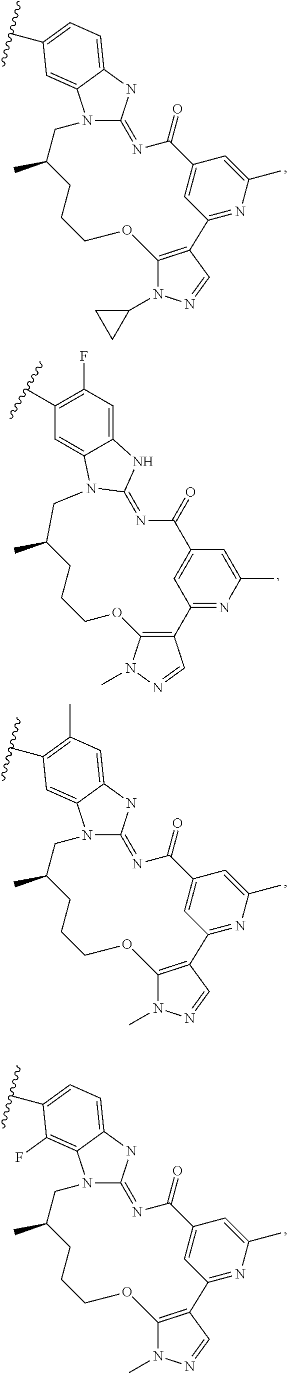

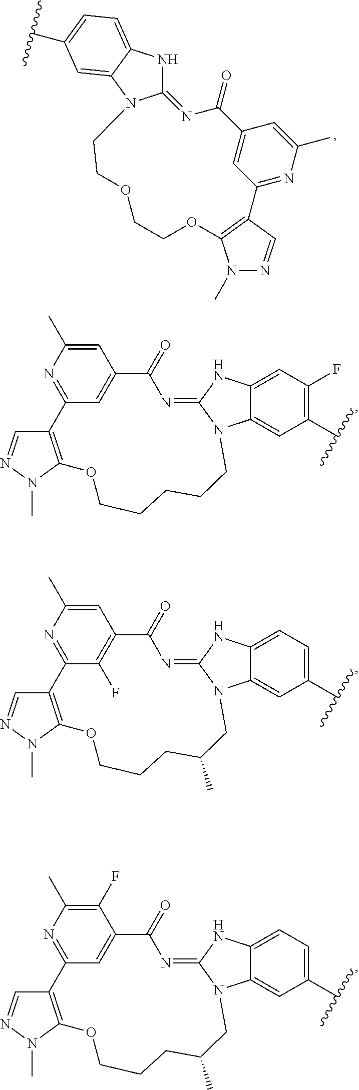 [SMILES: *c1ccc2c(c1)N1CCOCCOc3c(cnn3C)-c3cc(cc(C)n3)C(=O)/N=C/1N2 ; *c1cc2c(cc1F)N/C1=N\C(=O)c3cc(C)nc(c3)-c3cnn(C)c3OCCCCCN12 ; *c1ccc2c(c1)N1C[C@H](C)CCCOc3c(cnn3C)-c3nc(C)cc(c3F)C(=O)/N=C/1N2 ; *c1ccc2c(c1)N1C[C@H](C)CCCOc3c(cnn3C)-c3cc(c(F)c(C)n3)C(=O)/N=C/1N2]

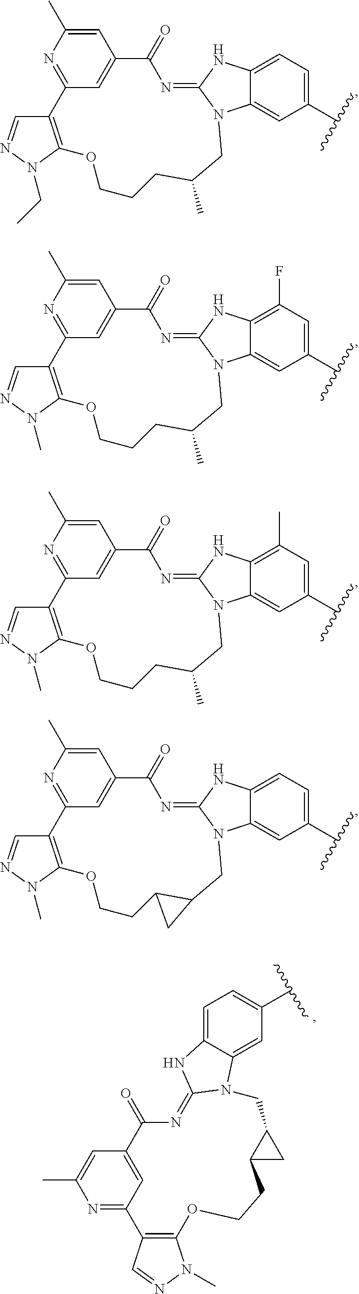

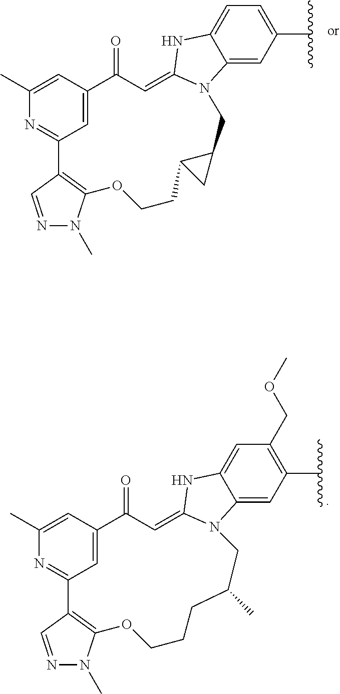 [SMILES: *c1ccc2c(c1)N1C[C@H]3C[C@@H]3CCOc3c(cnn3C)-c3cc(cc(C)n3)C(=O)/C=C/1N2 ; *c1cc2c(cc1COC)N/C1=C\C(=O)c3cc(C)nc(c3)-c3cnn(C)c3OCCC[C@@H](C)CN12]

52. The compound of any one of the preceding claims is selected from

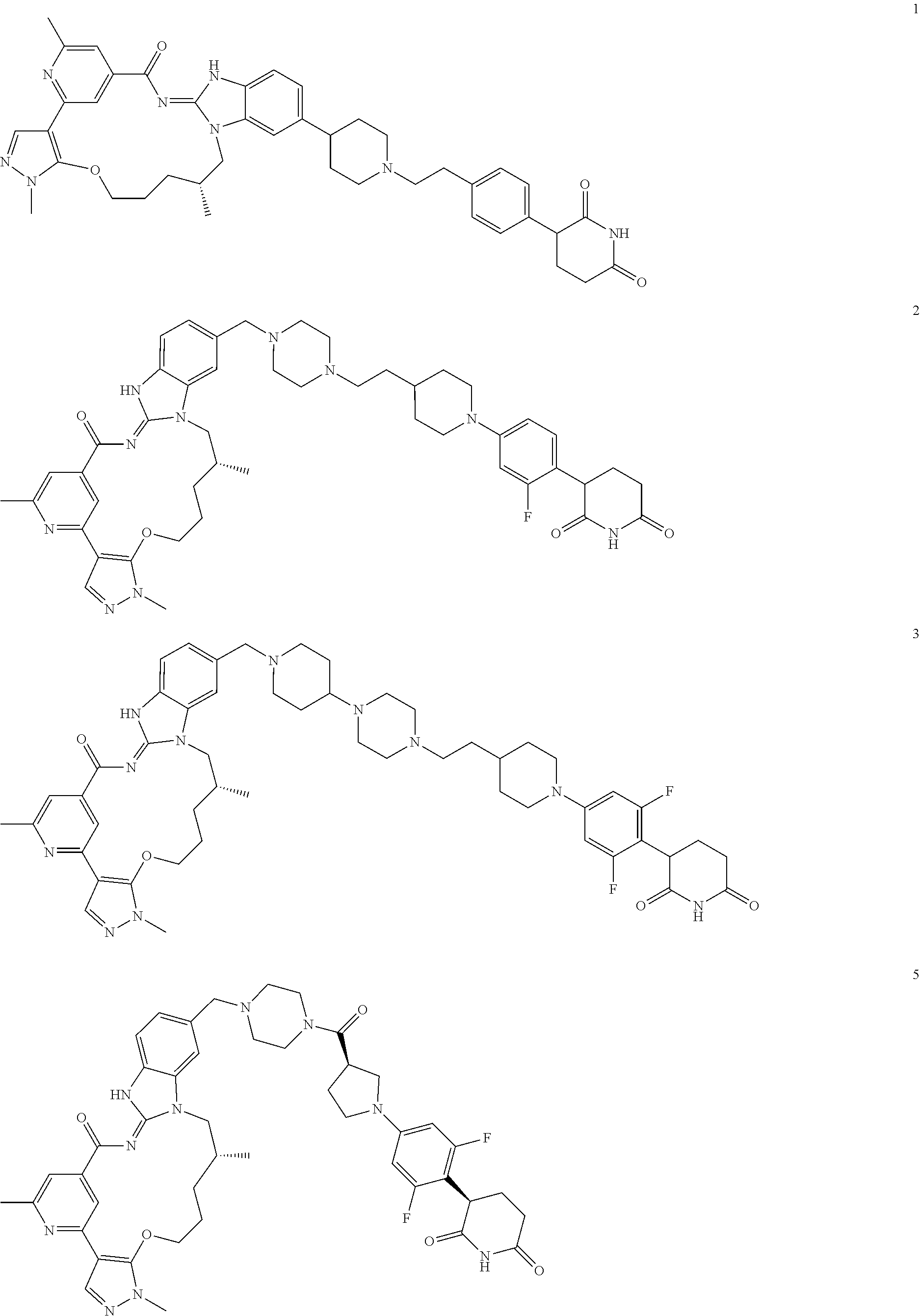

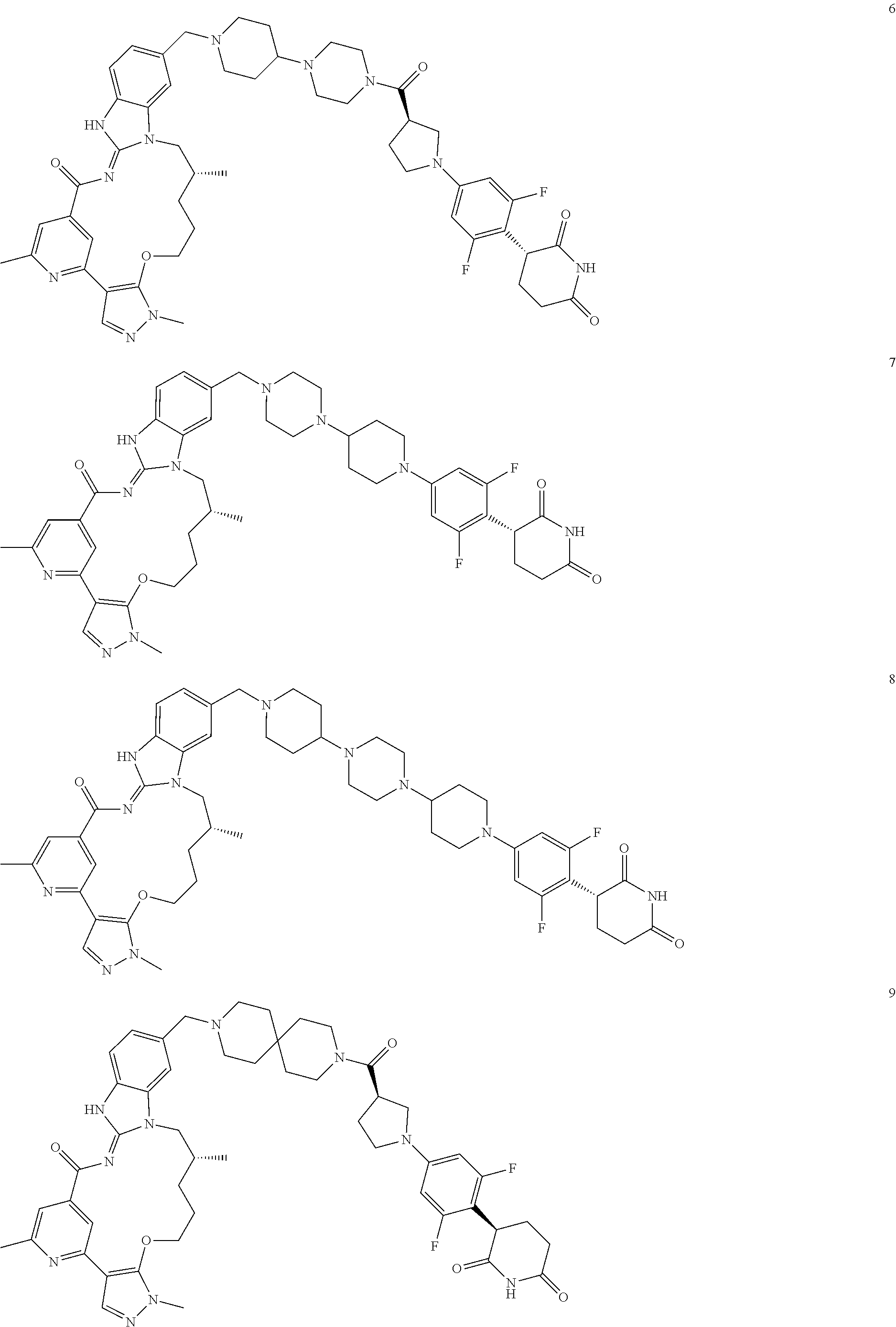

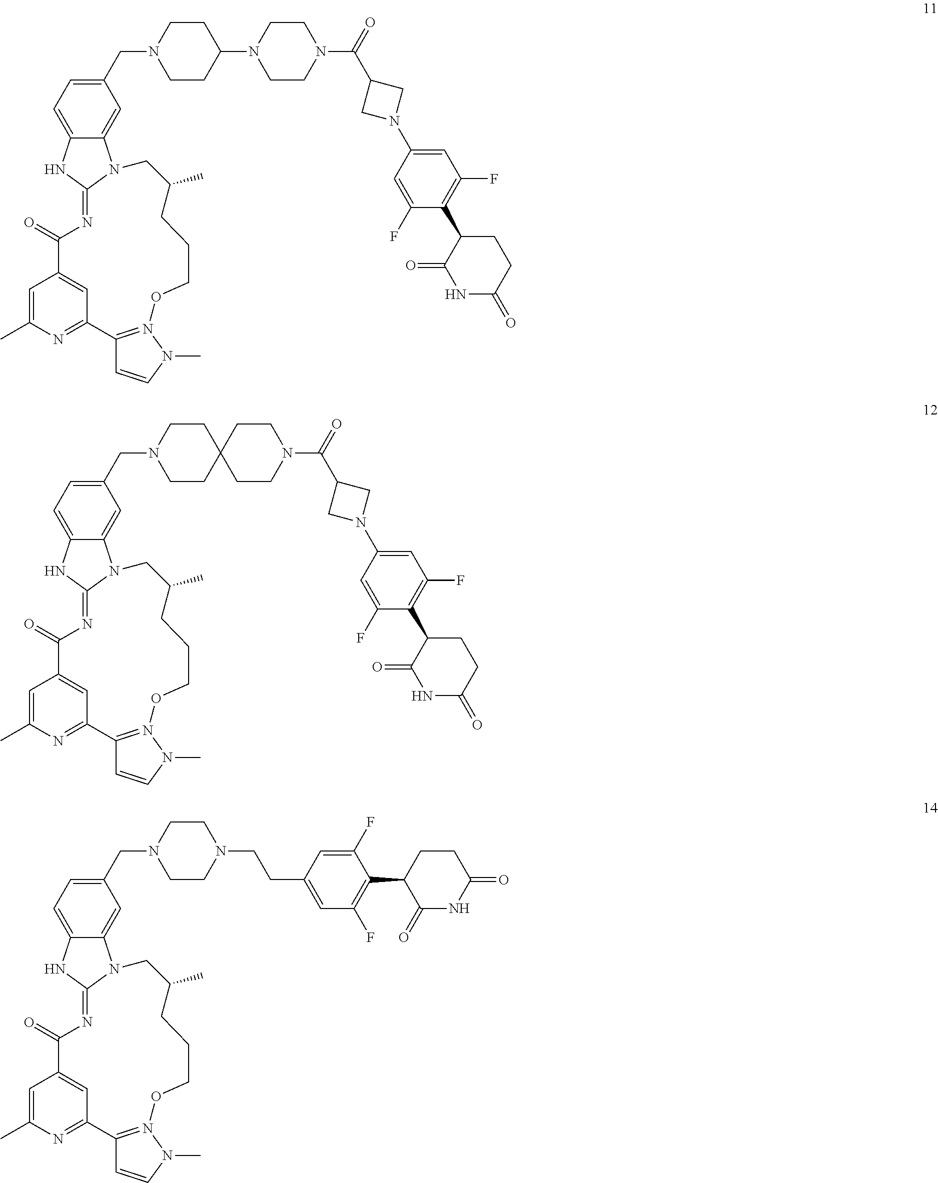

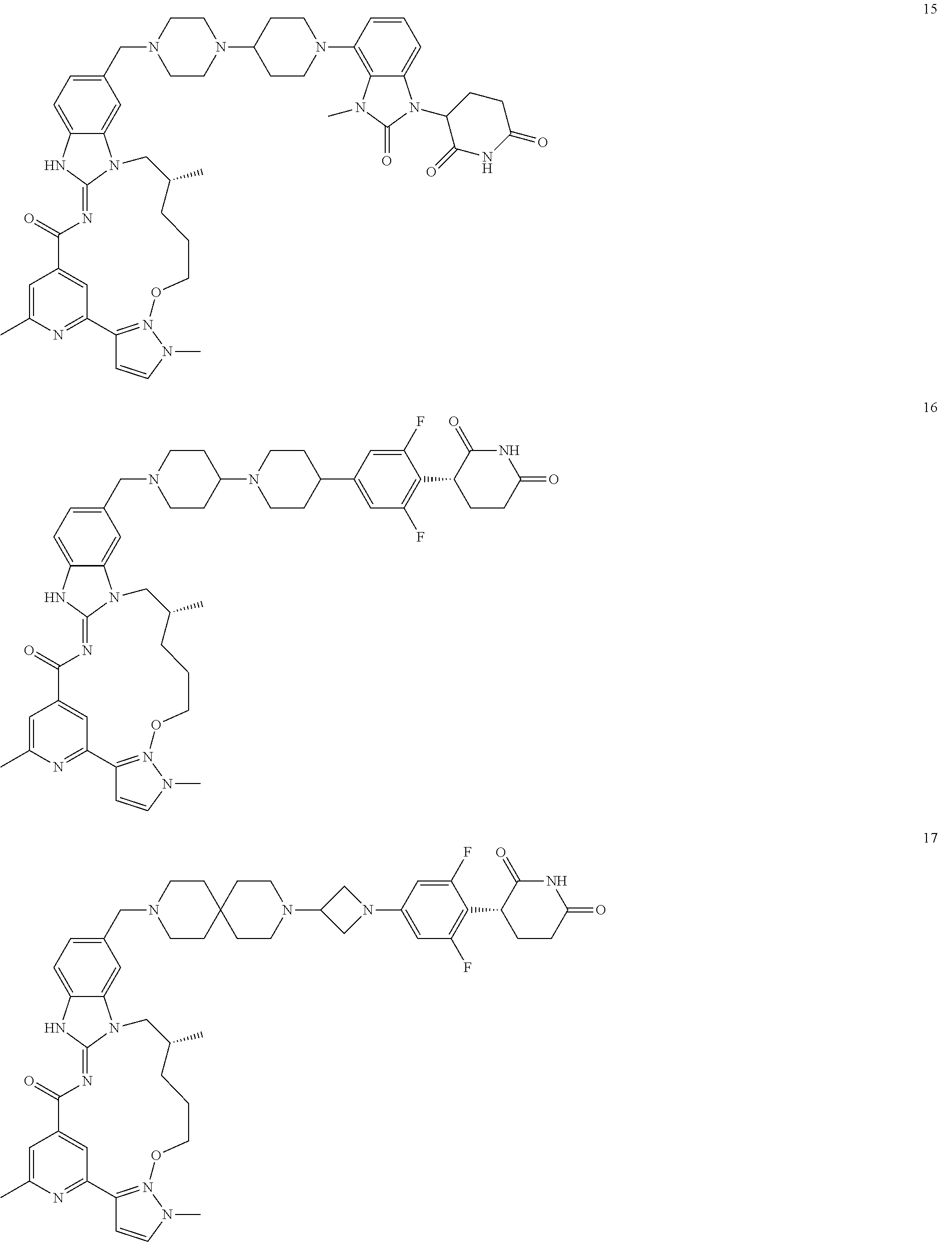

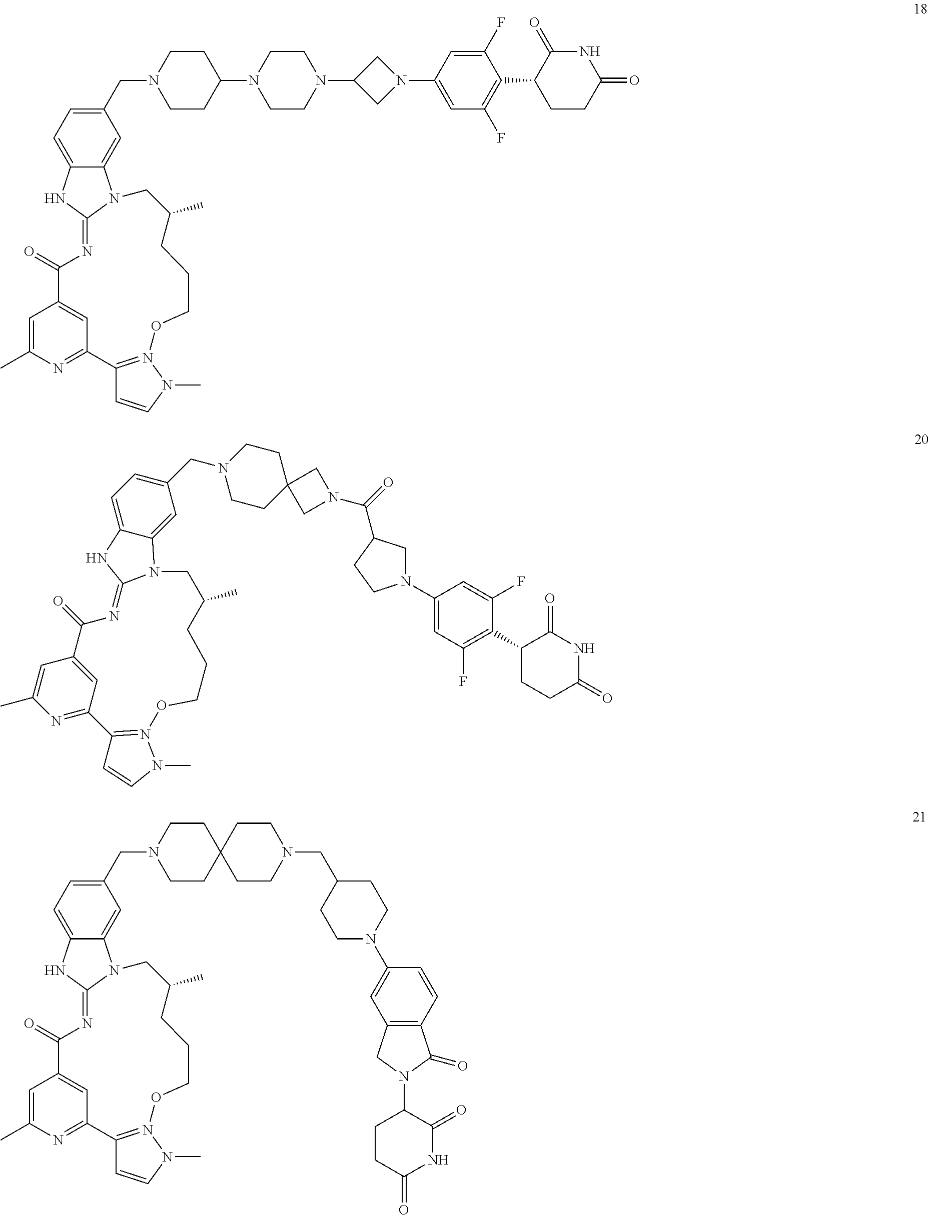

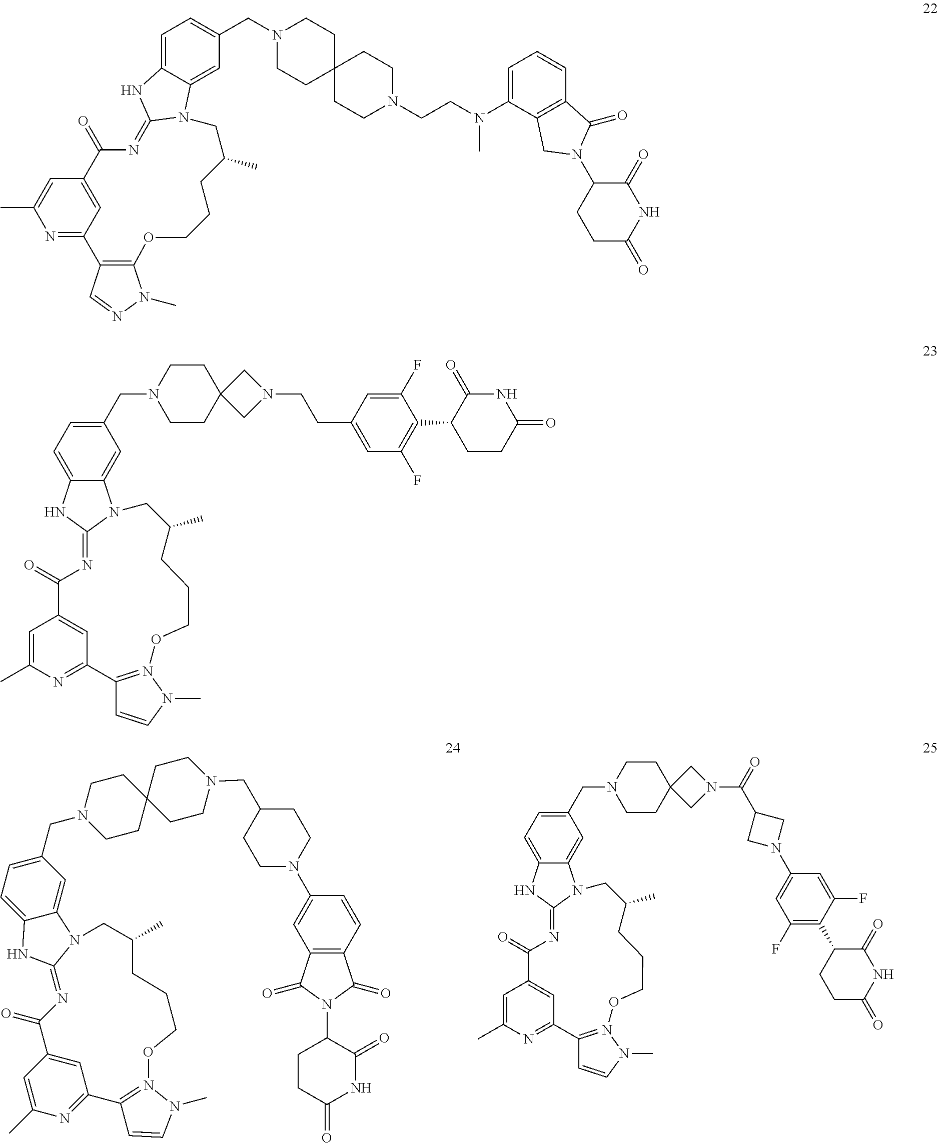

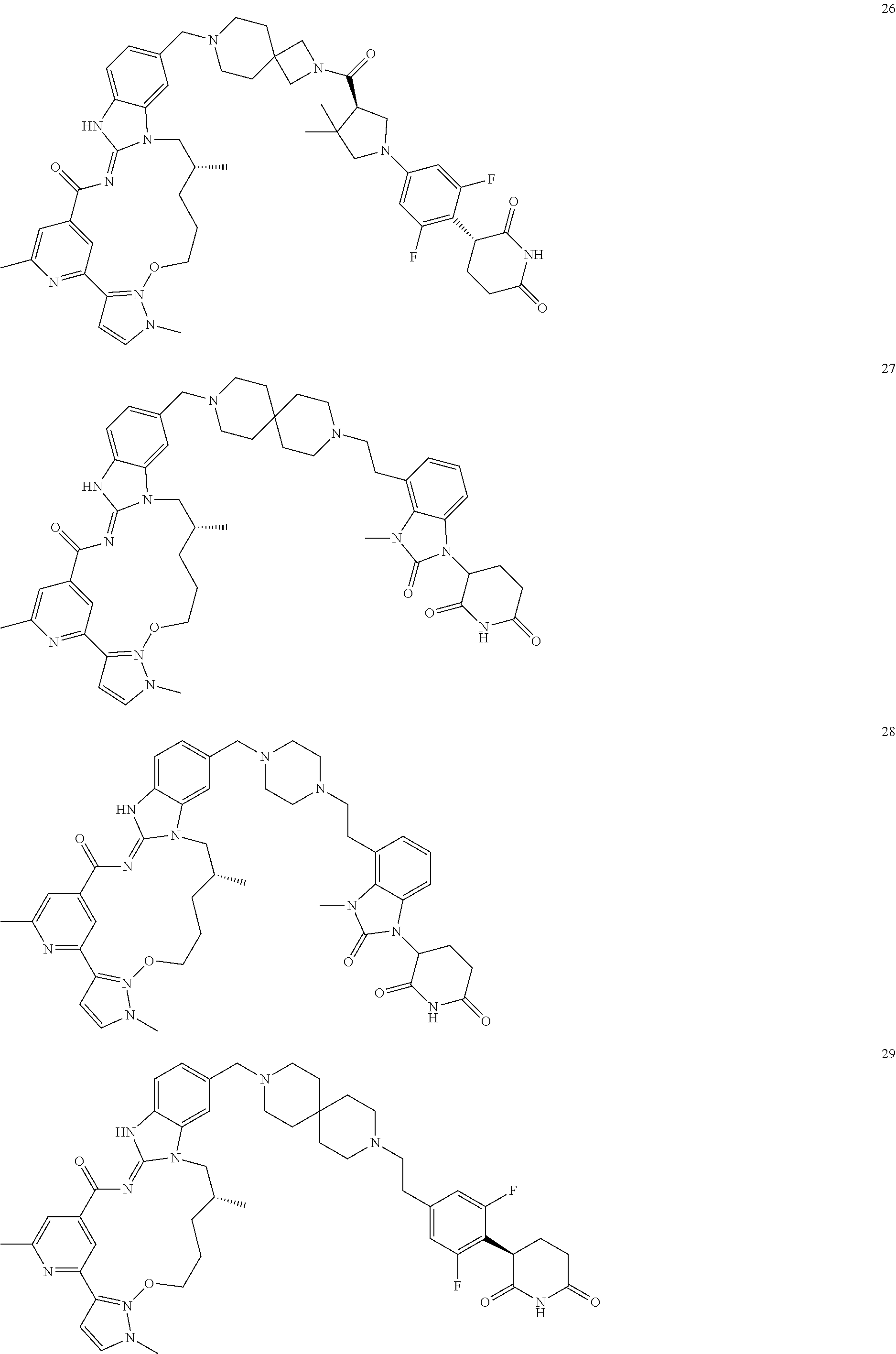

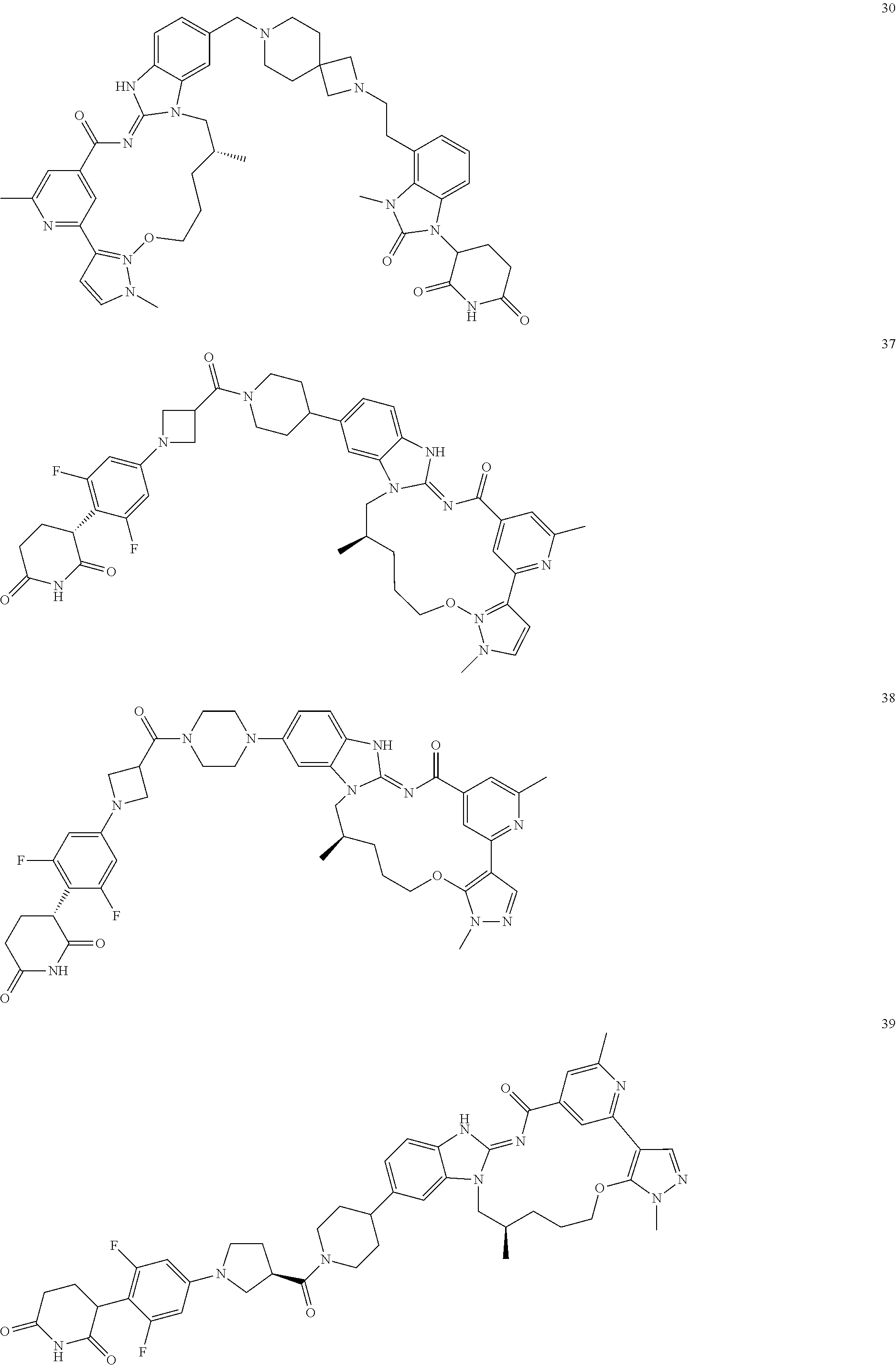

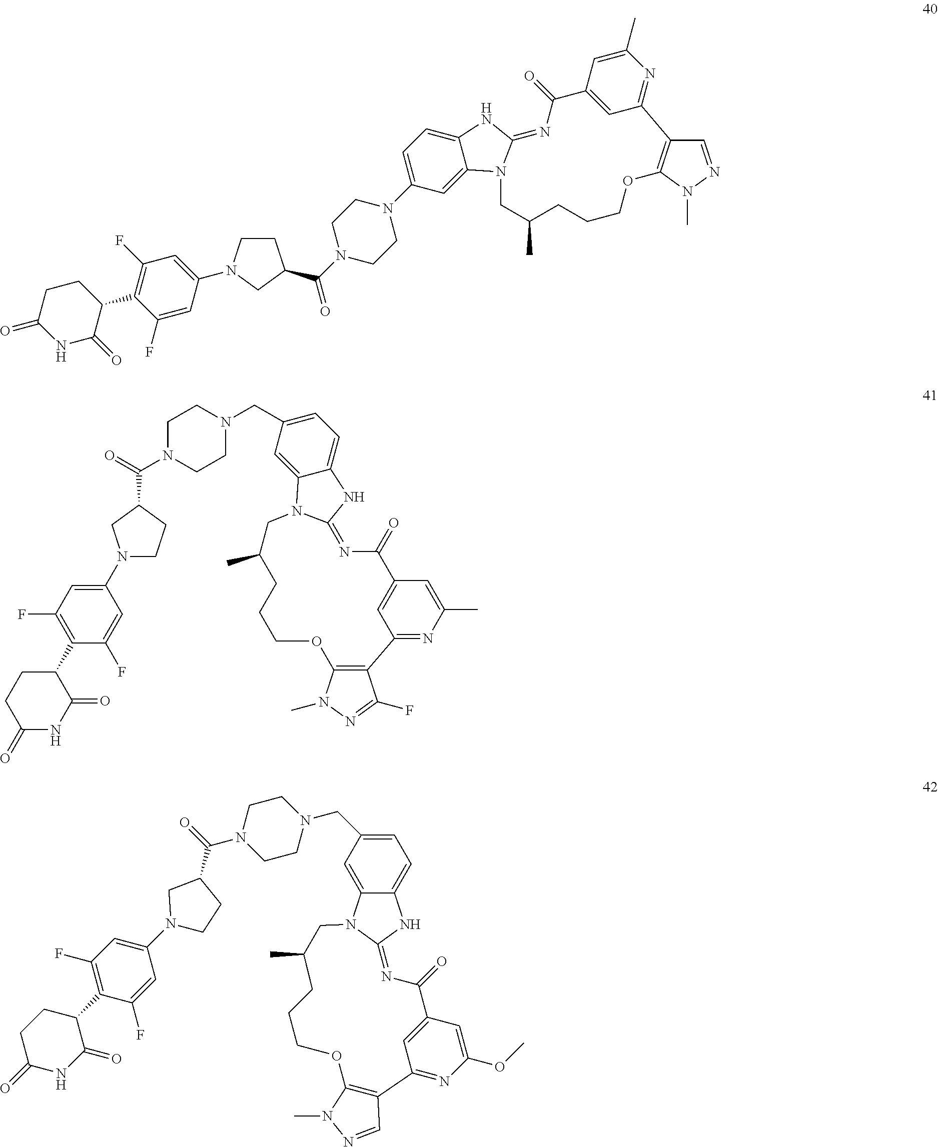 [SMILES: Cc1cc2cc(n1)-c1cnn(C)c1OCCC[C@@H](C)CN1/C(=N/C2=O)Nc2ccc(N3CCN(C(=O)[C@@H]4CCN(c5cc(F)c([C@H]6CCC(=O)NC6=O)c(F)c5)C4)CC3)cc21 ; Cc1cc2cc(n1)-c1c(F)nn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21 ; COc1cc2cc(n1)-c1cnn(C)c1OCCC[C@@H](C)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21]

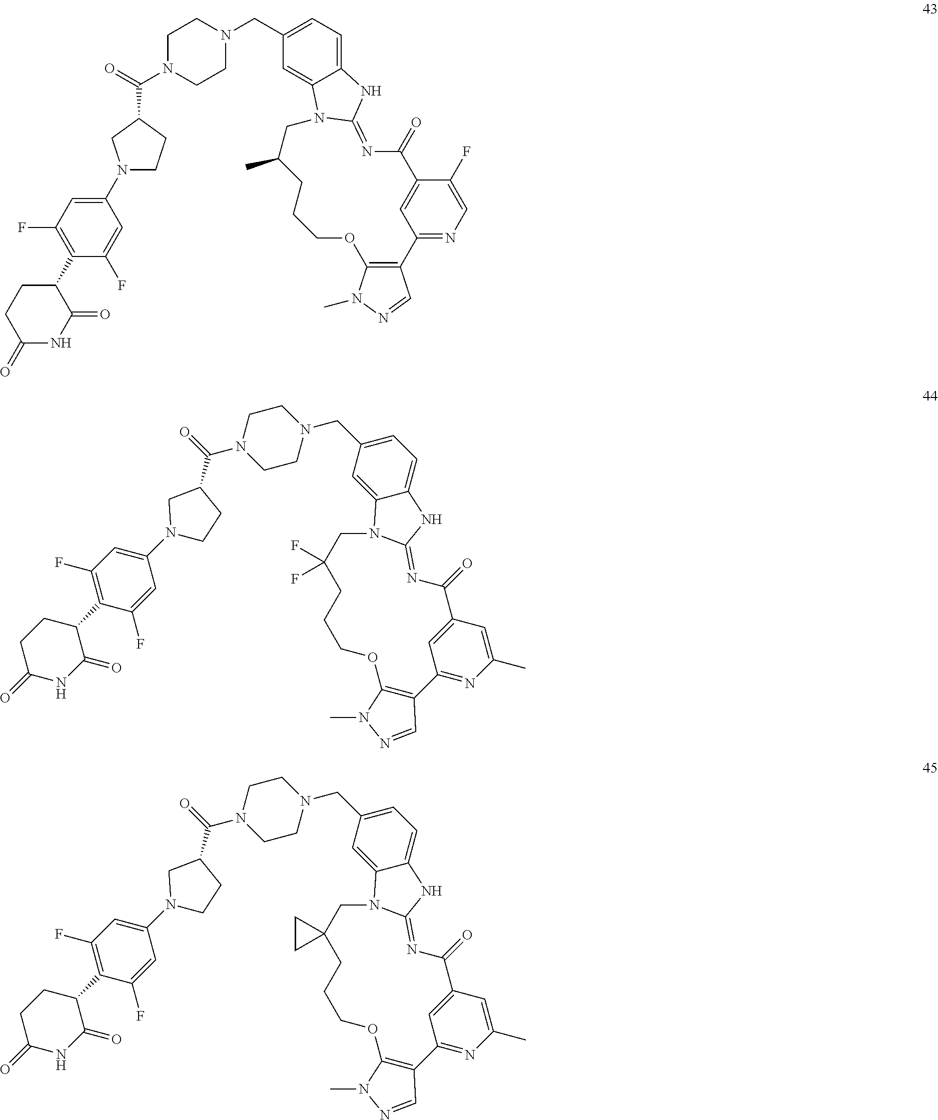 [SMILES: C[C@@H]1CCCOc2c(cnn2C)-c2cc(c(F)cn2)C(=O)/N=C2\Nc3ccc(CN4CCN(C(=O)[C@@H]5CCN(c6cc(F)c([C@H]7CCC(=O)NC7=O)c(F)c6)C5)CC4)cc3N2C1 ; Cc1cc2cc(n1)-c1cnn(C)c1OCCCC(F)(F)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21 ; Cc1cc2cc(n1)-c1cnn(C)c1OCCCC1(CC1)CN1/C(=N/C2=O)Nc2ccc(CN3CCN(C(=O)[C@@H]4CCN(c5cc(F)c([C@H]6CCC(=O)NC6=O)c(F)c5)C4)CC3)cc21]

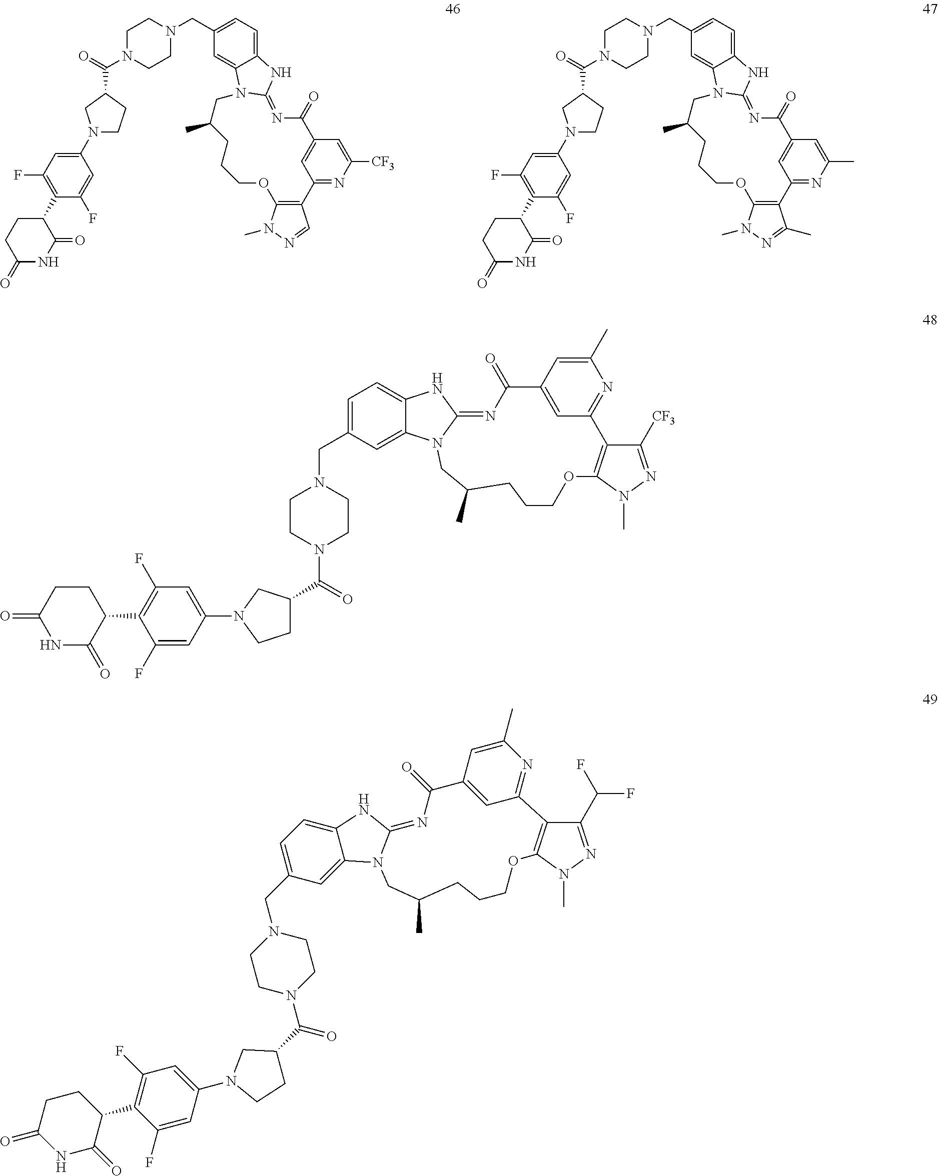

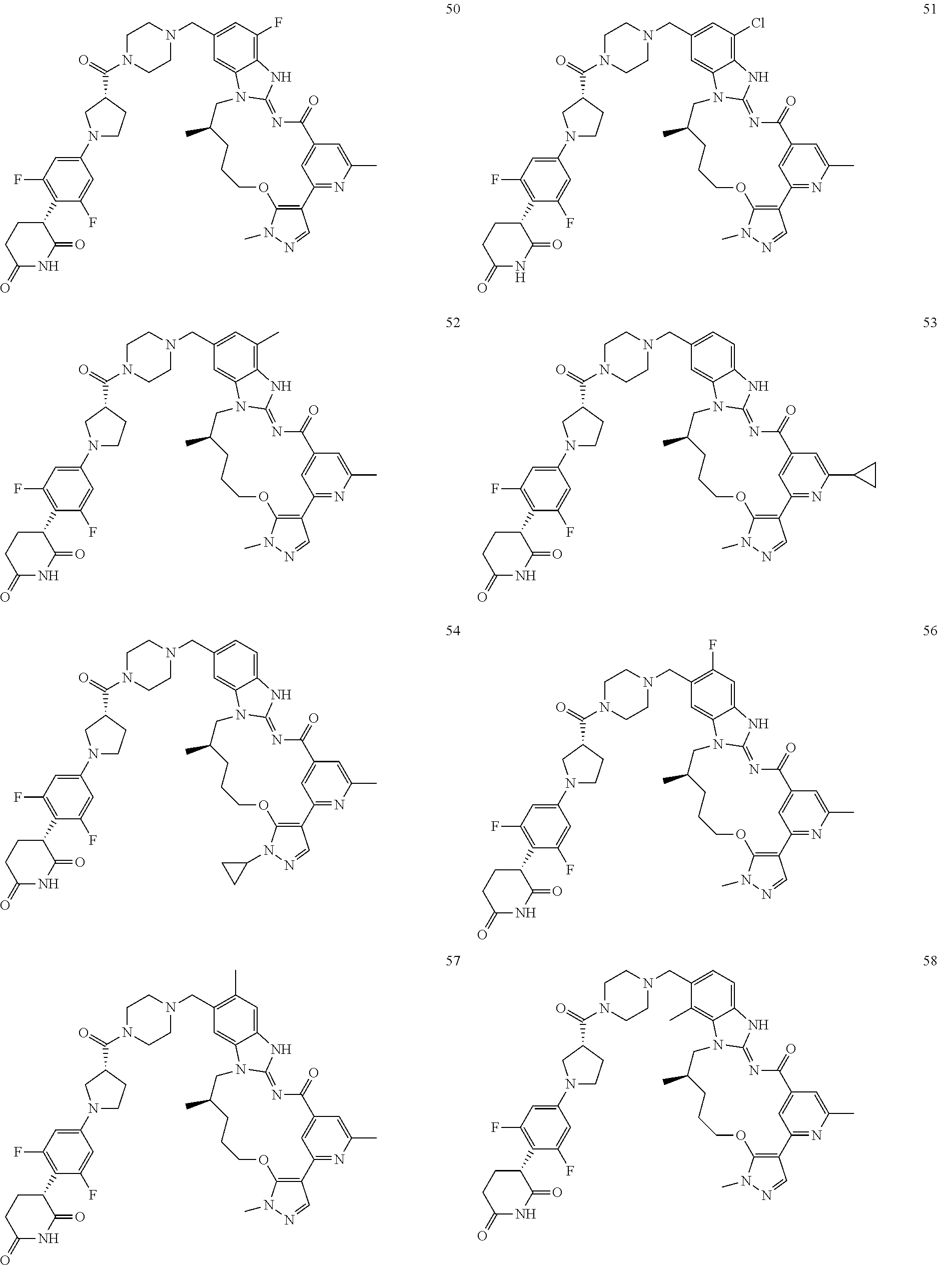

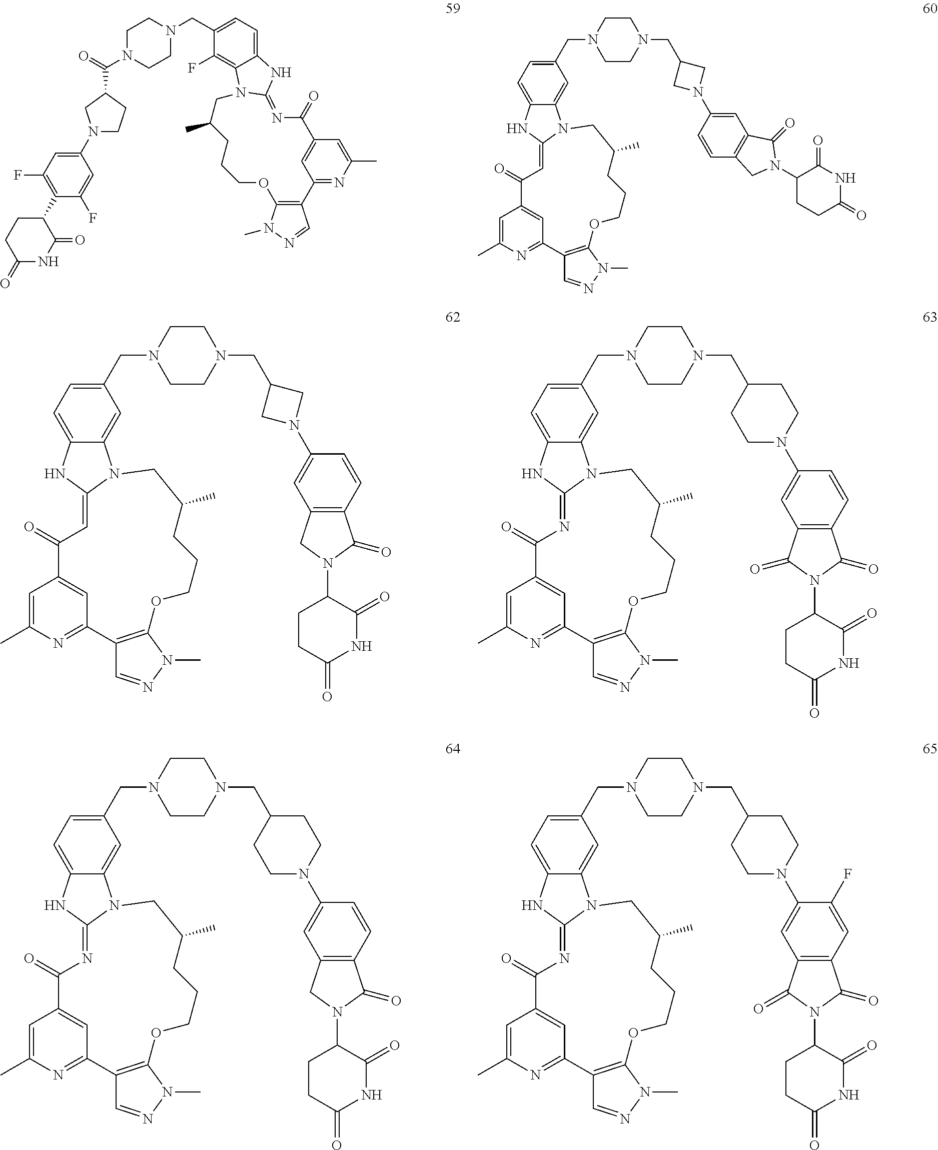

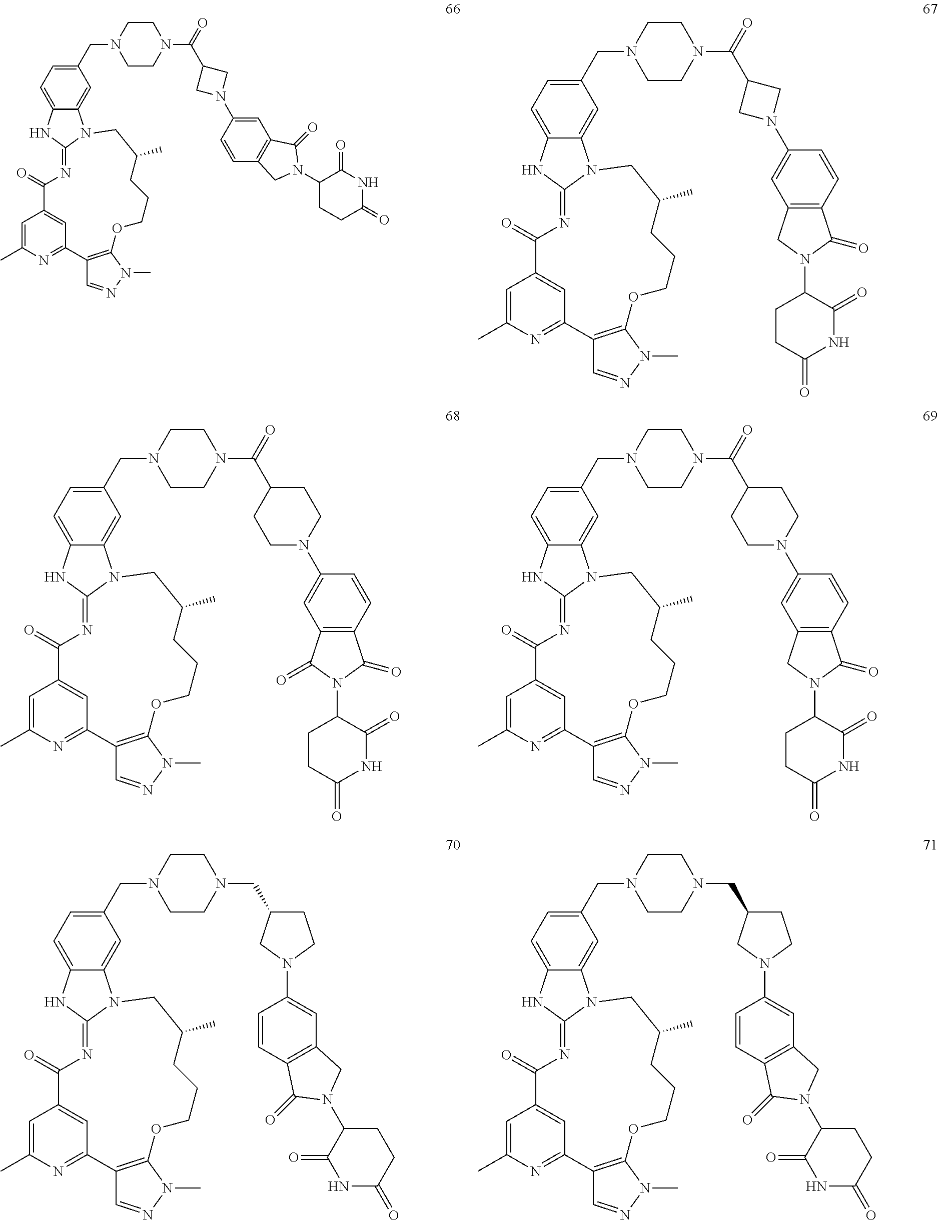

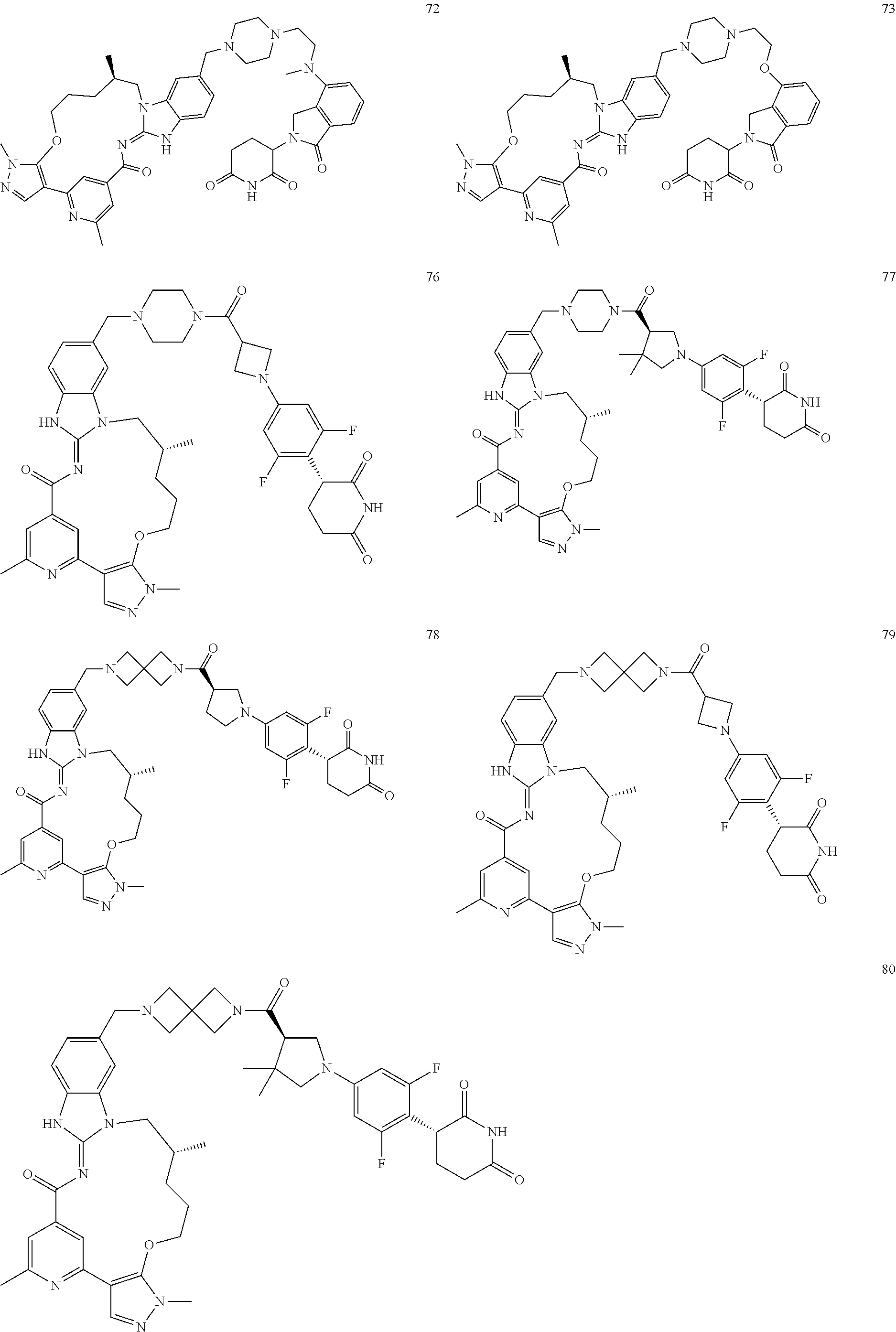

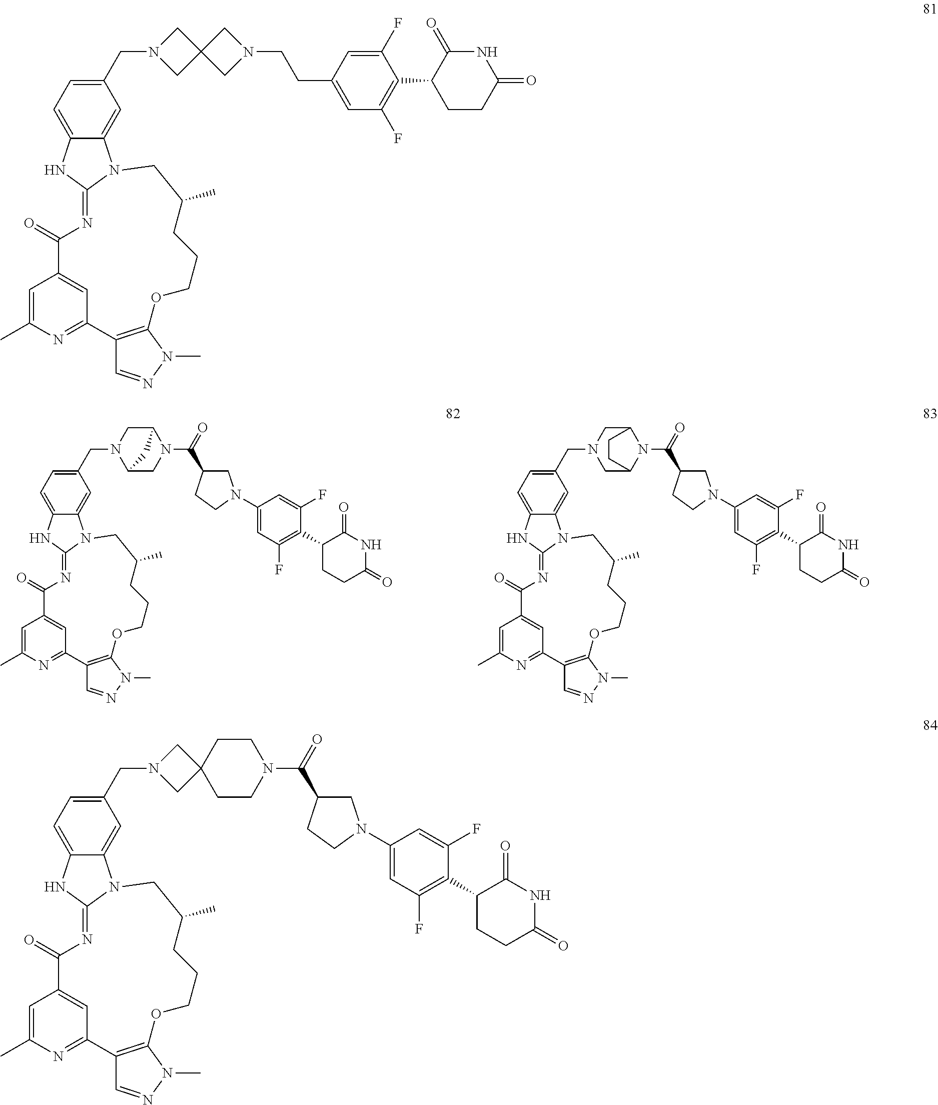

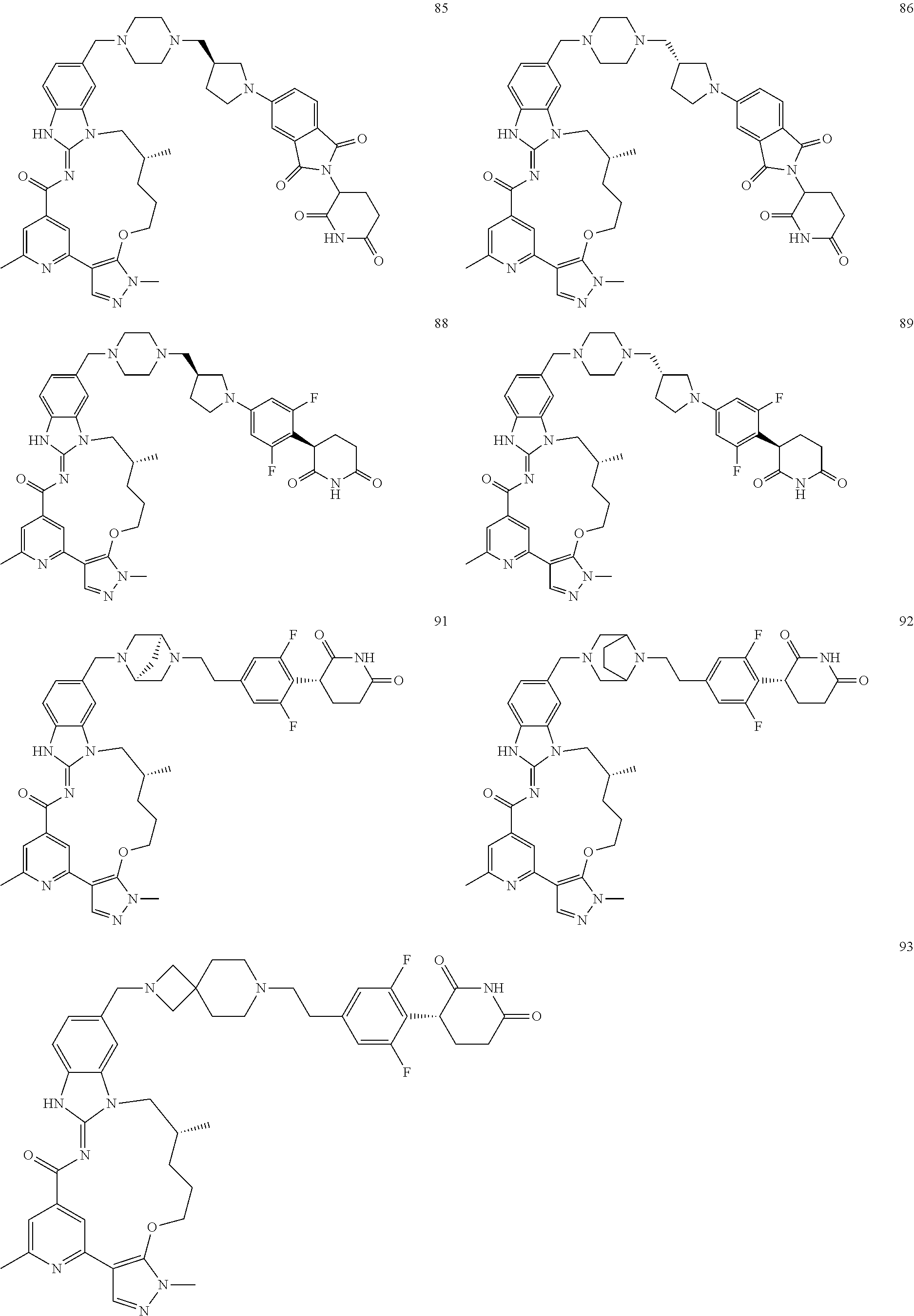

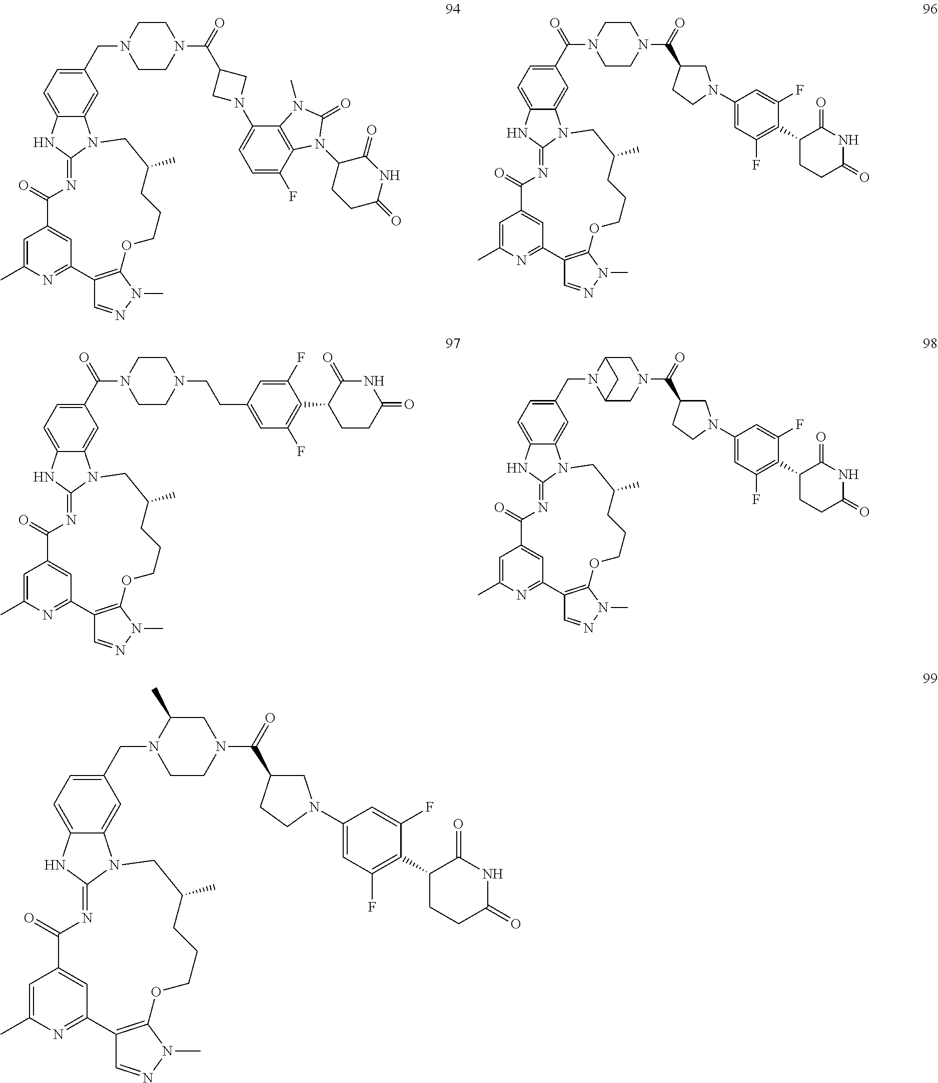

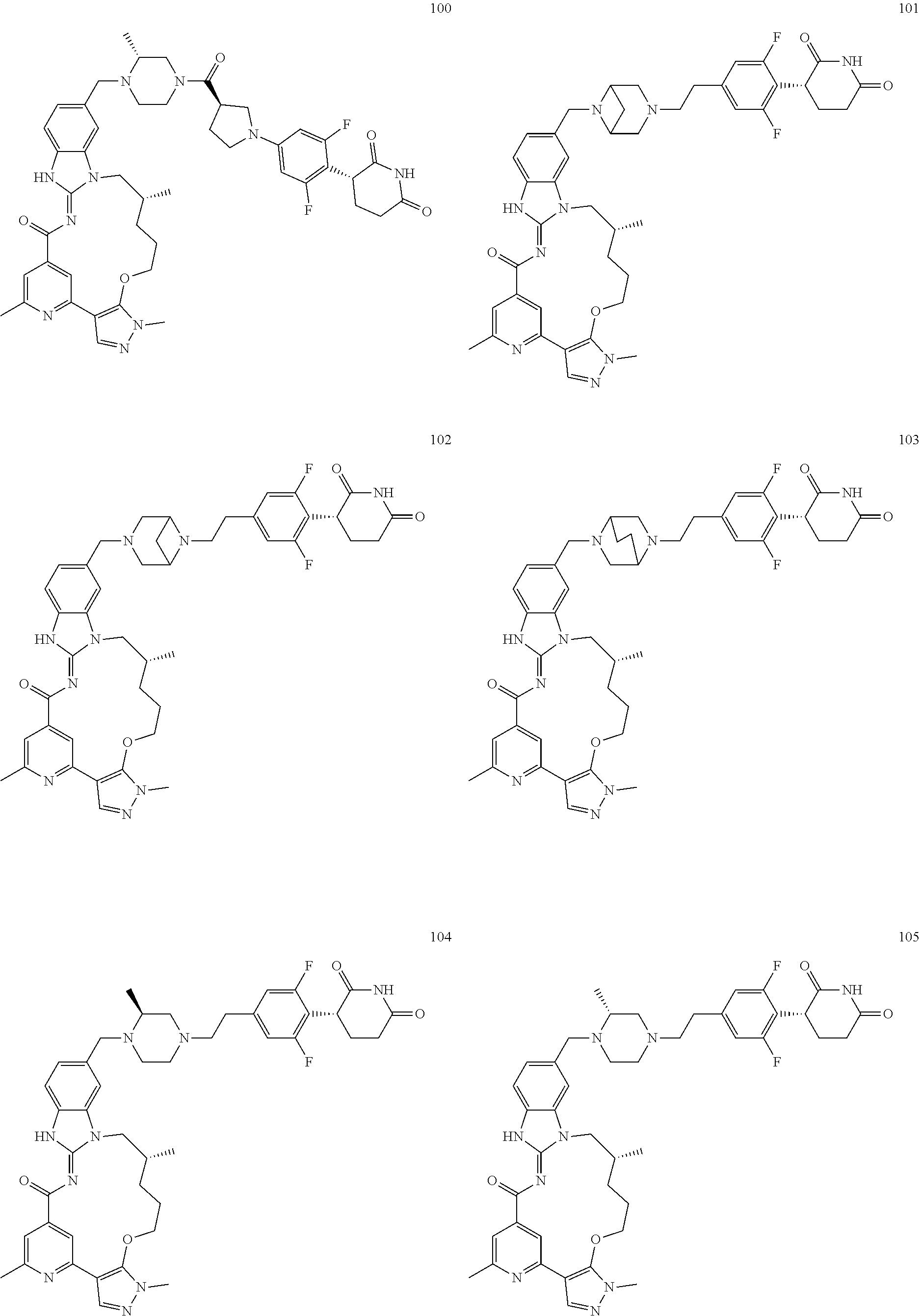

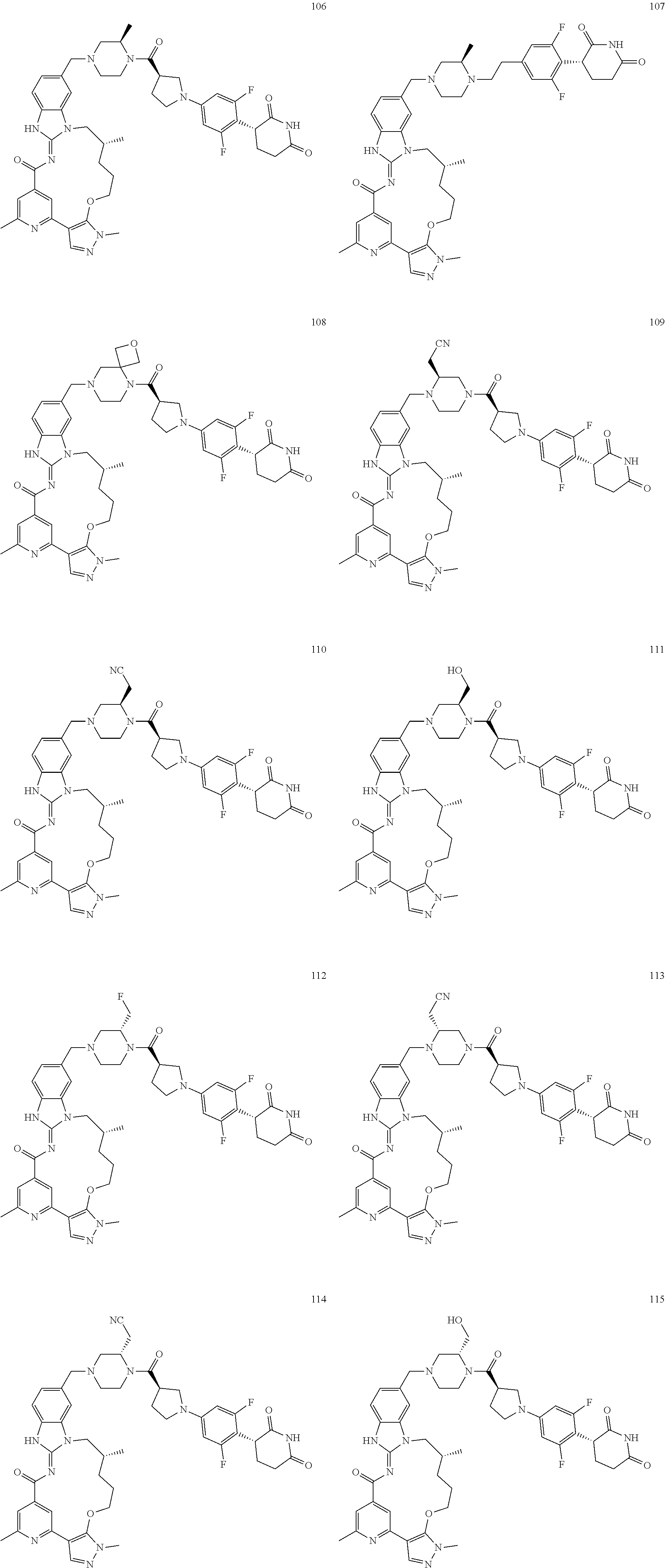

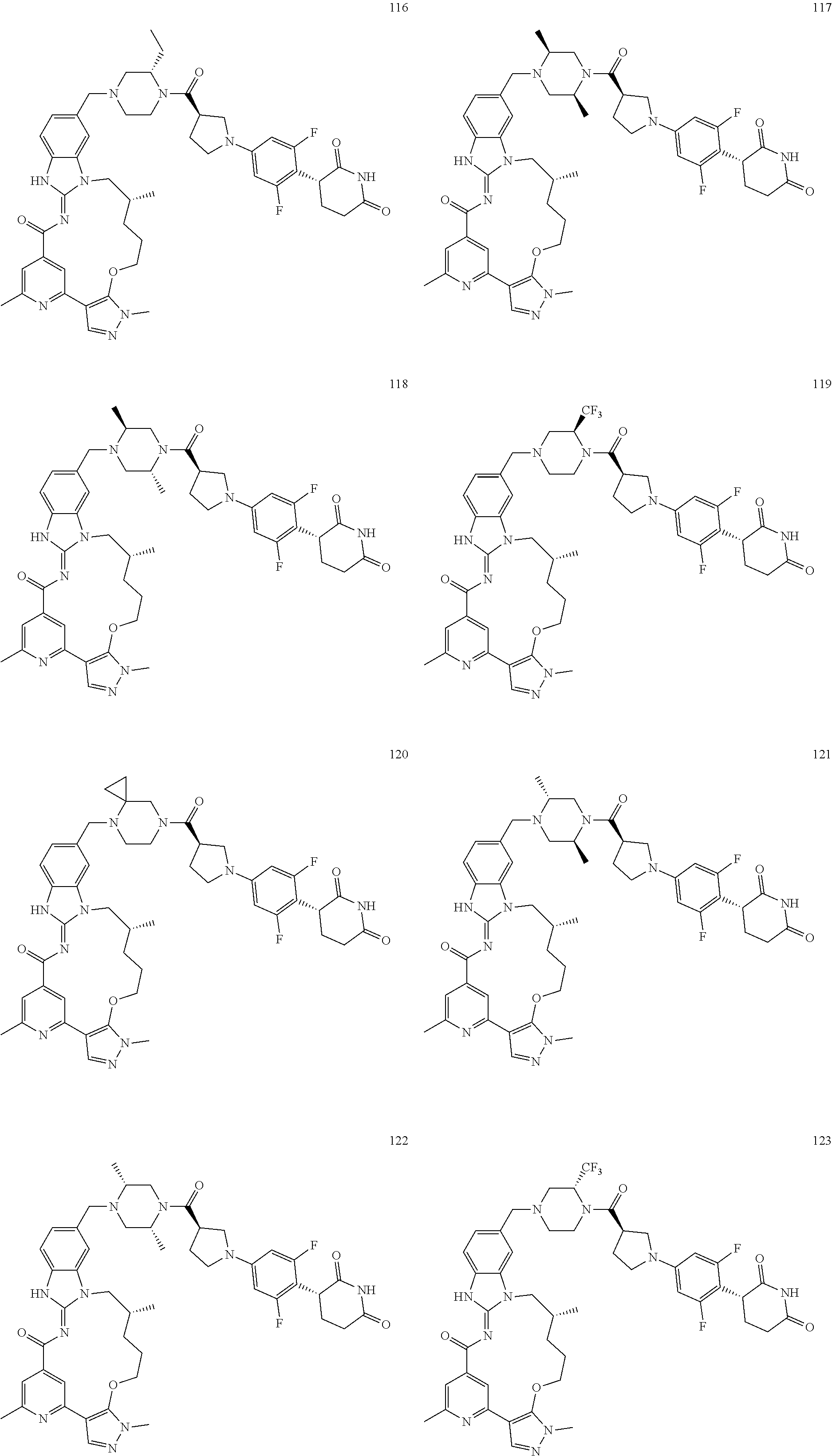

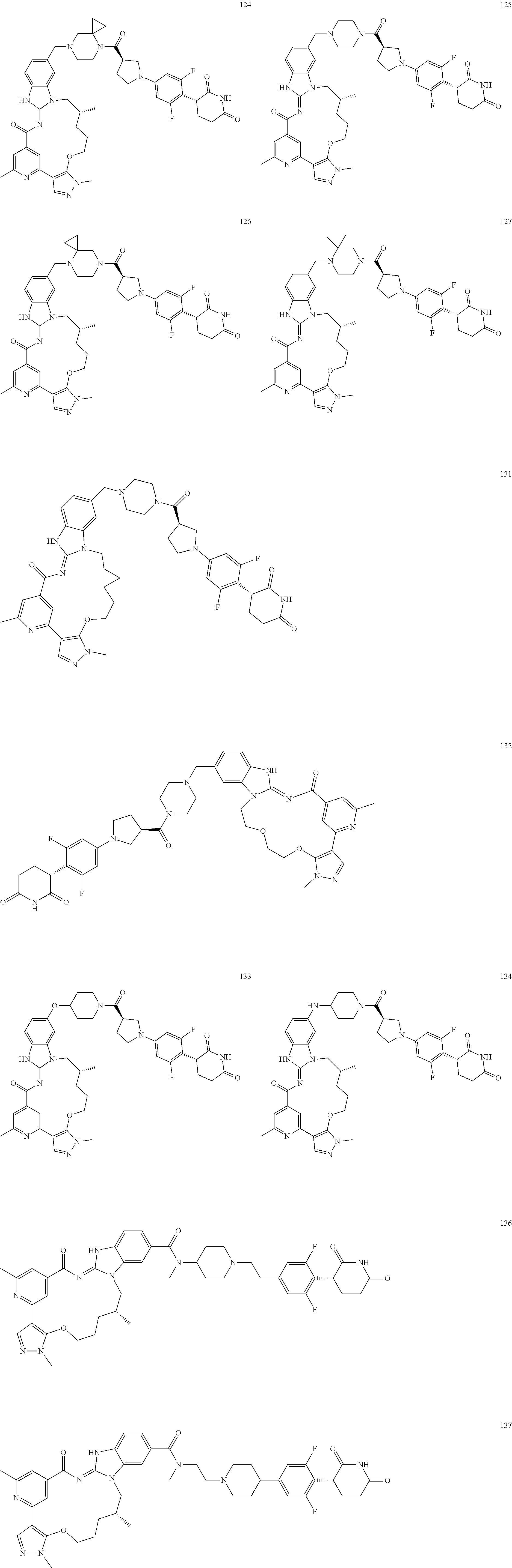

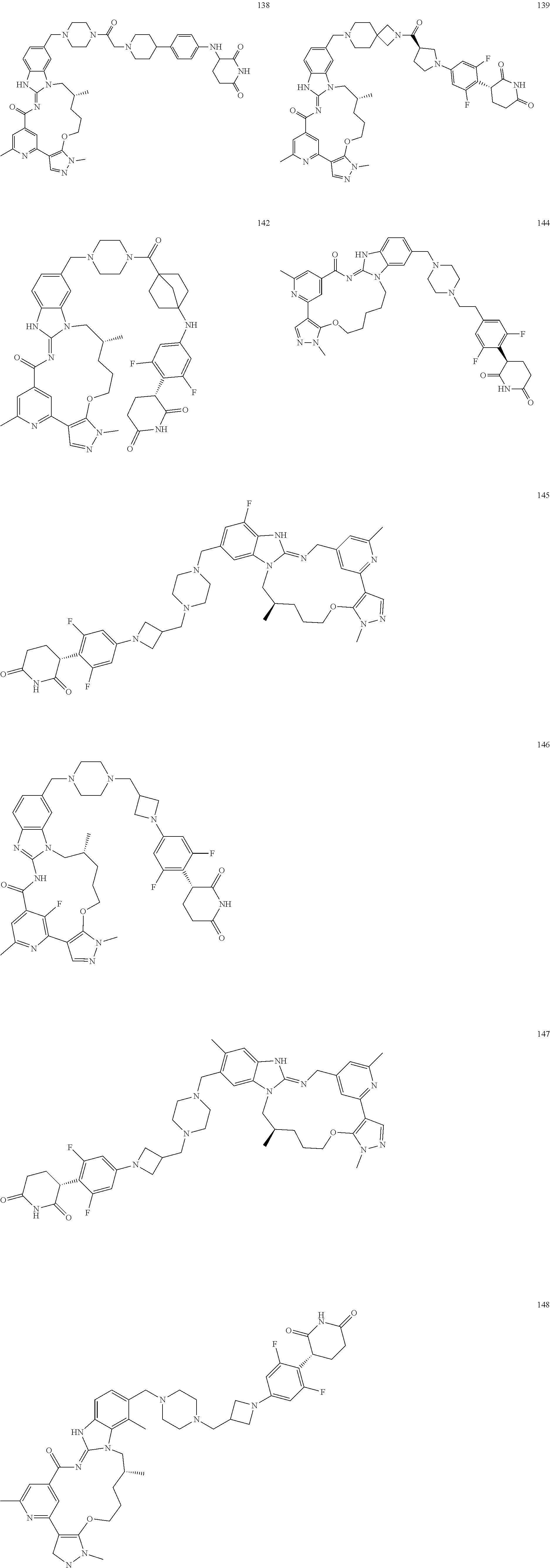

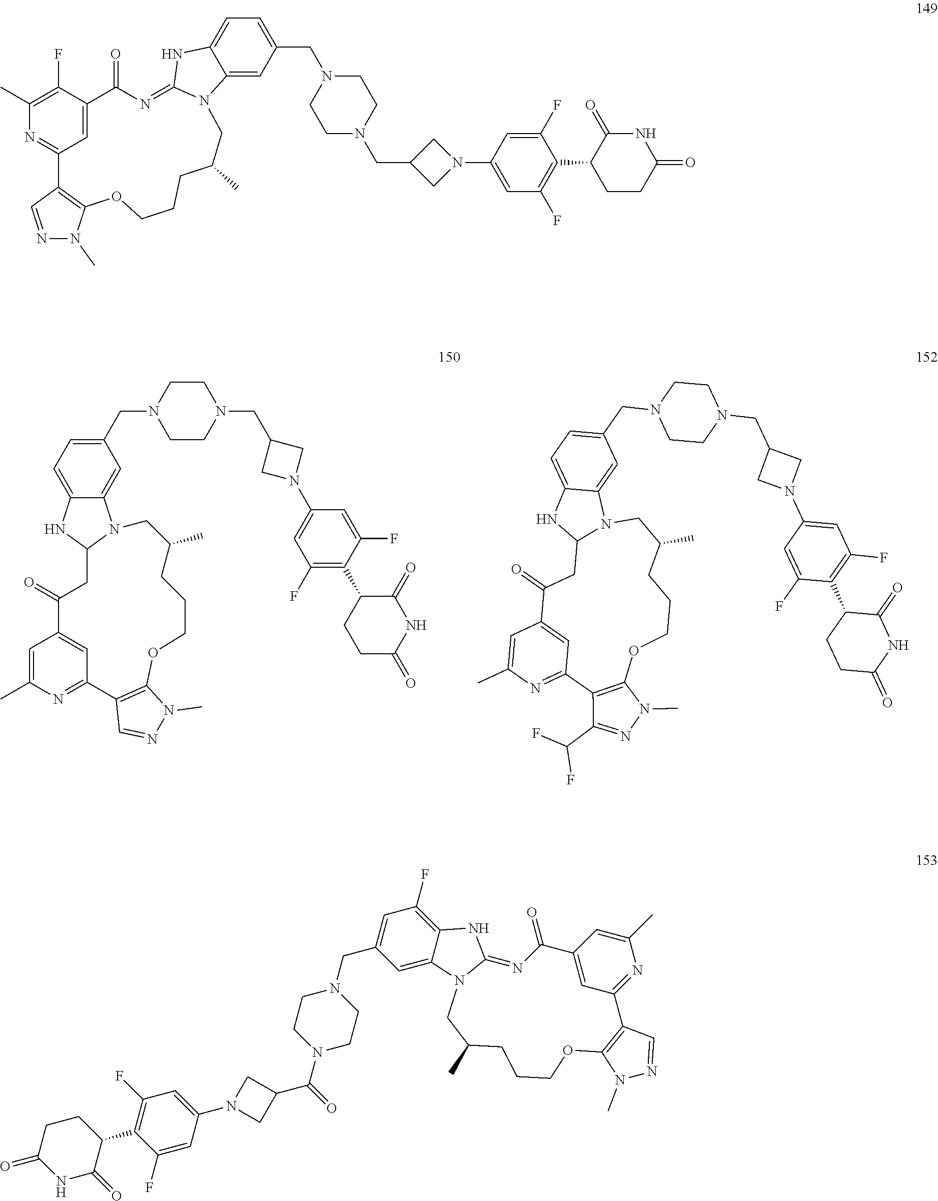 [SMILES: Cc1nc2cc(c1F)C(=O)/N=C1\Nc3ccc(CN4CCN(CC5CN(c6cc(F)c([C@H]7CCC(=O)NC7=O)c(F)c6)C5)CC4)cc3N1C[C@H](C)CCCOc1c-2cnn1C ; Cc1cc2cc(n1)-c1cnn(C)c1OCCC[C@@H](C)CN1c3cc(CN4CCN(CC5CN(c6cc(F)c([C@H]7CCC(=O)NC7=O)c(F)c6)C5)CC4)ccc3NC1CC2=O ; Cc1cc2cc(n1)-c1c(C(F)F)nn(C)c1OCCC[C@@H](C)CN1c3cc(CN4CCN(CC5CN(c6cc(F)c([C@H]7CCC(=O)NC7=O)c(F)c6)C5)CC4)ccc3NC1CC2=O ; Cc1cc2cc(n1)-c1cnn(C)c1OCCC[C@@H](C)CN1/C(=N/C2=O)Nc2c(F)cc(CN3CCN(C(=O)C4CN(c5cc(F)c([C@H]6CCC(=O)NC6=O)c(F)c5)C4)CC3)cc21]

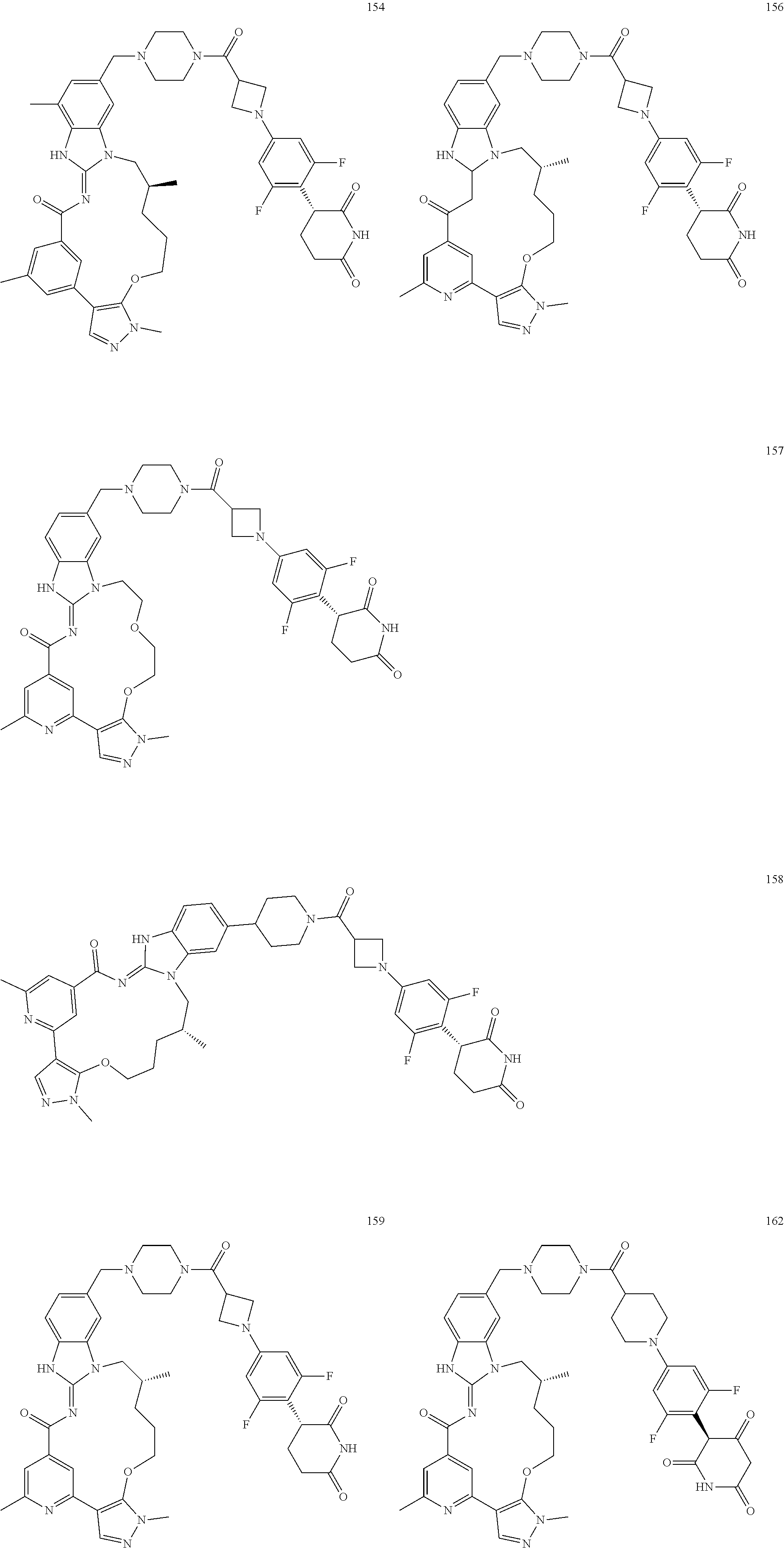

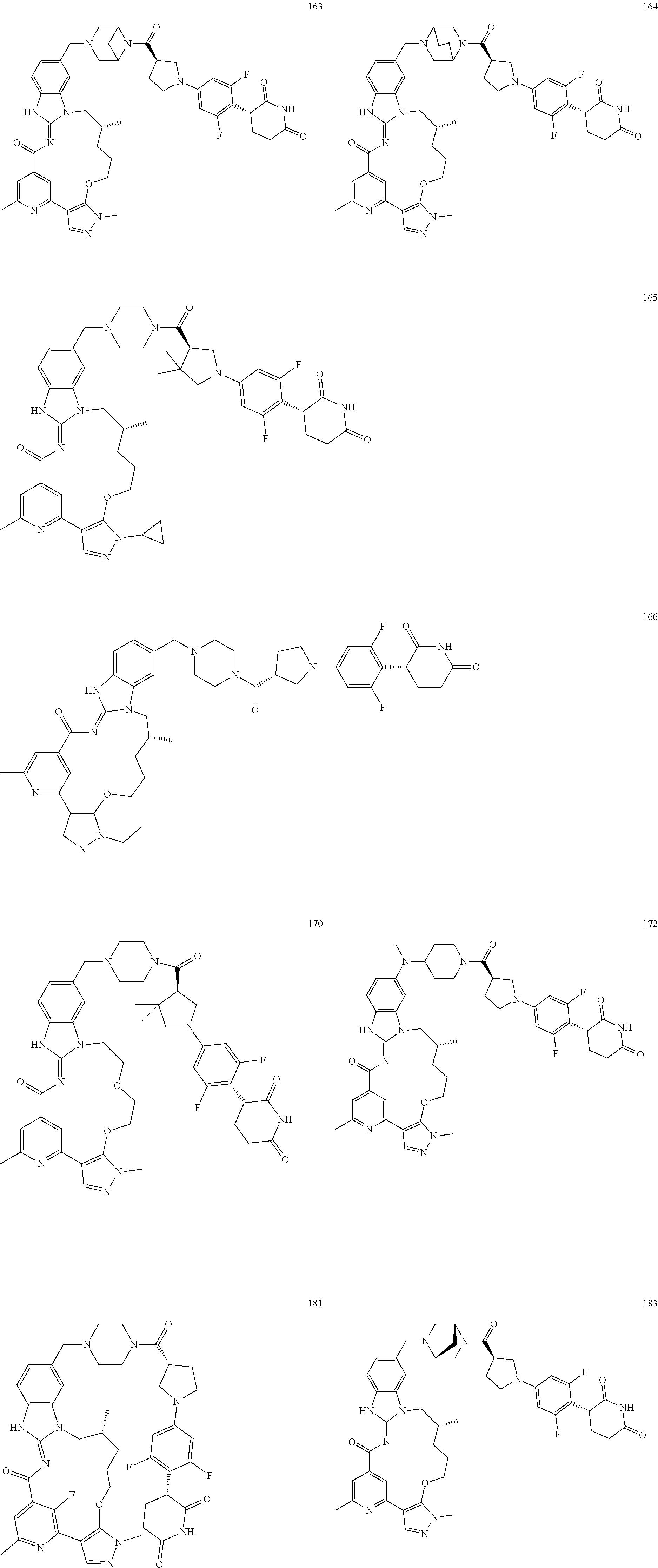

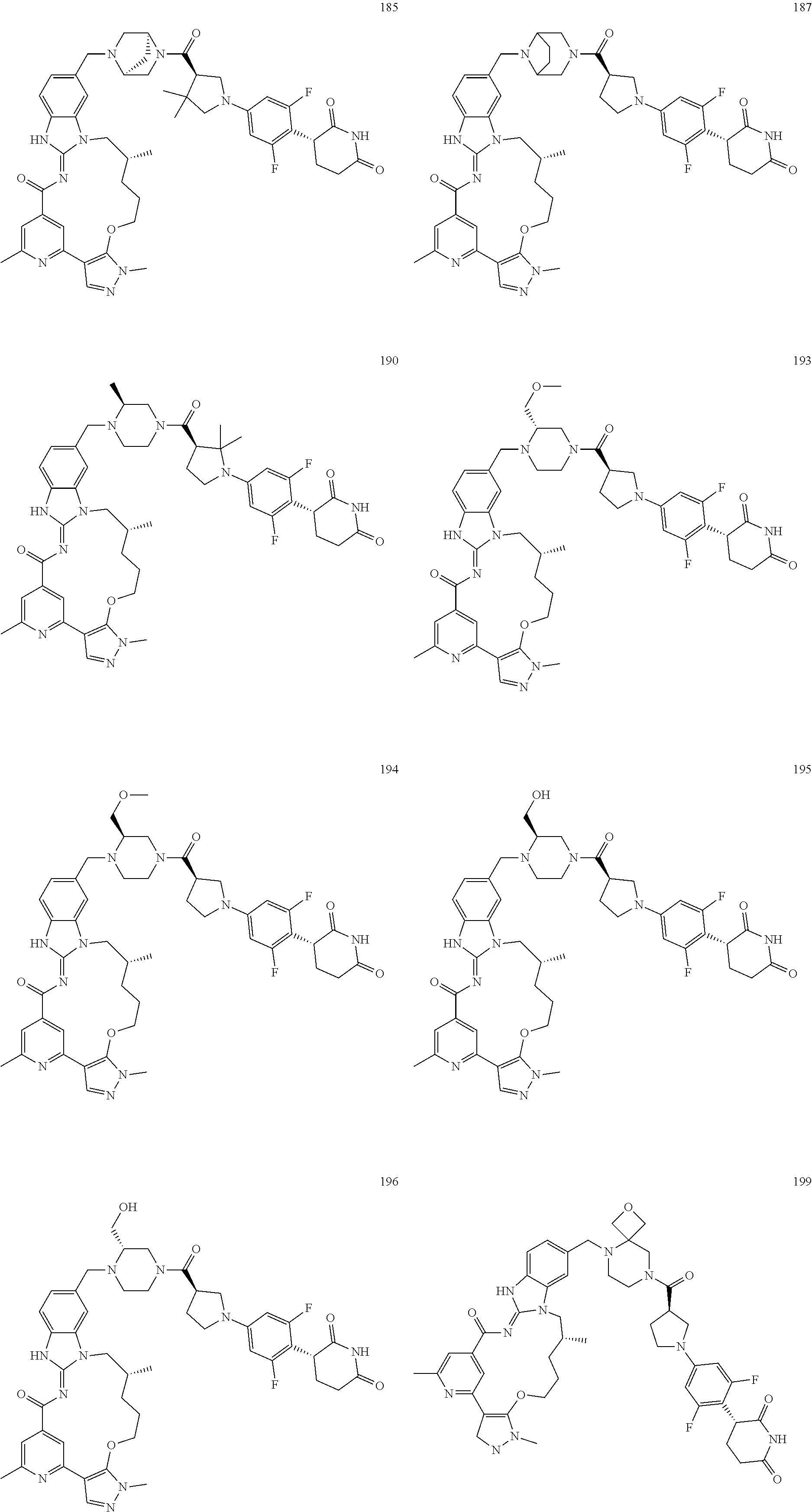

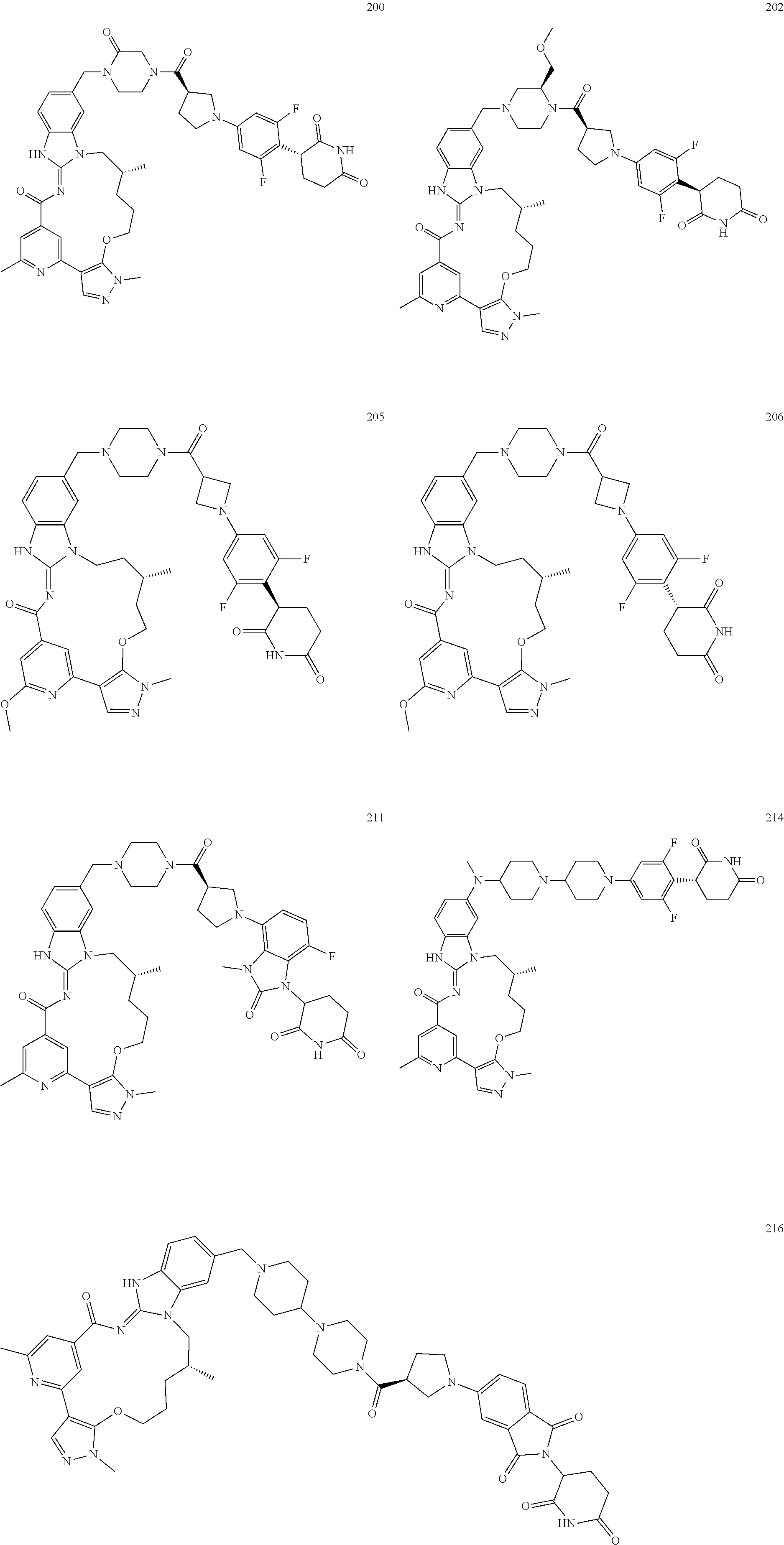

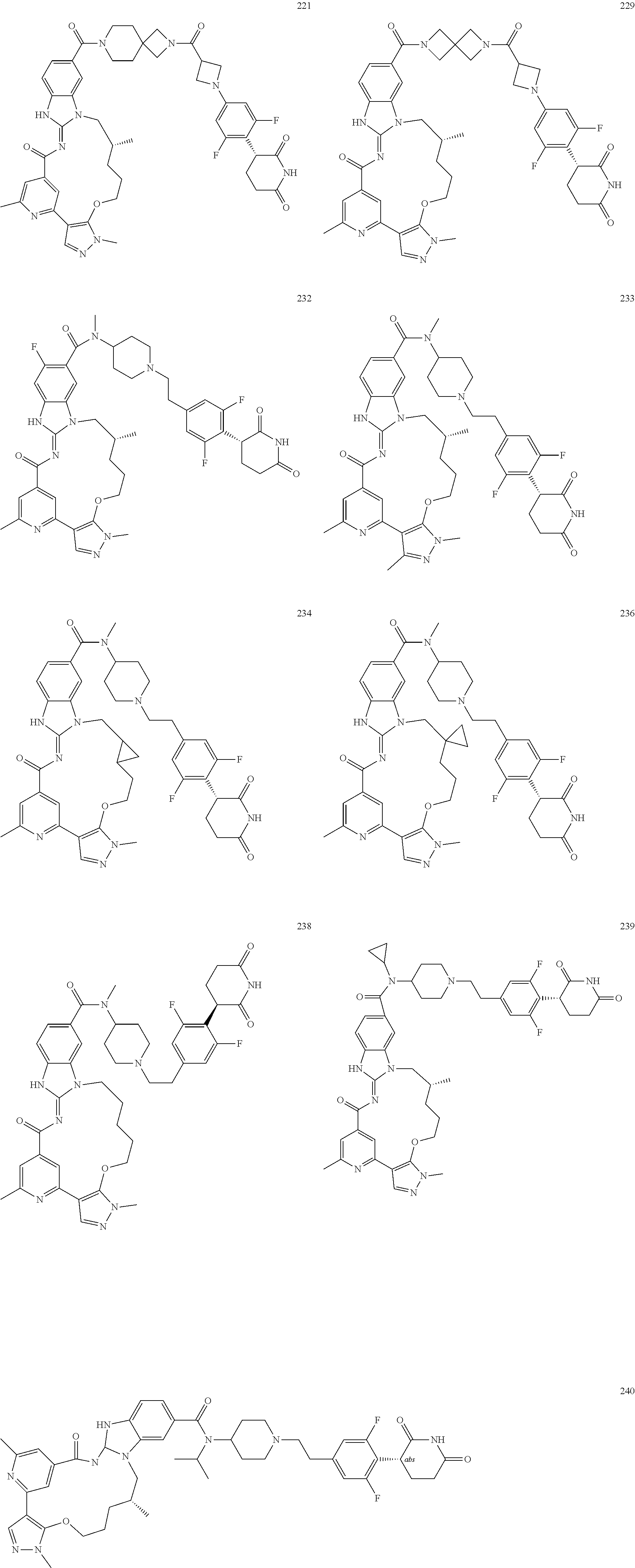

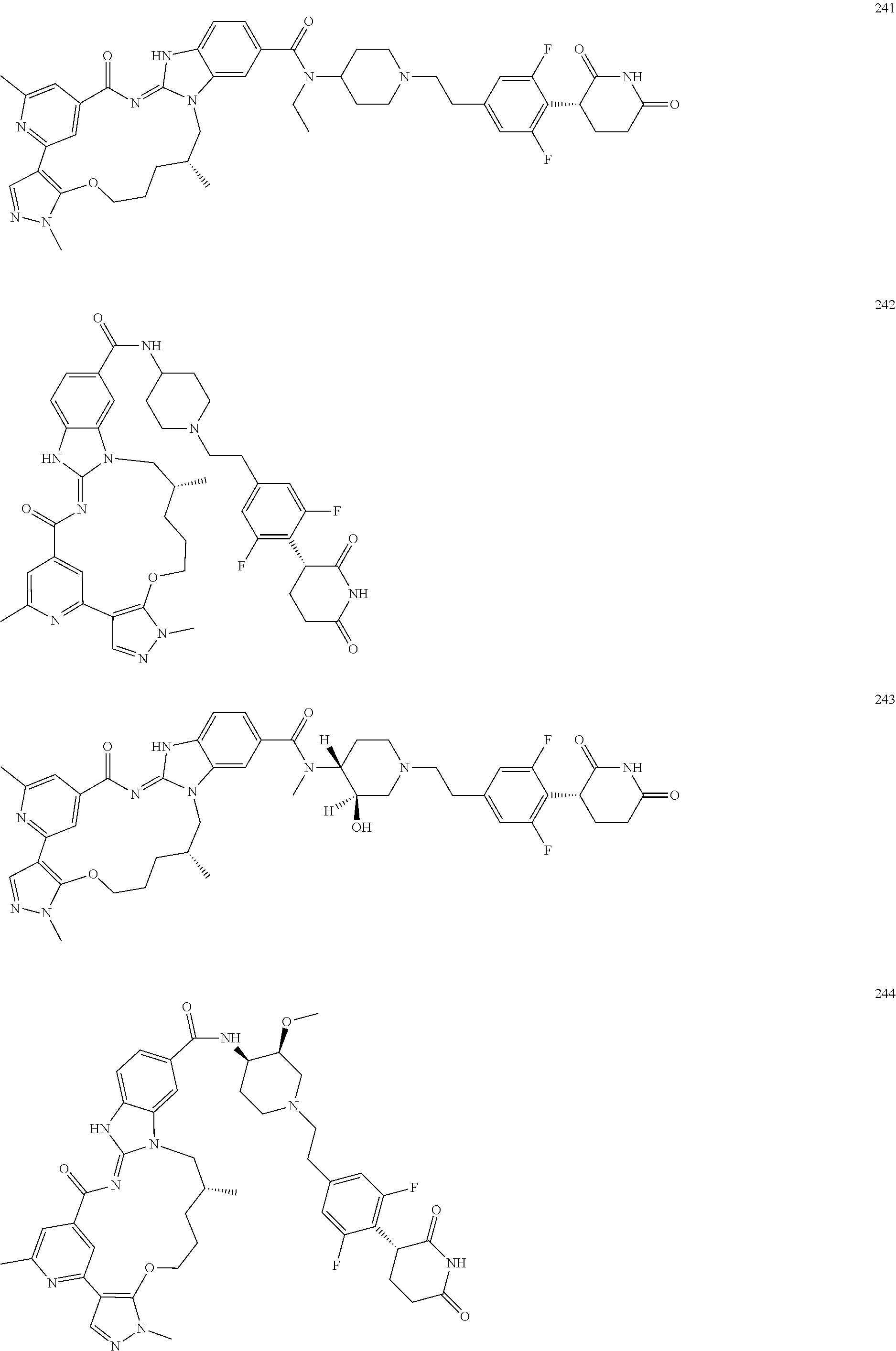 [SMILES: CCN(C(=O)c1ccc2c(c1)N1C[C@H](C)CCCOc3c(cnn3C)-c3cc(cc(C)n3)C(=O)/N=C/1N2)C1CCN(CCc2cc(F)c([C@H]3CCC(=O)NC3=O)c(F)c2)CC1 ; Cc1cc2cc(n1)-c1cnn(C)c1OCCC[C@@H](C)CN1/C(=N/C2=O)Nc2ccc(C(=O)NC3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C[C@H]3O)cc21 ; CO[C@H]1CN(CCc2cc(F)c([C@H]3CCC(=O)NC3=O)c(F)c2)CC[C@H]1NC(=O)c1ccc2c(c1)N1C[C@H](C)CCCOc3c(cnn3C)-c3cc(cc(C)n3)C(=O)/N=C/1N2]

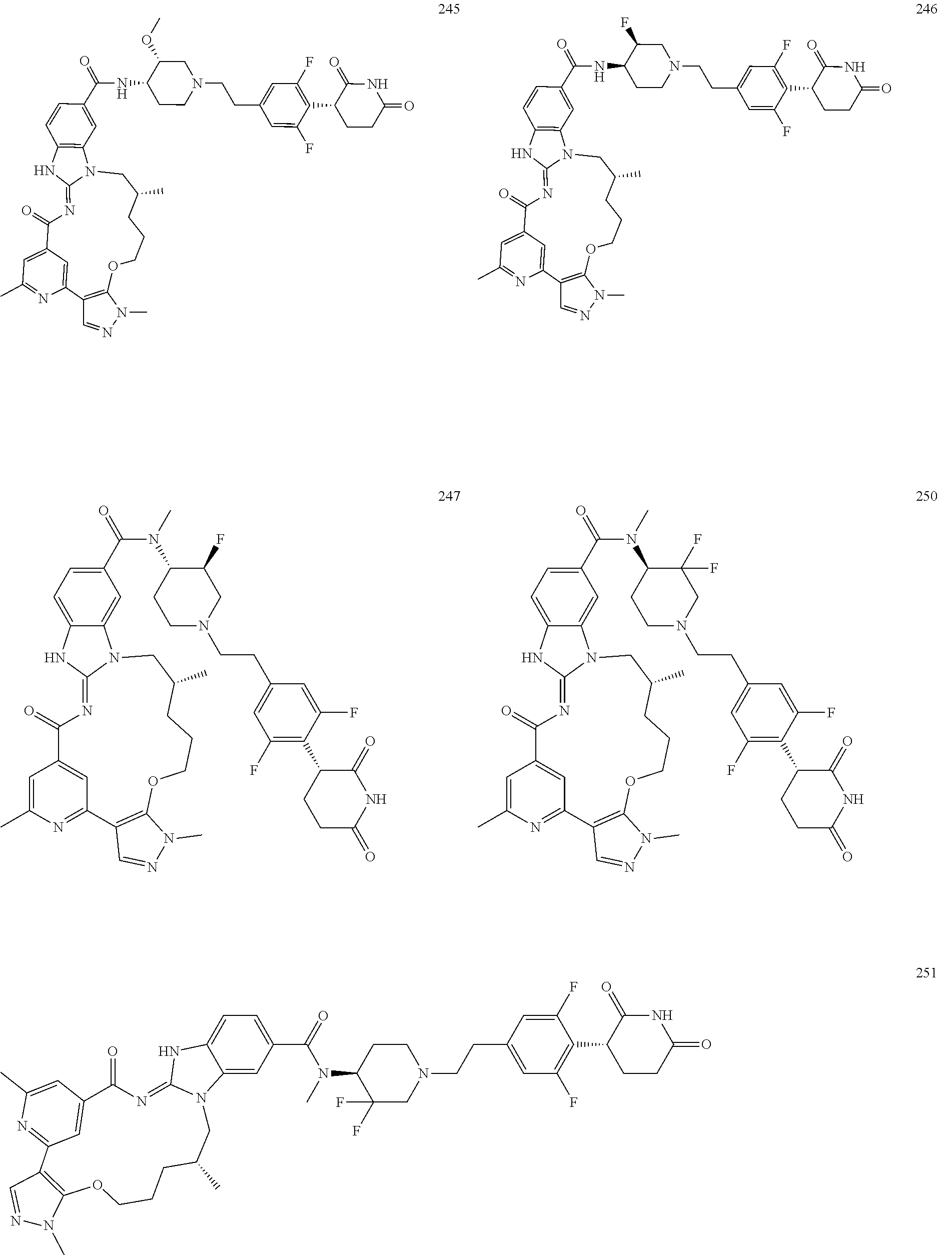

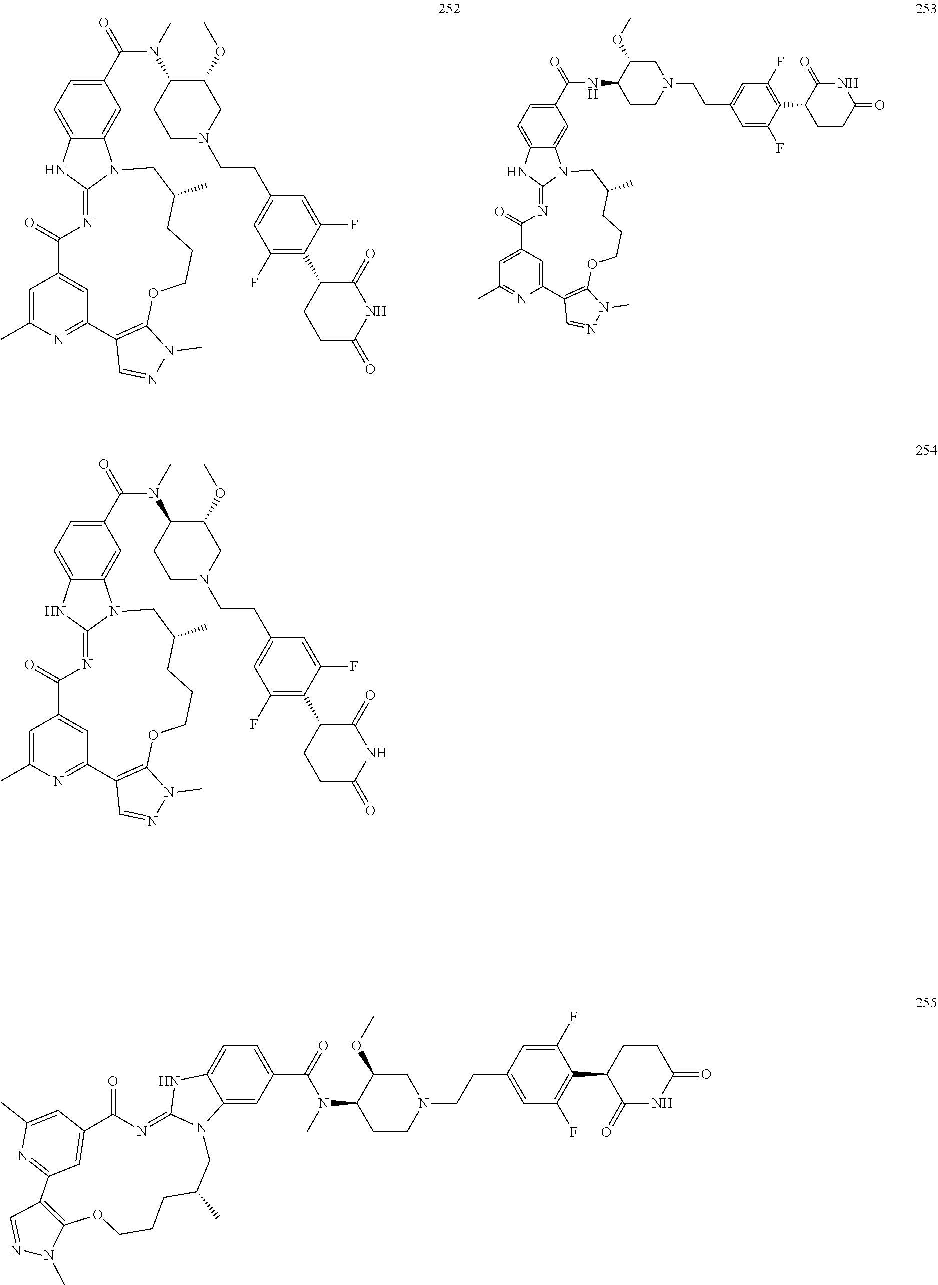

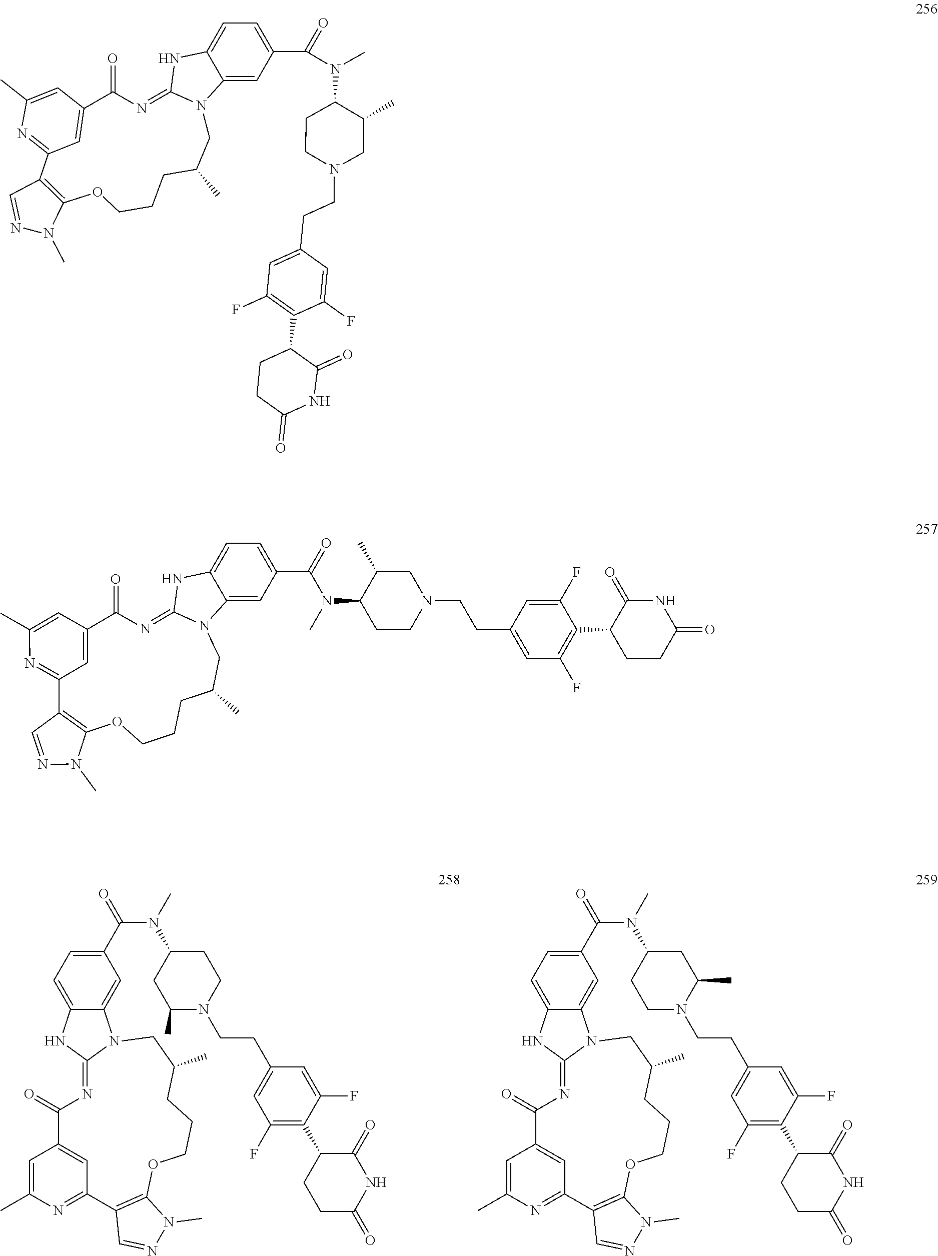

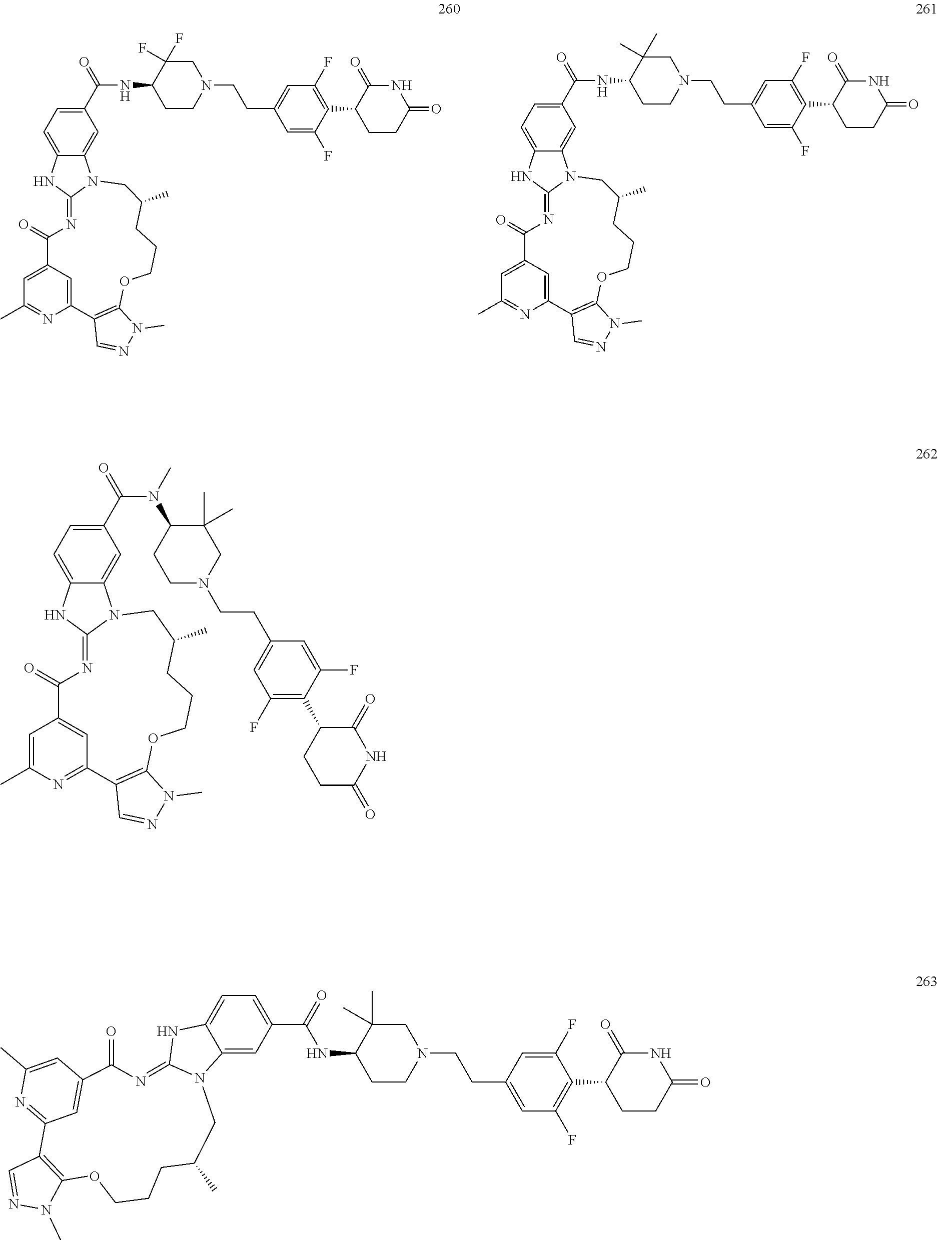

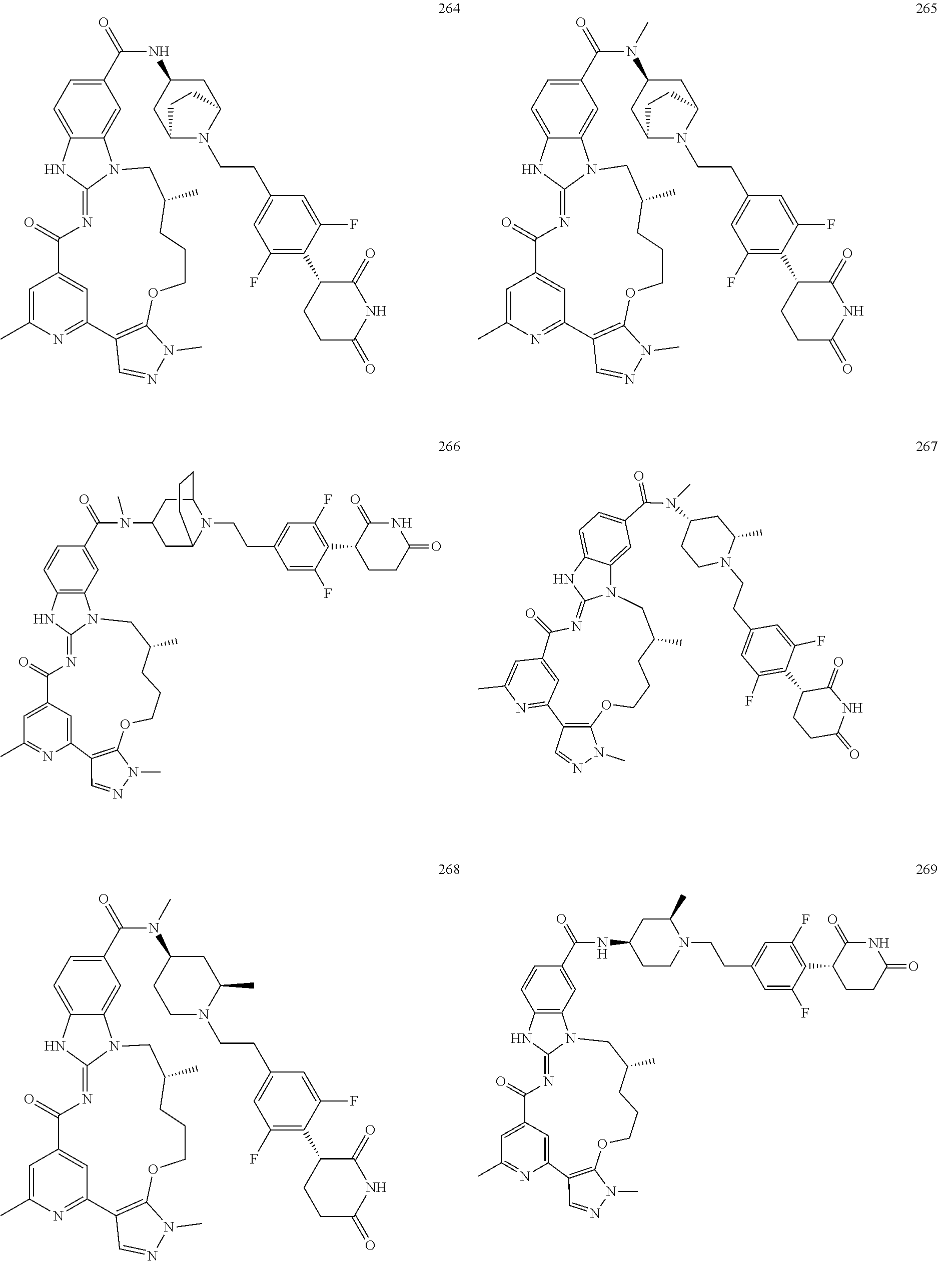

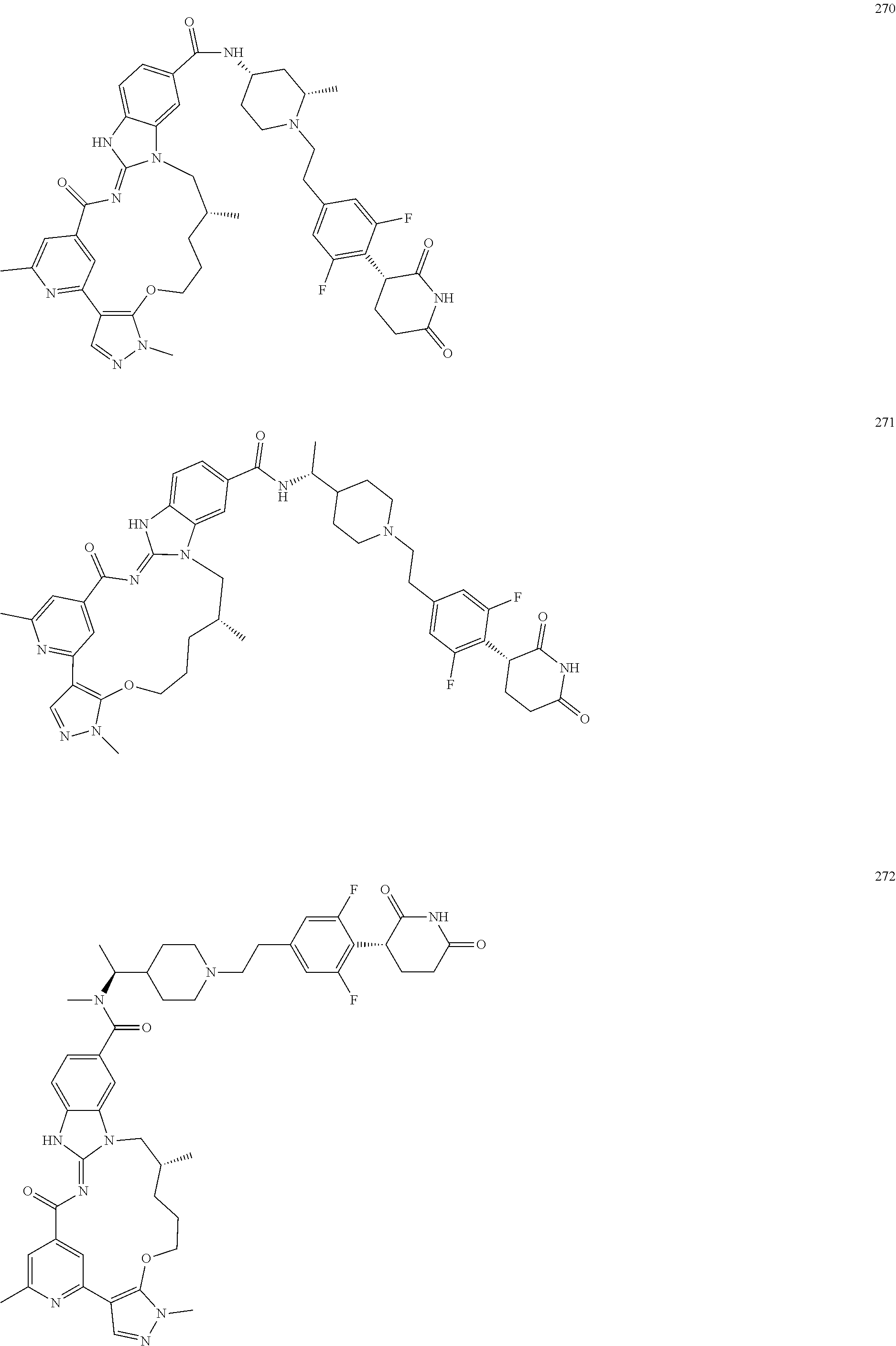 [SMILES: Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)[C@@H](C)C3)cc21 ; Cc1cc2cc(n1)-c1cnn(C)c1OCCC[C@@H](C)CN1/C(=N/C2=O)Nc2ccc(C(=O)N[C@H](C)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)[C@@H](C)C3CCN(CCc4cc(F)c([C@H]5CCC(=O)NC5=O)c(F)c4)CC3)cc21]

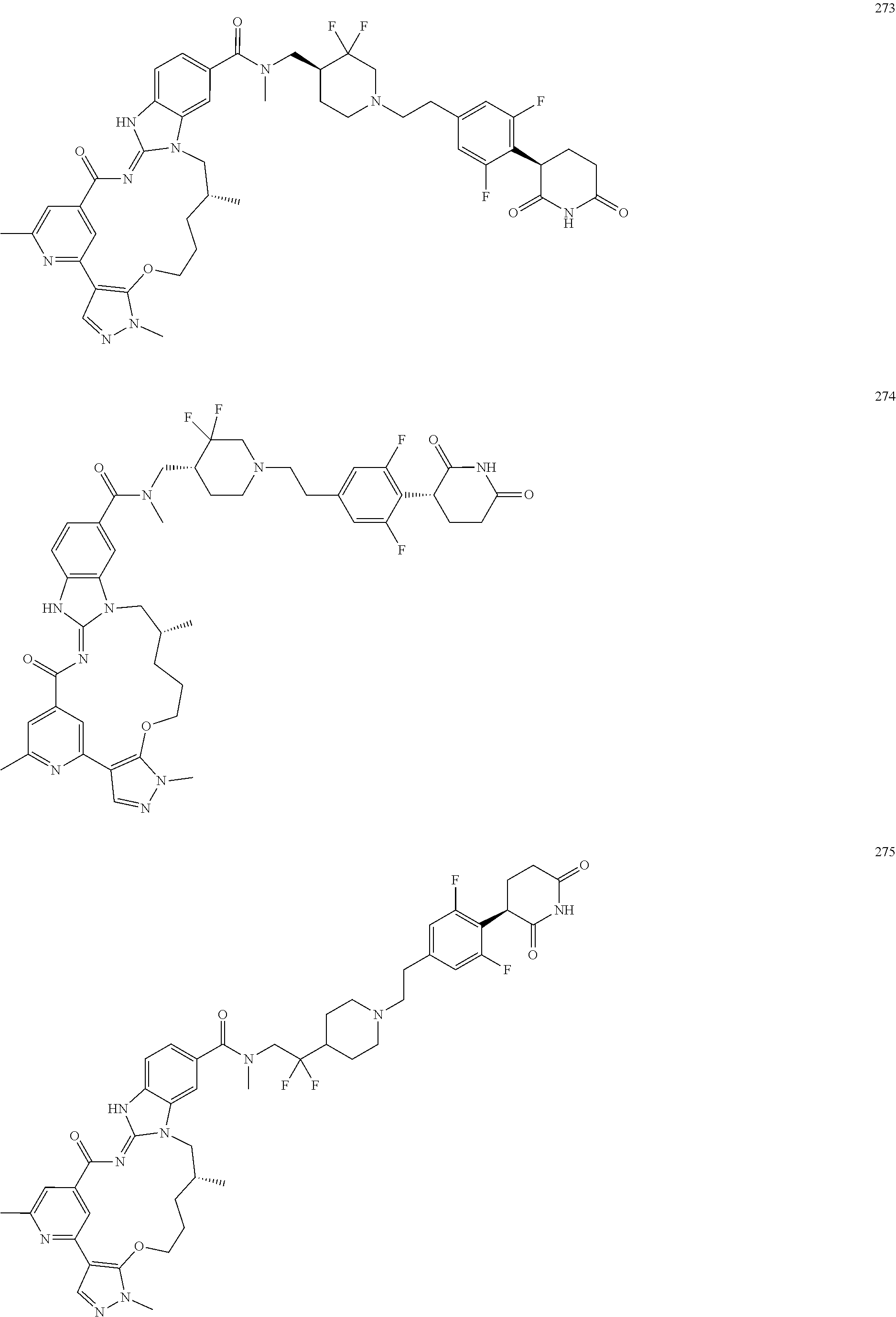 [SMILES: Cc1cc2cc(n1)-c1cnn(C)c1OCCC[C@@H](C)CN1/C(=N/C2=O)Nc2ccc(C(=O)N(C)C[C@@H]3CCN(CCc4cc(F)c([C@H]5CCC(=O)NC5=O)c(F)c4)CC3(F)F)cc21 ; Cc1cc2cc(n1)-c1cnn(C)c1OCCC[C@@H](C)CN1/C(=N/C2=O)Nc2ccc(C(=O)N(C)C[C@H]3CCN(CCc4cc(F)c([C@H]5CCC(=O)NC5=O)c(F)c4)CC3(F)F)cc21 ; Cc1cc2cc(n1)-c1cnn(C)c1OCCC[C@@H](C)CN1/C(=N/C2=O)Nc2ccc(C(=O)N(C)CC(F)(F)C3CCN(CCc4cc(F)c([C@H]5CCC(=O)NC5=O)c(F)c4)CC3)cc21]

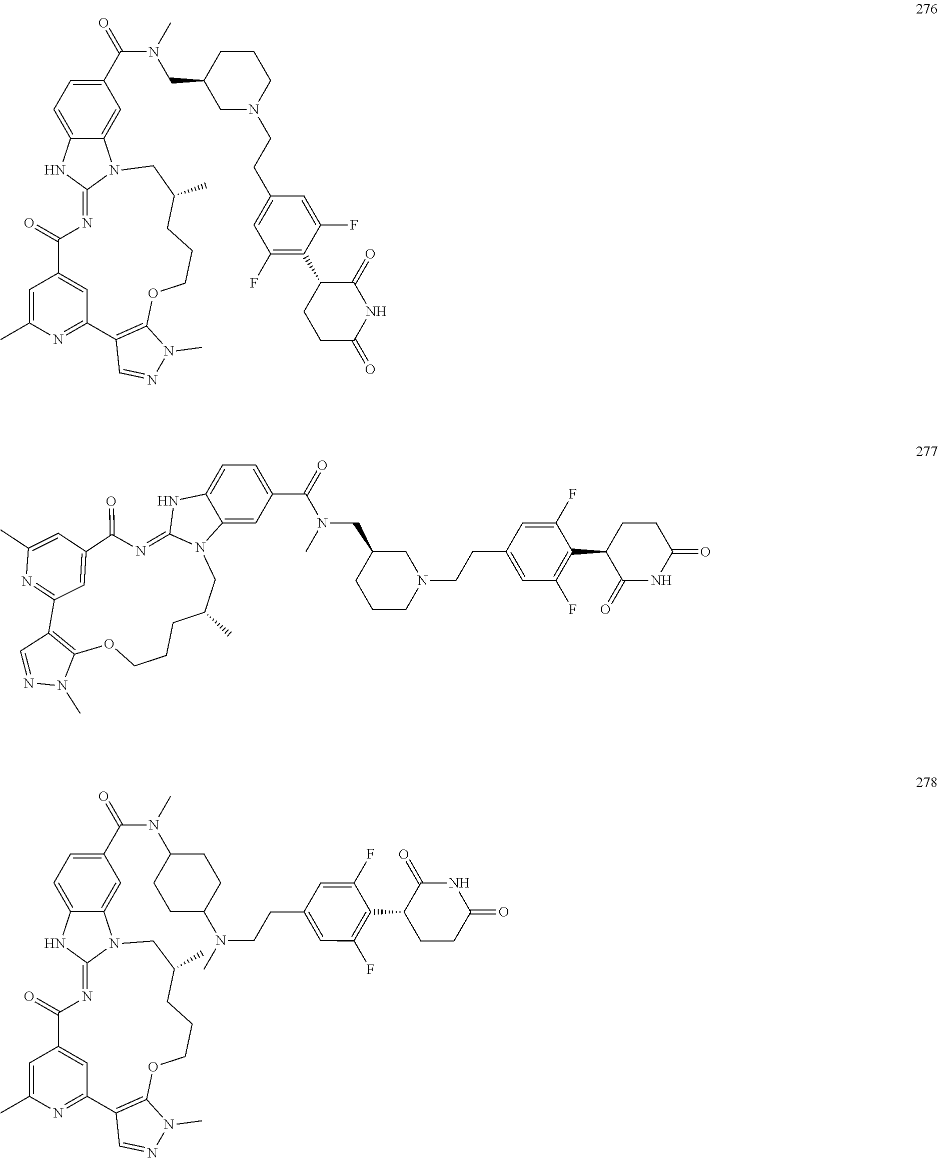 [SMILES: Cc1cc2cc(n1)-c1cnn(C)c1OCCC[C@@H](C)CN1/C(=N/C2=O)Nc2ccc(C(=O)N(C)C[C@H]3CCCN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)C[C@@H]3CCCN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)C3CCC(N(C)CCc4cc(F)c([C@H]5CCC(=O)NC5=O)c(F)c4)CC3)cc21]

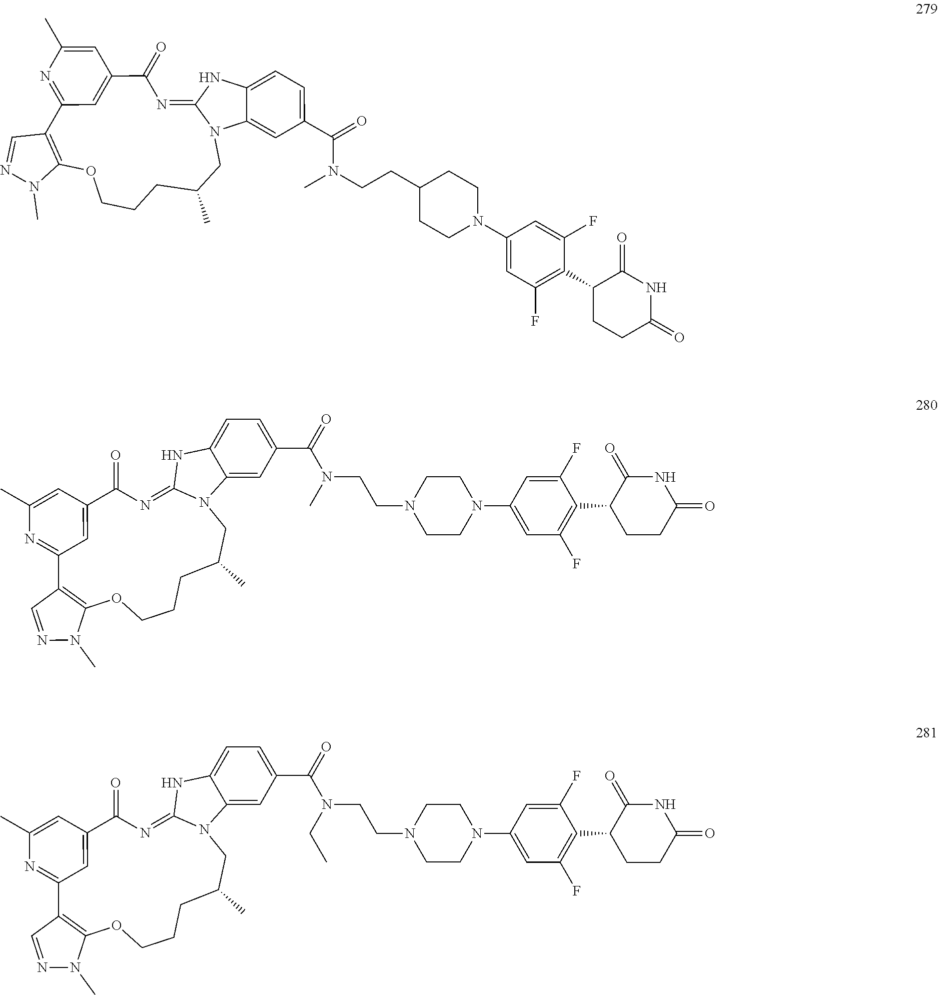 [SMILES: Cc1cc2cc(n1)-c1cnn(C)c1OCCC[C@@H](C)CN1/C(=N/C2=O)Nc2ccc(C(=O)N(C)CCC3CCN(c4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N(C)CCN3CCN(c4cc(F)c([C@H]5CCC(=O)NC5=O)c(F)c4)CC3)cc21 ; CCN(CCN1CCN(c2cc(F)c([C@H]3CCC(=O)NC3=O)c(F)c2)CC1)C(=O)c1ccc2c(c1)N1C[C@H](C)CCCOc3c(cnn3C)-c3cc(cc(C)n3)C(=O)/N=C/1N2]

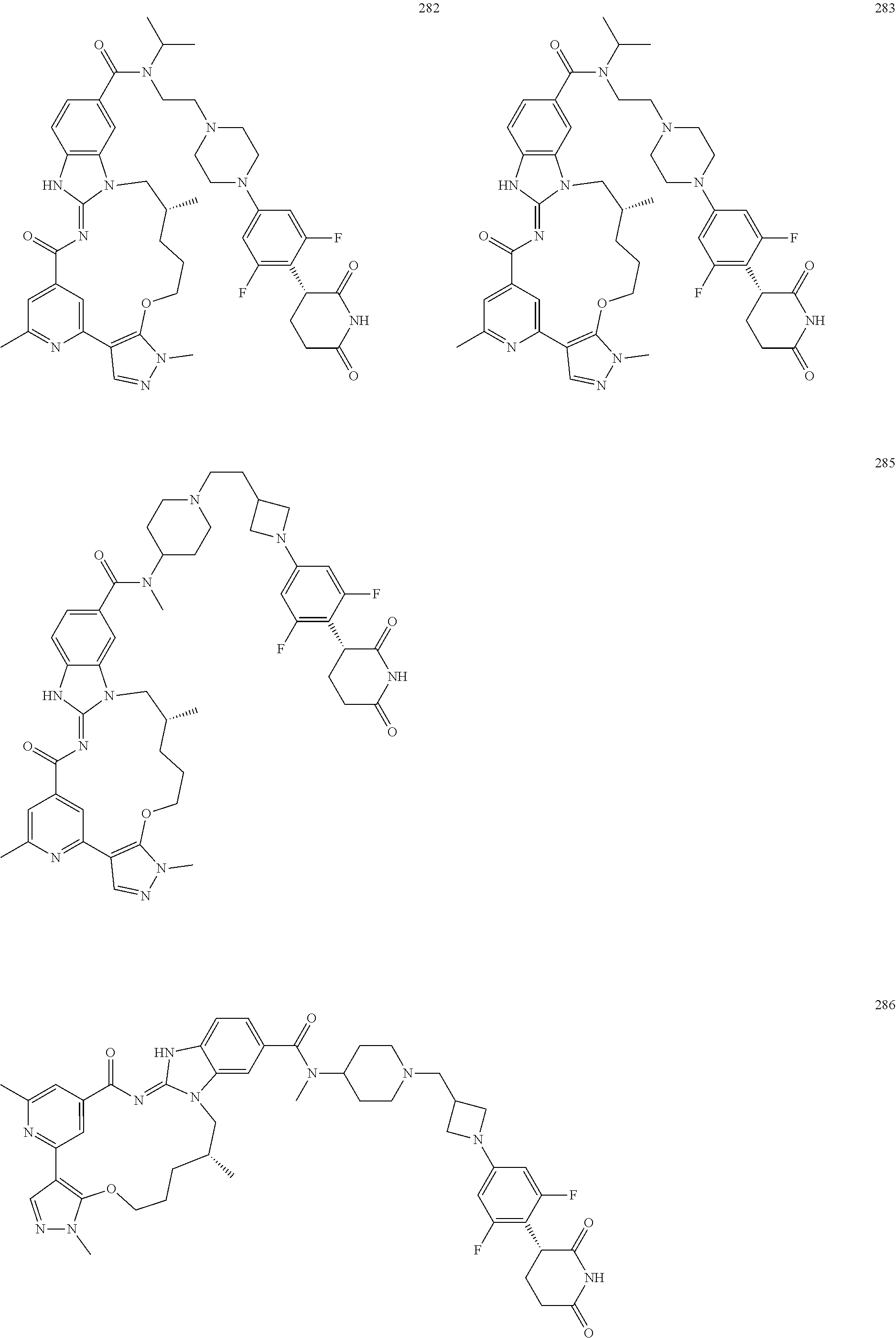 [SMILES: Cc1cc2cc(n1)-c1cnn(C)c1OCCC[C@@H](C)CN1/C(=N/C2=O)Nc2ccc(C(=O)N(CCN3CCN(c4cc(F)c([C@H]5CCC(=O)NC5=O)c(F)c4)CC3)C(C)C)cc21 ; Cc1cc2cc(n1)-c1cnn(C)c1OCCC[C@@H](C)CN1/C(=N/C2=O)Nc2ccc(C(=O)N(CCN3CCN(c4cc(F)c([C@H]5CCC(=O)NC5=O)c(F)c4)CC3)C(C)C)cc21 ; Cc1cc2cc(n1)-c1cnn(C)c1OCCC[C@@H](C)CN1/C(=N/C2=O)Nc2ccc(C(=O)N(C)C3CCN(CCC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C(=O)N(C)C3CCN(CC4CN(c5cc(F)c([C@H]6CCC(=O)NC6=O)c(F)c5)C4)CC3)cc21]

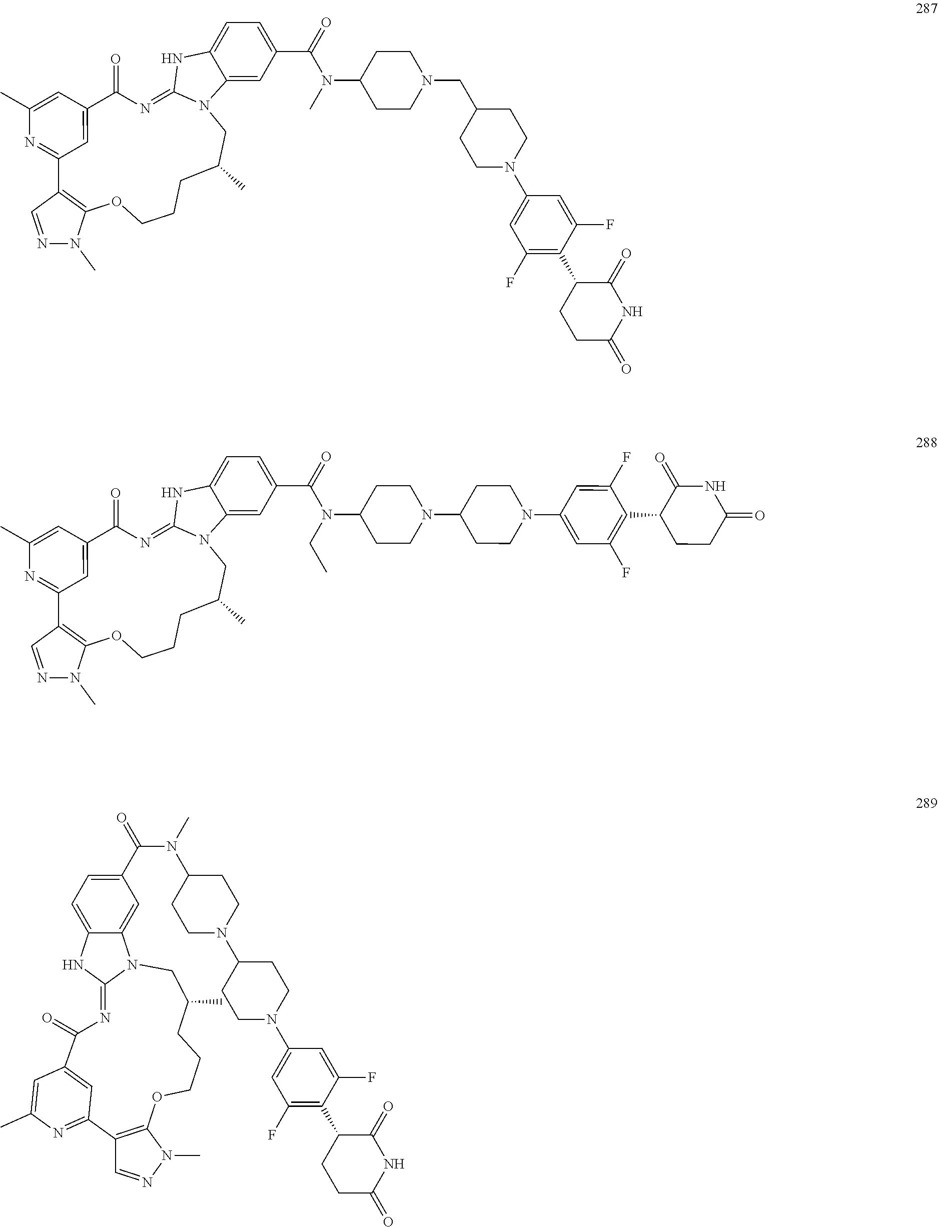 [SMILES: Cc1cc2cc(n1)-c1cnn(C)c1OCCC[C@@H](C)CN1/C(=N/C2=O)Nc2ccc(C(=O)N(C)C3CCN(CC4CCN(c5cc(F)c([C@H]6CCC(=O)NC6=O)c(F)c5)CC4)CC3)cc21 ; CCN(C(=O)c1ccc2c(c1)N1C[C@H](C)CCCOc3c(cnn3C)-c3cc(cc(C)n3)C(=O)/N=C/1N2)C1CCN(C2CCN(c3cc(F)c([C@H]4CCC(=O)NC4=O)c(F)c3)CC2)CC1 ; Cc1cc2cc(n1)-c1cnn(C)c1OCCC[C@@H](C)CN1/C(=N/C2=O)Nc2ccc(C(=O)N(C)C3CCN(C4CCN(c5cc(F)c([C@H]6CCC(=O)NC6=O)c(F)c5)CC4)CC3)cc21]

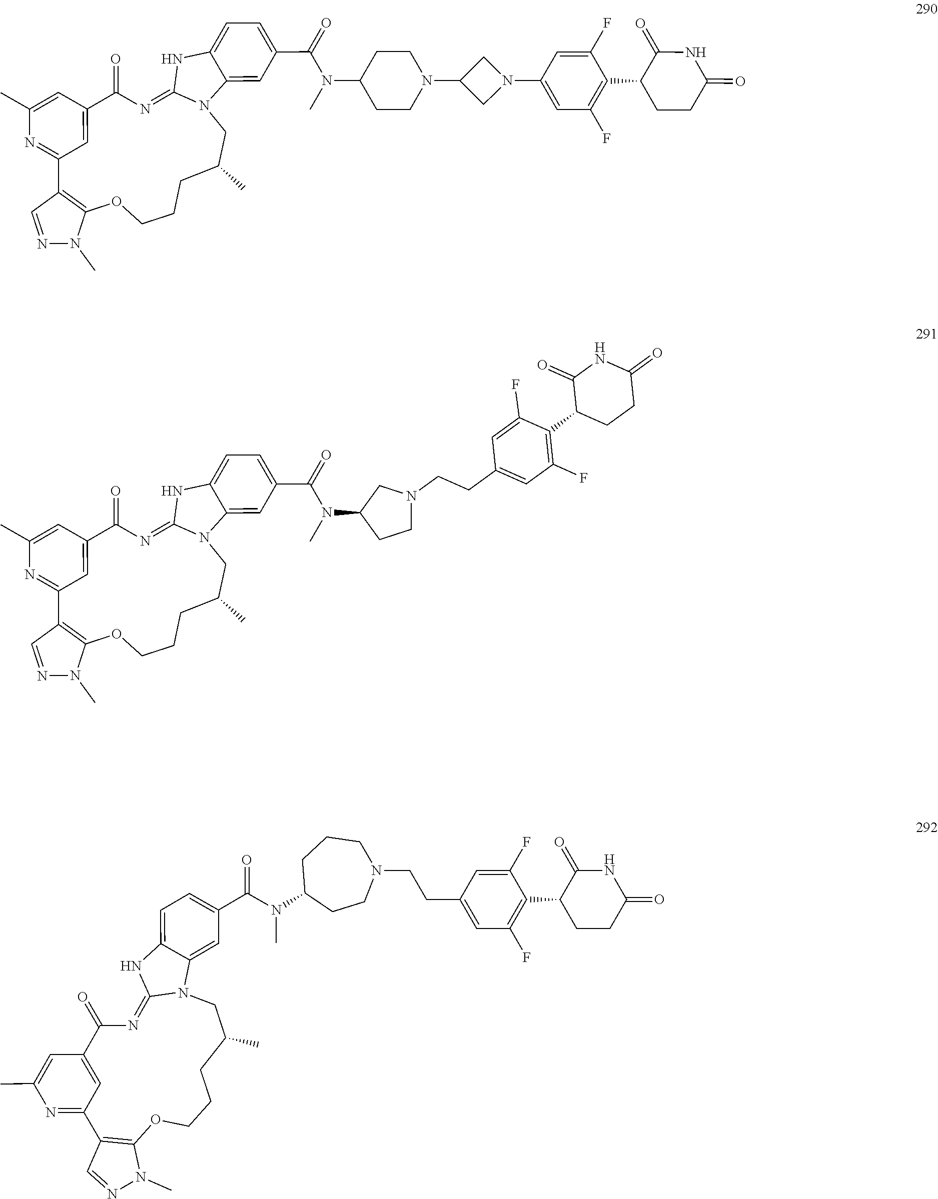 [SMILES: Cc1cc2cc(n1)-c1cnn(C)c1OCCC[C@@H](C)CN1/C(=N/C2=O)Nc2ccc(C(=O)N(C)C3CCN(C4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C(=O)N(C)[C@@H]3CCN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)[C@@H]3CCCN(CCc4cc(F)c([C@H]5CCC(=O)NC5=O)c(F)c4)CC3)cc21]

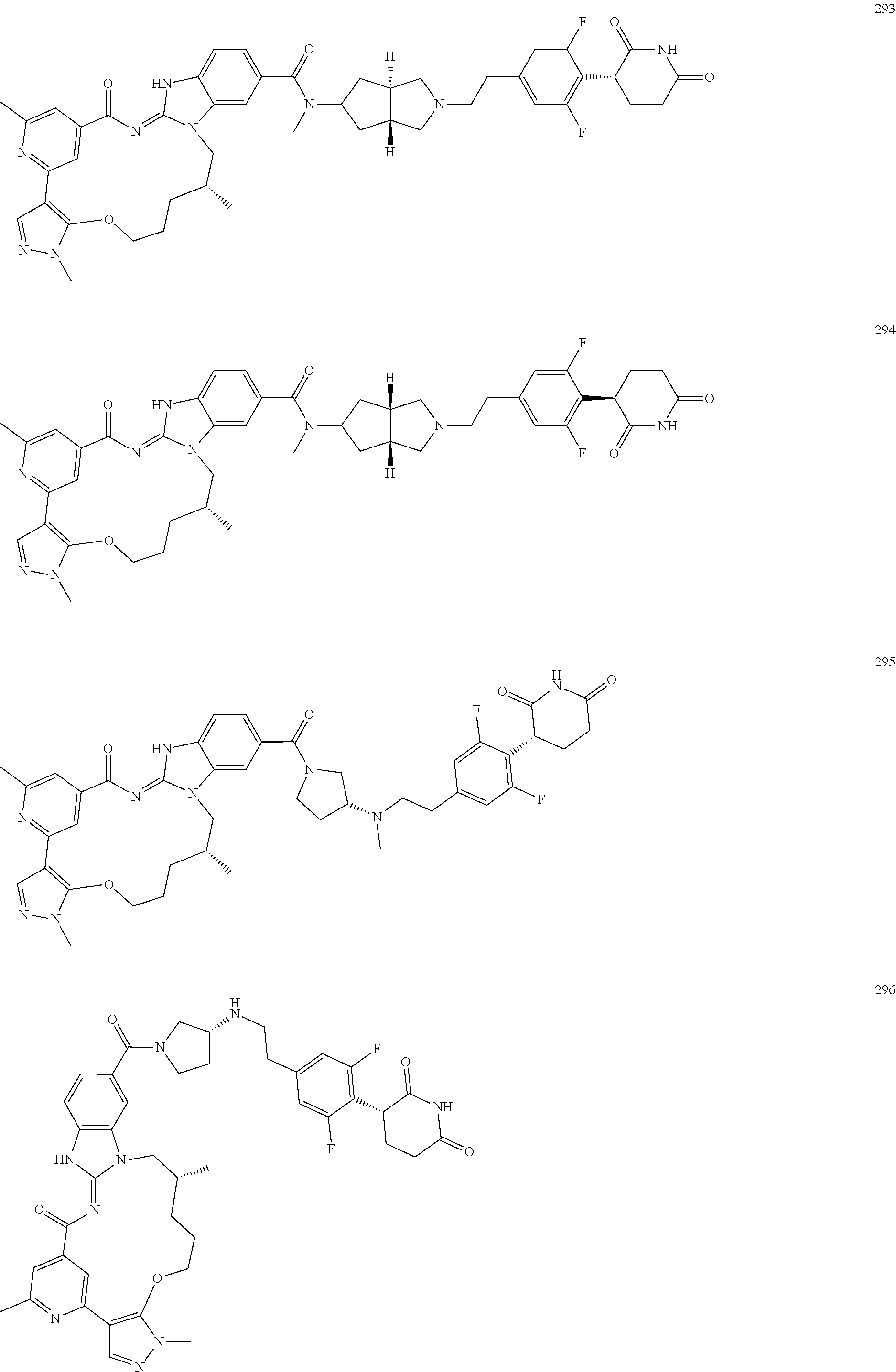

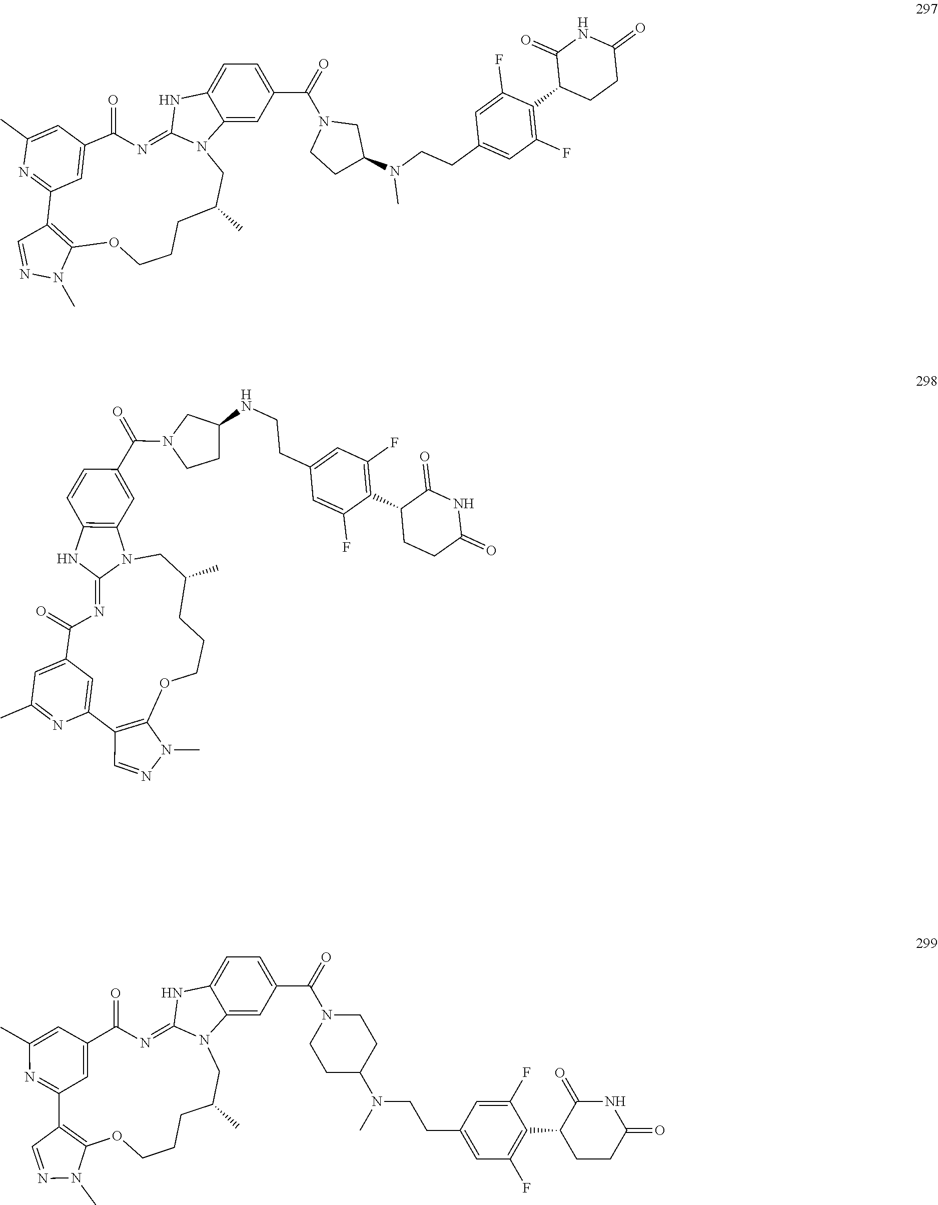 [SMILES: Cc1cc2cc(n1)-c1cnn(C)c1OCCC[C@@H](C)CN1/C(=N/C2=O)Nc2ccc(C(=O)N3CC[C@H](N(C)CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N3CC[C@H](NCCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N3CCC(N(C)CCc4cc(F)c([C@H]5CCC(=O)NC5=O)c(F)c4)CC3)cc21]

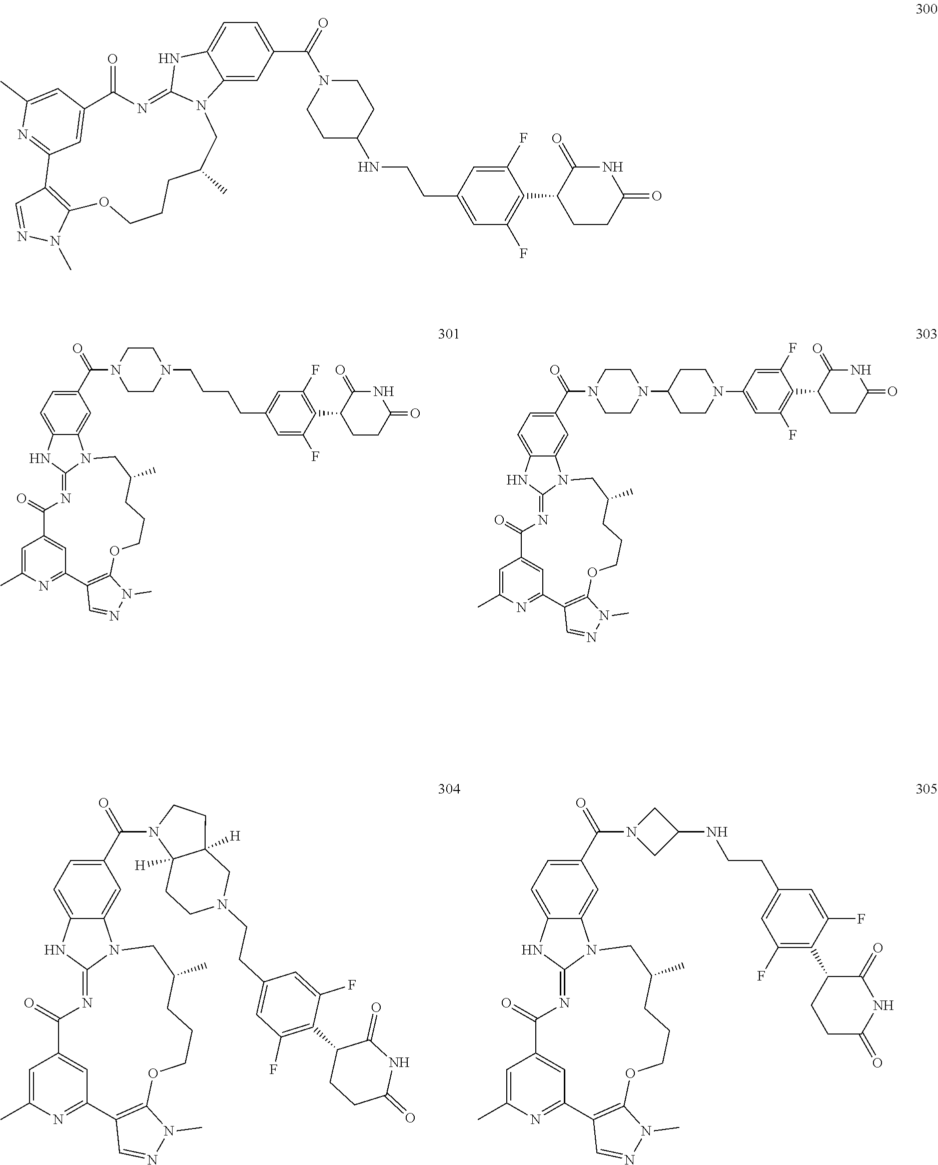

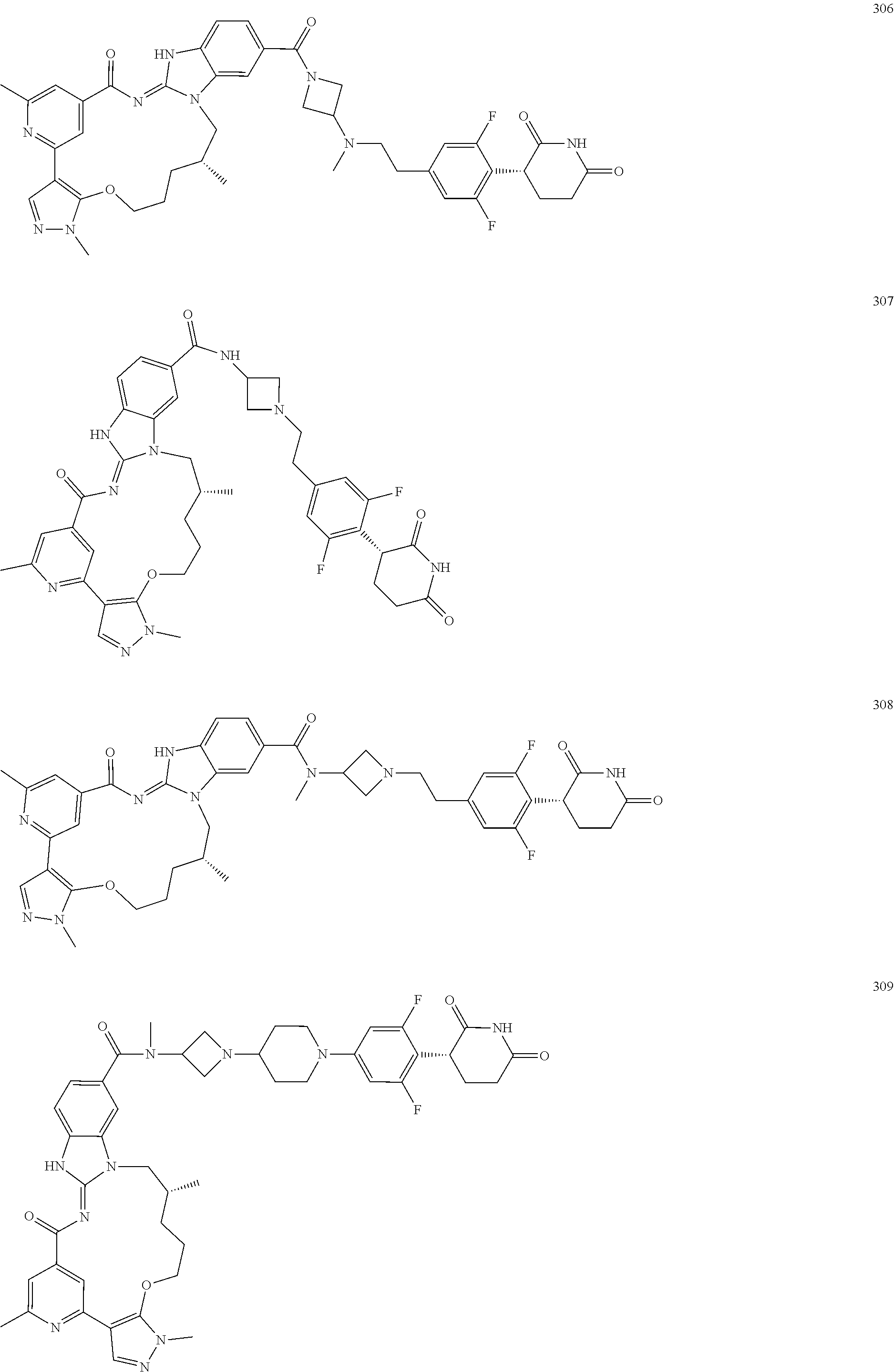 [SMILES: Cc1cc2cc(n1)-c1cnn(C)c1OCCC[C@@H](C)CN1/C(=N/C2=O)Nc2ccc(C(=O)N3CC(N(C)CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)NC3CN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)C3CN(CCc4cc(F)c([C@H]5CCC(=O)NC5=O)c(F)c4)C3)cc21 ; Cc1cc2cc(n1)-c1cnn(C)c1OCCC[C@@H](C)CN1/C(=N/C2=O)Nc2ccc(C(=O)N(C)C3CN(C4CCN(c5cc(F)c([C@H]6CCC(=O)NC6=O)c(F)c5)CC4)C3)cc21]

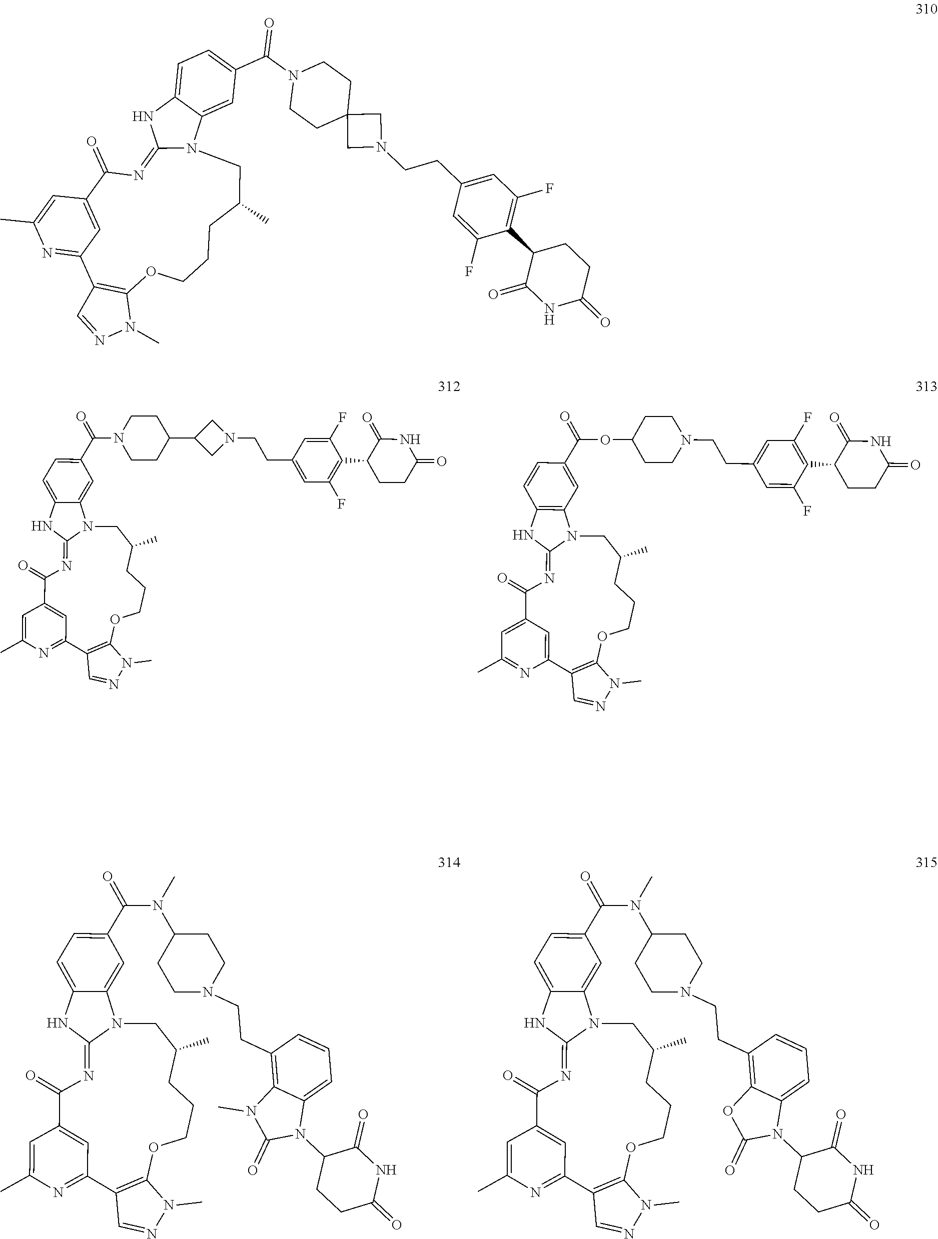

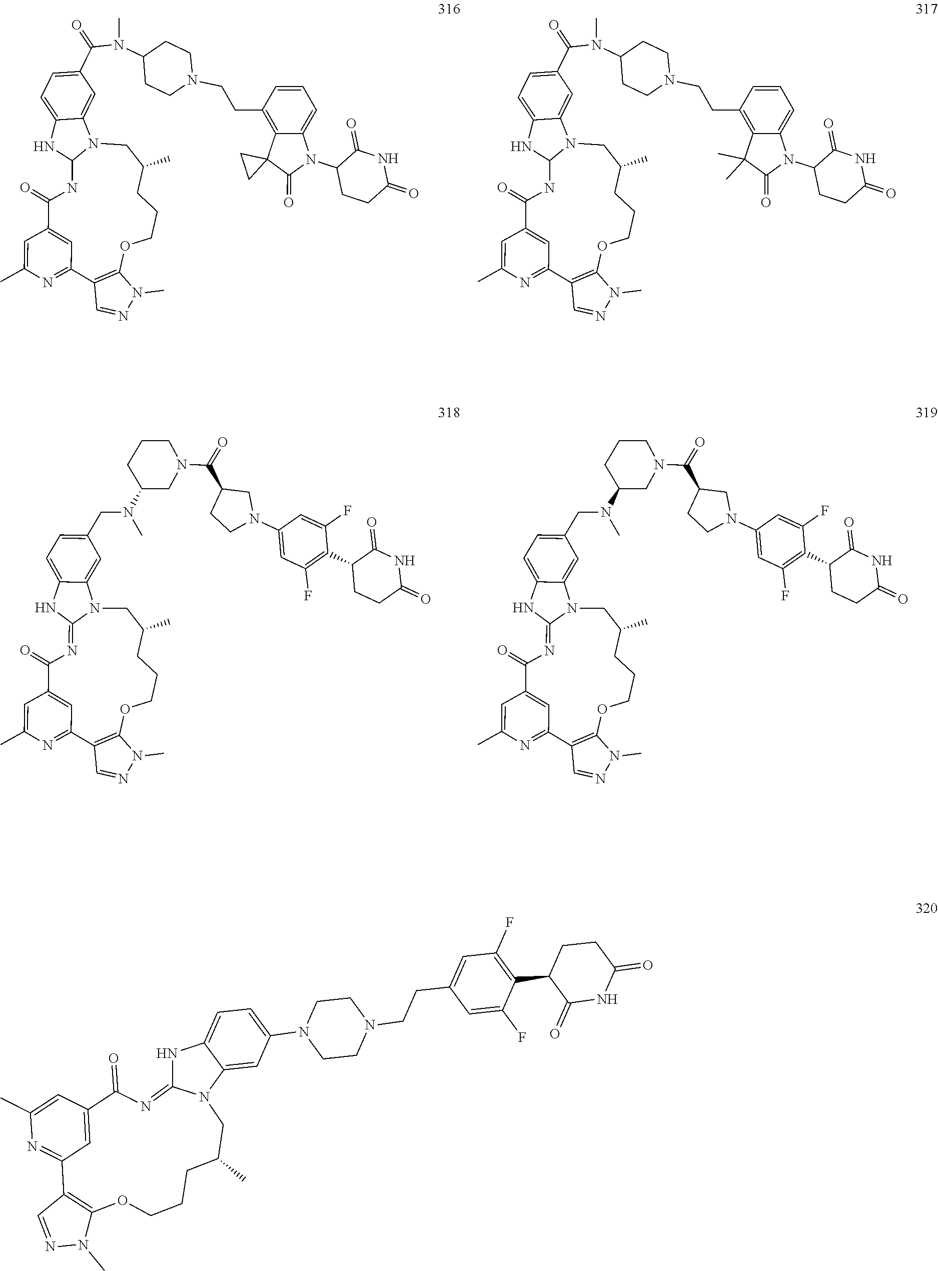

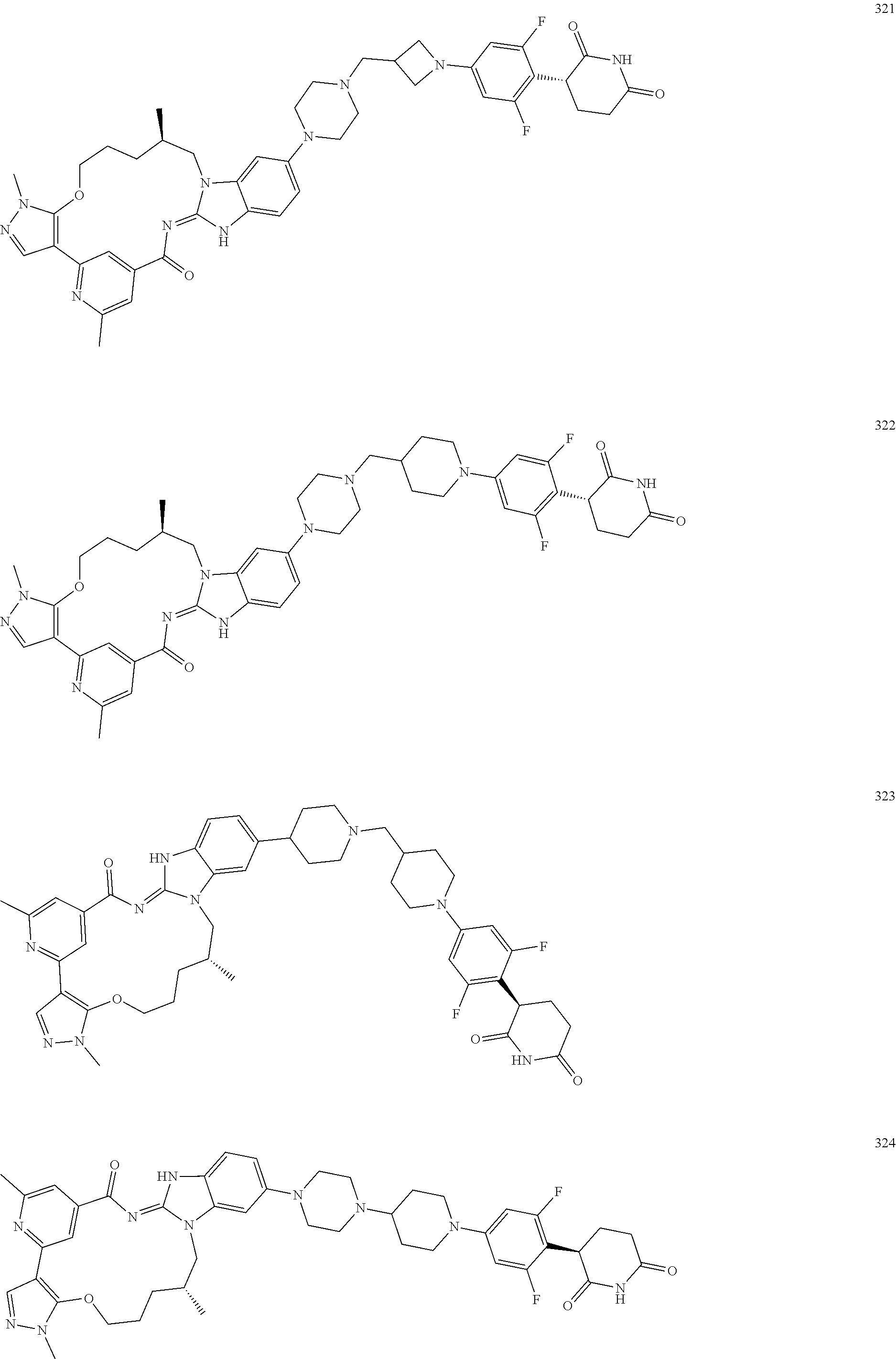 [SMILES: Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(N3CCN(CC4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(C3CCN(CC4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21]

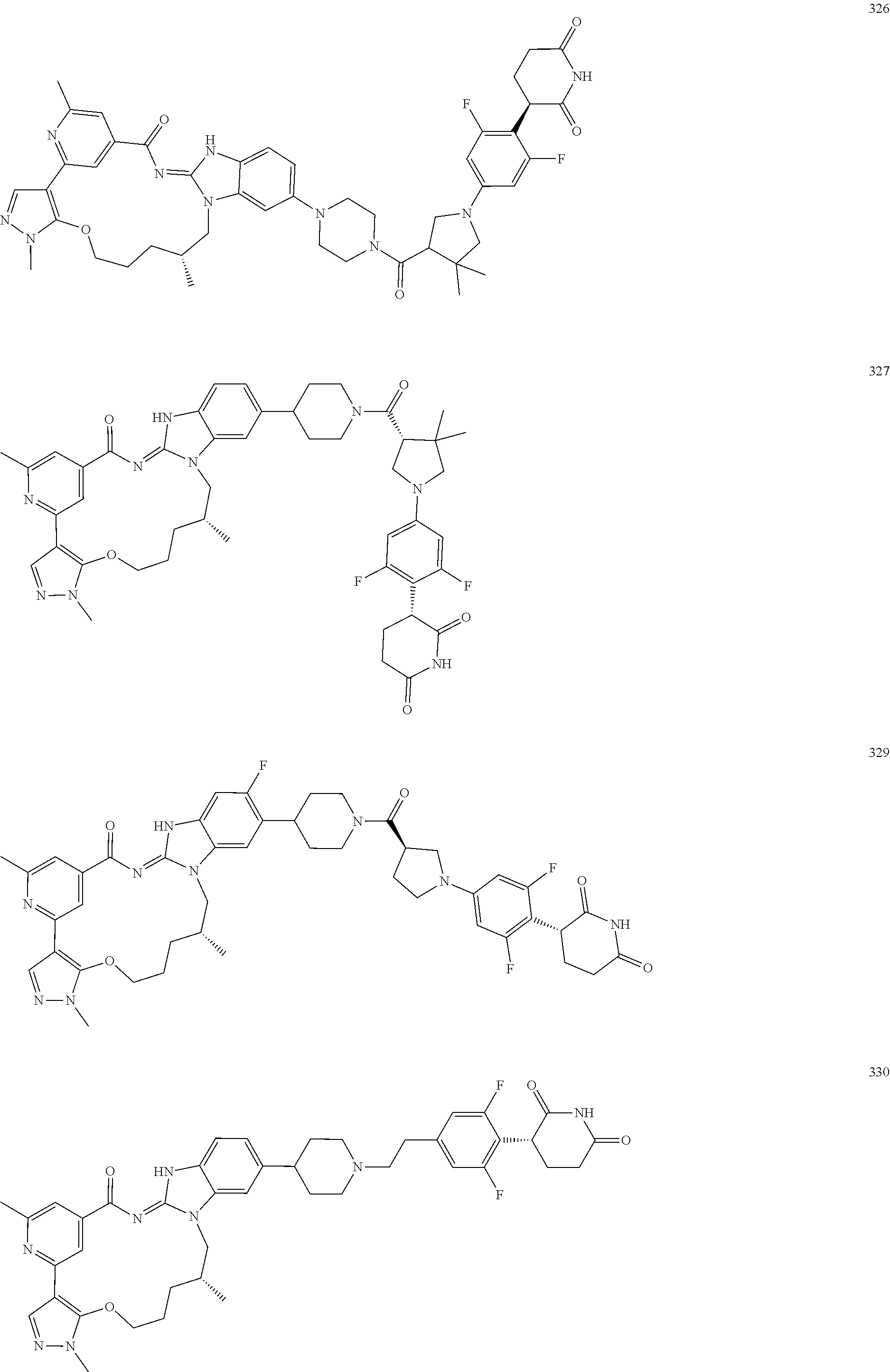 [SMILES: Cc1cc2cc(n1)-c1cnn(C)c1OCCC[C@@H](C)CN1/C(=N/C2=O)Nc2ccc(N3CCN(C(=O)C4CN(c5cc(F)c([C@H]6CCC(=O)NC6=O)c(F)c5)CC4(C)C)CC3)cc21 ; Cc1cc2cc(n1)-c1cnn(C)c1OCCC[C@@H](C)CN1/C(=N/C2=O)Nc2ccc(C3CCN(C(=O)[C@H]4CN(c5cc(F)c([C@H]6CCC(=O)NC6=O)c(F)c5)CC4(C)C)CC3)cc21 ; Cc1cc2cc(n1)-c1cnn(C)c1OCCC[C@@H](C)CN1/C(=N/C2=O)Nc2cc(F)c(C3CCN(C(=O)[C@@H]4CCN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C3CCN(CCc4cc(F)c([C@H]5CCC(=O)NC5=O)c(F)c4)CC3)cc21]

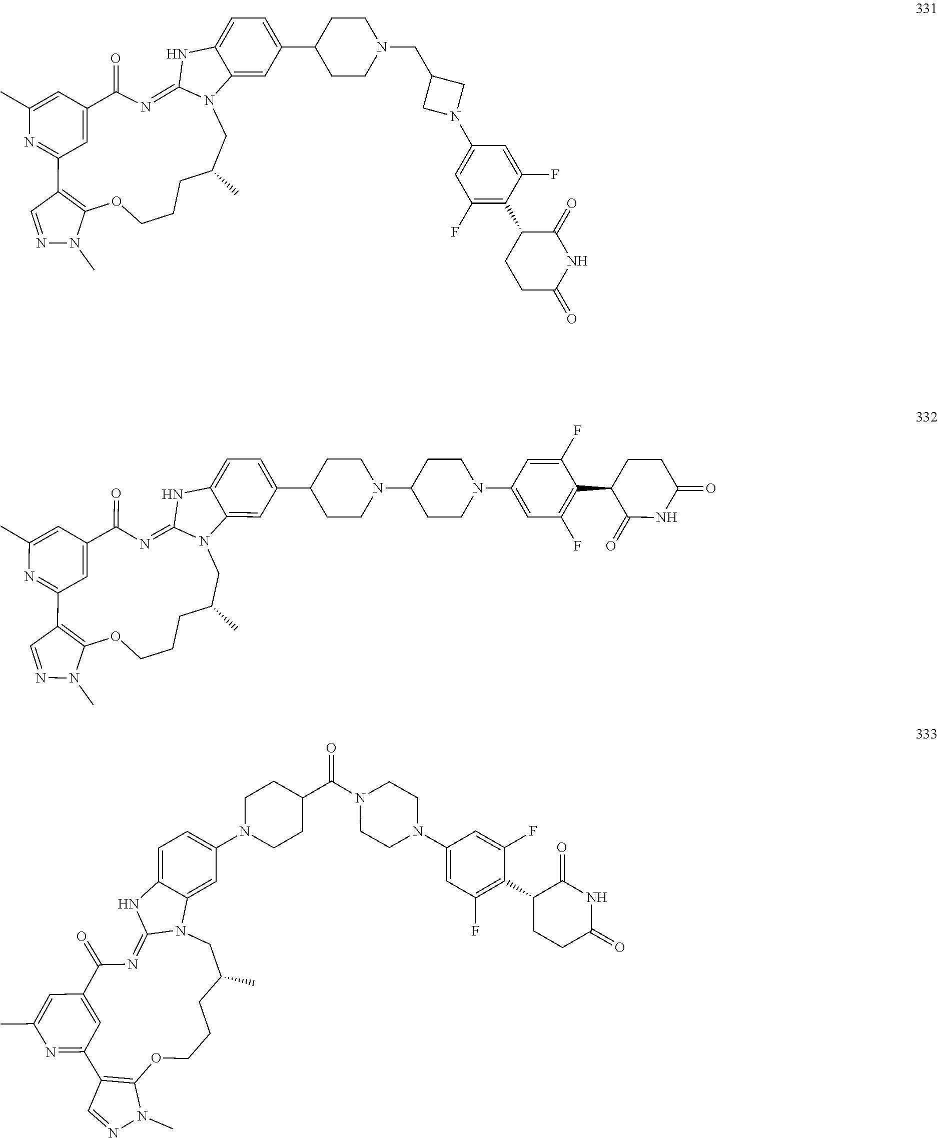 [SMILES: Cc1cc2cc(n1)-c1cnn(C)c1OCCC[C@@H](C)CN1/C(=N/C2=O)Nc2ccc(C3CCN(CC4CN(c5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(C3CCN(C4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCC(C(=O)N4CCN(c5cc(F)c([C@H]6CCC(=O)NC6=O)c(F)c5)CC4)CC3)cc21]

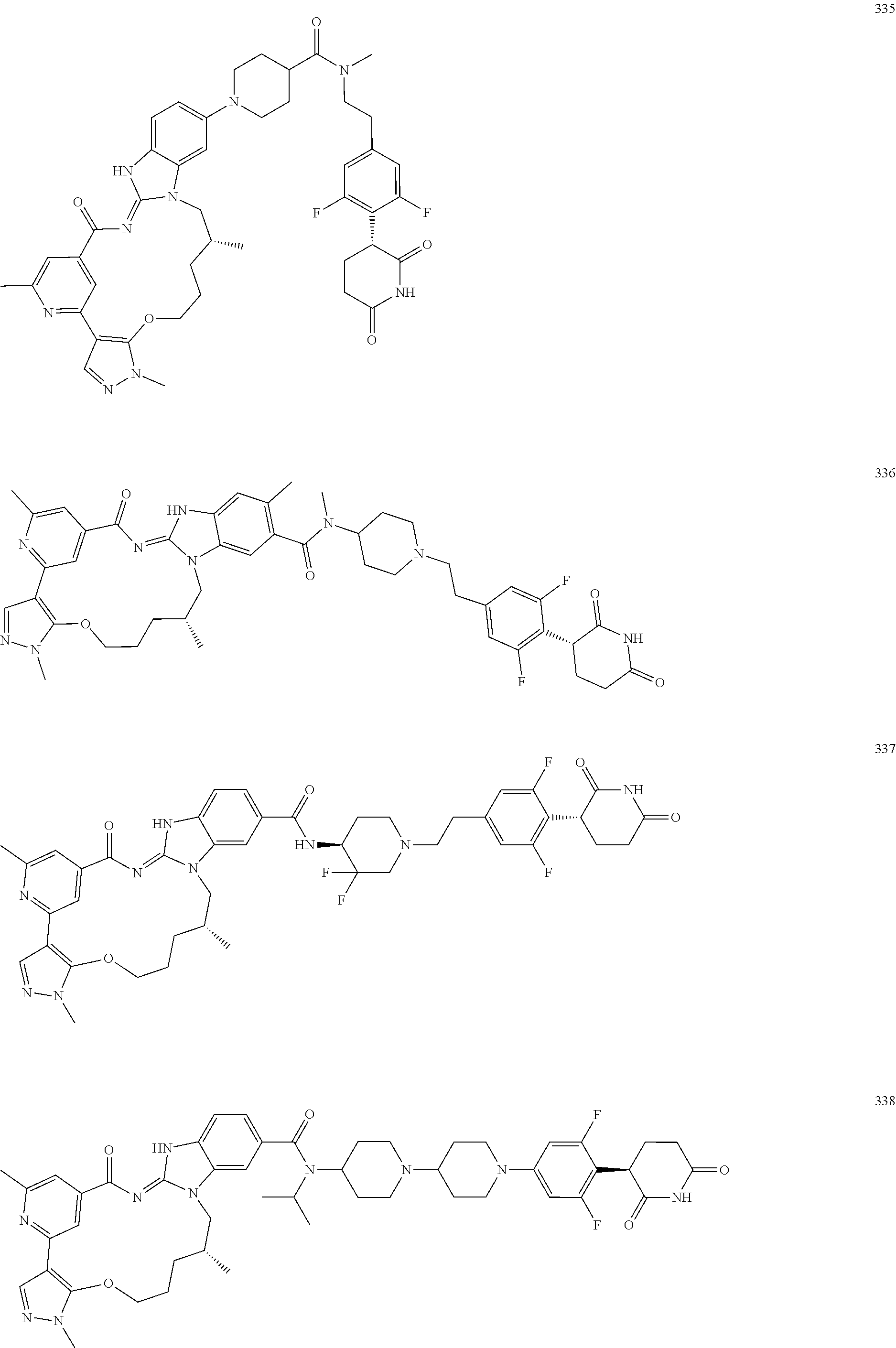 [SMILES: Cc1cc2cc(n1)-c1cnn(C)c1OCCC[C@@H](C)CN1/C(=N/C2=O)Nc2ccc(N3CCC(C(=O)N(C)CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2cc(C)c(C(=O)N(C)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)CC3(F)F)cc21 ; Cc1cc2cc(n1)-c1cnn(C)c1OCCC[C@@H](C)CN1/C(=N/C2=O)Nc2ccc(C(=O)N(C(C)C)C3CCN(C4CCN(c5cc(F)c([C@H]6CCC(=O)NC6=O)c(F)c5)CC4)CC3)cc21]

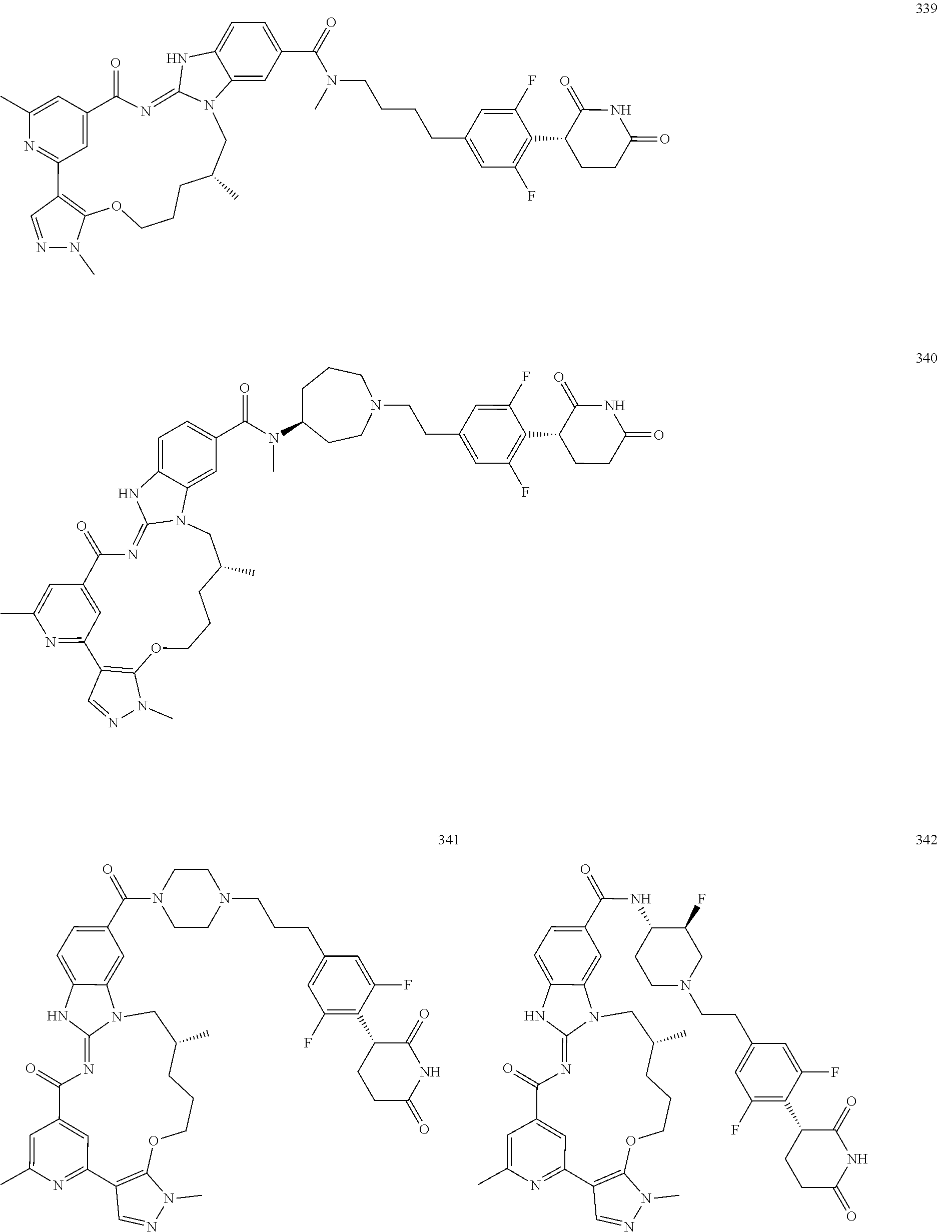 [SMILES: Cc1cc2cc(n1)-c1cnn(C)c1OCCC[C@@H](C)CN1/C(=N/C2=O)Nc2ccc(C(=O)N(C)CCCCc3cc(F)c([C@H]4CCC(=O)NC4=O)c(F)c3)cc21 ; Cc1cc2cc(n1)-c1cnn(C)c1OCCC[C@@H](C)CN1/C(=N/C2=O)Nc2ccc(C(=O)N(C)[C@H]3CCCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N3CCN(CCCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C(=O)N[C@H]3CCN(CCc4cc(F)c([C@H]5CCC(=O)NC5=O)c(F)c4)C[C@@H]3F)cc21]

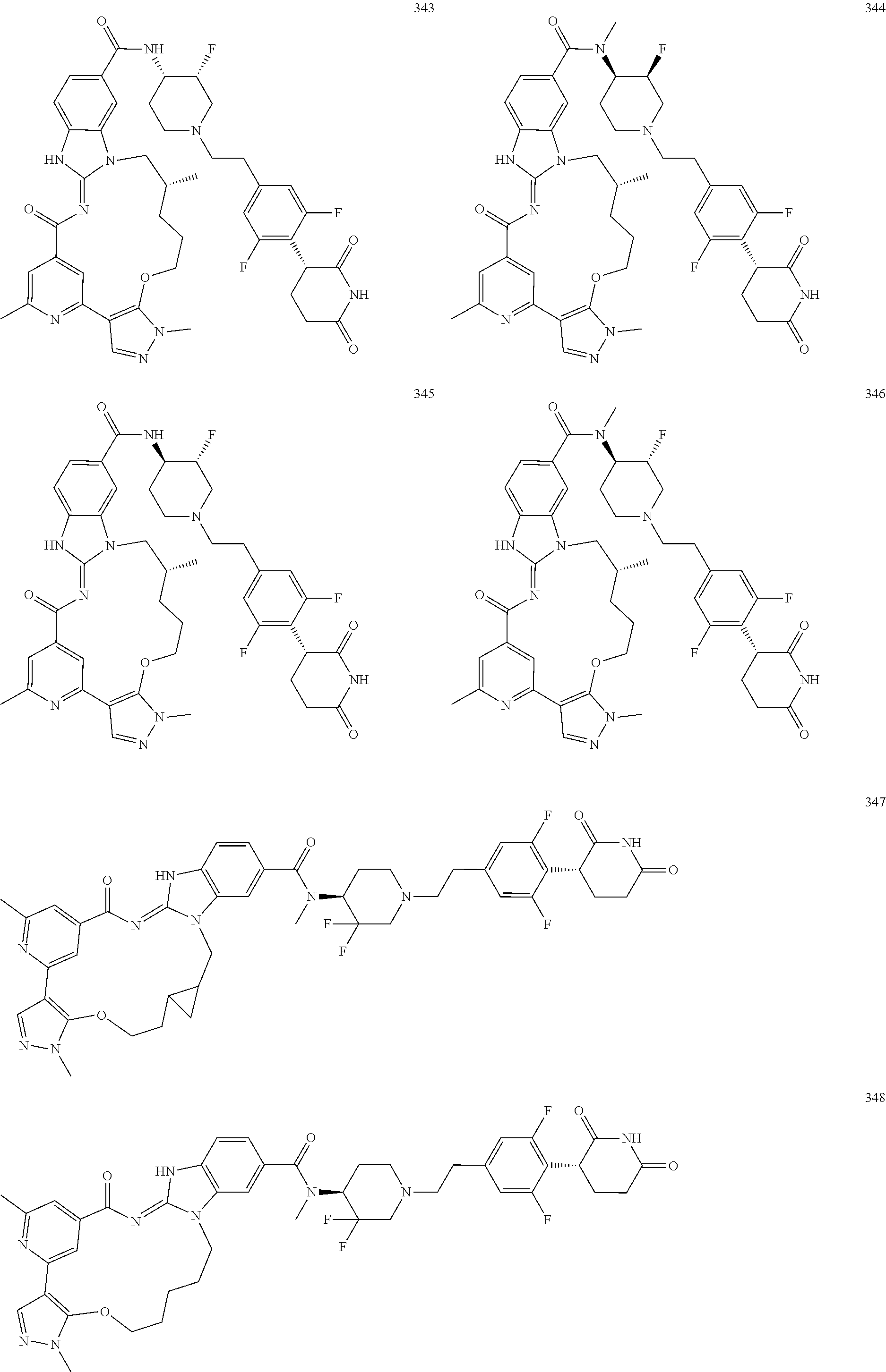

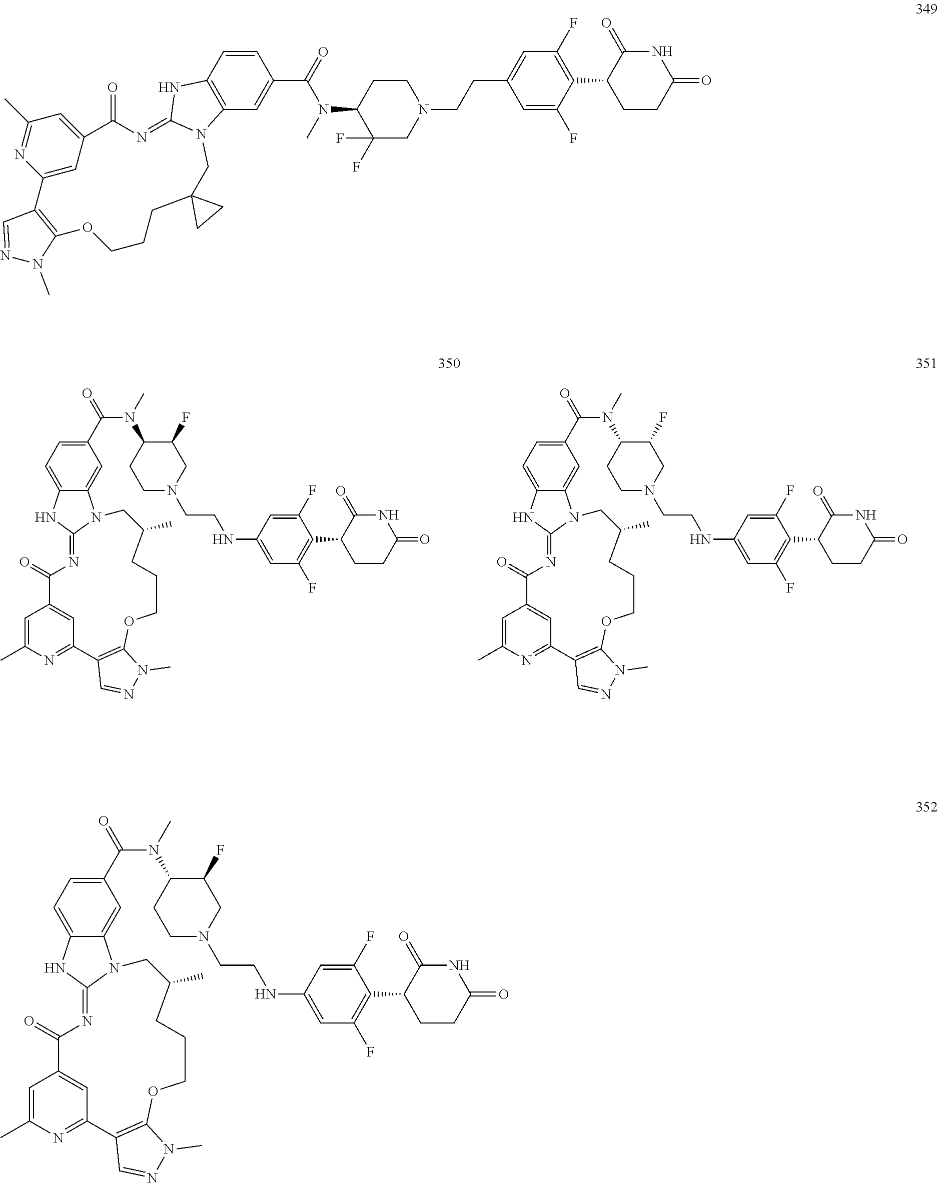

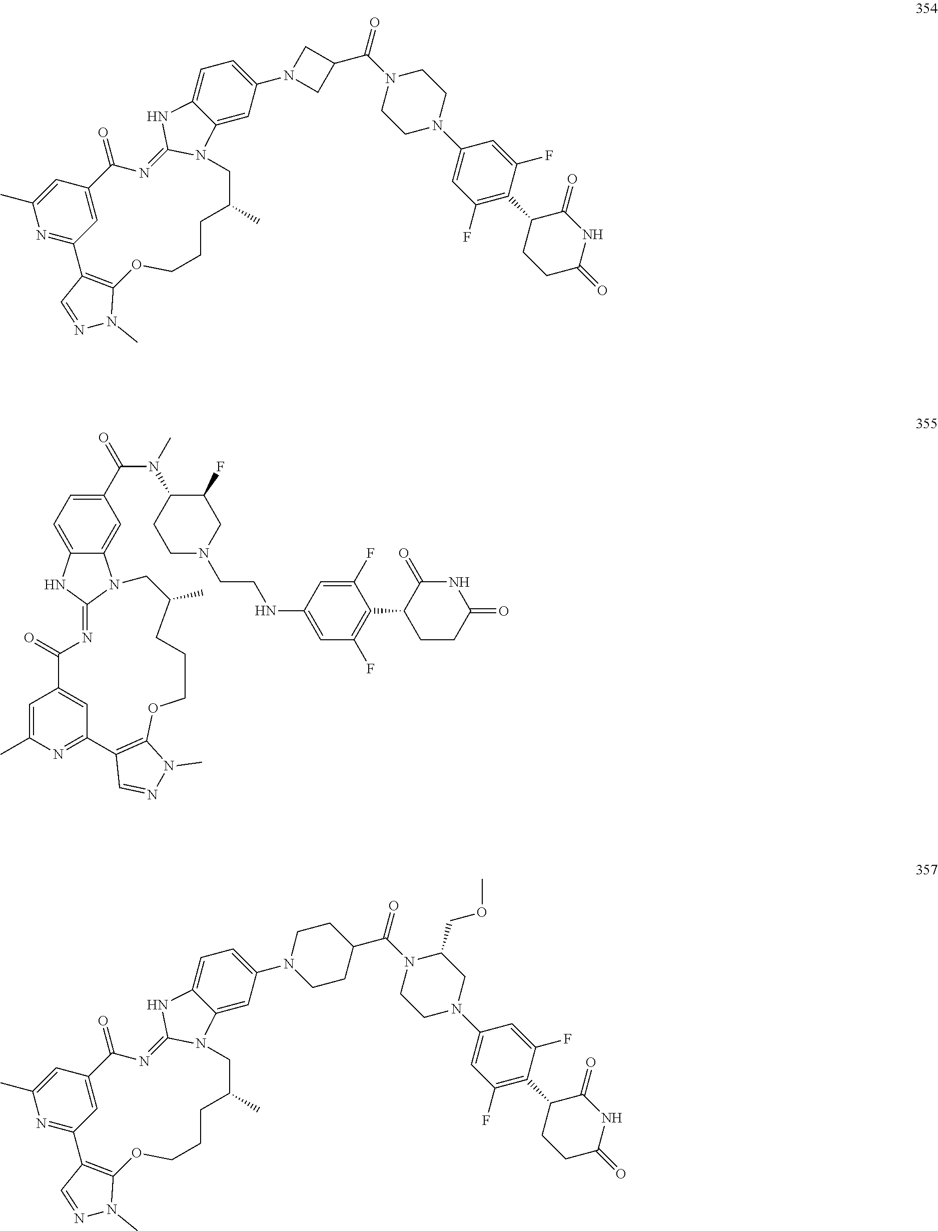 [SMILES: Cc1cc2cc(n1)-c1cnn(C)c1OCCC[C@@H](C)CN1/C(=N/C2=O)Nc2ccc(N3CC(C(=O)N4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(C(=O)N(C)[C@H]3CCN(CCNc4cc(F)c([C@H]5CCC(=O)NC5=O)c(F)c4)C[C@@H]3F)cc21 ; COC[C@@H]1CN(c2cc(F)c([C@H]3CCC(=O)NC3=O)c(F)c2)CCN1C(=O)C1CCN(c2ccc3c(c2)N2C[C@H](C)CCCOc4c(cnn4C)-c4cc(cc(C)n4)C(=O)/N=C/2N3)CC1]

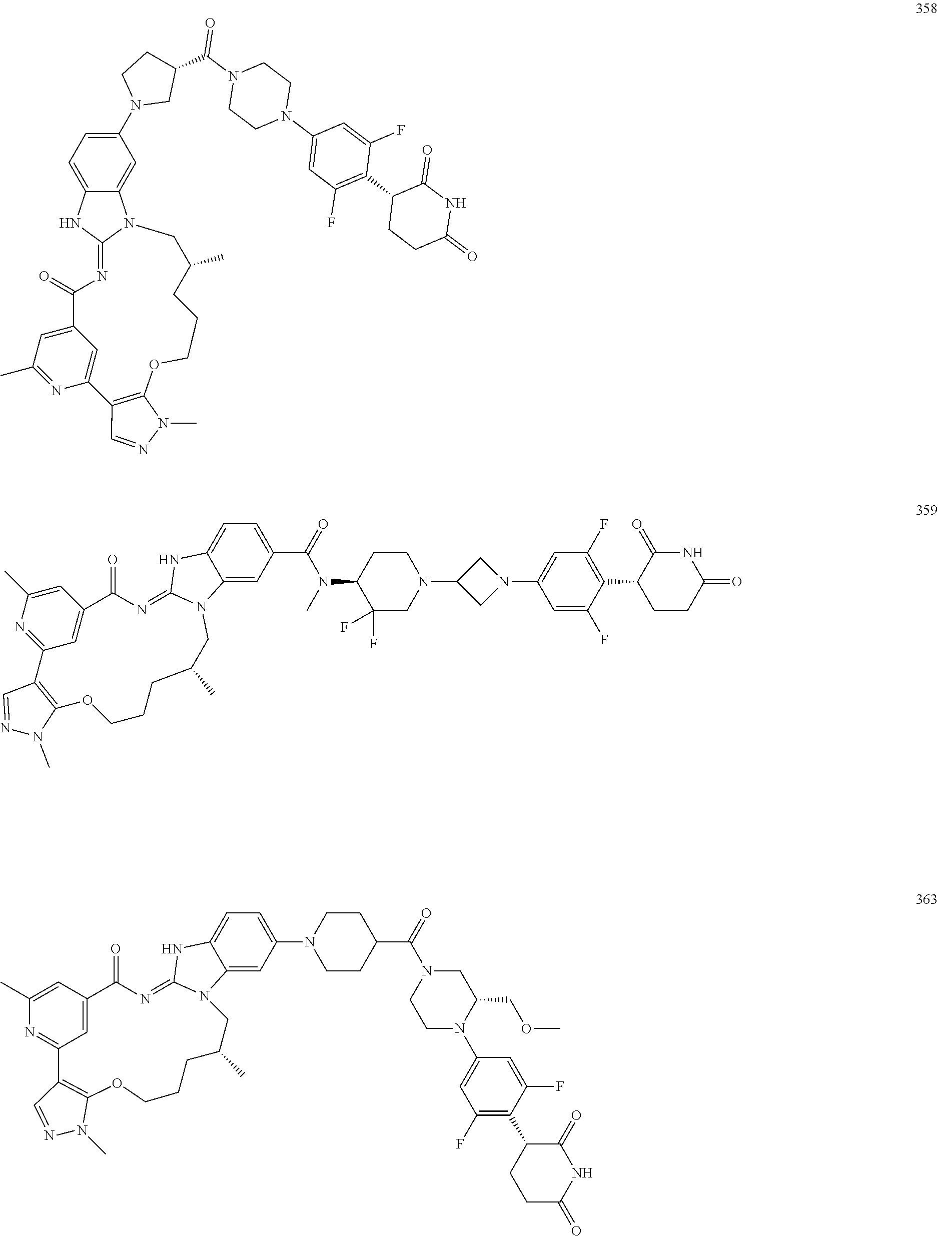 [SMILES: Cc1cc2cc(n1)-c1cnn(C)c1OCCC[C@@H](C)CN1/C(=N/C2=O)Nc2ccc(N3CC[C@H](C(=O)N4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(C(=O)N(C)[C@H]3CCN(C4CN(c5cc(F)c([C@H]6CCC(=O)NC6=O)c(F)c5)C4)CC3(F)F)cc21 ; COC[C@H]1CN(C(=O)C2CCN(c3ccc4c(c3)N3C[C@H](C)CCCOc5c(cnn5C)-c5cc(cc(C)n5)C(=O)/N=C/3N4)CC2)CCN1c1cc(F)c([C@H]2CCC(=O)NC2=O)c(F)c1]

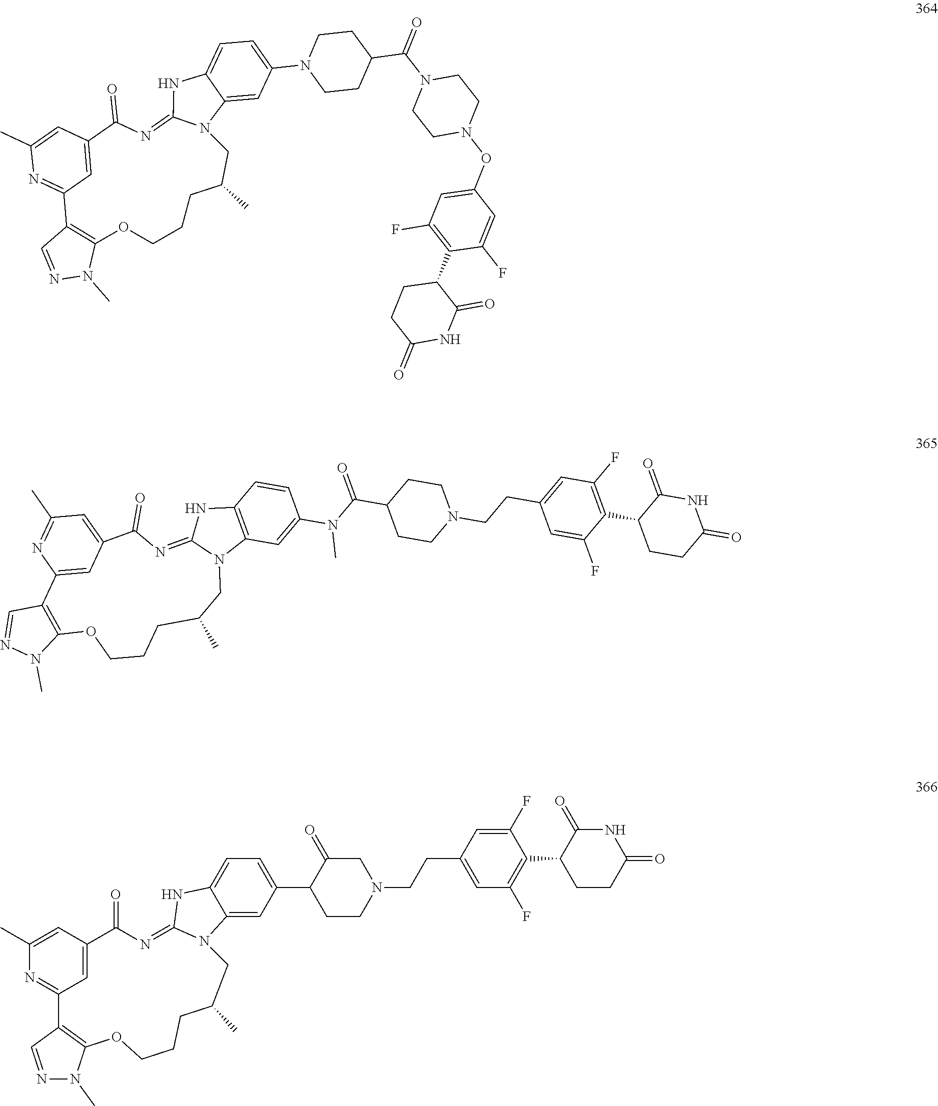 [SMILES: Cc1cc2cc(n1)-c1cnn(C)c1OCCC[C@@H](C)CN1/C(=N/C2=O)Nc2ccc(N3CCC(C(=O)N4CCN(Oc5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N(C)C(=O)C3CCN(CCc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(C3CCN(CCc4cc(F)c([C@H]5CCC(=O)NC5=O)c(F)c4)CC3=O)cc21]

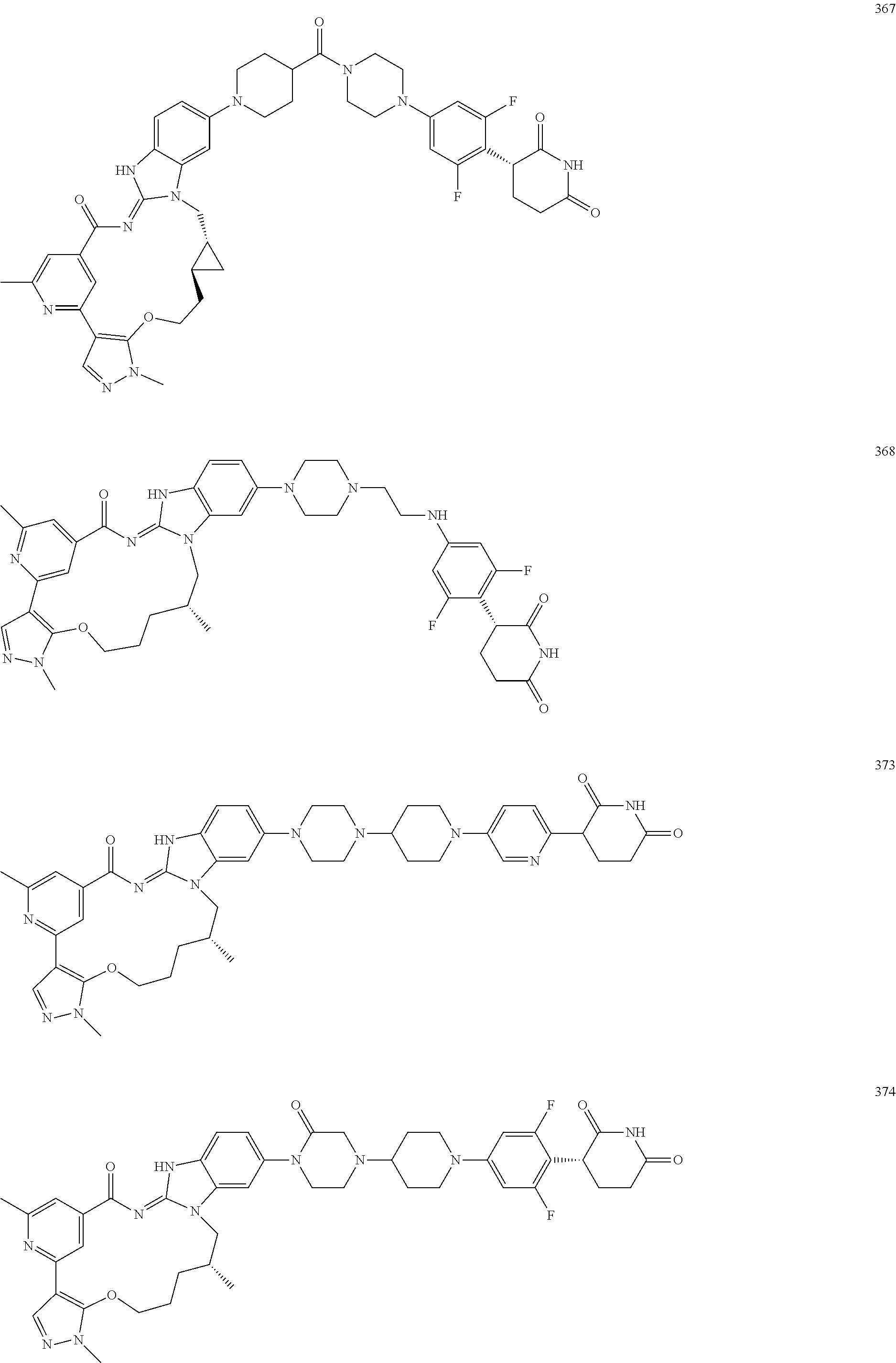 [SMILES: Cc1cc2cc(n1)-c1cnn(C)c1OCC[C@@H]1C[C@H]1CN1/C(=N/C2=O)Nc2ccc(N3CCC(C(=O)N4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCN(CCNc4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5)CC4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3=O)cc21]

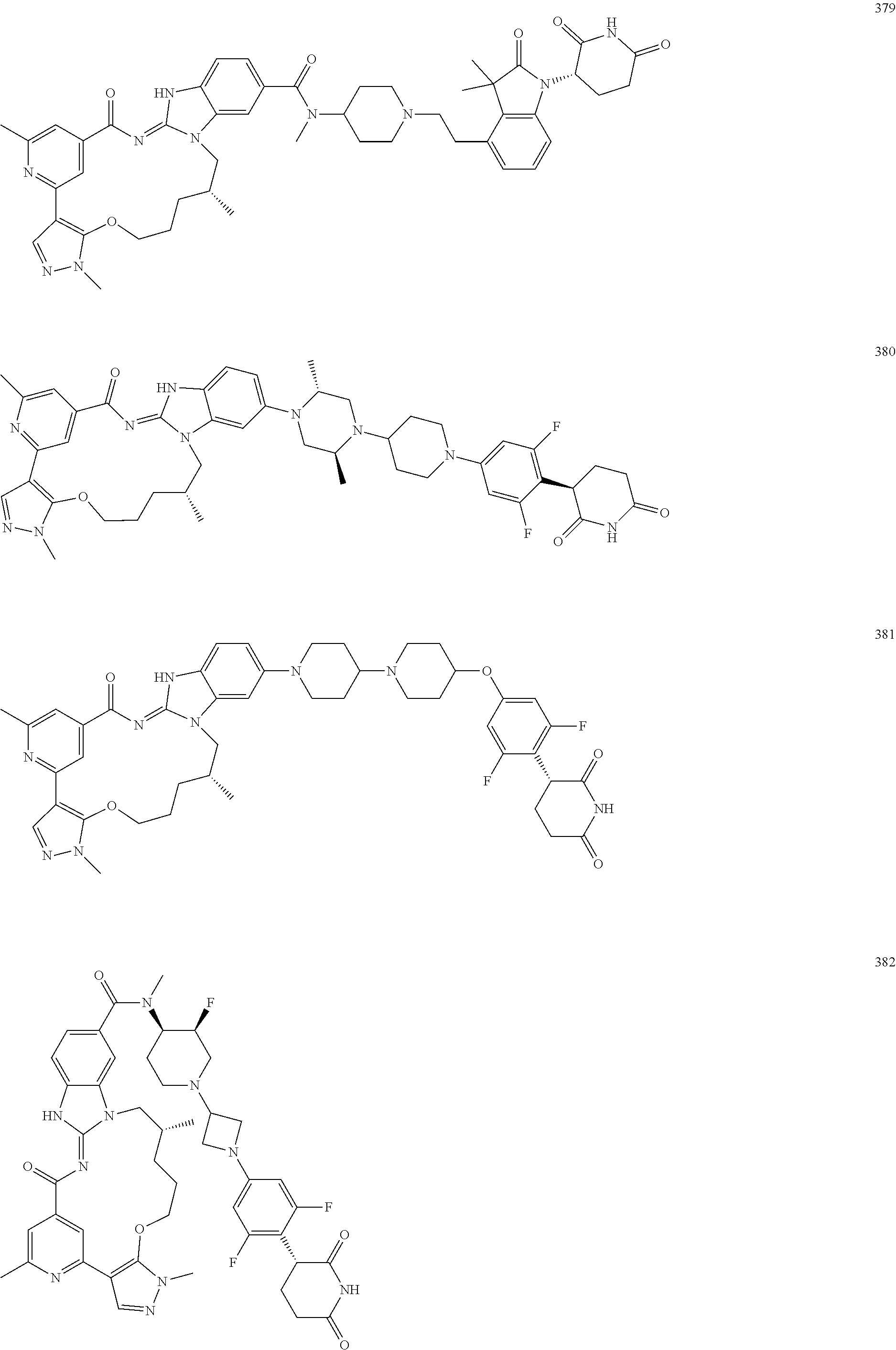

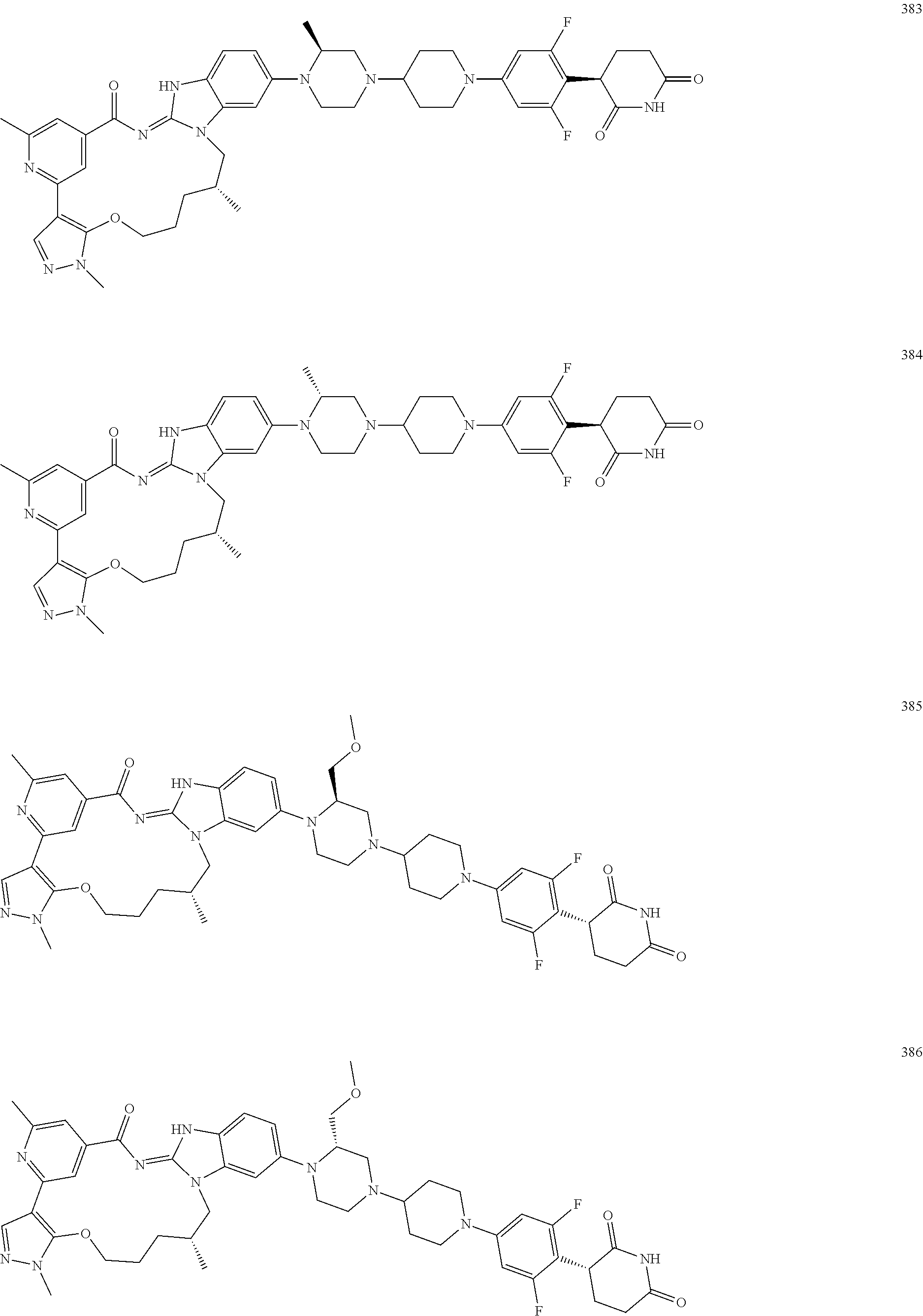

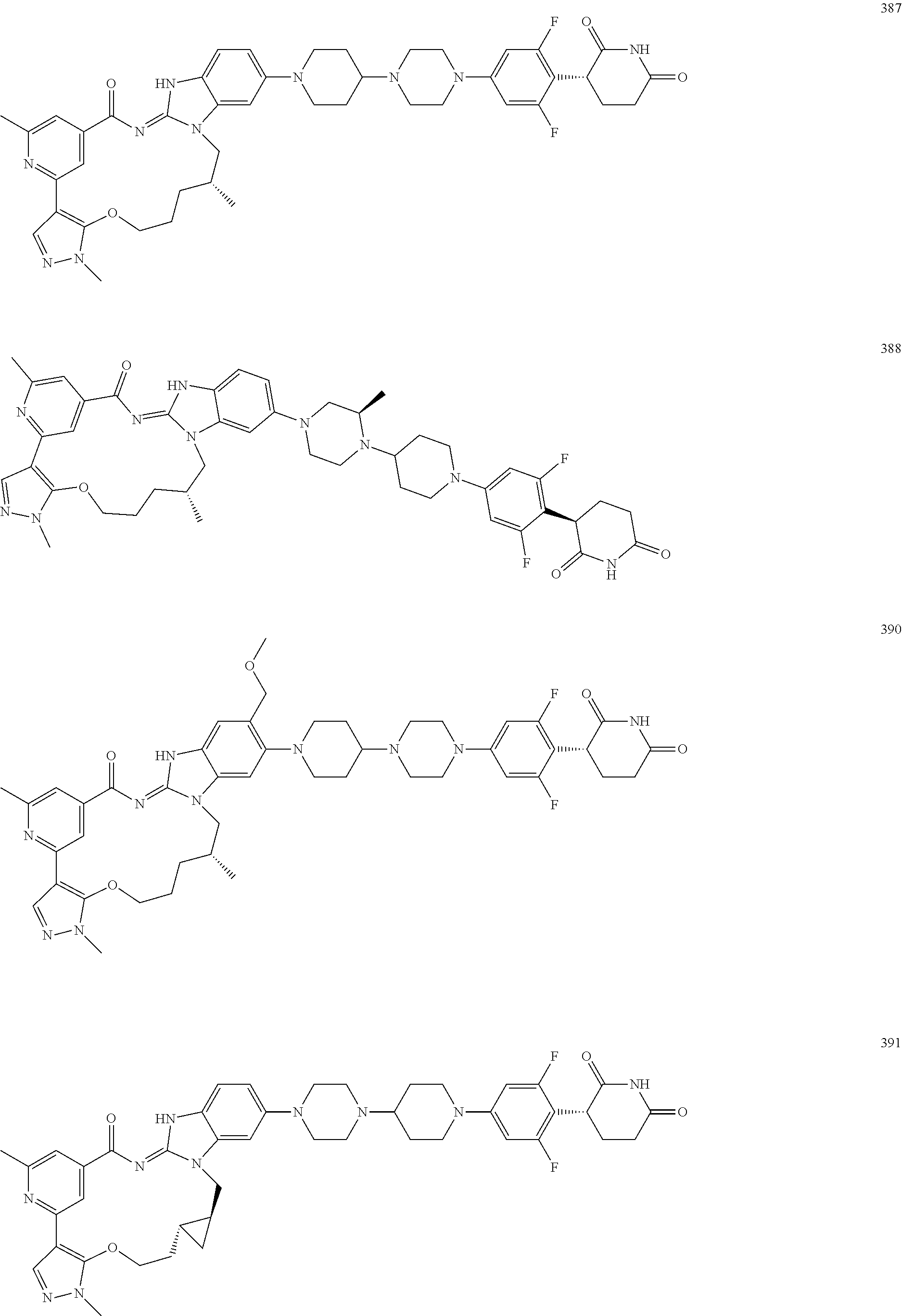 [SMILES: Cc1cc2cc(n1)-c1cnn(C)c1OCCC[C@@H](C)CN1/C(=N/C2=O)Nc2ccc(N3CCC(N4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@H](C)C3)cc21 ; COCc1cc2c(cc1N1CCC(N3CCN(c4cc(F)c([C@H]5CCC(=O)NC5=O)c(F)c4)CC3)CC1)N1C[C@H](C)CCCOc3c(cnn3C)-c3cc(cc(C)n3)C(=O)/N=C/1N2 ; Cc1cc2cc(n1)-c1cnn(C)c1OCC[C@H]1C[C@@H]1CN1/C(=N/C2=O)Nc2ccc(N3CCN(C4CCN(c5cc(F)c([C@H]6CCC(=O)NC6=O)c(F)c5)CC4)CC3)cc21]

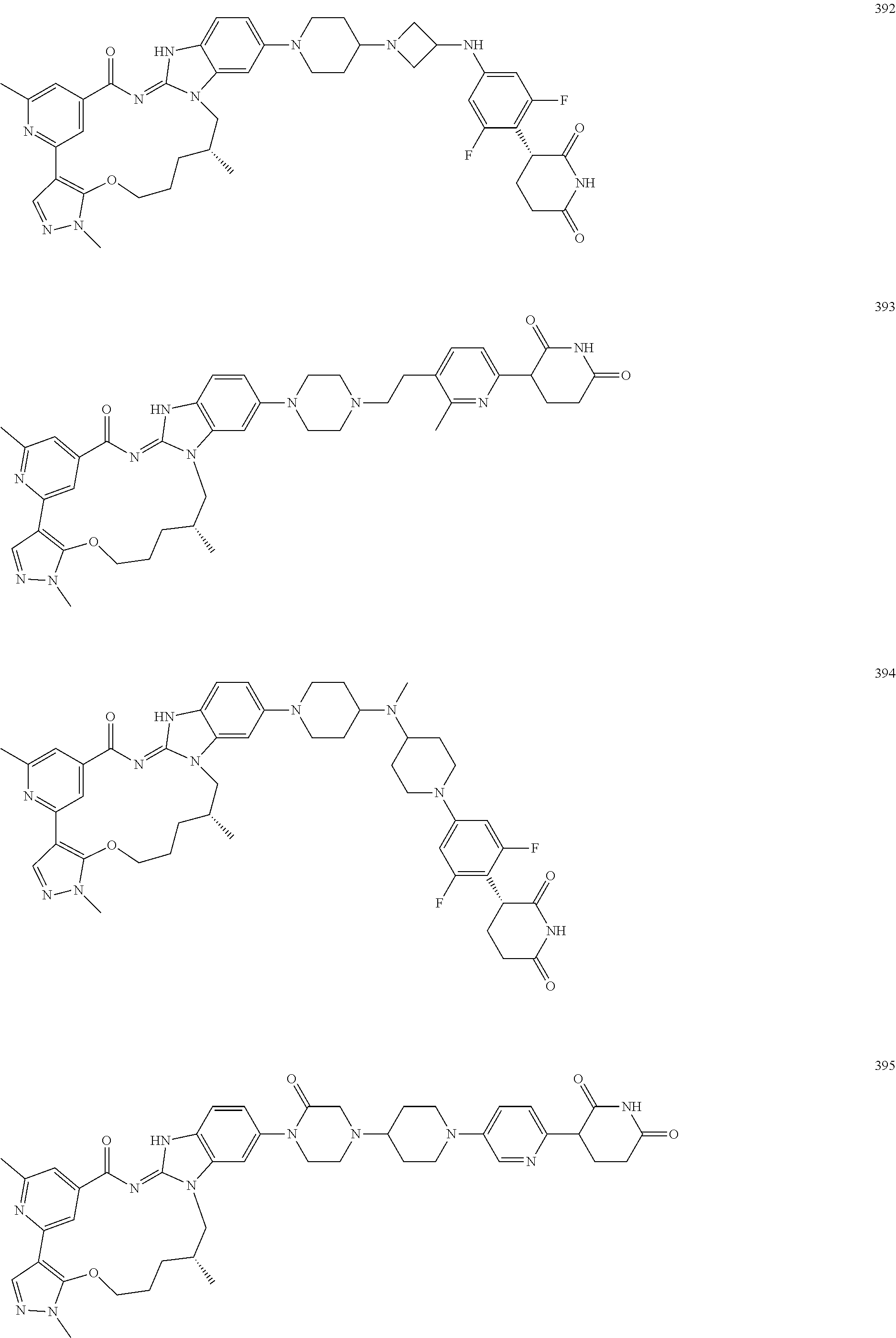 [SMILES: Cc1cc2cc(n1)-c1cnn(C)c1OCCC[C@@H](C)CN1/C(=N/C2=O)Nc2ccc(N3CCC(N4CC(Nc5cc(F)c([C@H]6CCC(=O)NC6=O)c(F)c5)C4)CC3)cc21 ; Cc1cc2cc(n1)-c1cnn(C)c1OCCC[C@@H](C)CN1/C(=N/C2=O)Nc2ccc(N3CCN(CCc4ccc(C5CCC(=O)NC5=O)nc4C)CC3)cc21 ; Cc1cc2cc(n1)-c1cnn(C)c1OCCC[C@@H](C)CN1/C(=N/C2=O)Nc2ccc(N3CCC(N(C)C4CCN(c5cc(F)c([C@H]6CCC(=O)NC6=O)c(F)c5)CC4)CC3)cc21 ; Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5)CC4)CC3=O)cc21]

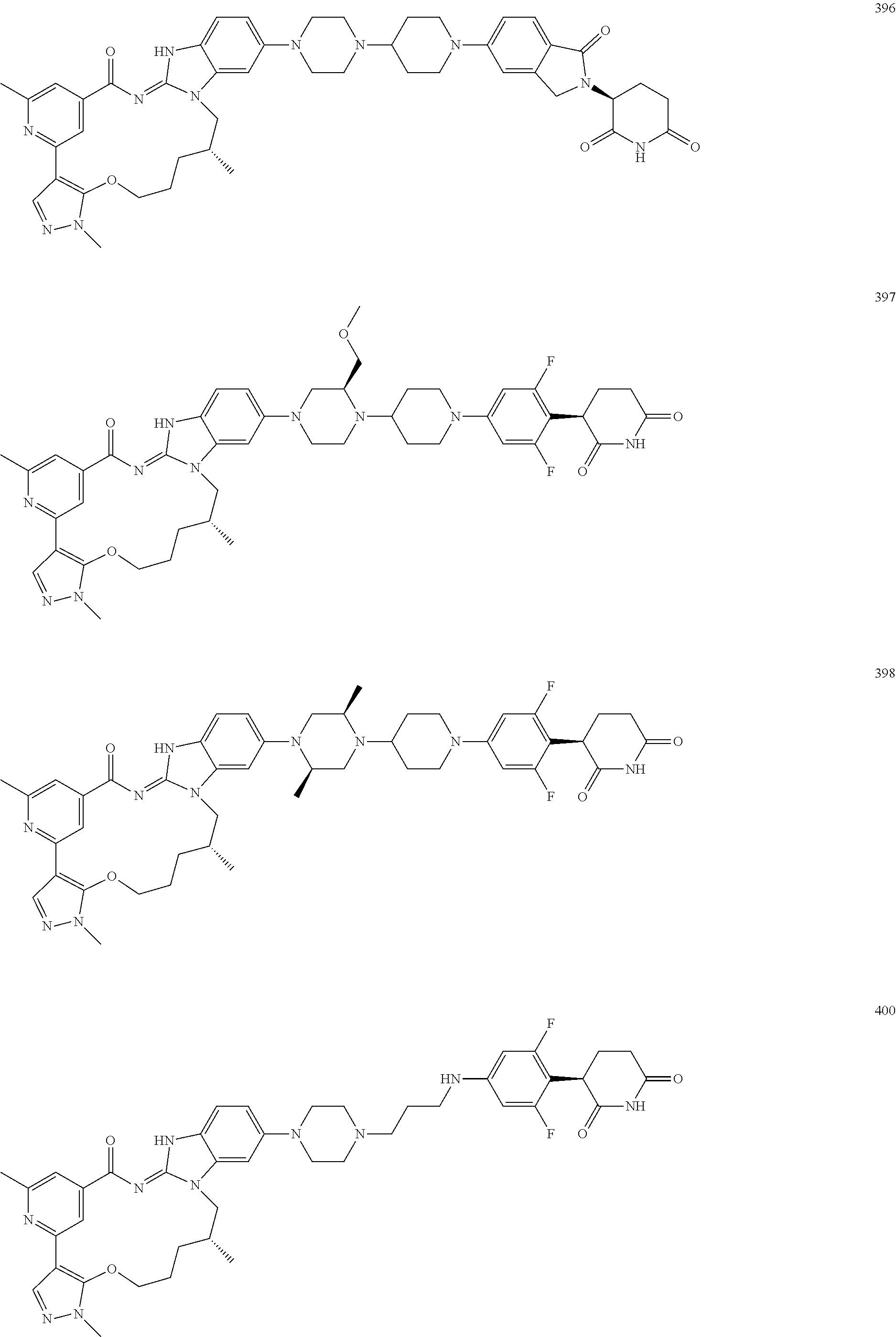 [SMILES: Cc1cc2cc(n1)-c1cnn(C)c1OCCC[C@@H](C)CN1/C(=N/C2=O)Nc2ccc(N3CCN(C4CCN(c5ccc6c(c5)CN([C@H]5CCC(=O)NC5=O)C6=O)CC4)CC3)cc21 ; COC[C@@H]1CN(c2ccc3c(c2)N2C[C@H](C)CCCOc4c(cnn4C)-c4cc(cc(C)n4)C(=O)/N=C/2N3)CCN1C1CCN(c2cc(F)c([C@H]3CCC(=O)NC3=O)c(F)c2)CC1 ; Cc1cc2cc(n1)-c1cnn(C)c1OCCC[C@@H](C)CN1/C(=N/C2=O)Nc2ccc(N3C[C@@H](C)N(C4CCN(c5cc(F)c([C@H]6CCC(=O)NC6=O)c(F)c5)CC4)C[C@H]3C)cc21 ; Cc1cc2cc(n1)-c1cnn(C)c1OCCC[C@@H](C)CN1/C(=N/C2=O)Nc2ccc(N3CCN(CCCNc4cc(F)c([C@H]5CCC(=O)NC5=O)c(F)c4)CC3)cc21]

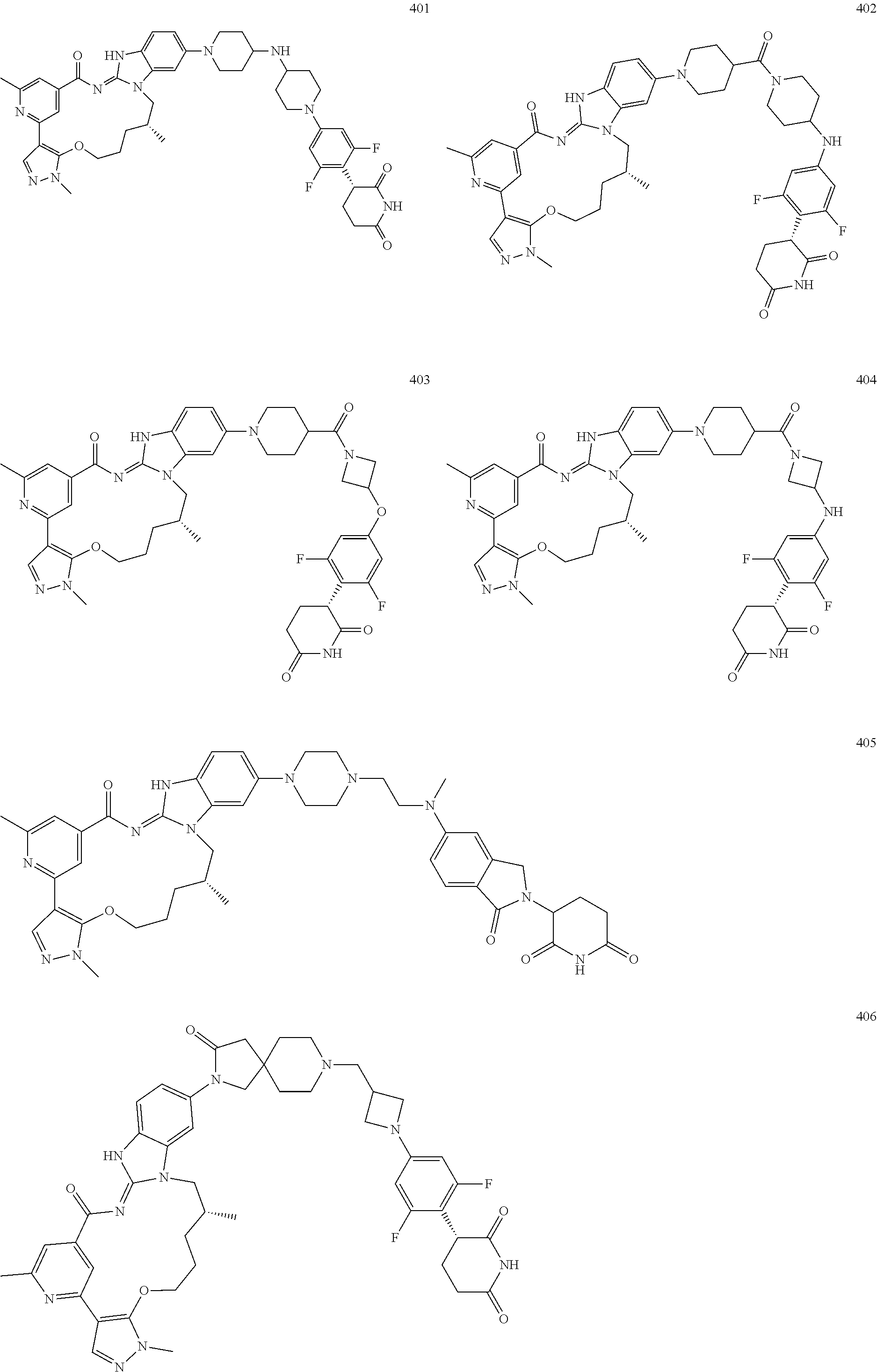

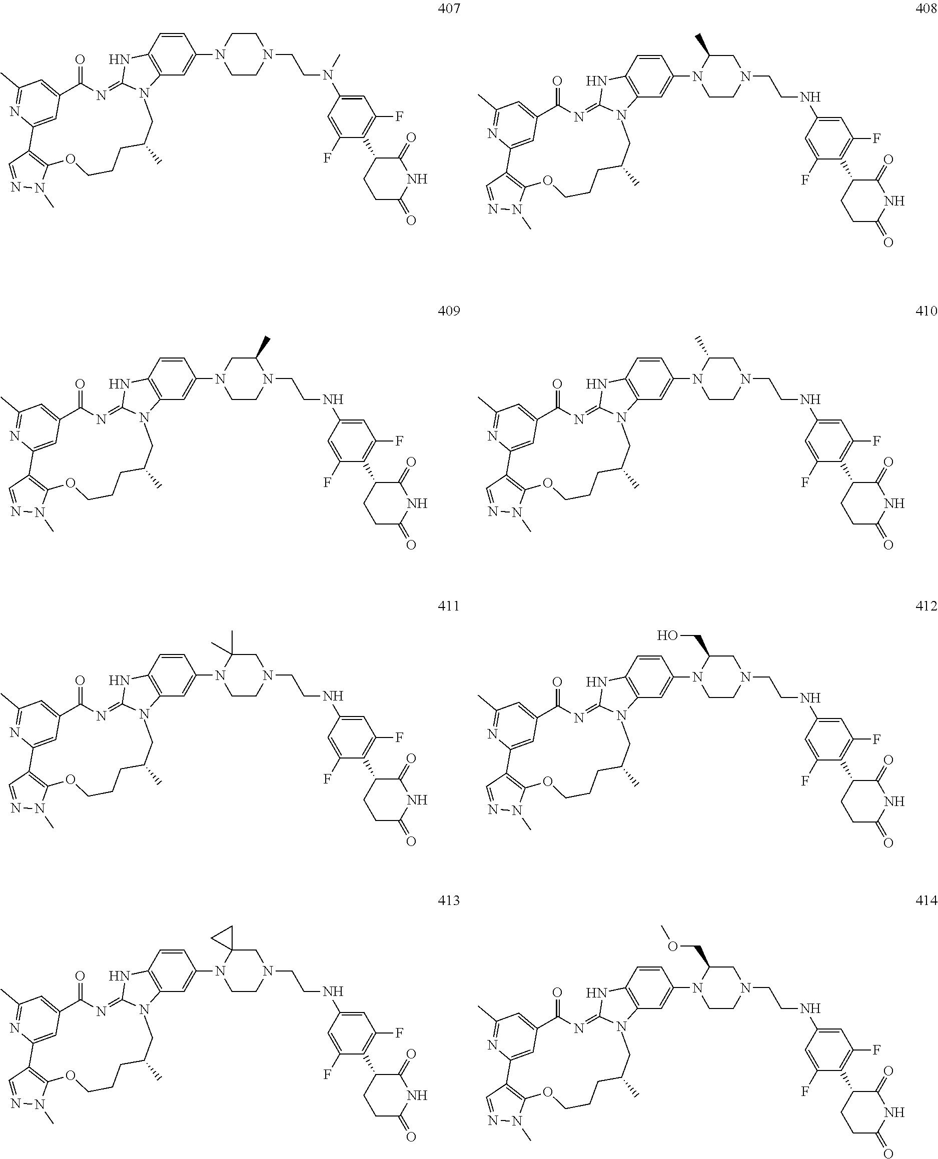

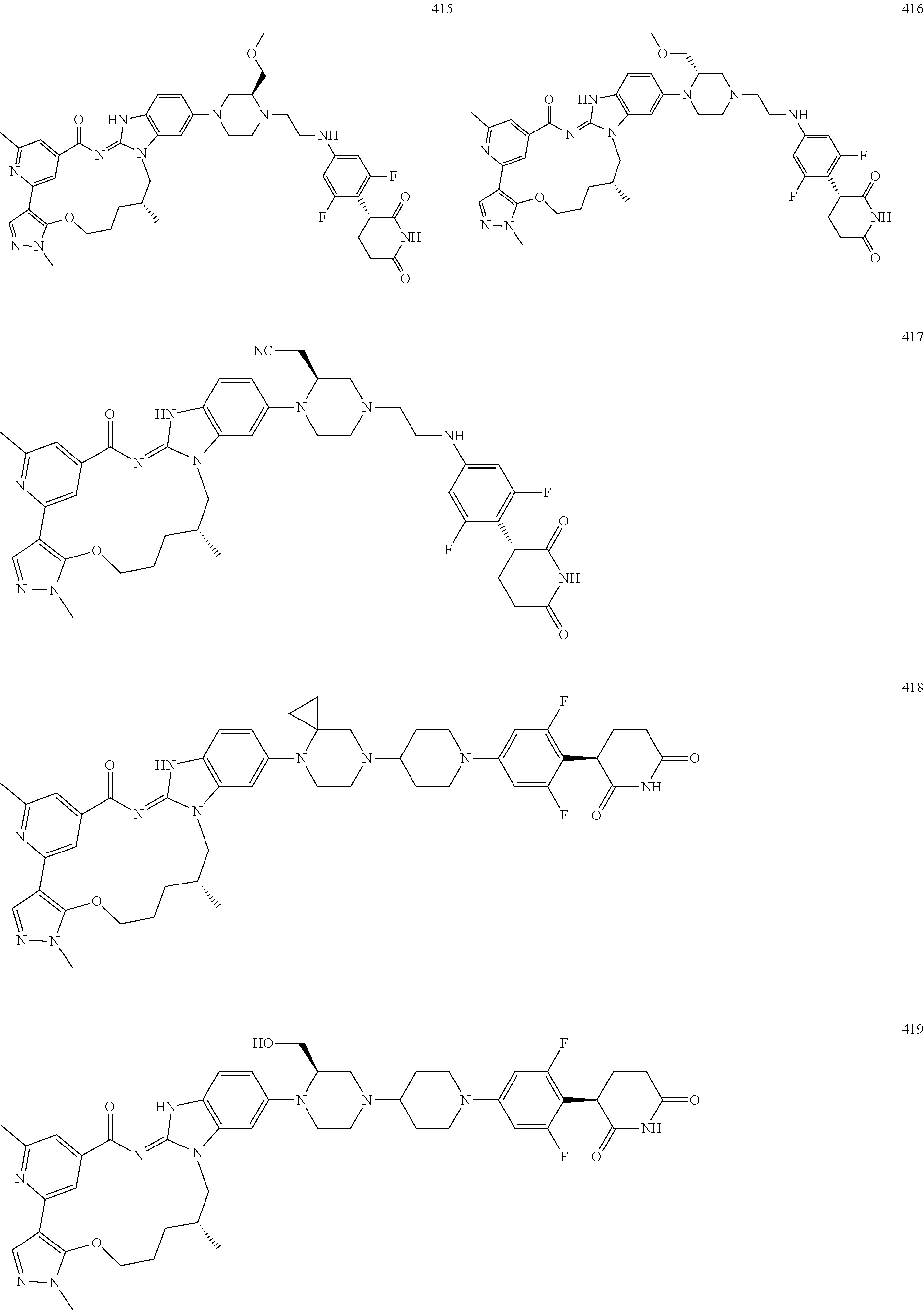

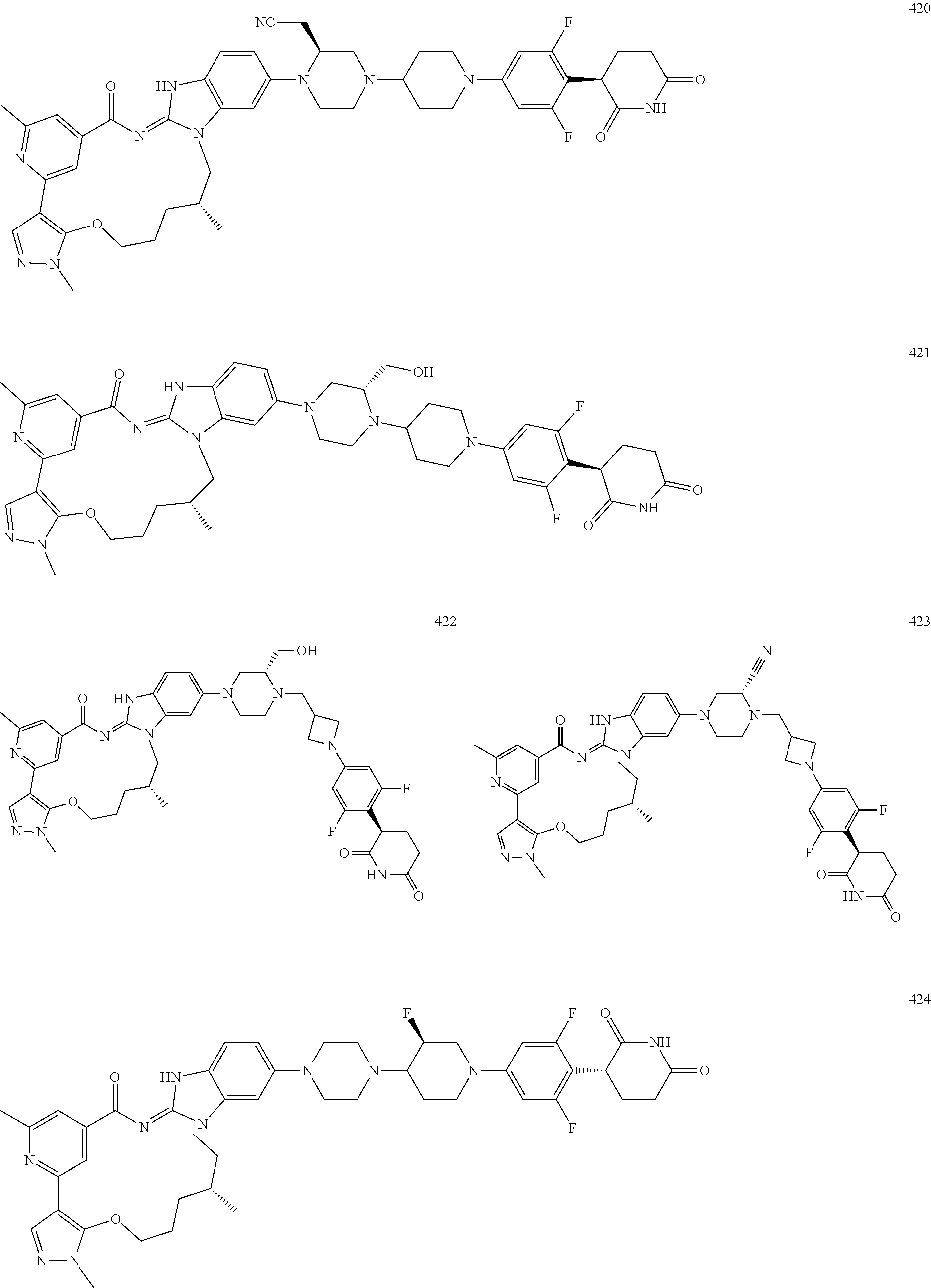

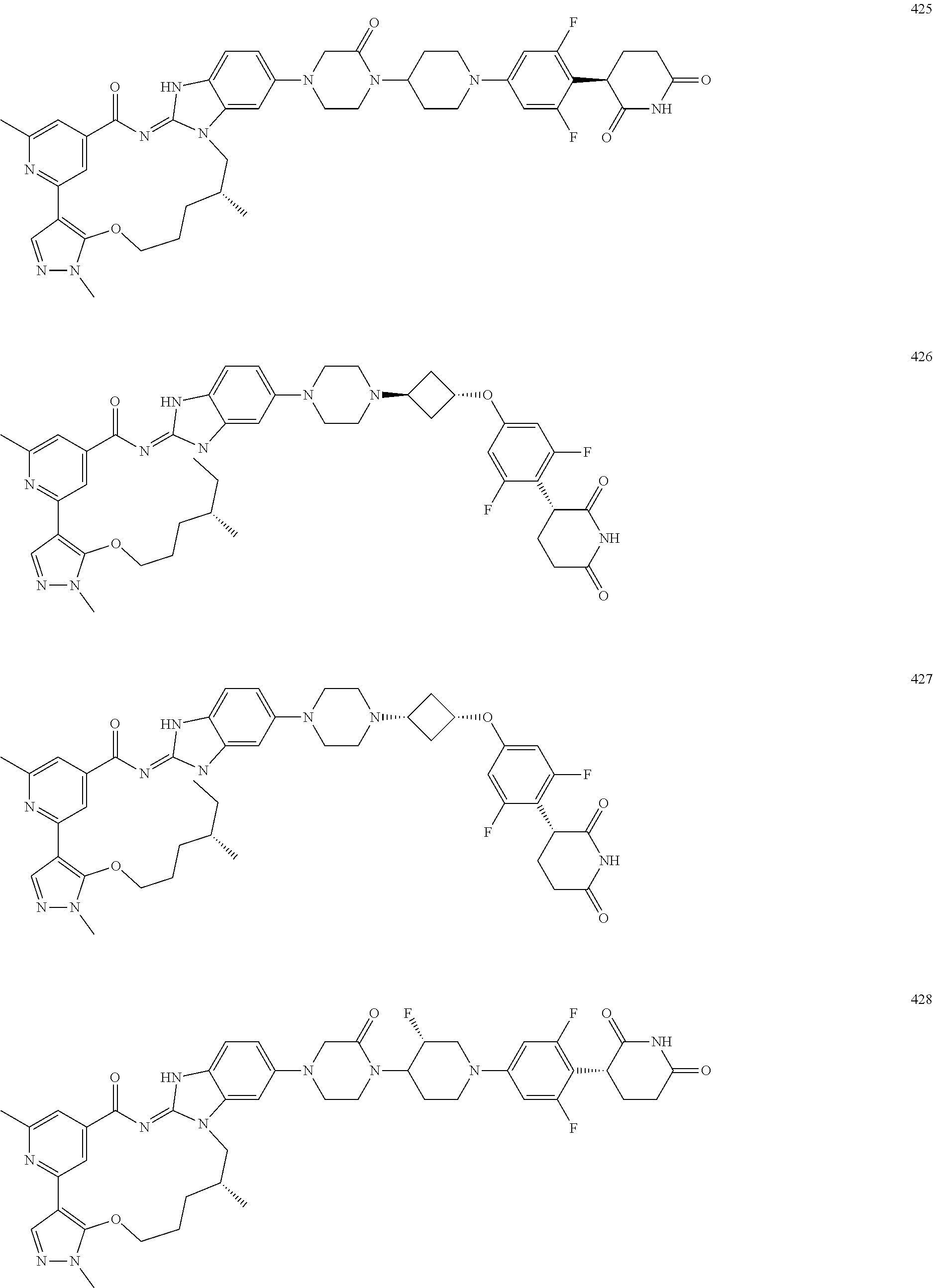

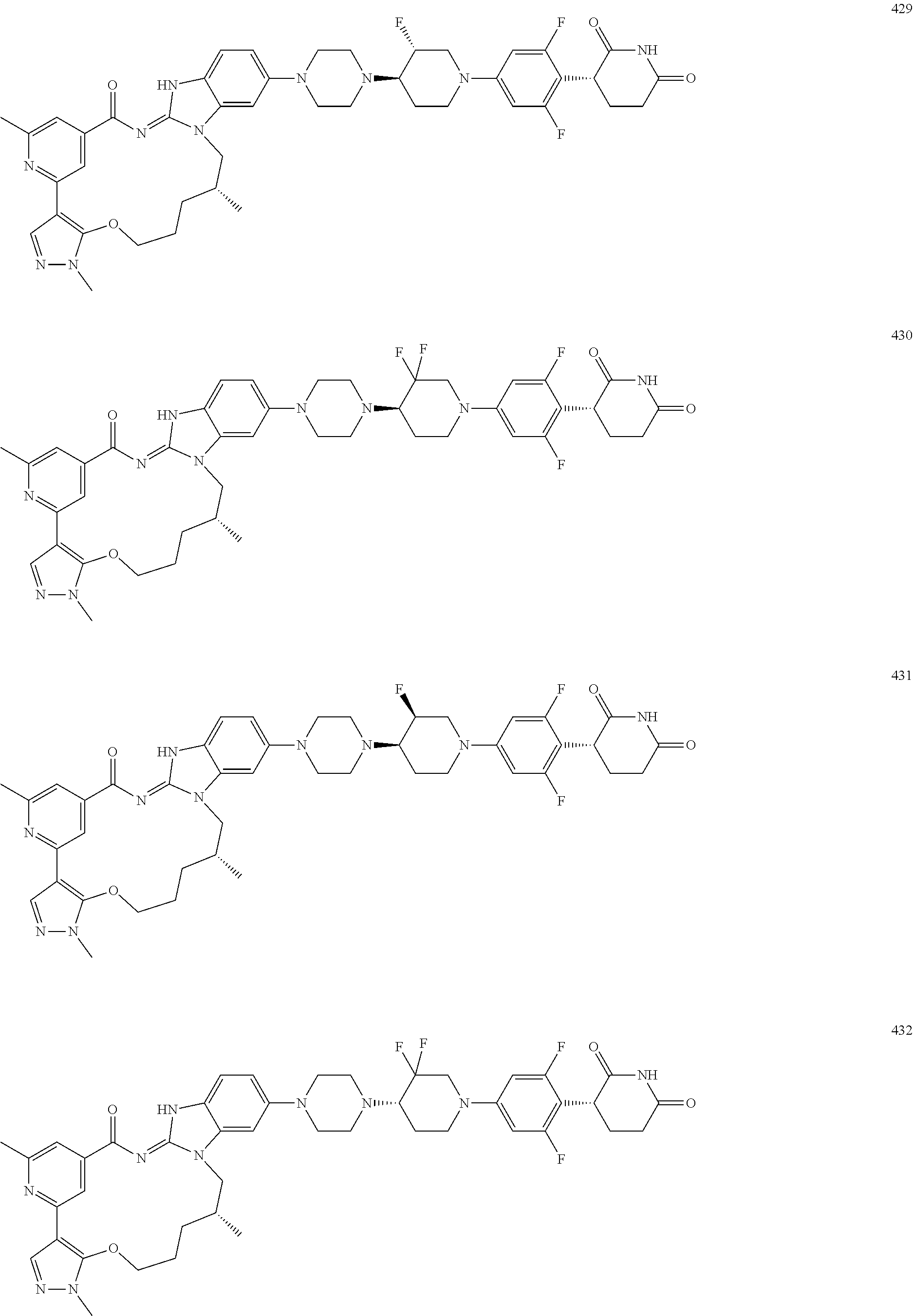

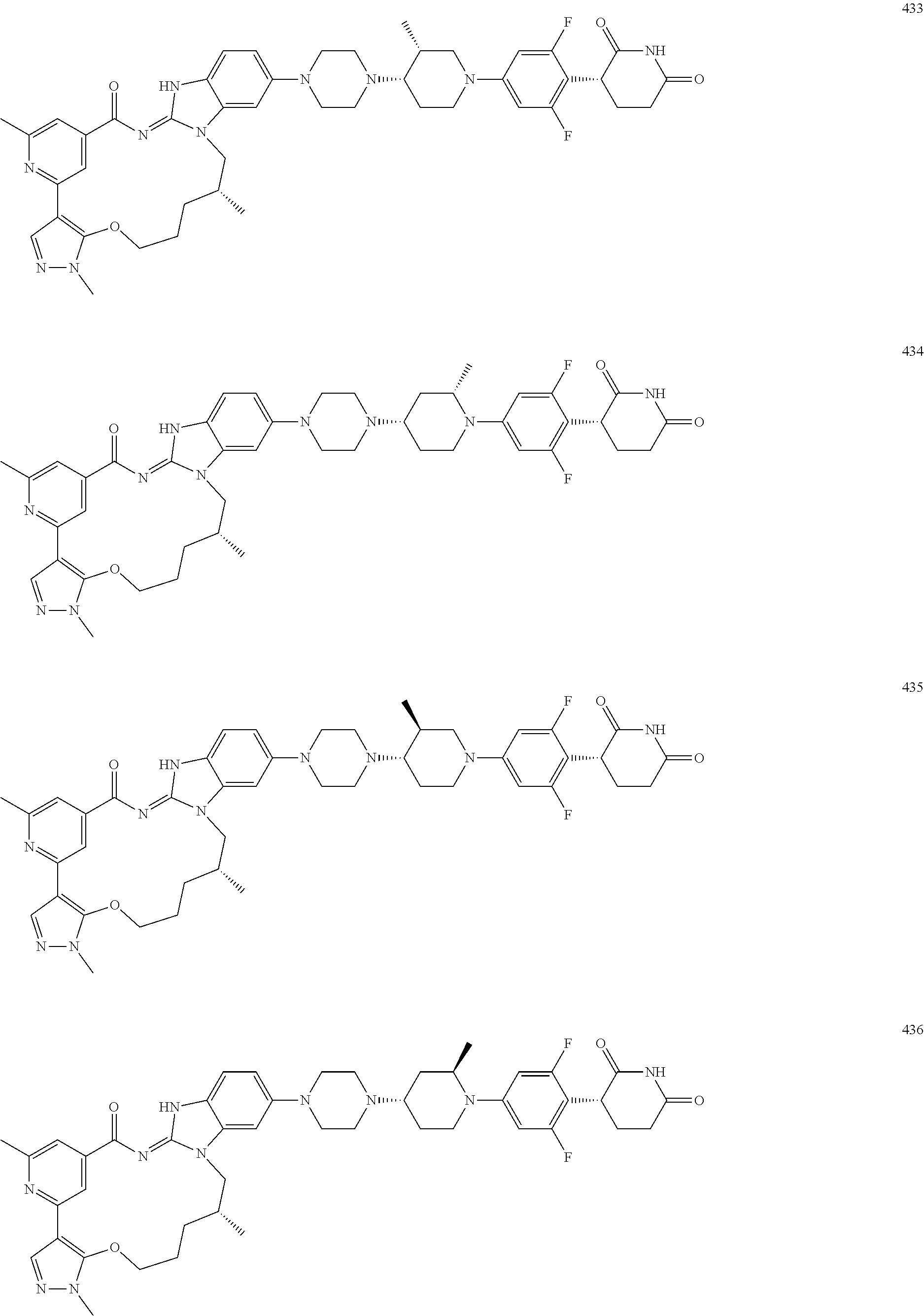

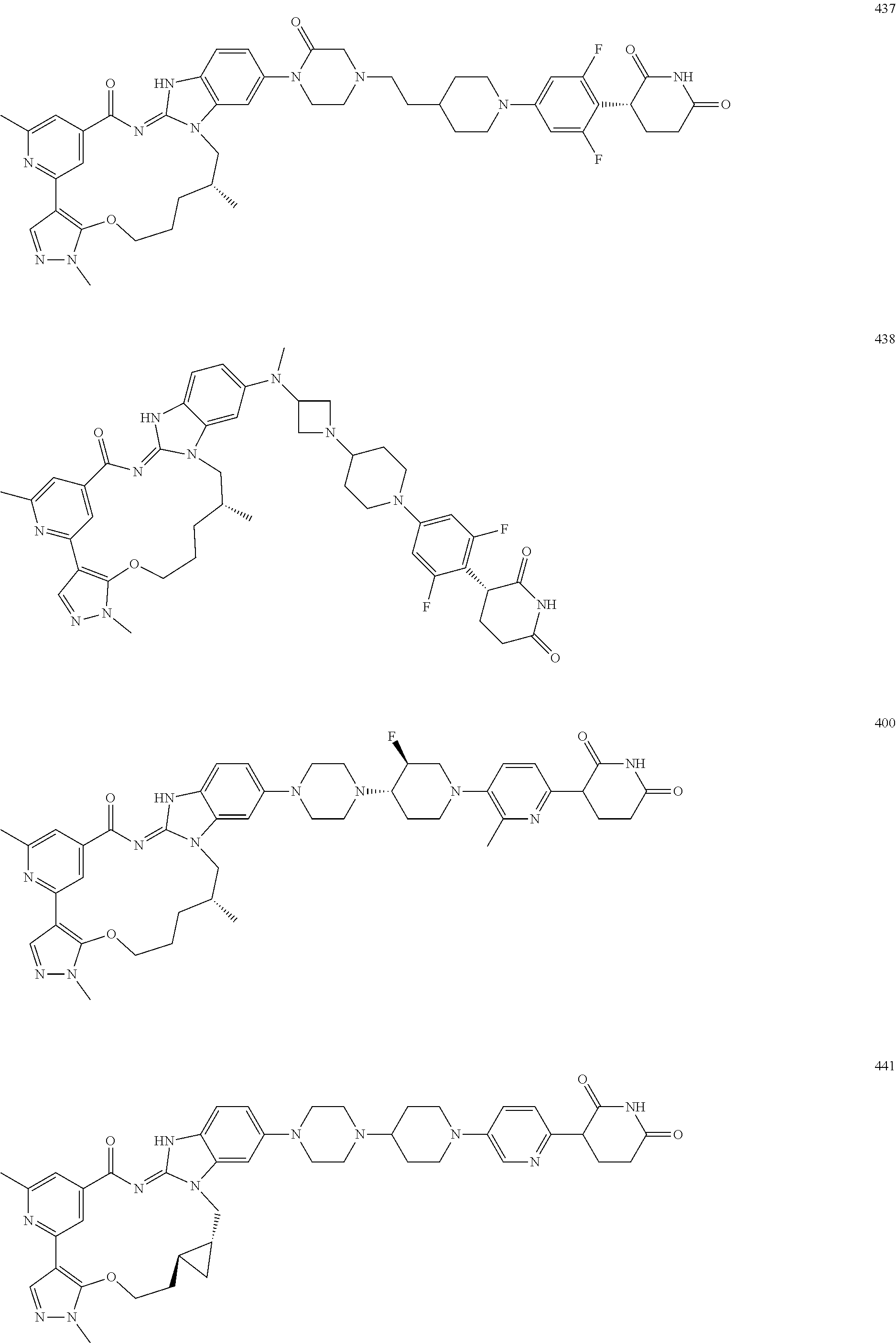 [SMILES: Cc1cc2cc(n1)-c1cnn(C)c1OCCC[C@@H](C)CN1/C(=N/C2=O)Nc2ccc(N3CCN(CCC4CCN(c5cc(F)c([C@H]6CCC(=O)NC6=O)c(F)c5)CC4)CC3=O)cc21 ; Cc1cc2cc(n1)-c1cnn(C)c1OCCC[C@@H](C)CN1/C(=N/C2=O)Nc2ccc(N(C)C3CN(C4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(N3CCN([C@H]4CCN(c5ccc(C6CCC(=O)NC6=O)nc5C)C[C@@H]4F)CC3)cc21 ; Cc1cc2cc(n1)-c1cnn(C)c1OCC[C@@H]1C[C@H]1CN1/C(=N/C2=O)Nc2ccc(N3CCN(C4CCN(c5ccc(C6CCC(=O)NC6=O)nc5)CC4)CC3)cc21]

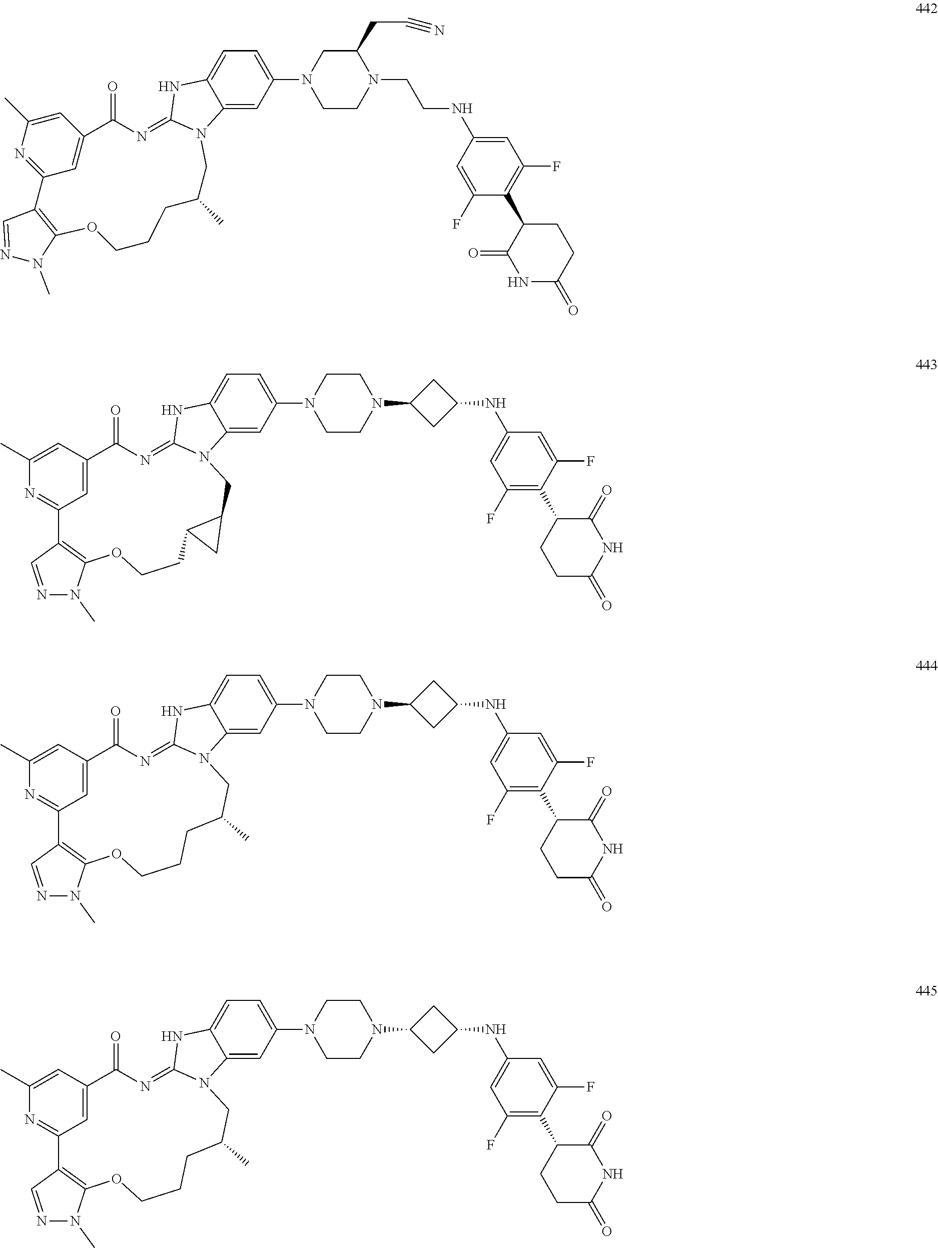

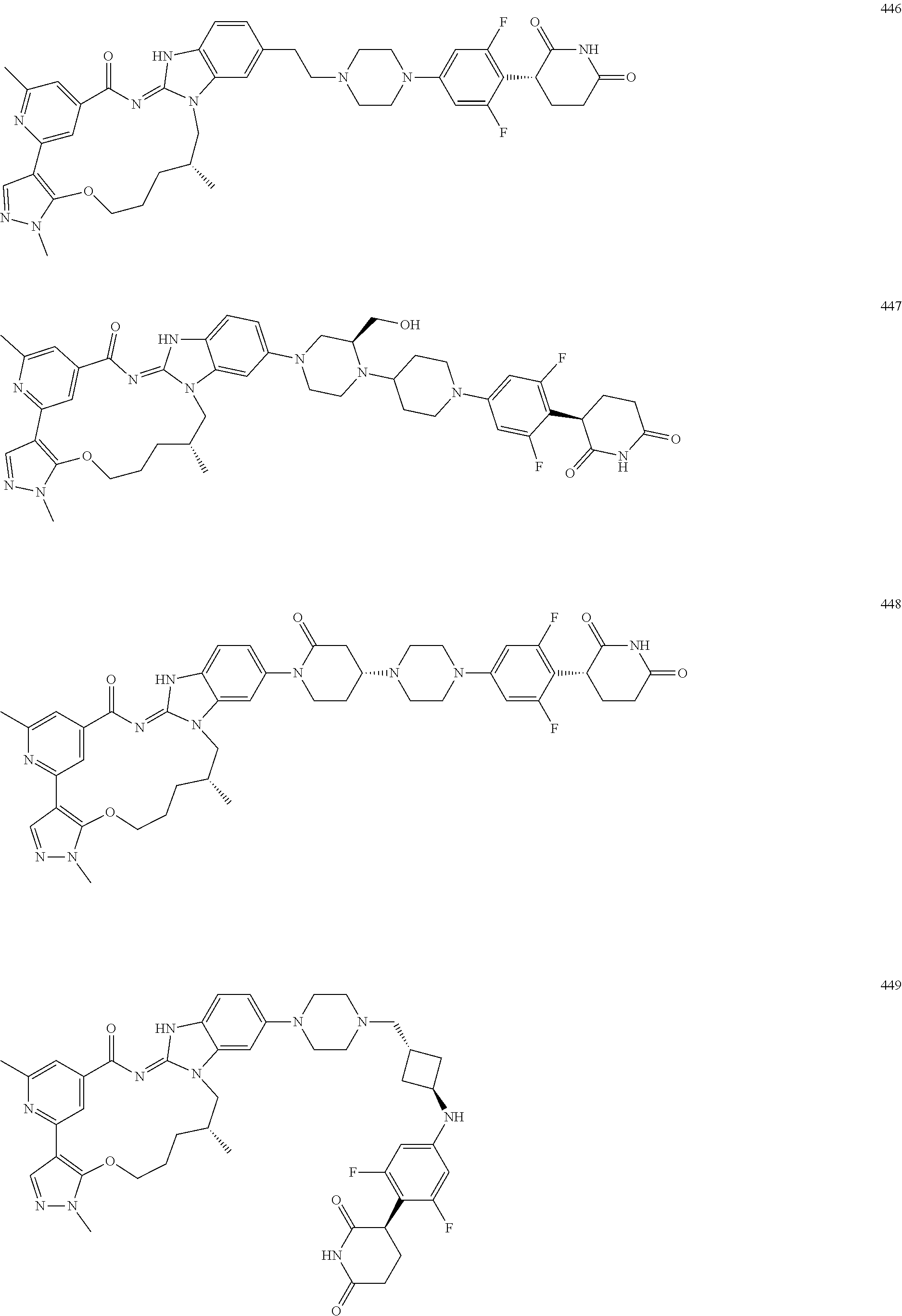 [SMILES: Cc1cc2cc(n1)-c1cnn(C)c1OCCC[C@@H](C)CN1/C(=N/C2=O)Nc2ccc(CCN3CCN(c4cc(F)c([C@H]5CCC(=O)NC5=O)c(F)c4)CC3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@H](CO)C3)cc21 ; Cc1cc2cc(n1)-c1cnn(C)c1OCCC[C@@H](C)CN1/C(=N/C2=O)Nc2ccc(N3CC[C@@H](N4CCN(c5cc(F)c([C@H]6CCC(=O)NC6=O)c(F)c5)CC4)CC3=O)cc21 ; Cc1cc2cc(n1)-c1cnn(C)c1OCCC[C@@H](C)CN1/C(=N/C2=O)Nc2ccc(N3CCN(C[C@H]4C[C@H](Nc5cc(F)c([C@H]6CCC(=O)NC6=O)c(F)c5)C4)CC3)cc21]

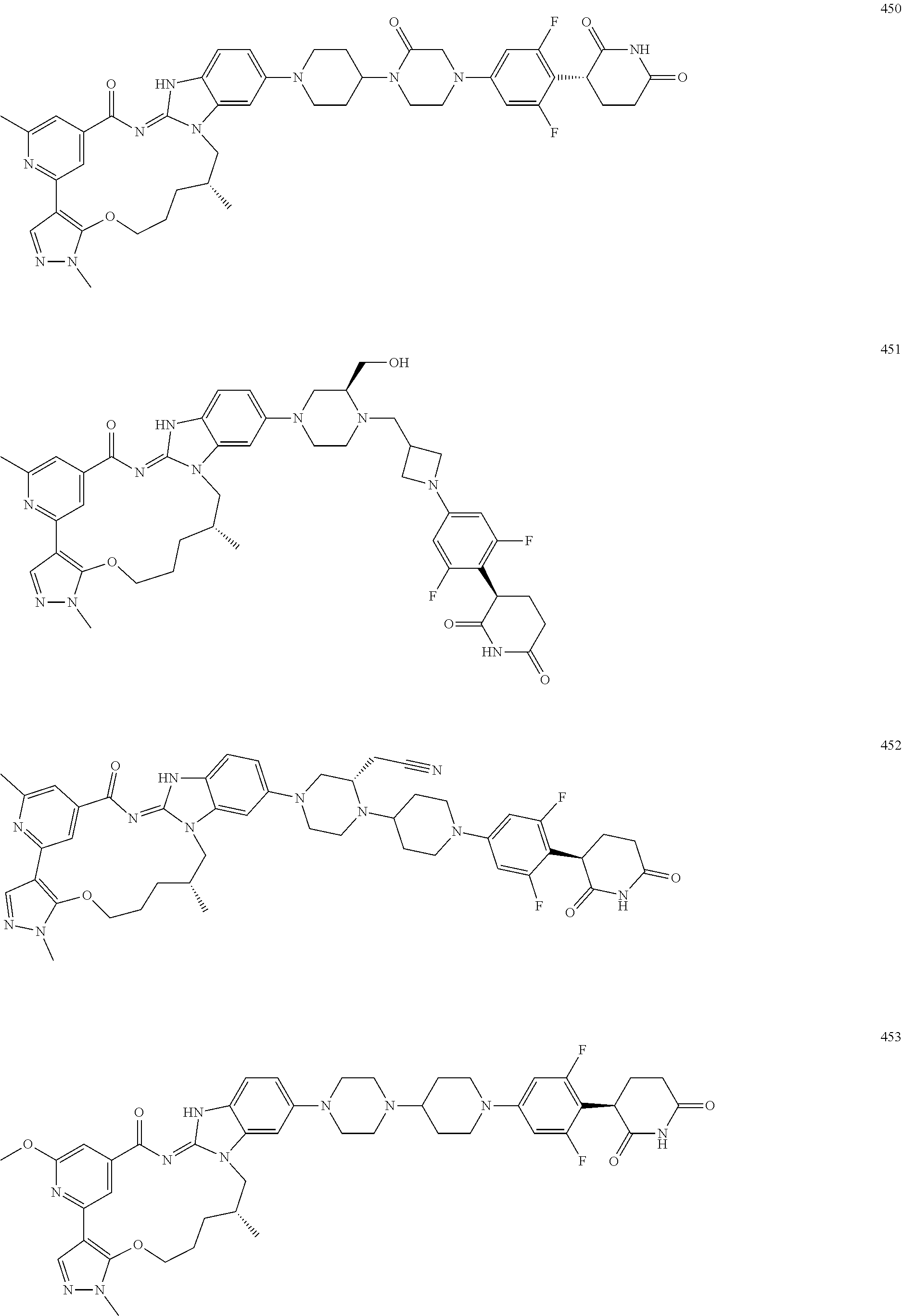 [SMILES: Cc1cc2cc(n1)-c1cnn(C)c1OCCC[C@@H](C)CN1/C(=N/C2=O)Nc2ccc(N3CCC(N4CCN(c5cc(F)c([C@H]6CCC(=O)NC6=O)c(F)c5)CC4=O)CC3)cc21 ; Cc1cc2cc(n1)-c1cnn(C)c1OCCC[C@@H](C)CN1/C(=N/C2=O)Nc2ccc(N3CCN(CC4CN(c5cc(F)c([C@H]6CCC(=O)NC6=O)c(F)c5)C4)[C@H](CO)C3)cc21 ; Cc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)[C@@H](CC#N)C3)cc21 ; COc1cc2cc(n1)-c1cnn(C)c1OCCC[C@@H](C)CN1/C(=N/C2=O)Nc2ccc(N3CCN(C4CCN(c5cc(F)c([C@H]6CCC(=O)NC6=O)c(F)c5)CC4)CC3)cc21]

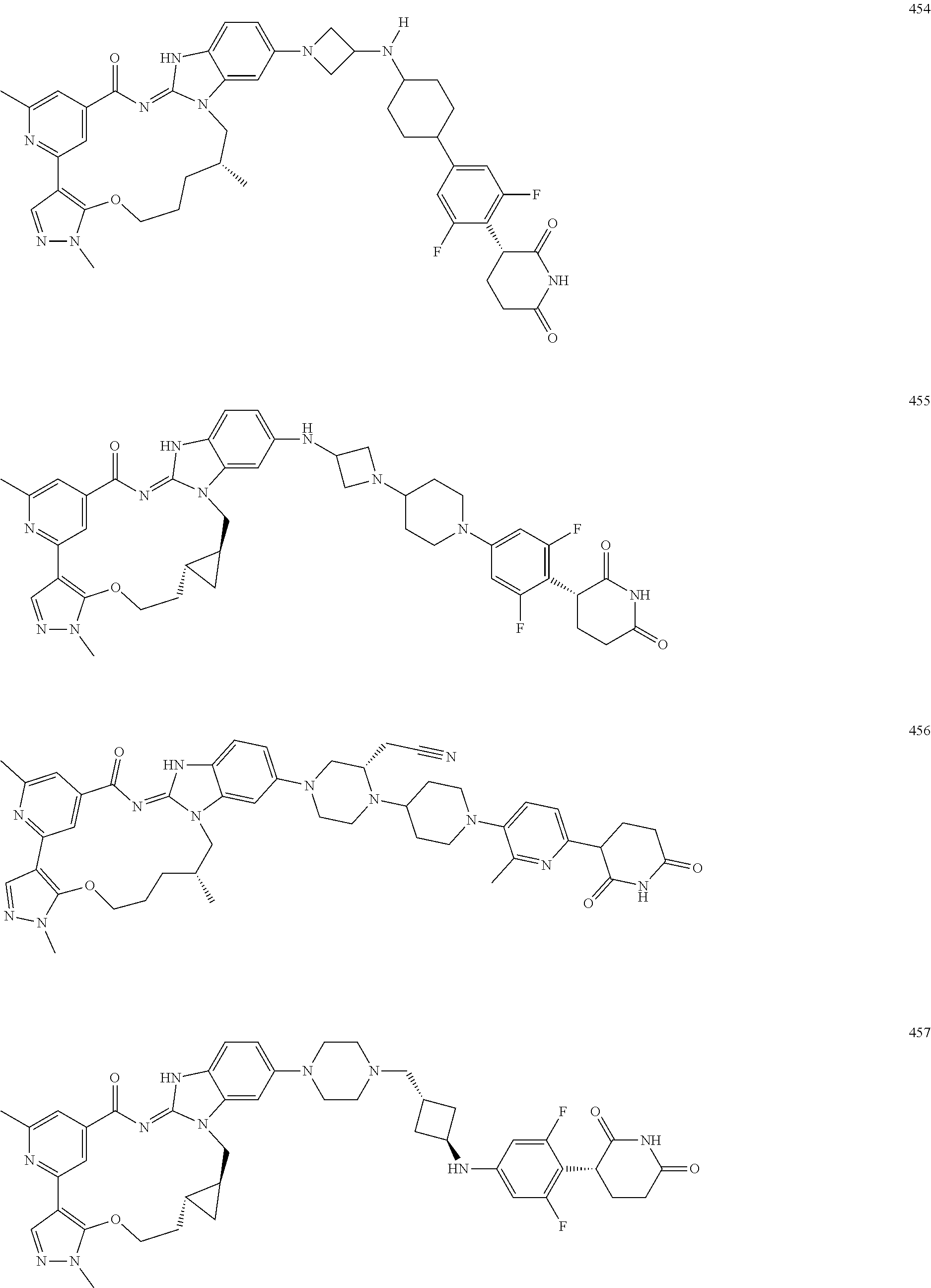 [SMILES: Cc1cc2cc(n1)-c1cnn(C)c1OCCC[C@@H](C)CN1/C(=N/C2=O)Nc2ccc(N3CC(NC4CCC(c5cc(F)c([C@H]6CCC(=O)NC6=O)c(F)c5)CC4)C3)cc21 ; Cc1cc2cc(n1)-c1cnn(C)c1OCC[C@H]1C[C@@H]1CN1/C(=N/C2=O)Nc2ccc(NC3CN(C4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(N3CCN(C4CCN(c5ccc(C6CCC(=O)NC6=O)nc5C)CC4)[C@@H](CC#N)C3)cc21 ; Cc1cc2cc(n1)-c1cnn(C)c1OCC[C@H]1C[C@@H]1CN1/C(=N/C2=O)Nc2ccc(N3CCN(C[C@H]4C[C@H](Nc5cc(F)c([C@H]6CCC(=O)NC6=O)c(F)c5)C4)CC3)cc21]

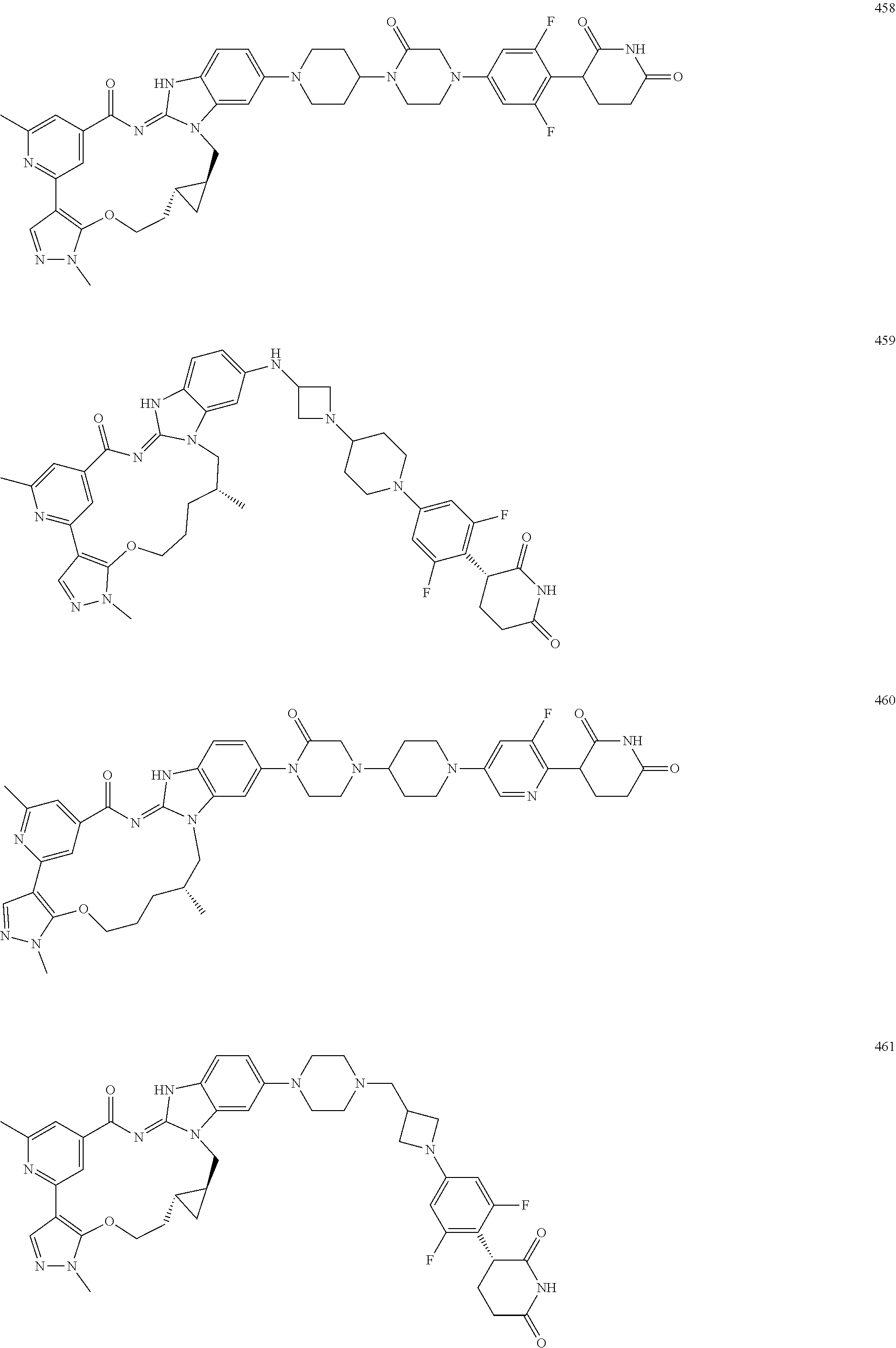 [SMILES: Cc1cc2cc(n1)-c1cnn(C)c1OCC[C@H]1C[C@@H]1CN1/C(=N/C2=O)Nc2ccc(N3CCC(N4CCN(c5cc(F)c(C6CCC(=O)NC6=O)c(F)c5)CC4=O)CC3)cc21 ; Cc1cc2cc(n1)-c1cnn(C)c1OCCC[C@@H](C)CN1/C(=N/C2=O)Nc2ccc(NC3CN(C4CCN(c5cc(F)c([C@H]6CCC(=O)NC6=O)c(F)c5)CC4)C3)cc21 ; Cc1cc2cc(n1)-c1cnn(C)c1OCCC[C@@H](C)CN1/C(=N/C2=O)Nc2ccc(N3CCN(C4CCN(c5cnc(C6CCC(=O)NC6=O)c(F)c5)CC4)CC3=O)cc21 ; Cc1cc2cc(n1)-c1cnn(C)c1OCC[C@H]1C[C@@H]1CN1/C(=N/C2=O)Nc2ccc(N3CCN(CC4CN(c5cc(F)c([C@H]6CCC(=O)NC6=O)c(F)c5)C4)CC3)cc21]

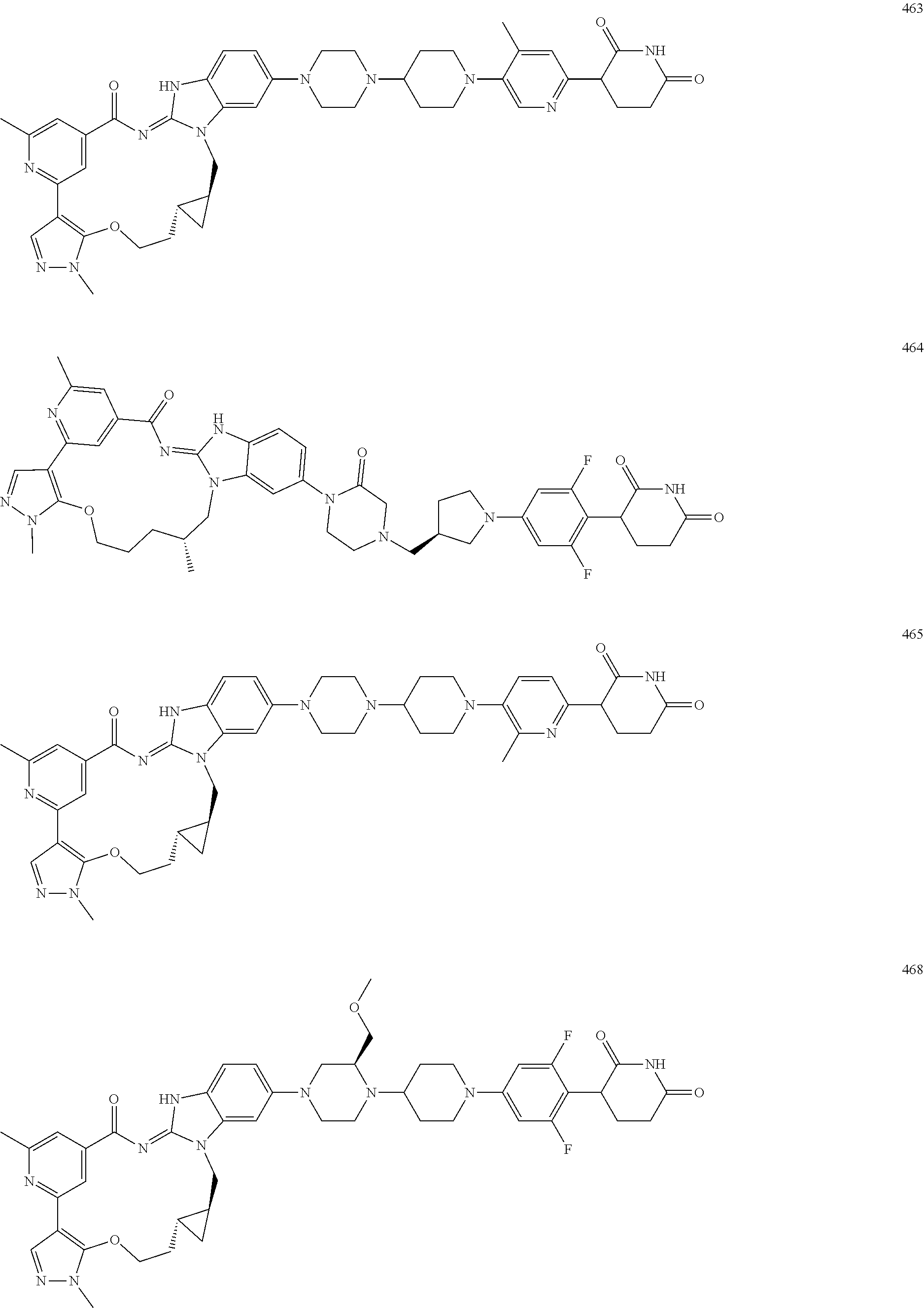 [SMILES: Cc1cc2cc(n1)-c1cnn(C)c1OCC[C@H]1C[C@@H]1CN1/C(=N/C2=O)Nc2ccc(N3CCN(C4CCN(c5cnc(C6CCC(=O)NC6=O)cc5C)CC4)CC3)cc21 ; Cc1cc2cc(n1)-c1cnn(C)c1OCCC[C@@H](C)CN1/C(=N/C2=O)Nc2ccc(N3CCN(C[C@H]4CCN(c5cc(F)c(C6CCC(=O)NC6=O)c(F)c5)C4)CC3=O)cc21 ; Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)CC3)cc21 ; COC[C@@H]1CN(c2ccc3c(c2)N2C[C@H]4C[C@@H]4CCOc4c(cnn4C)-c4cc(cc(C)n4)C(=O)/N=C/2N3)CCN1C1CCN(c2cc(F)c(C3CCC(=O)NC3=O)c(F)c2)CC1]

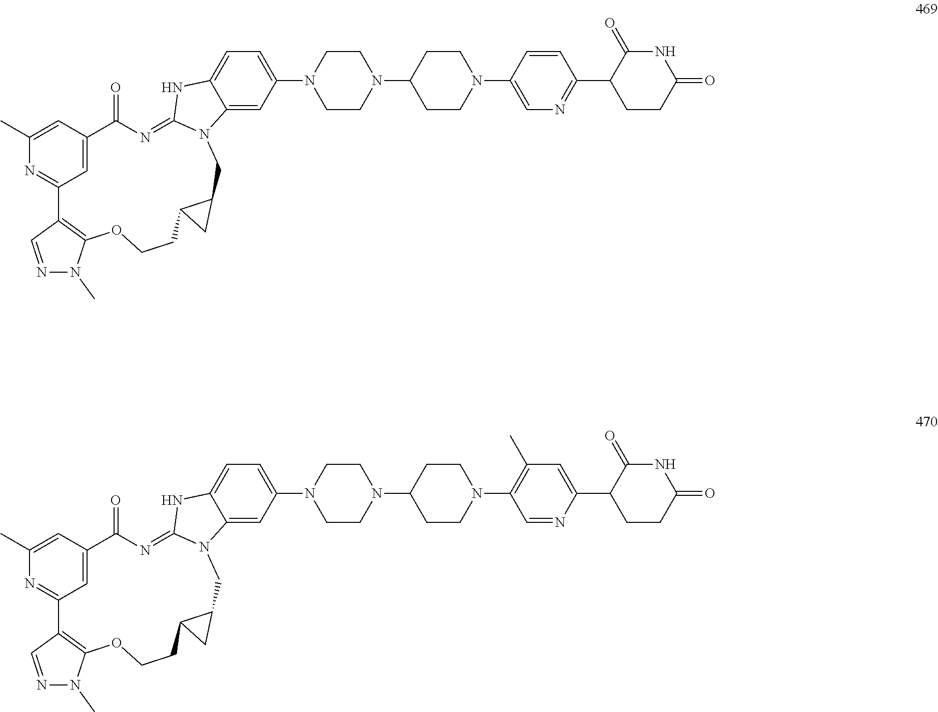 [SMILES: Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5)CC4)CC3)cc21 ; Cc1cc2cc(n1)-c1cnn(C)c1OCC[C@@H]1C[C@H]1CN1/C(=N/C2=O)Nc2ccc(N3CCN(C4CCN(c5cnc(C6CCC(=O)NC6=O)cc5C)CC4)CC3)cc21]

53. A pharmaceutical composition comprising a compound of any one of claims 1-52 or a pharmaceutically acceptable salt, stereoisomer, tautomer or prodrug thereof, together with a pharmaceutically acceptable excipient.

54. A method of treating a disease that can be affected by EGFR modulation, comprises administrating a subject in need thereof an effective amount of a compound of any one of claims 1-52 or a pharmaceutically acceptable salt, stereoisomer, tautomer or prodrug thereof.

55. The method of claim 52, wherein the disease is selected from cancer, preferred pancreatic cancer, breast cancer, glioblastoma multiforme, head and neck cancer, or non-small cell lung cancer.

56. Use of a compound of any one of claims 1-52 or a pharmaceutically acceptable salt, stereoisomer, tautomer or prodrug thereof in the preparation of a medicament for treating a disease that can be affected by EGFR modulation.

57. The use of claim 56, wherein the disease is cancer, preferred pancreatic cancer, breast cancer, glioblastoma multiforme, head and neck cancer, or non-small cell lung cancer.
